(19) United States
(12) Patent Application Publication
(10) Pub. No.: US 2026/0103465 A1
(43) Pub. Date: Apr. 16, 2026

(54) GLYCOGEN SYNTHASE KINASE 3 INHIBITORS AND USES THEREOF

(71) Applicants: The Broad Institute, Inc., Cambridge, MA (US); Biogen MA Inc., Cambridge, MA (US)

(72) Inventors: Florence Fevrier Wagner, Cambridge, MA (US); Kwaku Kyei-Baffour, Cambridge, MA (US); Michel Weiwer, Cambridge, MA (US); TeYu Chen, Cambridge, MA (US); Zain Yousaf, Cambridge, MA (US)

(73) Assignees: The Broad Institute, Inc., Cambridge, MA (US); Biogen MA Inc., Cambridge, MA (US)

(21) Appl. No.: 19/418,475

(22) Filed: Dec. 12, 2025

Related U.S. Application Data
(63) Continuation of application No. PCT/US2024/033922, filed on Jun. 13, 2024.
(60) Provisional application No. 63/508,264, filed on Jun. 14, 2023.

(51) Int. Cl. C07D 471/04; A61K 31/4375; A61K 31/438; A61K 31/444; A61K 31/4709; A61K 31/501; A61K 31/506; A61K 31/5377; C07D 471/10; C07D 471/14; C07D 471/20; C07D 491/20

(52) U.S. Cl. CPC C07D 471/04; A61K 31/4375; A61K 31/438; A61K 31/444; A61K 31/4709; A61K 31/501; A61K 31/506; A61K 31/5377; C07D 471/10; C07D 471/14; C07D 471/20; C07D 491/20

(57) ABSTRACT

The present disclosure provides compounds of Formula I, and pharmaceutically acceptable salts, solvates, hydrates, polymorphs, co-crystals, tautomers, stereoisomers, isotopically labeled compounds, and prodrugs thereof. The provided compounds may be glycogen synthase kinase 3 (GSK3) inhibitors. The present disclosure also provides pharmaceutical compositions, combination therapies, and kits comprising the compounds, and pharmaceutically acceptable salts, solvates, hydrates, polymorphs, co-crystals, tautomers, stereoisomers, isotopically labeled compounds, or prodrugs thereof, and methods of treating or preventing diseases and disorders associated with GSK3.

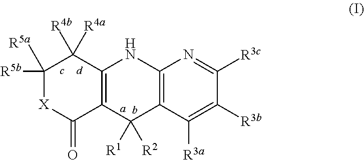

### RELATED APPLICATIONS

The present application claims priority under 35 U.S.C. § 119(e) to U.S. Provisional Patent Application No. 63/508,264, filed Jun. 14, 2023, which is incorporated herein by reference.

### BACKGROUND

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-I), tumor necrosis factor α (TNF-α)), and growth factors (e.g., granulocyte macrophage-colony-stimulating factor (GM-CSF), fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, metabolic disorders (e.g., diabetes), and hormone-related diseases. Accordingly, there remains a need to find protein kinase inhibitors, particularly glycogen synthase kinase 3 (GSK3) inhibitors, useful as therapeutic agents. GSK3 inhibitors have been reported in, e.g., U.S. patent application publication numbers US-2014-0107141-A1, US-2016-0375006-A1, and US-2020-0109154-A1, each of which is incorporated herein by reference in its entirety.

### SUMMARY OF THE DISCLOSURE

The present disclosure relates in part to compounds (e.g., compounds of Formula I, and pharmaceutically acceptable salts, solvates, hydrates, polymorphs, co-crystals, tautomers, stereoisomers, isotopically labeled compounds, and prodrugs thereof). The compounds provided herein may inhibit GSK3. The compounds provided herein may selectively inhibit GSK3 (e.g., selectively inhibit GSK3α (GSK3a) over GSK3β (GSK3b) or selectively inhibit GSK3β over GSK3α). The compounds provided herein may be advantageous over known GSK3 inhibitors (e.g., non-selective GSK3 inhibitors) at least in part because the former may reduce or eliminate off-target effects. The compounds provided herein may also be advantageous over known GSK3 inhibitors in that the former may be more permeable than the latter (e.g., the former may pass through the blood-brain barrier (BBB) more easily than the latter). The present disclosure also provides pharmaceutical compositions and kits comprising the compounds provided herein. The present disclosure also provides methods of treating or preventing a disease, as well as methods of inhibiting the activity and/or production of a GSK3.

In one aspect, the present disclosure provides compounds of Formula I:

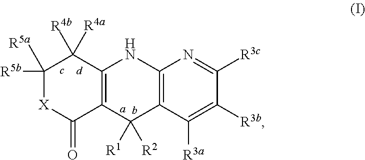

and pharmaceutically acceptable salts, solvates, hydrates, polymorphs, co-crystals, tautomers, stereoisomers, isotopically labeled compounds, and prodrugs thereof.

In another aspect, the present disclosure provides pharmaceutical compositions comprising a compound provided herein and optionally a pharmaceutically acceptable excipient.

In another aspect, the present disclosure provides kits comprising a compound provided herein or pharmaceutical composition provided herein and instructions for using the compound or pharmaceutical composition.

In another aspect, the present disclosure provides methods for treating diseases in a subject in need thereof, the methods comprising administering to the subject an effective amount of a compound or pharmaceutical composition provided herein.

In another aspect, the present disclosure provides methods for preventing diseases in a subject in need thereof, the methods comprising administering to the subject an effective amount of a compound or pharmaceutical composition provided herein.

In another aspect, the present disclosure provides methods of inhibiting the aberrantly high activity and/or production of a GSK3 in a subject in need thereof, cell, tissue, or biological sample, the method comprising administering to the subject or contacting the cell, tissue, or biological sample with an effective amount of a compound or pharmaceutical composition provided herein.

In another aspect, the present disclosure provides methods of inhibiting the aberrantly high activity and/or production of a GSK3 in a cell, tissue, or biological sample, the method comprising contacting the cell, tissue, or biological sample with an effective amount of a compound or pharmaceutical composition provided herein, wherein the cell, tissue, or biological sample is in vitro.

The details of one or more embodiments of the disclosure are set forth herein. Other features, objects, and advantages of the disclosure will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

The phrases “in certain embodiments” and “in some embodiments” are used interchangeably.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms (e.g., enriched 3-, 10-, 30-, 100-, 300-, 1,000-, 3,000- or 10,000-fold above their natural abundance). For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. In certain embodiments, an isotopically labeled compound comprises one or more deuterium. In certain embodiments, an isotopically labeled compound comprises one or more ¹³C. In certain embodiments, an isotopically labeled compound comprises one or more ¹⁵N or ¹⁸O. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). In some embodiments, the one or more carbon-carbon double bonds is internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In some embodiments, in an alkenyl group, a C═C double bond for which the stereochemistry is not specified (e.g., —CH═CHCH₃ or

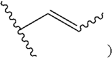

is in the (F)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₂₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₅ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). In some embodiments, the one or more carbon-carbon triple bonds is internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and for 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₀ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₄ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and is saturated or contains one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In some embodiments, in heterocyclyl groups that contain one or more nitrogen atoms, the point of attachment is a carbon or nitrogen atom, as valency permits. In some embodiments, a heterocyclyl group is either monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and is either saturated or contains one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as l-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In some embodiments, in heteroaryl groups that contain one or more nitrogen atoms, the point of attachment is a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. In some embodiments, in polycyclic heteroaryl groups wherein one ring does not contain a heteroatom (e.g., indolyl, quinolinyl, carbazolyl, and the like) the point of attachment is on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂—R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R ee, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁸ groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁-6 alkyl), —C(═S)SC₁₋₆ alkyl, —SC(═S)SCI-s alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl. C₃₋₁₀ carbocyclyl, C₆₋₁₀ aryl, 3-10 membered heterocyclyl, or 5-10 membered heteroaryl; or two geminal R^{gg} substituents are joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁-10 alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{aa}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein. The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. In some embodiments, the term “acyl” refers to a group having the general formula —C(═O)R^{aa}, —C(═O)OR^{aa}, —C(═O)—O—C(═O)R^{aa}, —C(═O)SR^{aa}, or —C(═O)N(R^{bb})₂.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

Nitrogen atoms are substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, —P(═O)(OR^{cc})₂, —P(═O)(R^{aa})₂, —P(═O)N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{d} groups, and wherein R^{aa}, R^{bb}, R^{cc}, and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₂₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₁₀ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, r-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxy benzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives. N-benzovlphenylalanyl derivatives. N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine. N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), i-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O))R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. In some embodiments, an anionic counterion is monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆, AsFb⁻, SbFb⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. In some embodiments, the compounds provided herein are prepared, e.g., in crystalline form. In some embodiments, the compounds provided herein are prepared, e.g., in crystalline form, and are solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound, or a salt thereof, that is associated with water. Typically, the number of the water molecules contained in a hydrate of a compound, or a salt thereof, is in a definite ratio to the number of the compound molecules in the hydrate. Therefore, in some embodiments, a hydrate of a compound, or a salt thereof, is represented, for example, by the general formula R·x H₂O, wherein R is the compound, or a salt thereof, and x is a number greater than 0. A given compound, or a salt thereof, may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. In some embodiments, an enantiomer is characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. In some embodiments, co-crystals are useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. In some embodiments, various polymorphs of a compound (or a salt, hydrate, or solvate thereof) are prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. In some embodiments. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein are preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal is a male or female at any stage of development. In some embodiments, the non-human animal is a transgenic animal or genetically engineered animal. The term “patient” refers to a human subject in need of treatment of a disease.

The term “tissue” refers to any biological tissue of a subject (including a group of cells, a body part, or an organ) or a part thereof, including blood and/or lymph vessels. In some embodiments, “tissue” is the object to which a compound, particle, and/or composition of the disclosure is delivered. In some embodiments, a tissue is an abnormal or unhealthy tissue, which may need to be treated. A tissue may also be a normal or healthy tissue that is under a higher than normal risk of becoming abnormal or unhealthy, which may need to be prevented.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “kinase” represents transferase class enzymes that are able to transfer a phosphate group from a donor molecule to an acceptor molecule, e.g., an amino acid residue of a protein or a lipid molecule. Representative, non-limiting examples of kinases include Abl, ACK, Akt1/PKBα, Akt2/PKBβ, Akt3/PKBγ, ALK1, ALK2, Alk4, AMPKα1/β1/γ1, AMPKα1/β1/γ2, AMPKα1/β1/γ3, AMPKα1/β2/γ1, AMPKα2/β1/γ1, AMPKα2/β2/γ2, Abl2, ARKS, Ask1, Aurora A, Aurora B, Aurora C, Axl, BARK1, Blk, Bmx, B-Raf, Brk, BrSKL, BrSK2, Btk, CaMK1α, CaMK1β, CaMK1γ, CaMK1δ, CAMK2α, CaMK2β, CAMK2δ, CAMK2γ, CAMK4, CAMKK1, CAMKK2, CDK1, CDK2, CDK3, CDK4, CDK5, CDK6, CDK7, CDK9, CDK1/cyclin B, CDK2/cyclin A, CDK2/cyclin E, CDK3/cyclin E, CDK5/p25, CDK5/p35, CDK6/cyclinD3, CDK7/cyclin H/MAT1, CDK9/cyclin T1, CHK1, CHK2, CK1α, CK1γ, CK1δ, CK1ε, CK1β1, CK1γ1, CK1γ2, CK1γ3, CK2α1, CK2α2, cKit, c-RAF, CLK1, CLK2, CLK3, COT, Csk, DAPK1, DAPK2, DAPK3, DCAMLK2, DDR2, DMPK, DRAK1, DYRKIA, DYRK2, DYRK3, eEF2K, EGFR, EPHA1, EPHA2, EPHA3, EPHA4, EPHA5, EPHA6, EPHA7, EPHA8, EphB1, EphB2, EphB3, EphB4, ErbB4, Erk1, Erk2, FAK, Fer, Fes, FGFR1, Flt2, Flt4, FLT3 D835Y, FGFR2, FGFR3, FGFR4, Fgr, Fit1, Flt3, Fms, FRK, FynA, GCK, GPRK5, GRK2, GRK4, GRK6, GRK7, GSK3α, GSK3β, Hck, HER2, HER4, HIPK1, HIPK2, HIPK3, HIPK4, IGFIR, IKKβ, IKKα, IKKε, IR, InsR, IRR, IRAK1, IRAK2, IRAK4, Itk, JAK2, JAK3, JNK1, JNK2, JNK3, KDR, KHS1, Kit, Lck, LIMK1, LKB1, LOK, LRRK2, Lyn A, Lyn B, MAPK1, MAPK2, MAPK12, MAPKAP-K2, MAPKAP-K3, MAPKAPK2, MAPKAPK3, MAPKAPK5, MARK1, MARK2, MARK3, MARK4, MELK, MEK1, MEK2, MEKK2, MEKK3, Mer, Met, MET M1250T, MINK, MKK4, MKK6, MKK7β, MLCK, MLK1, MLK3, MNK1, MNK2, MRCKα, MRCKβ, MSK1, MSK2, MSSK1, STK23, STK4, STK3, STK24, MST1, MST2, MST3, MST4, MUSK, mTOR, MYO3β, MYT1, NDR1, NEK11, NEK2, NEK3, NEK6, NEK7, NEK9, NLK, NUAK2, p38α, p38β, p38δ, p38γ, p70S6K, S6K, SRK, PAK1/CDC42, PAK2, PAK3, PAK4, PAK5, PAK6, PAR-1Bα, PASK, PBK, PDGFRα, PDGFRβ, PDK1, PEK, PHKG2, PI3Kα, PI3Kβ, PI3Kγ, PI3Kδ, Pim1, Pim2, PKAcα, PKAcβ, PKAcγ, PKA(b), PKA, PKBα, PKBβ, PKBγ, PKCα, PKCβ1, PKCβ2, PKCβ11, PKCδ, PKCε, PKCγ, PKCμ, PKCη, PKCt, PKCθ, PKCζ, PKD1, PKD2, PKD3, PKG1α, PKG1B, PKN1, PKN2, PKR, PLK1, PLK2, PLK3, PLK4, Polo, PRAK, PRK2, PrKX, PTK5, PYK2, QIK, Raf1, Ret, RIPK2, RIPK5, ROCK1, ROCK2, RON, ROS, Rse, RSK1, RSK2, RSK3, RSK4, SAPK2a, SAPK2b, SAPK3, SAPK4, SGK1, SGK2, SGK3, SIK, MLCK, SLK, Snk, Src, SRPK1, SRPK2, STK33, SYK, TAK1-TAB1, TAKI, TBK1, TAO1, TAO2, TAO3, TBK1, TEC, TESK1, TGFβR1, TGFβR2, Tie2, TLK2, TrkA, TrkB, TrkC, TSSK1, TSSK2, TTK, TXK, TYK2, TYRO3, ULK1, ULK2, WEE1, WNK2, WNK3, Yes1, YSK1, ZAK, ZAP70, ZC3, and ZIPK.

The term “administer,” “administering,” or “administration” refers to implanting, absorbing, ingesting, injecting, inhaling, or otherwise introducing a compound provided herein, a compound useful in a provided method, or a pharmaceutical composition provided herein, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment is administered after one or more signs or symptoms of the disease have developed or have been observed. In other embodiments, treatment is administered in the absence of signs or symptoms of the disease. For example, in some embodiments, treatment is administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting the activity and/or production of a GSK3. In certain embodiments, a therapeutically effective amount is an amount sufficient for treating a disease. In certain embodiments, a therapeutically effective amount is an amount sufficient for inhibiting the activity and/or production of a GSK3 and treating a disease.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting the activity and/or production of a GSK3. In certain embodiments, a prophylactically effective amount is an amount sufficient for preventing a disease. In certain embodiments, a prophylactically effective amount is an amount sufficient for inhibiting the activity and/or production of a GSK3 and preventing a disease.

The term “inhibit” or “inhibition”, for example, in the context of a GSK3, refers to a reduction in activity or production. In some embodiments, the term refers to a reduction of the level of activity and/or production, e.g., GSK3 activity and/or production, to a level that is statistically significantly lower than an initial level, which may, for example, be a baseline level of activity and/or production. In some embodiments, the term refers to a reduction of the level of activity and/or production, e.g., GSK3 activity and/or production, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of activity and/or production.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adrenoleukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer. Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher's disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC). Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease. Noonan syndrome, obesity, ovarian cancer, pancreatic cancer, Parkinson's disease, paroxysmal nocturnal hemoglobinuria. Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The cancer may be a solid tumor. The cancer may be a hematological malignancy. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, dermatitis (e.g., stasis dermatitis, allergic contact dermatitis, atopic dermatitis, irritant contact dermatitis, neurodermatitis perioral dermatitis, seborrheic dermatitis), hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, inflammatory rosacea. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease. Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Amold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor, Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “metabolic disease” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

The term “psychiatric disorder” refers to a condition or disorder relating to the functioning of the brain and the cognitive processes or behavior. Psychiatric disorders may be further classified based on the type of neurological disturbance affecting the mental faculties. Psychiatric disorders are expressed primarily in abnormalities of thought, feeling, emotion, and/or behavior producing either distress or impairment of function (for example, impairment of mental function such with dementia or senility). The term “psychiatric disorder” is, accordingly, sometimes used interchangeably with the term “mental disorder” or the term “mental illness”.

A psychiatric disorder is often characterized by a psychological or behavioral pattern that occurs in an individual and is thought to cause distress or disability that is not expected as part of normal development or culture. Definitions, assessments, and classifications of mental disorders can vary, but guideline criteria listed in the International Classification of Diseases and Related Health Problems (ICD, published by the World Health Organization, WHO), or the Diagnostic and Statistical Manual of Mental Disorders (DSM, published by the American Psychiatric Association, APA) and other manuals are widely accepted by mental health professionals. Individuals may be evaluated for various psychiatric disorders using criteria set forth in these and other publications accepted by medical practitioners in the field and the manifestation and severity of a psychiatric disorder may be determined in an individual using these publications.

Categories of diagnoses in these schemes may include dissociative disorders, mood disorders, anxiety disorders, psychotic disorders, eating disorders, developmental disorders, personality disorders, and other categories. There are different categories of mental disorder, and many different facets of human behavior and personality that can become disordered.

One group of psychiatric disorders includes disorders of thinking and cognition, such as schizophrenia and delirium. A second group of psychiatric disorders includes disorders of mood, such as affective disorders and anxiety. A third group of psychiatric disorders includes disorders of social behavior, such as character defects and personality disorders. And a fourth group of psychiatric disorders includes disorders of learning, memory, and intelligence, such as mental retardation and dementia. Accordingly, psychiatric disorders encompass schizophrenia, delirium, attention deficit disorder (ADD), schizoaffective disorder, depression (e.g., lithium-resistant depression), mania, attention deficit disorders, drug addiction, dementia, agitation, apathy, anxiety, psychoses, personality disorders, bipolar disorders, unipolar affective disorder, obsessive-compulsive disorders, eating disorders, post-traumatic stress disorders, irritability, adolescent conduct disorder, and disinhibition.

Some diseases classified as neurodegenerative diseases, for example Alzheimer's disease, also sometimes show aspects of psychiatric disorders as listed herein, for example disorders of memory or dementia. Some neurodegenerative diseases or manifestations thereof can, accordingly, also be referred to as psychiatric disorders. These terms are, therefore, not mutually exclusive.

The state of anxiety or fear can become disordered, so that it is unusually intense or generalized over a prolonged period of time. Commonly recognized categories of anxiety disorders include specific phobia, generalized anxiety disorder, social anxiety disorder, panic disorder, agoraphobia, obsessive-compulsive disorder, post-traumatic stress disorder.

Relatively long-lasting affective states can also become disordered. Mood disorder involving unusually intense and sustained sadness, melancholia or despair is known as clinical depression (or major depression), and may more generally be described as emotional dysregulation. Milder but prolonged depression can be diagnosed as dysthymia. Bipolar disorder involves abnormally “high” or pressured mood states, known as mania or hypomania, alternating with normal or depressed mood.

Patterns of belief, language use and perception can become disordered. Psychotic disorders centrally involving this domain include schizophrenia and delusional disorder, schizoaffective disorder is a category used for individuals showing aspects of both schizophrenia and affective disorders. Schizotypy is a category used for individuals showing some of the traits associated with schizophrenia but without meeting cut-off criteria.

The fundamental characteristics of a person that influence his or her cognitions, motivations, and behaviors across situations and time—can be seen as disordered due to being abnormally rigid and maladaptive. Categorical schemes list a number of different personality disorders, such as those classed as eccentric (e.g., paranoid personality disorder, schizoid personality disorder, schizotypal personality disorder), those described as dramatic or emotional (antisocial personality disorder, Borderline personality disorder, histrionic personality disorder, narcissistic personality disorder) or those seen as fear-related (avoidant personality disorder, dependent personality disorder, obsessive-compulsive personality disorder).

### BRIEF DESCRIPTION OF THE DRAWINGS

FIG. 1 shows a co-crystal structure of human GSK3β and Compound 133A. FIG. 1 indicates that Compound 133A is the same as Compound 133S.

### DETAILED DESCRIPTION OF CERTAIN EMBODIMENTS

The present disclosure provides compounds of Formula I, pharmaceutically acceptable salts, solvates, hydrates, polymorphs, co-crystals, tautomers, stereoisomers, isotopically labeled compounds, and prodrugs thereof, and pharmaceutical compositions and kits thereof. Also provided herein are methods of treating and/or preventing a disease in a subject in need thereof, as well as methods of inhibiting the activity and/or production GSK3 in a subject in need thereof, cell, tissue, or biological sample in vivo or in vitro.

### Compounds

In one aspect, provided herein is a compound, or a pharmaceutically acceptable salt, solvate, hydrate, polymorph, co-crystal, tautomer, stereoisomer, isotopically labeled compound, or prodrug thereof, wherein the compound is of Formula I:

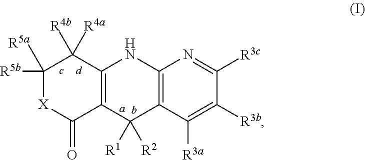

wherein:
R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R² is optionally substituted alkyl, optionally substituted alkenyl, or optionally substituted alkynyl;
or R¹ and R² are taken together with their intervening atom to form an optionally substituted, monocyclic, carbocyclic or heterocyclic ring, which is optionally fused to an optionally substituted, aryl, heteroaryl, carbocyclic, or heterocyclic ring, and/or optionally forms a spiro linkage with an optionally substituted, carbocyclic or heterocyclic ring; each of R^{3a} and R^{3b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R A)₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃;
or R^{3a} and R^{3b} are taken together with their intervening atoms to form an optionally substituted, monocyclic, carbocyclic, heterocyclic, or heteroaryl ring, or an optionally substituted phenyl ring;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{3c} is hydrogen;
X is

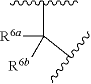

or —N(R⁶)—;
each of R^{4a}, R^{4b}, R^{5a}, R^{5b}, R^{6a}, and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
or R^{4a} and R^{4b} are taken together with their intervening atom to form an optionally substituted, monocyclic, carbocyclic or heterocyclic ring;
or R^{4b} and R^{5a} are taken together with their intervening atoms to form an optionally substituted, monocyclic, carbocyclic or heterocyclic ring;
or R^{5a} and R^{5b} are taken together with their intervening atom to form an optionally substituted, monocyclic, carbocyclic or heterocyclic ring;
or R^{5b} and R^{6a} are taken together with their intervening atoms to form an optionally substituted, monocyclic, carbocyclic or heterocyclic ring;
or R^{6a} and R^{6b} are taken together with their intervening atom to form an optionally substituted, monocyclic, carbocyclic or heterocyclic ring; and
R⁶ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In some embodiments,

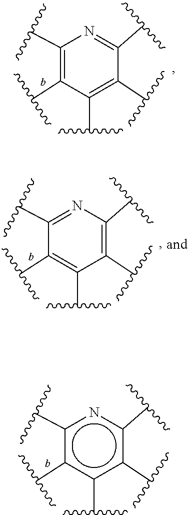

are used interchangeably.

In some embodiments, R¹ is optionally substituted C₁₋₁₀ alkyl, optionally substituted C₂₋₁₀ alkenyl, or optionally substituted C₂₋₁₀ alkynyl.

In some embodiments, R¹ is optionally substituted carbocyclyl or optionally substituted heterocyclyl. In some embodiments, R¹ is optionally substituted C₃₋₁₄ carbocyclyl. In some embodiments, R¹ is optionally substituted monocyclic C₃₋₇ carbocyclyl. In some embodiments, R¹ is optionally substituted monocyclic C₃₋₄ carbocyclyl. In some embodiments, R¹ is optionally substituted monocyclic C₅₋₇ carbocyclyl. In some embodiments, R¹ is saturated carbocyclyl. In some embodiments, R¹ is carbocyclyl comprising only one unsaturated bond (e.g., C═C bond) in the carbocyclic ring system.

In some embodiments, R¹ is 3- to 14-membered optionally substituted heterocyclyl. In some embodiments, R¹ is optionally substituted monocyclic 3- to 7-membered heterocyclyl. In some embodiments, R¹ is optionally substituted 3- to 14-membered heterocyclyl comprising one or more O and/or S atoms but no N atoms. In some embodiments, R¹ is optionally substituted 3-to 14-membered heterocyclyl comprising one or more N atoms and optionally one or more O and/S atoms. In some embodiments, R¹ is optionally substituted monocyclic 3- to 7-membered heterocyclyl comprising one or more O and/or S atoms but no N atoms. In some embodiments, R¹ is optionally substituted monocyclic 3- to 7-membered heterocyclyl comprising one or more N atoms and optionally one or more O and/or S atoms. In some embodiments, R¹ is saturated heterocyclyl. In some embodiments, R¹ is heterocyclyl comprising only one unsaturated bond (e.g., C═C bond) in the heterocyclic ring system.

In some embodiments, R¹ is optionally substituted aryl. In some embodiments, R¹ is optionally substituted monocyclic aryl. In some embodiments, R¹ is optionally substituted bicyclic aryl. In some embodiments, R¹ is optionally substituted C₆₋₁₄ aryl. In some embodiments, R¹ is optionally substituted C₆₋₁₀ aryl. In some embodiments, R¹ is optionally substituted phenyl. In some embodiments, R¹ is unsubstituted phenyl. In some embodiments, R¹ is substituted phenyl. In some embodiments, R¹ is ortho mono-substituted phenyl. In some embodiments, R¹ is meta mono-substituted phenyl. In some embodiments, R¹ is para mono-substituted phenyl. In some embodiments, R¹ is di-substituted phenyl. In some embodiments, R¹ is tri-substituted phenyl. In some embodiments, R¹ is optionally substituted naphthyl.

In some embodiments, R¹ is optionally substituted heteroaryl. In some embodiments, R¹ is optionally substituted monocyclic heteroaryl. In some embodiments, R¹ is optionally substituted bicyclic heteroaryl. In some embodiments, R¹ is optionally substituted 5- to 14-membered heteroaryl. In some embodiments, R¹ is optionally substituted 5- to 10-membered heteroaryl. In some embodiments, R¹ is optionally substituted 5- to 6-membered monocyclic heteroaryl. In some embodiments, R¹ is optionally substituted, 2-, 3-, or 4-pyridinyl. In some embodiments, R¹ is mono-substituted, 2-, 3-, or 4-pyridinyl. In some embodiments, R¹ is meta mono-substituted, 2- or 4-pyridinyl. In some embodiments, R¹ is optionally substituted 9- to 10-membered bicyclic heteroaryl.

In some embodiments, R¹ is optionally substituted aryl fused with optionally substituted monocyclic C₃₋₇ carbocyclyl. In some embodiments, R¹ is optionally substituted C₆₋₁₄ aryl fused with optionally substituted monocyclic C₃₋₇ carbocyclyl. In some embodiments, R¹ is optionally substituted C₆₋₁₀ aryl fused with optionally substituted monocyclic C₃₋₇ carbocyclyl. In some embodiments, R¹ is optionally substituted phenyl fused with optionally substituted monocyclic C₃₋₇ carbocyclyl. In some embodiments, R¹ is optionally substituted naphthyl fused with optionally substituted monocyclic C₃₋₇ carbocyclyl.

In some embodiments, R¹ is optionally substituted aryl fused with optionally substituted monocyclic 3- to 7-membered heterocyclyl. In some embodiments, R¹ is optionally substituted C₆₋₁₄ aryl fused with optionally substituted monocyclic 3- to 7-membered heterocyclyl. In some embodiments, R¹ is optionally substituted C₆₋₁₀ aryl fused with optionally substituted monocyclic 3- to 7-membered heterocyclyl. In some embodiments, R¹ is optionally substituted phenyl fused with optionally substituted monocyclic 3- to 7-membered heterocyclyl. In some embodiments, R¹ is optionally substituted naphthyl fused with optionally substituted monocyclic 3- to 7-membered heterocyclyl.

In some embodiments, R¹ is optionally substituted heteroaryl fused with optionally substituted monocyclic C₃₋₇ carbocyclyl. In some embodiments, R¹ is optionally substituted 5- to 14-membered heteroaryl fused with optionally substituted monocyclic C₃₋₇ carbocyclyl. In some embodiments, R¹ is optionally substituted 5- to 10-membered heteroaryl fused with optionally substituted monocyclic C₃₋₇ carbocyclyl. In some embodiments, R¹ is optionally substituted 5- to 6-membered monocyclic heteroaryl fused with optionally substituted monocyclic C₃₋₇ carbocyclyl. In some embodiments, R¹ is optionally substituted 9- to 10-membered bicyclic heteroaryl fused with optionally substituted monocyclic C₃₋₇ carbocyclyl.

In some embodiments, R¹ is optionally substituted heteroaryl fused with optionally substituted monocyclic 3- to 7-membered heterocyclyl. In some embodiments, R¹ is optionally substituted 5- to 14-membered heteroaryl fused with optionally substituted monocyclic 3- to 7-membered heterocyclyl. In some embodiments, R¹ is optionally substituted 5- to 10-membered heteroaryl fused with optionally substituted monocyclic 3- to 7-membered heterocyclyl. In some embodiments, R¹ is optionally substituted 5- to 6-membered monocyclic heteroaryl fused with optionally substituted monocyclic 3- to 7-membered heterocyclyl. In some embodiments, R¹ is optionally substituted 9- to 10-membered bicyclic heteroaryl fused with optionally substituted monocyclic 3- to 7-membered heterocyclyl.

In some embodiments, R¹ is

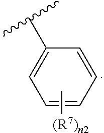

In certain embodiments, (i) each instance of R⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —S(═O)(═NR^{D})R^{D}, —S(═O)(═NR^{D})OR^{D}, —S(═O)(═NR^{D})SR^{D}, —S(═O)(═NR^{D})N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —OS(═O)(═NR^{D})R^{D}, —OS(═O)(═NR^{D})OR^{D}, —OS(═O)(═NR^{D})SR^{D}, —OS(═O)(═NR^{D})N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(RDN, —NR^{D}C(═NR^{D})R^{D}, —NR^{D}C(═NR^{D})OR^{D}, —NR^{D}C(═NRW)SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —NR^{D}S(═O)(═NR^{D})R^{D}, —NR^{D}S(═O)(═NR^{D})OR^{D}, —NR^{D}S(═O)(═NR^{D})NR^{D}SR^{D}, —NR^{D}S(═O)(═NR^{D})N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃; or
(ii) two instances of R⁷ on two adjacent carbon atoms are taken together with the two adjacent carbon atoms to form an optionally substituted, monocyclic, aryl, heteroaryl, carbocyclic, or heterocyclic ring, and the remaining instances of R⁷, if present, are as defined in (i).

n2 is 0, 1, 2, 3, 4, or 5. In some embodiments, n2 is 0, 1, 2, 3, or 4. In some embodiments, n2 is 0, 1, 2, or 3. In some embodiments, n2 is 0, 1, or 2. In some embodiments, n2 is 0 or 1. In some embodiments, n2 is 0. In some embodiments, n2 is 1. In some embodiments, n2 is 2. In some embodiments, n2 is 3. In some embodiments, n2 is 4. In some embodiments, n2 is 5.

In some embodiments, R¹ is

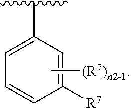

In some embodiments, R¹ is

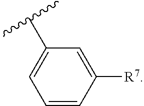

In some embodiments, R¹ is

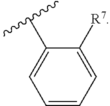

In some embodiments, R¹ is

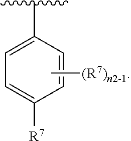

In some embodiments, R¹ is

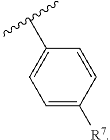

In some embodiments, R¹ is

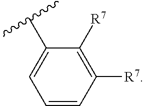

In some embodiments, R¹ is

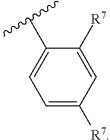

In some embodiments, R¹ is

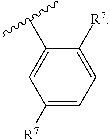

In some embodiments, R¹ is

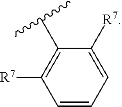

In some embodiments, R¹ is

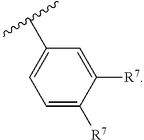

In some embodiments, R¹ is

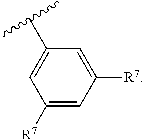

In some embodiments, at least one instance of R⁷ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, at least one instance of R⁷ is optionally substituted C₁₋₁₀ alkyl, optionally substituted C₂₋₁₀ alkenyl, optionally substituted C₂₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, at least one instance of R⁷ is optionally substituted alkyl. In some embodiments, at least one instance of R⁷ is unsubstituted C₁₋₆ alkyl or C₁₋₆ alkyl substituted with one or more halogen, as valency permits. In some embodiments, at least one instance of R⁷ is C₁₋₁₀ haloalkyl. In some embodiments, at least one instance of R⁷ is C₁₋₄ haloalkyl. In some embodiments, at least one instance of R⁷ is C₁₋₄ fluoroalkyl (e.g., C₁₋₄ perfluoroalkyl). In some embodiments, at least one instance of R⁷ is —CF₃.

In some embodiments, at least one instance of R⁷ is halogen. In some embodiments, at least one instance of R⁷ is bromine, chlorine, or fluorine. In some embodiments, at least one instance of R⁷ is bromine or chlorine. In some embodiments, at least one instance of R⁷ is chlorine or fluorine. In some embodiments, at least one instance of R⁷ is bromine. In some embodiments, at least one instance of R⁷ is chlorine. In some embodiments, at least one instance of R⁷ is fluorine.

In some embodiments, at least one instance of R⁷ is —OR^{D}, —SR^{D}, or —N(R^{D})₂ (e.g., wherein R^{D} is hydrogen, optionally substituted alkyl, or optionally substituted phenyl). In some embodiments, at least one instance of R⁷ is —OH. In some embodiments, at least one instance of R⁷ is —SR^{D}. In some embodiments, at least one instance of R⁷ is —S(optionally substituted alkyl).

In some embodiments, at least one instance of R⁷ is —S(unsubstituted C₁₋₆ alkyl or C₁₋₆ alkyl substituted with one or more halogen). In some embodiments, at least one instance of R⁷ is —SMe or —SCF₃. In some embodiments, at least one instance of R⁷ is —SH. In some embodiments, at least one instance of R⁷ is —NH₂. In some embodiments, at least one instance of R⁷ is —CN, —SCN, —SSR^{D}, —N₃, —NO, or —NO₂. In some embodiments, at least one instance of R⁷ is —C(═O)R^{D}, —C(═O)OR^{D}, —C(═O)SR^{D}, or —C(═O)N(R^{D})₂ (e.g., wherein R^{D} is hydrogen or optionally substituted alkyl, or optionally substituted phenyl). In some embodiments, at least one instance of R⁷ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, at least one instance of R⁷ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, at least one instance of R⁷ is —S(═O)₂R^{D}. In some embodiments, at least one instance of R⁷ is —S(═O)₂(optionally substituted alkyl). In some embodiments, at least one instance of R⁷ is —S(═O)₂(unsubstituted C₁₋₆ alkyl or C₁₋₆ alkyl substituted with one or more halogen). In some embodiments, at least one instance of R⁷ is —S(═O)₂Me or —S(═O)₂CF₃. In some embodiments, at least one instance of R⁷ is —S(═O)(═NR^{D})R^{D}. In some embodiments, at least one instance of R⁷ is —S(═O)(═NR^{D})(optionally substituted alkyl). In some embodiments, at least one instance of R⁷ is —S(═O)(═NR^{D})(unsubstituted C₁₋₆ alkyl or C₁₋₆ alkyl substituted with one or more halogen). In some embodiments, at least one instance of R⁷ is —S(═O)(═NR^{D})Me. In some embodiments, at least one instance of R⁷ is —S(═O)(═NH)(optionally substituted alkyl). In some embodiments, at least one instance of R⁷ is —S(═O)(═NH)(unsubstituted C₁₋₆ alkyl or C₁₋₆ alkyl substituted with one or more halogen). In some embodiments, at least one instance of R⁷ is —S(═O)(═NH)Me. In some embodiments, at least one instance of R⁷ is —S(═O)(═N(unsubstituted C₁₋₆ alkyl))(optionally substituted alkyl). In some embodiments, at least one instance of R⁷ is —S(═O)(═N(unsubstituted C₁₋₆ alkyl))(unsubstituted C₁₋₆ alkyl or C₁₋₆ alkyl substituted with one or more halogen). In some embodiments, at least one instance of R⁷ is —S(═O)(═N(unsubstituted C₁₋₆ alkyl))Me (e.g., —S(═O)(═NMe)Me). In some embodiments, at least one instance of R⁷ is —OC(═O)R^{D}, —C(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, or —ON(R^{D})₂ (e.g., wherein R^{D} is hydrogen or optionally substituted alkyl, or optionally substituted phenyl). In some embodiments, at least one instance of R⁷ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D})₂ (e.g., wherein R^{D} is hydrogen or optionally substituted alkyl, or optionally substituted phenyl). In some embodiments, at least one instance of R⁷ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, at least one instance of R⁷ is —Si(R^{D})₃, —Si(R^{D})₂OR^{D}, —Si(R^{D})(OR^{D})₃, —Si(OR^{D})₃, —OSi(R¹)₃, —OSi(R^{D})₂OR^{D}, —OSi(R^{D})(OR^{D})₂, or —OSi(OR^{D})₃ (e.g., wherein R^{D} is hydrogen or optionally substituted alkyl, or optionally substituted phenyl).

In some embodiments, at least one instance of R⁷ is optionally substituted carbocyclyl, optionally substituted heterocyclyl, optionally substituted aryl, or optionally substituted heteroaryl. In some embodiments, at least one instance of R⁷ is optionally substituted C₃₋₁₄ carbocyclyl. In some embodiments, at least one instance of R⁷ is optionally substituted monocyclic C₃₋₇ carbocyclyl. In some embodiments, at least one instance of R⁷ is optionally substituted monocyclic C₃₋₄ carbocyclyl. In some embodiments, at least one instance of R⁷ is optionally substituted cyclobutyl. In some embodiments, at least one instance of R⁷ is optionally substituted monocyclic C₅₋₇ carbocyclyl. In some embodiments, at least one instance of R⁷ is saturated carbocyclyl. In some embodiments, at least one instance of R⁷ is carbocyclyl comprising only one unsaturated bond (e.g., C═C bond) in the carbocyclic ring system.

In some embodiments, at least one instance of R⁷ is optionally substituted 3- to 14-membered heterocyclyl. In some embodiments, at least one instance of R⁷ is optionally substituted monocyclic 3- to 7-membered heterocyclyl. In some embodiments, at least one instance of R⁷ is optionally substituted 3- to 14-membered heterocyclyl comprising one or more O and/or S atoms but no N atoms. In some embodiments, at least one instance of R⁷ is optionally substituted 3- to 14-membered heterocyclyl comprising one or more N atoms and optionally one or more O and/S atoms. In some embodiments, at least one instance of R⁷ is optionally substituted monocyclic 3- to 7-membered heterocyclyl comprising one or more O and/or S atoms but no N atoms. In some embodiments, at least one instance of R⁷ is optionally substituted monocyclic 3- to 7-membered heterocyclyl comprising one or more N atoms and optionally one or more O and/or S atoms. In some embodiments, at least one instance of R⁷ is saturated heterocyclyl. In some embodiments, at least one instance of R⁷ is heterocyclyl comprising only one unsaturated bond (e.g., C═C bond) in the heterocyclic ring system.

In some embodiments, at least one instance of R⁷ is optionally substituted monocyclic aryl. In some embodiments, at least one instance of R⁷ is optionally substituted bicyclic aryl. In some embodiments, at least one instance of R⁷ is optionally substituted C₆₋₁₄ aryl. In some embodiments, at least one instance of R⁷ is optionally substituted C₆₋₁₀ aryl. In some embodiments, at least one instance of R⁷ is optionally substituted phenyl. In some embodiments, at least one instance of R⁷ is optionally substituted naphthyl.

In some embodiments, at least one instance of R⁷ is optionally substituted monocyclic heteroaryl. In some embodiments, at least one instance of R⁷ is optionally substituted bicyclic heteroaryl. In some embodiments, at least one instance of R⁷ is optionally substituted 5- to 14-membered heteroaryl. In some embodiments, at least one instance of R⁷ is optionally substituted 5- to 10-membered heteroaryl. In some embodiments, at least one instance of R⁷ is optionally substituted 5- to 6-membered monocyclic heteroaryl. In some embodiments, at least one instance of R⁷ is optionally substituted heteroaryl comprising one or more nitrogen atoms in the heteroaryl ring system, as valency permits. In some embodiments, at least one instance of R⁷ is at least one instance of R⁷ is optionally substituted pyridinyl, optionally substituted pyridazinyl, optionally substituted pyrazolyl, or optionally substituted imidazolyl. In some embodiments, at least one instance of R⁷ is optionally substituted 2-pyridinyl, optionally substituted 3-pyridinyl, or optionally substituted 4-pyridinyl. In some embodiments, at least one instance of R⁷ is optionally substituted pyridazinyl. In some embodiments, at least one instance of R⁷ is optionally substituted imidazolyl. In some embodiments, at least one instance of R⁷ is optionally substituted pyrazolyl. In some embodiments, at least one instance of R⁷ is optionally substituted, 5- or 6-membered, monocyclic heteroaryl fused to an optionally substituted, monocyclic, carbocyclic, heterocyclic, aryl, or heteroaryl ring. In some embodiments, at least one instance of R⁷ is optionally substituted, 5- or 6-membered, monocyclic heteroaryl fused to an optionally substituted, 4- to 7-membered, monocyclic carbocyclic ring. In some embodiments, at least one instance of R⁷ is —S(optionally substituted alkyl), —S(═O)(═NH)(optionally substituted alkyl), —S(═O)(═N(unsubstituted C₁₋₆ alkyl))(optionally substituted alkyl), —S(═O)₂(optionally substituted alkyl), or —OH. In some embodiments, at least one instance of R⁷ is —CH(CH₃)₂, —CF₃, unsubstituted cyclobutyl, —SCH₃, —SCF₃, —S(═O)(═NH)(CH₃), —S(═O)(═NCH₃)(CH₃), —S(═O)₂CH₃, —S(═O)₂CF₃, or —OH. In some embodiments, R⁷ is not —S(═O)₂CH₃. In some embodiments, R⁷ is not —S(═O)₂CH₃ when

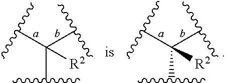

In some embodiments, R⁷ is not —S(═O)₂CH₃ when

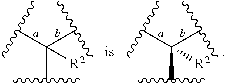

In some embodiments, two instances of R⁷ on two adjacent carbon atoms are taken together with the two adjacent carbon atoms to form an optionally substituted phenyl ring. In some embodiments, two instances of R⁷ on two adjacent carbon atoms are taken together with the two adjacent carbon atoms to form an optionally substituted, monocyclic, 5- or 6-membered heteroaryl ring. In some embodiments, two instances of R⁷ on two adjacent carbon atoms are taken together with the two adjacent carbon atoms to form an optionally substituted, monocyclic, 4- to 7-membered carbocyclic ring. In some embodiments, two instances of R⁷ on two adjacent carbon atoms are taken together with the two adjacent carbon atoms to form an optionally substituted, monocyclic, 5- to 7-membered heterocyclic ring.

Each instance of R^{D} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{D} attached to the same intervening atom are joined together with the intervening atom to form an optionally substituted, monocyclic, heterocyclic or heteroaryl ring. In some embodiments, at least one instance of R^{D} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{D} is optionally substituted carbocyclyl, optionally substituted heterocyclyl, optionally substituted aryl, or optionally substituted heteroaryl. In some embodiments, at least one instance of R^{D} is independently hydrogen, optionally substituted C₁₋₁₀ alkyl, optionally substituted C₂₋₁₀ alkenyl, optionally substituted C₂₋₁₀ alkynyl, optionally substituted C₃₋₁₄ carbocyclyl, or optionally substituted C₆₋₁₄ aryl. In some embodiments, at least one instance of R^{D} is independently hydrogen, optionally substituted C₁₋₁₀ alkyl, or optionally substituted phenyl. In some embodiments, at least one instance of R^{D} is a nitrogen protecting group when attached to a nitrogen atom, an oxygen protecting group when attached to an oxygen atom, or a sulfur protecting group when attached to a sulfur atom. In some embodiments, two instances of R^{D} attached to the same intervening atom are joined together with the intervening atom to form an optionally substituted, monocyclic, heterocyclic or heteroaryl ring.

In some embodiments, R² is optionally substituted alkyl, optionally substituted alkenyl, or optionally substituted alkynyl. In some embodiments, R² is optionally substituted C₁₋₄ alkyl, optionally substituted C₁₋₄ alkenyl, or optionally substituted C₁₋₄ alkynyl. In some embodiments, R² is optionally substituted alkyl. In some embodiments, R² is optionally substituted C₁₋₄ alkyl. In some embodiments, R² is unsubstituted C₁₋₄ alkyl or C₁₋₄ alkyl substituted with one or more fluorine, as valency permits. In some embodiments, R² is unsubstituted C₁-C₄ alkyl. In some embodiments, R² is —CH₃. In some embodiments, R² is —C₂H₅. In some embodiments, R² is —CH₂F, —CHF₂, or —CF₃. In some embodiments, R² is —CH₂CH₂F, —CH₂CHF₂, or —CH₂CF₃. In some embodiments, R¹ and R² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring. In certain embodiments, R¹ and R² are taken together with their intervening atom to form an optionally substituted, monocyclic, carbocyclic ring, which is optionally fused to an optionally substituted, aryl, heteroaryl, carbocyclic, or heterocyclic ring and/or optionally forms a spiro linkage with an optionally substituted, carbocyclic or heterocyclic ring. In some embodiments, R¹ and R² are taken together with their intervening atom to form an optionally substituted, monocyclic, carbocyclic ring. In some embodiments, R¹ and R² are taken together with their intervening atom to form an optionally substituted, monocyclic, 3- to 7-membered carbocyclic ring. In some embodiments, R¹ and R² are taken together with their intervening atom to form an optionally substituted, monocyclic, 5- to 6-membered carbocyclic ring. In some embodiments, R¹ and R² are taken together with their intervening atom to form an optionally substituted, monocyclic, heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring. In some embodiments, R¹ and R² are taken together with their intervening atom to form an optionally substituted, monocyclic, heterocyclic ring. In some embodiments, R¹ and R² are taken together with their intervening atom to form an optionally substituted, monocyclic, 3- to 7-membered heterocyclic ring. In some embodiments, R¹ and R² are taken together with their intervening atom to form an optionally substituted, monocyclic, 5- to 6-membered heterocyclic ring. In some embodiments, R¹ and R² are taken together with their intervening atom to form an optionally substituted, monocyclic, heterocyclic ring comprising one or more oxygen atoms. In some embodiments, R¹ and R² are taken together with their intervening atom to form an optionally substituted, monocyclic, 3- to 7-membered heterocyclic ring comprising one or more oxygen atoms. In some embodiments, R¹ and R² are taken together with their intervening atom to form an optionally substituted, monocyclic, heterocyclic ring comprising one or more nitrogen atoms. In some embodiments, R¹ and R² are taken together with their intervening atom to form an optionally substituted, monocyclic, 3- to 7-membered heterocyclic ring comprising one or more nitrogen atoms. In some embodiments, R¹ and R² are taken together with their intervening atom to form an optionally substituted, 5-membered, monocyclic, heterocyclic ring. In some embodiments, R¹ and R² are taken together with their intervening atom to form an optionally substituted, 6-membered, monocyclic, heterocyclic ring. In some embodiments, R¹ and R² are taken together with their intervening atom to form an optionally substituted, 5-membered, monocyclic, heterocyclic ring, which is fused to an optionally substituted phenyl ring. In some embodiments, R¹ and R² are taken together with their intervening atom to form an optionally substituted, 6-membered, monocyclic, heterocyclic ring, which is fused to an optionally substituted phenyl ring. In some embodiments, R¹ and R² are taken together with their intervening atom to form optionally substituted piperidinyl. In some embodiments, R¹ and R² are taken together with their intervening atom to form

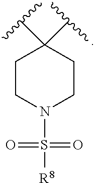

In some embodiments, R¹ and R² are taken together with their intervening atom to form

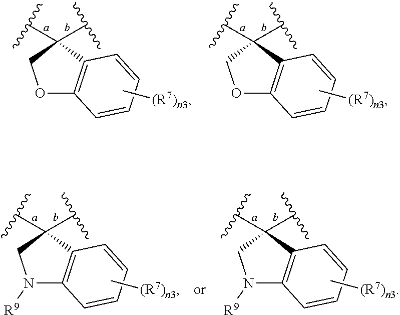

In some embodiments, R¹ and R² are taken together with their intervening atom to form

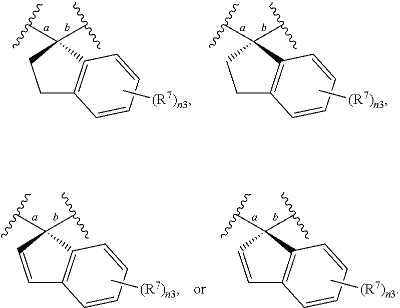

In some embodiments, R¹ and R² are taken together with their intervening atom to form

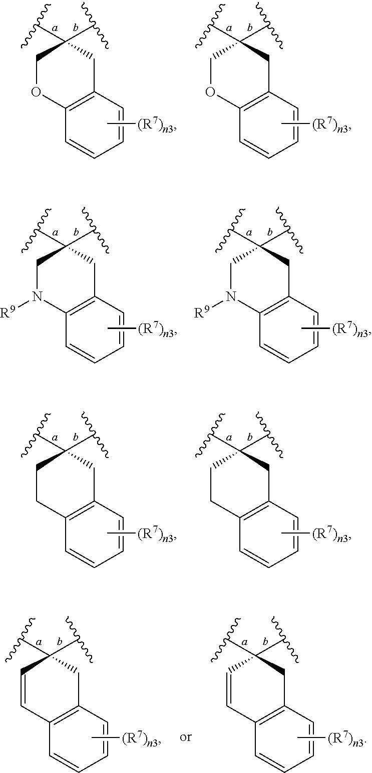

In some embodiments, R¹ and R² are taken together with their intervening atom to form

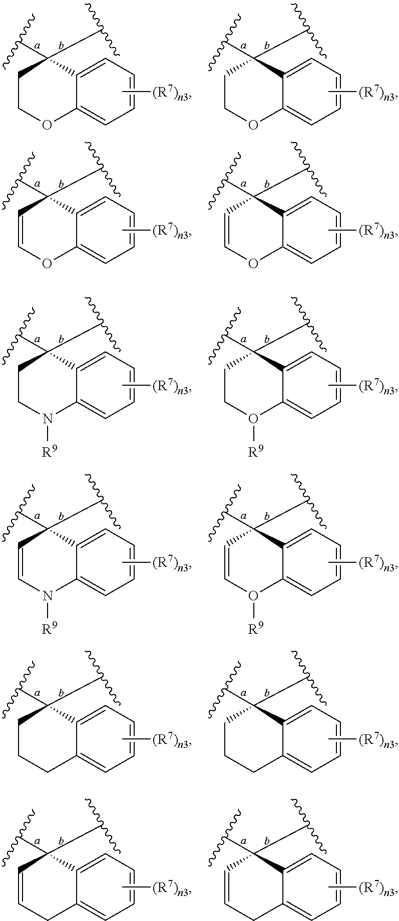

In certain embodiments,

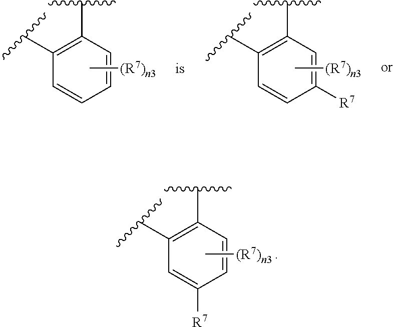

In certain embodiments,

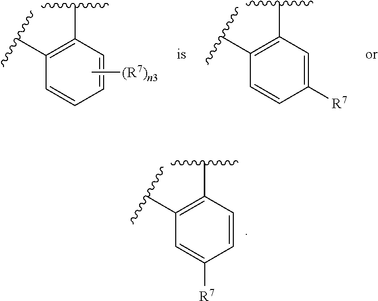

In certain embodiments,

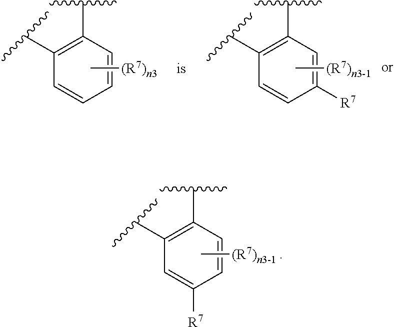

In certain embodiments,

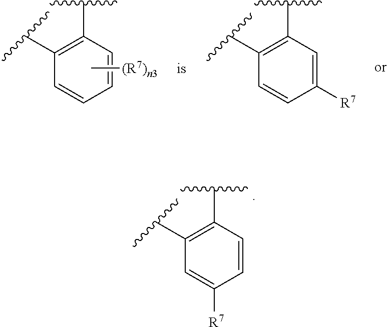

n3 is 0, 1, 2, 3, or 4. In some embodiments, n3 is 0. In some embodiments, n3 is 1. In some embodiments, n3 is 2. In some embodiments, n3 is 3. In some embodiments, n3 is 4.

R⁸ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a sulfur protecting group. In some embodiments, R⁸ is hydrogen, optionally substituted C₁₋₁₀ alkyl, optionally substituted C₂₋₁₀ alkenyl, optionally substituted C₂₋₁₀ alkynyl, optionally substituted C₁₋₁₀ heteroalkyl, optionally substituted C₁₋₁₀ heteroalkenyl, optionally substituted C₁₋₁₀ heteroalkynyl, optionally substituted C₃₋₁₄ carbocyclyl, optionally substituted 3- to 14-membered heterocyclyl, optionally substituted C₆₋₁₄ aryl, optionally substituted 5-14-membered heteroaryl, or a sulfur protecting group.

R⁹ is hydrogen, optionally substituted alkyl, or a nitrogen protecting group. In some embodiments, R⁹ is optionally substituted C₁₋₄ alkyl. In some embodiments, R⁹ is unsubstituted C₁₋₄ alkyl. In some embodiments, R⁹ is —CH₃. In some embodiments, R⁹ is —C₂H₅, —CH₂CH₂CH₃, or —CH(CH₃)₂. In certain embodiments, R⁹ is a nitrogen protecting group (e.g., Bn, Boc, Cbz, Fmoc, trifluoroacetyl, triphenylmethyl, acetyl, or Ts).

In some embodiments, is

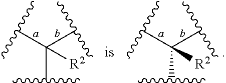

In some embodiments,

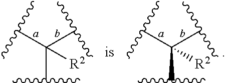

In some embodiments, R^{3a} is halogen, optionally substituted alkyl, optionally substituted alkenyl, optionally substituted alkynyl, optionally substituted heteroalkyl, optionally substituted heteroalkenyl, optionally substituted heteroalkynyl, —OR^{A}, —SR^{A}, or —N(R^{A})₂. In some embodiments, R is optionally substituted C₁₋₁₀ alkyl, optionally substituted C₂₋₁₀ alkenyl, optionally substituted C₂₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R^{3a} is C₁₋₁₀ haloalkyl. In some embodiments, R^{3a} is C₁₋₄ haloalkyl. In some embodiments, R^{3a} is C₁₋₄ fluoroalkyl (e.g., C₁₋₄ perfluoroalkyl). In some embodiments, R^{3a} is —CH₂F, —CHF₂, or —CF₃. In some embodiments, R^{3a} is —CF₃. In some embodiments, R^{3a} is —CH₂CH₂F, —CH₂CHF₂, or —CH₂CF₃. In some embodiments, R^{3a} is optionally substituted alkyl substituted with optionally substituted carbocyclyl. In some embodiments, R^{3a} is unsubstituted C₁₋₆ alkyl substituted with one optionally substituted C₃₋₇ monocyclic carbocyclyl. In some embodiments, R^{3a} is —CH₂-(unsubstituted cyclopropyl). In some embodiments, R^{3a} is hydrogen, optionally substituted C₁-C₆ alkyl, or halogen. In some embodiments, R^{3a} is hydrogen, fluorine, —CH₃, —CH₂F, —CHF₂, or —CF₃. In some embodiments, R^{3a} is hydrogen, fluorine, —CH₃, or —CF₃.

In some embodiments, R^{3a} is hydrogen or halogen. In some embodiments, R^{3a} is hydrogen or fluorine. In some embodiments, R^{3a} is hydrogen or optionally substituted C₁-C₆ alkyl. In some embodiments, R^{3a} is hydrogen, unsubstituted C₁-C₆ alkyl, or C₁₋₆ haloalkyl. In some embodiments, R^{3a} is —CH₃. In some embodiments, R^{3a} is —C₂H₅. In some embodiments, R^{3a} is —C₃H₇, —C₄H₉, or —C₅H₁₁. In some embodiments, R^{3a} is hydrogen. In some embodiments, R^{3a} is optionally substituted alkenyl (e.g., optionally substituted C₂₋₆ alkenyl). In some embodiments, R^{3a} is unsubstituted vinyl.

In some embodiments, R^{3a} is halogen. In some embodiments, R^{3a} is bromine, chlorine, or fluorine. In some embodiments, R^{3a} is bromine or chlorine. In some embodiments, R^{3a} is chlorine or fluorine. In some embodiments, R^{3a} is bromine. In some embodiments, R^{3a} is chlorine. In some embodiments, R^{3a} is fluorine.

In some embodiments, R^{3a} is —OR^{A}, —SR^{A}, or —N(R^{A}h (e.g., wherein R^{A} is hydrogen or optionally substituted alkyl, or optionally substituted phenyl). In some embodiments, R³ is —OH.

In some embodiments, R^{3a} is —SH. In some embodiments, R³ is —NH₂. In some embodiments, R^{3a} is —CN, —SCN, —SSR^{A}, —N₃, —NO, or —NO₂. In some embodiments, R^{3a} is —CN. In some embodiments, R^{3a} is —C(═O)R^{A}, —C(═O)OR^{A}, —C(═O)SR^{A}, or —C(═O)N(R^{A})₂ (e.g., wherein R^{A} is hydrogen or optionally substituted alkyl, or optionally substituted phenyl). In some embodiments. R^{3a} is —C(═NR^{A})R^{A}, —C(═NR^{A})OR^{A}, —C(═NR^{A})SR^{A}, or —C(═NR^{A})N(R^{A})₂ (e.g., wherein R^{A} is hydrogen or optionally substituted alkyl, or optionally substituted phenyl). In some embodiments, R^{3a} is —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, or —S(═O)₂N(R^{A})₂ (e.g., wherein R^{A} is hydrogen or optionally substituted alkyl, or optionally substituted phenyl). In some embodiments, R^{3a} is —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, or —ON(R^{A})₂ (e.g., wherein R^{A} is hydrogen or optionally substituted alkyl, or optionally substituted phenyl). In some embodiments, R³ is —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, or —SC(═NR^{A})N(R^{A})₂ (e.g., wherein R^{A} is hydrogen or optionally substituted alkyl, or optionally substituted phenyl). In some embodiments, R³ is —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, or —NR^{A}S(═O)₂N(R^{A})₂ (e.g., wherein R^{A} is hydrogen or optionally substituted alkyl, or optionally substituted phenyl). In some embodiments, R^{3a} is —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃ (e.g., wherein R^{A} is hydrogen or optionally substituted alkyl, or optionally substituted phenyl).

In some embodiments, R^{3a} is optionally substituted carbocyclyl, optionally substituted heterocyclyl, optionally substituted aryl, or optionally substituted heteroaryl. In some embodiments, R^{3a} is optionally substituted C₃₋₁₄ carbocyclyl. In some embodiments, R^{D} is optionally substituted monocyclic C₃₋₇ carbocyclyl. In some embodiments, R^{3a} is optionally substituted monocyclic C₃₋₄ carbocyclyl. In some embodiments, R^{3a} is optionally substituted cyclopropyl. In some embodiments, R^{3a} is optionally substituted monocyclic C₅₋₇ carbocyclyl. In some embodiments, R^{3a} is saturated carbocyclyl. In some embodiments, R^{3a} is carbocyclyl comprising only one unsaturated bond (e.g., C═C bond) in the carbocyclic ring system.

In some embodiments, R^{3a} is optionally substituted 3- to 14-membered heterocyclyl. In some embodiments, R^{3a} is optionally substituted monocyclic 3- to 7-membered heterocyclyl. In some embodiments, R^{3a} is optionally substituted 3- to 14-membered heterocyclyl comprising one or more O and/or S atoms but no N atoms. In some embodiments, R³ is optionally substituted 3- to 14-membered heterocyclyl comprising one or more N atoms and optionally one or more 0 and/S atoms. In some embodiments, R^{3a} is optionally substituted monocyclic 3- to 7-membered heterocyclyl comprising one or more O and/or S atoms but no N atoms. In some embodiments, R^{3a} is optionally substituted monocyclic 3- to 7-membered heterocyclyl comprising one or more N atoms and optionally one or more O and/or S atoms. In some embodiments, R^{3a} is saturated heterocyclyl. In some embodiments, R^{3a} is heterocyclyl comprising only one unsaturated bond (e.g., C═C bond) in the heterocyclic ring system.

In some embodiments, R^{3a} is optionally substituted monocyclic aryl. In some embodiments, R^{3a} is optionally substituted bicyclic aryl. In some embodiments, R^{3a} is optionally substituted C₆₋₁₄ aryl. In some embodiments, R^{3a} is optionally substituted C₆₋₁₀ aryl. In some embodiments, R^{3a} is optionally substituted phenyl. In some embodiments, R^{3a} is optionally substituted naphthyl.

In some embodiments, R^{3a} is optionally substituted monocyclic heteroaryl. In some embodiments, R^{3a} is optionally substituted bicyclic heteroaryl. In some embodiments, R^{3a} is optionally substituted 5- to 14-membered heteroaryl. In some embodiments, R³ is optionally substituted 5- to 10-membered heteroaryl. In some embodiments, R^{3a} is optionally substituted 5- to 6-membered monocyclic heteroaryl.

In some embodiments, R^{3a} is halogen, optionally substituted alkyl, optionally substituted alkenyl, optionally substituted carbocyclyl, or —CN. In some embodiments, R^{3a} is halogen; unsubstituted C₁₋₆ alkyl; C₁₋₄ alkyl substituted with one or more halogen and/or one or more unsubstituted, monocyclic, 3- to 7-membered carbocyclyl, as valency permits; unsubstituted C₂₋₆ alkenyl; unsubstituted, monocyclic, 3- to 7-membered carbocyclyl; or —CN. In some embodiments, R^{3a} is fluorine, chlorine, bromine, —CH₃, —C₂H₅, —CH(CH₃)₂, —CH₂C(CH₃)₃, —CF₃, —CH₂CF₃, —CH₂-(unsubstituted cyclopropyl), —CH═CH₂, unsubstituted cyclopropyl, unsubstituted cyclobutyl, or —CN. In some embodiments, R^{3a} is hydrogen; fluorine; chlorine; bromine; C₁₋₄ alkyl substituted with one or more fluorine, chlorine, and/or bromine; or —CN. In some embodiments, R^{3a} is fluorine; chlorine; bromine; C₁₋₄ alkyl substituted with one or more fluorine, chlorine, and/or bromine; or —CN.

In some embodiments, R^{3a} is not unsubstituted alkyl. In some embodiments, R^{3a} is not unsubstituted ≥C₅ alkyl.

In some embodiments, R^{3b} is halogen, optionally substituted alkyl, optionally substituted alkenyl, optionally substituted alkynyl, optionally substituted heteroalkyl, optionally substituted heteroalkenyl, optionally substituted heteroalkynyl, —OR^{A}, —SR^{A}, or —N(R^{A})₂. In some embodiments, R³⁶ is optionally substituted C₁₋₁₀ alkyl, optionally substituted C₂₋₁₀ alkenyl, optionally substituted C₂₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R^{3b} is C₁₋₁₀ haloalkyl. In some embodiments, R^{3b} is C₁₋₄ haloalkyl. In some embodiments, R^{3b} is C₁₋₄ fluoroalkyl (e.g., C₁₋₄ perfluoroalkyl). In some embodiments, R^{3b} is —CH₂F, —CHF₂, or —CF₃. In some embodiments, R^{3b} is —CF₃. In some embodiments, R^{3b} is —CH₂CH₂F, —CH₂CHF₂, or —CH₂CF₃. In some embodiments, R^{3b} is optionally substituted alkyl substituted with optionally substituted carbocyclyl. In some embodiments, R^{3b} is unsubstituted C₁₋₄ alkyl substituted with one optionally substituted C₃₋₇ monocyclic carbocyclyl. In some embodiments, R^{3b} is —CH₂-(unsubstituted cyclopropyl). In some embodiments, R^{3b} is hydrogen, optionally substituted C₁-C₆ alkyl, or halogen. In some embodiments, R^{3b} is hydrogen, fluorine, —CH₃, —CH₂F, —CHF₂, or —CF₃. In some embodiments, R³⁶ is hydrogen, fluorine, —CH₃, or —CF₃. In some embodiments, R^{3b} is hydrogen or halogen. In some embodiments, R^{3b} is hydrogen or fluorine. In some embodiments, R^{3b} is hydrogen or optionally substituted C₁-C₆ alkyl. In some embodiments, R^{3b} is hydrogen, unsubstituted C₁-C₆ alkyl, or C₁₋₆ haloalkyl. In some embodiments, R^{3b} is —CH₃. In some embodiments, R^{3b} is —C₂H₅. In some embodiments, R^{3b} is —C₃H₇, —C₄H₉, or —C₅H₁₁. In some embodiments, R^{3b} is hydrogen. In some embodiments, R^{3b} is optionally substituted alkenyl (e.g., optionally substituted C₂₋₆ alkenyl). In some embodiments, R^{3b} is unsubstituted vinyl.

In some embodiments, R^{A} is halogen. In some embodiments, R^{3b} is bromine, chlorine, or fluorine. In some embodiments, R^{3b} is bromine or chlorine. In some embodiments, R^{3b} is chlorine or fluorine. In some embodiments, R^{3b} is bromine. In some embodiments, R^{3b} is chlorine. In some embodiments, R^{3b} is fluorine.

In some embodiments, R^{3b} is —OR^{A}, —SR^{A}, or —N(R^{A})₂ (e.g., wherein R^{A} is hydrogen or optionally substituted alkyl, or optionally substituted phenyl). In some embodiments, R^{3b} is —OH. In some embodiments, R^{3b} is —SH. In some embodiments, R^{3b} is —NH₂. In some embodiments, R^{3b} is —CN, —SCN, —SSR^{A}, —N₃, —NO, or —NO₂. In some embodiments, R^{3b} is —CN. In some embodiments, R^{3b} is —C(═O)R^{A}, —C(═O)OR^{A}, —C(═O)SR^{A}, or —C(═O)N(R^{A})₂ (e.g., wherein R^{A} is hydrogen or optionally substituted alkyl, or optionally substituted phenyl). In some embodiments, R^{3b} is —C(═NR^{A})R^{A}, —C(═NR^{A})OR^{A}, —C(═NR^{A})SR^{A}, or —C(═NR^{A})N(R^{A})₂ (e.g., wherein R^{A} is hydrogen or optionally substituted alkyl, or optionally substituted phenyl). In some embodiments, R^{3b} is —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, or —S(═O)₂N(R^{A})₂ (e.g., wherein R^{A} is hydrogen or optionally substituted alkyl, or optionally substituted phenyl). In some embodiments, R^{3b} is —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)N(R^{A})₂, or —ON(R^{A})₂ (e.g., wherein R^{A} is hydrogen or optionally substituted alkyl, or optionally substituted phenyl). In some embodiments, R^{3b} is —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}. —SC(═NR^{A})SR^{A}, or —SC(═NR^{A})N(R^{A})₂ (e.g., wherein R^{A} is hydrogen or optionally substituted alkyl, or optionally substituted phenyl). In some embodiments, R^{3b} is —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}. —NR^{A}S(═O)N(R^{A})₂, —NR^{A}S(═O)₂R^{A}, —NR^{A}S(═O)₂OR^{A}, —NR^{A}S(═O)₂SR^{A}, or —NR^{A}S(═O)₂N(R^{A})₂ (e.g., wherein R^{A} is hydrogen or optionally substituted alkyl, or optionally substituted phenyl). In some embodiments, R^{3b} is —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃ (e.g., wherein R^{A} is hydrogen or optionally substituted alkyl, or optionally substituted phenyl).

In some embodiments, R^{3b} is optionally substituted carbocyclyl, optionally substituted heterocyclyl, optionally substituted aryl, or optionally substituted heteroaryl. In some embodiments, R^{3b} is optionally substituted C₃₋₁₄ carbocyclyl. In some embodiments, R^{3b} is optionally substituted monocyclic C₃₋₇ carbocyclyl. In some embodiments, R^{3b} is optionally substituted monocyclic C₃₋₄ carbocyclyl. In some embodiments, R^{3b} is optionally substituted cyclopropyl. In some embodiments, R^{3b} is optionally substituted cyclobutyl. In some embodiments, R^{3b} is optionally substituted monocyclic C₅₋₇ carbocyclyl. In some embodiments, R^{3b} is saturated carbocyclyl. In some embodiments, R^{3b} is carbocyclyl comprising only one unsaturated bond (e.g., C═C bond) in the carbocyclic ring system.

In some embodiments, R^{3b} is optionally substituted 3- to 14-membered heterocyclyl. In some embodiments, R^{3b} is optionally substituted monocyclic 3- to 7-membered heterocyclyl. In some embodiments, R^{3b} is optionally substituted 3- to 14-membered heterocyclyl comprising one or more O and/or S atoms but no N atoms. In some embodiments, R^{3b} is optionally substituted 3- to 14-membered heterocyclyl comprising one or more N atoms and optionally one or more O and/S atoms. In some embodiments, R^{3b} is optionally substituted monocyclic 3- to 7-membered heterocyclyl comprising one or more O and/or S atoms but no N atoms. In some embodiments, R^{3b} is optionally substituted monocyclic 3- to 7-membered heterocyclyl comprising one or more N atoms and optionally one or more O and/or S atoms. In some embodiments, R^{3b} is saturated heterocyclyl. In some embodiments, R^{3b} is heterocyclyl comprising only one unsaturated bond (e.g., C═C bond) in the heterocyclic ring system.

In some embodiments, R^{3b} is optionally substituted monocyclic aryl. In some embodiments, R³ is optionally substituted bicyclic aryl. In some embodiments, R^{3b} is optionally substituted C₁₋₄ aryl. In some embodiments, R^{3b} is optionally substituted C₆₋₁₀ aryl. In some embodiments, R^{3b} is optionally substituted phenyl. In some embodiments, R^{3b} is optionally substituted naphthyl.

In some embodiments, R^{3b} is optionally substituted monocyclic heteroaryl. In some embodiments, R^{3b} is optionally substituted bicyclic heteroaryl. In some embodiments, R^{3b} is optionally substituted 5- to 14-membered heteroaryl. In some embodiments, R^{3b} is optionally substituted 5- to 10-membered heteroaryl. In some embodiments, R^{3b} is optionally substituted 5- to 6-membered monocyclic heteroaryl.

In certain embodiments, R^{3b} is halogen, optionally substituted alkyl, optionally substituted carbocyclyl, or —CN. In certain embodiments, R^{3b} is halogen; unsubstituted C₁₋₆ alkyl; C₁₋₆ alkyl substituted with one or more halogen, as valency permits; unsubstituted, monocyclic, 3- to 7-membered carbocyclyl; or —CN. In certain embodiments, R^{3b} is fluorine, chlorine, bromine, —CH₃, —C₂H₅, —CH(CH₃)₂, —CH₂C(CH₃)₃, —CF₃, —CH₂CF₃, —CH₂-(unsubstituted cyclopropyl), —CH═CH₂, unsubstituted cyclopropyl, unsubstituted cyclobutyl, or —CN. In certain embodiments, R^{3b} is fluorine or —CF₃. In some embodiments, R^{3b} is hydrogen; fluorine; chlorine; bromine; C₁₋₄ alkyl substituted with one or more fluorine, chlorine, and/or bromine; or —CN. In some embodiments, R^{3b} is fluorine; chlorine; bromine; C₁₋₄ alkyl substituted with one or more fluorine, chlorine, and/or bromine; or —CN.

In some embodiments, R^{3b} is not unsubstituted alkyl. In some embodiments, R^{3b} is not unsubstituted ≥C₅ alkyl.

In some embodiments, each of R³, and R^{3b} is independently hydrogen; fluorine; chlorine; bromine; C₁₋₄ alkyl substituted with one or more fluorine, chlorine, and/or bromine; or —CN.

In some embodiments, each of R^{3a} and R^{3b} is independently hydrogen, fluorine, or C₁₋₂ alkyl substituted with one or more fluorine.

In some embodiments, at least one of R^{3a} and R^{3b} is fluorine; chlorine; bromine; C₁₋₄ alkyl substituted with one or more fluorine, chlorine, and/or bromine; or —CN. In some embodiments, at least one of R^{3a} and R^{3b} is fluorine or C₁₋₂ alkyl substituted with one or more fluorine.

In certain embodiments, R^{3a} and R^{3b} are taken together with their intervening atoms to form an optionally substituted, monocyclic, carbocyclic or heterocyclic ring. In certain embodiments, R^{3a} and R^{3b} are taken together with their intervening atoms to form an optionally substituted, monocyclic, 3- to 7-membered carbocyclic ring. In certain embodiments, R^{3a} and R^{3b} are taken together with their intervening atoms to form an optionally substituted, monocyclic, 5-membered carbocyclic ring.

In certain embodiments, R^{3a} and R^{3b} are taken together with their intervening atoms to form an optionally substituted, monocyclic, 3- to 7-membered heterocyclic ring. In some embodiments, R^{3a} and R^{3b} are taken together with their intervening atoms to form an optionally substituted, 5-membered, monocyclic, heterocyclic ring. In some embodiments, R^{3a} and R^{3b} are taken together with their intervening atoms to form an optionally substituted, 6-membered, monocyclic, heterocyclic ring. In some embodiments, R^{3a} and R^{3b} are taken together with their intervening atoms to form an optionally substituted, monocyclic, heterocyclic ring comprising one or more oxygen atoms. In some embodiments, R^{3a} and R^{3b} are taken together with their intervening atoms to form an optionally substituted, monocyclic, heterocyclic ring comprising one or more nitrogen atoms. In some embodiments, R^{3a} and R^{3b} are taken together with their intervening atoms to form an optionally substituted, monocyclic, heterocyclic ring comprising no nitrogen atoms.

In certain embodiments, R^{3a} and R^{3b} are taken together with their intervening atoms to form an optionally substituted phenyl ring.

In certain embodiments, R^{3a} and R^{3b} are taken together with their intervening atoms to form an optionally substituted, monocyclic, heteroaryl ring. In certain embodiments, R^{3a} and R^{3b} are taken together with their intervening atoms to form an optionally substituted, 5-membered, monocyclic, heteroaryl ring. In certain embodiments, R^{3a} and R^{3b} are taken together with their intervening atoms to form an optionally substituted, 6-membered, monocyclic, heteroaryl ring. In some embodiments, R^{3a} and R^{3b} are taken together with their intervening atoms to form an optionally substituted, monocyclic, heteroaryl ring comprising one or more oxygen and/or sulfur atoms. In some embodiments, R^{3a} and R^{3b} are taken together with their intervening atoms to form an optionally substituted, monocyclic, heteroaryl ring comprising one or more nitrogen atoms. In some embodiments, R^{3a} and R^{3b} are taken together with their intervening atoms to form an optionally substituted, monocyclic, heteroaryl ring comprising no nitrogen atoms.

In some embodiments, R^{3a} and R^{3b} are not taken together with their intervening atoms to form an optionally substituted, monocyclic, carbocyclic, heterocyclic, or heteroaryl ring, or an optionally substituted phenyl ring. In some embodiments, each of R^{3a} and R^{3b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR⁴C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃.

In some embodiments, at least one instance of R^{A} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{A} is optionally substituted carbocyclyl, optionally substituted heterocyclyl, optionally substituted aryl, or optionally substituted heteroaryl. In some embodiments, at least one instance of R^{A} is independently hydrogen, optionally substituted C₁₋₁₀ alkyl, optionally substituted C₂₋₁₀ alkenyl, optionally substituted C₂₋₁₀ alkynyl, optionally substituted C₃₋₁₄ carbocyclyl, or optionally substituted C₆₋₁₄ aryl. In some embodiments, at least one instance of R^{A} is independently hydrogen, optionally substituted C₁₋₁₀ alkyl, or optionally substituted phenyl. In some embodiments, at least one instance of R^{A} is a nitrogen protecting group when attached to a nitrogen atom, an oxygen protecting group when attached to an oxygen atom, or a sulfur protecting group when attached to a sulfur atom. In some embodiments, two instances of R^{A} attached to the same intervening atom are joined together with the intervening atom to form an optionally substituted, monocyclic, heterocyclic or heteroaryl ring.

In some embodiments, at least one of R^{4a} and R^{4b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, at least one of R⁴ and R^{4b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, at least one of R^{4a} and R^{4b} is independently hydrogen, halogen, or optionally substituted alkyl. In some embodiments, each of R^{4a} and R^{4b} is independently hydrogen, halogen, or optionally substituted alkyl. In some embodiments, at least one of R^{4a} and R^{4b} is independently hydrogen, halogen, or optionally substituted C₁₋₄ alkyl. In some embodiments, at least one of R^{4a} and R^{4b} is independently hydrogen, fluorine, or optionally substituted C₁₋₄ alkyl. In some embodiments, at least one of R^{4a} and R^{4b} is independently hydrogen or halogen. In some embodiments, at least one of R^{4a} and R^{4b} is independently hydrogen or fluorine. In some embodiments, at least one of R^{4a} and R^{4b} is independently hydrogen or optionally substituted C₁₋₄ alkyl. In some embodiments, at least one of R^{4a} and R^{4b} is hydrogen. In some embodiments, at least one of R^{4a} and R^{4b} is hydrogen. In some embodiments, at least one of R^{4a} and R^{4b} is optionally substituted C₁₋₄ alkyl. In some embodiments, at least one of R^{4a} and R^{4b} is unsubstituted methyl. In some embodiments, at least one of R^{4a} and R^{4b} is optionally substituted C₁₋₄ alkyl. In some embodiments, at least one of R^{4a} and R^{4b} is unsubstituted methyl. In some embodiments, at least one of R^{4a} and R^{4b} is fluorine.

In some embodiments, R^{4a} is hydrogen. In some embodiments, R^{4b} is hydrogen. In some embodiments, R^{4b} is halogen. In some embodiments, R^{b} is fluorine. In some embodiments, each of R^{4a} and R^{4b} is hydrogen. In some embodiments, R^{4a} is hydrogen, and R^{4b} is fluorine. In some embodiments, R^{4a} is fluorine, and R^{4b} is hydrogen.

In some embodiments, R^{4s} and R^{4b} are taken together with their intervening atom to form an optionally substituted, monocyclic, carbocyclic or heterocyclic ring. In some embodiments, R^{4a} and R^{4b} are taken together with their intervening atom to form an optionally substituted, monocyclic, C₃₋₁₄ carbocyclic ring. In some embodiments, R^{4a} and R^{4b} are taken together with their intervening atom to form an optionally substituted, monocyclic, C₃₋₇ carbocyclic ring. In certain embodiments, R^{4a} and R^{4b} are taken together with their intervening atom to form an optionally substituted, cyclopropyl, cyclobutyl, cyclopentyl, or cyclohexyl ring. In some embodiments, R^{4s} and R^{4b} are taken together with their intervening atom to form an optionally substituted, monocyclic, 3- to 14-membered heterocyclic ring. In some embodiments, R^{4A} and R^{4B} are taken together with their intervening atom to form an optionally substituted, monocyclic, 3- to 7-membered heterocyclic ring. In some embodiments, R^{4a} and R^{4b} are taken together with their intervening atom to form an optionally substituted, monocyclic, heterocyclic ring comprising O and/or S heteroatom(s) as the only heteroatoms in the heterocyclic ring. In some embodiments, R^{4a} and R^{4b} are taken together with their intervening atom to form an optionally substituted, monocyclic, heterocyclic ring comprising O and/or N heteroatom(s) as the only heteroatoms in the heterocyclic ring. In some embodiments, R^{4a} and R^{4b} are taken together with their intervening atom to form optionally substituted oxetane, optionally substituted tetrahydrofuran, optionally substituted tetrahydropyran, or optionally substituted pyrrolidine.

In certain embodiments,

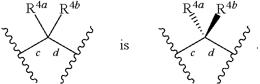

In certain embodiments,

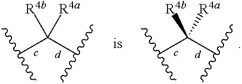

In some embodiments, R^{4b} and R^{5a} are taken together with their intervening atom to form an optionally substituted, monocyclic, carbocyclic, heterocyclic, aryl, or heteroaryl ring. In some embodiments, R^{4b} and R^{5a} are taken together with their intervening atom to form an optionally substituted, monocyclic, carbocyclic or heterocyclic ring. In some embodiments, R^{4b} and R^{5a} are taken together with their intervening atom to form an optionally substituted, monocyclic, C₃₋₁₄ carbocyclic ring. In some embodiments, R^{4b} and RSS are taken together with their intervening atom to form an optionally substituted, monocyclic, C₃₋₇ carbocyclic ring. In some embodiments, R^{4b} and R^{5a} are taken together with their intervening atom to form an optionally substituted, monocyclic, 3- to 14-membered heterocyclic ring. In some embodiments, R^{4b} and R^{5a} are taken together with their intervening atom to form an optionally substituted, monocyclic, 3- to 7-membered heterocyclic ring. In some embodiments, R^{4b} and R^{5a} are taken together with their intervening atom to form an optionally substituted, monocyclic, aryl, or heteroaryl ring. In some embodiments, R^{4b} and R^{5a} are taken together with their intervening atom to form an optionally substituted phenyl ring. In some embodiments, Re and R⁵ are taken together with their intervening atom to form an optionally substituted, monocyclic, 5- or 6-membered heteroaryl ring.

In some embodiments, at least one of R^{5a} and R^{5b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, at least one of R^{5a} and R^{5b} is independently hydrogen, halogen, or optionally substituted alkyl. In some embodiments, each of R^{5a} and R^{5b} is independently hydrogen, halogen, or optionally substituted alkyl. In some embodiments, at least one of R^{5a} and R^{5b} is independently hydrogen, —CH₃, or —CF₃. In some embodiments, at least one of R^{5a} and R^{5b} is hydrogen. In some embodiments, at least one of R^{5a} and R^{5b} is independently optionally substituted C₁₋₁₀ alkyl, optionally substituted C₂₋₁₀ alkenyl, or optionally substituted C₂₋₁₀ alkynyl. In some embodiments, at least one of R⁵ and R⁵¹ is independently optionally substituted C₁₋₁₀ heteroalkyl, optionally substituted C₁₋₁₀ heteroalkenyl, or optionally substituted C₁₋₁₀ heteroalkynyl. In some embodiments, at least one of R^{5a} and R^{5b} is independently optionally substituted C₁₋₁₀ alkyl or optionally substituted C₁₋₁₀ heteroalkyl. In some embodiments, at least one of R^{5a} and R^{5b} is optionally substituted C₁₋₄ alkyl. In some embodiments, at least one of R^{5a} and R^{5b} is —CH₃. In some embodiments, at least one of R^{5a} and R^{5b} is —CF₃. In some embodiments, at least one of R^{5a} and R^{5b} is optionally substituted C₁₋₄ heteroalkyl. In some embodiments, at least one of R^{5a} and R^{5b} is optionally substituted C₁₋₄ heteroalkyl comprising an O atom. In some embodiments, at least one of R^{5a} and R^{5b} is optionally substituted C₆₋₁₄ aryl. In some embodiments, at least one of R^{5a} and R^{5b} is optionally substituted C₆₋₁₀ aryl. In some embodiments, at least one of R^{5a} and R^{5b} is optionally substituted phenyl. In some embodiments, R^{5a} and R^{5b} are —CH₃. In some embodiments, R^{5a} and R^{5b} are hydrogen.

In some embodiments, R^{5a} and R^{5b} are taken together with their intervening atom to form an optionally substituted, monocyclic, carbocyclic or heterocyclic ring. In some embodiments. R^{5a} and R^{5b} are taken together with their intervening atom to form an optionally substituted, monocyclic, carbocyclic ring. In some embodiments, R^{5a} and R^{5b} are taken together with their intervening atom to form an optionally substituted, monocyclic, C₃₋₁₄ carbocyclic ring. In some embodiments, R^{5a} and R^{5b} are taken together with their intervening atom to form an optionally substituted, monocyclic, C₃₋₇ carbocyclic ring. In some embodiments, R^{5a} and R^{5b} are taken together with their intervening atom to form an optionally substituted cyclopropyl, cyclobutyl, cyclopentyl, or cyclohexyl ring. In some embodiments, R^{5a} and R^{5b} are taken together with their intervening atom to form an optionally substituted cyclobutyl ring. In some embodiments, R^{5a} and R^{5b} are taken together with their intervening atom to form an unsubstituted, monocyclic, 3- to 7-membered carbocyclic ring. In some embodiments, RSS and R^{5b} are taken together with their intervening atom to form an optionally substituted, monocyclic, heterocyclic ring. In some embodiments, R^{5a} and R^{5b} are taken together with their intervening atom to form an optionally substituted, monocyclic, heterocyclic ring comprising O and/or S heteroatom(s) as the only heteroatoms in the heterocyclic ring. In some embodiments, R^{5a} and R^{5b} are taken together with their intervening atom to form an optionally substituted, monocyclic, heterocyclic ring comprising O and/or N heteroatom(s) as the only heteroatoms in the heterocyclic ring. In some embodiments, R^{5a} and R^{5b} are taken together with their intervening atom to form an optionally substituted, monocyclic, 3- to 14-membered heterocyclic ring. In some embodiments, R^{5a} and R^{5b} are taken together with their intervening atom to form an optionally substituted, monocyclic, 3- to 7-membered heterocyclic ring. In some embodiments, R^{5a} and R^{5b} are taken together with their intervening atom to form an optionally substituted, monocyclic, 5- to 6-membered heterocyclic ring. In some embodiments, R^{5a} and R^{5b} are taken together with their intervening atom to form optionally substituted oxetane, optionally substituted tetrahydrofuran, optionally substituted tetrahydropyran, or optionally substituted pyrrolidine.

In some embodiments, R^{5a} is optionally substituted alkyl. In some embodiments, R^{5a} is unsubstituted C₁₋₄ alkyl. In some embodiments, R^{5a} is —CH₃. In some embodiments, R^{5b} is optionally substituted alkyl. In some embodiments, R^{5b} is unsubstituted C₁₋₄ alkyl. In some embodiments, R^{5b} is —CH₃.

In certain embodiments,

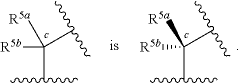

In certain embodiments,

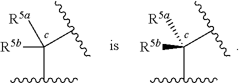

In certain embodiments, R^{3c} is protium. In certain embodiments, R^{3c} is deuterium.

In some embodiments, X is

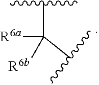

In certain embodiments,

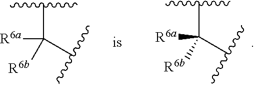

In certain embodiments,

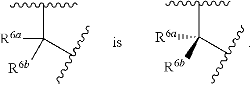

In some embodiments, X is —N(R⁶)—. In some embodiments, X is —NH—.

In some embodiments, R⁶ is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R⁶ is hydrogen, optionally substituted alkyl, or a nitrogen protecting group. In some embodiments, R⁶ is hydrogen. In some embodiments, R⁶ is optionally substituted C₁₋₁₀ alkyl, optionally substituted C₂₋₁₀ alkenyl, or C₂₋₁₀ alkynyl. In some embodiments, R⁶ is optionally substituted alkyl. In some embodiments, R⁶ is optionally substituted C₁₋₁₀ alkyl. In some embodiments, R⁶ is optionally substituted C₁₋₆ alkyl. In some embodiments, R⁶ is optionally substituted C₁₋₄ alkyl. In some embodiments, R⁶ is unsubstituted methyl. In some embodiments, R⁶ is benzyl.

In some embodiments, R⁶ is optionally substituted carbocyclyl or optionally substituted heterocyclyl. In some embodiments, R⁶ is optionally substituted C₃₋₁₄ carbocyclyl. In some embodiments, R⁶ is optionally substituted monocyclic C₃₋₇ carbocyclyl. In some embodiments. R⁶ is optionally substituted monocyclic C₅₋₇ carbocyclyl. In some embodiments, R⁶ is saturated carbocyclyl. In some embodiments, R⁶ is carbocyclyl comprising only one unsaturated bond (e.g., C═C bond) in the carbocyclic ring system.

In some embodiments, R⁶ is optionally substituted 3- to 14-membered heterocyclyl. In some embodiments, R⁶ is optionally substituted monocyclic 3- to 7-membered heterocyclyl. In some embodiments, R⁶ is optionally substituted heterocyclyl comprising one or more O and/or S atoms, but no N atoms. In some embodiments, R⁶ is optionally substituted 3- to 14-membered heterocyclyl comprising one or more N atoms and optionally one or more O and/S atoms. In some embodiments, R⁶ is optionally substituted monocyclic 3- to 7-membered heterocyclyl comprising one or more O and/or S atoms but no N atoms. In some embodiments, R⁶ is optionally substituted monocyclic 3- to 7-membered heterocyclyl comprising one or more N atoms and optionally one or more O and/or S atoms. In some embodiments, R⁶ is saturated heterocyclyl. In some embodiments, R⁶ is heterocyclyl comprising only one unsaturated bond (e.g., C═C bond) in the heterocyclic ring system.

In some embodiments, R⁶ is optionally substituted aryl or optionally substituted heteroaryl. In some embodiments, R⁶ is optionally substituted monocyclic aryl. In some embodiments, R⁶ is optionally substituted bicyclic aryl. In some embodiments, R⁶ is optionally substituted C₆₋₁₄ aryl. In some embodiments, R⁶ is optionally substituted C₆₋₁₀ aryl. In some embodiments, R⁶ is optionally substituted phenyl. In some embodiments, R⁶ is optionally substituted naphthyl.

In some embodiments, R⁶ is optionally substituted monocyclic heteroaryl. In some embodiments, R⁶ is optionally substituted bicyclic heteroaryl. In some embodiments, R⁶ is optionally substituted 5- to 14-membered heteroaryl. In some embodiments, R⁶ is optionally substituted 5- to 10-membered heteroaryl. In some embodiments, R⁶ is optionally substituted 5- to 6-membered monocyclic heteroaryl. In some embodiments, R⁶ is optionally substituted heteroaryl comprising one or more N atoms. In some embodiments, R⁶ is optionally substituted pyridyl, optionally substituted phenyl, optionally substituted pyrimidinyl, optionally substituted imidazolyl, optionally substituted pyrazolyl, or optionally substituted isothiazolyl. In some embodiments, R⁶ is optionally substituted pyridyl, optionally substituted phenyl, or optionally substituted pyrimidinyl.

In some embodiments, R⁶ is a nitrogen protecting group (e.g., Bn, Boc, Cbz, Fmoc, trifluoroacetyl, triphenylmethyl, acetyl, or Ts).

In some embodiments, R^{6a} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R^{6a} is hydrogen. In some embodiments, R^{6a} is optionally substituted C₁₋₁₀ alkyl, optionally substituted C₂₋₁₀ alkenyl, or C₂₋₁₀ alkynyl. In some embodiments, R^{6a} is optionally substituted alkyl. In some embodiments, R^{6s} is optionally substituted C₁₋₁₀ alkyl. In some embodiments, R^{6s} is optionally substituted C₁₋₆ alkyl. In some embodiments, R^{6a} is optionally substituted C₁₋₄ alkyl. In some embodiments, R^{6a} is unsubstituted methyl. In some embodiments, R^{6a} is benzyl.

In some embodiments, R^{6a} is optionally substituted carbocyclyl or optionally substituted heterocyclyl. In some embodiments, R^{6a} is optionally substituted C₃₋₁₄ carbocyclyl. In some embodiments, R^{6a} is optionally substituted monocyclic C₃₋₇ carbocyclyl. In some embodiments, R^{6a} is optionally substituted monocyclic C₅₋₇ carbocyclyl. In some embodiments, R^{6a} is saturated carbocyclyl. In some embodiments, R^{6a} is carbocyclyl comprising only one unsaturated bond (e.g., C═C bond) in the carbocyclic ring system.

In some embodiments, R^{6a} is optionally substituted 3- to 14-membered heterocyclyl. In some embodiments, R⁶¹ is optionally substituted monocyclic 3- to 7-membered heterocyclyl. In some embodiments, R^{6a} is optionally substituted heterocyclyl comprising one or more O and/or S atoms, but no N atoms. In some embodiments, R^{6a} is optionally substituted 3- to 14-membered heterocyclyl comprising one or more N atoms and optionally one or more O and/S atoms. In some embodiments, R^{6a} is optionally substituted monocyclic 3- to 7-membered heterocyclyl comprising one or more O and/or S atoms but no N atoms. In some embodiments, R^{6a} is optionally substituted monocyclic 3- to 7-membered heterocyclyl comprising one or more N atoms and optionally one or more O and/or S atoms. In some embodiments, R^{6a} is saturated heterocyclyl. In some embodiments, R^{6a} is heterocyclyl comprising only one unsaturated bond (e.g., C═C bond) in the heterocyclic ring system.

In some embodiments, R^{6a} is optionally substituted aryl or optionally substituted heteroaryl. In some embodiments, R^{6a} is optionally substituted monocyclic aryl. In some embodiments, R^{6a} is optionally substituted bicyclic aryl. In some embodiments, R^{6a} is optionally substituted C₆₋₁₄ aryl. In some embodiments, R^{6a} is optionally substituted C₆₋₁₀ aryl. In some embodiments, R^{6a} is optionally substituted phenyl. In some embodiments, R^{6a} is optionally substituted naphthyl.

In some embodiments, R^{6a} is optionally substituted monocyclic heteroaryl. In some embodiments, R^{6a} is optionally substituted bicyclic heteroaryl. In some embodiments, R^{6a} is optionally substituted 5- to 14-membered heteroaryl. In some embodiments, R^{6a} is optionally substituted 5- to 10-membered heteroaryl. In some embodiments, R^{6a} is optionally substituted 5- to 6-membered monocyclic heteroaryl. In some embodiments, R^{6a} is optionally substituted heteroaryl comprising one or more N atoms. In some embodiments, R^{6a} is optionally substituted pyridyl, optionally substituted phenyl, optionally substituted pyrimidinyl, optionally substituted imidazolyl, optionally substituted pyrazolyl, or optionally substituted isothiazolyl. In some embodiments, R^{6a} is optionally substituted pyridyl, optionally substituted phenyl, or optionally substituted pyrimidinyl.

In some embodiments, R^{6b} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R^{6b} is hydrogen. In some embodiments, R^{6b} is optionally substituted C₁₋₁₀ alkyl, optionally substituted C₂₋₁₀ alkenyl, or C₂₋₁₀ alkynyl. In some embodiments, R^{6b} is optionally substituted alkyl. In some embodiments, R^{6b} is optionally substituted C₁₋₁₀ alkyl. In some embodiments, R^{6b} is optionally substituted C₁₋₆ alkyl. In some embodiments, R^{6b} is optionally substituted C₁₋₄ alkyl. In some embodiments, R^{6b} is unsubstituted methyl. In some embodiments, R^{6b} is benzyl.

In some embodiments, R^{6b} is optionally substituted carbocyclyl or optionally substituted heterocyclyl. In some embodiments, R^{6b} is optionally substituted C₃₋₁₄ carbocyclyl. In some embodiments, R^{6b} is optionally substituted monocyclic C₃₋₇ carbocyclyl. In some embodiments. R^{6b} is optionally substituted monocyclic C₅₋₇ carbocyclyl. In some embodiments, R^{6b} is saturated carbocyclyl. In some embodiments, R^{6b} is carbocyclyl comprising only one unsaturated bond (e.g., C═C bond) in the carbocyclic ring system.

In some embodiments, R^{6b} is optionally substituted 3- to 14-membered heterocyclyl. In some embodiments, R^{6b} is optionally substituted monocyclic 3- to 7-membered heterocyclyl. In some embodiments, R^{6b} is optionally substituted heterocyclyl comprising one or more O and/or S atoms, but no N atoms. In some embodiments, R^{6b} is optionally substituted 3- to 14-membered heterocyclyl comprising one or more N atoms and optionally one or more O and/S atoms. In some embodiments, R^{6b} is optionally substituted monocyclic 3- to 7-membered heterocyclyl comprising one or more O and/or S atoms but no N atoms. In some embodiments, R^{6b} is optionally substituted monocyclic 3- to 7-membered heterocyclyl comprising one or more N atoms and optionally one or more O and/or S atoms. In some embodiments, R^{6b} is saturated heterocyclyl. In some embodiments, R^{6b} is heterocyclyl comprising only one unsaturated bond (e.g., C═C bond) in the heterocyclic ring system.

In some embodiments, R^{6b} is optionally substituted aryl or optionally substituted heteroaryl. In some embodiments, R^{6b} is optionally substituted monocyclic aryl. In some embodiments, R^{6b} is optionally substituted bicyclic aryl. In some embodiments, R^{6b} is optionally substituted C₆₋₁₄ aryl. In some embodiments, R^{6a} is optionally substituted C₆₋₁₀ aryl. In some embodiments, R^{6b} is optionally substituted phenyl. In some embodiments, R^{6a} is optionally substituted naphthyl.

In some embodiments, R^{6b} is optionally substituted monocyclic heteroaryl. In some embodiments, R^{6b} is optionally substituted bicyclic heteroaryl. In some embodiments, R^{6b} is optionally substituted 5- to 14-membered heteroaryl. In some embodiments, R^{6b} is optionally substituted 5- to 10-membered heteroaryl. In some embodiments, R^{6b} is optionally substituted 5- to 6-membered monocyclic heteroaryl. In some embodiments, R^{6b} is optionally substituted heteroaryl comprising one or more N atoms. In some embodiments, R^{6b} is optionally substituted pyridyl, optionally substituted phenyl, optionally substituted pyrimidinyl, optionally substituted imidazolyl, optionally substituted pyrazolyl, or optionally substituted isothiazolyl. In some embodiments, R^{6b} is optionally substituted pyridyl, optionally substituted phenyl, or optionally substituted pyrimidinyl.

In some embodiments, each of R^{6a} and R^{6b} is independently hydrogen, halogen, or optionally substituted alkyl.

In some embodiments, the molecular weight of the compound is not greater than 500 g/mol. In some embodiments, the molecular weight of the compound is between 200 and 300, between 300 and 400, between 400 and 500, between 500 and 600, between 600 and 700, between 700 and 800, or between 800 and 1000, inclusive, g/mol. In certain embodiments, the compound comprises 1, 2, or 3 hydrogen bond donors. In certain embodiments, the compound comprises 1, 2, or 3 hydrogen bond acceptors. In certain embodiments, the compound comprises 2, 3, 4, or 5 hydrogen bond donors and hydrogen bond acceptors as combined.

In some embodiments, the compound is of Formula I-1:

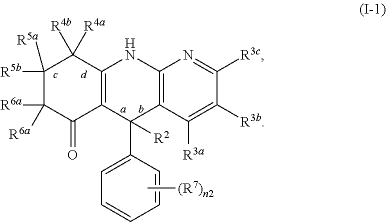

or a pharmaceutically acceptable salt, solvate, hydrate, polymorph, co-crystal, tautomer, stereoisomer, isotopically labeled compound, or prodrug thereof.

In some embodiments, the compound is of Formula I-2:

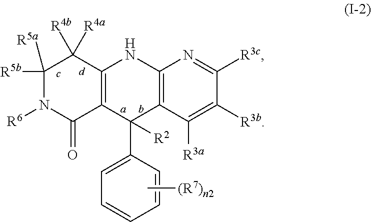

or a pharmaceutically acceptable salt, solvate, hydrate, polymorph, co-crystal, tautomer, stereoisomer, isotopically labeled compound, or prodrug thereof.

In some embodiments, the compound is of Formula I-3:

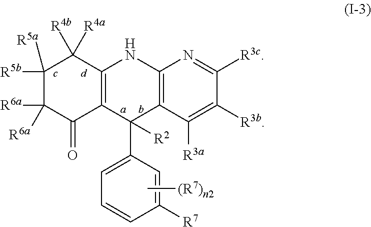

or a pharmaceutically acceptable salt, solvate, hydrate, polymorph, co-crystal, tautomer, stereoisomer, isotopically labeled compound, or prodrug thereof.

In some embodiments, the compound is of Formula I-4:

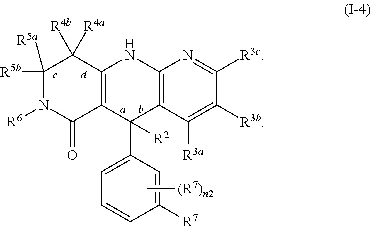

or a pharmaceutically acceptable salt, solvate, hydrate, polymorph, co-crystal, tautomer, stereoisomer, isotopically labeled compound, or prodrug thereof.

In some embodiments, the compound is of any one of the formulae shown in Table 1:

TABLE 1

| No. | Formula |
|---|---|
| 101R | 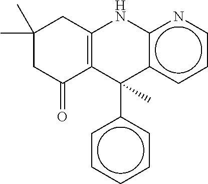 |
| 101S | 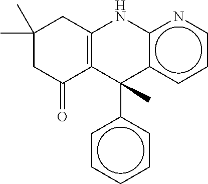 |
| 102R | 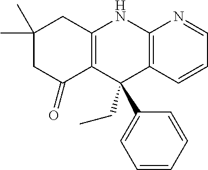 [SMILES: CC[C@]1(c2ccccc2)C2=C(CC(C)(C)CC2=O)Nc2ncccc21] |
| 102S | 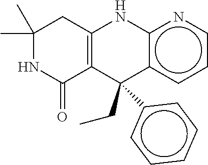 |
| 103R | 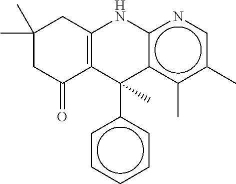 |
| 103S | 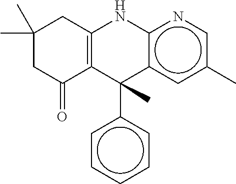 |
| 104R | 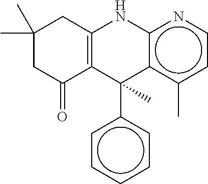 |
| 104S | 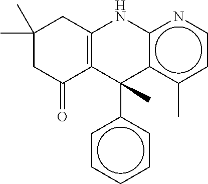 |
| 105R | 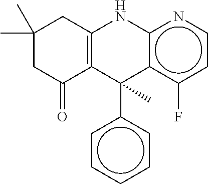 |
| 105S | 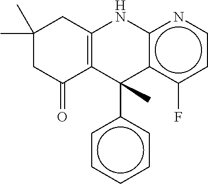 |
| 107R | 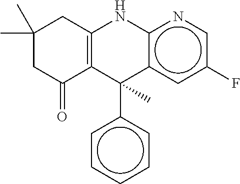 |
| 107S | 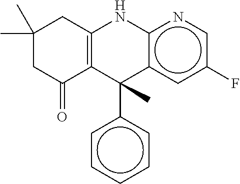 |
| 108R | 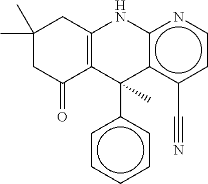 |
| 108S | 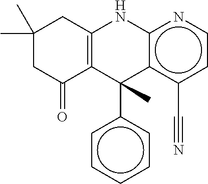 |
| 109R | 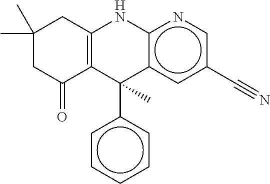 |
| 109S | 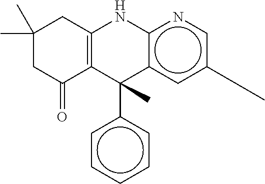 |
| 110R | 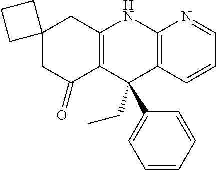 [SMILES: CC[C@]1(c2ccccc2)C2=C(CC3(CCC3)CC2=O)Nc2ncccc21] |
| 110S | 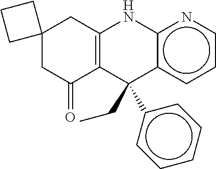 |
| 111R | 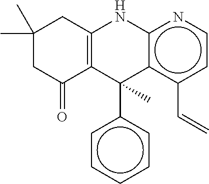 |
| 111S | 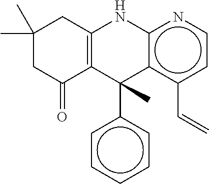 |
| 112R | 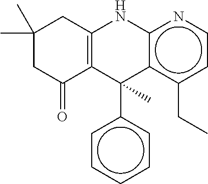 |
| 112S | 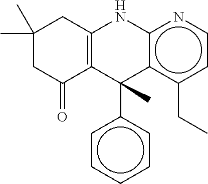 |
| 113R | 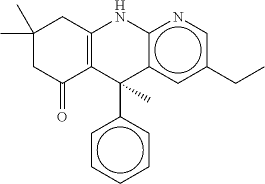 |
| 113S | 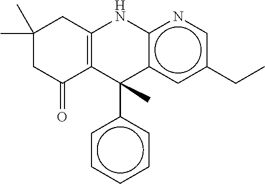 |
| 114R | 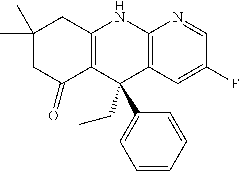 [SMILES: CC[C@]1(c2ccccc2)C2=C(CC(C)(C)CC2=O)Nc2ncc(F)cc21] |
| 114S | 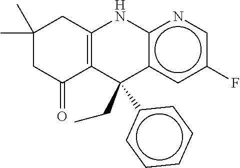 |
| 115R | 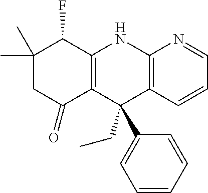 [SMILES: CC[C@]1(c2ccccc2)C2=C(Nc3ncccc31)[C@@H](F)C(C)(C)CC2=O] |
| 115S | 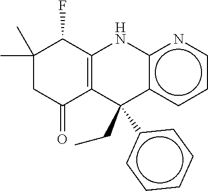 |
| 116R | 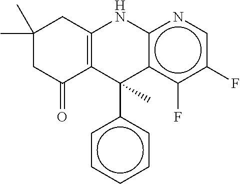 |
| 116S | 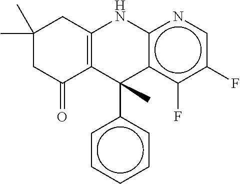 |
| 117R | 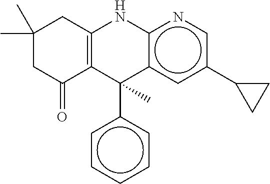 |
| 117S | 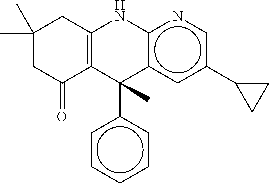 |
| 118R | 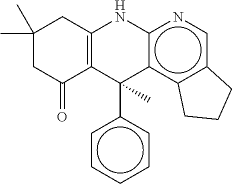 |
| 118S | 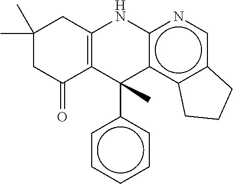 |
| 119R | 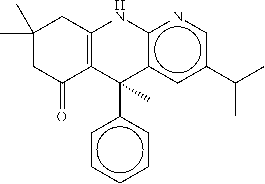 |
| 119S | 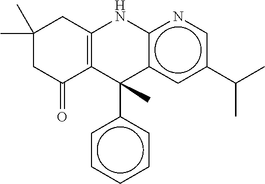 |
| 120R | 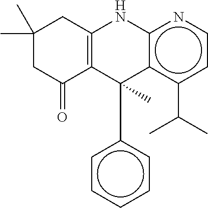 |
| 120S | 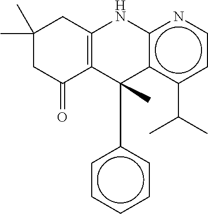 |
| 121R | 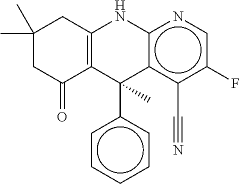 |
| 121S | 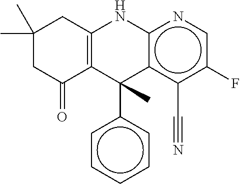 |
| 122R | 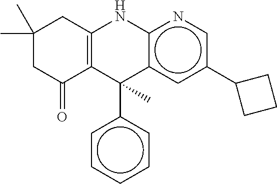 |
| 122S | 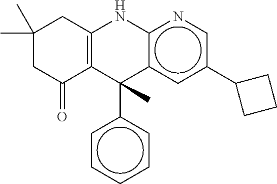 |
| 123R | 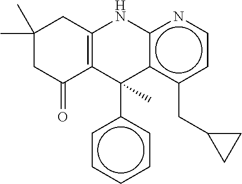 |
| 123S | 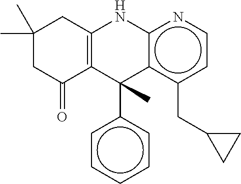 |
| 124R | 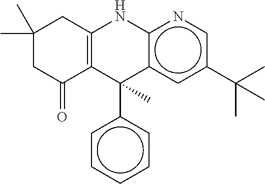 |
| 124S | 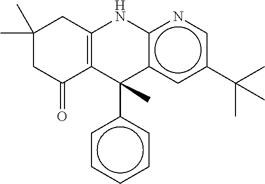 |
| 125R | 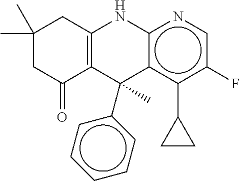 |
| 125S | 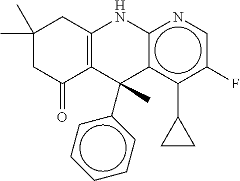 |
| 126R | 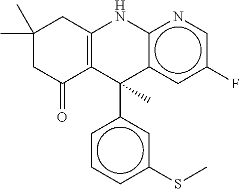 |
| 126S | 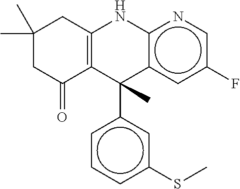 |
| 127R | 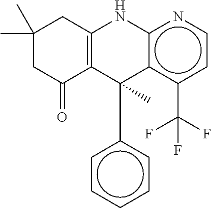 |
| 127S | 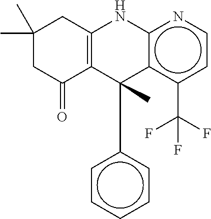 |
| 128R | 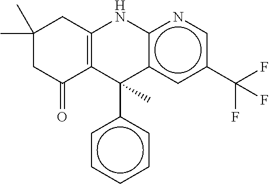 |
| 128S | 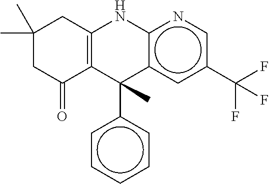 |
| 129R | 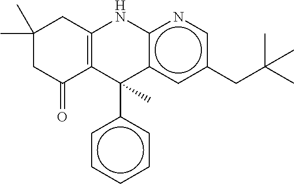 |
| 129S | 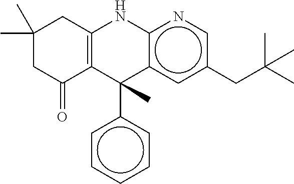 |
| 130R | 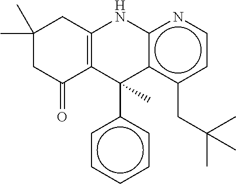 |
| 130S | 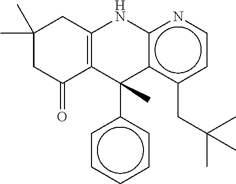 |
| 131R | 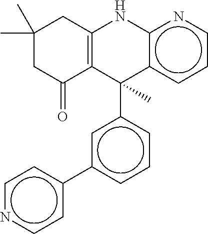 |
| 131S | 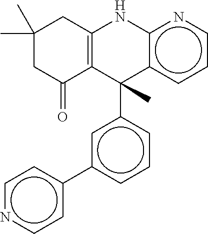 |
| 132R | 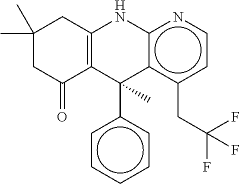 |
| 132S | 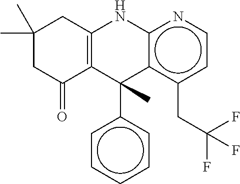 |
| 133R | 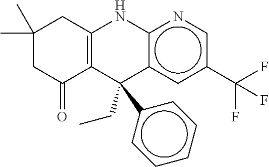 |
| 133S | 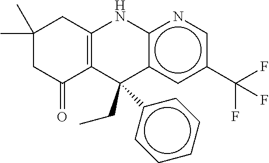 |
| 134R | 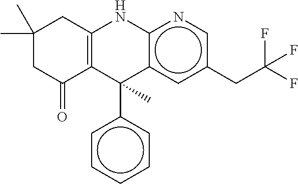 |
| 134S | 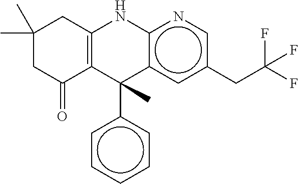 |
| 135R | 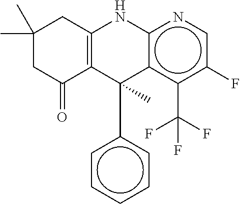 |
| 135S | 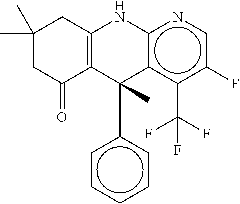 |
| 136R | 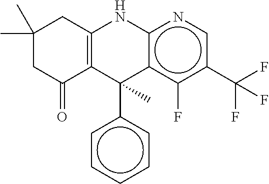 |
| 136S | 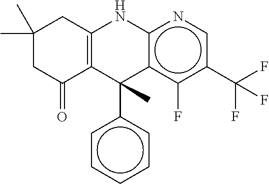 |
| 137R | 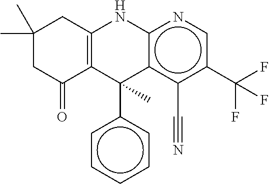 |
| 137S | 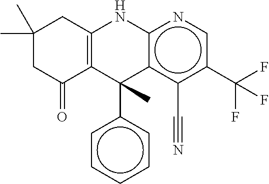 |
| 138R | 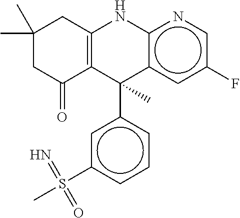 |
| 138S | 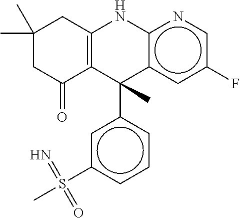 |
| 139R | 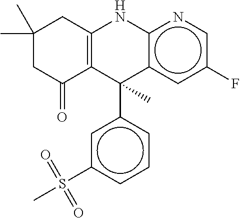 |
| 139S | 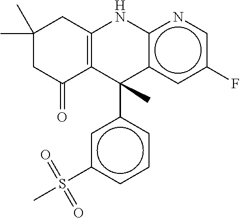 |
| 140R | 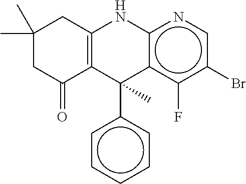 |
| 140S | 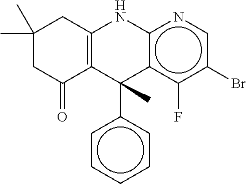 |
| 141R | 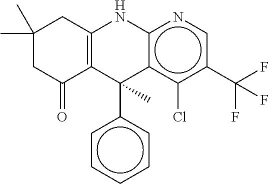 |
| 141S | 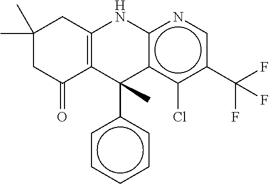 |
| 142R | 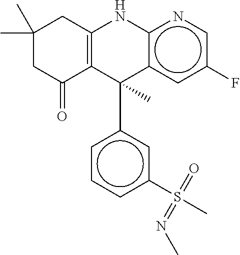 |
| 142S | 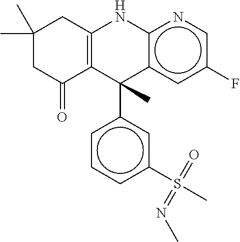 |
| 143R | 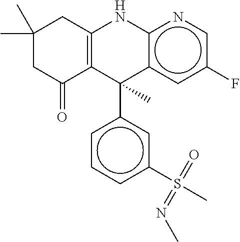 |
| 143S | 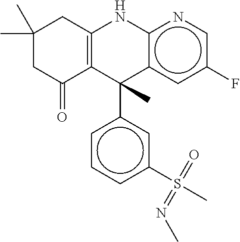 |
| 144R | 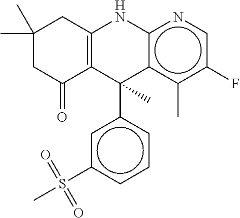 |
| 144S | 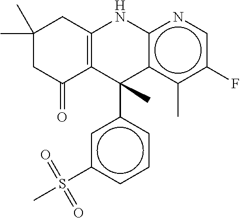 |
| 145R | 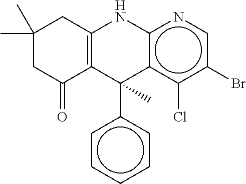 |
| 145S | 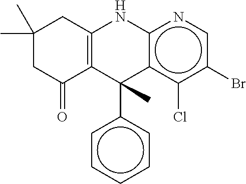 |
| 146R | 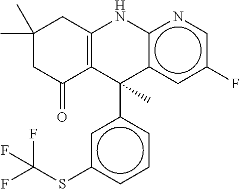 |
| 146S | 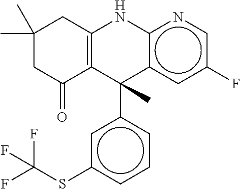 |
| 147R | 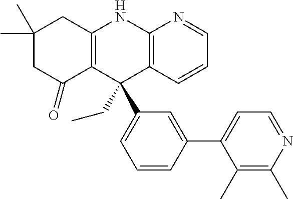 |
| 147S | 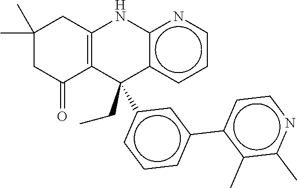 |
| 148R | 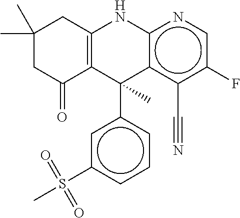 |
| 148S | 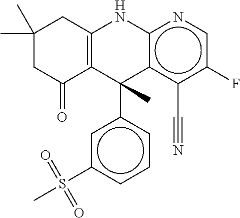 |
| 149R | 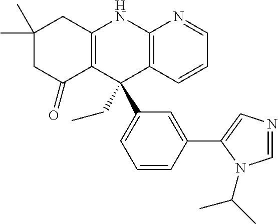 [SMILES: CC[C@]1(c2cccc(-c3cncn3C(C)C)c2)C2=C(CC(C)(C)CC2=O)Nc2ncccc21] |
| 149S | 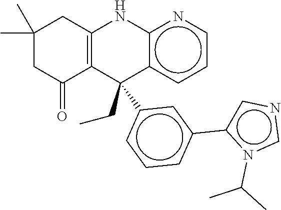 |
| 150R | 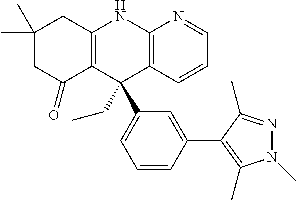 |
| 150S | 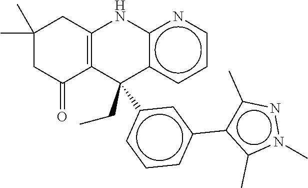 |
| 151R | 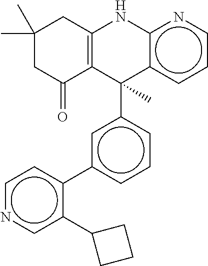 |
| 151S | 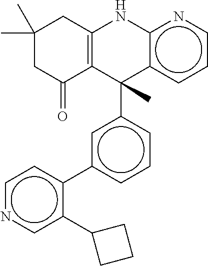 |
| 152R | 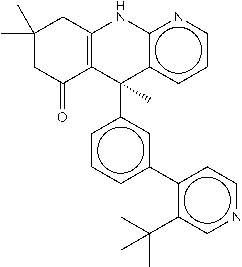 |
| 152S | 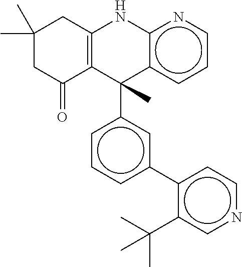 |
| 153R | 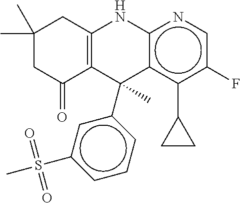 |
| 153S | 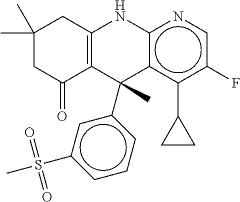 |
| 154R | 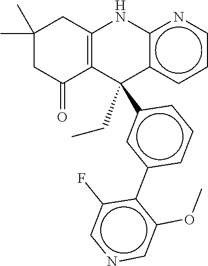 |
| 154S | 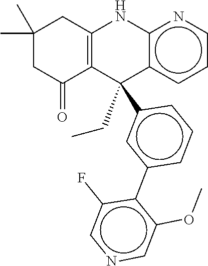 |
| 155R | 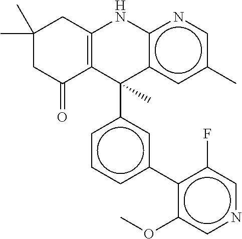 |
| 155S | 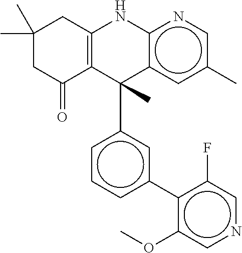 |
| 156R | 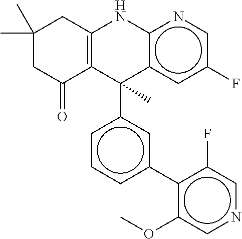 |
| 156S | 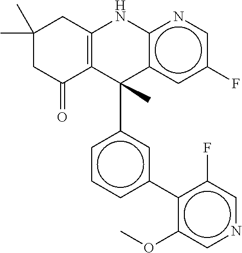 |
| 157R | 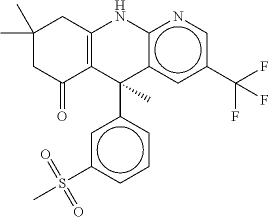 |
| 157S | 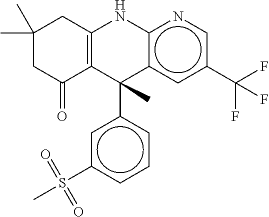 |
| 158R | 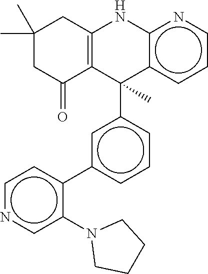 |
| 158S | 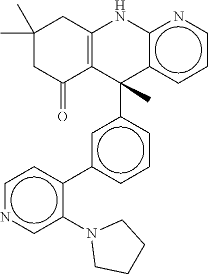 |
| 159R | 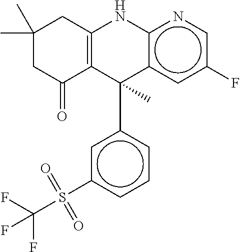 |
| 159S | 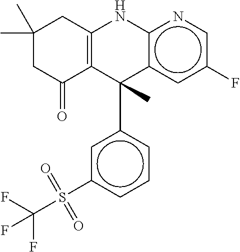 |
| 160R | 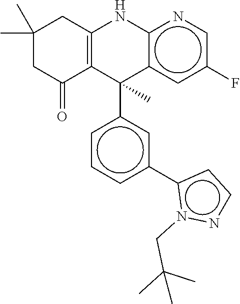 |
| 160S | 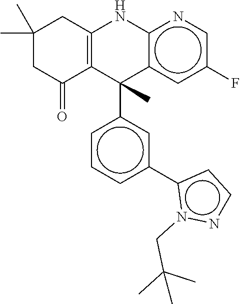 |
| 161R | 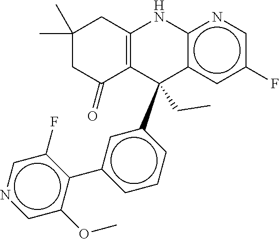 |
| 161S | 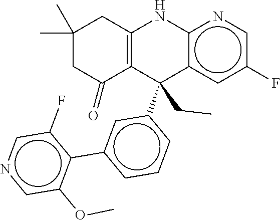 |
| 162R | 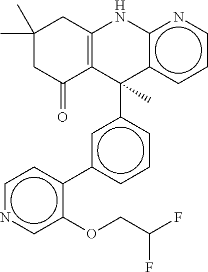 |
| 162S | 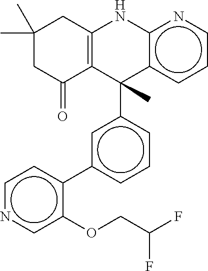 |
| 163R | 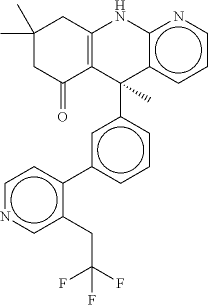 |
| 163S | 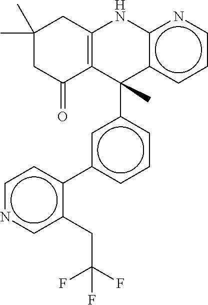 |
| 164R | 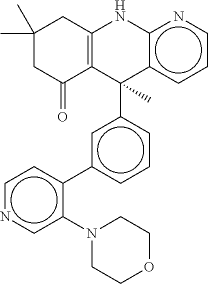 |
| 164S | 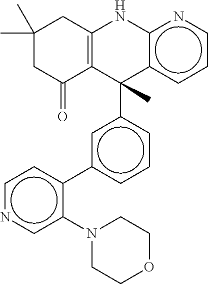 |
| 165R | 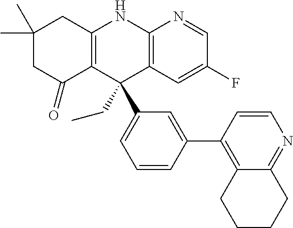 [SMILES: CC[C@]1(c2cccc(-c3ccnc4c3CCCC4)c2)C2=C(CC(C)(C)CC2=O)Nc2ncc(F)cc21] |
| 165S | 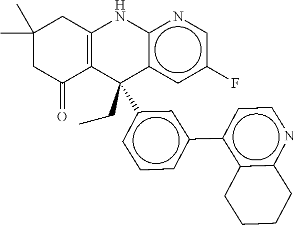 |
| 166R | 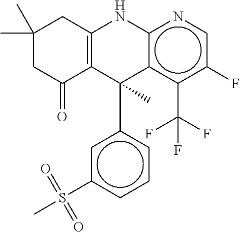 |
| 166S | 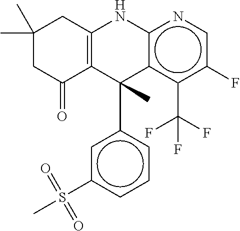 |
| 167R | 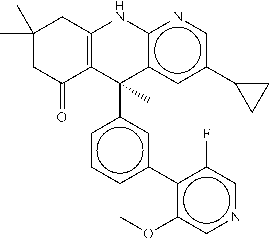 |
| 167S | 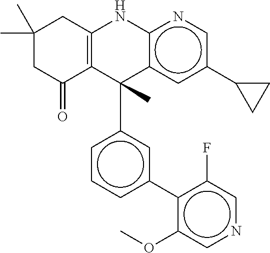 |
| 168R | 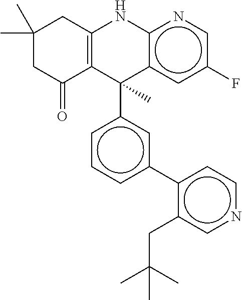 |
| 168S | 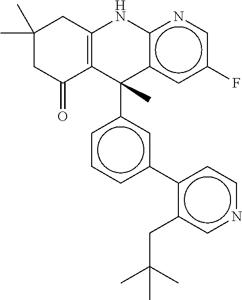 |
| 169R | 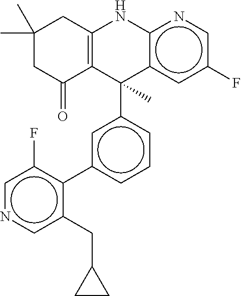 |
| 169S | 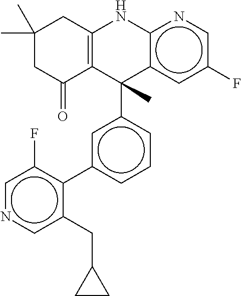 |
| 170R | 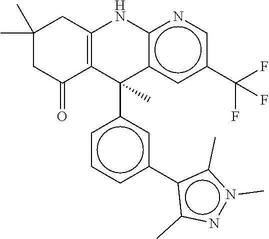 |
| 170S | 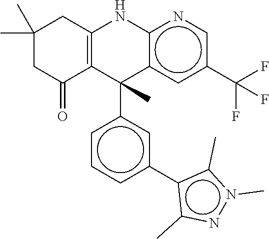 |
| 171R | 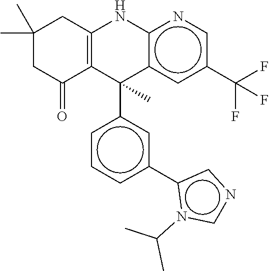 |
| 171S | 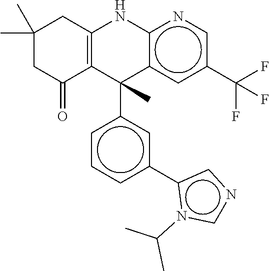 |
| 172R | 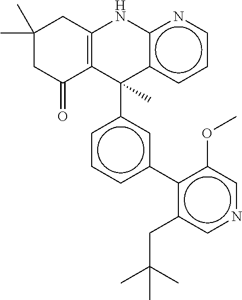 |
| 172S | 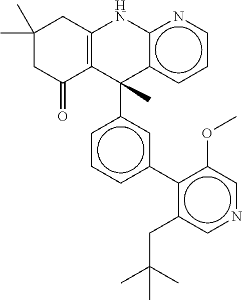 |
| 173R | 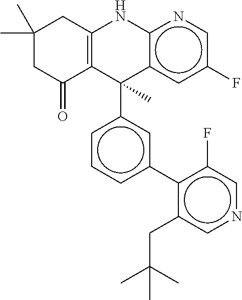 |
| 173S | 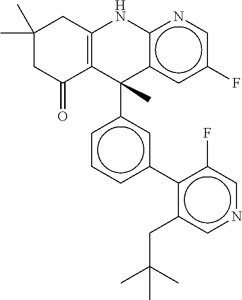 |
| 174R | 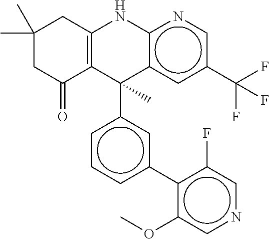 |
| 174S | 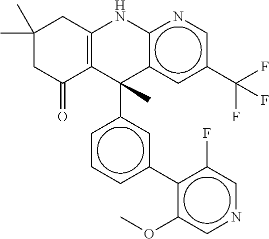 |
| 175R | 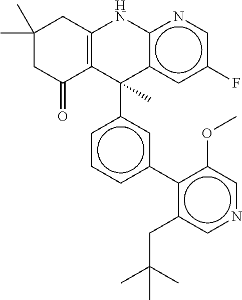 |
| 175S | 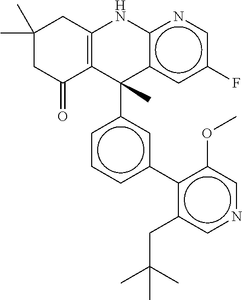 |
| 176R | 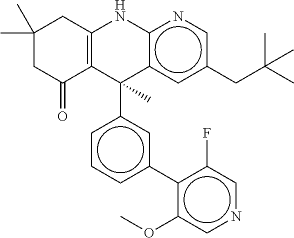 |
| 176S | 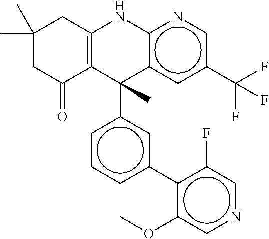 |

or a pharmaceutically acceptable salt, solvate, hydrate, polymorph, co-crystal, tautomer, stereoisomer, isotopically labeled compound, or prodrug thereof.

In some embodiments, the compound is any one of the formulae shown in Table 1A:

TABLE 1A

| No. | Formula |
|---|---|
| 501R | 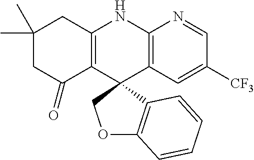 [SMILES: CC1(C)CC(=O)C2=C(C1)Nc1ncc(C(F)(F)F)cc1[C@@]21COc2ccccc21] |
| 501S | 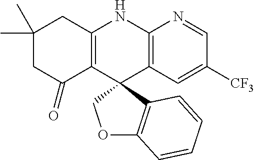 [SMILES: CC1(C)CC(=O)C2=C(C1)Nc1ncc(C(F)(F)F)cc1[C@]21COc2ccccc21] |
| 502R | 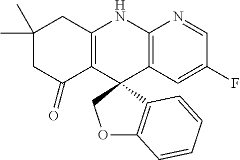 [SMILES: CC1(C)CC(=O)C2=C(C1)Nc1ncc(F)cc1[C@@]21COc2ccccc21] |
| 502S | 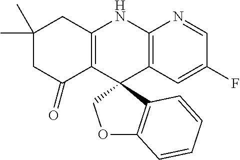 [SMILES: CC1(C)CC(=O)C2=C(C1)Nc1ncc(F)cc1[C@]21COc2ccccc21] |
| 503R | 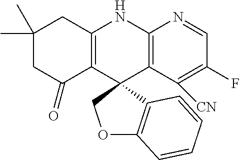 [SMILES: CC1(C)CC(=O)C2=C(C1)Nc1ncc(F)c(C#N)c1[C@@]21COc2ccccc21] |
| 503S | 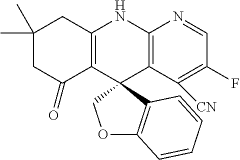 [SMILES: CC1(C)CC(=O)C2=C(C1)Nc1ncc(F)c(C#N)c1[C@]21COc2ccccc21] |
| 504R | 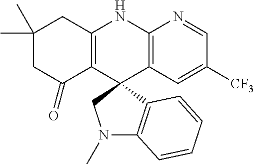 [SMILES: CN1C[C@]2(C3=C(CC(C)(C)CC3=O)Nc3ncc(C(F)(F)F)cc32)c2ccccc21] |
| 504S | 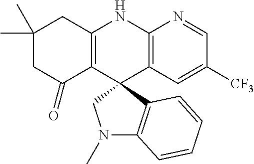 [SMILES: CN1C[C@@]2(C3=C(CC(C)(C)CC3=O)Nc3ncc(C(F)(F)F)cc32)c2ccccc21] |
| 505R | 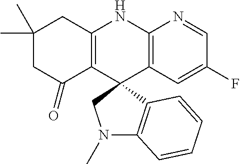 [SMILES: CN1C[C@]2(C3=C(CC(C)(C)CC3=O)Nc3ncc(F)cc32)c2ccccc21] |
| 505S | 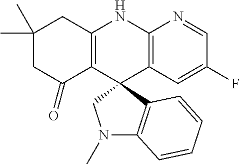 [SMILES: CN1C[C@@]2(C3=C(CC(C)(C)CC3=O)Nc3ncc(F)cc32)c2ccccc21] |
| 506R | 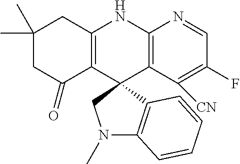 [SMILES: CN1C[C@]2(C3=C(CC(C)(C)CC3=O)Nc3ncc(F)c(C#N)c32)c2ccccc21] |
| 506S | 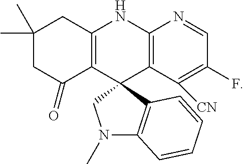 [SMILES: CN1C[C@@]2(C3=C(CC(C)(C)CC3=O)Nc3ncc(F)c(C#N)c32)c2ccccc21] |

or a pharmaceutically acceptable salt, solvate, hydrate, polymorph, co-crystal, tautomer, stereoisomer, isotopically labeled compound, or prodrug thereof.

In some embodiments, the compound is any one of the formulae shown in in Table 1B:

TABLE 1B

| No. | Formula |
|---|---|
| 601R | 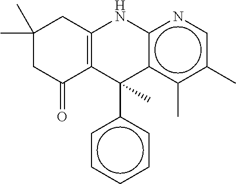 |
| 601S | 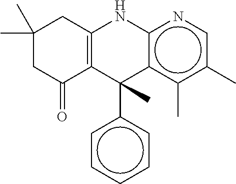 |
| 602R | 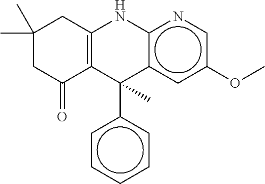 |
| 602S | 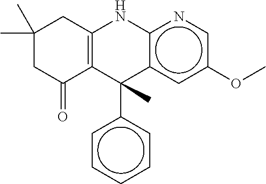 |
| 603R | 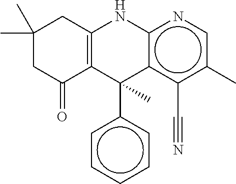 |
| 603S | 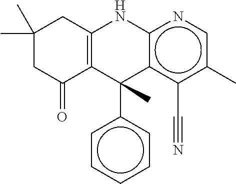 |
| 604R | 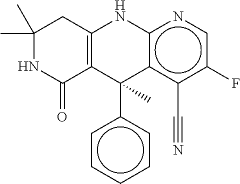 |
| 604S | 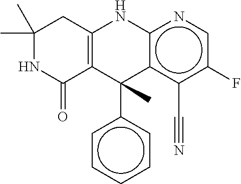 |
| 605R | 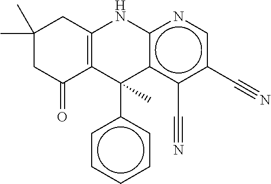 |
| 605S | 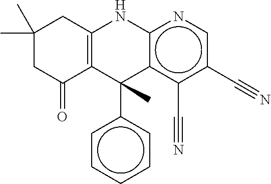 |
| 606R | 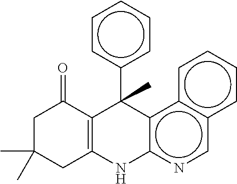 |
| 606S | 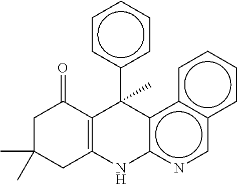 |
| 607R | 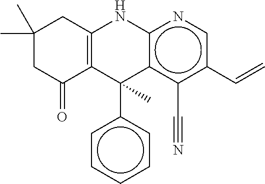 |
| 607S | 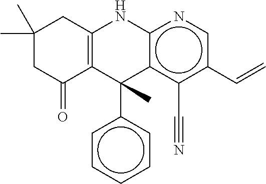 |
| 608R | 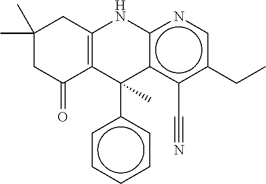 |
| 608S | 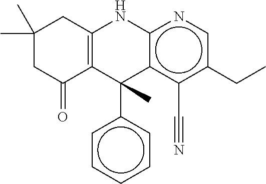 |
| 609R | 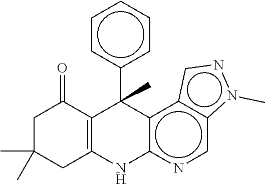 |
| 609S | 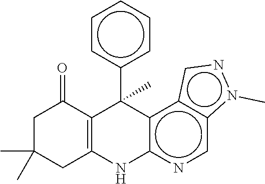 |
| 610R | 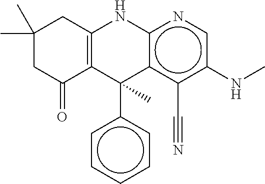 |
| 610S | 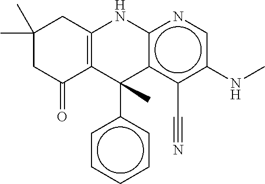 |
| 611R | 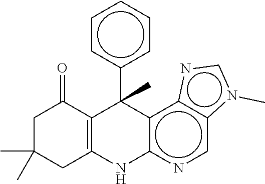 |
| 611S | 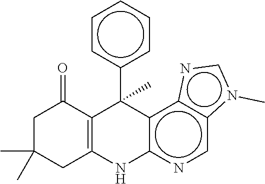 |
| 612R | 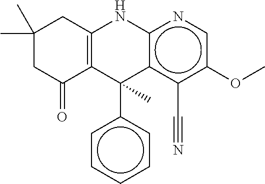 |
| 612S | 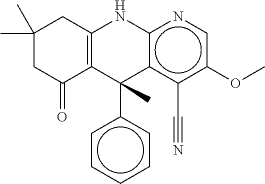 |
| 613R | 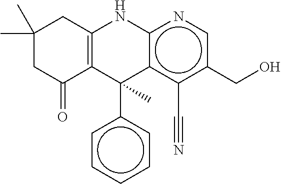 |
| 613S | 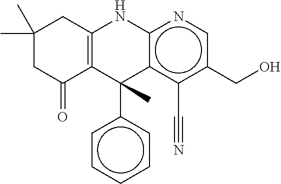 |
| 614R | 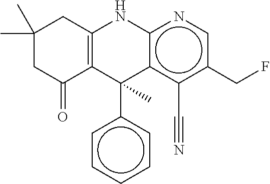 |
| 614S | 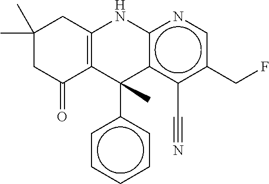 |
| 615R | 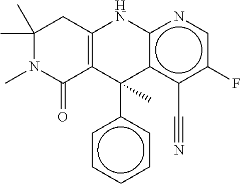 |
| 615S | 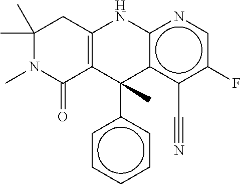 |
| 616RR | 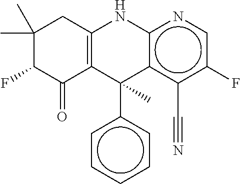 |
| 616SR | 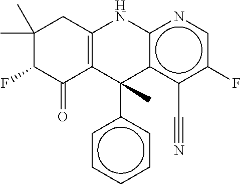 |
| 616RS | 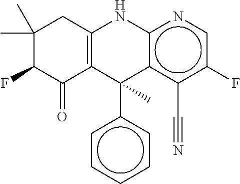 |
| 616SS | 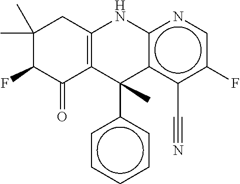 |
| 617RR | 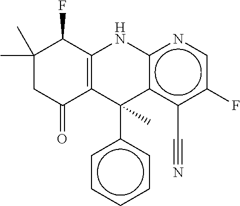 |
| 617SR | 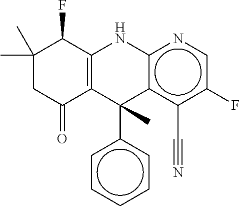 |
| 617RS | 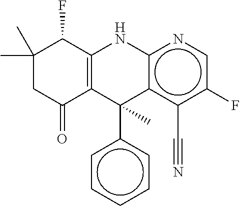 |
| 617SS | 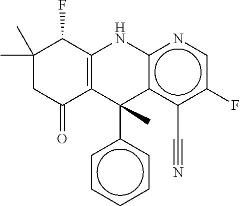 |
| 618R | 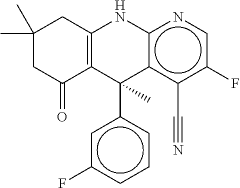 |
| 618S | 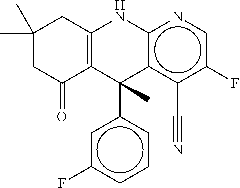 |
| 619R | 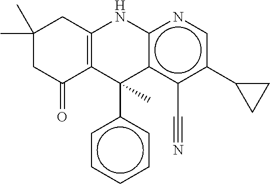 |
| 619S | 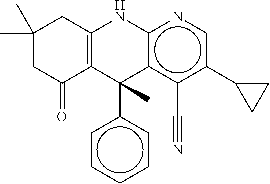 |
| 620R | 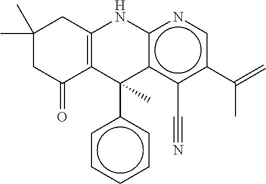 |
| 620S | 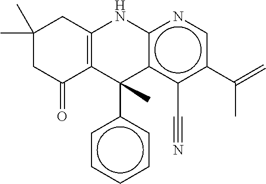 |
| 621R | 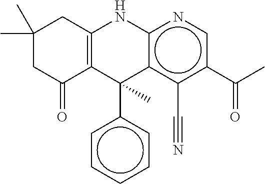 |
| 621S | 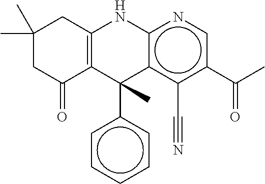 |
| 622R | 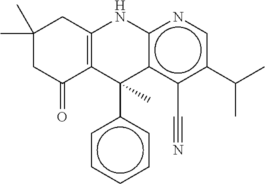 |
| 622S | 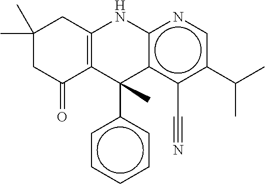 |
| 623R | 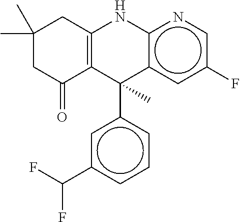 |
| 623S | 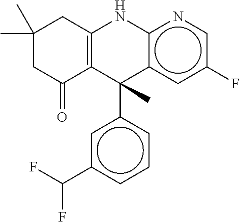 |
| 624R | 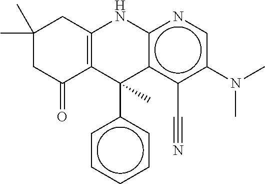 |
| 624S | 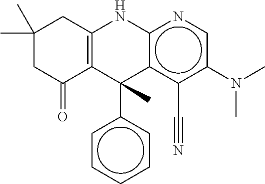 |
| 625R | 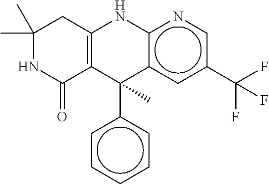 |
| 625S | 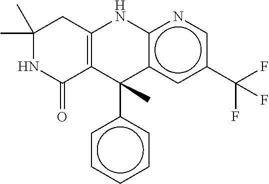 |
| 626R | 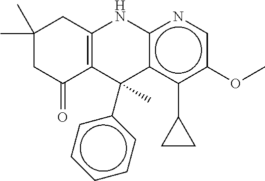 |
| 626S | 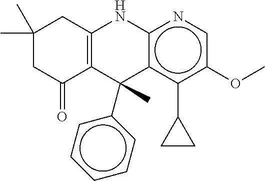 |
| 627R | 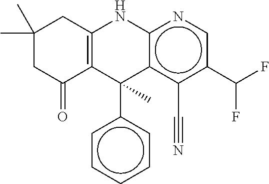 |
| 627S | 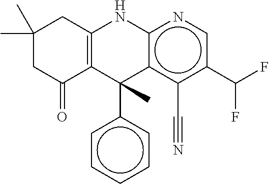 |
| 628R | 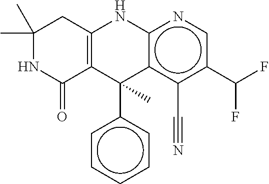 |
| 628S | 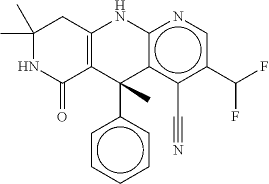 |
| 629R | 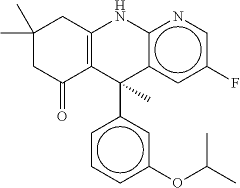 |
| 629S | 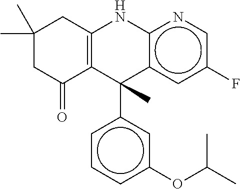 |
| 630R | 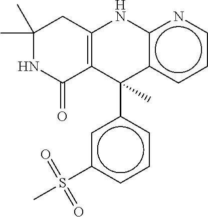 |
| 630S | 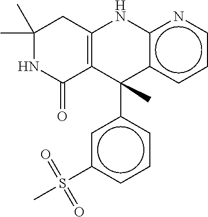 |
| 631R | 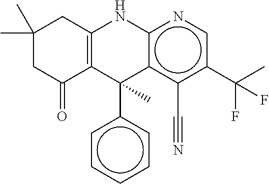 |
| 631S | 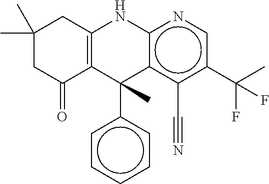 |
| 632R | 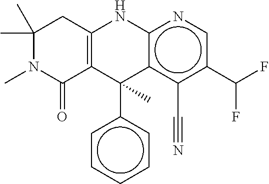 |
| 632S | 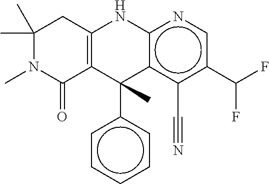 |
| 633RR | 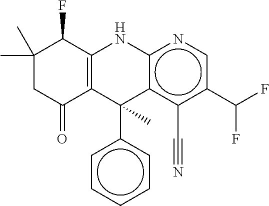 |
| 633SR | 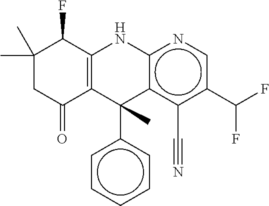 |
| 633RS | 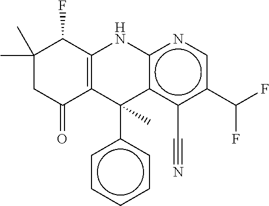 |
| 633SS | 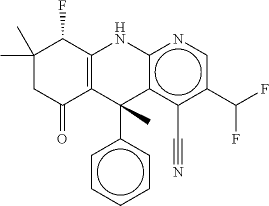 |
| 634R | 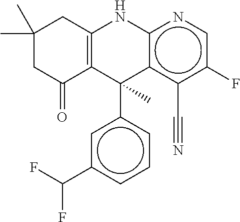 |
| 634S | 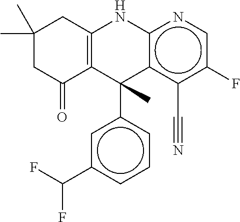 |
| 635R | 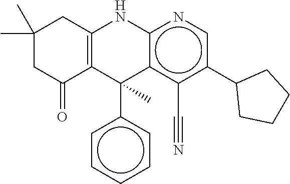 |
| 635S | 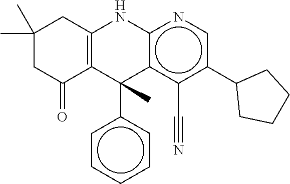 |
| 636R | 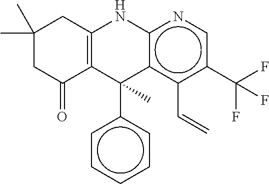 |
| 636S | 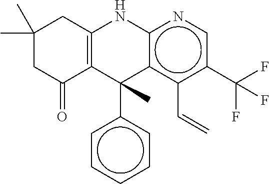 |
| 637R | 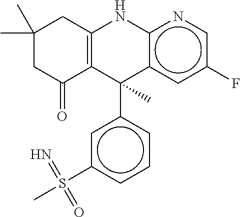 |
| 637S | 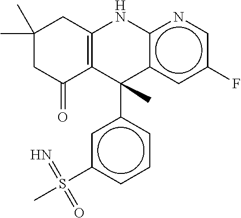 |
| 638R | 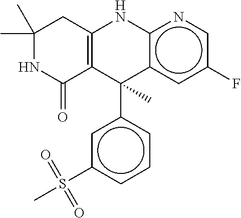 |
| 638S | 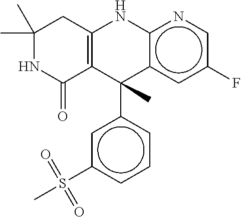 |
| 639R | 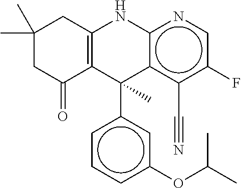 |
| 639S | 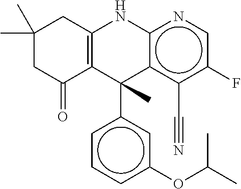 |
| 640R | 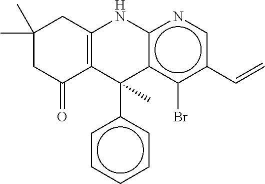 |
| 640S | 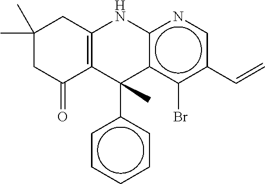 |
| 641R | 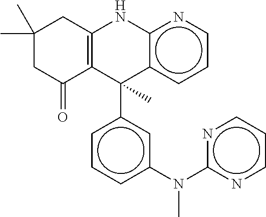 |
| 641S | 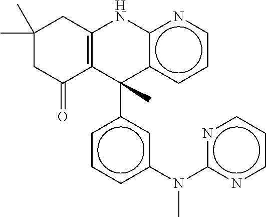 |
| 642R | 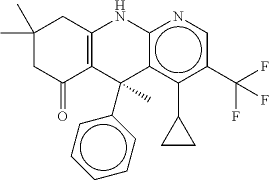 |
| 642S | 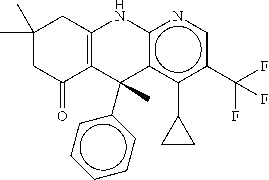 |
| 643R | 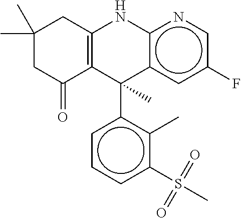 |
| 643S | 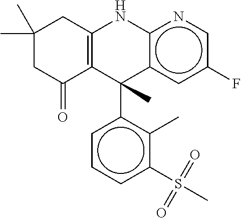 |
| 644R | 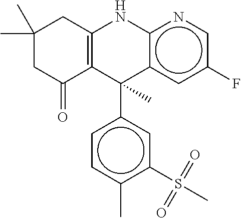 |
| 644S | 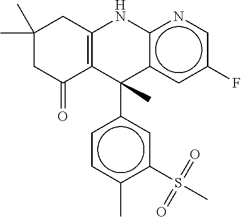 |
| 645R | 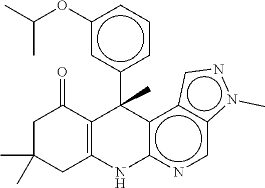 |
| 645S | 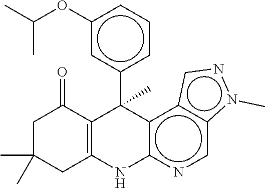 |
| 646R | 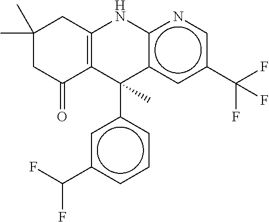 |
| 646S | 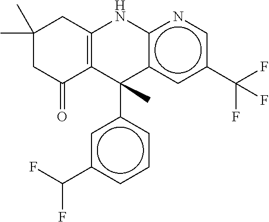 |
| 647R | 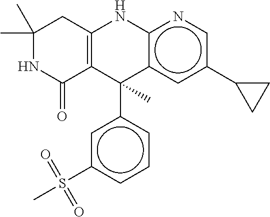 |
| 647S | 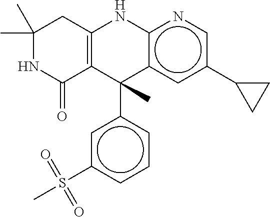 |
| 648R | 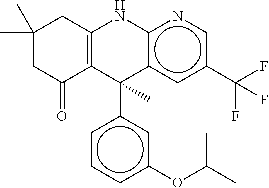 |
| 648S | 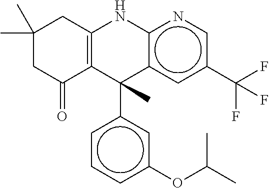 |
| 649R | 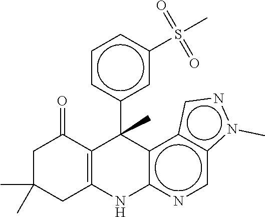 |
| 649S | 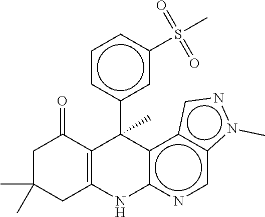 |
| 650R | 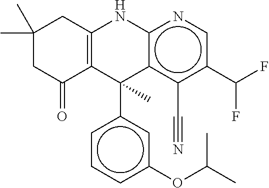 |
| 650S | 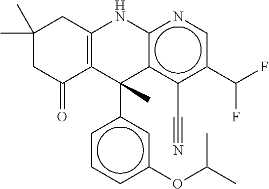 |
| 651R | 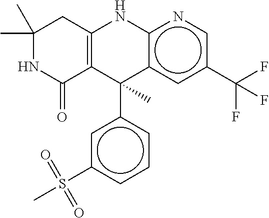 |
| 651S | 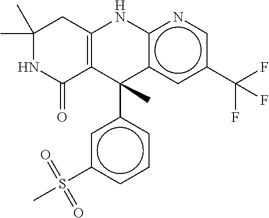 |
| 652R | 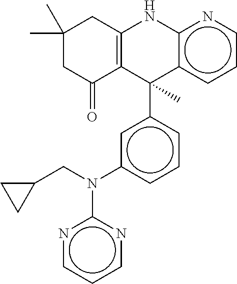 |
| 652S | 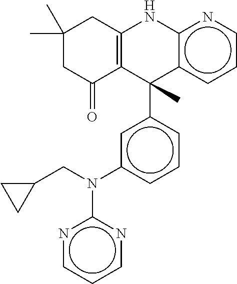 |
| 653R | 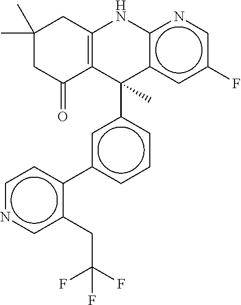 |
| 653S | 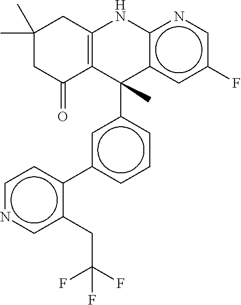 |
| 654RR | 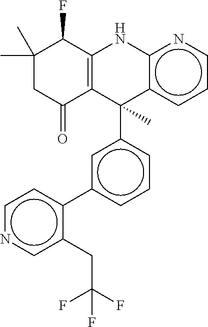 |
| 654SR | 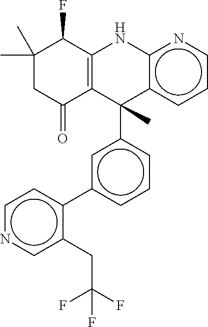 |
| 654RS | 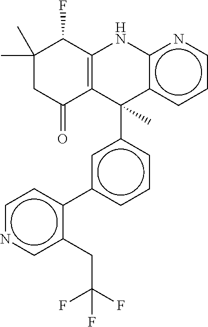 |
| 654SS | 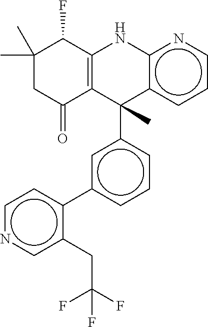 |
| 655RS | 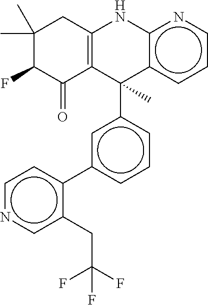 |
| 655SS | 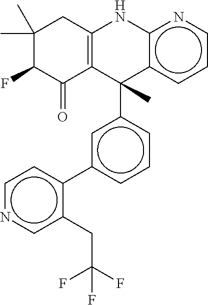 |
| 655RR | 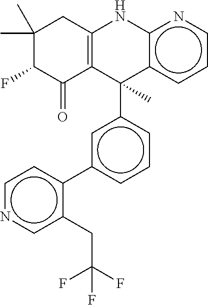 |
| 655SR | 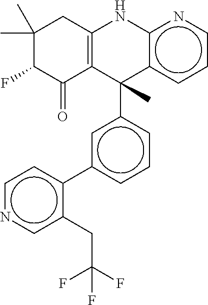 |
| 656R | 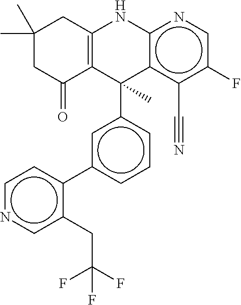 |
| 656S | 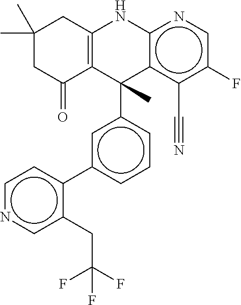 |
| 657R | 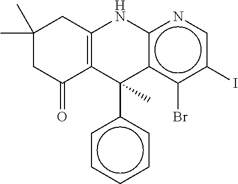 |
| 657S | 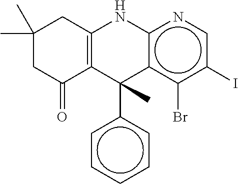 |

or a pharmaceutically acceptable salt, solvate, hydrate, polymorph, co-crystal, tautomer, stereoisomer, isotopically labeled compound, or prodrug thereof.

In some embodiments, the compound is any one of the formulae shown in Table 2:

TABLE 2

| No. | Formula |
|---|---|
| 1002R | 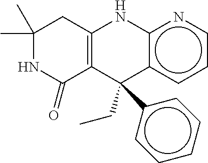 |
| 1002S | 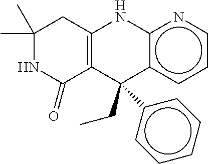 |
| 1004R | 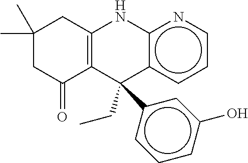 |
| 1004S | 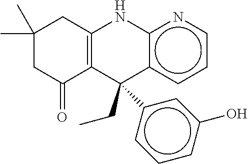 |
| 1006RR | 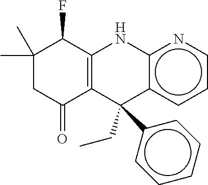 |
| 1006SR | 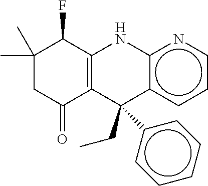 |
| 1006RS | 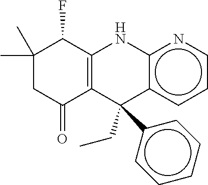 |
| 1006SS | 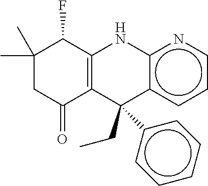 |
| 1007R | 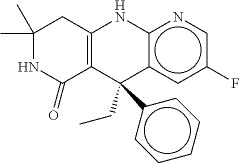 |
| 1007S | 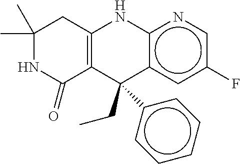 |
| 1008R | 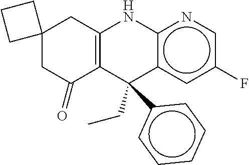 |
| 1008S | 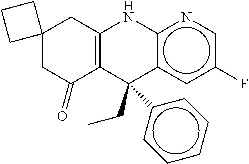 |
| 1009RR | 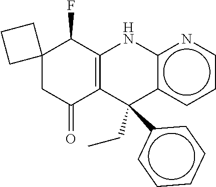 |
| 1009SR | 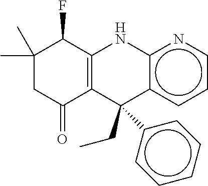 |
| 1009RS | 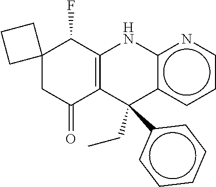 |
| 1009SS | 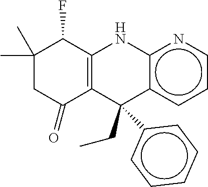 |
| 1010R | 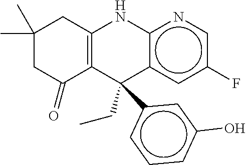 |
| 1010S | 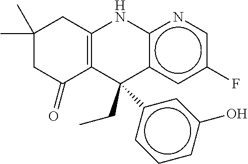 |
| 1011R | 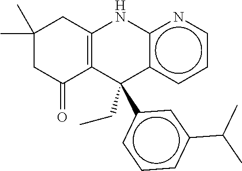 |
| 1011S | 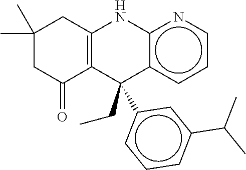 |
| 1012R | 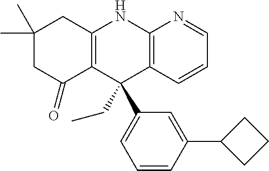 [SMILES: CC[C@]1(c2cccc(C3CCC3)c2)C2=C(CC(C)(C)CC2=O)Nc2ncccc21] |
| 1012S | 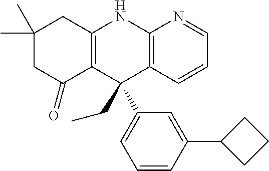 [SMILES: CC[C@@]1(c2cccc(C3CCC3)c2)C2=C(CC(C)(C)CC2=O)Nc2ncccc21] |
| 1014R | 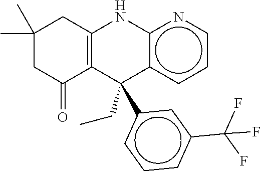 |
| 1014S | 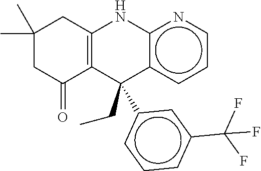 |
| 1015R | 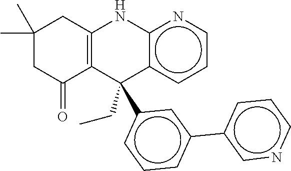 |
| 1015S | 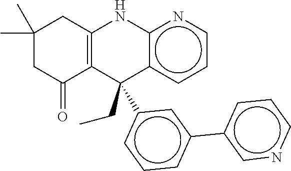 |
| 1016R | 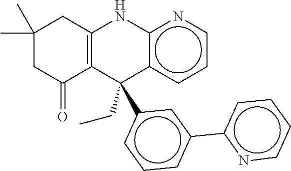 |
| 1016S | 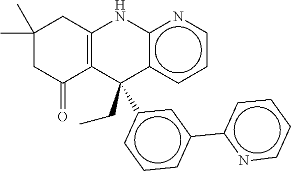 |
| 1017R | 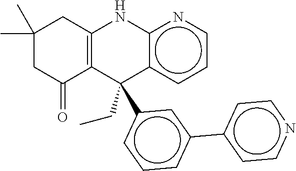 |
| 1017S | 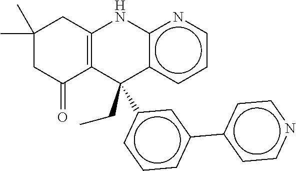 |
| 1018R | 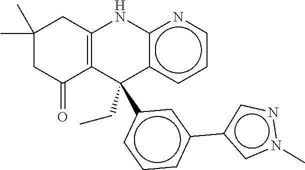 |
| 1018S | 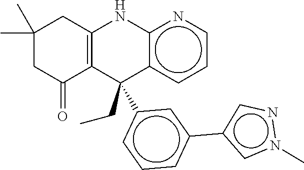 |
| 1019R | 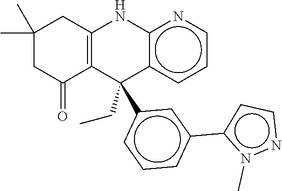 |
| 1019S | 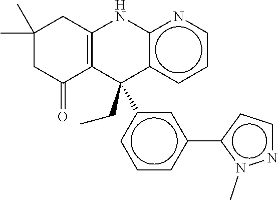 |
| 1020R | 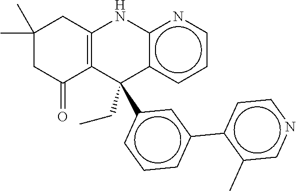 |
| 1020S | 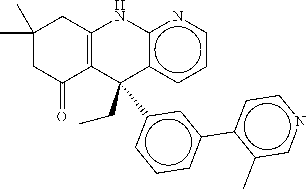 |
| 1021R | 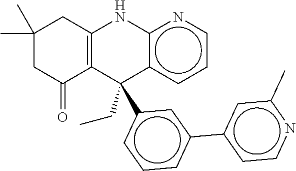 |
| 1021S | 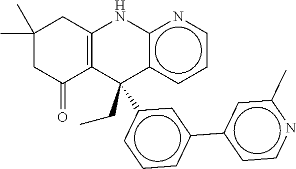 |
| 1022R | 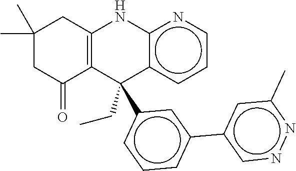 |
| 1022S | 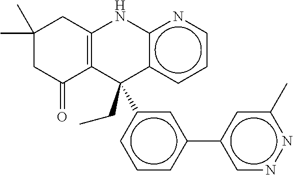 |
| 1023R | 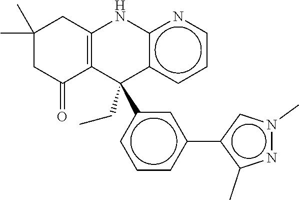 |
| 1023S | 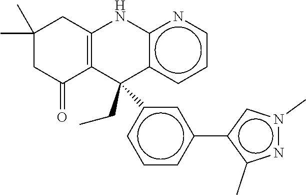 |
| 1024R | 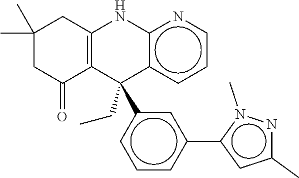 |
| 1024S | 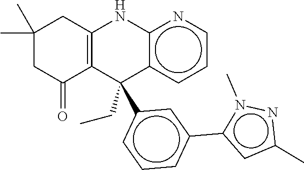 |
| 1025R | 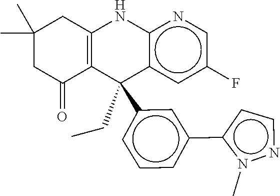 |
| 1025S | 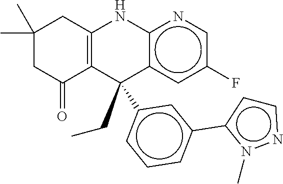 |
| 1027R | 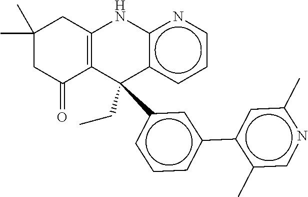 |
| 1027S | 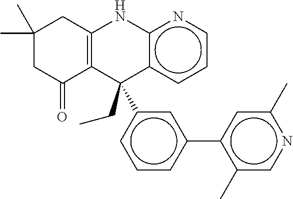 |
| 1028R | 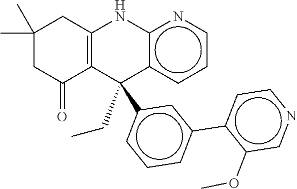 |
| 1028S | 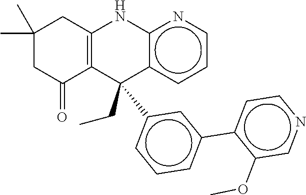 |
| 1031R | 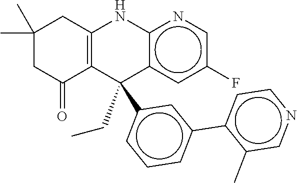 |
| 1031S | 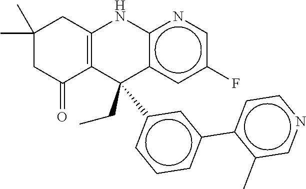 |
| 1032R | 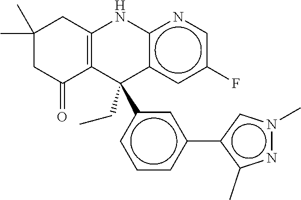 |
| 1032S | 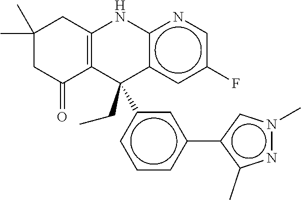 |
| 1033R | 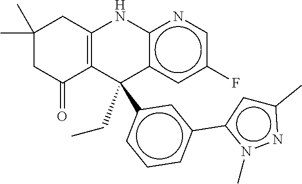 |
| 1033S | 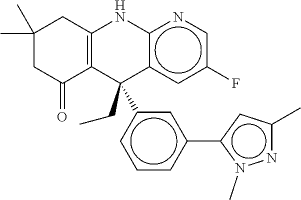 |
| 1034R | 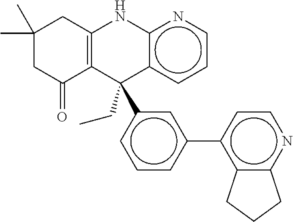 |
| 1034S | 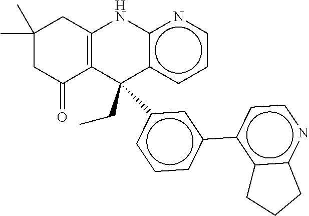 |
| 1035R | 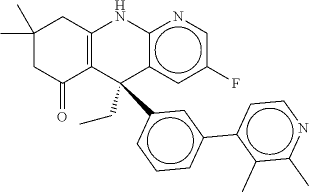 |
| 1035S | 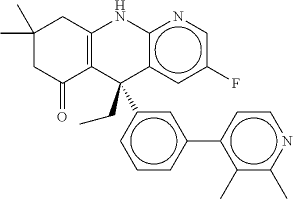 |
| 1037R | 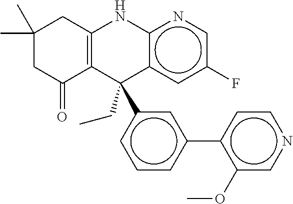 |
| 1037S | 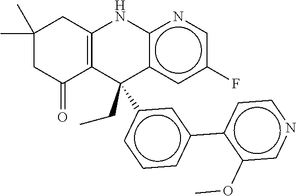 |
| 1038R | 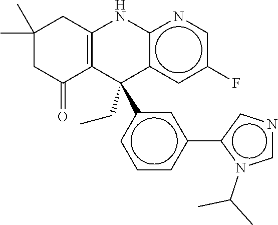 |
| 1038S | 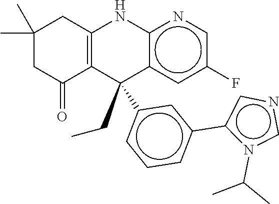 |
| 1039R | 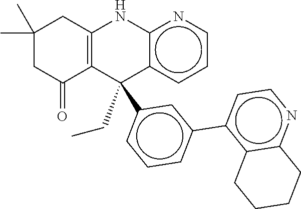 |
| 1039S | 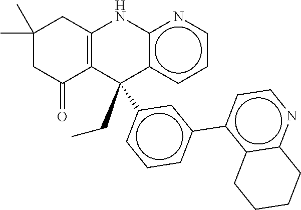 |

“A compound of the present disclosure” or “a compound provided herein” refers to a compound of Formula I (e.g., a compound shown in Table 1 or 2), or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula I, or a pharmaceutically acceptable salt, solvate, hydrate, polymorph, co-crystal, tautomer, stereoisomer, or isotopically labeled compound thereof. In some embodiments, a compound of the present disclosure is a compound of Formula I, or a pharmaceutically acceptable salt, tautomer, stereoisomer, or isotopically labeled compound thereof. In some embodiments, a compound of the present disclosure is a compound of Formula I, or a pharmaceutically acceptable salt thereof.

Pharmaceutical Compositions. Kits, and Administration

In another aspect, the present disclosure provides pharmaceutical compositions comprising a compound provided herein and optionally a pharmaceutically acceptable excipient. In some embodiments, the pharmaceutical composition further comprises one or more additional pharmaceutical agents.

Pharmaceutical compositions can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

In some embodiments, pharmaceutical compositions are prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers. (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzil alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor® alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, in some embodiments, delayed absorption of a parenterally administered drug form is accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

In some embodiments, the compounds and compositions provided herein are administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound, mode of administration, and the like. In some embodiments, an effective amount is included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. In some embodiments, the amount to be administered to, for example, a child or an adolescent is determined by a medical practitioner or person skilled in the art. In some embodiments, the amount to be administered to, for example, a child or an adolescent is lower or the same as that administered to an adult.

In some embodiments, a compound or composition, as described herein, is administered in combination with one or more additional pharmaceutical agents (e.g., therapeutically and/or prophylactically active agents). In some embodiments, the compounds or compositions are administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity and/or production of a GSK3 in a subject, cell, tissue, or biological sample), improve bioavailability, improve safety, reduce drug resistance, reduce and/or modify metabolism, inhibit excretion, and/or modify distribution in a subject, cell, tissue, or biological sample. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

In some embodiments, the compound or composition is administered concurrently with, prior to, or subsequent to one or more additional pharmaceutical agents. In some embodiments, the one or more additional pharmaceutical agents are useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease. In some embodiments, each additional pharmaceutical agent is administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds or pharmaceutical compositions described herein are administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

Also encompassed by the disclosure are kits (e.g., pharmaceutical packs) comprising a compound or pharmaceutical composition provided herein; and instructions for using the compound or pharmaceutical composition provided herein. In some embodiments, the kit comprises a pharmaceutical composition or compound described herein and a container (e.g., a vial, ampule, bottle, syringe, and/or dispenser package, or other suitable container). In some embodiments, provided kits optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

In certain embodiments, the kit includes a first container comprising a compound or pharmaceutical composition provided herein. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof. In certain embodiments, the kits are useful for inhibiting the activity (e.g., aberrant activity, such as increased activity) and/or production of a GSK3 in a subject, cell, tissue, or biological sample.

In certain embodiments, a kit provided herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. In certain embodiments, the kits and instructions provide for inhibiting the activity (e.g., aberrant activity, such as increased activity) and/or production of a GSK3 in a subject, cell, or tissue. A kit described herein may include one or more additional pharmaceutical agents described herein as a separate composition.

### Methods of Treatment and Uses

GSK3 remains a therapeutic target of interest for many diseases, however the development of small molecule inhibitors has been hindered by significant safety concerns related to β-catenin activation and the lack of structural information for the GSK3α paralog.

Originally identified as a protein kinase involved in the regulation of glycogen metabolism, GSK3 is now known to be a multi-functional protein with key roles in diverse biological processes including cell proliferation, differentiation, apoptosis, embryonic development, and insulin response (Cole A R, 2012; Racaud-Sultan C, Vergnolle N, 2021; Henriksen E J and Dokken B B, 2006). In addition, GSK3 is also of considerable interest as a therapeutic target because of its involvement in core pathophysiologies underlying multiple diseases including Alzheimer's Disease (AD), Fragile X Syndrome, diabetes, and several types of cancer (Bhat R V et al., 2004; Beurel et al., 2015; O'Leary O and Nolan Y, 2015; McCoubrey J A et al., 2014).

GSK3 has been of particular focus in AD because preclinical data demonstrates that modulation of this kinase has beneficial effects on both hallmark pathological processes in AD: hyperphosphorylation of tau protein and production of amyloid-beta peptides (Phiel, C J et al., 2003; Plattner, F et al., 2006; Ly, P et al., 2013). Based on the therapeutic potential of targeting GSK3 in AD, several GSK3 inhibitors have advanced into clinical trials (Georgievska B et al., 2013; del Ser T et al., 2013), but these compounds have had limited clinical success due to lack of efficacy and/or significant safety concerns. Safety concerns around GSK3 stem from evidence demonstrating a key role for this kinase in regulating the Wnt-β-catenin pathway (Behrens J et al., 1998) and multiple reports have demonstrated that long-term inhibition of GSK3 can induce aberrant proliferation and hyperplasia in several tissues in vivo (Meijer et al., 2004; Sato N et al., 2004; Coghlan et al., 2000; Bhat R et al., 2003).

GSK3 exists as two paralogs, GSK3α and GSK30, which are encoded from separate genes and are thought to have arisen evolutionarily through gene duplication. The GSK3 paralogs are similar in sequence with 95% identity in the ATP binding site (67% amino acid identity overall) and exhibit a high degree of overlap in both tissue expression patterns and in their phosphorylation substrates (Woodgett, J R 1991; Yao H B et al., 2002; Soutar et al., 2010; Kaidanovich-Beilin O, Woodgett J R 2011). Interestingly, recent studies have highlighted distinct functional roles for GSK3α in several biological processes (Beurel et al., 2015). Notably, loss of GSK3β causes embryonic lethality in mice whereas loss of GSK3α results in relatively modest defects (Kim, W Y et al., 2009; Morgansmith, M et al., 2014). In the context of AD, most of the focus has traditionally been on GSK3β (Hooper C et al., 2008). However, in a study using a mouse model that combines amyloid and tau pathologies, Hurtado et al. (2012) reported that knockdown of either GSKα or GSK3β ameliorated tau hyper-phosphorylation, yet only knockdown of GSK3α additionally reduced amyloid pathology. More recently, selective loss or inhibition of GSK3α, but not GSK30, has been shown to rescue deficits in a mouse model of Fragile x (McCamphill P K et al., 2020) and suppress tumorigenesis in models of acute myeloid leukemia (Wang Y et al., 2019). With respect to safety risks, studies have shown that selective reduction or ablation of either GSK3 paralog circumvents β-catenin stabilization (Doble B W et al., 2007) and that selective genetic suppression of GSK3α impairs leukemia progression in mouse models of AML without increasing β-catenin levels (Banerji V et al., 2012).

The challenge in discovery of selective GSK3 inhibitors is predominantly due to the high degree of homology in the ATP binding site where the primary difference is an Asp to Glu switch located in the hinge region (Wagner F F et al., 2018). This difference is further complicated by the positioning of corresponding amino acid side chains located outside of the ATP binding site and therefore directed away from potential interactions with ATP competitive inhibitors. Previously a series of oxadiazole inhibitors were identified that were able to achieve ˜3-fold GSK3 selectivity for the −α paralog (Lo Monte et al., 2013) and there have also been reports of imide-based (Palomo V et al., 2012) and thioxoimidazolidine kinase inhibitors that exhibit paralog selectivity up to ˜7-fold (Wang Y et al., 2019). Interestingly, a series of aminopyrazole inhibitors with up to 8-fold selectivity were recently reported by Wagner et al., (2018) and their development was based on the crystal structure of a GSK3β D133E mutant thought to mimic key differences between the two paralogs within the ATP binding site. Further results from this study also identified differential hydrogen bonding networks outside of the hinge Asp/Glu switch.

The compounds and pharmaceutical compositions provided herein may be useful in a provided method. Compounds useful in a provided method and pharmaceutical compositions provided herein may be useful for the inhibition of a GSK3. Without being bound by any particular theory, the compounds useful in a provided method and the pharmaceutical compositions provided herein being useful as described herein may be at least in part due to their inhibition of the activity and/or production of a GSK3. Compared to known GSK3 inhibitors, the compounds useful in a provided method and the pharmaceutical compositions provided herein may increase the potency, efficacy, and/or selectivity in inhibiting the activity and/or production of a GSK3 in a subject, cell, or tissue. Compared to known GSK3 inhibitors, the compounds useful in a provided method and the pharmaceutical compositions provided herein may increase bioavailability, safety, and/or therapeutic window, reduce toxicity and/or resistance, and/or increase subject compliance, in a subject. In certain embodiments, the compounds useful in a provided method are selective GSK3 inhibitors (e.g., GSK3 inhibitors that selectively inhibit a GSK3 over one or more other kinases). In certain embodiments, the compounds useful in a provided method are selective GSK3α inhibitors (e.g., GSK3α inhibitors that selectively inhibit GSK3α over GSK3β and optionally one or more other kinases). In certain embodiments, the compounds useful in a provided method are selective GSK3β inhibitors (e.g., GSK3β inhibitors that selectively inhibit GSK3β over GSK3α and optionally one or more other kinases).

In some embodiments, the GSK3 is glycogen synthase kinase 3 α (GSK3α). In some embodiments, the compound provided herein is more selective for inhibiting the activity and/or production of GSK3α than glycogen synthase kinase 3 β (GSK3β) in an in vitro assay. In some embodiments, the selectivity of the compound provided herein for GSK3α over GSK3β is determined by the relative K_{d} values for GSK3α and GSKβ. In certain embodiments, the selectivity of the compound provided herein for GSK3α over GSK3β is determined by the quotient of the IC₅₀ value of the compound in inhibiting GSK3β over the IC₅₀ value of the compound in inhibiting GSK3α. In certain embodiments, the selectivity of the compound provided herein for GSK3α over GSK3β is at least 1.1, at least 1.3, at least 1.5, at least 1.7, at least 2, at least 3, at least 4, at least 6, at least 8, at least 10, at least 12, at least 15, at least 17, at least 20, at least 30, at least 50, at least 100, at least 1,000, or at least 10,000. In certain embodiments, the selectivity of the compound provided herein for GSK3α over GSK3β is at least 3. In certain embodiments, the compound provided herein is at least three times more selective for inhibiting the activity and/or production of GSK3α than GSK3β in an in vitro assay. In certain embodiments, the selectivity of the compound provided herein for GSK3α over GSK3β is at least 8. In certain embodiments, the selectivity of the compound provided herein for GSK3α over GSK3β is at least 10. In certain embodiments, the selectivity of the compound provided herein for GSK3α over GSK3β is at least 12. In certain embodiments, the selectivity of the compound provided herein for GSK3α over GSK3β is at least 15. In certain embodiments, the selectivity of the compound provided herein for GSK3α over GSK3β is at least 17. In certain embodiments, the selectivity of the compound provided herein for GSK3α over GSK3β is at least 20. In certain embodiments, the selectivity of the compound provided herein for GSK3α over GSK3β is at least 30. In certain embodiments, the selectivity of the compound provided herein for GSK3α over GSK3β is at least 50. In certain embodiments, the selectivity of the compound provided herein for GSK3α over GSK3β is at least 100. In certain embodiments, the selectivity of the compound provided herein for GSK3α over GSK3β is between 10 and 1,000, inclusive.

In some embodiments, the GSK3 is glycogen synthase kinase 3 β (GSK3β). In some embodiments, the compound provided herein is more selective for inhibiting the activity and/or production of GSK3β than glycogen synthase kinase 3 α (GSK3α) in an in vitro assay. In some embodiments, the selectivity of the compound provided herein for GSK3β over GSK3α is determined by the relative K_{d} values for GSK3β and GSKα. In certain embodiments, the selectivity of the compound provided herein for GSK3β over GSK3α is determined by the quotient of the IC₅₀ value of the compound in inhibiting GSK3α over the IC₅₀ value of the compound in inhibiting GSK3β. In certain embodiments, the selectivity of the compound provided herein for GSK3β over GSK3α is at least 1.1, at least 1.3, at least 1.5, at least 1.7, at least 2, at least 3, at least 4, at least 6, at least 8, at least 10, at least 12, at least 15, at least 17, at least 20, at least 30, at least 50, at least 100, at least 1,000, or at least 10,000. In certain embodiments, the selectivity of the compound provided herein for GSK3β over GSK3α is at least 3. In certain embodiments, the compound provided herein is at least three times more selective for inhibiting the activity and/or production of GSK3β than GSK3α in an in vitro assay. In certain embodiments, the selectivity of the compound provided herein for GSK3β over GSK3α is at least 8. In certain embodiments, the selectivity of the compound provided herein for GSK30 over GSK3α is at least 10. In certain embodiments, the selectivity of the compound provided herein for GSK3β over GSK3α is at least 12. In certain embodiments, the selectivity of the compound provided herein for GSK3β over GSK3α is at least 15. In certain embodiments, the selectivity of the compound provided herein for GSK3β over GSK3α is at least 17. In certain embodiments, the selectivity of the compound provided herein for GSK3D over GSK3α is at least 20. In certain embodiments, the selectivity of the compound provided herein for GSK3β over GSK3α is at least 30. In certain embodiments, the selectivity of the compound provided herein for GSK3β over GSK3α is at least 50. In certain embodiments, the selectivity of the compound provided herein for GSK3β over GSK3α is at least 100. In certain embodiments, the selectivity of the compound provided herein for GSK3β over GSK3α is between 10 and 1,000, inclusive.

In another aspect, provided herein is a method of treating a disease in a subject in need thereof, the method comprising administering to the subject an effective amount of: a compound useful in a provided method or a pharmaceutical composition provided herein. In another aspect, provided herein is a use a compound useful in a provided method or pharmaceutical composition provided herein for the manufacture of a medicament for treating a disease in a subject in need thereof. In another aspect, provided herein is a compound useful in a provided method or pharmaceutical composition provided herein for use in treating a disease in a subject in need thereof.

In another aspect, provided herein is a method of preventing a disease in a subject in need thereof, the method comprising administering to the subject an effective amount of; a compound useful in a provided method or a pharmaceutical composition provided herein. In another aspect, provided herein is a use of a compound useful in a provided method or pharmaceutical composition provided herein for the manufacture of a medicament for preventing a disease in a subject in need thereof. In another aspect, provided herein is a compound useful in a provided method or pharmaceutical composition provided herein for use in preventing a disease in a subject in need thereof.

In some embodiments the effective amount is further effective in inhibiting the activity and/or production of a GSK3. In some embodiments, the effective amount is further effective in inhibiting the aberrantly high activity and/or production of the GSK3. In some embodiments, the effective amount is a therapeutically effective amount. In some embodiments, the effective amount is a prophylactically effective amount. In certain embodiments, the effective amount is an amount effective for treating a disease in a subject in need thereof. In certain embodiments, the effective amount is an amount effective for preventing a disease in a subject in need thereof. In certain embodiments, the effective amount is an amount effective for reducing the risk of developing a disease in a subject in need thereof. In certain embodiments, the effective amount is an amount effective for inhibiting the activity (e.g., aberrant activity, such as increased activity) of a GSK3 in a subject, cell, tissue, or biological sample. In certain embodiments, the effective amount is an amount effective for inhibiting the production of a GSK3 in a subject, cell, tissue, or biological sample.

In certain embodiments, the effective amount is an amount effective for inhibiting the activity of a GSK3 by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of a GSK3 by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of a GSK3 by a range between a percentage described in this paragraph and another percentage described in this paragraph, inclusive.

In some embodiments, the term refers to a reduction of the level of production, e.g., GSK3 protein production, to a level that is statistically significantly lower than an initial level, which may, for example, be a baseline level of production. In some embodiments, the term refers to a reduction of the level of production, e.g., GSK3 protein production, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of production.

In some embodiments, the disease is associated with aberrantly high activity and/or production of a GSK3. In some embodiments, the disease is associated with aberrantly high activity and/or production of catenin beta-1. In some embodiments, the disease is associated with a mutation and/or overexpression of the CTNNB1 gene. In some embodiments, the disease is a central nervous system disease. In some embodiments, the disease is a brain disease. In some embodiments, the disease is Edwards syndrome, gliosis, hyperekplexia, Meckel syndrome, myoclonic epilepsy myopathy sensory ataxia, narcolepsy, prion diseases, serotonin syndrome, or spinal cord disease. In some embodiments, the disease is a mental or behavioral disease. In some embodiments, the disease is CTNNB1 syndrome. In some embodiments, the disease is fragile X syndrome. In some embodiments, the disease is autism. In some embodiments, the disease is schizophrenia. In some embodiments, the disease is bipolar disorder. In some embodiments, the disease is attention deficit hyperactivity disorder. In some embodiments, the disease is a neurological disease. In some embodiments, the disease is seizure. In some embodiments, the disease is Alzheimer's disease. In some embodiments, the disease is Huntington's disease. In some embodiments, the disease is Parkinson's disease. In some embodiments, the disease is amyotrophic lateral sclerosis. In some embodiments, the disease is a cancer. In some embodiments, the disease is a hematological malignancy. In some embodiments, the disease is leukemia. In some embodiments, the disease is acute myeloid leukemia. In some embodiments, the disease is acute lymphoblastic leukemia. In some embodiments, the disease is colon cancer. In some embodiments, the disease is pancreatic cancer. In some embodiments, the disease is a metabolic disease. In some embodiments, the disease is diabetes. In some embodiments, the disease is Type II diabetes. In some embodiments, the disease is obesity.

In another aspect, provided herein is a method of inhibiting the aberrantly high activity and/or production of a glycogen synthase kinase 3 (GSK3) in a subject in need thereof, cell, tissue, or biological sample, the method comprising administering to the subject or contacting the cell, tissue, or biological sample with an effective amount of: a compound useful in a provided method or a pharmaceutical composition provided herein.

In another aspect, provided herein is a method of inhibiting the aberrantly high activity and/or production of a glycogen synthase kinase 3 (GSK3) in a cell, tissue, or biological sample, the method comprising contacting the cell, tissue, or biological sample with an effective amount of: a compound useful in a provided method or a pharmaceutical composition provided herein, wherein the cell, tissue, or biological sample is in vitro.

In certain embodiments, the cell, tissue, or biological sample is in vitro. In certain embodiments, the cell, tissue, or biological sample is in vivo.

In certain embodiments, the subject is an animal. In certain embodiments, the subject is a human. In certain embodiments, the subject is a human aged 18 years and older. In some embodiments, the subject is a human aged<2 years. In some embodiments, the subject is a human aged 2-6 years, inclusive. In some embodiments, the subject is a human aged 6-18 years, inclusive. In some embodiments, the subject is a human aged 18-65 years, inclusive. In some embodiments, the subject is a human aged≥65 years. In certain embodiments, the subject is a non-human animal. In certain embodiments, the subject is a mammal. In certain embodiments, the subject is a non-human mammal. In some embodiments, the subject is a research animal.

### NUMBERED EMBODIMENTS

1. A compound, or a pharmaceutically acceptable salt, solvate, hydrate, polymorph, co-crystal, tautomer, stereoisomer, isotopically labeled compound, or prodrug thereof, wherein the compound is of Formula I:

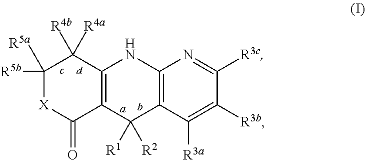

wherein:
R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R² is optionally substituted alkyl, optionally substituted alkenyl, or optionally substituted alkynyl;
or R¹ and R² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
each of R^{3a} and R^{3b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A}, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)OR^{A}, —OS(═O)₂SR^{A}, —OS(═O)₂N(R^{A})₂, —ON(R^{A}), —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃;
or R^{3a} and R^{3b} are taken together with their intervening atoms to form an optionally substituted, monocyclic, carbocyclic, heterocyclic, or heteroaryl ring, or an optionally substituted phenyl ring;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring.
R^{3c} is hydrogen;
X is

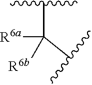

or —N(R⁶)—;
each of R^{4a}, R^{4b}, R^{5a}, R^{5b}, R^{6a}, and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
or R^{4a} and R^{4b} are taken together with their intervening atom to form an optionally substituted, monocyclic, carbocyclic or heterocyclic ring;
or R^{4b} and R^{5a} are taken together with their intervening atoms to form an optionally substituted, monocyclic, carbocyclic or heterocyclic ring;
or R^{5a} and R^{5b} are taken together with their intervening atom to form an optionally substituted, monocyclic, carbocyclic or heterocyclic ring;
or R^{5b} and R^{6a} are taken together with their intervening atoms to form an optionally substituted, monocyclic, carbocyclic or heterocyclic ring;
or R^{6a} and R^{6b} are taken together with their intervening atom to form an optionally substituted, monocyclic, carbocyclic or heterocyclic ring; and
R⁶ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.
2. The compound of embodiment 1, or a pharmaceutically acceptable salt, solvate, hydrate, polymorph, co-crystal, tautomer, stereoisomer, isotopically labeled compound, or prodrug thereof, wherein

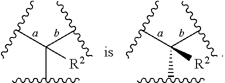

3. The compound of embodiment 1 or 2, or a pharmaceutically acceptable salt, solvate, hydrate, polymorph, co-crystal, tautomer, stereoisomer, isotopically labeled compound, or prodrug thereof, wherein R¹ is optionally substituted aryl.
4. The compound of embodiment 3, or a pharmaceutically acceptable salt, solvate, hydrate, polymorph, co-crystal, tautomer, stereoisomer, isotopically labeled compound, or prodrug thereof, wherein R¹ is optionally substituted phenyl.
5. The compound of embodiment 1 or 2, or a pharmaceutically acceptable salt, solvate, hydrate, polymorph, co-crystal, tautomer, stereoisomer, isotopically labeled compound, or prodrug thereof, wherein R¹ is

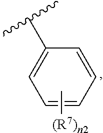

wherein:
   (i) each instance of R⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂ORD, —S(═O)₂SR^{D}, —S(═O)₂N(R^{D})₂, —S(═O)(═NR^{D})R^{D}, —S(═O)(═NR^{D})ORD, —S(═O)(═NR^{D})SR^{D}, —S(═O)(═NR^{D})N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —OS(═O)(═NR^{D})R^{D}, —OS(═O)(═NR^{D})OR^{D}, —OS(═O)(═NR^{D})SR^{D}, —OS(═O)(═NR^{D})N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SRD, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NRDS(═O)₂R^{D}, —NR^{D}S(═O)₂OR^{D}, —NRDS(═O)₂SR^{D}, —NR^{D}S(═O)₂N(R^{D})₂, —NR^{D}S(═O)(═NR^{D})R^{D}, —NR^{D}S(═O)(═NR^{D})OR^{D}, —NR^{D}S(═O)(═NR^{D})NR^{D}SR^{D}, —NR^{D}S(O)(═NR^{D})N(R^{D})₂, —Si(R^{D})₃, —Si(R^{D})₂OR^{D}, —Si(R^{D})(OR^{D}), —Si(OR^{D})₃, —OSi(R^{D})₃, —OSi(R^{D})₂OR^{D}, —OSi(R^{D})(OR^{D})₂, or —OSi(OR^{D})₃; or
   (ii) two instances of R⁷ on two adjacent carbon atoms are taken together with the two adjacent carbon atoms to form an optionally substituted, monocyclic, aryl, heteroaryl, carbocyclic, or heterocyclic ring, and the remaining instances of R⁷, if present, are as defined in (i);
   each instance of R^{D} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{D} attached to the same intervening atom are joined together with the intervening atom to form an optionally substituted, monocyclic, heterocyclic or heteroaryl ring, and
   n2 is 0, 1, 2, 3, 4, or 5.
6. The compound of embodiment 5, or a pharmaceutically acceptable salt, solvate, hydrate, polymorph, co-crystal, tautomer, stereoisomer, isotopically labeled compound, or prodrug thereof, wherein the compound is of Formula I-1:

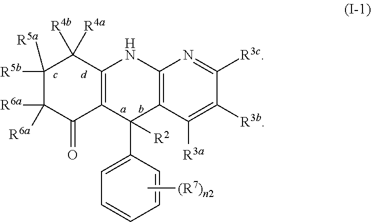

7. The compound of embodiment 5, or a pharmaceutically acceptable salt, solvate, hydrate, polymorph, co-crystal, tautomer, stereoisomer, isotopically labeled compound, or prodrug thereof, wherein the compound is of Formula I-2:

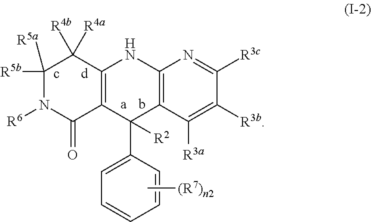

8. The compound of any one of embodiments 5-7, or a pharmaceutically acceptable salt, solvate, hydrate, polymorph, co-crystal, tautomer, stereoisomer, isotopically labeled compound, or prodrug thereof, wherein n2 is 0.
9. The compound of any one of embodiments 5-7, or a pharmaceutically acceptable salt, solvate, hydrate, polymorph, co-crystal, tautomer, stereoisomer, isotopically labeled compound, or prodrug thereof, wherein R¹ is

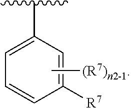

10. The compound of embodiment 5, or a pharmaceutically acceptable salt, solvate, hydrate, polymorph, co-crystal, tautomer, stereoisomer, isotopically labeled compound, or prodrug thereof, wherein the compound is of Formula I-3:

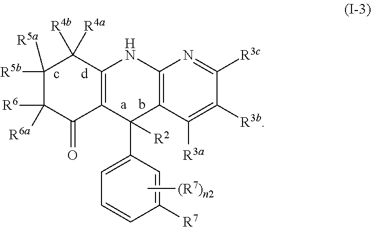

11. The compound of embodiment 5, or a pharmaceutically acceptable salt, solvate, hydrate, polymorph, co-crystal, tautomer, stereoisomer, isotopically labeled compound, or prodrug thereof, wherein the compound is of Formula I-4:

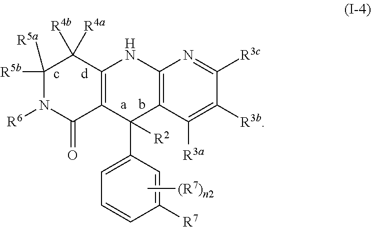

12. The compound of any one of embodiments 5-7 and 9-11, or a pharmaceutically acceptable salt, solvate, hydrate, polymorph, co-crystal, tautomer, stereoisomer, isotopically labeled compound, or prodrug thereof, wherein n2 is 1.
13. The compound of any one of embodiments 5-7 and 9-12, or a pharmaceutically acceptable salt, solvate, hydrate, polymorph, co-crystal, tautomer, stereoisomer, isotopically labeled compound, or prodrug thereof, wherein at least one instance of R⁷ is optionally substituted alkyl.
14. The compound of any one of embodiments 5-7 and 9-13, or a pharmaceutically acceptable salt, solvate, hydrate, polymorph, co-crystal, tautomer, stereoisomer, isotopically labeled compound, or prodrug thereof, wherein at least one instance of R⁷ is unsubstituted C₁₋₆ alkyl or C₁₋₆ alkyl substituted with one or more halogen, as valency permits.
15. The compound of any one of embodiments 5-7 and 9-14, or a pharmaceutically acceptable salt, solvate, hydrate, polymorph, co-crystal, tautomer, stereoisomer, isotopically labeled compound, or prodrug thereof, wherein at least one instance of R⁷ is optionally substituted carbocyclyl.
16. The compound of any one of embodiments 5-7 and 9-15, or a pharmaceutically acceptable salt, solvate, hydrate, polymorph, co-crystal, tautomer, stereoisomer, isotopically labeled compound, or prodrug thereof, wherein at least one instance of R⁷ is optionally substituted monocyclic C₃₋₇ carbocyclyl.
17. The compound of any one of embodiments 5-7 and 9-16, or a pharmaceutically acceptable salt, solvate, hydrate, polymorph, co-crystal, tautomer, stereoisomer, isotopically labeled compound, or prodrug thereof, wherein at least one instance of R⁷ is optionally substituted heteroaryl.
18. The compound of any one of embodiments 5-7 and 9-17, or a pharmaceutically acceptable salt, solvate, hydrate, polymorph, co-crystal, tautomer, stereoisomer, isotopically labeled compound, or prodrug thereof, wherein at least one instance of R⁷ is optionally substituted heteroaryl comprising one or more nitrogen atoms in the heteroaryl ring system, as valency permits.
19. The compound of any one of embodiments 5-7 and 9-18, or a pharmaceutically acceptable salt, solvate, hydrate, polymorph, co-crystal, tautomer, stereoisomer, isotopically labeled compound, or prodrug thereof, wherein at least one instance of R⁷ is optionally substituted, 5- or 6-membered, monocyclic heteroaryl.
20. The compound of any one of embodiments 5-7 and 9-19, or a pharmaceutically acceptable salt, solvate, hydrate, polymorph, co-crystal, tautomer, stereoisomer, isotopically labeled compound, or prodrug thereof, wherein at least one instance of R⁷ is optionally substituted pyridinyl, optionally substituted pyridazinyl, optionally substituted pyrazolyl, or optionally substituted imidazolyl.
20A. The compound of any one of embodiments 5-7 and 9-20, or a pharmaceutically acceptable salt, solvate, hydrate, polymorph, co-crystal, tautomer, stereoisomer, isotopically labeled compound, or prodrug thereof, wherein at least one instance of R⁷ is optionally substituted pyridinyl.
20B. The compound of any one of embodiments 5-7 and 9-20, or a pharmaceutically acceptable salt, solvate, hydrate, polymorph, co-crystal, tautomer, stereoisomer, isotopically labeled compound, or prodrug thereof, wherein at least one instance of R⁷ is optionally substituted 4-pyridinyl.
21. The compound of any one of embodiments 5-7 and 9-20B, or a pharmaceutically acceptable salt, solvate, hydrate, polymorph, co-crystal, tautomer, stereoisomer, isotopically labeled compound, or prodrug thereof, wherein at least one instance of R⁷ is optionally substituted, 5- or 6-membered, monocyclic heteroaryl fused to an optionally substituted, monocyclic, carbocyclic, heterocyclic, aryl, or heteroaryl ring.
22. The compound of any one of embodiments 5-7 and 9-21, or a pharmaceutically acceptable salt, solvate, hydrate, polymorph, co-crystal, tautomer, stereoisomer, isotopically labeled compound, or prodrug thereof, wherein at least one instance of R⁷ is optionally substituted, 5- or 6-membered, monocyclic heteroaryl fused to an optionally substituted, 4- to 7-membered, monocyclic carbocyclic ring.
23. The compound of any one of embodiments 5-7 and 9-22, or a pharmaceutically acceptable salt, solvate, hydrate, polymorph, co-crystal, tautomer, stereoisomer, isotopically labeled compound, or prodrug thereof, wherein at least one instance of R⁷ is —S(optionally substituted alkyl), —S(═O)(═NH)(optionally substituted alkyl), —S(═O)(═N(unsubstituted C₁₋₆ alkyl))(optionally substituted alkyl), —S(═O)₂(optionally substituted alkyl), or —OH.
24. The compound of any one of embodiments 5-7 and 9-23, or a pharmaceutically acceptable salt, solvate, hydrate, polymorph, co-crystal, tautomer, stereoisomer, isotopically labeled compound, or prodrug thereof, wherein at least one instance of R⁷ is —CH(CH₃)₂, —CF₃, unsubstituted cyclobutyl, —SCH₃, —SCF₃, —S(═O)(═NH)(CH₃), —S(═O)(═NCH₃)(CH₃), —S(═O)₂CH₃, —S(═O)₂CF₃, or —OH.
25. The compound of any one of embodiments 1-24, or a pharmaceutically acceptable salt, solvate, hydrate, polymorph, co-crystal, tautomer, stereoisomer, isotopically labeled compound, or prodrug thereof, wherein R² is optionally substituted alkyl.
26. The compound of embodiment 25, or a pharmaceutically acceptable salt, solvate, hydrate, polymorph, co-crystal, tautomer, stereoisomer, isotopically labeled compound, or prodrug thereof, wherein R² is unsubstituted C₁₋₄ alkyl or C₁₋₄ alkyl substituted with one or more fluorine, as valency permits.
27. The compound of embodiment 25, or a pharmaceutically acceptable salt, solvate, hydrate, polymorph, co-crystal, tautomer, stereoisomer, isotopically labeled compound, or prodrug thereof, wherein R² is —CH₃.
28. The compound of embodiment 25, or a pharmaceutically acceptable salt, solvate, hydrate, polymorph, co-crystal, tautomer, stereoisomer, isotopically labeled compound, or prodrug thereof, wherein R² is —C₂H₅.
29. The compound of embodiment 1, or a pharmaceutically acceptable salt, solvate, hydrate, polymorph, co-crystal, tautomer, stereoisomer, isotopically labeled compound, or prodrug thereof, wherein R¹ and R² are taken together with their intervening atom to form an optionally substituted, monocyclic, carbocyclic ring, which is optionally fused to an optionally substituted, aryl, heteroaryl, carbocyclic, or heterocyclic ring and/or optionally forms a spiro linkage with an optionally substituted, carbocyclic or heterocyclic ring.
30. The compound of embodiment 1, or a pharmaceutically acceptable salt, solvate, hydrate, polymorph, co-crystal, tautomer, stereoisomer, isotopically labeled compound, or prodrug thereof, wherein R¹ and R² are taken together with their intervening atom to form an optionally substituted, monocyclic, heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring.
31. The compound of embodiment 1, or a pharmaceutically acceptable salt, solvate, hydrate, polymorph, co-crystal, tautomer, stereoisomer, isotopically labeled compound, or prodrug thereof, wherein R¹ and R² are taken together with their intervening atom to form an optionally substituted, 5-membered, monocyclic, heterocyclic ring, which is fused to an optionally substituted phenyl ring.
32. The compound of embodiment 1, or a pharmaceutically acceptable salt, solvate, hydrate, polymorph, co-crystal, tautomer, stereoisomer, isotopically labeled compound, or prodrug thereof, wherein R¹ and R² are taken together with their intervening atom to form

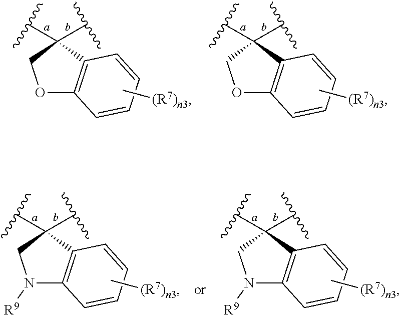

wherein
   (i) each instance of R⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —S(═O)(═NR^{D})R^{D}, —S(═O)(═NR^{D})OR^{D}, —S(═O)(═NR^{D})SR^{D}, —S(═O)(═NR^{D})N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)OR^{D}, —OS(═O)₂SR^{D}, —OS(═O)₂N(R^{D})₂, —OS(═O)(═NR^{D})R^{D}, —OS(═O)(═NR^{D})OR^{D}, —OS(═O)(═NR^{D})SR^{D}, —OS(═O)(═NR^{D})N(R^{D})₂. —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)OR^{D}, —NR^{D}S(═O)₂SR^{D}, —NR^{D}S(═O)₂N(R^{D})₂, —NR^{D}S(═O)(═NR^{D})R^{D}, —NR^{D}S(═O)(═NR^{D})OR^{D}, —NR^{D}S(═O)(═NR^{D})NR^{D}SR^{D}, —NR^{D}S(═O)(═NR^{D})N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃; or
   (ii) two instances of R⁷ on two adjacent carbon atoms are taken together with the two adjacent carbon atoms to form an optionally substituted, monocyclic, aryl, heteroaryl, carbocyclic, or heterocyclic ring, and the remaining instances of R⁷, if present, are as defined in (i);
   each instance of R^{D} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{D} attached to the same intervening atom are joined together with the intervening atom to form an optionally substituted, monocyclic, heterocyclic or heteroaryl ring;
   n3 is 0, 1, 2, 3, or 4; and
   R⁹ is hydrogen, optionally substituted alkyl, or a nitrogen protecting group.
33. The compound of any one of embodiments 1-32, or a pharmaceutically acceptable salt, solvate, hydrate, polymorph, co-crystal, tautomer, stereoisomer, isotopically labeled compound, or prodrug thereof, wherein R^{3a} is hydrogen.
34. The compound of any one of embodiments 1-32, or a pharmaceutically acceptable salt, solvate, hydrate, polymorph, co-crystal, tautomer, stereoisomer, isotopically labeled compound, or prodrug thereof, wherein R^{3a} is halogen, optionally substituted alkyl, optionally substituted alkenyl, optionally substituted carbocyclyl, or —CN.
35. The compound of any one of embodiments 1-32, or a pharmaceutically acceptable salt, solvate, hydrate, polymorph, co-crystal, tautomer, stereoisomer, isotopically labeled compound, or prodrug thereof, wherein R³ is halogen; unsubstituted C₁₋₆ alkyl; C₁₋₆ alkyl substituted with one or more halogen and/or one or more unsubstituted, monocyclic, 3- to 7-membered carbocyclyl, as valency permits; unsubstituted C₂₋₆ alkenyl; unsubstituted, monocyclic, 3- to 7-membered carbocyclyl; or —CN.
36. The compound of any one of embodiments 1-32, or a pharmaceutically acceptable salt, solvate, hydrate, polymorph, co-crystal, tautomer, stereoisomer, isotopically labeled compound, or prodrug thereof, wherein R³ is fluorine, chlorine, bromine, —CH₃, —C₂H₅, —CH(CH₃)₂, —CH₂C(CH₃)₃, —CF₃, —CH₂CF₃, —CH₂-(unsubstituted cyclopropyl), —CH═CH₂, unsubstituted cyclopropyl, unsubstituted cyclobutyl, or —CN.
37. The compound of any one of embodiments 1-32, or a pharmaceutically acceptable salt, solvate, hydrate, polymorph, co-crystal, tautomer, stereoisomer, isotopically labeled compound, or prodrug thereof, wherein R^{3a} is —CF₃.
37A. The compound of any one of embodiments 1-32, or a pharmaceutically acceptable salt, solvate, hydrate, polymorph, co-crystal, tautomer, stereoisomer, isotopically labeled compound, or prodrug thereof, wherein R^{3a} is —CN.
38. The compound of any one of embodiments 1-37A, or a pharmaceutically acceptable salt, solvate, hydrate, polymorph, co-crystal, tautomer, stereoisomer, isotopically labeled compound, or prodrug thereof, wherein R^{3b} is hydrogen.
39. The compound of any one of embodiments 1-37A, or a pharmaceutically acceptable salt, solvate, hydrate, polymorph, co-crystal, tautomer, stereoisomer, isotopically labeled compound, or prodrug thereof, wherein R^{3b} is halogen, optionally substituted alkyl, optionally substituted carbocyclyl, or —CN.
40. The compound of any one of embodiments 1-37A, or a pharmaceutically acceptable salt, solvate, hydrate, polymorph, co-crystal, tautomer, stereoisomer, isotopically labeled compound, or prodrug thereof, wherein R^{3b} is halogen; unsubstituted C₁₋₆ alkyl; C₁₋₆ alkyl substituted with one or more halogen, as valency permits; unsubstituted, monocyclic, 3- to 7-membered carbocyclyl; or —CN.
41. The compound of any one of embodiments 1-37A, or a pharmaceutically acceptable salt, solvate, hydrate, polymorph, co-crystal, tautomer, stereoisomer, isotopically labeled compound, or prodrug thereof, wherein R^{3b} is fluorine, chlorine, bromine, —CH₃, —C₂H₅, —CH(CH₃)₂, —CH₂C(CH₃)₃, —CF₃, —CH₂CF₃, —CH₂-(unsubstituted cyclopropyl), —CH═CH₂, unsubstituted cyclopropyl, unsubstituted cyclobutyl, or —CN.
42. The compound of any one of embodiments 1-37A, or a pharmaceutically acceptable salt, solvate, hydrate, polymorph, co-crystal, tautomer, stereoisomer, isotopically labeled compound, or prodrug thereof, wherein R^{3b} is fluorine or —CF₃.
43. The compound of any one of embodiments 1-42, or a pharmaceutically acceptable salt, solvate, hydrate, polymorph, co-crystal, tautomer, stereoisomer, isotopically labeled compound, or prodrug thereof, wherein at least one of R^{3a} and R^{3b} is fluorine or C₁₋₂ alkyl substituted with one or more fluorine.
44. The compound of any one of embodiments 1-32, or a pharmaceutically acceptable salt, solvate, hydrate, polymorph, co-crystal, tautomer, stereoisomer, isotopically labeled compound, or prodrug thereof, wherein R^{3a} and R^{3b} are taken together with their intervening atoms to form an optionally substituted, monocyclic, carbocyclic or heterocyclic ring.
45. The compound of embodiment 44, or a pharmaceutically acceptable salt, solvate, hydrate, polymorph, co-crystal, tautomer, stereoisomer, isotopically labeled compound, or prodrug thereof, wherein R^{3a} and R^{3b} are taken together with their intervening atoms to form an optionally substituted, monocyclic, 3- to 7-membered carbocyclic ring.
46. The compound of any one of embodiments 1-45, or a pharmaceutically acceptable salt, solvate, hydrate, polymorph, co-crystal, tautomer, stereoisomer, isotopically labeled compound, or prodrug thereof, wherein each of R^{4a} and R^{4b} is independently hydrogen, halogen, or optionally substituted alkyl.
47. The compound of any one of embodiments 1-46, or a pharmaceutically acceptable salt, solvate, hydrate, polymorph, co-crystal, tautomer, stereoisomer, isotopically labeled compound, or prodrug thereof, wherein R^{4a} is hydrogen.
48. The compound of any one of embodiments 1-47, or a pharmaceutically acceptable salt, solvate, hydrate, polymorph, co-crystal, tautomer, stereoisomer, isotopically labeled compound, or prodrug thereof, wherein R^{4b} is hydrogen.
49. The compound of any one of embodiments 1-47, or a pharmaceutically acceptable salt, solvate, hydrate, polymorph, co-crystal, tautomer, stereoisomer, isotopically labeled compound, or prodrug thereof, wherein R^{4b} is halogen.
50. The compound of embodiment 49, or a pharmaceutically acceptable salt, solvate, hydrate, polymorph, co-crystal, tautomer, stereoisomer, isotopically labeled compound, or prodrug thereof, wherein R^{4b} is fluorine.
51. The compound of any one of embodiments 1-45, or a pharmaceutically acceptable salt, solvate, hydrate, polymorph, co-crystal, tautomer, stereoisomer, isotopically labeled compound, or prodrug thereof, wherein R^{4a} and R^{4b} are taken together with their intervening atom to form an optionally substituted, monocyclic, C₃₋₇ carbocyclic ring.
52. The compound of any one of embodiments 1-45, or a pharmaceutically acceptable salt, solvate, hydrate, polymorph, co-crystal, tautomer, stereoisomer, isotopically labeled compound, or prodrug thereof, wherein R^{4a} and R^{4b} are taken together with their intervening atom to form an optionally substituted, cyclopropyl, cyclobutyl, cyclopentyl, or cyclohexyl ring.
53. The compound of any one of embodiments 1-50, or a pharmaceutically acceptable salt, solvate, hydrate, polymorph, co-crystal, tautomer, stereoisomer, isotopically labeled compound, or prodrug thereof, wherein

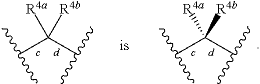

54. The compound of any one of embodiments 1-50, or a pharmaceutically acceptable salt, solvate, hydrate, polymorph, co-crystal, tautomer, stereoisomer, isotopically labeled compound, or prodrug thereof, wherein

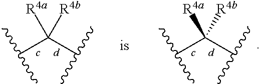

55. The compound of any one of embodiments 1-54, or a pharmaceutically acceptable salt, solvate, hydrate, polymorph, co-crystal, tautomer, stereoisomer, isotopically labeled compound, or prodrug thereof, wherein each of R^{5a} and R^{5b} is independently hydrogen, halogen, or optionally substituted alkyl.
56. The compound of any one of embodiments 1-55, or a pharmaceutically acceptable salt, solvate, hydrate, polymorph, co-crystal, tautomer, stereoisomer, isotopically labeled compound, or prodrug thereof, wherein R^{5a} is optionally substituted alkyl.
57. The compound of embodiment 56, or a pharmaceutically acceptable salt, solvate, hydrate, polymorph, co-crystal, tautomer, stereoisomer, isotopically labeled compound, or prodrug thereof, wherein R^{5a} is unsubstituted C₁₋₄ alkyl.
58. The compound of embodiment 56, or a pharmaceutically acceptable salt, solvate, hydrate, polymorph, co-crystal, tautomer, stereoisomer, isotopically labeled compound, or prodrug thereof, wherein R^{5a} is —CH₃.
59. The compound of any one of embodiments 1-58, or a pharmaceutically acceptable salt, solvate, hydrate, polymorph, co-crystal, tautomer, stereoisomer, isotopically labeled compound, or prodrug thereof, wherein R^{5b} is optionally substituted alkyl.
60. The compound of embodiment 59, or a pharmaceutically acceptable salt, solvate, hydrate, polymorph, co-crystal, tautomer, stereoisomer, isotopically labeled compound, or prodrug thereof, wherein R^{5b} is unsubstituted C₁₋₄ alkyl.
61. The compound of embodiment 59, or a pharmaceutically acceptable salt, solvate, hydrate, polymorph, co-crystal, tautomer, stereoisomer, isotopically labeled compound, or prodrug thereof, wherein R^{5b} is —CH₃.
62. The compound of any one of embodiments 1-54, or a pharmaceutically acceptable salt, solvate, hydrate, polymorph, co-crystal, tautomer, stereoisomer, isotopically labeled compound, or prodrug thereof, wherein R^{5a} and R^{5b} are taken together with their intervening atom to form an optionally substituted, monocyclic, carbocyclic ring.
63. The compound of embodiment 62, or a pharmaceutically acceptable salt, solvate, hydrate, polymorph, co-crystal, tautomer, stereoisomer, isotopically labeled compound, or prodrug thereof, wherein R^{5a} and R^{5b} are taken together with their intervening atom to form an unsubstituted, monocyclic, 3- to 7-membered carbocyclic ring.
64. The compound of embodiment 62, or a pharmaceutically acceptable salt, solvate, hydrate, polymorph, co-crystal, tautomer, stereoisomer, isotopically labeled compound, or prodrug thereof, wherein R^{5a} and R^{5b} are taken together with their intervening atom to form an optionally substituted cyclobutyl ring.
65. The compound of any one of embodiments 1-5, 8, 9, and 12-64, or a pharmaceutically acceptable salt, solvate, hydrate, polymorph, co-crystal, tautomer, stereoisomer, isotopically labeled compound, or prodrug thereof, wherein X is

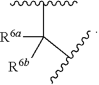

66. The compound of any one of embodiments 1-6, 8-10, and 12-65, or a pharmaceutically acceptable salt, solvate, hydrate, polymorph, co-crystal, tautomer, stereoisomer, isotopically labeled compound, or prodrug thereof, wherein each of R^{6a} and R^{6b} is independently hydrogen, halogen, or optionally substituted alkyl.
67. The compound of any one of embodiments 1-6, 8-10, and 12-66, or a pharmaceutically acceptable salt, solvate, hydrate, polymorph, co-crystal, tautomer, stereoisomer, isotopically labeled compound, or prodrug thereof, wherein R^{6a} is hydrogen.
68. The compound of any one of embodiments 1-6, 8-10, and 12-67, or a pharmaceutically acceptable salt, solvate, hydrate, polymorph, co-crystal, tautomer, stereoisomer, isotopically labeled compound, or prodrug thereof, wherein R^{6b} is hydrogen.
69. The compound of any one of embodiments 1-5, 8, 9, and 12-64, or a pharmaceutically acceptable salt, solvate, hydrate, polymorph, co-crystal, tautomer, stereoisomer, isotopically labeled compound, or prodrug thereof, wherein X is —N(R⁶)—.
70. The compound of any one of embodiments 1-5, 7-9, 11-64, and 69, or a pharmaceutically acceptable salt, solvate, hydrate, polymorph, co-crystal, tautomer, stereoisomer, isotopically labeled compound, or prodrug thereof, wherein R⁶ is hydrogen, optionally substituted alkyl, or a nitrogen protecting group.
71. The compound of embodiment 70, or a pharmaceutically acceptable salt, solvate, hydrate, polymorph, co-crystal, tautomer, stereoisomer, isotopically labeled compound, or prodrug thereof, wherein R⁶ is hydrogen.
72. The compound ofany one of embodiments 1-71 or a pharmaceutically acceptable salt, solvate, hydrate, polymorph, co-crystal, tautomer, stereoisomer, isotopically labeled compound, or prodrug thereof, wherein the molecular weight of the compound is not greater than 500 g/mol.
73. The compound of embodiment 1, or a pharmaceutically acceptable salt, solvate, hydrate, polymorph, co-crystal, tautomer, stereoisomer, isotopically labeled compound, or prodrug thereof, wherein the compound is of any one of the formulae:

| No. | Formula |
|---|---|
| 101R | 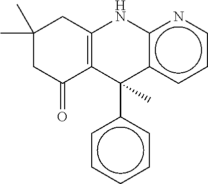 |
| 101S | 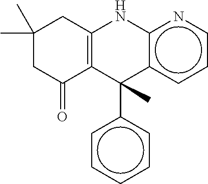 |
| 102R | 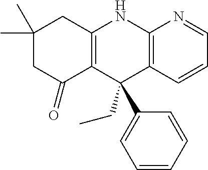 [SMILES: CC[C@]1(c2ccccc2)C2=C(CC(C)(C)CC2=O)Nc2ncccc21] |
| 102S | 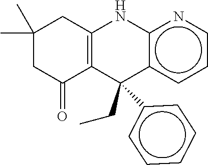 |
| 103R | 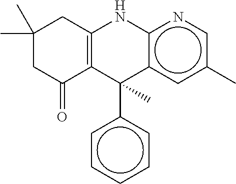 |
| 103S | 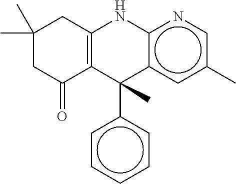 |
| 104R | 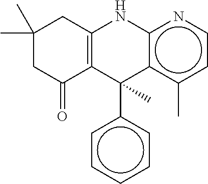 |
| 104S | 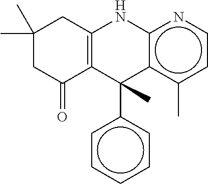 |
| 105R | 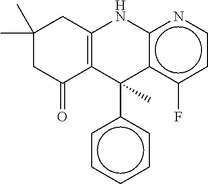 |
| 105S | 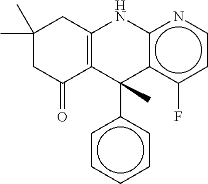 |
| 107R | 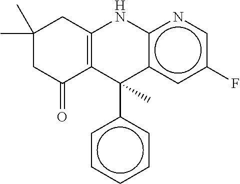 |
| 107S | 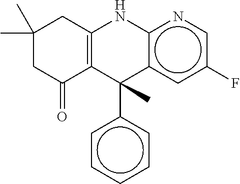 |
| 108R | 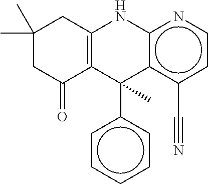 |
| 108S | 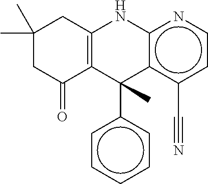 |
| 109R | 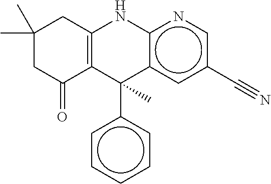 |
| 109S | 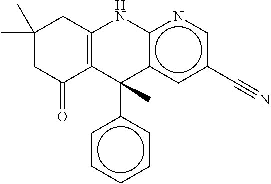 |
| 110R | 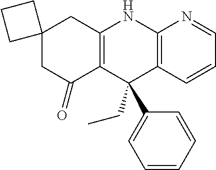 [SMILES: CC[C@]1(c2ccccc2)C2=C(CC3(CCC3)CC2=O)Nc2ncccc21] |
| 110S | 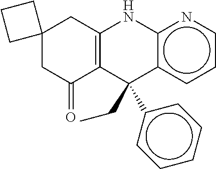 |
| 111R | 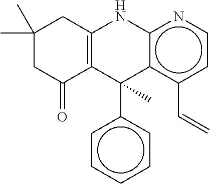 |
| 111S | 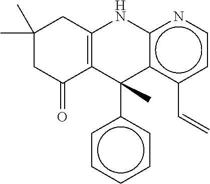 |
| 112R | 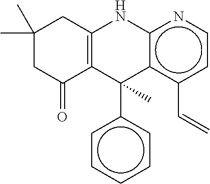 |
| 112S | 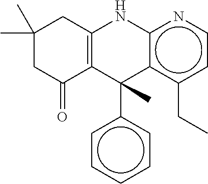 |
| 113R | 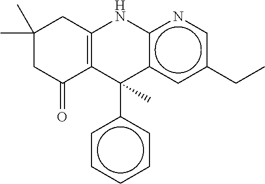 |
| 113S | 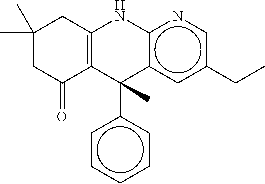 |
| 114R | 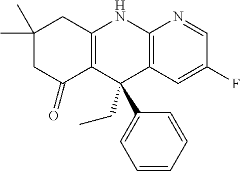 [SMILES: CC[C@]1(c2ccccc2)C2=C(CC(C)(C)CC2=O)Nc2ncc(F)cc21] |
| 114S | 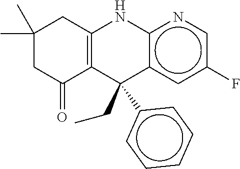 |
| 115R | 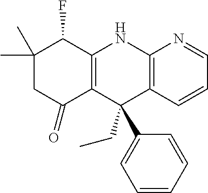 [SMILES: CC[C@]1(c2ccccc2)C2=C(Nc3ncccc31)[C@@H](F)C(C)(C)CC2=O] |
| 115S | 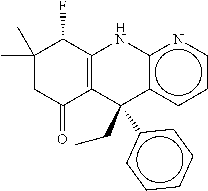 |
| 116R | 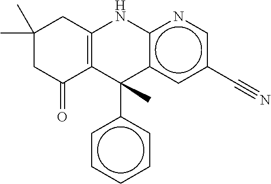 |
| 116S | 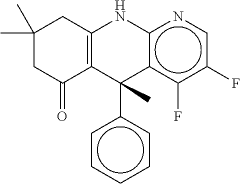 |
| 117R | 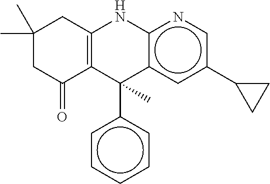 |
| 117S | 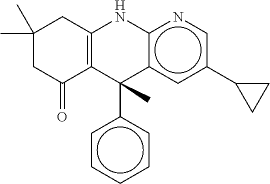 |
| 118R | 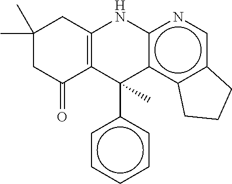 |
| 118S | 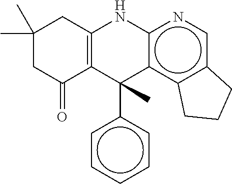 |
| 119R | 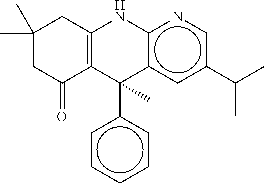 |
| 119S | 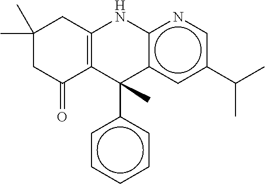 |
| 120R | 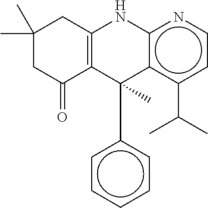 |
| 120S | 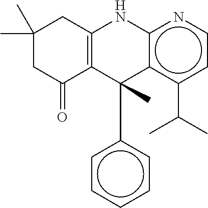 |
| 121R | 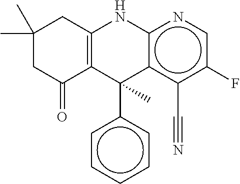 |
| 121S | 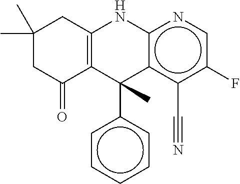 |
| 122R | 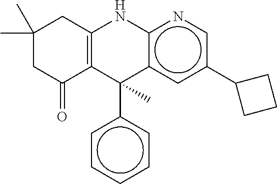 |
| 122S | 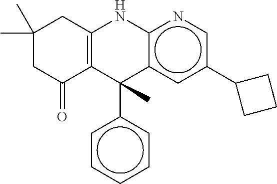 |
| 123R | 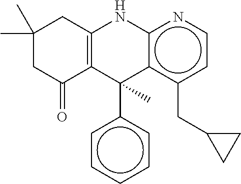 |
| 123S | 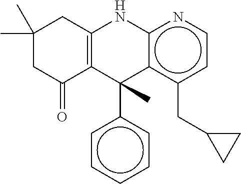 |
| 124R | 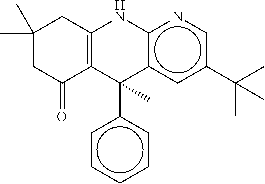 |
| 124S | 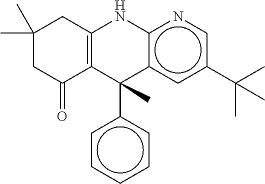 |
| 125R | 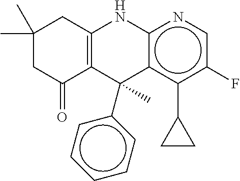 |
| 125S | 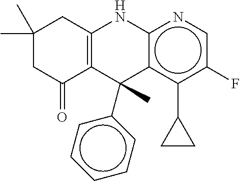 |
| 126R | 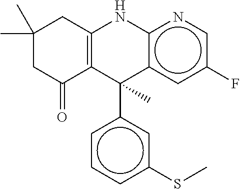 |
| 126S | 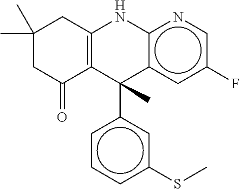 |
| 127R | 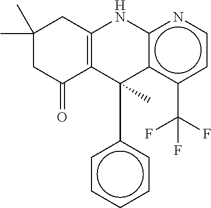 |
| 127S | 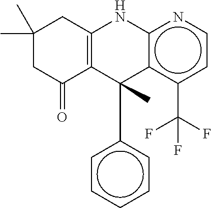 |
| 128R | 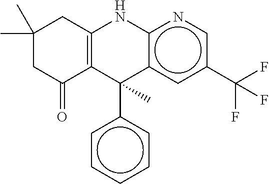 |
| 128S | 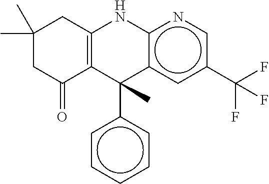 |
| 129R | 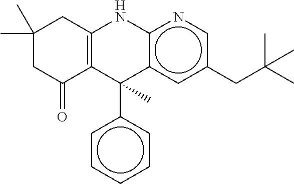 |
| 129S | 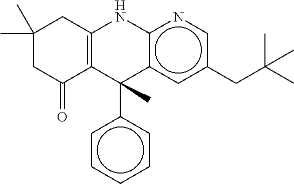 |
| 130R | 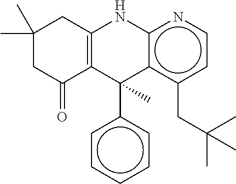 |
| 130S | 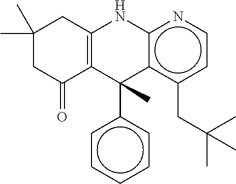 |
| 131R | 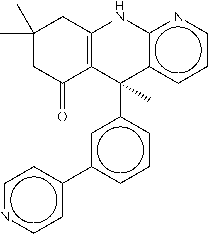 |
| 131S | 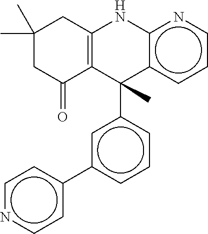 |
| 132R | 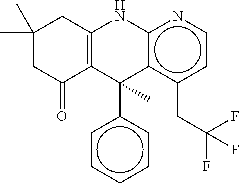 |
| 132S | 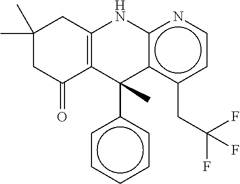 |
| 133R | 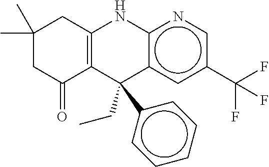 |
| 133S | 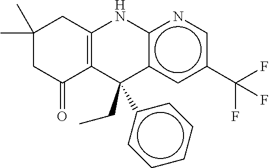 |
| 134R | 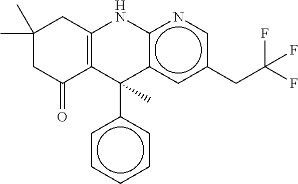 |
| 134S | 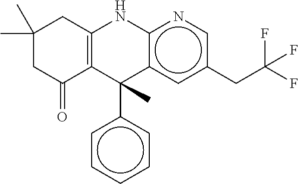 |
| 135R | 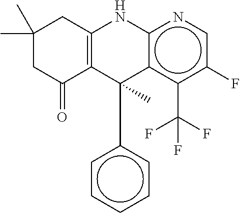 |
| 135S | 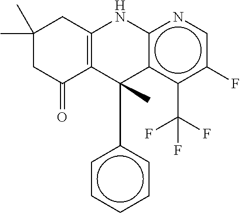 |
| 136R | 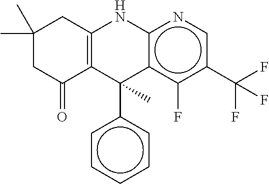 |
| 136S | 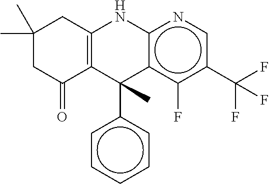 |
| 137R | 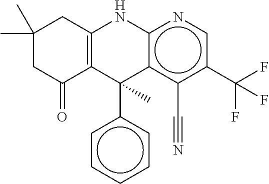 |
| 137S | 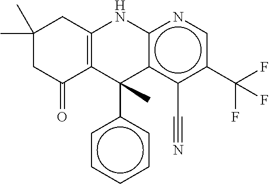 |
| 138R | 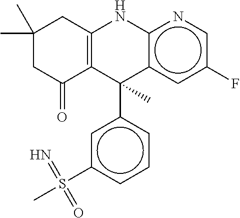 |
| 138S | 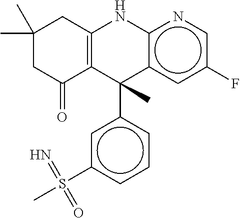 |
| 139R | 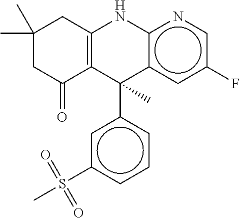 |
| 139S | 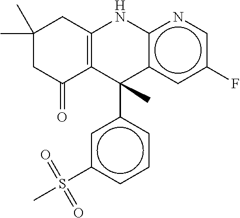 |
| 140R | 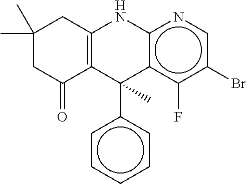 |
| 140S | 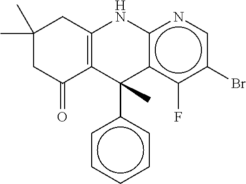 |
| 141R | 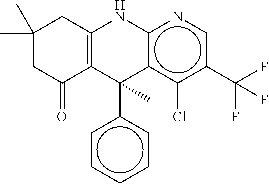 |
| 141S | 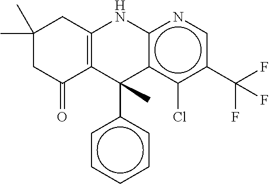 |
| 142R | 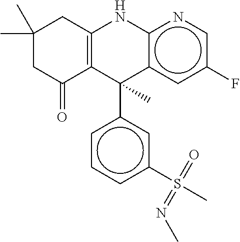 |
| 142S | 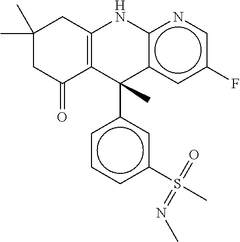 |
| 143R | 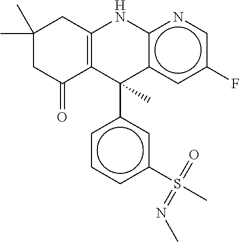 |
| 143S | 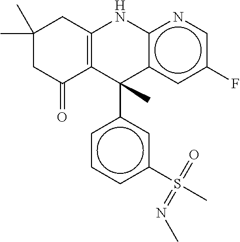 |
| 144R | 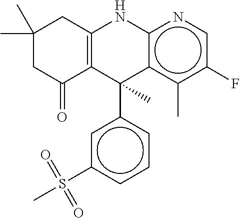 |
| 144S | 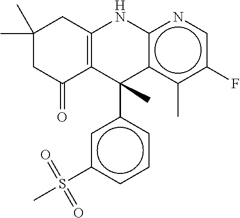 |
| 145R | 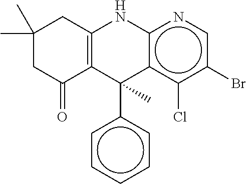 |
| 145S | 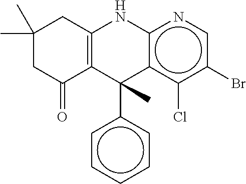 |
| 146R | 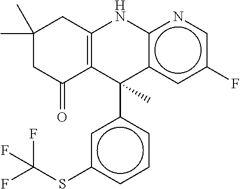 |
| 146S | 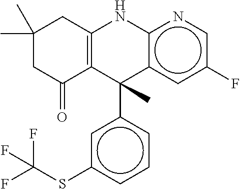 |
| 147R | 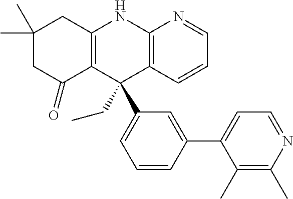 |
| 147S | 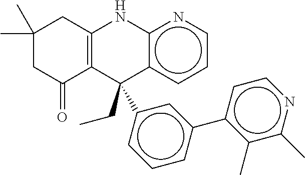 |
| 148R | 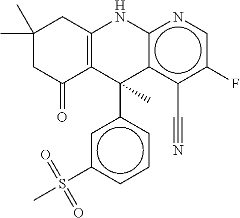 |
| 148S | 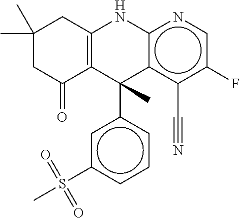 |
| 149R | 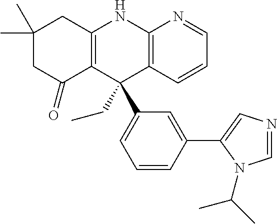 [SMILES: CC[C@]1(c2cccc(-c3cncn3C(C)C)c2)C2=C(CC(C)(C)CC2=O)Nc2ncccc21] |
| 149S | 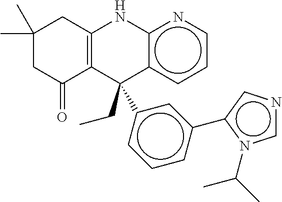 |
| 150R | 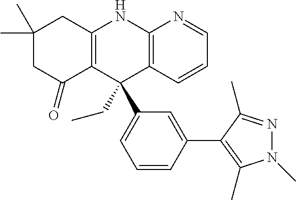 |
| 150S | 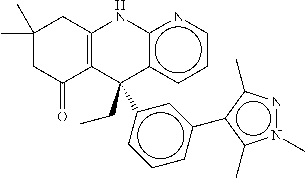 |
| 151R | 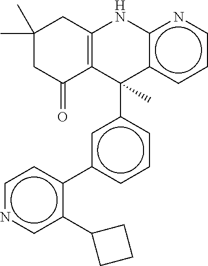 |
| 151S | 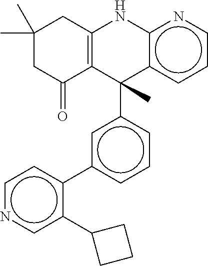 |
| 152R | 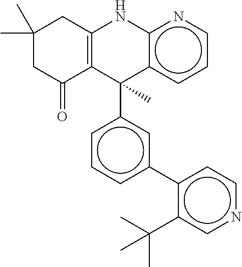 |
| 152S | 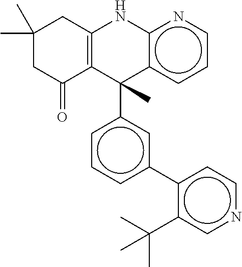 |
| 153R | 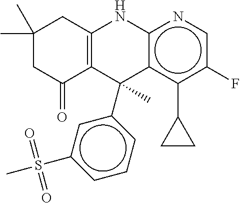 |
| 153S | 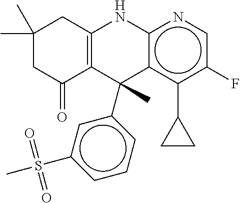 |
| 154R | 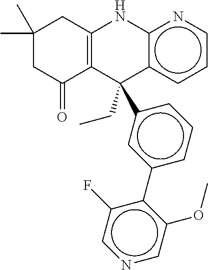 |
| 154S | 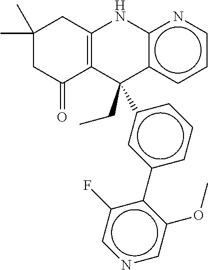 |
| 155R | 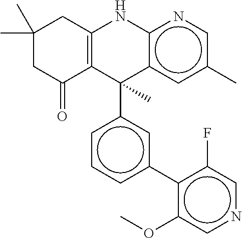 |
| 155S | 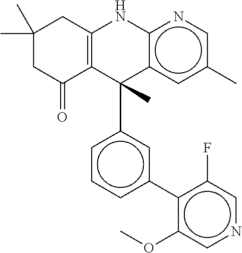 |
| 156R | 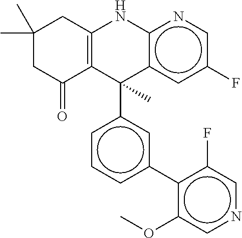 |
| 156S | 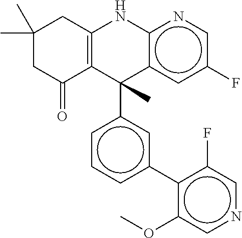 |
| 157R | 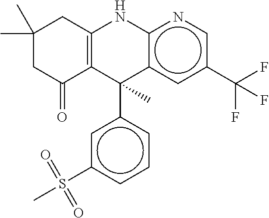 |
| 157S | 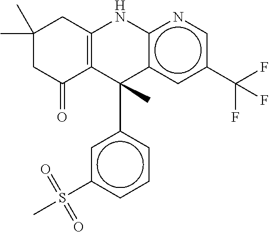 |
| 158R | 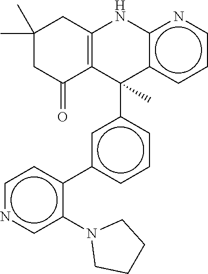 |
| 158S | 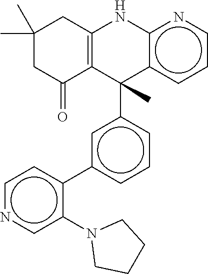 |
| 159R | 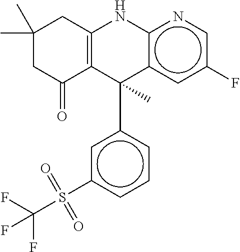 |
| 159S | 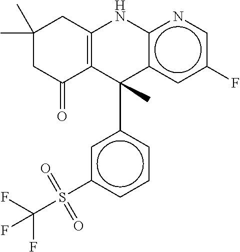 |
| 160R | 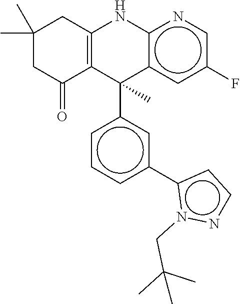 |
| 160S | 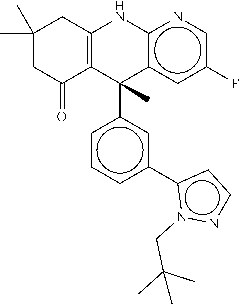 |
| 161R | 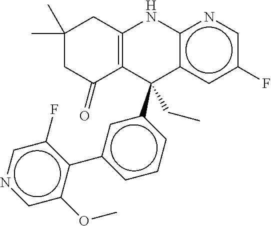 |
| 161S | 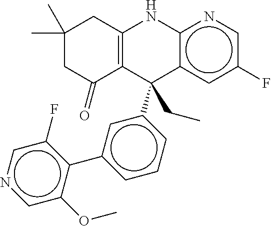 |
| 162R | 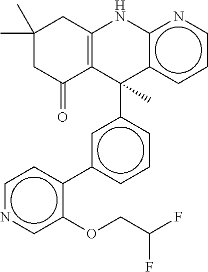 |
| 162S | 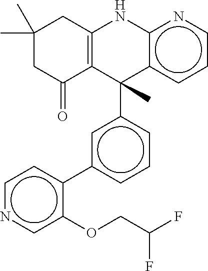 |
| 163R | 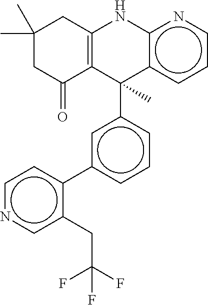 |
| 163S | 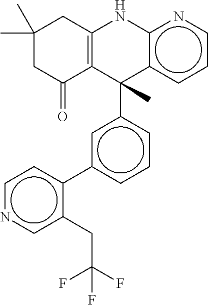 |
| 164R | 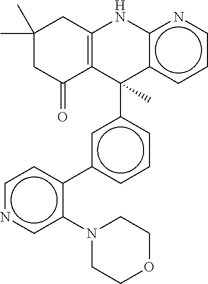 |
| 164S | 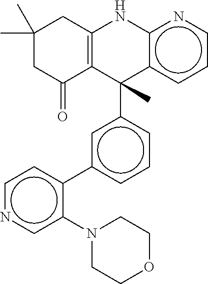 |
| 165R | 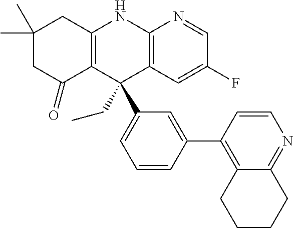 [SMILES: CC[C@]1(c2cccc(-c3ccnc4c3CCCC4)c2)C2=C(CC(C)(C)CC2=O)Nc2ncc(F)cc21] |
| 165S | 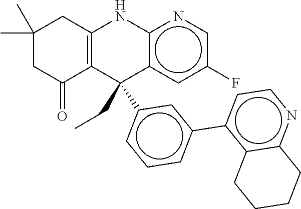 |
| 166R | 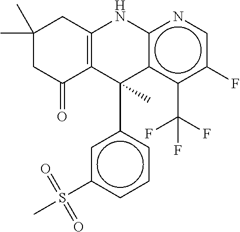 |
| 166S | 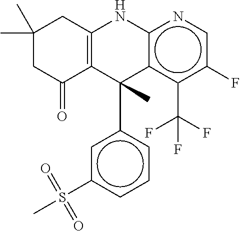 |
| 167R | 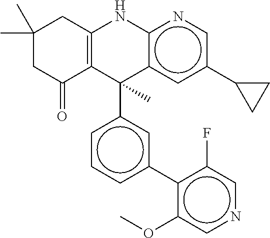 |
| 167S | 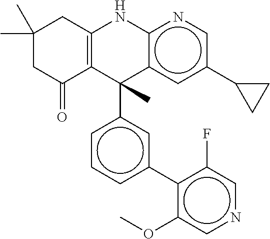 |
| 168R | 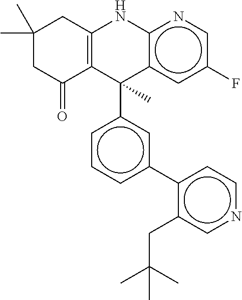 |
| 168S | 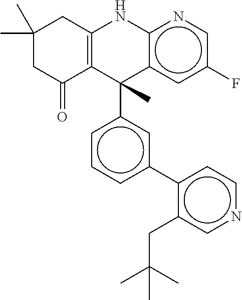 |
| 169R | 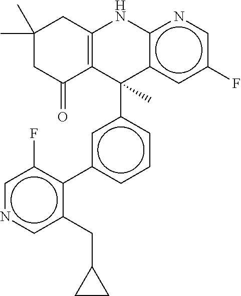 |
| 169S | 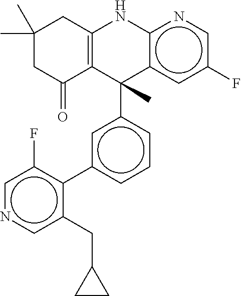 |
| 170R | 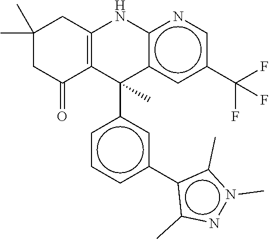 |
| 170S | 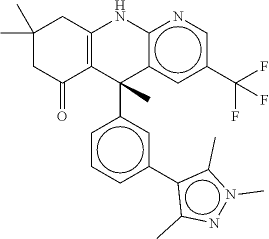 |
| 171R | 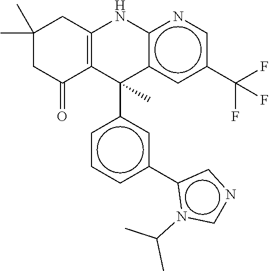 |
| 171S | 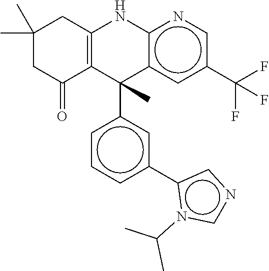 |
| 172R | 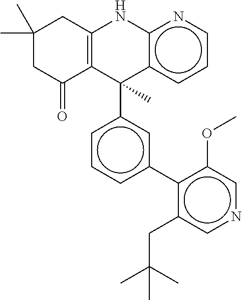 |
| 172S | 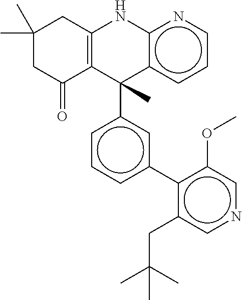 |
| 173R | 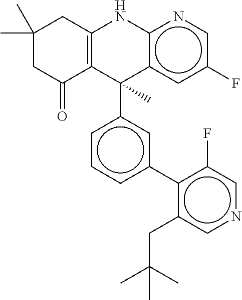 |
| 173S | 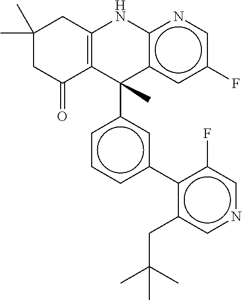 |
| 174R | 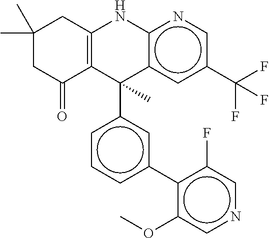 |
| 174S | 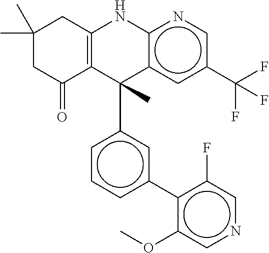 |
| 175R | 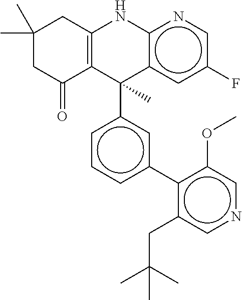 |
| 175S | 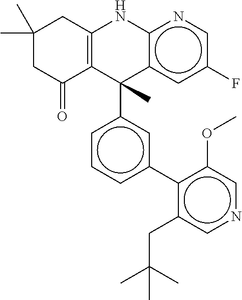 |
| 176R | 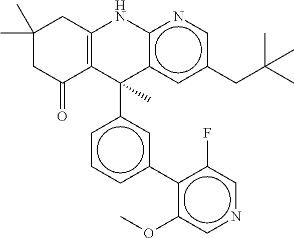 |
| 176S | 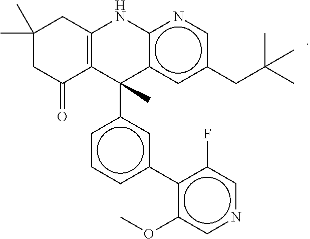 |

74. The compound of embodiment 1, or a pharmaceutically acceptable salt, solvate, hydrate, polymorph, co-crystal, tautomer, stereoisomer, isotopically labeled compound, or prodrug thereof, wherein the compound is of any one of the formulae:

| No. | Formula |
|---|---|
| 501R | 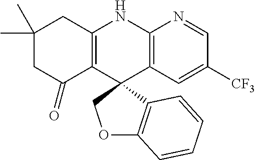 [SMILES: CC1(C)CC(=O)C2=C(C1)Nc1ncc(C(F)(F)F)cc1[C@@]21COc2ccccc21] |
| 501S | 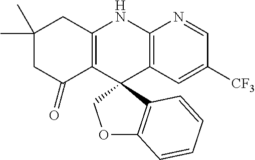 [SMILES: CC1(C)CC(=O)C2=C(C1)Nc1ncc(C(F)(F)F)cc1[C@]21COc2ccccc21] |
| 502R | 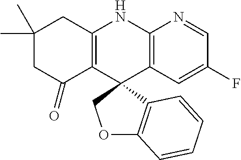 [SMILES: CC1(C)CC(=O)C2=C(C1)Nc1ncc(F)cc1[C@@]21COc2ccccc21] |
| 502S | 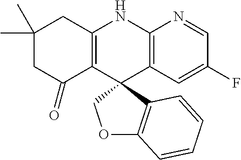 [SMILES: CC1(C)CC(=O)C2=C(C1)Nc1ncc(F)cc1[C@]21COc2ccccc21] |
| 503R | 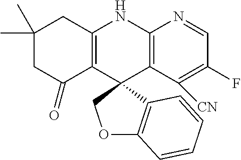 [SMILES: CC1(C)CC(=O)C2=C(C1)Nc1ncc(F)c(C#N)c1[C@@]21COc2ccccc21] |
| 503S | 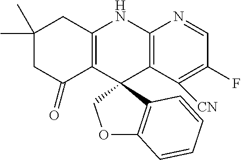 [SMILES: CC1(C)CC(=O)C2=C(C1)Nc1ncc(F)c(C#N)c1[C@]21COc2ccccc21] |
| 504R | 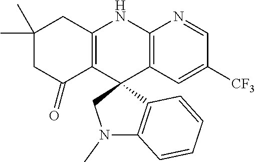 [SMILES: CN1C[C@]2(C3=C(CC(C)(C)CC3=O)Nc3ncc(C(F)(F)F)cc32)c2ccccc21] |
| 504S | 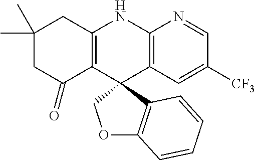 [SMILES: CC1(C)CC(=O)C2=C(C1)Nc1ncc(C(F)(F)F)cc1[C@]21COc2ccccc21] |
| 505R | 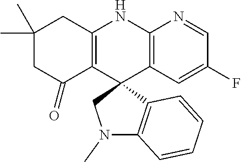 [SMILES: CN1C[C@]2(C3=C(CC(C)(C)CC3=O)Nc3ncc(F)cc32)c2ccccc21] |
| 505S | 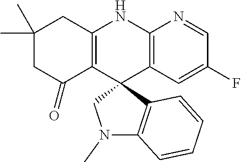 [SMILES: CN1C[C@@]2(C3=C(CC(C)(C)CC3=O)Nc3ncc(F)cc32)c2ccccc21] |
| 506R | 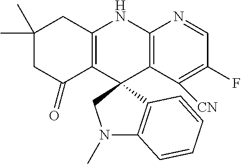 [SMILES: CN1C[C@]2(C3=C(CC(C)(C)CC3=O)Nc3ncc(F)c(C#N)c32)c2ccccc21] |
| 506S | 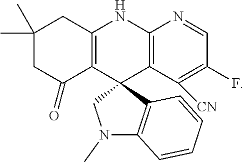 [SMILES: CN1C[C@@]2(C3=C(CC(C)(C)CC3=O)Nc3ncc(F)c(C#N)c32)c2ccccc21] |

74A. The compound of embodiment 1, or a pharmaceutically acceptable salt, solvate, hydrate, polymorph, co-crystal, tautomer, stereoisomer, isotopically labeled compound, or prodrug thereof, wherein the corn und is of any one of the formulae:

| No. | Formula |
|---|---|
| 601R | 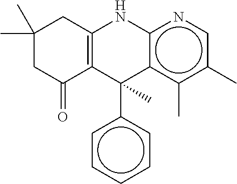 |
| 601S | 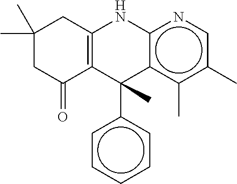 |
| 602R | 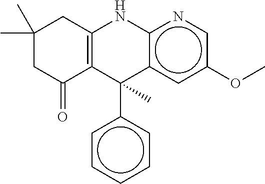 |
| 602S | 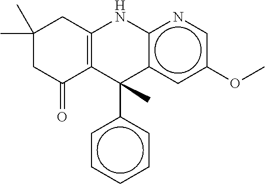 |
| 603R | 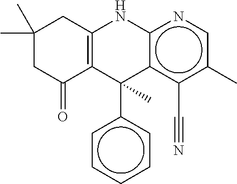 |
| 603S | 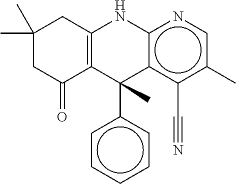 |
| 604R | 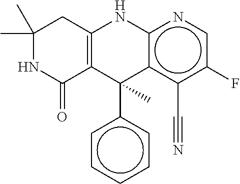 |
| 604S | 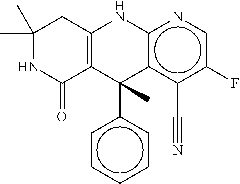 |
| 605R | 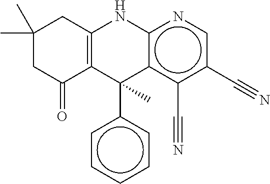 |
| 605S | 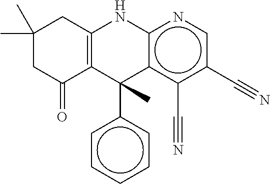 |
| 606R | 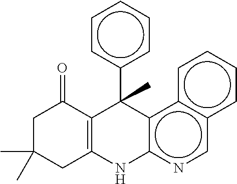 |
| 606S | 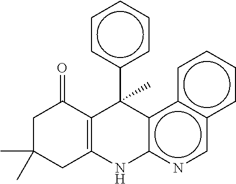 |
| 607R | 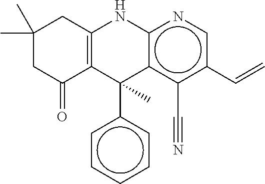 |
| 607S | 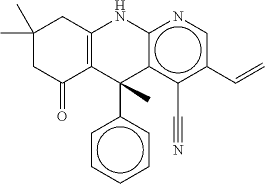 |
| 608R | 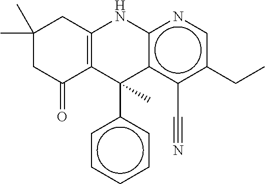 |
| 608S | 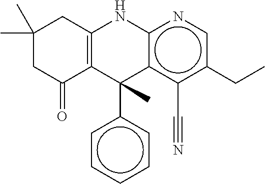 |
| 609R | 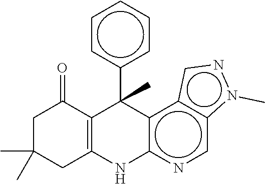 |
| 609S | 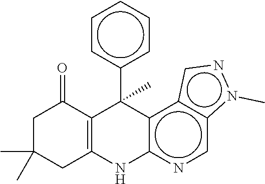 |
| 610R | 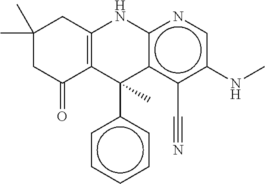 |
| 610S | 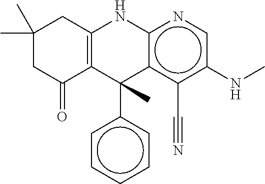 |
| 611R | 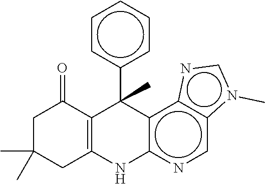 |
| 611S | 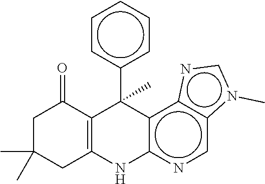 |
| 612R | 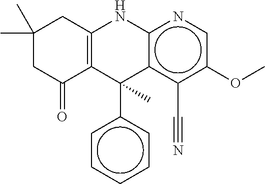 |
| 612S | 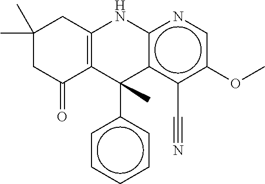 |
| 613R | 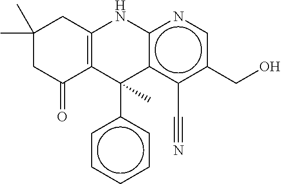 |
| 613S | 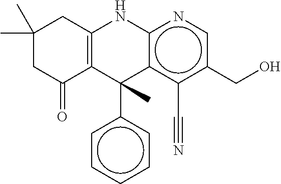 |
| 614R | 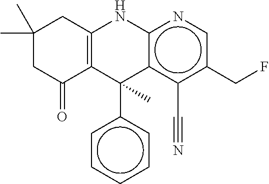 |
| 614S | 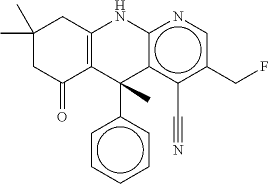 |
| 615R | 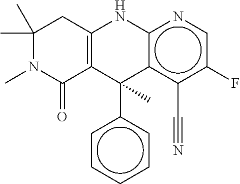 |
| 615S | 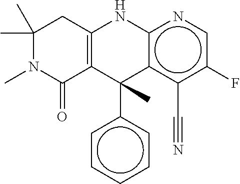 |
| 616RR | 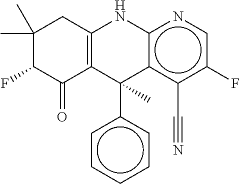 |
| 616SR | 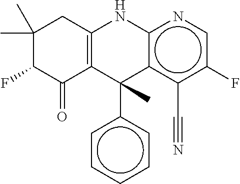 |
| 616RS | 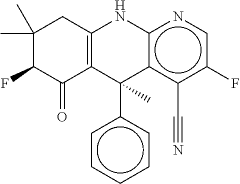 |
| 616SS | 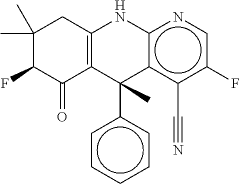 |
| 617RR | 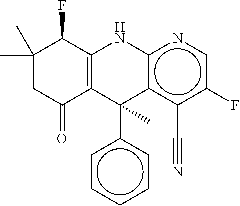 |
| 617SR | 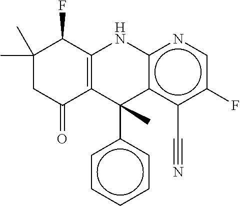 |
| 617RS | 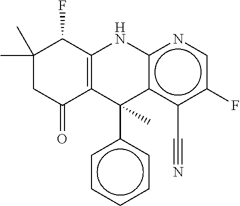 |
| 617SS | 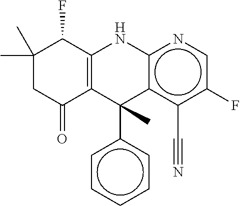 |
| 618R | 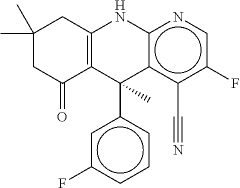 |
| 618S | 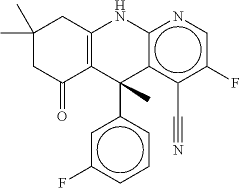 |
| 619R | 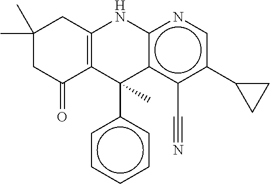 |
| 619S | 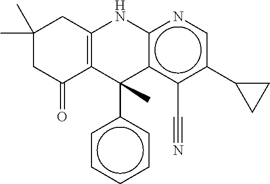 |
| 620R | 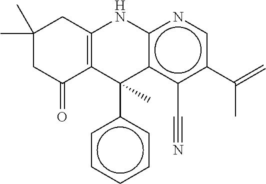 |
| 620S | 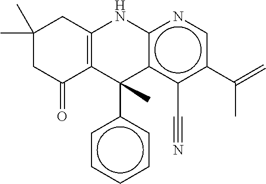 |
| 621R | 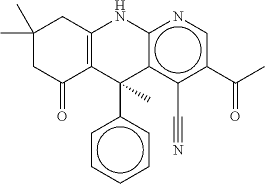 |
| 621S | 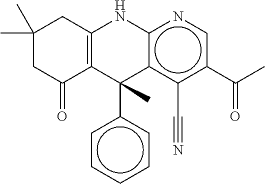 |
| 622R | 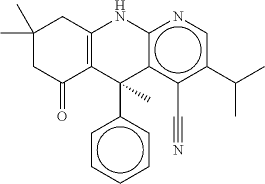 |
| 622S | 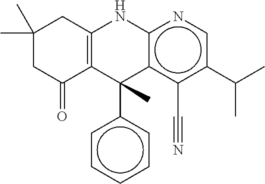 |
| 623R | 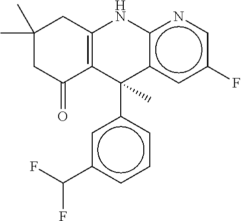 |
| 623S | 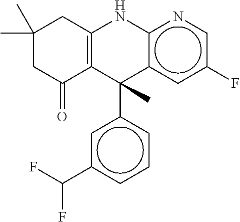 |
| 624R | 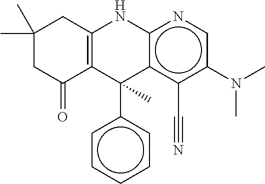 |
| 624S | 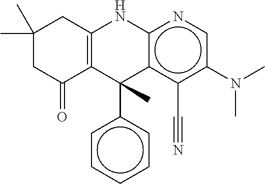 |
| 625R | 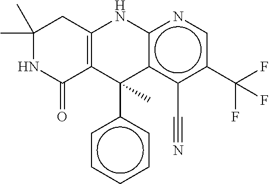 |
| 625S | 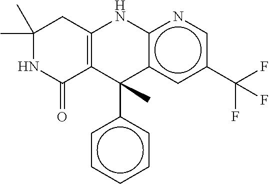 |
| 626R | 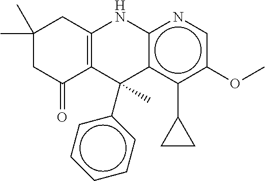 |
| 626S | 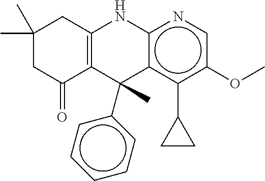 |
| 627R | 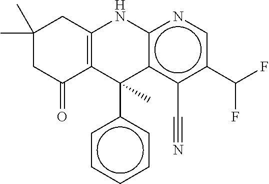 |
| 627S | 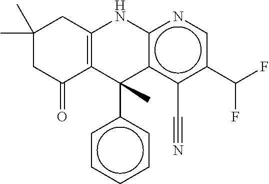 |
| 628R | 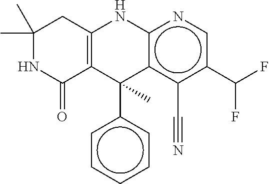 |
| 628S | 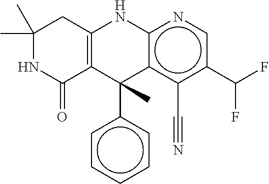 |
| 629R | 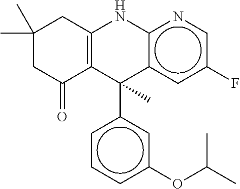 |
| 629S | 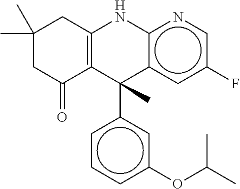 |
| 630R | 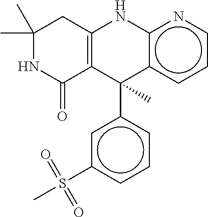 |
| 630S | 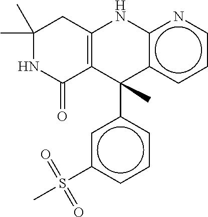 |
| 631R | 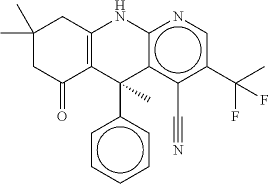 |
| 631S | 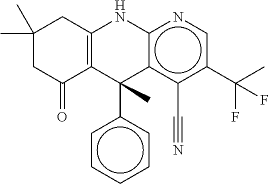 |
| 632R | 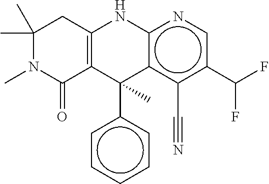 |
| 632S | 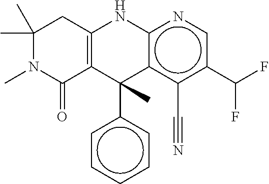 |
| 633RR | 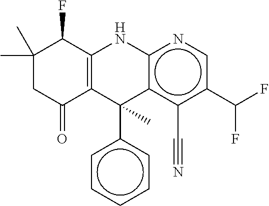 |
| 633SR | 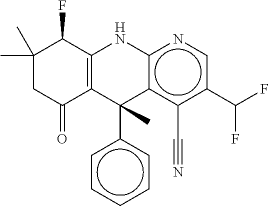 |
| 633RS | 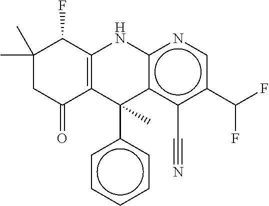 |
| 633SS | 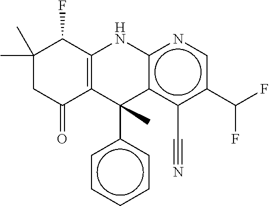 |
| 634R | 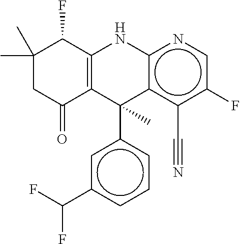 |
| 634S | 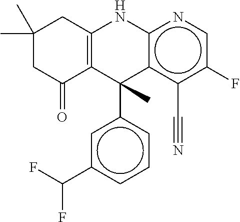 |
| 635R | 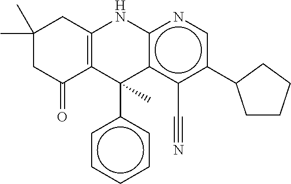 |
| 635S | 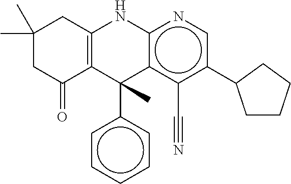 |
| 636R | 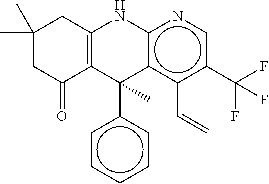 |
| 636S | 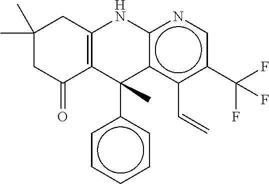 |
| 637R | 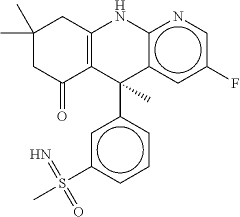 |
| 637S | 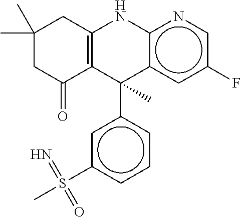 |
| 638R | 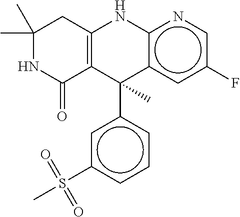 |
| 638S | 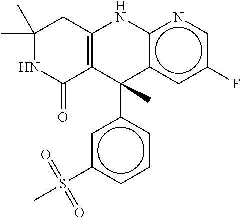 |
| 639R | 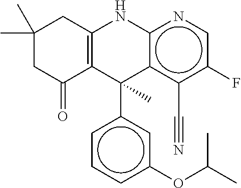 |
| 639S | 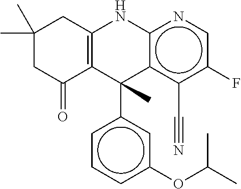 |
| 640R | 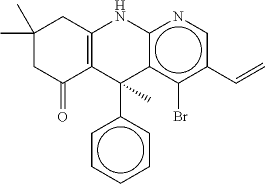 |
| 640S | 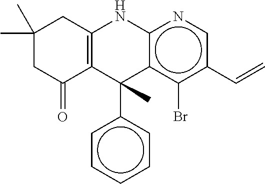 |
| 641R | 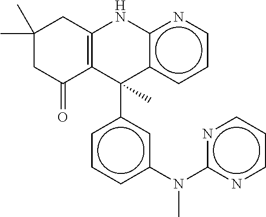 |
| 641S | 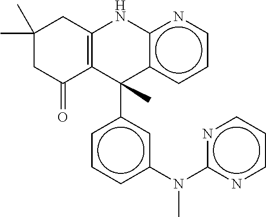 |
| 642R | 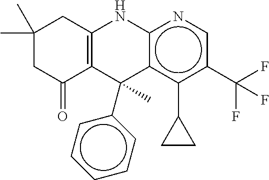 |
| 642S | 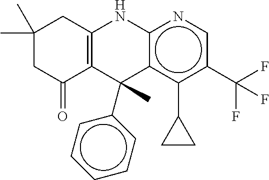 |
| 643R | 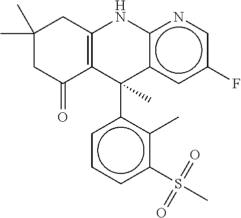 |
| 643S | 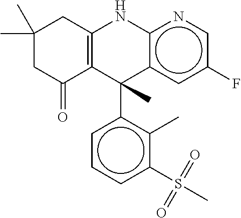 |
| 644R | 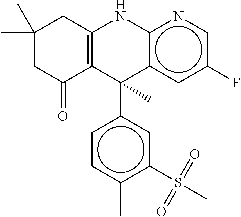 |
| 644S | 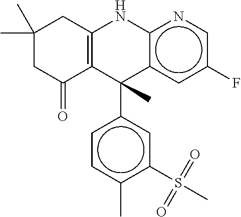 |
| 645R | 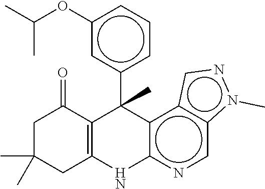 |
| 645S | 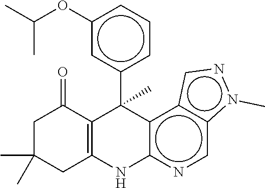 |
| 646R | 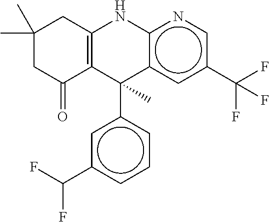 |
| 646S | 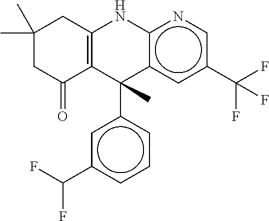 |
| 647R | 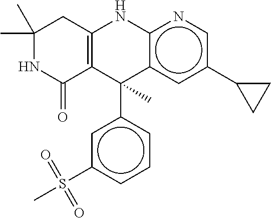 |
| 647S | 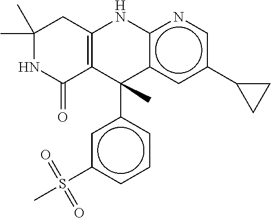 |
| 648R | 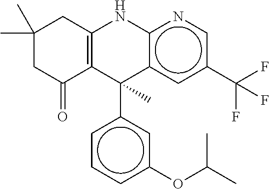 |
| 648S | 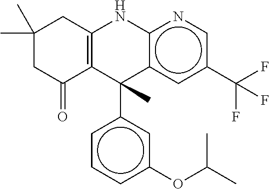 |
| 649R | 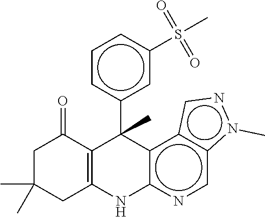 |
| 649S | 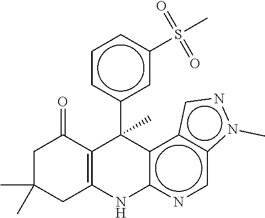 |
| 650R | 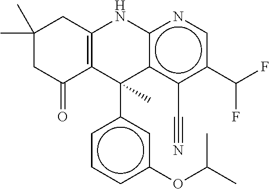 |
| 650S | 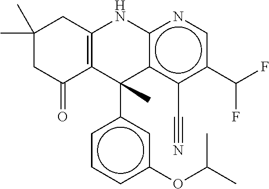 |
| 651R | 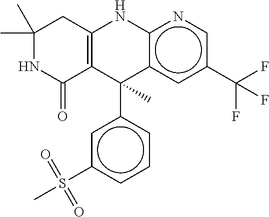 |
| 651S | 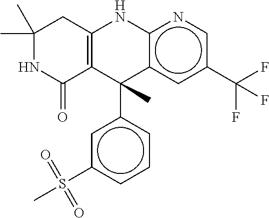 |
| 652R | 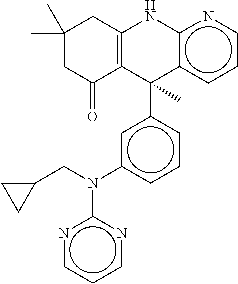 |
| 652S | 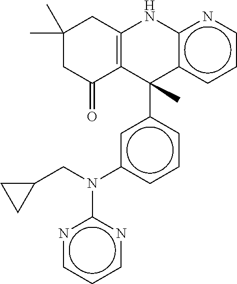 |
| 653R | 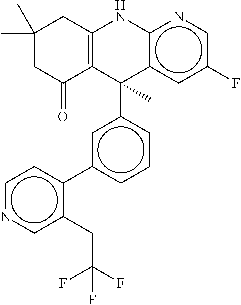 |
| 653S | 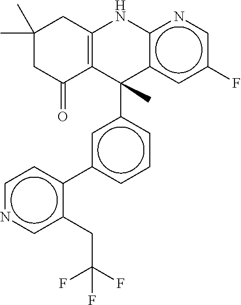 |
| 654RR | 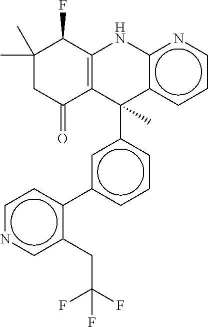 |
| 654SR | 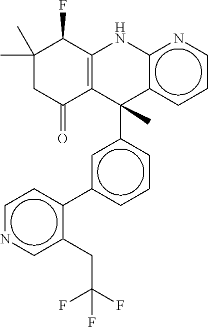 |
| 654RS | 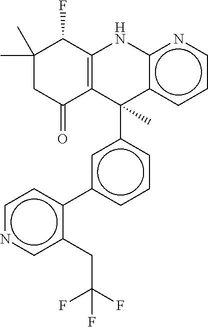 |
| 654SS | 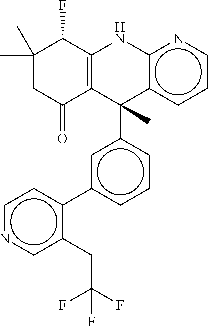 |
| 655RS | 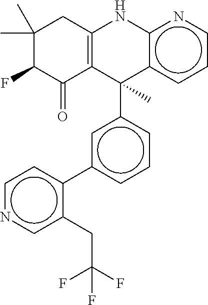 |
| 655SS | 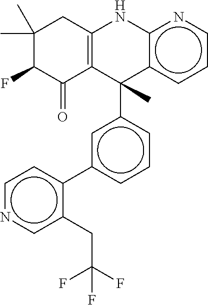 |
| 655RR | 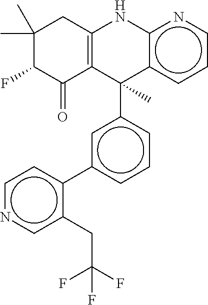 |
| 655SR | 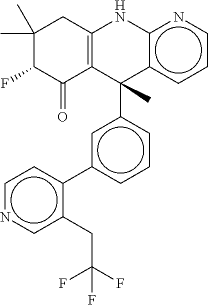 |
| 656R | 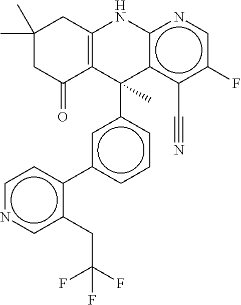 |
| 656S | 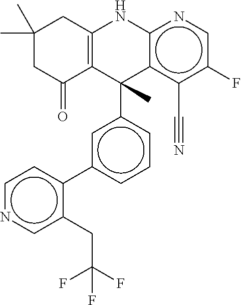 |
| 657R | 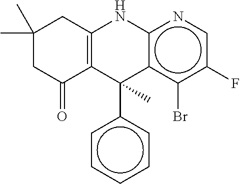 |
| 657S | 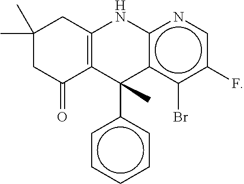 |

75. The compound of embodiment 1, or a pharmaceutically acceptable salt, solvate, hydrate, polymorph, co-crystal, tautomer, stereoisomer, isotopically labeled compound, or prodrug thereof, wherein the compound is of any one of the formulae:

| No. | Formula |
|---|---|
| 1002R | 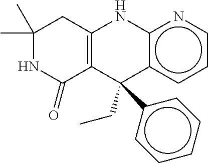 |
| 1002S | 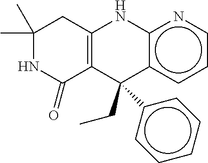 |
| 1004R | 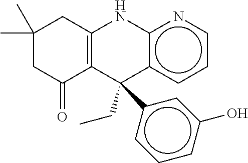 |
| 1004S | 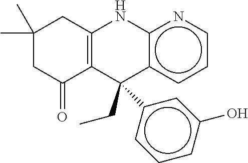 |
| 1006RR | 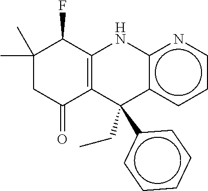 |
| 1006SR | 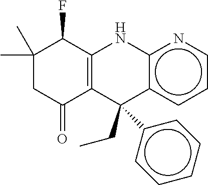 |
| 1006RS | 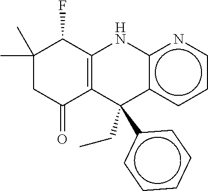 |
| 1006SS | 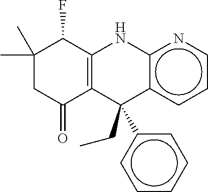 |
| 1007R | 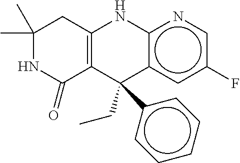 |
| 1007S | 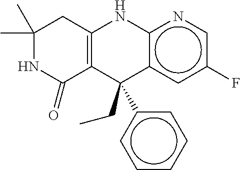 |
| 1008R | 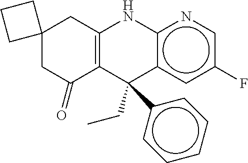 |
| 1008S | 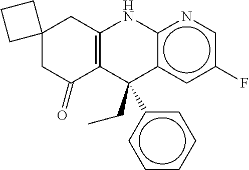 |
| 1009RR | 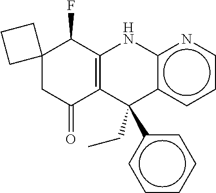 |
| 1009SR | 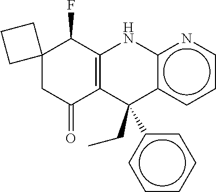 |
| 1009RS | 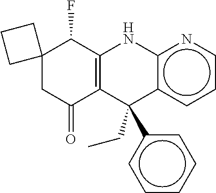 |
| 1009SS | 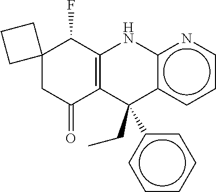 |
| 1010R | 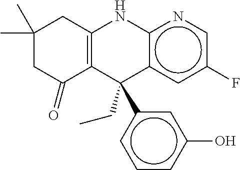 |
| 1010S | 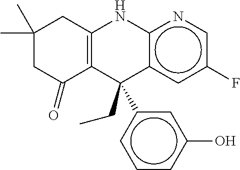 |
| 1011R | 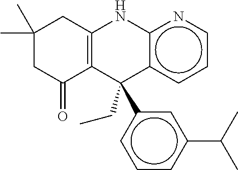 |
| 1011S | 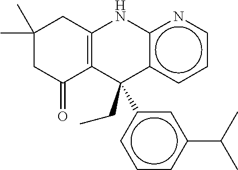 |
| 1012R | 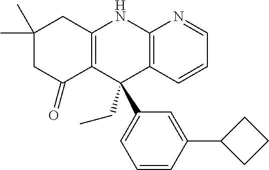 [SMILES: CC[C@]1(c2cccc(C3CCC3)c2)C2=C(CC(C)(C)CC2=O)Nc2ncccc21] |
| 1012S | 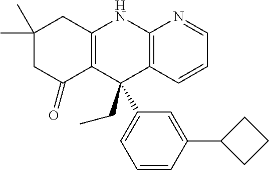 [SMILES: CC[C@@]1(c2cccc(C3CCC3)c2)C2=C(CC(C)(C)CC2=O)Nc2ncccc21] |
| 1014R | 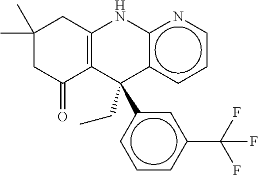 |
| 1014S | 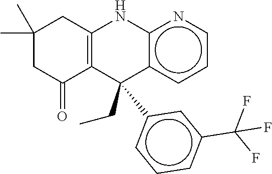 |
| 1015R | 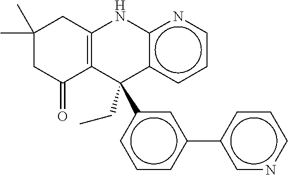 |
| 1015S | 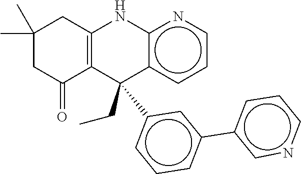 |
| 1016R | 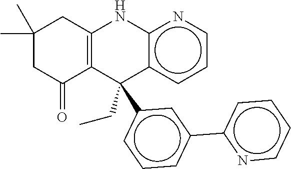 |
| 1016S | 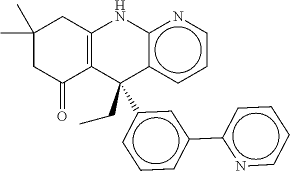 |
| 1017R | 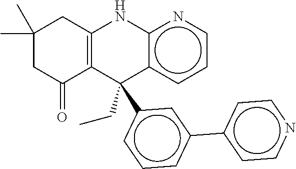 |
| 1017S | 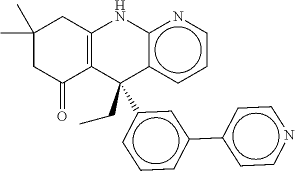 |
| 1018R | 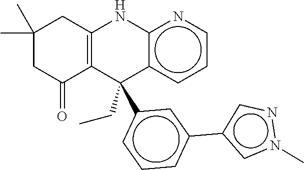 |
| 1018S | 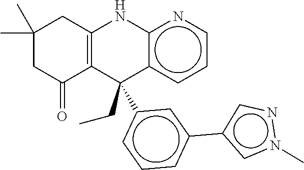 |
| 1019R | 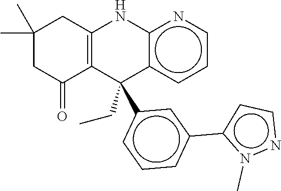 |
| 1019S | 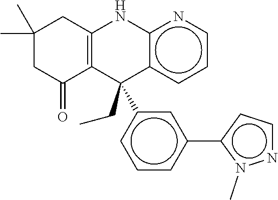 |
| 1020R | 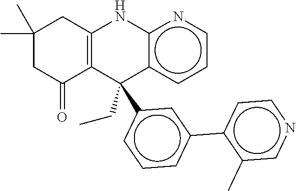 |
| 1020S | 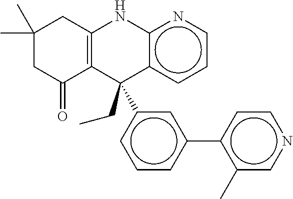 |
| 1021R | 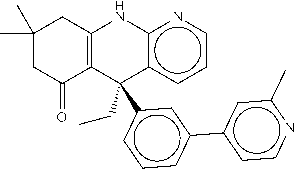 |
| 1021S | 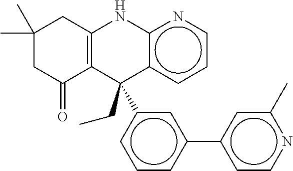 |
| 1022R | 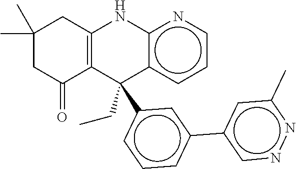 |
| 1022S | 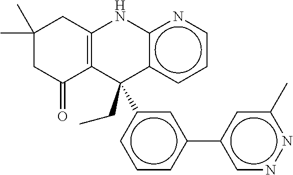 |
| 1023R | 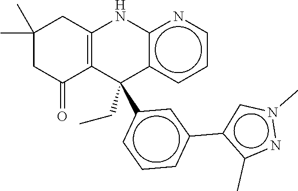 |
| 1023S | 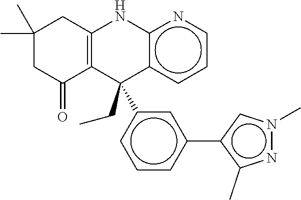 |
| 1024R | 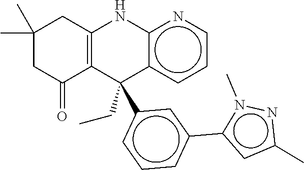 |
| 1024S | 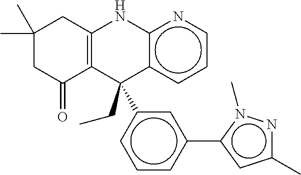 |
| 1025R | 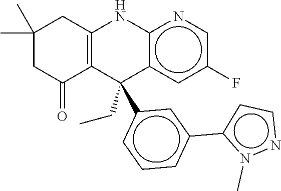 |
| 1025S | 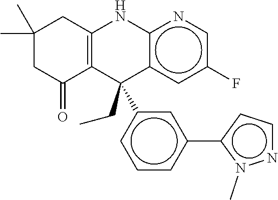 |
| 1027R | 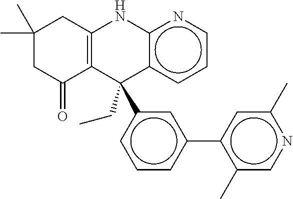 |
| 1027S | 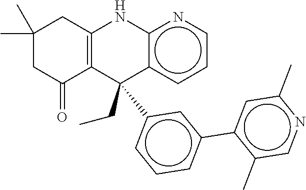 |
| 1028R | 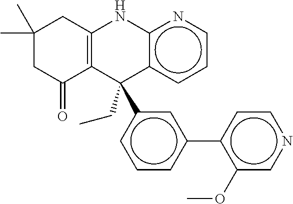 |
| 1028S | 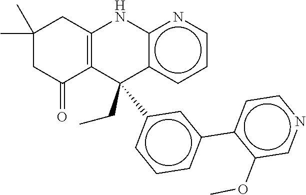 |
| 1031R | 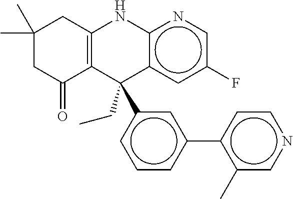 |
| 1031S | 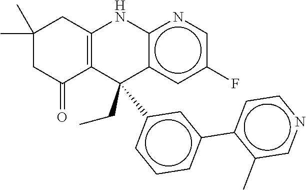 |
| 1032R | 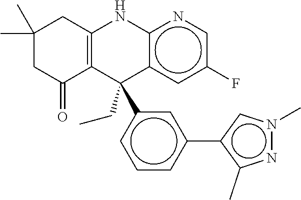 |
| 1032S | 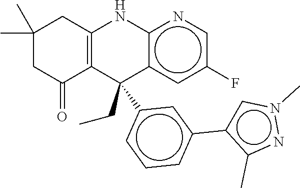 |
| 1033R | 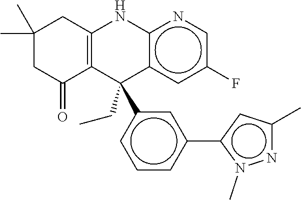 |
| 1033S | 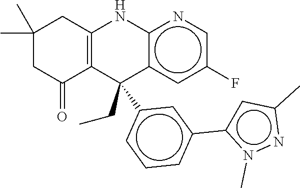 |
| 1034R | 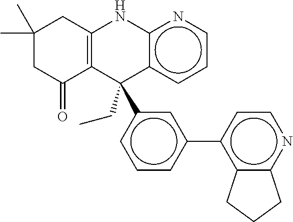 |
| 1034S | 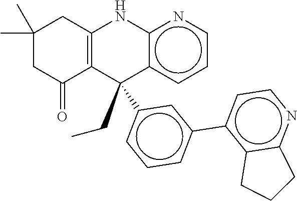 |
| 1035R | 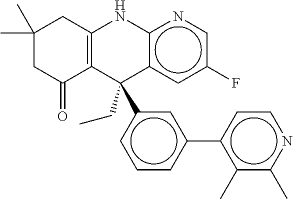 |
| 1035S | 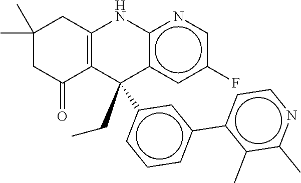 |
| 1037R | 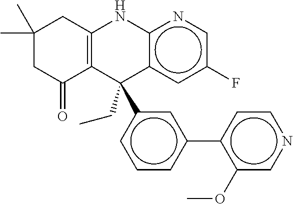 |
| 1037S | 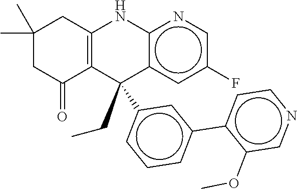 |
| 1038R | 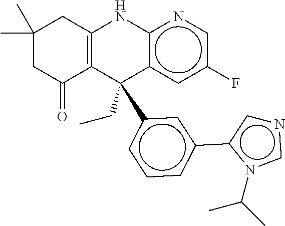 |
| 1038S | 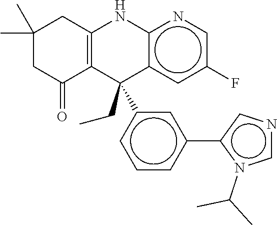 |
| 1039R | 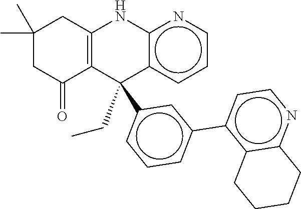 |
| 1039S | 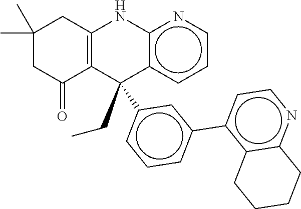 |

76. The compound of any one of embodiments 1-75, or a pharmaceutically acceptable salt thereof.
77. A pharmaceutical composition comprising:
   the compound of any one of embodiments 1-75, or a pharmaceutically acceptable salt, solvate, hydrate, polymorph, co-crystal, tautomer, stereoisomer, isotopically labeled compound, or prodrug thereof; and
   optionally a pharmaceutically acceptable excipient.
78. The pharmaceutical composition of embodiment 77 further comprising an additional pharmaceutical agent.
79. A kit comprising:
   the compound of any one of embodiments 1-75, or a pharmaceutically acceptable salt, solvate, hydrate, polymorph, co-crystal, tautomer, stereoisomer, isotopically labeled compound, or prodrug thereof, or the pharmaceutical composition of embodiment 77 or 78; and
   instructions for using the compound, or pharmaceutically acceptable salt, solvate, hydrate, polymorph, co-crystal, tautomer, stereoisomer, isotopically labeled compound, or prodrug thereof, or pharmaceutical composition.
80. A method of treating a disease in a subject in need thereof, the method comprising administering to the subject an effective amount of the compound of any one of embodiments 1-75, or a pharmaceutically acceptable salt, solvate, hydrate, polymorph, co-crystal, tautomer, stereoisomer, isotopically labeled compound, or prodrug thereof, or the pharmaceutical composition of embodiment 77 or 78.
81. A method of preventing a disease in a subject in need thereof, the method comprising administering to the subject an effective amount of the compound of any one of embodiments 1-75, or a pharmaceutically acceptable salt, solvate, hydrate, polymorph, co-crystal, tautomer, stereoisomer, isotopically labeled compound, or prodrug thereof, or the pharmaceutical composition of embodiment 77 or 78.
82. The method of embodiment 80 or 81, wherein the disease is associated with aberrantly high activity and/or production of a glycogen synthase kinase 3 (GSK3).
83. The method of embodiment 82, wherein the effective amount is further effective in inhibiting the aberrantly high activity and/or production of the GSK3.
84. The method of any one of embodiments 80-83, wherein the disease is a central nervous system disease.
85. The method of any one of embodiments 80-83, wherein the disease is a mental or behavioral disease.
86. The method of embodiment 85, wherein the disease is fragile X syndrome.
87. The method of embodiment 85, wherein the disease is autism.
88. The method of embodiment 85, wherein the disease is schizophrenia.
89. The method of embodiment 85, wherein the disease is bipolar disorder.
90. The method of embodiment 85, wherein the disease is attention deficit hyperactivity disorder.
91. The method of any one of embodiments 80-83, wherein the disease is a neurological disease.
92. The method of embodiment 91, wherein the disease is seizure.
93. The method of embodiment 91, wherein the disease is Alzheimer's disease.
94. The method of embodiment 91, wherein the disease is Huntington's disease.
95. The method of embodiment 91, wherein the disease is Parkinson's disease.
96. The method of embodiment 91, wherein the disease is amyotrophic lateral sclerosis.
97. The method of any one of embodiments 80-83, wherein the disease is cancer.
98. The method of embodiment 97, wherein the disease is a hematological malignancy.
99. The method of embodiment 97, wherein the disease is leukemia.
100. The method of embodiment 97, wherein the disease is acute myeloid leukemia.
101. The method of embodiment 97, wherein the disease is acute lymphoblastic leukemia.
102. The method of embodiment 97, wherein the disease is colon cancer.
103. The method of embodiment 97, wherein the disease is pancreatic cancer.
104. The method of any one of embodiments 80-83, wherein the disease is a metabolic disease.
105. The method of embodiment 104, wherein the disease is diabetes.
106. The method of embodiment 104, wherein the disease is Type II diabetes.
107. The method of embodiment 104, wherein the disease is obesity.
108. The method of any one of embodiments 80-83, wherein the disease is CTNNB1 syndrome.
109. A method of inhibiting the aberrantly high activity and/or production of a glycogen synthase kinase 3 (GSK3) in a subject in need thereof, cell, tissue, or biological sample, the method comprising administering to the subject or contacting the cell, tissue, or biological sample with an effective amount of the compound of any one of embodiments 1-75, or a pharmaceutically acceptable salt, solvate, hydrate, polymorph, co-crystal, tautomer, stereoisomer, isotopically labeled compound, or prodrug thereof, or the pharmaceutical composition of embodiment 77 or 78.
110. The method of any one of embodiments 80-109, wherein the subject is a human.
111. The method of embodiment 110, wherein the subject is a human aged 18 years and older.
112. A method of inhibiting the aberrantly high activity and/or production of a glycogen synthase kinase 3 (GSK3) in a cell, tissue, or biological sample, the method comprising contacting the cell, tissue, or biological sample with an effective amount of the compound of any one of embodiments 1-75, or a pharmaceutically acceptable salt, solvate, hydrate, polymorph, co-crystal, tautomer, stereoisomer, isotopically labeled compound, or prodrug thereof, or the pharmaceutical composition of embodiment 77 or 78, wherein the cell, tissue, or biological sample is in vitro.
113. The method of any one of embodiments 82-112, wherein the GSK3 is glycogen synthase kinase 3 α (GSK3α).
114. The method of embodiment 113, wherein the compound, or pharmaceutically acceptable salt, solvate, hydrate, polymorph, co-crystal, tautomer, stereoisomer, isotopically labeled compound, or prodrug thereof, is at least three times more selective for inhibiting the activity and/or production of GSK3α than glycogen synthase kinase 3 β (GSK3β) in an in vitro assay.
115. The method of any one of embodiments 82-112, wherein the GSK3 is glycogen synthase kinase 3 β (GSK30).
116. The method of embodiment 115, wherein the compound, or pharmaceutically acceptable salt, solvate, hydrate, polymorph, co-crystal, tautomer, stereoisomer, isotopically labeled compound, or prodrug thereof, is at least three times more selective for inhibiting the activity and/or production of GSK3β than glycogen synthase kinase 3 α (GSK3α) in an in vitro assay.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Chemistry General Materials and Methods

Unless otherwise provided:
1. mixtures of stereoisomers were separated by chiral preparatory supercritical fluid chromatography (“SFC”);
2. each of the compounds of the present disclosure is assigned a three- (e.g., 101 or 501) or four- (e.g., 1002) digit compound number, optionally with a one- or two-letter suffix (e.g., R, S, RR, RS, SR, SS, A, B, C, or D);
3. a three- or four-digit compound number, without a one- or two-letter suffix, refers to a mixture of stereoisomers;
4. each of the stereoisomers of the compounds of the present disclosure is assigned into its compound number the letter R or S (where there are two stereoisomers) or RR, RS, SR, or SS (where there are four stereoisomers) according to the stereochemistry; see, e.g., Tables 1 and 2;
5. each of the separated (e.g., separated by SFC) stereoisomers of the compounds of the present disclosure was assigned into its compound number the letter A or B (where there were two stereoisomers) or A, B, C, or D (where there were four stereoisomers) according to the order of elution (e.g., order of elution from the SFC) from the first to last; for example, when the mixture 101 has two stereoisomers, the first eluted stereoisomer of 101 was assigned the compound number 101A, and the second eluted stereoisomer of 101 was assigned the compound number 101B; see, e.g., Table 3 (where the numeric part of each of the compound numbers in Table 3 corresponds to the numeric part of each of the compound numbers in Table 1) and Table 4 (where the numeric part of each of the compound numbers in Table 4 corresponds to the numeric part of each of the compound numbers in Table 2);
6. compound numbers of the compounds of the present disclosure with the same digits refer to compounds having the same structure without regard for stereochemistry;
7. enhanced stereochemistry indicators at chiral centers include:
   a. “abs”: the stereochemistry is absolute and is as shown;
   b. “&”: both the stereochemistry as shown and its opposite stereochemistry;
   c. “or”: single stereoisomer of unknown stereochemistry (i.e., either the stereochemistry as shown or its opposite stereochemistry but not both), even when shown with R, S, [symbol] , and/or [symbol] ; and for example, the enhanced stereochemistry indicators for Compounds 13A and 13B are “or1”, and therefore, Compound 13A is a single stereoisomer of unknown stereochemistry even when its compound name includes “S”, and Compound 13B is a single stereoisomer of unknown stereochemistry even when its compound name includes “R”; and
   d. digit (e.g., “1”, “2”): used to group tags together: those with the same digits (e.g., “or1” and “or1”) move together, and those with different digits (e.g., “or1” and “or2”) move independently.

### Example 1. Synthesis of Select Compounds

The compound numbers recited in each one of Examples 1.1 to 1.57 apply only to each one of Examples 1.1 to 1.57, respectively.

### Example 1.1. (5S)-3-bromo-5,8,8-trimethyl-5-phenyl-9,10-dihydro-7H-benzo[b][1,8]naphthyridin-6-one. (4A)

### (5R)-3-bromo-5,8,8-trimethyl-5-phenyl-9,10-dihydro-7H-benzo[b][1,8]naphthyridin-6-one. (4B)

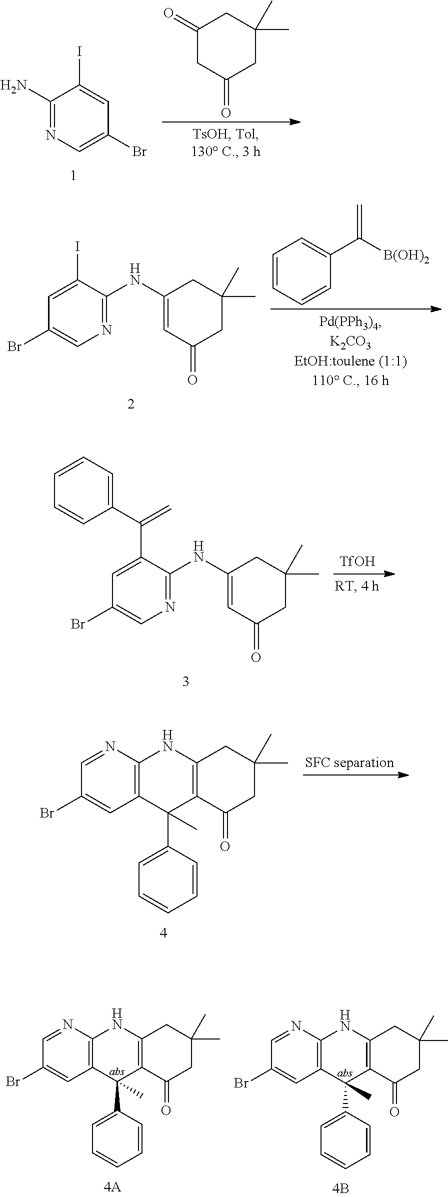

### Step 1: 3-((5-bromo-3-iodopyridin-2-yl)amino)-5,5-dimethylcyclohex-2-en-1-one. (2)

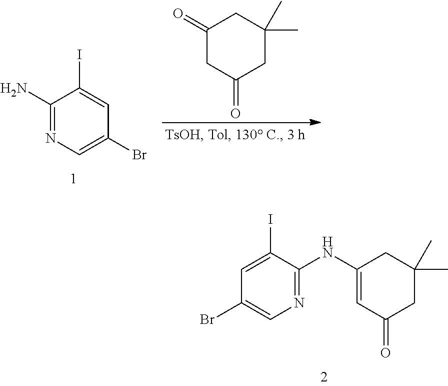

A reaction mixture of compound 1 (20.0 g, 66.9 mmol, 1.00 eq) TsOH (1.15 g, 6.69 mmol, 0.10 eq) and dimedone (1.03 g, 73.6 mmol, 1.10 eq) in Tol (200 mL) was stirred at 130° C. for 3 h. On completion, the reaction mixture was diluted with EtOAc (3×200 mL). The organic layer was washed with brine (3×200 mL), dried over Na₂SO₄, filtered, and concentrated in vacuo. The residue was purified by flash silica gel chromatography (ISCO®; 220 g SepaFlash® Silica Flash Column, Eluent of 0˜10% Ethyl acetate/Petroleum ether gradient @ 100 mL/min) to give 3-((5-bromo-3-iodopyridin-2-yl)amino)-5,5-dimethylcyclohex-2-en-1-one (14.0 g, 50% yield) as a white solid.

LCMS: tR=0.436 min., (ES⁺) m/z (M+H)⁺=421.2.

### Step 2: 3-[[5-bromo-3-(1-phenylvinyl)-2-pyridyl]amino]-5,5-dimethyl-cyclohex-2-en-1-one. (3)

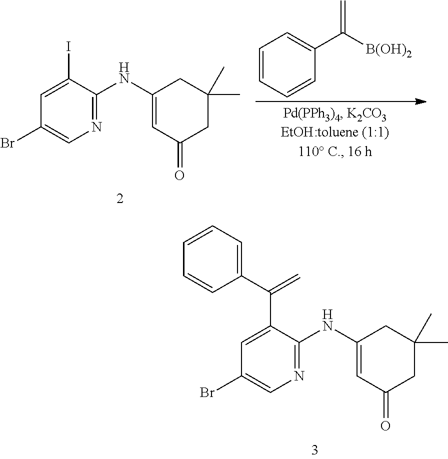

To a round bottom flask 3-((5-bromo-3-iodopyridin-2-yl)amino)-5,5-dimethylcyclohex-2-en-1-one (3000 mg, 7.12 mmol, 1.00 eq), 1-phenylvinylboronic acid (2108 mg, 14.2 mmol, 2.00 eq) and K₂CO₃ (2950 mg, 21.4 mmol, 3.00 eq) were taken in toluene (30 mL) and EtOH (30 mL). The reaction suspension was bubbled with N₂ for 5 min then Pd(PPh₃)₄ (823 mg, 0.712 mmol, 0.100 eq) was added. The reaction mixture was then heated at 110° C. for 16 h and monitored by TLC and LCMS. After completion of the starting material as indicated by TLC, the reaction mixture was concentrated under reduced pressure and mixture was extracted with ethyl acetate, the combined organic layer was washed with brine and dried over anhydrous Na₂SO₄. After filtration and removal of solvent in vacuo, the crude product was purified by column chromatography using 50% EtOAc and Hexane as the eluent to give 3-[[5-bromo-3-(1-phenylvinyl)-2-pyridyl]amino]-5,5-dimethyl-cyclohex-2-en-1-one (1.8 g, 63%).

### Step 3: (5S)-3-bromo-5,8,8-trimethyl-5-phenyl-9,10-dihydro-7H-benzo[b][1,8]naphthyridin-6-one. (4A)

### (5R)-3-bromo-5,8,8-trimethyl-5-phenyl-9,10-dihydro-7H-benzo[b][1,8]naphthyridin-6-one. (4B)

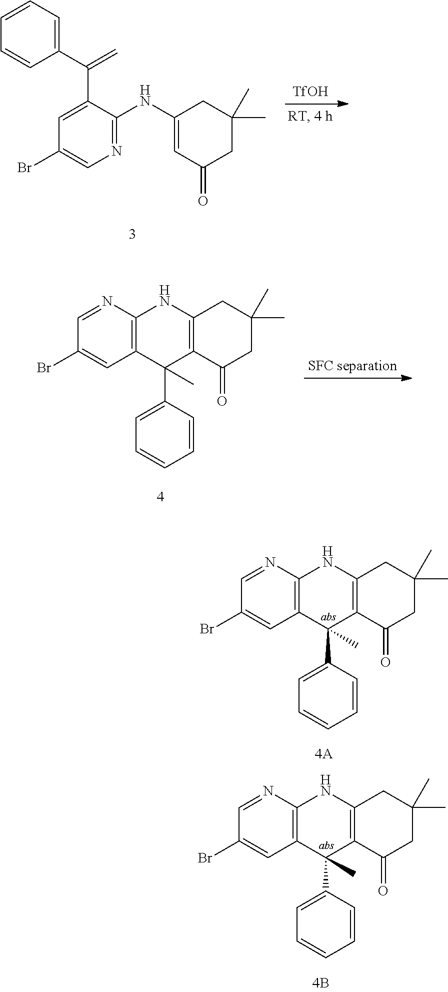

Into a 50 mL RBF containing 3-((5-bromo-3-(1-phenylvinyl)pyridin-2-yl)amino)-5,5-dimethylcyclohex-2-en-1-one (4.30 g, 10.8 mmol, 1.00 eq) was added TfOH (0.96 mL, 10.8 mmol, 1.00 eq) at 25° C. The reaction mixture was stirred at 25° C. for 16 h. The reaction was monitored by TLC analysis. After 16 h, the reaction mixture was quenched with NaHCO₃ solution (200 mL) and extracted with EtOAc (3×200 mL). The combined organic layer was washed with brine solution (120 mL), dried over anhydrous Na₂SO₄, and was concentrated under reduced pressure to obtain a crude residue. The crude residue was purified by column chromatography, eluting with 0-25% EtOAc in Hex to afford 3-bromo-5,8,8-trimethyl-5-phenyl-9,10-dihydro-7H-benzo[b][1,8]naphthyridin-6-one.

The two enantiomers were separated by chiral preparatory SFC (Column (R,R) WHELK-O1 (21 mm×250 mm), 5μ Flow: 50 g/min Mobile Phase: 70% CO₂+30% (0.3% IPamine in Methanol) ABPR: 100 bar, Temp: 35° C. UV: 220 nm diluent: MEOH+DCM):

Peak 1: (1.60 g, 3.95 mmol, 98% purity, 36% yield) (5S)-3-bromo-5,8,8-trimethyl-5-phenyl-9,10-dihydro-7H-benzo[b][1,8]naphthyridin-6-one. (4A)).

LCMS: (ES⁺) m/z (M+H)⁺=399.4, tR=2.00 min.

HPLC: (purity: 98%).

Peak 2: (1.60 g, 3.95 mmol, 98% purity, 36% yield) (5R)-3-bromo-5,8,8-trimethyl-5-phenyl-9,10-dihydro-7H-benzo[b][1,8]naphthyridin-6-one. (4B).

LCMS: tR=1.99 min, (ES⁺) m/z (M+H)⁺=399.3.

HPLC: (purity: 98%).

### Example 1.2. (5S)-3,5,8,8-tetramethyl-5-phenyl-9,10-dihydro-7H-benzo[b][1,8]naphthyridin-6-one. (5)

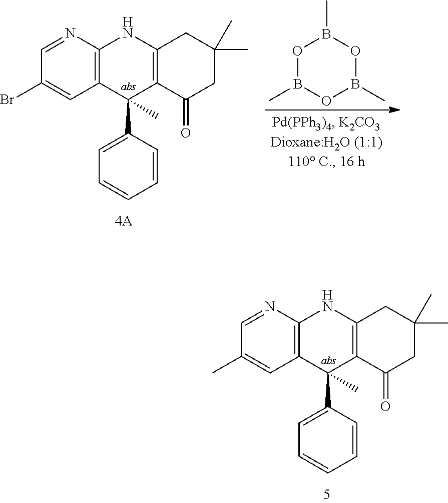

Into a 25 mL seal-tube containing a stirred solution of (5S)-3-bromo-5,8,8-trimethyl-5-phenyl-9,10-dihydro-7H-benzo[b][1,8]naphthyridin-6-one (200 mg, 0.503 mmol, 1.00 eq) and 2,4,6-trimethyl-1,3,5,2,4,6-trioxatriborinane (126 mg, 0.500 mmol, 1.00 eq) in 1,4-dioxane (2.5 mL) and water (2.5 mL) was added K₂CO₃ (208 mg, 1.51 mmol, 3.00 eq) at RT. The reaction mixture was degassed with N₂ for 10 min. After 10 min, Pd(PPh₃)₄ (58 mg, 0.0503 mmol, 0.100 eq) was added and the reaction mixture was stirred at 110° C. for 16 h. The reaction was monitored by LCMS analysis. After 16 h, the reaction mixture was cooled to RT, quenched with water (10 mL), and extracted with EtOAc (3×15 mL). The combined organic layer was washed with brine solution (10 mL), dried over anhydrous Na2SO4, and concentrated under reduced pressure to obtain a crude residue. The crude residue was purified by preparative HPLC (Column: Poroshell 120 EC-C18 (3.0×100 mm), 2.7 um; Mobile Phase: A: ACN B: 0.05% TFA in water. Diluent: DMSO to afford (5S)-3,5,8,8-tetramethyl-5-phenyl-9,10-dihydro-7H-benzo[b][1,8]naphthyridin-6-one, (27 mg, 0.0784 mmol, 96.44% purity, 16% yield) as light-yellow solid.

LCMS: (ES⁺) m/z (M+H)⁺=333.36, tR=1.903 min.

HPLC: (purity: 96.44%).

¹H NMR (400 MHz, DMSO-d6) δ=9.664 (s, 1H), 7.781 (s, 1H), 7.334 (d, J=7.6, 2H), 7.216 (t, J=7.6, 2H), 7.047 (t, J=7.2, 1H), 6.889 (s, 1H), 2.422 (s, 2H), 2.037-1.998 (m, 4H), 1.947-1.901 (m, 4H), 1.008 (d, J=5.2, 6H).

### Example 1.3. (5S)-3-ethyl-5,8,8-trimethyl-5-phenyl-9,10-dihydro-7H-benzo[b][1,8]naphthyridin-6-one. (6)

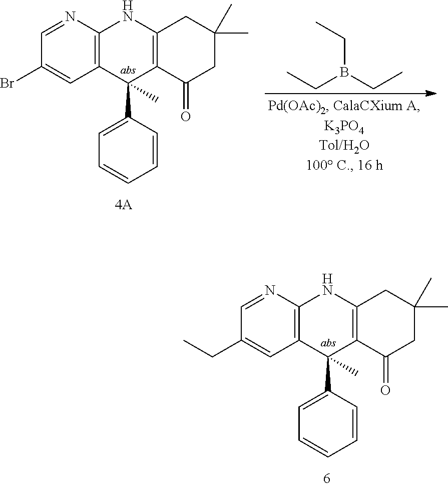

Into a 25 mL seal-tube containing a stirred solution of (5S)-3-bromo-5,8,8-trimethyl-5-phenyl-9,10-dihydro-7H-benzo[b][1,8]naphthyridin-6-one (200 mg, 0.503 mmol, 1.00 eq) and triethyl borane (1 M in THF, 0.60 mL, 0.604 mmol, 1.20 eq) in toluene (6 mL) and water (0.60 mL) was added K₃PO₄ (214 mg, 1.01 mmol, 2.00 eq) at RT. The reaction mixture was degassed with N₂ for 10 min. After 10 min, CataCXium A (36 mg, 0.101 mmol, 0.200 eq) and Pd(OAc)₂ (23 mg, 0.101 mmol, 0.200 eq) were added and the reaction mixture was stirred at 100° C. for 16 h. The reaction was monitored by LCMS analysis. After 16 h, the reaction mixture was cooled to RT and was quenched with water (10 mL) and extracted with EtOAc (3×15 mL). The combined organic layer was washed with brine solution (10 mL), dried over Na₂SO₄, and was concentrated under reduced pressure to obtain a crude residue. The crude residue was purified by preparatory HPLC (Column:—HYDROSPHERE C18 (250×20 mm, 5μ), flow rate: 16.0 ml/min. Mobile phase: A=10 mM NH₄OAC in water) to afford (5S)-3-ethyl-5,8,8-trimethyl-5-phenyl-9,10-dihydro-7H-benzo[b][1,8]naphthyridin-6-one (36 mg, 0.102 mmol, 99.16% purity, 20% yield).

LCMS: tR=347.23 min., (ES⁺) m/z (M+H)⁺=347.23.

HPLC: (purity: 99.16%).

¹H NMR (400 MHz, DMSO-d6) δ=9.697 (s, 1H), 7.814 (s, 1H), 7.339 (d, J=7.6, 2H), 7.216 (t, J=7.6, 2H), 7.046 (t, J=7.2, 1H), 6.906 (s, 1H), 2.468 (s, 2H), 2.364-2.309 (m, 2H), 2.038-1.951 (m, 2H), 1.908 (s, 3H), 1.008-0.963 (m, 9H).

### Example 1.4. (5S)-3-isopropyl-5,8,8-trimethyl-5-phenyl-9,10-dihydro-7H-benzo[b][1,8]naphthyridin-6-one. (7)

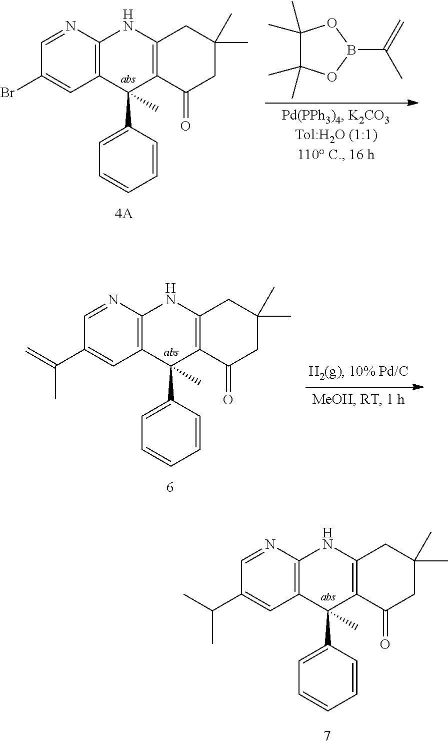

### Step 1: (5S)-3-isopropenyl-5,8,8-trimethyl-5-phenyl-9,10-dihydro-7H-benzo[b][1,8]naphthyridin-6-one. (6)

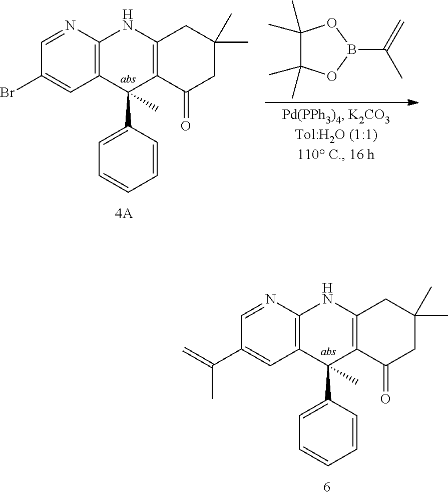

To a 25 mL seal-tube containing a stirred solution of 2-isopropenyl-4,4,5,5-tetramethyl-1,3,2-dioxaborolane (0.16 mL, 0.831 mmol, 1.50 eq) and (5S)-3-bromo-5,8,8-trimethyl-5-phenyl-9,10-dihydro-7H-benzo[b][1,8]naphthyridin-6-one (220 mg, 0.554 mmol, 1.00 eq) in toluene (4 mL) and water (4 mL) (1:1) was added K₂CO₃ (153 mg, 1.11 mmol, 2.00 eq) at RT. The reaction mixture was degassed with nitrogen for 10 min. After 10 min, Pd(PPH₃)₄ (64 mg, 0.0554 mmol, 0.100 eq) was added and the reaction mixture was stirred at 110° C. for 16 h. The reaction was monitored by LCMS analysis. After 16 h, the reaction mixture was cooled to RT and was quenched with water (10 mL) and extracted with ethyl acetate (3×25 mL). The combined organic layer was washed with brine solution (20 mL), dried over anhydrous Na₂SO₄, and was concentrated under reduced pressure to obtain a crude residue. The crude residue was purified by column chromatography, eluting with 0-55% EtOAc in Hexane to afford (5S)-3-isopropenyl-5,8,8-trimethyl-5-phenyl-9,10-dihydro-7H-benzo[b][1,8]naphthyridin-6-one, (120 mg, 0.328 mmol, 98% purity, 59% yield) as pale-yellow solid.

LCMS: (ES⁺) m/z (M+H)⁺=359.2, tR=1.995 min.

### Step 2: (5S)-3-isopropyl-5,8,8-trimethyl-5-phenyl-9,10-dihydro-7H-benzo[b][1,8]naphthyridin-6-one. (7)

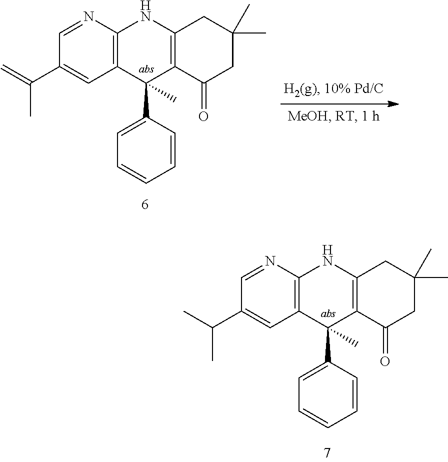 [SMILES: C=C(C)c1cnc2c(c1)[C@](C)(c1ccccc1)C1=C(CC(C)(C)CC1=O)N2 ; CC(C)c1cnc2c(c1)[C@](C)(c1ccccc1)C1=C(CC(C)(C)CC1=O)N2]

Into a 25 mL RB containing a stirred solution of (5S)-3-isopropenyl-5,8,8-trimethyl-5-phenyl-9,10-dihydro-7H-benzo[b][1,8]naphthyridin-6-one (110 mg, 0.307 mmol, 1.00 eq) in methanol (2 mL) was added 10% Pd/C (60 mg) at RT. The reaction mixture was stirred at 25° C. under hydrogen gas pressure for 1 h. The reaction was monitored by LCMS analysis. The reaction mixture was filtered through celite pad and the celite pad was washed with methanol (2×20 mL). The combined organic layer was concentrated under reduced pressure to obtain a crude residue. The crude residue was purified by preparatory HPLC (Column: YMC-Actus Triart C18 (250×20 mm, 5μ) flow rate of 16.0 ml/min. Mobile phase: A=20 mM NH₄HCO₃ in water, B=Acetonitrile) to give (5S)-3-isopropyl-5,8,8-trimethyl-5-phenyl-9,10-dihydro-7H-benzo[b][1,8]naphthyridin-6-one, (53 mg, 0.147 mmol, 99.36% purity, 48% yield) as white solid.

LCMS: tR=2.043 min., (ES⁺) m/z (M+H)⁺=361.25.

HPLC: (purity: 99.36%).

¹H NMR (400 MHz, DMSO-d6) δ=9.713 (s, 1H), 7.846 (s, 1H), 7.343 (d, J=7.6, 2H), 7.217 (t, J=7.6, 2H), 7.047 (t, J=7.6, 1H), 6.916 (s, 1H), 2.688-2.169 (m, 1H), 2.421-2.328 (m, 2H), 2.039-1.958 (m, 2H), 1.908 (s, 3H), 1.037-0.877 (m, 12H).

### Example 1.5. (5S)-3-cyclopropyl-5,8,8-trimethyl-5-phenyl-9,10-dihydro-7H-benzo[b][1,8]naphthyridin-6-one. (8)

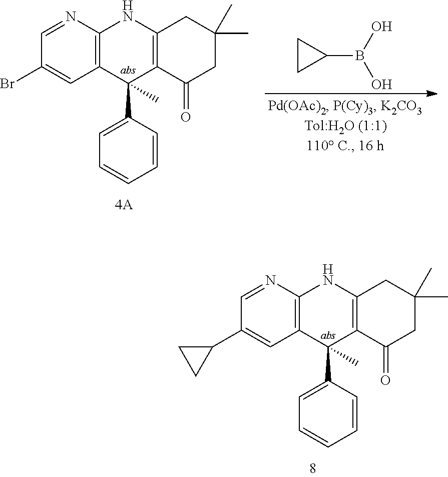 [SMILES: CC1(C)CC(=O)C2=C(C1)Nc1ncc(Br)cc1[C@]2(C)c1ccccc1 ; OB(O)C1CC1 ; CC1(C)CC(=O)C2=C(C1)Nc1ncc(C3CC3)cc1[C@]2(C)c1ccccc1]

To a 25 mL seal-tube containing a stirred solution of (5S)-3-bromo-5,8,8-trimethyl-5-phenyl-9,10-dihydro-7H-benzo[b][1,8]naphthyridin-6-one (200 mg, 0.503 mmol, 1.00 eq) and cyclopropyl boronic acid (130 mg, 1.51 mmol, 3.00 eq) in toluene (4 mL) and water (1 mL) was added K₃PO₄ (320 mg, 1.51 mmol, 3.00 eq) at RT. The reaction mixture was degassed with nitrogen for 10 min. After 10 min, P(Cy)₃ (28 mg, 0.101 mmol, 0.200 eq) and Pd(OAc)₂ (11 mg, 0.0503 mmol, 0.100 eq) were added and the reaction mixture was stirred at 110° C. for 16 h. The reaction was monitored by LCMS analysis. After 16 h, the reaction mixture was cooled to RT and was quenched with water (10 mL) and extracted with ethyl acetate (3×15 mL). The combined organic layer was washed with brine solution (10 mL), dried over anhydrous Na₂SO₄, and was concentrated under reduced pressure to obtain a crude residue. The crude residue was purified by preparatory HPLC (Column: Poroshell 120 EC-C18 (3.0×100 mm), 2.7 um; Mobile Phase: A: ACN B: 0.05% TFA in water; Diluent: DMSO) to afford (5S)-3-cyclopropyl-5,8,8-trimethyl-5-phenyl-9,10-dihydro-7H-benzo[b][1,8]naphthyridin-6-one, (47 mg, 0.128 mmol, 98.41% purity, 25% yield) as light-yellow solid.

LCMS: tR=1.98 min., (ES⁺) m/z (M+H)⁺=359.38.

HPLC: (purity: 98.41%).

¹H NMR (400 MHz, DMSO-d6) δ=9.683 (s, 1H), 7.712 (s, 1H), 7.334 (d, J=8, 2H), 7.218 (t, J=7.6, 2H), 7.050 (t, J=7.2, 1H), 6.745 (s, 1H), 2.463 (s, 2H), 2.036-1.950 (m, 2H), 1.898 (s, 3H), 1.709-1.642 (m, 1H), 1.006 (d, J=4.4, 6H), 0.800-0.779 (m, 2H), 0.497-0.471 (m, 1H), 0.392-0.366 (m, 1H).

### Example 1.6. (5S)-5,8,8-trimethyl-5-phenyl-3-(2,2,2-trifluoroethyl)-9,10-dihydro-7H-benzo[b][1,8]naphthyridin-6-one. (12)

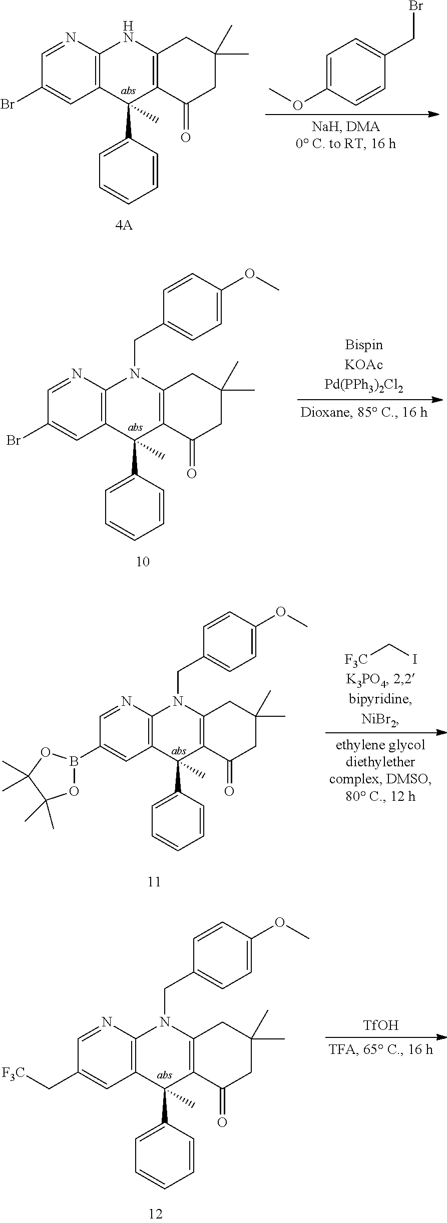

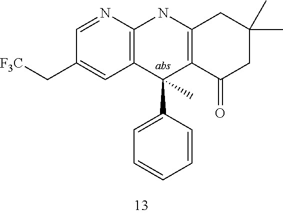 [SMILES: CC1(C)CC(=O)C2=C(C1)Nc1ncc(CC(F)(F)F)cc1[C@]2(C)c1ccccc1]

### Step 1: (5S)-3-bromo-10-[(4-methoxyphenyl)methyl]-5,8,8-trimethyl-5-phenyl-7,9-dihydrobenzo[b][1,8]naphthyridin-6-one. (9)

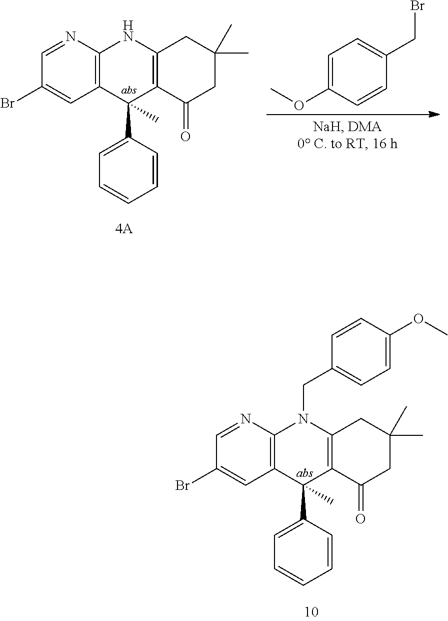

To a stirred solution of (5S)-3-bromo-5,8,8-trimethyl-5-phenyl-9,10-dihydro-7H-benzo[b][1,8]naphthyridin-6-one (250 mg, 0.629 mmol, 1.00 eq) in dimethylacetamide (5 mL) were added 60% sodium hydride (75 mg, 1.89 mmol, 3.00 eq) at 0° C. stirred for 5 min after that added 1-(bromomethyl)-4-methoxy-benzene (0.14 mL, 0.944 mmol, 1.50 eq) resulting mixture was stirred at 25° C. for 16 h. After 16 h reaction was monitor by TLC or LCMS. Mixture was poured into ice cold water; aqueous layer was extracted with EtOAc combined organic layer was give wash of cold water dried over Na₂SO₄ and concentrated under reduced pressure to give crude compound. Crude residue was purified by combi flash chromatography (12 g). The desired product was eluted at 15-20% EtOAc in Hexane to afford (5S)-3-bromo-10-[(4-methoxyphenyl)methyl]-5,8,8-trimethyl-5-phenyl-7,9-dihydrobenzo[b][1,8]naphthyridin-6-one, (200 mg, 0.290 mmol, 75.045% purity, 46% yield).

LCMS: (ES⁺) m/z (M+H)⁺=517.0, tR=4.29 min.

### Step 2: (5S)-10-[(4-methoxyphenyl)methyl]-5,8,8-trimethyl-5-phenyl-3-(4,4,5,5-tetramethyl-1,3,2-dioxaborolan-2-yl)-7,9-dihydrobenzo[b][1,8]naphthyridin-6-one. (10)

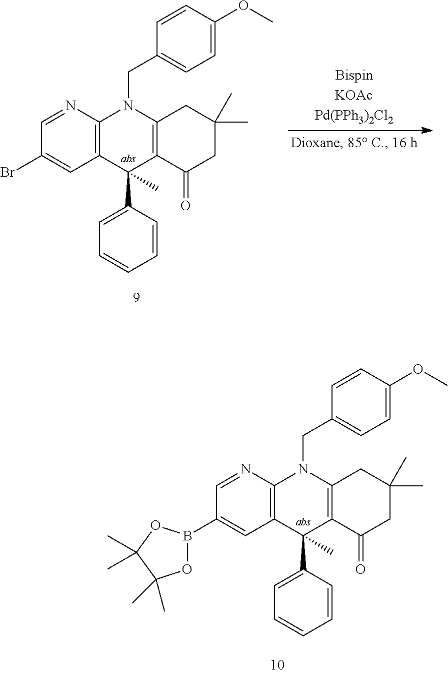

To a stirred solution of (5S)-3-bromo-10-[(4-methoxyphenyl)methyl]-5,8,8-trimethyl-5-phenyl-7,9-dihydrobenzo[b][1,8]naphthyridin-6-one (300 mg, 0.580 mmol, 1.00 eq) in Dioxane (15 mL) and H₂O (3 mL) were added Bispin (221 mg, 0.870 mmol, 1.50 eq) K₂CO₃ (171 mg, 1.74 mmol, 3.00 eq) degassed with Ar for 15 min added Pd(PPh₃)₂Cl₂ (41 mg, 0.0580 mmol, 0.100 eq) degassed with Ar again 5 min, resulting mixture was heated at 85° C. for 16 h. After 16 h reaction was monitor by TLC or LCMS. Mixture was filter through cartridge diluted with water and extracted with EA combined organic layer was dried over Na₂SO₄ and concentrated under reduced pressure to get crude compound. LCMS showed desired compound mass with boronic acid mass. Crude material was taken to the next step without further purification.

LCMS: tR=10.83 min., (ES⁺) m/z (M+H)⁺=565.4.

### Step 3: (5S)-10-[(4-methoxyphenyl)methyl]-5,8,8-trimethyl-5-phenyl-3-(2,2,2-trifluoroethyl)-7,9-dihydrobenzo[b][1,8]naphthyridin-6-one. (11)

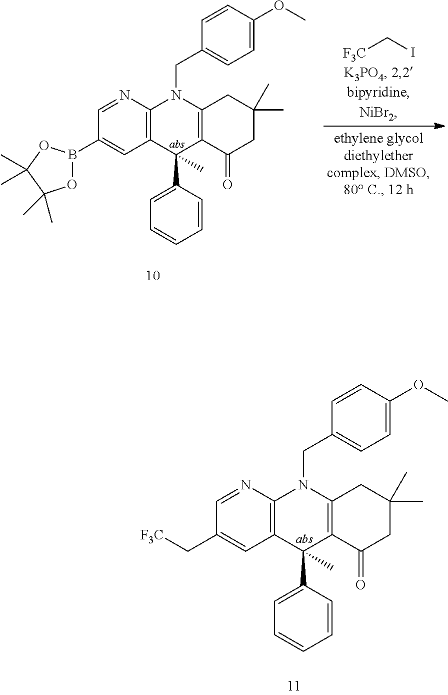

To a 25 mL RBF were added (5R)-10-[(4-methoxyphenyl)methyl]-5,8,8-trimethyl-5-phenyl-3-(4,4,5,5-tetramethyl-1,3,2-dioxaborolan-2-yl)-7,9-dihydrobenzo[b][1,8]naphthyridin-6-one (100 mg, 0.177 mmol, 1.00 eq) nickel(II) bromide, ethylene glycol dimethyl ether (5.5 mg, 0.0177 mmol, 0.100 eq), 4,4 tertbutyl 2,2 bipyridyl (9.5 mg, 0.0354 mmol, 0.200 eq), K₃PO₄ (0.13 mL, 0.443 mmol, 2.50 eq). The tube was then evacuated and refilled with argon for three times. Anhydrous DMSO (5 mL) was added, followed by addition of anhydrous solution of CH₃CH₂I (45 mg, 0.213 mmol, 1.20 eq) in DMSO (2 mL) under Ar. The reaction mixture was stirred at 80° C. for 12 h. Progress of the reaction was monitored by LCMS. LCMS showed that the desired compound was formed. The reaction mixture was taken in water and extracted with EtOAc three times. Combined organic layers were dried over Na₂SO₄. The crude compound was purified using combiflash using Ethyl acetate/hex as an eluent. Compound was eluted at 30% EtOAc/Hex. Solvent was concentrated under rotavapor to afford 50 mg (20%) pure compound.

LCMS: (ES⁺) m/z (M+H)⁺=521.0, tR=9.95 min.

### Step 4: (5S)-5,8,8-trimethyl-5-phenyl-3-(2,2,2-trifluoroethyl)-9,10-dihydro-7H-benzo[b][1,8]naphthyridin-6-one. (12)

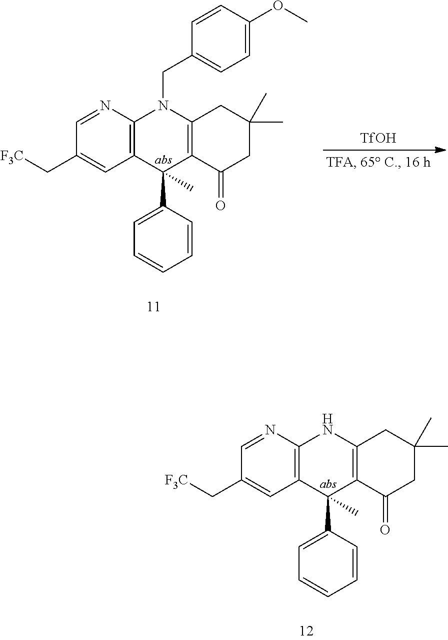

To a stirred solution of (5S)-10-[(4-methoxyphenyl)methyl]-5,8,8-trimethyl-5-phenyl-3-(2,2,2-trifluoroethyl)-7,9-dihydrobenzo[b][1,8]naphthyridin-6-one (6.0 mg, 0.0115 mmol, 1.00 eq) was taken in TFA (2 mL) then the reaction mixture was cooled at 0° C., then triflic acid (0.01 mL, 0.0692 mmol, 6.00 eq) was added the reaction mixture dropwise. The reaction mixture was placed at 60° C. heating condition for 16 hours. Progress of the reaction was monitored by LCMS. As per LCMS desired compound mass was observed ˜30%. The reaction mixture was evaporated under rotavapor to get crude material, the crude material was washed with n-pentane and submit in prep HPLC for isolate the desired peak. After prep HPLC (Column: Luna omega polar C18 (100×4.6 mm); 5u, Diluent: DMSO; Mobile phase: A: 0.05% TFA in Water: B: ACN). (5S)-5,8,8-trimethyl-5-phenyl-3-(2,2,2-trifluoroethyl)-9,10-dihydro-7H-benzo[b][1,8]naphthyridin-6-one, (0.77 mg, 0.00192 mmol, 99.66% purity, 17% yield) was afforded as a white solid compound.

LCMS: (ES⁺) m/z (M+H)⁺=400.18, tR=2.80 min.

HPLC: (purity: 99.66 at 214 nm).

¹H NMR (400 MHz, DMSO-d6) δ=1.010 (s, 6H), 1.900 (s, 3H), 2.009 (s, 2H), 2.500 (s, 2H), 3.420 (s, 2H), 7.05 (d, 2H), 7.223 (m, 2H), 7.312 (m, 2H), 7.918 (s, 1H), 9.857 (s, 1H).

¹⁹F-NMR=−65.025 ppm.

### Example 1.7. (5S)-3-cyclobutyl-5,8,8-trimethyl-5-phenyl-9,10-dihydro-7H-benzo[b][1,8]naphthyridin-6-one. (14)

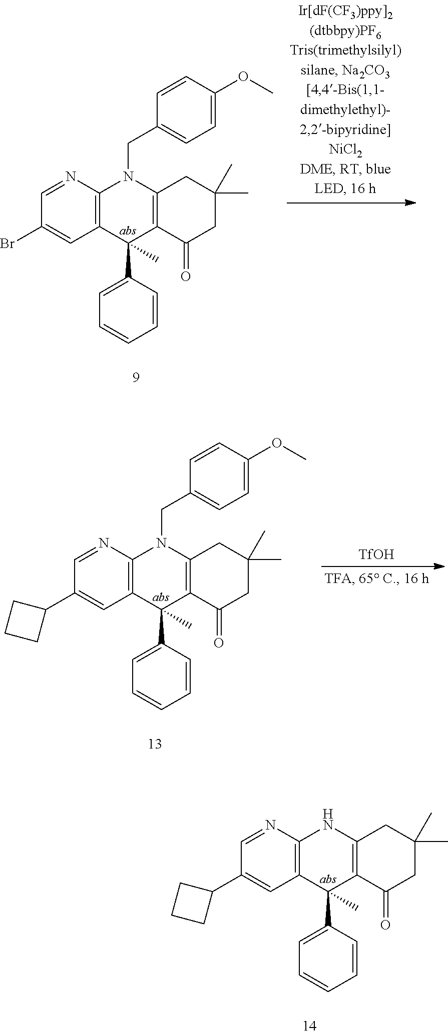

### Step 1: (5S)-4-cyclobutyl-10-[(4-methoxyphenyl)methyl]-5,8,8-trimethyl-5-phenyl-7,9-dihydrobenzo[b][1,8]naphthyridin-6-one. (13)

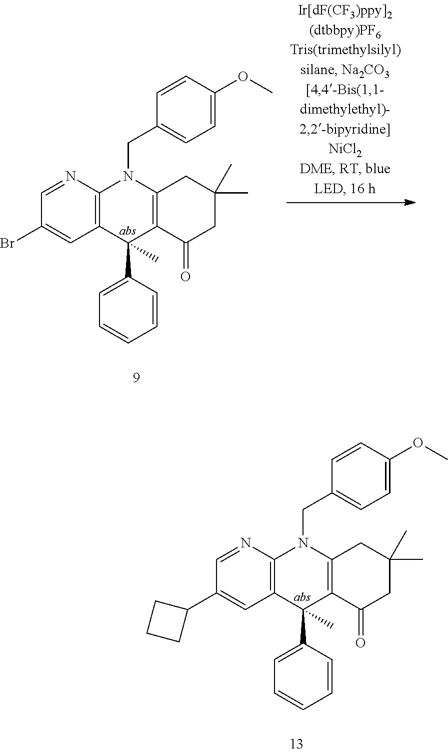 [SMILES: COc1ccc(CN2C3=C(C(=O)CC(C)(C)C3)[C@@](C)(c3ccccc3)c3cc(Br)cnc32)cc1 ; COc1ccc(CN2C3=C(C(=O)CC(C)(C)C3)[C@@](C)(c3ccccc3)c3cc(C4CCC4)cnc32)cc1]

To a stirred solution of (5S)-3-bromo-10-[(4-methoxyphenyl)methyl]-5,8,8-trimethyl-5-phenyl-7,9-dihydrobenzo[b][1,8]naphthyridin-6-one (50 mg, 0.0966 mmol, 1.00 eq) in dimethoxyethane (3 mL) were added bromocyclobutane (20 mg, 0.145 mmol, 1.50 eq), Sodium carbonate (20 mg, 0.193 mmol, 2.00 eq), tris(trimethylsilyl)silane (0.03 mL, 0.0966 mmol, 1.00 eq) Ir[dF(CF3)ppy]2(dtbpy)PF6 (1.1 mg, 0.000966 mmol, 0.0100 eq), degassed with Ar for 5 min, after that added [4,4′-Bis(1,1-dimethylethyl)-2,2′-bipyridine]nickel (II) dichloride (0.19 mg, 0.000483 mmol, 0.00500 eq) again degassed for 5 min, resulting mixture was allow to stirred under photoreactor (Blue light) at 25° C. for 16 h. After 16 h reaction was monitor by TLC or LCMS.

LCMS: (ES⁺) m/z (M+H)⁺=493.7.

### Step 2: (5S)-3-cyclobutyl-5,8,8-trimethyl-5-phenyl-9,10-dihydro-7H-benzo[b][1,8]naphthyridin-6-one. (14)

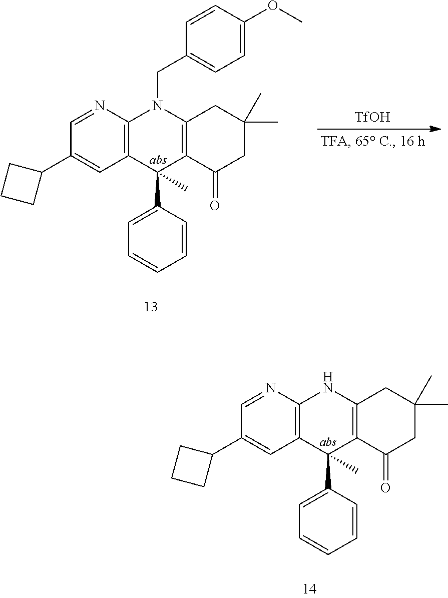

To a stirred solution of (5S)-3-cyclobutyl-10-[(4-methoxyphenyl)methyl]-5,8,8-trimethyl-5-phenyl-7,9-dihydrobenzo[b][1,8]naphthyridin-6-one (90 mg, 0.183 mmol, 1.00 eq) in trifluoroacetic acid (3 mL) were added Triflic acid (0.1 mL, 1.10 mmol, 6.00 eq) resulting mixture was heated at 65° C. for 16 h. After 16 h reaction was monitored by TLC or LCMS. TLC: 50% EA in hexane shows formation of desired product. Mixture was poured into ice cold water diluted with DCM, aqueous layer was extracted with 10% MeOH in DCM combined organic layer was dried over Na₂SO₄ and concentrated under reduce pressure to get crude compound. Crude residue was purified by prep HPLC (Column: YMC-Actus Triart C18 (250×20 mm, 5μ) operating at ambient temperature, flow rate of 16.0 ml/min. Mobile phase: A=20 mM NH₄HCO₃ in water, B=Acetonitrile) to afford (5S)-3-cyclobutyl-5,8,8-trimethyl-5-phenyl-9,10-dihydro-7H-benzo[b][1,8]naphthyridin-6-one (13 mg, 0.0323 mmol, 96.07% purity, 18% yield).

LCMS: tR=3.15 min., (ES⁺) m/z (M+H)⁺=373.36.

HPLC: (purity: 96.07%).

¹H NMR (400 MHz, DMSO-d₆) δ=0.998 (s, 6H), 1.703 (s, 1H), 1.862 (s, 7H), 2.042 (s, l H) 2.132 (s, 2H), 2.500 (d, 2H) 3.313 (m, 1H), 6.943 (d, 1H), 7.069 (t, 1H), 7.240 (t, 2H), 7.347 (d, 2H), 7.847 (s, 1H), 9.723 (s, 1H).

### Example 1.8. (S)-5,8,8-trimethyl-6-oxo-5-phenyl-5,6,7,8,9,10-hexahydrobenzo[b][1,8]naphthyridine-3-carbonitrile (13A) and (R)-5,8,8-trimethyl-6-oxo-5-phenyl-5,6,7,8,9,10-hexahydrobenzo[b][1,8]naphthyridine-3-carbonitrile. (13B)

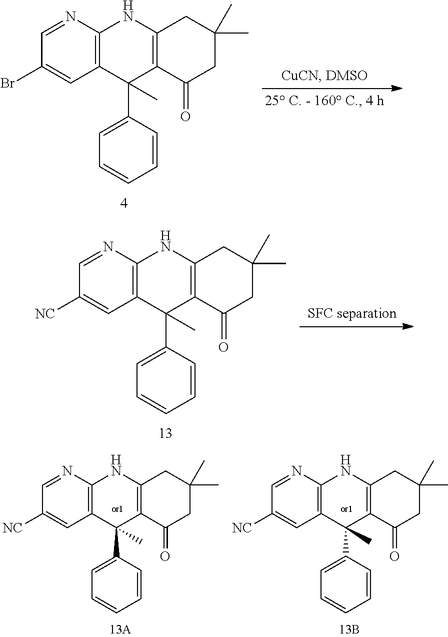 [SMILES: CC1(C)CC(=O)C2=C(C1)Nc1ncc(Br)cc1C2(C)c1ccccc1 ; CC1(C)CC(=O)C2=C(C1)Nc1ncc(C#N)cc1C2(C)c1ccccc1 ; CC1(C)CC(=O)C2=C(C1)Nc1ncc(C#N)cc1[C@]2(C)c1ccccc1 ; CC1(C)CC(=O)C2=C(C1)Nc1ncc(C#N)cc1[C@@]2(C)c1ccccc1]

To a solution of compound 4 (205 mg, 0.51 nmol, 1.00 eq) in DMSO (5 mL) was added CuCN (227 mg, 2.58 mmol, 5.00 eq) portion wise to the reaction mixture at 25° C. The reaction mixture was stirred at 160° C. for 4 h. On completion, the reaction mixture was washed with NH₃·H₂O. The reaction mixture was diluted with EtOAc (3×15 mL). The organic layer was washed with brine (2×15 mL), dried over Na₂SO₄, filtered, and concentrated in vacuo. The residue was purified by prep-HPLC to give compound 5 (100 mg, 56% yield) as a yellow solid.

The yellow product 100 mg was separated by SFC (Column: Chiralcel OJ-3 50×4.6 mm I.D., 3 um Mobile phase: Phase A for CO₂, and Phase B for MeOH (0.05% DEA); Gradient elution: B in A from 5% to 40%; Flow rate: 3 mL/min; Detector: PDA; Column Temp: 35 C; Back Pressure: 100 Bar) to:

Peak 1: (S)-5,8,8-trimethyl-6-oxo-5-phenyl-5,6,7,8,9,10-hexahydrobenzo[b][1,8] naphthyridine-3-carbonitrile (13A) (28.7 mg, 28% yield).

LCMS: tR=1.192 min., (ES⁺) m/z (M+H)⁺=344.0.

¹H NMR (400 MHz, DMSO-d₆) δ=10.32 (s, 1H), 8.40 (d, J=2.4 Hz, 1H), 7.50 (d, J=2.4 Hz, 1H), 7.33 (d, J=0.8 Hz, 1H), 7.35 (d, J=1.2 Hz, 1H), 7.29-7.21 (m, 2H), 7.14-7.03 (m, 1H), 2.57-2.51 (m, 2H), 2.08-1.96 (m, 2H), 1.93 (s, 3H), 1.01 (d, J=3.6 Hz, 6H).

Peak 2: (R)-5,8,8-trimethyl-6-oxo-5-phenyl-5,6,7,8,9,10-hexahydrobenzo[b][1,8]naphthyridine-3-carbonitrile (13B). (27 mg, 27% yield).

LCMS: tR=1.525 min., (ES⁺) m/z (M+H)⁺=344.0.

¹H NMR (400 MHz, DMSO-d₆) δ=10.31 (br s, 1H), 8.40 (d, J=2.4 Hz, 1H), 7.50 (d, J=2.4 Hz, 1H), 7.37-7.31 (m, 2H), 7.30-7.22 (m, 2H), 7.15-7.04 (m, 1H), 2.59-2.51 (m, 2H), 2.08-1.96 (m, 2H), 1.93 (s, 3H), 1.01 (d, J=3.2 Hz, 6H).

### Example 1.9. (5S)-3-tert-butyl-5,8,8-trimethyl-5-phenyl-9,10-dihydro-7H-benzo[b][1,8]naphthyridin-6-one (5A) and (5R)-3-tert-butyl-5,8,8-trimethyl-5-phenyl-9,10-dihydro-7H-benzo[b][1,8]naphthyridin-6-one. (5B)

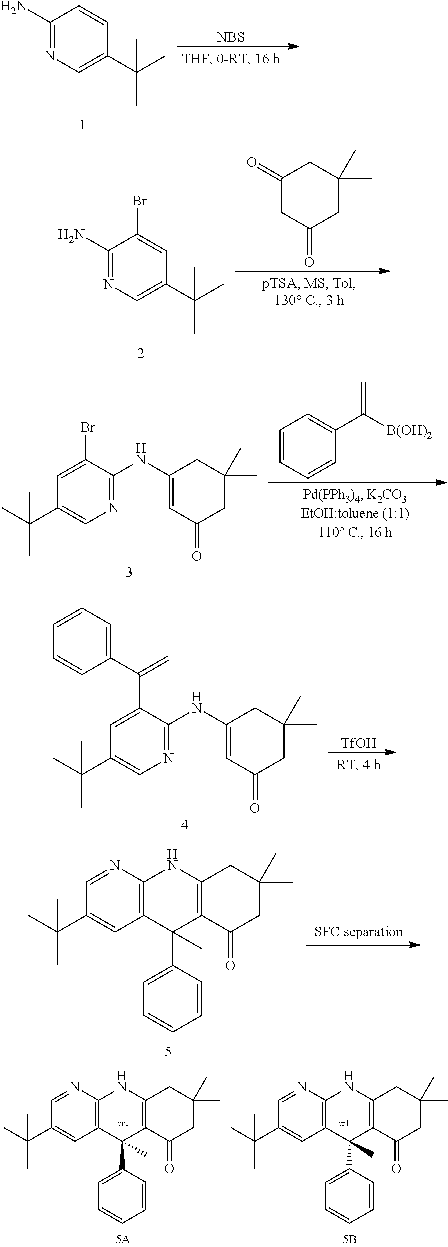

### Step 1: 3-bromo-5-tert-butyl-pyridin-2-amine. (2)

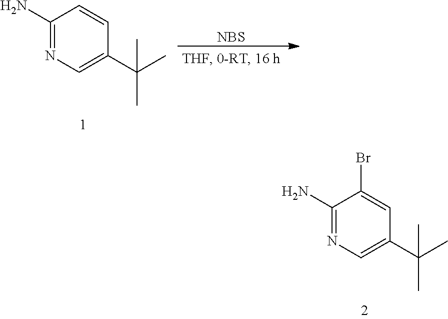

Into a 50 mL RB containing a stirred solution of 5-tert-butylpyridin-2-amine (500 mg, 3.33 mmol, 1.00 eq) in tetrahydrofuran (10 mL) was added NBS (592 mg, 3.33 mmol, 1.0) eq) at 0 C. The reaction mixture was gradually brought to 25° C. and was stirred at 25° C. under N₂ for 16 h. The reaction was monitored by TLC analysis. After 16 h, the reaction mixture was quenched with NaHCO₃ solution (10 mL) and extracted with ethyl acetate (3×20 mL). The combined organic layer was washed with brine solution (10 mL), dried over anhydrous Na₂SO₄ and was concentrated under reduced pressure to obtain a crude residue. The crude residue was purified by column chromatography, eluting with 0-25% ethyl acetate in hexane to afford 3-bromo-5-tert-butyl-pyridin-2-amine, (653 mg, 2.82 mmol, 99% purity, 85% yield) as pale-yellow solid.

LCMS: (ES⁺) m/z (M+H)⁺=231.2, tR=1.674 min.

### Step 2: 3-[(3-bromo-5-tert-butyl-2-pyridyl)amino]-5,5-dimethyl-cyclohex-2-en-1-one. (3)

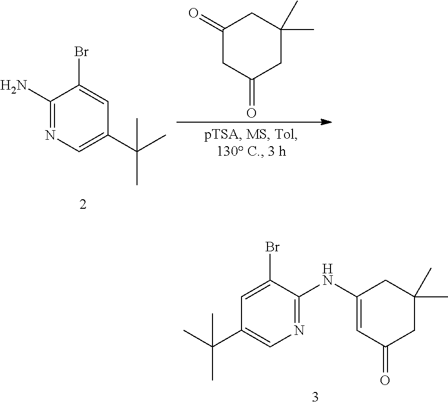

Into a 100 mL RB containing a stirred solution of 3-bromo-5-tert-butyl-pyridin-2-amine (650 mg, 2.84 mmol, 1.00 eq) and 5,5-dimethylcyclohexane-1,3-dione (477 mg, 3.40 mmol, 1.20 eq) in toluene (15 mL) was added Molecular Sieves 4 A (500 mg) and PTSA hydrate (486 mg, 2.55 mmol, 0.900 eq) at RT. The reaction mixture was refluxed at 130° C. for 3 h. The reaction was monitored by TLC analysis. After 16 h, the reaction mixture was cooled to RT, quenched with NaHCO₃ solution (15 mL), and extracted with ethyl acetate (3×20 mL). The combined organic layer was washed with brine solution (10 mL), dried over anhydrous Na₂SO₄, and was concentrated under reduced pressure to obtain a crude residue. The crude residue was purified by column chromatography, eluting with 0-50% ethyl acetate in hexane to afford 3-[(3-bromo-5-tert-butyl-2-pyridyl)amino]-5,5-dimethyl-cyclohex-2-en-1-one, (520 mg, 1.40 mmol, 94.6% purity, 49% yield) as yellow solid.

LCMS: tR=1.976 min., (ES⁺) m/z (M+H)⁺=353.05.

### Step 3: 3-[[5-tert-butyl-3-(1-phenylvinyl)-2-pyridyl]amino]-5,5-dimethyl-cyclohex-2-en-1-one. (4)

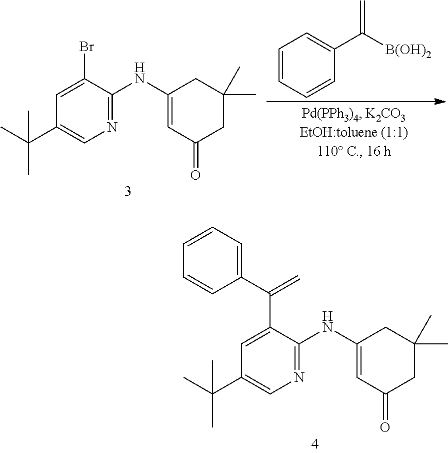

Into a 50 mL RB containing a stirred solution of 3-[(3-bromo-5-tert-butyl-2-pyridyl)amino]-5,5-dimethyl-cyclohex-2-en-1-one (500 mg, 1.42 mmol, 1.00 eq) and 1-phenylvinylboronic acid (211 mg, 1.42 mmol, 1.00 eq) in toluene (8 mL) and ethanol (8 mL) (1:1) was added K₂CO₃ (393 mg, 2.85 mmol, 2.00 eq) at RT. The reaction mixture was degassed with nitrogen for 10 min. After 10 min, Pd(PPH₃)₄ (164 mg, 0.142 mmol, 0.100 eq) was added and the reaction mixture was stirred at 110° C. for 16 h. The reaction was monitored by TLC analysis. After 16 h, the reaction mixture was cooled to RT and was concentrated under reduced pressure to remove ethanol. Then the reaction mixture was quenched with water (20 mL) and extracted with ethyl acetate (3×30 mL). The combined organic layer was washed with brine solution (20 mL), dried over anhydrous Na₂SO₄, and was concentrated under reduced pressure to obtain a crude residue.

The crude residue was purified by column chromatography, eluting with 0-55% ethyl acetate in hexane to afford 3-[[5-tert-butyl-3-(1-phenylvinyl)-2-pyridyl]amino]-5,5-dimethyl-cyclohex-2-en-1-one, (430 mg, 0.919 mmol, 80% purity, 65% yield) as yellow solid.

LCMS: tR=375.36 min., (ES⁺) m/z (M+H)⁺=375.36.

### Step 3: (5S)-3-tert-butyl-5,8,8-trimethyl-5-phenyl-9,10-dihydro-7H-benzo[b][1,8]naphthyridin-6-one (5A) and (5R)-3-tert-butyl-5,8,8-trimethyl-5-phenyl-9,10-dihydro-7H-benzo[b][1,8]naphthyridin-6-one. (5B)

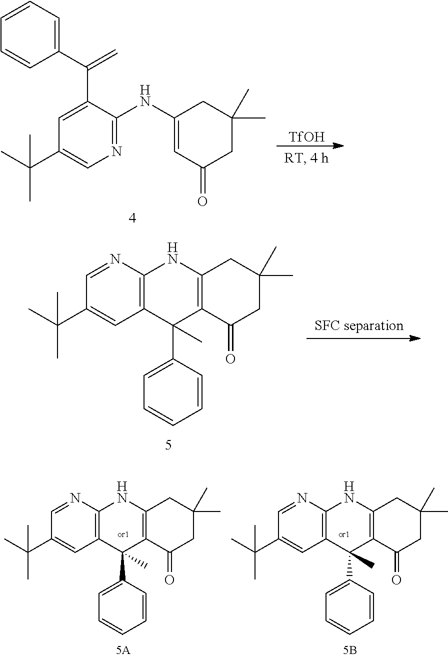

Into a 50 mL RB containing 3-[[5-tert-butyl-3-(1-phenylvinyl)-2-pyridyl]amino]-5,5-dimethyl-cyclohex-2-en-1-one (400 mg, 1.07 mmol, 1.00 eq) was added TfOH (0.09 mL, 1.07 mmol, 1.00 eq) at 0° C. The reaction mixture was stirred at 25° C. for 4 h. The reaction was monitored by TLC analysis. After 4 h, the reaction mixture was quenched with NaHCO₃ solution (20 mL) and extracted with ethyl acetate (3×30 mL). The combined organic layer was washed with brine solution (20 mL), dried over anhydrous Na₂SO₄, and was concentrated under reduced pressure to obtain a crude residue. The crude residue was purified by column chromatography, eluting with 0-25% ethyl acetate in hexane to afford 5 as white solid.

The two enantiomers were separated by chiral preparatory SFC (Column: (R,R) WHELK-(21 mm×250 mm), 5μ Flow: 25 g/min co-solvent: 50% CO₂+50% (0.3% Isopropyl Amine in Methanol) ABPR: 100 bar Temp: 35° C. UV: 215 nm diluent: Methanol):

Peak 1: (5S)-3-tert-butyl-5,8,8-trimethyl-5-phenyl-9,10-dihydro-7H-benzo[b][1,8]naphthyridin-6-one (5A) (25 mg, 0.0660 mmol, 99.28% purity, 6% yield).

LCMS: tR=2.431 min., (ES⁺) m/z (M+H)⁺=375.2.

HPLC: (purity: 99.28%).

¹H NMR (400 MHz, DMSO-d6) δ=1.012 (s, 6H) 1.077 (s, 9H) 1.932 (s, 3H) 2.043 (d, 2H) 2.500 (d, 2H) 7.069 (t, 2H) 7.239 (t, 2H) 7.351 (d, 2H) 7.969 (d, 1H) 9.726 (s, 1H).

Peak 2: (5R)-3-tert-butyl-5,8,8-trimethyl-5-phenyl-9,10-dihydro-7H-benzo[b][1,8]naphthyridin-6-one. (5B) (23 mg, 0.0622 mmol, 99.83% purity, 6% yield).

LCMS: tR=2.109 min., (ES⁺) m/z (M+H)⁺=375.28.

HPLC: (purity: 99.83%).

¹H NMR (400 MHz, DMSO-d6) δ=1.012 (s, 6H) 1.077 (s, 9H) 1.932 (s, 3H) 2.043 (d, 2H) 2.500 (d, 2H) 7.050 (t, 2H) 7.239 (t, 2H) 7.351 (d, 2H) 7.970 (d, 1H) 9.727 (s, 1H).

### Example 1.10. (5S)-3-(2,2-dimethylpropyl)-5,8,8-trimethyl-5-phenyl-9,10-dihydro-7H-benzo[b][1,8]naphthyridin-6-one. (5A) & (5R)-3-(2,2-dimethylpropyl)-5,8,8-trimethyl-5-phenyl-9,10-dihydro-7H-benzo[b][1,8]naphthyridin-6-one. (5B)

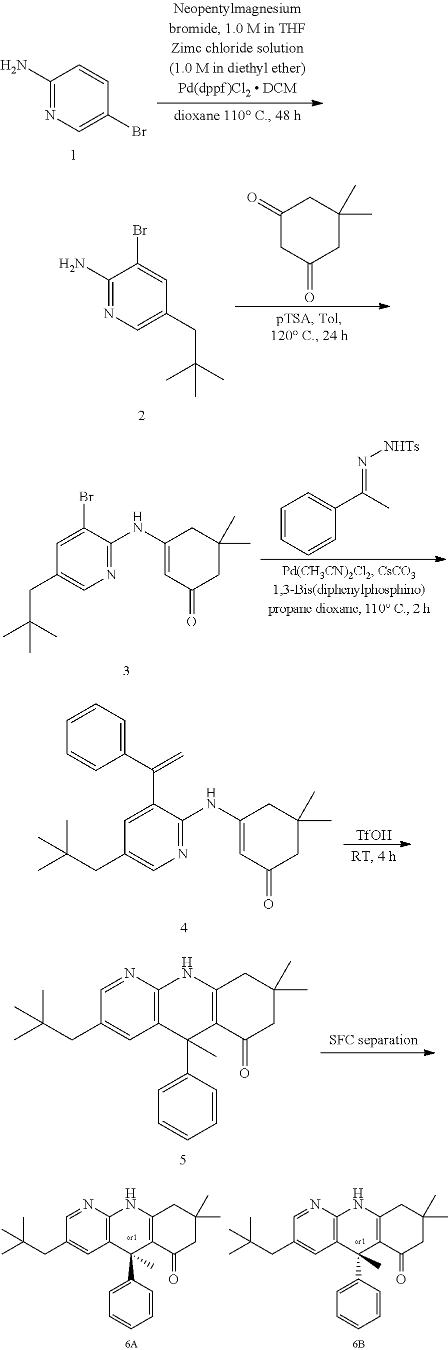

### Step 1: 5-(2,2-dimethylpropyl)pyridin-2-amine. (2)

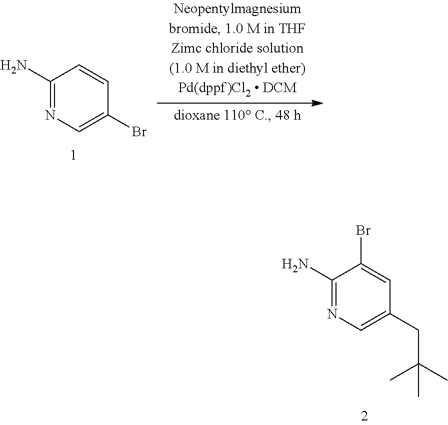

Neopentylmagnesium bromide, 1.0 M in THF (1 M in, 46.24 mL, 46.2 mmol, 4.00 eq) was added to a solution of Zinc chloride solution (1.0 M in diethyl ether) (1 M in, 23.12 mL, 23.1 mmol, 2.00 eq) under argon at 0° C. The solution was diluted with 1,4-dioxane (20 mL) and transferred into a suspension of 5-bromopyridin-2-amine (2.00 g, 11.6 mmol, 1.00 eq) and Pd(dppf)Cl₂·DCM (0.19 g, 0.231 mmol, 0.0200 eq) in 1,4-dioxane (10 mL). The mixture was heated at 110° C. for 48 h. After cooling to room temperature, the mixture was poured into water (50 mL) and aqueous NaHCO₃ (1 M; 40 mL) was added. The mixture was extracted with EtOAc (3×50 mL). The combined organic phases were washed with brine, dried over Na₂SO₄, and concentrated to give crude product which was purified by silica gel chromatography using 20% EtOAc in hexane to provide 5-(2,2-dimethylpropyl)pyridin-2-amine (0.60 g, 3.60 mmol, 98.45% purity, 31% yield).

LCMS: (ES⁺) m/z (M+H)⁺=165.1.

### Step 2: 5-(2,2-dimethylpropyl)pyridin-2-amine. (3)

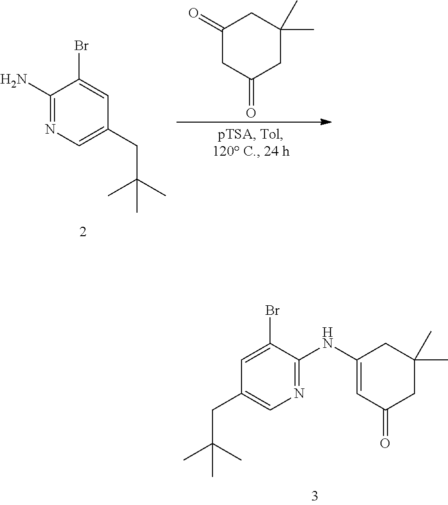

Bromine (0.14 mL, 2.64 mmol, 1.10 eq) was added to a stirred solution of 5-(2,2-dimethylpropyl)pyridin-2-amine (0.40 g, 2.40 mmol, 1.00 eq) and Sodium acetate (0.35 g, 4.32 mmol, 1.80 eq) in acetic acid (4 mL) at 25° C. The warm solution was stirred for 4 h. The dark brown solution was diluted with water (100 mL) and decolorized with sodium sulfite and product was extracted with EtOAc (2×25 mL), organic layers combined, washed with brine, dried over sodium sulfate and evaporated to provide crude product, which was purified by silica gel chromatography using 10% EtOAc in hexane to provide 5-(2,2-dimethylpropyl)pyridin-2-amine (400 mg, 1.53 mmol, 92.95% purity, 64% yield).

LCMS: (ES⁺) m/z (M+H)⁺=245.1.

### Step 3: 3-[[3-bromo-5-(2,2-dimethylpropyl)-2-pyridyl]amino]-5,5-dimethyl-cyclohex-2-en-1-one. (4)

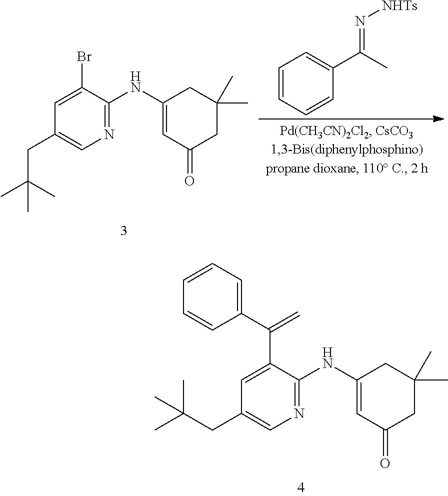

A solution of 3-bromo-5-(2,2-dimethylpropyl)pyridin-2-amine (0.40 g, 1.65 mmol, 1.00 eq)₃-bromo-5-(2,2-dimethylpropyl)pyridin-2-amine (0.40 g, 1.65 mmol, 1.00 eq), 5,5-dimethylcyclohexane-1,3-dione (0.23 g, 1.65 mmol, 1.00 eq) and pTSA (0.085 g, 0.494 mmol, 0.300 eq) in toluene (2.8463 mL) was stirred at 120° C. for 24 h. Progress of reaction was monitored by TLC using Ethyl acetate:Hexanes (2:8). After completion of the rection, ethyl acetate was added and organic layer was washed with saturated NaHCO₃ solution (100 mL×2), water (100 mL) and brine (100 mL), dried over anhydrous Na₂SO₄, and concentrated under vacuum to provide crude product, which was purified by silica gel chromatography using 100% ethyl acetate to afford 3-[[3-bromo-5-(2,2-dimethylpropyl)-2-pyridyl]amino]-5,5-dimethyl-cyclohex-2-en-1-one (370 mg, 1.00 mmol, 99% purity, 61% yield) as a light-yellow solid.

LCMS: (ES⁺) m/z (M+H)⁺=367.2.

### Step 4: (5S)-3-(2,2-dimethylpropyl)-5,8,8-trimethyl-5-phenyl-9,10-dihydro-7H-benzo[b][1,8]naphthyridin-6-one. (5A)

### (5R)-3-(2,2-dimethylpropyl)-5,8,8-trimethyl-5-phenyl-9,10-dihydro-7H-benzo[b][1,8]naphthyridin-6-one. (5B)

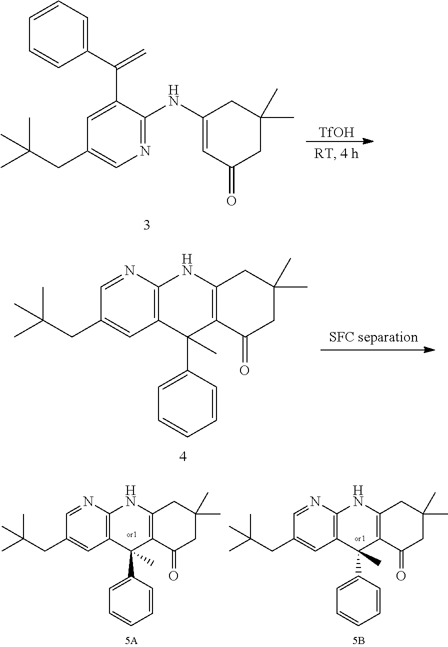

3-[[5-(2,2-dimethylpropyl)-3-(1-phenylvinyl)-2-pyridyl]amino]-5,5-dimethyl-cyclohex-2-en-1-one (0.25 g, 0.643 mmol, 1.0) eq) was dissolved in Trifluoromethanesulfonic acid (0.23 mL, 2.58 mmol, 4.01 eq). The resulting mixture was stirred at 25° C. for 2 h. Reaction progress was monitored by TLC EtOAc:Hexanes (1:1). After completion of reaction, the mixture was diluted with NaHCO₃ (10 mL) and extracted with EtOAc (10 mL×3). The combined organic layers were washed with brine (10.0 mL), dried over Na₂SO₄, filtered, and concentrated under reduce pressure to give a residue. The residue was purified by silica column chromatography to provide 5,8,8-trimethyl-5-phenyl-5,8,9,10-tetrahydrobenzo[b][1,8]naphthyridin-6(7H)-one (0.075 g, LCMS purity: 100%) as an off-white solid which was further purified by chiral prep HPLC to provide:

Peak 1: (5S)-3-(2,2-dimethylpropyl)-5,8,8-trimethyl-5-phenyl-9,10-dihydro-7H-benzo[b][1,8]naphthyridin-6-one. (5A) (16 mg, 0.0403 mmol, 97.78% purity, 6% yield).

LCMS: (ES⁺) m/z (M+H)⁺=389.0.

¹H NMR (400 MHz, Methanol-d4) δ 7.73 (d, J=2.1 Hz, 1H), 7.40 (d, J=7.6 Hz, 2H), 7.24 (t, J=7.7 Hz, 2H), 7.08 (t, J=7.3 Hz, 1H), 6.84 (d, J=2.2 Hz, 1H), 2.57 (d, J=2.8 Hz, 2H), 2.26 (d, J=3.5 Hz, 2H), 2.17-2.07 (m, 2H), 1.96 (s, 3H), 1.12 (d, J=3.7 Hz, 6H), 0.71 (s, 9H).

Peak 2: (5R)-3-(2,2-dimethylpropyl)-5,8,8-trimethyl-5-phenyl-9,10-dihydro-7H-benzo[b][1,8]naphthyridin-6-one. (5B) (10 mg, 0.0242 mmol, 93.92% purity, 4% yield).

LCMS: (ES⁺) m/z (M+H)⁺=389.0.

¹H NMR (400 MHz, Methanol-d4) δ 7.73 (s, 1H), 7.40 (d, J=7.7 Hz, 2H), 7.24 (t, J=7.6 Hz, 2H), 7.08 (t, J=7.4 Hz, 1H), 6.84 (s, 1H), 2.57 (d, J=3.0 Hz, 2H), 2.26 (d, J=3.5 Hz, 2H), 2.18-2.04 (m, 2H), 1.96 (s, 3H), 1.12 (d, J=3.7 Hz, 6H), 0.71 (s, 9H).

### Example 1.11. (5S)-3-fluoro-5,8,8-trimethyl-5-phenyl-9,10-dihydro-7H-benzo[b][1,8]naphthyridin-6-one (5A) and (5R)-3-fluoro-5,8,8-trimethyl-5-phenyl-9,10-dihydro-7H-benzo[b][1,8]naphthyridin-6-one. (5B)

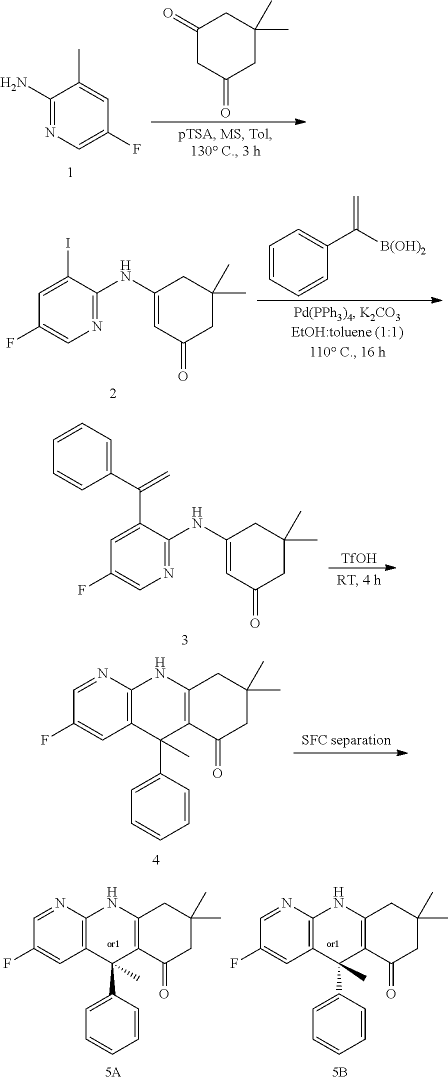

### Step 1: 3-[(5-fluoro-3-iodo-2-pyridyl)amino]-5,5-dimethyl-cyclohex-2-en-1-one. (2)

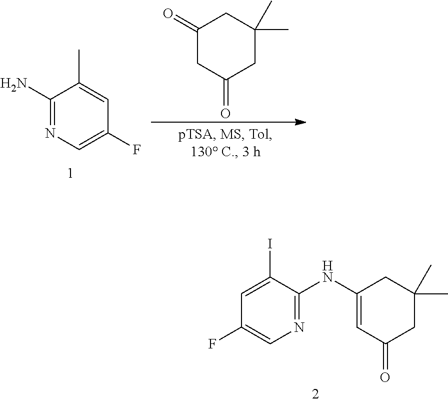

To a stirred solution of 5,5-dimethylcyclohexane-1,3-dione (1767 mg, 12.6 mmol, 1.00 eq) was taken in Toluene (30 mL), then 5-fluoro-3-iodo-pyridin-2-amine (3.00 g, 12.6 mmol, 1.00 eq), PTSA (959 mg, 5.04 mmol, 0.400 eq), and Molecular sieves (500 mg) was added to it and placed the reaction mixture at 130° C. condition for 3 h. Progress of the reaction was monitored by TLC and LCMS. After 3 hours TLC was checked a new polar spot was formed, starting material was fully consumed. The reaction mixture was diluted with water and extracted with EtOAc two times. Combined organic layer were dried over Na₂SO₄ to get crude compound which was advanced to the next step. Crude amount: 3.2 g.

### Step 2: 3-[[5-fluoro-3-(1-phenylvinyl)-2-pyridyl]amino]-5,5-dimethyl-cyclohex-2-en-1-one. (3)

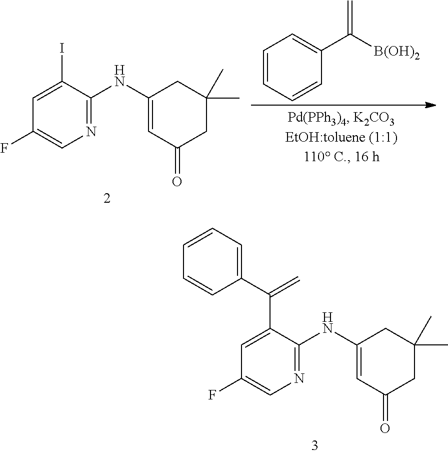

To a stirred solution of 1-phenylvinylboronic acid (111 mg, 0.750 mmol, 0.900 eq) was taken in toluene (4 mL) and EtOH (4 mL) mL), then 3-[(5-fluoro-3-iodo-2-pyridyl)amino]-5,5-dimethyl-cyclohex-2-en-1-one (300 mg, 0.833 mmol, 1.00 eq) and K₂CO₃ (0.24 mL, 1.67 mmol, 2.00 eq) were added to it. Reaction mixture was degassed 10 min. After 10 min Pd(PPh₃)₄ (96 mg, 0.0833 mmol, 0.100 eq) was added to it and degassed for another 10 min and finally the reaction mixture was placed at 110° C. heating condition for 16 h. The reaction mixture was diluted with water and extracted with EtOAc two times. Combined organic layer were dried over Na₂SO₄ to get crude compound. Crude compound was purified using combiflash EtOAc/Hexane as an eluent. Solvent was concentrated under pressure to afford 250 mg. Yield: 89.22%

LCMS: tR=8.57 min., (ES⁺) m/z (M+H)⁺=337.2.

### Example 1.12. (5S)-3-fluoro-5,8,8-trimethyl-5-phenyl-9,10-dihydro-7H-benzo[b][1,8]naphthyridin-6-one (5A)

### (5R)-3-fluoro-5,8,8-trimethyl-5-phenyl-9,10-dihydro-7H-benzo[b][1,8]naphthyridin-6-one. (5B)

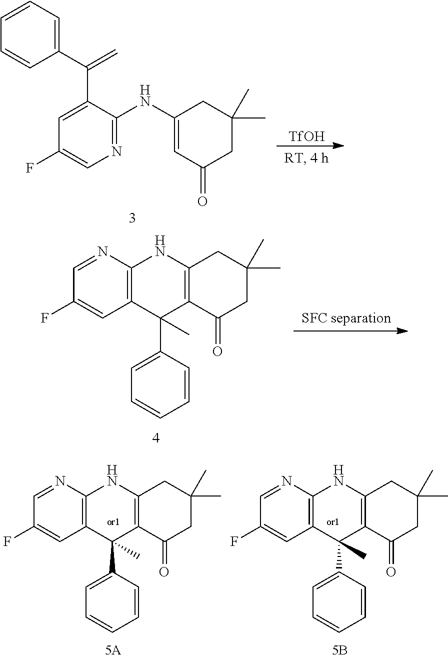

To a stirred solution of 3-[[5-fluoro-3-(1-phenylvinyl)-2-pyridyl]amino]-5,5-dimethyl-cyclohex-2-en-1-one (250 mg, 0.743 mmol, 1.00 eq) was taken, then cooled the reaction mass at 0° C. Triflic acid (0.66 mL, 7.43 mmol, 10.0 eq) was added to it dropwise and placed the reaction mixture at RT condition for 4 hours. Progress of the reaction was monitored by LCMS. After 2 hours TLC was checked a new polar spot was formed, starting material was fully consumed. After 4 h, the reaction mixture was quenched with NaHCO₃ solution (20 mL) and extracted with EtOAc (3×30 mL). The combined organic layer was washed with brine solution (20 mL), dried over anhydrous Na₂SO₄, and was concentrated under reduced pressure. The crude was purified using combiflash EtOAc/Hex as an eluent. Solvent was concentrated under pressure to afford 183 mg pure compound.

The two enantiomers were separated by chiral preparatory SFC (Column: CHIRALPAK AS-H (21 mm×250 mm), 5μ Flow: 60 g/min Mobile Phase: 75% CO₂+25% (MEOH) ABPR: 110 bar; Temp: 35° C.; UV: 240 nm; diluent: Methanol) to give:

Peak 1: (5S)-3-fluoro-5,8,8-trimethyl-5-phenyl-9,10-dihydro-7H-benzo[b][1,8]naphthyridin-6-one (5A), (59 mg, 0.172 mmol, 98.14% purity, 23% yield).

LCMS: tR=2.92 min., (ES⁺) m/z (M+H)⁺=337.2.

HPLC: (purity: 98.14%).

¹H NMR (400 MHz, DMSO-d₆) δ=1.01 (s, 6H), 1.94 (s, 3H), 1.97 (s, 1H), 2.02 (s, 1H), 2.07 (s, 1H), 2.50 (s, 1H), 6.98 (d, 1H), 7.01 (m, 1H), 7.24 (m, 2H), 7.33 (m, 2H), 7.96 (d, 1H), 9.87 (s, 1H).

Peak 2: (5R)-3-fluoro-5,8,8-trimethyl-5-phenyl-9,10-dihydro-7H-benzo[b][1,8]naphthyridin-6-one (5B), (59 mg, 0.170 mmol, 97.27% purity, 23% yield).

LCMS: tR=2.91 min., (ES⁺) m/z (M+H)⁺=337.2.

HPLC: (purity: 97.27%).

¹H NMR (400 MHz, DMSO-d₆) δ=1.01 (s, 6H), 1.94 (s, 3H), 1.97 (s, 1H), 2.02 (s, 1H), 2.07 (s, 1H), 2.50 (s, 1H), 6.98 (d, 1H), 7.01 (m, 1H), 7.24 (m, 2H), 7.33 (m, 2H), 7.96 (d, 1H), 9.87 (s, 1H).

### Example 1.13. Compound 6A

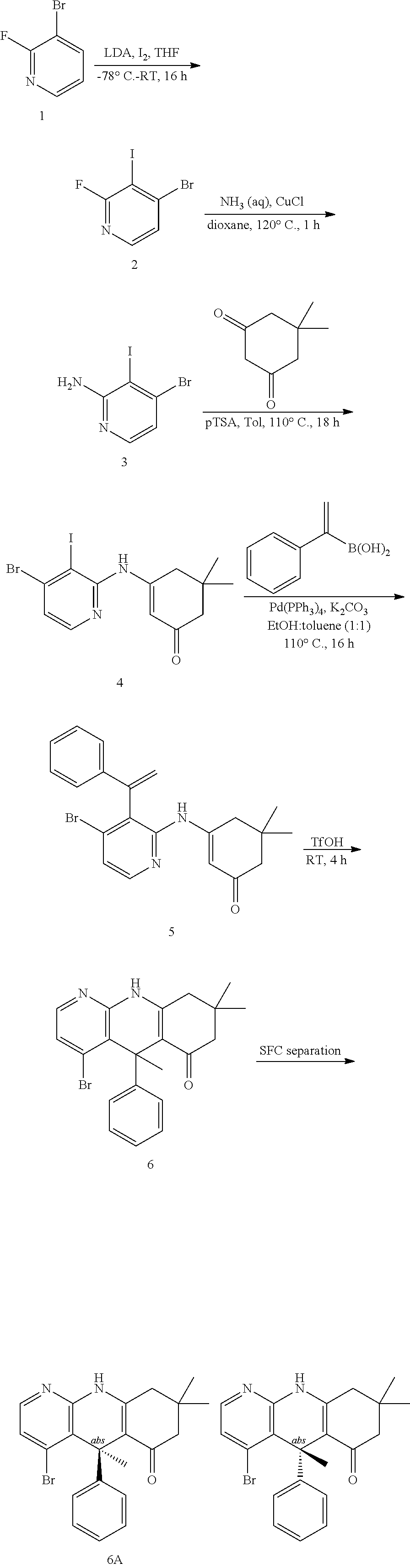

### Step 1: 4-bromo-2-fluoro-3-iodo-pyridine. (2)

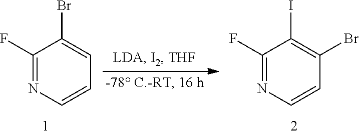 [SMILES: Fc1ncccc1Br ; Fc1nccc(Br)c1I]

To a flame-dried 2-neck flask under argon were added, Lithium diisopropylamide (33 mL, 31.3 mmol, 1.10 eq) (2.0 M solution in THF) and THF (10 mL), and the resulting solution was cooled to −78° C. in a dry ice/acetone bath. A solution of 3-bromo-2-fluoro-pyridine (5.00 g, 28.4 mmol, 1.00 eq) in THF (20 mL) was added drop wise to the reaction mixture over 0.5 h. The internal temperature of the reaction was monitored and maintained below −65° C. throughout the addition. The reaction mixture was stirred for an additional 1 h at −70° C. A solution of iodine (2.30 g, 28.4 mmol, 1.00 eq) in THF (5 mL) was added drop wise to the reaction mixture over 1 h, and the internal temperature of the reaction was again maintained below −65° C. throughout the addition. The reaction mixture was stirred for 30 min at −70° C. Reaction Mixture was quenched with of brine (10 mL). The quenched reaction mixture was warmed to room temperature and extracted with EtOAc (2×50 mL). The combined organic extracts were washed successively with saturated aqueous NaHCO₃ solution, then brine, and dried over Na₂SO₄ then filtered. The filtrate was concentrated, and the residue purified by column chromatography using EtOAc and Hexane as the eluent to afford title compound (4.1 g, 48%).

### Step 2: 4-bromo-3-iodo-pyridin-2-amine. (3)

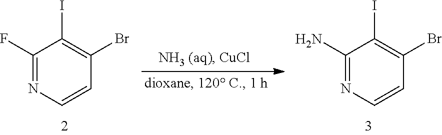 [SMILES: Fc1nccc(Br)c1I ; Nc1nccc(Br)c1I]

To a stirred solution of 4-bromo-2-fluoro-3-iodo-pyridine (1.20 g, 3.98 mmol, 1.00 eq), Ammonium hydroxide solution 28% NH₃ in H₂O, in 1,4-dioxane (10 mL) Copper(I) chloride (39 mg, 0.398 mmol, 0.1000 eq) was added at RT then the tube was sealed with a screw stopper and then the reaction mixture was heated at 110° C. for 18 h. The reaction was monitored by TLC and LCMS. After completion of the starting material as indicated by TLC, then the reaction mixture was concentrated under reduced pressure and reaction mixture was extracted with ethyl acetate, the combined organic layer was washed with brine and dried over anhydrous Na₂SO₄. After filtration and removal of solvent in vacuo, the crude product was purified by column chromatography using EtOAc and Hexane as the eluent to afford the title compound in 85% yield (1.000 g).

### Step 3: 3-[(4-bromo-3-iodo-2-pyridyl)amino]-5,5-dimethyl-cyclohex-2-en-1-one. (4)

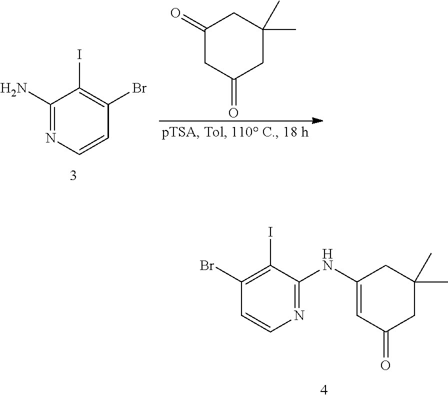

To a stirred solution of 5,5-dimethylcyclohexane-1,3-dione (1.64 g, 11.7 mmol, 1.00 eq), 4-bromo-3-iodo-pyridin-2-amine (3.50 g, 11.7 mmol, 1.00 eq) in Toluene (30 mL), p-Toluene sulfonic acid (1.34 g, 7.03 mmol, 0.600 eq) was added and then the reaction mixture was heated at 130° C. for 18 h. Reaction monitored by TLC and LCMS. After completion of the starting material, the reaction was quenched with NaHCO₃ sol. Then the reaction mixture was washed with water (50 ml) and brine (20 ml), dried over Na₂SO₄ then filtered. The filtrate was concentrated, and the residue was purified by column chromatography using EtOAc and Hexane as the eluent to afford 3-[(4-bromo-3-iodo-2-pyridyl)amino]-5,5-dimethyl-cyclohex-2-en-1-one (3.8 g, 77%).

### Step 4: 3-[[4-bromo-3-(1-phenylvinyl)-2-pyridyl]amino]-5,5-dimethyl-cyclohex-2-en-1-one. (5)

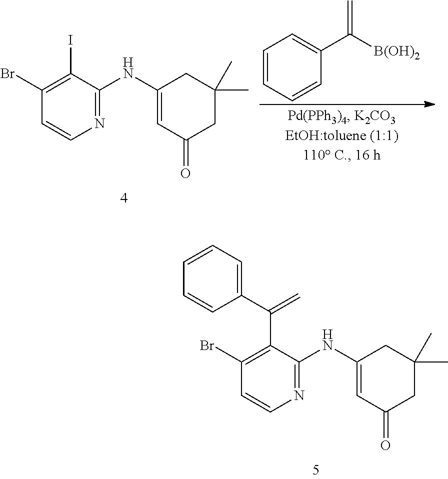

To a stirred solution of 3-[(4-bromo-3-iodo-2-pyridyl)amino]-5,5-dimethyl-cyclohex-2-en-1-one (200 mg, 0.475 mmol, 1.00 eq), 1-phenylvinylboronic acid (63 mg, 0.427 mmol, 0.900 eq), K₂CO₃ (131 mg, 0.950 mmol, 2.00 eq), in EtOH (4 mL) and Toluene (4 mL), the reaction suspension was bubbled with N₂ for 5 min then Tetrakis(triphenylphosphine)palladium(0) (55 mg, 0.0475 mmol, 0.100 eq) was added (U/N₂) and then the reaction mixture was heated at 110° C. for 14 hrs. The reaction was monitored by TLC and LCMS. After completion of the starting material as indicated by TLC, then the reaction mixture was concentrated under reduced pressure and mixture was extracted with ethyl acetate, the combined organic layer was washed with brine and dried over anhydrous Na₂SO₄. After filtration and removal of solvent in vacuo, the crude product was purified by column chromatography using EtOAc and Hexane as the eluent to afford 3-[[4-bromo-3-(1-phenylvinyl)-2-pyridyl]amino]-5,5-dimethyl-cyclohex-2-en-1-one (148 mg, 78%).

### Step 5: (5S)-4-bromo-5,8,8-trimethyl-5-phenyl-9,10-dihydro-7H-benzo[b][1,8]naphthyridin-6-one (6A)

### (5R)-4-bromo-5,8,8-trimethyl-5-phenyl-9,10-dihydro-7H-benzo[b][1,8]naphthyridin-6-one (6B)

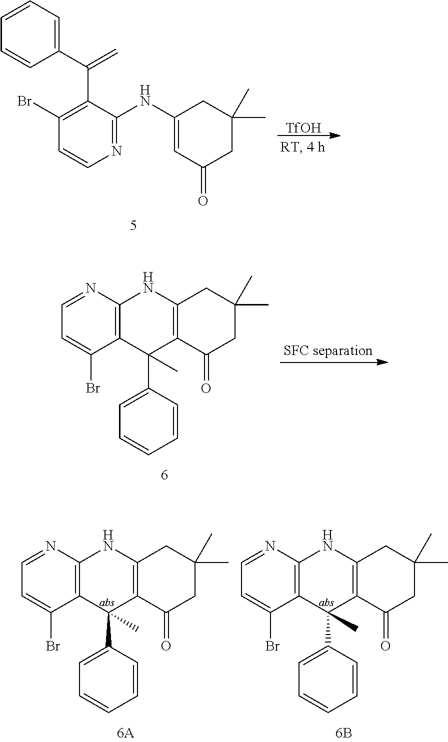

3-[[4-bromo-3-(1-phenylvinyl)-2-pyridyl]amino]-5,5-dimethyl-cyclohex-2-en-1-one (100 mg, 0.252 mmol, 1.00 eq) was dissolved in trifluoromethanesulfonic acid (1.5 mL, 17.0 mmol, 67.5 eq). The reaction mixture was stirred at 25° C. for 18 h. Reaction monitored by TLC and LCMS, showed starting material was consumed. The reaction mixture was diluted with water and extracted with EtOAc (50 ml×2). The combined organic layer was washed with washed with NaHCO₃ (5 ml) and brine (10 ml) and dried over Na₂SO₄, filtered, and concentrated under reduced pressure to give residue. The residue was purified by column chromatography (EtOAc and Hexane).

Enantiomers separated by Chiral SFC (Column (R,R) WHELK-O1 (21 mm×250 mm), 5μ Flow: 50 g/min Mobile Phase: 70% CO₂+30% (0.3% IPamine in Methanol) ABPR: 100 bar, Temp: 35° C. UV: 220 nm diluent: MEOH+DCM to obtain:

Peak 1: (5S)-4-bromo-5,8,8-trimethyl-5-phenyl-9,10-dihydro-7H-benzo[b][1,8]naphthyridin-6-one (6A) (24 mg, 24%).

Peak 2: (5R)-4-bromo-5,8,8-trimethyl-5-phenyl-9,10-dihydro-7H-benzo[b][1,8]naphthyridin-6-one (6B) (18 mg, 18%).

### Example 1.14. (5S)-4,5,8,8-tetramethyl-5-phenyl-9,10-dihydro-7H-benzo[b][1,8]naphthyridin-6-one. (7)

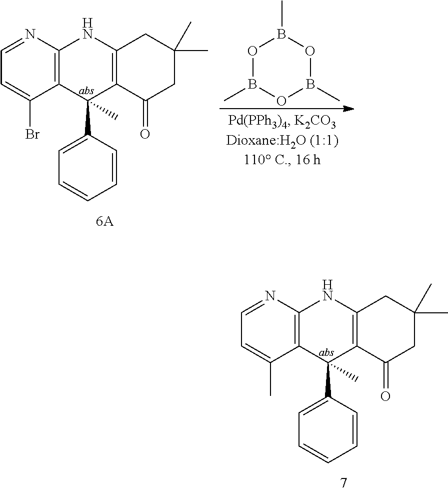

To a stirred solution of (5R)-4-bromo-5,8,8-trimethyl-5-phenyl-9,10-dihydro-7H-benzo[b][1,8]naphthyridin-6-one (150 mg, 0.378 mmol, 1.00 eq) in 1,4-dioxane (3 mL) were added 2,4,6-trimethyl-1,3,5,2,4,6-trioxatriborinane (47 mg, 0.378 mmol, 1.00 eq) and K₂CO₃ (156 mg, 1.13 mmol, 3.00 eq) degassed with Ar for 5 min, after that added tetrakis(triphenylphosphine)palladium (44 mg, 0.0378 mmol, 0.1000 eq) again degassed for 5-10 min, resulting mixture was reflux at 100° C. for 16 h. After 16 h reaction was monitored by TLC or LCMS. TLC with 5% MeOH in DCM showed formation of desired product. Mixture was filter through cartridge filtrate diluted with water and aqueous layer was extracted with ethyl acetate combined organic layer was dried over Na₂SO₄ and concentrated under reduced pressure to give crude compound. Crude residue was purified by prep HPLC (Column: YMC-Actus Triart C18 (250×20 mm, 5μ) flow rate of 16.0 ml/min. Mobile phase: A=20 mM NH₄HCO₃ in water, B=Acetonitrile) to give (5S)-4,5,8,8-tetramethyl-5-phenyl-9,10-dihydro-7H-benzo[b][1,8]naphthyridin-6-one, (30 mg, 0.0885 mmol, 99.52% purity, 23% yield) as a white solid.

LCMS: tR=1.88 min., (ES⁺) m/z (M+H)⁺=333.31.

HPLC: (purity: 99.52%).

¹H NMR (400 MHz, DMSO-d6) δ=0.891 (s, 3H) 0.973 (s, 3H) 1.601 (s, 3H) 1.844 (s, 1H) 1.986 (s, 4H) 2.499 (s, 2H) 6.584 (d, J=4.92 Hz; 1H) 7.051 (t, J=7.2 Hz; 1H) 7.193 (t, J=1.88 Hz; 2H) 7.332 (d, J=7.6 Hz; 1H) 7.879 (d, J=4.84 Hz; 1H) 9.634 (s, 1H).

### Example 1.15. (5S)-4-ethyl-5,8,8-trimethyl-5-phenyl-9,10-dihydro-7H-benzo[b][1,8]naphthyridin-6-one. (9)

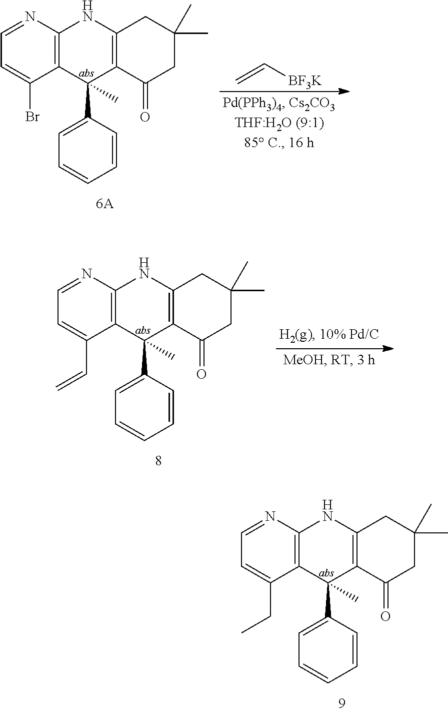

### Step 1. (5S)-5,8,8-trimethyl-5-phenyl-4-vinyl-9,10-dihydro-7H-benzo[b][1,8]naphthyridin-6-one. (8)

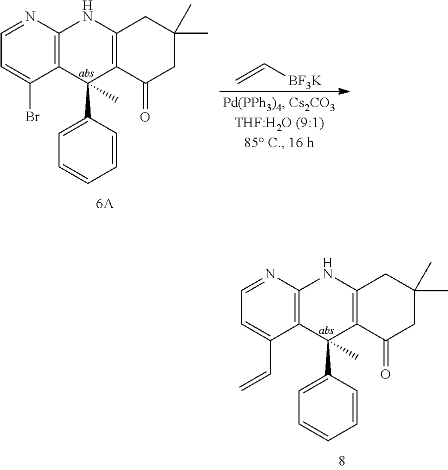

To a stirred solution of (5R)-4-bromo-5,8,8-trimethyl-5-phenyl-9,10-dihydro-7H-benzo[b][1,8]naphthyridin-6-one (140 mg, 0.352 mmol, 1.00 eq) in tetrahydrofuran (3 mL) and water (0.30 mL) were added potassium vinyl trifluoroborate (94 mg, 0.705 mmol, 2.00 eq) Cs₂CO₃ (0.4 mL, 0.881 mmol, 2.50 eq) degassed with Ar for 5 min added tetrakis(triphenylphosphine)palladium (41 mg, 0.0352 mmol, 0.100 eq) degassed with Ar 5 min, resulting mixture was heated at 85° C. for 16 h. After 16 h reaction was monitored by TLC or LCMS. TLC: 30% EA in hexane shows formation of desired product. Mixture was filtered through cartridge diluted with water and extracted with EtOAc combined organic layer was dried over Na₂SO₄ and concentrated under reduced pressure to get crude compound. Crude residue was purified by prep HPLC (Column: HYDROSPHERE C18 (250×20 mm, 5μ) flow rate=16.0 ml/min. Mobile phase: A=10 mM NH₄OAC in water, B=Acetonitrile) to get (5S)-5,8,8-trimethyl-5-phenyl-4-vinyl-9,10-dihydro-7H-benzo[b][1,8]naphthyridin-6-one, (9.7 mg, 0.0280 mmol, 99.62% purity, 8% yield) as a white solid.

LCMS: tR=2.14 min., (ES⁺) m/z (M+H)⁺=345.2.

¹H NMR (400 MHz, DMSO-d6) δ=0.874 (s, 3H) 0.972 (s, 3H) 1.837 (d, 1H) 1.992 (d, 1H) 2.071 (s, 3H) 5.077 (d, 1H) 5.532 (d, 1H) 6.292 (t, 1H) 6.876 (d, 1H) 7.061 (t, 1H) 7.211 (t, 2H) 7.354 (d, 2H) 8.008 (d, 1H) 9.724 (s, 1H).

### (5S)-4-ethyl-5,8,8-trimethyl-5-phenyl-9,10-dihydro-7H-benzo[b][1,8]naphthyridin-6-one. (9)

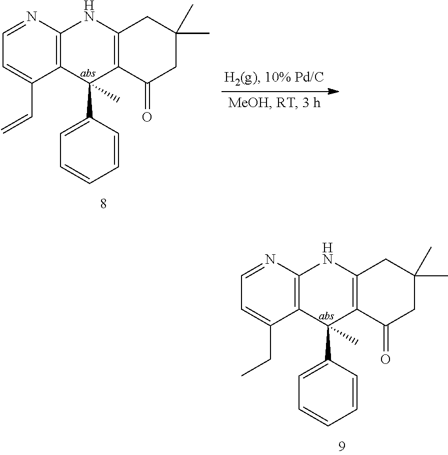 [SMILES: C=Cc1ccnc2c1[C@](C)(c1ccccc1)C1=C(CC(C)(C)CC1=O)N2 ; CCc1ccnc2c1[C@](C)(c1ccccc1)C1=C(CC(C)(C)CC1=O)N2]

To a round bottom flask (5S)-5,8,8-trimethyl-5-phenyl-4-vinyl-9,10-dihydro-7H-benzo[b][1,8]naphthyridin-6-one (25 mg, 0.0726 mmol, 1.00 eq) was taken in methanol (5 mL) then added 10% Pd—C (23 mg, 0.218 mmol, 3.00 eq) and stirred the reaction mixture in presence of hydrogen atm. at 25° C. for 12 h. Reaction was monitored by TLC and LCMS. TLC: 50% EtOAc in Hexane showed the formation of desired product. After completion of reaction, the reaction mixture was passed through celite, and crude was concentrated under reduced pressure. The crude was purified by column chromatography, eluting with 40% ethyl acetate in hexane to afford (5S)-4-ethyl-5,8,8-trimethyl-5-phenyl-9,10-dihydro-7H-benzo[b][1,8]naphthyridin-6-one, (14 mg, 0.0415 mmol, 99.27% purity, 57% yield).

LCMS: tR=2.91 min., (ES⁺) m/z (M+H)⁺=347.3.

HPLC: (purity: 99.27%).

¹H NMR (400 MHz, DMSO-d6) δ=0.37 (t, 3H), 0.89 (s, 3H), 0.97 (s, 3H), 1.83 (d, 1H), 1.96 (d, 1H), 1.99 (s, 3H), 2.18 (q, 2H), 2.39 (s, 2H), 6.68 (d, 1H), 7.02 (t, 1H), 7.18 (t, 2H), 7.3518 (d, 2H), 7.92 (d, 1H).

### Example 1.16. (5S) 4-isopropyl-5,8,8-trimethyl-5-phenyl-9,10-dihydro-7H-benzo[b][1,8]naphthyridin-6-one. (11)

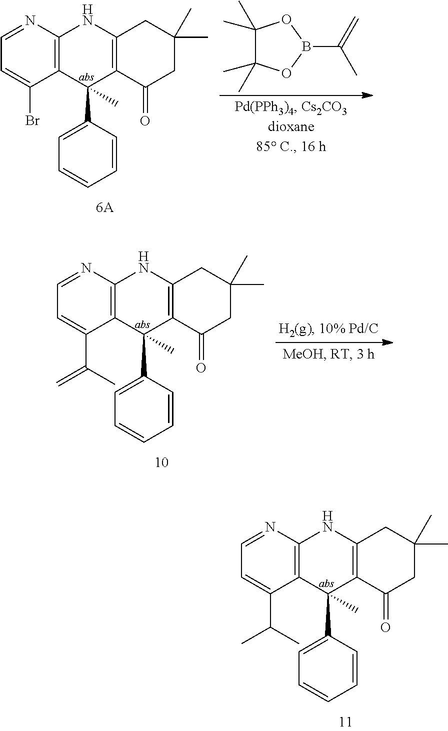

### Step 1: (5S)-4-isopropenyl-5,8,8-trimethyl-5-phenyl-9,10-dihydro-7H-benzo[b][1,8]naphthyridin-6-one. (10)

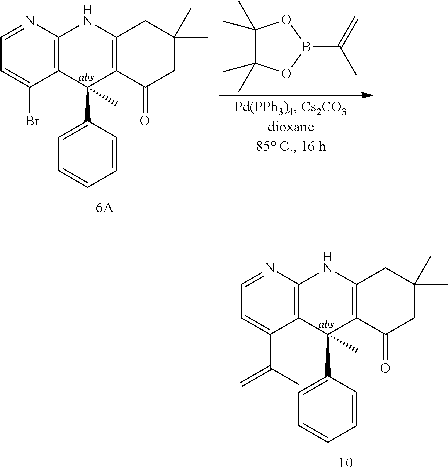

To a round bottom flask, (5R)-4-bromo-5,8,8-trimethyl-5-phenyl-9,10-dihydro-7H-benzo[b][1,8]naphthyridin-6-one (300 mg, 0.755 mmol, 1.00 eq) was taken in 1,4-dioxane (10 mL) then Pd(PPH₃)₄ (87 mg, 0.0755 mmol, 0.100 eq) was added to this reaction mixture at OC. Then 2-isopropenyl-4,4,5,5-tetramethyl-1,3,2-dioxaborolane (127 mg, 0.755 mmol, 1.00 eq) was added to this reaction mixture and stirred the reaction mixture under N₂ atm for 16 h at 85° C. The reaction was monitored by TLC and LCMS. After completion of the starting material as indicated by TLC, then the reaction mixture was concentrated under reduced pressure, and mixture was extracted with ethyl acetate, the combined organic layer was washed with brine and dried over anhydrous Na₂SO₄. After filtration and removal of solvent in vacuo, the crude product was purified by column chromatography using EtOAc and Hexane as the eluent. TLC (Rf=0.7), 30% EtOAc/Hexane. Synthesized compound confirmed by LCMS.

### (5S)-4-isopropyl-5,8,8-trimethyl-5-phenyl-9,10-dihydro-7H-benzo[b][1,8]naphthyridin-6-one. (11)

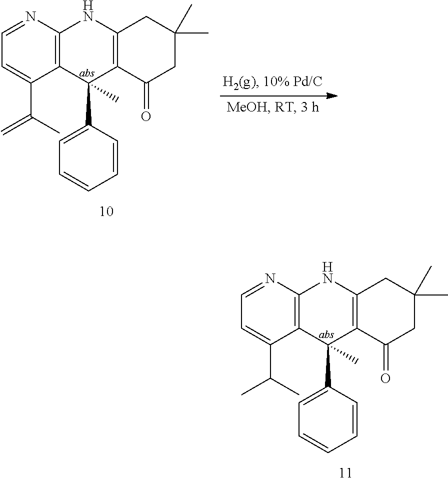 [SMILES: C=C(C)c1ccnc2c1[C@](C)(c1ccccc1)C1=C(CC(C)(C)CC1=O)N2 ; CC(C)c1ccnc2c1[C@](C)(c1ccccc1)C1=C(CC(C)(C)CC1=O)N2]

To a round bottom flask (5S)-4-isopropenyl-5,8,8-trimethyl-5-phenyl-9,10-dihydro-7H-benzo[b][1,8]naphthyridin-6-one (9.0 mg, 0.0251 mmol, 1.00 eq) was taken in methanol (5 mL) then added 10% Pd/C (0.01 mL, 0.100 mmol, 4.00 eq) and stirred the reaction mixture in presence of hydrogen atm. at 25° C. for 12 h. Reaction was monitored by TLC and LCMS. TLC: 50% EtOAc in Hexane showed the formation of desired product. After completion of reaction, the reaction mixture was passed through celite, and crude was concentrated under reduced pressure. The crude was purified by column chromatography, eluting with 40% ethyl acetate in hexane to afford (5S)-4-isopropyl-5,8,8-trimethyl-5-phenyl-9,10-dihydro-7H-benzo[b][1,8]naphthyridin-6-one, (3.0 mg, 0.00743 mmol, 88.64% purity, 30% yield).

LCMS: tR=5.83 min., (ES⁺) m/z (M+H)⁺=361.33.

HPLC: (purity: 88.64%).

¹H NMR (400 MHz, DMSO-d6) δ=0.16 (d, 3H), 0.8787 (s, 3H), 0.97 (s, 3H), 1.03 (d, 6H), 1.83 (d, 1H), 1.97 (d, 1H), 2.02 (s, 3H), 2.39 (d, 2H), 2.93 (m, 1H), 6.80 (d, 1H), 7.03 (t, 1H), 7.17 (t, 2H), 7.37 (bs, 2H), 7.96 (d, 1H), 9.64 (s, 1H).

### Example 1.17. (5S)-4-(2,2-dimethylpropyl)-5,8,8-trimethyl-5-phenyl-9,10-dihydro-7H-benzo[b][1,8]naphthyridin-6-one (6A)

### (5R)-4-(2,2-dimethylpropyl)-5,8,8-trimethyl-5-phenyl-9,10-dihydro-7H-benzo[b][1,8]naphthyridin-6-one (6B)

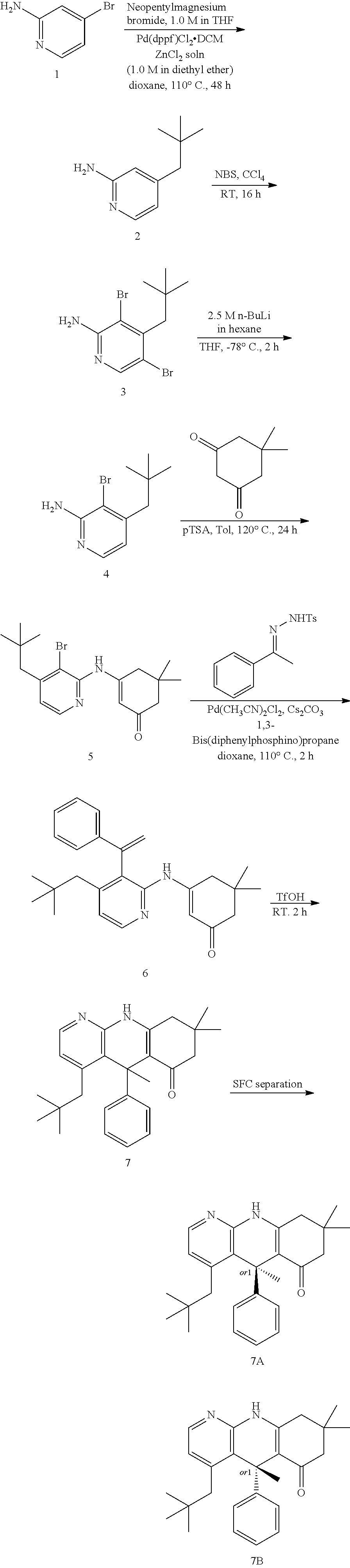

### Step 1: 4-(2,2-dimethylpropyl)pyridin-2-amine. (2)

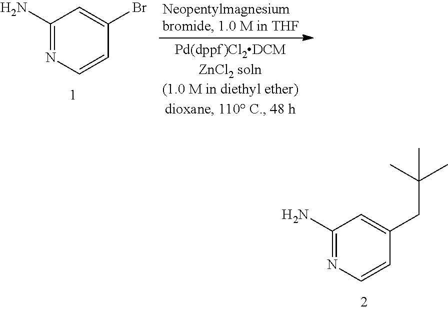

Neopentylmagnesium bromide, 1.0 M in THF (1 M in, 23.12 mL, 23.1 mmol, 4.00 eq) was added to a solution of Zinc chloride solution (1.0 M in diethyl ether) (1 M in, 11.56 mL, 11.6 mmol, 2.00 eq) under argon at 0° C. The solution was diluted with 1,4-dioxane (10 mL) and transferred into a suspension of 4-bromopyridin-2-amine (1.00 g, 5.78 mmol, 1.00 eq) and Pd(dppf)Cl₂·DCM (0.094 g, 0.116 mmol, 0.0200 eq) in 1,4-dioxane (5 mL). The mixture was heated at 110° C. for 48 h. After cooling to room temperature, the mixture was poured into water (50 mL) and aqueous NaHCO₃ (1 M; 40 mL) was added. The mixture was extracted with EtOAc (3×50 mL). The combined organic phases were washed with brine, dried over Na₂SO₄, and concentrated to give crude product which was purified by silica gel chromatography using 20% EtOAc in hexane to provide 4-(2,2-dimethylpropyl)pyridin-2-amine, (800 mg, 4.33 mmol, 89% purity, 75% yield).

LCMS: (ES⁺) m/z (M+H)⁺=165.5.

¹H NMR (400 MHz, DMSO-d6) δ=7.78 (d, J=5.1 Hz, 1H), 6.28 (d, J=5.2 Hz, 1H), 6.21 (s, 1H), 5.75 (s, 2H), 2.30 (s, 2H), 0.89 (s, 9H).

### Step 2: 3,5-dibromo-4-(2,2-dimethylpropyl)pyridin-2-amine. (3)

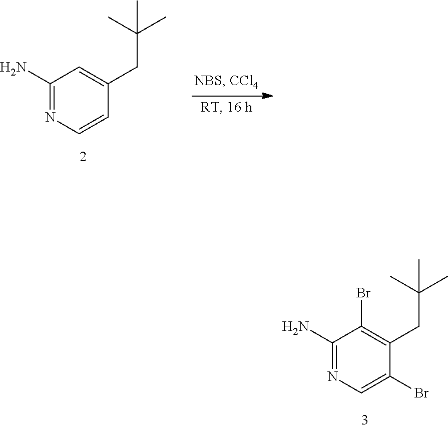

To a solution of 4-(2,2-dimethylpropyl)pyridin-2-amine (800 mg, 4.87 mmol, 1.00 eq) in carbon tetrachloride (20 mL) was added N-Bromosuccinimide (1907 mg, 10.7 mmol, 2.20 eq) at 25° C. After the mixture was stirred at 25° C. for 16 h, it was poured into water (50 mL), extracted with DCM (100 mL×3). The combined organic layers was washed with brine (50 mL), dried over anhydrous Na2SO4, concentrated and purified by silica gel column chromatography using 8% EtOAc in Hexane to give 3,5-dibromo-4-(2,2-dimethylpropyl)pyridin-2-amine, (700 mg, 2.13 mmol, 97.92% purity, 44% yield). Analytical data is consistent with desired structure.

LCMS: (ES⁺) m/z [M+H]⁺⁴=325.0.

### Step 3: 3-bromo-4-(2,2-dimethylpropyl)pyridine-2-amine. (4)

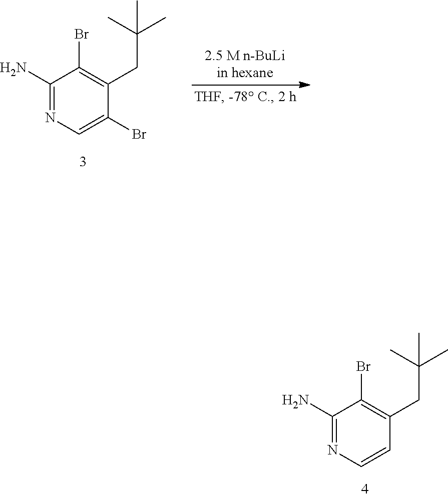

Into a 100-mL 3-necked round-bottom flask purged and maintained with an inert atmosphere of nitrogen was placed 3,5-dibromo-4-(2,2-dimethylpropyl)pyridin-2-amine (0.70 g, 2.17 mmol, 1.00 eq) in tetrahydrofuran (14 mL) To this was added a solution of 2.5 M n-BuLi in hexane (2.5 M in, 2.78 mL, 6.96 mmol, 3.20 eq) at −78° C. The resulting solution was stirred at −78° C. for 2 h, quenched by the addition of 25 mL of saturated aqueous NH₄Cl and then extracted with 2×50 mL of ethyl acetate. The combined organic layers were washed with brine, dried over anhydrous Na₂SO₄, and concentrated under vacuum. The crude product was purified by silica column chromatography using 22% EtOAc in hexanes to afford 3-bromo-4-(2,2-dimethylpropyl)pyridine-2-amine, (375 mg, 1.54 mmol, 100% purity, 71% yield) as light-yellow solid. Analytical data is consistent with structure.

LCMS: (ES⁺) m/z (M+H)⁺=245.1.

### Step 4: 3-[[3-bromo-4-(2,2-dimethylpropyl)-2-pyridyl]amino]-5,5-dimethyl-cyclohex-2-en-1-one. (5)

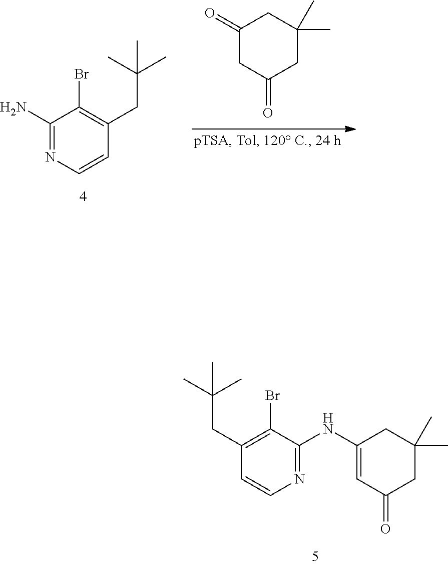

A solution of 3-bromo-4-(2,2-dimethylpropyl)pyridin-2-amine (0.35 g, 1.44 mmol, 1.00 eq)₃-bromo-4-(2,2-dimethylpropyl)pyridin-2-amine (0.35 g, 1.44 mmol, 1.00 eq), 5,5-dimethylcyclohexane-1,3-dione (0.20 g, 1.44 mmol, 1.00 eq) and pTSA (0.074 g, 0.432 mmol, 0.300 eq) in toluene (2.4905 mL) was stirred at 120° C. for 24 h. Progress of reaction was monitored by TLC using Ethyl acetate:Hexanes (2:8). After completion of the rection, ethyl acetate was added, and organic layer was washed with saturated NaHCO₃ solution (100 mL×2), water (100 mL) and brine (100 mL), dried over anhydrous Na₂SO₄ and concentrated under vacuum to provide crude product, which was purified by silica gel chromatography using 100% ethyl acetate to afford 3-[[3-bromo-4-(2,2-dimethylpropyl)-2-pyridyl]amino]-5,5-dimethyl-cyclohex-2-en-1-one, (250 mg, 0.684 mmol, 100% purity, 48% yield) as a light yellow solid. Analytical data is consistent with structure.

LCMS: (ES⁺) m/z [M+H]⁺²=367.7.

### Step 5: 3-[[4-(2,2-dimethylpropyl)-3-(1-phenylvinyl)-2-pyridyl]amino]-5,5-dimethyl-cyclohex-2-en-1-one. (6)

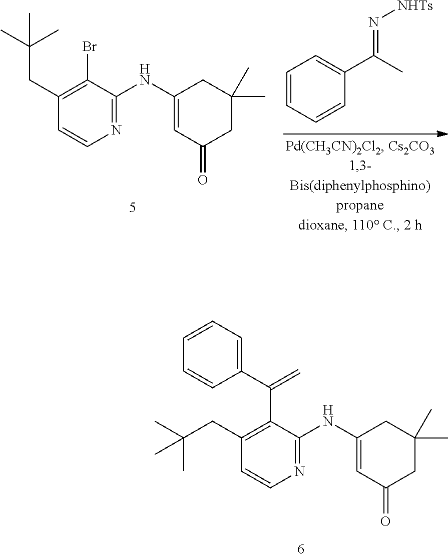

3-[[3-bromo-4-(2,2-dimethylpropyl)-2-pyridyl]amino]-5,5-dimethyl-cyclohex-2-en-1-one (0.25 g, 0.684 mmol, 1.00 eq), 4-methyl-N-[(E)-1-phenylethylideneamino]benzenesulfonamide (0.30 g, 1.03 mmol, 1.50 eq) and Cesium carbonate (0.45 g, 1.37 mmol, 2.00 eq) were added into 1,4-dioxane (2.0612 mL) The reaction suspension was bubbled with argon for 10 min, then Pd(CH3CN)2Cl2 (0.018 g, 0.0684 mmol, 0.1000 eq) and 1,3-Bis(diphenylphosphino)propane (0.028 g, 0.0684 mmol, 0.1000 eq) were added. The reaction mixture was stirred at 110° C. for 2 h under nitrogen atmosphere. Reaction was monitored by TLC using ethyl acetate:hexanes (4:6). After completion of the reaction, the reaction mixture was concentrated and extracted with ethyl acetate (3×25 mL). The combined organic layer was dried over Na₂SO₄ and concentrated to provide crude product, which was purified by flash silica gel chromatography (Hexane/EtOAc=1/1) to give 3-[[4-(2,2-dimethylpropyl)-3-(I-phenylvinyl)-2-pyridyl]amino]-5,5-dimethyl-cyclohex-2-en-1-one, (130 mg, 0.298 mmol, 89% purity, 44% yield) as a light orange solid. LCMS is consistent with desired product.

LCMS: (ES⁺) m/z (M+H)⁺=388.2.

### Example 1.18. (5S)-4-(2,2-dimethylpropyl)-5,8,8-trimethyl-5-phenyl-9,10-dihydro-7H-benzo[b][1,8]naphthyridin-6-one. (7A)

### (5R)-4-(2,2-dimethylpropyl)-5,8,8-trimethyl-5-phenyl-9,10-dihydro-7H-benzo[b][1,8]naphthyridin-6-one. (7B)

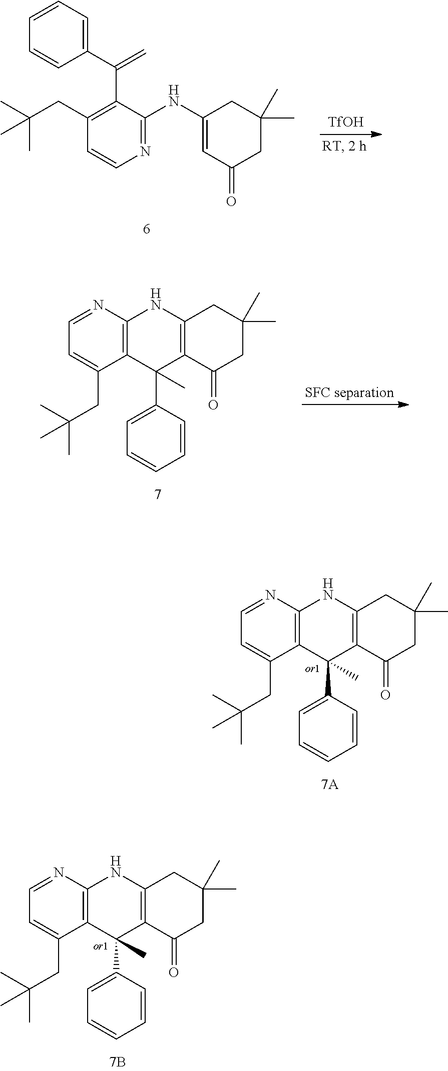

3-[[4-(2,2-dimethylpropyl)-3-(1-phenylvinyl)-2-pyridyl]amino]-5,5-dimethyl-cyclohex-2-en-1-one (0.13 g, 0.335 mmol, 1.00 eq) was dissolved in Trifluoromethanesulfonic acid (0.12 mL, 1.34 mmol, 4.01 eq). The resulting mixture was stirred at 25° C. for 2 h. Reaction progress was monitored by TLC EtOAc:Hexanes (1:1). After completion of reaction, the mixture was diluted with NaHCO3 (10 mL) and extracted with EtOAc (10 mL×3). The combined organic layers were washed with brine (10.0 mL), dried over Na₂SO₄, filtered, and concentrated under reduced pressure to give a residue. The residue was purified by silica column chromatography to provide 5,8,8-trimethyl-5-phenyl-5,8,9,10-tetrahydrobenzo[b][1,8]naphthyridin-6(7H)-one (0.16 g, LCMS purity: 100%) as an off white solid.

The two enantiomers were separated by chiral preparatory SFC:

Peak 1: (5S)-4-(2,2-dimethylpropyl)-5,8,8-trimethyl-5-phenyl-9,10-dihydro-7H-benzo[b][1,8]naphthyridin-6-one. (7A) (7.0 mg, 0.0170 mmol, 94.44% purity, 5% yield).

LCMS: (ES⁺) m/z (M+H)⁺=389.0.

¹H NMR (400 MHz, Methanol-d4) δ 7.94 (d, J=5.3 Hz, 1H), 7.45 (d, J=7.6 Hz, 2H), 7.21 (t, J=7.6 Hz, 2H), 7.05 (dd, J=23.1, 6.3 Hz, 2H), 2.62 (d, J=15.0 Hz, 1H), 2.53-2.40 (m, 2H), 2.30 (d, J=15.0 Hz, 11H), 2.10 (s, 3H), 1.93 (d, J=16.1 Hz, 11H), 1.08 (s, 3H), 0.99 (s, 3H), 0.61 (s, 9H).

Peak 2: (5R)-4-(2,2-dimethylpropyl)-5,8,8-trimethyl-5-phenyl-9,10-dihydro-7H-benzo[b][1,8]naphthyridin-6-one. (7B) (10 mg, 0.0246 mmol, 95.64% purity, 7% yield).

LCMS: (ES⁺) m/z (M+H)⁺=389.0.

¹H NMR (400 MHz, Methanol-d4) δ=7.93 (d, J=5.3 Hz, 1H), 7.45 (d, J=7.7 Hz, 2H), 7.21 (t, J=7.7 Hz, 2H), 7.05 (dd, J=22.9, 6.3 Hz, 2H), 2.62 (d, J=15.0 Hz, 1H), 2.54-2.41 (m, 2H), 2.30 (d, J=15.0 Hz, 1H), 2.10 (s, 3H), 2.06 (s, 1H), 1.93 (d, J=16.1 Hz, 1H), 1.08 (s, 3H), 0.99 (s, 3H), 0.61 (s, 9H).

### Example 1.19. (5S)-4-fluoro-5,8,8-trimethyl-5-phenyl-9,10-dihydro-7H-benzo[b][1,8]naphthyridin-6-one. (5A)

### (5R)-4-fluoro-5,8,8-trimethyl-5-phenyl-9,10-dihydro-7H-benzo[b][1,8]naphthyridin-6-one. (5B)

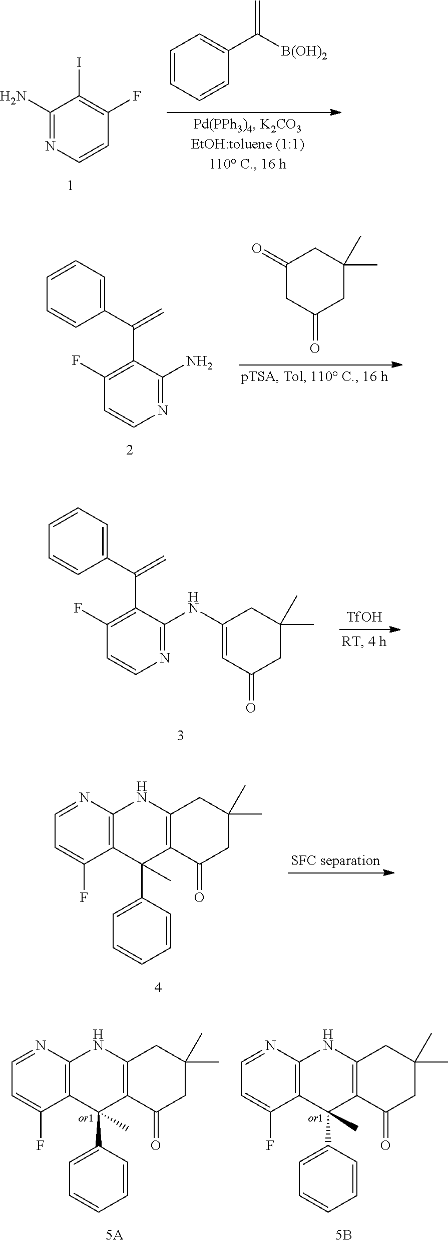

### Step 1: 4-fluoro-3-(1-phenylvinyl)pyridin-2-amine. (2)

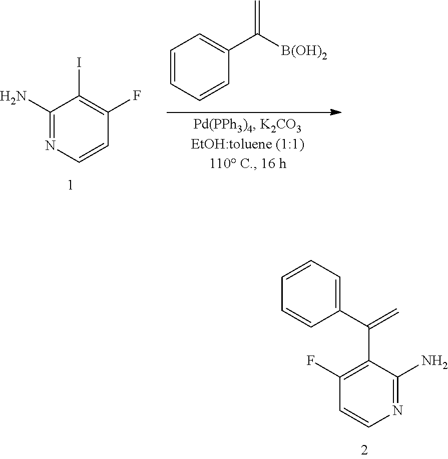

To a stirred solution of 4-fluoro-3-iodo-pyridin-2-amine (150 mg, 0.630 mmol, 1.00 eq) was taken in Toluene (4 mL) and EtOH (4 mL), then 1-phenylvinylboronic acid (84 mg, 0.567 mmol, 0.900 eq), K₂CO₃ (174 mg, 1.26 mmol, 2.00 eq) and Pd(PPh₃)₄ (73 mg, 0.0630 mmol, 0.100 eq) was added to it and the reaction mixture was degassed for 10 minutes and then placed the reaction mixture at 110° C. for 16 h. Progress of the reaction was monitored by TLC and LCMS. Desired compound mass was observed in LCMS. After 2 hours TLC was checked a new polar spot was formed. The mixture was diluted with water and extracted with Ethyl acetate two times. Combined organic layer were dried over Na₂SO₄ to get crude compound. Taken this crude compound and purified using combiflash EtOAc/Hex as an eluent. Compound was eluted at 30% EtOAc/Hex.

Solvent was concentrated under rotavapor to afford 100 mg pure compound.

LCMS: (ES⁺) m/z (M+H)⁺=215.0; tR=7.27 min.

### Step 2: 3-[[4-fluoro-3-(1-phenylvinyl)-2-pyridyl]amino]-5,5-dimethyl-cyclohex-2-en-1-one. (3)

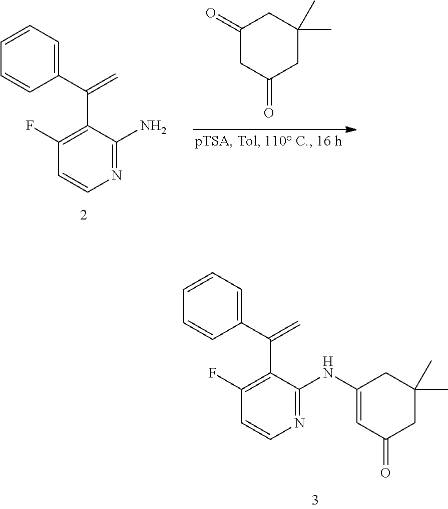

To a stirred solution of 4-fluoro-3-(1-phenylvinyl)pyridin-2-amine (120 mg, 0.560 mmol, 1.00 eq) was taken in Toluene (10 mL), then 5,5-dimethylcyclohexane-1,3-dione (71 mg, 0.504 mmol, 0.900 eq), PTSA (43 mg, 0.224 mmol, 0.400 eq) and Molecular sieve (100 mg, ?, ?) was added to it and placed the reaction mixture at 110° C. for 16 h. Desired compound mass was observed in LCMS. Taken the reaction mass and diluted with water and extracted with Ethyl acetate two times. Combined organic layer were dried over Na₂SO₄ to get crude compound. Taken this crude compound and purified using combiflash Ethyl acetate/hex as an eluent. Compound was eluted at 30% EA/HEX. Solvent was concentrated under rotavapor to afford 3-[[4-fluoro-3-(1-phenylvinyl)-2-pyridyl]amino]-5,5-dimethyl-cyclohex-2-en-1-one (600 mg, 31.84%).

LCMS: tR=8.06 min., (ES⁺) m/z (M+H)⁺=337.0.

### Step 3: (5S)-4-fluoro-5,8,8-trimethyl-5-phenyl-9,10-dihydro-7H-benzo[b][1,8]naphthyridin-6-one. (5A)

### (5R)-4-fluoro-5,8,8-trimethyl-5-phenyl-9,10-dihydro-7H-benzo[b][1,8]naphthyridin-6-one. (5B)

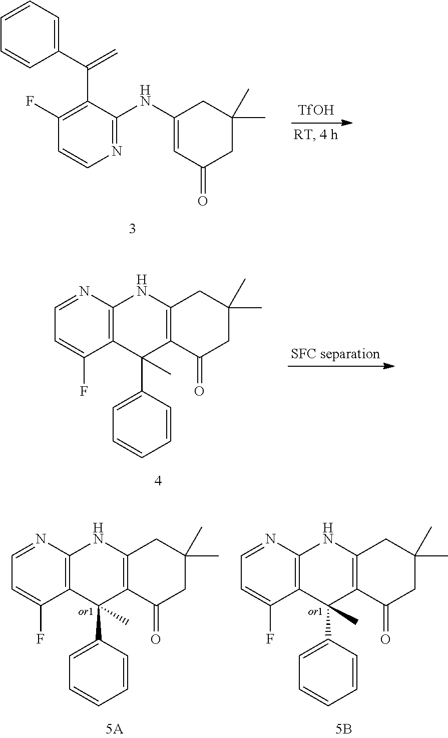

To a stirred solution of 3-[[4-fluoro-3-(1-phenylvinyl)-2-pyridyl]amino]-5,5-dimethyl-PGP-146 C cyclohex-2-en-1-one (180 mg, 0.535 mmol, 1.00 eq) were added Triflic acid (0.94 mL, 10.7 mmol, 20.0 eq) at 0° C. and resulting mixture was allowed to stir at 25° C. for 16 h. After 16 h reaction was monitored by TLC. Mixture was poured into ice cold water and neutralized by NaHCO₃ (pH-7-8), after that aqueous layer was extracted with 10% MeOH in DCM to give crude compound. Crude residue was purified by column chromatography. The desired product was eluted at 30-40% EtOAc in Hexane to give pure compound which was submitted for SFC separation.

The two enantiomers were separated by SFC (Column: CHIRALPAK AS-H (21 mm×250 mm), 5μ; Flow: 60 g/min; Mobile Phase: 75% CO₂+25% (MEOH); ABPR: 110 bar; Temp: 35° C.; UV: 240 nm; diluent: Methanol) to give:

Peak 1: (5S)-4-fluoro-5,8,8-trimethyl-5-phenyl-9,10-dihydro-7H-benzo[b][1,8]naphthyridin-6-one. (5A) (5.4 mg, 0.0154 mmol, 96.33% purity, 3% yield).

LCMS: tR: 2.62 min., (ES⁺) m/z (M+H)⁺=337.28.

HPLC: 96.33%

¹H NMR (400 MHz, DMSO-d6) δ=0.94 (s, 6H) 1.856 (s, 1H) 2.004 (s, 4H) 2.499 (s, 2H), 6.442 (m, 1H), 6.824 (s, 1H) 7.105 (m, 1H) 7.250 (d, 2H) 7.418 (d, 2H) 7.942 (m, 1H).

¹⁹F-NMR=−100.517 ppm.

Peak 2: (5R)-4-fluoro-5,8,8-trimethyl-5-phenyl-9,10-dihydro-7H-benzo[b][1,8]naphthyridin-6-one. (5B) (4.9 mg, 0.0143 mmol, 98.33% purity, 3% yield).

LCMS: (ES⁺) m/z (M+H)⁺=337.28; tR=2.62 min

HPLC=98.33%

¹H NMR (400 MHz, DMSO-d6) δ=0.94 (s, 6H) 1.856 (s, 1H) 2.004 (s, 4H) 2.499 (s, 2H) 6.442 (m, 1H) 6.824 (s, 1H) 7.105 (m, 1H) 7.250 (d, 2H) 7.418 (d, 2H) 7.942 (m, 1H).

¹⁹F-NMR: −100.504 ppm.

### Example 1.20. (5S)-5,8,8-trimethyl-5-phenyl-4-(trifluoromethyl)-9,10-dihydro-7H-benzo[b][1,8]naphthyridin-6-one. (5A)

### (5R)-5,8,8-trimethyl-5-phenyl-4-(trifluoromethyl)-9,10-dihydro-7H-benzo[b][1,8]naphthyridin-6-one. (5B)

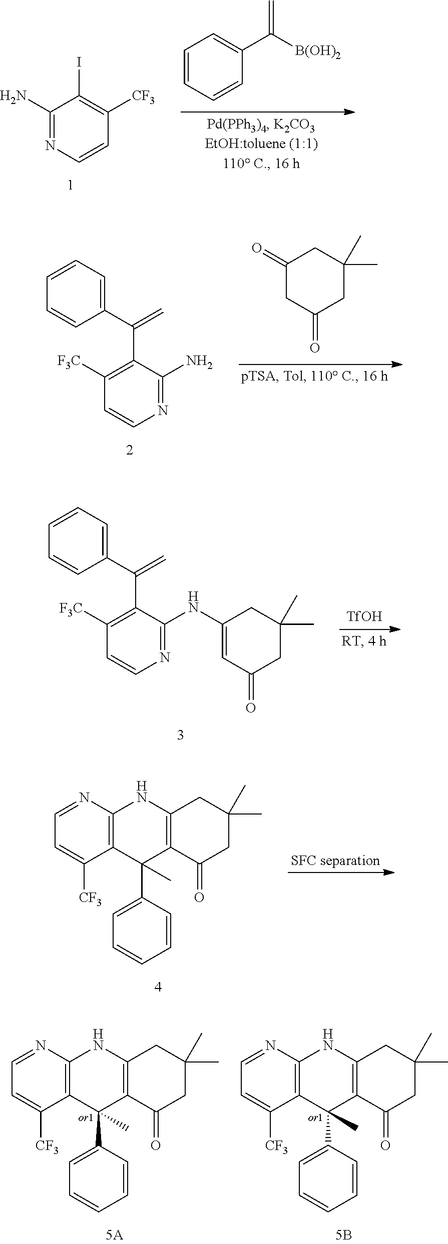

### Step 1: 3-(1-phenylvinyl)-4-(trifluoromethyl)pyridin-2-amine. (2)

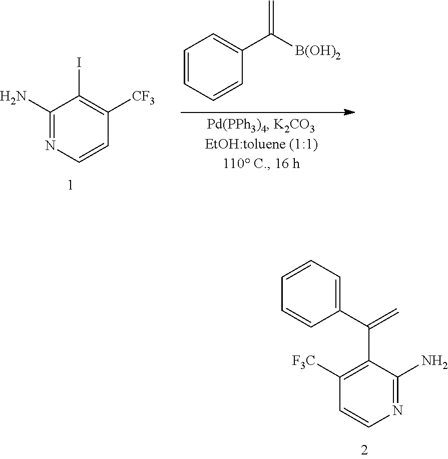 [SMILES: Nc1nccc(C(F)(F)F)c1I ; C=C(B(O)O)c1ccccc1 ; C=C(c1ccccc1)c1c(C(F)(F)F)ccnc1N]

To a stirred solution of 3-iodo-4-(trifluoromethyl)pyridin-2-amine (360 mg, 1.25 mmol, 1.00 eq) in toluene (4 mL) and ethanol (4 mL) were added 1-phenylvinylboronic acid (166 mg, 1.12 mmol, 0.900 eq) and K₂CO₃ (345 mg, 2.50 mmol, 2.00 eq) degassed with Ar for 10 min after that added tetrakis(triphenylphosphine)palladium (144 mg, 0.125 mmol, 0.1000 eq) again degassed for 10 min, resulting mixture was heated at 100° C. for 16 h. After 16 h, the reaction was monitored by TLC and LCMS. 50% Ethyl acetate in hexane shows formation of desired product. Mixture was filtered through cartridge filtrate diluted with water. The aqueous layer was extracted with ethyl acetate. The combined organic layer was dried over Na₂SO₄ and concentrated under reduced pressure to get crude product. Crude residue was purified by combi flash (12 g) chromatography desired product was eluted at 20-30% ethyl acetate in hexane to afford 3-(1-phenylvinyl)-4-(trifluoromethyl)pyridin-2-amine, (120 mg, 0.454 mmol, 100% purity, 36% yield).

LCMS: tR=1.80 min., (ES⁺) m/z (M+H)⁺=265.16.

### Step 2: 5,5-dimethyl-3-[[3-(1-phenylvinyl)-4-(trifluoromethyl)-2-pyridyl]amino]cyclohex-2-en-1-one. (3)

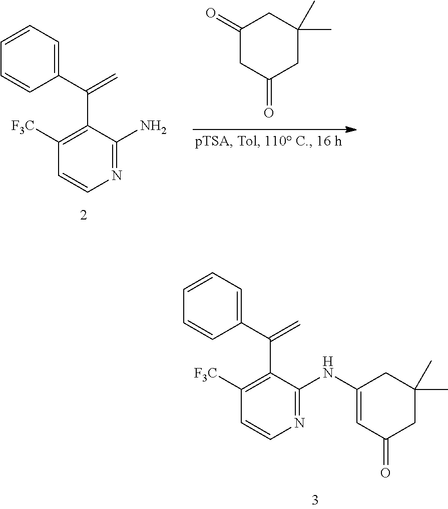

To a stirred solution of 3-(1-phenylvinyl)-4-(trifluoromethyl)pyridin-2-amine (120 mg, 0.454 mmol, 1.0) eq) in toluene (5 mL) were added 5,5-dimethylcyclohexane-1,3-dione (64 mg, 0.454 mmol, 1.00 eq) and p-Toluenesulfonic acid (PTSA) (35 mg, 0.182 mmol, 0.400 eq) resulting mixture was allowed to reflux all 10° C. for 48 h. After 48 h mixture was cooled to RT monitored by TLC and LCMS. 70% EA in hexane shows formation of desired product. Mixture was diluted with ethyl acetate and quenched with saturated NaHCO₃ solution. The aqueous layer was extracted with ethyl acetate. The combined organic layer was washed with brine solution and dried over Na₂SO₄ and concentrated under reduced pressure to get crude compound. Crude residue was purified by combi flash (40 g) chromatography desired product eluted at 60-70% ethyl acetate in hexane to afford white solid 5,5-dimethyl-3-[[3-(1-phenylvinyl)-4-(trifluoromethyl)-2-pyridyl]amino]cyclohex-2-en-1-one, (35 mg, 0.0745 mmol, 16% yield).

LCMS: tR=1.972 min., (ES⁺) m/z (M+H)⁺=387.10.

### Step 3: (5S)-5,8,8-trimethyl-5-phenyl-4-(trifluoromethyl)-9,10-dihydro-7H-benzo[b][1,8]naphthyridin-6-one. (5A)

### (5R)-5,8,8-trimethyl-5-phenyl-4-(trifluoromethyl)-9,10-dihydro-7H-benzo[b][1,8]naphthyridin-6-one. (5B)

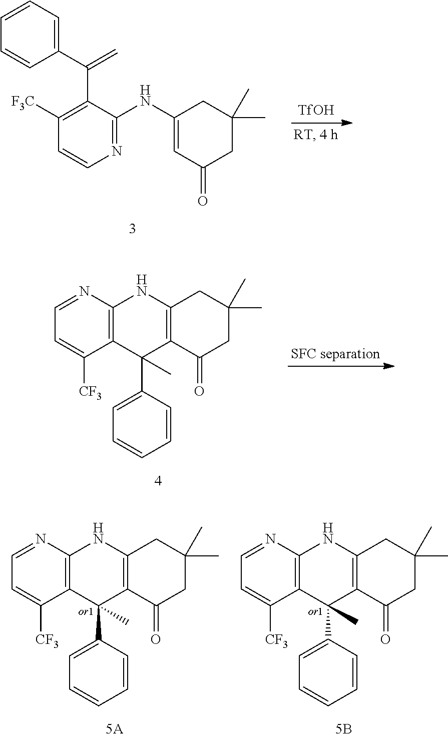

To a stirred solution of 5,5-dimethyl-3-[[3-(1-phenylvinyl)-4-(trifluoromethyl)-2-pyridyl]amino]cyclohex-2-en-1-one (80 mg, 0.207 mmol, 1.00 eq) were added triflic acid (2 mL, 22.7 mmol, 109 eq) at 0° C. and resulting mixture was allowed to stir at 25° C. for 16 h. After 16 h reaction was monitored by TLC. 50% EA in in Hexane shows formation of desired product. Mixture was poured into ice cold water and neutralized by NaHCO₃ (pH-7-8), after that aqueous layer was extracted with 10% MeOH in DCM to give crude compound. Crude residue was purified by column chromatography (4 g) desired product was eluted at 30-40% EA in hexane to give pure compound which was submitted for SFC separation.

The two enantiomers were separated by chiral preparatory SFC (Column: CHIRALPAK AS-H (21 mm×250 mm), 5μ Flow: 60 g/min Mobile Phase: 75% CO₂+25% (MEOH) ABPR: 110 bar; Temp: 35° C.; UV: 240 nm; diluent: Methanol) to give:

Peak 1: (5S)-5,8,8-trimethyl-5-phenyl-4-(trifluoromethyl)-9,10-dihydro-7H-benzo[b][1,8]naphthyridin-6-one. (5A) (17 mg, 0.0446 mmol, 98.9% purity, 22% yield).

LCMS: tR=3.074 min., (ES⁺) m/z (M+H)⁺=387.36.

HPLC: 98.90%

¹H NMR (400 MHz, DMSO-d6) δ=0.862 (s, 3H) 0.962 (s, 3H) 1.919 (d, 1H) 2.025 (d, 1H) 2.327 (s, 3H) 2.503 (d, 2H) 7.015 (d, 1H) 7.155 (m, 3H) 7.280 (d, 2H) 8.302 (d, 1H) 10.082 (s, 1H).

¹⁹F-NMR=−54.670 ppm.

Peak 2: (5R)-5,8,8-trimethyl-5-phenyl-4-(trifluoromethyl)-9,10-dihydro-7H-benzo[b][1,8]naphthyridin-6-one. (5B) (18 mg, 0.0469 mmol, 99.34% purity, 23% yield).

LCMS: tR=3.074 min., (ES⁺) m/z (M+H)⁺=387.36.

HPLC: 99.34%

SFC HPLC: 100%

¹H NMR (400 MHz, DMSO-d6) δ=0.804 (s, 3H) 0.962 (s, 3H) 1.958 (d, 1H) 2.072 (d, 1H) 2.327 (s, 3H) 2.418 (d, 2H) 7.015 (d, 1H) 7.155 (m, 3H) 7.280 (d, 2H) 8.302 (d, 1H) 10.083 (s, 1H).

¹⁹F-NMR=−54.670 ppm.

### Example 1.21. (S)-5,8,8-trimethyl-5-phenyl-4-(2,2,2-trifluoroethyl)-7,8,9,10-tetrahydrobenzo[b][1,8]naphthyridin-6(5H)-one (8A)

### (R)-5,8,8-trimethyl-5-phenyl-4-(2,2,2-trifluoroethyl)-7,8,9,10-tetrahydrobenzo[b][1,8] naphthyridin-6(5H)-one (8B)

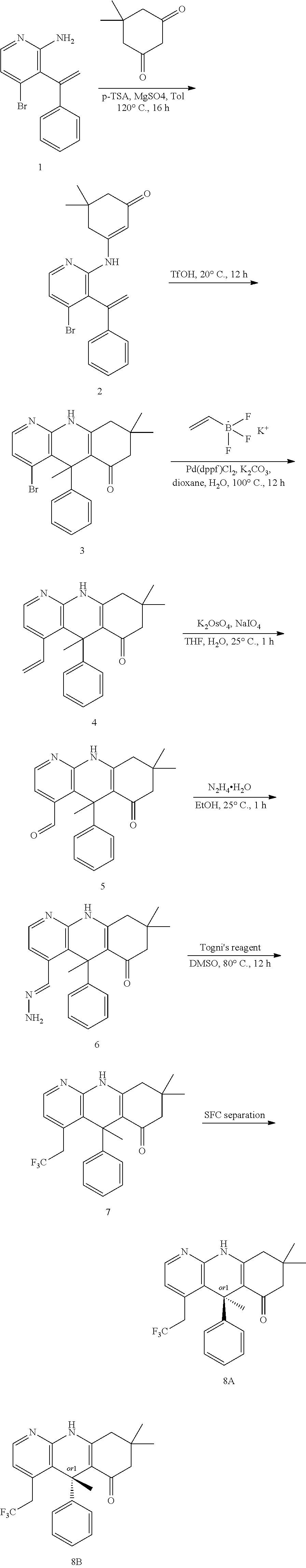

### Step 1: 3-((4-bromo-3-(1-phenylvinyl)pyridin-2-yl)amino)-5,5-dimethylcyclohex-2-en-1-one (2)

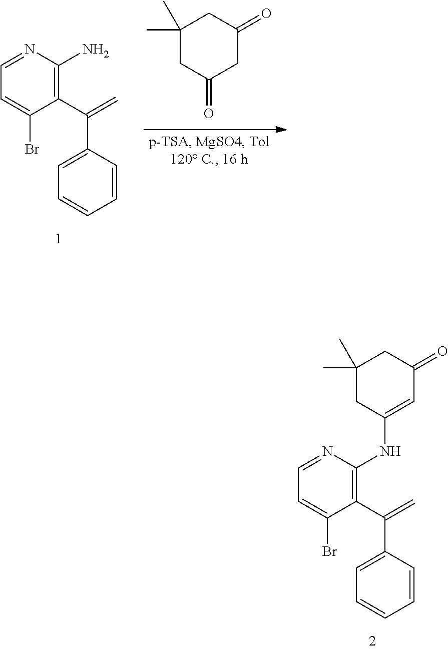

To a solution of compound 1 (1 g, 3.63 mmol, 1 eq) and 5,5-dimethylcyclohexane-1,3-dione (560.42 mg, 4.00 mmol, 1.1 eq) in toluene (10 mL) was added p-TSA (62.59 mg, 363.45 μmol, 0.1 eq) under N₂. The mixture was stirred at 120° C. for 16 h. On completion, the mixture was diluted with H₂O 20 mL and extracted with EtOAc 40 mL (20 mL×2). The combined organic layers were washed with brine 20 mL (10 mL×2), dried over Na₂SO₄, filtered, and concentrated under reduced pressure to give a residue. The residue was purified by flash silica gel chromatography (ISCO®; 20 g SepaFlash® Silica Flash Column, Eluent of 0˜20% Ethyl acetate Petroleum ether gradient @ 80 mL/min) to give compound 2 (770 mg, 53% yield) as a yellow solid.

### Step 2: 4-bromo-5,8,8-trimethyl-5-phenyl-5,8,9,10-tetrahydrobenzo[b][1,8]naphthyridin-6(7H)-one (2)

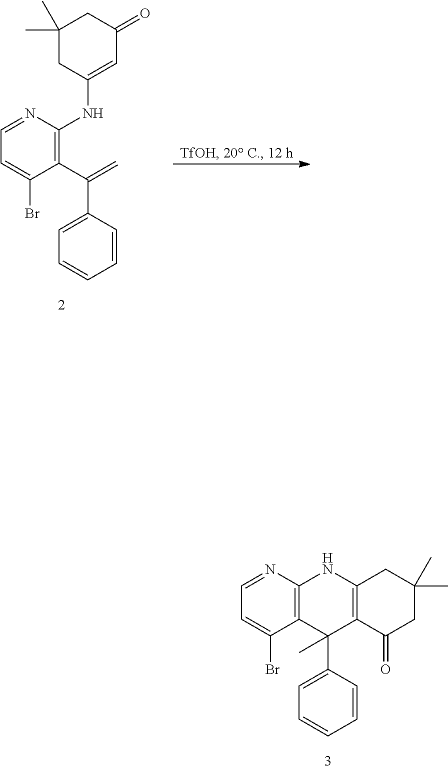 [SMILES: C=C(c1ccccc1)c1c(Br)ccnc1NC1=CC(=O)CC(C)(C)C1 ; CC1(C)CC(=O)C2=C(C1)Nc1nccc(Br)c1C2(C)c1ccccc1]

To a solution of compound 2 (770 mg, 1.94 mmol, 1 eq) in TfOH (10 mL) was stirred at 20° C. for 12 h. On completion, the mixture was diluted with H₂O 20 mL and adjusted pH=8 then extracted with EtOAc 60 mL (30 mL×2). The combined organic layers were washed with H₂O 10 mL and brine, dried over Na₂SO₄, filtered, and concentrated under reduced pressure to give a residue. The residue was purified by flash silica gel chromatography (ISCO®; 20 g SepaFlash® Silica Flash Column, Eluent of 0˜20% Ethyl acetate Petroleum ether gradient @ 80 mL/min) to give compound 2 (190 mg, 25% yield) as a yellow solid.

### Step 3: 5,8,8-trimethyl-5-phenyl-4-vinyl-7,8,9,10-tetrahydrobenzo[b][1,8]naphthyridin-6(5H)-one (4)

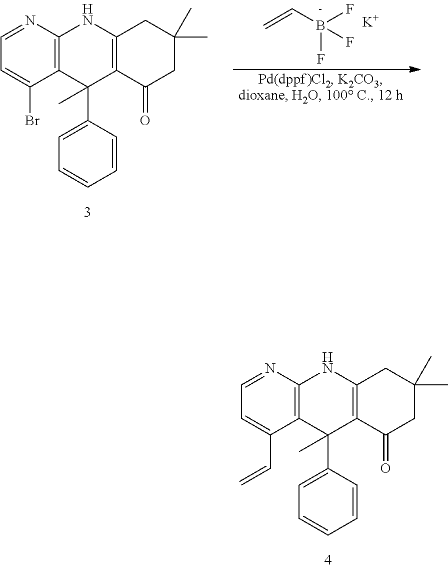

A mixture of compound 3 (810 mg, 2.04 mmol, 1 eq), potassium trifluoro(vinyl)borate (819.26 mg, 6.12 mmol, 3 eq) in dioxane (12 mL) and H₂O (4 mL) was added to Pd(dppf)Cl₂ (149.17 mg, 203.87 μmol, 0.1 eq) and K₂CO₃ (563.53 mg, 4.08 mmol, 2 eq) was degassed and purged with N₂ for 3 times, and then the mixture was stirred at 100° C. for 12 h under N₂ atmosphere. On completion, the mixture was added H₂O (20 mL) and it was extracted with ethyl acetate (10 mL×3). The combined organic phase was washed with brine (30 mL×3), dried over anhydrous sodium sulfate, filtered, and concentrated under reduced pressure. The residue was purified by flash silica gel chromatography (ISCO®; 40 g SepaFlash® Silica Flash Column, Eluent of 0˜26% Ethyl acetate/Petroleum ether gradient @ 45 mL/min) to give compound 4 (536 mg, 75% yield) as a yellow solid.

LCMS: tR=0.440 min., (ES⁺) m/z (M+H)⁺=345.2.

¹H NMR (400 MHz, DMSO-d₆) δ=9.80 (s, 1H), 8.07 (d, J=5.2 Hz, 1H), 7.41 (d, J=7.6 Hz, 2H), 7.26 (t, J=7.6 Hz, 2H), 7.18-7.04 (m, 1H), 6.94 (d, J=5.2 Hz, 1H), 6.37-6.27 (m, 1H), 5.58 (d, J=17.2 Hz, 1H), 5.13 (d, J=12.0 Hz, 1H), 2.47 (d, J=6.8 Hz, 2H), 2.06 (s, 3H), 2.01-1.82 (m, 2H), 1.04 (s, 3H), 0.94 (s, 3H).

### Step 4: 5,8,8-trimethyl-6-oxo-5-phenyl-5,6,7,8,9,10-hexahydrobenzo[b][1,8]naphthyridine-4-carbaldehyde (5)

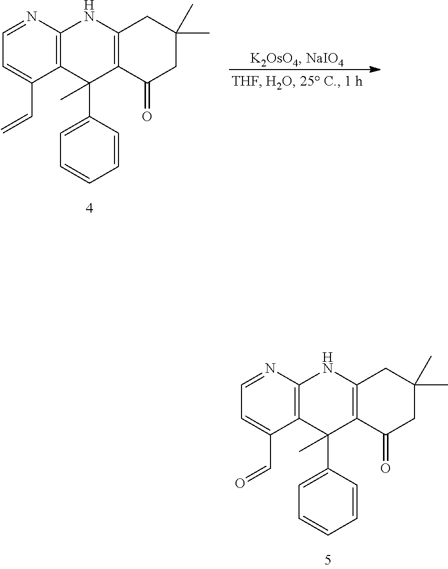

To a solution of compound 3 (370 mg, 1.07 mmol, 1 eq) in THF (2 mL) and H₂O (0.4 mL) was added K₂OsO₄·2H₂O (39.58 mg, 107 μmol, 0.1 eq) and sodium periodate (689 mg, 3.22 mmol, 178 μL, 3 eq). The mixture was stirred at 25° C. for 1 hr. On completion, the mixture was added H₂O (20 mL) and it was extracted with ethyl acetate (10 mL×3). The combined organic phase was washed with brine (30 mL×3), dried over anhydrous sodium sulfate, filtered, and concentrated under reduced pressure. The residue was purified by column chromatography by prep-TLC (SiO₂, Ethyl acetate/Petroleum ether gradient=1:1) to give compound 5 (254 mg, 61% yield) as a yellow solid.

LCMS: tR=0.429 min., (ES⁺) m/z (M+H)⁺=347.3.

### Step 5: (E)-4-(hydrazonomethyl)-5,8,8-trimethyl-5-phenyl-7,8,9,10-tetrahydrobenzo[b][1,8] naphthyridin-6(5H)-one (6)

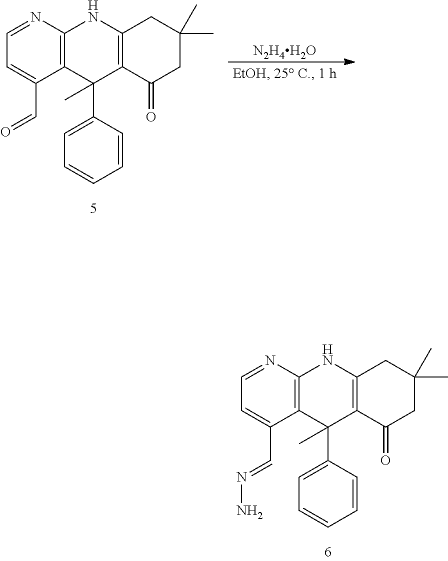 [SMILES: CC1(C)CC(=O)C2=C(C1)Nc1nccc(C=O)c1C2(C)c1ccccc1 ; "N2H4·H2O" ; "EtOH, 25° C., 1 h" ; CC1(C)CC(=O)C2=C(C1)Nc1nccc(/C=N/N)c1C2(C)c1ccccc1]

To a solution of compound 5 (210 mg, 606.20 μmol, 1 eq) in EtOH (2 mL) was slowly added hydrazine hydrate (71.40 mg, 1.21 mmol, 69.32 uL, 85% purity, 2 eq). The mixture was stirred at 25° C. for 1 hr. On completion, the reaction mixture was concentrated under vacuum to give crude product (220 mg) and used for the next step directly.

### Step 6: (S)-5,8,8-trimethyl-5-phenyl-4-(2,2,2-trifluoroethyl)-7,8,9,10-tetrahydrobenzo[b][1,8] naphthyridin-6(5H)-one (8A)

### (R)-5,8,8-trimethyl-5-phenyl-4-(2,2,2-trifluoroethyl)-7,8,9,10-tetrahydrobenzo[b][1,8] naphthyridin-6(5H)-one (8B)

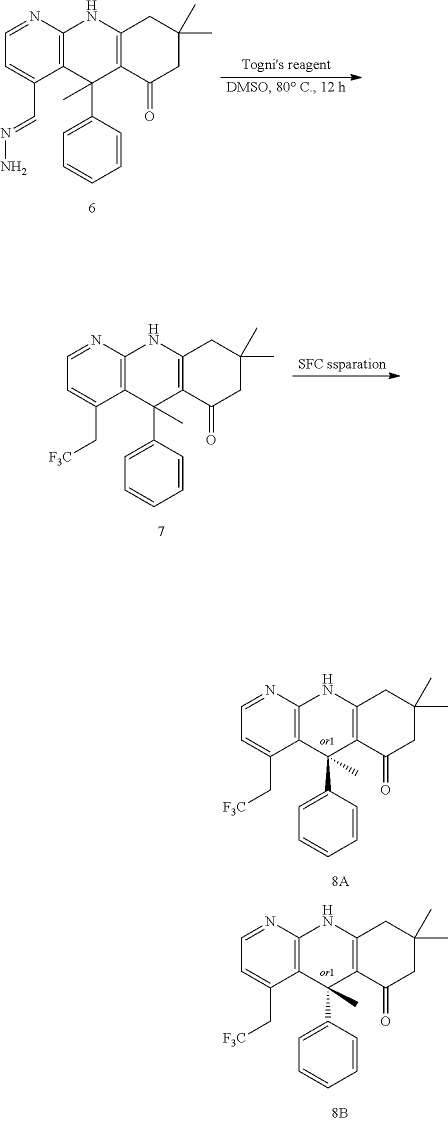

To a solution of compound 4 (220 mg, crude) in DMSO (2 mL) was slowly added 1-(trifluoromethyl)-1,3,2-benziodoxol-3-one (207 mg, 657 μmol, 1.1 eq). The mixture was stirred at 80° C. for 12 h under N₂ atmosphere. On completion, the mixture was added H₂O (20 mL) and it was extracted with ethyl acetate (10 mL×3). The combined organic phase was washed with brine (30 mL), dried over anhydrous sodium sulfate, filtered, and concentrated under reduced pressure.

The residue was purified by prep-HPLC (0.1% FA, 5˜75% ACN/H₂O) to give compound (47 mg, 19.2% yield) as a yellow solid.

LCMS: tR=0.437 min., (ES⁺) m/z (M+H)⁺=401.6.

¹H NMR (400 MHz, DMSO-d₆) δ=9.84 (s, 1H), 8.06 (d, J=5.2 Hz, 1H), 7.36 (br d, J=7.6 Hz, 2H), 7.21 (t, J=7.6 Hz, 2H), 7.11-7.03 (m, 1H), 6.79 (d, J=4.0 Hz, 1H), 3.37 (br s, 1H), 3.18-3.12 (m, 1H), 2.42 (d, J=2.0 Hz, 2H), 1.98 (s, 3H), 1.95-1.80 (m, 2H), 0.98 (s, 3H), 0.89 (s, 3H).

The compound 7 was separated by SFC (AS-3-MeOH(DEA)-5-40-3ML-35T.l cm; “Column: Chiralpak AS-3 50×4.6 mm I.D., 3 um; Mobile phase: Phase A for CO₂, and Phase B for MeOH (0.05% DEA); Gradient elution: B in A from 5% to 40% Flow rate: 3 mL/min; Detector: PDA; Column Temp: 35 C; Back Pressure: 100 Bar) to give:

Peak 1: (S)-5,8,8-trimethyl-5-phenyl-4-(2,2,2-trifluoroethyl)-7,8,9,10-tetrahydrobenzo[b][1,8]naphthyridin-6(5H)-one (10.8 mg, 23% yield).

LCMS: tR=0.484 min., (ES⁺) m/z (M+H)⁺=401.2.

HPLC: (purity: 97.334).

¹H NMR (400 MHz, DMSO-do) δ=9.84 (s, 1H), 8.06 (d, J=5.2 Hz, 1H), 7.36 (br d, J=7.6 Hz, 2H), 7.21 (t, J=7.6 Hz, 2H), 7.11-7.03 (m, 1H), 6.79 (d, J=4.0 Hz, 1H), 3.37 (br s, 1H), 3.18-3.12 (m, 1H), 2.42 (d, J=2.0 Hz, 2H), 1.98 (s, 3H), 1.95-1.80 (m, 2H), 0.98 (s, 3H), 0.89 (s, 3H).

Peak 2: (R)-5,8,8-trimethyl-5-phenyl-4-(2,2,2-trifluoroethyl)-7,8,9,10-tetrahydrobenzo[b][1,8]naphthyridin-6(5H)-one 8B (13.6 mg, 29% yield).

LCMS: tR=0.479 min., (ES⁺) m/z (M+H)⁺=401.1.

HPLC: (purity: 97.334).

¹H NMR (400 MHz, DMSO-dt) δ=9.85 (s, 1H), 8.06 (d, J=5.2 Hz, 1H), 7.35 (br d, J=7.2 Hz, 2H), 7.21 (t, J=8.0 Hz, 2H), 7.11-7.03 (m, 1H), 6.79 (br d, J=4.8 Hz, 1H), 3.40 (br s, 1H), 3.24-3.11 (m, 1H), 2.42 (d, J=1.6 Hz, 2H), 1.98 (s, 3H), 1.96-1.76 (m, 2H), 0.98 (s, 3H), 0.89 (s, 3H).

### Example 1.22. (S)-4-fluoro-5,8,8-trimethyl-5-phenyl-3-(trifluoromethyl)-5,8,9,10-tetrahydrobenzo [b][1,8]naphthyridin-6(7H)-one (9A)

### (R)-4-fluoro-5,8,8-trimethyl-5-phenyl-3-(trifluoromethyl)-5,8,9,10-tetrahydrobenzo [b][1,8]naphthyridin-6(7H)-one (9B)

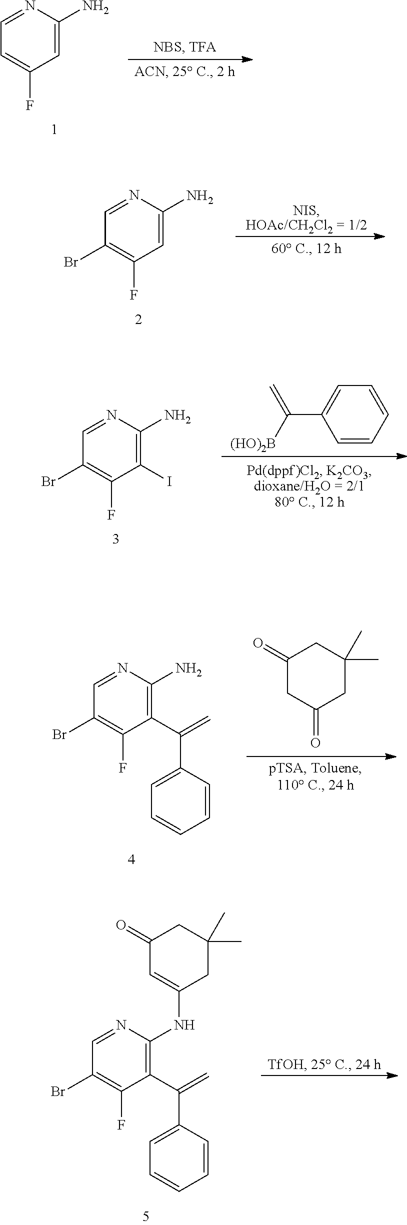

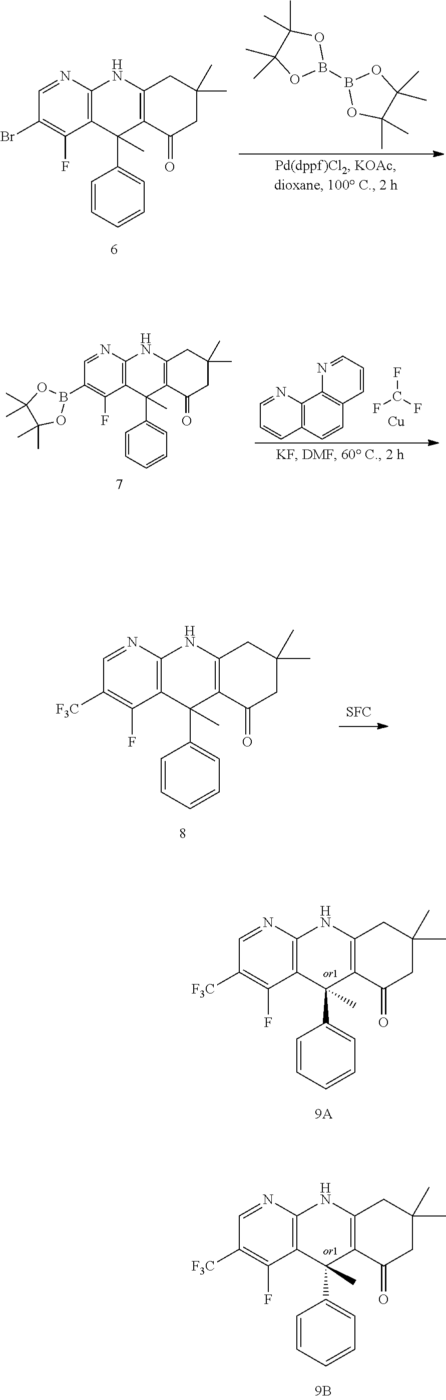

### Step 1: 5-bromo-4-fluoropyridin-2-amine. (2)

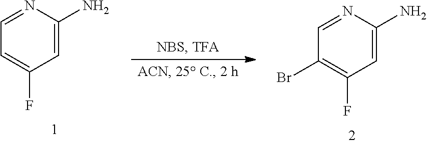

To a solution of 4-fluoropyridin-2-amine (10.0 g, 89.2 mmol, 1.00 eq) and NBS (15.8 g, 89.2 mmol, 1.00 eq) in ACN (100 mL) was added TFA (10.17 g, 89.2 mmol, 1.00 eq) dropwise with the temperature kept at 25° C. to get a red solution. The reaction mixture was stirred at 25° C. for 2 hours. On completion, the reaction mixture was concentrated directly to remove ACN, then it was dissolved with EtOAc (50 mL) and H₂O (50 mL). The aqueous layer was extracted with EtOAc (30 mL×3), the combined organic layers were washed with brine (30 mL×3), dried over Na₂SO₄, filtered, and concentrated under reduced pressure to give a residue. The yellow residue was purified by column chromatography (SiO₂, Petroleum ether/Ethyl acetate=3:1) to give compound 2 (11.0 g, 65% yield) as a brown solid.

¹H NMR (400 MHz, DMSO-d₆) δ=8.04 (d, J=10.4 Hz, 1H), 6.46 (br s, 2H), 6.35 (d, J=11.2 Hz, 1H).

### Step 2: 5-bromo-4-fluoro-3-iodopyridin-2-amine (3)

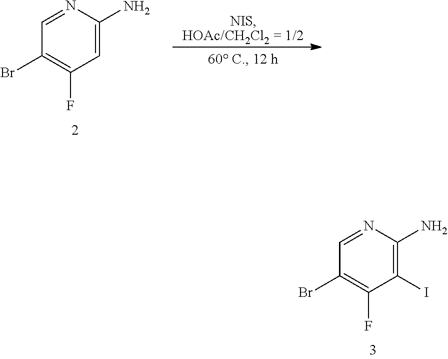

To a solution of compound 2 (5.00 g, 26.2 mmol, 1.00 eq) in HOAc (50 mL) and CH₂Cl₂ (100 mL) was added NIS (5.89 g, 26.2 mmol, 1.00 eq) portion wise with the temperature kept at 25° C. to get a red solution. The reaction mixture was stirred at 60° C. for 12 hours. On completion, the reaction mixture was concentrated directly to remove CH₂Cl₂ and HOAc, then it was dissolved with EtOAc (50 mL) and quenched with saturated Na₂CO₃ (100 mL), and then extracted with EtOAc (30 mL×2). The combined organic layers were washed with brine (30 mL×3), dried over Na₂SO₄, filtered, and concentrated under reduced pressure to give a residue. The yellow residue was purified by column chromatography (SiO₂, Petroleum ether/Ethyl acetate=5:1) to give compound 3 (5.10 g, 61% yield) as a yellow solid.

¹H NMR (400 MHz, DMSO-d₆) δ=8.01 (d, J=9.6 Hz, 1H), 6.62 (br s, 2H).

### Step 3: 5-bromo-4-fluoro-3-(1-phenylvinyl)pyridin-2-amine (4)

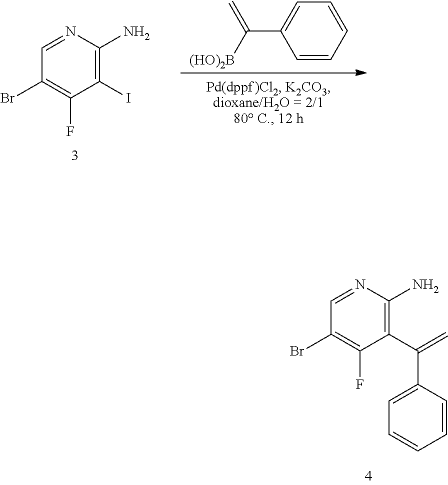

To a solution of 1-phenylvinylboronic acid (2.78 g, 18.8 mmol, 1.20 eq) and compound 3 (5.00 g, 15.7 mmol, 1.00 eq) in dioxane (50 mL) and H₂O (25 mL) was added K₂CO₃ (6.49 g, 47.0 mmol, 3.00 eq) and Pd(dppf)Cl₂ (1.15 g, 1.57 mmol, 0.100 eq) portion wise with the temperature kept at 25° C. under N₂ to get a yellow solution. The reaction mixture was stirred at 80° C. for 12 hours. On completion, the reaction mixture was quenched with NH₄Cl (25 mL), and then extracted with EtOAc (50 mL×3). The combined organic layers were washed with brine (50 mL×3), dried over Na₂SO₄, filtered, and concentrated under reduced pressure to give a residue. The yellow residue was purified by column chromatography (SiO2, Petroleum ether/Ethyl acetate=0:1-5:1) to give compound 4 (3.70 g, 81% yield) as a yellow solid.

¹H NMR (400 MHz, DMSO-d₆) δ=8.13 (d, J=9.6 Hz, 1H), 7.37-7.31 (m, 5H), 6.13 (s, 1H), 6.09 (br s, 2H), 5.43 (s, 1H).

### Step 4: 3-((5-bromo-4-fluoro-3-(1-phenylvinyl)pyridin-2-yl)amino)-5,5-dimethylcyclohex-2-en-1-one (5)

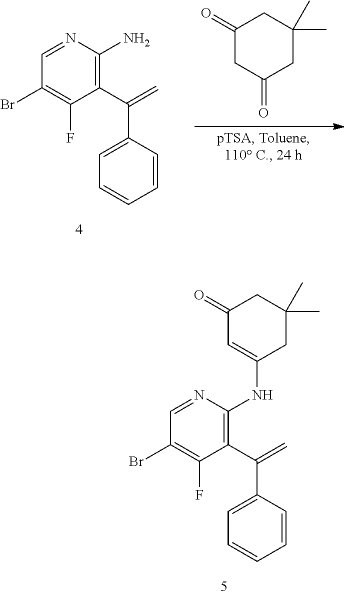

To a solution of 5,5-dimethylcyclohexane-1,3-dione (1.77 g, 12.6 mmol, 1.00 eq) and compound 4 (3.70 g, 12.6 mmol, 1.00 eq) in toluene (37 mL) was added pTSA (0.21 g, 1.26 mmol, 0.100 eq) portion wise with the temperature kept at 25° C. under N₂ to get a yellow solution. The reaction mixture was stirred at 110° C. for 24 hours. On completion, the reaction mixture was quenched with H₂O (10 mL), and then extracted with EtOAc (30 mL×3). The combined organic layers were washed with brine (30 mL×3), dried over Na₂SO₄, filtered, and concentrated under reduced pressure to give a residue. The yellow residue was purified by column chromatography (SiO₂, Petroleum ether/Ethyl acetate=0:1-3:1) to give compound 5 (1.80 g, 4.33 mmol, 34% yield) as a yellow solid.

LCMS: tR=0.455 min., (ES⁺) m/z (M+H)⁺=415.

¹H NMR (400 MHz, DMSO-do) δ=8.62 (d, J=9.2 Hz, 1H), 8.05 (s, 1H), 7.34-7.28 (m, 5H), 6.23 (d, J=12.0 Hz, 2H), 5.59 (s, 1H), 2.21 (s, 2H), 2.18 (s, 2H), 0.85 (s, 6H).

### Step 5: 3-bromo-4-fluoro-5,8,8-trimethyl-5-phenyl-7,8,9,10-tetrahydrobenzo[b][1,8] naphthyridin-6(5H)-one (6)

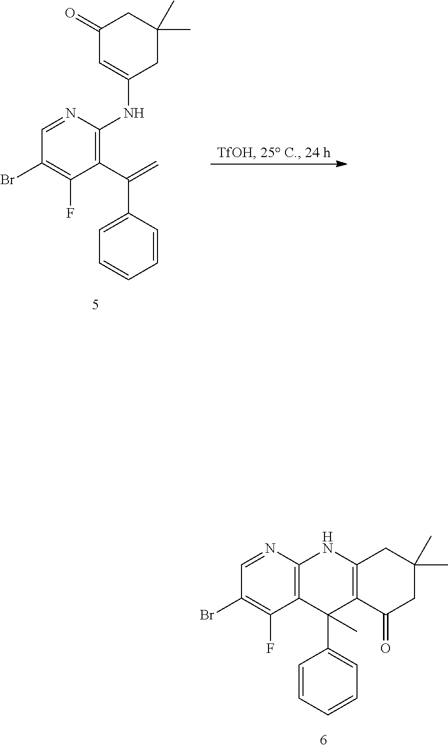 [SMILES: C=C(c1ccccc1)c1c(NC2=CC(=O)CC(C)(C)C2)ncc(Br)c1F ; CC1(C)CC(=O)C2=C(C1)Nc1ncc(Br)c(F)c1C2(C)c1ccccc1]

To a solution of compound 5 (1.80 g, 4.33 mmol, 1.00 eq) was added TfOH (18 mL) portion wise with the temperature kept at 25° C. to get a yellow solution. The reaction mixture was stirred at 25° C. for 24 hours. On completion, the reaction mixture was quenched with NaOH (2.5 M, 15 mL), and then extracted with EtOAc (20 mL×3). The combined organic layers were washed with brine (20 mL×3), dried over Na₂SO₄, filtered, and concentrated under reduced pressure to give a residue. The yellow residue was purified by column chromatography (SiO₂, Petroleum ether/Ethyl acetate=0:1-5:1) to give compound 6 (1.20 g, 67% yield) as a yellow solid.

¹H NMR (400 MHz, DMSO-d₆) δ=10.15 (s, 1H), 8.26 (d, J=8.8 Hz, 1H), 7.34 (d, J=7.6 Hz, 2H), 7.22 (t, J=7.6 Hz, 2H), 7.10-7.04 (m, 1H), 2.46 (s, 2H), 2.02-1.99 (m, 4H), 1.91 (s, 1H), 0.99 (s, 3H), 0.94 (s, 3H).

### Step 6: (4-fluoro-5,8,8-trimethyl-6-oxo-5-phenyl-5,6,7,8,9,10-hexahydrobenzo[b][1,8] naphthyridin-3-yl)boronic acid (7)

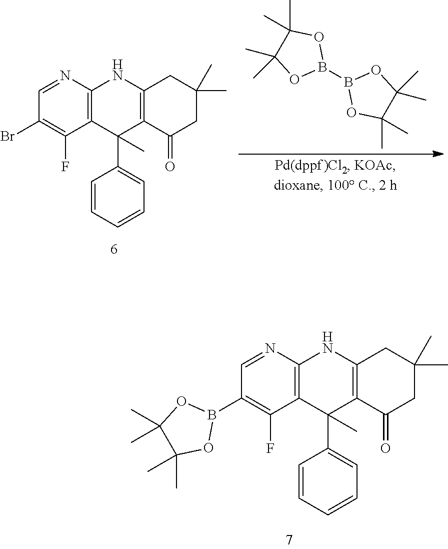

To a solution of compound 6 (500 mg, 1.20 mmol, 1.00 eq) and 4,4,5,5-tetramethyl-2-(4,4,5,5-tetramethyl-1,3,2-dioxaborolan-2-yl)-1,3,2-dioxaborolane (611 mg, 2.41 mmol, 2.00 eq) in dioxane (5 mL) was added KOAc (590 mg, 6.02 mmol, 5.00 eq) and Pd(dppf)Cl₂ (88 mg, 0.120 mmol, 0.100 eq) portion wise with the temperature kept at 25° C. under N₂ to get a yellow solution. The reaction mixture was stirred at 100° C. for 12 hours. On completion, the reaction mixture was quenched with NH₄Cl (5 mL), and then extracted with EtOAc (5 mL×3). The combined organic layers were washed with brine (5 mL×3), dried over Na₂SO₄, filtered, and concentrated under reduced pressure to give a residue. The yellow residue was purified by column chromatography (SiO2, Petroleum ether/Ethyl acetate=0:1-1:1) to give compound 7 (230 mg, 50% yield) as a yellow solid.

### Step 7: (S)-4-fluoro-5,8,8-trimethyl-5-phenyl-3-(trifluoromethyl)-7,8,9,10-tetrahydrobenzo [b][1,8]naphthyridin-6(5H)-one (9A) & (R)-4-fluoro-5,8,8-trimethyl-5-phenyl-3-(trifluoromethyl)-7,8,9,10-tetrahydrobenzo[b][1,8] naphthyridin-6(5H)-one (9B)

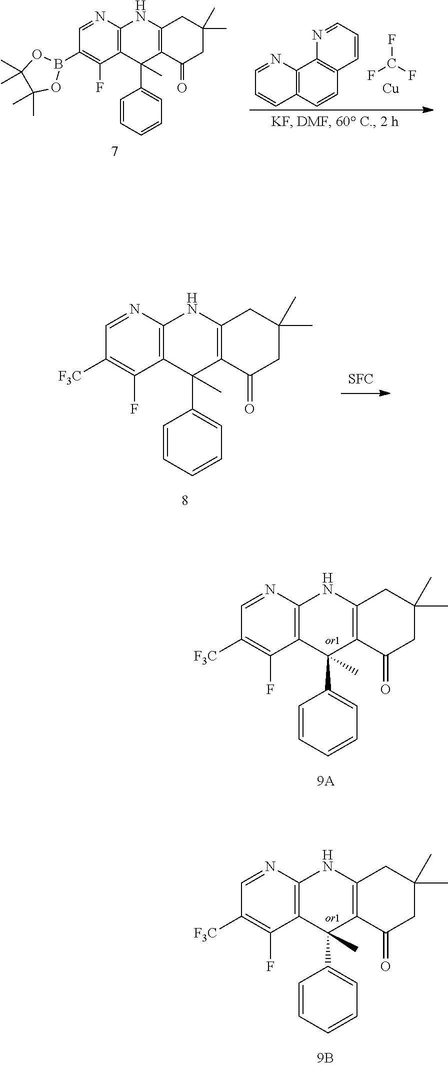

To a solution of compound 7 (230 mg, 0.605 mmol, 1.00 eq) and trifluoromethyl(1,10-phenanthroline)copper (188 mg, 0.605 mmol, 1.00 eq) in DMF (2.5 mL) was added KF (35 mg, 0.605 mmol, 1.00 eq) portion wise with the temperature kept at 25° C. under N₂ to get a black solution. The reaction mixture was stirred at 60° C. for 2 hours. On completion, the reaction mixture was quenched with NH₄Cl (5 mL), and then extracted with EtOAc (5 mL×3). The combined organic layers were washed with brine (5 mL×3), dried over Na₂SO₄, filtered, and concentrated under reduced pressure to give a residue. The residue was purified by re-MPLC (5˜100% ACN/H₂O(FA)) to give compound 8 (40 mg, 16% yield).

SFC separation (Column: (S,S)Whelk-O1 50×46 mm I.D., 3.5 um, 100 Bar mobile phase: [0.05% DEA/MeOH]; B %: 5%-40%, B5.5; 180 min) afforded:

Peak 1: (S)-4-fluoro-5,8,8-trimethyl-5-phenyl-3-(trifluoromethyl)-7,8,9,10-tetrahydrobenzo [b][1,8]naphthyridin-6(5H)-one (9A) (5.6 mg, 0.0136 mmol, 14% yield) as a white solid.

LCMS: tR=0.537 min., (ES⁺) m/z (M+H)⁺=405.1.

¹H NMR (400 MHz, DMSO-d₆) δ=10.47 (br s, 1H), 8.41 (d, J=9.2 Hz, 1H), 7.36 (d, J=7.6 Hz, 2H), 7.23 (t, J=7.6 Hz, 2H), 7.11-7.05 (m, 1H), 2.63-2.55 (m, 2H), 2.06-1.99 (m, 4H), 1.94-1.87 (m, 1H), 0.98 (d, J=19.8 Hz, 6H).

Peak 2: (R)-4-fluoro-5,8,8-trimethyl-5-phenyl-3-(trifluoromethyl)-7,8,9,10-tetrahydrobenzo[b][1,8]naphthyridin-6(5H)-one (9B) (13 mg, 0.0310 mmol, 31% yield) as a white solid.

LCMS: tR=0.537 min., (ES⁺) m/z (M+H)⁺=405.1.

¹H NMR (400 MHz, DMSO-d₆) δ=10.47 (br s, 1H), 8.41 (d, J=9.6 Hz, 1H), 7.40-7.33 (m, 2H), 7.23 (t, J=7.6 Hz, 2H), 7.13-7.04 (m, 1H), 2.77-2.70 (m, 2H), 2.06-1.99 (m, 4H), 1.95-1.88 (m, 1H), 0.98 (d, J=19.6 Hz, 6H).

### Example 1.23. (S)-3-bromo-4-fluoro-5,8,8-trimethyl-5-phenyl-7,8,9,10-tetrahydrobenzo[b][1,8]naphthyridin-6(5H)-one (6A)

### (R)-3-bromo-4-fluoro-5,8,8-trimethyl-5-phenyl-7,8,9,10-tetrahydrobenzo[b][1,8] naphthyridin-6(5H)-one (6B)

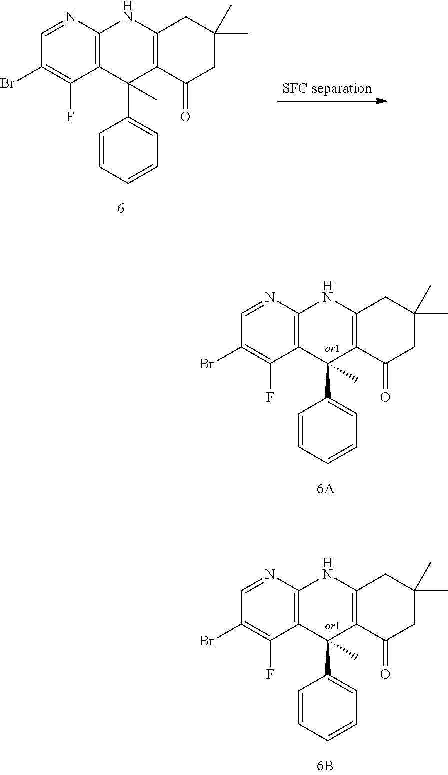 [SMILES: CC1(C)CC(=O)C2=C(C1)Nc1ncc(Br)c(F)c1C2(C)c1ccccc1 ; "SFC separation" ; CC1(C)CC(=O)C2=C(C1)Nc1ncc(Br)c(F)c1[C@]2(C)c1ccccc1 ; CC1(C)CC(=O)C2=C(C1)Nc1ncc(Br)c(F)c1[C@]2(C)c1ccccc1]

120 mg 6 was separated by SFC (Chiralpak AD-3 50×4.6 mm I.D., 3 um; Mobile phase: EtOH (0.05% DEA) in CO₂ from 5% to 40%; Flow rate: 3 mL/min Wavelength: 220 nm) to give:

Peak 1: (S)-3-bromo-4-fluoro-5,8,8-trimethyl-5-phenyl-7,8,9,10-tetrahydrobenzo[b][1,8] naphthyridin-6(5H)-one. (6A) (22 mg, 18% yield).

LCMS: tR=0.450 min., (ES⁺) m/z (M+H)⁺=416.0.

¹H NMR (400 MHz, DMSO-d₆) δ=10.16 (s, 1H), 8.26 (d, J=8.4 Hz, 1H), 7.34 (d, J=7.6 Hz, 2H), 7.22 (t, J=7.6 Hz, 2H), 7.09-7.03 (m, 1H), 2.46 (s, 2H), 2.04-1.99 (m, 4H), 1.92-1.86 (m, 1H), 1.00-0.93 (m, 6H).

Peak 2: (R)-3-bromo-4-fluoro-5,8,8-trimethyl-5-phenyl-7,8,9,10-tetrahydrobenzo[b][1,8] naphthyridin-6(5H)-one. (6B) (28 mg, 23% yield).

LCMS: tR=0.452 min., (ES⁺) m/z (M+H)⁺=415.9.

¹H NMR (400 MHz, DMSO-d₆) δ=10.16 (s, 1H), 8.26 (d, J=8.8 Hz, 1H), 7.34 (d, J=7.6 Hz, 2H), 7.22 (t, J=7.6 Hz, 2H), 7.10-7.03 (m, 1H), 2.45 (s, 2H), 2.04-1.98 (m, 4H), 1.92-1.85 (m, 1H), 1.02-0.91 (m, 6H).

### Example 1.24. (S)-3-fluoro-5,8,8-trimethyl-5-phenyl-4-(trifluoromethyl)-7,8,9,10 tetrahydrobenzo[b][1,8]naphthyridin-6(5H)-one (2A)

### (R)-3-fluoro-5,8,8-trimethyl-5-phenyl-4-(trifluoromethyl)-7,8,9,10-tetrahydrobenzo[b][1,8]naphthyridin-6(5H)-one (2B)

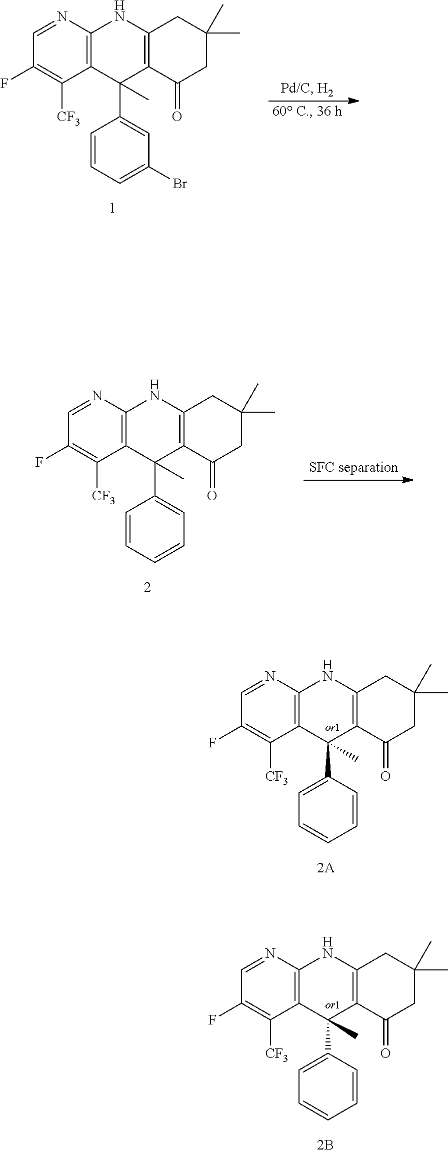

To a solution of compound 1 (130 mg, 0.27 mmol, 1.0 eq) in MeOH (2 mL) was added 10% Pd/C (130 mg, 1.23 mmol, 4.56 eq) portion wise to the reaction mixture. The reaction mixture was stirred at 60° C. for 36 h. On completion, the mixture was filtered, and the filter cake was dried under vacuum. The residue was purified by prep-HPLC to give compound 2 (70 mg, 55% yield) as a white solid.

70 mg 2 was separated by SFC ((Method-B\AD-3-IPA(DEA)-5-40-3 mL-35T.lcm) Column: Chiralpak AS-3 50×4.6 mm I.D., 3 um, Mobile phase: Phase A for CO₂, and Phase B for IPA (0.05% DEA); Gradient elution: B in A from 5% to 40%, Flow rate: 3 mL/min; Detector: PDA; Column Temp: 35 C; Back Pressure: 100 Bar) to give:

Peak 1: (S)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (2A). (8.7 mg, 12% yield).

LCMS: tR=0.512 min., (ES⁺) m/z (M+H)⁺=405.0.

¹H NMR (400 MHz, DMSO-d₆) δ=10.14 (br s, 1H), 8.45 (s, 1H), 7.25 (br d, J=6.4 Hz, 2H), 7.15 (br t, J=7.6 Hz, 2H), 7.07-6.97 (m, 1H), 2.44-2.33 (m, 2H), 2.08 (s, 3H), 1.99-1.91 (m, 1H), 1.86-1.76 (m, 1H), 0.95 (s, 3H), 0.80 (s, 3H).

Peak 2: (R)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (2B). (7.5 mg, 11% yield).

LCMS: tR=0.511 min., (ES⁺) m/z (M+H)⁺=405.2.

¹H NMR (400 MHz, DMSO-d) δ=10.14 (s, 1H), 8.45 (s, 1H), 7.25 (br d, J=7.2 Hz, 2H), 7.15 (t, J=7.6 Hz, 2H), 7.08-6.98 (m, 1H), 2.43-2.32 (m, 2H), 2.08 (s, 3H), 1.99-1.92 (m, 1H), 1.87-1.78 (m, 1H), 0.95 (s, 3H), 0.80 (s, 3H).

### Example 1.25. (S)-5,8,8-trimethyl-5-(3-(methylsulfonyl)phenyl)-3-(trifluoromethyl)-5,8,9,10-tetrahydrobenzo[b][1,8]naphthyridin-6(7H)-one (2A)

### (R)-5,8,8-trimethyl-5-(3-(methylsulfonyl)phenyl)-3-(trifluoromethyl)-5,8,9,10-tetrahydrobenzo[b][1,8]naphthyridin-6(7H)-one (2B)

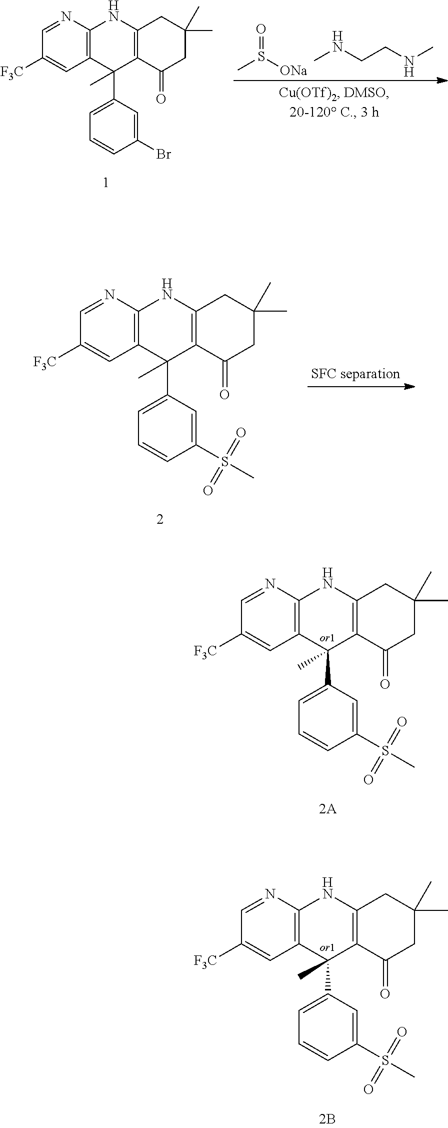

To a solution of sodium methanesulfinate (132 mg, 1.29 mmol, 2.00 eq) in DMSO (3 mL) was added Cu(OTf)₂ (233 mg, 0.65 mmol, 1.00 eq) and N1,N2-dimethylethane-1,2-diamine (119 mg, 1.35 mmol, 2.10 eq) in one portion stirred under N₂ at 20° C. for 0.08 h. Then a solution of compound 1 (300 mg, 0.65 mmol, 1.00 eq) in DMSO (3 mL) was added dropwise to the mixture, the mixture was stirred under N₂ at 120° C. for 3 h. On completion, the reaction was diluted with water (20 mL), extracted with EtOAc (3×20 mL), washed with NH₃·H₂O (30 mL) washed with saturated NaCl (30 mL), dried over Na₂SO₄, filtered, and concentrated under reduced pressure to give compound 2 (150 mg, 50% yield).

Compound 2 (150 mg) was separated by SFC (IG-3-MeOH (DEA)-5-40-3 mL-35T.lcm; “Column: Chiralpak IG-3 50×4.6 mm I.D., 3 um Mobile phase: Phase A for CO₂, and Phase B for MeOH (0.05% DEA); Gradient elution: B in A from 5% to 40% Flow rate: 3 mL/min; Detector: PDA; Column Temp: 35 C; Back Pressure: 100 Bar”) to give:

Peak 1: (S)-5,8,8-trimethyl-5-(3-(methylsulfonyl)phenyl)-3-(trifluoromethyl)-5,8,9,10-tetrahydrobenzo[b][1,8]naphthyridin-6(7H)-one (2A). (31 mg, 20% yield).

LCMS: tR=0.419 min., (ES⁺) m/z (M+H)⁺=465.3.

¹H NMR (400 MHz, DMSO-d6) δ=10.36 (s, 1H), 8.38 (s, 1H), 7.90 (s, 1H), 7.71 (br dd, J=8.0, 13.2 Hz, 2H), 7.59-7.52 (m, 1H), 7.37 (d, J=1.6 Hz, 1H), 3.16 (s, 3H), 2.54 (s, 2H), 2.10-2.05 (m, 1H), 2.01-1.95 (m, 4H), 1.02 (d, J=6.0 Hz, 6H).

Peak 2: (R)-5,8,8-trimethyl-5-(3-(methylsulfonyl)phenyl)-3-(trifluoromethyl)-5,8,9,10-tetrahydrobenzo [b][1,8]naphthyridin-6(7H)-one (2B). (40 mg, 26% yield).

LCMS: tR=0.413 min., (ES⁺) m/z (M+H)⁺=465.5.

¹H NMR (400 MHz, DMSO-d₆) δ=10.36 (br s, 1H), 8.38 (d, J=1.2 Hz, 1H), 7.90 (s, 11H), 7.71 (br dd, J=8.0, 13.2 Hz, 2H), 7.59-7.51 (m, 1H), 7.37 (d, J=2.0 Hz, 1H), 3.16 (s, 3H), 2.54 (s, 2H), 2.10-2.05 (m, 1H), 2.03-1.95 (m, 4H), 1.02 (br d, J=6.0 Hz, 6H).

### Example 1.26. (S)-3-fluoro-5,8,8-trimethyl-5-(3-(methylsulfonyl)phenyl)-5,8,9,10-tetrahydrobenzo[b][1,8]naphthyridin-6(7H)-one (2A)

### (R)-3-fluoro-5,8,8-trimethyl-5-(3-(methylsulfonyl)phenyl)-5,8,9,10-tetrahydrobenzo[b][1,8]naphthyridin-6(7H)-one (2B)

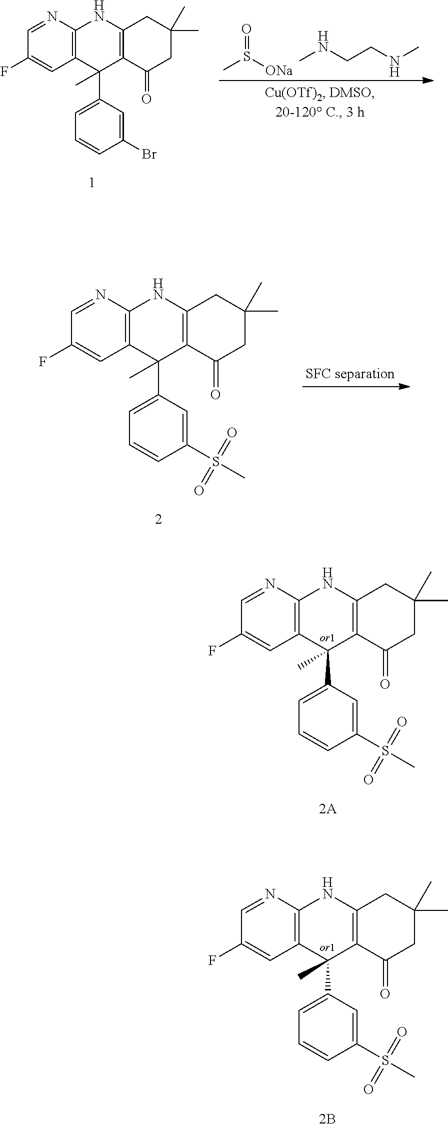

To a solution of N,N′-dimethylethane-1,2-diamine (134 mg, 1.52 mmol, 2.10 eq) and methylsulfinyloxysodium (147 mg, 1.44 mmol, 2.00 eq) in DMSO (4 mL) was added Cu(OTf)₂ (261 mg, 0.721 mmol, 0.998 eq) under N₂ in one portion at 25° C. Then 1 (300 mg, 0.722 mmol, 1.00 eq) was added to the mixture. The mixture was stirred at 120° C. for 3 h. On completion, the reaction mixture was poured into NH₃H₂O (5 mL) and extracted with EtOAc (5 mL×3). The combined organic layer was washed with brine (20 mL), dried over Na₂SO₄, filtered, and concentrated under reduced pressure to give compound 2 (208 mg, 63% yield) as a yellow solid.

LCMS: tR=0.392 min., (ES⁺) m/z (M+H)⁺=415.1.

The product (208 mg) was separated by SFC (Column: Chiralpak AS-3 50×4.6 mm I.D., 3 um, Mobile phase: Phase A for CO₂, and Phase B for EtOH (0.05% DEA); Gradient elution: B in A from 5% to 40%, Flow rate: 3 mL/min) to give:

Peak 1: (S)-3-fluoro-5,8,8-trimethyl-5-(3-(methylsulfonyl)phenyl)-5,8,9,10-tetrahydrobenzo[b][1,8]naphthyridin-6(7H)-one (2A). (22.7 mg, 11% yield).

LCMS: tR=0.394 min., (ES⁺) m/z (M+H)⁺=415.1.

¹H NMR (400 MHz, DMSO-d₆) δ=10.02 (s, 1H), 8.03 (d, J=2.8 Hz, 1H), 7.96 (s, 1H), 7.88 (s, 1H), 7.71-7.67 (m, 2H), 7.56-7.51 (m, 1H), 7.11 (dd, J=2.8, 9.2 Hz, 1H), 3.18 (s, 3H), 2.90 (s, 1H), 2.74 (s, 1H), 2.61-2.57 (m, 2H), 2.08-2.03 (m, 1H), 1.99-1.93 (m, 4H), 1.01 (d, J=7.2 Hz, 6H).

Peak 2: (R)-3-fluoro-5,8,8-trimethyl-5-(3-(methylsulfonyl)phenyl)-5,8,9,10-tetrahydrobenzo[b][1,8]naphthyridin-6(7H)-one (2B). (53.2 mg, 26% yield).

LCMS: tR=0.390 min., (ES⁺) m/z (M+H)⁺=415.1.

¹H NMR (400 MHz, DMSO-d₆) δ=10.02 (s, 1H), 8.02 (d, J=2.8 Hz, 1H), 7.87 (t, J=1.6 Hz, 1H), 7.68 (td, J=1.6, 7.6 Hz, 2H), 7.55-7.50 (m, 1H), 7.10 (dd, J=2.8, 9.2 Hz, 1H), 3.18 (s, 3H), 2.48-2.42 (m, 2H), 2.07-2.02 (m, 1H), 1.98-1.91 (m, 4H), 1.01 (d, J=7.2 Hz, 6H).

### Example 1.27. Synthesis of (S)-4-chloro-5,8,8-trimethyl-5-phenyl-3-(trifluoromethyl)-5,8,9,10-tetrahydrobenzo [b][1,8]naphthyridin-6(7H)-one (7A) & (R)-4-chloro-5,8,8-trimethyl-5-phenyl-3-(trifluoromethyl)-5,8,9,10-tetrahydrobenzo[b][1,8] naphthyridin-6(7H)-one (7B)

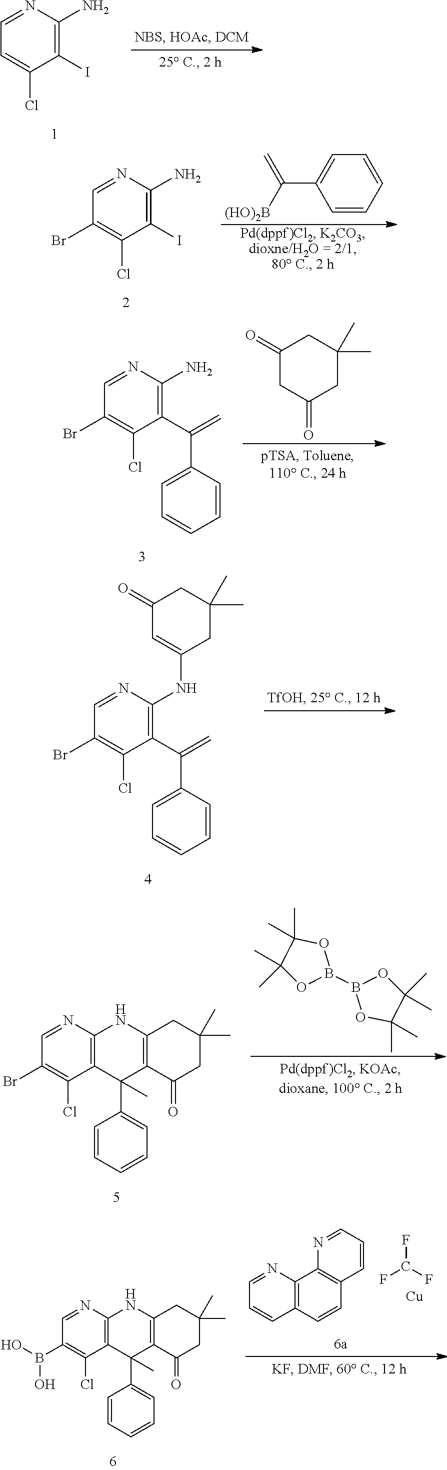

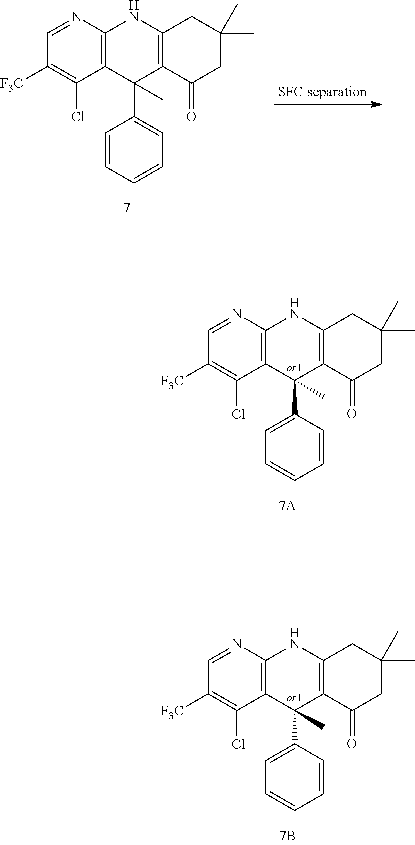 [SMILES: CC1(C)CC(=O)C2=C(C1)Nc1ncc(C(F)(F)F)c(Cl)c1C2(C)c1ccccc1 ; CC1(C)CC(=O)C2=C(C1)Nc1ncc(C(F)(F)F)c(Cl)c1[C@]2(C)c1ccccc1 ; CC1(C)CC(=O)C2=C(C1)Nc1ncc(C(F)(F)F)c(Cl)c1[C@@]2(C)c1ccccc1]

### Step 1: 5-bromo-4-chloro-3-iodopyridin-2-amine (2)

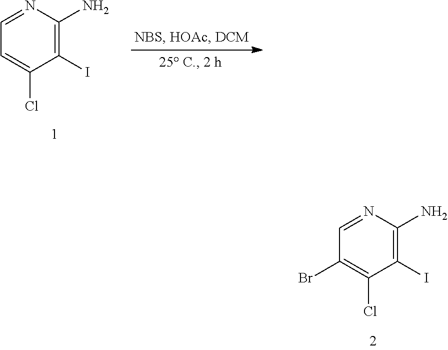 [SMILES: Nc1nccc(Cl)c1I ; Nc1ncc(Br)c(Cl)c1I]

To a solution of 4-chloro-3-iodo-pyridin-2-amine (13.00 g, 51.1 mmol, 1.00 eq) in HOAc (40 mL) and DCM (80 mL) was added NBS (9.89 g, 56.2 mmol, 1.10 eq) portion wise was stirred at 25° C. for 2 h. On completion, the reaction mixture was basified to pH=10 with NaOH (2M). The reaction mixture was diluted with DCM (200 mL) and the organic layer was washed with brine (100 mL), dried over Na₂SO₄, filtered, and concentrated under reduced pressure to give a residue. The yellow residue was purified by column chromatography (SiO₂, Petroleum ether/Ethyl acetate=3/1) to give compound 2 (15 g, 79% yield) as a yellow solid.

LCMS: tR=0.436 min., (ES⁺) m/z (M+H)⁺=332.8.

### Step 2: 5-bromo-4-chloro-3-(1-phenylvinyl)pyridin-2-amine (3)

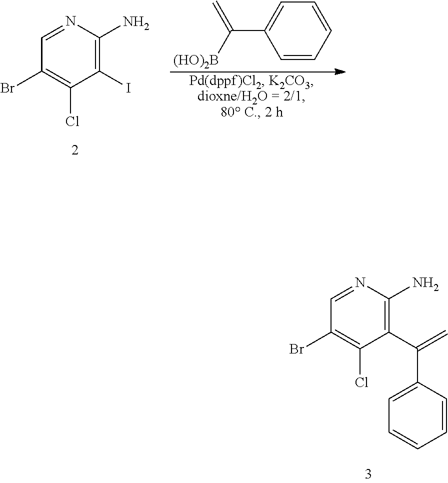

To a solution of 1-phenylvinylboronic acid (9.99 g, 67.5 mmol, 1.50 eq) and compound 2 (15.0) g, 45.0 mmol, 1.00 eq) in dioxane (150 mL) and H₂O (75 mL) was added K₂CO₃ (18.63 g, 135 mmol, 3.00 eq) and Pd(dppf)Cl₂ (3.29 g, 4.50 mmol, 0.100 eq) portion wise with the temperature kept at 25° C. under N₂ to get a yellow solution. The reaction mixture was stirred at 80° C. for 2 hours. On completion, the reaction mixture was quenched with saturated NH₄Cl (100 mL), and then extracted with EtOAc (150 mL×3). The combined organic layers were washed with brine (150 mL×2), dried over Na₂SO₄, filtered, and concentrated under reduced pressure to give a residue. The residue was purified by flash silica gel chromatography (ISCO®; 330 g SepaFlash® Silica Flash Column. Eluent of 0˜100% Ethyl acetate/Petroleum ether gradient @ 100 mL/min) to give compound 3 (10 g, 72% yield) as a red solid.

¹H NMR (400 MHz, DMSO-do) δ=8.17 (s, 1H), 7.35-7.30 (m, 5H), 6.14 (s, 1H), 6.05 (s, 2H), 5.35 (s, 1H).

### Step 3: 3-((5-bromo-4-chloro-3-(1-phenylvinyl)pyridin-2-yl)amino)-5,5-dimethylcyclohex-2-en-1-one (4)

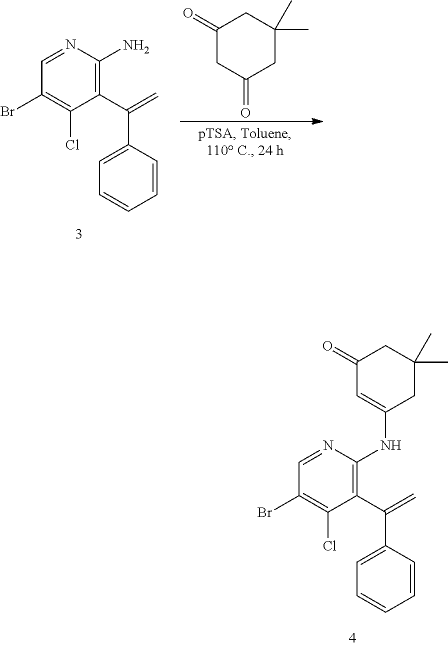

To a solution of 5,5-dimethylcyclohexane-1,3-dione (4.98 g, 35.5 mmol, 1.10 eq) and compound 3 (10.00 g, 32.3 mmol, 1.00 eq) in toluene (100 mL) was added pTSA (0.56 g, 3.23 mmol, 0.100 eq) and MgSO₄ (5.00 g, 41.7 mmol, 1.29 eq) portion wise with the temperature kept at 25° C. to get a yellow solution. The reaction mixture was stirred at 110° C. for 24 hours. On completion, the reaction mixture was quenched with H₂O (50 mL), and then extracted with EtOAc (50 mL×3). The combined organic layers were washed with brine (50 mL×3), dried over Na₂SO₄, filtered, and concentrated under reduced pressure to give a residue. The residue was purified by flash silica gel chromatography (ISCO®; 330 g SepaFlash® Silica Flash Column, Eluent of 0˜100% Ethyl acetate/Petroleum ether gradient @ 80 mL/min) to give compound 4 (11.00 g, 59% yield) as a yellow solid.

¹H NMR (400 MHz, DMSO-d₆) δ=8.66 (s, 1H), 7.90 (s, 1H), 7.35-7.30 (m, 3H), 7.30-7.26 (m, 2H), 6.34-6.25 (m, 2H), 5.75 (s, 1H), 5.51 (s, 1H), 2.09 (s, 1H), 2.05-1.96 (m, 3H), 0.87 (br d, J=18.4 Hz, 6H).

### Step 4: 3-bromo-4-chloro-5,8,8-trimethyl-5-phenyl-7,8,9,10-tetrahydrobenzo[b][1,8]naphthyridin-6(5H)-one (5)

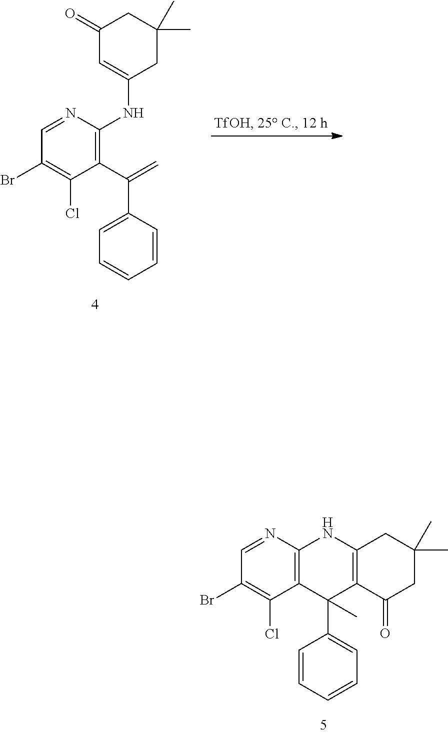 [SMILES: C=C(c1ccccc1)c1c(NC2=CC(=O)CC(C)(C)C2)ncc(Br)c1Cl ; CC1(C)CC(=O)C2=C(C1)Nc1ncc(Br)c(Cl)c1C2(C)c1ccccc1]

To a solution of compound 4 (11.00 g, 25.5 mmol, 1.00 eq) in TfOH (110 mL) was stirred at 25° C. for 12 hours. On completion, the reaction mixture was quenched with NaOH (2.5M, 100 mL), and then extracted with EtOAc (75 mL×3). The combined organic layers were washed with brine (75 mL×3), dried over Na₂SO₄, filtered, and concentrated under reduced pressure to give a residue. The residue was purified by flash silica gel chromatography (ISCO®; 330 g SepaFlash® Silica Flash Column, Eluent of 0˜100% Ethyl acetate/Petroleum ether gradient @ 60 mL/min) to give compound 5 (7.50 g, 68% yield) as a yellow solid.

¹H NMR (400 MHz, DMSO-d₆) δ=10.08 (br s, 1H), 8.34 (s, 1H), 7.32 (br d, J=7.6 Hz, 2H), 7.17 (br t, J=7.6 Hz, 2H), 7.07-7.01 (m, 1H), 2.42 (br s, 2H), 2.11 (s, 3H), 1.99 (d, J=3.2 Hz, 1H), 1.95 (s, 1H), 1.87-1.81 (m, 1H), 0.97 (s, 3H), 0.89 (s, 3H).

### Step 5: (4-chloro-5,8,8-trimethyl-6-oxo-5-phenyl-5,6,7,8,9,10-hexahydrobenzo[b][1,8]naphthyridin-3-yl)boronic acid (6)

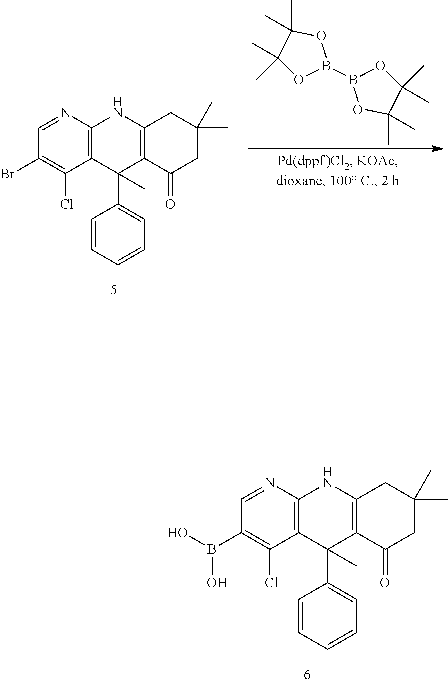

To a solution of compound 5 (6.50 g, 15.1 mmol, 1.00 eq) and 4,4,5,5-tetramethyl-2-(4,4,5,5-tetramethyl-1,3,2-dioxaborolan-2-yl)-1,3,2-dioxaborolane (3.82 g, 15.1 mmol, 1.00 eq) in dioxane (70 mL) was added KOAc (2.96 g, 30.2 mmol, 2.00 eq) and Pd(dppf)Cl₂ (1.085 g, 15.1 mmol, 0.1 00 eq) was stirred at 100° C. for 2 h. On completion, the mixture quenched by 70 mL H₂O and

extracted with EtOAc (50 mL×3). The combined organic layer was washed with brine (50 mL), dried over Na₂SO₄, filtered, and concentrated under reduced pressure. The yellow residue was purified by column chromatography (SiO₂, Ethyl acetate/Petroleum ether gradient=3/1) to give compound 6 (1.2 g, 20% yield) as a yellow solid.

LCMS: tR=0.540 min., (ES⁺) m/z (M+H)⁺=443.1.

### Step 6: (S)-4-chloro-5,8,8-trimethyl-5-phenyl-3-(trifluoromethyl)-5,8,9,10-tetrahydrobenzo [b] [1,8]naphthyridin-6(7H)-one (7A)

### (R)-4-chloro-5,8,8-trimethyl-5-phenyl-3-(trifluoromethyl)-5,8,9,10-tetrahydrobenzo[b][1,8] naphthyridin-6(7H)-one (7B)

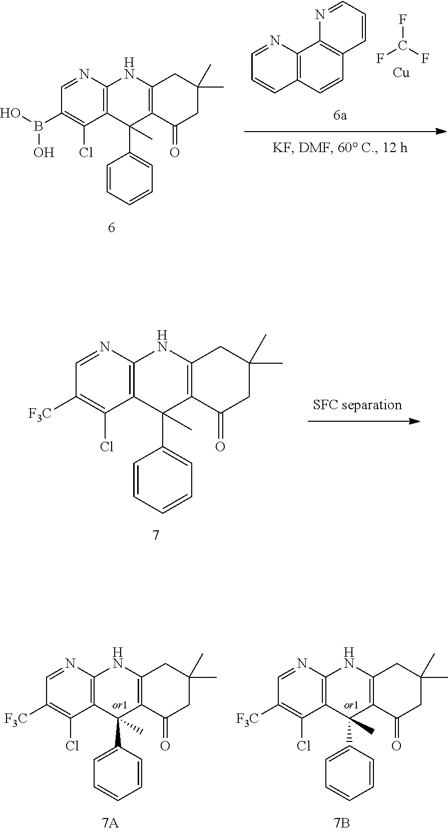

To a solution of compound 6 (100 mg, 0.252 mmol, 1.00 eq) and compound 6a (119 mg, 0.378 mmol, 1.50 eq) in DMF (1 mL) was added KF (15 mg, 0.252 mmol, 1.00 eq) was stirred at 60° C. for 2 h. On completion, the mixture quenched by 50 mL H₂O and the reaction mixture was diluted with EtOAc (50 mL). The organic layer was washed with brine (50 mL×3), dried over Na₂SO₄, filtered, and concentrated under reduced pressure to give a residue. The yellow residue was purified by column chromatography (SiO₂, Ethyl acetate/Petroleum ether gradient=3/1) to give compound 7 (120 mg, 23% yield) as a yellow solid.

The product 120 mg was separated by SFC (Chiralpak Whelk 50×4.6 mm I.D. 3.5 um); Mobile phase: IPA (0.05% DEA) in CO₂ from 5% to 40%; Flow rate: 3 mL/min Wavelength: 220 nm) to give:

Peak 1: (S)-4-chloro-5,8,8-trimethyl-5-phenyl-3-(trifluoromethyl)-5,8,9,10-tetrahydrobenzo [b][1,8]naphthyridin-6(7H)-one (7A). (29.0 mg, 24% yield).

LCMS: tR=0.516 min., (ES⁺) m/z (M+H)⁺=421.2.

¹H NMR (400 MHz, DMSO-d₆) δ=10.44 (s, 1H), 8.48 (s, 1H), 7.37 (br d, J=7.6 Hz, 2H), 7.24-7.20 (m, 2H), 7.11-7.06 (m, 1H), 2.48 (s, 2H), 2.17 (s, 3H), 2.01 (s, 1H), 1.93-1.88 (m, 1H), 1.27 (s, 1H), 1.02 (s, 3H), 0.93 (s, 3H).

Peak 2: (R)-4-chloro-5,8,8-trimethyl-5-phenyl-3-(trifluoromethyl)-5,8,9,10-tetrahydrobenzo[b][1,8]naphthyridin-6(7H)-one (7B). (27.5 mg, 23% yield).

LCMS: tR=0.516 min., (ES⁺) m/z (M+H)⁺=421.3.

¹H NMR (400 MHz, DMSO-d6) δ=10.43-10.40 (m, 1H), 8.45 (s, 1H), 7.34 (br d, J=7.6 Hz, 2H), 7.19 (t, J=8.0 Hz, 2H), 7.08-7.04 (m, 1H), 2.45 (s, 2H), 2.14 (s, 3H), 2.03-1.98 (m, 1H), 1.90-1.85 (m, 1H), 0.99 (s, 3H), 0.90 (s, 3H).

### Example 1.28. (S)-3-bromo-4-chloro-5,8,8-trimethyl-5-phenyl-5,8,9,10-tetrahydrobenzo [b][1,8]naphthyridin-6(7H)-one. (5A)

### (R)-3-bromo-4-chloro-5,8,8-trimethyl-5-phenyl-5,8,9,10-tetrahydrobenzo[b][1,8] naphthyridin-6(7H)-one. (5B)

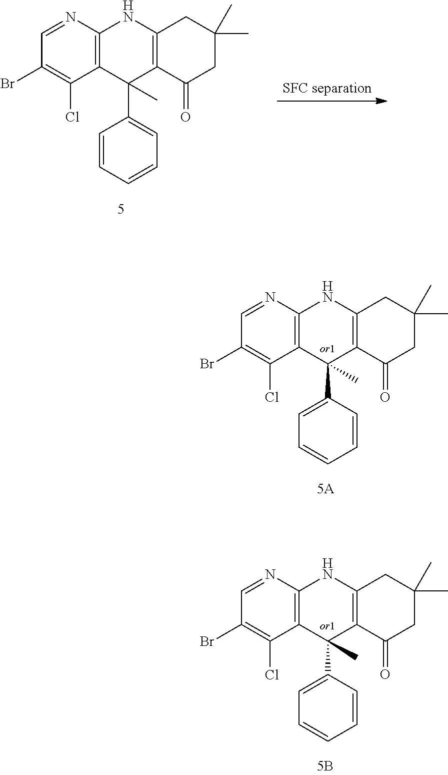 [SMILES: CC1(C)CC(=O)C2=C(C1)Nc1ncc(Br)c(Cl)c1C2(C)c1ccccc1 ; "SFC separation" ; CC1(C)CC(=O)C2=C(C1)Nc1ncc(Br)c(Cl)c1[C@]2(C)c1ccccc1 ; CC1(C)CC(=O)C2=C(C1)Nc1ncc(Br)c(Cl)c1[C@@]2(C)c1ccccc1]

The 200 mg compound 5 was separated by SFC (DAICEL CHIRALPAK AS (250 mm×30 mm, 10 um); Mobile phase: MeOH (0.01% NH₃H₂O) in CO₂ from 35% to 35%; Flow rate: 80 mL/min Wavelength: 220 nm) to give:

Peak 1: (S)-3-bromo-4-chloro-5,8,8-trimethyl-5-phenyl-5,8,9,10-tetrahydrobenzo [b][1,8] naphthyridin-6(7H)-one. (5A) (68 mg, 34% yield).

LCMS: tR=0.504 min., (ES⁺) m/z (M+H)⁺=431.1.

¹H NMR (400 MHz, DMSO-d6) δ=10.08 (s, 1H), 8.34 (s, 1H), 7.32 (d, J=7.4 Hz, 2H), 7.17 (t, J=7.8 Hz, 2H), 7.08-6.99 (m, 1H), 2.42 (d, J=2.0 Hz, 2H), 2.11 (s, 3H), 2.02-1.81 (m, 2H), 0.97 (s, 3H), 0.89 (s, 3H).

Peak 2: (R)-3-bromo-4-chloro-5,8,8-trimethyl-5-phenyl-5,8,9,10-tetrahydrobenzo [b][1,8] naphthyridin-6(7H)-one. (5B) (83 mg, 41% yield).

LCMS: tR=0.500 min., (ES⁺) m/z (M+H)⁺=431.1.

¹H NMR (400 MHz, DMSO-d₆) δ=10.08 (s, 1H), 8.34 (s, 1H), 7.32 (d, J=7.4 Hz, 2H), 7.17 (t, J=7.8 Hz, 2H), 7.07-7.01 (m, 1H), 2.42 (d, J=2.4 Hz, 2H), 2.11 (s, 3H), 2.01-1.81 (m, 2H), 0.98 (s, 3H), 0.89 (s, 3H).

### Example 1.29. (R)-3,4-difluoro-5,8,8-trimethyl-5-phenyl-5,8,9,10-tetrahydrobenzo[b][1,8]naphthyridin-6(7H)-one (5A)

### (S)-3,4-difluoro-5,8,8-trimethyl-5-phenyl-5,8,9,10-tetrahydrobenzo[b][1,8]naphthyridin-6(7H)-one (5B)

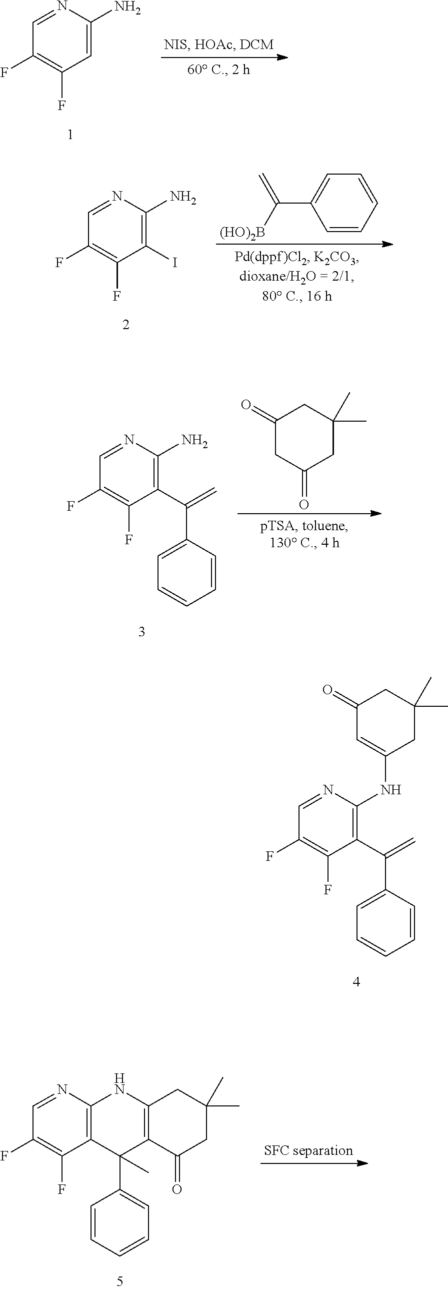

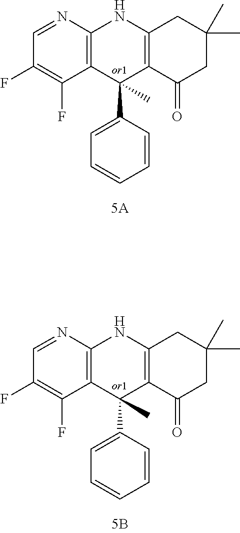 [SMILES: CC1(C)CC(=O)C2=C(C1)Nc1ncc(F)c(F)c1[C@]2(C)c1ccccc1 ; CC1(C)CC(=O)C2=C(C1)Nc1ncc(F)c(F)c1[C@@]2(C)c1ccccc1]

### Step 1: 4,5-difluoro-3-iodopyridin-2-amine (2)

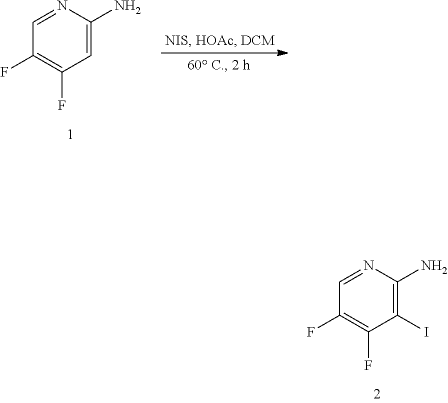 [SMILES: Nc1cc(F)c(F)cn1 ; Nc1ncc(F)c(F)c1I]

To a solution of 4,5-difluoropyridin-2-amine (100 mg, 768.67 μmol, 1 eq) in DCM (1 mL) and AcOH (0.5 mL) was added NIS (190.23 mg, 845.53 μmol, 1.1 eq) at 20° C. The mixture was stirred at 20° C. for 1 h. On completion, the reaction mixture was diluted with H₂O (10 mL) and extracted with EtOAc (20 mL×2). The combined organic layers were washed with NaHCO₃ (20 mL×2) and brine (20 mL×2), dried over Na₂SO₄, filtered, and concentrated under reduced pressure to give a residue. The residue was triturated from EtOAc (10 mL) at 25° C. for 30 min to give compound 2 (180 mg, crude) as a white solid.

¹H NMR (400 MHz, DMSO-d₆) δ=8.08-8.07 (d, J=4 Hz, 1H), 6.35 (br s, 2H), 6.18 (br s, 1H).

LCMS: tR=0.728 min., (ES⁺) m/z (M+H)⁺=256.8.

### Step 2: 4,5-difluoro-3-(1-phenylvinyl)pyridin-2-amine (3)

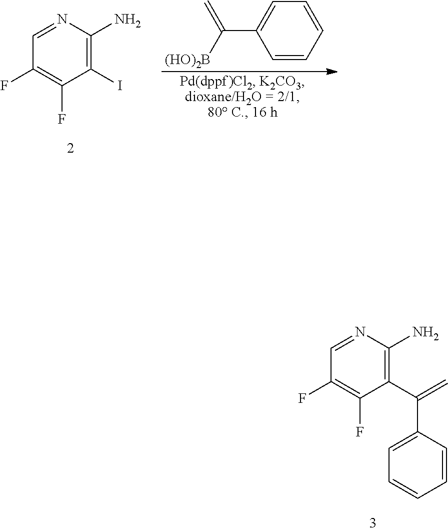

To a mixture of compound 2 (1.23 g, 4.80 mmol, 1 eq), 1-phenylvinylboronic acid (710.24 mg,

4.80 mmol, 1 eq) and K₂CO₃ (1.33 g, 9.60 mmol, 2 eq) in dioxane (15 mL) and H₂O (5 mL) was added Pd(dppf)Cl₂·CH₂Cl₂ (391 mg, 480 μmol, 0.1 eq) under N₂. The mixture was stirred at 80° C. for 1.5 h. On completion, the reaction mixture was concentrated under reduced pressure. The residue was diluted with H₂O (30 mL) and extracted with EtOAc (20 mL×2). The combined organic layers were washed with H₂O (10 mL) and brine, dried over Na₂SO₄, filtered, and concentrated under reduced pressure to give a residue. The residue was purified by flash silica gel chromatography (ISCO®; 20 g SepaFlash® Silica Flash Column, Eluent of 0˜15% Ethyl acetate/Petroleum ether gradient @ 80 mL/min) to give compound 3 (700 mg, 45% yield) as a white solid.

LCMS: tR=0.555 min., (ES⁺) m/z (M+H)⁺=256.8.

### Step 3: 3-((4,5-difluoro-3-(1-phenylvinyl)pyridin-2-yl)amino)-5,5-dimethylcyclohex-2-en-1-one (4)

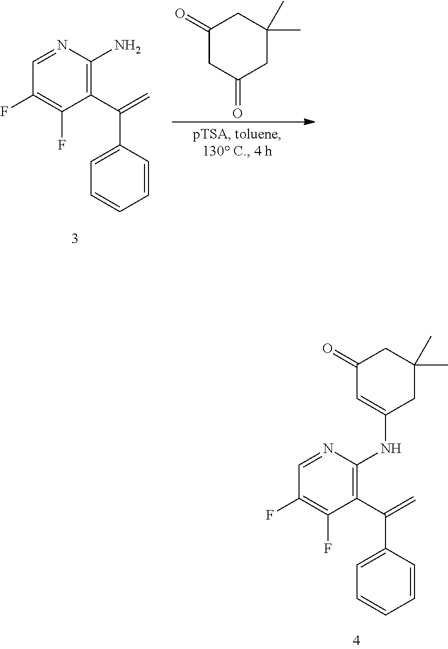

To a solution of compound 3 (500 mg, 2.15 mmol, 1 eq) in toluene (9 mL) was added 5,5-dimethylcyclohexane-1,3-dione (362.18 mg, 2.58 mmol, 1.2 eq), MgSO₄ (1.04 g, 8.61 mmol, 4 eq) and pTSA (37.08 mg, 215.31 μmol, 0.1 eq). The mixture was stirred at 130° C. for 4 h. On completion, the reaction mixture was diluted with H₂O (30 mL) and extracted with EtOAc (25 mL×2). The combined organic layers were washed with brine (10 mL×2), dried over Na₂SO₄, filtered, and concentrated under reduced pressure to give a residue. The residue was purified by flash silica gel chromatography (ISCO®; 20 g SepaFlash® Silica Flash Column, Eluent of 0˜20% Ethyl acetate/Petroleum ether gradient @ 80 mL/min) to give the compound 4 (600 mg, 78% yield) as a brown solid.

LCMS: tR=1.841 min., (ES⁺) m/z (M+H)⁺=355.1.

### Step 4: (R)-3,4-difluoro-5,8,8-trimethyl-5-phenyl-5,8,9,10-tetrahydrobenzo[b][1,8]naphthyridin-6(7H)-one (5A) & (S)-3,4-difluoro-5,8,8-trimethyl-5-phenyl-5,8,9,10-tetrahydrobenzo[b][1,8]naphthyridin-6(7H)-one (5B)

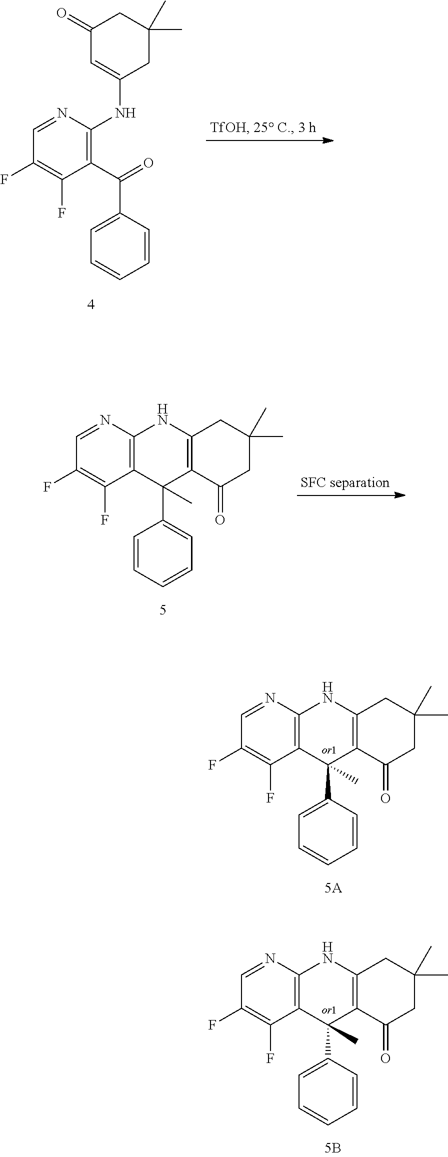 [SMILES: CC1(C)CC(=O)C=C(Nc2ncc(F)c(F)c2C(=O)c2ccccc2)C1 ; CC1(C)CC(=O)C2=C(C1)Nc1ncc(F)c(F)c1C2(C)c1ccccc1 ; CC1(C)CC(=O)C2=C(C1)Nc1ncc(F)c(F)c1[C@]2(C)c1ccccc1 ; CC1(C)CC(=O)C2=C(C1)Nc1ncc(F)c(F)c1[C@@]2(C)c1ccccc1]

To a solution of compound 4 (900 mg, 2.54 mmol, 1 eq) in TfOH (10 mL) was stirred at 20° C. for 12 h. The reaction mixture was diluted with ice H₂O (50 mL) and adjusted to pH=10 with 1N NaOH. Then extracted with EtOAc (20 mL×2). The combined organic layers were washed with brine (10 mL×2), dried over, filtered, and concentrated under reduced pressure to give a residue. The residue was purified by flash silica gel chromatography (ISCO®; 12 g SepaFlash® Silica Flash Column, Eluent of 0˜20% Ethyl acetate/Petroleum ether gradient @ 80 mL/min) to give the compound 5 (860 mg, 90% yield) as a yellow solid.

The product 260 mg was separated by SFC (column: Chiralpak AS-3 50×4.6 mm; mobile phase: [0.05% DEA MeOH]; B %: 5%-40%, 3 mL/min) to give:

Peak 1: (R)-3,4-difluoro-5,8,8-trimethyl-5-phenyl-7,8,9,10-tetrahydrobenzo[b][1,8]naphthyridin-6(5H)-one. (5A) (68.2 mg, 26% yield).

LCMS: tR=0.492 min., (ES⁺) m/z (M+H)⁺=355.0.

¹H NMR (400 MHz, DMSO-do) δ=10.07 (s, 1H), 8.23 (d, J=8.8 Hz, 1H), 7.35 (d, J=7.6 Hz, 2H), 7.22 (t, J=7.6 Hz, 2H), 7.09-7.04 (m, 1H), 2.45 (s, 2H), 2.04-1.99 (m, 4H), 1.91-1.86 (m, 11H), 1.00-0.93 (m, 6H).

Peak 2: (S)-3,4-difluoro-5,8,8-trimethyl-5-phenyl-7,8,9,10-tetrahydrobenzo[b][1,8]naphthyridin-6(5H)-one. (5B) (67.8 mg, 26% yield).

LCMS: tR=0.498 min., (ES⁺) m/z (M+H)⁺=355.1.

¹H NMR (400 MHz, DMSO-d6) δ=10.07 (s, 1H), 8.23 (d, J=8.0 Hz, 1H), 7.35 (d, J=7.6 Hz, 2H), 7.22 (t, J=7.6 Hz, 2H), 7.09-7.05 (m, 1H), 2.45 (s, 2H), 2.04-1.99 (m, 4H), 1.91-1.86 (m, 1H), 1.00-0.94 (m, 6H).

### Example 1.30. (S)-5,8,8-trimethyl-6-oxo-5-phenyl-3-(trifluoromethyl)-5,6,7,8,9,10-hexahydrobenzo[b][1,8]naphthyridine-4-carbonitrile (9A)

### (R)-5,8,8-trimethyl-6-oxo-5-phenyl-3-(trifluoromethyl)-5,6,7,8,9,10-hexahydrobenzo[b][1,8]naphthyridine-4-carbonitrile (9B)

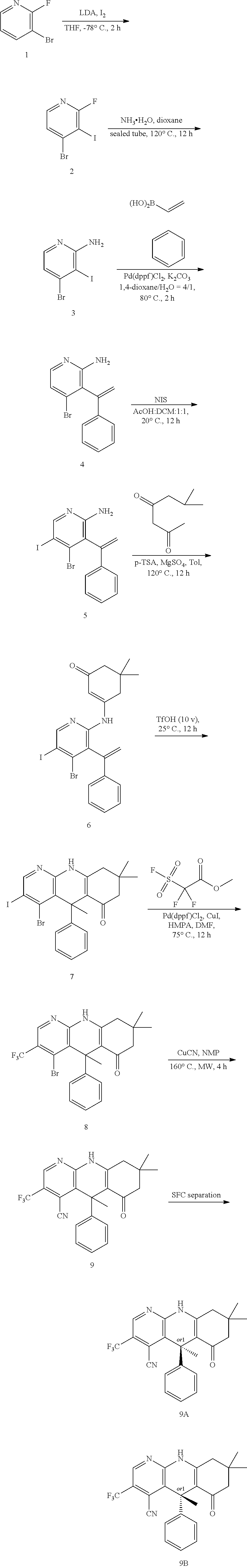

### Step 1: 4-bromo-2-fluoro-3-iodopyridine (2)

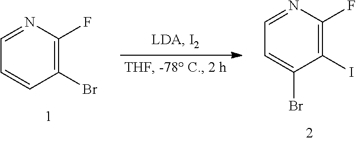 [SMILES: Fc1ncccc1Br ; Fc1nccc(Br)c1I]

To a solution of compound 1 (50.00 g, 284 mmol, 1.00 eq) in THF (503.88 mL) was added LDA (2 M in THF, 156.26 mL, 313 mmol, 1.10 eq) dropwise stirred under N₂ at −70° C. for 2 h, then the mixture was stirred 12 (108.24 g, 426 mmol, 1.50 eq) under N₂ at −70° C. for 1 h. On completion, the reaction mixture was quenched with saturated NH₄Cl (600 mL) at 20° C. and extracted with EtOAc (3×600 mL), washed with saturated NaCl (900 mL), dried over Na₂SO₄, filtered, and concentrated under reduced pressure to give a residue. The residue was purified by flash silica gel chromatography (ISCO®; 330 g SepaFlash® Silica Flash Column, Eluent of 4˜10% Ethyl acetate/Petroleum ether gradient @ 80 mL/min) to give compound 2 (70.00 g, 82% yield) as a yellow solid.

¹H NMR (400 MHz, DMSO-d6) δ=8.08 (d, J=5.2 Hz, 1H), 7.69 (dd, J=0.8, 5.2 Hz, 1H).

### Step 2: 4-bromo-3-iodopyridin-2-amine (3)

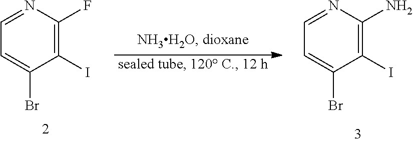

To a solution of compound 2 (60.00 g, 199 mmol, 1.00 eq) in 1,4-dioxane (735 mL) was added NH₃·H₂O (138.02 mL, 994 mmol, 5.00 eq) stirred at 120° C. for 12 h in a pressure kier. On completion, the reaction was diluted with water (1000 mL), extracted with EtOAc (3×1000 mL), washed with saturated NaCl (1500 mL), dried over Na₂SO₄, filtered, and concentrated under reduced pressure to give a residue. The residue was purified by flash silica gel chromatography (ISCO®; 330 g SepaFlash® Silica Flash Column, Eluent of 4˜8% Ethyl acetate/Petroleum ether gradient @ 80 mL/min) to give compound 3 (49.50 g, 83% yield) as a yellow solid.

¹H NMR (400 MHz, DMSO-d6) δ=7.76 (d, J=5.2 Hz, 1H), 6.83 (d, J=5.2 Hz, 1H), 6.41 (br s, 2H).

### Step 3: 4-bromo-3-(1-phenylvinyl)pyridin-2-amine (4)

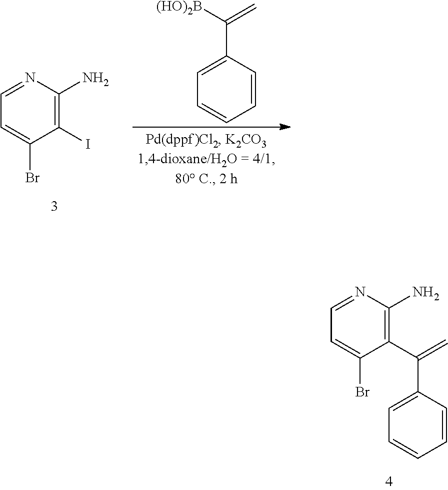 [SMILES: Nc1nccc(Br)c1I ; C=C(B(O)O)c1ccccc1 ; C=C(c1ccccc1)c1c(Br)ccnc1N]

To a solution of compound 3 (31.00 g, 104 mmol, 1.00 eq) and 1-phenylvinylboronic acid (18.42 g, 124 mmol, 1.20 eq) in 1,4-dioxane (280 mL) and H₂O (70 mL) was added K₂CO₃ (43.00 g, 311 mmol, 3.00 eq) and Pd(dppf)Cl₂ CH₂Cl₂ (8.47 g, 10.4 mmol, 0.100 eq) in one portion stirred under N₂ at 80° C. for 2 h. On completion, the reaction was diluted with water (400 mL), extracted with EtOAc (3×400 mL), washed with saturated NaCl (600 mL), dried over Na₂SO₄, filtered, and concentrated under reduced pressure to give a residue. The residue was purified by flash silica gel chromatography (ISCO®; 120 g SepaFlash® Silica Flash Column, Eluent of 18˜25% Ethyl acetate/Petroleum ether gradient @ 100 mL/min) to give compound 4 (24.00 g, 84% yield) as a yellow solid.

¹H NMR (400 MHz, DMSO-d6) δ=7.81 (d, J=5.2 Hz, 1H), 7.36-7.28 (m, 5H), 6.87 (d, J=5.2 Hz, 1H), 6.11 (s, 1H), 5.83 (s, 2H), 5.30 (s, 1H).

### Step 4: 4-bromo-5-iodo-3-(1-phenylvinyl)pyridin-2-amine (5)

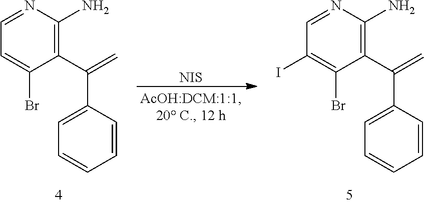

To a solution of compound 4 (24.00 g, 87.2 mmol, 1.00 eq) in AcOH (40 mL) and DCM (40 mL) was stirred at 20° C. for 12 h. On completion, the reaction was basified with NaHCO₃ to pH=9, the residue was diluted with water (100 mL), extracted with EtOAc (3×100 mL), washed with saturated Na₂S₂O₃ (150 mL), washed with saturated NaCl (150 mL), dried over Na₂SO₄, filtered, and concentrated under reduced pressure to give a residue. The residue was purified by flash silica gel chromatography (ISCO®; 330 g SepaFlash® Silica Flash Column, Eluent of 22˜35% Ethyl acetate/Petroleum ether gradient @ 100 mL/min) to give compound 5 (31.30 g, 89% yield) as a yellow solid.

¹H NMR (400 MHz, DMSO-d6) δ=8.26 (s, 1H), 7.35-7.33 (m, 2H), 7.32 (s, 3H), 6.07 (s, 1H), 6.01 (br s, 2H), 5.29 (s, 1H).

### Step 5: 3-((4-bromo-5-iodo-3-(1-phenylvinyl)pyridin-2-yl)amino)-5,5-dimethylcyclohex-2-en-1-one (6)

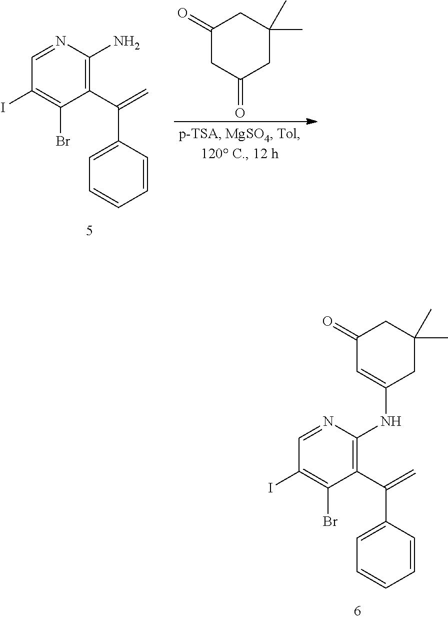

To a solution of compound 5 (31.30 g, 78.0 mmol, 1.00 eq) and 5,5-dimethylcyclohexane-1,3-dione (12.03 g, 85.8 mmol, 1.10 eq) in toluene (313 mL) was added p-TSA·H₂O (1.48 g, 7.80 mmol, 0.100 eq) and MgSO₄ (37.44 g, 312 mmol, 4.00 eq) in one portion stirred at 120° C. for 12 h. On completion, the reaction was diluted with water (300 mL), extracted with EtOAc (3×300 mL), washed with saturated NaCl (500 mL), dried over Na₂SO₄, filtered, and concentrated under reduced pressure to give a residue. The residue was purified by flash silica gel chromatography (ISCO®; 330 g SepaFlash® Silica Flash Column, Eluent of 45˜58% Ethyl acetate/Petroleum ether gradient @ 100 mL/min) to give compound 6 (18.30 g, 45% yield) as a yellow solid.

¹H NMR (400 MHz, DMSO-d6) δ=8.73 (s, 1H), 7.80 (s, 1H), 7.36-7.29 (m, 4H), 7.28-7.25 (m, 2H), 6.33 (s, 1H), 6.20 (s, 1H), 5.44 (s, 1H), 2.29-2.20 (m, 2H), 2.00 (s, 2H), 0.89 (s, 3H), 0.84 (s, 3H).

### Step 6: 4-bromo-3-iodo-5,8,8-trimethyl-5-phenyl-5,8,9,10-tetrahydrobenzo[b][1,8]naphthyridin-6(7H)-one (7)

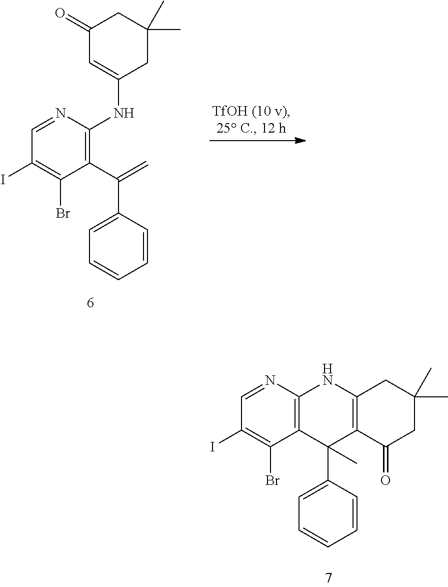 [SMILES: C=C(c1ccccc1)c1c(NC2=CC(=O)CC(C)(C)C2)ncc(I)c1Br ; CC1(C)CC(=O)C2=C(C1)Nc1ncc(I)c(Br)c1C2(C)c1ccccc1]

To a solution of compound 6 (12.00 g, 22.9 mmol, 1.00 eq) in TfOH (120 mL) was stirred at 25° C. for 12 h. On completion, the reaction was basified with NaOH to pH=9, the residue was diluted with water (200 mL), extracted with EtOAc (3×200 mL), washed with saturated NaCl (300 mL), dried over Na₂SO₄, filtered, and concentrated under reduced pressure to give a residue. The residue was purified by flash silica gel chromatography (ISCO®; 120 g SepaFlash® Silica Flash Column, Eluent of 11˜15% Ethyl acetate/Petroleum ether gradient @ 100 mL/min) to give compound 7 (9.30 g, 78% yield) as a yellow solid.

¹H NMR (400 MHz, DMSO-d6) δ=10.01 (s, 1H), 8.44 (s, 1H), 7.30 (br d, J=8.4 Hz, 2H), 7.14 (t, J=7.6 Hz, 2H), 7.05-6.99 (m, 1H), 2.40 (br d, J=3.2 Hz, 2H), 2.14 (s, 3H), 1.99-1.92 (m, 1H), 1.86-1.79 (m, 1H), 0.96 (s, 3H), 0.86 (s, 3H).

### Step 7: 4-bromo-5,8,8-trimethyl-5-phenyl-3-(trifluoromethyl)-5,8,9,10-tetrahydrobenzo[b][1,8]naphthyridin-6(7H)-one (8)

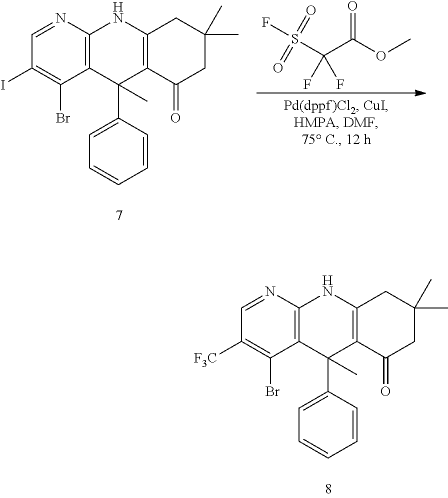

To a solution of compound 7 (3.00 g, 5.73 mmol, 1.00 eq) in DMF (15 mL) was added HMPA (3.08 g, 17.2 mmol, 3.00 eq), Pd(dppf)Cl₂CH₂Cl₂ (464 mg, 0.57 mmol, 0.100 eq) and CuI (2.18 g, 11.5 mmol, 2.00 eq) in one portion stirred under N₂ at 75° C. for 1.5 h. Then a solution of methyl 2,2-difluoro-2-fluorosulfonyl-acetate (5.51 g, 28.7 mmol, 5.00 eq) in DMF (15 mL) was added dropwise to the mixture, the mixture was stirred under N₂ at 75° C. for 16 h. On completion, the reaction was diluted with water (50 mL), extracted with EtOAc (3×50 mL), washed with NH₃·H₂O (90 mL), washed with saturated NaCl (90 mL), dried over Na₂SO₄, filtered, and concentrated under reduced pressure to give a residue. The crude product was purified by reversed phase to give compound 8 (1.80 g, 67% yield) as a yellow solid.

### Step 8: (S)-5,8,8-trimethyl-6-oxo-5-phenyl-3-(trifluoromethyl)-5,6,7,8,9,10-hexahydrobenzo[b][1,8]naphthyridine-4-carbonitrile (9A) & (R)-5,8,8-trimethyl-6-oxo-5-phenyl-3-(trifluoromethyl)-5,6,7,8,9,10-hexahydrobenzo[b][1,8]naphthyridine-4-carbonitrile (9B)

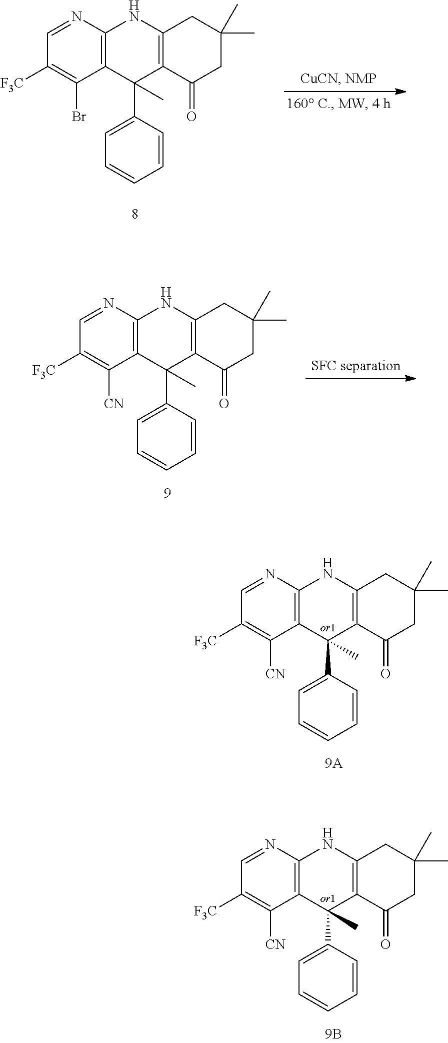 [SMILES: CC1(C)CC(=O)C2=C(C1)Nc1ncc(C(F)(F)F)c(Br)c1C2(C)c1ccccc1 ; CC1(C)CC(=O)C2=C(C1)Nc1ncc(C(F)(F)F)c(C#N)c1C2(C)c1ccccc1 ; CC1(C)CC(=O)C2=C(C1)Nc1ncc(C(F)(F)F)c(C#N)c1[C@]2(C)c1ccccc1 ; CC1(C)CC(=O)C2=C(C1)Nc1ncc(C(F)(F)F)c(C#N)c1[C@@]2(C)c1ccccc1]

To a solution of compound 8 (200 mg, 0.430 mmol, 1.00 eq) in NMP (2 mL) was added CuCN (191 mg, 2.15 mmol, 5.00 eq) stirred at 160° C. for 4 h. On completion, the reaction was diluted with water (10 mL), extracted with EtOAc (3×10 mL), washed with NH₃—H₂O (15 mL) washed with saturated NaCl (15 mL), dried over Na₂SO₄, filtered, and concentrated under reduced pressure to give a residue. The residue was purified by prep-HPLC to give 100 mg product. 100 mg compound 9 was separated by SFC to give:

Peak 1: (S)-5,8,8-trimethyl-6-oxo-5-phenyl-3-(trifluoromethyl)-5,6,7,8,9,10-hexahydrobenzo [b][1,8]naphthyridine-4-carbonitrile (9A). (35 mg, 20% yield).

LCMS: tR=0.466 min., (ES⁺) m/z (M+H)⁺=412.6.

¹H NMR (400 MHz, DMSO-d6) δ=10.68 (s, 1H), 8.61 (s, 1H), 7.35 (br d, J=7.6 Hz, 2H), 7.23 (t, J=7.6 Hz, 2H), 7.16-7.06 (m, 1H), 2.47 (br s, 2H), 2.18 (s, 3H), 2.05-1.98 (m, 1H), 1.94-1.87 (m, 1H), 0.99 (s, 3H), 0.91 (s, 3H).

Peak 2: (R)-5,8,8-trimethyl-6-oxo-5-phenyl-3-(trifluoromethyl)-5,6,7,8,9,10-hexahydrobenzo [b][1,8]naphthyridine-4-carbonitrile (9B). (34 mg, 18% yield).

LCMS: tR=0.470 min., (ES⁺) m/z (M+H)⁺=412.7.

¹H NMR (400 MHz, DMSO-d6) δ=10.68 (s, 1H), 8.61 (s, 1H), 7.35 (br d, J=7.6 Hz, 2H), 7.23 (t, J=7.6 Hz, 2H), 7.14-7.08 (m, 1H), 2.47 (s, 2H), 2.18 (s, 3H), 2.04-1.98 (m, 1H), 1.93-1.87 (m, 1H), 0.99 (s, 3H), 0.91 (s, 3H).

### Example 1.31. (S)-3-fluoro-5,8,8-trimethyl-5-(3-(methylsulfonyl)phenyl)-4-(trifluoromethyl)-7,8,9,10-tetrahydrobenzo[b][1,8]naphthyridin-6(5H)-one (11A)

### (R)-3-fluoro-5,8,8-trimethyl-5-(3-(methylsulfonyl)phenyl)-4-(trifluoromethyl)-7,8,9,10-tetrahydrobenzo[b][1,8]naphthyridin-6(5H)-one (11B)

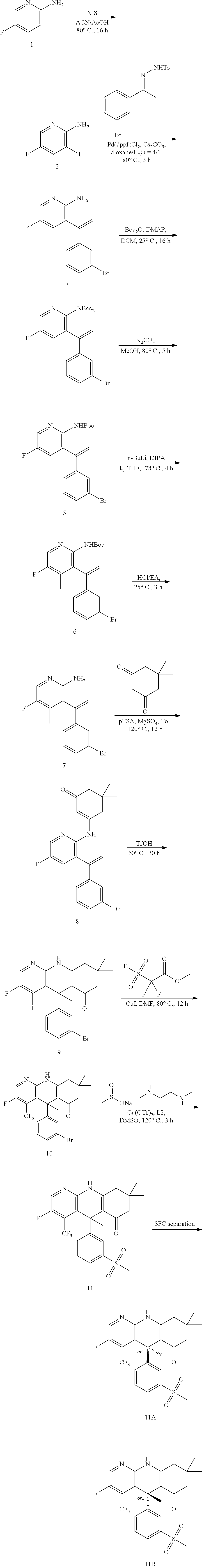

### Step 1: 5-fluoro-3-iodopyridin-2-amine (2)

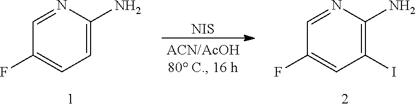 [SMILES: Nc1ccc(F)cn1 ; "NIS" ; Nc1ncc(F)cc1I]

To a solution of 5-fluoropyridin-2-amine (20.0 g, 178 mmol, 1.00 eq) in ACN (200 mL) and AcOH (200 mL) was added NIS (60.2 g, 268 mmol, 1.50 eq) portion wise to the reaction mixture. The mixture was stirred at 80° C. for 16 h. On completion, the reaction mixture was adjusted to pH=9 with Na₂CO₃ solution. The reaction mixture was diluted with EtOAc (3×200 mL). The organic layer was washed with brine (2×200 mL), dried over Na₂SO₄, filtered, and concentrated in vacuo. The residue was purified by flash silica gel chromatography (ISCO®; 330 g SepaFlash® Silica Flash Column, Eluent of 0˜10% Ethyl acetate/Petroleum ether gradient @ 100 mL/min) to give compound 2 (18 g, 42% yield) as a yellow solid.

LCMS: tR=0.248 min., (ES⁺) m/z (M+H)⁺=238.9.

### Step 2: 3-(1-(3-bromophenyl)vinyl)-5-fluoropyridin-2-amine (3)

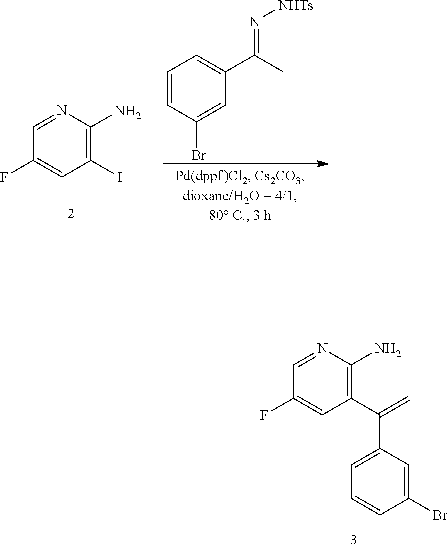

To a solution of compound 2 (14.0 g, 58.8 mmol, 1.00 eq) (E)-N′-(1-(3-bromophenyl)ethylidene)-4-methylbenzenesulfonohydrazide (19.4 g, 52.9 mmol, 0.9 eq) and Cs₂CO₃ (57.3 g, 176 mmol, 3.00 eq) in dioxane (160 mL) and H₂O (40 mL) was added Pd(dppf)Cl₂ (4.67 g, 5.88 mmol, 0.1 eq) in one portion to the reaction mixture under N₂. The reaction mixture was stirred at 80° C. for 3 h. On completion, the reaction mixture was diluted with EtOAc (3×200 mL). The organic layer was washed with brine (2×200 mL), dried over Na₂SO₄, filtered, and concentrated in vacuo. The residue was purified by flash silica gel chromatography (ISCO®; 220 g SepaFlash® Silica Flash Column, Eluent of 0˜10% Ethyl acetate/Petroleum ether gradient @ 100 mL/min) to give compound 3 (10 g, 62% yield) as a yellow solid.

LCMS: tR=0.314 min., (ES⁺) m/z (M+H)⁺=294.0.

### Step 3: tert-butyl N-[3-[1-(3-bromophenyl)vinyl]-5-fluoro-2-pyridyl]-N-tert-butoxycarbonyl-carbamate (4)

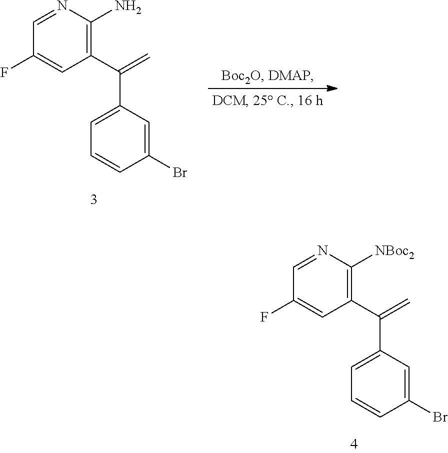

To a solution of compound 3 (10.0 g, 34.1 mmol, 1.00 eq) and DMAP (345 mg, 3.41 mmol, 0.10 eq) in DCM (100 mL) was added Boc₂O (22.3 g, 102 mmol, 3.00 eq) portion wise to the reaction mixture at 25° C. The reaction mixture was stirred at 25° C. for 16 h. On completion, the reaction mixture was diluted with EtOAc (3×200 mL). The organic layer was washed with brine (2×200 mL), dried over Na₂SO₄, filtered, and concentrated in vacuo. The residue was purified by flash silica gel chromatography (ISCO®; 120 g SepaFlash® Silica Flash Column, Eluent of 0˜10% Ethyl acetate/Petroleum ether gradient @ 100 mL/min) to give compound 5 (13.0 g, 77% yield) as a yellow solid.

### Step 4: tert-butyl (3(1-(3-bromophenyl)vinyl)-5-fluoropyridin-2-yl)carbamate (5)

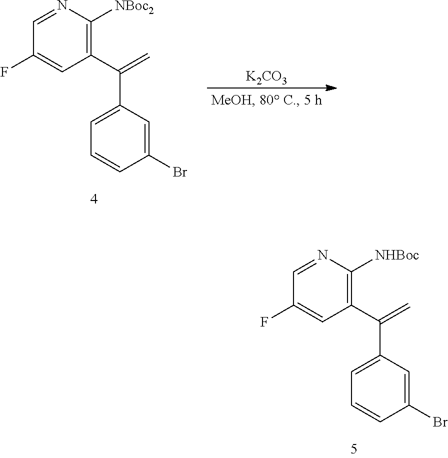

To a solution of compound 4 (1.30 g, 2.63 mmol, 1.00 eq) in MeOH (20 mL) was added K₂CO₃ (1.09 g, 7.90 mmol, 3.00 eq) portion wise to the reaction mixture. The reaction mixture was stirred at 80° C. for 5 h. On completion, the reaction mixture was diluted with EtOAc (3×50 mL). The organic layer was washed with brine (2×50 mL), dried over Na₂SO₄, filtered, and concentrated in vacuo. The residue was purified by flash silica gel chromatography (ISCO®; 10 g SepaFlash® Silica Flash Column, Eluent of 0˜10% Ethyl acetate/Petroleum ether gradient @ 100 mL/min) to give compound 5 (500 mg, 48% yield) as a yellow solid.

### Step 5: tert-butyl (3-(1-(3-bromophenyl)vinyl)-5-fluoro-4-iodopyridin-2-yl)carbamate (6)

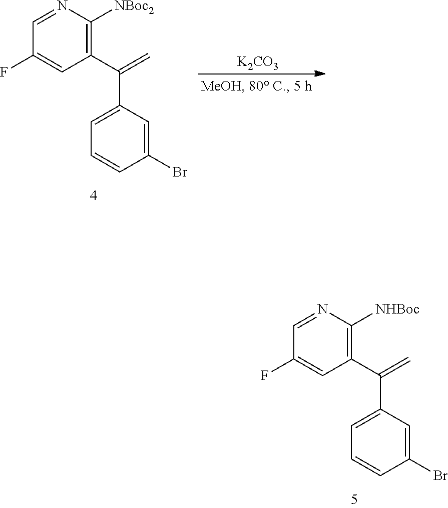

To a solution of DIPA (1.05 g, 9.66 mmol, 1.00 eq) in THF (40 mL) was added n-BuLi (618 mg, 9.66 mmol, 1.00 eq) dropwise to the reaction mixture at −78° C. under N₂, The reaction mixture was stirred at −78° C. for 0.5 h, Then was added compound 5 (3.80 g, 9.66 mmol, 1.00 eq) in THF (40 mL) dropwise to the reaction mixture, The reaction mixture was stirred at −78° C. for 2 h, Then was added I₂ (2.5 g, 9.66 mmol, 1.00 eq) in THF (10 mL) dropwise to the reaction mixture, The reaction mixture was stirred at −78° C. for 2 h. On completion, the mixture quenched by 20 ml NH₄Cl. The reaction mixture was diluted with EtOAc (3×100 mL). The organic layer was washed with brine (2×100 mL), dried over Na₂SO₄, filtered, and concentrated in vacuo. The residue was purified by flash silica gel chromatography (ISCO®; 40 g SepaFlash® Silica Flash Column, Eluent of 0˜10% Ethyl acetate/Petroleum ether gradient @ 60 mL/min) to give compound 6 (4.30 g, 86% yield) as a white solid.

LCMS: tR=0.499 min., (ES⁺) m/z (M+H)⁺=463.0.

### Step 6: 3-(1-(3-bromophenyl)vinyl)-5-fluoro-4-iodopyridin-2-amine (7)

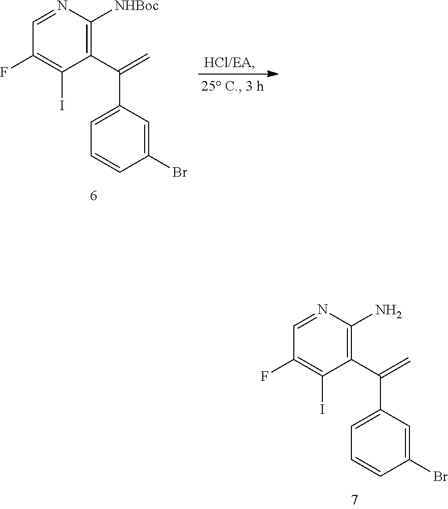 [SMILES: C=C(c1cccc(Br)c1)c1c(NC(=O)OC(C)(C)C)ncc(F)c1I ; "HCl/EA," ; C=C(c1cccc(Br)c1)c1c(N)ncc(F)c1I]

The reaction mixture of compound 6 (1.60 g, 3.08 mmol, 1.00 eq) in HCl/EA (16 mL) was stirred at 25° C. for 3 h. On completion, the mixture was filtered, and the filter cake was dried under vacuum. The mixture was basified to pH=7 with Na₂CO₃. The reaction mixture was diluted with EtOAc (3×100 mL). The organic layer was washed with brine (2×100 mL), dried over Na₂SO₄, filtered, and concentrated in vacuo. The residue was purified by flash silica gel chromatography (ISCO®; 10 g SepaFlash® Silica Flash Column, Eluent of 0˜10% Ethyl acetate/Petroleum ether gradient @ 60 mL/min) to give compound 7 (1.30 g, 89% yield) as a white solid.

LCMS: tR=0.438 min., (ES⁺) m/z (M+H)⁺=429.0.

### Step 7: 3-((3-(1-(3-bromophenyl)vinyl)-5-fluoro-4-iodopyridin-2-yl)amino)-5,5-dimethylcyclohex-2-enone (8)

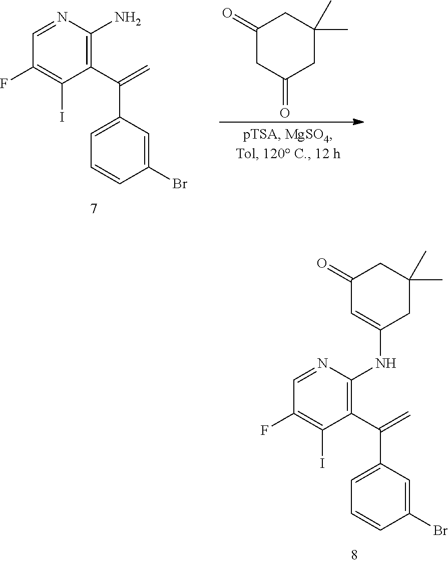

The reaction mixture of compound 7 (1.90 g, 4.53 mmol, 1.00 eq) 5,5-dimethylcyclohexane-1,3-dione (763 mg, 5.44 mmol, 1.20 eq) MgSO₄ (544 mg, 4.53 mmol, 1.00 eq) and pTSA (866 mg, 4.53 mmol, 1.00 eq) in toluene (20 mL) was stirred at 120° C. for 12 h. On completion, the reaction mixture was filtered and concentrated in vacuo, the reaction mixture was diluted with EtOAc (3×100 mL). The organic layer was washed with brine (2×100 mL), dried over Na₂SO₄, filtered, and concentrated in vacuo. The residue was purified by flash silica gel chromatography (ISCO®; 60 g SepaFlash® Silica Flash Column, Eluent of 0˜10% Ethyl acetate/Petroleum ether gradient @ 60 mL/min) to give compound 8 (2.10 g, 86% yield) as a yellow solid.

LCMS: tR=0.505 min., (ES⁺) m/z (M+H)⁺=540.9.

### Step 8: 5-(3-bromophenyl)-3-fluoro-4-iodo-5,8,8-trimethyl-7,8,9,10-tetrahydrobenzo[b][1,8]naphthyridin-6(5H)-one (9)

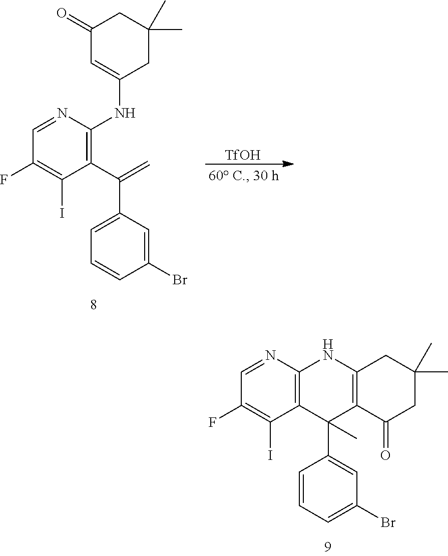

To a solution of compound 8 (1.40 g, 2.59 mmol, 1.00 eq) in TfOH (7.7 g, 51.7 mmol, 20.0 eq) was stirred at 60° C. for 30 h. On completion, the mixture quenched by 20 ml H₂O and basified to pH=9 with Na₂CO₃. The reaction mixture was diluted with EtOAc (3×100 mL). The organic layer was washed with brine (2×100 mL), dried over Na₂SO₄, filtered, and concentrated in vacuo. The residue was purified by flash silica gel chromatography (ISCO®; 40 g SepaFlash® Silica Flash Column, Eluent of 0˜10% Ethyl acetate/Petroleum ether gradient @ 60 mL/min) to give compound 9 (1.40 g, 100% yield) as a white solid.

### Step 9: 5-(3-bromophenyl)-3-fluoro-4-iodo-5,8,8-trimethyl-7,8,9,10-tetrahydrobenzo[b][1,8] naphthyridin-6(5H)-one (10)

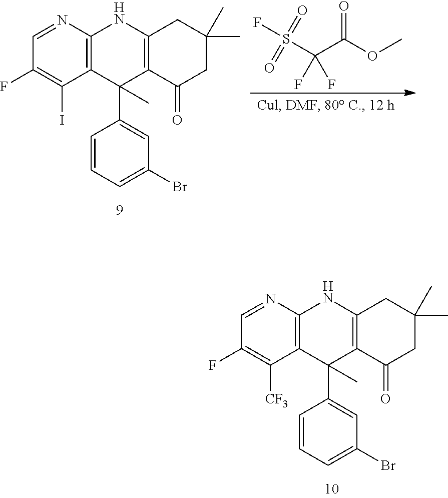

To a solution of compound 9 (100 mg, 0.09 mmol, 1.00 eq) CuI (34 mg, 0.09 mmol, 1.00 eq) in DMF (3 mL) was added methyl 2,2-difluoro-2-fluorosulfonyl-acetate (178 mg, 0.46 mmol, 5.0 eq) portion wise to the reaction mixture. The reaction mixture was stirred at 80° C. for 12 h. On completion, the reaction mixture was poured into saturated NH₄·H₂O aqueous solution (5 mL). The reaction mixture was diluted with EtOAc (3×20 mL). The organic layer was washed with brine (2×20 mL), dried over Na₂SO₄, filtered, and concentrated in vacuo. The residue was purified by flash silica gel chromatography (ISCO®; 40 g SepaFlash® Silica Flash Column, Eluent of 0˜10% Ethyl acetate/Petroleum ether gradient @ 60 mL/min) to give compound 10 (1.40 g, 100% yield) as a white solid.

LCMS: tR=1.785 min., (ES⁺) m/z (M+H)⁺=484.9.

### Step 10: (S)-3-fluoro-5,8,8-trimethyl-5-(3-(methylsulfonyl)phenyl)-4-(trifluoromethyl)-7,8,9,10-tetrahydrobenzo[b][1,8]naphthyridin-6(5H)-one (11A)

### (R)-3-fluoro-5,8,8-trimethyl-5-(3-(methylsulfonyl)phenyl)-4-(trifluoromethyl)-7,8,9,10-tetrahydrobenzo[b][1,8]naphthyridin-6(5H)-one (11B)

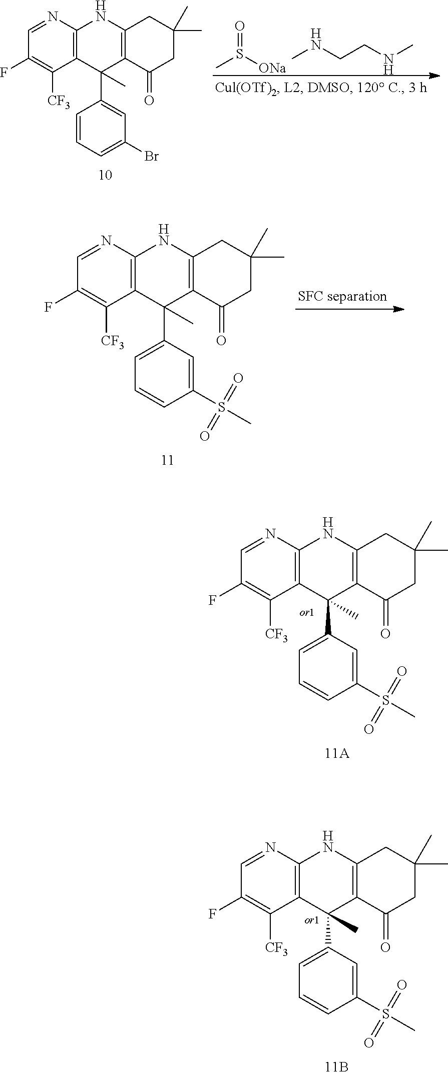

To a solution of compound 10 (150 mg, 0.02 mmol, 1.00 eq) bis(trifluoromethylsulfonyloxy) copper (118 mg, 0.02 mmol, 1.0) eq) methylsulfinyloxysodium (4.5 mg, 0.04 mmol, 2.00 eq) in DMSO (5 mL) was added N,N′-dimethylethane-1,2-diamine (4.1 mg, 0.04 mmol, 2.10 eq) dropwise to the reaction mixture. The reaction mixture was stirred at 120° C. for 3 h. On completion, the reaction mixture was diluted with EtOAc (3×20 mL). The organic layer was washed with brine (2×20 mL), dried over Na₂SO₄, filtered, and concentrated in vacuo. The residue was purified by prep-HPLC to give compound 11 (50 mg, 33% yield) as a white solid.

LCMS: tR=1.481 min., (ES⁺) m/z (M+H)⁺=483.0.

The product 27 mg was separated by SFC ((Method-B\AD-3-IPA (DEA)-5-40-3 mL-35T.l cm) Column: Chiralpak AD-3 50×4.6 mm I.D., 3 um, Mobile phase: Phase A for CO₂, and Phase B for IPA (0.05% DEA); Gradient elution: B in A from 5% to 40%, Flow rate:
3 mL/min; Detector: PDA; Column Temp: 35 C; Back Pressure: 100 Bar) to give:

Peak 1: (S)-3-fluoro-5,8,8-trimethyl-5-(3-(methylsulfonyl)phenyl)-4-(trifluoromethyl)-7,8,9,10-tetrahydrobenzo[b][1,8]naphthyridin-6(5H)-one (11A). (13 mg, 49% yield).

LCMS: tR=0.436 min., (ES⁺) m/z (M+H)⁺=483.0.

H NMR (400 MHz, DMSO-d₆) δ=10.30 (s, 1H), 8.51 (s, 1H), 7.83 (br d, J=1.6 Hz, 1H), 7.63 (br d, J=7.6 Hz, 2H), 7.49-7.39 (m, 1H), 3.08 (s, 3H), 2.52 (s, 2H), 2.42 (br d, J=4.4 Hz, 2H), 2.09 (s, 2H), 1.94 (s, 1H), 1.88-1.81 (m, 1H), 0.96 (s, 3H), 0.82 (s, 3H).

Peak 2: (R)-3-fluoro-5,8,8-trimethyl-5-(3-(methylsulfonyl)phenyl)-4-(trifluoromethyl)-7,8,9,10-tetrahydrobenzo[b][1,8]naphthyridin-6(5H)-one (11A). (13 mg, 49% yield).

LCMS: tR=0.436 min., (ES⁺) nm/z (M+H)⁺=483.0.

¹H NMR (400 MHz, DMSO-d₆) δ=10.29 (s, 1H), 8.51 (d, J=1.6 Hz, 1H), 7.83 (br s, 1H), 7.70-7.56 (m, 2H), 7.50-7.39 (m, 1H), 3.08 (s, 3H), 2.42 (br d, J=4.8 Hz, 2H), 2.09 (s, 3H), 1.94 (s, 1H), 1.90-1.78 (m, 1H), 0.96 (s, 3H), 0.82 (s, 3H).

### Example 1.32. (5S)-3-fluoro-5,8,8-trimethyl-5-(3-methylsulfonylphenyl)-6-oxo-9,10-dihydro-7H-benzo[b][1,8]naphthyridine-4-carbonitrile (3A)

### (5R)fluoro-5,8,8-trimethyl-5-(3-methylsulfonylphenyl)-6-oxo-9,10-dihydro-7H-benzo[b][1,8]napthyridine-4-carbonitrile (3B)

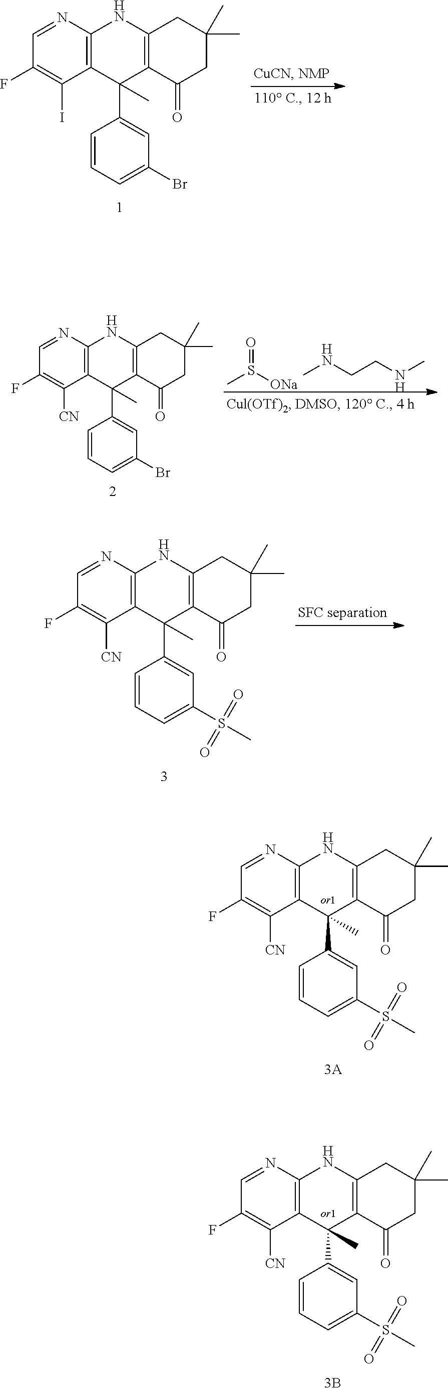

### Step 1: 5-(3-bromophenyl)-3-fluoro-5,8,8-trimethyl-6-oxo-9,10-dihydro-7H-benzo[b][1,8] naphthyridine-4-carbonitrile (2)

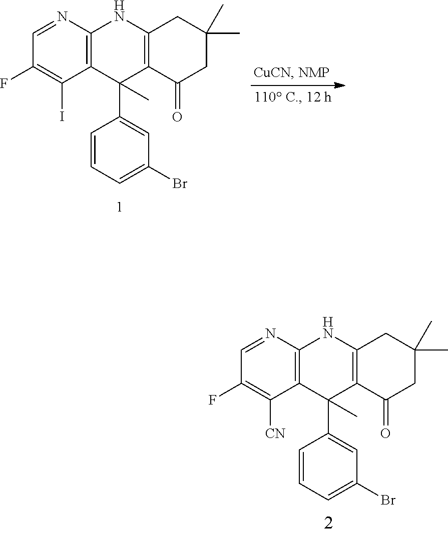 [SMILES: CC1(C)CC(=O)C2=C(C1)Nc1ncc(F)c(I)c1C2(C)c1cccc(Br)c1 ; CC1(C)CC(=O)C2=C(C1)Nc1ncc(F)c(C#N)c1C2(C)c1cccc(Br)c1]

To a solution of 1 (400 mg, 0.74 mmol, 1.00 eq) in NMP (4 mL) was added CuCN (78 mg, 0.89 mmol, 1.20 eq) portion wise with the temperature kept at 25° C. under N₂ to get a yellow solution. The reaction mixture was stirred at 110° C. for 12 h. On completion, the reaction mixture was quenched with H₂O (5 mL), and then extracted with EtOAc (5 mL×3). The combined organic layers were washed with brine (5 mL×3), dried over Na2SO4, filtered, and concentrated under reduced pressure to give a residue. The residue was purified by flash silica gel chromatography (ISCO®; 10 g SepaFlash® Silica Flash Column. Eluent of 0˜100% Ethyl acetate/Petroleum ether gradient @ 60 mL/min) to give compound 2 (300 mg, 92% yield) as a yellow solid.

LCMS: tR=0.502 min., (ES⁺) m/z (M+H)⁺=440.1.

¹H NMR (400 MHz, DMSO-d₆) δ=10.32 (s, 1H), 8.43 (s, 1H), 7.56 (s, 1H), 7.35-7.29 (m, 2H), 7.23-7.17 (m, 1H), 2.46 (s, 2H), 2.12-2.06 (m, 3H), 2.00-1.98 (m, 1H), 1.93 (s, 1H), 0.99 (s, 3H), 0.92 (s, 3H).

### Step 3: (5S)-3-fluoro-5,8,8-trimethyl-5-(3-methylsulfonylphenyl)-6-oxo-9,10-dihydro-7H-benzo[b][1,8]naphthyridine-4-carbonitrile (3A)

### (5R)-3-fluoro-5,8,8-trimethyl-5-(3-methylsulfonylphenyl)-6-oxo-9,10-dihydro-7H-benzo[b][1,8]naphthyridine-4-carbonitrile (3B)

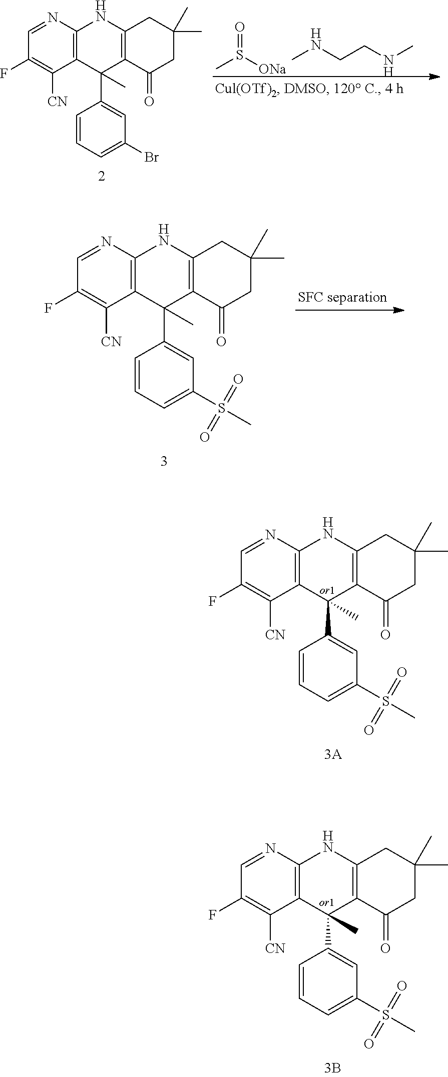

To a solution of methylsulfinyloxysodium (37 mg, 0.363 mmol, 2.00 eq) and Cu(OTf)2 (66 mg, 0.182 mmol, 1.00 eq) in DMSO (1 mL) was added N,N′-dimethylethane-1,2-diamine (34 mg, 0.382 mmol, 2.10 eq) and compound 2 (80 mg, 0.18 mmol, 1.00 eq) portion wise with the temperature kept at 25° C. under N₂. The reaction mixture was stirred at 120° C. for 4 h. On completion, the reaction mixture was poured into ammonia water (20 mL) and extracted with EtOAc (20 mL×3). The combined organic layer was washed with brine (20 mL), dried over Na₂SO₄, filtered, and concentrated. The residue was purified by prep-HPLC (column: Welch Xtimate C18 150×25 mm×5 um; mobile phase: [water (0.05% FA)-ACN]; B %: 30%-60%, 8 min) to give compound 3 (60 mg, 98% purity, 74% yield) as a yellow solid.

The product (60 mg) was separated by SFC (Column: Chiralpak AS-3 50×4.6 mm I.D., 3 um; Mobile phase: Phase A for CO₂, and Phase B for IPA (0.05% DEA); Gradient elution: B in A from 5% to 40%) to give:

Peak 1: (5S)-3-fluoro-5,8,8-trimethyl-5-(3-methylsulfonylphenyl)-6-oxo-9,10-dihydro-7H-benzo[b][1,8]naphthyridine-4-carbonitrile (3A). (8.6 mg, 14% yield).

LCMS: tR=0.417 min., (ES⁺) m/z (M+H)⁺=440.2.

¹H NMR (400 MHz, DMSO-d₆) δ=10.39 (s, 1H), 8.45 (s, 1H), 7.97 (s, 1H), 7.70 (t, J=8.0 Hz, 2H), 7.56-7.49 (m, 1H), 3.10 (s, 3H), 2.47 (s, 2H), 2.14 (s, 3H), 2.05-1.99 (m, 1H), 1.93-1.86 (m, 1H), 0.99 (s, 3H), 0.93 (s, 3H).

Peak 2: (5R)-3-fluoro-5,8,8-tri methyl-5-(3-methylsulfonylphenyl)-6-oxo-9,0-dihydro-7H-benzo[b][1,8]naphthyridine-4-carbonitrile (3B). (8.7 mg, 14% yield).

LCMS: tR=0.424 min., (ES⁺) m/z (M+H)⁺=440.1.

¹H NMR (400 MHz, DMSO-d₆) δ=10.39 (s, 1H), 8.45 (s, 1H), 7.97 (s, 1H), 7.70 (t, J=7.6 Hz, 2H), 7.55-7.49 (m, 1H), 3.10 (s, 3H), 2.48 (s, 2H), 2.14 (s, 3H), 2.04-1.99 (m, 1H), 1.92-1.85 (m, 1H), 0.99 (s, 3H), 0.92 (s, 3H).

### Example 1.33. (S)-3-fluoro-4,5,8,8-tetramethyl-5-(3-(methylsulfonyl)phenyl)-7,8,9,10-tetrahydrobenzo[b][1,8]naphthyridin-6(5H)-one (6A)

### (R)-3-fluoro-4,5,8,8-tetramethyl-5-(3-(methylsulfonyl)phenyl)-7,8,9,10-tetrahydrobenzo[b][1,8]naphthyridin-6(5H)-one (6B)

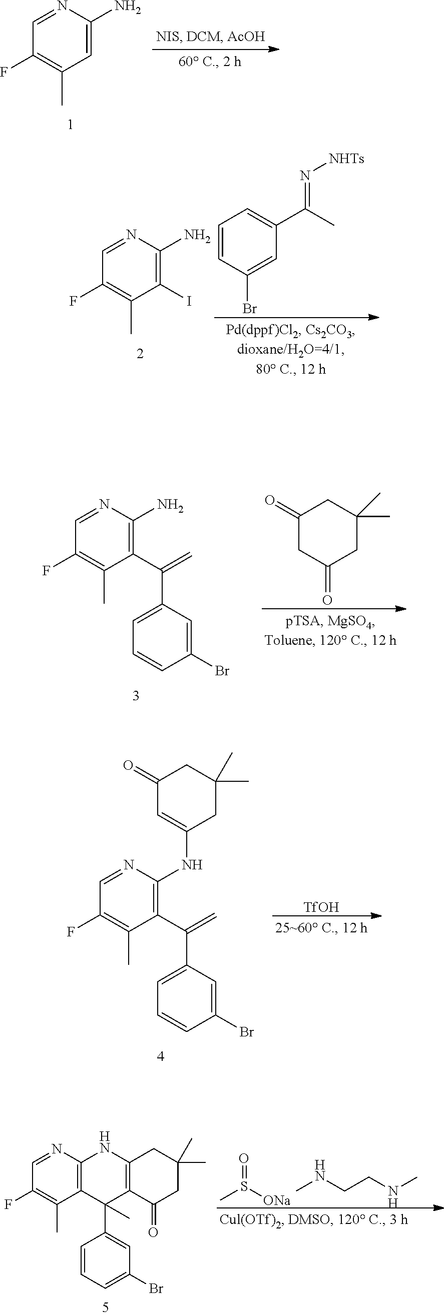

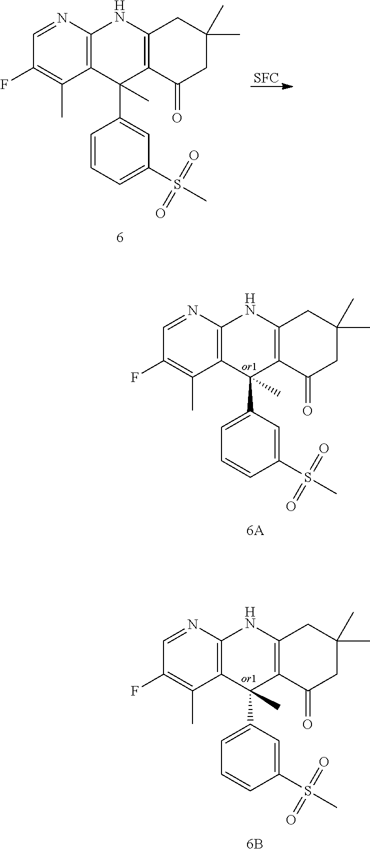

### Step 1: 5-fluoro-3-iodo-4-methylpyridin-2-amine (2)

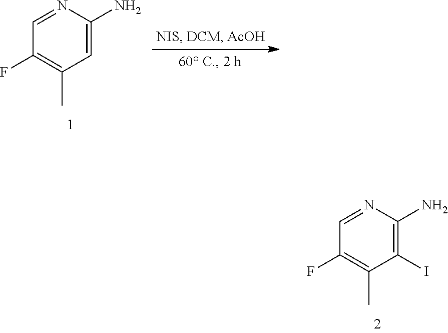

To a solution of 5-fluoro-4-methylpyridin-2-amine (2.00 g, 15.9 mmol, 1.00 eq) in DCM (16 mL) and AcOH (8 mL) was added NIS (5.3 mg, 23.8 mmol, 1.50 eq) portion wise to the reaction mixture, The reaction mixture was stirred at 60° C. for 2 h. On completion, the reaction mixture was diluted with EtOAc (3×100 mL). The organic layer was washed with brine (2×80 mL), dried over Na₂SO₄, filtered, and concentrated in vacuo. The residue was purified by flash silica gel chromatography (ISCO®; 40 g SepaFlash® Silica Flash Column. Eluent of 0˜10% Ethyl acetate/Petroleum ether gradient @ 100 mL/min) to give compound 2 (2.20 g, 55% yield) as a yellow solid.

LCMS: tR=0.261 min., (ES⁺) m/z (M+H)⁺=253.0.

### Step 2: 3-(1-(3-bromophenyl)vinyl)-5-fluoro-4-methylpyridin-2-amine (3)

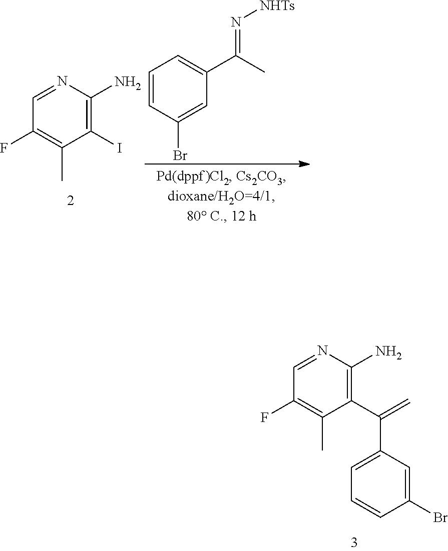

To a solution of compound 2 (2.20 g, 8.73 mmol, 1.00 eq)N-[(E)-1-(3-bromophenyl)ethylideneamino]-4-methyl-benzenesulfonamide (3.85 g, 10.5 mmol, 1.20 eq) and K₂CO₃ (3.6 mg, 26.2 mmol, 3.00 eq) in dioxane (20 mL) and H₂O (5 mL) was added Pd(dppf)Cl₂ (638 mg, 0.87 mmol, 0.10 eq) in one portion to the reaction mixture under N₂. The reaction mixture was stirred at 80° C. for 12 h. On completion, the reaction mixture was diluted with EtOAc (3×20 mL). The organic layer was washed with brine (3×20 mL), dried over Na₂SO₄, filtered, and concentrated in vacuo. The residue was purified by flash silica gel chromatography (ISCO®; 10 g SepaFlash® Silica Flash Column, Eluent of 0˜10% Ethyl acetate/Petroleum ether gradient @ 60 mL/min) to give compound 3 (2.0 g, 75% yield) as a yellow solid.

LCMS: tR=0.354 min., (ES⁺) m/z (M+H)⁺=307.0.

### Step 3: 3-((3-(1-(3-bromophenyl)vinyl)-5-fluoro-4-methylpyridin-2-yl)amino)-5,5-dimethylcyclohex-2-enone 4)

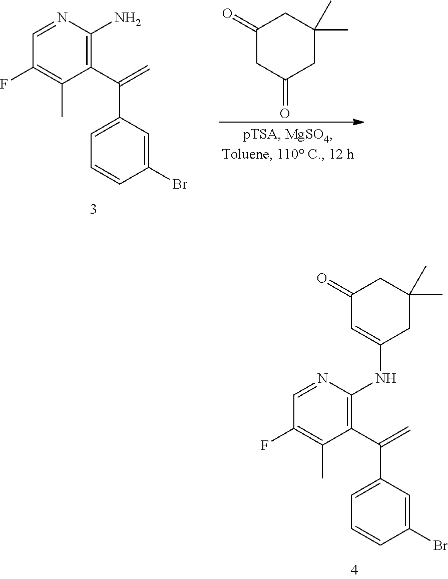

The reaction mixture of compound 3 (1.78 g, 5.81 mmol, 1.00 eq) 5,5-dimethylcyclohexane-1,3-dione (977 mg, 6.97 mmol, 1.20 eq) Mg₂SO₄ (3.3 g, 23.2 mmol, 4.00 eq) and pTSA (111 mg, 0.58 mmol, 0.10 eq) in toluene (30 mL) was stirred at 110° C. for 12 h. On completion, the reaction mixture was diluted with EtOAc (3×50 mL). The organic layer was washed with brine (3×50 mL), dried over Na₂SO₄, filtered, and concentrated in vacuo. The residue was purified by flash silica gel chromatography (ISCO®; 10 g SepaFlash® Silica Flash Column, Eluent of 0˜10% Ethyl acetate/Petroleum ether gradient (60 mL/min) to give compound (2 g, 79% yield) as a yellow solid.

LCMS: tR=0.492 min., (ES⁺) m/z (M+H)⁺=431.0.

### Step 4: 3-((3-(1-(3-bromophenyl)vinyl)-5-fluoro-4-methylpyridin-2-yl)amino)-5,5-dimethylcyclohex-2-enone (5)

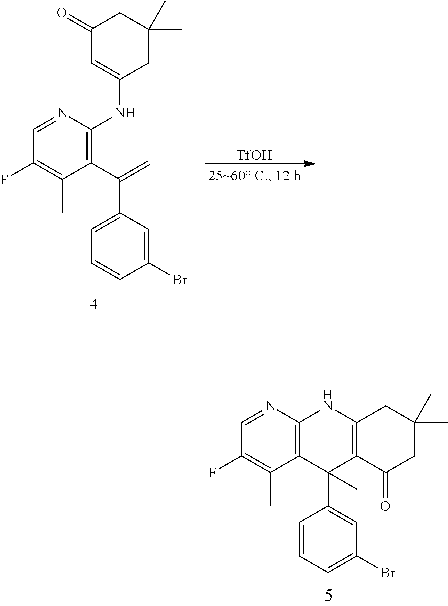 [SMILES: C=C(c1cccc(Br)c1)c1c(NC2=CC(=O)CC(C)(C)C2)ncc(F)c1C ; "TfOH" ; Cc1c(F)cnc2c1C(C)(c1cccc(Br)c1)C1=C(CC(C)(C)CC1=O)N2]

To a solution of compound 4 (1 g, 0.56 mmol) in TfOH (10 mL) was stirred at 25° C. for 12 h. On completion, the mixture was basified to pH=7 with Na₂CO₃. The reaction mixture was diluted with EtOAc (3×30 mL). The organic layer was washed with brine (2×30 mL), dried over Na₂SO₄, filtered, and concentrated in vacuo. The residue was purified by flash silica gel chromatography (ISCO®; 10 g SepaFlash® Silica Flash Column, Eluent of 0˜10% Ethyl acetate/Petroleum ether gradient @ 60 mL/min) to give compound 5 (700 mg 70% yield) as a yellow solid.

### Step 5: (S)-3-fluoro-4,5,8,8-tetramethyl-5-(3-(methylsulfonyl)phenyl)-7,8,9,10-tetrahydrobenzo [b][1,8]naphthyridin-6(5H)-one (6A)

### (R)-3-fluoro-4,5,8,8-tetramethyl-5-(3-(methylsulfonyl)phenyl)-7,8,9,10-tetrahydrobenzo[b][1,8] naphthyridin-6(5H)-one (6B)

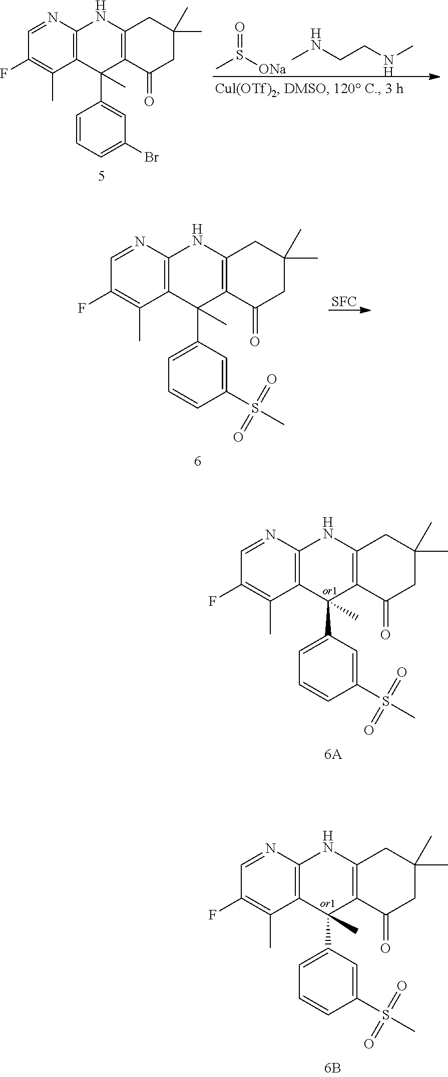

To a solution of compound 5 (350 mg, 0.02 mmol, 1.00 eq), bis(trifluoromethylsulfonyloxy) copper (294 mg, 0.02 mmol, 1.0) eq) methylsulfinyloxysodium (166 mg, 1.63 mmol, 2.00 eq) in DMSO (6 mL) was added N,N′-dimethylethane-1,2-diamine (143 mg, 1.63 mmol, 2.10 eq) dropwise to the reaction mixture. The reaction mixture was stirred at 120° C. for 3 h. On completion, the reaction mixture was diluted with EtOAc (3×20 mL). The organic layer was washed with brine (2×20 mL), dried over Na₂SO₄, filtered, and concentrated in vacuo. The residue was purified by prep-HPLC (column: Welch Xtimate C18 150×25 mm×5 um; mobile phase: [water (0.05% FA)-ACN]; B %: 24%-54%, 10 min) to give compound 6 (150 mg, 33% yield) as a white solid.

The product 150 mg was separated by SFC ((D:\Method-B\IC-3-MeOH (DEA)-5-40-3 mL-35T.l cm) Column: Chiralpak IC-3 50×4.6 mm I.D., 3 um Mobile phase: Phase A for CO₂, and Phase B for MeOH (0.05% DEA); Gradient elution: B in A from 5% to 40% Flow rate: 3 mL/min; Detector: PDA; Column Temp: 35 C; Back Pressure: 100 Bar) to give:

Peak 1: (S)-3-fluoro-4,5,8,8-tetramethyl-5-(3-(methylsulfonyl)phenyl)-7,8,9,10-tetrahydrobenzo [b][1,8]naphthyridin-6(5H)-one. (6A) (26.4 mg, 17% yield).

LCMS: tR=0.436 min., (ES⁺) m/z (M+H)⁺=455.3.

¹H NMR (400 MHz, DMSO-d₆) δ=9.89 (s, 1H), 8.05 (s, 1H), 7.88 (br s, 1H), 7.77-7.60 (m, 2H), 7.54-7.45 (m, 1H), 3.13 (s, 3H), 2.43 (s, 2H), 2.02 (s, 3H), 1.99-1.93 (m, 1H), 1.88-1.80 (m, 1H), 1.55 (d, J=2.4 Hz, 3H), 0.97 (s, 3H), 0.88 (s, 3H).

Peak 2: (R)-3-fluoro-4,5,8,8-tetramethyl-5-(3-(methylsulfonyl)phenyl)-7,8,9,10-tetrahydrobenzo [b][1,8]naphthyridin-6(5H)-one. (6B) (35 mg, 23% yield).

LCMS: tR=0.436 min., (ES⁺) m/z (M+H)⁺=455.3.

¹H NMR (400 MHz, DMSO-d6) δ=9.89 (s, 1H), 8.05 (s, 1H), 7.88 (br s, 1H), 7.77-7.60 (m, 2H), 7.54-7.43 (m, 1H), 3.13 (s, 3H), 2.43 (s, 2H), 2.02 (s, 3H), 1.99-1.93 (m, 1H), 1.88-1.80 (m, 1H), 1.55 (d, J=2.4 Hz, 3H), 0.97 (s, 3H), 0.88 (s, 3H).

### Example 1.34. (S)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (3A)

### (R)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (3B)

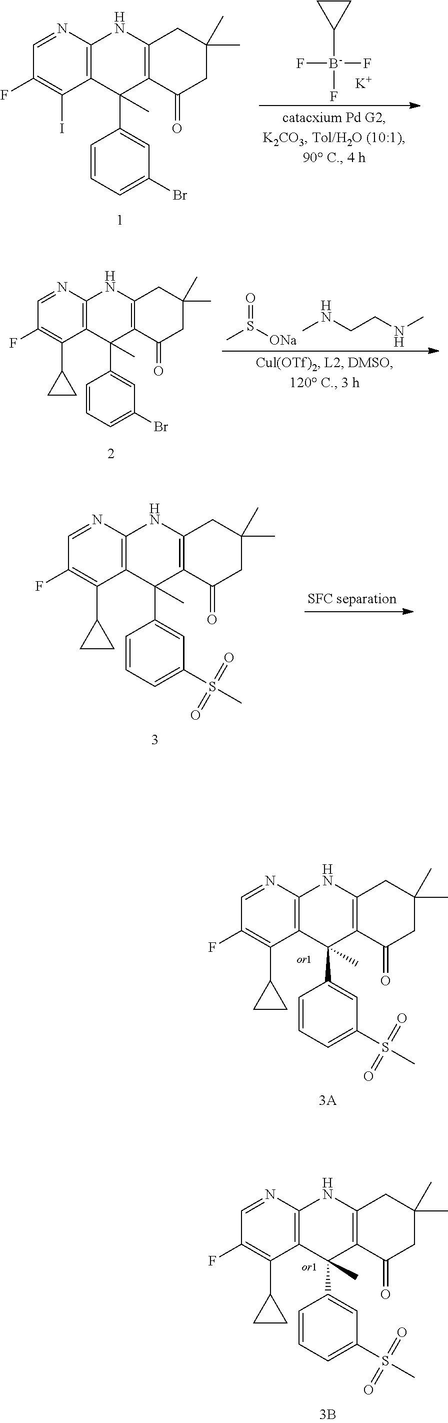

### Step 1: 5-fluoro-3-iodopyridin-2-amine (2)

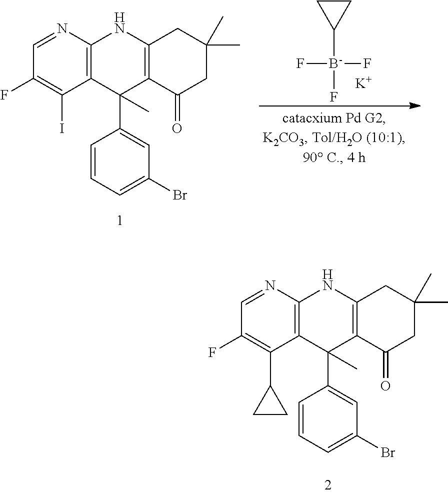 [SMILES: CC1(C)CC(=O)C2=C(C1)Nc1ncc(F)c(I)c1C2(C)c1cccc(Br)c1 ; F[B-](F)(F)C1CC1 ; [K+] ; CC1(C)CC(=O)C2=C(C1)Nc1ncc(F)c(C3CC3)c1C2(C)c1cccc(Br)c1]

To a solution of compound 1 (275 mg, 0.50 mmol, 1.00 eq) K₂CO₃ (210 mg, 1.52 mmol, 3.00 eq) and potassium cyclopropyl(trifluoro)borate (83 mg, 0.55 mmol, 1.10 eq) in toluene (3 mL) and H₂O (0.3 mL) was added cataCXium Pd G2 (43 mg, 0.05 mmol, 0.10 eq) in one portion to the reaction mixture. The reaction mixture was stirred at 90° C. for 4 h. On completion, the reaction mixture was diluted with EtOAc (3×30 mL). The organic layer was washed with brine (2×30 mL), dried over Na₂SO₄, filtered, and concentrated in vacuo. The residue was purified by flash silica gel chromatography (ISCO®; 10 g SepaFlash® Silica Flash Column, Eluent of 0˜10% Ethyl acetate/Petroleum ether gradient @ 60 mL/min) to give compound 2 (150 mg, 65% yield) as a white solid.

LCMS: tR=0.520 min., (ES⁺) m/z (M+H)⁺=457.0.

### Step 2: (S)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (3A) & (R)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (3B)

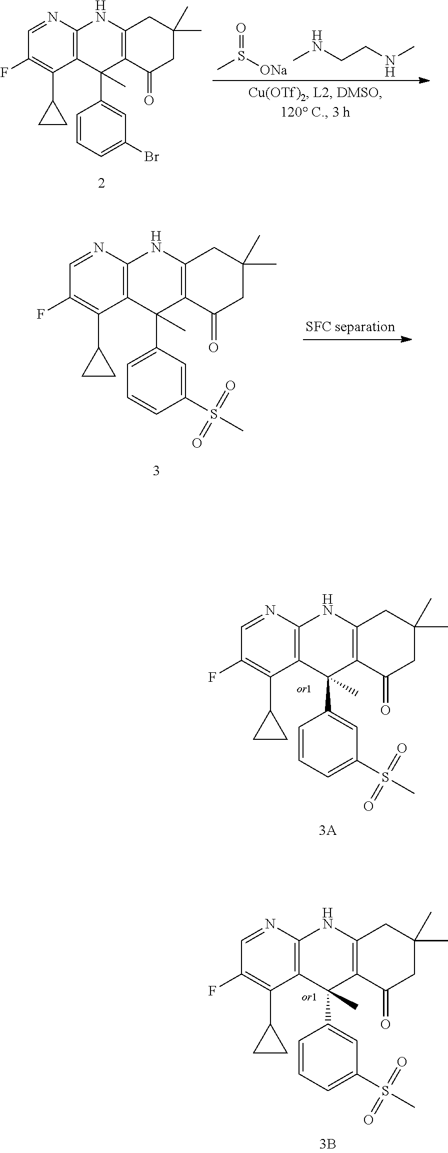

To a solution of compound 2 (150 mg, 0.02 mmol, 1.00 eq) bis(trifluoromethylsulfonyloxy) copper (118 mg, 0.02 mmol, 1.00 eq) methylsulfinyloxysodium (4.5 mg, 0.04 mmol, 2.00 eq) in DMSO (5 mL) was added N,N′-dimethylethane-1,2-diamine (4.1 mg, 0.04 mmol, 2.10 eq) dropwise to the reaction mixture. The reaction mixture was stirred at 120° C. for 3 h. On completion, the reaction mixture was diluted with EtOAc (3×20 mL). The organic layer was washed with brine (2×20 mL), dried over Na₂SO₄, filtered, and concentrated in vacuo. The residue was purified by prep-HPLC (column: Welch Xtimate C18 150×25 mm×um; mobile phase: [water(0.05% FA)-ACN]; B %: 24%-54%, 10 min) to give compound 3 (50 mg, 33% yield) as a white solid.

LCMS: tR=0.447 min., (ES⁺) m/z (M+H)⁺=445.3.

The product 50 mg was separated by SFC ((Method-B\AD-3-IPA (DEA)-5-40-3 mL-35T.l cm) Column: Column: Chiralpak AD-3 50×4.6 mm I.D., 3 um, Mobile phase: Phase A for CO₂, and Phase B for IPA (0.05% DEA); Gradient elution: B in A from 5% to 40%, Flow rate: 3 mL/min; Detector: PDA; Column Temp: 35 C; Back Pressure: 100 Bar) to give:

Peak 1: (S)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (5A). (3.8 mg, 8% yield).

LCMS: tR=0.436 min., (ES⁺) m/z (M+H)⁺=455.0.

¹H NMR (400 MHz, DMSO-d₆) d=9.85 (s, 1H), 7.94 (d, J=3.6 Hz, 1H), 7.87 (br s, 1H), 7.74-7.66 (m, 1H), 7.66-7.58 (m, 1H), 7.52-7.44 (m, 1H), 3.10 (s, 3H), 2.41 (d, J=1.2 Hz, 2H), 2.16 (s, 3H), 2.01-1.90 (m, 2H), 1.90-1.77 (m, 1H), 1.36 (br d, J=5.6 Hz, 1H), 1.15-1.07 (m, 1H), 0.97 (s, 3H), 0.87 (s, 3H), 0.69-0.58 (m, 1H), 0.03 (br dd, J=4.4.5.6 Hz, 1H).

Peak 2: (R)-4-cyclopropyl-3-fluoro-5,8,8-trimethyl-5-(3-(methylsulfonyl)phenyl)-7,8,9,10-tetrahydrobenzo[b][1,8]naphthyridin-6(5H)-one (5B). (5.6 mg, 11% yield).

LCMS: tR=0.436 min., (ES⁺) m/z (M+H)⁺=455.0.

¹H NMR (400 MHz, DMSO-d) d=9.84 (s, 1H), 7.94 (d, J=3.6 Hz, 1H), 7.88 (br s, 1H), 7.70 (br d, J=6.0 Hz, 1H), 7.63 (d, J=8.4 Hz, 1H), 7.51-7.43 (m, 1H), 3.10 (s, 3H), 2.42 (br d, J=1.6 Hz, 2H), 2.16 (s, 3H), 1.99-1.93 (m, 1H), 1.88-1.80 (m, 1H), 1.40-1.32 (m, 1H), 1.15-1.08 (m, 1H), 0.97 (s, 3H), 0.87 (s, 3H), 0.83 (br d, J=3.6 Hz, 1H), 0.67-0.59 (m, 1H), 0.07-0.00 (m, 1H).

### Example 1.35. (5S)-4-cyclopropyl-3-fluoro-5,8,8-trimethyl-5-phenyl-9,10-dihydro-7H-benzo[b][1,8]naphthyridin-6-one (3A)

### (5R)-4-cyclopropyl-3-fluoro-5,8,8-trimethyl-5-phenyl-9,10-dihydro-7H-benzo[b][1,8]naphthyridin-6-one (3B)

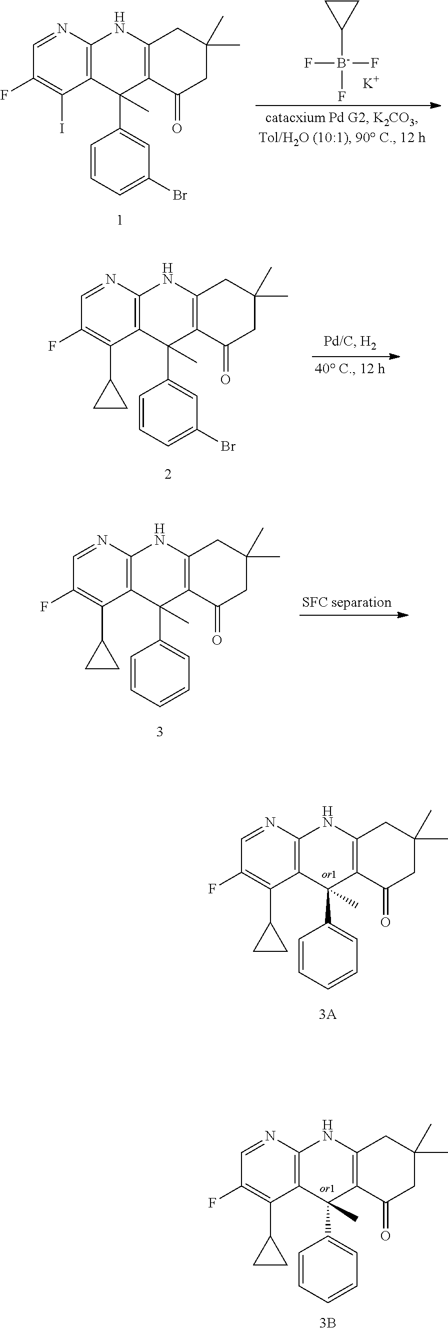

### Step 1: 5-(3-bromophenyl)-4-cyclopropyl-3-fluoro-5,8,8-trimethyl-9,10-dihydro-7H-benzo[b][1,8]naphthyridin-6-one (2)

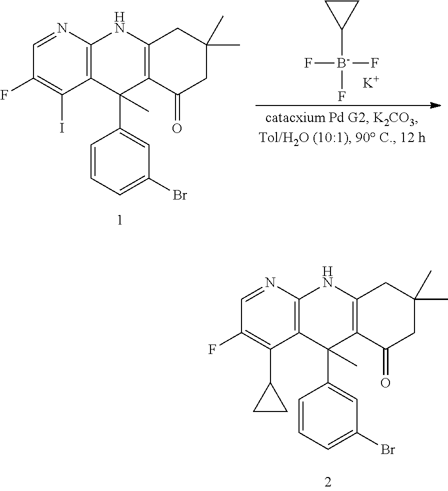 [SMILES: CC1(C)CC(=O)C2=C(C1)Nc1ncc(F)c(I)c1C2(C)c1cccc(Br)c1 ; F[B-](F)(F)C1CC1 ; [K+] ; CC1(C)CC(=O)C2=C(C1)Nc1ncc(F)c(C3CC3)c1C2(C)c1cccc(Br)c1]

To a solution of compound 1 (300 mg, 0.55 mmol, 1.0) eq), catacxium Pd G2 (37 mg, 0.0554 mmol, 0.100 eq), K₂CO₃ (230 mg, 1.66 mmol, 3.00 eq) and Potassium cyclopropyltrifluoroborate (82 mg, 0.554 mmol, 1.00 eq) in tol. (3 mL) and H₂O (0.3 mL). The reaction mixture was stirred at 90° C. for 12 h under N₂. On completion, the reaction mixture was concentrated under vacuum. The yellow residue was purified by column chromatography (SiO2, PE:EA=1:0-3:1) to give compound 2 (200 mg, crude) as a yellow solid.

LCMS: tR=1.794 min., (ES⁺) m/z (M+H)⁺=455.9.

### Step 2: (5S)-4-cyclopropyl-3-fluoro-5,8,8-trimethyl-5-phenyl-9,10-dihydro-7H-benzo[b][1,8] naphthyridin-6-one (3A)

### (5R)-4-cyclopropyl-3-fluoro-5,8,8-trimethyl-5-phenyl-9,10-dihydro-7H-benzo[b][1,8] naphthyridin-6-one (3B)

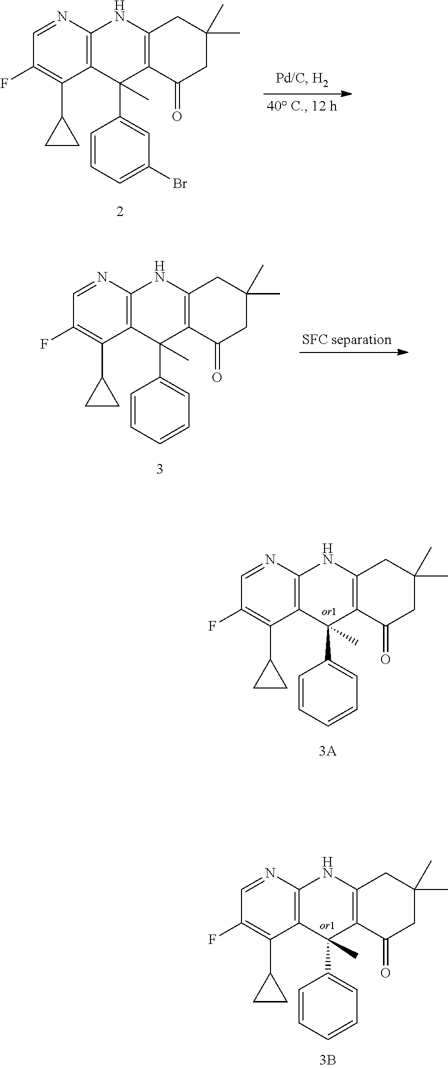 [SMILES: CC1(C)CC(=O)C2=C(C1)Nc1ncc(F)c(C3CC3)c1C2(C)c1cccc(Br)c1 ; CC1(C)CC(=O)C2=C(C1)Nc1ncc(F)c(C3CC3)c1C2(C)c1ccccc1 ; CC1(C)CC(=O)C2=C(C1)Nc1ncc(F)c(C3CC3)c1[C@]2(C)c1ccccc1 ; CC1(C)CC(=O)C2=C(C1)Nc1ncc(F)c(C3CC3)c1[C@@]2(C)c1ccccc1]

To a solution of Pd/C (2000 mg, 10% w/t, 0.1 eq) in MeOH (3 mL) was added compound 2 (150 mg, 0.33 mmol, 1.00 eq) was stirred at 40° C. for 12 h under H2 at 15 Psi. On completion, the mixture was filtered and concentrated under vacuum. The residue was purified by prep-HPLC (column: Welch Xtimate C18 150×25 mm×5 um; mobile phase: [water (NH₄HCO₃)-ACN]; B %: 42%-54%, 10 min) to compound 3 (20 mg, 97% purity, 16% yield) as a yellow solid.

The product was separated by SFC (Column: Chiralpak AS-3 50×4.6 mm I.D., 3 μm; Mobile phase: Phase A for CO₂, and Phase B for IPA (0.05% DEA); Gradient elution: IPA (0.05% DEA) in CO₂ from 5% to 40%; Flow rate: 3 mL/min Wavelength: 220 nm) to give:

Peak 1: (5S)-4-cyclopropyl-3-fluoro-5,8,8-trimethyl-5-phenyl-9,10-dihydro-7H-benzo[b][1,8] naphthyridin-6-one (3A).

(5.5 mg, 28% yield).

LCMS: tR=0.527 (ES⁺) m/z (M+H)⁺=377.2.

¹H NMR (400 MHz, DMSO-d₆) δ=9.69 (s, 1H), 7.88 (d, J=3.6 Hz, 1H), 7.34 (d, J=7.6 Hz, 2H), 7.18 (t, J=7.6 Hz, 2H), 7.07-6.99 (m, 1H), 2.38 (d, J=5.2 Hz, 2H), 2.14 (s, 3H), 1.98-1.92 (m, 1H), 1.85-1.77 (m, 11H), 1.58-1.50 (m, 1H), 1.05-1.00 (m, 11H), 0.97 (s, 3H), 0.86 (s, 3H), 0.81 (d, J=5.6 Hz, 2H), 0.12-0.03 (m, 1H).

Peak 2: (5R)-4-cyclopropyl-3-fluoro-5,8,8-trimethyl-5-phenyl-9,10-dihydro-7H-benzo[b][1,8] naphthyridin-6-one (3B).

(5.9 mg, 30% yield

LCMS: tR=0.524 (ES⁺) m/z (M+H)⁺=377.2.

¹H NMR (400 MHz, DMSO-d₆) δ=9.69 (s, 1H), 7.88 (d, J=3.6 Hz, 1H), 7.34 (d, J=7.6 Hz, 2H), 7.18 (t, J=7.6 Hz, 2H), 7.07-7.01 (m, 1H), 2.38 (d, J=4.8 Hz, 2H), 2.14 (s, 3H), 1.95 (d, J=15.6 Hz, 1H), 1.84-1.77 (m, 1H), 1.57-1.51 (m, 1H), 1.03 (d, J=5.2 Hz, 1H), 0.97 (s, 3H), 0.86 (s, 3H), 0.81 (d, J=5.6 Hz, 2H), 0.11-0.04 (m, 1H).

### Example 1.36. (5S)-3-fluoro-5,8,8-trimethyl-6-oxo-5-phenyl-9,10-dihydro-7H-benzo[b][1,8]naphthyridine-4-carbonitrile (2A)

### (5R)-3-fluoro-5,8,8-trimethyl-6-oxo-5-phenyl-9,10-dihydro-7H-benzo[b][1,8]naphthyridine-4-carbonitrile (2B)

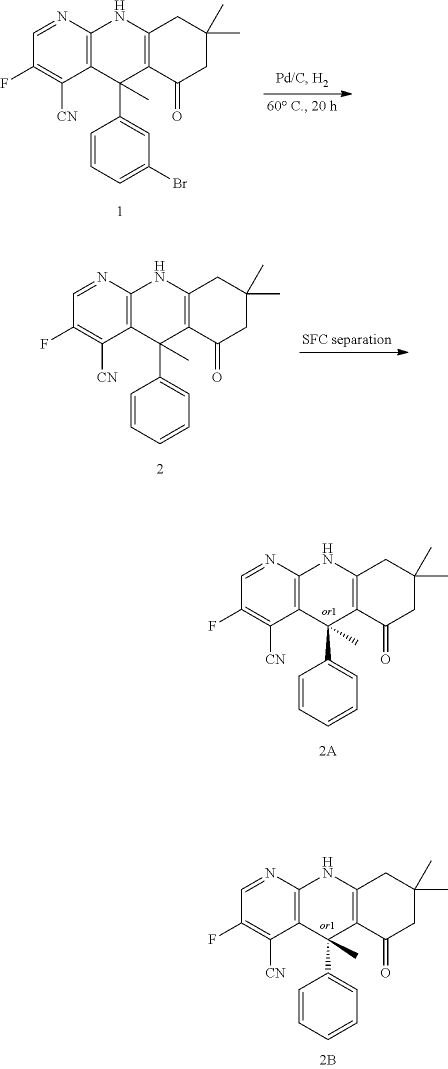 [SMILES: CC1(C)CC(=O)C2=C(C1)Nc1ncc(F)c(C#N)c1C2(C)c1cccc(Br)c1 ; CC1(C)CC(=O)C2=C(C1)Nc1ncc(F)c(C#N)c1C2(C)c1ccccc1 ; CC1(C)CC(=O)C2=C(C1)Nc1ncc(F)c(C#N)c1[C@]2(C)c1ccccc1 ; CC1(C)CC(=O)C2=C(C1)Nc1ncc(F)c(C#N)c1[C@@]2(C)c1ccccc1]

To a solution of Pd/C (150 mg, 10% w/t, 0.1 eq) in MeOH (3 mL) was added compound (140 mg, 0.32 mmol, 1.00 eq). The reaction mixture was stirred at 60° C. for 20 h under H₂ at 15 Psi. On completion, the mixture was filtered and concentrated under vacuum. The residue was purified by prep-HPLC (column: Welch Xtimate C18 150×25 mm×10 um; mobile phase: [water (NH₄HCO₃)-ACN]; B %: 30%-60%, 10 min) to give compound 3 (40 mg, 99% purity, 35% yield) as a yellow solid.

The product was separated by SFC (Column: Chiralpak IG-3 50×4.6 mm I.D., 3 um, Mobile phase: Phase A for CO₂, and Phase B for IPA (0.05% DEA); Gradient elution: B in A from 5% to 40%; Flow rate: 3 mL/min Wavelength: 220 nm) to give:

Peak 1: 5S)-3-fluoro-5,8,8-trimethyl-6-oxo-5-phenyl-9,10-dihydro-7H-benzo[b][1,8]naphthyridine-4-carbonitrile (2A). (6.8 mg, 17% yield).

LCMS: tR=0.443 (ES⁺) m/z (M+H)⁺=362.3.

¹H NMR (400 MHz, DMSO-d₆) δ=10.24 (s, 1H), 8.39 (s, 1H), 7.35 (d, J=7.6 Hz, 2H), 7.23 (t, J=7.6 Hz, 2H), 7.13-7.07 (m, 1H), 2.44 (s, 2H), 2.14 (s, 3H), 2.02-1.98 (m, 1H), 1.91-1.85 (m, 1H), 1.23 (s, 3H), 0.99 (s, 3H), 0.91 (s, 3H).

Peak 2: (5R)-3-fluoro-5,8,8-trimethyl-6-oxo-5-phenyl-9,10-dihydro-7H-benzo[b][1,8]naphthyridine-4-carbonitrile (2B). (7.8 mg, 19% yield).

LCMS: tR=0.444 (ES⁺) m/z (M+H)⁺=362.3.

¹H NMR (400 MHz, DMSO-d₆) δ=10.24 (s, 1H), 8.39 (s, 1H), 7.35 (d, J=7.6 Hz, 2H), 7.23 (t, J=7.6 Hz, 2H), 7.13-7.07 (m, 1H), 2.44 (s, 2H), 2.14 (s, 3H), 2.02-1.98 (m, 1H), 1.91-1.85 (m, 1H), 0.99 (s, 3H), 0.91 (s, 3H).

### Example 1.37. (S)-5,8,8-trimethyl-6-oxo-5-phenyl-3-(trifluoromethyl)-5,6,7,8,9,10-hexahydrobenzo[b][1,8]naphthyridine-4-carbonitrile (9A)

### (R)-5,8,8-trimethyl-6-oxo-5-phenyl-3-(trifluoromethyl)-5,6,7,8,9,10-hexahydrobenzo[b][1,8]naphthyridine-4-carbonitrile (9B)

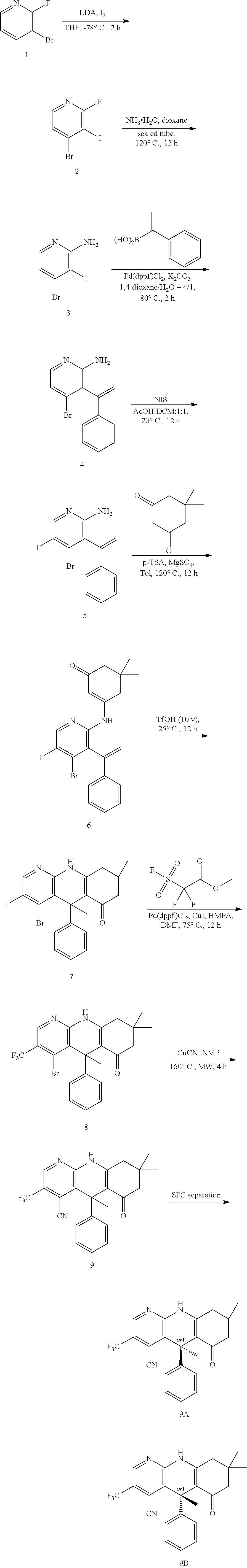

### Step 1: 4-bromo-2-fluoro-3-iodopyridine (2)

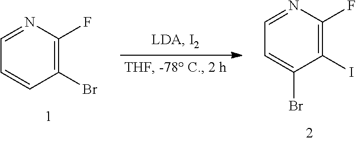 [SMILES: Fc1ncccc1Br ; Fc1nccc(Br)c1I]

To a solution of compound 1 (50.00 g, 284 mmol 1.00 eq) in THF (503.88 mL) was added LDA (2 M in THF, 156.26 mL, 313 mmol, 1.10 eq) dropwise stirred under N₂ at −70° C. for 2 h, then the mixture was stirred 12 (108.24 g, 426 mmol, 1.50 eq) under N₂ at −70° C. for 1 h. On completion, the reaction mixture was quenched by addition of saturated NH₄Cl (600 mL) at 20° C. and extracted with EtOAc (3×600 mL), washed with saturated NaCl (900 mL), dried over Na₂SO₄, filtered, and concentrated under reduced pressure to give a residue. The residue was purified by flash silica gel chromatography (ISCO®; 330 g SepaFlash® Silica Flash Column, Eluent of 4˜10% Ethyl acetate/Petroleum ether gradient (J 80 mL/min) to give compound 2 (70.00 g, 82% yield) as a yellow solid.

¹H NMR (400 MHz, DMSO-d6) δ=8.08 (d, J=5.2 Hz, 1H), 7.69 (dd, J=0.8, 5.2 Hz, 1H).

### Step 2: 4-bromo-3-iodopyridin-2-amine (3)

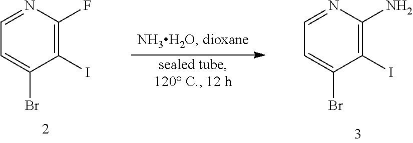 [SMILES: Fc1nccc(Br)c1I ; "sealed tube, 120° C., 12 h" ; Nc1nccc(Br)c1I]

To a solution of compound 2 (60.00 g, 199 mmol, 1.00 eq) in 1,4-dioxane (735 mL) was added NH₃·H₂O (138.02 mL, 994 mmol, 5.00 eq) stirred at 120° C. for 12 h in a pressure kier. On completion, the reaction was diluted with water (1000 mL), extracted with EtOAc (3×1000 mL), washed with saturated NaCl (1500 mL), dried over Na₂SO₄, filtered, and concentrated under reduced pressure to give a residue. The residue was purified by flash silica gel chromatography (ISCO®; 330 g SepaFlash® Silica Flash Column, Eluent of 4˜8% Ethyl acetate/Petroleum ether gradient (80 mL/min) to give compound 3 (49.50 g, 83% yield) as a yellow solid.

¹H NMR (400 MHz, DMSO-d6) δ=7.76 (d, J=5.2 Hz, 1H), 6.83 (d, J=5.2 Hz, 1H), 6.41 (br s, 2H).

### Step 3: 4-bromo-3-(1-phenylvinyl)pyridin-2-amine (4)

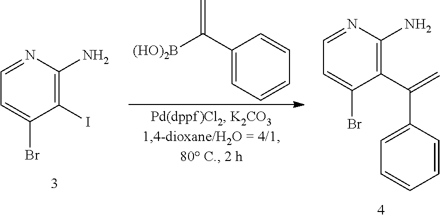

To a solution of compound 3 (31.00 g, 104 mmol, 1.00 eq) and 1-phenylvinylboronic acid (18.42 g, 124 mmol, 1.20 eq) in 1,4-dioxane (280 mL) and H₂O (70 mL) was added K₂CO₃ (43.00 g, 311 mmol, 3.00 eq) and Pd(dppf)Cl₂ CH₂Cl₂ (8.47 g, 10.4 mmol, 0.100 eq) in one portion stirred under

N₂ at 80° C. for 2 h. On completion, the reaction was diluted with water (400 mL), extracted with EtOAc (3×400 mL), washed with saturated NaCl (600 mL), dried over Na₂SO₄, filtered, and concentrated under reduced pressure to give a residue. The residue was purified by flash silica gel chromatography (ISCO®; 120 g SepaFlash® Silica Flash Column, Eluent of 18˜25% Ethyl acetate/Petroleum ether gradient @ 100 mL/min) to give compound 4 (24.00 g, 84% yield) as a yellow solid.

¹H NMR (400 MHz, DMSO-d6) δ=7.81 (d, J=5.2 Hz, 1H), 7.36-7.28 (m, 5H), 6.87 (d, J=5.2 Hz, 1H), 6.11 (s, 1H), 5.83 (s, 2H), 5.30 (s, 1H).

### Step 4: 4-bromo-5-iodo-3-(1-phenylvinyl)pyridin-2-amine (5)

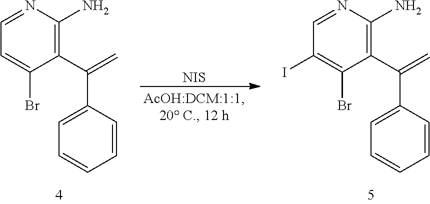

To a solution of compound 4 (24.00 g, 87.2 mmol, 1.00 eq) in AcOH (40 mL) and DCM (40 mL) was stirred at 20° C. for 12 h. On completion, the reaction was basified with NaHCO₃ to pH=9, the residue was diluted with water (100 mL), extracted with EtOAc (3×100 mL), washed with saturated Na₂S₂O₃ (150 mL), washed with saturated NaCl (150 mL), dried over Na₂SO₄, filtered, and concentrated under reduced pressure to give a residue. The residue was purified by flash silica gel chromatography (ISCO®; 330 g SepaFlash® Silica Flash Column, Eluent of 22˜35% Ethyl acetate/Petroleum ether gradient @ 100 mL/min) to give compound 5 (31.30 g, 89% yield) as a yellow solid.

¹H NMR (400 MHz, DMSO-d6) δ=8.26 (s, 1H), 7.35-7.33 (m, 2H), 7.32 (s, 3H), 6.07 (s, 1H), 6.01 (br s, 2H), 5.29 (s, 1H).

### Step 5: 3-((4-bromo-5-iodo-3-(1-phenylvinyl)pyridin-2-yl)amino)-5,5-dimethylcyclohex-2-en-1-one (6)

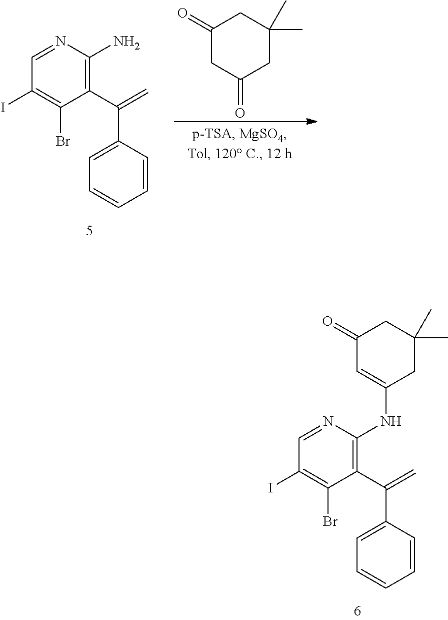

To a solution of compound 5 (31.30 g, 78.0 mmol, 1.00 eq) and 5,5-dimethylcyclohexane-1,3-dione (12.03 g, 85.8 mmol, 1.10 eq) in toluene (313 mL) was added p-TSA·H₂O (1.48 g, 7.80 mmol, 0.100 eq) and MgSO₄ (37.44 g, 312 mmol, 4.00 eq) in one portion stirred at 120° C. for 12 h. On completion, the reaction was diluted with water (300 mL), extracted with EtOAc (3×300 mL), washed with saturated NaCl (500 mL), dried over Na₂SO₄, filtered, and concentrated under reduced pressure to give a residue. The residue was purified by flash silica gel chromatography (ISCO®0; 330 g SepaFlash® Silica Flash Column, Eluent of 45˜58% Ethyl acetate/Petroleum ether gradient @ 100 mL/min) to give compound 6 (18.30 g, 45% yield) as a yellow solid.

¹H NMR (400 MHz, DMSO-d6) δ=8.73 (s, 1H), 7.80 (s, 1H), 7.36-7.29 (m, 4H), 7.28-7.25 (m, 2H), 6.33 (s, 1H), 6.20 (s, 1H), 5.44 (s, 1H), 2.29-2.20 (m, 2H), 2.00 (s, 2H), 0.89 (s, 3H), 0.84 (s, 3H).

### Step 6: 4-bromo-3-iodo-5,8,8-trimethyl-5-phenyl-5,8,9,10-tetrahydrobenzo[b][1,8]naphthyridin-6(7H)-one (7)

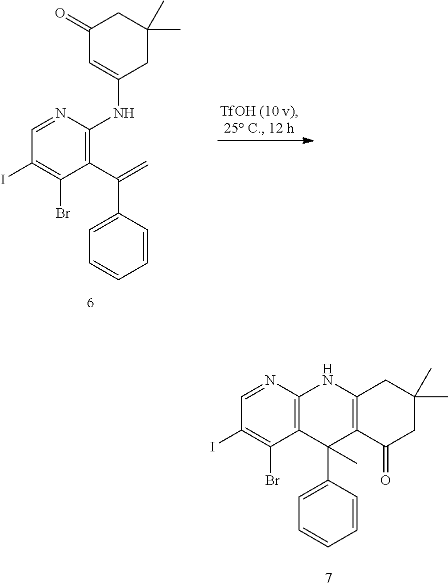 [SMILES: C=C(c1ccccc1)c1c(NC2=CC(=O)CC(C)(C)C2)ncc(I)c1Br ; CC1(C)CC(=O)C2=C(C1)Nc1ncc(I)c(Br)c1C2(C)c1ccccc1]

To a solution of compound 6 (12.00 g, 22.9 mmol, 1.00 eq) in TfOH (120 mL) was stirred at 25° C. for 12 h. On completion, the reaction was basified with NaOH to pH=9, the residue was diluted with water (200 mL), extracted with EtOAc (3×200 mL), washed with saturated NaCl (300 mL), dried over Na₂SO₄, filtered, and concentrated under reduced pressure to give a residue. The residue was purified by flash silica gel chromatography (ISCO®; 120 g SepaFlash® Silica Flash Column, Eluent of 11˜15% Ethyl acetate/Petroleum ether gradient @ 100 mL/min) to give compound 7 (9.30 g, 78% yield) as a yellow solid.

¹H NMR (400 MHz, DMSO-d6) δ=10.01 (s, 1H), 8.44 (s, 1H), 7.30 (br d, J=8.4 Hz, 2H), 7.14 (t, J=7.6 Hz, 2H), 7.05-6.99 (m, 1H), 2.40 (br d, J=3.2 Hz, 2H), 2.14 (s, 3H), 1.99-1.92 (m, 1H), 1.86-1.79 (m, 1H), 0.96 (s, 3H), 0.86 (s, 3H).

### Step 7: 4-bromo-5,8,8-trimethyl-5-phenyl-3-(trifluoromethyl)-5,8,9,10-tetrahydrobenzo[b][1,8]naphthyridin-6(7H)-one (8)

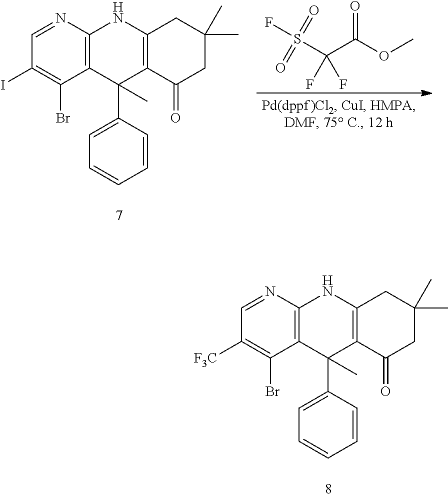

To a solution of compound 7 (3.00 g, 5.73 mmol, 1.00 eq) in DMF (15 ML) was added HMPA (3.08 g, 17.2 mmol, 3.00 eq), Pd(dppf)Cl₂CH₂Cl₂ (464 mg, 0.57 mmol, 0.100 eq) and CuI (2.18 g, 11.5 mmol, 2.00 eq) in one portion stirred under N₂ at 75° C. for 1.5 h. Then a solution of methyl 2,2-difluoro-2-fluorosulfonyl-acetate (5.51 g, 28.7 mmol, 5.00 eq) in DMF (15 mL) was added dropwise to the mixture, the mixture was stirred under N₂ at 75° C. for 16 h. On completion, the reaction was diluted with water (50 mL), extracted with EtOAc (3×50 mL), washed with NH₃·H₂O (90 mL), washed with saturated NaCl (90 mL), dried over Na₂SO₄, filtered, and concentrated under reduced pressure to give a residue. The crude product was purified by reversed phase to give compound 8 (1.80 g, 67% yield) as a yellow solid.

### Step 8: (S)-5,8,8-trimethyl-6-oxo-5-phenyl-3-(trifluoromethyl)-5,6,7,8,9,10-hexahydrobenzo [b] [1,8]naphthyridine-4-carbonitrile (9A)

### (R)-5,8,8-trimethyl-6-oxo-5-phenyl-3-(trifluoromethyl)-5,6,7,8,9,10-hexahydrobenzo[b][1,8] naphthyridine-4-carbonitrile (9B)

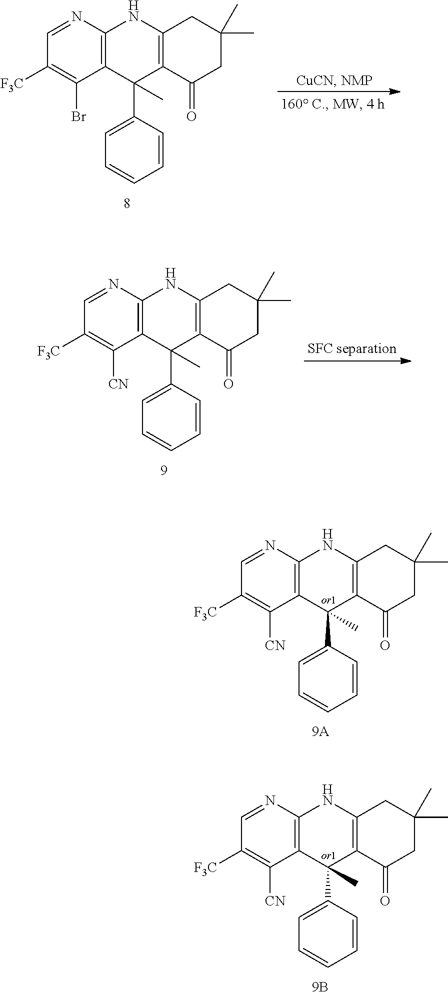

To a solution of compound 8 (200 mg, 0.430 mmol, 1.00 eq) in NMP (2 mL) was added CuCN (191 mg, 2.15 mmol, 5.00 eq) stirred at 160° C. for 4 h. On completion, the reaction was diluted with water (10 mL), extracted with EtOAc (3×10 mL), washed with NH₃·H₂O (15 mL) washed with saturated NaCl (15 mL), dried over Na₂SO₄, filtered, and concentrated under reduced pressure to give a residue. The residue was purified by prep-HPLC (Column: Phenomenex luna C18 150×25 mm×10 um, water (0.1% FA)-ACN, 55-65% ACN, 10 min) to give 100 mg product.

100 mg compound 9 was separated by SFC (Whelk-IPA (DEA)-5-40-3 mL-35T.l cm; Column: Chiralpak Whelk 50×4.6 mm I.D., 3.5 um, Mobile phase: Phase A for CO₂, and Phase B for IPA (0.05% DEA); Gradient elution: B in A from 5% to 40% Flow rate: 3 mL/min; Detector: PDA; Column Temp: 35 C; Back Pressure: 100 Bar) to give:

Peak 1: (S)-5,8,8-trimethyl-6-oxo-5-phenyl-3-(trifluoromethyl)-5,6,7,8,9,10-hexahydrobenzo [b][1,8]naphthyridine-4-carbonitrile (9A) (35 mg, 20% yield).

LCMS: tR=0.466 min., (ES⁺) m/z (M+H)⁺=412.6.

¹H NMR (400 MHz, DMSO-d6) δ=10.68 (s, 1H), 8.61 (s, 1H), 7.35 (br d, J=7.6 Hz, 2H), 7.23 (t, J=7.6 Hz, 2H), 7.16-7.06 (m, 1H), 2.47 (br s, 2H), 2.18 (s, 3H), 2.05-1.98 (m, 1H), 1.94-1.87 (m, 1H), 0.99 (s, 3H), 0.91 (s, 3H).

Peak 2: (R)-5,8,8-trimethyl-6-oxo-5-phenyl-3-(trifluoromethyl)-5,6,7,8,9,10-hexahydrobenzo[b][1,8]naphthyridine-4-carbonitrile (9B) (34 mg, 18% yield).

LCMS: tR=0.470 min., (ES⁺) m/z (M+H)⁺=412.7.

¹H NMR (400 MHz, DMSO-d6) δ=10.68 (s, 1H), 8.61 (s, 1H), 7.35 (br d, J=7.6 Hz, 2H), 7.23 (t, J=7.6 Hz, 2H), 7.14-7.08 (m, 1H), 2.47 (s, 2H), 2.18 (s, 3H), 2.04-1.98 (m, 1H), 1.93-1.87 (m, 1H), 0.99 (s, 3H), 0.91 (s, 3H).

### Example 1.38. (S)-5-(3-(3-methoxy-5-neopentylpyridin-4-yl)phenyl)-5,8,8-trimethyl-7,8,9,10-tetrahydrobenzo[b][1,8]naphthyridin-6(5H)-one (9)

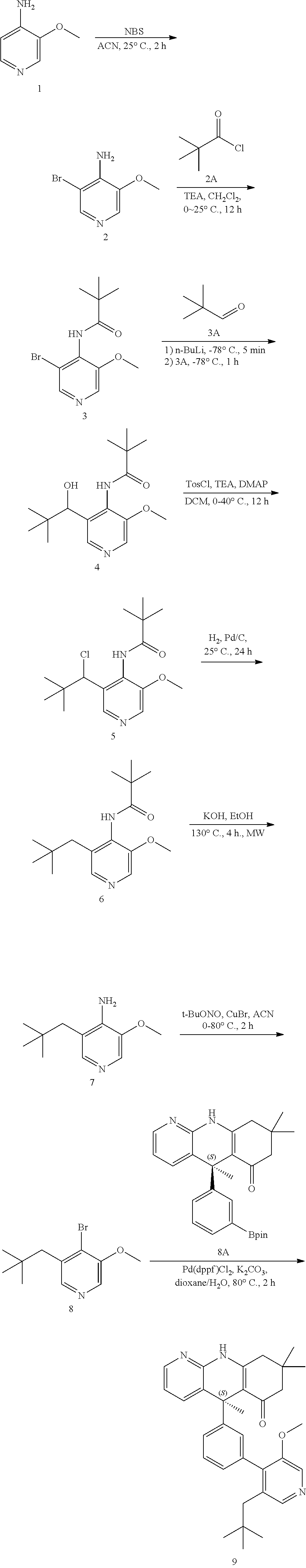

### Step 1: 3-bromo-5-methoxypyridin-4-amine. (2)

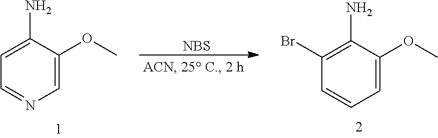

To a solution of 3-methoxypyridin-4-amine (4.00 g, 32.2 mmol, 1 eq) in ACN (40 mL) was added NBS (6.31 g, 35.4 mmol, 1.1 eq) portion wise with the temperature kept at 25° C. to get a red solution. The reaction mixture was stirred at 25° C. for 2 h. On completion, the reaction mixture was concentrated directly to remove ACN. The reaction mixture was quenched with H₂O (20 mL), and then extracted with EtOAc (30 mL×2). The combined organic layers were washed with brine (30 mL×3), dried over Na₂SO₄, filtered, and concentrated under reduced pressure to give a residue. The yellow residue was purified by column chromatography (SiO₂, Petroleum ether/Ethyl acetate=10:1-1:1) to give compound 2 (6.00 g, 92% yield) as a red solid.

¹H NMR (400 MHz, DMSO-d₆) δ=11.01 (br s, 2H), 7.91 (s, 1H), 7.82 (s, 1H), 3.79 (s, 3H).

### Step 2: N-(3-bromo-5-methoxypyridin-4-yl)pivalamide (3)

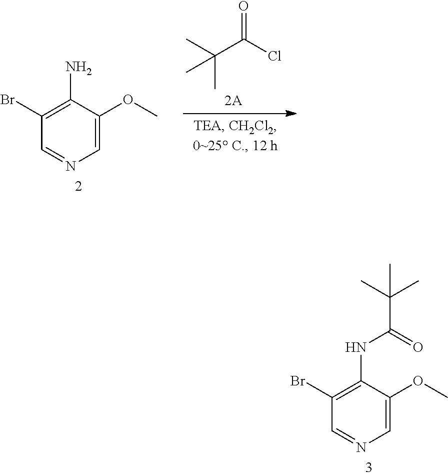

To a solution of compound 2 (6.00 g, 29.6 mmol, 1.00 eq) and TEA (8.97 g, 88.7 mmol, 3.00 eq) in CH₂Cl₂ (60 mL) was added 2,2-dimethylpropanoyl chloride (5.34 g, 44.3 mmol, 1.50 eq) portion wise with the temperature kept at 0° C. under N₂ to get a red solution. The reaction mixture was stirred at 25° C. for 12 h. On completion, the reaction mixture was quenched with H₂O (50 mL), and then extracted with DCM (30 mL×3). The combined organic layers were washed with brine (30 mL×3), dried over Na₂SO₄, filtered, and concentrated under reduced pressure to give a residue. The residue was purified by column chromatography (SiO₂, Petroleum ether/Ethyl acetate=5/1 to 3/1) to give compound 3 (4.50 g, 53% yield) as a yellow solid.

LCMS: tR=0.298 min., (ES⁺) m/z (M+H)⁺=289.0.

### Step 3: N-(3-(1-hydroxy-2,2-dimethylpropyl)-5-methoxypyridin-4-yl)pivalamide (4)

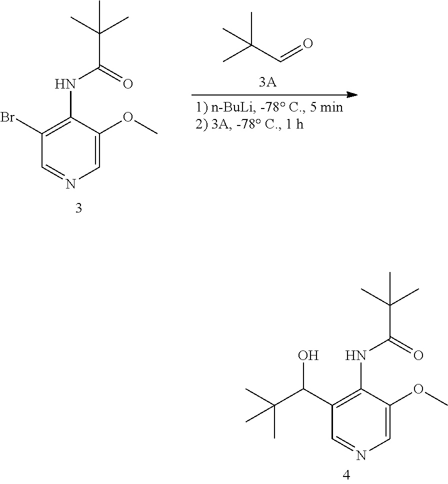

To a solution of compound 3 (4.50 g, 15.7 mmol, 1 eq) in THF (45 mL) was added n-BuLi (2.5 M, 13.7 mL, 34.5 mmol, 2.2 eq) dropwise with the temperature kept at −78° C. under N₂ to get a yellow solution. The reaction mixture was stirred at −78° C. for 5 min. Then 2,2-dimethylpropanal (13.5 g, 157 mmol, 10 eq) was added to the mixture kept at −78° C. and stirred at −78° C. for 1 h. On completion, the reaction mixture was quenched with saturated NH₄Cl (40 mL) kept at −78° C., and then extracted with EtOAc (50 mL×3). The combined organic layers were washed with brine (50 mL×3), dried over Na₂SO₄, filtered, and concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient (a 80 mL/min) to give compound 4 (1.30 g, 28% yield) as a yellow solid.

¹H NMR (400 MHz, DMSO-d₆) δ=8.86 (s, 1H), 8.21 (d, J=2.0 Hz, 2H), 5.30 (br d, J=3.2 Hz, 1H), 4.45 (br s, 1H), 3.81 (s, 3H), 1.21 (s, 10H), 0.81 (s, 9H), 2.13-1.28 (m, 1H).

### Step 4: N-(3-(1-chloro-2,2-dimethylpropyl)-5-methoxypyridin-4-yl)pivalamide (5)

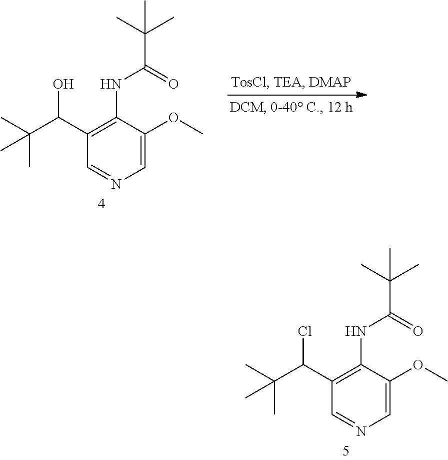 [SMILES: COc1cncc(C(O)C(C)(C)C)c1NC(=O)C(C)(C)C ; COc1cncc(C(Cl)C(C)(C)C)c1NC(=O)C(C)(C)C]

To a solution of compound 4 (1.30 g, 4.42 mmol, 1.00 eq) and DMAP (54 mg, 0.442 mmol, 0.1 eq), TEA (1.34 g, 13.2 mmol, 3 eq) in DCM (13 mL) was added 4-methylbenzenesulfonyl chloride (1.68 g, 8.83 mmol, 2 eq) portion wise with the temperature kept at 25° C. under N₂ to get a yellow solution. The reaction mixture was stirred at 40° C. for 12 h. On completion, the reaction mixture was quenched with NH₄Cl (15 mL), and then extracted with DCM (15 mL×3). The combined organic layers were washed with brine (15 mL×3), dried over Na₂SO₄, filtered, and concentrated under reduced pressure to give a residue. The yellow residue was purified by column chromatography (SiO₂, Ethyl acetate/Petroleum ether gradient=5:1-3:1) to give compound 5 (700 mg, 51.0% yield) as a yellow solid.

LCMS: tR=0.355 min., (ES⁺) m/z (M+H)⁺=313.1.

### Step 5: N-(3-methoxy-5-neopentylpyridin-4-yl)pivalamide (6)

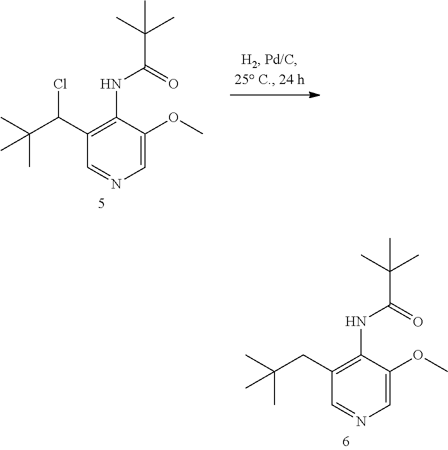 [SMILES: COc1cncc(C(Cl)C(C)(C)C)c1NC(=O)C(C)(C)C ; COc1cncc(CC(C)(C)C)c1NC(=O)C(C)(C)C]

To a solution of Pd/C (350 mg, 10% purity, 1 eq) in MeOH (7 mL) was added compound 5 (700 mg, 2.24 mmol, 1.00 eq) portion wise with the temperature kept at 25° C. under H₂ to get a yellow solution. The reaction mixture was stirred at 40° C. for 4 h. On completion, the reaction mixture was filtered and concentrated under reduced pressure to give compound 6 (500 mg, 80% yield) as a yellow solid.

LCMS: tR=0.294 min., (ES⁺) m/z (M+H)⁺=279.4.

### Step 6: 3-methoxy-5-neopentylpyridin-4-amine (7)

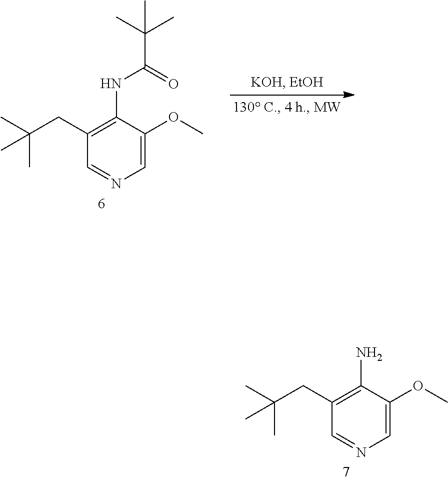 [SMILES: COc1cncc(CC(C)(C)C)c1NC(=O)C(C)(C)C ; COc1cncc(CC(C)(C)C)c1N]

To a solution of compound 6 (500 mg, 1.80 mmol, 1 eq) in EtOH (5 mL) was added KOH (2.5 M, 2.16 mL, 5.39 mmol, 3.00 eq) portion wise with the temperature kept at 25° C. get a white solution. The reaction mixture was stirred at 130° C. for 4 h under MW reaction. On completion, the reaction mixture was concentrated directly to remove EtOH, then it was dissolved with CH₂Cl₂/MeOH (10 mL×3) and separated by filtration, the filtrate was spirally dried to give compound 7 (290 mg, 83% yield) as a white solid.

¹H NMR (400 MHz, DMSO-d₆) δ=8.90-7.63 (m, 2H), 3.95 (s, 2H), 2.75 (s, 3H), 0.93 (s, 9H).

### Step 8: 4-bromo-3-methoxy-5-neopentylpyridine (8)

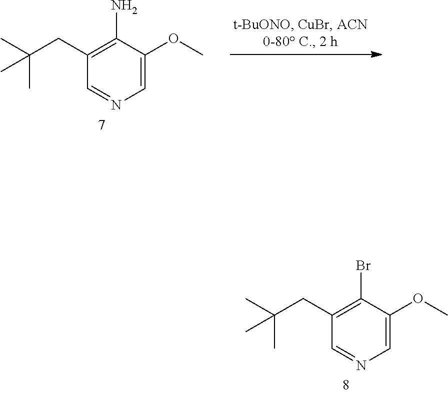

To a solution of compound 7 (240 mg, 1.24 mmol, 1.00 eq) and CuBr (209 mg, 1.48 mmol, 1.20 eq) in ACN (2.5 mL) was added t-BuONO (318 mg, 3.09 mmol, 2.50 eq) portion wise with the temperature kept at 0° C. to get a yellow solution. The reaction mixture was stirred at 80° C. for 2 h. On completion, the reaction mixture was quenched with NaHCO₃ (15 mL), and then extracted with EtOAc (15 mL×3). The combined organic layers were washed with brine (15 mL×3), dried over Na₂SO₄, filtered, and concentrated under reduced pressure to give a residue. The residue was purified by column chromatography (SiO2, Ethyl acetate/Petroleum ether gradient=0:1-3:1) to give compound 8 (210 mg, 66% yield) as a yellow solid.

LCMS: tR=0.376 min., (ES⁺) m/z (M+H)⁺=259.8.

### Step 9: (S)-5-(3-(3-methoxy-5-neopentylpyridin-4-yl)phenyl)-5,8,8-trimethyl-5,8,9,10-tetrahydrobenzo[b][1,8]naphthyridin-6(7H)-one (9)

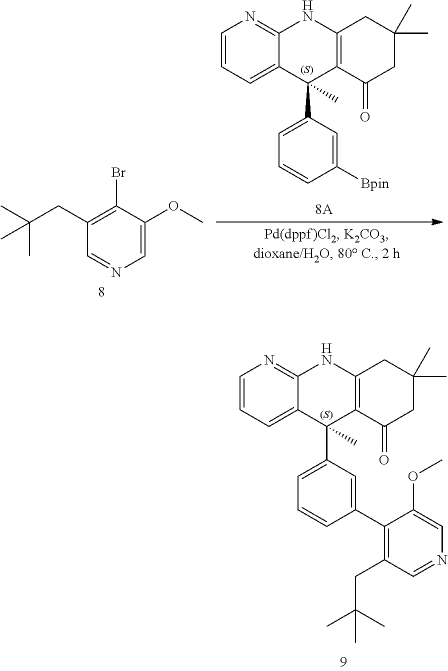

To a solution of 8A (100 mg, 225 μmol, 1.00 eq) and compound 8 (58 mg, 0.225 mmol, 1.00 eq) in dioxane (1 mL) and H₂O (1 mL) was added Pd(dppf)Cl₂ (16 mg, 0.0225 mmol, 0.1 eq) and K₂CO₃ (93 mg, 0.675 mmol, 3 eq) portion wise with the temperature kept at 25° C. under N₂ to get a yellow solution. The reaction mixture was stirred at 80° C. for 2 h. On completion, the reaction mixture was quenched with saturated NH₄Cl (10 mL), and then extracted with EtOAc (10 mL×3). The combined organic layers were washed with brine (10 mL×3), dried over Na₂SO₄, filtered, and concentrated under reduced pressure to give a residue. The crude product was purified by re-MPLC (0.1% FA, 5%-95% ACN/H₂O) to give (S)-5-(3-(3-methoxy-5-neopentylpyridin-4-yl)phenyl)-5,8,8-trimethyl-5,8,9,10-tetrahydrobenzo[b][1,8]naphthyridin-6(7H)-one (20.7 mg, 18% yield) as a white solid.

LCMS: tR=0.383 min., (ES⁺) m/z (M+H)⁺=496.4.

¹H NMR (400 MHz, DMSO-d) δ=9.80-9.67 (m, 1H), 8.21 (br d, J=11.6 Hz, 1H), 8.09 (br d, J=9.2 Hz, 1H), 7.94 (br s, 1H), 7.48-7.36 (m, 1H), 7.35-7.26 (m, 1H), 7.11 (br t, J=8.4 Hz, 1H), 7.03 (br d, J=14.8 Hz, 1H), 6.96-6.84 (m, 1H), 6.79 (br s, 1H), 3.73-3.60 (m, 4H), 2.35-2.25 (m, 1H), 2.04-1.96 (m, 2H), 1.91 (br d, J=15.2 Hz, 3H), 1.23 (s, 3H), 1.06-0.92 (m, 7H), 0.57 (s, 4H), 0.45 (s, 5H).

### Example 1.39. (S)-5-(3-bromophenyl)-5,8,8-trimethyl-7,8,9,10-tetrahydrobenzo[b][1,8]naphthyridin-6(5H)-one. (6A)

### (S)-5,8,8-trimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one. (6B)

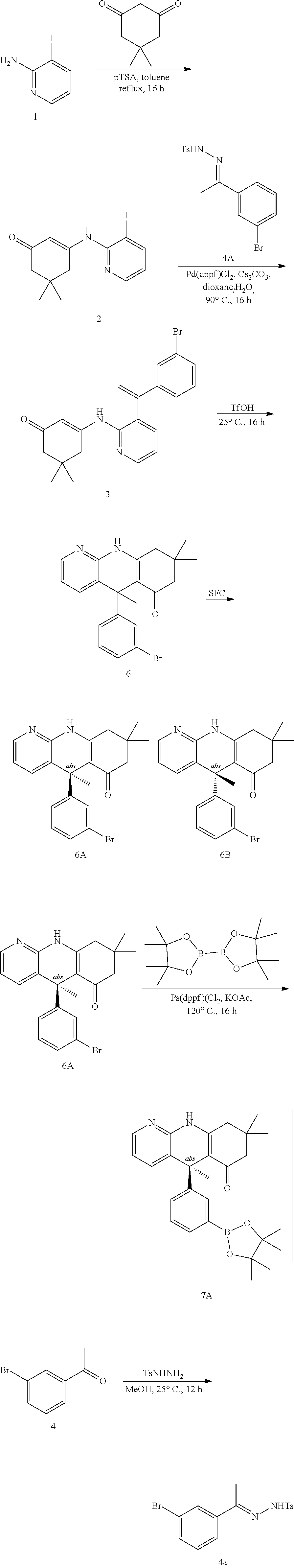

### Step 1: 3-((3-iodopyridin-2-yl)amino)-5,5-dimethylcyclohex-2-enone (2)

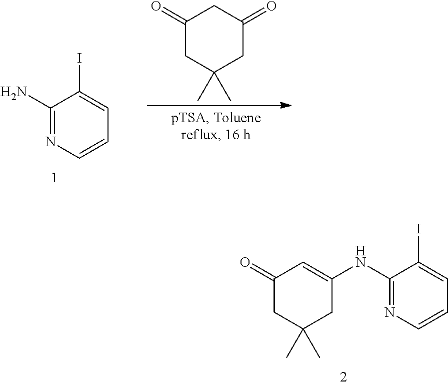

To a solution 3-iodopyridin-2-amine (5.00 g, 22.7 mmol, 1.00 eq) and 5,5-dimethylcyclohexane-1,3-dione (3.18 g, 22.7 mmol, 1.00 eq) in Toluene (30 mL) was added pTSA (432 mg, 2.27 mmol, 0.100 eq) portion wise with the temperature kept at 25° C. The reaction mixture was stirred at 120° C. for 16 h. On completion, the mixture quenched by 200 ml H₂O and basified to pH=7 with Na₂CO₃. The mixture was filtered, and the filtrate was extracted with EtOAc (3×50 mL), washed with saturated NaCl (50 mL), dried over Na₂SO₄, filtered, and concentrated. The residue was purified by flash silica gel chromatography (ISCO®; 10 g SepaFlash® Silica Flash Column, Eluent of 10% Ethyl acetate/Petroleum ether gradient @ 60 mL/min) to give compound 2 (5.20 g, 62% yield) as a yellow solid.

LCMS: tR=0.793 min., (ES⁺) m/z (M+H)⁺=343.0.

### Step 2: 3-((3-iodopyridin-2-yl)amino)-5,5-dimethylcyclohex-2-enone (3)

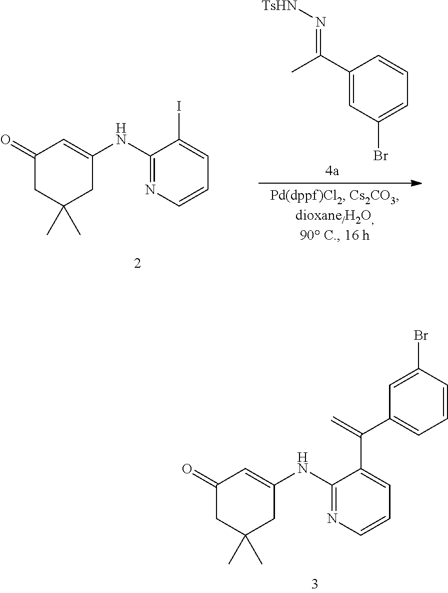

To a solution compound 2 (18.00 g, 52.6 mmol, 1.00 eq) and compound 4a (19.32 g, 52.6 mmol, 1.00 eq) in dioxane (50 mL) and H₂O (50 mL) was added to Pd(dppf)Cl₂ (3.85 g, 5.26 mmol, 0.1 eq) and Cs₂CO₃ (51.42 g, 157.81 mmol, 3 eq). The mixture was stirred at 90° C. for 16 h. The reaction mixture was stirred at 120° C. for 16 h. On completion, the mixture was diluted with H₂O (20 mL) extracted with EtOAc (3×20 mL). The organic layer was washed with saturated NaCl (20 mL), dried over Na₂SO₄, filtered, and concentrated in vacuo. The residue was purified by prep-HPLC to give compound 3 (10.00 g, 43% yield) as a yellow solid.

LCMS: tR=0.430 min., (ES⁺) m/z (M+H)⁺=399.1.

### Step 3: 5-(3-bromophenyl)-5,8,8-trimethyl-7,8,9,10-tetrahydrobenzo[b][1,8]naphthyridin-6(5H)-one (6)

### (S)-5-(3-bromophenyl)-5,8,8-trimethyl-7,8,9,10-tetrahydrobenzo[b][1,8]naphthyridin-6(5H)-one (6A)

### (R)-5-(3-bromophenyl)-5,8,8-trimethyl-7,8,9,10-tetrahydrobenzo[b][1,8]naphthyridin-6(5H)-one (6B)

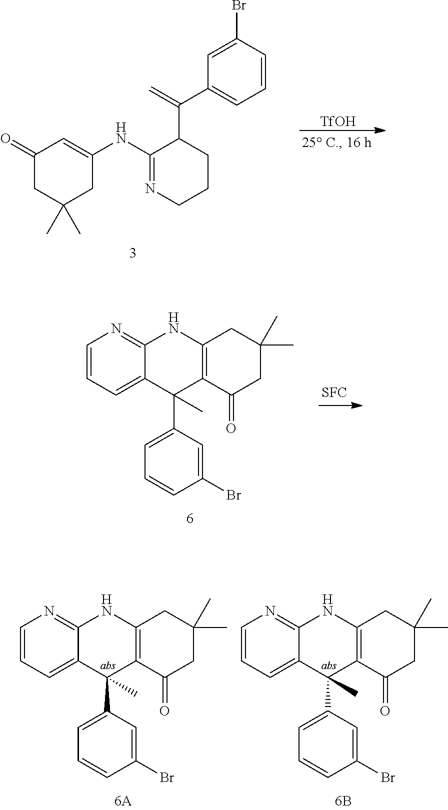

To a solution compound 3 (6.00 g, 15.1 mmol, 1.00 eq) in TfOH (20 mL) was stirred at 25° C. for 16 h. The mixture quenched by 100 ml H₂O and basified to pH=7 with Na₂CO₃. The mixture was filtered, and the filter cake was dried in vacuo. The residue was purified by flash silica gel to give compound 6 (4.8 g).

The product was separated by SFC ((S,S)Whelk-O1 50×4.6 mm I.D., 1.8 um; mobile phase: [0.05% DEA MeOH]; B %: 5%-40%, B 5.5; 180 min) to give:

Peak 1: (S)-5-(3-bromophenyl)-5,8,8-trimethyl-7,8,9,10-tetrahydrobenzo[b][1,8]naphthyridin-6(5H)-one (6A) (3.00 g, 49% yield).

LCMS: tR=0.436 min., (ES⁺) m/z (M+H)⁺=399.1.

¹H NMR (400 MHz, CD₃OD-d4) d=7.97 (dd, J=1.6, 4.8 Hz, 1H), 7.53 (t, J=2.0 Hz, 1H), 7.37 (td, J=1.2, 8.0 Hz, 1H), 7.28-7.22 (m, 1H), 7.19-7.13 (m, 1H), 7.09 (dd, J=1.6, 7.6 Hz, 1H), 6.82 (dd, J=5.2, 7.6 Hz, 1H), 2.61-2.50 (m, 2H), 2.17-2.06 (m, 2H), 1.92 (s, 3H), 1.10 (d, J=2.8 Hz, 6H).

Peak 2: (R)-5-(3-bromophenyl)-5,8,8-trimethyl-7,8,9,10-tetrahydrobenzo[b][1,8]naphthyridin-6(5H)-one (6B) (1.20 g, 20% yield).

LCMS: tR=0.465 min., (ES⁺) m/z (M+H)⁺=399.0.

¹H NMR (400 MHz, CD₃OD-d₄) d=8.01-7.92 (m, 1H), 7.53 (s, 1H), 7.37 (br d, J=7.6 Hz, 1H), 7.29-7.21 (m, 1H), 7.21-7.13 (m, 1H), 7.09 (dd, J=1.2, 7.6 Hz, 1H), 6.82 (dd, J=4.8, 7.6 Hz, 1H), 2.62-2.49 (m, 2H), 2.18-2.05 (m, 2H), 1.92 (s, 3H), 1.10 (d, J=2.4 Hz, 6H).

### Step 4: (E)-N′-(1-(3-bromophenyl)ethylidene)-4-methylbenzenesulfonohydrazide (4a)

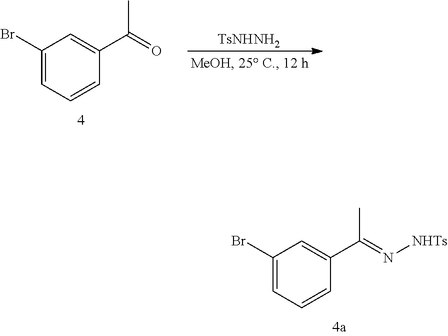

To a solution 1-(3-bromophenyl)ethanone (20.00 g, 100 mmol, 1.00 eq) and 4-methylbenzenesulfonohydrazide (18.71 g, 100 mmol, 1.00 eq) in MeOH (100 mL) was stirred at 25° C. for 12 h. On completion, the crude product was triturated with 500 mL Petroleum ether at 20° C. for 30 min to give the compound 4a (29.00 g, 73% yield) as a yellow solid.

### Step 5: (S)-5,8,8-trimethyl-5-(3-(4,4,5,5-tetramethyl-1,3,2-dioxaborolan-2-yl)phenyl)-7,8,9,10-tetrahydrobenzo[b][1,8]naphthyridin-6(5H)-one (7A)

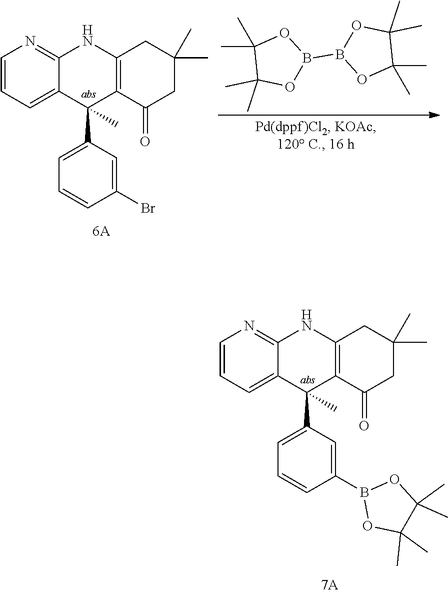

To a solution compound 6A (50 mg, 0.126 mmol, 1.00 eq) and 4,4,5,5-tetramethyl-2-(4,4,5,5-tetramethyl-1,3,2-dioxaborolan-2-yl)-1,3,2-dioxaborolane (160 mg, 0.629 mmol, 5.00 eq) in dioxane (1 mL) was added KOAc (62 mg, 0.629 mmol, 5.00 eq) and Pd(dppf)Cl₂ (18 mg, 25.2 μmol, 0.200 eq). The reaction mixture was stirred at 120° C. for 16 h. On completion, the mixture was diluted with H₂O (20 mL) extracted with EtOAc (3×20 mL). The organic layer was washed with saturated NaCl (20 mL), dried over Na₂SO₄, filtered, and concentrated in vacuo. The residue was purified by flash silica gel chromatography (ISCO®; 5 g SepaFlash® Silica Flash Column, Eluent of 45˜50% Ethyl acetate/Petroleum ether gradient (a 60 mL/min) to give the compound 7A (15 mg, 24% yield) as a yellow solid.

LCMS: tR=0.637 min., (ES⁺) m/z (M+H)⁺=445.4.

### Example 1.40. (S)-5,8,8-trimethyl-5-(3-(3-(2,2,2-trifluoroethyl)pyridin-4-yl)phenyl)-5,8,9,10-tetrahydrobenzo[b][1,8]naphthyridin-6(7H)-one (8)

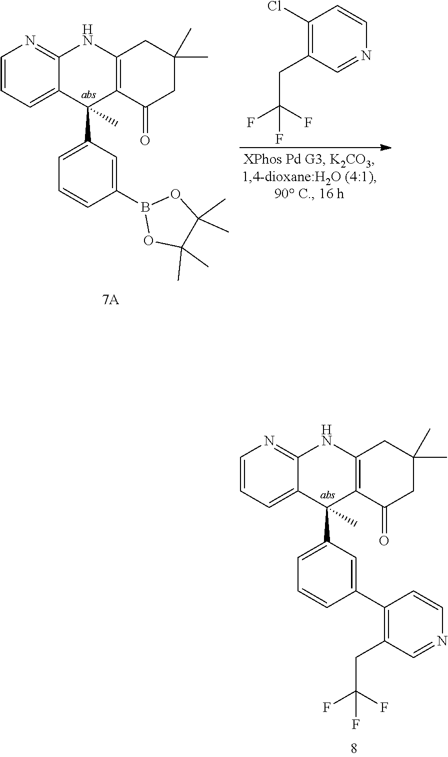 [SMILES: CC1(C)CC(=O)C2=C(C1)Nc1ncccc1[C@]2(C)c1cccc(B2OC(C)(C)C(C)(C)O2)c1 ; FC(F)(F)Cc1cnccc1Cl ; CC1(C)CC(=O)C2=C(C1)Nc1ncccc1[C@]2(C)c1cccc(-c2ccncc2CC(F)(F)F)c1]

To a solution of compound 7A (100 mg, 0.23 mmol, 1.00 eq) and 4-chloro-3-(2,2,2-trifluoroethyl)pyridine (88 mg, 0.45 mmol, 2.00 eq) in 1,4-dioxane (0.8 mL) and H₂O (0.2 mL) was added X-phos Pd G3 (19 mg, 0.02 mmol, 0.10 eq) and K₂CO₃ (93 mg, 0.67 mmol, 3.00 eq) in one portion under N₂, the mixture was stirred under N₂ at 90° C. for 16 h. On completion, the reaction was diluted with H₂O (5 mL), extracted with EtOAc (3×5 mL), washed with saturated NaCl (10 mL), dried over Na₂SO₄, filtered, and concentrated under reduced pressure to give a residue. The residue was purified by flash silica gel chromatography (ISCO®; 10 g SepaFlash® Silica Flash Column, Eluent of 0˜55% Ethyl acetate/Petroleum ether gradient @ 80 mL/min) to give 70 mg product. The 70 mg product was purified by prep-HPLC (Column: Phenomenex luna C18 150×25 mm×10 um, water (0.1% FA)-ACN, 30-60% ACN, 15 min) to give compound 8 (19 mg, 98% purity, 17% yield) as a yellow solid.

LCMS: tR=0.469 min., (ES⁺) m/z (M+H)⁺=478.2

¹H NMR (400 MHz, DMSO-d₆) δ=9.80 (s, 1H), 8.63 (s, 1H), 8.57 (d, J=5.2 Hz, 1H), 7.96 (dd, J=1.6, 4.8 Hz, 1H), 7.46-7.36 (m, 2H), 7.32-7.26 (m, 2H), 7.07 (br d, J=7.6 Hz, 2H), 6.79 (dd, J=4.8, 7.6 Hz, 1H), 3.69-3.51 (m, 2H), 2.46 (br s, 2H), 2.06-1.94 (m, 2H), 1.90 (s, 3H), 0.99 (d, J=13.2 Hz, 6H).

### Example 1.41. (S)-5-(3-(3-cyclobutylpyridin-4-yl)phenyl)-5,8,8-trimethyl-7,8,9,10-tetrahydrobenzo[b][1,8]naphthyridin-6(5H)-one (9)

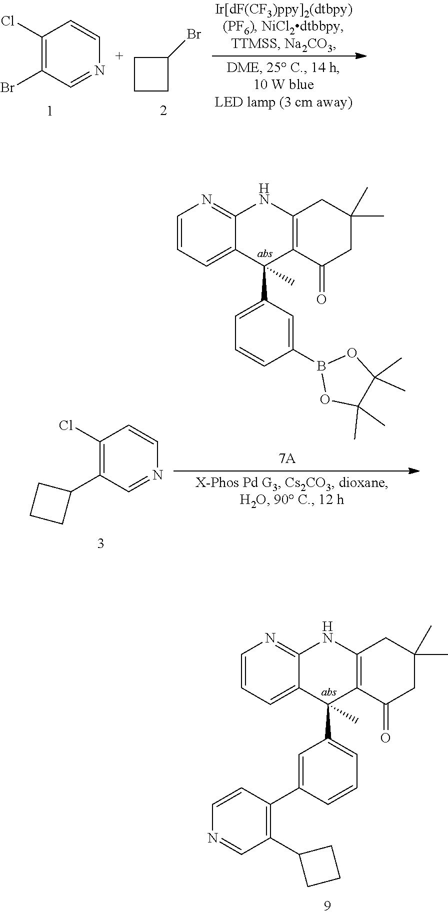

### Step 1: 4-chloro-3-cyclobutylpyridine (3)

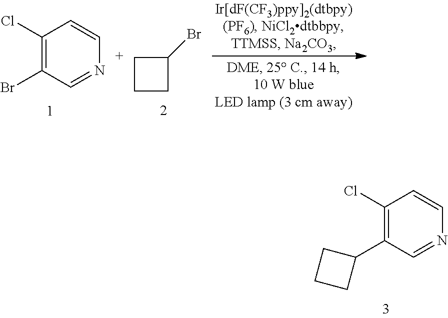

To an 15 mL vial equipped with a stir bar was add 3-bromo-4-chloropyridine (3.00 g, 15.6 mmol, 1.00 eq), bromocyclobutane (2.74 g, 20.3 mmol, 1.30 eq), Ir[dF(CF3)ppy]2(dtbpy)(PF6) (0.17 g, 0.156 mmol, 0.0100 eq), NiCl2·dtbbpy (0.093 g, 0.234 mmol, 0.0150 eq), TTMSS (3.87 g, 15.6 mmol, 1.00 eq), Na₂CO₃ (3.30 g, 31.2 mmol, 2.00 eq) in DME (200 mL). The vial was sealed under nitrogen. The reaction was stirred and irradiated with a 10 W blue LED lamp (3 cm away), with cooling water to keep the reaction temperature at 25° C. for 14 h. On completion, the reaction mixture was poured into saturated NH₄Cl aqueous solution (20 mL) and extracted with EtOAc (20 mL×3). The combined organic layer was washed with brine (50 mL), dried over Na₂SO₄, filtered, and concentrated. The residue was purified by column chromatography (SiO₂, Petroleum ether/Ethyl acetate=1/1) to give compound 3 (540 mg, 90% purity, 19% yield) as a yellow oil.

LCMS: tR=0.868 min., (ES⁺) m/z (M+H)⁺=168.1.

### Step 2: (S)-5-(3-(3-cyclobutylpyridin-4-yl)phenyl)-5,8,8-trimethyl-7,8,9,10-tetrahydrobenzo [b][1,8]naphthyridin-6(5H)-one (9)

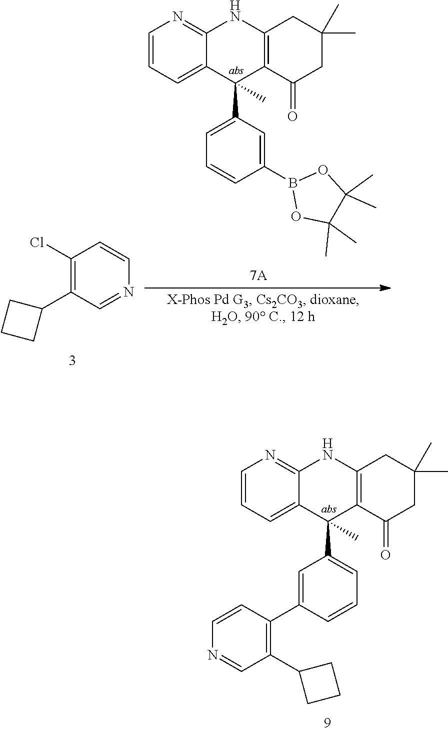 [SMILES: Clc1ccncc1C1CCC1 ; CC1(C)CC(=O)C2=C(C1)Nc1ncccc1[C@]2(C)c1cccc(B2OC(C)(C)C(C)(C)O2)c1 ; CC1(C)CC(=O)C2=C(C1)Nc1ncccc1[C@]2(C)c1cccc(-c2ccncc2C2CCC2)c1]

To a solution of compound 7A (159 mg, 0.358 mmol, 1.20 eq), compound 3 (50 mg, 0.298 mmol, 1.0) eq) and Cs₂CO₃ (291 mg, 0.895 mmol, 3.00 eq) in dioxane (0.5 mL) and H₂O (0.5 mL) was added X-phos Pd G3 (25 mg, 0.0298 mmol, 0.100 eq) in one portion under N₂ to get a yellow solution. On completion, the reaction mixture was used directly to purify. The residue was purified by prep-HPLC (column: Welch Xtimate C18 150×25 mm×5 um; mobile phase: [water (0.01% NH₃·H₂O)-ACN]; B %; 24%-54%, 10 min) to give 9 (43.7 mg, 29% yield) as a white solid.

LCMS: tR=0.369 min., (ES⁺) m/z (M+H)⁺=450.4.

¹H NMR (400 MHz, DMSO-d₆) δ=9.81 (s, 1H), 8.61 (s, 1H), 8.41 (d, J=4.8 Hz, 1H), 7.98 (dd, J=1.6, 4.8 Hz, 1H), 7.46 (d, J=8.0 Hz, 1H), 7.36 (t, J=7.6 Hz, 1H), 7.17 (s, 1H), 7.15-7.08 (m, 2H), 7.04 (d, J=7.6 Hz, 1H), 6.82 (dd, J=4.8, 7.6 Hz, 1H), 3.47-3.37 (m, 1H), 3.32 (s, 3H), 2.00 (br d, J=9.6 Hz, 2H), 1.96-1.90 (m, 4H), 1.85 (ddd, J=5.2, 7.6, 10.4 Hz, 2H), 1.71-1.62 (m, 2H), 0.98 (d, J=18.8 Hz, 6H).

### Example 1.42. (S)-5-(3-(3-(tert-butyl)pyridin-4-yl)phenyl)-5,8,8-trimethyl-7,8,9,10-tetrahydrobenzo [b][1,8]naphthyridin-6(5H)-one (10)

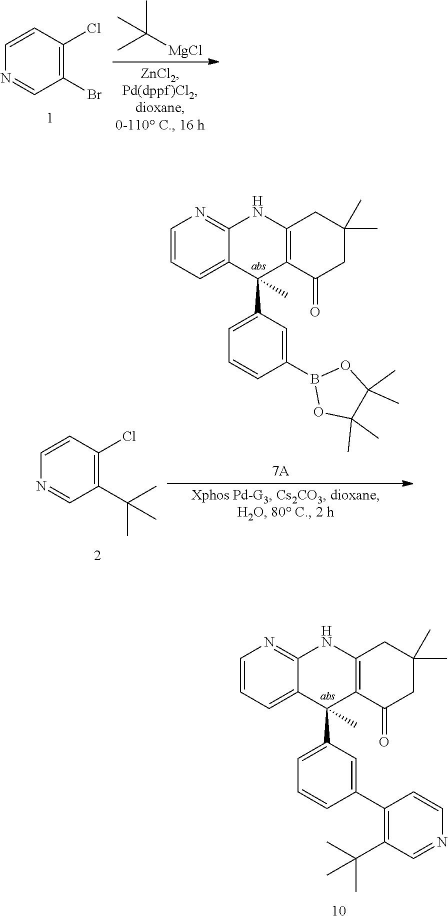

### Step 1: 3-(tert-butyl)-4-chloropyridine (2)

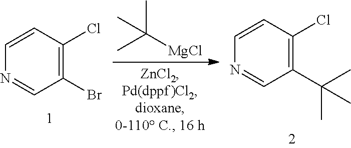

To a solution of ZnCl₂ (1 M in THF, 31.18 mL, 31.2 mmol, 2.00 eq) was added tert-butylmagnesium chloride (1.7 M in THF, 36.68 mL, 62.4 mmol, 4.00 eq) under N₂ at 0° C. The solution was diluted with dioxane (60 mL) and transferred into a suspension of 3-bromo-4-chloropyridine (3.00 g, 15.6 mmol, 1.00 eq) and Pd(dppf)Cl₂ (253 mg, 0.31 mmol, 0.02 eq) in 1,4-dioxane (30 mL). The mixture was stirred under N₂ at 110° C. for 16 h. On completion, the reaction was concentrated under reduced pressure to give a residue. The yellow residue was purified by column chromatography (SiO₂, Petroleum ether:Ethyl acetate=30:1-0:1) to give compound 2 (200 mg, 8% yield) as a yellow solid.

¹H NMR (400 MHz, DMSO-d6) d=8.48 (d, J=5.2 Hz, 1H), 7.51-7.46 (m, 1H), 7.33 (td, J=2.4, 5.2 Hz, 1H), 1.29 (d, J=1.6 Hz, 9H)

### Step 2: (S)-5-(3-(3-(tert-butyl)pyridin-4-yl)phenyl)-5,8,8-trimethyl-7,8,9,10-tetrahydrobenzo[b][1,8]naphthyridin-6(5H)-one (10)

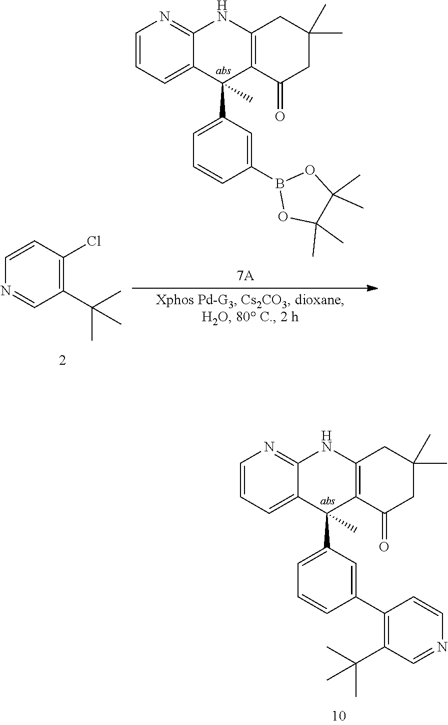 [SMILES: CC(C)(C)c1cnccc1Cl ; CC1(C)CC(=O)C2=C(C1)Nc1ncccc1[C@]2(C)c1cccc(B2OC(C)(C)C(C)(C)O2)c1 ; CC1(C)CC(=O)C2=C(C1)Nc1ncccc1[C@]2(C)c1cccc(-c2ccncc2C(C)(C)C)c1]

To a solution of compound 2 (130 mg, 0.31 mmol, 1.00 eq) and compound 7A (150 mg, 0.34 mmol, 1.10 eq) in dioxane (2 mL) and H₂O (0.5 mL) was added X-phos Pd G3 (26 mg, 0.03 mmol, 0.10 eq) and K₂CO₃ (127 mg, 0.92 mmol, 3.00 eq) in one portion under N₂, the mixture was stirred under N₂ at 80° C. for 2 h. On completion, the reaction was diluted with water (5 mL), extracted with EtOAc (3×5 mL), washed with saturated NaCl (10 mL), dried over Na₂SO₄, filtered, and concentrated under reduced pressure to give a residue. The crude product was purified by reversed phase (FA, ACN/H₂O condition) to give 50 mg product. The 50 mg product was purified by prep-HPLC (Column: Phenomenex luna C18 150×25 mm×10 um, water (FA)-ACN, 18-48% ACN, 15 min) to give compound 10 (6.5 mg, 98% purity, 5% yield) as a yellow solid.

LCMS: tR=0.436 min., (ES⁺) m/z (M+H)⁺=452.4.

¹H NMR (400 MHz, DMSO-d₆) δ=9.84 (s, 1H), 8.55 (d, J=5.2 Hz, 1H), 8.44 (br s, 1H), 7.99-7.92 (m, 1H), 7.63 (s, 1H), 7.53 (s, 1H), 7.48-7.42 (m, 2H), 7.42-7.35 (m, 2H), 7.17 (d, J=7.2 Hz, 1H), 6.79 (dd, J=4.8, 7.6 Hz, 1H), 2.55-2.51 (m, 2H), 2.10-2.05 (m, 1H), 2.01-1.92 (m, 4H), 1.35 (s, 9H), 1.04 (d, J=4.4 Hz, 6H).

### Example 1.43. (S)-5,8,8-trimethyl-5-(3-(3-(pyrrolidin-1-yl)pyridin-4-yl)phenyl)-7,8,9,10-tetrahydrobenzo [b][1,8]naphthyridin-6(5H)-one (11)

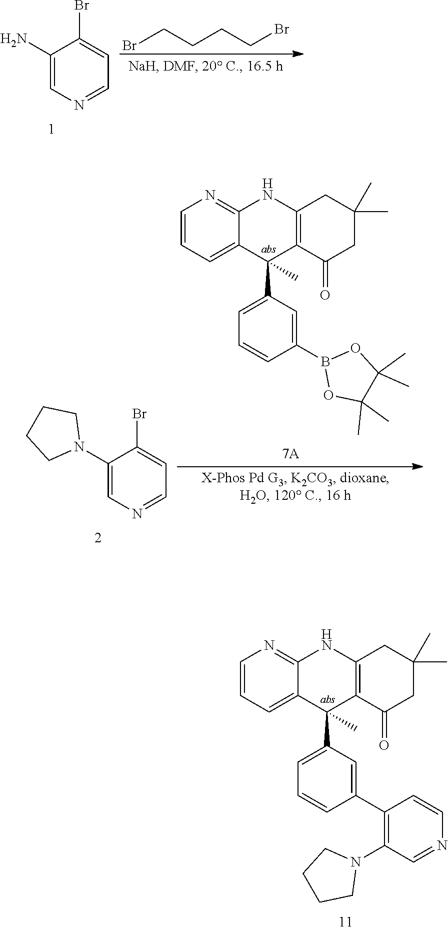

### Step 1: 4-bromo-3-(pyrrolidin-1-yl)pyridine (2)

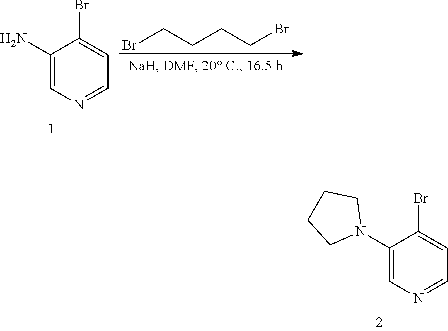

To a solution of 4-bromopyridin-3-amine (1.00 g, 5.78 mmol, 1.00 eq) in DMF (20 mL) was added NaH (462 mg, 11.6 mmol, 2.00 eq) stirred under N2 at 20° C. for 30 mins, then 1,4-dibromobutane (998 mg, 4.62 mmol, 0.800 eq) was added and stirred under N2 at 20° C. for 16 h. On completion, the reaction mixture was quenched by water (20 mL) and extracted with EtOAc (3×20 mL), washed with saturated NaCl (30 mL), dried over Na₂SO₄, filtered, and concentrated under reduced pressure to give a residue. The residue was purified by column chromatography (SiO₂, Petroleum ether/Ethyl acetate=10/1-1:1) to give compound 2 (200 mg, 14% yield) as a yellow solid.

¹H NMR (400 MHz, DMSO-d₆) δ=8.16 (s, 1H), 7.84 (d, J=5.2 Hz, 1H), 7.51 (d, J=5.2 Hz, 1H), 3.42-3.37 (m, 4H), 1.89 (td, J=3.2, 6.4 Hz, 4H).

### Step 2: (S)-5,8,8-trimethyl-5-(3-(3-(pyrrolidin-1-yl)pyridin-4-yl)phenyl)-5,8,9,10-tetrahydrobenzo[b][1,8]naphthyridin-6(7H)-one (11)

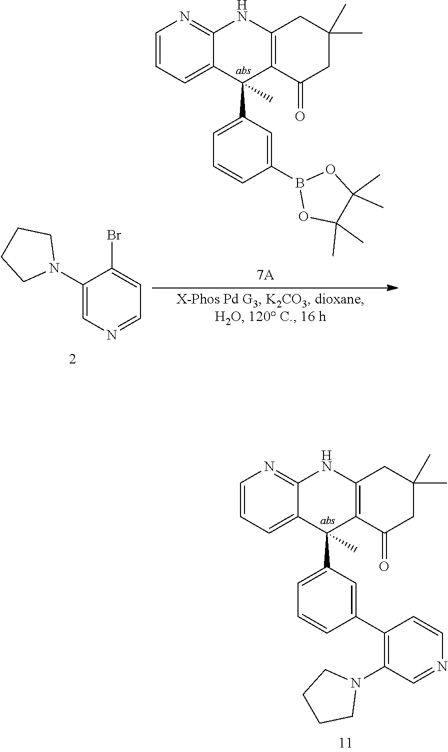 [SMILES: Brc1ccncc1N1CCCC1 ; CC1(C)CC(=O)C2=C(C1)Nc1ncccc1[C@]2(C)c1cccc(B2OC(C)(C)C(C)(C)O2)c1 ; CC1(C)CC(=O)C2=C(C1)Nc1ncccc1[C@]2(C)c1cccc(-c2ccncc2N2CCCC2)c1]

To a mixture of 7A (120 mg, 0.270 mmol, 1.00 eq), compound 2 (74 mg, 0.324 mmol, 1.20 eq) and K₂CO₃ (112 mg, 0.810 mmol, 3.00 eq) in dioxane (4 mL) and H₂O (1 mL) was added XPhos Pd G3 (23 mg, 0.0270 mmol, 0.100 eq) under N₂ and stirred at 120° C. for 16 h. The mixture quenched by 40 ml H₂O and basified to pH=7 with saturated Na₂CO₃. The mixture was filtered, and the filter cake was dried under vacuum. The residue was purified by prep-HPLC (column: Welch Xtimate C18 150×25 mm×5 um; mobile phase: [water (0.05% HCl)-ACN]; B %; 24%-54%, 10 min) and lyophilization to give 11 (17 mg, 98% purity, 14% yield) as a yellow solid.

LCMS: tR=0.363 min., (ES⁺) m/z (M+H)⁺=465.8.

¹H NMR (400 MHz, DMSO-d₆) δ=9.77 (s, 1H), 8.10 (s, 1H), 7.99-7.93 (m, 2H), 7.42-7.38 (m, 1H), 7.35-7.30 (m, 1H), 7.27 (t, J=1.6 Hz, 11H), 7.14-7.08 (m, 2H), 6.99 (d, J=4.8 Hz, 1H), 6.80 (dd, J=4.8, 8.0 Hz, 1H), 2.76-2.68 (m, 4H), 2.45 (br d, 0.1=4.0 Hz, 2H), 2.06-1.94 (m, 2H), 1.91 (s, 3H), 1.66-1.54 (m, 4H), 1.00 (s, 3H), 0.95 (s, 3H).

### Example 1.44. (S)-3-fluoro-5,8,8-trimethyl-5-(3-(3-neopentylpyridin-4-yl)phenyl)-7,8,9,10-tetrahydrobenzo[b][1,8]naphthyridin-6(5H)-one (12)

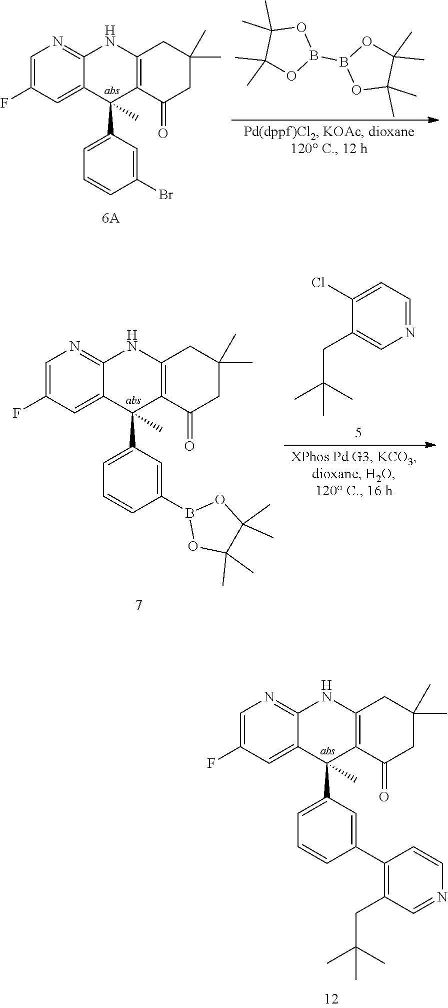

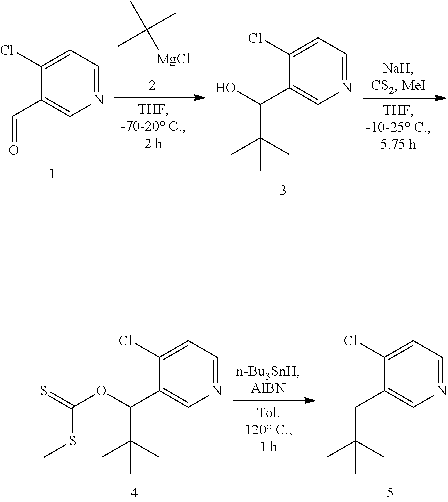

### Step 1: (S)-3-fluoro-5,8,8-trimethyl-5-(3-(4,4,5,5-tetramethyl-1,3,2-dioxaborolan-2-yl)phenyl)-7,8,9,10-tetrahydrobenzo[b][1,8]naphthyridin-6(5H)-one (7)

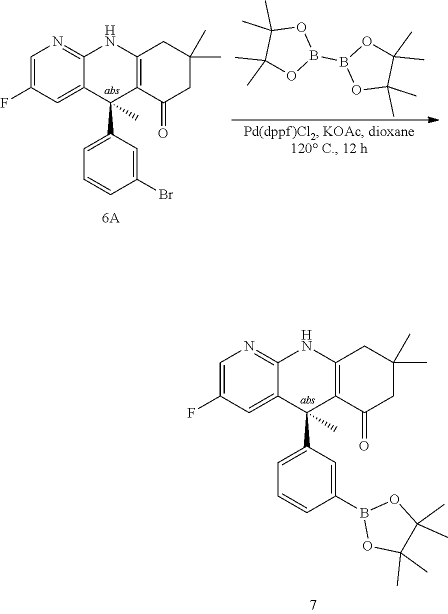

To a solution of compound 6A (200 mg, 0.482 mmol, 1.00 eq), 4,4,4′,4′,5,5,5′,5′-octamethyl-2,2′-bi(1,3,2-dioxaborolane) (611 mg, 2.41 mmol, 5.00 eq) and KOAc (236 mg, 2.41 mmol, 5.00 eq) in dioxane (4 mL) was added Pd(dppf)Cl₂ (70 mg, 96.3 μmol, 0.200 eq) under N₂ and stirred at 120° C. for 12 h. On completion, the reaction was diluted with water (20 mL), extracted with EtOAc (3×10 mL), washed with saturated NaCl (15 mL), dried over Na₂SO₄, filtered, and concentrated in vacuo to give a residue. The residue was purified by flash silica gel chromatography (ISCO®; 10 g SepaFlash® Silica Flash Column, Eluent of 0˜10% Ethyl acetate/Petroleum ether gradient @ 60 mL/min) to get compound 7 (200 mg, 92% purity, 83% yield) as a yellow solid.

### Step 2: 1-(4-chloropyridin-3-yl)-2,2-dimethylpropan-1-ol (3)

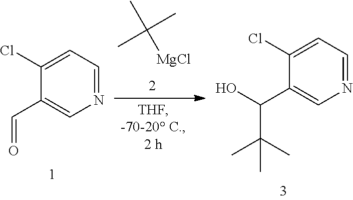

To a solution of tert-butylmagnesium chloride (292 mL, 1.00 M, 5.00 eq) in THF (40 mL) was added compound 1 (8.26 g, 70.6 mmol, 1.00 eq) portion wise with the temperature kept at −70° C. under N₂ to get a yellow solution. The reaction mixture was stirred at −20° C. for 2 h. On completion, the mixture quenched by 200 ml saturated NH₄Cl. The mixture was extracted with EtOAc (3×10 mL), washed with saturated NaCl (15 mL), dried over Na₂SO₄, filtered, and concentrated in vacuo to give a residue. The residue was purified by flash silica gel chromatography (ISCO®; 10 g SepaFlash® Silica Flash Column, Eluent of 0˜10% Ethyl acetate/Petroleum ether gradient @ 60 mL/min) to get compound 3 (4.00 g, 97% purity, 55% yield) as a yellow solid.

### Step 3: 0-(1-(4-chloropyridin-3-yl)-2,2-dimethylpropyl)S-methyl carbonodithioate (4)

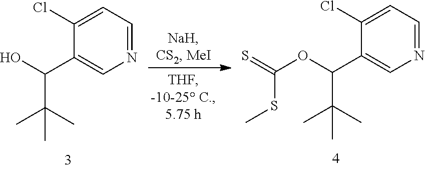 [SMILES: CC(C)(C)C(O)c1cnccc1Cl ; CSC(=S)OC(c1cnccc1Cl)C(C)(C)C]

To a solution of compound 3 (2.30 g, 7.94 mmol, 1.00 eq) in THF (20 mL) was added NaH (0.38 g, 15.9 mmol, 2.00 eq) at −10° C. under nitrogen atmosphere. The suspension was stirred at 25° C. for 0.5 h. Then CS₂ (0.73 g, 9.52 mmol, 1.20 eq) was added to the reaction mixture at −10° C. The mixture was stirred at −10° C. for 15 mins. MeI (2.25 g, 15.9 mmol, 2.00 eq) was added to the mixture dropwise. The mixture was warmed to 25° C., stirred for 5 h. On completion, the mixture quenched by 200 ml saturated NH₄Cl. The mixture was extracted with EtOAc (3×20 mL), washed with saturated NaCl (15 mL), dried over Na₂SO₄, filtered, and concentrated in vacuo to give a residue. The residue was purified by flash silica gel chromatography (ISCO®; 10 g SepaFlash® Silica Flash Column, Eluent of 0˜5% Ethyl acetate/Petroleum ether gradient @ 60 mL/min) to give compound 4 (2.70 g, crude) as a yellow solid.

### Step 4: 4-chloro-3-neopentylpyridine (5)

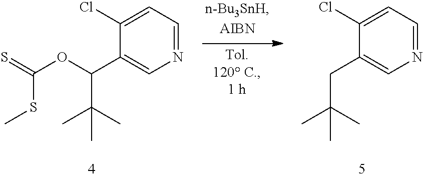 [SMILES: CSC(=S)OC(c1cnccc1Cl)C(C)(C)C ; CC(C)(C)Cc1cnccc1Cl]

To a solution of compound 4 (1.20 g, 4.14 mmol, 1.00 eq) and tributylstannane (1.205 g, 4.14 mmol, 1.00 eq) in Toluene (10 mL) was added AIBN (170 mg, 1.04 mmol, 0.25 eq) was stirred at 120° C. for 1 h. On completion, the mixture quenched by 200 ml saturated NH₄Cl. The mixture was extracted with EtOAc (3×20 mL), washed with saturated NaCl (15 mL), dried over Na₂SO₄, filtered, and concentrated in vacuo to give a residue. The residue was purified by flash silica gel chromatography (ISCO®; 10 g SepaFlash® Silica Flash Column. Eluent of 2˜4% Ethyl acetate/Petroleum ether gradient @ 60 mL/min to get compound 5 (600 mg, 90% purity, 71% yield) as a yellow solid.

### Step 5: (S)-3-fluoro-5,8,8-trimethyl-5-(3-(3-neopentylpyridin-4-yl)phenyl)-7,8,9,10-tetrahydrobenzo [b][1,8]naphthyridin-6(5H)-one (12)

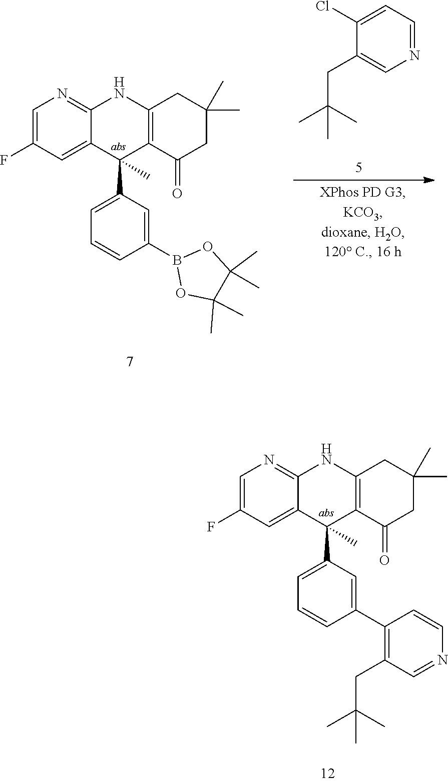 [SMILES: CC1(C)CC(=O)C2=C(C1)Nc1ncc(F)cc1[C@]2(C)c1cccc(B2OC(C)(C)C(C)(C)O2)c1 ; CC(C)(C)Cc1cnccc1Cl ; CC(C)(C)Cc1cnccc1-c1cccc([C@]2(C)C3=C(CC(C)(C)CC3=O)Nc3ncc(F)cc32)c1]

To a solution compound 7 (100 mg, 0.216 mmol, 1.00 eq), compound 5 (44 mg, 0.238 mmol, 1.10 eq) and K₂CO₃ (90 mg, 0.649 mmol, 3.00 eq) in dioxane (4 mL) and H₂O (1 mL) was added XPhos Pd G3 (18 mg, 0.0216 mmol, 0.100 eq) under N₂ and stirred at 120° C. for 16 h. The mixture quenched by 50 ml H₂O and basified to pH=7 with Na₂CO₃. The mixture was filtered, and the filter cake was dried under vacuum. The residue was purified by flash silica gel chromatography (ISCO®; 10 g SepaFlash® Silica Flash Column, Eluent of 15˜20% Ethyl acetate/Petroleum ether gradient @ 60 mL/min) to give 12 (45 mg, 98% purity, 42% yield) as a yellow solid.

LCMS: tR=0.414 min., (ES⁺) m/z (M+H)⁺=484.3.

¹H NMR (400 MHz, DMSO-d₆) δ=9.89 (s, 1H), 8.42-8.39 (m, 2H), 7.98 (d, J=2.8 Hz, 1H), 7.49-7.44 (m, 1H), 7.42-7.34 (m, 1H), 7.21 (s, 1H), 7.12 (d, J=5.2 Hz, 1H), 7.07 (d, J=7.6 Hz 1H), 7.01 (dd, J=2.8, 9.6 Hz, 1H), 2.59 (s, 2H), 2.06 (s, 2H), 1.95 (s, 2H), 1.00 (d, J=9.2 Hz, 6H), 0.49 (s, 9H).

### Example 1.45. (S)(3-bromophenyl)-3-fluoro-5,8,8-trimethyl-7,8,9,10-tetrahydrobenzo[b][1,8] naphthyridin-6(5H)-one (6A)

### (R)-5-(3-bromophenyl)-3-fluoro-5,8,8-trimethyl-7,8,9,10-tetrahydrobenzo[b][1,8] naphthyridin-6(5H)-one (6B)

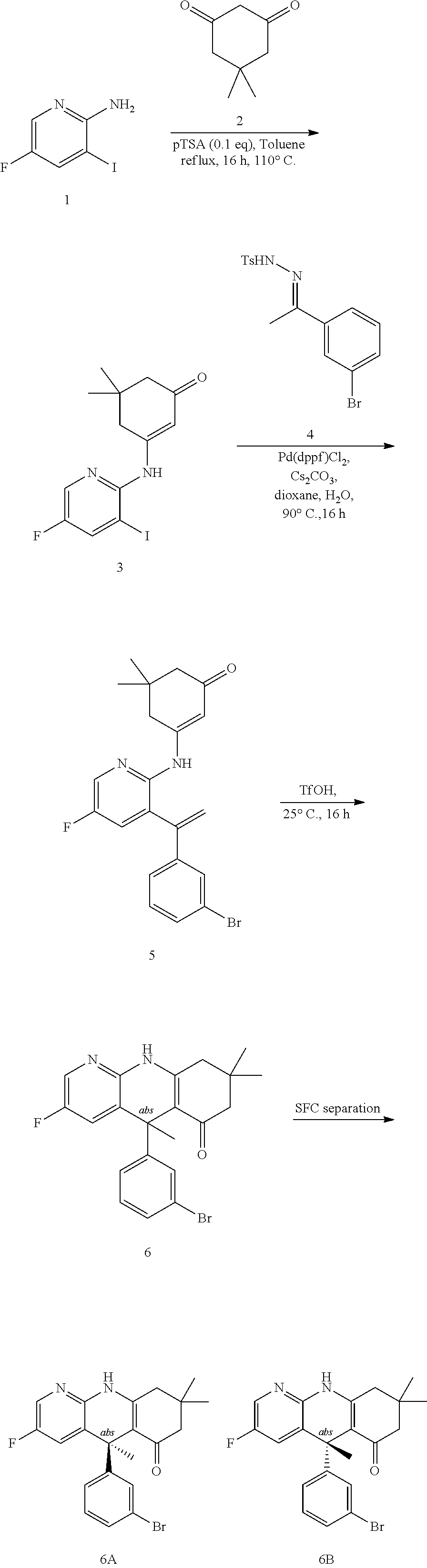

### Step 1: 3-((5-fluoro-3-iodopyridin-2-yl)amiino)-5,5-dimethylcyclohex-2-enone (3)

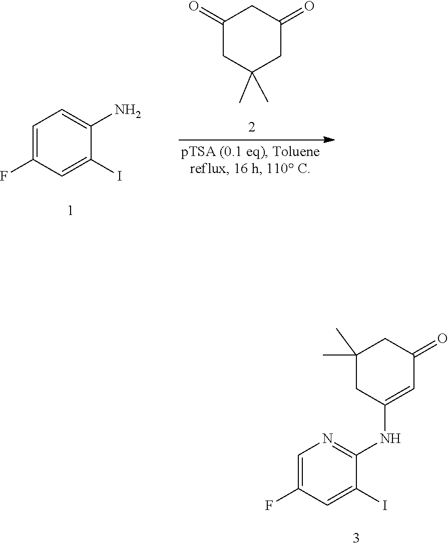

To a solution of compound 2 (14.14 g, 101 mmol, 1.20 eq), compound 1 (20.00 g, 84.0 mmol, 1.00 eq) and PTSA (1.44 g, 8.40 mmol, 0.100 eq) in Toluene (20 mL) was stirred at 110° C. by reflux for 16 h. On completion, the mixture quenched by 200 ml H₂O and basified to pH=7 with Na₂C₀₃. The mixture was filtered, and the filtrate was extracted with EtOAc (3×50 mL), washed with saturated NaCl (50 mL), dried over Na₂SO₄, filtered, and concentrated. The residue was purified by flash silica gel chromatography (ISCO®; 50 g SepaFlash® Silica Flash Column, Eluent of 18˜19%/Ethyl acetate/Petroleum @ 100 mL/min) to give compound 3 (15.50 g, 90% purity, 46% yield) as a yellow solid.

### Step 2: 3-((3-(1-(3-bromophenyl)vinyl)-5-fluoropyridin-2-yl)amino)-5,5-dimethylcyclohex-2-enone (5)

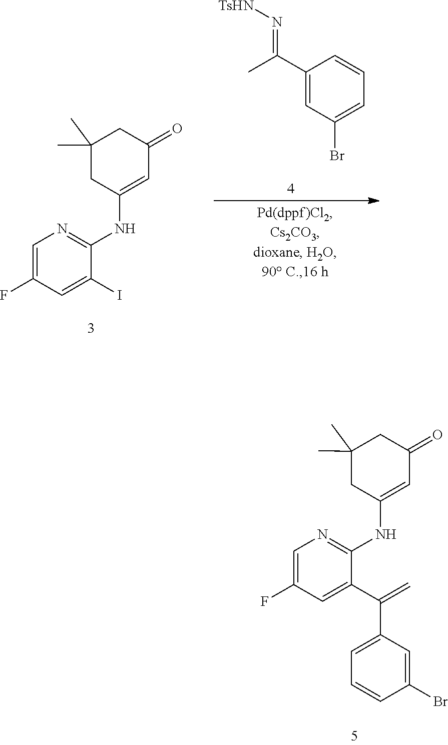

To a solution of compound 3 (10.00 g, 27.8 mmol, 1.00 eq), compound 4 (10.20 g, 27.8 mmol, 1.00 eq) and Cs₂CO₃ (27.14 g, 83.3 mmol, 3.00 eq) in dioxane (20 mL) and H₂O (20 mL) was added Pd(dppf)Cl₂ (2.03 g, 2.78 mmol, 0.100 eq) under N₂. The mixture was stirred at 90° C. for 16 h. On completion, the mixture was diluted with H₂O (20 mL) extracted with EtOAc (3×20 mL). The organic layer was washed with saturated NaCl (20 mL), dried over Na₂SO₄, filtered, and concentrated in vacuo. The residue was purified by prep-HPLC to get compound 5 (5.00 g, 95% purity, 41% yield) as a yellow solid.

### Step 3: 5-(3-bromophenyl)-3-fluoro-5,8,8-trimethyl-7,8,9,10-tetrahydrobenzo [b][1,8] naphthyridin-6 (5H)-one (6)

### (S)-5-(3-bromophenyl)-3-fluoro-5,8,8-trimethyl-7,8,9,10-tetrahydrobenzo [b][1,8]naphthyridin-6 (5H)-one (6A)

### (R)-5-(3-bromophenyl)-3-fluoro-5,8,8-trimethyl-7,8,9,10-tetrahydrobenzo [b][1,8] naphthyridin-6 (5H)-one (6B)

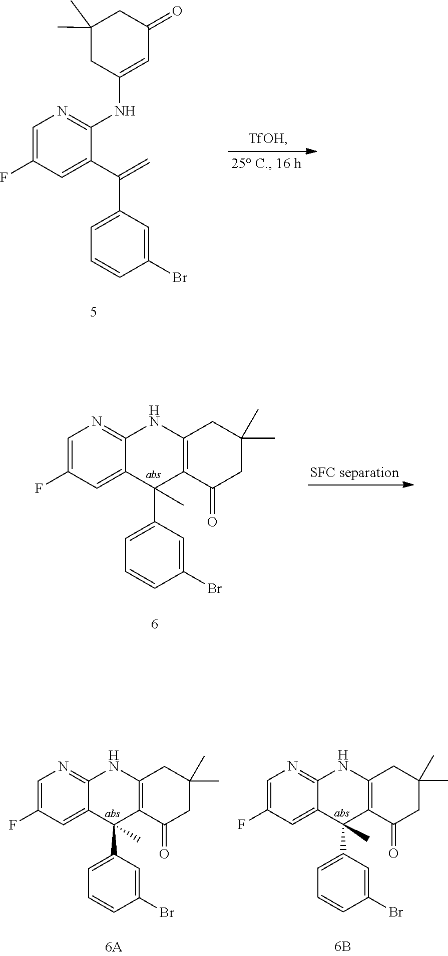 [SMILES: C=C(c1cccc(Br)c1)c1cc(F)cnc1NC1=CC(=O)CC(C)(C)C1 ; CC1(C)CC(=O)C2=C(C1)Nc1ncc(F)cc1C2(C)c1cccc(Br)c1 ; CC1(C)CC(=O)C2=C(C1)Nc1ncc(F)cc1[C@]2(C)c1cccc(Br)c1 ; CC1(C)CC(=O)C2=C(C1)Nc1ncc(F)cc1[C@@]2(C)c1cccc(Br)c1]

To a solution of compound 5 (2.00 g, 4.82 mmol, 1.00 eq) in TfOH (10 mL) was stirred at 25° C. for 16 h. The mixture quenched by 100 ml H₂O and basified to pH=7 with Na₂CO₃. The mixture was filtered, and the filter cake was dried in vacuo. The residue was purified by flash silica gel chromatography (ISCO®; 10 g SepaFlash® Silica Flash Column, Eluent of 30˜35% Ethyl acetate/Petroleum ether gradient @ 100 mL/min) to give compound 6.

Compound 6 was separated by SFC (IC-3S_4_5_40_3ML; Column: Chiralpak IC-3 100×4.6 mm I.D., 3 um; Mobile phase: isopropanol (0.05% DEA) in CO₂ from 5% to 40%; Flow rate: 3 mL/min Wavelength: 220 nm) lyophilization to give:

Peak 1: (S)-5-(3-bromophenyl)-3-fluoro-5,8,8-trimethyl-7,8,9,10-tetrahydrobenzo [b] [1,8]naphthyridin-6 (5H)-one (6A). (700 mg, 98% purity, 34% yield) as a yellow solid.

LCMS: tR=0.504 min., (ES⁺) m/z (M+H)⁺=416.9.

¹H NMR (400 MHz, CD₃OD-d₄) δ=7.97-7.84 (m, 1H), 7.60-7.52 (m, 1H), 7.42-7.34 (m, 1H), 7.28 (br d, J=7.2 Hz, 1H), 7.22-7.14 (m, 1H), 6.97-6.81 (m, 1H), 2.64-2.48 (m, 2H), 2.20-2.04 (m, 3H), 2.00-1.90 (m, 2H), 1.10 (br s, 6H).

Peak 2: (R)-5-(3-bromophenyl)-3-fluoro-5,8,8-trimethyl-7,8,9,10-tetrahydrobenzo [b] [1,8] naphthyridin-6 (5H)-one (6B). (640 mg, 98% purity, 31% yield) as a yellow solid.

LCMS: tR=0.509 min., (ES⁺) m/z (M+H)⁺=416.9.

¹H NMR (400 MHz, CD₃OD-d₄) δ=7.88 (d, J=2.8 Hz, 1H), 7.54 (t, J=2.0 Hz, 1H), 7.37 (td, J=1.6, 8.0 Hz, 1H), 7.27 (ddd, J=1.2, 2.0, 8.0 Hz, 1H), 7.20-7.14 (m, 1H), 6.87 (dd, J=2.8, 9.2 Hz, 1H), 2.59-2.47 (m, 2H), 2.18-2.05 (m, 2H), 2.03 (s, 1H), 1.93 (s, 3H), 1.09 (d, J=2.4 Hz, 6H).

### Example 1.46. (S)-3-fluoro-5-(3-(3-fluoro-5-neopentylpyridin-4-yl)phenyl)-5,8,8-trimethyl-7,8,9,10-tetrahydrobenzo[b][1,8]naphthyridin-6(5H)-one (13)

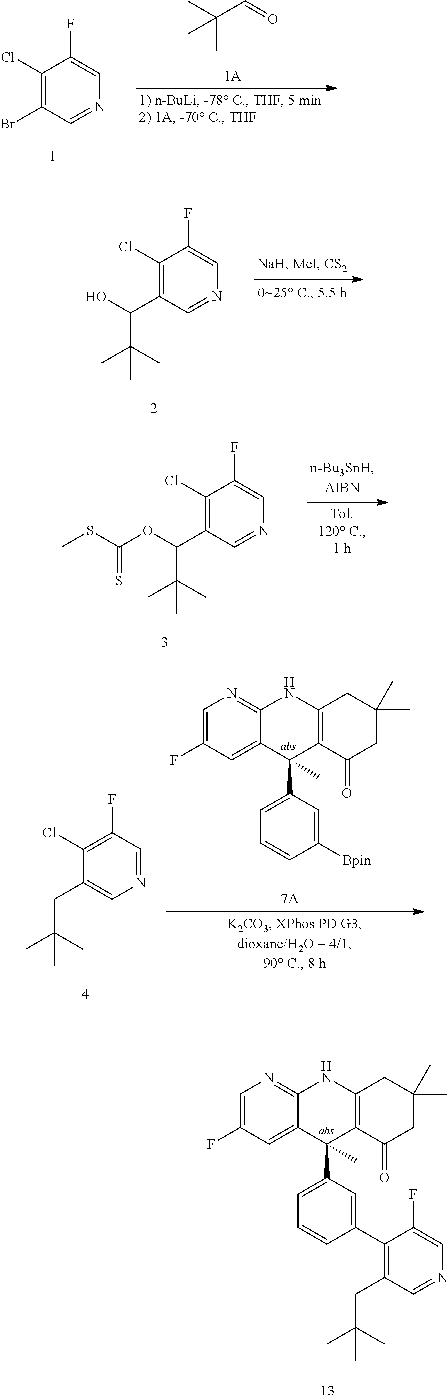

### Step 1: 1-(4-chloro-5-fluoropyridin-3-yl)-2,2-dimethylpropan-1-ol. (2)

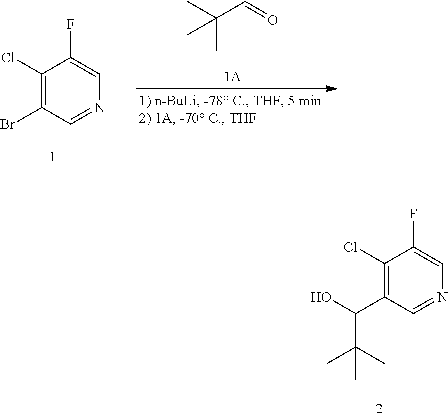

To a solution of 3-bromo-4-chloro-5-fluoro-pyridine (1.00 g, 4.75 mmol, 1 eq) in THF (10 mL) was dropwise to n-BuLi (2.5 M, 2.85 mL, 1.5 eq) under −78° C., stirred 5 min, then 2,2-dimethylpropanal (4.09 g, 47.52 mmol, 10 eq) was dropwise to the reaction mixture. The mixture was stirred at −78° C. for 1 h under N₂. On completion, the reaction mixture was quenched with saturated NH₄Cl (20 mL), and then extracted with EtOAc (20 mL×3). The combined organic layers were washed with brine (20 mL×3), dried over Na₂SO₄, filtered, and concentrated under reduced pressure to give a residue. The residue was purified by column chromatography (SiO₂, Petroleum ether/Ethyl acetate=0/1-5/1) to give compound 2 (800 mg, 77% yield) as a yellow solid.

¹H NMR (400 MHz, DMSO-d₆) δ=8.59 (s, 1H), 8.49 (s, 1H), 5.73 (d, J=4.4 Hz, 1H), 4.74 (d, J=4.4 Hz, 1H), 0.91 (s, 9H).

LCMS: tR=0.351 min., (ES⁺) m/z (M+H)⁺=218.2.

### Step 2: O-(1-(4-chloro-5-fluoropyridin-3-yl)-2,2-dimethylpropyl)S-methyl carbonodithioate (3)

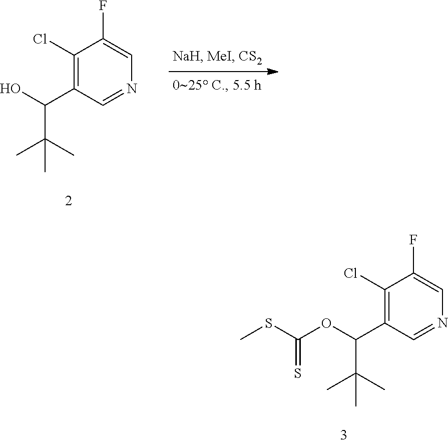 [SMILES: CC(C)(C)C(O)c1cncc(F)c1Cl ; CSC(=S)OC(c1cncc(F)c1Cl)C(C)(C)C]

To a solution of compound 2 (800 mg, 3.68 mmol, 1 eq) in THF (10 mL) was portion wise to NaH (294 mg, 7.35 mmol, 60% purity, 2 eq) at 0° C. under N₂, the mixture was stirred at 25° C. for 0.5 h. The CS₂ (363.80 mg, 4.78 mmol, 1.3 eq) was added to the mixture and stirred at 0° C. for 0.5 h, then MeI (1.04 g, 7.35 mmol, 2 eq) was added dropwise to the mixture. The mixture was stirred at 25° C. for 2 h. On completion, the mixture was quenched by aqueous saturated NH₄Cl to pH=7. The mixture was concentrated under reduced pressure and extracted with EtOAc (10 mL×3) The combined organic layer was washed with brine (10 mL×3), dried over Na₂SO₄, filtered, and concentrated in vacuo. The residue was purified by column chromatography (SiO₂, Petroleum ether/Ethyl acetate=5/1) to give compound 3 (850 mg, 75% yield) as a yellow solid.

¹H NMR (400 MHz, DMSO-d₆) δ=8.70 (s, 1H), 8.40 (s, 1H), 6.40 (s, 1H), 2.63 (s, 3H), 1.04 (s, 9H).

### Step 3: 4-chloro-3-fluoro-5-neopentylpyridine (4)

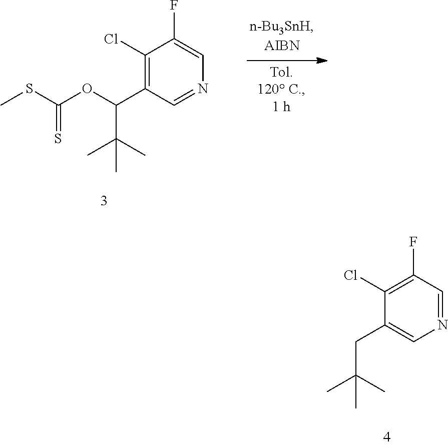 [SMILES: CSC(=S)OC(c1cncc(F)c1Cl)C(C)(C)C ; CC(C)(C)Cc1cncc(F)c1Cl]

To a solution of compound 3 (800 mg, 2.60 mmol, 1 eq) in toluene (8 mL) was purged with nitrogen and heated to 80° C. Then tributylstannane (2.64 g, 9.10 mmol, 3 eq) and AIBN (107 mg, 649 μmol, 0.3 eq) was dissolved in toluene (8 mL) was added dropwise to the mixture. The mixture was stirred at 120° C. for 6 h. On completion, the reaction mixture was quenched with H₂O (20 mL), and then extracted with EtOAc (20 mL×3). The combined organic layers were washed with brine (20 mL×3), dried over Na₂SO₄, filtered, and concentrated under reduced pressure to give a residue. The residue was purified by column chromatography (SiO₂, Petroleum ether/Ethyl acetate=0/1 to 5/1) to give compound 4 (500 mg, 76% yield) as a yellow oil.

LCMS: tR=0.411 min., (ES⁺) m/z (M+H)⁺=186.2.

### Step 4: (S)-3-fluoro-5-(3-(3-fluoro-5-neopentylpyridin-4-yl)phenyl)-5,8,8-trimethyl-7,8,9,10-tetrahydrobenzo[b][1,8]naphthyridin-6(5H)-one (13)

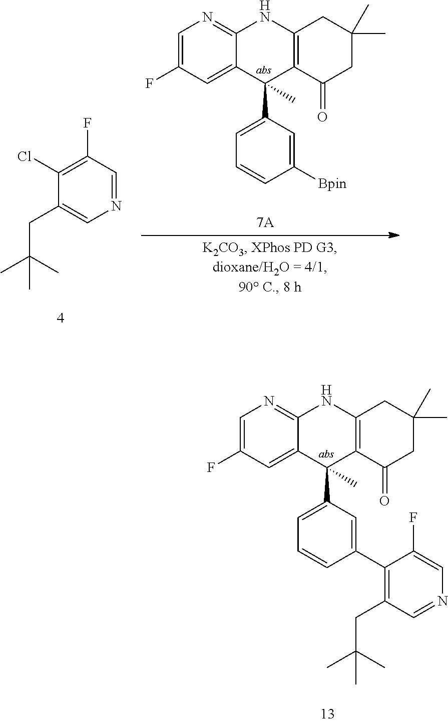

To a solution of compound 4 (80.0 mg, 277 μmol, 1 eq) and compound 7A (192 mg, 416 μmol, 1.5 eq) in dioxane (2 mL) and H₂O (0.5 mL) was added one portion to XPhos Pd G3 (23.50 mg, 27.7 μmol, 0.1 eq) and K₂CO₃ (115 mg, 833 μmol, 3 eq). The mixture was stirred at 90° C. for 8 h under N₂. On completion, the reaction mixture was quenched with saturated NH₄Cl (5 mL), and then extracted with EtOAc (5 mL×3). The combined organic layers were washed with brine (5 mL×3), dried over Na₂SO₄, filtered, and concentrated under reduced pressure to give a residue. The residue was purified by prep-HPLC (column: Welch Ultimate C18 150×25 mm×5 um; mobile phase: [water(FA)-ACN]; B %; 48%-78%, min) to give 5 (8.70 mg, 6% yield) as a yellow solid.

LCMS: tR=0.522 min., (ES⁺) m/z (M+H)⁺=502.2.

¹H NMR (400 MHz, DMSO-d₆) δ=9.93 (s, 1H), 8.40 (d, J=4.8 Hz, 1H), 7.99 (d, J=2.8 Hz, 1H), 7.60 (s, 2H), 7.51-7.34 (m, 5H), 7.14 (dd, J=2.8, 9.6 Hz, 1H), 2.74 (br d, J=3.2 Hz, 3H), 2.10-2.02 (m, 2H), 2.01-1.93 (m, 5H), 1.23 (br s, 1H), 1.01 (br d, J=6.8 Hz 7H), 0.95 (s 10H).

### Example 1.47. (S)(3-(3-(cyclopropylmethyl)-5-fluoropyridin-4-yl)phenyl)-3-fluoro-5,8,8-trimethyl-7,8,9,10-tetrahydrobenzo[b][1,8]naphthyridin-6(5H)-one (14)

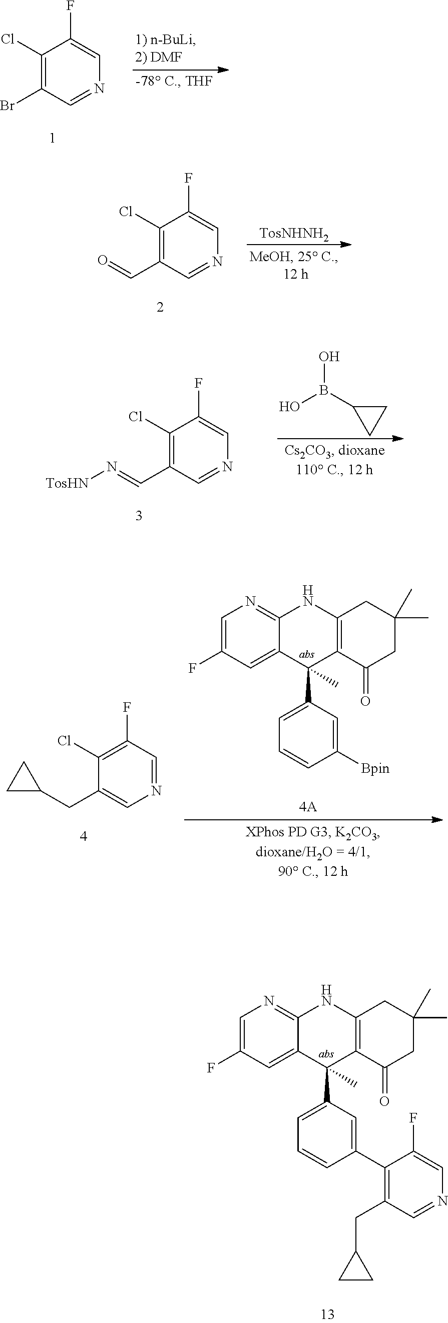

### Step 1: 4-chloro-5-fluoronicotinaldehyde. (2)

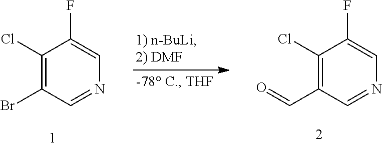 [SMILES: Fc1cncc(Br)c1Cl ; O=Cc1cncc(F)c1Cl]

To a solution of 3-bromo-4-chloro-5-fluoro-pyridine (1.00 g, 4.75 mmol, 1 eq) in THF (10 mL) was added dropwise n-BuLi (2.5 M, 2.85 mL, 1.5 eq) under −78° C. and stirred 5 min, then DMF (6.95 g, 95.0 mmol, 20 eq) was added dropwise to the mixture. The mixture was stirred at −78° C. for 1 h under N₂. On completion, the reaction mixture was quenched with saturated NH₄Cl (20 mL), and then extracted with EtOAc (20 mL×3). The combined organic layers were washed with brine (20 mL×3), dried over Na₂SO₄, filtered, and concentrated under reduced pressure to give a residue. The residue was purified by column chromatography (SiO₂, Petroleum ether/Ethyl acetate=5/1) to give compound 2 (270 mg, 36% yield) as a yellow solid.

¹H NMR (400 MHz, DMSO-d₆) δ=10.29 (s, 1H), 8.98 (d, J=0.8 Hz, 1H), 8.85 (s, 1H).

### Step 2: (E)-N′-((4-chloro-5-fluoropyridin-3-yl)methylene)-4-methylbenzenesulfonohydrazide (3)

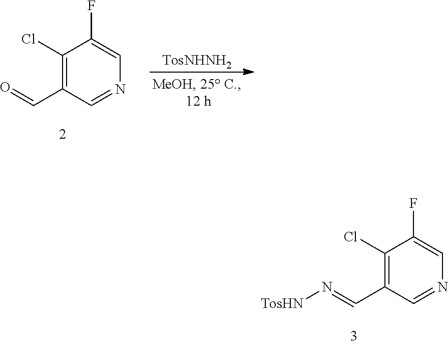

To a solution of compound 2 (250 mg, 1.57 mmol, 1 eq) and 4-methylbenzenesulfonohydrazide (350 mg, 1.88 mmol, 1.2 eq) in MeOH (2.5 mL) was stirred at 25° C. for 12 h. On completion, the reaction mixture was filtered and concentrated under reduced pressure to give a residue. The crude product was triturated with Ethyl acetate (5 mL) to give compound 3 (350 mg, 68% yield) as a yellow solid.

¹H NMR (400 MHz, DMSO-d₆) δ=12.05 (s, 1H), 8.68 (d, J=9.6 Hz, 2H), 8.14 (s, 1H), 7.79 (d, J=8.4 Hz, 2H), 7.43 (d, J=8.4 Hz, 2H), 2.37 (s, 3H).

LCMS: tR=0.393 min., (ES⁺) m/z (M+H)⁺=328.1.

### Step 3: 4-chloro-3-(cyclopropylmethyl)-5-fluoropyridine (4)

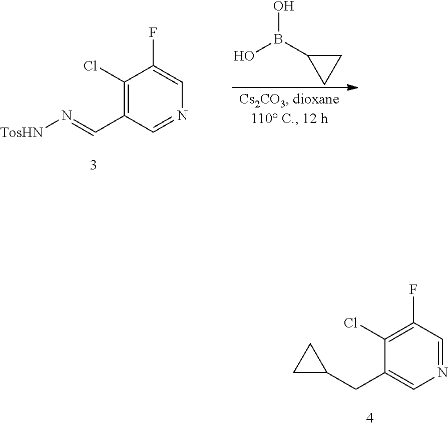

To a solution of compound 3 (300 mg, 915 μmol, 1 eq) and cyclopropylboronic acid (117 mg, 1.37 mmol, 1.5 eq) in dioxane (4.5 mL) was added Cs₂CO₃ (447 mg, 1.37 mmol). The mixture was stirred at 110° C. for 12 h. On completion, the reaction mixture was quenched with saturated NH₄Cl (5 mL), and then extracted with EtOAc (5 mL×3). The combined organic layers were washed with brine (5 mL×3), dried over Na₂SO₄, filtered, and concentrated under reduced pressure to give a residue. The residue was purified by column chromatography (SiO₂, Petroleum ether/Ethyl acetate=10/1) to give compound 4 (25.0 mg, 15% yield) as a yellow solid.

LCMS: tR=0.411 min., (ES⁺) m/z (M+H)⁺=186.2.

### Step 4: (S)-5-(3-(3-(cyclopropylmethyl)-5-fluoropyridin-4-yl)phenyl)-3-fluoro-5,8,8-trimethyl-7,8,9,10-tetrahydrobenzo[b][1,8]naphthyridin-6(5H)-one (14)

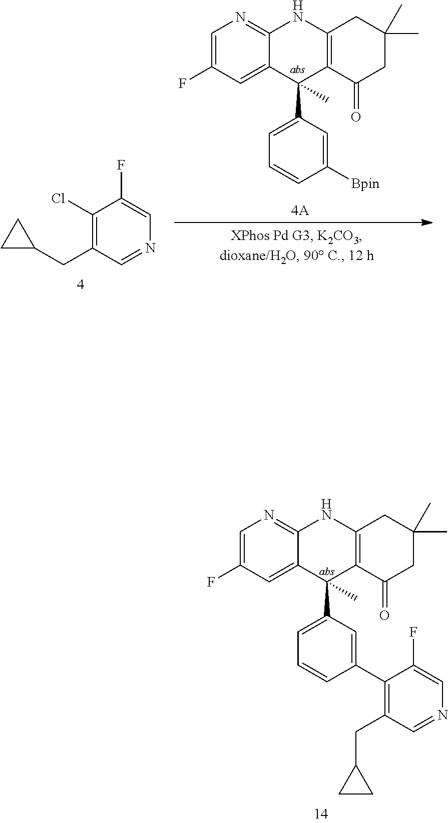

To a solution of compound 4 (25.0 mg, 134 μmol, 1 eq) and compound 4A (62.2 mg, 134 μmol, 1 eq) in dioxane (0.8 mL) and H₂O (0.2 mL) was added XPhos Pd G3 (11.4 mg, 13.4 μmol, 0.1 eq) and K₂CO₃ (55.8 mg, 404 μmol, 3 eq). The mixture was stirred at 90° C. for 12 h. On completion, the reaction mixture was quenched with saturated NH₄Cl (5 mL), and then extracted with EtOAc (5 mL×3). The combined organic layers were washed with brine (5 mL×3), dried over Na₂SO₄, filtered, and concentrated under reduced pressure to give a residue. The residue was purified by prep-HPLC (column: Welch Ultimate C18 150×25 mm×5 um; mobile phase: [water(FA)-ACN]; B %; 48%-78%, min) to give 14 (8.70 mg, 13% yield) as a yellow solid.

LCMS: tR=0.470 min., (ES⁺) m/z (M+H)⁺=486.5.

¹H NMR (400 MHz, DMSO-d₆) δ=9.89 (s, 1H), 8.46 (s, 2H), 7.99 (d, J=2.8 Hz, 1H), 7.49 (d, J=8.4 Hz, 1H), 7.41 (t, J=7.6 Hz, 1H), 7.21 (s, 1H), 7.08 (d, J=7.2 Hz, 1H), 7.03 (dd, J=2.8, 9.6 Hz, 1H), 2.46 (s, 2H), 2.33-2.30 (m, 2H), 2.07-1.97 (m, 2H), 1.95 (s, 3H), 0.99 (d, J=16.0 Hz, 6H), 0.61-0.52 (m, 1H), 0.26-0.21 (m, 2H), 0.13-0.15 (m, 2H).

### Example 1.48. (S)-3-fluoro-5,8,8-trimethyl-5-(3-(1-neopentyl-1H-pyrazol-5-yl)phenyl)-7,8,9,10-tetrahydrobenzo[b][1,8]naphthyridin-6(5H)-one (15)

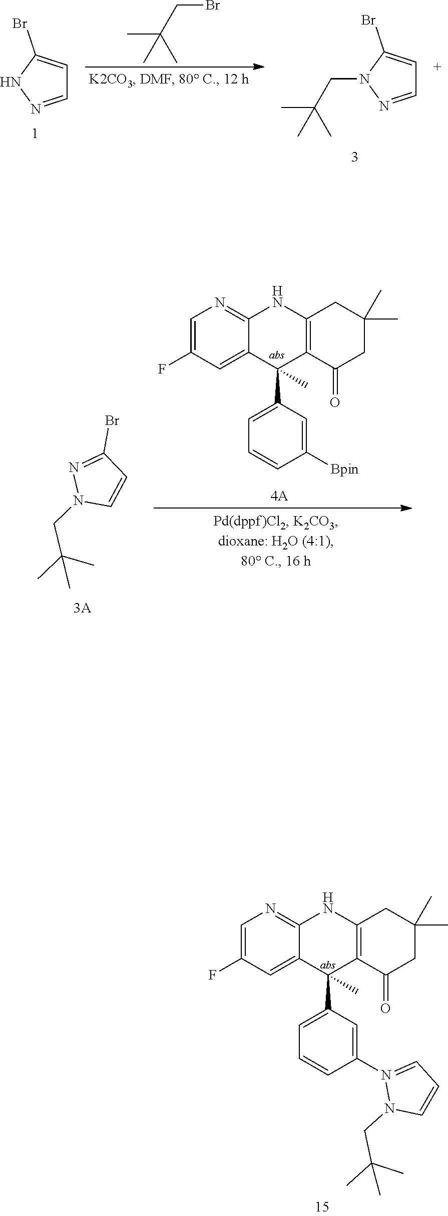

### Step 1: 5-bromo-1-neopentyl-1H-pyrazole (3) & 3-bromo-1-neopentyl-1H-pyrazole (3A)

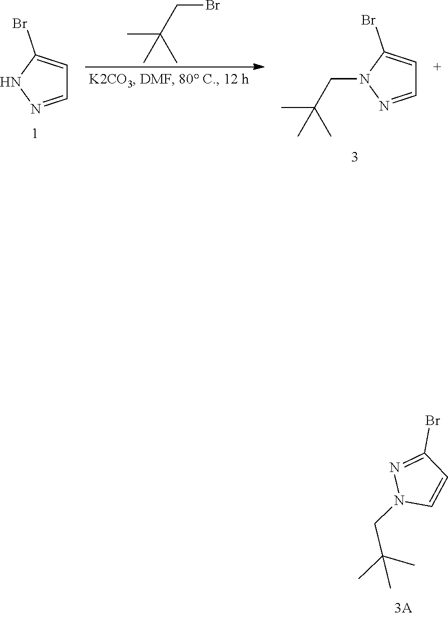

To a solution of compound 1 (5.00 g, 34.0 mmol, 1.00 eq) and 1-bromo-2,2-dimethylpropane (7.71 g, 51.0 mmol, 1.50 eq) in DMF (50 mL) was added K₂CO₃ (14.11 g, 102 mmol, 3.00 eq) stirred at 80° C. for 12 h. On completion, the reaction was diluted with H₂O (60 mL), extracted with DCM (3×60 mL), washed with saturated NaCl (90 mL), dried over Na₂SO₄, filtered, and concentrated under reduced pressure to give a residue. The yellow residue was purified by column chromatography (SiO₂, Petroleum ether:Ethyl acetate=10:1-3:1-0:1) to give compound 3 & 3A (1.7 g, mixture) as a yellow oil.

¹H NMR (400 MHz, DMSO-d₆) δ=7.69 (d, J=2.4 Hz, 1H), 7.55 (d, J=2.0 Hz, 1H), 6.42 (d, J=2.0 Hz, 1H), 6.35 (d, J=2.4 Hz, 1H), 3.93 (s, 1H), 3.88 (s, 2H), 0.93 (s, 3H), 0.87 (s, 9H).

### Step 2: (S)-3-fluoro-5,8,8-trimethyl-5-(3-(1-neopentyl-1H-pyrazol-5-yl)phenyl)-7,8,9,10-tetrahydrobenzo[b][1,8]naphthyridin-6(5H)-one (15)

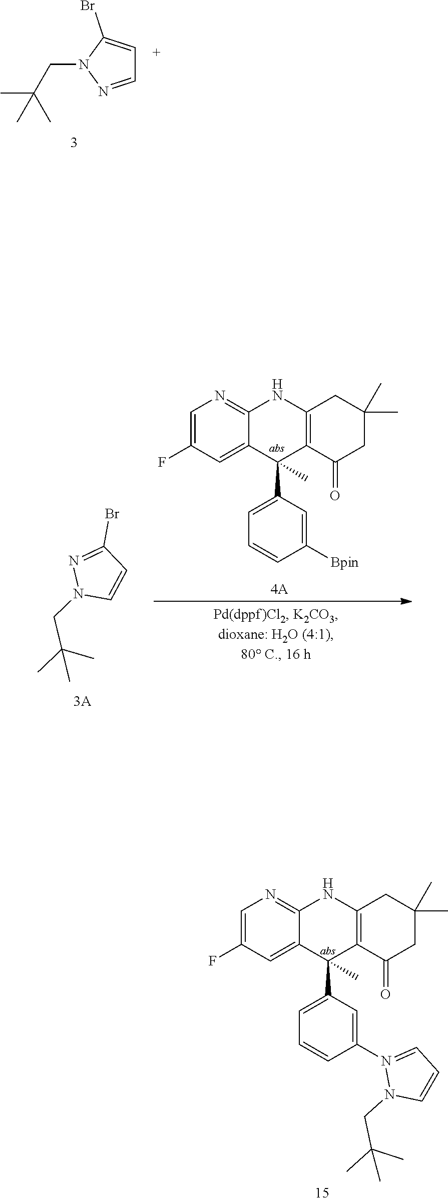

To a mixture of compound 3& 3A (300 mg, 0.65 mmol, 1.00 eq) and 4A (141 mg, 0.65 mmol, 1.00 eq) in 1,4-dioxane (4 mL) and H₂O (1 mL) was added K₂CO₃ (269 mg, 1.95 mmol, 3.00 eq) and Pd(dppf)Cl₂·CH₂Cl₂ (53 mg, 0.06 mmol, 0.100 eq) in one portion under N₂, the mixture was stirred under N₂ at 80° C. for 16 h. On completion, the reaction was diluted with water (10 mL), extracted with EtOAc (3×10 mL), washed with saturated NaCl (15 mL), dried over Na₂SO₄, filtered, and concentrated under reduced pressure to give a residue. The residue was purified by prep-HPLC (Column: Phenomenex luna C18 150×25 mm×10 um, water (0.1% FA)-ACN, 59-80% ACN, 2 min) to give compound 15 (26 mg, 97% purity, 8% yield, confirmed by 2D NMR) as a yellow solid.

LCMS: tR=0.598 min., (ES⁺) m/z (M+H)⁺=473.4.

¹H NMR (400 MHz, DMSO-de) δ=9.92 (br s, 1H), 7.99 (d, J=2.8 Hz, 1H), 7.50-7.42 (m, 2H), 7.41-7.34 (m, 1H), 7.29 (s, 1H), 7.13 (br d, J=7.6 Hz, 1H), 7.02 (dd, J=2.4, 9.6 Hz, 1H), 6.25 (d, J=1.6 Hz, 1H), 3.95-3.76 (m, 2H), 2.54 (br s, 1H), 2.47-2.42 (m, 1H), 2.07-1.97 (m, 2H), 1.95 (s, 3H), 1.00 (d, J=9.4 Hz, 6H), 0.59 (s, 9H).

### Example 1.49. (S)-5-(3-(3-fluoro-5-methoxypyridin-4-yl)phenyl)-5,8,8-trimethyl-3-(trifluoromethyl)-5,8,9,10-tetrahydrobenzo[b][1,8]naphthyridin-6(7H)-one (16A)

### (R)-5-(3-(3-fluoro-5-methoxypyridin-4-yl)phenyl)-5,8,8-trimethyl-3-(trifluoromethyl)-5,8,9,10-tetrahydrobenzo[b][1,8]naphthyridin-6(7H)-one (16B)

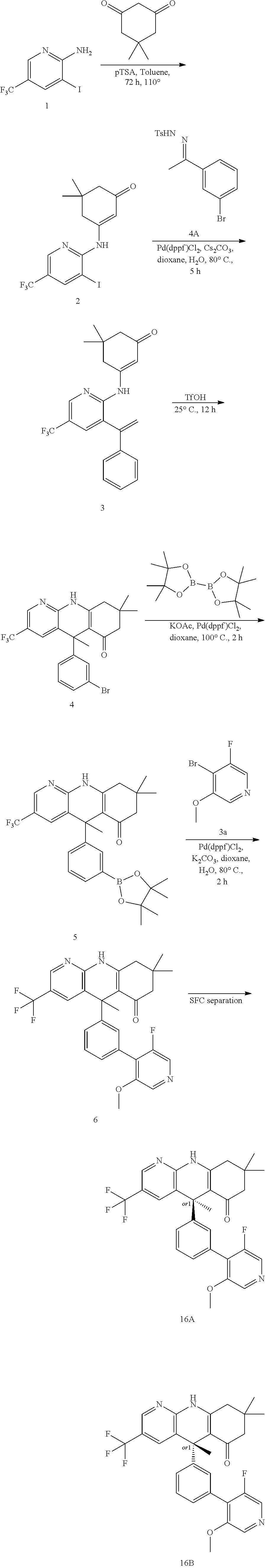

### Step 1: 3-((3-iodo-5-(trifluoromethyl)-2-yl)amino)-5,5-dimethylcyclohex-2-en-1-one. (2)

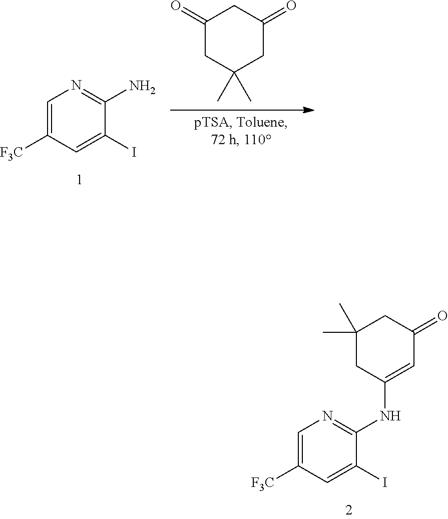

To a solution 3-iodo-5-(trifluoromethyl)pyridin-2-amine (15.00 g, 52.1 mmol, 1.00 eq) and 5,5-dimethylcyclohexane-1,3-dione (7.30 g, 52.1 mmol, 1.0) eq) in Toluene (30 mL) was added pTSA (896 mg, 0.1 eq) portion wise was stirred at 110° C. for 72 h. On completion, the mixture quenched by 20 mL H₂O and basified to pH=7 with Na₂CO₃. The mixture was filtered, and the filter cake was dried under vacuum. The residue was purified by flash silica gel chromatography (ISCO®; 10 g SepaFlash® Silica Flash Column, Eluent of 0˜10% Ethyl acetate/Petroleum ether gradient @ 60 mL/min) to give compound 2 (4.00 g, 17% yield) as a yellow solid.

LCMS: tR=0.479 min., (ES⁺) m/z (M+H)⁺=411.6.

### Step 2: 3-((3-(1-(3-bromophenyl)vinyl)-5-(trifluoromethyl)pyridin-2-yl)amino)-5,5-dimethylcyclohex-2-enone (3)

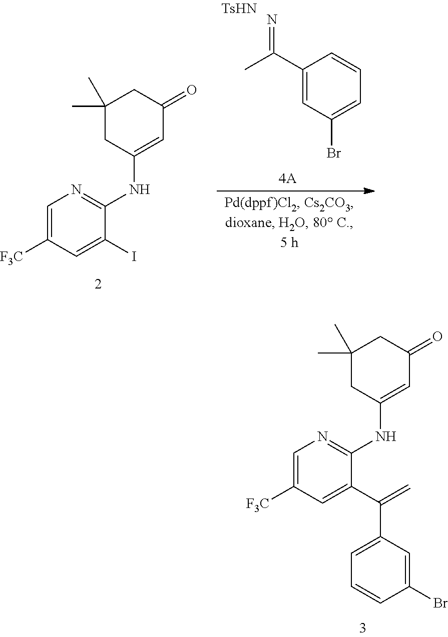

To a solution of compound 2 (3 g, 7.31 mmol, 1 eq) and N-[(E)-1-(3-bromophenyl)ethylideneamino]-4-methyl-benzenesulfonamide (2.95 g, 8.05 mmol, 1.1 eq) in dioxane (30 mL) and H₂O (30 mL) was added Pd(dppf)Cl₂ (0.53 g, 0.73 mmol, 0.1 eq) and Cs₂CO₃ (7.13 g, 21.9 mmol, 3 eq) portion wise with the temperature kept at 25° C. under N₂ to get a yellow solution. The reaction mixture was stirred at 80° C. for 5 h. On completion, the reaction mixture was quenched with NH₄Cl (30 mL), and then extracted with EtOAc (30 mL×3). The combined organic layers were washed with brine (50 mL×3), dried over Na₂SO₄, filtered, and concentrated under reduced pressure to give a residue. The residue was purified by flash silica gel chromatography to give compound 3 (600 mg, 18% yield) as a yellow solid.

LCMS: tR=0.588 min., (ES⁺) m/z (M+H)⁺=466.9.

### Step 3: 5-(3-bromophenyl)-5,8,8-trimethyl-3-(trifluoromethyl)-7,8,9,10-tetrahydrobenzo [b][1,8] naphthyridin-6(5H)-one (5)

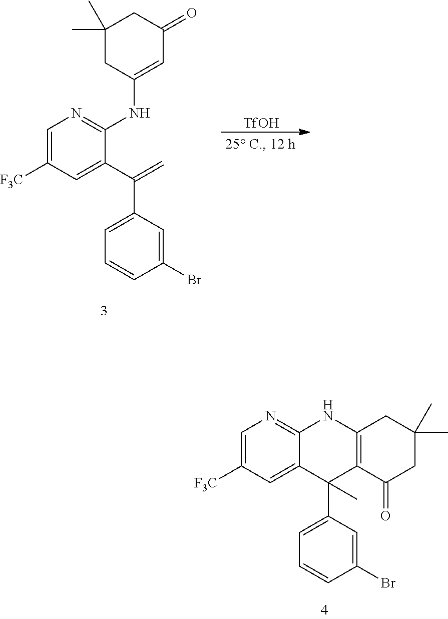

To a solution of compound 3 (300 mg, 644 μmol, 1 eq) was added TfOH (5.10 g, 33.9 mmol, 3 mL). The mixture was stirred at 25° C. for 12 h. On completion, the reaction mixture was quenched with aq. NaOH (15 mL), and then extracted with EtOAc (15 mL×3). The combined organic layers were washed with brine (15 mL×3), dried over Na₂SO₄, filtered, and concentrated under reduced pressure to give a residue. The residue was purified by column chromatography (SiO₂, Petroleum ether/Ethyl acetate=3/1) to give compound 4 (270 mg, 72% yield) as a yellow solid.

¹H NMR (400 MHz, DMSO-d₆) δ=8.86 (s, 1H), 8.21 (d, J=2.0 Hz, 2H), 5.30 (br d, J=3.2 Hz, 1H), 4.45 (br s, 1H), 3.81 (s, 3H), 1.21 (s, 10H), 0.81 (s, 9H), 2.13-1.28 (m, 1H).

### Step 4: 5,8,8-trimethyl-5-[3-(4,4,5,5-tetramethyl-1,3,2-dioxaborolan-2-yl)phenyl]-3-(trifluoromethyl)-9,10-dihydro-7H-benzo[b][1,8]naphthyridin-6-one (5)

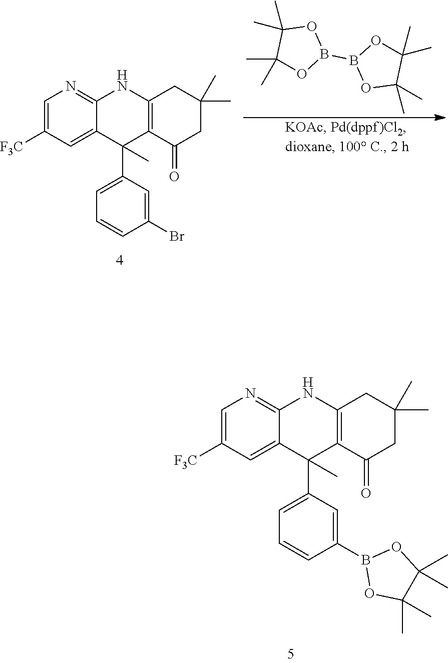

To a solution of 4,4,5,5-tetramethyl-2-(4,4,5,5-tetramethyl-1,3,2-dioxaborolan-2-yl)-1,3,2-dioxaborolane (546 mg, 2.15 mmol, 2 eq) and compound 4 (500 mg, 1.07 mmol, 1.00 eq) in dioxane (5 mL) was added Pd(dppf)Cl₂ (79 mg, 0.11 mmol, 0.1 eq) and KOAc (316 mg, 3.22 mmol, 3 eq) portion wise with the temperature kept at 25° C. under N₂ to get a yellow solution. The reaction mixture was stirred at 100° C. for 2 hours. On completion, the reaction mixture was poured into saturated NH₄Cl aqueous solution (10 mL) and extracted with EtOAc (10 mL×3). The combined organic layer was washed with brine (10 mL), dried over Na₂SO₄, filtered, and concentrated. The yellow residue was purified by column chromatography (SiO₂, Petroleum ether/Ethyl acetate=20:1-5:1) to give 7 (400 mg, 73% yield) as a yellow solid.

¹H NMR (400 MHz, DMSO-d6) δ=10.28 (s, 1H), 8.36 (s, 1H), 7.66 (s, 1H), 7.54 (br d, J=8.0 Hz, 1H), 7.42 (d, J=7.2 Hz, 1H), 7.33-7.24 (m, 2H), 3.93 (s, 2H), 1.95 (s, 4H), 1.27 (d, J=7.6 Hz, 12H), 1.17 (s, 3H), 1.08 (s, 13H), 1.03 (d, J=5.2 Hz, 6H).

### Step 5: 5-(3-(3-fluoro-5-methoxypyridin-4-yl)phenyl)-5,8,8-trimethyl-3-(trifluoromethyl)-5,8,9,10-tetrahydrobenzo[b][1,8]naphthyridin-6(7H)-one (6)

### (S)-5-(3-(3-fluoro-5-methoxypyridin-4-yl)phenyl)-5,8,8-trimethyl-3-(trifluoromethyl)-7,8,9,10-tetrahydrobenzo[b][1,8]naphthyridin-6(5H)-one (16A) & (R)-5-(3-(3-fluoro-5-methoxypyridin-4-yl)phenyl)-5,8,8-trimethyl-3-(trifluoromethyl)-7,8,9,10-tetrahydrobenzo[b][1,8]naphthyridin-6(5H)-one (16B)

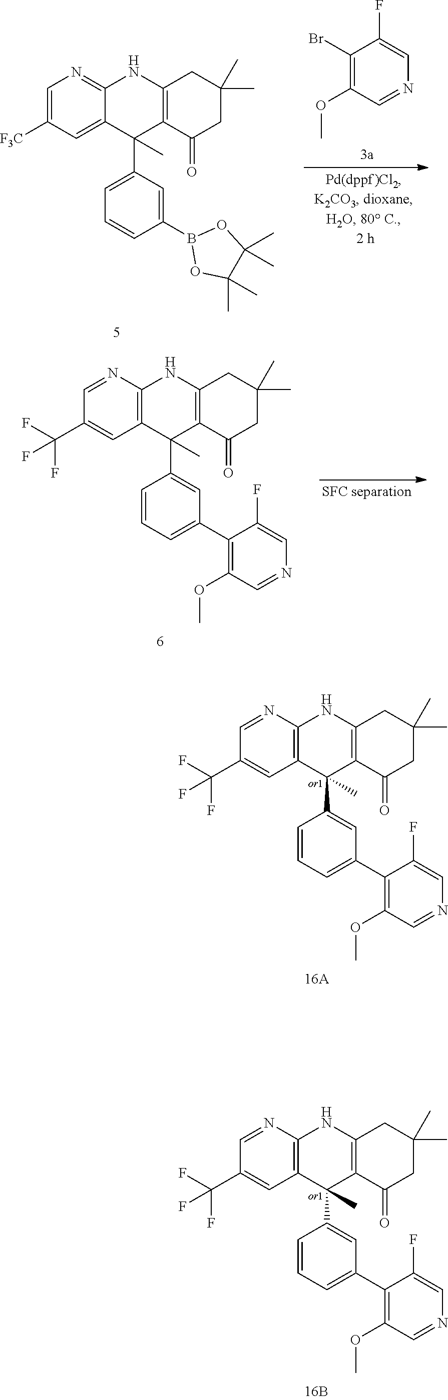

To a solution of compound 3a (80 mg, 0.390 mmol, 1.00 eq) and compound 5 (200 mg, 0.39 mmol, 1.00 eq) in dioxane (1 mL) and H₂O (1 mL) was added Pd(dppf)Cl₂ (29 mg, 0.04 mmol, 0.1 eq) and K₂CO₃ (162 mg, 1.17 mmol, 3 eq) portion wise with the temperature kept at 25° C. under N₂ to get a yellow solution. The reaction mixture was stirred at 80° C. for 2 hours. On completion, the reaction mixture was quenched with saturated NH₄Cl (5 mL), and then extracted with EtOAc (5 mL×3). The combined organic layers were washed with brine (5 mL×3), dried over Na₂SO₄, filtered, and concentrated under reduced pressure to give a residue. The crude product was purified by re-MPLC (0.1% FA, 5˜100% ACN/H₂O) to give compound 6 (60 mg, 30% yield) as a yellow solid.

LCMS: tR=0.511 min., (ES⁺) m/z (M+H)⁺=512.2.

¹H NMR (400 MHz, DMSO-d₆) δ=10.27 (s, 1H), 8.35 (d, J=10.8 Hz, 3H), 7.46-7.36 (m, 3H), 7.33 (d, J=1.6 Hz, 1H), 7.19 (br d, J=7.2 Hz, 1H), 3.82 (s, 3H), 2.54 (br s, 2H), 2.05 (d, J=3.6 Hz, 2H), 1.94 (s, 3H), 1.02 (d, J=3.2 Hz, 6H).

The product was separated by SFC ((S,S)Whelk-O1 50×4.6 mm I.D., 1.8 um; mobile phase: [0.05% DEA MeOH]; B %; 5%-40%, B5.5; 180 min) to give:

Peak 1: (S)-5-(3-(3-fluoro-5-methoxypyridin-4-yl)phenyl)-5,8,8-trimethyl-3-(trifluoromethyl)-7,8,9,10-tetrahydrobenzo[b][1,8]naphthyridin-6(5H)-one (16A) (23 mg, 35% yield).

LCMS: tR=0.480 min., (ES⁺) m/z (M+H)⁺=512.5.

¹H NMR (400 MHz, DMSO-d₆) δ=10.27 (s, 1H), 8.35 (d, J=10.8 Hz, 3H), 7.46-7.36 (m, 3H), 7.33 (d, J=1.6 Hz, 1H), 7.19 (br d, J=7.2 Hz, 1H), 3.82 (s, 3H), 2.54 (br s, 2H), 2.05 (d, J=3.6 Hz, 2H), 1.94 (s, 3H), 1.02 (d, J=3.2 Hz, 6H).

Peak 2: (R)-5-(3-(3-fluoro-5-methoxypyridin-4-yl)phenyl)-5,8,8-trimethyl-3-(trifluoromethyl)-7,8,9,10-tetrahydrobenzo[b][1,8]naphthyridin-6(5H)-one (16A) (20 mg, 31% yield).

LCMS: tR=0.476 min., (ES⁺) m/z (M+H)⁺=512.5.

¹H NMR (400 MHz, DMSO-d₆) δ=10.27 (s, 1H), 8.35 (d, J=10.8 Hz, 3H), 7.45-7.35 (m, 3H), 7.32 (s, 1H), 7.18 (br d, J=7.6 Hz, 1H), 3.82 (s, 3H), 2.58-2.53 (m, 2H), 2.05 (br d, J=3.2 Hz, 2H), 1.94 (s, 3H), 1.02 (br d, J=3.6 Hz, 6H).

### Example 1.50. (S)-5,8,8-trimethyl-3-(trifluoromethyl)-5-(3-(1,3,5-trimethyl-1H-pyrazol-4-yl)phenyl)-7,8,9,10-tetrahydrobenzo[b][1,8]naphthyridin-6(5H)-one (17A)

### (R)-5,8,8-trimethyl-3-(trifluoromethyl)-5-(3-(1,3,5-trimethyl-1H-pyrazol-4-yl)phenyl)-7,8,9,10-tetrahydrobenzo[b][1,8]naphthyridin-6(5H)-one (17B)

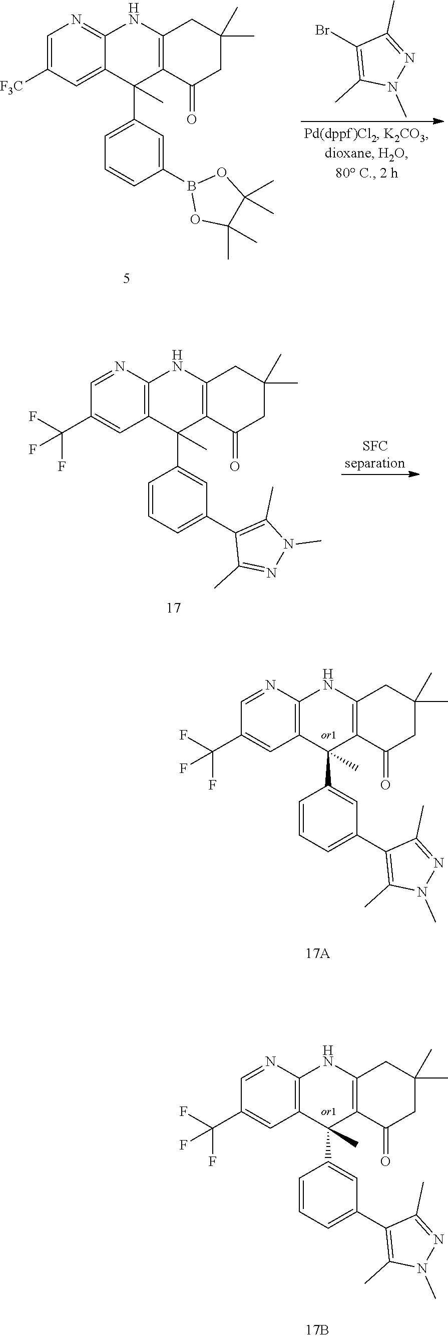

To a solution of compound 5 (150 mg, 292 μmol, 1 eq) and 4-bromo-1,3,5-trimethyl-pyrazole (83.0 mg, 439 μmol, 1.5 eq) in dioxane (0.4 mL) and H₂O (0.1 mL) was added Pd(dppf)Cl₂ (21.4 mg, 29.2 μmol, 0.1 eq) and K₂CO₃ (121 mg, 878 μmol, 3 eq). The mixture was stirred at 80° C. for 2 h. On completion, the reaction mixture was quenched with saturated NH₄Cl (5 mL), and then extracted with EtOAc (5 mL×3). The combined organic layers were washed with brine (5 mL×3), dried over Na₂SO₄, filtered, and concentrated under reduced pressure to give a residue. The crude product was purified by reversed phase HPLC (0.1% FA condition, 55% ACN/H2O) to give compound 6 (50 mg, 40% yield) as a yellow solid.

The product compound 6 (50 mg) was separated by SFC (B\AD)-5-40-3 ML-35T.l cm; Column: Chiralpak AS-3 50×4.6 mm I.D. 3 um; Mobile phase: Phase A for CO₂, and Phase B for IPA (0.05% DEA); Gradient elution: B in A from 5% to 40% Flow rate: 3 mL/min; Detector: PDA; Column Temp: 35 C; Back Pressure: 100 Bar) to give:

Peak 1: (S)-5,8,8-trimethyl-3-(trifluoromethyl)-5-(3-(1,3,5-trimethyl-1H-pyrazol-4-yl)phenyl)-5,8,9,10-tetrahydrobenzo[b][1,8]naphthyridin-6(7H)-one (17A) (22.3 mg, 45% yield).

LCMS: tR=0.447 min., (ES⁺) m/z (M+H)⁺=495.4.

¹H NMR (400 MHz, DMSO-d₆) δ=10.26 (s, 1H), 8.35 (s, 1H), 7.36-7.23 (m, 3H), 7.18 (s, 1H), 6.99 (br d, J=7.2 Hz, 1H), 3.67 (s, 3H), 2.45 (br s, 2H), 2.14 (s, 3H), 2.04 (s, 5H), 1.96 (s, 3H), 1.31-1.17 (m, 3H), 1.01 (br d, J=6.8 Hz, 6H).

Peak 2: (R)-5,8,8-trimethyl-3-(trifluoromethyl)-5-(3-(1,3,5-trimethyl-1H-pyrazol-4-yl)phenyl)-5,8,9,10-tetrahydrobenzo[b][1,8]naphthyridin-6(7H)-one (17B) (18.6 mg, 37% yield).

LCMS: tR=0.633 min., (ES⁺) m/z (M+H)⁺=495.3.

¹H NMR (400 MHz, DMSO-d₆) δ=10.26 (s, 1H), 8.35 (s, 1H), 7.34 (d, J=1.6 Hz, 1H), 7.31-7.24 (m, 2H), 7.19-7.16 (m, 1H), 6.99 (br d, J=7.2 Hz, 1H), 3.67 (s, 3H), 2.14 (s, 3H), 2.04 (s, 5H), 1.96 (s, 3H), 1.23 (s, 2H), 1.01 (d, J=6.8 Hz, 6H).

### Example 1.51. 5-(3-(1-isopropyl-1H-imidazol-5-yl)phenyl)-5,8,8-trimethyl-3-(trifluoromethyl)-5,8,9,10-tetrahydrobenzo[b][1,8]naphthyridin-6(7H)-one (18), (S)-5-(3-(1-isopropyl-1H-imidazol-5-yl)phenyl)-5,8,8-trimethyl-3-(trifluoromethyl)-7,8,9,10-tetrahydrobenzo[b][1,8]naphthyridin-6(5H)-one (18A), & (R)-5-(3-(1-isopropyl-1H-imidazol-5-yl)phenyl)-5,8,8-trimethyl-3-(trifluoromethyl)-7,8,9,10-tetrahydrobenzo[b][1,8]naphthyridin-6(5H)-one (18B)

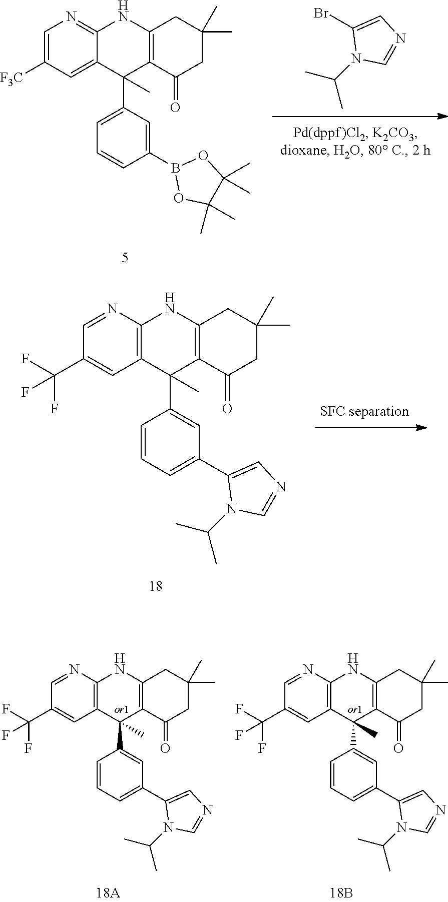

To a solution of compound 5 (300 mg, 0.59 mmol, 1.00 eq) and 5-bromo-1-isopropyl-1H-imidazole (133 mg, 0.70 mmol, 1.20 eq) in 1,4-dioxane (2 mL) and H₂O (2 mL) was added Pd(dppf)Cl₂ (47 mg, 0.059 mmol, 0.10 eq) and K₂CO₃ (243 mg, 1.76 mmol, 3.00 eq) stirred under N₂, the mixture was stirred under N2 at 80° C. for 2 h. On completion, the reaction was concentrated under reduced pressure to give a residue. The crude product was purified by re-MPLC to give compound 18 (130 mg).

### Step 2: (S)-5-(3-(1-isopropyl-1H-imidazol-5-yl)phenyl)-5,8,8-trimethyl-3-(trifluoromethyl)-7,8,9,10-tetrahydrobenzo[b][1,8]naphthyridin-6(5H)-one (18A)

### (R)-5-(3-(1-isopropyl-1H-imidazol-5-yl)phenyl)-5,8,8-trimethyl-3-(trifluoromethyl)-7,8,9,10-tetrahydrobenzo[b][1,8]naphthyridin-6(5H)-one (18B)

Compound 8 (130 mg) was separated by SFC ((S,S)Whelk-O1-MeOH (DEA)-5-40-3 mL-35TB.l cm; Column: (S,S)Whelk-O1 50×4.6 mm I.D., 3.5 um Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA; Column Temp: 35 C; Back Pressure: 100 Bar) to give:

Peak 1: (S)-5-(3-(1-isopropyl-1H-imidazol-5-yl)phenyl)-5,8,8-trimethyl-3-(trifluoromethyl)-7,8,9,10-tetrahydrobenzo[b][1,8]naphthyridin-6(5H)-one (18A) (22 mg, 98% purity, 7% yield).

LCMS: tR=0.371 min., (ES+) m/z (M+H)+=495.4.

¹H NMR (400 MHz, DMSO-d6) δ=10.30 (br s, 1H), 8.36 (d, J=1.2 Hz, 1H), 7.88 (s, 1H), 7.44-7.39 (m, 1H), 7.39-7.34 (m, 1H), 7.33-7.27 (m, 2H), 7.13 (d, J=7.2 Hz, 1H), 6.88 (s, 1H), 4.18 (td, J=6.8, 13.2 Hz, 1H), 2.57-2.52 (m, 1H), 2.46 (s, 1H), 2.13-2.00 (m, 2H), 1.99-1.91 (m, 3H), 1.29 (dd, J=6.8, 18.0 Hz, 6H), 1.01 (d, J=9.2 Hz, 6H).

Peak 2: (R)-5-(3-(1-isopropyl-1H-imidazol-5-yl)phenyl)-5,8,8-trimethyl-3-(trifluoromethyl)-7,8,9,10-tetrahydrobenzo[b][1,8]naphthyridin-6(5H)-one (18B) (25 mg, 97% purity, 8% yield).

LCMS: tR=0.367 min., (ES+) m/z (M+H)+=495.7.

¹H NMR (400 MHz, DMSO-d6) δ=10.28 (br s, 1H), 8.37 (ci, J=0.8 Hz, 1H), 7.88 (s, 1H), 7.45-7.39 (m, 11H), 7.39-7.34 (m, 1H), 7.33-7.27 (m, 2H), 7.13 (ci, J=7.2 Hz, 1H), 6.88 (s, 1H), 4.18 (td, J=6.8, 13.2 Hz, 1H), 2.57-2.52 (nm, 1H), 2.46 (s, 1H), 2.03 (d, J=10.4 Hz, 2H), 1.99-1.95 (mi, 3H), 1.29 (dci, J=6.8, 17.6 Hz, 6H), 1.01 (d, J=9.6 Hz, 6H).

### Example 1.52. (S)-5(3-(3-fluoro-5-methoxypyridin-4-yl)phenyl)-5,8,8-trimethyl-3-neopentyl-5,8,9,10-tetrahydrobenzo[b][1,8]naphthyridin-6(7H)-one (19A)

### (R)-5-(3-(3-fluoro-5-methoxypyridin-4-yl)phenyl)-5,8,8-trimethyl-3-neopentyl-5,8,9,10-tetrahydrobenzo[b][1,8]naphthyridin-6(7H)-one (19B)

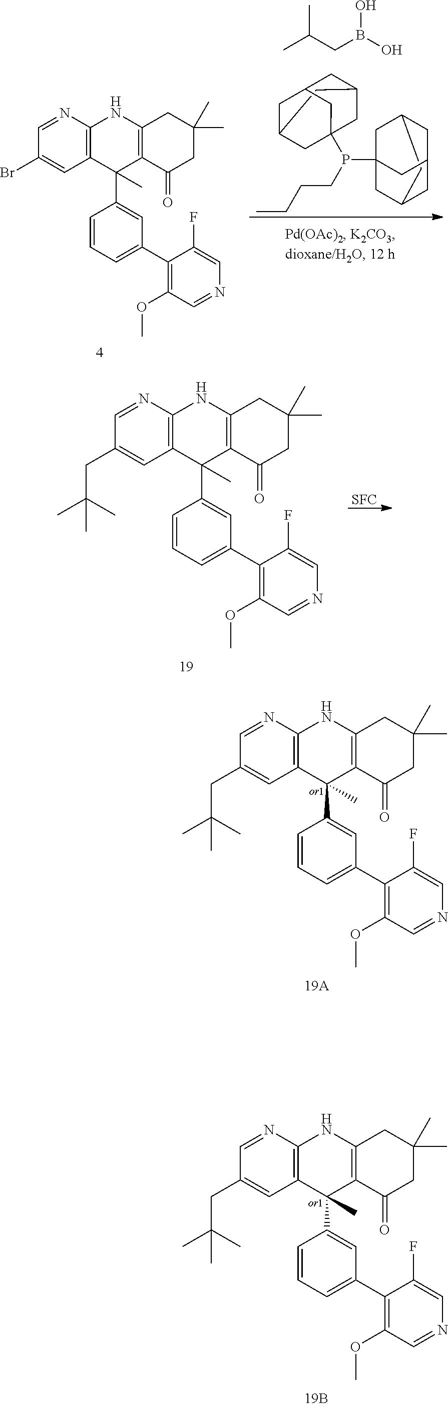

To a suspension of compound 4 (0.20 g, 0.38 μmol, 1.00 eq), K₂CO₃ (141 mg, 1.15 mmol, 3.00 eq) and 2,2-dimethylpropylboronic acid (444 mg, 3.83 mmol, 10.0 eq) in dioxane (1.6 mL) and H₂O (0.4 mL) was added acetoxy(1-oxoethoxy)palladium (5 mg, 19.1 μmol, 0.05 eq) and bis(1-adamantyl)-butyl-phosphane (14 mg, 38.3 mmol, 0.1 eq) portion wise with the temperature kept at 25° C. under N₂ to get a yellow solution. The reaction mixture was stirred at 100° C. for 24 h. On completion, the reaction mixture was poured into 5 wt. % NH₃—H₂O aqueous solution (10 mL) and extracted with EtOAc (50 mL×3). The combined organic layer was washed with brine (10 mL), dried over Na₂SO₄, filtered, concentrated. The residue was purified by prep-HPLC (column: Welch Xtimate C18 150×25 mm×5 um, mobile phase: [water(0.05% NH₃·H₂O)-ACN]; B %; 24%-54%, 10 mm) to give compound 2 (40 mg, 20% yield).

The product was separated by SFC (Column: Chiralpak AD-3 50×4.6 mm I.D., 3 um); Mobile phase: Phase A for CO₂, and Phase B for IPA (0.05% DEA); Gradient elution: B in A from 5% to 40%; Flow rate: 3 mL/min Wavelength: 220 nm) to give:

Peak 1: (S)-5-(3-(3-fluoro-5-methoxypyridin-4-yl)phenyl)-5,8,8-trimethyl-3-neopentyl-5,8,9,10-tetrahydrobenzo[b][1,8]naphthyridin-6(7H)-one (19A) (12.1 mg, 30% yield).

LCMS: tR=0.490 min., (ES⁺) m/z (M+H)⁺=514.3.

¹H NMR (400 MHz, DMSO-d₆) δ=9.73 (s, 1H), 8.33 (d, J=13.2 Hz, 2H), 7.72 (d, J=2.0 Hz, 1H), 7.43-7.39 (m, 1H), 7.34-7.30 (m, 2H), 7.12 (d, J=7.2 Hz, 1H), 6.81 (d, J=1.6 Hz, 1H), 3.82 (s, 3H), 2.47 (br d, J=1.6 Hz, 2H), 2.25-2.17 (m, 2H), 2.07-1.95 (m, 2H), 1.90 (s, 3H), 1.01 (d, J=5.6 Hz, 6H), 0.64 (s, 9H).

Peak 2: (R)-5-(3-(3-fluoro-5-methoxypyridin-4-yl)phenyl)-5,8,8-trimethyl-3-neopentyl-5,8,9,10-tetrahydrobenzo[b][1,8]naphthyridin-6(7H)-one (19B) (16.5 mg, 41% yield).

LCMS: tR=0.496 min., (ES⁺) m/z (M+H)⁺=514.3.

¹H NMR (400 MHz, DMSO-d₆) δ=9.73 (s, 1H), 8.33 (d, J=13.2 Hz, 2H), 7.72 (d, J=1.6 Hz, 1H), 7.42-7.39 (m, 1H), 7.35-7.30 (m, 2H), 7.12 (d, J=7.6 Hz, 1H), 6.81 (d, J=1.6 Hz, 1H), 3.82 (s, 3H), 2.48 (br d, J=1.2 Hz, 2H), 2.25-2.18 (m, 2H), 2.01 (d, J=15.2 Hz, 2H), 1.90 (s, 3H), 1.01 (d, J=5.8 Hz, 6H), 0.64 (s, 9H).

### Example 1.53. (S)-5,8,8-trimethyl-5-phenyl-3-(trifluoromethyl)-7,8,9,10-tetrahydropyrido[2,3-b][1,6]naphthyridin-6(5H)-one (67A)

### (R)-5,8,8-trimethyl-5-phenyl-3-(trifluoromethyl)-7,8,9,10-tetrahydropyrido[2,3-b][1,6]naphthyridin-6(5H)-one (6B)

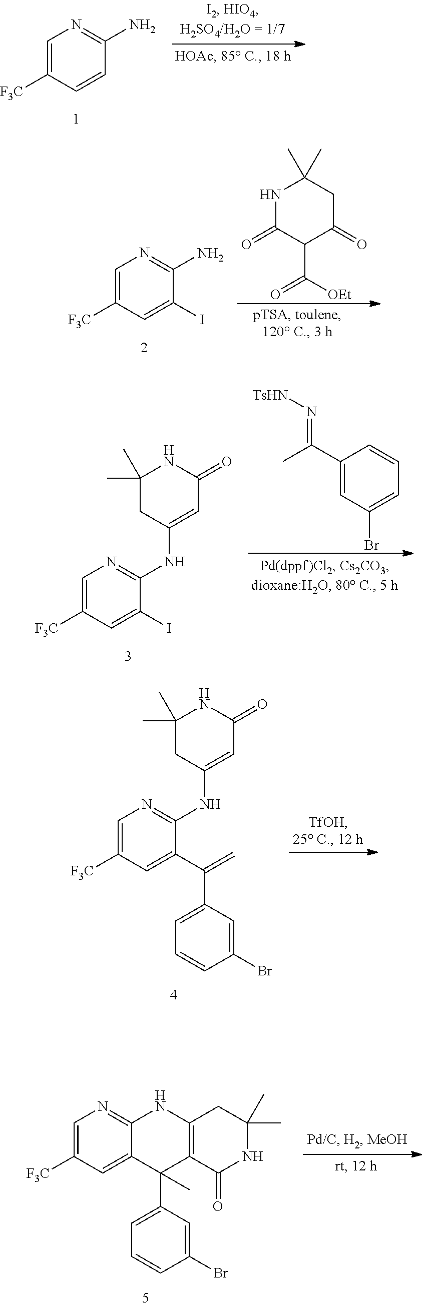

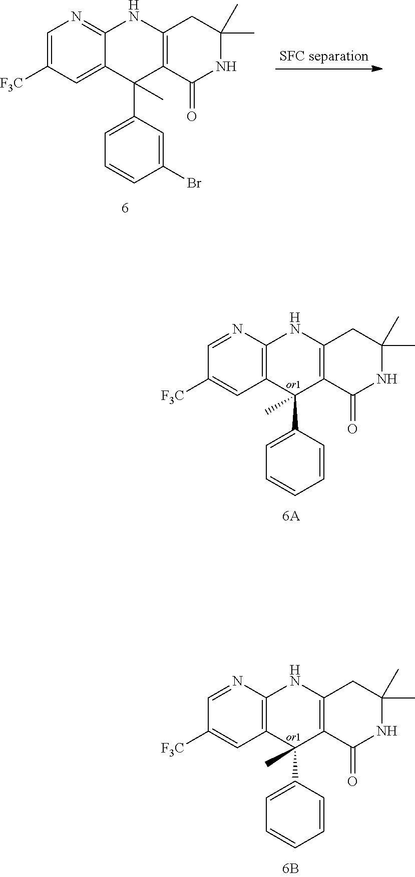 [SMILES: CC1(C)CC2=C(C(=O)N1)C(C)(c1cccc(Br)c1)c1cc(C(F)(F)F)cnc1N2 ; CC1(C)CC2=C(C(=O)N1)[C@@](C)(c1ccccc1)c1cc(C(F)(F)F)cnc1N2 ; CC1(C)CC2=C(C(=O)N1)[C@](C)(c1ccccc1)c1cc(C(F)(F)F)cnc1N2]

### Step 1: 3-iodo-5-(trifluoromethyl)pyridin-2-amine (2)

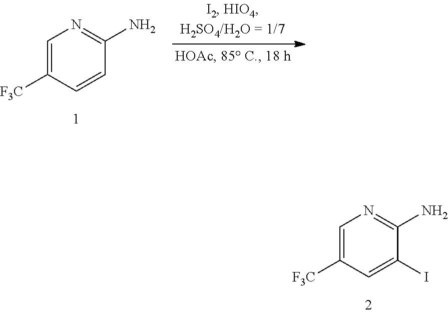 [SMILES: Nc1ccc(C(F)(F)F)cn1 ; Nc1ncc(C(F)(F)F)cc1I]

To a solution of 5-(trifluoromethyl)pyridin-2-amine (10.0) g, 61.7 mmol, 1.00 eq) and 12 (6.26 g, 24.67 mmol, 0.4 eq) in HOAc (100 mL) and H₂SO4:H₂O=1/7 (8 mL) was added HIO₄ (2.37 g, 12.34 mmol, 0.2 eq) portion wise with the temperature was kept at 25° C. under N₂ to get a red solution. The mixture was stirred at 85° C. for 18 h. On completion, the mixture quenched by 100 ml H₂O and basified to pH=7 with NaOH (2M). The reaction mixture was diluted with EtOAc (50 mL). The organic layer was washed with brine (200 mL), dried over Na₂SO₄, filtered, and concentrated in vacuo. The residue was purified by column chromatography (SiO₂, Petroleum ether/Ethyl acetate=5/1) to give compound 2 (19.4 g, 98% yield) as a yellow solid.

¹H NMR (400 MHz, DMSO-d₆) δ=8.26 (br s, 1H), 8.15 (br s, 1H), 6.86 (br s, 2H), 1.12 (s, 1H).

### Step 2: 4-((3-iodo-5-(trifluoromethyl)pyridin-2-yl)amino)-6,6-dimethyl-5,6-dihydropyridin-2(1H)-one (3)

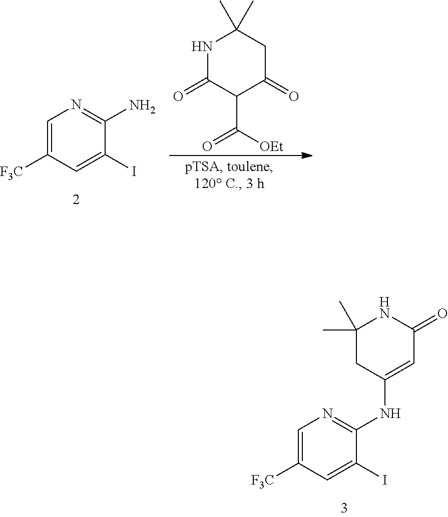

To a solution of compound 2 (3.00 g, 10.4 mmol, 1.00 eq) and ethyl 6,6-dimethyl-2,4-dioxo-piperidine-3-carboxylate (2.22 g, 10.4 mmol, 1.00 eq) in Toluene (30 mL) was added pTSA (199 mg, 1.04 mmol, 0.100 eq), the mixture was stirred at 120° C. for 3 h. On completion, the reaction mixture was poured into saturated NH₄Cl (50 mL) and extracted with EtOAc (50 mL×3). The combined organic layer was washed with brine (50 mL×3), dried over Na₂SO₄, filtered, and concentrated in vacuo. The residue was purified by column chromatography (SiO₂, Petroleum ether/Ethyl acetate=3/1) to give compound 3 (2 g, 42% yield) as a yellow solid.

LCMS: tR=0.428 min., (ES⁺) m/z (M+H)⁺=411.9.

### Step 3: 4-(3-(1-(3-bromophenyl)vinyl)-5-(trifluoromethyl)pyridin-2-yl)amino)-6,6-dimethyl-5,6-dihydropyridin-2(1H)-one (4)

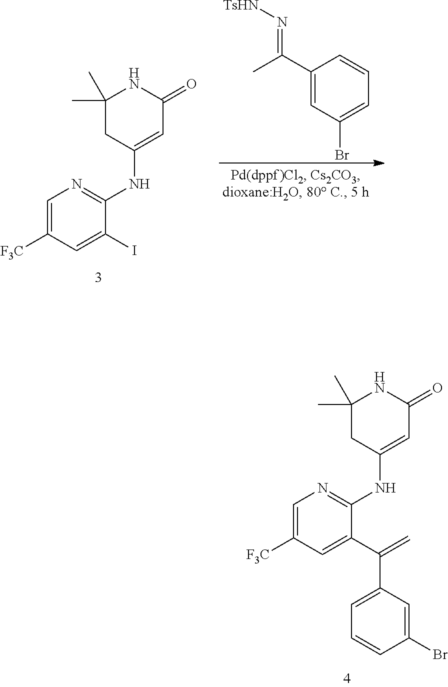

To a solution of compound 3 (1.98 g, 4.82 mmol, 1.10 eq) and N-[(E)-1-(3-bromophenyl)ethylideneamino]-4-methyl-benzenesulfonamide (1.61 g, 4.38 mmol, 1.00 eq) in dioxane (16 mL) and H₂O (5 mL) was added CsCO₃ (4.28 g, 13.1 mmol, 2.99 eq) portion wise with the temperature was kept at 25° C. under N₂ to get a yellow solution. The mixture was stirred at 80° C. for 5 h. On completion, the reaction mixture was poured into saturated NH₄Cl aqueous solution (50 mL) and extracted with EtOAc (100 mL×3). The combined organic layer was washed with brine (50 mL), dried over Na₂SO₄, filtered, and concentrated in vacuo. The crude product was purified by re-MPLC to give compound 4 (120 mg, 4% yield) as a yellow solid.

¹HNMR (400 MHz, DMSO-de) δ=8.72 (d, J=1.2 Hz, 1H), 8.16 (s, 1H), 7.93 (d, J=2.4 Hz, 1H), 7.83 (s, 1H), 7.54-7.50 (m, 4H), 6.08 (s, 1H), 6.05 (s, 1H), 5.67 (s, 1H), 1.99 (s, 2H), 1.05 (s, 6H).

### Step 4: 5-(3-bromophenyl)-5,8,8-trimethyl-3-(trifluoromethyl)-5,8,9,10-tetrahydropyrido[2,3-b][1,6]naphthyridin-6(7H)-one (5)

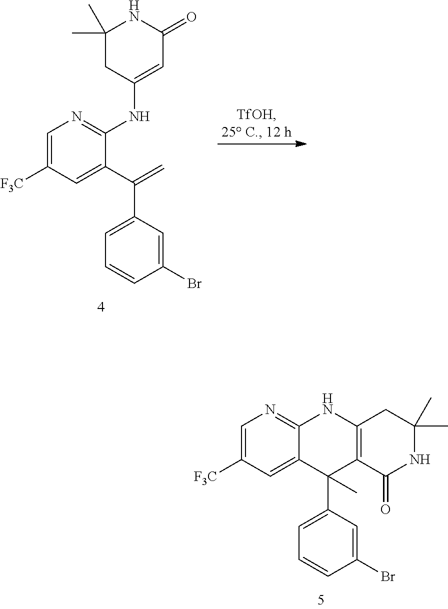 [SMILES: C=C(c1cccc(Br)c1)c1cc(C(F)(F)F)cnc1NC1=CC(=O)NC(C)(C)C1 ; CC1(C)CC2=C(C(=O)N1)C(C)(c1cccc(Br)c1)c1cc(C(F)(F)F)cnc1N2]

To a solution of compound 4 (100 mg, 0.214 mmol, 1.00 eq) in TfOH (2 mL) was stirred at 25° C. for 12 h. On completion, the reaction mixture was poured into NH₃·H₂O aqueous solution (5 mL) and extracted with EtOAc (5 mL×3). The combined organic layer was washed with brine (10 mL), dried over Na₂SO₄, filtered, and concentrated in vacuo. The residue was purified by column chromatography (SiO₂, Petroleum ether/Ethyl acetate=3/1) to give compound 5 (30 mg, 27% yield) as a yellow solid.

¹HNMR (400 MHz, DMSO-d₆) δ=10.00 (s, 1H), 8.33 (s, 1H), 7.53 (s, 1H), 7.40-7.29 (m, 3H), 7.27-7.22 (m, 1H), 6.70 (s, 1H), 1.98 (s, 3H), 1.22 (s, 6H).

### Step 5: (S)-5,8,8-trimethyl-5-phenyl-3-(trifluoromethyl)-5,8,9,10-tetrahydropyrido[2,3-b][1,6] naphthyridin-6(7H)-one (6A)

### (R)-5,8,8-trimethyl-5-phenyl-3-(trifluoromethyl)-5,8,9,10-tetrahydropyrido[2,3-b][1,6]naphthyridin-6(7H)-one (6B)

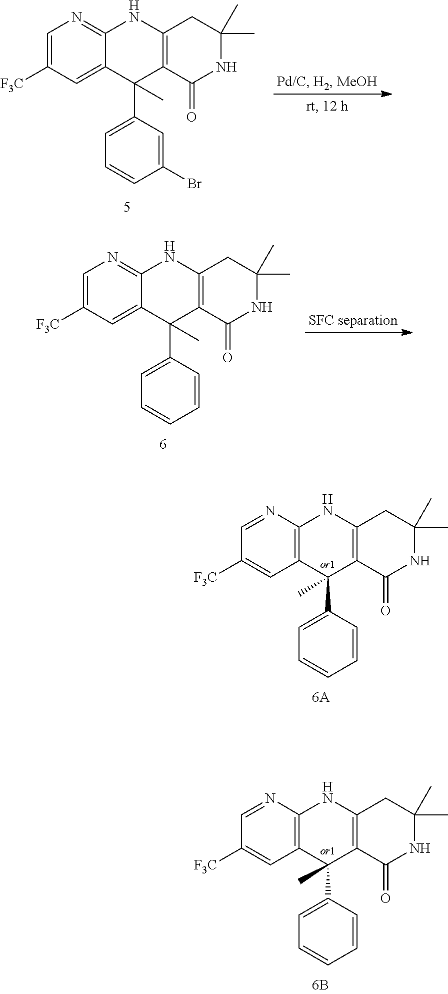 [SMILES: CC1(C)CC2=C(C(=O)N1)C(C)(c1cccc(Br)c1)c1cc(C(F)(F)F)cnc1N2 ; CC1(C)CC2=C(C(=O)N1)C(C)(c1ccccc1)c1cc(C(F)(F)F)cnc1N2 ; CC1(C)CC2=C(C(=O)N1)[C@@](C)(c1ccccc1)c1cc(C(F)(F)F)cnc1N2 ; CC1(C)CC2=C(C(=O)N1)[C@](C)(c1ccccc1)c1cc(C(F)(F)F)cnc1N2]

To a solution of compound 5 (40 mg, 0.0643 mmol, 1.00 eq) and Pd/C (6.8 mg, 0.0643 mmol, 1.00 eq) in MeOH (0.5 mL) was stirred at 25° C. for 12 h under H₂. On completion, the mixture was filtered and concentrated in vacuo to give the residue. The residue was purified by prep-HPLC (column: Welch Xtimate C18 150×25 mm×5 um; mobile phase: [water (0.05% FA)-ACN]; B %; 20%-80%, 15 min) to give to give 6 (30 mg, 90% yield) as a white solid.

The product 30 mg was separated by SFC (Chiralcel OD-3 50×4.6 mm I.D., 3 um, Mobile phase: MeOH (0.05% DEA) in CO₂ from 5% to 40%; Flow rate: 3 mL/min Wavelength: 220 nm to) to give:

Peak 1: (S)-5,8,8-trimethyl-5-phenyl-3-(trifluoromethyl)-5,8,9,10-tetrahydropyrido[2,3-b][1,6] naphthyridin-6(7H)-one (6A). (3.7 mg, 12% yield).

LCMS: tR=0.437 min., (ES⁺) m/z (M+H)⁺=388.

¹H NMR (400 MHz, DMSO-d₆) δ=10.02 (s, 1H), 8.02 (d, J=2.8 Hz, 1H), 7.87 (t, J=1.6 Hz, 1H), 7.68 (td, J=1.6, 8.0 Hz, 2H), 7.55-7.50 (m, 1H), 7.10 (dd, J=2.8, 9.2 Hz, 1H), 3.18 (s, 3H), 2.48-2.42 (m, 2H), 2.07-2.02 (m, 1H), 1.98-1.91 (m, 4H), 1.01 (d, J=7.2 Hz, 6H).

Peak 2: (R)-5,8,8-trimethyl-5-phenyl-3-(trifluoromethyl)-5,8,9,10-tetrahydropyrido[2,3-b][1,6]naphthyridin-6(7H)-one (6B). (13.7 mg, 45% yield).

LCMS: tR=0.459 min., (ES⁺) m/z (M+H)⁺=388.

¹H NMR (400 MHz, DMSO-d₆) δ=9.92 (s, 1H), 8.30 (s, 1H), 7.37 (d, J=7.6 Hz, 2H), 7.30-7.22 (m, 3H), 7.14-7.07 (m, 1H), 6.62 (s, 1H), 4.13-4.04 (m, 4H), 3.18 (d, J=4.8 Hz, 11H), 2.00 (s, 3H), 1.25 (s, 2H), 1.22 (s, 6H).

### Example 1.54. (S)-5,8,8-trimethyl-5-(3-(methylsulfonyl)phenyl)-3-(trifluoromethyl)-5,8,9,10-tetrahydropyrido[2,3-b][1,6]naphthyridin-6(7H)-one (3A)

### (R)-5,8,8-trimethyl-5-(3-(methylsulfonyl)phenyl)-3-(trifluoromethyl)-5,8,9,10-tetrahydropyrido[2,3-b][1,6]naphthyridin-6(7H)-one (3B)

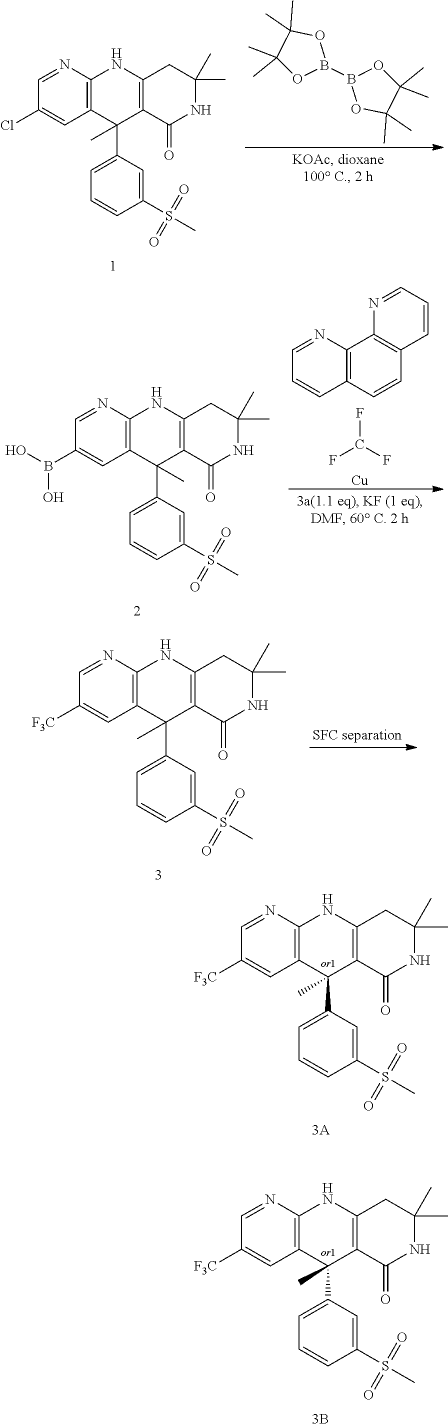

### Step 1: 5-bromo-4-fluoro-3-iodopyridin-2-amine (1)

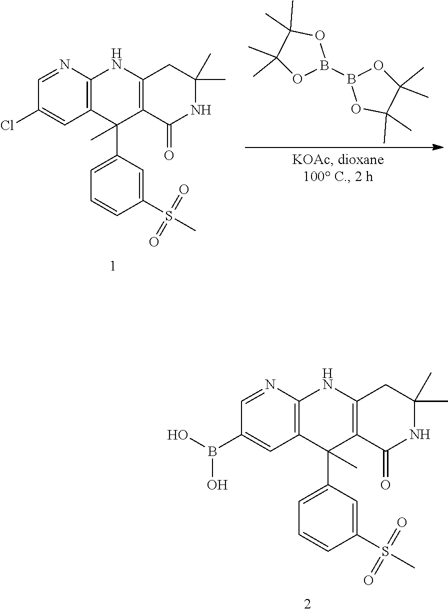

To a solution of compound 1 (800 mg, 1.85 mmol, 1.00 eq) and 4,4,5,5-tetramethyl-2-(4,4,5,5-tetramethyl-1,3,2-dioxaborolan-2-yl)-1,3,2-dioxaborolane (941 mg, 3.70 mmol, 2.00 eq) in dioxane (8 mL) was added KOAc (908 mg, 9.26 mmol, 5.00 eq) and XPhos Pd G3 (157 mg, 0.185 mmol, 0.100 eq) portion wise with the temperature was kept at 25° C. under N₂ to get a yellow solution. The reaction mixture was stirred at 100° C. for 2 hours. On completion, the reaction mixture was quenched by addition of saturated NH₄Cl (5 mL), and then extracted with EtOAc (5 mL×3). The combined organic layers were washed with brine (5 mL×3), dried over Na₂SO₄, filtered, and concentrated under reduced pressure to give a residue. The residue was purified by flash silica gel chromatography (ISCO®; 10 g SepaFlash® Silica Flash Column, Eluent of 0˜100% Ethyl acetate/Petroleum ether gradient @ 60 mL/min) to give compound 1 (700 mg, crude) as a white solid.

LCMS: tR=0.266 min., (ES⁺) m/z (M+H)⁺=442.1.

### Step 3: (S)-5,8,8-trimethyl-5-(3-(methylsulfonyl)phenyl)-3-(trifluoromethyl)-5,8,9,10-tetrahydropyrido[2,3-b][1,6]naphthyridin-6(7H)-one (3A)

### (R)-5,8,8-trimethyl-5-(3-(methylsulfonyl)phenyl)-3-(trifluoromethyl)-5,8,9,10-tetrahydropyrido[2,3-b][1,6]naphthyridin-6(7H)-one (3B)

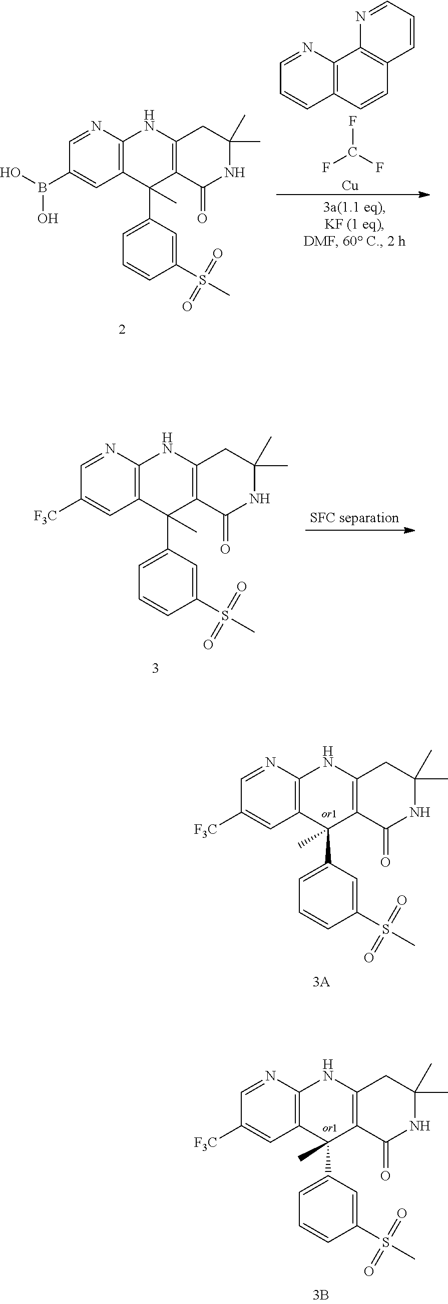

To a solution of compound 1 (370 mg, 0.838 mmol, 1.00 eq) and trifluoromethyl(1,10-phenanthroline)copper (288 mg, 0.922 mmol, 1.10 eq) in DMF (0.5 mL) was added KF (49 mg, 0.838 mmol, 1.00 eq) portion wise with the temperature was kept at 25° C. under N₂ to get a yellow solution. The reaction mixture was stirred at 60° C. for 2 hours. On completion, the reaction mixture was quenched by addition of saturated NH₄Cl (5 mL), and then extracted with EtOAc (5 mL×3). The combined organic layers were washed with brine (5 mL×3), dried over Na₂SO₄, filtered, and concentrated under reduced pressure to give a residue. The residue was purified by re-MPLC (0.1% FA, 5˜75% ACN/H2O) to give compound 2 (45 mg, 12% yield) as a white solid.

¹H NMR (400 MHz, DMSO-d₆) δ=10.06 (s, 1H), 8.34 (d, J=1.2 Hz, 1H), 7.91 (s, 1H), 7.71 (dd, J=8.0, 16.8 Hz, 2H), 7.58-7.53 (m, 1H), 7.35 (d, J=2.0 Hz, 1H), 6.72 (s, 1H), 3.16 (s, 3H), 2.58-2.53 (m, 1H), 2.47 (s, 1H), 2.05 (s, 3H), 1.21 (s, 6H).

LCMS: tR=0.385 min., (ES⁺) m/z (M+H)⁺=466.2.

The product was separated by SFC (column: (S, S) Chiralpak AD-3 50×4.6 mm; mobile phase: [0.05% DEA IPA]; B %; 5%-40%) to give:

Peak 1: (S)-5,8,8-trimethyl-5-(3-(methylsulfonyl)phenyl)-3-(trifluoromethyl)-5,8,9,10-tetrahydropyrido[2,3-b][1,6]naphthyridin-6(7H)-one (3A) (16 mg, 0.0345 mmol, 32% yield).

LCMS: tR=0.409 min., (ES⁺) m/z (M+H)⁺=465.9.

¹H NMR (400 MHz, DMSO-d₆) δ=10.06 (br s, 1H), 8.34 (s, 1H), 7.91 (s, 1H), 7.71 (dd, J=8.0, 16.8 Hz, 2H), 7.59-7.52 (m, 1H), 7.35 (d, J=1.6 Hz, 1H), 6.71 (s, 1H), 3.16 (s, 3H), 2.58-2.53 (m, 1H), 2.47 (s, 1H), 2.05 (s, 3H), 1.21 (s, 6H).

Peak 2: (R)-5,8,8-trimethyl-5-(3-(methylsulfonyl)phenyl)-3-(trifluoromethyl)-5,8,9,10-tetrahydropyrido[2,3-b][1,6]naphthyridin-6(7H)-one (3B) (22 mg, 0.0453 mmol, 42% yield).

LCMS: tR=0.419 min., (ES⁺) m/z (M+H)⁺=466.1.

¹H NMR (400 MHz, DMSO-d₆) δ=10.06 (s, 1H), 8.34 (d, J=1.2 Hz, 1H), 7.91 (s, 1H), 7.71 (dd, J=8.0, 16.8 Hz, 2H), 7.58-7.53 (m, 1H), 7.35 (d, J=2.0 Hz, 1H), 6.72 (s, 1H), 3.16 (s, 3H), 2.58-2.53 (m, 1H), 2.47 (s, 1H), 2.05 (s, 3H), 1.21 (s, 6H).

### Example 1.55. (S)-5,88-trimethyl-5-(3-(methylsulfonyl)phenyl)-7,8,9,10-tetrahydropyrido[2,3-b][1,6]naphthyridin-6(5H)-one (3A)

### (R)-5,8,8-trimethyl-5-(3-(methylsulfonyl)phenyl)-7,8,9,10-tetrahydropyrido[2,3-b][1,6]naphthyridin-6(5H)-one (3B)

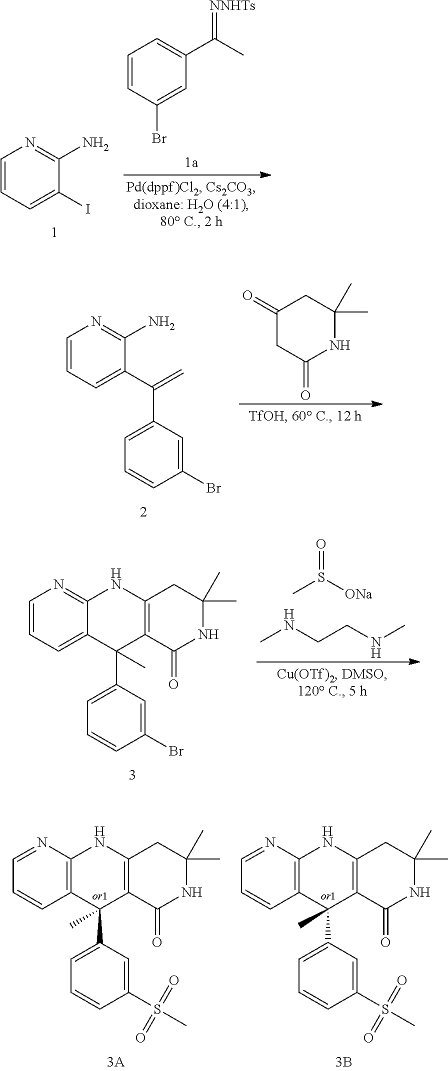

### Step 1: 3-(1-(3-bromophenyl)vinyl)pyridin-2-amine (2)

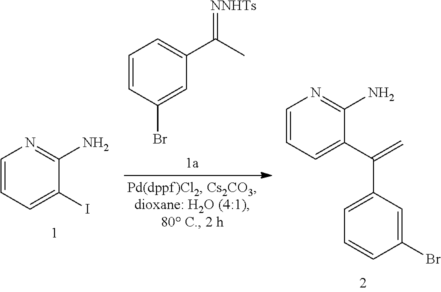

To a solution of compound 1 (5.00 g, 22.7 mmol, 1.00 eq) and 1a (9.18 g, 25.0 mmol, 1.10 eq) in 1,4-dioxane (40 mL) and H₂O (10 mL) was added Cs₂CO₃ (22.18 g, 68.2 mmol, 3.00 eq) and Pd(dppf)Cl₂CH₂Cl₂ (1.86 g, 2.27 mmol, 0.10 eq) in one portion stirred under N₂ at 80° C. for 2 h. On completion, the reaction was diluted with water (50 mL), extracted with EtOAc (3×50 mL), washed with saturated NaCl (90 mL), dried over Na₂SO₄, filtered, and concentrated under reduced pressure to give a residue. The residue was purified by flash silica gel chromatography (ISCO®; 80 g SepaFlash® Silica Flash Column, Eluent of 28˜35% Ethyl acetate/Petroleum ether gradient (4) 80 mL/min) to give compound 2 (5.80 g, 93% yield) as a yellow solid.

¹H NMR (400 MHz, DMSO-d₆) δ=7.98 (dd, J=1.6, 4.8 Hz, 1H), 7.51 (br d, J=7.2 Hz, 1H), 7.45 (s, 1H), 7.33-7.25 (m, 3H), 6.62 (dd, J=4.8, 7.2 Hz, 1H), 5.90 (s, 1H), 5.37 (s, 3H).

### Step 2: 5-(3-bromophenyl)-5,8,8-trimethyl-7,8,9,10-tetrahydropyrido[2,3-b][1,6]naphthyridin-6(5H)-one (3)

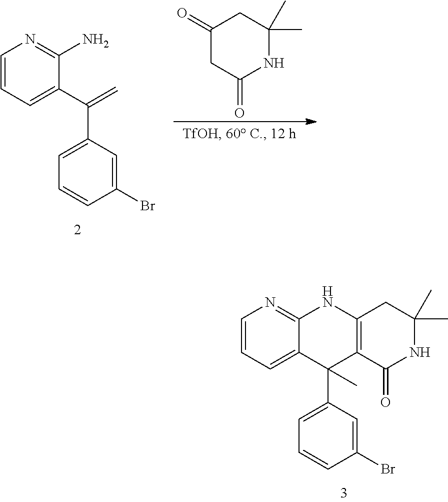

To a solution of compound 2 (5.80 g, 21.1 mmol, 1.00 eq) in TfOH (58 mL) added 6,6-dimethylpiperidine-2,4-dione (3.57 g, 25.3 mmol, 1.20 eq) was stirred at 60 C for 12 h. On completion, the reaction was basified with NaOH to pH=9, the residue was diluted with water (100 mL), extracted with EtOAc (3×100 mL), washed with saturated NaCl (150 mL), dried over Na₂SO₄, filtered, and concentrated under reduced pressure to give a residue. The residue was triturated from (Petroleum ether:Ethyl acetate=10:1, 30 mL) at 20° C. to give compound 3 (2.40 g) as a yellow solid.

¹H NMR (400 MHz, DMSO-d6) δ=9.48 (br s, 1H), 7.93 (br d, J=3.2 Hz, 1H), 7.45 (br s, 1H), 7.34 (br d, J=7.2 Hz, 1H), 7.22 (td, J=7.2, 14.8 Hz, 2H), 7.07 (br d, J=7.2 Hz, 1H), 6.82-6.69 (m, 1H), 6.53 (br s, 1H), 2.49-2.46 (m, 2H), 1.91 (br s, 3H), 1.19 (br s, 6H).

### Step 3: (S)-5,8,8-trimethyl-5-(3-(methylsulfonyl)phenyl)-7,8,9,10-tetrahydropyrido[2,3-b][1,6]naphthyridin-6(5H)-one (3A)

### (R)-5,8,8-trimethyl-5-(3-(methylsulfonyl)phenyl)-7,8,9,10-tetrahydropyrido[2,3-b][1,6]naphthyridin-6(5H)-one (3B)

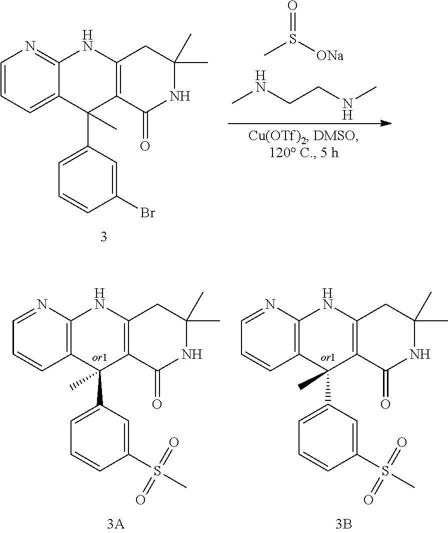

To a solution of sodium methanesulfinate (205 mg, 2.01 mmol, 2.00 eq) in DMSO (4 mL) was added Cu(OTf)₂ (362 mg, 1.00 mmol, 1.00 eq) and N1,N2-dimethylethane-1,2-diamine (186 mg, 2.11 mmol, 2.10 eq) in one portion stirred under N₂ at 20° C. for 0.08 h. Then a solution of compound 3 (400 mg, 1.00 mmol, 1.00 eq) in DMSO (4 mL) was added dropwise to the mixture, the mixture was stirred under N₂ at 120° C. for 3 h. On completion, the reaction was diluted with water (10 mL), extracted with EtOAc (3×10 mL), washed with NH₃·H₂O (30 mL) washed with saturated NaCl (15 mL), dried over Na₂SO₄, filtered, and concentrated under reduced pressure to give a residue. The crude product was purified by reversed phase (Column: Phenomenex Gemini 150×25 mm×10 um, water (0.1% NH₃·H₂O)-ACN, 35-45% ACN, 10 min).

The product was separated by SFC (AD-3-IPA(DEA)-5-40-3 mL-35T.l cm; Column: Chiralpak AD-3 50×4.6 mm I.D, 3 um, Mobile phase: Phase A for CO₂, and Phase B for IPA (0.05% DEA); Gradient elution: B in A from 5% to 40%, Flow rate: 3 mL/min; Detector: PDA; Column Temp: 35 C; Back Pressure: 100 Bar) to give:

Peak 1: (S)-5,8,8-trimethyl-5-(3-(methylsulfonyl)phenyl)-7,8,9,10-tetrahydropyrido[2,3-b][1,6]naphthyridin-6(5H)-one (3A) (29 mg, 7% yield).

LCMS: tR=0.290 min., (ES⁺) m/z (M+H)⁺=398.0.

¹H NMR (400 MHz, DMSO-d6) δ=9.52 (s, 1H), 7.95 (dd, J=1.6, 4.8 Hz, 1H), 7.87 (s, 1H), 7.68 (dd, J=8.0, 16.4 Hz, 2H), 7.55-7.50 (m, 1H), 7.06 (dd, J=1.2, 7.6 Hz, 1H), 6.75 (dd, J=4.8, 7.6 Hz, 1H), 6.53 (s, 1H), 3.15 (s, 3H), 2.48 (br s, 2H), 1.99 (s, 3H), 1.20 (s, 6H).

Peak 2: (R)-5,8,8-trimethyl-5-(3-(methylsulfonyl)phenyl)-7,8,9,10-tetrahydropyrido[2,3-b][1,6]naphthyridin-6(5H)-one (3B). (24 mg, 6% yield).

LCMS: tR=0.295 min., (ES⁺) m/z (M+H)⁺=398.0.

¹H NMR (400 MHz, DMSO-d6) δ=9.52 (s, 11H), 7.95 (dd, J=1.6, 4.8 Hz, 1H), 7.87 (t, J=1.6 Hz, 1H), 7.68 (dd, J=8.0, 16.8 Hz, 2H), 7.55-7.50 (m, 1H), 7.06 (dd, J=1.2, 7.6 Hz, 1H), 6.75 (dd, J=4.8, 7.6 Hz, 1H), 6.53 (s, 1H), 3.15 (s, 3H), 2.48 (br s, 2H), 1.99 (s, 3H), 1.20 (s, 6H).

### Example 1.56. (S)-3-fluoro-5,8,8-trimethyl-5-(3-(methylsulfonyl)phenyl)-7,8,9,10-tetrahydropyrido[2,3-b][1,6]naphthyridin-6(5H)-one (4A)

### (R)-3-fluoro-5,8,8-trimethyl-5-(3-(methylsulfonyl)phenyl)-7,8,9,10-tetrahydropyrido[2,3-b][1,6]naphthyridin-6(5H)-one (4B)

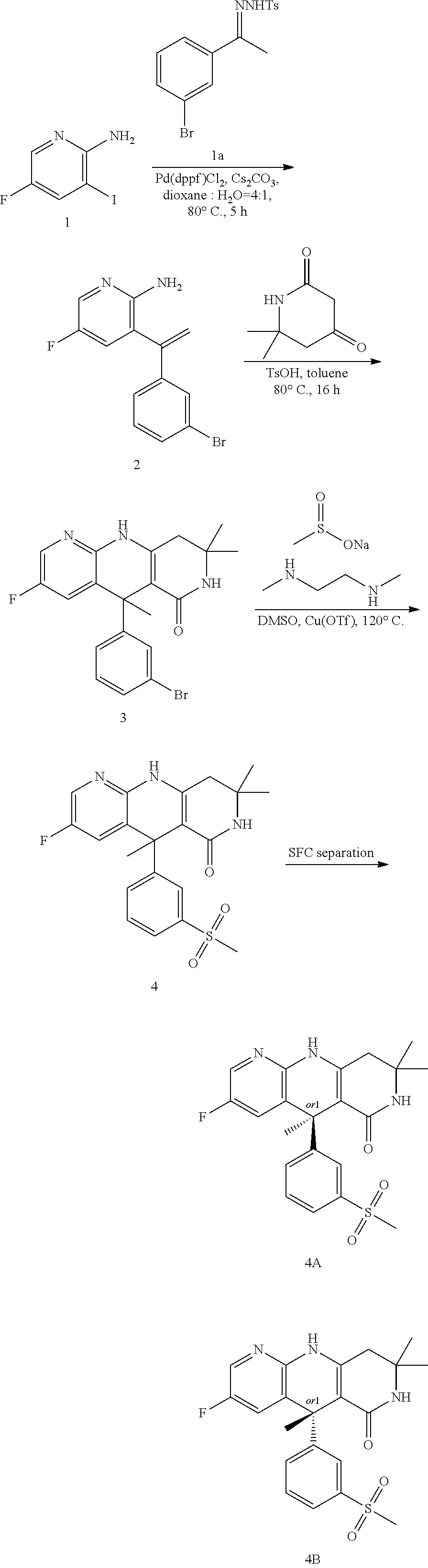

### Step 1: 3-(1-(3-bromophenyl)vinyl)-5-fluoropyridin-2-amine (2)

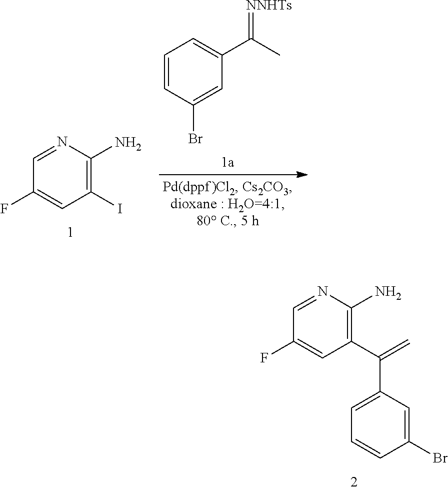

To a suspension of compound 1 (5.00 g, 21.0 mmol, 1 eq), compound 1a (9.26 g, 25.2 mmol, 1.2 eq) and Cs₂CO₃ (20.54 g, 63.0 mmol, 3 eq) in H₂O (10 mL) and dioxane (40 ML) was added Pd(dppf)Cl₂ (1.52 g, 2.10 mmol, 0.1 eq) portion wise under N₂ to get a yellow solution. The reaction mixture was stirred at 80° C. for 5 h. On completion, the reaction mixture was poured into saturated NH₄Cl aqueous solution (100 mL) and extracted with EtOAc (250 mL×3). The combined organic layer was washed with brine (100 mL), dried over Na₂SO₄, filtered, and concentrated. The residue was purified by flash silica gel chromatography (ISCO®; 120 g SepaFlash® Silica Flash Column, Eluent of 0˜15% Ethyl acetate/Petroleum ether gradient (100 mL/min) to give compound 2 (3.83 g, 60% yield) as a yellow solid.

LCMS: tR=0.370 min., (ES⁺) m/z (M+H)⁺=295.0.

### Step 2: 5-(3-bromophenyl)-3-fluoro-5,8,8-trimethyl-7,8,9,10-tetrahydropyrido[2,3-b][1,6]naphthyridin-6(5H)-one (3)

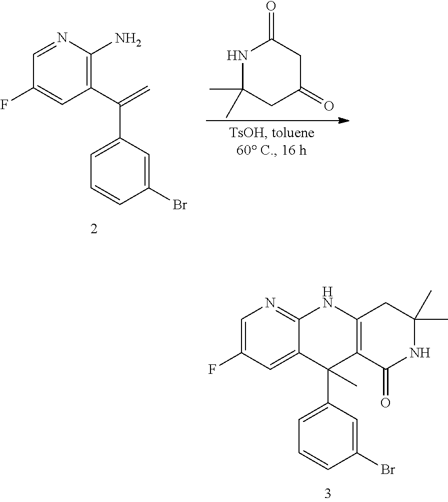

To a suspension of compound 2 (1.90 g, 6.48 mmol, 1 eq) in TfOH (10 mL) was added 6,6-dimethylpiperidine-2,4-dione (1.09 mg, 7.78 mmol, 1.20 eq). The reaction mixture was stirred at 60° C. for 16 h. On completion, the reaction mixture was adjusted to pH=8 saturated Na₂CO₃ aqueous solution and extracted with EtOAc (150 mL×3). The combined organic layer was washed with brine (50 mL), dried over Na₂SO₄, filtered, and concentrated. The crude product was triturated with PE:EA=5:1 at 25° C. for 30 min to give compound 3 (3.00 g, 60% yield) as brown solid.

LCMS: tR=0.458 min., (ES⁺) m/z (M+H)⁺=418.1.

¹H NMR (400 MHz, DMSO-d₆) δ=9.60 (br d, J=3.6 Hz, 1H), 7.96 (d, J=2.6 Hz, 1H), 7.49 (s, 1H), 7.35 (br d, J=7.6 Hz, 1H), 7.31-7.26 (m, 1H), 7.25-7.17 (m, 1H), 7.11-6.99 (m, 1H), 6.56 (s, 1H), 2.48 (br s, 2H), 1.64 (s, 3H), 1.20 (s, 6H).

### Step 3: (S)-3-fluoro-5,8,8-trimethyl-5-(3-(methylsulfonyl)phenyl)-7,8,9,10-tetrahydropyrido[2,3-b][1,6]naphthyridin-6(5H)-one (4A)

### (R)-3-fluoro-5,8,8-trimethyl-5-(3-(methylsulfonyl)phenyl)-7,8,9,10-tetrahydropyrido[2,3-b][1,6]naphthyridin-6(5H)-one (4B)

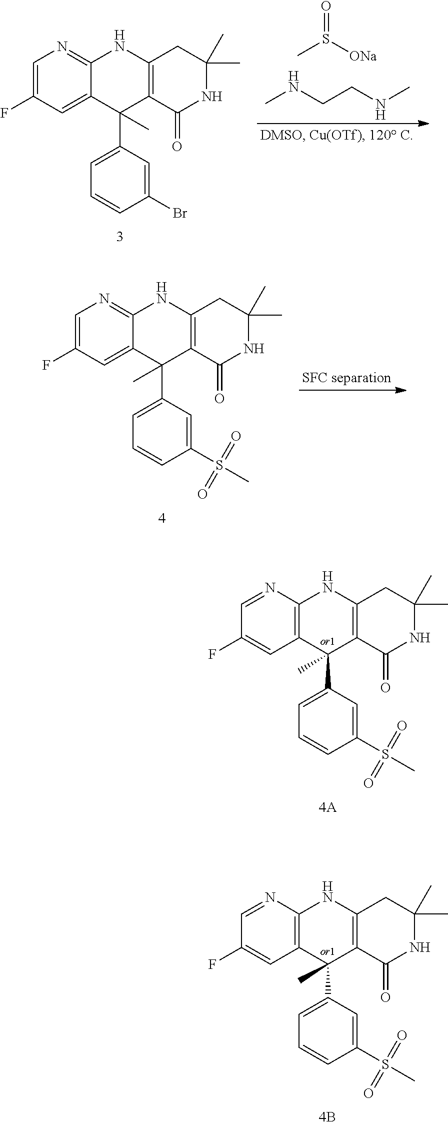

To a solution of sodium methanesulfinate (368 mg, 3.60 mmol, 2 eq) and Cu(OTf)₂ (650 mg, 1.80 mmol, 1.00 eq) in DMSO (15 mL) was added N¹,N²-dimethylethane-1,2-diamine (334 mg, 3.78 mmol, 2.10 eq) drop wise with the temperature was kept at 25° C. under N₂ to get a blue solution. The reaction mixture was stirred at 25° C. for 5 min. Then, the mixture was added compound 3 (1.50 g, 1.80 mmol, 1.00 eq) and was stirred at 120° C. for 12 h. On completion, the reaction mixture was poured into saturated NH₃·H₂O aqueous solution (50 mL) and extracted with EtOAc (100 mL×3). The residue was purified by re-MPLC (column: Welch Xtimate C18 150×25 mm×5 um; mobile phase: [water(0.05% NH₃·H₂O)-ACN]; B %: 15%-40%, 20 min) to give compound 4 (1 g, 66% yield).

The product 250 mg was separated by SFC (DAICEL CHIRALPAK AD-H (250 mm×30 mm, 5 um); Mobile phase: MeOH (0.1% NH₃—H₂O) in CO₂ from 25% to 25%; Flow rate: 120 mL/min Wavelength: 220 nm) to give:

Peak 1: (S)-3-fluoro-5,8,8-trimethyl-5-(3-(methylsulfonyl)phenyl)-7,8,9,10-tetrahydropyrido[2,3-b][1,6]naphthyridin-6(5H)-one (4A) (100 mg, 13% yield).

LCMS: tR=0.374 min., (ES⁺) m/z (M+H)⁺=416.1.

¹H NMR (400 MHz, DMSO-d₆) δ=9.63 (s, 1H), 7.98 (d, J=2.8 Hz, 1H), 7.88 (s, 1H), 7.72-7.63 (m, 2H), 7.59-7.49 (m, 1H), 7.13-7.03 (m, 1H), 6.56 (s, 1H), 3.17 (s, 3H), 2.56-2.51 (m, 1H), 2.49-2.42 (m, 1H), 2.03 (s, 3H), 1.20 (s, 6H).

Peak 2: (R)-3-fluoro-5,8,8-trimethyl-5-(3-(methylsulfonyl)phenyl)-7,8,9,10-tetrahydropyrido[2,3-b][1,6]naphthyridin-6(5H)-one (4B) (90 mg, 12% yield).

LCMS: tR=0.382 min., (ES⁺) m/z (M+H)⁺=416.1.

¹H NMR (400 MHz, DMSO-d₆) δ=9.63 (s, 1H), 7.98 (d, J=2.8 Hz, 1H), 7.88 (s, 1H), 7.69 (br t, J=9.2 Hz, 2H), 7.59-7.48 (m, 1H), 7.13-7.01 (m, 1H), 6.56 (s, 1H), 3.17 (s, 3H), 2.54 (br s, 1H), 2.49-2.42 (m, 1H), 2.03 (s, 3H), 1.20 (s, 6H).

### Example 1.57. (S)-3-cyclopropyl-5,8,8-trimethyl-5-(3-(methylsulfonyl)phenyl)-7,8,9,10-tetrahydropyrido [2,3-b][1,6]naphthyridin-6(5H)-one (5A)

### (R)-3-cyclopropyl-5,8,8-trimethyl-5-(3-(methylsulfonyl)phenyl)-7,8,9,10-tetrahydropyrido [2,3-b][1,6]naphthyridin-6(5H)-one (5B)

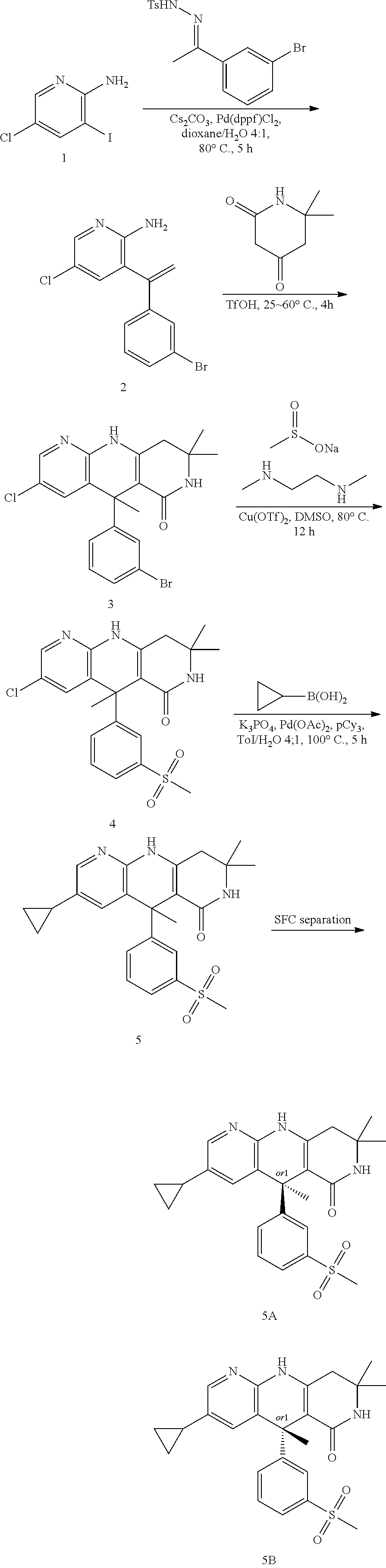

### Step 1: 3-(1-(3-bromophenyl)vinyl)-5-chloropyridin-2-amine (2)

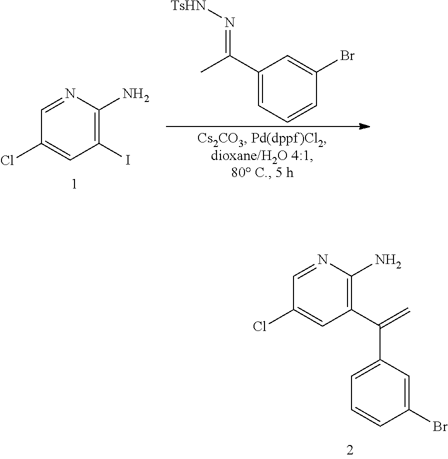

To a solution of compound 1 (5.00 g, 19.6 mmol, 1.00 eq) (E)-N′-(1-(3-bromophenyl) ethylidene)-4-methylbenzenesulfonohydrazide (866 mg, 23.6 mmol, 1.20 eq) and Cs₂CO₃ (1.9 g, 58.9 mmol, 3.00 eq) in dioxane (40 mL) and H₂O (10 mL) was added Pd(dppf)Cl₂ (1.4 g, 1.96 mmol, 0.10 eq) in one portion to the reaction mixture. The reaction mixture was stirred at 80° C. for 5 h. On completion, the reaction mixture was diluted with EtOAc (3×50 mL). The organic layer was washed with brine (2×50 mL), dried over Na₂SO₄, filtered, and concentrated.

The residue was purified by flash silica gel chromatography (ISCO®; 80 g SepaFlash® Silica Flash Column, Eluent of 0˜10% Ethyl acetate/Petroleum ether gradient @ 60 mL/min) to give compound 2 (4.70 g, 77% yield) as a yellow solid.

LCMS: tR=0.364 min., (ES⁺) m/z (M+H)⁺=311.2.

### Step 2: 5-(3-bromophenyl)-3-chloro-5,8,8-trimethyl-7,8,9,10-tetrahydropyrido[2,3-b][1,6]naphthyridin-6(5H)-one (3)

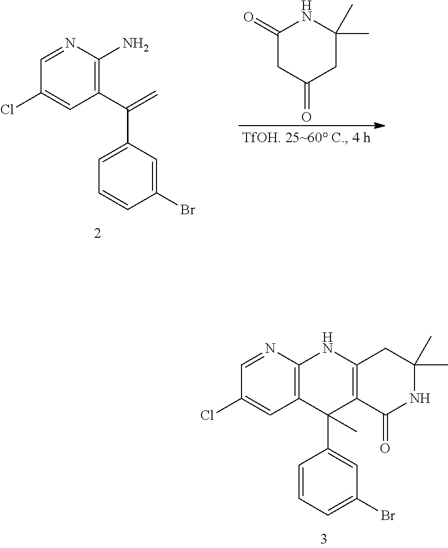

To a solution of compound 2 (500 mg, 1.62 mmol, 1.0 eq) in TfOH (5 mL) was added 6,6-dimethylpiperidine-2,4-dione (274 mg, 1.94 mmol, 1.20 eq) in one portion at 25° C. The reaction mixture was stirred at 60° C. for 4 h. On completion, the reaction mixture was adjusted to pH=7 Na₂CO₃ solution, The reaction mixture was diluted with EtOAc (3×20 mL). The organic layer was washed with brine (2×20 mL), dried over Na₂SO₄, filtered, and concentrated. The residue was purified by flash silica gel chromatography (ISCO®; 10 g SepaFlash® Silica Flash Column, Eluent of 0˜10% Ethyl acetate/Petroleum ether gradient @ 60 mL/min) to give compound 3 (458 mg, 66% yield) as a yellow solid.

LCMS: tR=0.457 min., (ES⁺) m/z (M+H)⁺=434.0.

### Step 3: 3-chloro-5,8,8-trimethyl-5-(3-(methylsulfonyl)phenyl)-7,8,9,10-tetrahydropyrido[2,3-b][1,6]naphthyridin-6(5H)-one (4)

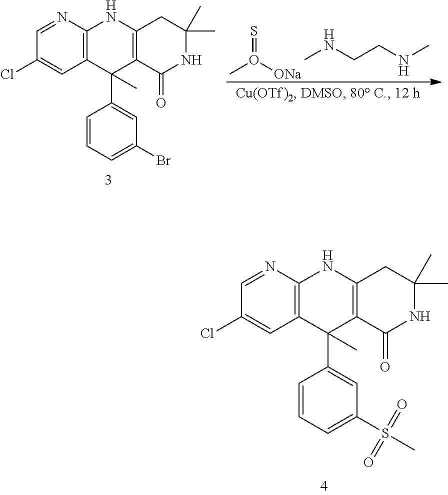

To a solution of sodium methanesulfinate (189 mg, 1.85 mmol, 2.00 eq) and N1,N2-dimethylethane-1,2-diamine (171 mg, 1.94 mmol, 2.10 eq) in DMSO (5 mL) was added Cu(OTf)₂ (334 mg, 0.92 mmol, 1.00 eq) in portion wise at 25° C. under N₂. Then compound 3 (400 mg, 0.92 mmol, 1.00 eq) was added to the reaction mixture and stirred at 80° C. for 12 h. On completion, the reaction mixture was adjusted to pH=8 with Na₂CO₃ solution. The reaction mixture was diluted with EtOAc (3×30 mL). The organic layer was washed with brine (3×30 mL), dried over Na₂SO₄, filtered, and concentrated. The residue was purified by flash silica gel chromatography (ISCO®; 10 g SepaFlash® Silica Flash Column, Eluent of 0˜10% Ethyl acetate/Petroleum ether gradient @ 60 mL/min) to give compound 4 (110 mg, 28% yield) as a yellow solid.

¹H NMR (400 MHz, DMSO-d₆) δ=9.74 (br s, 1H), 8.01 (br s, 1H), 7.89 (br s, 1H), 7.70 (br t, J=8.8 Hz, 2H), 7.60-7.51 (m, 1H), 7.17 (br s, 1H), 6.61 (br s, 1H), 3.18 (s, 3H), 2.55 (s, 2H), 2.07-1.90 (m, 4H), 1.20 (br s, 6H).

LCMS: tR=0.396 min., (ES+) m/z (M+H)⁺=432.0.

### Step 4: (S)-3-cyclopropyl-5,8,8-trimethyl-5-(3-(methylsulfonyl)phenyl)-7,8,9,10-tetrahydropyrido[2,3-b][1,6]naphthyridin-6(5H)-one (3A)

### (R)-3-cyclopropyl-5,8,8-trimethyl-5-(3-(methylsulfonyl)phenyl)-7,8,9,10-tetrahydropyrido[2,3-b][1,6]naphthyridin-6(5H)-one (3B)

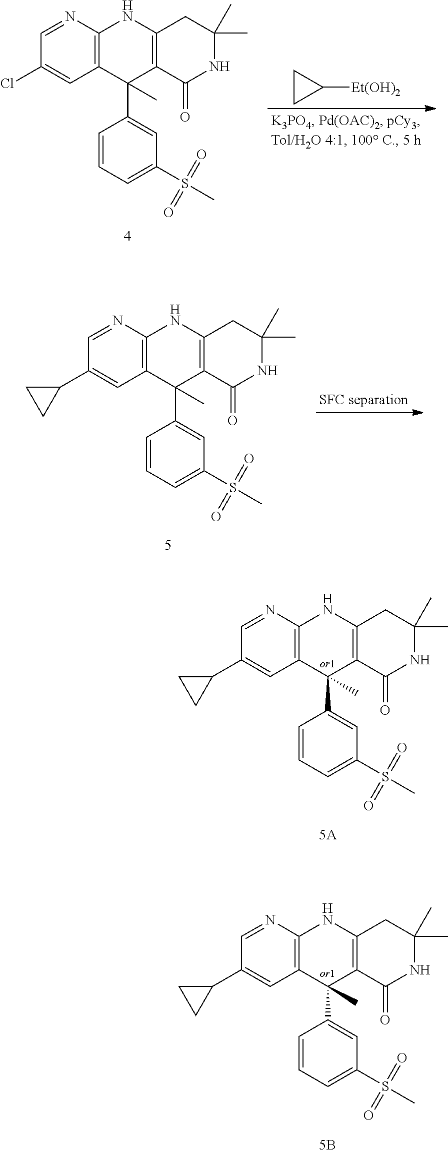

To a solution of compound 4 (104 mg, 0.24 mmol, 1.00 eq) cyclopropylboronic acid (25 mg, 0.28 mmol, 1.20 eq) PCy₃ (5.0 mg, 0.02 mmol, 0.10 eq) and K₃PO₄ (153 mg, 0.72 mmol, 3.00 eq) in dioxane (4 mL) and H₂O (1 mL) was added Pd(OAC)₂ (5.1 mg, 0.02 mmol, 0.10 eq) in one portion under N₂. The reaction mixture was stirred at 100° C. for 5 h. On completion. The reaction mixture was diluted with EtOAc (3×30 mL). The organic layer was washed with brine (2×30 mL), dried over Na₂SO₄, filtered, and concentrated. The residue was purified by flash silica gel chromatography (ISCO®; 10 g SepaFlash® Silica Flash Column. Eluent of 0˜10% Ethyl acetate/Petroleum ether gradient @ 60 mL/min) to give compound 5 (70 mg, 46% yield) as a yellow solid.

LCMS: tR=0.371 min., (ES⁺) m/z (M+H)⁺=438.0.

The yellow solid 70 mg was separated by SFC (Column: Chiralcel OD-3 50×4.6 mm I.D., 3 um Mobile phase: Phase A for CO₂, and Phase B for MeOH (0.05% DEA); Gradient elution: B in A from 5% to 40% Flow rate: 3 mL/min; Detector: PDA; Column Temp: 35 C; Back Pressure: 100 Bar) to give:

Peak 1: (S)-3-cyclopropyl-5,8,8-trimethyl-5-(3-(methylsulfonyl)phenyl)-7,8,9,10-tetrahydropyrido[2,3-b][1,6]naphthyridin-6(5H)-one (5A). (9.0 mg, 99.7% purity, 14% yield).

LCMS: tR=0.371 min., (ES⁺) m/z (M+H)⁺=438.0.

¹H NMR (400 MHz, DMSO-d₆) δ=9.42 (s, 1H), 7.89 (s, 11H), 7.72 (d, J=2.4 Hz, 1H), 7.67 (dd, J=7.8, 14.6 Hz, 2H), 7.55-7.49 (m, 1H), 6.77 (d, J=2.4 Hz, 1H), 6.48 (s, 1H), 3.16 (s, 3H), 2.46 (br s, 2H), 2.00 (s, 3H), 1.74-1.63 (m, 1H), 1.19 (s, 6H), 0.81-0.72 (m, 2H), 0.57-0.49 (m, 1H), 0.42-0.35 (m, 1H).

Peak 2: (R)-3-cyclopropyl-5,8,8-trimethyl-5-(3-(methylsulfonyl)phenyl)-7,8,9,10-tetrahydropyrido[2,3-b][1,6]naphthyridin-6(5H)-one (5B). (3.4 mg, 98% purity, 5% yield).

LCMS: tR=0.370 min., (ES⁺) m/z (M+H)⁺=438.0.

¹H NMR (400 MHz, DMSO-d₆) δ=9.42 (s, 1H), 7.89 (s, 11H), 7.72 (d, J=2.4 Hz, 1H), 7.67 (dd, J=7.8, 14.8 Hz, 2H), 7.55-7.49 (m, 1H), 7.27-7.23 (m, 1H), 7.14-7.09 (m, 1H), 6.99 (s, 1H), 6.77 (d, J=1.8 Hz, 1H), 6.48 (s, 1H), 3.16 (s, 3H), 2.46 (br s, 2H), 2.00 (s, 3H), 1.74-1.62 (m, 1H), 1.19 (s, 6H), 0.84-0.74 (m, 2H), 0.57-0.49 (m, 1H), 0.41-0.35 (m, 1H).

### Example 2. Synthesis of Additional Select Compounds

The compound numbers recited in each one of Examples 2.1 to 2.34 apply only to each one of Examples 2.1 to 2.34, respectively.

### Example 2.1. (R)-5-ethyl-8,8-dimethyl-5-phenyl-5,8,9,10-tetrahydrobenzo[b][1,8]naphthyridin-6(7H)-one. (4A)

### (S)-5-ethyl-8,8-dimethyl-5-phenyl-5,8,9,10-tetrahydrobenzo[b][1,8]naphthyridin-6(7H)-one. (4B)

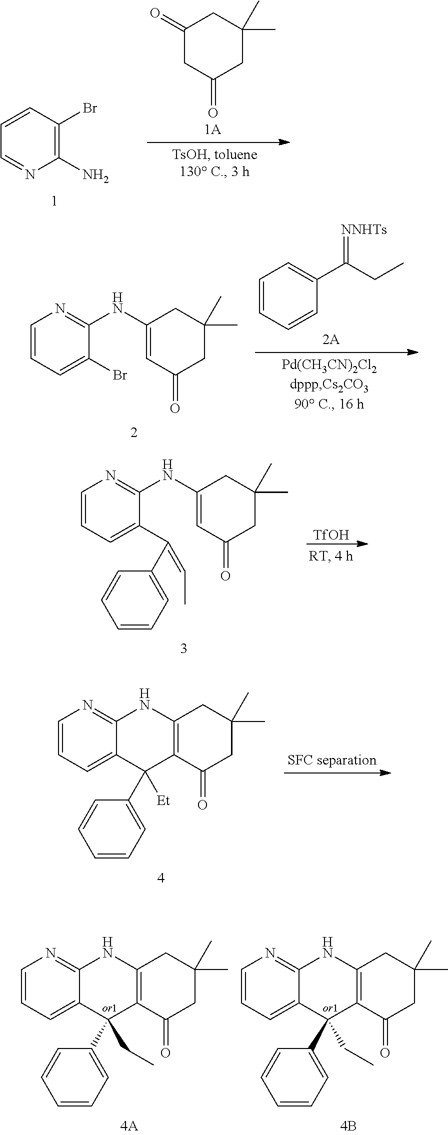

### Step 1: 3-((3-bromopyridin-2-yl)amino)-5,5-dimethylcyclohex-2-en-1-one. (2)

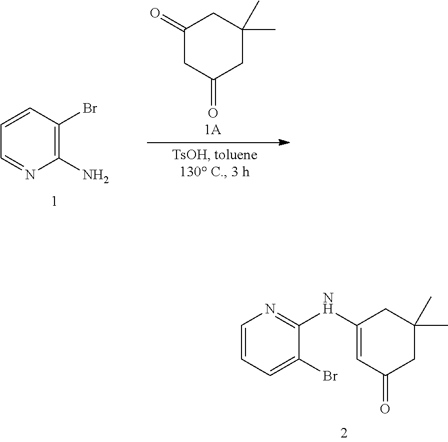

A solution of compound 1 (5.0 g, 28.9 mmol), compound 1A (12.0 g, 85.5 mmol), p-toluene sulfonic acid monohydrate (604.7 mg, 3.18 mmol) in toluene (150 mL) was stirred at 130° C. for 3 hours. TLC (PE/EtOAc=1/1) showed starting material remained and new spots were observed. The reaction solution was washed with saturated NaHCO₃ solution (100 mL×2), water (100 mL) and brine (100 mL), dried over Na₂SO₄ and filtered. The filtrate was concentrated, and the residue was purified by silica gel (from Petroleum ether/EtOAc=3/1 to EtOAc) to afford compound 2 (3.0 g, 35.17% yield) as a red oil.

LCMS: (ES⁺) M/Z (M+H)⁺: 295.0.

¹H NMR: (400 MHz, DMSO-d6) δ=8.39-8.28 (m, 2H), 8.09 (dd, J=1.6, 7.6 Hz, 1H), 7.05 (dd, J=4.8, 7.8 Hz, 1H), 6.19 (s, 1H), 2.08 (s, 2H), 1.02 (s, 6H).

### Step 2: (E)-5,5-dimethyl-3-((3-(1-phenylprop-1-en-1-yl)pyridin-2-yl)amino)cyclohex-2-en-1-one. (3)

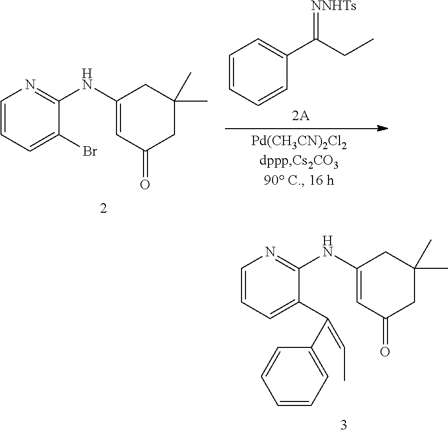

Under N₂, a mixture of compound 2 (600 mg, 2.03 mmol), compound 2A (613.8 mg, 2.03 mmol), Pd(CH3CN)₂Cl₂ (53 mg, 0.2 mmol), dppp (168 mg, 0.4 mmol), Cs₂CO₃ (1.32 g, 4.06 mmol) in dioxane (20 mL) was stirred at 90° C. for 16 hours. TLC (EtOAc) showed new spots were observed. The reaction suspension was diluted with water (100 mL) and extracted with EtOAc (100 mL×2). The combined organic layer was washed with water (50 mL) and brine (50 mL), dried over Na₂SO₄, and filtered. The filtrate was concentrated, and the residue was purified by silica gel (EtOAc) to afford a crude product. The crude product was further purified by prep-HPLC (Column: YMC-Actus Triart C18 150×30 mm×5 μm; Condition: water (0.225% FA)-ACN) to afford compound 3 (50 mg, 7.41% yield) as a yellow solid.

LCMS: (ES⁺) M/Z (M+H)⁺: 333.3.

¹H NMR: (400 MHz, methanol-d4) δ=8.42 (dd, J=2.0, 5.2 Hz, 1H), 7.72 (dd, J=1.2, 7.6 Hz, 1H), 7.31 (dd, J=5.2, 7.6 Hz, 1H), 7.28-7.16 (m, 3H), 7.16-7.11 (m, 2H), 6.43 (q, J=7.2 Hz, 1H), 6.08 (s, 1H), 2.18-2.05 (m, 4H), 1.74 (d, J=7.2 Hz, 3H), 0.89 (s, 6H).

### Step 3: (R)-5-ethyl-8,8-dimethyl-5-phenyl-5,8,9,10-tetrahydrobenzo[b][1,8]naphthyridin-6(7H)-one. (4A)

### (S)-5-ethyl-8,8-dimethyl-5-phenyl-5,8,9,10-tetrahydrobenzo[b][1,8]naphthyridin-6(7H)-one. (4B)

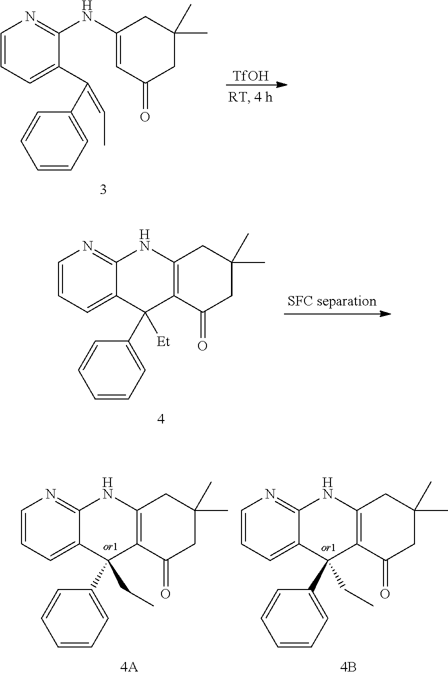

A solution of compound 3 (40.00 mg, 120.32 μmol) in TfOH (2.00 mL) was stirred at 20° C. for 4 hours. TLC (EtOAc) showed starting material was consumed and a new spot observed. The reaction solution was added into NaOH (4 N, 10 mL) and extracted with EtOAc (30 mL×2). The combined organic layer was washed with water (50 mL×2) and brine (50 mL), dried over Na₂SO₄, filtered. The filtrate was concentrated, and the residue was purified by prep-HPLC (YMC-Actus Triart C18 100×30 mm×5 μm, water (0.225% FA)-ACN) to afford 4 (15.00 mg, 37.50% yield) as a white solid.

LCMS: (ES⁺) M/Z (M+H)⁺: 333.0.

HPLC: (purity: 100%).

¹H NMR: (500 MHz, methanol-d₄) δ=8.07 (s, 1H), 7.94 (m, 1H), 7.38 (m, 2H), 7.21 (m, 2H), 7.12-7.03 (m, 2H), 6.80 (m, 7.7 Hz, 1H), 3.05-2.95 (m, 1H), 2.62-2.54 (m, 2H), 2.17-2.01 (m, 3H), 1.14 (s, 3H), 1.11 (s, 3H), 0.78 (t, J=14.5 Hz, 3H).

4 (15.00 mg) was separated by SFC (DAICEL CHIRALPAK AD (250 mm×50 mm, 10 μm); 0.1% NH₃H₂O EtOH; 35%; Flow Rate: 80 ml/min) to give:

(R)-5-ethyl-8,8-dimethyl-5-phenyl-5,8,9,10-tetrahydrobenzo[b][1,8]naphthyridin-6(7H)-one. (4A) (5.00 mg, 33.33% yield) as a white solid.

LCMS: tR=4.611 min., (ES⁺) M/Z (M+H)⁺=333.0.

HPLC: (purity: 100%).

¹H NMR (500 MHz, methanol-dt) δ=7.94 (m, 1H), 7.38 (m, 2H), 7.21 (m, 2H), 7.12-7.01 (m, 2H), 6.80 (m, 1H), 3.04-2.95 (m, 1H), 2.58 (s, 2H), 2.17-2.02 (m, 3H), 1.12 (d, J=14.0 Hz, 6H), 0.78 (t, J=14.5 Hz, 3H).

(S)-5-ethyl-8,8-dimethyl-5-phenyl-5,8,9,10-tetrahydrobenzo[b][1,8]naphthyridin-6(7H)-one. (4B) (5.00 mg, 33.33% yield) as a white solid.

LCMS: tR=5.393 min., (ES⁺) M/Z (M+H)⁺=333.0.

HPLC: (purity: 100%).

¹HNMR (500 MHz, methanol-d₄) δ=7.94 (m, 1H), 7.38 (m, 2H), 7.26-7.18 (m, 2H), 7.10-7.03 (m, 2H), 6.80 (m, 1H), 3.06-2.94 (m, 1H), 2.62-2.53 (m, 2H), 2.16-2.00 (m, 3H), 1.12 (d, J=14.0 Hz, 6H), 0.78 (t, J=14.5 Hz, 3H).

### Example 2.2. (5R,9R)-5-ethyl-9-fluoro-8,8-dimethyl-5-phenyl-5,8,9,10-tetrahydrobenzo[b][1,8]naphthyridin-6(7H)-one. (7A)

### (5R,9S)-5-ethyl-9-fluoro-8,8-dimethyl-5-phenyl-5,8,9,10-tetrahydrobenzo[b][1,8]naphthyridin-6(7H)-one. (7B)

### (5S,9R)-5-ethyl-9-fluoro-8,8-dimethyl-5-phenyl-5,8,9,10-tetrahydrobenzo[b][1,8]naphthyridin-6(7H)-one. (7C)

### (5S,9S)-5-ethyl-9-fluoro-8,8-dimethyl-5-phenyl-5,89,10-tetrahydrobenzo[b][1,8]naphthyridin-6(7H)-one. (7D)

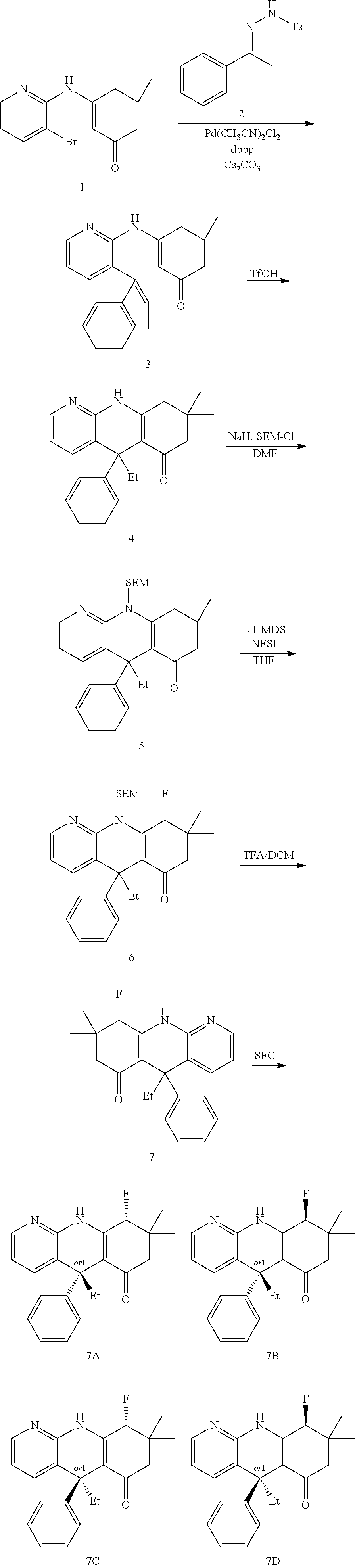

### Step 1: (F_)-5,5-dimethyl-3-((3-(1-phenylprop-1-en-1-yl)pyridin-2-yl)amino)cyclohex-2-en-1-one. (3)

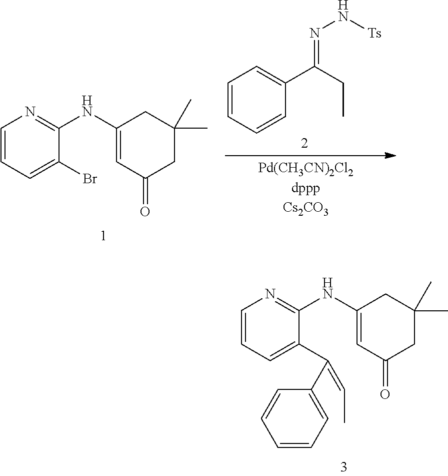

Under N₂, a mixture of compound 1 (2.0 g, 6.78 mmol), compound 2 (4.1 g, 13.56 mmol) Pd(CH₃CN)₂Cl₂ (176 mg, 0.68 mmol), dppp (280 mg, 0.68 mmol), Cs₂CO₃ (5.52 g, 16.95 mmol) in dioxane (50 mL) was stirred at 100° C. for 16 hours. TLC (EtOAc) showed starting material was consumed and a new spot observed. The reaction suspension was diluted with water (200) mL) and extracted with EtOAc (100 mL×2). The combined organic layer was washed with water (100 mL) and brine (100 mL), dried over Na₂SO₄, filtered. The filtrate was concentrated, and the residue was purified by silica gel (EtOAc) to afford compound 3 (1.2 g, 53.24% yield) as a yellow solid.

LCMS: (ES⁺) M/Z (M+H)⁺: 333.0.

### Step 2: 5-ethyl-8,8-dimethyl-5-phenyl-5,8,9,10-tetrahydrobenzo[b][1,8]naphthyridin-6(7H)-one. (4)

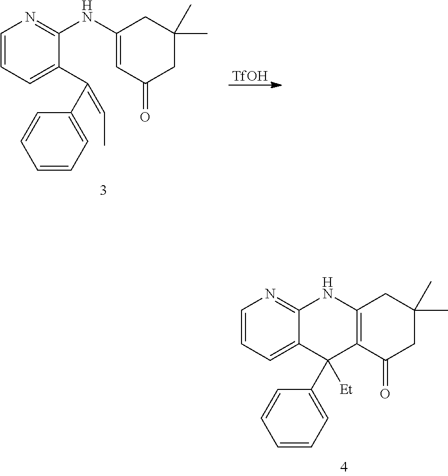

A solution of compound 3 (1.10 g, 3.31 mmol) in TfOH (10 mL) was stirred at 20° C. for 16 hours. TLC (Petroleum ether/EtOAc=1/1) showed starting material was consumed and a new spot was observed. The reaction solution was added into NaOH (4N, 100 mL) under ice cooling. The suspension was extracted with EtOAc (150 mL). The organic layer was washed with water (100 mL) and brine (100 mL), dried over Na₂SO₄, and filtered. The filtrate was concentrated, and the residue was purified by silica gel (Petroleum ether/EtOAc=1/1) to afford compound 4 (800 mg, 72.70% yield) as a white solid.

¹H NMR: (400 MHz, methanol-d4) δ=7.92 (dd, J=1.6, 4.8 Hz, 1H), 7.42-7.32 (m, 2H), 7.19 (t, J=7.6 Hz, 2H), 7.09-7.00 (m, 2H), 6.77 (dd, J=4.8, 7.6 Hz, 1H), 3.06-2.91 (m, 1H), 2.60-2.50 (m, 2H), 2.16-2.00 (m, 3H), 1.10 (d, J=11.6 Hz, 6H), 0.76 (t, J=7.2 Hz, 3H).

### Step 3: 5-ethyl-8,8-dimethyl-5-phenyl-5,8,9,10-tetrahydrobenzo[b][1,8]naphthyridin-6(7H)-one. (5)

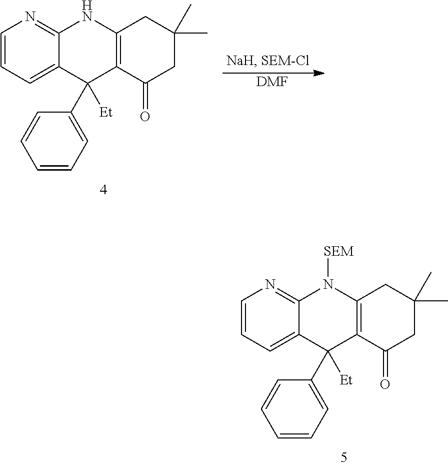

Under ice cooling, to a solution of compound 4 (940 mg, 2.83 mmol) in DMF (15.0 mL) was added NaH (2260 mg, 5.66 mmol, 60% purity) at 5° C. The reaction suspension was stirred at 5° C. for 1 hour. Then SEM-Cl (944 mg, 5.66 mmol, 1.0 mL) was added at 5° C. The red reaction suspension was stirred at 5° C. for 4 hours. TLC (Petroleum ether/EtOAc=1/1) showed starting material was consumed and a new spot was observed. The reaction was quenched with water (150 mL) and extracted with EtOAc (150 mL). The organic layer was washed with water (100 mL) and brine (100 mL), dried over Na₂SO₄, filtered. The filtrate was concentrated, and the residue was purified by silica gel (Petroleum ether/EtOAc=9/1) to afford compound 5 (1.0 g, 76.4% yield) as a yellow oil.

¹H NMR: (400 MHz, chloroform-d) δ=8.11-8.00 (m, 1H), 7.41 (d, J=7.6 Hz, 2H), 7.28-7.21 (m, 2H), 7.15-7.07 (m, 1H), 7.07-6.98 (m, 1H), 6.83-6.73 (m, 1H), 5.96-5.44 (m, 2H), 3.76-3.63 (m, 2H), 3.16-3.01 (m, 1H), 2.95-2.77 (m, 2H), 2.21-2.08 (m, 2H), 2.02-1.89 (m, 1H), 1.32-1.26 (m, 2H), 1.14 (d, J=14.8 Hz, 6H), 0.82-0.69 (m, 3H), −0.03 (br d, J=2.4 Hz, 1H), 0.07-−0.03 (m, 9H).

### Step 4: 5-ethyl-9-fluoro-8,8-dimethyl-5-phenyl-10-((2-(trimethylsilyl)ethoxy)methyl)-5,8,9,10-tetrahydrobenzo[b][1,8]naphthyridin-6(7H)-one. (6)

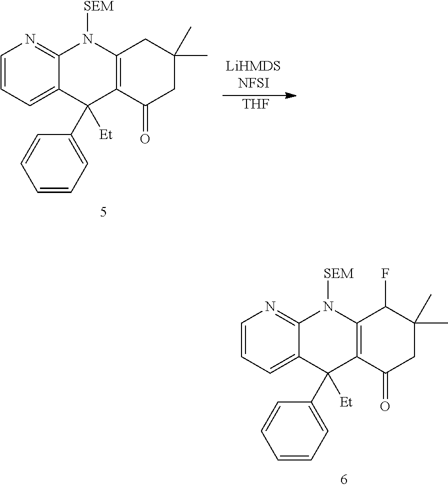 [SMILES: CCC1(c2ccccc2)C2=C(CC(C)(C)CC2=O)N(COCC[Si](C)(C)C)c2ncccc21 ; CCC1(c2ccccc2)C2=C(C(F)C(C)(C)CC2=O)N(COCC[Si](C)(C)C)c2ncccc21 ; "THF"]

Under N₂ and EtOH-dry ice cooling, to a solution of compound 5 (900 mg, 1.95 mmol) in THF (20 mL) was added LiHMDS (1 M in THF, 2.5 mL) at −65° C. The reaction solution was stirred at −65° C. for 1 hour. Then another solution of NFSI (615 mg, 1.95 mmol) in THF (5 mL) was added at −65° C. The reaction solution was stirred at −65° C.-20° C. for 16 hours. TLC (PE/EtOAc=7/1) showed new spots were observed and starting material remained. The reaction solution was quenched with water (100 mL) and extracted with EtOAc (50 mL×3). The combined organic layer was washed with water (50 mL) and brine (50 mL), dried over Na₂SO₄, filtered. The filtrate was concentrated, and the residue was purified by silica gel (Petroleum ether/EtOAc=9/1) to afford compound 6 (500 mg, 53.33% yield) as a yellow oil.

LCMS: (ES⁺) M/Z (M+H)⁺: 481.1.

### Step 5: (5R,9R)-5-ethyl-9-fluoro-8,8-dimethyl-5-phenyl-5,8,9,10-tetrahydrobenzo[b][1,8]naphthyridin-6(7H)-one. (7A)

### (5R,9S)-5-ethyl-9-fluoro-8,8-dimethyl-5-phenyl-5,8,9,10-tetrahydrobenzo[b][1,8]naphthyridin-6(7H)-one. (7B)

### (5S,9R)-5-ethyl-9-fluoro-8,8-dimethyl-5-phenyl-5,8,9,10-tetrahydrobenzo[b][1,8]naphthyridin-6(7H)-one. (7C)

### (5S,9S)-5-ethyl-9-fluoro-8,8-dimethyl-5-phenyl-5,8,9,10-tetrahydrobenzo[b][1,8]naphthyridin-6(7H)-one. (7D)

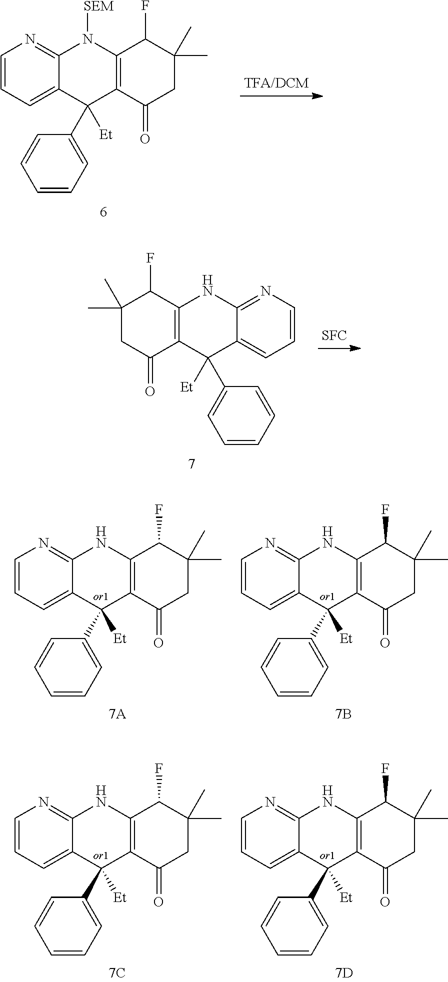

A solution of compound 6 (500 mg, 1.04 mmol) in DCM (4.0 mL) and TFA (2.0 mL) was stirred at 20° C. for 2 hours. LCMS showed desired m/z observed. The reaction solution was concentrated, and the residue was purified by prep-HPLC (Column: Xtimate C18 150×25 mm×5 μm; Condition: water (10 mM NH₄HCO₃)-ACN) to afford compound 7 (200 mg, 54.9% yield) as a yellow solid.

LCMS: tR=2.997 min., (ES⁺) M/Z (M+H)⁺: 351.0.

¹H NMR (400 MHz, chloroform-d) δ=8.00 (d, J=4.4 Hz, 1H), 7.49-7.34 (m, 3H), 7.26-7.22 (m, 2H), 7.15-7.08 (m, 1H), 7.07-7.00 (m, 1H), 6.84-6.75 (m, 1H), 5.33-5.14 (m, 1H), 3.16-2.92 (m, 1H), 2.34-2.12 (m, 2H), 2.06-1.88 (m, 1H), 1.22-1.08 (m, 6H), 0.86-0.73 (m, 3H). The racemic material was separated by SFC (Column: DAICEL CHIRALCEL OD (250 mm×30 mm, 10 μm); Condition: 0.1% NH₃H₂O EtOH, 30%, Flow rate: 70 mL/min) to afford:

(5R,9R)-5-ethyl-9-fluoro-8,8-dimethyl-5-phenyl-5,8,9,10-tetrahydrobenzo[b][1,8]naphthyridin-6(7H)-one. (7A) (25.0 mg, 0.07 mmol, 12.50% yield).

LCMS: tR=3.016 min., (ES⁺) M/Z (M+H)⁺: 351.1.

HPLC: (purity: 97.02%).

¹H NMR: (400 MHz, methanol-d4) δ=8.01 (dd, J=1.6, 4.8 Hz, 1H), 7.41 (d, J=7.2 Hz, 2H), 7.26 (t, J=2H), 7.15-7.06 (m, 2H), 6.86 (dd, J=4.8, 7.6 Hz, H), 5.33-5.16 (m, 1H), 3.06-2.93 (m, 1H), 2.29-2.17 (m, 2H), 2.14-2.01 (m, 1H), 1.24-1.13 (m, 6H), 0.81 (t, J=7.2 Hz, 3H).

(5R,9S)-5-ethyl-9-fluoro-8,8-dimethyl-5-phenyl-5,8,9,10-tetrahydrobenzo[b][1,8]naphthyridin-6(7H)-one. (7B) (45.0 mg, 0.13 mmol, 22.50% yield).

LCMS: tR=3.310 min., (ES⁺) M/Z (M+H)⁺: 351.1.

HPLC: (purity: 95.34%).

¹H NMR: (400 MHz, methanol-d4) δ=7.98 (dd, J=1.6, 4.8 Hz, 1H), 7.37 (d, J=7.2 Hz, 2H), 7.22 (t, J=7.6 Hz, 2H), 7.12-7.04 (m, 2H), 6.84 (dd, J=4.8, 7.6 Hz, 1H), 5.28-5.11 (m, 1H), 3.04-2.92 (m, 1H), 2.31 (dd, J=4.0, 16.6 Hz, 1H), 2.13-2.03 (m, 2H), 1.16 (d, J=2.8 Hz, 6H), 0.81 (t, J=7.2 Hz, 3H).

(5S,9R)-5-ethyl-9-fluoro-8,8-dimethyl-5-phenyl-5,8,9,10-tetrahydrobenzo[b][1,8]naphthyridin-6(7H)-one. (7C) (46.0 mg, 0.13 mmol, 23.00% yield).

LCMS: tR=3.890 min., (ES⁺) M/Z (M+H)⁺: 351.0.

HPLC: (purity: 96.04%).

¹H NMR: (400 MHz, methanol-d4) δ=8.01 (dd, J=1.6, 4.8 Hz, 1H), 7.41 (d, J=7.2 Hz, 2H), 7.26 (t, J=7.6 Hz, 2H), 7.15-7.06 (m, 2H), 6.86 (dd, J=4.8, 7.6 Hz, 1H), 5.33-5.16 (m, 1H), 3.06-2.93 (m, 1H), 2.29-2.17 (m, 2H), 2.14-2.01 (m, 1H), 1.24-1.13 (m, 6H), 0.81 (t, J=7.2 Hz, 3H).

(5S,9S)-5-ethyl-9-fluoro-8,8-dimethyl-5-phenyl-5,8,9,10-tetrahydrobenzo[b][1,8]naphthyridin-6(7H)-one. (7D) (50.0 mg, 0.14 mmol, 25.0% yield) as a yellow solid.

LCMS: tR=4.704 min., (ES⁺) M/Z (M+H)⁺: 351.0.

HPLC: (purity: 96.50%).

¹H NMR: (400 MHz, methanol-d4) δ=7.98 (dd, J=1.6, 4.8 Hz, 1H), 7.37 (d, J=7.2 Hz, 2H), 7.22 (t, J=7.6 Hz, 2H), 7.12-7.04 (m, 2H), 6.84 (dd, J=4.8, 7.6 Hz, 1H), 5.28-5.11 (m, 1H), 3.04-2.92 (m, 1H), 2.31 (dd, J=4.0, 16.6 Hz, 1H), 2.13-2.03 (m, 2H), 1.16 (d, J=2.8 Hz, 6H), 0.81 (t, J=7.2 Hz, 3H).

### Example 2.3. (R)-5-ethyl-3-fluoro-8,8-dimethyl-5-phenyl-5,8,9,10-tetrahydrobenzo[b][1,8]naphthyridin-6(7H)-one. (5A)

### (S)-5-ethyl-3-fluoro-8,8-dimethyl-5-phenyl-5,8,9,10-tetrahydrobenzo[b][1,8]naphthyridin-6(7H)-one. (5B)

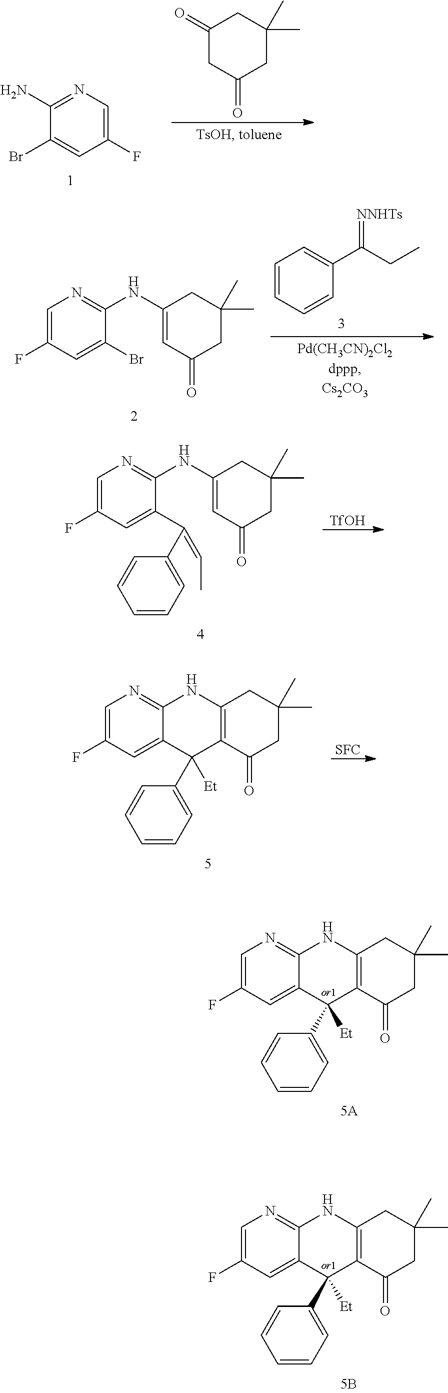

### Step 1: 3-((3-bromo-5-fluoropyridin-2-yl)amino)-5,5-dimethylcyclohex-2-en-1-one. (2)

To a 100 mL bottle with Dean-Stark was added compound 1 (4.6 g, 24.08 mmol), compound 1A (6.60 g, 47.08 mmol), TsOH·H₂O (916 mg, 4.82 mmol) and toluene (30 mL). The reaction suspension was stirred at 120° C. for 16 hours. LCMS showed desired m/z observed. The reaction solution was concentrated, and the residue was purified by silica gel (PE/EtOAc=1/1) to afford compound 2 (6.0 g, 51.72% yield) as a red oil.

LCMS: (ES⁺) M/Z (M+H)⁺: 313.1.

¹H NMR: (400 MHz, DMSO-d6) δ=8.51 (s, 1H), 8.42 (d, J=2.8 Hz, 1H), 8.25 (dd, J=2.8, 8.0 Hz, 1H), 5.84 (s, 1H), 2.44 (s, 2H), 2.05 (s, 2H), 0.99 (s, 6H).

### Step 2: (E)-3-((5-fluoro-3-(1-phenylprop-1-en-1-yl)pyridin-2-yl)amino)-5,5-dimethylcyclohex-2-en-1-one. (4)

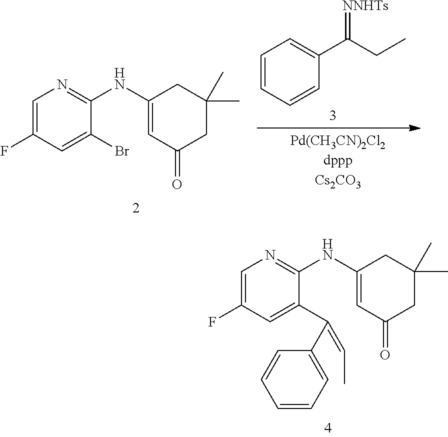

Under N₂, a mixture of compound 2 (1 g, 3.2 mmol), compound 3 (1.25 g, 4.15 mmol), Pd(CH3CN)₂Cl₂ (165 mg, 0.64 mmol), dppp (263 mg, 0.64 mmol), Cs₂CO₃ (2.1 g, 6.38 mmol) in dioxane (20 mL) was stirred at 90° C. for 16 hours. TLC (EtOAc) showed new spots observed. The reaction suspension was diluted with water (100 mL) and extracted with EtOAc (100 mL×2). The combined organic layer was washed with water (50 mL) and brine (50 mL), dried over Na₂SO₄, filtered. The filtrate was concentrated, and the residue was purified by silica gel (PE/EtOAc=1/1) to afford compound 4 (230 mg, 21% yield) as a red solid.

LCMS: (ES⁺) M/Z (M+H)⁺: 351.1.

### Step 3: (R)-5-ethyl-3-fluoro-8,8-dimethyl-5-phenyl-5,8,9,10-tetrahydrobenzo[b][1,8]naphthyridin-6(7H)-one. (5A)

### (S)-5-ethyl-3-fluoro-8,8-dimethyl-5-phenyl-5,8,9,10-tetrahydrobenzo[b][1,8]naphthyridin-6(7H)-one. (5B)

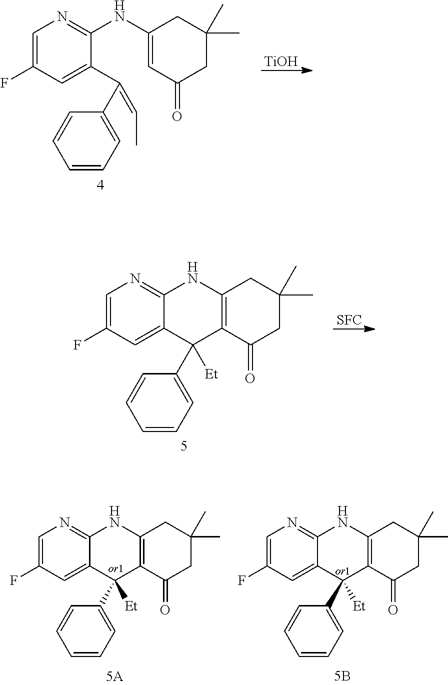

A solution of compound 4 (200 mg, 0.57 mmol) in TfOH (3 mL) was stirred at 90° C. for 1.5 hours. TLC (Petroleum ether/EtOAc=1/2) showed starting material was consumed and new spots observed. LCMS showed desired m/z observed. The reaction solution was added into NaOH (3N, 50 mL) dropwise. The suspension was extracted with EtOAc (50 mL×2). The combined organic layer was washed with water (100 mL) and brine (100 mL), dried over Na₂SO₄, filtered. The filtrate was concentrated, and the residue was purified by prep-HPLC (Column: Xtimate C18 150×25 mm×5 μm; Condition: water (10 mM NH₄HCO₃)-ACN) to afford 5 (50 mg, 0.14 mmol, 25% yield) as a yellow solid.

¹H NMR: (400 MHz, methanol-d4) δ=7.88 (d, J=2.8 Hz, 1H), 7.43-7.38 (m, 2H), 7.26 (t, J=7.6 Hz, 2H), 7.14-7.08 (m, 1H), 6.87 (dd, J=2.8, 9.2 Hz, 1H), 3.08-2.97 (m, 1H), 2.59 (s, 2H), 2.20-2.02 (m, 3H), 1.14 (d, J=10.8 Hz, 6H), 0.82 (t, J=7.2 Hz, 3H).

LCMS: (ES⁺) M/Z (M+H)⁺: 351.2.

HPLC: (purity: 100%).

The racemic material was separated by SFC (Column: DAICEL CHIRALPAK AS (250 mm×30 mm, 10 μm); Condition: 0.1% NH₃·H₂O EtOH, 30%, 80 mL/min) to afford:

(R)-5-ethyl-3-fluoro-8,8-dimethyl-5-phenyl-5,8,9,10-tetrahydrobenzo[b][1,8]naphthyridin-6(7H)-one. (5A) (19 mg, 0.05 mmol).

LCMS: tR=2.416 min., (ES⁺) M/Z (M+H)⁺=351.0.

HPLC: (purity: 99.90%).

¹H NMR: (400 MHz, methanol-d4) δ=7.84 (d, J=2.8 Hz, 1H), 7.37 (d, J=7.2 Hz, 2H), 7.22 (t, J=7.6 Hz, 2H), 7.10-7.03 (m, 1H), 6.83 (dd, J=2.8, 9.2 Hz, 1H), 3.05-2.92 (m, 1H), 2.54 (s, 2H), 2.16-2.00 (m, 3H), 1.10 (d, J=10.8 Hz, 6H), 0.78 (t, J=7.2 Hz, 3H).

(S)-5-ethyl-3-fluoro-8,8-dimethyl-5-phenyl-5,8,9,10-tetrahydrobenzo[b][1,8]naphthyridin-6(7H)-one. (5B) (18 mg, 0.05 mmol, 45% yield) both as white solid.

LCMS: tR=2.913 min., (ES⁺) M/Z (M+H)⁺=351.0.

HPLC: (purity: 97.17%).

¹H NMR: (400 MHz, methanol-d4) δ=7.84 (d, J=2.8 Hz, 1H), 7.37 (d, J=7.2 Hz, 2H), 7.22 (t, J=7.6 Hz, 2H), 7.10-7.03 (m, 1H), 6.83 (dd, J=2.8, 9.2 Hz, 1H), 3.05-2.92 (m, 1H), 2.54 (s, 2H), 2.16-2.00 (m, 3H), 1.10 (d, J=10.8 Hz, 6H), 0.78 (t, J=7.2 Hz, 3H).

### Example 2.4. (R)-5-ethyl-8,8-dimethyl-5-phenyl-3-(trifluoromethyl)-5,8,9,10-tetrahydrobenzo[b][1,8]naphthyridin-6(7H)-one. (5A)

### (S)-5-ethyl-8,8-dimethyl-5-phenyl-3-(trifluoromethyl)-5,8,9,10-tetrahydrobenzo[b][1,8]naphthyridin-6(7H)-one. (5B)

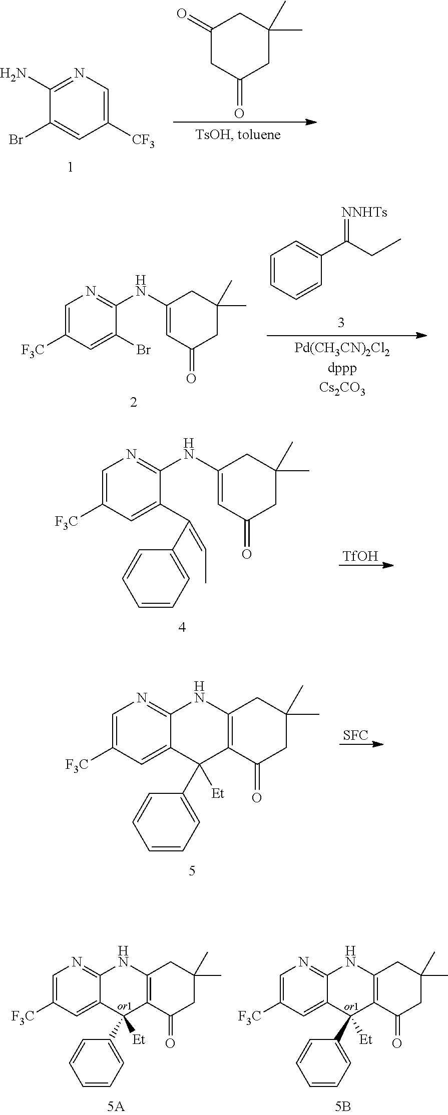

### Step 1: 3-((3-bromo-5-(trifluoromethyl)pyridin-2-yl)amino)-5,5-dimethylcyclohex-2-en-1-one. (2)

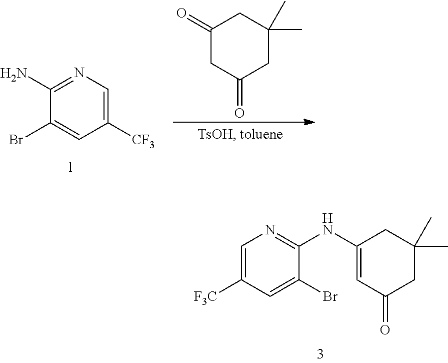

A suspension of compound 1 (1.20 g, 4.98 mmol), compound 1A (1.40 g, 9.96 mmol), TsOH (190 mg, 1.0 mmol) in toluene (30 mL) was stirred at 120° C. for 5 hours. TLC (PE/EtOAc=2/1 showed trace starting material remained and new spots observed. The reaction solution was diluted with EtOAc (100 mL). The solution was washed with saturated NaHCO₃ solution (100 mL), water (100 mL×2) and brine (100 mL), dried over Na₂SO₄, filtered. The filtrate was concentrated, and the residue was purified by silica gel (PE/EtOAc=4/1) to afford compound 2 (1.60 g, 88.5% yield) as a yellow solid.

LCMS: (ES⁺) M/Z (M+H)⁺: 365.1.

¹H NMR: (400 MHz, DMSO-d6) δ=8.70 (s, 1H), 8.55-8.46 (m, 2H), 6.56 (s, 1H), 2.58 (s, 2H), 2.12 (s, 2H), 1.02 (s, 6H).

### Step 2: (E)-5,5-dimethyl-3-((3-(1-phenylprop-1-en-1-yl)-5-(trifluoromethyl)pyridin-2-yl)amino)cyclohex-2-en-1-one. (4)

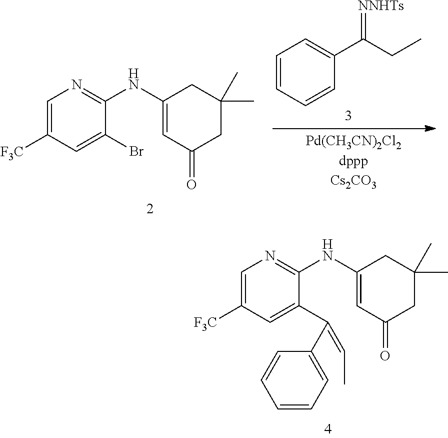

Under N₂, a mixture of compound 2 (800 mg, 2.20 mmol), compound 3 (700 mg, 2.31 mmol), Pd(CH₃CN)₂Cl₂ (114 mg, 0.44 mmol), dppp (182 mg, 0.44 mmol), Cs₂CO₃ (1.43 g, 4.40 mmol) in dioxane (10 mL) was stirred at 100° C. for 16 hours. LCMS showed desired m/z observed. The reaction suspension was concentrated to remove most solvent. The residue was diluted with water (100 mL) and extracted with EtOAc (70 mL×2). The combined organic layer was washed with water (100 mL) and brine (100 mL), dried over Na₂SO₄, filtered. The filtrate was concentrated, and the residue was purified by silica gel (PE/EtOAc=2/1) to afford compound 4 (350 mg, 39% yield) as a yellow oil.

LCMS: (ES⁺) M/Z (M+H)⁺: 401.3.

### Step 3: (R)-5-ethyl-8,8-dimethyl-5-phenyl-3-(trifluoromethyl)-5,8,9,10-tetrahydrobenzo[b][1,8]naphthyridin-6(7H)-one. (5A)

### (S)-5-ethyl-8,8-dimethyl-5-phenyl-3-(trifluoromethyl)-5,8,9,10-tetrahydrobenzo[b][1,8]naphthyridin-6(7H)-one. (5B)

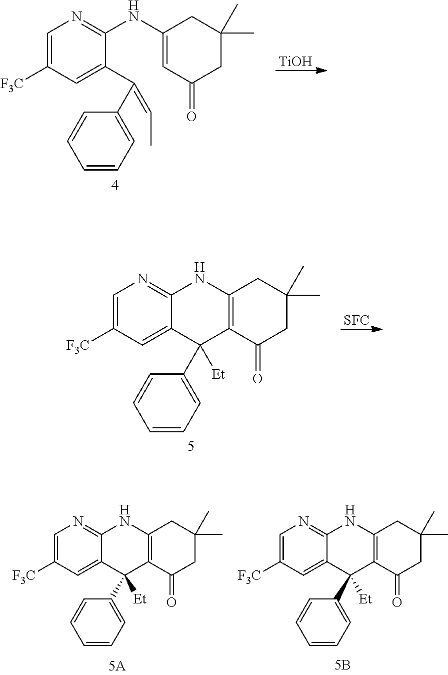

A solution of compound 4 (300 mg, 0.75 mmol) in TfOH (2.0 mL) was stirred at 90° C. for 1.5 h. LCMS showed desired m/z observed. TLC (EtOAc) showed starting material was consumed and new spots observed. The reaction solution was added into NaOH (4N, 50 mL). The suspension was extracted with EtOAc (100 mL×2). The combined organic layer was washed with water (100 mL) and brine (100 mL), dried over Na₂SO₄, filtered. The filtrate was concentrated. The residue was purified by prep-HPLC (Column: Xtimate C18 150×25 mm×5 μm; Condition: water (10 mM NH₄HCO₃)-ACN) to afford 5 (140 mg, 0.36 mmol) as a white solid.

LCMS: (ES+) M/Z (M⁺H)⁺: 401.1.

HPLC: (purity: 100%).

¹H NMR: (500 MHz, methanol-d4) δ=8.27 (s, 1H), 7.42 (d, J=8.0 Hz, 2H), 7.29 (t, J=7.5 Hz, 2H), 7.23 (s, 1H), 7.17-7.10 (m, 1H), 3.09-2.96 (m, 1H), 2.62 (d, J=2.5 Hz, 2H), 2.15 (q, J=16.0 Hz, 2H), 2.09-2.01 (m, 1H), 1.15 (d, J=13.0 Hz, 6H), 0.83 (t, J=7.0 Hz, 3H). The racemic 5 was separated by SFC (Column: DAICEL CHIRALCEL OD-H (250 mm×30 mm, 5 μm); Condition: 0.1% NH₃·H₂O EtOH, 25%, 60 mL/min) to afford:

(R)-5-ethyl-8,8-dimethyl-5-phenyl-3-(trifluoromethyl)-5,8,9,10-tetrahydrobenzo[b][1,8]naphthyridin-6(7H)-one. (5A) (60 mg, 0.05 mmol).

LCMS: tR=2.419 min., (ES⁺) M/Z (M+H)⁺: 401.0.

HPLC: (purity: 100%).

¹H NMR (400 MHz, methanol-d4) δ=8.25 (s, 1H), 7.39 (d, J=7.2 Hz, 2H), 7.26 (t, J=7.6 Hz, 2H), 7.20 (s, 1H), 7.13-7.06 (m, 1H), 3.06-2.95 (m, 1H), 2.59 (s, 2H), 2.18-2.07 (m, 2H), 2.07-1.98 (m, 1H), 1.12 (d, J=10.0 Hz, 6H), 0.80 (t, J=7.2 Hz, 3H).

(S)-5-ethyl-8,8-dimethyl-5-phenyl-3-(trifluoromethyl)-5,8,9,10-tetrahydrobenzo[b][1,8]naphthyridin-6(7H)-one. (5B) (60 mg, 0.05 mmol, 45% yield) both as white solid.

LCMS: tR=3.444 min., (ES⁺) M/Z (M+H)⁺=401.0.

HPLC: (purity: 100%).

¹H NMR: (400 MHz, methanol-d4) δ=8.25 (s, 1H), 7.39 (d, J=7.2 Hz, 2H), 7.26 (t, J=7.6 Hz, 2H), 7.20 (s, 1H), 7.13-7.06 (m, 1H), 3.06-2.95 (m, 1H), 2.59 (s, 2H), 2.18-2.07 (m, 2H), 2.07-1.98 (m, 1H), 1.12 (d, J=10.0 Hz, 6H), 0.80 (t, J=7.2 Hz, 3H).

### Example 2.5. (R)-5-ethyl-5-(3-hydroxyphenyl)-8,8-dimethyl-5,8,9,10-tetrahydrobenzo[b][1,8]naphthyridin-6(7H)-one. (5A)

### (S)-5-ethyl-5-(3-hydroxyphenyl)-8,8-dimethyl-5,8,9,10-tetrahydrobenzo[b][1,8]naphthyridin-6(7H)-one. (5B)

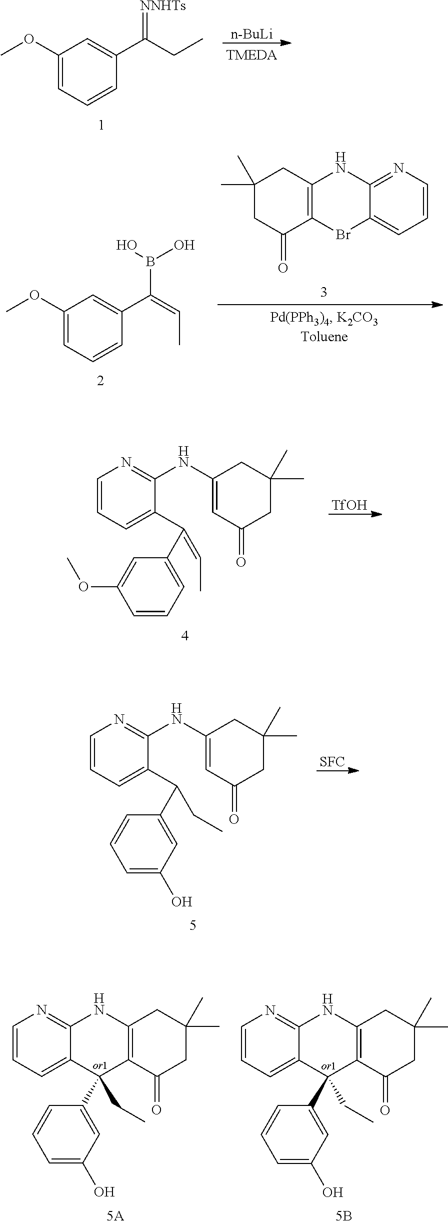

### Step 1: (Z)-(1-(3-methoxyphenyl)prop-1-en-1-yl)boronic acid. (2)

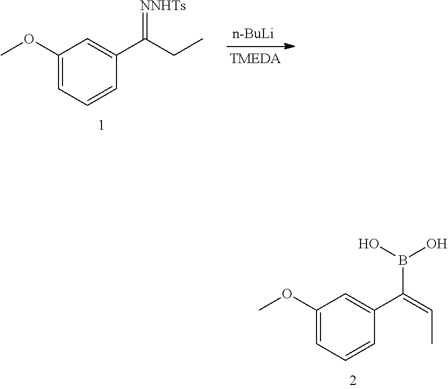

To a solution of compound 1 (1.00 g, 3.01 mmol) and compound TMEDA (1.08, 9.26 mmol) in THF (150.0) mL) was added n-BuLi (4.6 mL, 2.5 M) at −70° C. The mixture was stirred at −70° C. for 2 hours and warmed to 20° C. The resulting mixture was stirred at 20° C. for another 1 hour. Triisopropyl borate (2.82 g, 15.02 mmol) was added to the above solution at −70° C. The resulting mixture was stirred at 20° C. for 14 hours under N₂ atmosphere. TLC (PE:EtOAc=1:1) showed compound 1 was consumed completely. The mixture was concentrated adjusted to pH=4 with 8 M HCl. The organic layer was washed with 4M NaOH and the water layer was adjusted to pH=4 with 8 M HCl. The water layer was extracted with EtOAc (150.00 mL), washed with brine (150.00 mL). The organic layer was dried over Na₂SO₄, filtered and the filtrate was concentrated to get compound 2 (500 mg, crude) as a yellow oil. The oil was used to next step directly.

### Step 2: (E)-3-((3-(1-(3-methoxyphenyl)prop-1-en-1-yl)pyridin-2-yl)amino)-5,5-dimethylcyclohex-2-en-1-one. (3)

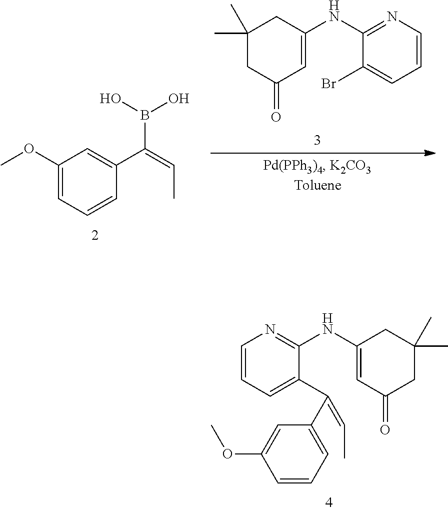

A mixture of compound 2 (500.00 mg, crude), compound 3 (767.00 mg, 2.60 mmol), K₂CO₃ (720 mg, 5.2 mmol) and Pd(PPh₃)₄ (3.00 g, 2.60 mmol) in toluene (5.00 mL) and water (5.00 mL) and EtOH (1.50 mL) was stirred at 90° C. under N₂ atmosphere for 14 hours. TLC (PE:EtOAc=1:1) showed the starting material remained and a main new spot was observed. The mixture was concentrated to get a residue, which was purified by pre-TLC (PE:EtOAc=1:1) to get compound 4 (300 mg, 31.83% yield) as a yellow solid.

LCMS: (ES⁺) M/Z (M+H)⁺: 349.0.

### Step 3: (R)-5-ethyl-5-(3-hydroxyphenyl)-8,8-dimethyl-5,8,9,10-tetrahydrobenzo[b][1,8]naphthyridin-6(7H)-one. (5A)

### (S)-5-ethyl-5-(3-hydroxyphenyl)-8,8-dimethyl-5,8,9,10-tetrahydrobenzo[b][1,8]naphthyridin-6(7H)-one. (5B)

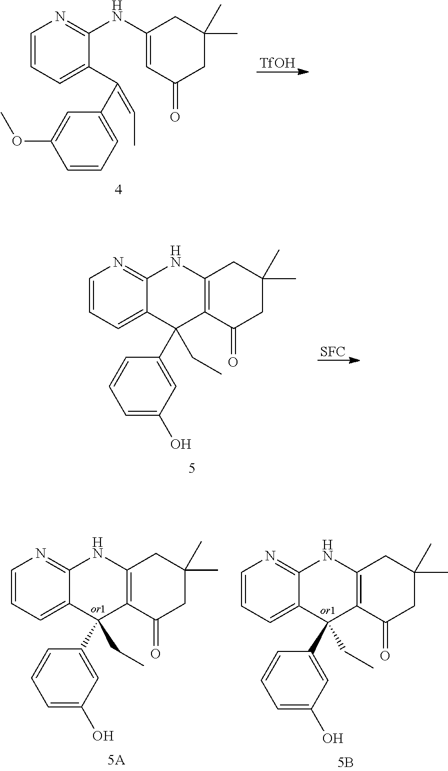

A solution of compound 4 (100.00 mg, 0.28 mmol) in TfOH (2.00 mL) was stirred at 90° C. for 3 hours under N₂. TLC (EtOAc:Petroleum ether=1:1) showed compound 4 was consumed completely and new spots were observed. The mixture was poured into saturated NaHCO₃ (2.00 mL) and extracted with EtOAc (2.00 mL). The organic layer was dried over Na₂SO₄, filtered and the filtrate was concentrated to get a residue, which was purified by flash column (Petroleum ether:EtOAc from 10/1 to 1/1) to get compound 5 (20.00 mg, 20.81% yield) as yellow solid.

LCMS: (ES⁺) M/Z (M+H)⁺: 348.9.

A racemic compound 5 (70.00 mg, 0.20 mmol) was separated by SFC (DAICEL Chiralpak AD-3 (250 mm×30 mm, 10 μm Mobile phase: 40% of ETOH (0.1% NH₃H₂O) in CO₂, Flow Rate (80 ml/min) to give:

(R)-5-ethyl-5-(3-hydroxyphenyl)-8,8-dimethyl-5,8,9,10-tetrahydrobenzo[b][1,8]naphthyridin-6(7H)-one. (5A) (25.00 mg, 35.71% yield) as a white solid.

LCMS: tR=1.423 min., (ES⁺) M/Z (M+H)⁺=349.0.

HPLC: (purity: 100%).

¹H NMR: (500 MHz, methanol-d4) δ=7.93-7.95 (m, 1H), 7.13-7.14 (m, 1H), 7.02-7.04 (m, 1H), 6.83-6.87 (m, 3H), 6.50-6.52 (m, 1H), 2.93-3.00 (m, 1H), 2.57 (s, 3H), 2.14-2.17 (m, 1H), 2.08-2.12 (m, 1H), 2.00-2.02 (m, 1H), 1.14 (s, 3H), 1.13 (s, 3H), 0.77 (t, J=7.5 Hz, 3H).

(S)-5-ethyl-5-(3-hydroxyphenyl)-8,8-dimethyl-5,8,9,10-tetrahydrobenzo[b][1,8]naphthyridin-6(7H)-one. (5B) (25.00 mg, 35.71% yield) as a white solid.

LCMS: tR=1.973 min., (ES⁺) M/Z (M+H)⁺=349.0.

HPLC: (purity: 100%).

¹H NMR: (500 MHz, methanol-d4) δ=7.83-7.84 (m, 1H), 7.03-7.04 (m, 1H), 6.92-6.94 (m, 1H), 6.70-6.77 (m, 3H), 6.41-6.42 (m, 1H), 2.82-2.89 (m, 1H), 2.46 (s, 3H), 2.04-2.07 (m, 1H), 1.98-2.01 (m, 1H), 1.89-1.90 (m, 1H), 1.04 (s, 3H), 1.02 (s, 3H), 0.66 (t, J=7.5 Hz, 3H).

### Example 2.6. (R)-5-ethyl-3-fluoro-5-(3-hydroxyphenyl)-8,8-dimethyl-5,8,9,10-tetrahydrobenzo[b][1,8]naphthyridin-6(7H)-one. (2A)

### (S)-5-ethyl-3-fluoro-5-(3-hydroxyphenyl)-8,8-dimethyl-5,8,9,10-tetrahydrobenzo[b][1,8]naphthyridin-6(7H)-one. (2B)

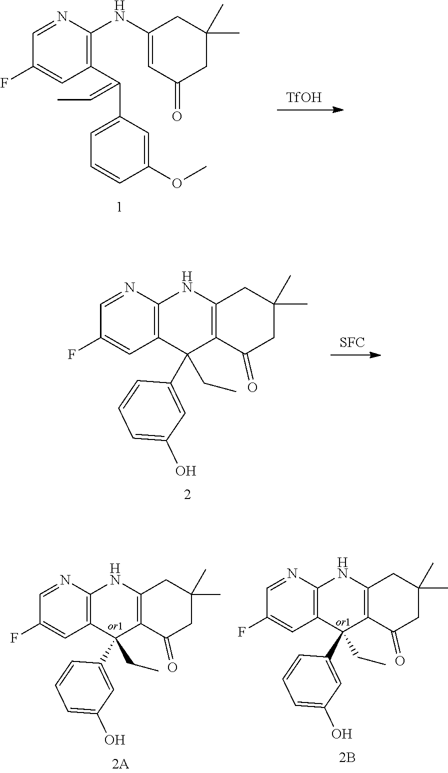

A mixture of compound 1 (500.00 mg, 1.31 mmol) in TfOH (20.40 g, 135.93 mmol, 12.00 mL) was stirred at 25° C. for 6 h. TLC (PE/EtOAc=1/1 254 nm) showed the reactant 1 was consumed and new spots were observed. The reaction mixture was concentrated, adjusted to pH=7 with NaOH (4 M) and extracted with EtOAc. The combined organic layer was dried over Na₂SO₄, filtered. The filtrate was concentrated, and the residue was purified by flash silica gel chromatography (PE/EtOAc=1/1) to 2 (186.00 mg, 38.75% yield) as a brown solid.

LCMS: (ES⁺) M/Z (M+H)⁺: 367.1.

¹H NMR: (500 MHz, methanol-d4) δ=7.86 (m, 1H), 7.09-7.03 (m, 1H), 6.93-6.82 (m, 3H), 6.54 (m, 1H), 3.01-2.94 (m, 1H), 2.56 (s, 2H), 2.01 (s, 3H), 1.13 (d, J=7.0 Hz, 6H), 0.78 (t, J=7.5 Hz, 3H).

The racemic 2 (186.00 mg) was separated by SFC (DAICEL CHIRALCEL OD-H (250 mm×30 mm, 5 μm); 0.1% NH₃H₂O ETOH; 30%; Flow Rate: 50 ml/min) to give:

(R)-5-ethyl-3-fluoro-5-(3-hydroxyphenyl)-8,8-dimethyl-5,8,9,10-tetrahydrobenzo[b][1,8]naphthyridin-6(7H)-one. (2A) (31.00 mg, 16.67% yield) as a yellow solid.

LCMS: tR=3.572 min., (ES⁺) M/Z (M+H)⁺: 367.1.

HPLC: (purity: 100%).

¹H NMR: (40 MHz, methanol-d4) δ=7.86 (d, J=2.8 Hz, 1H), 7.10-7.02 (m, 1H), 6.92-6.83 (m, 3H), 6.54 (m, 1H), 3.02-2.92 (m, 1H), 2.56 (s, 2H), 2.18-2.07 (m, 2H), 2.04-1.98 (m, 1H), 1.13 (d, J=5.2 Hz, 6H), 0.78 (t, J=7.2 Hz, 3H).

(S)-5-ethyl-3-fluoro-5-(3-hydroxyphenyl)-8,8-dimethyl-5,8,9,10-tetrahydrobenzo[b][1,8]naphthyridin-6(7H)-one. (2B) (38.00 mg, 20.43% yield) as a yellow solid.

LCMS: tR=4.510 min., (ES⁺) M/Z (M+H)⁺: 367.0.

HPLC: (purity: 100%).

¹H NMR: (500 MHz, methanol-d4) δ=7.86 (d, J=3.0 Hz, 1H), 7.06 (t, J=8.0 Hz, 1H), 6.91-6.83 (m, 3H), 6.54 (m, 1H), 3.02-2.92 (m, 1H), 2.56 (s, 2H), 2.17-2.08 (m, 2H), 2.04-1.96 (m, 1H), 1.13 (d, J=7.0 Hz, 6H), 0.78 (t, J=7.5 Hz, 3H).

### Example 2.7. (R)-5-ethyl-5-(3-isopropylphenyl)-8,8-dimethyl-5,8,9,10-tetrahydrobenzo[b][1,8]naphthyridin-6(7H)-one. (4A)

### (S)-5-ethyl-5-(3-isopropylphenyl)-8,8-dimethyl-5,8,9,10-tetrahydrobenzo[b][1,8]naphthyridin-6(7H)-one. (4B)

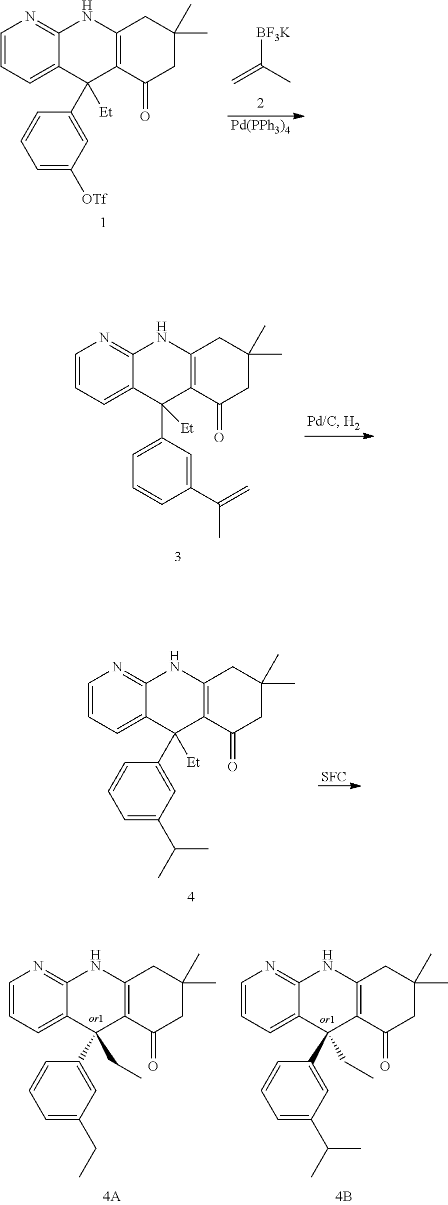

### Step 1: 5-ethyl-8,8-dimethyl-5-(3-(prop-1-en-2-yl)phenyl)-5,8,9,10-tetrahydrobenzo[b][1,8]naphthyridin-6(7H)-one. (3)

A mixture of compound 1 (100.00 mg, 0.21 mmol), compound 2 (47.00 mg, 0.32 mmol), K₂CO₃ (87 mg, 0.63 mmol) and Pd(PPh₃)₄ (24 mg, 0.021 mmol) in toluene (4.00 mL) was stirred at 90° C. under N₂ atmosphere for 16 hours. TLC (Petroleum ether:EtOAc=3:1) showed reactant 1 was consumed completely and a new spot was observed. The mixture was concentrated to get a residue, which was purified by pre-TLC (Petroleum ether:EtOAc=3/1) to get compound 3 (20 mg, 25.80% yield) as yellow oil. The oil was used to the next step directly.

### Step 2: (R)-5-ethyl-5-(3-isopropylphenyl)-8,8-dimethyl-5,8,9,10-tetrahydrobenzo[b][1,8]naphthyridin-6(7H)-one. (4A)

### (S)-5-ethyl-5-(3-isopropylphenyl)-8,8-dimethyl-5,8,9,10-tetrahydrobenzo[b][1,8]naphthyridin-6(7H)-one. (4B)

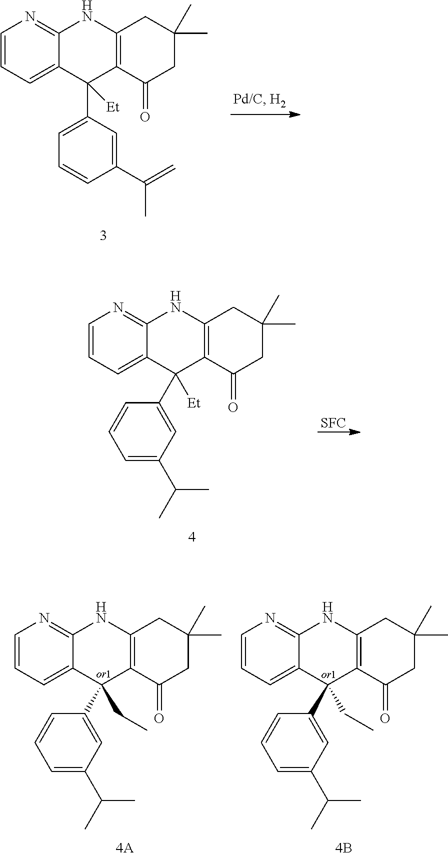

Pd/C (10 mg) was added to a solution of compound 2 (40.00 mg, 0.11 mmol) in MeOH (5.00 mL) under N₂ atmosphere. The mixture was stirred at H₂ 15 Psi for 16 hours. TLC (Petroleum ether:EtOAc=1:1) showed the starting material remained and new spots were observed. The mixture was concentrated to get a residue, which was purified by pre-TLC (Petroleum ether:EtOAc=1:1) to get a racemic compound 4 (30.00 mg).

The racemic compound was further purified by SFC (DAICEL CHIRALPAK OD (250 mm×30 mm, 10 μm), Mobile phase: A: CO₂, B: ETOH (0.1% NH₃H₂O), Flow Rate (60 ml/min) to give:

(R)-5-ethyl-5-(3-isopropylphenyl)-8,8-dimethyl-5,8,9,10-tetrahydrobenzo[b][1,8]naphthyridin-6(7H)-one. (4A) (8.00 mg, 26.67% yield) as a white solid.

LCMS: tR=1.019 min., (ES⁺) M/Z (M+H)⁺: 375.1.

HPLC: (purity: 99.29%).

¹H NMR: (400 MHz, methanol-d4) δ=7.93 (s, 1H), 7.20-7.22 (m, 2H), 7.14-7.15 (m, 1H), 7.07-7.10 (m, 1H), 6.94-6.95 (m, 1H), 6.80-6.81 (m, 1H), 2.99-3.00 (m, 1H), 2.80-2.81 (m, 1H), 2.57-2.63 (m, 2H), 2.15-2.19 (m, 1H), 2.03-2.06 (m, 2H), 1.12-1.19 (m, 12H), 0.76-0.80 (m, 3H).

(S)-5-ethyl-5-(3-isopropylphenyl)-8,8-dimethyl-5,8,9,10-tetrahydrobenzo[b][1,8]naphthyridin-6(7H)-one. (4B) (9.00 mg, 30.00% yield) as a white solid.

LCMS: tR=1.163 min., (ES⁺) M/Z (M+H)⁺: 375.1.

HPLC: (purity: 99.48%).

¹H NMR: (400 MHz, methanol-d4) δ=7.93-7.94 (m, 1H), 7.20-7.22 (m, 2H), 7.14-7.15 (m, 1H), 7.07-7.10 (m, 1H), 6.94-6.95 (m, 1H), 6.80-6.81 (m, 1H), 2.99-3.03 (m, 1H), 2.78-2.82 (m, 1H), 2.57-2.63 (m, 2H), 2.15-2.19 (m, 1H), 2.01-2.06 (m, 2H), 1.11-1.18 (m, 12H), 0.76-0.79 (m, 3H).

### Example 2.8. 5-(3-cyclobutylphenyl)-5-ethyl-8,8-dimethyl-5,8,9,10-tetrahydrobenzo[b][1,8]naphthyridin-6(7H)-one. (3)

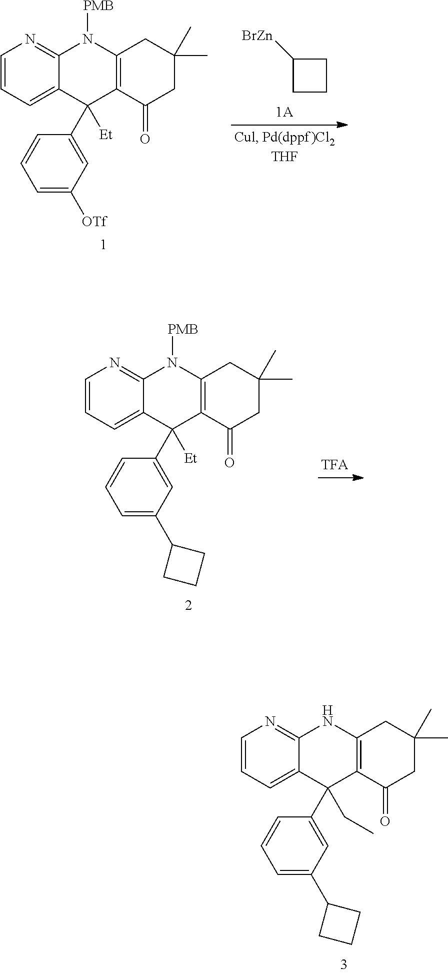

### Step 1: 5-(3-cyclobutylphenyl)-5-ethyl-10-(4-methoxy-benzyl)-8,8-dimethyl-5,8,9,10-tetrahydrobenzo[b][1,8]naphthyridin-6(7H)-one. (2)

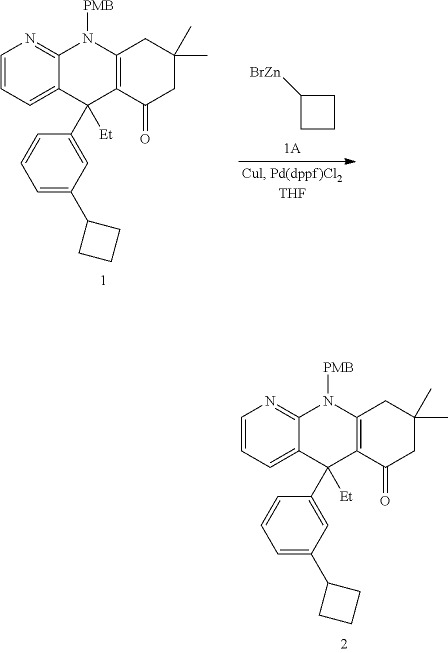

Under N₂, to a mixture of compound 1 (60 mg, 0.1 mmol), CuI (19 mg, 0.1 mmol), Pd(dppf)Cl₂ (73 mg, 0.1 mmol) in THF (3.0 mL) was added compound 1A (Sigma-Aldrich, 0.5 M in THF, 0.6 mL) at 20° C. The reaction suspension was stirred at 60° C. for 16 hours. TLC (PE/EtOAc=7/1) and LCMS showed starting material remained and new spots observed. The reaction suspension was concentrated, and the residue was purified by silica gel (Petroleum ether/EtOAc=9/1) to afford compound 2 (20 mg, 39.5% yield) as a colorless oil.

¹H NMR: (500 MHz, methanol-d4) δ=7.88 (dd, J=1.5, 4.5 Hz, 1H), 7.19-7.14 (m, 2H), 7.10-7.04 (m, 3H), 7.04-7.00 (m, 1H), 6.84 (d, J=7.5 Hz, 1H), 6.80 (d, J=8.5 Hz, 2H), 6.72 (dd, J=4.5, 7.5 Hz, 1H), 5.86-4.86 (m, 2H), 3.71-3.64 (m, 3H), 3.44-3.33 (m, 1H), 3.00-2.91 (m, 1H), 2.61 (q, J=16.8 Hz, 2H), 2.23-2.13 (m, 2H), 2.07-1.92 (m, 4H), 1.91-1.83 (m, 2H), 1.75-1.67 (m, 1H), 0.91-0.79 (m, 6H), 0.73-0.66 (m, 3H).

### Step 2: 5-(3-cyclobutylphenyl)-5-ethyl-8,8-dimethyl-5,8,9,10-tetrahydrobenzo[b][1,8]naphthyridin-6(7H)-one. (3)

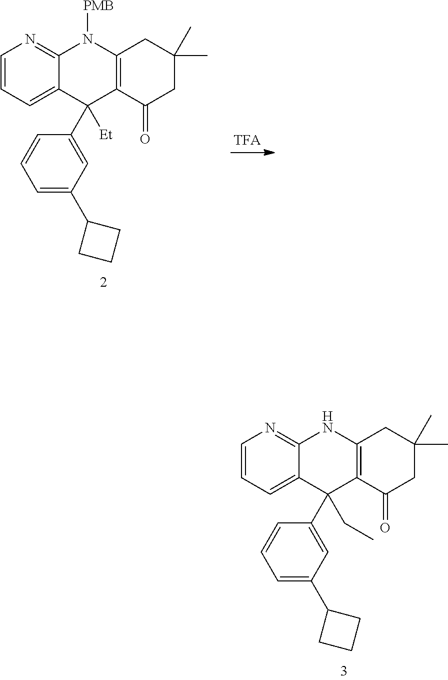

A solution of compound 2 (20 mg, 0.04 mmol) in TFA (3.0 mL) was stirred at 100° C. for 16 hours. LCMS showed desired m/z observed. The reaction solution was concentrated to dryness. The residue was purified by prep-HPLC (Column: YMC-Actus Triart C18 150×30 mm×5 μm; Condition: water (0.225% FA)-ACN) to afford 3 (4.0 mg, 26.22% yield) as a white solid.

LCMS: (ES⁺) M/Z (M+H)⁺: 387.0.

HPLC: (purity: 99.28%).

¹H NMR: (500 MHz, methanol-d4) δ=7.96 (dd, J=1.5, 4.5 Hz, 1H), 7.26-7.21 (m, 2H), 7.19-7.14 (m, 1H), 7.11 (dd, J=1.5, 7.5 Hz, 1H), 6.97 (d, J=7.5 Hz, 1H), 6.82 (dd, J=5.0, 7.5 Hz, 1H), 3.54-3.45 (m, 1H), 3.07-2.97 (m, 1H), 2.65-2.54 (m, 2H), 2.34-2.25 (m, 2H), 2.22-2.16 (m, 1H), 2.13-1.97 (m, 5H), 1.87-1.77 (m, 1H), 1.15 (d, J=9.0 Hz, 6H), 0.80 (t, J=7.5 Hz, 3H).

### Example 2.9. (R)-5-ethyl-8,8-dimethyl-5-(3-(trifluoromethyl)phenyl)-5,8,9,10-tetrahydrobenzo[b][1,8]naphthyridin-6(7H)-one. (6A)

### (S)-5-ethyl-8,8-dimethyl-5-(3-(trifluoromethyl)phenyl)-5,8,9,10-tetrahydrobenzo[b][1,8]naphthyridin-6(7H)-one. (6B)

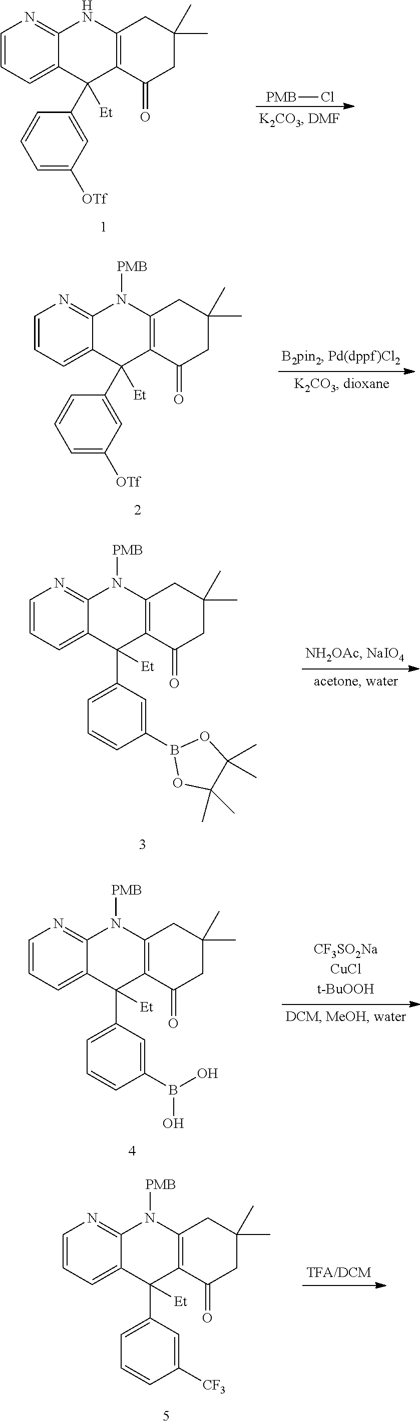

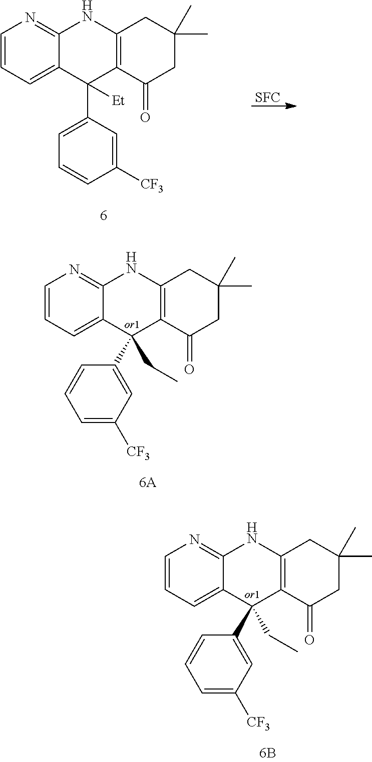 [SMILES: CCC1(c2cccc(C(F)(F)F)c2)C2=C(CC(C)(C)CC2=O)Nc2ncccc21 ; "SFC" ; CC[C@]1(c2cccc(C(F)(F)F)c2)C2=C(CC(C)(C)CC2=O)Nc2ncccc21 ; CC[C@@]1(c2cccc(C(F)(F)F)c2)C2=C(CC(C)(C)CC2=O)Nc2ncccc21]

### Step 1: 3-(5-ethyl-8,8-dimethyl-6-oxo-5,6,7,8,9,10-hexahydrobenzo[b][1,8]naphthyridin-5-yl)phenyl trifluoromethanesulfonate. (2)

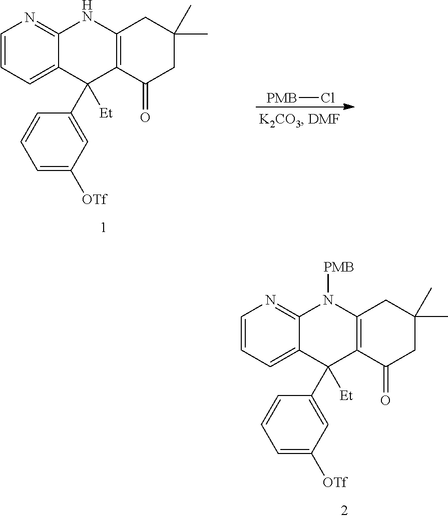

A solution of compound 1 (400 mg, 0.83 mmol) and PMB-Cl (130.37 mg, 0.83 mmol) in DMF (6.0 mL) was added K₂CO₃ (575 mg, 4.16 mmol) at 0° C. The resulting mixture was stirred at 100° C. for 1 hour. The mixture was poured into water (20 mL) and extracted with EtOAc (20 mL). The organic layer was washed with brine (20 mL), dried over Na₂SO₄, and filtered. The filtrate was concentrated, and the residue was purified by prep-TLC (petroleum ether/EtOAc=3/1) to get compound 2 (200 mg, 0.33 mmol, 40% yield) as a yellow oil.

### Step 2: 5-ethyl-10-(4-methoxybenzyl)-8,8-dimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one. (3)

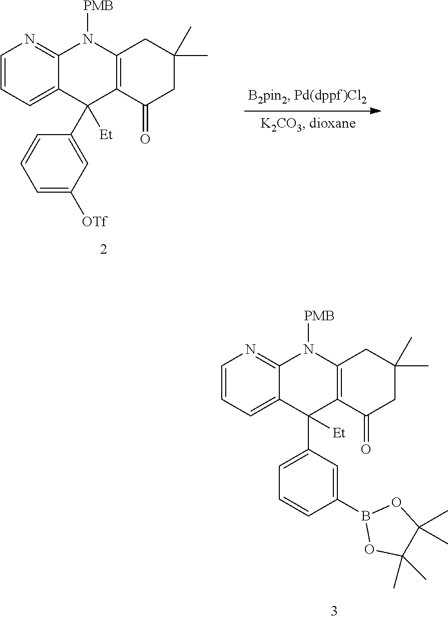

A mixture of compound 2 (200 mg, 0.33 mmol), B₂pin₂ (126 mg, 0.49 mmol), K₂CO₃ (138 mg, 0.99 mmol) and Pd(dppf)Cl₂ (24 mg, 0.03 mmol) in dioxane (10.0 mL) was stirred at 90° C. for 14 hours under N₂. TLC (petroleum ether/EtOAc=3/1) showed starting material was consumed completely and a main new spot was detected. The mixture was concentrated, and the residue was purified by silica gel (petroleum ether/EtOAc=10/1 to 3/1) to get compound 3 (100 mg, 0.17 mmol, 51% yield) as a yellow oil.

### Step 3: (3-(5-ethyl-10-(4-methoxybenzyl)-8,8-dimethyl-6-oxo-5,6,7,8,9,10-hexahydrobenzo[b][1,8]naphthyridin-5-yl)phenyl)boronic acid. (4)

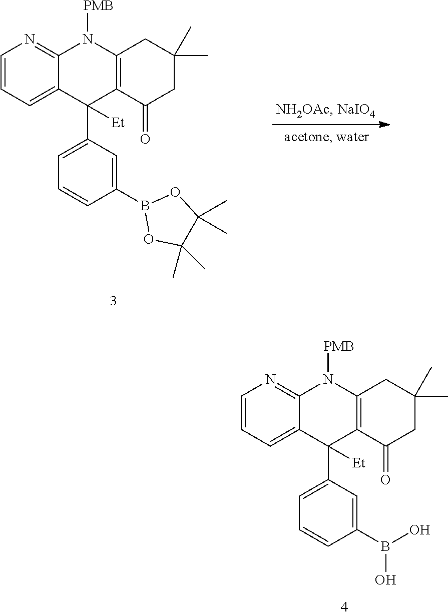

To a mixture of compound 3 (50 mg, 0.16 mmol) and NH₄OAc (39.97 mg, 0.52 mmol) in acetone (2.0 mL) and water 2.0 mL) was added NaIO₄ (55.5 mg, 0.26 mmol). The reaction was stirred at 35° C. for 6 hours. The acetone was removed, and the residue was dissolved in EtOAc (10 mL). The suspension was washed with 1N aq. HCl (5 mL). The organic layer was separated and concentrated in vacuo to get compound 4 (40 mg, 0.08 mmol, 93.2% yield) as a yellow solid.

### Step 4: 5-ethyl-10-(4-methoxybenzyl)-8,8-dimethyl-5-(3-(trifluoromethyl)phenyl)-5,8,9,10-tetrahydrobenzo[b][1,8]naphthyridin-6(7H)-one. (5)

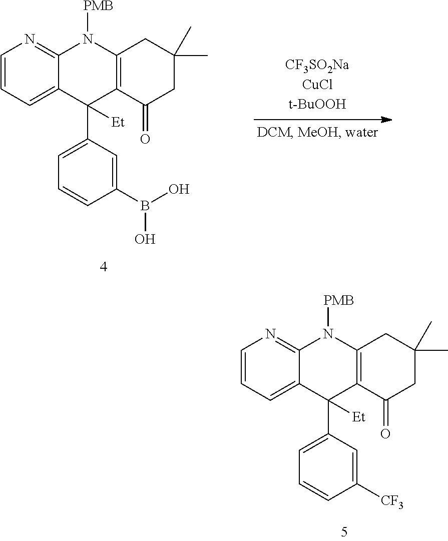

To a mixture of compound 4 (60 mg, 0.12 mmol), CF₃SO₂Na (56 mg, 0.36 mmol) and CuCl (11 mg, 0.12 mmol) in DCM (1 mL), MeOH (1 mL) and water (0.60 mL) was added t-BuOOH (54 mg, 0.60 mmol) over 10 mins. The reaction was stirred at 18° C. for 16 hours. TLC (Petroleum ether/EtOAc=3/1) showed reactant 1 was consumed completely and a new spot was detected. The mixture was concentrated, and the residue was purified by prep TLC (Petroleum ether/EtOAc=1/1) to get compound 5 (55 mg, 0.10 mmol, 87% yield) as a yellow oil.

### Step 5: (R)-5-ethyl-8,8-dimethyl-5-(3-(trifluoromethyl)phenyl)-5,8,9,10-tetrahydrobenzo[b][1,8]naphthyridin-6(7H)-one. (6A)

### (S)-5-ethyl-8,8-dimethyl-5-(3-(trifluoromethyl)phenyl)-5,8,9,10-tetrahydrobenzo[b][1,8]naphthyridin-6(7H)-one. (6B)

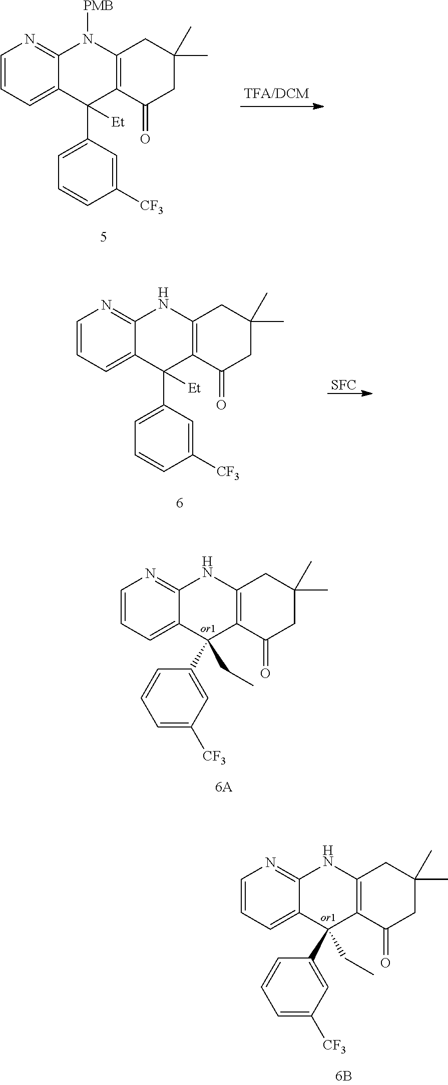

To a solution of compound 5 (55 mg, 0.10 mmol) in DCM (1.0 mL) was added TFA (1.0 mL). The mixture was stirred at 25° C. for 14 hours. The mixture was concentrated, and the residue was purified by prep TLC (petroleum ether/EtOAc=1/1) to get 6 (30 mg, 0.07 mmol, 70% yield) as a yellow solid.

The racemic 6 (30 mg, 0.07 mmol) was separated by SFC (DAICEL CHIRALPAK AD-H (250 mm×30 mm, 10 μm), Mobile phase: A: CO₂, B: EtOH (0.1% NH₃H₂O), Flow Rate (60 mL/min), Flow Rate (60 mL/min) to give:

(R)-5-ethyl-8,8-dimethyl-5-(3-(trifluoromethyl)phenyl)-5,8,9,10-tetrahydrobenzo[b][1,8]naphthyridin-6(7H)-one. (6A) (8.0 mg, 0.01 mmol, 26% yield, 98% purity) as a white solid.

LCMS: (ES⁺) M/Z (M+H)⁺=401.1.

HPLC: (purity: 97.97%).

¹H NMR: (500 MHz, methanol-d4) δ ppm: 7.88-7.93 (m, 1H), 7.53-7.61 (m, 2H), 7.25-7.36 (m, 2H), 6.92-6.99 (m, 1H), 6.71-6.78 (m, 1H), 2.85-2.95 (m, 1H), 2.04-2.10 (m, 1H), 1.89-1.99 (m, 1H), 1.04 (s, 3H), 1.0) (s, 3H), 0.69 (t, J=7.0 Hz, 3H).

(S)-5-ethyl-8,8-dimethyl-5-(3-(trifluoromethyl)phenyl)-5,8,9,10-tetrahydrobenzo[b][1,8]naphthyridin-6(7H)-one. (6B) (8.0 mg, 0.01 mmol, 26% yield, 98% purity) as a white solid.

LCMS: (ES⁺) M/Z (M+H)⁺=401.1.

HPLC: (purity: 100.0%).

¹H NMR: (500 MHz, methanol-d4) δ ppm: 7.88-7.93 (m, 1H), 7.53-7.61 (m, 2H), 7.25-7.36 (m, 2H), 6.92-6.99 (m, 1H), 6.71-6.78 (m, 1H), 2.85-2.95 (m, 1H), 2.04-2.10 (m, 1H), 1.89-1.99 (m, 1H), 1.04 (s, 3H), 1.00 (s, 3H), 0.69 (t, J=7.0 Hz, 3H).

### Example 2.10. (R)-5-ethyl-8,8-dimethyl-5-phenyl-5,8,9,10-tetrahydropyrido[2,3-b][1,6]naphthyridin-6(7H)-one. (6A)

### (S)-5-ethyl-8,8-dimethyl-5-phenyl-5,8,9,10-tetrahydropyrido[2,3-b][1,6]naphthyridin-6(7H)-one. (6B)

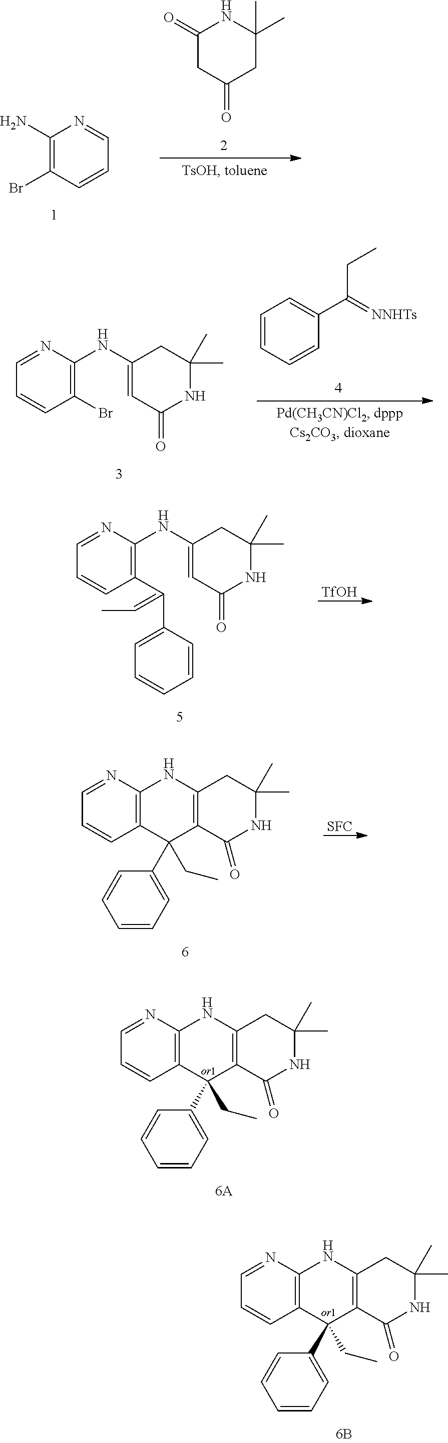

### Step 1: 4-((3-bromopyridin-2-yl)amino)-6,6-dimethyl-5,6-dihydropyridin-2(1H)-one. (2)

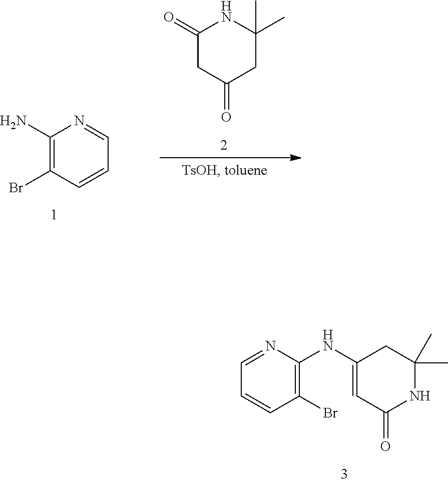

A mixture of compound 1 (4.5 g, 26.01 mmol), compound 2 (10.8 g, 76.47 mmol) and TsOH (544 mg, 2.86 mmol) in Toluene (150 mL) was stirred at 150° C. for 3 hours under N2 atmosphere. TLC (PE:EtOAc=1:1) showed the starting material remained and a new spot was detected. The reaction mixture was diluted with water (50 mL) extracted with EtOAc (50 mL×3). The combined organic layers were washed with brine (50 mL), dried over Na₂SO₄, filtered, and concentrated under reduced pressure to give residue. The residue was purified by flash silica gel chromatography (PE:EtOAc=1:1) to give compound 3 (1.25 g, 16.23% yield) as a yellow solid.

¹H NMR: (400 MHz, DMSO-d6) δ=8.33-8.23 (m, 1H), 7.99-8.01 (m J=1.2, 7.6 Hz, 1H), 7.85 (s, 1H), 6.96-6.77 (m, 2H), 6.20 (s, 1H), 2.52 (s, 2H), 1.17 (s, 6H).

### Step 2: (Z)-6,6-dimethyl-4-((3-(1-phenylprop-1-en-1-yl)pyridin-2-yl)amino)-5,6-dihydropyridin-2(1H)-one. (5)

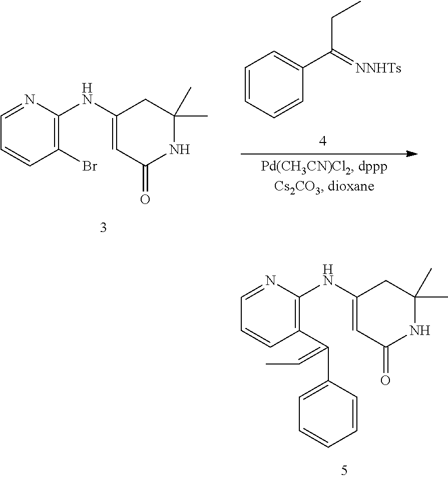

To a solution of compound 3 (600 mg, 2.03 mmol), compound 4 (1.23 g, 4.06 mmol) and Pd (CH₃CN)Cl₂ (53 mg, 0.2 mmol) in Dioxane (35 mL) was added dppp (84 mg, 0.2 mmol), Cs₂CO₃ (1.32 g, 4.06 mmol) stirred at 90° C. for 16 hours under N₂ atmosphere. TLC (EtOAc=100%) showed the starting material was remained and a new spot was detected. LCMS showed ˜58% of desired product MS was detected. The reaction mixture was diluted with water (50 mL) extracted with EtOAc (50 mL×3). The combined organic layers were washed with brine (50 mL), dried over Na₂SO₄, filtered, and concentrated under reduced pressure to give residue. The residue was purified by flash silica gel chromatography (EtOAc=100%) to give compound 5 (530 mg, 78.30% yield) as a yellow solid.

¹HNMR (400 MHz, MeOD) δ=8.35-8.33 (m, 1H), 7.73-7.65 (m, 1H), 7.57-7.53 (m, 1H), 7.52-7.46 (m, 1H), 7.29-7.07 (m, 7H), 6.48-6.43 (m, 1H), 6.27 (s, 1H), 2.21 (s, 2H), 1.69-1.74 (d, 3H), 1.22-1.09 (m, 7H).

### Step 3: (R)-5-ethyl-8,8-dimethyl-5-phenyl-5,8,9,10-tetrahydropyrido[2,3-b][1,6]naphthyridin-6(7H)-one. (6A)

### (S)-5-ethyl-8,8-dimethyl-5-phenyl-5,8,9,10-tetrahydropyrido[2,3-b][1,6]naphthyridin-6(7H)-one. (6B)

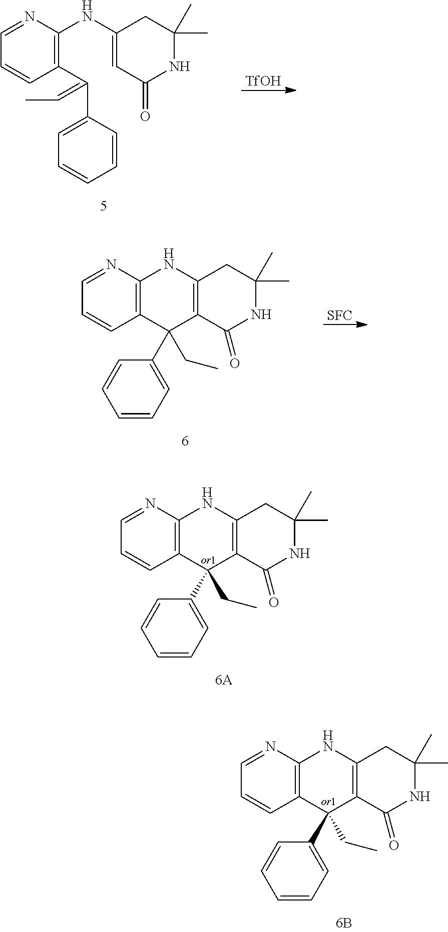

A mixture of compound 5 (100 mg, 0.30 mmol) in TfOH (1.7 g, 11.33 mmol, 1 mL) was stirred at 10-15° C. for 16 hours. TLC (EtOAc=100%) showed the starting material remained and new spots were detected. LCMS showed ˜26% of desired MS was detected. The residue was purified by flash silica gel chromatography (EtOAc=100%) to give compound 6 (70 mg, 70.00% yield) as a white solid.

¹H NMR: (400 MHz, MeOD) δ=7.90-7.89 (m, 1H), 7.40 (d, J=7.2 Hz, 2H), 7.22 (t, J=7.6 Hz, 2H), 7.10-7.02 (m, 2H), 6.77-6.74 (m, 1H), 3.09-2.97 (m, 1H), 2.70-2.52 (m, 2H), 2.12-2.00 (m, 1H), 1.33 (d, J=2.8 Hz, 6H), 0.82 (t, J=7.2 Hz, 3H). Compound 6 (65 mg, 0.20 mmol) was separated by SFC (DAICEL CHIRALCEL OD-H (250 mm×30 mm, 5 μm); 0.1% NH₃H₂O ETOH—CO₂; 40%, Flow Rate: 50 ml/min) to give:

(R)-5-ethyl-8,8-dimethyl-5-phenyl-5,8,9,10-tetrahydropyrido[2,3-b][1,6]naphthyridin-6(7H)-one. (6A) (22 mg, 33.85% yield).

LCMS: tR=3.779 min., (ES⁺) M/Z (M+H)⁺: 334.2.

HPLC: (purity: 100%).

¹H NMR: (400 MHz, MeOD) δ=7.91-7.89 (m, 1H), 7.45-7.34 (m, 2H), 7.28-7.18 (m, 2H), 7.12-7.01 (m, 2H), 6.77-6.74 (m, 1H), 3.08-2.99 (m, 1H), 2.71-2.51 (m, 2H), 2.13-1.99 (m, 1H), 1.33 (d, J=2.8 Hz, 6H), 0.82 (t, J=7.3 Hz, 3H).

(S)-5-ethyl-8,8-dimethyl-5-phenyl-5,8,9,10-tetrahydropyrido[2,3-b][1,6]naphthyridin-6(7H)-one. (6B).

(27 mg, 41.54% yield) both as white solid.

LCMS: tR=5.932 min., (ES⁺) M/Z (M+H)⁺: 334.1.

HPLC: (purity: 100%).

¹H NMR: (400 MHz, MeOD) δ=7.91-7.89 (m, 1H), 7.40 (d, J=7.2 Hz, 2H), 7.23 (t, J=7.6 Hz, 2H), 7.13-7.01 (m, 2H), 6.77-6.74 (m, 1H), 3.11-2.95 (m, 1H), 2.71-2.52 (m, 2H), 2.14-2.00 (m, 1H), 1.33 (d, J=2.8 Hz, 6H), 0.82 (t, J=7.2 Hz, 3H).

### Example 2.11. (R)-5-ethyl-3-fluoro-8,8-dimethyl-5-phenyl-5,8,9,10-tetrahydropyrido[2,3-b][1,6]naphthyridin-6(7H)-one. (6A)

### (S)-5-ethyl-3-fluoro-8,8-dimethyl-5-phenyl-5,8,9,10-tetrahydropyrido[2,3-b][1,6]naphthyridin-6(7H)-one. (6B)

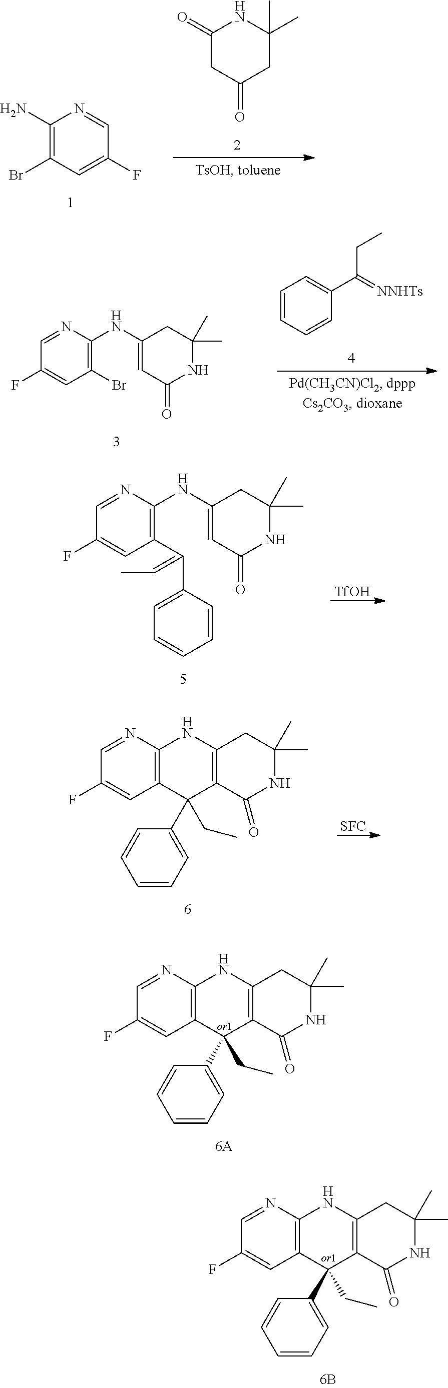

### Step 1: 4-((3-bromo-5-fluoropyridin-2-yl)amino)-6,6-dimethyl-5,6-dihydropyridin-2(1H)-one. (3)

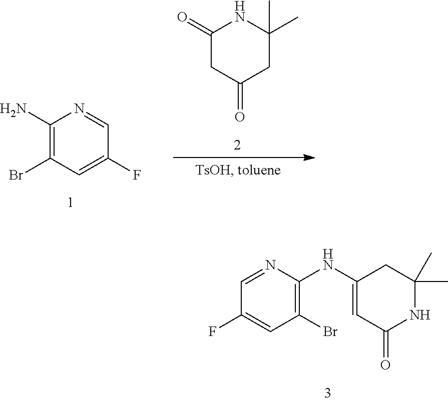

A solution of compound 1 (800.00 mg, 4.19 mmol), compound 2 (650.42 mg, 4.61 mmol) and TsOH (65.48 mg, 1.26 mmol) in Toluene (12.00 mL) was stirred at 140° C. for 5 hours. TLC (EtOAc 254 nm) showed starting material 1 remained and new spots observed. The reaction solution was concentrated, and the residue was purified by silica gel (EtOAc/MeOH=9/1) to afford compound 3 (1.30 g, 98.76% yield) as a yellow solid.

¹H NMR: (500 MHz, methanol-d4) δ=8.26 (d, J=2.5 Hz, 1H), 7.96 (m, 1H), 6.40 (s, 1H), 2.64 (s, 2H), 1.35 (s, 6H).

### Step 2: (Z)-4-((5-fluoro-3-(1-phenylprop-1-en-1-yl)pyridin-2-yl)amino)-6,6-dimethyl-5,6-dihydropyridin-2(1H)-one. (5)

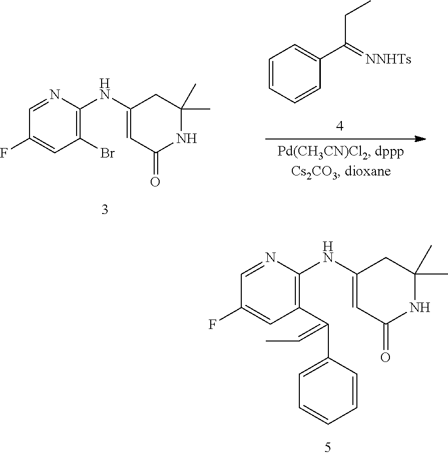

Compound 3 (300.00 mg, 954.96 μmol), compound 4 (404.28 mg, 1.34 mmol) and Cs₂CO₃ (622.29 mg, 1.91 mmol) were added into Dioxane (8.00 mL). The reaction suspension was bubbled with N₂ for 1 min, then Pd(CH₃CN)₂Cl₂ (24.77 mg, 95.50 μmol) and dppp (39.39 mg, 95.50 μmol) were added. The reaction mixture was stirred at 90° C. for 16 h under N₂ atmosphere. TLC (EtOAc/MeOH=19/1, 254 nm) showed the reactant 3 was not consumed completely and new spots were observed. The reaction mixture was concentrated and extracted with EtOAc. The combined organic layer was dried over Na₂SO₄, filtered. The filtrate was purified by flash silica gel chromatography (EtOAc/MeOH=19/1) to give compound 5 (183.00 mg, 54.53% yield) as a brown solid.

LCMS: (ES⁺) M/Z (M+H)⁺: 352.0.

### Step 3: (R)-5-ethyl-3-fluoro-8,8-dimethyl-5-phenyl-5,8,9,10-tetrahydropyrido[2,3-b][1,6]naphthyridin-6(7H)-one. (6A)

### (S)-5-ethyl-3-fluoro-8,8-dimethyl-5-phenyl-5,8,9,10-tetrahydropyrido[2,3-b][1,6]naphthyridin-6(7H)-one. (6B)

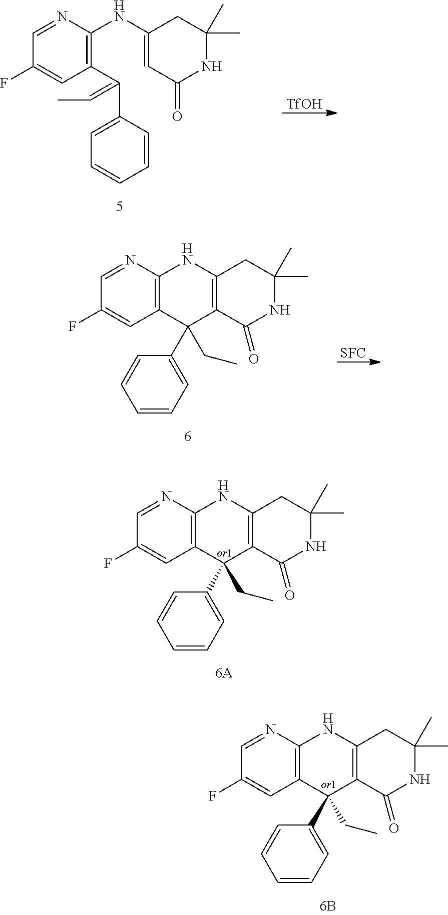

To a solution of compound 5 (183.00 mg, 520.74 μmol) in TfOH (5.00 mL) was stirred at 60° C. for 3 h. TLC (EtOAc/MeOH=19/1 254 nm) showed the reactant 1 was consumed and new spots were observed. The reaction mixture was concentrated, adjusted to pH=7 with NaOH (4 M) and extracted with EtOAc. The combined organic layer was dried over Na₂SO₄, filtered. The filtrate was concentrated. The residue was purified by flash silica gel chromatography (EtOAc/MeOH=19/1) and prep-HPLC (Column YMC-Actus Triart C18 100×30 mm×5 μm, Condition water (0.225% FA)-ACN) to give compound 6 (100.00 mg, 46.99% yield, 86% purity) as a white solid.

¹H NMR: (500 MHz, MeOD) δ=8.12 (s, 2H), 7.82 (d, J=3.0 Hz, 1H), 7.41 (d, J=7.5 Hz, 2H), 7.26 (t, J=8.0 Hz, 2H), 7.09 (t, J=7.5 Hz, 1H), 6.85 (m, 1H), 3.09-2.99 (m, 1H), 2.66-2.62 (m, 1H), 2.58-2.52 (m, 1H), 2.11-2.05 (m, 1H), 1.33 (d, J=3.0 Hz, 6H), 0.84 (t, J=7.5 Hz, 3H).

LCMS: (ES⁺) M/Z (M+H)⁺: 352.0.

Racemic 6 (100.00 mg, 284.56 μmol) was separated by SFC (DAICEL CHIRALCEL OD (250 mm×30 mm, 10 μm); 0.1% NH₃H₂O ETOH: 45%; Flow Rate: 60 ml/min) to give:

(R)-5-ethyl-3-fluoro-8,8-dimethyl-5-phenyl-5,8,9,10-tetrahydropyrido[2,3-b][1,6]naphthyridin-6(7H)-one. (6A) (6.00 mg, 6.00% yield) as a white solid.

LCMS: tR=1.071 min., (ES⁺) M/Z (M+H)⁺: 352.0.

HPLC: (purity: 100%).

¹H NMR: (500 MHz, methanol-d4) δ=7.82 (d, J=2.5 Hz, 1H), 7.41 (m, 2H), 7.28-7.23 (m, 2H), 7.12-7.06 (m, 1H), 6.85 (m, 1H), 3.08-2.99 (m, 1H), 2.67-2.53 (m, 2H), 2.14-2.03 (m, 1H), 1.33 (d, J=3.0 Hz, 6H), 0.84 (t, J=7.5 Hz, 3H).

(S)-5-ethyl-3-fluoro-8,8-dimethyl-5-phenyl-5,8,9,10-tetrahydropyrido[2,3-b][1,6]naphthyridin-6(7H)-one. (6B) (8.00 mg, 7.96% yield) as a white solid.

LCMS: tR=1.787 min., (ES⁺) M/Z (M+H)⁺: 352.0.

HPLC: (purity: 99.51%).

¹H NMR: (500 MHz, methanol-d4) δ=7.82 (d, J=2.5 Hz, 1H), 7.41 (m, 2H), 7.28-7.23 (m, 2H), 7.12-7.07 (m, 1H), 6.85 (m, 11H), 3.08-2.98 (m, 1H), 2.67-2.53 (m, 2H), 2.14-2.01 (m, 1H), 1.33 (d, J=3.0 Hz, 6H), 0.84 (t, J=7.5 Hz, 3H).

### Example 2.12. (R)-5-ethyl-8,8-dimethyl-5-(3-(pyridin-4-yl)phenyl)-5,8,9,10-tetrahydrobenzo[b][1,8]naphthyridin-6(7H)-one. (2A)

### (S)-5-ethyl-8,8-dimethyl-5-(3-(pyridin-4-yl)phenyl)-5,8,9,10-tetrahydrobenzo[b][1,8]naphthyridin-6(7H)-one. (2B)

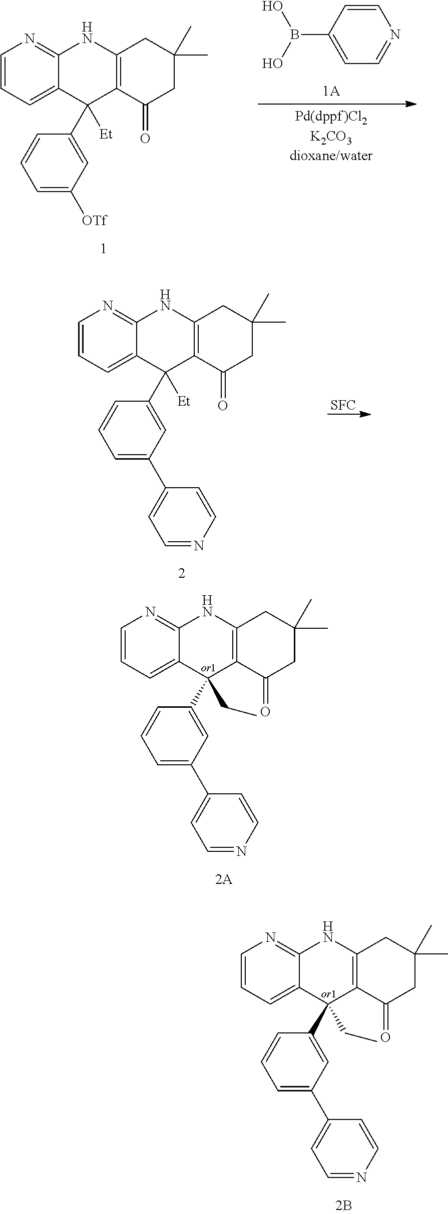 [SMILES: CCC1(c2cccc(OS(=O)(=O)C(F)(F)F)c2)C2=C(CC(C)(C)CC2=O)Nc2ncccc21 ; OB(O)c1ccncc1 ; CCC1(c2cccc(-c3ccncc3)c2)C2=C(CC(C)(C)CC2=O)Nc2ncccc21 ; "SFC" ; CC[C@]1(c2cccc(-c3ccncc3)c2)C2=C(CC(C)(C)CC2=O)Nc2ncccc21 ; CC[C@@]1(c2cccc(-c3ccncc3)c2)C2=C(CC(C)(C)CC2=O)Nc2ncccc21]

A mixture of compound 1 (70.00 mg, 0.16 mmol), compound 1A (40.00 mg, 0.24 mmol), K₂CO₃ (66 mg, 0.48 mmol) and Pd(dppf)Cl₂ (11 mg, 0.016 mmol) in dioxane (2.00 ML) and water (0.40 mL) was stirred at 90° C. under N₂ atmosphere for 16 hours. TLC (Petroleum ether:EtOAc=1:1) showed the starting material remained and a new spot were observed. The mixture was concentrated to get a residue, which was purified by pre-TLC (Petroleum ether:EtOAc=1:1) to get a racemic compound (30.00 mg).

The racemic compound was further purified by SFC (DAICEL CHIRALPAK AD-H (250 mm×30 mm, 5 μm), Mobile phase: A: CO₂, B: ETOH (0.1% NH₃H₂O), Flow Rate (60 ml/min) to give:

(R)-5-ethyl-8,8-dimethyl-5-(3-(pyridin-4-yl)phenyl)-5,8,9,10-tetrahydrobenzo[b][1,8]naphthyridin-6(7H)-one. (2A) (10.00 mg, 33.33% yield, 99.24% purity, Rt=1.659 min) as a white solid.

LCMS: tR=1.659 min., (ES+) M/Z (M+H)+: 410.0.

HPLC: (purity: 99.24%).

¹H NMR: (400 MHz, methanol-d4) δ=8.54-8.56 (m, 2H), 7.98 (d, J=1.6 Hz, 1H), 7.97 (s, 1H), 7.64-7.66 (m, 2H), 7.47-7.52 (m, 2H), 7.39-7.41 (m, 1H), 7.14 (d, J=5.6 Hz, 1H), 6.83-6.85 (m, 11H), 3.03-3.08 (m, 1H), 2.61 (s, 2H), 2.11-2.20 (m, 3H), 1.15 (s, 3H), 1.12 (s, 3H), 0.83 (t, J=6.8 Hz, 3H).

(S)-5-ethyl-8,8-dimethyl-5-(3-(pyridin-4-yl)phenyl)-5,8,9,10-tetrahydrobenzo[b][1,8]naphthyridin-6(7H)-one. (2B) (10.00 mg, 33.33% yield, 100% purity, Rt=1.964 min) as a white solid.

LCMS: tR=1.964 min., (ES⁺) M/Z (M+H)⁺: 410.0.

HPLC: (purity: 100%).

¹H NMR: (400 MHz, methanol-d4) δ=8.53-8.55 (m, 2H), 7.97 (d, J=3.2 Hz, 1H), 7.97 (s, 1H), 7.64-7.65 (m, 2H), 7.47-7.49 (m, 2H), 7.39-7.41 (m, 1H), 7.13 (d, J=6.4 Hz, 1H), 6.83-6.85 (m, 1H), 3.03-3.08 (m, 1H), 2.61 (s, 2H), 2.05-2.19 (m, 3H), 1.15 (s, 3H), 1.12 (s, 3H), 0.82 (t, J=7.2 Hz, 3H).

### Example 2.13. (R)-5-ethyl-8,8-dimethyl-5-(3-(pyridin-3-yl)phenyl)-5,8,9,10-tetrahydrobenzo[b][1,8]naphthyridin-6(7H)-one. (2A)

### (S)-5-ethyl-8,8-dimethyl-5-(3-(pyridin-3-yl)phenyl)-5,8,9,10-tetrahydrobenzo[b][1,8]naphthyridin-6(7H)-one. (2B)

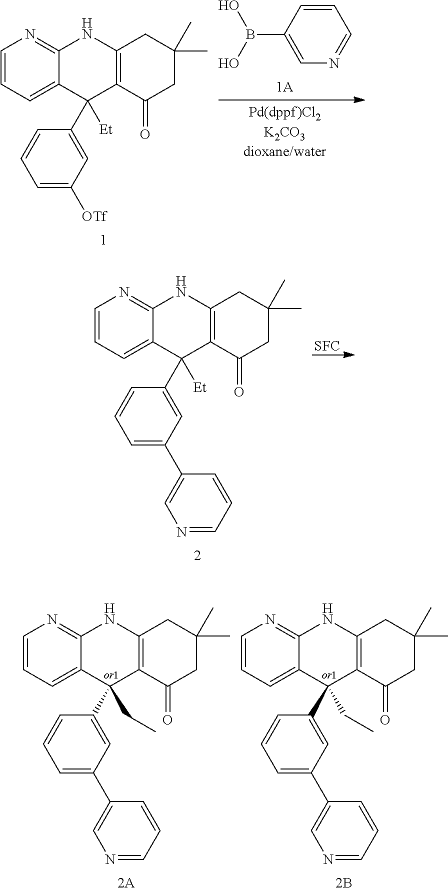

A mixture of compound 1 (100.00 mg, 0.21 mmol), pyridin-3-ylboronic acid (40.00 mg, 0.32 mmol), K₂CO₃ (87 mg, 0.63 mmol) and Pd(dppf)Cl₂ (15 mg, 0.021 mmol) in dioxane (2.00 mL) and water (0.40 mL) was stirred at 90° C. under N₂ atmosphere for 16 hours. TLC (Petroleum ether:EtOAc=1:1) showed the starting material remained and a new spot were observed. The mixture was concentrated to get a residue, which was purified by pre-TLC (Petroleum ether:EtOAc=1:1) to get a racemic compound 2 (30.00 mg).

The racemic compound was further purified by SFC (DAICEL CHIRALPAK AS (250 mm×30 mm, 10 μm), Mobile phase: A: CO₂, B: ETOH (0.1% NH₃H₂O), Flow Rate (60 ml/min) to give:

(R)-5-ethyl-8,8-dimethyl-5-(3-(pyridin-3-yl)phenyl)-5,8,9,10-tetrahydrobenzo[b][1,8]naphthyridin-6(7H)-one. (2A) (7.00 mg, 23.33% yield) as a white solid.

LCMS: tR=2.316 min., (ES+) M/Z (M+H)+: 410.1.

HPLC: (purity: 99.29%).

¹H NMR: (500 MHz, methanol-d4) δ=8.71 (d, J=2.0 Hz, 1H), 8.49 (d, J=3.5 Hz, 1H), 7.98-7.02 (m, 1H), 7.96-7.97 (m, 1H), 7.47 (s, 1H), 7.42-7.44 (m, 2H), 7.15-7.17 (m, 1H), 6.83-6.85 (m, 1H), 3.02-3.09 (m, 1H), 2.61 (s, 2H), 2.08-2.20 (s, 3H), 1.17 (s, 3H), 1.13 (s, 3H), 0.81-0.86 (m, 3H).

(S)-5-ethyl-8,8-dimethyl-5-(3-(pyridin-3-yl)phenyl)-5,8,9,10-tetrahydrobenzo[b][1,8]naphthyridin-6(7H)-one. (2B) (9.00 mg, 30.00% yield) as a white solid.

LCMS: tR=2.547 min., (ES⁺) M/Z (M+H)⁺: 410.0.

HPLC: (purity: 99.70%).

¹H NMR: (500 MHz, methanol-d4) δ=8.73 (s, 1H), 8.06 (s, 1H), 7.98 (d, J=1.5 Hz, 1H), 7.97 (s, 1H), 7.68 (s, 1H), 7.47 (s, 1H), 7.39-7.40 (m, 1H), 7.37-7.38 (m, 1H), 7.15-7.17 (m, 2H), 6.85 (d, J=5.0 Hz, 1H), 6.83 (d, J=5.0 Hz, 1H), 3.04-3.09 (m, 1H), 2.61 (s, 2H), 1.16 (s, 3H), 1.13 (s, 3H), 0.82 (t, J=7.5 Hz, 3H).

### Example 2.14. (R)-5-ethyl-8,8-dimethyl-5-(3-(3-methylpyridin-4-yl)phenyl)-5,8,9,10-tetrahydrobenzo[b][1,8]naphthyridin-6(7H)-one. (3A)

### (S)-5-ethyl-8,8-dimethyl-5-(3-(3-methylpyridin-4-yl)phenyl)-5,8,9,10-tetrahydrobenzo[b][1,8]naphthyridin-6(7H)-one. (3B)

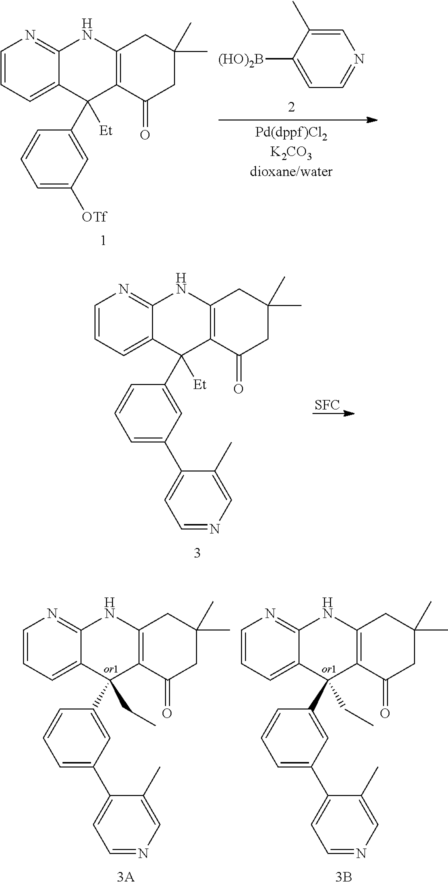

Compound 1 (70.00 mg, 145.68 μmol), compound 2 (25.93 mg, 189.38 μmol), Pd(dppf)Cl₂ (21.32 mg, 29.14 umol) and K₂CO₃ (60.40 mg, 437.04 μmol) were added into Dioxane (4.00 mL)/H₂O (0.4 mL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to 3 (15.00 mg, 24.31% yield) as white solid. LCMS: ((ES+) M/Z (M+H)+: 424.2).

Compound 3 (15 mg, 0.035 mmol) was separated by SFC (DAICEL CHIRALCEL OJ-H (250 mm×50 mm, 10 μm); 0.1% NH₃H₂O ETOH—CO₂; 40%, Flow Rate: 70 ml/min) to give:

(R)-5-ethyl-8,8-dimethyl-5-(3-(3-methylpyridin-4-yl)phenyl)-5,8,9,10-tetrahydrobenzo[b][1,8]naphthyridin-6(7H)-one. (3A) (6 mg, 40.01% yield) as a white solid.

LCMS: tR=0.311 min., (ES+) M/Z (M+H)+: 424.2.

HPLC: (purity: 100%).

¹H NMR: (400 MHz, methanol-d4) δ=8.38 (s, 1H), 8.33 (d, J=5.2 Hz, 1H), 7.94-7.96 (m, 1H), 7.46 (d, J=7.6 Hz, 1H), 7.39-7.31 (m, 2H), 7.22 (d, J=5.2 Hz, 1H), 7.15-7.05 (m, 2H), 6.81-6.84 (m, 1H), 3.07-2.95 (m, 1H), 2.63-2.50 (m, 2H), 2.18 (s, 3H), 2.15-2.00 (m, 3H), 1.17-1.03 (m, 6H), 0.78 (t, J=7.2 Hz, 3H).

(S)-5-ethyl-8,8-dimethyl-5-(3-(3-methylpyridin-4-yl)phenyl)-5,8,9,10-tetrahydrobenzo[b][1,8]naphthyridin-6(7H)-one. (3B) (6 mg, 40.01% yield) as a white solid.

LCMS: tR=0.490 min., (ES+) M/Z (M+H)+: 424.2.

HPLC: (purity: 100%).

¹H NMR: (400 MHz, methanol-d4) δ=8.38 (s, 1H), 8.33 (d, J=4.8 Hz, 1H), 7.94-7.96 (m, 1H), 7.46 (d, J=8.4 Hz, 1H), 7.39-7.31 (m, 2H), 7.22 (d, J=5.2 Hz, 1H), 7.15-7.06 (m, 2H), 6.81-6.84 (m, 1H), 3.06-2.95 (m, 1H), 2.56 (d, J=1.6 Hz, 2H), 2.19 (s, 3H), 2.15-2.00 (m, 3H), 1.17-1.03 (m, 6H), 0.78 (t, J=7.2 Hz, 3H).

### Example 2.15. (R)-5-ethyl-8,8-dimethyl-5-(3-(2-methylpyridin-4-yl)phenyl)-5,8,9,10-tetrahydrobenzo[b][1,8]naphthyridine-6(7H)-one. (3A)

### (S)-5-ethyl-8,8-dimethyl-5-(3-(2-methylpyridin-4-yl)phenyl)-5,8,9,10-tetrahydrobenzo[b][1,8]naphthyridin-6(7H)-one. (3B)

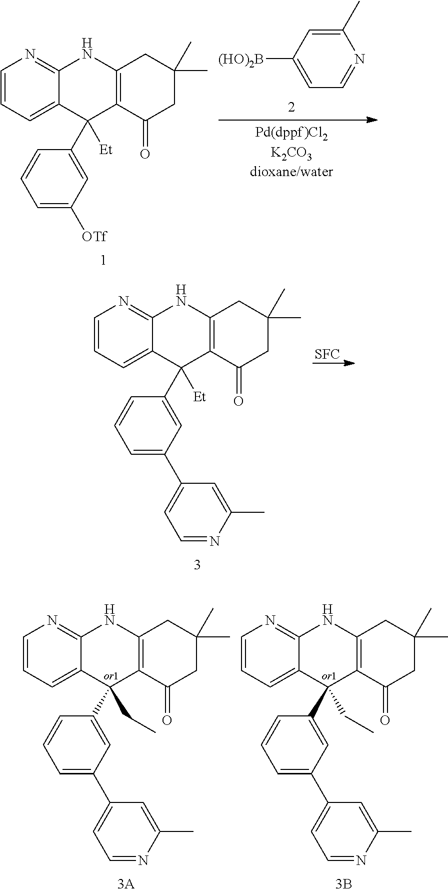

Compound 1 (70.00 mg, 145.68 μmol), compound 2 (41.68 mg, 189.38 μmol), Pd (dppf) Cl₂ (10.66 mg, 14.57 μmol) and K₂CO₃ (60.40 mg, 437.04 μmol) were added into Dioxane (4.00 mL)/H₂O (399.96 μL). The mixture was stirred at 90° C. for 16 hours under N₂ atmosphere.

LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to 3 (5.00 mg, 8.08% yield) as white solid.

LCMS: (ES⁺) M/Z (M+H)⁺=425.1.

Compound 5 (20.00 mg, 47.22 μmol) was separated by SFC (DAICEL CHIRALPAK AD (250 mm×50 mm, 10 μm); 0.1% NH₃H₂O ETOH; 40%; Flow Rate: 80 ml/min) to give:

(R)-5-ethyl-8,8-dimethyl-5-(3-(2-methylpyridin-4-yl)phenyl)-5,8,9,10-tetrahydrobenzo[b][1,8]naphthyridine-6(7H)-one. (3A) (4.00 mg, 20.00% yield) as a white solid.

LCMS: tR=0.323 min., (ES⁺) M/Z (M+H)⁺=424.2.

HPLC: (purity: 100%).

¹H NMR (500 MHz, methanol-d₄) δ=8.40 (d, J=5.5 Hz, 1H), 7.97 (d, J=1.5, 4.7 Hz, 1H), 7.74 (s, 1H), 7.52-7.41 (m, 4H), 7.39-7.35 (m, 1H), 7.14 (d, J=1.5, 7.7 Hz, 1H), 6.82 (d, J=5.0, 7.8 Hz, 1H), 3.05 (d, J=7.5, 13.0 Hz, 1H), 2.61 (s, 2H), 2.57 (s, 3H), 2.21-2.05 (m, 3H), 1.14 (d, J=13.0 Hz, 6H), 0.82 (t, J=7.5 Hz, 3H).

(S)-5-ethyl-8,8-dimethyl-5-(3-(2-methylpyridin-4-yl)phenyl)-5,8,9,10-tetrahydrobenzo[b][1,8]naphthyridin-6(7H)-one. (3B) (5.00 mg, 25.00% yield) as a white solid.

LCMS: tR=1.785 min., (ES+) M/Z (M+H)+: 424.2).

HPLC: (purity: 100%).

¹H NMR: (500 MHz, methanol-d4) δ=8.40 (d, J=5.5 Hz, 1H), 7.97 (d, J=1.5, 4.5 Hz, 1H), 7.74 (s, 1H), 7.53-7.41 (m, 4H), 7.40-7.34 (m, 1H), 7.14 (d, J=1.5, 8.0 Hz, 1H), 6.83 (d, J=4.5, 7.7 Hz, 1H), 3.09-3.00 (m, 1H), 2.61 (s, 2H), 2.57 (s, 3H), 2.21-2.05 (m, 3H), 1.14 (d, J=13.0 Hz, 6H), 0.82 (t, J=7.5 Hz, 3H).

### Example 2.16. (R)-5-ethyl-5-(3-(3-methoxypyridin-4-yl)phenyl)-8,8-dimethyl-5,8,9,10-tetrahydrobenzo[b][1,8]naphthyridin-6(7H)-one. (3A)

### (S)-5-ethyl-5(3-(3-methoxypyridin-4-yl)phenyl)-8,8-dimethyl-5,8,9,10-tetrahydrobenzo[b][1,8]naphthyridin-6(7H)-one. (3B)

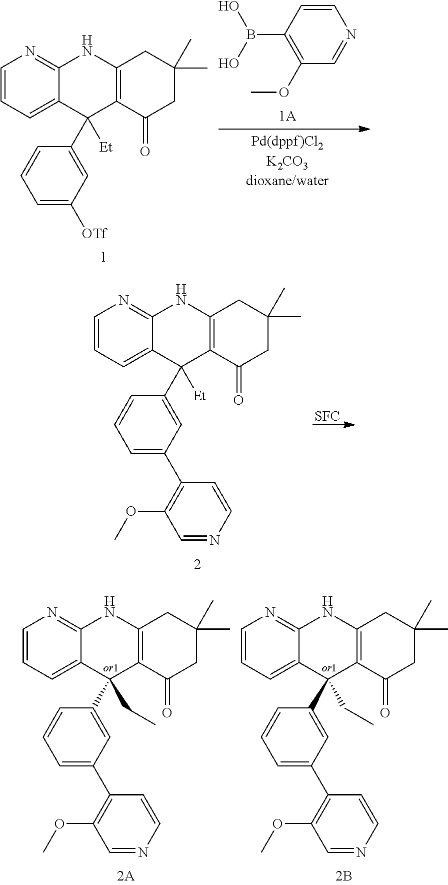

A mixture of compound 1 (100.00 mg, 0.21 mmol), compound 1A (49.00 mg, 0.32 mmol), K2CO3 (87 mg, 0.63 mmol) and Pd(dppf)Cl₂ (15 mg, 0.021 mmol) in dioxane (2.00 mL) and water (0.40 mL) was stirred at 90° C. under N₂ atmosphere for 16 hours. TLC (Petroleum ether:EtOAc=1:1) showed the starting material remained and a new spot were observed. The mixture was concentrated to get a residue, which was purified by pre-TLC (Petroleum ether:EtOAc=1:1) to get a racemic compound 2 (30.00 mg).

The racemic compound was further purified by SFC (DAICEL CHIRALPAK AD-H (250 mm×30 mm, 5 μm), Mobile phase: A: CO₂, B: ETOH (0.1% NH₃H₂O), Flow Rate (60 ml/min) to give:

(R)-5-ethyl-5-(3-(3-methoxypyridin-4-yl)phenyl)-8,8-dimethyl-5,8,9,10-tetrahydrobenzo[b][1,8]naphthyridin-6(7H)-one. (3A) (8.00 mg, 26.67% yield) as a white solid.

LCMS: tR=1.497 min., (ES⁺) M/Z (M+H)⁺: 440.1.

HPLC: (purity: 100%).

¹H NMR: (400 MHz, methanol-d4) δ=8.31 (S, 1H), 8.19 (d, J=5.2 Hz, 1H), 7.97 (d, J=3.2 Hz, 1H), 7.61 (s, 1H), 7.45 (s, 1H), 7.31-7.34 (m, 3H), 7.14 (d, J=9.2 Hz, 1H), 6.83-6.86 (m, 1H), 3.84 (s, 3H), 3.02-3.06 (m, 1H), 2.59 (s, 2H), 2.07-2.19 (m, 3H), 1.14 (s, 3H), 1.10 (s, 3H), 0.81 (t, J=7.6 Hz, 3H).

(S)-5-ethyl-5-(3-(3-methoxypyridin-4-yl)phenyl)-8,8-dimethyl-5,8,9,10-tetrahydrobenzo[b][1,8]naphthyridin-6(7H)-one. (3B) (8.00 mg, 26.67% yield) as a white solid.

LCMS: tR=1.747 min., (ES⁺) M/Z (M+H)⁺: 440.1.

HPLC: (purity: 99.42%).

¹H NMR: (400 MHz, methanol-d4) δ=8.31 (S, 1H), 8.19 (d, J=5.2 Hz, 1H), 7.97 (d, J=3.2 Hz, 1H), 7.61 (s, 1H), 7.46 (s, 1H), 7.32-7.34 (m, 3H), 7.15 (d, J=9.6 Hz, 1H), 6.83-6.87 (m, 1H), 3.85 (s, 3H), 3.03-3.08 (m, 1H), 2.59 (s, 2H), 2.08-2.15 (m, 3H), 1.15 (s, 3H), 1.11 (s, 3H), 0.81 (t, J=7.2 Hz, 3H).

### Example 2.17. (R)-5-ethyl-8,8-dimethyl-5-(3-(6-methylpyridazin-4-yl)phenyl)-5,8,9,10-tetrahydrobenzo[b][1,8]naphthyridin-6(7H)-one. (2A)

### (S)-5-ethyl-8,8-dimethyl-5-(3-(6-methylpyridazin-4-yl)phenyl)-5,8,9,10-tetrahydrobenzo[b][1,8]naphthyridin-6(7H)-one. (2B)

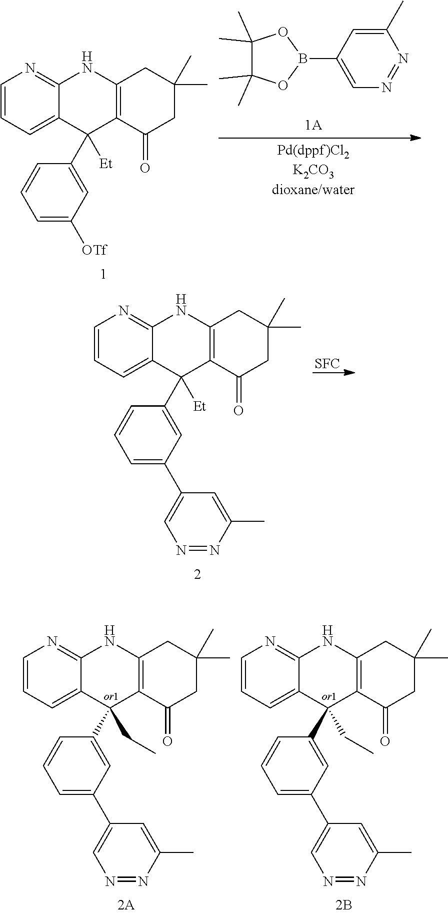

A mixture of compound 1 (50.00 mg, 0.11 mmol), compound 1A (36.00 mg, 0.23 mmol), K₂CO₃ (52 mg, 0.33 mmol) and Pd(dppf)Cl₂ (8 mg, 0.0011 mmol) in dioxane (2.00 mL) and water (0.40 mL) was stirred at 90° C. under N₂ atmosphere for 16 hours. TLC (Petroleum ether:EtOAc=1:1) showed the starting material remained and a new spot were observed. The mixture was concentrated to get a residue, which was purified by pre-TLC (Petroleum ether:EtOAc=1:1) to get a racemic compound (30.00 mg).

The racemic compound was further purified by SFC (DAICEL CHIRALPAK AS (250 mm×30 mm, 10 μm), Mobile phase: A: CO₂, B: ETOH (0.1% NH₃H₂O), Flow Rate (60 ml/min) to give:

(R)-5-ethyl-8,8-dimethyl-5-(3-(6-methylpyridazin-4-yl)phenyl)-5,8,9,10-tetrahydrobenzo[b][1,8]naphthyridin-6(7H)-one. (2A) (11.00 mg, 36.67% yield) as a white solid.

LCMS: tR=2.939 min., (ES⁺) M/Z (M+H)⁺: 425.1.

HPLC: (purity: 99.29%).

¹H NMR: (400 MHz, methanol-d4) δ=9.30 (s, 1H), 7.98 (d, J=3.2 Hz, 1H), 7.86 (s, 2H), 7.53-7.57 (m, 2H), 7.41-7.45 (m, 1H), 7.14 (d, J=6.8 Hz, 1H), 6.81-6.85 (m, 1H), 3.03-3.06 (m, 1H), 2.74 (s, 3H), 2.57-2.67 (m, 2H), 2.07-2.19 (m, 3H), 1.15 (s, 3H), 1.12 (s, 3H), 0.83 (t, J=7.2 Hz, 3H).

(S)-5-ethyl-8,8-dimethyl-5-(3-(6-methylpyridazin-4-yl)phenyl)-5,8,9,10-tetrahydrobenzo[b][1,8]naphthyridin-6(7H)-one. (2B) (10.00 mg, 33.33% yield) as a white solid.

LCMS: tR=3.673 min., (ES⁺) M/Z (M+H)⁺: 425.1.

HPLC: (purity: 100%).

¹H NMR: (400 MHz, methanol-d4) δ=9.30 (s, 1H), 7.98 (d, J=4.8 Hz, 1H), 7.86 (s, 2H), 7.54-7.57 (m, 2H), 7.42-7.46 (m, 1H), 7.14-7.16 (m, 1H), 6.82-6.85 (m, 1H), 3.02-3.07 (m, 1H), 2.74 (s, 3H), 2.58-2.62 (m, 2H), 2.08-2.20 (m, 3H), 1.16 (s, 3H), 1.13 (s, 3H), 0.83 (t, J=7.2 Hz, 3H).

### Example 2.18. (R)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3A)

### (S)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3B)

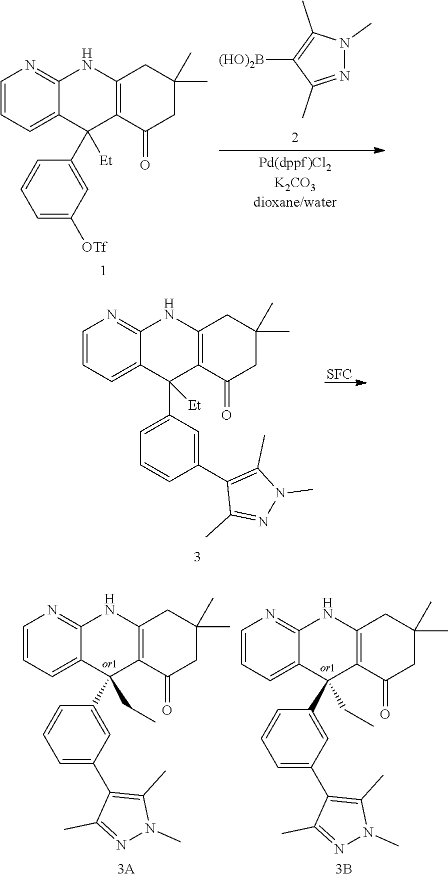

Compound 1 (70.00 mg, 145.68 μmol), compound 2 (44.72 mg, 189.39 μmol), Pd(dppf)Cl₂ (10.66 mg, 14.57 μmol) were added into Dioxane (4.00 mL)/H₂O (399.96 μL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to 3 (15.00 mg, 23.37% yield) as white solid.

LCMS: ((ES⁺) M/Z (M+H)⁺: 441.2).

3 (50 mg, 0.11 mmol) was separated by SFC (DAICEL CHIRALPAK AD (250 mm×30 mm, 10 μm); 0.1% NH₃H₂O ETOH—CO₂; 30%, Flow Rate: 60 ml/min) to give:

(R)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3A) (17 mg, 34.00% yield).

LCMS: tR=1.290 min., (ES⁺) M/Z (M+H)⁺: 441.2.

HPLC: (purity: 100%).

¹H NMR: (400 MHz, methanol-d4) δ=7.95-7.97 (m, 1H), 7.36-7.31 (m, 1H), 7.31-7.25 (m, 1H), 7.23 (s, 1H), 7.12-7.15 (m, 1H), 6.98 (d, J=7.2 Hz, 1H), 6.82-6.85 (m, 1H), 3.73 (s, 3H), 3.11-3.00 (m, 1H), 2.64-2.51 (m, 2H), 2.21-2.01 (m, 9H), 1.18-1.05 (m, 6H), 0.80 (t, J=7.6 Hz, 3H).

(S)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3B) (22 mg, 44.00% yield) both as white solid.

LCMS: tR=1.517 min., (ES⁺) M/Z (M+H)⁺: 441.2.

HPLC: (purity: 100%).

¹H NMR: (400 MHz, methanol-d4) δ=7.95-7.97 (m, 1H), 7.36-7.31 (m, 1H), 7.30-7.25 (m, 1H), 7.23 (t, J=1.6 Hz, 1H), 7.12-7.14 (m, 1H), 6.96-6.99 (m, 1H), 6.82-6.85 (m, 1H), 3.11-3.00 (m, 1H), 2.63-2.51 (m, 2H), 2.20-2.00 (m, 9H), 1.17-1.07 (m, 6H), 0.80 (t, J=7.6 Hz, 3H).

### Example 2.19. (R)-5-ethyl-8,8-dimethyl-5-(3-(1-methyl-1H-pyrazol-5-yl)phenyl)-5,8,9,10-tetrahydrobenzo[b][1,8]naphthyridin-6(7H)-one. (3A)

### (S)-5-ethyl-8,8-dimethyl-5-(3-(1-methyl-1H-pyrazol-5-yl)phenyl)-5,8,9,10-tetrahydrobenzo[b][1,8]naphthyridin-6(7H)-one. (3B)

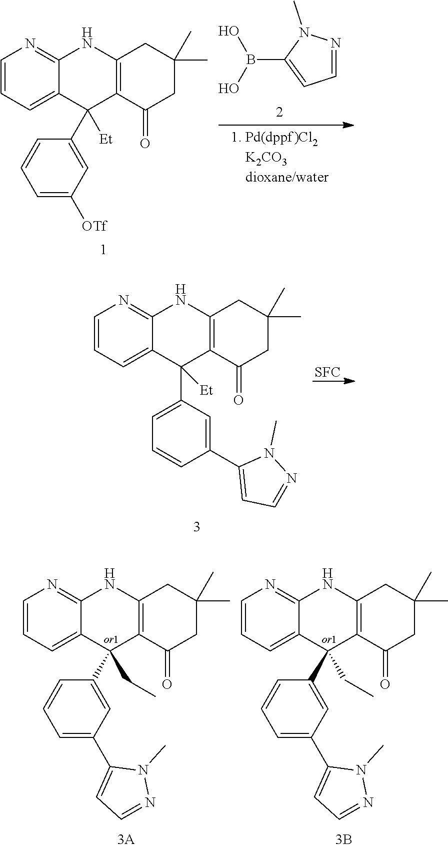

A mixture of compound 1 (70.00 mg, 0.16 mmol), compound 2 (30.00 mg, 0.24 mmol), K₂CO₃ (66 mg, 0.48 mmol) and Pd(dppf)Cl₂ (11 mg, 0.016 mmol) in dioxane (2.00 mL) and water (0.40 mL) was stirred at 90° C. under N₂ atmosphere for 16 hours. TLC (Petroleum ether:EtOAc=1:1) showed the starting material remained and a new spot were observed. The mixture was concentrated to get a residue, which was purified by pre-TLC (Petroleum ether:EtOAc=1:1) to get the racemic compound (40.00 mg).

The racemic compound was further purified by SFC (REGIS (s, s) WHELK-O1 (250 mm×30 mm, 5 μm), Mobile phase: A: CO₂, B: ETOH (0.1% NH₃H₂O), Flow Rate (70 ml/min) to give:

(R)-5-ethyl-8,8-dimethyl-5-(3-(1-methyl-1H-pyrazol-5-yl)phenyl)-5,8,9,10-tetrahydrobenzo[b][1,8]naphthyridin-6(7H)-one. (3A) (18.00 mg, 45.00% yield, 99.24% purity) as a white solid.

LCMS: tR=1.402 min., (ES⁺) M/Z (M+H)⁺: 413.2.

HPLC: (purity: 100%).

¹H NMR: (400 MHz, methanol-d4) δ=7.98 (d, J=1.6 Hz, 1H), 7.47-7.49 (m, 3H), 7.45-7.46 (m, 1H), 7.33-7.36 (m, 1H), 7.12-7.21 (m, 1H), 6.83-6.84 (m, 1H), 6.30-6.31 (m, 1H), 3.79 (s, 3H), 3.00-3.05 (m, 1H), 2.59 (s, 2H), 2.08-2.19 (m, 3H), 1.14 (s, 3H), 1.10 (s, 3H), 0.80 (t, J=7.2 Hz, 3H).

(S)-5-ethyl-8,8-dimethyl-5-(3-(1-methyl-1H-pyrazol-5-yl)phenyl)-5,8,9,10-tetrahydrobenzo[b][1,8]naphthyridin-6(7H)-one. (3B) (10.00 mg, 25.00% yield, 100% purity, Rt=1.834 min) as a white solid.

LCMS: tR=1.834 min., (ES⁺) M/Z (M+H)⁺: 413.1.

HPLC: (purity: 100%).

¹H NMR: (400 MHz, methanol-d4) δ=7.98 (d, J=1.6 Hz, 1H), 7.47-7.49 (m, 3H), 7.44-7.46 (m, 1H), 7.33-7.36 (m, 1H), 7.12-7.21 (m, 1H), 6.82-6.85 (m, 1H), 6.30-6.31 (m, 1H), 3.79 (s, 3H), 3.00-3.05 (m, 1H), 2.59 (s, 2H), 2.04-2.14 (m, 3H), 1.14 (s, 3H), 1.10 (s, 3H), 0.81 (t, J=7.2 Hz, 3H).

### Example 2.20. (R)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3A)

### (S)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3B)

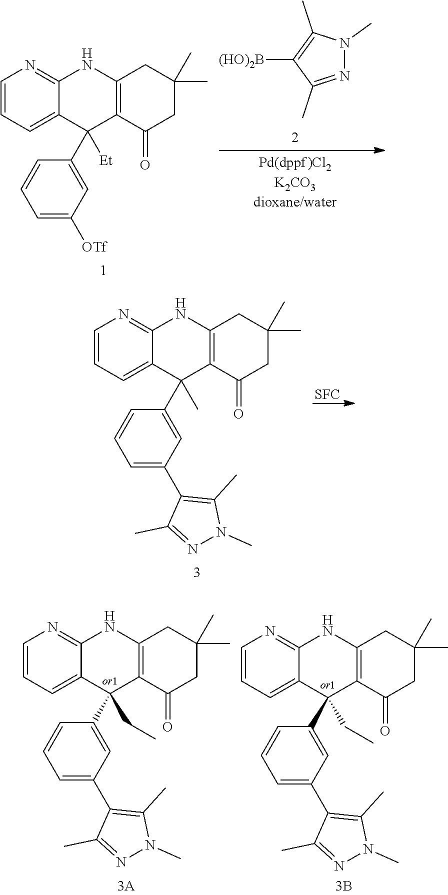

Compound 1 (70.00 mg, 145.68 μmol), compound 2 (42.06 mg, 189.38 μmol), Pd (dppf)Cl₂ (10.66 mg, 14.57 μmol) and K₂CO₃ (60.40 mg, 437.04 μmol) were added into Dioxane (4.00 mL)/H₂O (0.4 mL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to 3 (15.00 mg, 24.14% yield) as white solid.

LCMS: (ES⁺) M/Z (M+H)⁺: 427.1.

Racemic 3 (15.00 mg) was separated by SFC (DAICEL CHIRALPAK AS (250 mm×30 mm, 10 μm); Neu-ETOH; 20%; Flow Rate: 60 ml/min) to give:

(R)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3A) (6.00 mg, 40.00% yield, 100% purity, Rt=2.133 min) as a white solid

LCMS: tR=2.133 min., (ES⁺) M/Z (M+H)⁺: 427.1.

HPLC: (purity: 100%).

¹H NMR: (500 MHz, methanol-d4) δ=8.22 (s, 1H), 8.18 (s, 1H), 7.97 (M, 1H), 7.51 (d, J=7.0 Hz, 1H), 7.43 (s, 1H), 7.35 (t, J=7.5 Hz, 1H), 7.20-7.11 (m, 2H), 6.85 (m, 1H), 3.83 (s, 3H), 3.06-2.98 (m, 1H), 2.58 (s, 2H), 2.18-2.04 (m, 3H), 1.14 (s, 3H), 1.10 (s, 3H), 0.80 (t, J=7.5 Hz, 3H).

(S)-5-ethyl-8,8-dimethyl-5-(3-(1,3,5-trimethyl-1H-pyrazol-4-yl)phenyl)-5,8,9,10-tetrahydrobenzo[b][1,8]naphthyridin-6(7H)-one. (3B) (7.00 mg, 46.66% yield, 100% purity, Rt=1.326 min) as a white solid.

LCMS: tR=2.325 min., (ES⁺) M/Z (M+H)⁺: 427.1.

HPLC: (purity: 100%).

¹H NMR: (500 MHz, methanol-d4) δ=8.22 (s, 1H), 8.18 (s, 1H), 7.97 (d, J=3.0 Hz, 1H), 7.51 (m, 1H), 7.43 (s, 1H), 7.35 (t, J=7.5 Hz, 1H), 7.20-7.12 (m, 2H), 6.85 (m, 1H), 3.83 (s, 3H), 3.02 (m, 1H), 2.58 (s, 2H), 2.19-2.06 (m, 3H), 1.14 (s, 3H), 1.10 (s, 3H), 0.80 (t, J=7.5 Hz, 3H).

### Example 2.21. (R)-5-ethyl-8,8-dimethyl-5-(3-(1-methyl-1H-pyrazol-4-yl)phenyl)-5,8,9,10-tetrahydrobenzo[b][1,8]naphthyridin-6(7H)-one. (2A)

### (S)-5-ethyl-8,8-dimethyl-5-(3-(1-methyl-1H-pyrazol-4-yl)phenyl)-5,8,9,10-tetrahydrobenzo[b][1,8]naphthyridin-6(7H)-one. (2B)

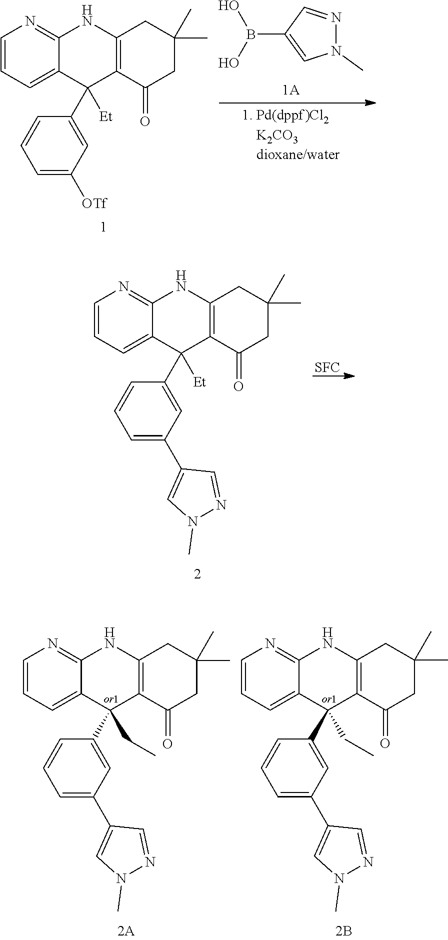

Compound 1 (70.00 mg, 145.68 μmol), compound 1A (42.06 mg, 189.38 μmol), Pd(dppf)Cl₂ (10.66 mg, 14.57 μmol) and K₂CO₃ (60.40 mg, 437.04 μmol) were added into Dioxane (4.00 mL)/H₂O (400 μL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to 2 (15.00 mg, 24.14% yield) as white solid. LCMS: ((ES+) M/Z (M+H)+: 427.1).

Racemic 2 (15.00 mg, 35.17 μmol) was separated by SFC (DAICEL CHIRALPAK AD (250 mm×50 mm, 10 μm); 0.1% NH₃H₂O ETOH; 40%; Flow Rate: 70 ml/min) to give:

(R)-5-ethyl-8,8-dimethyl-5-(3-(1-methyl-1H-pyrazol-4-yl)phenyl)-5,8,9,10-tetrahydrobenzo[b][1,8]naphthyridin-6(7H)-one. (2A) (5.00 mg, 33.33% yield) as a white solid.

LCMS: tR=0.283 min., (ES+) M/Z (M+H)+: 427.2.

HPLC: (purity: 100%).

¹H NMR: (500 MHz, methanol-d4) δ=8.22 (s, 1H), 8.18 (s, 1H), 7.97 (m, 1H), 7.51 (d, J=7.0 Hz, 1H), 7.43 (s, 1H), 7.35 (t, J=7.5 Hz, 1H), 7.20-7.11 (m, 2H), 6.85 (m, 1H), 3.83 (s, 3H), 3.06-2.98 (m, 1H), 2.58 (s, 2H), 2.18-2.04 (m, 3H), 1.14 (s, 3H), 1.10 (s, 3H), 0.80 (t, J=7.5 Hz, 3H).

(S)-5-ethyl-8,8-dimethyl-5-(3-(1-methyl-1H-pyrazol-4-yl)phenyl)-5,8,9,10-tetrahydrobenzo[b][1,8]naphthyridin-6(7H)-one (5.00 mg, 33.33% yield) as a white solid.

LCMS: tR=0.469 min., (ES⁺) M/Z (M+H)⁺: 427.2.

HPLC: (purity: 100%).

¹H NMR: (500 MHz, methanol-d4) δ=7.97 (d, J=1.5, 4.9 Hz, 1H), 7.48-7.41 (m, 2H), 7.34 (d, J=7.5 Hz, 1H), 7.17 (d, J=7.6 Hz, 1H), 7.12 (d, J=1.5, 7.8 Hz, 1H), 6.83 (d, J=5.0, 7.8 Hz, 1H), 6.08 (s, 1H), 3.70 (s, 3H), 3.02 (d, J=7.5, 13.0 Hz, 1H), 2.59 (d, J=2.5 Hz, 2H), 2.23 (s, 3H), 2.19-2.05 (m, 3H), 1.16-1.08 (m, 6H), 0.80 (t, J=7.5 Hz, 3H).

### Example 2.22. (R)-5-(3-(2,5-dimethylpyridin-4-yl)phenyl)-5-ethyl-8,8-dimethyl-5,8,9,10-tetrahydrobenzo[b][1,8]naphthyridin-6(7H)-one. (4A)

### (S)-5-(3-(2,5-dimethylpyridin-4-yl)phenyl)-5-ethyl-8,8-dimethyl-5,8,9,10-tetrahydrobenzo[b][1,8]naphthyridin-6(7H)-one. (4B)

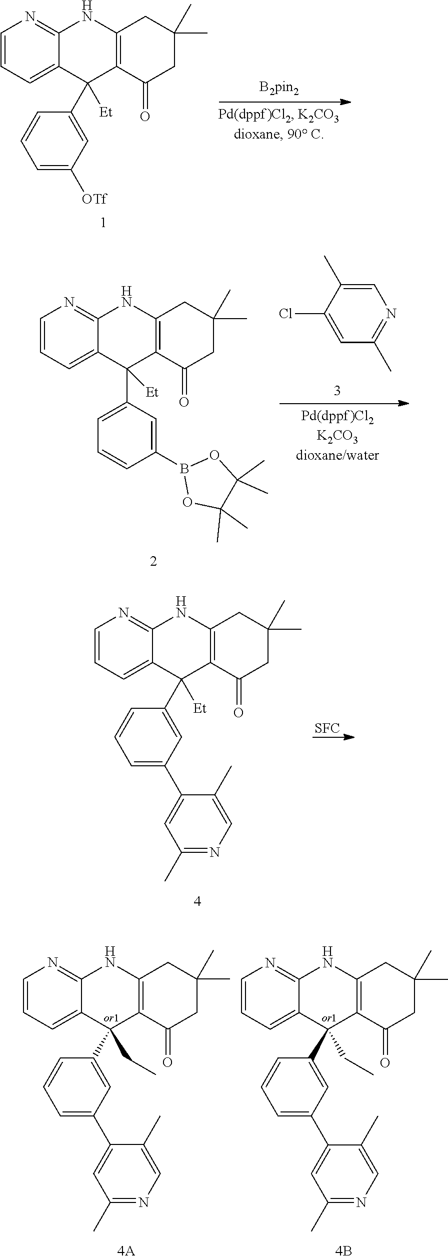

### Step 1: 5-ethyl-8,8-dimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one. (2)

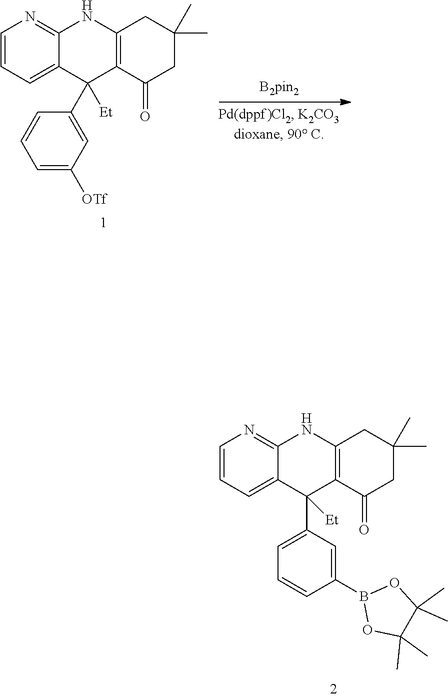 [SMILES: CCC1(c2cccc(OS(=O)(=O)C(F)(F)F)c2)C2=C(CC(C)(C)CC2=O)Nc2ncccc21 ; CCC1(c2cccc(B3OC(C)(C)C(C)(C)O3)c2)C2=C(CC(C)(C)CC2=O)Nc2ncccc21]

A mixture of compound 1 (100.00 mg, 0.21 mmol), B₂pin₂ (79.27 mg, 0.31 mmol), K₂CO₃ (86.29 mg, 0.62 μmol) and Pd(dppf)Cl₂ (15.23 mg, 0.021 mmol) in dioxane (10.0 mL) was stirred at 90° C. for 14 hours under N₂. TLC (petroleum ether/EtOAc=3/1) showed reactant 1 was consumed completely and a new spot was detected. The mixture was concentrated to give the residue, which was purified by silica gel (petroleum ether/EtOAc=10/1 to 3/1) to get compound 2 (70.00 mg, 73.5% yield) as a yellow oil.

### Step 2: (R)-5-(3-(2,5-dimethylpyridin-4-yl)phenyl)-5-ethyl-8,8-dimethyl-5,8,9,10-tetrahydrobenzo[b][1,8]naphthyridin-6(7H)-one. (4A)

### (S)-5-(3-(2,5-dimethylpyridin-4-yl)phenyl)-5-ethyl-8,8-dimethyl-5,8,9,10-tetrahydrobenzo[b][1,8]naphthyridin-6(7H)-one. (4B)

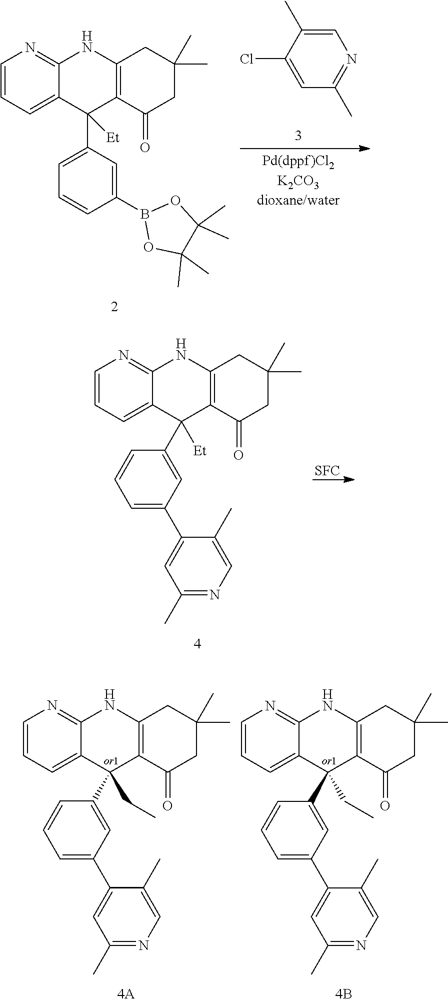

To a mixture of compound 2 (70.00 mg, 0.15 mmol), compound 3 (32.44 mg, 0.23 mmol), in dioxane (2.0 mL) and water (39.98 μL) were added K₂CO₃ (42.21 mg, 0.30 mmol) and Pd(dppf)Cl₂ (11.17 mg, 0.015 mmol). The mixture was stirred at 90° C. under N₂ atmosphere for 16 hours. TLC (petroleum ether/EtOAc=1/1) showed the starting material remained and a new spot were observed. The mixture was concentrated, and the residue was purified by prep-TLC (petroleum ether/EtOAc=1/1) to get a racemic compound 4 (30.0 mg, 43% yield.

The racemic compound 4 (30.0 mg, 43% yield) was separated by SFC (DAICEL CHIRALPAK AS (250 mm×30 mm, 10 μm), Mobile phase: A: CO₂, B: EtOH (0.1% NH₃H₂O), Flow Rate (60 mL/min), Flow Rate (60 mL/min) to give:

(R)-5-(3-(2,5-dimethylpyridin-4-yl)phenyl)-5-ethyl-8,8-dimethyl-5,8,9,10-tetrahydrobenzo[b][1,8]naphthyridin-6(7H)-one. (4A) (8.0 mg, 11.57% yield, 96.64% purity) as a white solid.

LCMS: (ES⁺) M/Z (M+H)⁺=438.1.

HPLC: (purity: 96.64%).

¹H NMR: (400 MHz, methanol-d4) δ ppm: 8.26 (s, 1H), 7.97 (d, J=2.0 Hz, 1H), 7.47 (d, J=2.0 Hz), 7.32-7.37 (m, 2H), 7.08-7.17 (m, 3H), 6.82-6.85 (m, 1H), 3.00-3.05 (m, 1H), 2.58 (s, 2H), 2.50 (s, 2H), 2.14 (s, 3H), 2.00-2.12 (m, 3H), 1.14 (s, 3H), 1.10 (s, 3H), 0.80 (t, J=8.0 Hz, 3H).

(S)-5-(3-(2,5-dimethylpyridin-4-yl)phenyl)-5-ethyl-8,8-dimethyl-5,8,9,10-tetrahydrobenzo[b][1,8]naphthyridin-6(7H)-one. (4B) (8.0 mg, 11.97% yield, 100% purity) both as a white solid.

LCMS: (ES⁺) M/Z (M+H)⁺=438.1.

HPLC: (purity: 100.0%).

¹H NMR: (400 MHz, methanol-d4) 5 ppm: 8.26 (s, 1H), 7.97 (d, J=2.0 Hz, 1H), 7.47 (d, J=2.0 Hz), 7.32-7.37 (m, 2H), 7.08-7.17 (m, 3H), 6.82-6.85 (m, 1H), 3.00-3.05 (m, 1H), 2.58 (s, 2H), 2.50 (s, 2H), 2.14 (s, 3H), 2.00-2.12 (m, 3H), 1.14 (s, 3H), 1.10 (s, 3H), 0.80 (t, J=8.0 Hz, 3H).

### Example 2.23. (R)-5-ethyl-5-(3-(3-fluoro-5-methoxypyridin-4-yl)phenyl)-8,8-dimethyl-5,8,9,10-tetrahydrobenzo[b][1,8]naphthyridin-6(7H)-one. (3A)

### (S)-5-ethyl-5-(3-(3-fluoro-5-methoxypyridin-4-yl)phenyl)-8,8-dimethyl-5,8,9,10-tetrahydrobenzo[b][1,8]naphthyridin-6(7H)-one. (3B)

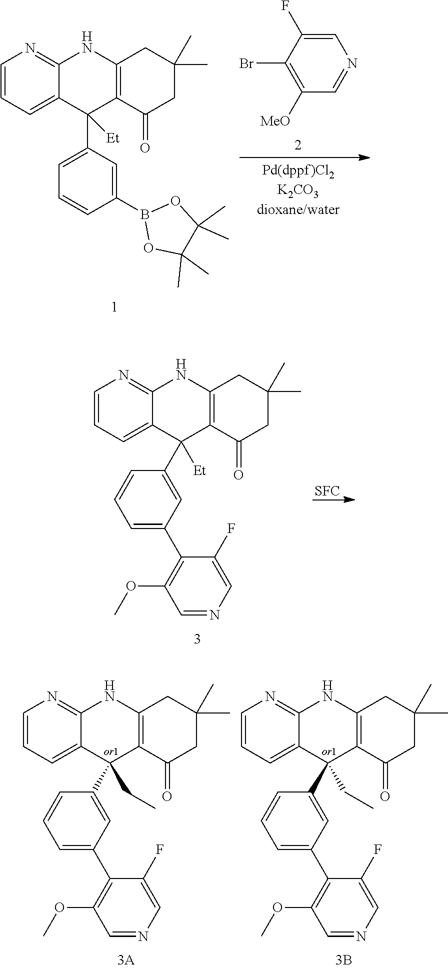

Compound 1 (70.00 mg, 145.68 μmol), compound 2 (39.02 mg, 189.38 μmol), Pd(dppf)Cl₂ (21.32 mg, 29.14 μmol) were added into Dioxane (4.00 mL)/H₂O (0.4 ML). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to give racemic 3 (10.00 mg, 15.00% yield) as white solid.

LCMS: ((ES+) M/Z (M+H)+: 458.1).

Racemic 3 (10.00 mg) was separated by SFC (DAICEL CHIRALPAK AD (250 mm×50 mm, 10 μm); 0.1% NH₃H₂O ETOH; 40%; Flow Rate: 70 ml/min) to give:

(R)-5-ethyl-5-(3-(3-fluoro-5-methoxypyridin-4-yl)phenyl)-8,8-dimethyl-5,8,9,10-tetrahydrobenzo[b][1,8]naphthyridin-6(7H)-one. (3A) (4.00 mg, 39.99% yield) as a white solid.

LCMS: tR=1.326 min., (ES⁺) M/Z (M+H)⁺: 458.1.

HPLC: (purity: 100%).

¹H NMR: (500 MHz, methanol-d4) δ=8.22 (s, 1H), 8.18 (s, 1H), 7.97 (m, 1H), 7.51 (d, J=7.0 Hz, 1H), 7.43 (s, 1H), 7.35 (t, J=7.5 Hz, 1H), 7.20-7.11 (m, 2H), 6.85 (m, 1H), 3.83 (s, 3H), 3.06-2.98 (m, 1H), 2.58 (s, 2H), 2.18-2.04 (m, 3H), 1.14 (s, 3H), 1.10 (s, 3H), 0.80 (t, J=7.5 Hz, 3H).

(S)-5-ethyl-5-(3-(3-fluoro-5-methoxypyridin-4-yl)phenyl)-8,8-dimethyl-5,8,9,10-tetrahydrobenzo[b][1,8]naphthyridin-6(7H)-one. (3B) (4.00 mg, 39.99% yield, 100% purity, Rt=1.785 min) as a white solid.

LCMS: tR=1.787 min., (ES⁺) M/Z (M+H)⁺: 458.1.

HPLC: (purity: 100%).

¹H NMR: (500 MHz, methanol-d4) δ=8.22 (s, 1H), 8.18 (s, 1H), 7.97 (d, J=3.5 Hz, 1H), 7.51 (m, 1H), 7.43 (s, 1H), 7.35 (t, J=7.5 Hz, 1H), 7.20-7.12 (m, 2H), 6.85 (m, 1H), 3.83 (s, 3H), 3.02 (m, 1H), 2.58 (s, 2H), 2.19-2.06 (m, 3H), 1.14 (s, 3H), 1.10 (s, 3H), 0.80 (t, J=7.5 Hz, 3H).

### Example 2.24. (R)-5-(3-(2,3-dimethylpyridin-4-yl)phenyl)-5-ethyl-8,8-dimethyl-5,8,9,10-tetrahydrobenzo[b][1,8]naphthyridin-6(7H)-one. (3A)

### (S)-5-(3-(2,3-dimethylpyridin-4-yl)phenyl)-5-ethyl-8,8-dimethyl-5,8,9,10-tetrahydrobenzo[b][1,8]naphthyridin-6(7H)-one. (3B)

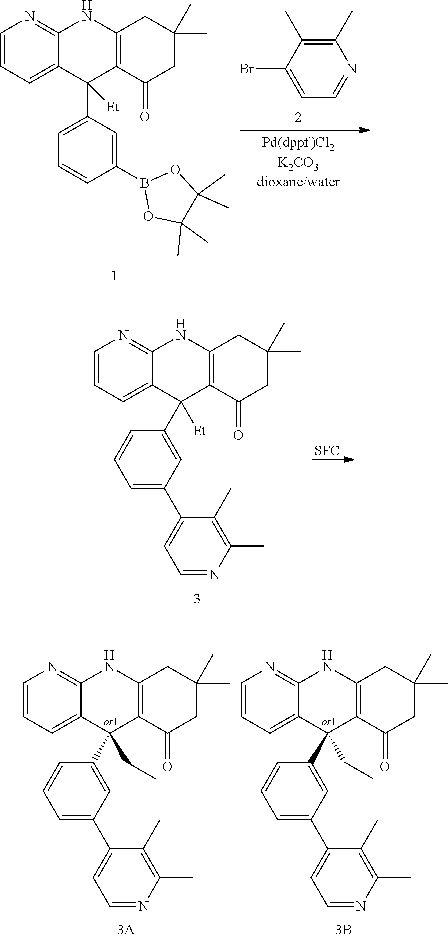

Compound 1 (40.00 mg, 87.26 μmol), compound 2 (21.11 mg, 113.44 μmol), Pd(dppf)Cl₂ (12.77 mg, 17.45 μmol) and K₂CO₃ (36.18 mg, 261.78 μmol) were added into Dioxane (4.00 mL)/H₂O (499.94 μL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to 3 (20.00 mg, 39.29% yield) as white solid. LCMS: (ES⁺) M/Z (M+H)⁺: 438.1.

Racemic 3 (20.00 mg) was separated by SFC (DAICEL CHIRALPAK AD (250 mm×30 mm, 5 μm); 0.1% NH₃H₂O IPA; 30%; Flow Rate: 60 ml/min) to give:

(R)-5-(3-(2,3-dimethylpyridin-4-yl)phenyl)-5-ethyl-8,8-dimethyl-5,8,9,10-tetrahydrobenzo[b][1,8]naphthyridin-6(7H)-one. (3A) (9.00 mg, 45.00% yield) as a white solid.

LCMS: tR=1.608 min., (ES⁺) M/Z (M+H)⁺: 438.2.

HPLC: (purity: 100%).

¹H NMR: (500 MHz, methanol-d4) δ=8.20 (d, J=5.5 Hz, 1H), 7.97 (d, J=1.5, 5.0 Hz, 1H), 7.47 (d, J=8.5 Hz, 1H), 7.38-7.29 (m, 2H), 7.13 (d, J=1.5, 7.5 Hz, 1H), 7.09-7.02 (m, 2H), 6.84 (d, J=5.0, 7.5 Hz, 1H), 3.01 (d, J=7.5, 13.0 Hz, 1H), 2.58 (d, J=2.5 Hz, 2H), 2.53 (s, 3H), 2.20-2.06 (m, 6H), 1.15-1.06 (m, 6H), 0.80 (t, J=7.5 Hz, 3H).

(S)-5-(3-(2,3-dimethylpyridin-4-yl)phenyl)-5-ethyl-8,8-dimethyl-5,8,9,10-tetrahydrobenzo[b][1,8]naphthyridin-6(7H)-one. (3B) (10.00 mg, 50.00% yield) as a white solid.

LCMS: tR=1.738 min., (ES⁺) M/Z (M+H)⁺: 438.2.

HPLC: (purity: 100%).

¹H NMR: (500 MHz, methanol-d4) δ=8.20 (d, J=5.0 Hz, 1H), 7.97 (d, J=1.5, 5.0 Hz, 1H), 7.47 (d, J=8.4 Hz, 1H), 7.39-7.28 (m, 2H), 7.13 (d, J=1.5, 7.5 Hz, 1H), 7.08-7.02 (m, 2H), 6.84 (d, J=5.0, 7.5 Hz, 1H), 3.01 (d, J=7.5, 13.0 Hz, 1H), 2.58 (d, J=2.5 Hz, 2H), 2.53 (s, 3H), 2.12 (s, 6H), 1.14-1.06 (m, 6H), 0.80 (t, J=7.5 Hz, 3H).

### Example 2.25. (R)-5-(3-(6,7-dihydro-5H-cyclopenta[b]pyridin-4-yl)phenyl)-5-ethyl-8,8-dimethyl-5,8,9,10-tetrahydrobenzo[b][1,8]naphthyridin-6(7H)-one. (3A)

### (S)-5-(3-(6,7-dihydro-5H-cyclopenta[b]pyridin-4-yl)phenyl)-5-ethyl-8,8-dimethyl-5,8,9,10-tetrahydrobenzo[b][1,8]naphthyridin-6(7H)-one. (3B)

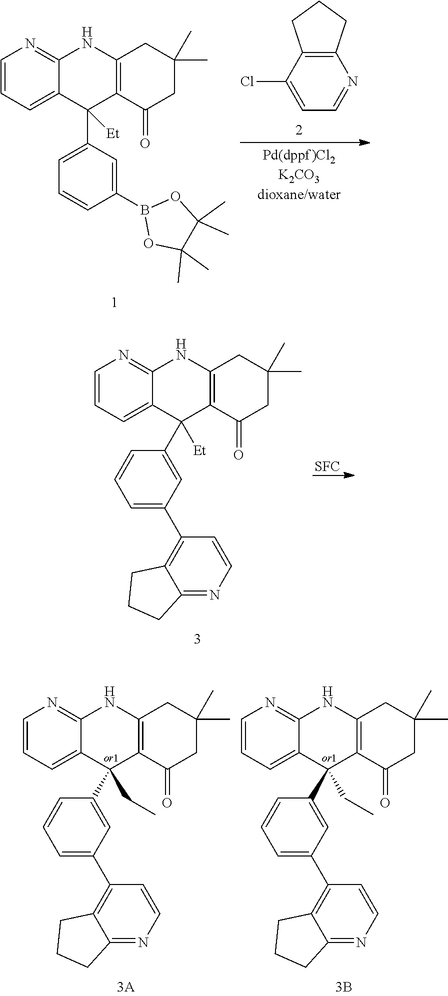

Compound 1 (40.00 mg, 87.26 μmol), compound 2 (17.43 mg, 113.44 μmol), Pd(dppf)Cl₂ (12.77 mg, 17.45 μmol) and K₂CO₃ (36.18 mg, 261.78 μmol) were added into Dioxane (4.00 mL)/H₂O (500 μL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to racemic 3 (25.00 mg, 55.61 μmol, 63.72% yield) as white solid.

LCMS: ((ES+) M/Z (M+H)+: 450.1).

Racemic 3 (20.00 mg, 44.48 μmol) was separated by SFC (DAICEL CHIRALPAK AD (250 mm×30 mm, 10 μm); 0.1% NH₃H₂O ETOH; 50%; Flow Rate: 80 ml/min) to give:

(R)-5-(3-(6,7-dihydro-5H-cyclopenta[b]pyridin-4-yl)phenyl)-5-ethyl-8,8-dimethyl-5,8,9,10-tetrahydrobenzo[b][1,8]naphthyridin-6(7H)-one. (3A). (4.00 mg, 20.00% yield) as a white solid.

LCMS: tR=1.495 min., (ES⁺) M/Z (M+H)⁺: 450.2.

HPLC: (purity: 100%).

¹H NMR: (500 MHz, MeOD-d4) δ=8.27 (d, J=5.5 Hz, 1H), 7.98 (d, J=1.5, 5.0 Hz, 1H), 7.56 (s, 1H), 7.47 (d, J=8.0 Hz, 11H), 7.36 (t, J=7.5 Hz, 1H), 7.27 (d, J=7.5 Hz, 1H), 7.19 (d, J=5.5 Hz, 1H), 7.13 (d, J=1.5, 8.0 Hz, 1H), 6.84 (d, J=5.0, 7.5 Hz, 1H), 3.10-2.93 (m, 5H), 2.59 (d, J=4.5 Hz, 2H), 2.20-2.04 (m, 5H), 1.17-1.07 (m, 6H), 0.81 (t, J=7.5 Hz, 3H).

(S)-5-(3-(6,7-dihydro-5H-cyclopenta[b]pyridin-4-yl)phenyl)-5-ethyl-8,8-dimethyl-5,8,9,10-tetrahydrobenzo[b][1,8]naphthyridin-6(7H)-one. (3B) (8.00 mg, 40.00% yield) as a white solid.

LCMS: tR=2.144 min., (ES⁺) M/Z (M+H)⁺: 450.2.

¹H NMR: (500 MHz, methanol-d4) δ=8.27 (d, J=5.5 Hz, 1H), 7.98 (d, J=1.5, 5.0 Hz, 11H), 7.56 (s, 1H), 7.47 (d, J=8.0 Hz, 1H), 7.36 (t, J=7.5 Hz, 1H), 7.27 (d, J=7.5 Hz, 11H), 7.19 (d, J=5.5 Hz, 1H), 7.13 (d, J=1.5, 8.0 Hz, 1H), 6.84 (d, J=5.0, 7.5 Hz, 1H), 3.10-2.92 (m, 5H), 2.59 (d, J=4.5 Hz, 2H), 2.21-2.02 (m, 5H), 1.17-1.07 (m, 6H), 0.81 (t, J=7.5 Hz, 3H).

### Example 2.26. (R)-5-ethyl-8,8-dimethyl-5-(3-(5,6,7,8-tetrahydroquinolin-4-yl)phenyl)-5,8,9,10-tetrahydrobenzo[b][1,8]naphthyridin-6(7H)-one. (3A)

### (S)-5-ethyl-8,8-dimethyl-5-(3-(5,6,7,8-tetrahydroquinolin-4-yl)phenyl)-5,8,9,10-tetrahydrobenzo[b][1,8]naphthyridin-6(7H)-one. (3B)

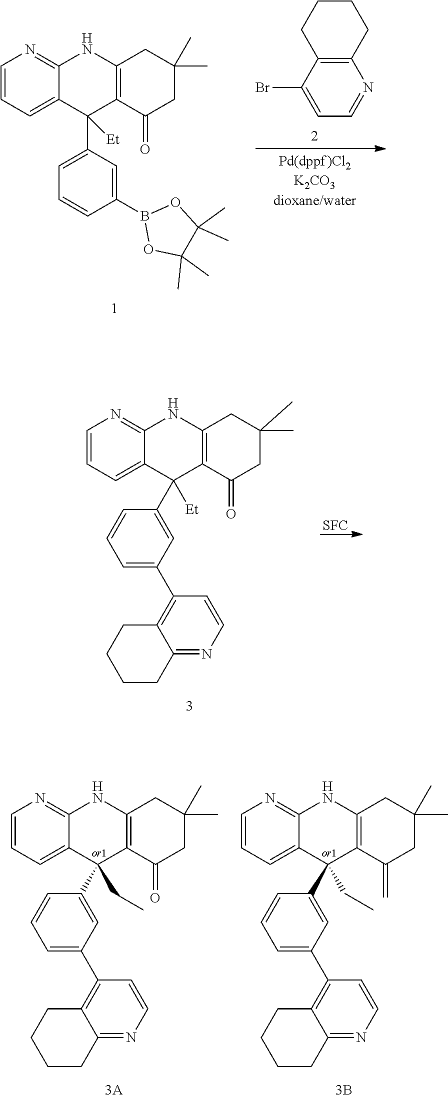

Compound 1 (40.00 mg, 87.26 μmol), compound 2 (24.06 mg, 113.44 μmol), Pd(dppf)Cl₂ (12.77 mg, 17.45 μmol) and K₂CO₃ (36.18 mg, 261.78 μmol) were added into Dioxane (4.00 mL)/H₂O (499.94 μL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to (20.00 mg, 32.35 μmol, 51.47% yield) as white solid.

LCMS: (ES⁺) M/Z (M+H)⁺: 464.1.

Racemic 3 (20.00 mg, 32.35 μmol) was separated by SFC (DAICEL CHIRALPAK AD (250 mm×30 mm, 10 μm); 0.1% NH₃H₂O ETOH; 50%; Flow Rate: 70 ml/min) to give:

(R)-5-ethyl-8,8-dimethyl-5-(3-(5,6,7,8-tetrahydroquinolin-4-yl)phenyl)-5,8,9,10-tetrahydrobenzo[b][1,8]naphthyridin-6(7H)-one. (3A) (8.00 mg, 17.26 μmol, 40.00% yield) as a white solid.

LCMS: tR=1.666 min., (ES⁺) M/Z (M+H)⁺: 464.2.

HPLC: (purity: 100%).

¹H NMR: (500 MHz, methanol-d4) δ=8.24 (d, J=5.0 Hz, 1H), 7.97 (d, J=1.5, 5.0 Hz, 1H), 7.47 (d, J=8.0 Hz, 1H), 7.34 (t, J=7.5 Hz, 1H), 7.30 (s, 1H), 7.13 (d, J=1.5, 8.0 Hz, 1H), 7.05-7.01 (m, 2H), 6.84 (d, J=5.0, 7.5 Hz, 1H), 3.01 (d, J=7.5, 13.0 Hz, 1H), 2.93 (t, J=6.5 Hz, 2H), 2.57 (s, 2H), 2.55-2.49 (m, 2H), 2.21-2.05 (m, 3H), 1.94-1.83 (m, 2H), 1.73-1.64 (m, 2H), 1.17-1.07 (m, 6H), 0.80 (t, J=7.5 Hz, 3H).

(S)-5-ethyl-8,8-dimethyl-5-(3-(5,6,7,8-tetrahydroquinolin-4-yl)phenyl)-5,8,9,10-tetrahydrobenzo[b][1,8]naphthyridin-6(7H)-one. (3B) (9.00 mg, 19.41 μmol, 45.00% yield) as a white solid.

LCMS: tR=2.426 min., (ES⁺) M/Z (M+H)⁺: 464.2.

HPLC: (purity: 100%).

¹H NMR: (500 MHz, methanol-d4) δ=8.25 (d, J=5.0 Hz, 1H), 7.97 (d, J=1.5, 5.0 Hz, 1H), 7.47 (d, J=8.0 Hz, 1H), 7.34 (t, J=7.5 Hz, 1H), 7.30 (s, 1H), 7.13 (d, J=1.5, 8.0 Hz, 1H), 7.06-7.01 (m, 2H), 6.84 (d, J=5.0, 7.5 Hz, 1H), 3.01 (d, J=7.5, 13.0 Hz, 1H), 2.93 (t, J=6.5 Hz, 2H), 2.57 (s, 2H), 2.55-2.48 (m, 2H), 2.20-2.04 (m, 3H), 1.93-1.84 (m, 2H), 1.68 (t, J=6.5 Hz, 2H), 1.17-1.07 (m, 6H), 0.80 (t, J=7.5 Hz, 3H).

### Example 2.27. (R)-5-ethyl-5-(3-(1-isopropyl-1H-imidazol-5-yl)phenyl)-8,8-dimethyl-5,8,9,10-tetrahydrobenzo[b][1,8]naphthyridin-6(7H)-one. (3A)

### (S)-5-ethyl-5-(3-(1-isopropyl-1H-imidazol-5-yl)phenyl)-8,8-dimethyl-5,8,9,10-tetrahydrobenzo[b][1,8]naphthyridin-6(7H)-one. (3B)

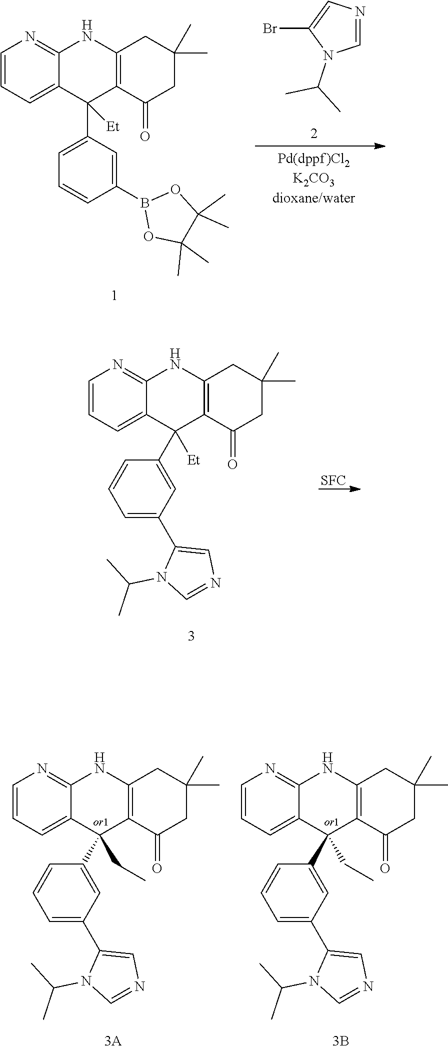

Compound 1 (70.00 mg, 152.71 μmol), compound 2 (37.53 mg, 198.52 μmol), Pd(dppf)Cl₂ (22.35 mg, 30.54 μmol) and K₂CO₃ (63.32 mg, 458.13 μmol) were added into Dioxane (4.00 mL)/H₂O (399.96 μL). The mixture was stirred at 90° C. for 16 hours under N₂ atmosphere. LCMS showed starting material was consumed completely and two main peaks with desired MS was detected. The mixture was concentrated, and the residue was diluted with water (10 mL) and extracted with EtOAc (10 mL×3). The combined organic layer was washed with brine (20 mL), dried over Na₂SO₄, filtered, and concentrated. The residue was purified by prep-HPLC (Column: Xtimate C18 150×25 mm×5 μm; Condition: water (10 mM NH₄HCO₃)-ACN; 23%-53%) to 3 (15.00 mg, 22.29% yield) as white solid.

LCMS: (ES⁺) M/Z (M+H)⁺: 441.1.

Racemic 3 (20.00 mg, 45.39 μmol) was separated by SFC (DAICEL CHIRALPAK AD (250 mm×30 mm, 10 μm); 0.1% NH₃H₂O ETOH; 50%; Flow Rate: 70 ml/min) to give:

(R)-5-ethyl-5-(3-(1-isopropyl-1H-imidazol-5-yl)phenyl)-8,8-dimethyl-5,8,9,10-tetrahydrobenzo[b][1,8]naphthyridin-6(7H)-one. (3A) (5.00 mg, 25.00% yield) as a white solid.

LCMS: tR=1.423 min., (ES⁺) M/Z (M+H)⁺: 441.2.

HPLC: (purity: 100%).

¹H NMR: (500 MHz, methanol-d4) δ=7.98 (d, J=1.5, 5.0 Hz, 1H), 7.86 (s, 1H), 7.48 (d, J=8.0 Hz, 1H), 7.38-7.32 (m, 2H), 7.12 (d, J=1.5, 7.5 Hz, 2H), 6.90-6.82 (m, 2H), 4.33-4.24 (m, 1H), 3.04 (d, J=7.5, 13.0 Hz, 1H), 2.58 (s, 2H), 2.21-2.04 (m, 3H), 1.37 (d, J=6.5, 12.5 Hz, 6H), 1.16-1.06 (m, 6H), 0.81 (t, J=7.5 Hz, 3H).

(S)-5-ethyl-5-(3-(1-isopropyl-1H-imidazol-5-yl)phenyl)-8,8-dimethyl-5,8,9,10-tetrahydrobenzo[b][1,8]naphthyridin-6(7H)-one. (3B) (5.00 mg, 25.00% yield) as a white solid.

LCMS: tR=2.191 min., (ES⁺) M/Z (M+H)⁺: 441.2.

HPLC: (purity: 100%).

¹H NMR: (500 MHz, methanol-d4) δ=7.98 (d, J=1.5, 5.0 Hz, 1H), 7.85 (s, 1H), 7.48 (d, J=8.0 Hz, 1H), 7.38-7.32 (m, 2H), 7.12 (d, J=1.5, 7.5 Hz, 2H), 6.91-6.81 (m, 2H), 4.32-4.25 (m, 1H), 3.04 (d, J=7.5, 13.0 Hz, 1H), 2.58 (s, 2H), 2.20-2.03 (m, 3H), 1.37 (d, J=6.5, 12.7 Hz, 6H), 1.17-1.06 (m, 6H), 0.81 (t, J=7.5 Hz, 3H).

### Example 2.28. (R)-5-ethyl-3-fluoro-8,8-dimethyl-5-(3-(3-methylpyridin-4-yl)phenyl)-5,8,9,10-tetrahydrobenzo[b][1,8]naphthyridin-6(7H)-one. (3A)

### (S)-5-ethyl-3-fluoro-8,8-dimethyl-5-(3-(3-methylpyridin-4-yl)phenyl)-5,8,9,10-tetrahydrobenzo[b][1,8]naphthyridin-6(7H)-one. (3B)

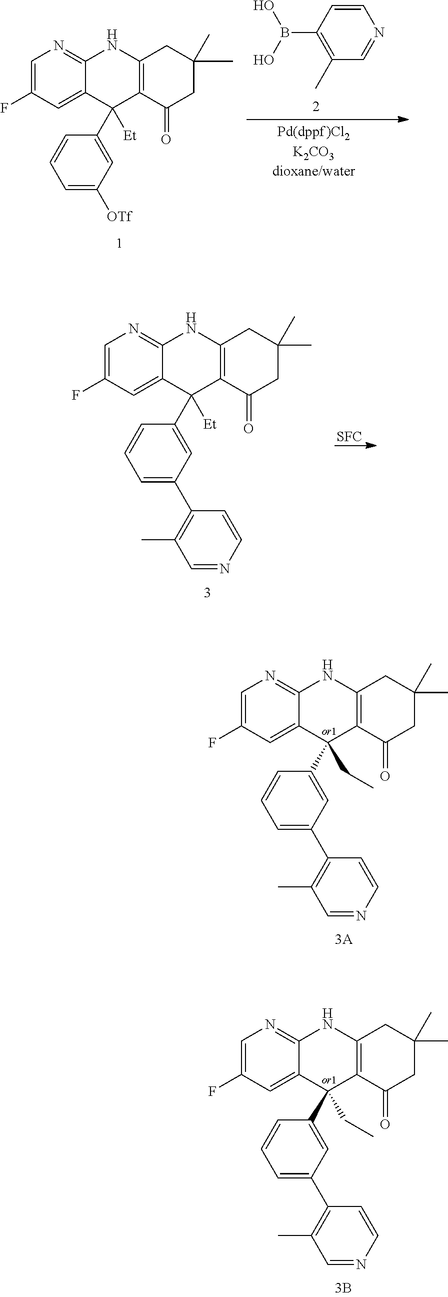

Compound 1 (100.00 mg, 200.61 μmol), compound 2 (41.21 mg, 300.91 μmol) and K₂CO₃ (69.31 mg, 501.53 μmol) were added into Dioxane (4.00 mL) and H₂O (1.00 mL). The reaction suspension was bubbled with N₂ for 1 min, then Pd(dppf)Cl₂·DCM (16.38 mg, 20.06 μmol) was added. The reaction mixture was stirred at 90° C. for 5 h under N₂ atmosphere. TLC (EtOAc, 254 mu) showed the reactant 1 was consumed and a new spot was observed. The mixture was concentrated, and the residue was purified by flash silica gel chromatography (EtOAc) to give compound 2 (80.00 mg, 90.32% yield) as a yellow solid.

¹H NMR: (400 MHz, MeOD) δ=8.46-8.34 (m, 2H), 7.90 (d, J=2.8 Hz, 1H), 7.43-7.36 (m, 2H), 7.27 (d, J=5.2 Hz, 2H), 7.13 (d, J=7.2 Hz, 1H), 6.93 (m, 1H), 3.03 (m, 1H), 2.57 (d, J=2.4 Hz, 2H), 2.20-2.05 (m, 3H), 1.15-1.07 (m, 6H), 0.82 (t, J=7.2 Hz, 3H).

Racemic 3 (80.00 mg, 181.18 μmol) was separated by SFC (DAICEL CHIRALCEL OJ-H (250 mm×30 mm, 5 μm); 0.1% NH₃H₂O ETOH; 20%; Flow Rate: 50 ml/min) and prep-HPLC (Column Welch Xtimate C18 150×30 mm×5 μm, Condition water (0.225% FA)-ACN) to give:

(R)-5-ethyl-3-fluoro-8,8-dimethyl-5-(3-(3-methylpyridin-4-yl)phenyl)-5,8,9,10-tetrahydrobenzo[b][1,8]naphthyridin-6(7H)-one. (3A) (12.00 mg, 15.00% yield) as a white solid.

LCMS: tR=1.118 min., (ES⁺) M/Z (M+H)⁺: 442.1.

HPLC: (purity: 100%).

¹H NMR: (500 MHz, methanol-d4) δ=8.42 (s, 1H), 8.36 (d, J=5.0 Hz, 1H), 7.90 (d, J=2.5 Hz, 1H), 7.49 (d, J=8.5 Hz, 1H), 7.42-7.37 (m, 2H), 7.26 (d, J=5.0 Hz, 1H), 7.14 (d, J=7.5 Hz, 1H), 6.94 (m, 1H), 3.08-2.98 (m, 1H), 2.61-2.53 (m, 2H), 2.22 (s, 3H), 2.19-2.07 (m, 3H), 1.14 (s, 3H), 1.09 (s, 3H), 0.82 (t, J=7.5 Hz, 3H).

(S)-5-ethyl-3-fluoro-8,8-dimethyl-5-(3-(3-methylpyridin-4-yl)phenyl)-5,8,9,10-tetrahydrobenzo[b][1,8]naphthyridin-6(7H)-one. (3B) (18.00 mg, 21.74% yield) as a white solid.

LCMS: tR=1.220 min., (ES⁺) M/Z (M+H)⁺: 442.1.

HPLC: (purity: 96.60%).

¹H NMR: (500 MHz, methanol-d4) δ=8.41 (s, 1H), 8.36 (d, J=5.0 Hz, 1H), 7.90 (d, J=2.5 Hz, 1H), 7.49 (d, J=8.0 Hz, 1H), 7.42-7.37 (m, 2H), 7.26 (d, J=5.0 Hz, 1H), 7.13 (d, J=7.5 Hz, 1H), 6.93 (m, 1H), 3.07-2.99 (m, 1H), 2.61-2.53 (m, 2H), 2.22 (s, 3H), 2.19-2.07 (m, 3H), 1.14 (s, 3H), 1.09 (s, 3H), 0.82 (t, J=7.5 Hz, 3H).

### Example 2.29. (R)-5-ethyl-3-fluoro-5-(3-(3-methoxypyridin-4-yl)phenyl)-8,8-dimethyl-5,8,9,10-tetrahydrobenzo[b][1,8]naphthyridin-6(7H)-one. (3A)

### (S)-5-ethyl-3-fluoro-5-(3-(3-methoxypyridin-4-yl)phenyl)-8,8-dimethyl-5,8,9,10-tetrahydrobenzo[b][1,8]naphthyridin-6(7H)-one. (3B)

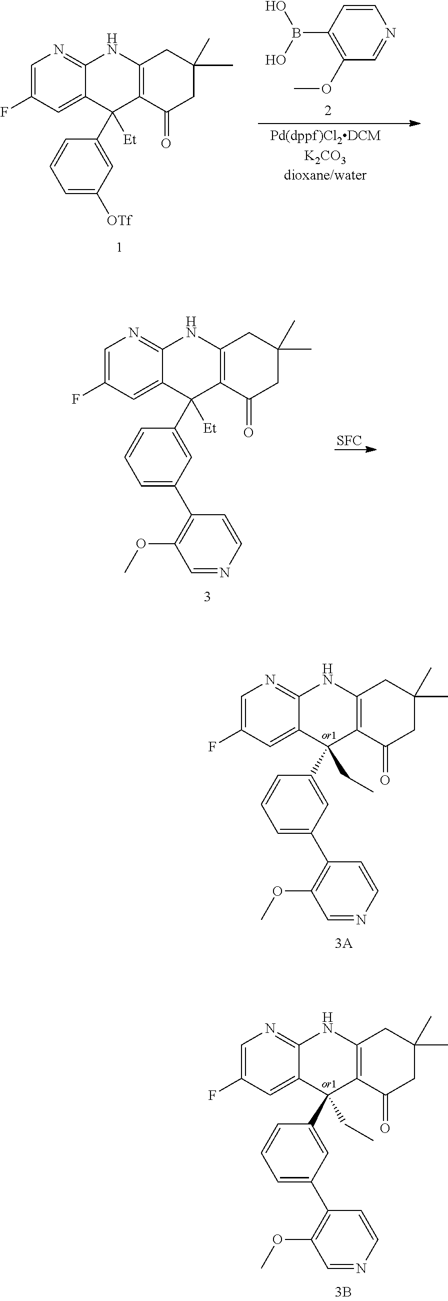 [SMILES: CCC1(c2cccc(OS(=O)(=O)C(F)(F)F)c2)C2=C(CC(C)(C)CC2=O)Nc2ncc(F)cc21 ; COc1cnccc1B(O)O ; CCC1(c2cccc(-c3ccncc3OC)c2)C2=C(CC(C)(C)CC2=O)Nc2ncc(F)cc21 ; "SFC" ; CC[C@]1(c2cccc(-c3ccncc3OC)c2)C2=C(CC(C)(C)CC2=O)Nc2ncc(F)cc21 ; CC[C@@]1(c2cccc(-c3ccncc3OC)c2)C2=C(CC(C)(C)CC2=O)Nc2ncc(F)cc21]

Compound 1 (100.00 mg, 200.61 μmol), compound 2 (46.02 mg, 300.91 μmol) and K₂CO₃ (69.31 mg, 501.53 μmol) were added into Dioxane (4.00 mL) and H₂O (1.00 mL). The reaction suspension was bubbled with N₂ for 1 min, then Pd(dppf)Cl₂·DCM (16.38 mg, 20.06 μmol) was added. The reaction mixture was stirred at 90° C. for 5 h under N₂ atmosphere. TLC (EtOAc 254 nm) showed the reactant 1 was consumed and a new spot was observed. The mixture was concentrated, and the residue was purified by flash silica gel chromatography (EtOAc) to give compound 3 (80.00 mg, 87.16% yield) as a yellow solid.

¹H NMR: (400 MHz, MeOD) δ=8.32 (s, 1H), 8.21 (d, J=4.8 Hz, 1H), 7.90 (d, J=2.8 Hz, 1H), 7.62 (s, 1H), 7.50-7.44 (m, 1H), 7.36-7.33 (m, 3H), 6.94 (m, 1H), 3.86 (s, 3H), 3.09-2.99 (m, 1H), 2.57 (s, 2H), 2.22-2.07 (m, 3H), 1.12 (d, J=16.0 Hz, 6H), 0.82 (t, J=7.2 Hz, 3H).

LCMS: ((ES⁺) M/Z (M+H)⁺: 458.1).

Racemic 3 (80.00 mg, 174.85 μmol) was separated by SFC (DAICEL CHIRALPAK AD-H (250 mm×30 mm, 5 μm); 0.1% NH₃H₂O ETOH; 30%; Flow Rate: 50 ml/min) to give:

(R)-5-ethyl-3-fluoro-5-(3-(3-methoxypyridin-4-yl)phenyl)-8,8-dimethyl-5,8,9,10-tetrahydrobenzo[b][1,8]naphthyridin-6(7H)-one. (3A) (16.00 mg, 18.92% yield, 94.58% purity, Rt=1.370 min) as a white solid.

LCMS: tR=1.370 min., (ES+) M/Z (M+H)+: 458.1.

HPLC: (purity: 94.58%).

¹H NMR: (500 MHz, MeOD) δ=8.32 (s, 1H), 8.20 (d, J=5.0 Hz, 1H), 7.89 (d, J=2.5 Hz, 1H), 7.61 (s, 1H), 7.50-7.43 (m, 1H), 7.36-7.32 (m, 3H), 6.94 (m, 1H), 3.86 (s, 3H), 3.08-3.01 (m, 1H), 2.57 (s, 2H), 2.20-2.07 (m, 3H), 1.14 (s, 3H), 1.10 (s, 3H), 0.82 (t, J=7.5 Hz, 3H).

(S)-5-ethyl-3-fluoro-5-(3-(3-methoxypyridin-4-yl)phenyl)-8,8-dimethyl-5,8,9,10-tetrahydrobenzo[b][1,8]naphthyridin-6(7H)-one. (3B) (20.00 mg, 24.82% yield) as a white solid.

LCMS: tR=1.666 min., (ES+) M/Z (M+H)+: 458.1.

HPLC: (purity: 99.30%).

¹H NMR: (500 MHz, methanol-d4) δ=8.32 (s, 1H), 8.20 (d, J=5.0 Hz, 1H), 7.90 (d, J=2.5 Hz, 1H), 7.61 (s, 1H), 7.46 (m, 1H), 7.37-7.32 (m, 3H), 6.94 (m, 1H), 3.86 (s, 3H), 3.09-3.00 (m, 1H), 2.62-2.53 (m, 2H), 2.19-2.07 (m, 3H), 1.14 (s, 3H), 1.10 (s, 3H), 0.82 (t, J=7.5 Hz, 3H).

### Example 2.30. (R)-5-(3-(1,3-dimethyl-1H-pyrazol-4-yl)phenyl)-5-ethyl-3-fluoro-8,8-dimethyl-5,8,9,10-tetrahydrobenzo[b][1,8]naphthyridin-6(7H)-one. (2A)

### (S)-5-(3-(1,3-dimethyl-1H-pyrazol-4-yl)phenyl)-5-ethyl-3-fluoro-8,8-dimethyl-5,8,9,10-tetrahydrobenzo[b][1,8]naphthyridin-6(7H)-one. (2B)

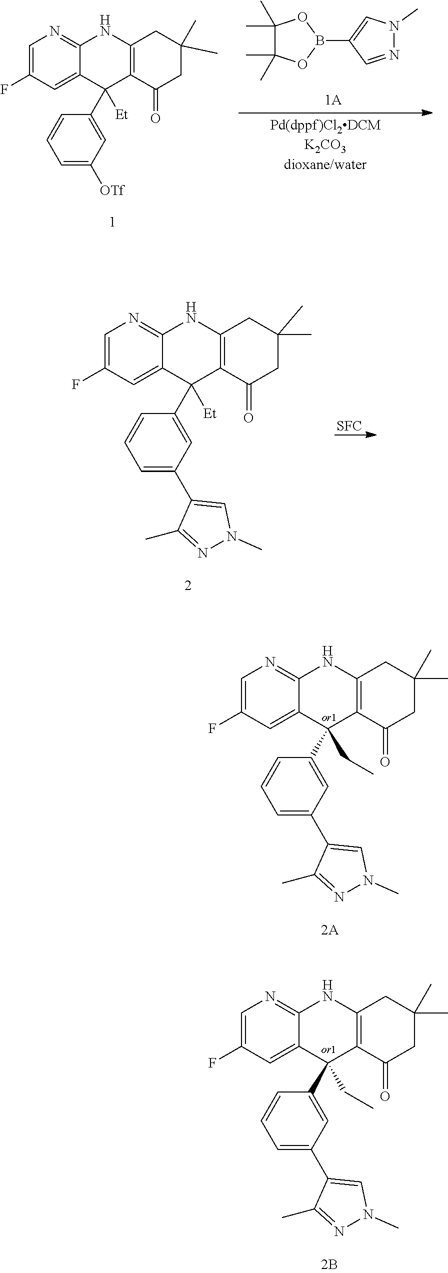

Compound 1 (100.00 mg, 200.61 μmol), compound 1A (66.83 mg, 300.91 μmol) and K₂CO₃ (69.31 mg, 501.53 μmol) were added into Dioxane (4.00 mL) and H₂O (1.00 mL). The reaction suspension was bubbled with N₂ for 1 min, then Pd(dppf)Cl₂·DCM (16.38 mg, 20.06 □mol) was added. The reaction mixture was stirred at 90° C. for 5 h under N₂ atmosphere. TLC (EtOAc, nm) showed the reactant 1 was consumed and a new spot was observed. The mixture was concentrated, and the residue was purified by flash silica gel chromatography (EtOAc) to give compound 2 (79.00 mg, crude) as a yellow solid.

LCMS: (ES⁺) M/Z (M+H)⁺: 445.1.

Racemic 2 (79.00 mg, 177.71 μmol) was separated by SFC (DAICEL CHIRALPAK AD (250 mm×30 mm, 10 μm); 0.1% NH₃H₂O IPA; 40%; Flow Rate: 80 ml/min) to give:

(R)-5-(3-(1,3-dimethyl-1H-pyrazol-4-yl)phenyl)-5-ethyl-3-fluoro-8,8-dimethyl-5,8,9,10-tetrahydrobenzo[b][1,8]naphthyridin-6(7H)-one. (2A) (21.00 mg, 26.18% yield) as a white solid.

LCMS: tR=1.437 min., (ES⁺) M/Z (M+H)⁺: 445.1.

HPLC: (purity: 98.48%).

¹H NMR: (500 MHz, MeOD) δ=7.88 (d, J=2.5 Hz, 1H), 7.63 (s, 1H), 7.45-7.38 (m, 1H), 7.31-7.24 (m, 2H), 7.15 (m, 1H), 6.92 (m, 1H), 3.83 (s, 3H), 3.10-3.01 (m, 1H), 2.57 (d, J=2.0 Hz, 2H), 2.26 (s, 3H), 2.18-2.06 (m, 3H), 1.14 (s, 3H), 1.10 (s, 3H), 0.82 (t, J=7.5 Hz, 3H).

(S)-5-(3-(1,3-dimethyl-1H-pyrazol-4-yl)phenyl)-5-ethyl-3-fluoro-8,8-dimethyl-5,8,9,10-tetrahydrobenzo[b][1,8]naphthyridin-6(7H)-one. (2B) (24.00 mg, 29.84% yield) as a brown solid.

LCMS: tR=1.817 min., (ES⁺) M/Z (M+H)⁺: 445.1.

HPLC: (purity: 98.23%).

¹H NMR: (500 MHz, MeOD) δ=7.88 (d, J=2.5 Hz, 1H), 7.63 (s, 1H), 7.42 (s, 1H), 7.30-7.25 (m, 2H), 7.17-7.13 (m, 1H), 6.92 (m, 1H), 3.83 (s, 3H), 3.10-3.01 (m, 1H), 2.57 (d, J=2.0 Hz, 2H), 2.26 (s, 3H), 2.18-2.06 (m, 3H), 1.14 (s, 3H), 1.10 (s, 3H), 0.82 (t, J=7.5 Hz, 3H).

### Example 2.31. (R)-5-ethyl-3-fluoro-5-(3-(1-isopropyl-1H-imidazol-5-yl)phenyl)-8,8-dimethyl-5,8,9,10-tetrahydrobenzo[b][1,8]naphthyridin-6(7H)-one. (3A)

### (S)-5-ethyl-3-fluoro-5-(3-(1-isopropyl-1H-imidazol-5-yl)phenyl)-8,8-dimethyl-5,8,9,10-tetrahydrobenzo[b][1,8]naphthyridin-6(7H)-one. (3B)

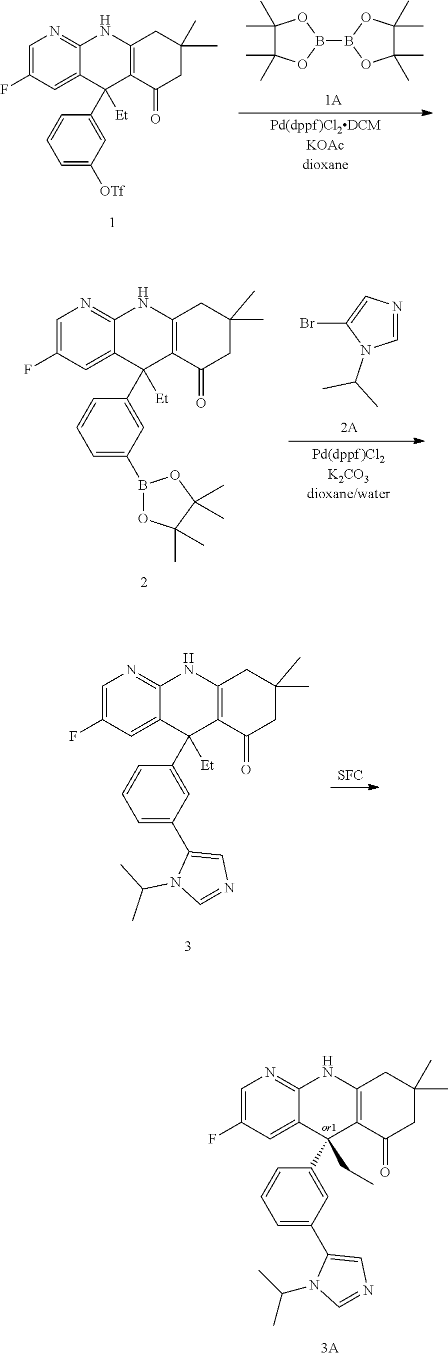

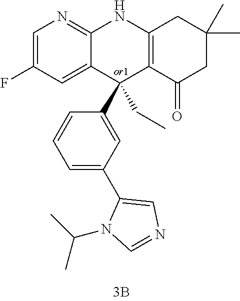 [SMILES: CC[C@@]1(c2cccc(-c3cncn3C(C)C)c2)C2=C(CC(C)(C)CC2=O)Nc2ncc(F)cc21]

### Step 1: 5-ethyl-3-fluoro-8,8-dimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one. (2)

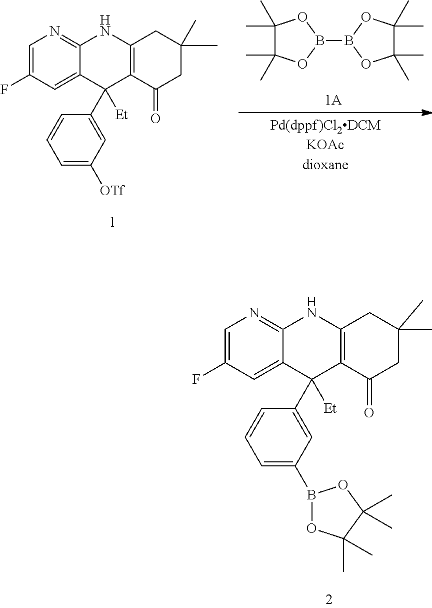

Compound 1 (30.00 mg, 60.18 μmol), compound 1A (280.18 mg, 1.10 mmol) and KOAc (129.94 mg, 1.32 mmol) were added into Dioxane (8.00 mL). The reaction suspension was bubbled with N₂ for 1 min, then Pd(dppf)Cl₂·DCM (36.04 mg, 44.13 μmol) was added. The reaction mixture was stirred at 90° C. for 16 h under N₂ atmosphere. LCMS showed the reactant 1 was consumed and desired compound was detected. The mixture was concentrated, and the residue was purified by flash silica gel chromatography (EtOAc) to give compound 2 (120.00 mg, 57.08% yield) as a yellow solid.

¹H NMR: (500 MHz, MeOD) δ 7.89-7.84 (m, 1H), 7.79 (s, 1H), 7.56-7.52 (m, 1H), 7.49 (d, J=7.5 Hz, 1H), 7.30-7.25 (m, 1H), 6.85 (m, 1H), 3.04-2.94 (m, 1H), 2.62-2.53 (m, 2H), 2.18-2.00 (m, 3H), 1.20 (s, 12H), 1.13 (d, J=7.5 Hz, 6H).

### Step 2: (R)-5-ethyl-3-fluoro-5-(3-(1-isopropyl-1H-imidazol-5-yl)phenyl)-8,8-dimethyl-5,8,9,10-tetrahydrobenzo[b][1,8]naphthyridin-6(7H)-one. (3A)

### (S)-5-ethyl-3-fluoro-5-(3-(1-isopropyl-1H-imidazol-5-yl)phenyl)-8,8-dimethyl-5,8,9,10-tetrahydrobenzo[b][1,8]naphthyridin-6(7H)-one. (3B)

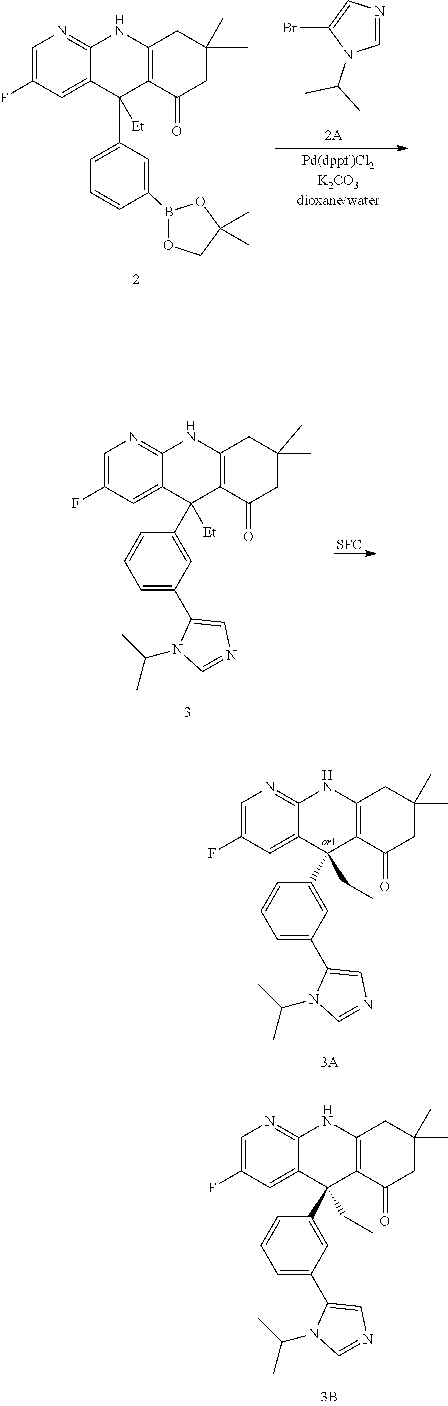

Compound 2 (60.00 mg, 125.95 μmol), compound 2A (36.00 mg, 190.43 μmol) and K₂CO₃ (43.52 mg, 314.87 μmol) were added into Dioxane (2.00 mL) and H₂O (500.00 μL). The reaction suspension was bubbled with N₂ for 1 min, then Pd(dppf)Cl₂ DCM (10.29 mg, 12.59 μmol) was added. The reaction mixture was stirred at 90° C. for 5 h under N₂ atmosphere. TLC (EtOAc/MeOH=19/1 254 nm) showed the reactant 2 was consumed and a new spot was observed. The mixture was concentrated, and the residue was purified by flash silica gel chromatography (EtOAc/MeOH=19/1) to give compound 3 (28.00 mg, 48.48% yield) as a yellow solid.

LCMS: ((ES+) M/Z (M+H)+: 459.1).

Compound 3 (28.00 mg, 61.06 μmol) was separated by SFC (DAICEL CHIRALPAK AD-H (250 mm×30 mm, 5 μm); 0.1% NH₃H₂O ETOH; 35%; Flow Rate: 50 ml/min) to give:

(R)-5-ethyl-3-fluoro-5-(3-(1-isopropyl-1H-imidazol-5-yl)phenyl)-8,8-dimethyl-5,8,9,10-tetrahydrobenzo[b][1,8]naphthyridin-6(7H)-one. (3A) (9.00 mg, 32.14% yield) as a white solid.

LCMS: tR=3.878 min., (ES⁺) M/Z (M+H)⁺: 459.1.

HPLC: (purity: 100%).

¹H NMR: (500 MHz, MeOD) δ=7.90 (d, J=2.5 Hz, 1H), 7.85 (s, 1H), 7.48 (d, J=8.0 Hz, 1H), 7.40-7.34 (m, 2H), 7.14 (d, J=7.5 Hz, 1H), 6.94-6.88 (m, 2H), 4.31 (m, 1H), 3.10-3.01 (m, 1H), 2.57 (s, 2H), 2.20-2.04 (m, 3H), 1.38 (m, 6H), 1.14 (s, 3H), 1.08 (s, 3H), 0.82 (t, J=7.5 Hz, 3H).

(S)-5-ethyl-3-fluoro-5-(3-(1-isopropyl-1H-imidazol-5-yl)phenyl)-8,8-dimethyl-5,8,9,10-tetrahydrobenzo[b][1,8]naphthyridin-6(7H)-one. (3B) (8.00 mg, 28.57% yield) as a white solid.

LCMS: tR=4.624 min., (ES⁺) M/Z (M+H)⁺: 459.1.

HPLC: (purity: 100%).

¹H NMR: (400 MHz, MeOD) δ=8.13 (s, 1H), 7.91 (d, J=2.8 Hz, 1H), 7.50 (d, J=8.0 Hz, 1H), 7.42-7.36 (m, 2H), 7.17 (d, J=7.6 Hz, 1H), 7.04 (s, 1H), 6.92 (m, 1H), 4.41-4.32 (m, 1H), 3.09-2.97 (m, 1H), 2.57 (s, 2H), 2.20-2.04 (m, 3H), 1.41 (m, 6H), 1.14 (s, 3H), 1.09 (s, 3H), 0.82 (t, J=7.2 Hz, 3H).

### Example 2.32. (R)-5-ethyl-3-fluoro-5-(3-(3-fluoro-5-methoxypyridin-4-yl)phenyl)-8,8-dimethyl-5,8,9,10-tetrahydrobenzo[b][1,8]naphthyridin-6(7H)-one. (3A)

### (S)-5-ethyl-3-fluoro-5-(3-(3-fluoro-5-methoxypyridin-4-yl)phenyl)-8,8-dimethyl-5,8,9,10-tetrahydrobenzo[b][1,8]naphthyridin-6(7H)-one. (3B)

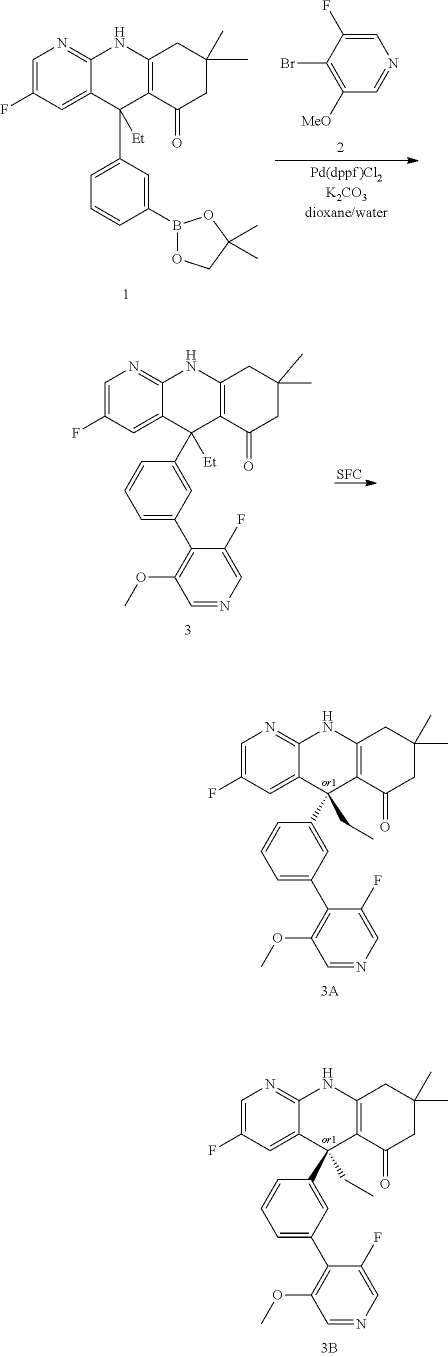

Compound 1 (150.00 mg, 314.87 μmol), compound 2 (104.00 mg, 504.83 μmol) and K₂CO₃ (108.79 mg, 787.18 μmol) were added into Dioxane (4.00 mL) and H₂O (1.00 mL). The reaction suspension was bubbled with N₂ for 1 min, then Pd(dppf)Cl₂·DCM (25.71 mg, 31.49 μmol) was added. The reaction mixture was stirred at 90° C. for 5 h under N₂ atmosphere. TLC (EtOAc, 254 mu) showed the reactant 1 was consumed and a new spot was observed. The mixture was concentrated, and the residue was purified by flash silica gel chromatography (EtOAc) and prep-HPLC (Column Welch Xtimate C18 150×30 mm×5 μm, Condition water (10 mM NH₄HCO₃)-ACN) to give compound 2 (70.00 mg, 46.75% yield) as a yellow solid.

LCMS: (ES⁺) M/Z (M+H)⁺: 476.1.

Racemic 3 (70.00 mg, 147.20 μmol) was separated by SFC (DAICEL CHIRALCEL OD-H (250 mm×30 mm, 5 μm); 0.1% NH₃H₂O ETOH; 25%; Flow Rate: 50 ml/min) to give:

(R)-5-ethyl-3-fluoro-5-(3-(3-fluoro-5-methoxypyridin-4-yl)phenyl)-8,8-dimethyl-5,8,9,10-tetrahydrobenzo[b][1,8]naphthyridin-6(7H)-one. (3A) (20.00 mg, 28.57% yield) as a white solid.

LCMS: tR=3.572 min., (ES⁺) M/Z (M+H)⁺: 476.1.

HPLC: (purity: 100%).

¹H NMR: (500 MHz, methanol-d4) δ=8.23 (s, 1H), 8.19 (s, 1H), 7.89 (d, J=2.5 Hz, 1H), 7.51 (d, J=8.0 Hz, 1H), 7.44 (d, J=1.0 Hz, 1H), 7.37 (t, J=8.0 Hz, 1H), 7.21 (d, J=6.5 Hz, 1H), 6.92 (dd, J=2.5, 9.5 Hz, 1H), 3.88-3.82 (m, 3H), 3.08-2.97 (m, 1H), 2.57 (d, J=2.5 Hz, 2H), 2.20-2.05 (m, 3H), 1.14 (s, 3H), 1.10 (s, 3H), 0.82 (t, J=7.5 Hz, 3H).

(S)-5-ethyl-3-fluoro-5-(3-(3-fluoro-5-methoxypyridin-4-yl)phenyl)-8,8-dimethyl-5,8,9,10-tetrahydrobenzo[b][1,8]naphthyridin-6(7H)-one. (3B) (22.00 mg, 31.43% yield) as a white solid.

LCMS: tR=3.759 min., (ES⁺) M/Z (M+H)⁺: 476.1.

HPLC: (purity: 100%).

¹H NMR: (500 MHz, methanol-d4) δ=8.23 (s, 1H), 8.19 (s, 1H), 7.89 (d, J=2.5 Hz, 1H), 7.51 (d, J=8.0 Hz, 1H), 7.44 (s, 1H), 7.37 (t, J=8.0 Hz, 1H), 7.21 (d, J=7.5 Hz, 1H), 6.92 (dd, J=2.5, 9.0 Hz, 1H), 3.85 (s, 3H), 3.06-2.98 (m, 1H), 2.57 (d, J=2.5 Hz, 2H), 2.19-2.06 (m, 3H), 1.14 (s, 3H), 1.10 (s, 3H), 0.82 (t, J=7.5 Hz, 3H).

### Example 2.33. (R)-5-(3-(2,3-dimethylpyridin-4-yl)phenyl)-5-ethyl-3-fluoro-8,8-dimethyl-5,8,9,10-tetrahydrobenzo[b][1,8]naphthyridin-6(7H)-one. (3A)

### (S)-5-(3-(2,3-dimethylpyridin-4-yl)phenyl)-5-ethyl-3-fluoro-8,8-dimethyl-5,8,9,10-tetrahydrobenzo[b][1,8]naphthyridin-6(7H)-one. (3B)

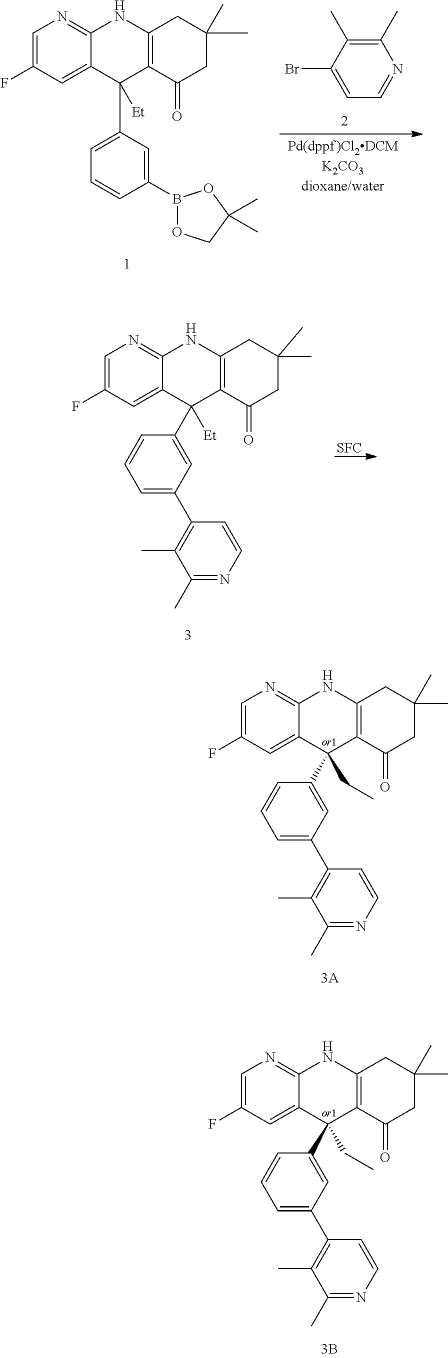

Compound 1 (60.00 mg, 125.95 μmol), compound 2 (35.15 mg, 188.93 μmol) and K₂CO₃ (43.52 mg, 314.88 μmol) were added into Dioxane (2.00 mL) and H₂O (500.00 μL). The reaction suspension was bubbled with N₂ for 1 min, then Pd(dppf)Cl₂ DCM (10.29 mg, 12.60 μmol) was added. The reaction mixture was stirred at 90° C. for 5 h under N₂ atmosphere. TLC (EtOAc, 254 mu) showed the reactant 1 was consumed and a new spot was observed. The mixture was concentrated, and the residue was purified by flash silica gel chromatography (EtOAc) and prep-TLC (EtOAc 254 nm) to give compound 2 (30.00 mg, 52.28% yield) as a yellow solid.

LCMS: (ES⁺) M/Z (M+H)⁺: 456.1.

Racemic 3 (30.00 mg, 65.85 μmol) was separated by SFC (DAICEL CHIRALPAK AD-H (250 mm×30 mm, 5 μm); 0.1% NH₃H₂O ETOH; 30%; Flow Rate: 50 ml/min) to give:

(R)-5-(3-(2,3-dimethylpyridin-4-yl)phenyl)-5-ethyl-3-fluoro-8,8-dimethyl-5,8,9,10-tetrahydrobenzo[b][1,8]naphthyridin-6(7H)-one. (3A) (12.00 mg, 39.08% yield) as a white solid.

LCMS: tR=1.250 min., (ES⁺) M/Z (M+H)⁺: 456.2.

HPLC: (purity: 97.70%).

¹H NMR: (500 MHz, MeOD) δ=8.21 (d, J=5.0 Hz, 1H), 7.89 (d, J=2.5 Hz, 1H), 7.48 (d, J=8.0 Hz, 1H), 7.38 (t, J=7.5 Hz, 1H), 7.32 (s, 1H), 7.08 (d, J=5.5 Hz, 2H), 6.93 (m, 1H), 3.06-2.98 (m, 1H), 2.61-2.53 (m, 5H), 2.19-2.07 (m, 6H), 1.14 (s, 3H), 1.09 (s, 3H), 0.82 (t, J=7.5 Hz, 3H).

(S)-5-(3-(2,3-dimethylpyridin-4-yl)phenyl)-5-ethyl-3-fluoro-8,8-dimethyl-5,8,9,10-tetrahydrobenzo[b][1,8]naphthyridin-6(7H)-one. (3B) (13.00 mg, 43.33% yield) as a white solid.

LCMS: tR=1.420 min., (ES⁺) M/Z (M+H)⁺: 456.1.

HPLC: (purity: 100%).

¹H NMR: (400 MHz, MeOD) δ=8.21 (d, J=5.0 Hz, 1H), 7.89 (d, J=2.5 Hz, 1H), 7.48 (d, J=8.5 Hz, 1H), 7.38 (t, J=7.5 Hz, 1H), 7.32 (s, 1H), 7.08 (d, J=5.5 Hz, 2H), 6.93 (m, 1H), 3.07-2.98 (m, 1H), 2.61-2.51 (m, 5H), 2.19-2.07 (m, 6H), 1.14 (s, 3H), 1.09 (s, 3H), 0.82 (t, J=7.5 Hz, 3H).

### Example 2.34. (R)-5-ethyl-3-fluoro-8,8-dimethyl-5-(3-(5,6,7,8-tetrahydroquinolin-4-yl)phenyl)-5,8,9,10-tetrahydrobenzo[b][1,8]naphthyridin-6(7H)-one. (3A)

### (S)-5-ethyl-3-fluoro-8,8-dimethyl-5-(3-(5,6,7,8-tetrahydroquinolin-4-yl)phenyl)-5,8,9,10-tetrahydrobenzo[b][1,8]naphthyridin-6(7H)-one. (3B)

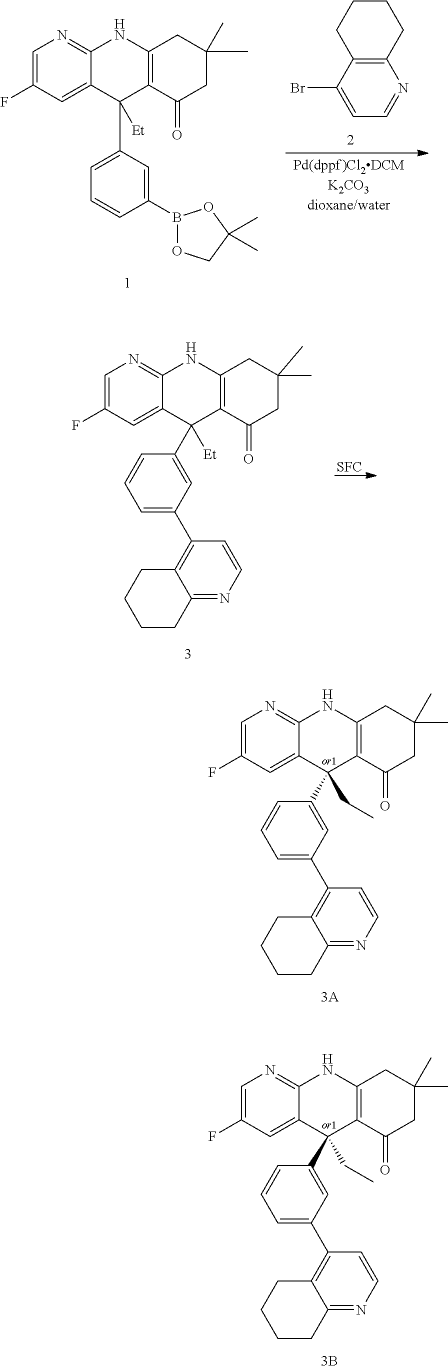 [SMILES: CCC1(c2cccc(B3OCC(C)(C)O3)c2)C2=C(CC(C)(C)CC2=O)Nc2ncc(F)cc21 ; Brc1ccnc2c1CCCC2 ; CCC1(c2cccc(-c3ccnc4c3CCCC4)c2)C2=C(CC(C)(C)CC2=O)Nc2ncc(F)cc21 ; "SFC" ; CC[C@]1(c2cccc(-c3ccnc4c3CCCC4)c2)C2=C(CC(C)(C)CC2=O)Nc2ncc(F)cc21 ; CC[C@@]1(c2cccc(-c3ccnc4c3CCCC4)c2)C2=C(CC(C)(C)CC2=O)Nc2ncc(F)cc21]

Compound 1 (150.0 mg, 314.9 μmol), compound 2 (104.0 mg, 490.4 μmol) and K₂CO₃ (108.8 mg, 787.2 μmol) were added into Dioxane (4 mL) and H₂O (1 mL). The reaction suspension was bubbled with N₂ for 1 min, and then Pd (dppf)Cl₂DCM (25.7 mg, 31.4 μmol) was added. The reaction mixture was stirred at 90° C. for 5 h under N₂ atmosphere. TLC (EtOAc 254 nm) showed the starting material was consumed and a new spot was observed. The mixture was concentrated, and the residue was purified by flash silica gel chromatography (EtOAc) and prep-HPLC (Column Welch Xtimate C18 150×30 mm×5 μm, Condition water (10 mM NH₄HCO₃)-ACN) to give compound 2 (70 mg, 46.16% yield) as a white solid.

LCMS: (ES⁺) M/Z (M+H)⁺: 482.2.

Racemic 3 (70 mg, 0.15 mmol) was separated by SFC (DAICEL CHIRALCEL OJ-H (250 mm×30 mm, 5 μm); 0.1% NH-3H₂O ETOH—CO₂; 25%, Flow Rate: 50 ml/min) to give:

(R)-5-ethyl-3-fluoro-8,8-dimethyl-5-(3-(5,6,7,8-tetrahydroquinolin-4-yl)phenyl)-5,8,9,10-tetrahydrobenzo[b][1,8]naphthyridin-6(7H)-one. (3A) (19 mg, 27.14% yield) as a white solid.

LCMS: tR=3.781 min., (ES⁺) M/Z (M+H)⁺: 482.6.

HPLC: (purity: 100%).

¹H NMR: (500 MHz, methanol-d4) δ=8.25 (d, J=5.0 Hz, 1H), 7.90 (d, J=2.5 Hz, 1H), 7.47 (d, J=8.0 Hz, 1H), 7.37 (t, J=7.5 Hz, 1H), 7.31 (s, 1H), 7.09-7.03 (m, 2H), 6.94-6.91 (m, 1H), 3.07-2.98 (m, 1H), 2.94 (t, J=6.5 Hz, 2H), 2.59-2.50 (m, 4H), 2.20-2.05 (m, 3H), 1.95-1.86 (m, 2H), 1.75-1.65 (m, 2H), 1.17-1.06 (m, 6H), 0.82 (t, J=7.5 Hz, 3H).

(S)-5-ethyl-3-fluoro-8,8-dimethyl-5-(3-(5,6,7,8-tetrahydroquinolin-4-yl)phenyl)-5,8,9,10-tetrahydrobenzo[b][1,8]naphthyridin-6(7H)-one. (3B) (27 mg, 38.57% yield) as a white solid.

LCMS: tR=4.058 min., (ES⁺) M/Z (M+H)⁺: 482.3.

HPLC: (purity: 100%).

¹H NMR: (500 MHz, methanol-d4) δ=8.26 (d, J=5.0 Hz, 1H), 7.90 (d, J=2.5 Hz, 1H), 7.47 (d, J=8.0 Hz, 1H), 7.37 (t, J=8.0 Hz, 1H), 7.31 (s, 1H), 7.09-7.02 (m, 2H), 6.94-6.91 (m, 1H), 3.06-2.98 (m, 1H), 2.94 (t, J=6.5 Hz, 2H), 2.58-2.50 (m, 4H), 2.21-2.14 (m, 1H), 2.12-2.05 (m, 2H), 1.94-1.86 (m, 2H), 1.74-1.66 (m, 2H), 1.16-1.06 (m, 6H), 0.82 (t, J=7.5 Hz, 3H).

### Example 3. Synthesis of Additional Select Compounds

The compound numbers recited in each one of Examples 3.1 to 3.22 apply only to each one of Examples 3.1 to 3.22, respectively.

### Example 3.1. (S)-3-(difluoromethyl)-5,8,8-trimethyl-6-oxo-5-phenyl-5,6,7,8,9,10-hexahydrobenzo[b][1,8]naphthyridine-4-carbonitrile (14A) and (R)-3-(difluoromethyl)-5,8,8-trimethyl-6-oxo-5-phenyl-5,6,7,8,9,10-hexahydrobenzo[b][1,8]naphthyridine-4-carbonitrile (14B)

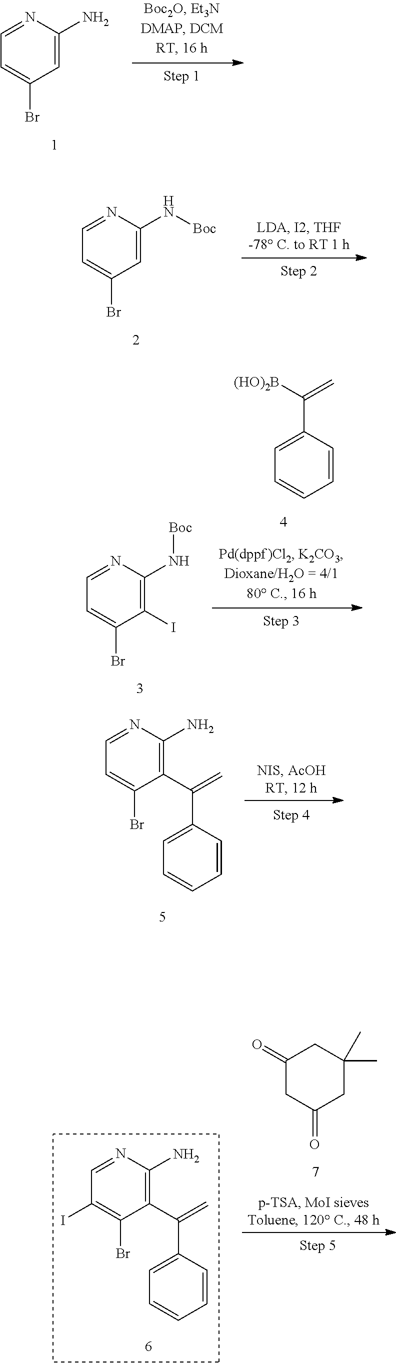

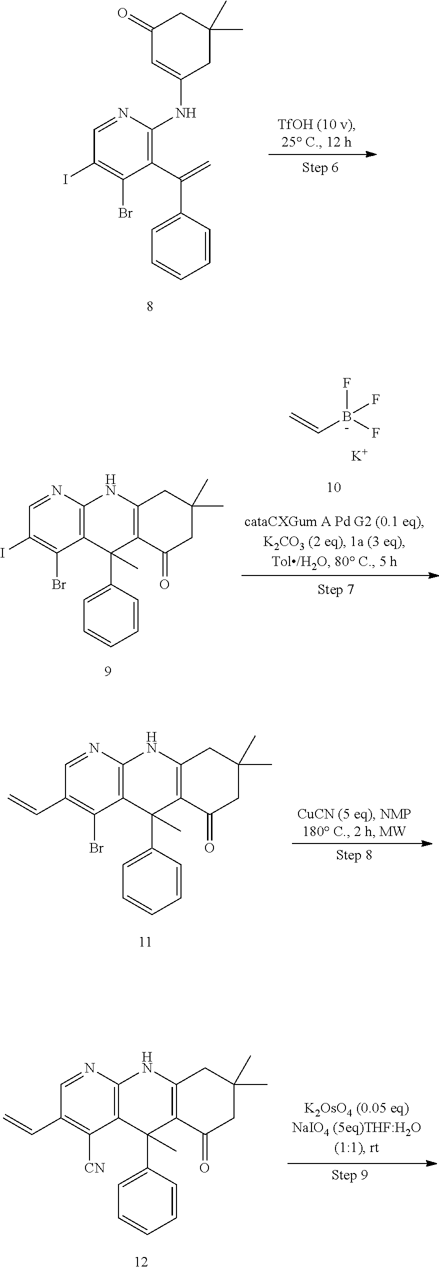

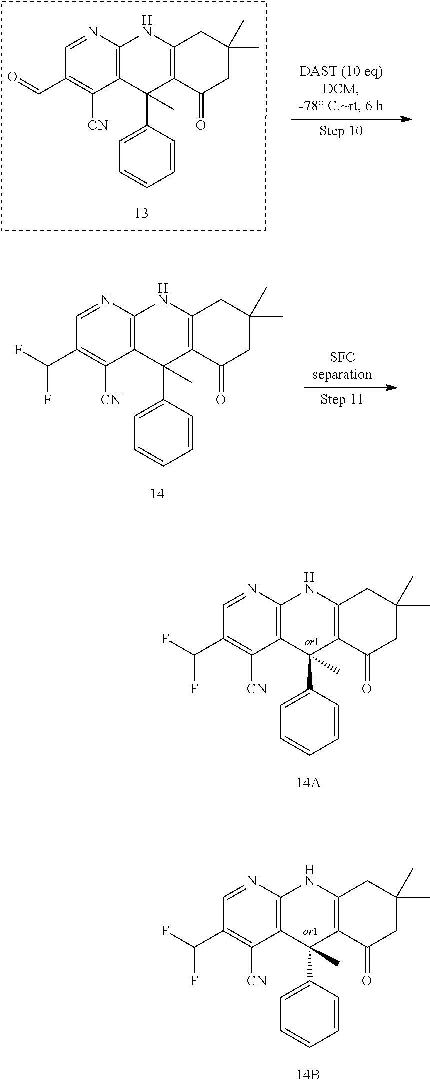 [SMILES: CC1(C)CC(=O)C2=C(C1)Nc1ncc(C=O)c(C#N)c1C2(C)c1ccccc1 ; CC1(C)CC(=O)C2=C(C1)Nc1ncc(C(F)F)c(C#N)c1C2(C)c1ccccc1 ; CC1(C)CC(=O)C2=C(C1)Nc1ncc(C(F)F)c(C#N)c1[C@]2(C)c1ccccc1 ; CC1(C)CC(=O)C2=C(C1)Nc1ncc(C(F)F)c(C#N)c1[C@@]2(C)c1ccccc1]

### Step 1: tert-butyl (4-bromopyridin-2-yl)carbamate (2)

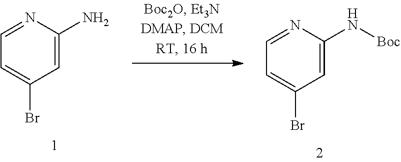 [SMILES: Nc1cc(Br)ccn1 ; CC(C)(C)OC(=O)Nc1cc(Br)ccn1]

To a stirred solution of 4-bromopyridin-2-amine (1) (10 g, 57.8 mmol, 1 eq) in DCM (150 mL) were sequentially added (Boc)₂O (13.3 mL, 57.8 mmol, 1.00 eq) and DMAP (1410 mg, 11.6 mmol, 0.2 eq) portion wise under ice cooled condition. Reaction mixture was allowed to stir at 25° C. for 16 h. On completion, reaction mixture was diluted with DCM, washed with aqueous NaHCO₃, water and brine. Organic layer was collected, dried over Na₂SO₄, filtered and concentrated to obtain a colorless crude, which was purified by CombiFlash column chromatography using 5-20% EtOAc in hexane gradient to afford tert-butyl N-(4-bromo-2-pyridyl)carbamate (2) (8.1 g, 29.4 mmol, 99% purity, 51% yield) as white solid.

LCMS: (ES⁺) m/z (M+H)⁺=272.7, Rt=3.63 min.

### Step 2: tert-butyl (4-bromo-3-iodopyridin-2-yl)carbamate (3)

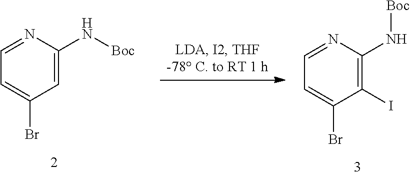 [SMILES: CC(C)(C)OC(=O)Nc1cc(Br)ccn1 ; CC(C)(C)OC(=O)Nc1nccc(Br)c1I]

To a stirred solution of tert-butyl N-(4-bromo-2-pyridyl)carbamate (2) (5 g, 18.3 mmol, 1.00 eq) in THF (90 mL) was added LDA (2 M in THF, 22.9 mL, 45.8 mmol, 2.50 eq) at −78° C. dropwise and the reaction was continued for 1 h. Iodine (5 g, 20.1 mmol, 1.10 eq) dissolved in THF (7 ml) was added dropwise to the RM under same temperature and the resulting mixture was allowed to stir for 30 min at 15° C. Reaction mixture was poured into saturated ammonium chloride solution, extracted with EtOAc (twice). Combined organic layer was washed sequentially with aqueous Na₂S₂O₃ solution, water and brine, dried over Na₂SO₄, filtered and concentrated under reduced pressure. Obtained residue was purified by CombiFlash column chromatography using 5-15% EtOAc-hexane gradient to afford tert-butyl N-(4-bromo-3-iodo-2-pyridyl)carbamate (3) (1.8 g, 4.42 mmol, 98% purity, 24% yield) as yellow solid.

LCMS: (ES⁺) m/z (M+H)⁺=399.0, Rt=1.90 min.

### Step 3: 4-bromo-3-(1-phenylvinyl)pyridin-2-amine (5)

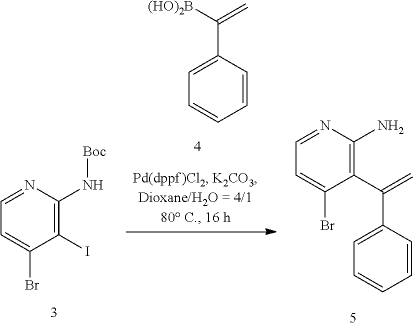 [SMILES: C=C(B(O)O)c1ccccc1 ; CC(C)(C)OC(=O)Nc1nccc(Br)c1I ; C=C(c1ccccc1)c1c(Br)ccnc1N]

To a degassed stirred solution of tert-butyl N-(4-bromo-3-iodo-2-pyridyl)carbamate (3) (7.5 g, 18.8 mmol, 1 eq) and 1-phenylvinylboronic acid (4) (3.33 g, 22.6 mmol, 1.20 eq) in 4:1 dioxane-water mixture (200 mL) were sequentially added K₂CO₃ (7.8 g, 56.4 mmol, 3 eq) and Pd(dppf)Cl₂ (1.4 g, 1.88 mmol, 0.1 eq) under inert atmosphere at RT. Resulting mixture was allowed to stir at 80° C. for 16 h. Reaction mixture was diluted with EtOAc, washed sequentially with water and brine, dried over Na₂SO₄, filtered and concentrated to obtain brownish crude. Crude was purified by CombiFlash column chromatography using 10-30% EtOAc-hexane gradient to afford 4-bromo-3-(1-phenylvinyl)pyridin-2-amine (5) (3.8 g, 10.5 mmol, 76% purity, 56% yield) as light yellow solid.

LCMS: (ES⁺) m/z (M+H)⁺=277.1, Rt=1.86 min.

### Step 4: 4-bromo-5-iodo-3-(1-phenylvinyl)pyridin-2-amine (6)

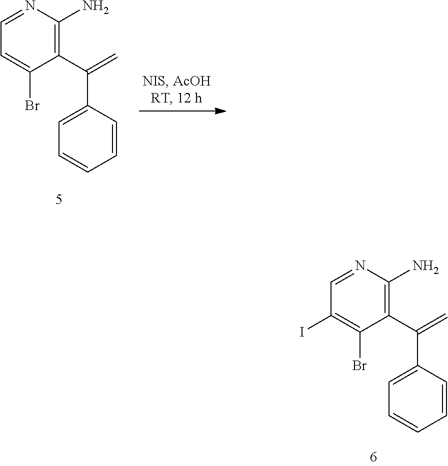 [SMILES: C=C(c1ccccc1)c1c(Br)ccnc1N ; C=C(c1ccccc1)c1c(N)ncc(I)c1Br]

To a stirred solution of 4-bromo-3-(1-phenylvinyl)pyridin-2-amine (5) (1 g, 3.63 mmol, 1 eq) in AcOH (10 mL) was portion wise added NIS (1.64 g, 7.27 mmol, 2 eq) at 0° C. under inert atmosphere. The resulting mixture was allowed to stir at 25° C. for 12 h. On completion, the reaction mixture was basified with aqueous sodium bicarbonate and extracted by EtOAc. Obtained organic layer was sequentially washed with aqueous sodium thiosulfate, water and brine. Collected organic layer was dried over Na₂SO₄, filtered and concentrated to obtain brownish crude, which was purified by CombiFlash column chromatography using 20-40% EtOAc-hexane gradient to afford 4-bromo-5-iodo-3-(1-phenylvinyl)pyridin-2-amine (6) (760 mg, 1.33 mmol, 70% purity, 36% yield) as light brown solid.

LCMS: (ES⁺) m/z (M+H)⁺=400.5, Rt=3.63 min.

### Step 5: 3-((4-bromo-5-iodo-3-(1-phenylvinyl)pyridin-2-yl)amino)-5,5-dimethylcyclohex-2-en-1-one. (8)

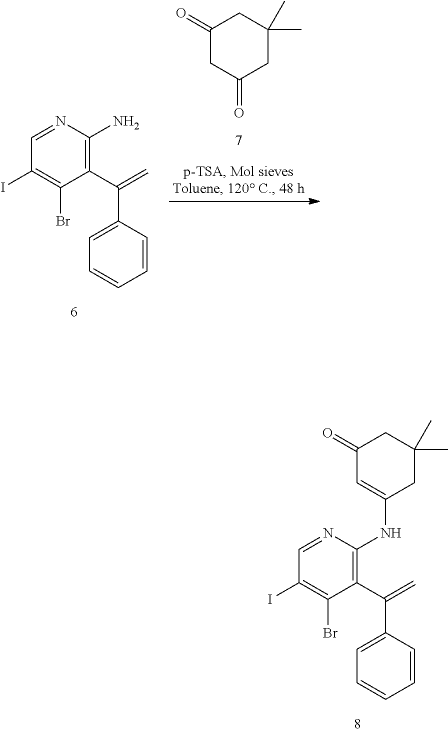

To a stirred solution of 4-bromo-5-iodo-3-(1-phenylvinyl)pyridin-2-amine (6) (2.50 g, 6.23 mmol, 1.00 eq) and 5,5-dimethylcyclohexane-1,3-dione (7) (1 g, 7.48 mmol, 1.20 eq) in toluene (25 mL) were sequentially added p-TSA monohydrate (118 mg, 0.62 mmol, 0.1 eq) and dried molecular sieves (200 mg) at RT under inert atmosphere. The resulting mixture was allowed to stir at 120° C. for 48 h. On completion, the reaction mixture was filtered through celite pad and washed with EtOAc. Obtained filtrate was evaporated under reduced pressure to furnish a brownish crude. Crude was purified by CombiFlash chromatography using 25-50% EtOAc-hexane gradient to afford 3-[[4-bromo-5-iodo-3-(1-phenylvinyl)-2-pyridyl]amino]-5,5-dimethyl-cyclohex-2-en-1-one (8) (1.8 g, 3.27 mmol, 95% purity, 55% yield).

LCMS: (ES⁺) m/z (M+H)⁺=524.8, Rt=3.81 min.

### Step 6: 4-bromo-3-iodo-5,8,8-trimethyl-5-phenyl-5,8,9,10-tetrahydrobenzo[b][1,8] naphthyridine-6(7H)-one (9)

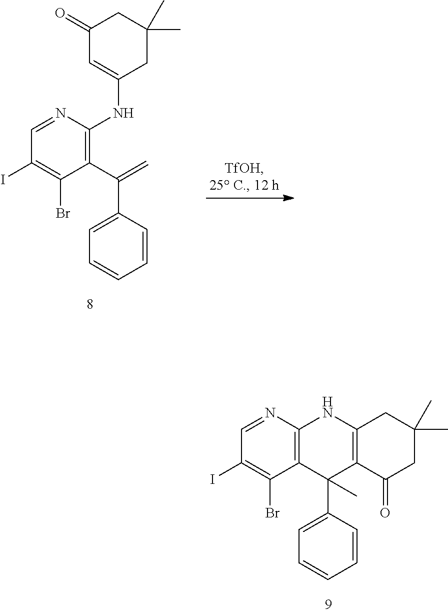 [SMILES: C=C(c1ccccc1)c1c(NC2=CC(=O)CC(C)(C)C2)ncc(I)c1Br ; CC1(C)CC(=O)C2=C(C1)Nc1ncc(I)c(Br)c1C2(C)c1ccccc1]

TfOH (35 mL) was portion wise added to a RB flask containing 3-[[4-bromo-5-iodo-3-(1-phenylvinyl)-2-pyridyl]amino]-5,5-dimethyl-cyclohex-2-en-1-one (8) (3.5 g, 6.69 mmol, 1 eq) under inert atmosphere at 0° C. Resulting mixture was allowed to stir at 25° C. for 12 h. On completion, the reaction mixture was quenched with aqueous NaHCO₃ solution and extracted with EtOAc. Obtained organic layer washed with water, brine, dried over Na₂SO₄, filtered and concentrated to obtain a brownish crude. Crude was purified by CombiFlash column chromatography using 20-60% EtOAc-hexane gradient to afford 4-bromo-3-iodo-5,8,8-trimethyl-5-phenyl-9,10-dihydro-7H-benzo[b][1,8]naphthyridin-6-one (9) (2.9 g, 4.99 mmol, 90% purity, 75% yield) as off white solid.

LCMS: (ES⁺) m/z (M+H)⁺=523.0, Rt=3.71 min.

### Step 7: 4-bromo-5,8,8-trimethyl-5-phenyl-3-vinyl-5,8,9,10-tetrahydrobenzo[b][1,8] naphthyridin-6(7H)-one (11)

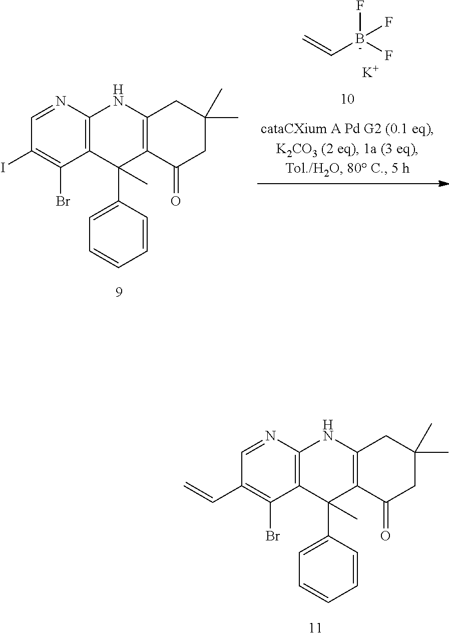

To a stirred degassed solution of 4-bromo-3-iodo-5,8,8-trimethyl-5-phenyl-9,10-dihydro-7H-benzo[b][1,8]naphthyridin-6-one (9) (500 mg, 0.96 mmol, 1 eq) in 4:1 toluene-water mixture (12 mL) were sequentially added potassium trifluoro(vinyl)boranuide (384 mg, 2.87 mmol, 3.00 eq), K₂CO₃ (264 mg, 1.91 mmol, 2.00 eq) and cataCXium A Pd G2 (65 mg, 0.09 mmol, 0.1 eq). The resulting mixture was allowed to stir at 80° C. for 5 h. Reaction mixture was diluted with EtOAc, washed with water and brine. Organic layer was collected, dried over Na₂SO₄, filtered and concentrated under reduced pressure to afford a brownish crude. Crude was purified by CombiFlash column chromatography using 15-40% EtOAc-hexane gradient to afford 4-bromo-5,8,8-trimethyl-5-phenyl-3-vinyl-9,10-dihydro-7H-benzo[b][1,8]naphthyridine-6-one (11) (190 mg, 0.408 mmol, 91% purity, 43% yield) as off white solid.

LCMS: (ES⁺) m/z (M+H)⁺=423.0, Rt=3.66 min.

### Step 8: 5,8,8-trimethyl-6-oxo-5-phenyl-3-vinyl-5,6,7,8,9,10-hexahydrobenzo[b][1,8] naphthyridine-4-carbonitrile (12)

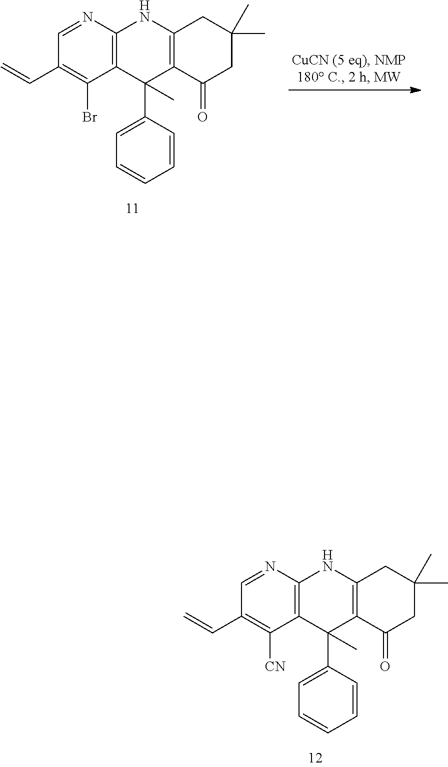 [SMILES: C=Cc1cnc2c(c1Br)C(C)(c1ccccc1)C1=C(CC(C)(C)CC1=O)N2 ; C=Cc1cnc2c(c1C#N)C(C)(c1ccccc1)C1=C(CC(C)(C)CC1=O)N2]

NMP (3 mL) and CuCN (159 mg, 1.77 mmol, 5 eq) were sequentially added to a MW vial charged with 4-bromo-5,8,8-trimethyl-5-phenyl-3-vinyl-9,10-dihydro-7H-benzo[b][1,8] naphthyridin-6-one (11) (150 mg, 0.354 mmol, 1 eq). The resulting mixture was allowed to stir at 180° C. for 2 h under microwave irradiation. On completion, the reaction mixture was diluted with EtOAc and washed sequentially with water and brine, dried over sodium sulfate, filtered and concentrated under reduced pressure to afford a yellowish crude. Obtained crude was purified by CombiFlash column chromatograph using EtOAc in Hexane to afford 5,8,8-trimethyl-6-oxo-5-phenyl-3-vinyl-9,10-dihydro-7H-benzo[b][1,8]naphthyridine-4-carbonitrile (12) (55 mg, 0.149 mmol, 88% purity, 42% yield) as off white solid.

LCMS: (ES⁺) m/z (M+H)⁺=370.0, Rt=1.97 min.

### Step 9: 3-formyl-5,8,8-trimethyl-6-oxo-5-phenyl-5,6,7,8,9,10-hexahydrobenzo[b][1,8] naphthyridine-4-carbonitrile (13)

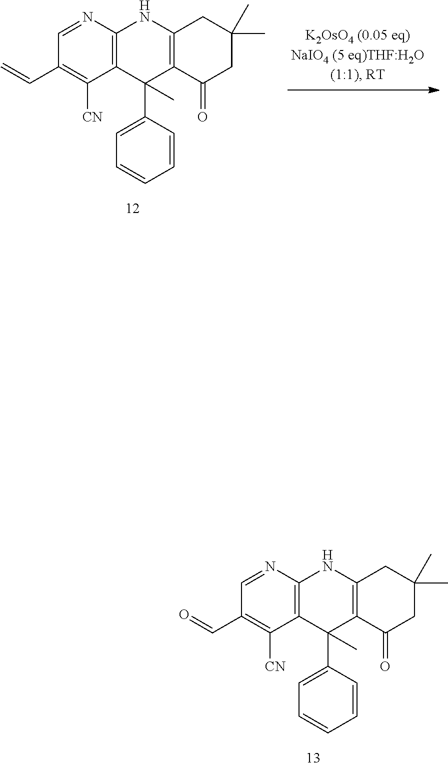 [SMILES: C=Cc1cnc2c(c1C#N)C(C)(c1ccccc1)C1=C(CC(C)(C)CC1=O)N2 ; CC1(C)CC(=O)C2=C(C1)Nc1ncc(C=O)c(C#N)c1C2(C)c1ccccc1]

To a stirred solution of 5,8,8-trimethyl-6-oxo-5-phenyl-3-vinyl-9,10-dihydro-7H-benzo[b][1,8]naphthyridine-4-carbonitrile (12) (245 mg, 0.66 mmol, 1 eq) in 1:1 THF-H₂O mixture (8 mL) were sequentially added potassium osmate (11 mg, 0.03 mmol, 0.05 eq) and sodium periodate (709 mg, 3.32 mmol, 5 eq). The resulting mixture was allowed to stir at RT for 3 h. On completion, the reaction mixture was diluted with water and extracted with ethyl acetate. Obtained organic layer was washed sequentially with cold water and brine, dried over Na₂SO₄, filtered and concentrated under reduced pressure to afford crude 3-formyl-5,8,8-trimethyl-6-oxo-5-phenyl-9,10-dihydro-7H-benzo[b][1,8]naphthyridine-4-carbonitrile (13) (220 mg, 0.59 mmol, 92% purity, 89% yield) as yellow solid. Obtained crude was used as such for the next step without further purification.

LCMS: (ES⁺) m/z (M+H)⁺=372.2, Rt=3.24 min.

### Step 10: 3-(difluoromethyl)-5,8,8-trimethyl-6-oxo-5-phenyl-5,6,7,8,9,10-hexahydrobenzo [b][1,8]naphthyridine-4-carbonitrile (14)

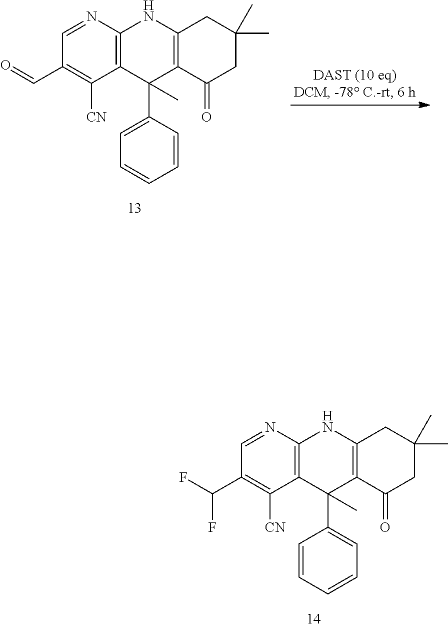 [SMILES: CC1(C)CC(=O)C2=C(C1)Nc1ncc(C=O)c(C#N)c1C2(C)c1ccccc1 ; CC1(C)CC(=O)C2=C(C1)Nc1ncc(C(F)F)c(C#N)c1C2(C)c1ccccc1]

DAST (1.96 mL, 14.8 mmol, 10 eq) was dropwise added to a stirred solution of 3-formyl-5,8,8-trimethyl-6-oxo-5-phenyl-9,10-dihydro-7H-benzo[b][1,8]naphthyridine-4-carbonitrile (13) (550 mg, 1.48 mmol, 1 eq) in DCM (8 mL) at −78° C. under inert atmosphere. The resulting mixture was allowed to stir under same condition for 30 min and then allowed to stir at 25° C. for another 6 h. On completion, the reaction mixture was diluted with DCM and quenched with aqueous sodium bicarbonate solution. The obtained organic layer was washed with water, brine, dried over Na₂SO₄, filtered and concentrated under reduced pressure to afford a yellowish crude. Obtained crude was purified by CombiFlash column chromatography using 10-30% EtOAc-hexane gradient to afford 3-(difluoromethyl)-5,8,8-trimethyl-6-oxo-5-phenyl-9,10-dihydro-7H-benzo[b][1,8]naphthyridine-4-carbonitrile (14) (210 mg, 0.5 mmol, 93% purity, 34% yield) as yellow solid.

LCMS: (ES⁺) m/z (M+H)⁺=394.2, Rt=2.80 min.

### Step 11: Chiral Separation

### (S)-3-(difluoromethyl)-5,8,8-trimethyl-6-oxo-5-phenyl-5,6,7,8,9,10-hexahydrobenzo[b][1,8] naphthyridine-4-carbonitrile (14A)

### (R)-3-(difluoromethyl)-5,8,8-trimethyl-6-oxo-5-phenyl-5,6,7,8,9,10-hexahydrobenzo[b][1,8] naphthyridine-4-carbonitrile (14B)

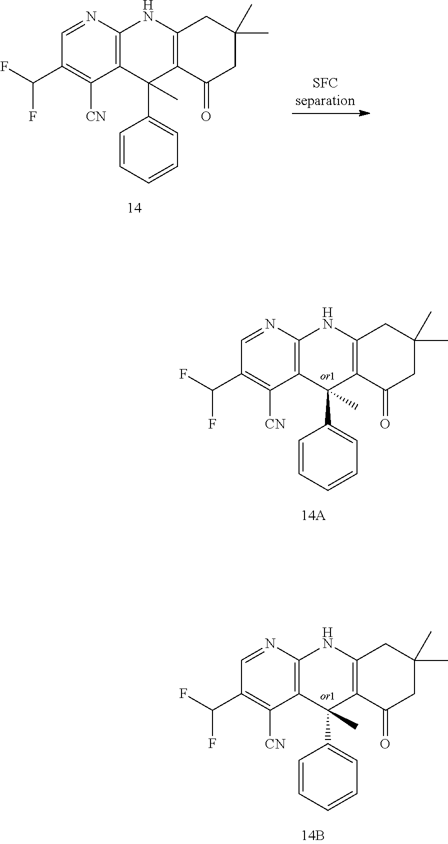 [SMILES: CC1(C)CC(=O)C2=C(C1)Nc1ncc(C(F)F)c(C#N)c1C2(C)c1ccccc1 ; CC1(C)CC(=O)C2=C(C1)Nc1ncc(C(F)F)c(C#N)c1[C@]2(C)c1ccccc1 ; CC1(C)CC(=O)C2=C(C1)Nc1ncc(C(F)F)c(C#N)c1[C@@]2(C)c1ccccc1]

Chiral separation of 3-(difluoromethyl)-5,8,8-trimethyl-6-oxo-5-phenyl-9,10-dihydro-7H-benzo[b][1,8]naphthyridine-4-carbonitrile (14) (210 mg, 0.5 mmol) was done by prep SFC on Waters SFC 150 instrument equipped with Waters 2489 UV/Visible Detector by using (Column: (R,R) Whelk-O1 (21.1 mm×250 mm)), 5μ Column operating at 35° C. temperature, maintaining flow rate of 70 ml/min, using 50% CO₂ in super critical state & 50% of [100% Methanol] as Mobile phase. Run this isocratic mixture up to 10.0 minutes and also maintained the isobaric condition of 100 bar at 244 nm wavelength.

SFC fractions were collected and evaporated under reduced pressure to afford:

Peak 1: (S)-3-(difluoromethyl)-5,8,8-trimethyl-6-oxo-5-phenyl-5,6,7,8,9,10-hexahydrobenzo [b][1,8] naphthyridine-4-carbonitrile (14) (94 mg, 0.236 mmol, 98.89% purity, 30% yield)

UPLC: (ES⁺) m/z (M+H)⁺=394.2, Rt=2.43 min.

HPLC: 98.89%

¹H NMR (400 MHz, DMSO-d6) δ 10.48 (s, 1H), 8.43 (s, 1H), 7.34 (d, 2H), 7.22 (t, 2H), 7.15-6.88 (m, 2H), 2.46 (s, 2H), 2.17 (s, 3H), 2.01 (d, 1H), 1.89 (d, 1H), 0.99 (s, 3H), 0.91 (s, 3H).

[α]²⁵=313.854°, c=0.1284, MeOH.

Chiral purity (ee %): 100

Peak 2: (R)-3-(difluoromethyl)-5,8,8-trimethyl-6-oxo-5-phenyl-5,6,7,8,9,10-hexahydrobenzo [b][1,8] naphthyridine-4-carbonitrile (14B) (91 mg, 0.227 mmol, 98.14% purity, 29% yield)

UPLC: (ES⁺) m/z (M+H)⁺=394.3, Rt=2.45 min.

¹H NMR (400 MHz, DMSO-d6) d 10.48 (s, 1H), 8.43 (s, 1H), 7.34 (d, 2H), 7.22 (t, 2H), 7.15-6.88 (m, 2H), 2.46 (s, 2H), 2.17 (s, 3H), 2.01 (d, 1H), 1.89 (d, 1H), 0.99 (s, 3H), 0.91 (s, 3H).

[α]²⁵=−305.731, c=0.1308, MeOH.

Chiral purity (ee %): 99.52

### Example 3.2. (S)-3-(fluoromethyl)-5,8,8-trimethyl-6-oxo-5-phenyl-5,6,7,8,9,10-hexahydrobenzo[b][1,8]naphthyridine-4-carbonitrile (3A) and (S)-3-(fluoromethyl)-5,8,8-trimethyl-6-oxo-5-phenyl-5,6,7,8,9,10-hexahydrobenzo[b][1,8]naphthyridine-4-carbonitrile (3B)

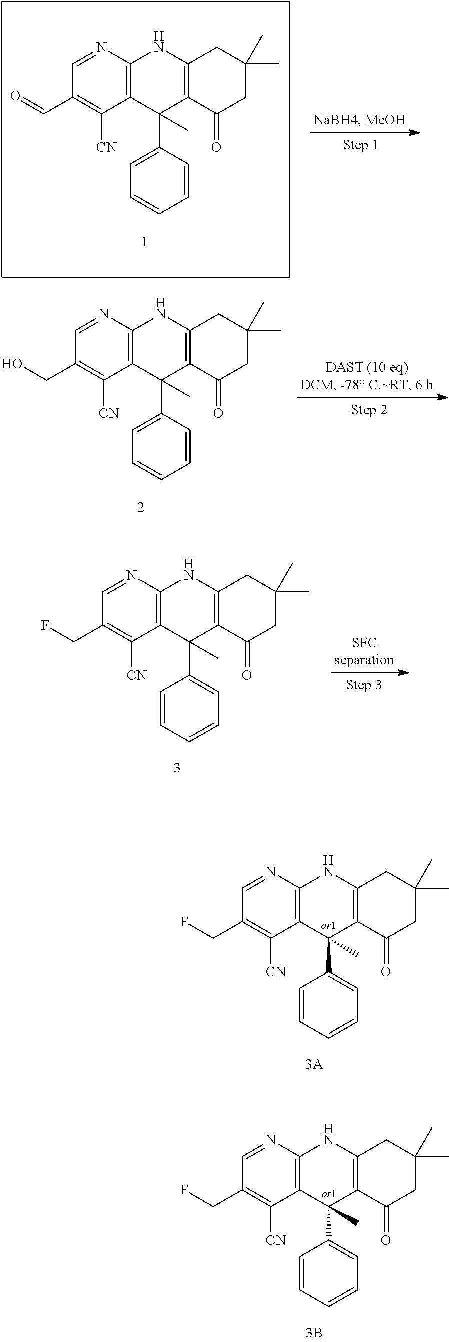 [SMILES: CC1(C)CC(=O)C2=C(C1)Nc1ncc(C=O)c(C#N)c1C2(C)c1ccccc1 ; CC1(C)CC(=O)C2=C(C1)Nc1ncc(CO)c(C#N)c1C2(C)c1ccccc1 ; CC1(C)CC(=O)C2=C(C1)Nc1ncc(CF)c(C#N)c1C2(C)c1ccccc1 ; CC1(C)CC(=O)C2=C(C1)Nc1ncc(CF)c(C#N)c1[C@]2(C)c1ccccc1 ; CC1(C)CC(=O)C2=C(C1)Nc1ncc(CF)c(C#N)c1[C@@]2(C)c1ccccc1]

### Step 1: 3-(hydroxymethyl)-5,8,8-trimethyl-6-oxo-5-phenyl-5,6,7,8,9,10-hexahydrobenzo[b] [1,8]naphthyridine-4-carbonitrile (2)

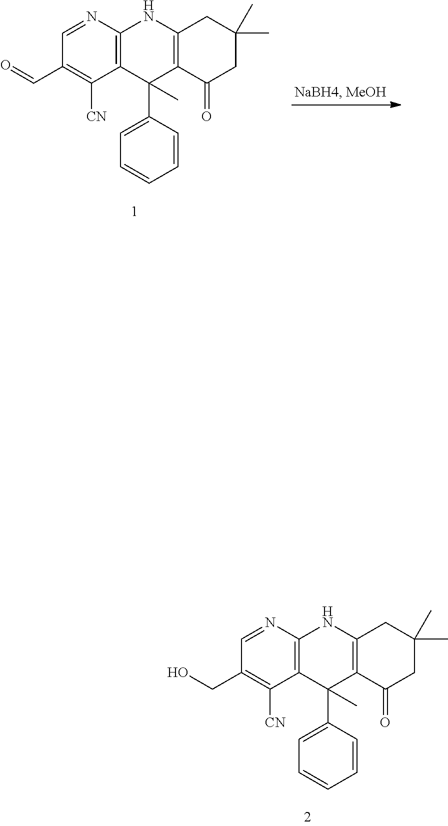 [SMILES: CC1(C)CC(=O)C2=C(C1)Nc1ncc(C=O)c(C#N)c1C2(C)c1ccccc1 ; CC1(C)CC(=O)C2=C(C1)Nc1ncc(CO)c(C#N)c1C2(C)c1ccccc1]

To a stirred solution of 3-formyl-5,8,8-trimethyl-6-oxo-5-phenyl-9,10-dihydro-7H-benzo[b][1,8]naphthyridine-4-carbonitrile (1) (230 mg, 0.62 mmol, 1 eq) in MeOH (5 mL), was portion wise added NaBH4 (70 mg, 1.86 mmol, 3.00 eq) under ice cool condition. The resulting mixture was allowed to stir at 25° C. for 3 h. On completion, the reaction mixture was diluted with DCM, quenched with aqueous NaHCO₃ solution. Organic layer was collected, dried over Na₂SO₄, filtered and concentrated to afford a colorless crude, which was purified by CombiFlash column chromatography using 20-60% EtOAc-hexane gradient to furnish 3-(hydroxymethyl)-5,8,8-trimethyl-6-oxo-5-phenyl-9,10-dihydro-7H-benzo[b][1,8]naphthyridine-4-carbonitrile (2) (110 mg, 0.29 mmol, 98% purity, 47% yield) as light yellow solid.

LCMS: (ES⁺) m/z (M+H)⁺=374.3, Rt=2.31 min.

### Step 2: 3-(fluoromethyl)-5,8,8-trimethyl-6-oxo-5-phenyl-5,6,7,8,9,10-hexahydrobenzo[b] [1,8]naphthyridine-4-carbonitrile (3)

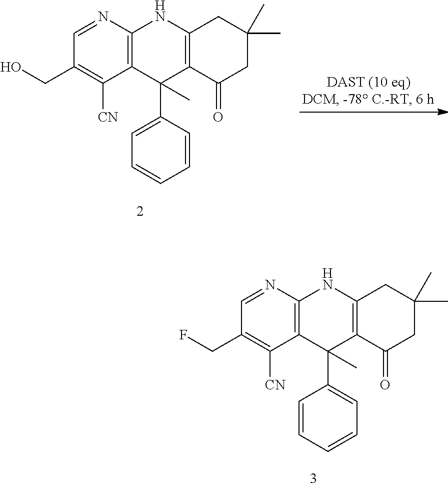 [SMILES: CC1(C)CC(=O)C2=C(C1)Nc1ncc(CO)c(C#N)c1C2(C)c1ccccc1 ; CC1(C)CC(=O)C2=C(C1)Nc1ncc(CF)c(C#N)c1C2(C)c1ccccc1]

To a stirred solution of 3-(hydroxymethyl)-5,8,8-trimethyl-6-oxo-5-phenyl-9,10-dihydro-7H-benzo[b][1,8]naphthyridine-4-carbonitrile (2) (120 mg, 0.32 mmol, 1 eq) in DCM (5 mL) was dropwise added DAST (0.21 mL, 1.61 mmol, 5.00 eq) at −78° C. under inert atmosphere. The resulting mixture was allowed to stir for 30 min under the same temperature and then at 25° C. for an additional 6 h. On completion, reaction mixture was diluted with DCM, quenched with NaHCO₃ solution. The organic layer was collected, dried over Na₂SO₄, filtered and concentrated to obtain a crude yellowish crude. Crude was purified by CombiFlash column chromatography using 10-30% EtOAc in hexane gradient to afford 3-(fluoromethyl)-5,8,8-trimethyl-6-oxo-5-phenyl-9,10-dihydro-7H-benzo[b][1,8]naphthyridine-4-carbonitrile (3) (45 mg, 0.119 mmol, 99% purity, 37% yield) as light yellow solid.

LCMS: (ES⁺) m/z (M+H)⁺=376.2, Rt=3.62 min.

### Step 3: Chiral Separation

### (S)-3-(fluoromethyl)-5,8,8-trimethyl-6-oxo-5-phenyl-5,6,7,8,9,10-hexahydrobenzo[b][1,8] naphthyridine-4-carbonitrile (3A)

### (R)-3-(fluoromethyl)-5,8,8-trimethyl-6-oxo-5-phenyl-5,6,7,8,9,10-hexahydrobenzo[b][1,8] naphthyridine-4-carbonitrile (3B)

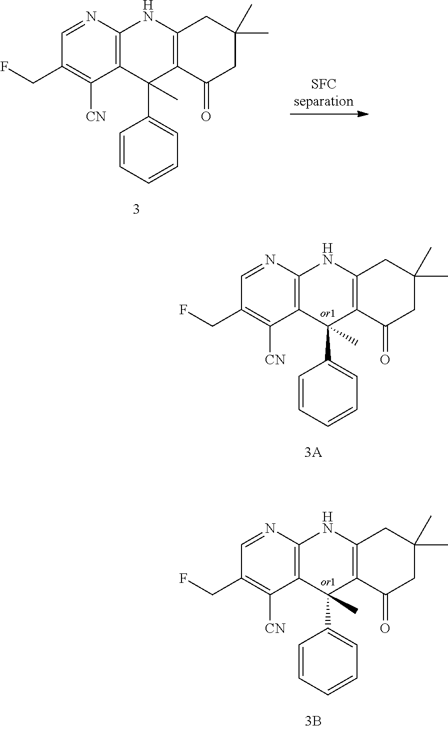 [SMILES: CC1(C)CC(=O)C2=C(C1)Nc1ncc(CF)c(C#N)c1C2(C)c1ccccc1 ; CC1(C)CC(=O)C2=C(C1)Nc1ncc(CF)c(C#N)c1[C@]2(C)c1ccccc1 ; CC1(C)CC(=O)C2=C(C1)Nc1ncc(CF)c(C#N)c1[C@@]2(C)c1ccccc1]

Chiral separation of 3-(fluoromethyl)-5,8,8-trimethyl-6-oxo-5-phenyl-9,10-dihydro-7H-benzo[b][1,8]naphthyridine-4-carbonitrile (3) (45 mg, 0.119 mmol) was done by prep SFC-80 instrument equipped with waters 2489 UV/VISIBLE detector, by using (R,R) Whelk-O1 column (21.1 mm×250 mm), 5μ Column operating at 35° C. temperature, maintaining flow rate of 70 ml/min using 60% CO2 in super critical state & 40%[100% MeOH] as Mobile phase. Run this isocratic mixture up to 10.0 minutes and also maintained the isobaric condition of 100 bar at 220 nm wavelength.

SFC fractions were collected and evaporated under reduced pressure to afford:

Peak 1: (S)-3-(fluoromethyl)-5,8,8-trimethyl-6-oxo-5-phenyl-5,6,7,8,9,10-hexahydrobenzo[b][1,8]naphthyridine-4-carbonitrile (3A) (7.9 mg, 0.0194 mmol, 91.82% purity, 16% yield).

UPLC: (ES⁺) m/z (M+H)⁺=376.3, Rt=2.38 min.

¹H NMR (400 MHz, DMSO-d6) δ 10.33 (s, 1H), 8.38 (s, 1H), 7.34 (d, 2H), 7.21 (t, 2H), 7.10 (t, 11H), 5.37 (s, 1H), 5.28 (s, 11H), 2.46 (s, 2H), 2.16 (s, 3H), 2.01 (d, 1H), 1.88 (d, 1H), 0.99 (s, 3H), 0.91 (s, 3H).

[α]²⁵=+137.507, c=0.1018, CH3CN.

Chiral purity (ee %): 100

Peak 2: (R)-3-(fluoromethyl)-5,8,8-trimethyl-6-oxo-5-phenyl-5,6,7,8,9,10-hexahydrobenzo[b][1,8] naphthyridine-4-carbonitrile (3B) (7.3 mg, 0.0181 mmol, 92.98% purity, 15% yield).

UPLC: (ES⁺) m/z (M+H)⁺=376.3, Rt=2.36 min.

¹H NMR (400 MHz, DMSO-d6) δ 10.33 (s, 1H), 8.38 (s, 1H), 7.34 (d, 2H), 7.21 (t, 2H), 7.08 (t, 1H), 5.37 (s, 1H), 5.25 (s, 1H), 2.46 (s, 2H), 2.16 (s, 3H), 2.01 (d, 1H), 1.88 (d, 1H), 0.99 (s, 3H), 0.91 (s, 3H).

[α]²⁵=−154.204, c=0.1018, CH3CN.

Chiral purity (ee %): 100

### Example 3.3. (S)-3-(difluoromethyl)-5,8,8-trimethyl-6-oxo-5-phenyl-5,6,7,8,9,10-hexahydropyrido[2,3-b][1,6]naphthyridine-4-carbonitrile (9A) and (R)-3-(difluoromethyl)-5,8,8-trimethyl-6-oxo-5-phenyl-5,6,7,8,9,10-hexahydropyrido[2,3-b][1,6]naphthyridine-4-carbonitrile (9B)

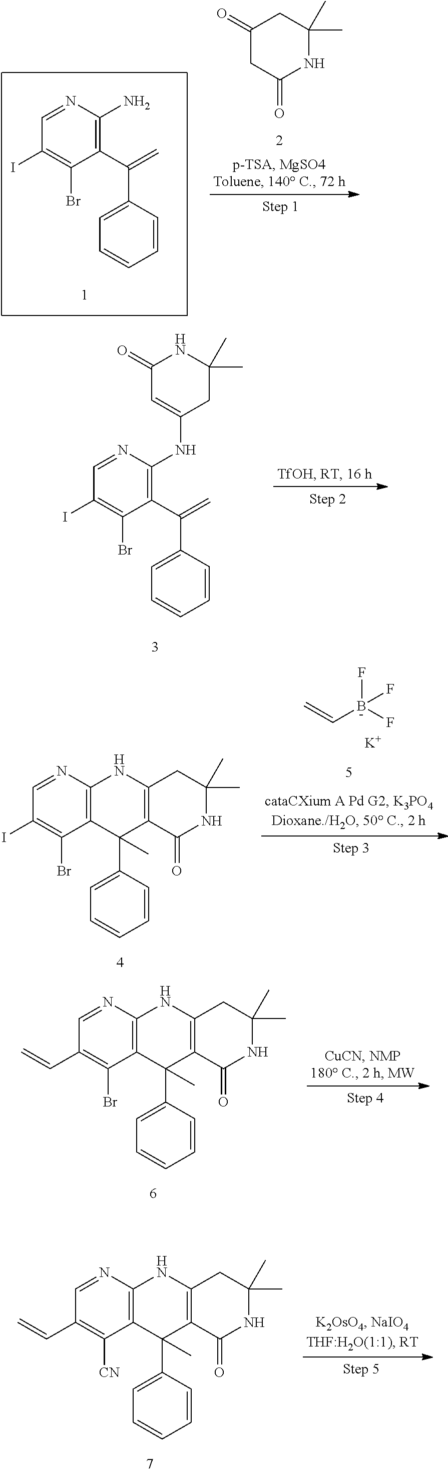

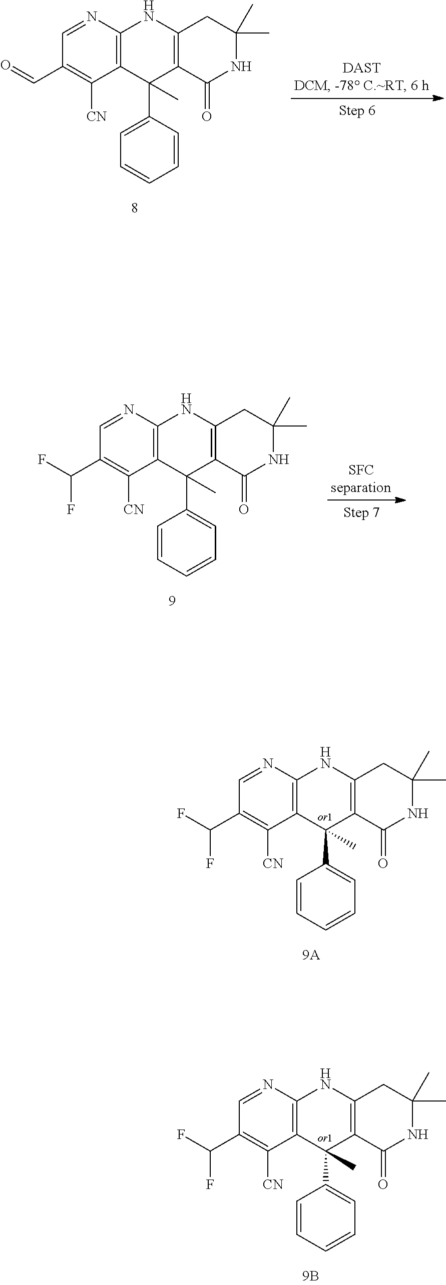 [SMILES: CC1(C)CC2=C(C(=O)N1)C(C)(c1ccccc1)c1c(ncc(C=O)c1C#N)N2 ; CC1(C)CC2=C(C(=O)N1)C(C)(c1ccccc1)c1c(ncc(C(F)F)c1C#N)N2 ; CC1(C)CC2=C(C(=O)N1)[C@@](C)(c1ccccc1)c1c(ncc(C(F)F)c1C#N)N2 ; CC1(C)CC2=C(C(=O)N1)[C@](C)(c1ccccc1)c1c(ncc(C(F)F)c1C#N)N2]

### Step 1: 4-((4-bromo-5-iodo-3-(I-phenylvinyl) pyridin-2-yl) amino)-6,6-dimethyl-5,6-dihydropyridin-2(1H)-one (3)

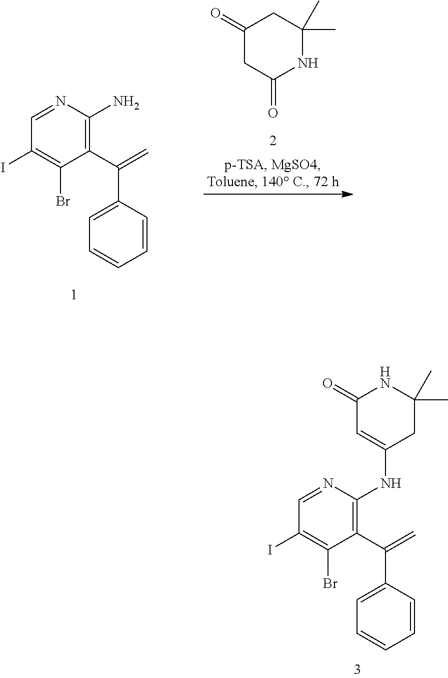

To a stirred solution of 4-bromo-5-iodo-3-(1-phenylvinyl) pyridin-2-amine (1) (2 g, 5 mmol, 1 eq) in Toluene (80 mL) were sequentially added 6,6-dimethylpiperidine-2,4-dione (2) (1.05 g, 7.48 mmol, 1.5 eq), p-Toluene sulfonic acid monohydrate (190 mg, 1 mmol, 0.2 eq) and MgSO₄ (2.39 g, 19.9 mmol, 4 eq) under inert atmosphere at RT. Reaction mixture was stirred at 140° C. for 72 h. On completion, reaction mixture was filtered through celite pad and the obtained filtrate was evaporated under reduce pressure to get a brownish crude. Obtained crude was purified by CombiFlash column chromatography using 2% MeOH in DCM to provide 4-[[4-bromo-5-iodo-3-(1-phenylvinyl)-2-pyridyl]amino]-2,2-dimethyl-1,3-dihydropyridin-6-one (3) (1.10 g, 1.65 mmol, 78.47% purity, 33% yield) as off white solid.

LCMS: (ES⁺) m/z (M+H)⁺=524.1, Rt=3.43 min.

### Step 2: 4-bromo-3-iodo-5,8,8-trimethyl-5-phenyl-5,8,9,10-tetrahydropyrido[2,3-b][1,6]naphthyridin-6(7H)-one. (4)

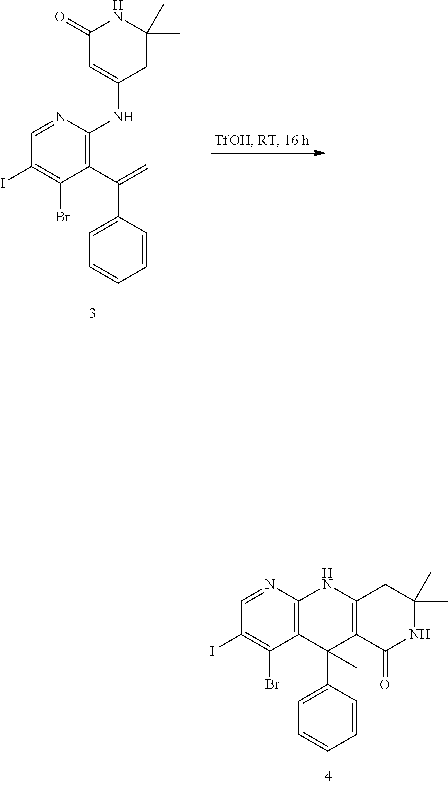 [SMILES: C=C(c1ccccc1)c1c(NC2=CC(=O)NC(C)(C)C2)ncc(I)c1Br ; CC1(C)CC2=C(C(=O)N1)C(C)(c1ccccc1)c1c(ncc(I)c1Br)N2]

TfOH (10 mL) was dropwise added to RB flask, containing 4-[4-bromo-5-iodo-3-(1-phenylvinyl)-2-pyridyl]amino]-2,2-dimethyl-1,3-dihydropyridin-6-one (3) (1.10 g, 2.10 mmol, 1 eq) at 0° C. under inert atmosphere. Reaction mixture was stirred at RT for 16 h. On completion, reaction mixture was poured slowly to the aqueous NaHCO₃ (1M) solution at 0° C., extracted with EtOAc (3×100 mL). The combined organic layer was washed with aqueous NaHCO₃ (1 M) solution. Organic layer was dried over Na₂SO₄, filtered and concentrated under reduced pressure to get crude product which was purified by CombiFlash column chromatography using 50% EtOAc in hexane to provide 4-bromo-3-iodo-5,8,8-trimethyl-5-phenyl-9,10-dihydro-7H-pyrido[2,3-b][1,6]naphthyridin-6-one (4) (670 mg, 1.28 mmol, 95% purity, 58% yield).

LCMS: (ES⁺) m/z (M+H)⁺=523.8, Rt=3.43 min.

### Step 3: 4-bromo-5,8,8-trimethyl-5-phenyl-3-vinyl-5,8,9,10-tetrahydropyrido[2,3-b][1,6]naphthyridin-6(7H)-one. (6)

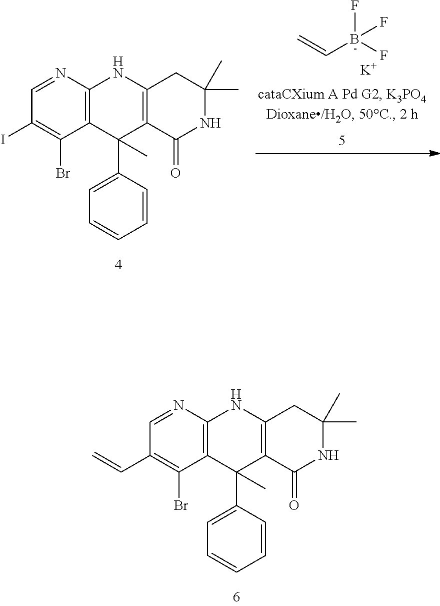

To a stirred solution of 4-bromo-3-iodo-5,8,8-trimethyl-5-phenyl-9,10-dihydro-7H-pyrido[2,3-b][1,6]naphthyridin-6-one (4) (630 mg, 1.20 mmol, 1.00 eq) in dioxane-water (4:1) (30 mL) were sequentially added potassium trifluoro(vinyl)boranuide (177 mg, 1.32 mmol, 1.10 eq) and K₃PO₄ (510 mg, 2.40 mmol, 2 eq). Reaction mixture was purged with argon for 10 mins followed by addition of cataCXium A Pd G2 (80 mg, 0.120 mmol, 0.1 eq). The resulting mixture was allowed to stir at 50° C. for 2 h. Reaction mixture was diluted with water (10 mL) and extracted with EtOAc (3×50 mL). The combined organic layer was washed with brine. Organic layer was dried over Na2SO4, filtered and concentrated under reduced pressure to get crude product which was purified by CombiFlash column chromatography using 40% EtOAc in hexane to provide 4-bromo-5,8,8-trimethyl-5-phenyl-3-vinyl-5,8,9,10-tetrahydropyrido[2,3-b][1,6]naphthyridin-6(7H)-one (6) (210 mg, 0.466 mmol, 94.16% purity, 39% yield) as colorless sticky gum.

LCMS: (ES⁺) m/z (M+H)⁺=424.0, Rt=3.39 min.

### Step 4: 5,8,8-trimethyl-6-oxo-5-phenyl-3-vinyl-5,6,7,8,9,10-hexahydropyrido[2,3-b][1,6] naphthyridine-4-carbonitrile. (7)

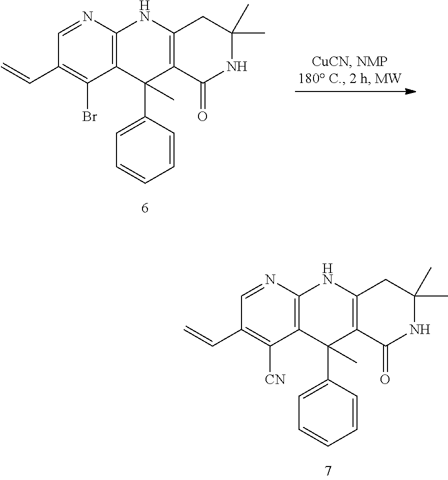 [SMILES: C=Cc1cnc2c(c1Br)C(C)(c1ccccc1)C1=C(CC(C)(C)NC1=O)N2 ; C=Cc1cnc2c(c1C#N)C(C)(c1ccccc1)C1=C(CC(C)(C)NC1=O)N2]

To a stirred solution of 4-bromo-5,8,8-trimethyl-5-phenyl-3-vinyl-9,10-dihydro-7H-pyrido[2,3-b][1,6]naphthyridin-6-one (6) (210 mg, 0.495 mmol, 1.00 eq) in NMP (2 mL) taken in microwave vial, was added CuCN (221 mg, 2.47 mmol, 5 eq). Reaction mixture was allowed to stir at 180° C. for 2 h under microwave irradiation. On completion, the reaction mixture was diluted with water (3 mL) and extracted with EtOAc (3×25 mL). Combined organic layer was washed with brine, dried over Na₂SO₄, filtered and concentrated under reduced pressure to get crude product which was purified by CombiFlash column chromatography using 40-50% EtOAc in DCM gradient to provide 5,8,8-trimethyl-6-oxo-5-phenyl-3-vinyl-5,6,7,8,9,10-hexahydropyrido[2,3-b]11.61 naphthyridine-4-carbonitrile (7) (50 mg, 0.135 mmol, 95% purity, 27% yield) as colorless solid.

LCMS: (ES⁺) m/z (M+H)⁺=370.9, Rt=3.12 min.

### Step 5: 3-formyl-5,8,8-trimethyl-6-oxo-5-phenyl-5,6,7,8,9,10-hexahydropyrido[2,3-b][1,6]naphthyridine-4-carbonitrile. (8)

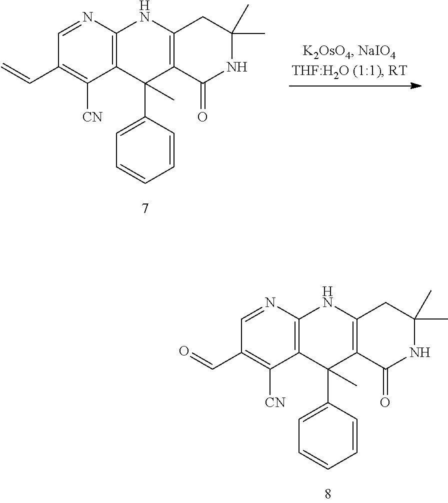 [SMILES: C=Cc1cnc2c(c1C#N)C(C)(c1ccccc1)C1=C(CC(C)(C)NC1=O)N2 ; CC1(C)CC2=C(C(=O)N1)C(C)(c1ccccc1)c1c(ncc(C=O)c1C#N)N2]

To a stirred solution of 5,8,8-trimethyl-6-oxo-5-phenyl-3-vinyl-9,10-dihydro-7H-pyrido[2,3-b][1,6]naphthyridine-4-carbonitrile (7) (40 mg, 0.108 mmol, 1.00 eq) in THF-Water (1:1) (4 mL) were added potassium osmate (1.8 mg, 0.005 mmol, 0.05 eq) and sodium periodate (115 mg, 0.54 mmol, 5 eq) at RT. Reaction mixture was stirred at RT for 3 h. On completion, the reaction mixture was diluted with water and extracted with EtOAc (twice). The combined organic layer was washed with brine, dried over Na₂SO₄, filtered and concentrated under reduced pressure to provide crude 3-formyl-5,8,8-trimethyl-6-oxo-5-phenyl-5,6,7,8,9,10-hexahydropyrido[2,3-b][1,6]naphthyridine-4-carbonitrile (30 mg, 0.06 mmol, 80% purity, 60% yield) as colorless sticky gum.

LCMS: (ES⁺) m/z (M+H)⁺=373.3, Rt=2.88 min.

### Step 6: 3-(difluoromethyl)-5,8,8-trimethyl-6-oxo-5-phenyl-5,6,7,8,9,10-hexahydropyrido[2,3-b][1,6]naphthyridine-4-carbonitrile. (9)

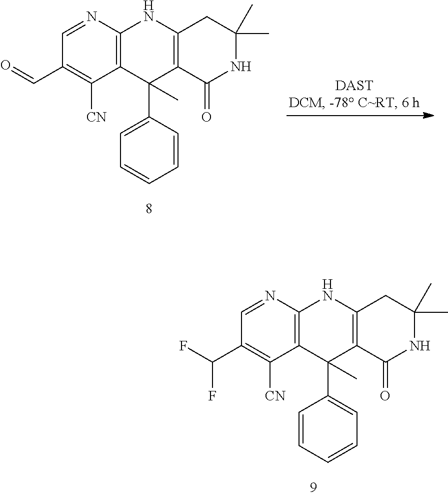 [SMILES: CC1(C)CC2=C(C(=O)N1)C(C)(c1ccccc1)c1c(ncc(C=O)c1C#N)N2 ; CC1(C)CC2=C(C(=O)N1)C(C)(c1ccccc1)c1c(ncc(C(F)F)c1C#N)N2]

To a stirred solution of 3-formyl-5,8,8-trimethyl-6-oxo-5-phenyl-9,10-dihydro-7H-pyrido[2,3-b] [1,6]naphthyridine-4-carbonitrile (8) (100 mg, 0.269 mmol, 1.00 eq) in DCM (3 mL) was added DAST (0.35 mL, 2.69 mmol, 10.0 eq) at −78° C. Reaction mixture was stirred at −78° C. for 30 min. Then the reaction mixture was warmed up to RT and stirred for another 6 h. On completion, reaction mixture was quenched with aqueous NaHCO₃ (1M; 2 mL) and extracted with DCM (2×25 mL). The combined organic layer was washed with brine, dried over Na₂SO₄, filtered and concentrated under reduced pressure to get a brownish crude. Obtained crude was purified by CombiFlash column chromatography using 30% EtOAc in hexane to provide 3-(difluoromethyl)-5,8,8-trimethyl-6-oxo-5-phenyl-5,6,7,8,9,10-hexahydropyrido[2,3-b][1,6]naphthyridine-4-carbonitrile (9) (20 mg, 0.05 mmol, 50% purity, 9% yield) as yellow solid.

LCMS: (ES⁺) m/z (M+H)⁺=395.2, Rt=2.12 min.

Note: LCMS showed ˜50% peak having 25 mass unit less than desired mass.

### Step 7: Chiral Separation

### (S)-3-(difluoromethyl)-5,8,8-trimethyl-6-oxo-5-phenyl-5,6,7,8,9,10-hexahydropyrido[2,3-b][1,6]naphthyridine-4-carbonitrile (9A) &

### (R)-3-(difluoromethyl)-5,8,8-trimethyl-6-oxo-5-phenyl-5,6,7,8,9,10-hexahydropyrido[2,3-b][1,6]naphthyridine-4-carbonitrile (9B)

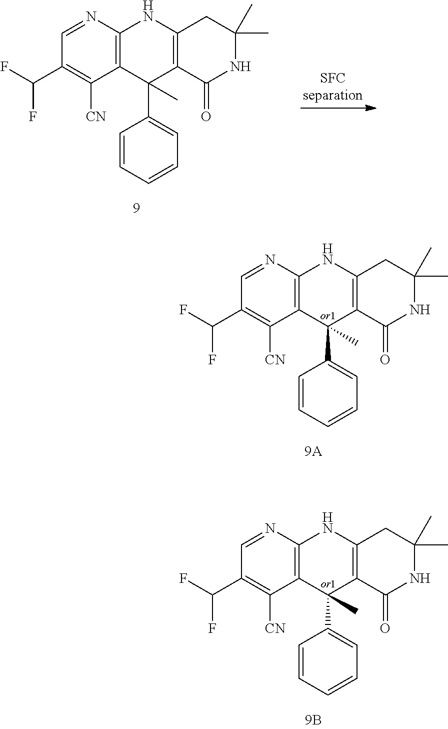 [SMILES: CC1(C)CC2=C(C(=O)N1)C(C)(c1ccccc1)c1c(ncc(C(F)F)c1C#N)N2 ; CC1(C)CC2=C(C(=O)N1)[C@@](C)(c1ccccc1)c1c(ncc(C(F)F)c1C#N)N2 ; CC1(C)CC2=C(C(=O)N1)[C@](C)(c1ccccc1)c1c(ncc(C(F)F)c1C#N)N2]

Chiral separation of 3-(difluoromethyl)-5,8,8-trimethyl-6-oxo-5-phenyl-5,6,7,8,9,10-hexahydropyrido[2,3-b][1,6]naphthyridine-4-carbonitrile (9) (20 mg, 0.05 mmol) was done by prep SFC—(Column: (R, R) WHELK-O1 (21.1 mm×250 mm), 5μ Flow: 60 mL/min Mobile Phase: 75% CO₂+25% (MeOH) ABPR: 100 bars; Temp: 35° C.; UV: 331 nm; diluent: Methanol, DCM & CH3CN). SFC fractions were collected and evaporated under reduced pressure to afford:

Peak 1: (S)-3-(difluoromethyl)-5,8,8-trimethyl-6-oxo-5-phenyl-5,6,7,8,9,10-hexahydropyrido [2,3-b][1,6]naphthyridine-4-carbonitrile (9A) (3.4 mg, 0.008 mmol, 99.20% purity, 17% yield).

UPLC: Rt=2.10 min., (ES⁺) m/z (M+H)⁺=395.2

¹H NMR (400 MHz, DMSO-de) δ=10.19 (bs, 1H), 8.37 (s, 1H), 7.34 (d, J=8.0 Hz, 2H), 7.22 (t, J=8.0 Hz, 2H), 7.12-6.85 (m, 2H), 6.57 (s, 1H), 2.38 (s, 2H) 2.24 (s, 3H), 1.16 (s, 3H), 1.08 (s, 3H).

[α]²⁵=+207.532, c=0.1783, MeOH.

Chiral purity (ee %): 100

Peak 2: (R)-3-(difluoromethyl)-5,8,8-trimethyl-6-oxo-5-phenyl-5,6,7,8,9,10-hexahydropyrido [2,3-b][1,6]naphthyridine-4-carbonitrile (9B) (3.54 mg, 0.01 mmol, 99.80% purity, 18% yield).

UPLC: Rt=2.10 min., (ES⁺) m/z (M+H)⁺=395.2

¹H NMR (400 MHz, DMSO-d₆) δ=10.19 (bs, 1H), 8.37 (s, 1H), 7.34 (d, J=8.0 Hz, 2H), 7.22 (t, J=8.0 Hz, 2H), 7.12-6.85 (m, 2H), 6.57 (s, 1H), 2.38 (s, 2H) 2.24 (s, 3H), 1.16 (s, 3H), 1.08 (s, 3H).

[α]²⁵=−217.244, c=0.1703, MeOH.

Chiral purity (ee %): 100

### Example 3.4. (S)-3-(difluoromethyl)-5,7,8,8-tetramethyl-6-oxo-5-phenyl-5,6,7,8,9,10-hexahydropyrido [2,3-b][1,6]naphthyridine-4-carbonitrile (9A) and (R)-3-(difluoromethyl)-5,7,8,8-tetramethyl-6-oxo-5-phenyl-5,6,7,8,9,10-hexahydropyrido[2,3-b][1,6]naphthyridine-4-carbonitrile (9B)

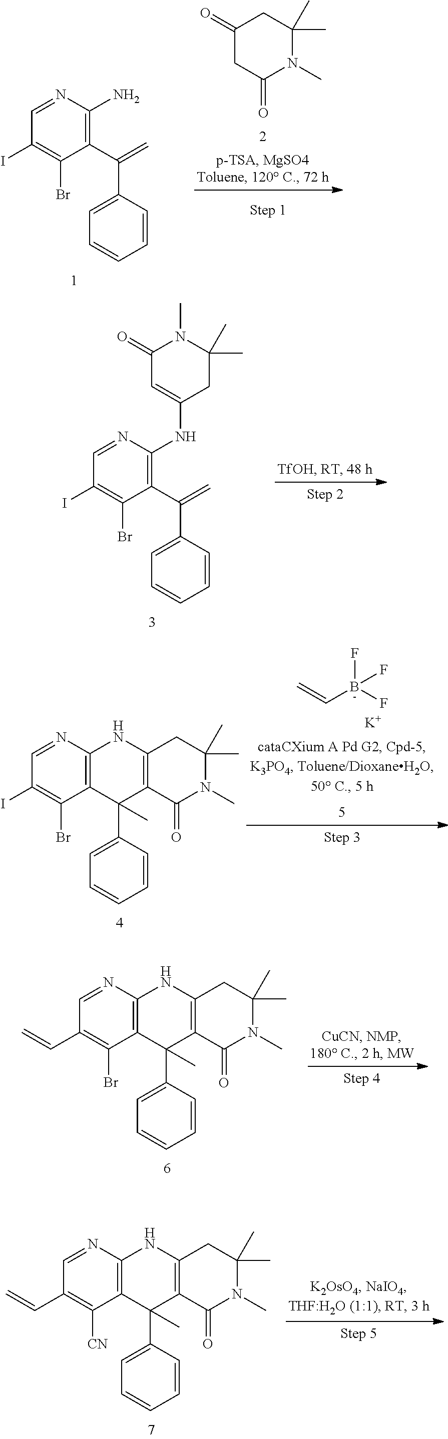

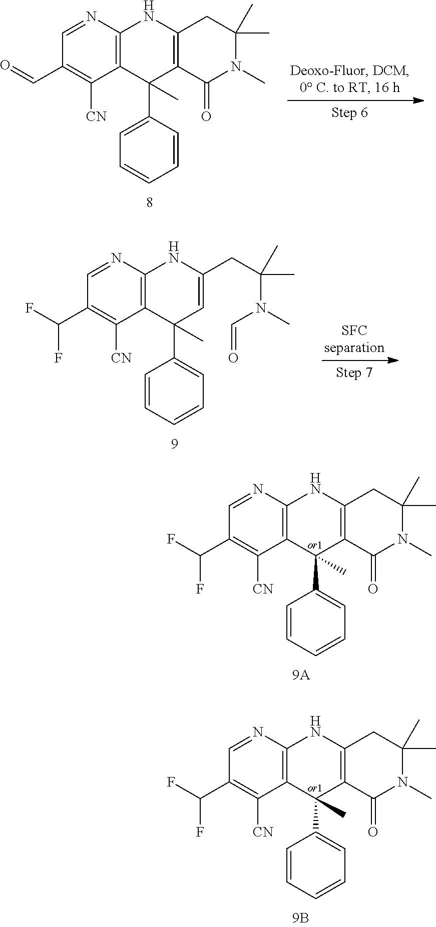 [SMILES: CN1C(=O)C2=C(CC1(C)C)Nc1ncc(C=O)c(C#N)c1C2(C)c1ccccc1 ; CN(C=O)C(C)(C)CC1=CC(C)(c2ccccc2)c2c(ncc(C(F)F)c2C#N)N1 ; CN1C(=O)C2=C(CC1(C)C)Nc1ncc(C(F)F)c(C#N)c1[C@]2(C)c1ccccc1 ; CN1C(=O)C2=C(CC1(C)C)Nc1ncc(C(F)F)c(C#N)c1[C@@]2(C)c1ccccc1]

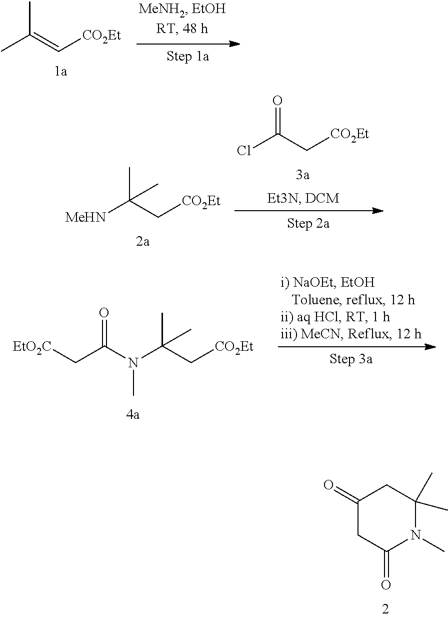

### Step 1: 4-((4-bromo-5-iodo-3-(I-phenylvinyl) pyridin-2-yl) amino)-1,6,6-trimethyl-5,6-dihydropyridin-2(1H)-one. (3)

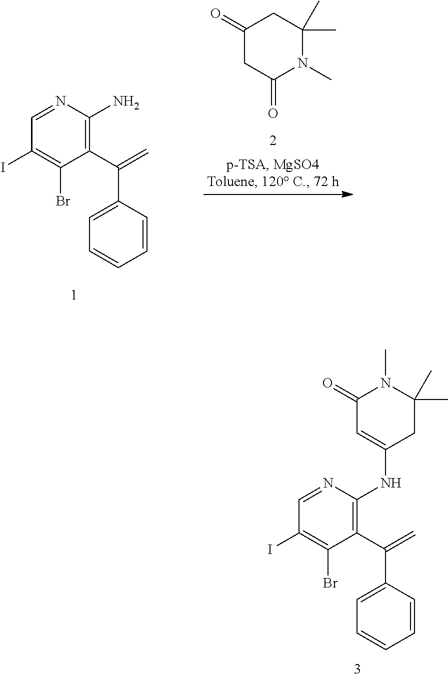 [SMILES: C=C(c1ccccc1)c1c(N)ncc(I)c1Br ; CN1C(=O)CC(=O)CC1(C)C ; C=C(c1ccccc1)c1c(NC2=CC(=O)N(C)C(C)(C)C2)ncc(I)c1Br]

To a stirred solution of 4-bromo-5-iodo-3-(l-phenylvinyl)pyridin-2-amine (2 g, 5 mmol, 1 eq) in toluene (80 mL) were sequentially added 1,6,6-trimethylpiperidine-2,4-dione (1.16 g, 7.48 mmol, 1.5 eq), p-Toluenesulfonic acid monohydrate (190 mg, 0.997 mmol, 0.2 eq) and MgSO₄ (2.39 g, 19.9 mmol, 4 eq). Reaction mixture was stirred at 130° C. for 72 h. On completion, reaction mixture was concentrated under reduced pressure to afford a brownish crude, which was purified by CombiFlash column chromatography using 2% MeOH in DCM to provide 4-[[4-bromo-5-iodo-3-(1-phenylvinyl)-2-pyridyl]amino]-1,2,2-trimethyl-3H-pyridin-6-one (3) (1.50 g, 2.79 mmol, 87.28% purity, 49% yield).

LCMS: (ES⁺) m/z (M+H)⁺=537.7, Rt=3.54 min

### Step 2: 4-bromo-3-iodo-5,7,8,8-tetramethyl-5-phenyl-5,8,9,10-tetrahydropyrido[2,3-b] [1,6] naphthyridin-6(7H)-one. (4)

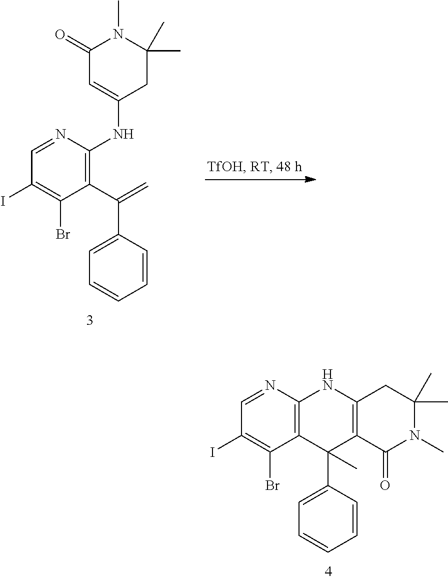 [SMILES: C=C(c1ccccc1)c1c(NC2=CC(=O)N(C)C(C)(C)C2)ncc(I)c1Br ; CN1C(=O)C2=C(CC1(C)C)Nc1ncc(I)c(Br)c1C2(C)c1ccccc1]

TfOH (10 mL) was dropwise added to RB flask containing 4-[[4-bromo-5-iodo-3-(1-phenylvinyl)-2-pyridyl] amino]-1,2,2-trimethyl-3H-pyridin-6-one (3) (1.20 g, 2.23 mmol, 1 eq) at 0° C. under inert atmosphere. Resulting mixture was allowed to stir at RT for 48 h. On completion, reaction mixture was poured slowly to the aqueous NaHCO₃ solution (1M) at 0° C. and extracted with EtOAc (3×100 mL). The combined organic layer was washed sequentially with aqueous NaHCO₃ (1M) solution, water and brine, dried over Na₂SO₄, filtered and concentrated under reduced pressure to get brownish crude. Obtained crude was purified by CombiFlash column chromatography using 50% EtOAc in hexane to provide 4-bromo-3-iodo-5,7,8,8-tetramethyl-5-phenyl-9,10-dihydropyrido[2,3-b][1,6]naphthyridin-6-one (4) (600 mg, 1.03 mmol, 92.35% purity, 46% yield).

LCMS: (ES⁺) m/z (M+H)⁺=537.9, Rt=2.05 min

### Step 3: 4-bromo-5,7,8,8-tetramethyl-5-phenyl-3-vinyl-5,8,9,10-tetrahydropyrido[2,3-b][1,6] naphthyridin-6(7H)-one. (6)

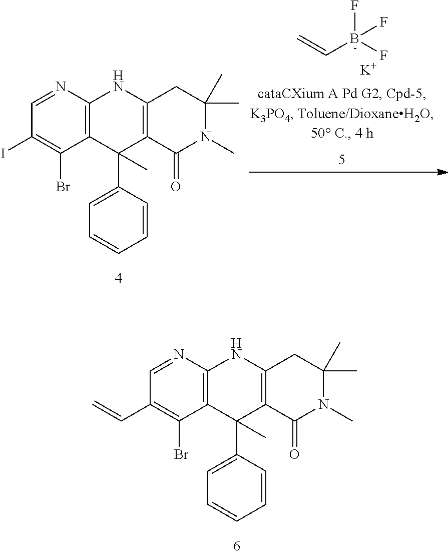

To a stirred solution of 4-bromo-3-iodo-5,7,8,8-tetramethyl-5-phenyl-9,10-dihydropyrido[2,3-b][1,6]naphthyridin-6-one (4) (600 mg, 1.11 mmol, 1.00 eq) in toluene-dioxane-water (4:4:1) (22 mL) were added potassium trifluoro(vinyl)boranuide (5) (209 mg, 1.56 mmol, 1.40 eq) and K₃PO₄ (473 mg, 2.23 mmol, 2.00 eq). Reaction mixture was purged with argon for 10 min then cataCXium A Pd G2 (37 mg, 0.06 mmol, 0.05 eq) was added and stirred at 50° C. for 4 h. Reaction mixture was diluted with water (5 mL) and extracted with EtOAc (3×50 mL). Combined organic layer was washed with water and brine, dried over Na₂SO₄, filtered and concentrated under reduced pressure to get a brownish crude. Obtained crude was purified with CombiFlash column chromatography using 30% EtOAc in hexane to provide 4-bromo-5,7,8,8-tetramethyl-5-phenyl-3-vinyl-9,10-dihydropyrido[2,3-b][1,6]naphthyridin-6-one (6) (200 mg, 0.411 mmol, 90% purity, 37% yield).

LCMS: (ES⁺) m/z (M+H)⁺=438.0, Rt=3.56 min

### Step 4: 5,7,8,8-tetramethyl-6-oxo-5-phenyl-3-vinyl-5,6,7,8,9,10-hexahydropyrido[2,3-b][1,6] naphthyridine-4-carbonitrile. (7)

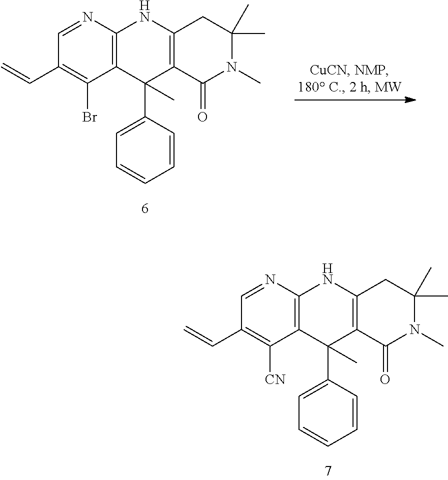 [SMILES: C=Cc1cnc2c(c1Br)C(C)(c1ccccc1)C1=C(CC(C)(C)N(C)C1=O)N2 ; C=Cc1cnc2c(c1C#N)C(C)(c1ccccc1)C1=C(CC(C)(C)N(C)C1=O)N2]

To a stirred solution of 4-bromo-5,7,8,8-tetramethyl-5-phenyl-3-vinyl-9,10-dihydropyrido[2,3-b] [1,6]naphthyridin-6-one (6) (190 mg, 0.433 mmol, 1 eq) in NMP (3 mL) taken in a microwave vial was added CuCN (194 mg, 2.17 mmol, 5 eq) and the reaction mixture was allowed to stir at 180° C. for 2 h under microwave irradiation. On completion, the reaction mixture was diluted water (4 mL) and extracted with EtOAc (2×25 mL). Combined organic layer was washed with brine, dried over Na₂SO₄, filtered and concentrated under reduced pressure to afford a yellowish crude. Crude was purified by CombiFlash column chromatography using 50% EtOAc-DCM to afford 5,7,8,8-tetramethyl-6-oxo-5-phenyl-3-vinyl-9,10-dihydropyrido[2,3-b][1,6]naphthyridine-4-carbonitrile (7) (70 mg, 0.175 mmol, 96.06% purity, 40% yield) as light yellow solid.

LCMS: (ES⁺) m/z (M+H)⁺=385.2, Rt=2.18 min

### Step 5: 3-formyl-5,7-dimethyl-6-oxo-5-phenyl-5,6,7,8,9,10-hexahydropyrido[2,3-b][1,6] naphthyridine-4-carbonitrile. (8)

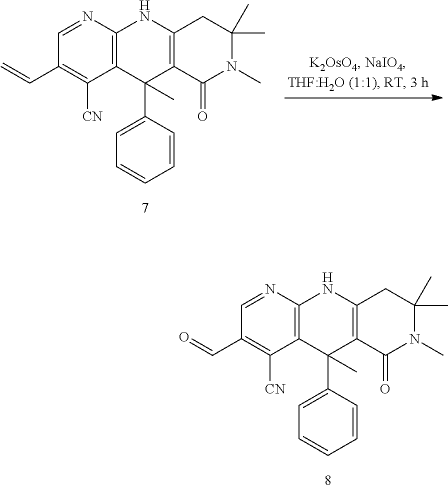 [SMILES: C=Cc1cnc2c(c1C#N)C(C)(c1ccccc1)C1=C(CC(C)(C)N(C)C1=O)N2 ; CN1C(=O)C2=C(CC1(C)C)Nc1ncc(C=O)c(C#N)c1C2(C)c1ccccc1]

To a stirred solution of 5,7,8,8-tetramethyl-6-oxo-5-phenyl-3-vinyl-9,10-dihydropyrido[2,3-b][1,6]naphthyridine-4-carbonitrile (7) (60 mg, 0.156 mmol, 1 eq) in THF:H₂O (1:1) (5 mL) were added Potassium osmate (2.6 mg, 0.008 mmol, 0.05 eq) and sodium periodate (167 mg, 0.78 mmol, 5 eq). Reaction mixture was stirred at RT for 3 h. On completion, the reaction mixture was diluted with water (2 mL) and extracted with EtOAc (2×20 mL). Combined organic layer was washed with brine, dried over Na₂SO₄, filtered and concentrated under reduced pressure to provide crude 3-formyl-5,7,8,8-tetramethyl-6-oxo-5-phenyl-9,10-dihydropyrido[2,3-b][1,6] naphthyridine-4-carbonitrile (8) (50 mg, 0.116 mmol, 89.79% purity, 74% yield). Obtained crude was used for the next step without further purification.

LCMS: (ES⁺) m/z (M+H)⁺=387.2, Rt=1.68 min

### Step 6: 3-(difluoromethyl)-5,7,8,8-tetramethyl-6-oxo-5-phenyl-5,6,7,8,9,10-hexahydropyrido [2,3-b][1,6]naphthyridine-4-carbonitrile. (9)

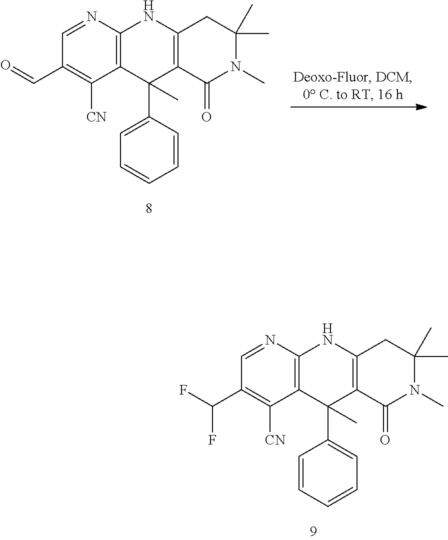 [SMILES: CN1C(=O)C2=C(CC1(C)C)Nc1ncc(C=O)c(C#N)c1C2(C)c1ccccc1 ; CN1C(=O)C2=C(CC1(C)C)Nc1ncc(C(F)F)c(C#N)c1C2(C)c1ccccc1]

To a stirred solution of 3-formyl-5,7,8,8-tetramethyl-6-oxo-5-phenyl-9,10-dihydropyrido[2,3-b] [1,6]naphthyridine-4-carbonitrile (8) (55 mg, 0.142 mmol, 1 eq) in DCM (1.5 mL) was added Deoxo-Fluor (0.39 mL, 2.13 mmol, 15.0 eq) at 0° C. under inert atmosphere. Reaction mixture was allowed to stir at RT for 16 h. On completion, reaction mixture was diluted with aqueous NaHCO₃ solution (2 mL) and extract with DCM (2×20 mL). Combined organic layer was dried over Na₂SO₄, filtered and concentrated under reduced pressure to get a yellowish gummy crude. Obtained crude was purified by prep TLC using 30% EtOAc in hexane to provide 3-(difluoromethyl)-5,7,8,8-tetramethyl-6-oxo-5-phenyl-9,10-dihydropyrido[2,3-b][1,6] naphthyridine-4-carbonitrile (9) (30 mg, 0.071 mmol, 96.77% purity, 50% yield) as yellow solid.

LCMS: (ES⁺) m/z (M+H)⁺=409.4, Rt=2.14 min

### Step 7: Chiral Separation

### (S)-3-(difluoromethyl)-5,7,8,8-tetramethyl-6-oxo-5-phenyl-5,6,7,8,9,10-hexahydropyrido [2,3-b][1,6]naphthyridine-4-carbonitrile (9A) and (R)-3-(difluoromethyl)-5,7,8,8-tetramethyl-6-oxo-5-phenyl-5,6,7,8,9,10-hexahydropyrido[2,3-b][1,6] naphthyridine-4-carbonitrile (9B)

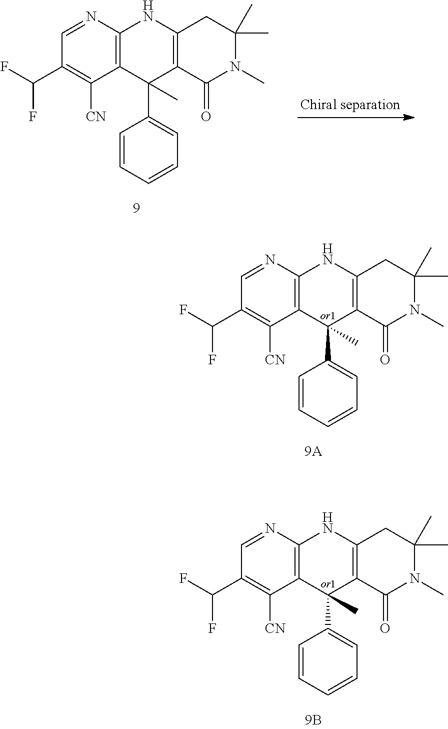 [SMILES: CN1C(=O)C2=C(CC1(C)C)Nc1ncc(C(F)F)c(C#N)c1C2(C)c1ccccc1 ; CN1C(=O)C2=C(CC1(C)C)Nc1ncc(C(F)F)c(C#N)c1[C@]2(C)c1ccccc1 ; CN1C(=O)C2=C(CC1(C)C)Nc1ncc(C(F)F)c(C#N)c1[C@@]2(C)c1ccccc1]

Chiral separation of 3-(difluoromethyl)-5,7,8,8-tetramethyl-6-oxo-5-phenyl-9,10-dihydropyrido[2,3-b][1,6] naphthyridine-4-carbonitrile (9) (30 mg, 0.071 mmol) was done by prep SFC (Column: Reflect(R,R)Whelk_O1 (21.1 mm×250 mm), 5μ Flow: 60 mL/min Mobile Phase: 65% CO₂+35% (MeOH), ABPR: 100 bars; Temp: 35° C.; UV: 220 nm; diluent: Methanol & DCM.

SFC fractions were collected and evaporated under reduced pressure to afford:

Peak 1: (S)-3-(difluoromethyl)-5,7,8,8-tetramethyl-6-oxo-5-phenyl-5,6,7,8,9,10-hexahydropyrido [2,3-b][1,6]naphthyridine-4-carbonitrile (9A) (13.79 mg, 0.03 mmol, 98.10% purity, 34% yield) as yellow solid.

UPLC: Rt=2.25 min., (ES⁺) m/z (M+H)⁺=409.2

¹H NMR (400 MHz, DMSO-d₆) δ=10.20 (s, 1H), 8.38 (s, 1H), 7.34 (d, J=8.0 Hz, 2H), 7.21 (t, J=8.0 Hz, 2H), 7.10-7.07 (m, 1H), 6.99 (t, J=54 Hz, 1H), 2.55 (s, 3H), 2.50-2.46 (m, 2H, merged with DMSO peak), 2.22 (s, 3H) 1.23 (s, 3H), 1.10 (s, 3H).

[α]²⁵=+247.485, c=0.1814, MeOH.

Chiral purity (ee %): 100

Peak 2: (R)-3-(difluoromethyl)-5,7,8,8-tetramethyl-6-oxo-5-phenyl-5,6,7,8,9,10-hexahydro pyrido[2,3-b][1,6]naphthyridine-4-carbonitrile (9B) (15.19 mg, 0.04 mmol, 98.61% purity, 37% yield) as yellow solid.

LCMS: Rt=2.25 min., (ES⁺) m/z (M+H)⁺=409.2

¹H NMR (400 MHz, DMSO-de) δ=10.20 (s, 1H), 8.38 (s, 1H), 7.34 (d, J=8.0 Hz, 2H), 7.21 (t, J=8.0 Hz, 2H), 7.10-7.07 (m, 1H), 6.99 (t, J=54 Hz, 1H), 2.55 (s, 3H), 2.50-2.46 (m, 2H, merged with DMSO peak), 2.22 (s, 3H) 1.23 (s, 3H), 1.10 (s, 3H).

[α]²⁵=−246.053, c=0.1650, MeOH.

Chiral purity (ee %): 100

### Step 1a: ethyl 3-methyl-3-(methylamino)butanoate (2a)

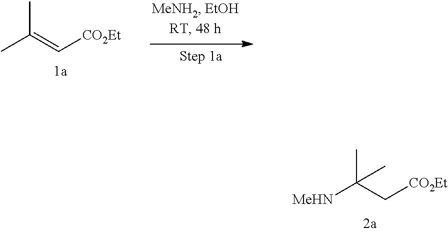

To a solution of ethyl 3-methylbut-2-enoate (5.00 g, 39.0 mmol, 1.00 eq) in Methyl amine in ethanol (30 mL) was stirred at RT for 48 h. Reaction mixture was concentrated under reduced pressure to provide ethyl 3-methyl-3-(methylamino)butanoate, (5.10 g, 32.0 mmol, 100%, 82% yield) as colorless oil.

¹H NMR (400 MHz, DMSO-de) δ 4.13-4.08 (m, 2H), 2.30 (s, 3H), 2.13 (s, 1H), 1.86 (s, 1H), 1.28-1.20 (m, 3H), 1.13 (s, 6H).

### Step 2a: ethyl 3-(3-ethoxy-N-methyl-3-oxopropanamido)-3-methylbutanoate (4a)

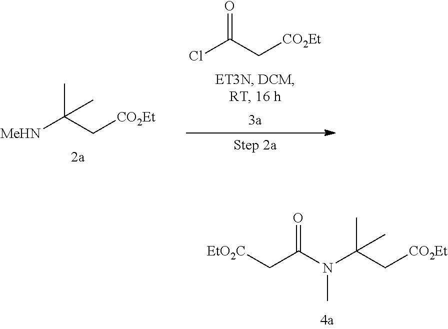 [SMILES: CCOC(=O)CC(=O)Cl ; CCOC(=O)CC(C)(C)NC ; CCOC(=O)CC(=O)N(C)C(C)(C)CC(=O)OCC]

To a stirred solution of ethyl 3-methyl-3-(methylamino)butanoate (5.20 g, 32.7 mmol, 1.00 eq) in DCM (30 mL) were added Et3N (6.83 mL, 49.0 mmol, 1.50 eq) ethyl 3-chloro-3-oxo-propanoate (5.04 mL, 39.2 mmol, 1.20 eq) at 0° C. under inert atmosphere. Reaction mixture was stirred at RT for 16 h. Reaction mixture was quenched with cold water (5 mL) and extracted with DCM (2×300 mL). Combined organic part was collected, washed with brine, dried over Na2SO4, filtered and concentrated under reduced pressure to get crude product which was purified by combi flash chromatography (Column: SiO2, 40 g) using 20%-30% EtOAc in hexane to provide ethyl 3-[(3-ethoxy-3-oxo-propanoyl)-methyl-amino]-3-methyl-butanoate (2.90 g, 10.6 mmol, ?, 32% yield) as colorless sticky gum.

¹H NMR (400 MHz, DMSO-d₆) δ 4.13-3.99 (m, 4H), 3.44 (s, 2H), 2.95 (s, 2H), 2.84 (s, 3H), 1.39 (s, 6H), 1.19-1.14 (m, 6H).

### Step 3a: 1,6,6-trimethylpiperidine-2,4-dione (2)

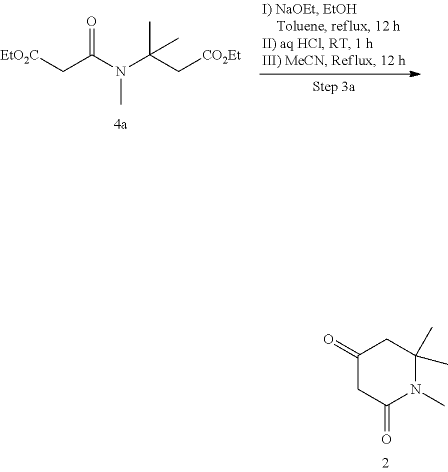 [SMILES: CCOC(=O)CC(=O)N(C)C(C)(C)CC(=O)OCC ; CN1C(=O)CC(=O)CC1(C)C]

Methyl 3-[(3-ethoxy-3-oxo-propanoyl)-methyl-amino]-3-methyl-butanoate (4a) (3 g, 11.6 mmol, eq) in toluene (20 mL) was slowly added to the mixture of Sodium Ethoxide in Ethanol (1.36 mL, 17.4 mmol, 1.50 eq) at 0° C. under inert atmosphere. Then the resulting mixture was heated at 110° C. for 12 h. After that the reaction mixture was cooled to and the aqueous phase made acidic with 2N HCL. Then the mixture was extracted with EtOAc. The organic phase was separated, dried over sodium sulfate and concentrated under reduced pressure. The residue was mixed with ACN containing 1% water and refluxed for 18 h. After completion, the reaction mass was concentrated under reduced pressure to afford the crude material. Crude was purified by combi-flash column chromatography using silica-gel column and 70%-80% ethyl acetate in hexane to elute the compound 1,6,6-trimethylpiperidine-2,4-dione, (35 mg, 0.226 mmol, 58% yield) as brown solid.

LCMS: (ES⁺) m/z (M+H)⁺=156.2, tR=1.12 min

### Example 3.5. (S)-3-fluoro-5,8,5-trimethyl-6-oxo-5-phenyl-5,6,7,8,9,10-hexahydropyrido[2,3-b][1,6]naphthyridine-4-carbonitrile (9A) & (R)-3-fluoro-5,8,8-trimethyl-6-oxo-5-phenyl-5,6,7,8,9,10-hexahydropyrido[2,3-b][1,6]naphthyridine-4-carbonitrile (9B)

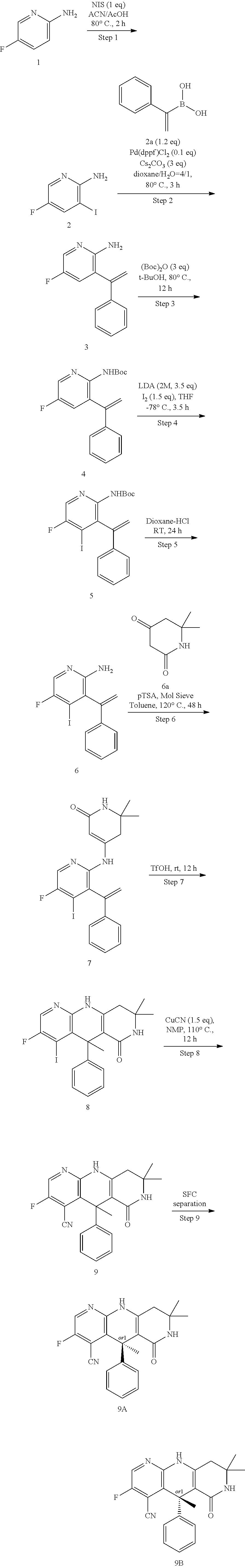

### Step 1: 5-fluoro-3-iodopyridin-2-amine (2)

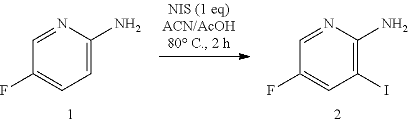 [SMILES: Nc1ccc(F)cn1 ; Nc1ncc(F)cc1I]

To a stirred solution of 5-fluoropyridin-2-amine (1) (5 g, 44.6 mmol, 1 eq) and N-Iodosuccinimide (10 g, 44.6 mmol, 1 eq) in acetonitrile (100 mL) was added acetic acid (0.26 ml, 0.1 eq) at 0° C. under inert atmosphere. Resulting mixture was allowed to stir at 80° C. for 2 h. On completion, reaction mixture was quenched with aqueous sodium thiosulfate solution (50 ml) and aqueous NaHCO₃ solution under stirring condition for 30 min. Obtained mixture was extracted with ethyl acetate (2×300 ml) and the combined organic layer was dried over Na₂SO₄, filtered and concentrated to obtain a yellowish crude. Crude was purified by CombiFlash chromatography using 10-15% ethyl acetate in hexane gradient to furnish 5-fluoro-3-iodo-pyridin-2-amine (2) (2.60 g, 10.9 mmol, 100% purity, 24% yield) as off white solid.

¹H NMR (400 MHz, DMSO-d6) 7.96-7.93 (m, 2H), 5.98 (s, 2 h).

### Step 2: 5-fluoro-3-(1-phenylvinyl) pyridin-2-amine (3)

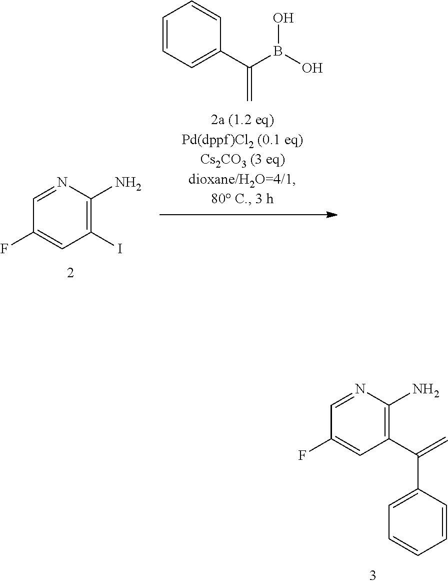 [SMILES: C=C(B(O)O)c1ccccc1 ; Nc1ncc(F)cc1I ; C=C(c1ccccc1)c1cc(F)cnc1N]

To the degassed solution of 5-fluoro-3-iodo-pyridin-2-amine (2) (1 g, 4.20 mmol, 1 eq), 1-phenylvinylboronic acid (2a) (0.75 g, 5.04 mmol, 1.20 eq) and cesium carbonate (4.11 g, 12.6 mmol, 3 eq) in 4:1 dioxane-water mixture (20 mL) argon was purged for 15 mins. Pd(dppf)Cl₂ DCM (0.34 g, 0.420 mmol, 0.1 eq) was then added and allowed to stir at 80° C. for 3 h. Reaction mixture was diluted with water and extracted with ethyl acetate (2×100 ml). Combined organic layer was collected, washed with brine, dried over Na₂SO₄, filtered off and concentrated to get a brownish crude. Crude was purified by CombiFlash chromatography using 20-30% ethyl acetate in hexane gradient to afford 5-fluoro-3-(1-phenylvinyl) pyridin-2-amine (3) (600 mg, 2.76 mmol, 98.66% purity, 66% yield) as solid.

LCMS: Rt=1.85 min., (ES⁺) m/z (M+H)⁺=215.2

### Step 3: tert-butyl (5-fluoro-3-(1-phenylvinyl) pyridin-2-yl) carbamate (4)

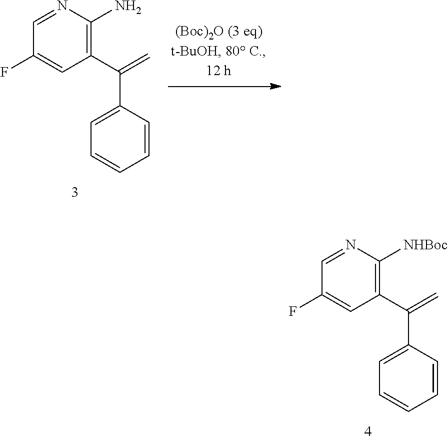 [SMILES: C=C(c1ccccc1)c1cc(F)cnc1N ; C=C(c1ccccc1)c1cc(F)cnc1NC(=O)OC(C)(C)C]

To the solution of 5-fluoro-3-(1-phenylvinyl) pyridin-2-amine (3) (600 mg, 2.80 mmol, 1 eq) in tert-butanol (5 mL) was added Boc₂O (1.93 mL, 8.40 mmol, 3 eq) at RT and the resulting mixture was allowed to stir at 80° C. for 12 h. On completion, volatiles were removed under reduced pressure to get a colourless crude. Obtained crude was purified by CombiFlash chromatography using 2-5% Ethyl acetate in hexane gradient to afford tert-butyl N-[5-fluoro-3-(1-phenylvinyl)-2-pyridyl]carbamate (4) (500 mg, 1.51 mmol, 95% purity, 54% yield) as brownish solid.

LCMS: Rt=3.57 min., (ES⁺) m/z (M+H)⁺=315.5

### Step 4: tert-butyl (5-fluoro-4-iodo-3-(1-phenylvinyl) pyridin-2-yl) carbamate (5)

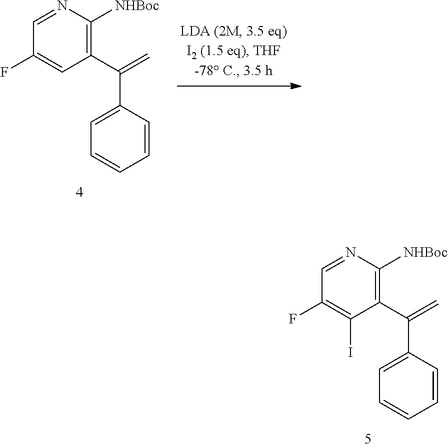 [SMILES: C=C(c1ccccc1)c1cc(F)cnc1NC(=O)OC(C)(C)C ; C=C(c1ccccc1)c1c(NC(=O)OC(C)(C)C)ncc(F)c1I]

To the stirred solution of tert-butyl N-[5-fluoro-3-(1-phenylvinyl)-2-pyridyl]carbamate (4) (6.50 g, 20.7 mmol, 1 eq) in THF (100 mL), 2M LDA in THF (36.2 mL, 72.4 mmol, 3.5 eq) was added dropwise at −78° C. under inert atmosphere and allowed to stir at −78° C. for 1 h. Iodine (7.88 g, 31.0 mmol, 1.5 eq) dissolved in THF (30 mL) was added dropwise under same temperature and stirred for 3.5 h. Reaction mixture was quenched with aqueous ammonium chloride solution (100 ml), aqueous sodium thiosulfate solution (200 ml) and was extracted with ethyl acetate (4×150 ml). Combined organic layer was dried over Na₂SO₄, filtered and concentrated to afford a brownish crude. Crude was purified by CombiFlash chromatography using 2-4% Ethyl acetate in hexane gradient to obtained tert-butyl N-[5-fluoro-4-iodo-3-(1-phenylvinyl)-2-pyridyl]carbamate (5) (3.20 g, 7.11 mmol, 97.76% purity, 34% yield) as brownish solid.

LCMS: Rt=2.20 min., (ES⁺) m/z (M+H)⁺=441.2

### Step 5: 5-fluoro-4-iodo-3-(1-phenylvinyl) pyridin-2-amine (6)

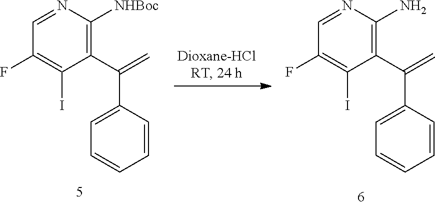 [SMILES: C=C(c1ccccc1)c1c(NC(=O)OC(C)(C)C)ncc(F)c1I ; C=C(c1ccccc1)c1c(N)ncc(F)c1I]

To the solution of tert-butyl N-[5-fluoro-4-iodo-3-(1-phenylvinyl)-2-pyridyl] carbamate (5) (500 mg, 1.14 mmol, 1 eq) in 1,4 Dioxane (3 mL) was dropwise added 4 M HCl in 1,4 Dioxane (5 mL) at 0° C. under inert atmosphere. Resulting mixture was allowed to stir at RT for 24 h. On completion, reaction mixture was quenched with aqueous NaHCO₃ solution and extracted with ethyl acetate (2×100 ml). Combine organic part was washed with brine, dried over Na₂SO₄, filtered and concentrated under reduced pressure to afford 5-fluoro-4-iodo-3-(1-phenylvinyl) pyridin-2-amine (6) (280 mg, 0.741 mmol, 90% purity, 65% yield) as brownish solid.

LCMS: Rt=3.40 min., (ES⁺) m/z (M+H)⁺=340.8.

### Step 6: 4-((5-fluoro-4-iodo-3-(I-phenylvinyl) pyridin-2-yl) amino)-6,6-dimethyl-5,6-dihydropyridin-2(1H)-one (7)

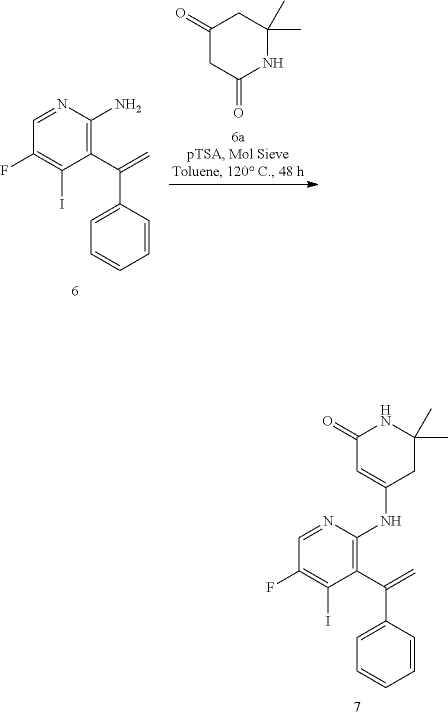

To a stirred solution of 5-fluoro-4-iodo-3-(I-phenylvinyl) pyridin-2-amine (6) (900 mg, 2.65 mmol, 1 eq) in toluene (20 mL) were sequentially added 6,6-dimethylpiperidine-2,4-dione (6a) (448 mg, 3.18 mmol, 1.2 eq), PTSA (101 mg, 0.529 mmol, 0.2 eq) and MS 4A (250 mg) under inert atmosphere. Resulting mixture was allowed to stir at 120° C. for 48 h. On completion, reaction mixture was filtered through celite, washed with EtOAc and the obtained filtrate was evaporated under reduced pressure to afford a brownish crude. Obtained crude was purified by CombiFlash column chromatography using 20-60% EtOAc in hexane gradient to afford 4-[[5-fluoro-4-iodo-3-(1-phenylvinyl)-2-pyridyl]amino]-2,2-dimethyl-1,3-dihydropyridin-6-one (7) (420 mg, 0.907 mmol, 94% purity, 34% yield) as brownish solid.

LCMS: Rt=3.27, (ES⁺) m/z (M+H)⁺=463.8 [M+H]

### Step 7: 3-fluoro-4-iodo-5,8,8-trimethyl-5-phenyl-5,8,9,10-tetrahydropyrido[2,3-b][1,6]naphthyridin-6(7H)-one (8)

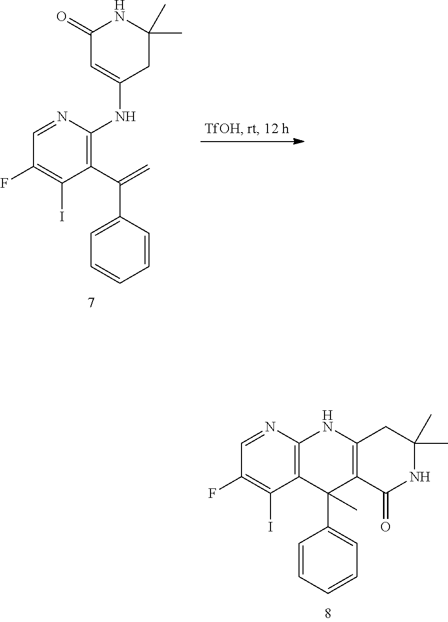 [SMILES: C=C(c1ccccc1)c1c(NC2=CC(=O)NC(C)(C)C2)ncc(F)c1I ; CC1(C)CC2=C(C(=O)N1)C(C)(c1ccccc1)c1c(ncc(F)c1I)N2]

Triflic acid (5 mL) was dropwise added to an RB flask, containing 4-[[5-fluoro-4-iodo-3-(1-phenylvinyl)-2-pyridyl]amino]-2,2-dimethyl-1,3-dihydropyridin-6-one (7) (410 mg, 0.885 mmol, 1.00 eq) at 0° C. under inert atmosphere. Resulting mixture was allowed to stir at RT for 12 h. On completion, reaction mixture was quenched with sat. NaHCO₃ solution and extracted with EtOAc (2×100 ml). Combined organic layer was washed with brine, dried over Na₂SO₄, concentrated under reduced pressure to afford a brownish crude, which was triturated by diethylether to afford 3-fluoro-4-iodo-5,8,8-trimethyl-5-phenyl-9,10-dihydro-7H-pyrido[2,3-b][1,6]naphthyridin-6-one (8) (320 mg, 0.691 mmol, 87% purity, 78% yield) as off white solid.

LCMS: Rt=3.34, (ES⁺) m/z (M+H)⁺=463.8 [M+H]

### Step 8: 3-fluoro-5,8,8-trimethyl-6-oxo-5-phenyl-5,6,7,8,9,10-hexahydropyrido[2,3-b][1,6]naphthyridine-4-carbonitrile (9)

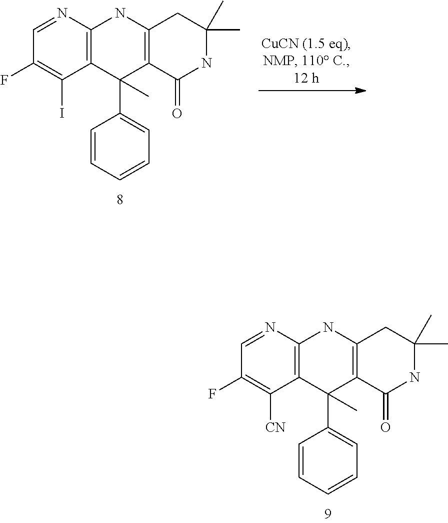 [SMILES: CC1(C)CC2=C(C(=O)N1)C(C)(c1ccccc1)c1c(ncc(F)c1I)N2 ; CC1(C)CC2=C(C(=O)N1)C(C)(c1ccccc1)c1c(ncc(F)c1C#N)N2]

To a stirred solution of 3-fluoro-4-iodo-5,8,8-trimethyl-5-phenyl-9,10-dihydro-7H-pyrido[2,3-b][1,6]naphthyridin-6-one (8) (310 mg, 0.669 mmol, 1 eq) in NMP (7 mL), taken in a sealed tube, was added CuCN (90 mg, 1.00 mmol, 1.5 eq) and the resulting mixture was allowed to stir at 110° C. for 12 h. On completion, reaction mixture was diluted with cold water and extracted with EtOAc (twice). Combined organic layer was washed with cold water and brine, and dried over Na₂SO₄, filtered and concentrated under reduced pressure to afford a yellowish crude. Obtained crude was purified by CombiFlash column chromatography to afford 3-fluoro-5,8,8-trimethyl-6-oxo-5-phenyl-5,6,7,8,9,10-hexahydropyrido[2,3-b][1,6]naphthyridine-4-carbonitrile (9) (140 mg, 0.386 mmol, 87% purity, 58% yield) as off white solid.

LCMS (m/z): Rt=1.74, (ES⁺) m/z (M+H)⁺=363.8

### Step 9: Chiral Separation

### (S)-3-fluoro-5,8,8-trimethyl-6-oxo-5-phenyl-5,6,7,8,9,10-hexahydropyrido[2,3-b][1,6]naphthyridine-4-carbonitrile (9A) & (R)-3-fluoro-5,8,8-trimethyl-6-oxo-5-phenyl-5,6,7,8,9,10-hexahydropyrido[2,3-b][1,6]naphthyridine-4-carbonitrile (9B)

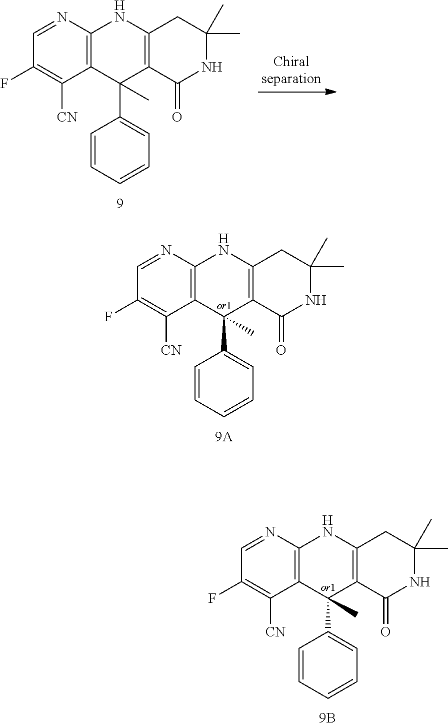 [SMILES: CC1(C)CC2=C(C(=O)N1)C(C)(c1ccccc1)c1c(ncc(F)c1C#N)N2 ; CC1(C)CC2=C(C(=O)N1)[C@@](C)(c1ccccc1)c1c(ncc(F)c1C#N)N2 ; CC1(C)CC2=C(C(=O)N1)[C@](C)(c1ccccc1)c1c(ncc(F)c1C#N)N2]

Chiral separation of 3-fluoro-5,8,8-trimethyl-6-oxo-5-phenyl-9,10-dihydro-7H-pyrido[2,3-b][1,6]naphthyridine-4-carbonitrile (9) (130 mg, 0.386 mmol) was done by prep SFC on Agilent series instrument. Column name: CHIRALPAK IG (250×30 mm) 5μ. Operating at ambient temperature and flow rate is 27.0 mL/min. Mobile phase was mixture of 60% Hexane, 20% Ethyl acetate, 20% EtOH and 0.1% Diethyl amine, held this isocratic mixture run up to 18 min with wavelength of 324 nm.

SFC fractions were collected and evaporated under reduced pressure to afford:

Peak 1: (S)-3-fluoro-5,8,8-trimethyl-6-oxo-5-phenyl-5,6,7,8,9,10-hexahydropyrido[2,3-b][1,6]naphthyridine-4-carbonitrile (9A) (16 mg, 0.0431 mmol, 97.42% purity, 12% yield).

UPLC: Rt=2.14, (ES⁺) m/z (M+H)⁺=363.3

¹H NMR (400 MHz, DMSO-d6) 9.88 (s, 1H), 8.34 (s, 1H), 7.35 (d, J=7.6 Hz, 2H), 7.22 (t, J=7.6 Hz, 2H), 7.09 (t, J=7.2 Hz, 1H), 6.53 (s, 1H), 2.37-2.33 (m, 1H), 2.21 (s, 3H), 1.16 (s, 3H), 1.08 (s, 3H).

[α]²⁵=+224.499, c=0.1265, DMSO.

Chiral purity (ee %): 97.1

Peak 2: (R)-3-fluoro-5,8,8-trimethyl-6-oxo-5-phenyl-5,6,7,8,9,10-hexahydropyrido[2,3-b][1,6]naphthyridine-4-carbonitrile (9B) (16 mg, 0.0452 mmol, 99.6% purity, 13% yield).

UPLC: Rt=2.12, (ES⁺) m/z (M+H)⁺=363.3

¹H NMR (400 MHz, DMSO-d6) 9.88 (s, 1H), 8.34 (s, 1H), 7.35 (d, J=7.6 Hz, 2H), 7.22 (t, J=7.6 Hz, 2H), 7.09 (t, J=7.2 Hz, 1H), 6.53 (s, 1H), 2.37-2.33 (m, 1H), 2.21 (s, 3H), 1.16 (s, 3H), 1.08 (s, 3H).

[α]²⁵=−199.191, c=0.1245, DMSO.

Chiral purity (ee %): 99.66.

### Example 3.6. (S)-3-fluoro-5,7,8,8-tetramethyl-1-oxo-5-phenyl-5,6,7,8,9,10-hexahydropyido[2,3-b][1,6]naphthyridine-4-carbonitrile (5A) and (R)-3-fluoro-5,7,8,8-tetramethyl-6-oxo-5-phenyl-5,6,7,8,9,10-hexahydropyrido[2,3-b][1,6]naphthyridine-4-carbonitrile (5B)

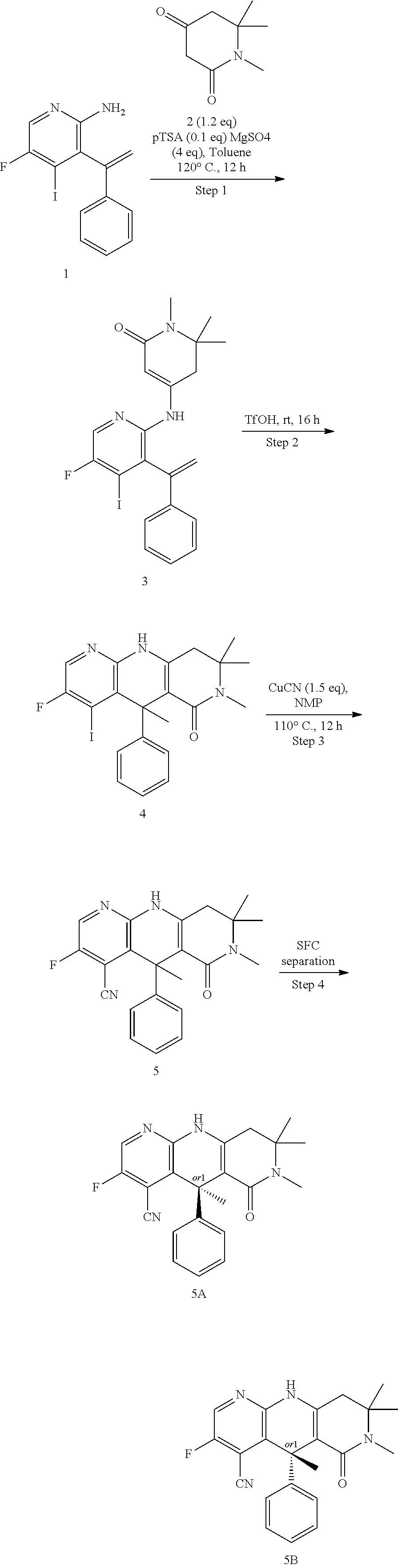

### Step 1: 4-((5-fluoro-4-iodo-3-(1-phenylvinyl)pyridin-2-yl)amino)-1,6,6-trimethyl-5,6-dihydropyridin-2(1H)-one (3)

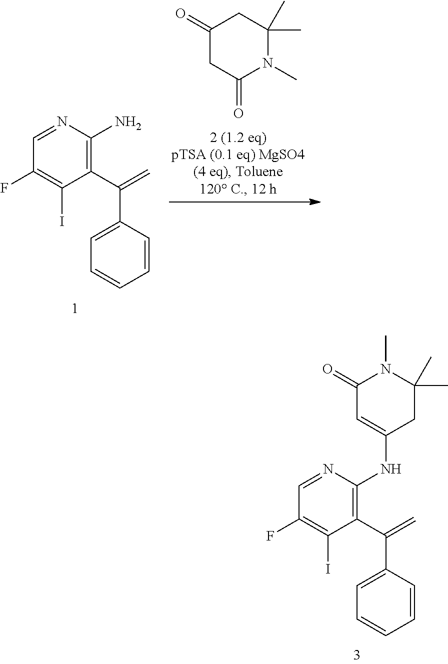 [SMILES: CN1C(=O)CC(=O)CC1(C)C ; C=C(c1ccccc1)c1c(N)ncc(F)c1I ; C=C(c1ccccc1)c1c(NC2=CC(=O)N(C)C(C)(C)C2)ncc(F)c1I]

To a stirred solution of 5-fluoro-4-iodo-3-(1-phenylvinyl) pyridin-2-amine (1) (450 mg, 1.32 mmol, 1 eq) in Toluene (15 mL) was added 1,6,6-trimethylpiperidine-2,4-dione (2) (246 mg, 1.59 mmol, 1.2 eq), MgSO4 (635 mg, 5.29 mmol, 4 eq) and pTSA (50 mg, 0.265 mmol, 0.1 eq) successively and then the reaction mass was stirred at 120° C. for 12 h. After completion of the reaction, the reaction mixture was filtered through celite pad and washed with EtOAc. The filtrate was then evaporated under vacuum to afford the crude. The crude thus obtained was purified by Combi-Flash column chromatography in 40-50% EtOAc in hexane to afford the desired product 4-[[5-fluoro-4-iodo-3-(1-phenylvinyl)-2-pyridyl]amino]-1,2,2-trimethyl-3H-pyridin-6-one (3) (350 mg, 0.733 mmol, 95% purity, 52% yield) as brownish solid.

LCMS: Rt=3.43 min., (ES⁺) m/z (M+H)⁺=478.0

### Step 2: 3-fluoro-4-iodo-5,7,8,8-tetramethyl-5-phenyl-5,8,9,10-tetrahydropyrido[2,3-b][1,6]naphthyridin-6(7H)-one (4)

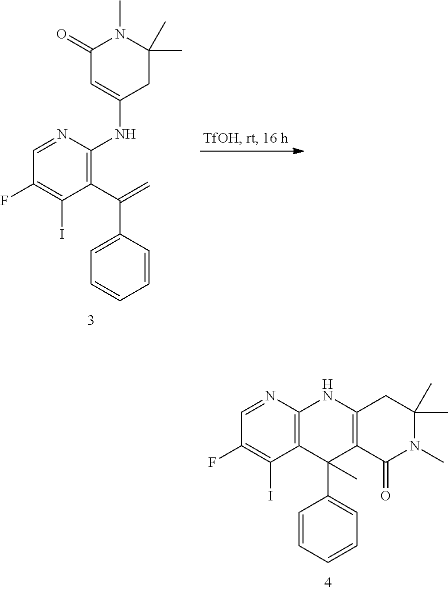 [SMILES: C=C(c1ccccc1)c1c(NC2=CC(=O)N(C)C(C)(C)C2)ncc(F)c1I ; CN1C(=O)C2=C(CC1(C)C)Nc1ncc(F)c(I)c1C2(C)c1ccccc1]

To 4-[[5-fluoro-4-iodo-3-(1-phenylvinyl)-2-pyridyl]amino]-1,2,2-trimethyl-3H-pyridin-6-one (350 mg, 0.733 mmol, 1 eq) was added Triflic acid (5 mL) at 0° C., then the reaction mass was stirred at 25° C. for 16 h. After completion of the reaction, the reaction mass was neutralized by sat. NaHCO3 solution and extracted with Ethyl Acetate. The organic layer was then washed with water, brine and dried with Na2SO4, concentrated under vacuum to afford the crude. The crude thus obtained was washed with diethyl ether to afford the 3-fluoro-4-iodo-5,7,8,8-tetramethyl-5-phenyl-5,8,9,10-tetrahydropyrido[2,3-bi 1,6]naphthyridin-6(7H)-one (4) which was used for the next step without any further purification.

LCMS: Rt=1.93 min., (ES⁺) m/z (M+H)=478.2

### Step 3: 3-fluoro-5,7,8,8-tetramethyl-6-oxo-5-phenyl-5,6,7,8,9,10-hexahydropyrido[2,3-b][1,6]naphthyridine-4-carbonitrile (5)

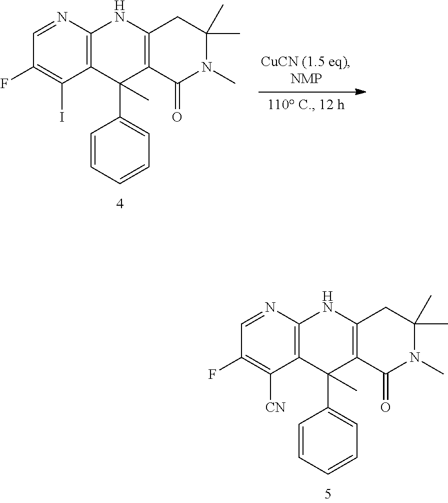 [SMILES: CN1C(=O)C2=C(CC1(C)C)Nc1ncc(F)c(I)c1C2(C)c1ccccc1 ; CN1C(=O)C2=C(CC1(C)C)Nc1ncc(F)c(C#N)c1C2(C)c1ccccc1]

To a stirred solution of 3-fluoro-4-iodo-5,7,8,8-tetramethyl-5-phenyl-5,8,9,10-tetrahydropyrido[2,3-b][1,6]naphthyridin-6(7H)-one (4) (160 mg, 0.335 mmol, 1 eq) in NMP (4 mL) was added CuCN (45 mg, 0.503 mmol, 1.5 eq) and stirred for 12 h at 110° C. in a sealed tube. On completion, chilled water was added to the reaction mixture and was extracted with EtOAc. The organic layer was then washed with water, brine and dried with Na2SO4, concentrated under vacuum to afford the crude. The crude was then purified by reverse phase column chromatography to afford 3-fluoro-5,7,8,8-tetramethyl-6-oxo-5-phenyl-5,6,7,8,9,10-hexahydropyrido[2,3-b][1,6]naphthyridine-4-carbonitrile (5) (55 mg, 0.146 mmol, 95% purity, 42% yield) as off white solid.

LCMS: Rt=3.18 min., (ES⁺) m/z (M+H)=377.2

### Step 4: (S)-3-fluoro-5,7,8,8-tetramethyl-6-oxo-5-phenyl-5,6,7,8,9,10-hexahydropyrido[2,3-b][1,6]naphthyridine-4-carbonitrile (5A) and (R)-3-fluoro-5,7,8,8-tetramethyl-6-oxo-5-phenyl-5,6,7,8,9,10-hexahydropyrido[2,3-b][1,6]naphthyridine-4-carbonitrile (5B)

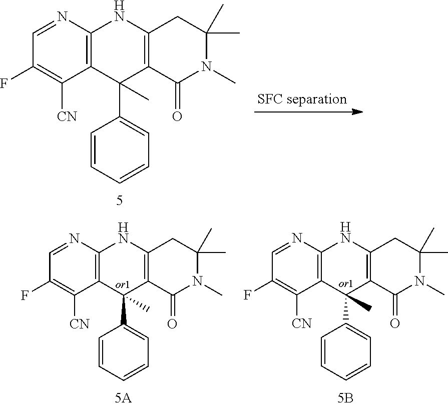

Chiral separation of rac-3-fluoro-5,7,8,8-tetramethyl-6-oxo-5-phenyl-5,6,7,8,9,10-hexahydropyrido[2,3-b][1,6]naphthyridine-4-carbonitrile (5) (55 mg, 0.146 mmol, 1.00 eq) was done in Waters SFC Prep 80 instrument equipped with Waters 2489 UV/Visible Detector by using (R,R) Whelk-O1 (21.1 mm×250 mm), 5μ Column operating at 35° C. temperature, maintaining flow rate of 70 ml/min, using 55% CO₂ in super-critical state and 45% of (100% Methanol) as Mobile phase, Run this isocratic mixture up to 15.0 minutes and also maintained the isobaric condition of 100 bar at 220 nm.

SFC fractions were collected and evaporated under reduced pressure to afford:

Peak 1: (S)-3-fluoro-5,7,8,8-tetramethyl-6-oxo-5-phenyl-5,6,7,8,9,10-hexahydropyrido[2,3-b][1,6]naphthyridine-4-carbonitrile (5A) (16.21 mg, 99.97% purity, 30% yield).

UPLC: Rt=2.25 min, (ES+) m/z (M+H)⁺=377.2.

¹H NMR (400 MHz, DMSO-D6) d 9.89 (s, 1H), 8.34 (s, 1H), 7.35 (d, J=7.6 Hz, 2H), 7.22 (t, J=7.6 Hz, 2H), 7.09 (t, J=7.2 Hz, 1H), 2.57 (s, 3H), 2.55-2.53 (m, 1H), 2.19 (s, 3H), 1.22 (s, 3H), 1.11 (s, 3H)

[α]²⁵=250.234, c=0.1243, CH3CN.

Chiral purity (ee %): 100

Peak 1: (R)-3-fluoro-5,7,8,8-tetramethyl-6-oxo-5-phenyl-5,6,7,8,9,10-hexahydropyrido[2,3-b][1,6]naphthyridine-4-carbonitrile (5B) (18.19 mg, 99.95% purity, 33% yield).

UPLC: Rt=2.25 min, (ES+) m/z (M+H)+=377.2.

¹H NMR (400 MHz, DMSO-D6) d 9.89 (s, 1H), 8.34 (s, 1H), 7.35 (d, J=7.6 Hz, 2H), 7.22 (t, J=7.6 Hz, 2H), 7.09 (t, J=7.2 Hz, 1H), 2.57 (s, 3H), 2.55-2.53 (m, 1H), 2.19 (s, 3H), 1.22 (s, 3H), 1.11 (s, 3H)

[α]²⁵=−254.538, c=0.1265, CH3CN.

Chiral purity (ee %): 99.63

### Example 3.7. (5S)-3-(difluoromethyl)-5,8,8-trimethyl-5-(o-tolyl)-6-oxo-9,10-dihydro-7H-benzo[b][1,8]naphthyridine-4-carbonitrile (14A) and (5R)-3-(difluoromethyl)-5,8,8-trimethyl-5-(o-tolyl)-6-oxo-9,10-dihydro-7H-benzo[b][1,8]naphthyridine-4-carbonitrile (14B)

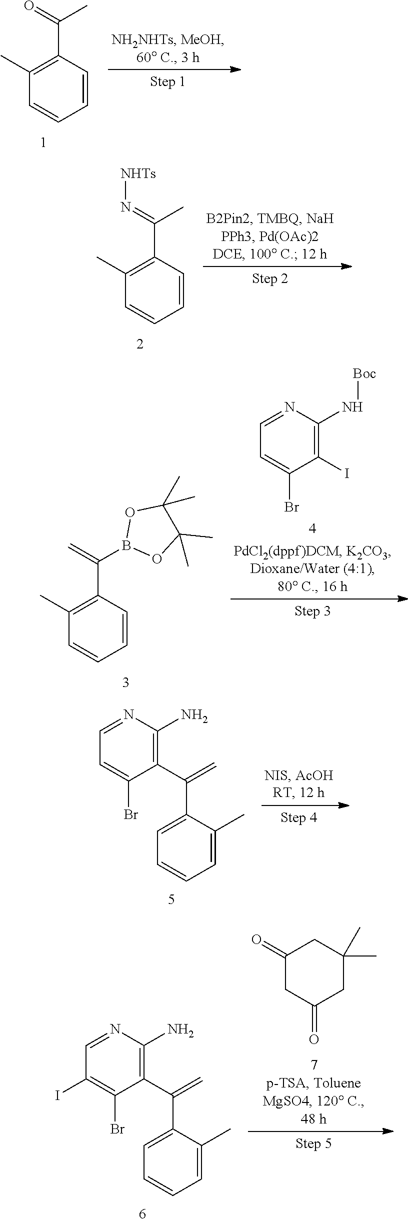

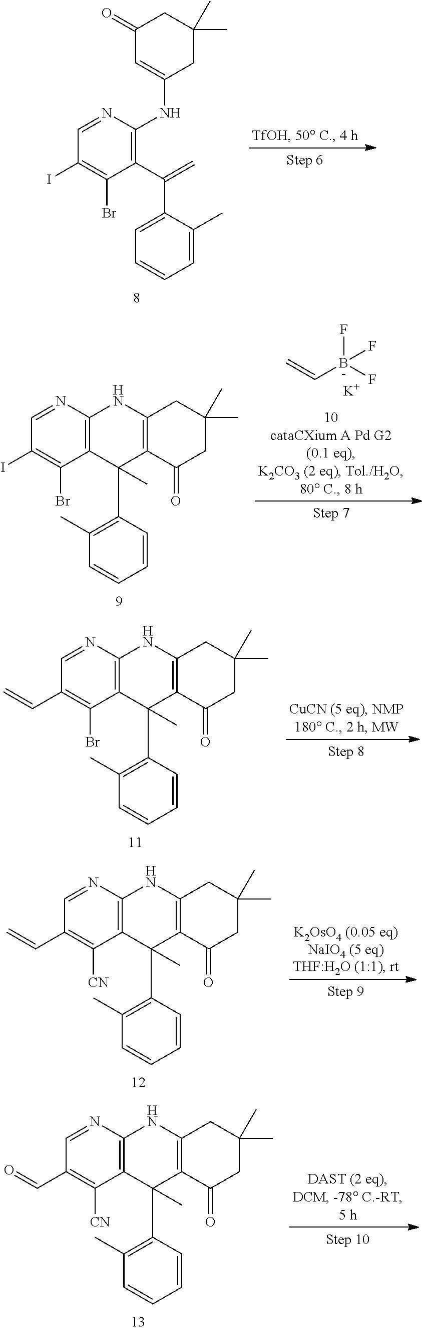

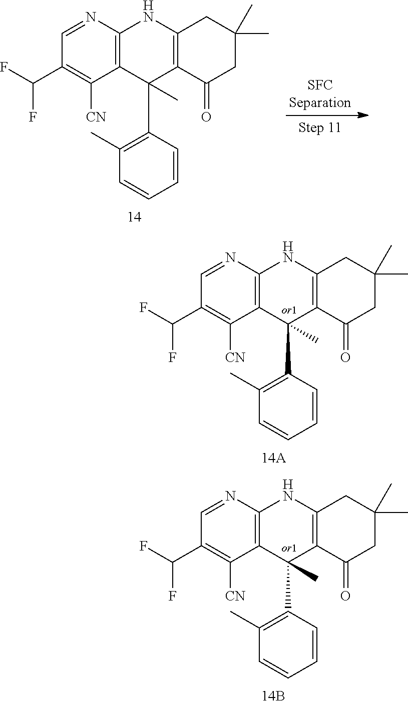 [SMILES: Cc1ccccc1C1(C)C2=C(CC(C)(C)CC2=O)Nc2ncc(C(F)F)c(C#N)c21 ; Cc1ccccc1[C@]1(C)C2=C(CC(C)(C)CC2=O)Nc2ncc(C(F)F)c(C#N)c21 ; Cc1ccccc1[C@@]1(C)C2=C(CC(C)(C)CC2=O)Nc2ncc(C(F)F)c(C#N)c21]

### Step 1: (E)-4-methyl-N′-(1-(o-tolyl)ethylidene)benzenesulfonohydrazide (2)

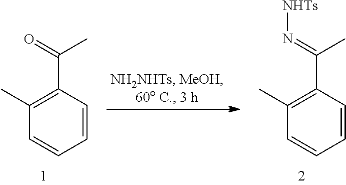

To a stirred solution of 1-(o-tolyl)ethanone (1) (2 g, 14.9 mmol, 1 eq) in Methanol (20 mL), p-toluenesulfonylhydrazide (3.53 g, 16.4 mmol, 1.1 eq) was added and the reaction mixture was then stirred at 60° C. for 3 h. After completion of the reaction, the reaction mixture was concentrated and purified by flash Combi-Flash column chromatography using EtOAc in Hexane to afford the desired compound 4-methyl-N-[(E)-1-(o-tolyl)ethylideneamino]benzenesulfonamide (2) (3.5 g, 11.2 mmol, 97% purity, 75% yield) as white solid.

LCMS: (ES⁺) m/z (M+H)+=303.2, Rt=3.46 min.

### Step 2: 4,4,5,5-tetramethyl-2-(1-(o-tolyl)vinyl)-1,3,2-dioxaborolane (3)

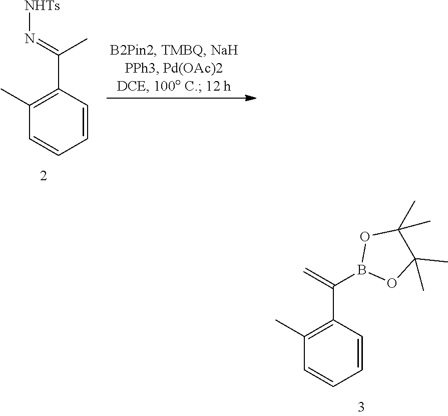

To a stirred solution of 4-methyl-N-[(E)-1-(o-tolyl)ethylideneamino]benzenesulfonamide (2) (4 g, 13.2 mmol, 1 eq) in DCE (200 mL) under Ar atmosphere were sequentially added B2Pin2 (5.02 g, 19.8 mmol, 1.5 eq), PPh3 (693 mg, 2.65 mmol, 0.2 eq) and TMBQ (2.97 g, 19.8 mmol, 1.5 eq) at RT. Resulting mixture was then degassed for 10 min followed by portion-wise addition of NaH (60% in oil) (952 mg, 39.7 mmol, 3 eq) and Pd(OAC)2 (296 mg, 1.32 mmol, 0.1 eq) at RT. Resulting mixture was then allowed to reflux for 12 h under inert atmosphere. On completion, the reaction mixture was filtered through celite bed and washed with EtOAc. Obtained filtrate was then concentrated under reduced pressure to afford a brownish liquid crude. The Crude was purified by Combi-Flash column chromatography using 0-10% EtOAc in hexane gradient to furnish 4,4,5,5-tetramethyl-2-[1-(o-tolyl)vinyl]-1,3,2-dioxaborolane (3) (840 mg, 3.44 mmol, 74% purity, 26% yield) as light yellow liquid.

LCMS: (ES+) m/z (M+H)+=245.2, Rt=4.00 min.

### Step 3: 4-bromo-3-(1-(o-tolyl)vinyl)pyridin-2-amine (5)

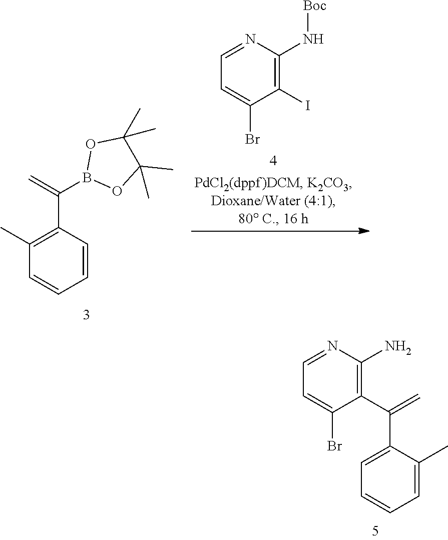

To a stirred solution of tert-butyl N-(4-bromo-3-iodo-2-pyridyl)carbamate (4) (2.5 g, 6.27 mmol, 1 eq), 4,4,5,5-tetramethyl-2-[1-(o-tolyl)vinyl]-1,3,2-dioxaborolane (3) (1.68 g, 6.89 mmol, 1.1 eq) and K2CO3 (2.16 g, 15.7 mmol, 2.5 eq) in Dioxane (20 mL) and water (4 mL) was degassed with N2 for 10 min. Then Pd(dppf)Cl₂ (511 mg, 0.627 mmol, 0.1 eq) was added and the reaction was heated at 80° C. for 16 h. After completion of the reaction, the mixture was poured into ethyl acetate, which was washed with water and brine. The combined organic layer was dried over anhydrous Na2SO4 and evaporated under vacuum to afford the crude. The crude residue was purified by Com-Flash column chromatography using ethyl acetate and hexane as the eluent to afford 4-bromo-3-[1-(o-tolyl)vinyl]pyridin-2-amine (5) (820 mg, 2.52 mmol, 89% purity, 40% yield).

LCMS: (ES+) m/z (M+H)+=289.2, Rt=3.57 min.

### Step 4: 4-bromo-5-iodo-3-(1-(o-tolyl)vinyl)pyridin-2-amine (6)

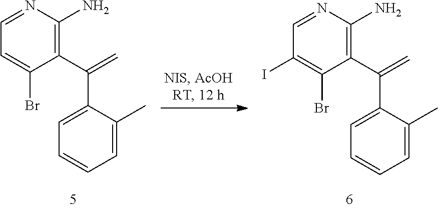 [SMILES: C=C(c1ccccc1C)c1c(Br)ccnc1N ; C=C(c1ccccc1C)c1c(N)ncc(I)c1Br]

To a stirred solution of 4-bromo-3-[1-(o-tolyl)vinyl]pyridin-2-amine (5) (300 mg, 1.04 mmol, 1 eq) in AcOH (4 mL) was added NIS (280 mg, 1.24 mmol, 1.2 eq) slowly at 0° C. under inert atmosphere. Then the reaction mass was stirred at 0° C. for 1 h. After that the reaction mixture was stirred at 25° C. for 12 h. On completion, the reaction mass was diluted with EtOAc, quenched with NaHCO3 solution and washed with sodium thiosulfate solution successively. The organic layer was then dried and concentrated under vacuum to get the crude material which was further purified by Combi-Flash column chromatography using EtOAc in hexane to afford 4-bromo-5-iodo-3-[1-(o-tolyl)vinyl]pyridin-2-amine (6) (260 mg, 0.532 mmol, 85% purity, 51% yield).

LCMS: (ES+) m/z (M+H)+=415.0, Rt=2.14 min.

### Step 5: 3-((4-bromo-5-iodo-3-(1-(o-tolyl)vinyl)pyridin-2-yl)amino)-5,5-dimethylcyclohex-2-en-1-one (8)

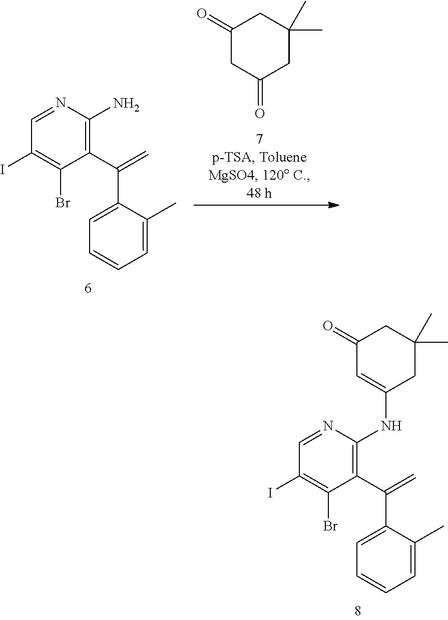

To a stirred solution of 4-bromo-5-iodo-3-[1-(o-tolyl)vinyl]pyridin-2-amine (6) (400 mg, 0.964 mmol, 1 eq) in Toluene (5 mL) were sequentially added 5,5-dimethylcyclohexane-1,3-dione (7) (176 mg, 1.25 mmol, 1.3 eq), MgSO4 (1.1 mL, 9.64 mmol, 10 eq) and PTSA (33 mg, 0.193 mmol, 0.2 eq) under inert atmosphere at RT. The resulting mixture was then allowed to reflux at 120° C. for 48 h. On completion, the reaction mass was filtered, and the obtained filtrate was evaporated under reduced pressure to furnish a blackish crude. The crude thus obtained was purified by Combi-Flash chromatography using 20-70% EtOAc-hexane gradient to furnish the desired product 3-[[4-bromo-5-iodo-3-[1-(o-tolyl)vinyl]-2-pyridyl]amino]-5,5-dimethyl-cyclohex-2-en-1-one (8) (150 mg, 0.279 mmol, 90% purity, 29% yield).

LCMS: (ES+) m/z (M+H)+=537.0, Rt=2.26 min.

### Step 6: 4-bromo-3-iodo-5,8,8-trimethyl-5-(o-tolyl)-5,8,9,10-tetrahydrobenzo[b][1,8]naphthyridin-6(7H)-one (9)

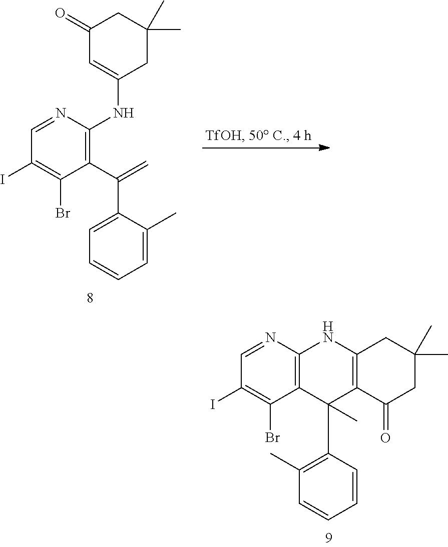 [SMILES: C=C(c1ccccc1C)c1c(NC2=CC(=O)CC(C)(C)C2)ncc(I)c1Br ; Cc1ccccc1C1(C)C2=C(CC(C)(C)CC2=O)Nc2ncc(I)c(Br)c21]

TfOH (0.21 mL, 2.33 mmol, 25.0 eq) was added to 3-[[4-bromo-5-iodo-3-[1-(o-tolyl)vinyl]-2-pyridyl]amino]-5,5-dimethyl-cyclohex-2-en-1-one (8) (50 mg, 0.093 mmol, 1 eq) at 0° C., stirred at same temperature for 0.5 h. Then the reaction mixture was heated at 50° C. for 4 h. After completion, the reaction mixture was quenched with saturated NaHCO₃ solution at 0° C., extracted with EtOAc. The organic layer was dried over Na2SO4, filtered and concentrated under reduced pressure to afford the crude. The crude thus obtained was purified by Prep TLC (eluted in 30% EtOAc in hexane) to afford 4-bromo-3-iodo-5,8,8-trimethyl-5-(o-tolyl)-9,10-dihydro-7H-benzo[b][1,8]naphthyridin-6-one (9) (30 mg, 0.056 mmol, 95% purity, 60% yield).

LCMS: (ES+) m/z (M+H)⁺=537.0, Rt=3.79 min.

### Step 7: 4-bromo-5,8,8-trimethyl-5-(o-tolyl)-3-vinyl-5,8,9,10-tetrahydrobenzo[b][1,8]naphthyridin-6(7H)-one (11)

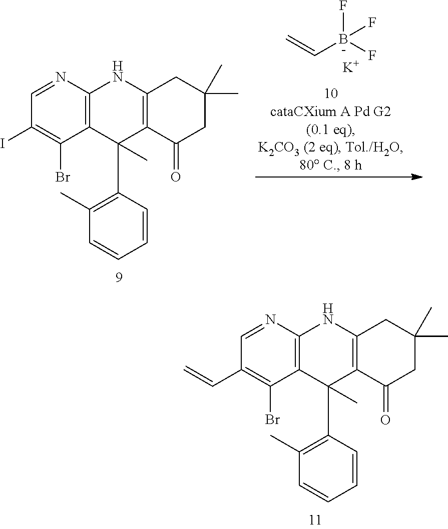

To a stirred solution of 4-bromo-3-iodo-5,8,8-trimethyl-5-(o-tolyl)-9,10-dihydro-7H-benzo[b][1,8]naphthyridin-6-one (9) (250 mg, 0.465 mmol, 1 eq) in Toluene:Water (4:1) (5 mL) were added potassium trifluoro(vinyl)borate (10) (75 mg, 0.558 mmol, 1.2 eq) and K₂CO₃ (129 mg, 0.931 mmol, 2 eq). Then the reaction mixture was purged with argon for 10 min and followed by cataCXium A Pd G2 (28 mg, 0.0419 mmol, 0.09 eq) was added. The resulting mixture was then allowed to stir at 80° C. for 8 h. After completion, the reaction mass was diluted with EtOAc, washed with water and brine. The organic layer was then dried over Na2SO4, filtered and concentrated to afford the crude which was purified by Combi-Flash column chromatography using EtOAc in hexane to afford 4-bromo-5,8,8-trimethyl-5-(o-tolyl)-3-vinyl-9,10-dihydro-7H-benzo[b][1,8]naphthyridin-6-one (11) (105 mg, 0.235 mmol, 98% purity, 51% yield).

LCMS: (ES+) m/z (M+H)⁺=437.0, Rt=3.78 min.

### Step 8: 5,8,8-trimethyl-6-oxo-5-(o-tolyl)-3-vinyl-5,6,7,8,9,10-hexahydrobenzo[b][1,8]naphthyridine-4-carbonitrile (12)

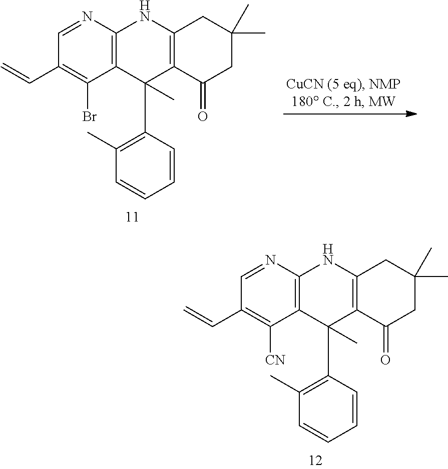 [SMILES: C=Cc1cnc2c(c1Br)C(C)(c1ccccc1C)C1=C(CC(C)(C)CC1=O)N2 ; C=Cc1cnc2c(c1C#N)C(C)(c1ccccc1C)C1=C(CC(C)(C)CC1=O)N2]

To a stirred solution of 4-bromo-5,8,8-trimethyl-5-(o-tolyl)-3-vinyl-9,10-dihydro-7H-benzo[b][1,8]naphthyridin-6-one (11) (300 mg, 0.686 mmol, 1 eq) in NMP (3 mL), CuCN (307 mg, 3.43 mmol, 5 eq) was added and stirred for 2 h at 180° C. in a microwave. After completion the reaction mixture was diluted with EtOAc and washed with water and brine, dried over sodium sulfate and concentrated. The crude thus obtained was purified by flash column chromatography using EtOAc in Hexane to afford 5,8,8-trimethyl-5-(o-tolyl)-6-oxo-3-vinyl-9,10-dihydro-7H-benzo[b][1,8]naphthyridine-4-carbonitrile (12) (165 mg, 0.43 mmol, 63% yield).

LCMS: (ES+) m/z (M+H)⁺=384.3, Rt=1.99 min.

### Step 9: 3-formyl-5,8,8-trimethyl-6-oxo-5-(o-tolyl)-5,6,7,8,9,10-hexahydrobenzo[b][1,8]naphthyridine-4-carbonitrile (13)

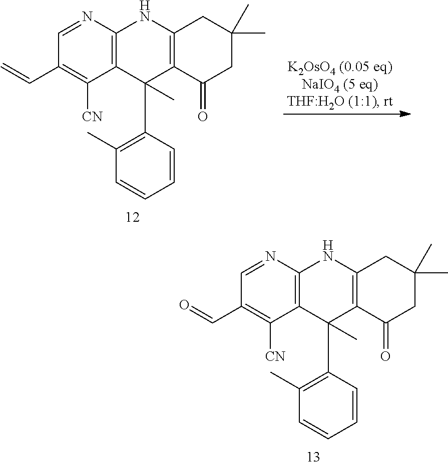 [SMILES: C=Cc1cnc2c(c1C#N)C(C)(c1ccccc1C)C1=C(CC(C)(C)CC1=O)N2 ; Cc1ccccc1C1(C)C2=C(CC(C)(C)CC2=O)Nc2ncc(C=O)c(C#N)c21]

To a stirred solution of 5,8,8-trimethyl-5-(o-tolyl)-6-oxo-3-vinyl-9,10-dihydro-7H-benzo[b][1,8]naphthyridine-4-carbonitrile (12) (220 mg, 0.574 mmol, 1 eq) in THF; H₂O (1:1) (8 mL) were added Potassium osmate (9.5 mg, 0.03 mmol, 0.05 eq) and Sodium periodate (614 mg, 2.87 mmol, 5 eq) and stirred for 3 h at 25° C. On completion, the reaction mixture was diluted with water and extracted with Ethyl Acetate, followed by brine wash, dried over Na2SO4 and concentrated under reduced pressure to afford 3-formyl-5,8,8-trimethyl-5-(o-tolyl)-6-oxo-9,10-dihydro-7H-benzo[b][1,8]naphthyridine-4-carbonitrile (13) (200 mg, 0.425 mmol, 82% purity, 74% yield) which was used for next step without purification.

LCMS: (ES+) m/z (M+H)+=386.0, Rt=3.34 min.

### Step 10: 3-(difluoromethyl)-5,8,8-trimethyl-6-oxo-5-(o-tolyl)-5,6,7,8,9,10-hexahydrobenzo[b][1,8]naphthyridine-4-carbonitrile (14)

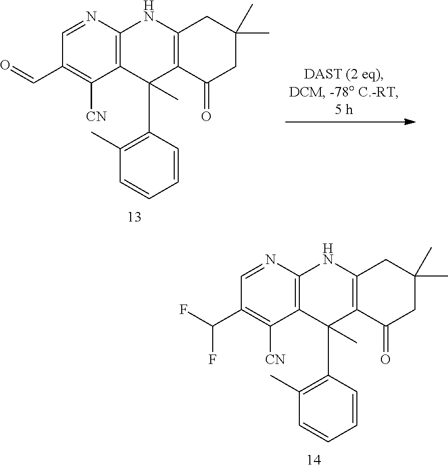 [SMILES: Cc1ccccc1C1(C)C2=C(CC(C)(C)CC2=O)Nc2ncc(C=O)c(C#N)c21 ; Cc1ccccc1C1(C)C2=C(CC(C)(C)CC2=O)Nc2ncc(C(F)F)c(C#N)c21]

To a stirred solution of 3-formyl-5,8,8-trimethyl-5-(o-tolyl)-6-oxo-9,10-dihydro-7H-benzo[b][1,8]naphthyridine-4-carbonitrile (13) (240 mg, 0.623 mmol, 1 eq) in DCM (6 mL) DAST (0.16 mL, 1.25 mmol, 2 eq) was added dropwise at −78° C. under inert atmosphere. The resulting mixture was stirred under the same condition for 0.5 h and continued for another 5 h at 25° C. On completion, the reaction mixture was quenched with saturated NaHCO₃ solution and extract with DCM, dried over Na2SO4 and concentrated under reduced pressure to afford a yellowish crude which was purified by Combi-Flash column chromatography using EtOAc in hexane to afford 3-(difluoromethyl)-5,8,8-trimethyl-5-(o-tolyl)-6-oxo-9,10-dihydro-7H-benzo[b][1,8]naphthyridine-4-carbonitrile (14) (75 mg, 0.184 mmol, 97% purity, 30% yield) which was submitted for chiral separation in SFC.

LCMS: (ES+) m/z (M+H)⁺=408.3, Rt=2.74 min.

### Step 11: (5S)-3-(difluoromethyl)-5,8,8-trimethyl-5-(o-tolyl)-6-oxo-9,10-dihydro-7H-benzo[b][1,8]naphthyridine-4-carbonitrile (14A) and (5R)-3-(difluoromethyl)-5,8,8-trimethyl-5-(o-tolyl)-6-oxo-9,10-dihydro-7H-benzo[b][1,8]naphthyridine-4-carbonitrile (14B)

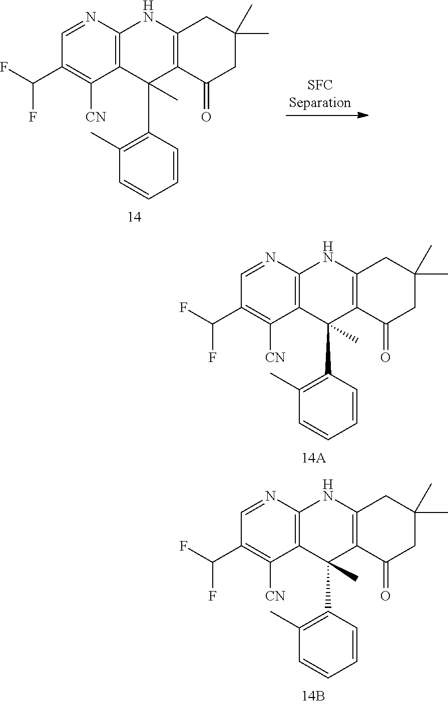 [SMILES: Cc1ccccc1C1(C)C2=C(CC(C)(C)CC2=O)Nc2ncc(C(F)F)c(C#N)c21 ; Cc1ccccc1[C@]1(C)C2=C(CC(C)(C)CC2=O)Nc2ncc(C(F)F)c(C#N)c21 ; Cc1ccccc1[C@@]1(C)C2=C(CC(C)(C)CC2=O)Nc2ncc(C(F)F)c(C#N)c21]

Chiral separation of rac-(5S)-3-(difluoromethyl)-5,8,8-trimethyl-5-(o-tolyl)-6-oxo-9,10-dihydro-7H-benzo[b][1,8]naphthyridine-4-carbonitrile (14) (75 mg, 0.184 mmol, 1 eq) was completed on Waters SFC 150 instrument equipped with Waters 2489 UV/Visible Detector by using (R,R) Whelk-O1 (30 mm×250 mm), 5μ Column operating at 35° C. temperature, maintaining flow rate of 100 ml/min, using 55% CO2 in super critical state & 45% of [100% METHANOL] as Mobile phase, Run this isocratic mixture up to 12.0 minutes and also maintained the isobaric condition of 100 bar at 210 nm wavelength.

SFC fractions were collected and evaporated under reduced pressure to afford:

Peak 1: (5S)-3-(difluoromethyl)-5,8,8-trimethyl-5-(o-tolyl)-6-oxo-9,10-dihydro-7H-benzo[b][1,8]naphthyridine-4-carbonitrile (14A) (30 mg, 0.0739 mmol, 99.56% purity, 40% yield).

UPLC: (ES+) m/z (M+H)+=408.2, Rt=2.46 mins.

¹H NMR (400 MHz, DMSO) δ 10.56 (s, 1H), 8.41 (s, 1H), 7.75 (d, J=7.56, 1H), 7.13-6.84 (m, 4H), 2.46-2.38 (m, 2H), 2.10 (s, 3H), 1.96 (d, J=7.6, 2H), 1.86 (s, 3H), 0.99 (s, 3H), 0.94 (s, 3H).

¹⁹F NMR (376 MHz, DMSO-d6) d −112.3 (d, J=54.1 Hz)

[α]²⁵=+203.848°, c=0.2315, ACN Chiral purity (ee %): 10(0%

Peak 2: (5R)-3-(difluoromethyl)-5,8,8-trimethyl-5-(o-tolyl)-6-oxo-9,10-dihydro-7H-benzo[b][1,8]naphthyridine-4-carbonitrile (14B) (30 mg, 0.0729 mmol, 98.53% purity, 40% yield).

UPLC: (ES+) m/z (M+H)+=408.2, Rt=2.46 mins.

¹H NMR (400 MHz, DMSO) δ 10.56 (s, 1H), 8.41 (s, 1H), 7.75 (d, J=7.56, 1H), 7.13-6.84 (m, 4H), 2.46-2.38 (m, 2H), 2.10 (s, 3H), 1.96 (d, J=7.6, 2H), 1.86 (s, 3H), 0.99 (s, 3H), 0.94 (s, 3H).

¹⁹F NMR (376 MHz, DMSO-d6) d −112.3 (d, J=54.1 Hz).

[α]²⁵=−204.171°, c=0.2777, ACN.

Chiral purity (ee %): 100%

### Example 3.8. (S)-5-(3-isopropoxyphenyl)-5,8,8-trimethyl-3-(trifluoromethyl)-5,8,9,10-tetrahydrobenzo[b][1,8]naphthyridin-6(7H)-one (8A) and (R)-5-(3-isopropoxyphenyl)-5,8,8-trimethyl-3-(trifluoromethyl)-5,8,9,10-tetrahydrobenzo[b][1,8]naphthyridin-6(7H)-one (8B)

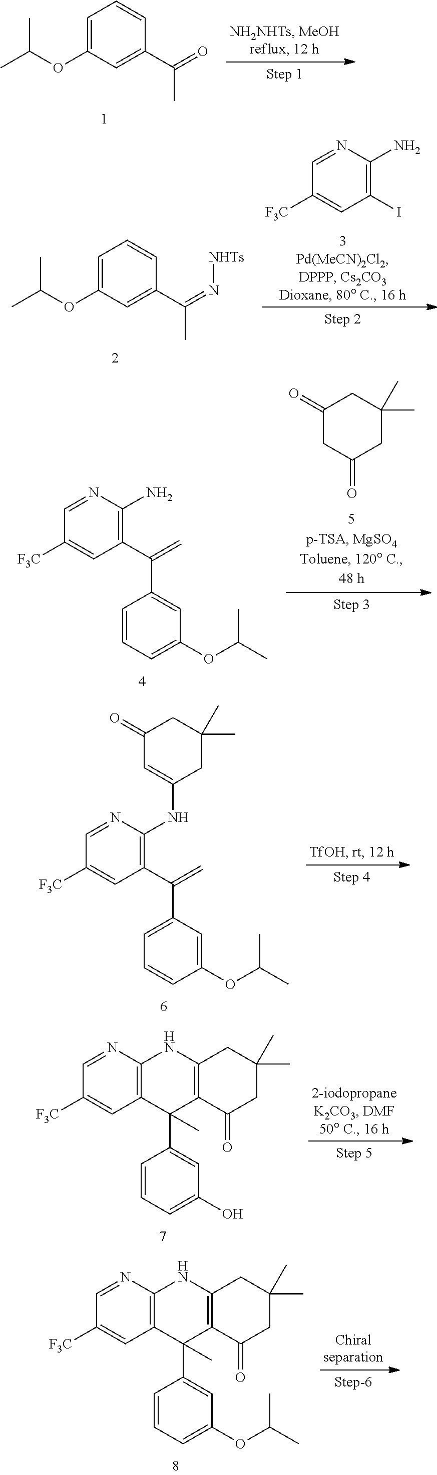

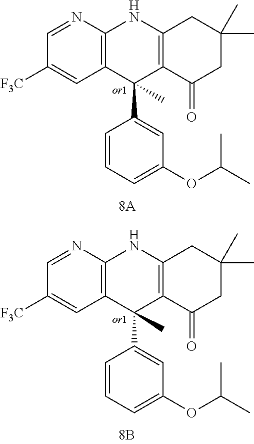 [SMILES: CC(C)Oc1cccc([C@]2(C)C3=C(CC(C)(C)CC3=O)Nc3ncc(C(F)(F)F)cc32)c1 ; CC(C)Oc1cccc([C@@]2(C)C3=C(CC(C)(C)CC3=O)Nc3ncc(C(F)(F)F)cc32)c1]

### Step 1: N—[(Z)-1-(3-isopropoxyphenyl)ethylideneamino]-4-methyl-benzenesulfonamide. (2)

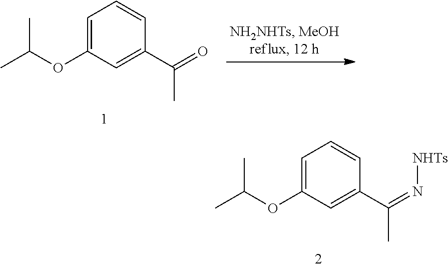

To a stirred solution of 1-(3-isopropoxyphenyl)ethenone (1) (16 g, 89.8 mmol, 1.00 eq) in MeOH (200 mL) was added p-toluenesulfonyl hydrazide (18.4 g, 98.7 mmol, 1.1 eq) and then the reaction mixture was allowed to reflux for 12 h. On completion, the reaction mixture was concentrated under reduced pressure to afford a colorless crude. Obtained crude was purified by column chromatography, eluting with 0-10% ethyl acetate in hexane gradient to afford N—[(Z)-1-(3-isopropoxyphenyl)ethylideneamino]-4-methyl-benzenesulfonamide (2) (30 g, 86.6 mmol, 96% yield) as white solid.

LCMS: (ES⁺) m/z (M+H)⁺=347.0, tR=3.62 min

### Step 2: 3-[1-(3-isopropoxyphenyl)vinyl]-5-(trifluoromethyl)pyridin-2-amine. (4)

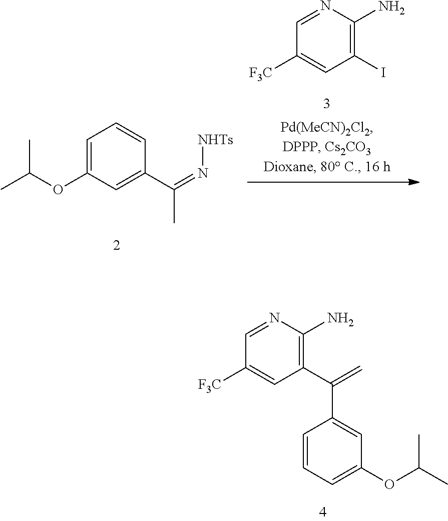

To a degassed stirred solution of 3-iodo-5-(trifluoromethyl)pyridin-2-amine (3) (1 g, 3.47 mmol, 1 eq), N—[(Z)-1-(3-isopropoxyphenyl)ethylideneamino]-4-methyl-benzenesulfonamide (2) (1.80 g, 5.21 mmol, 1.50 eq) and Cs₂CO₃ (3.39 g, 10.4 mmol, 3 eq) in Dioxane (50 mL) were sequentially added DPPP (0.29 g, 0.694 mmol, 0.2 eq) and Pd(MeCN)₂Cl₂ (0.15 g, 0.521 mmol, 0.15 eq) under inert atmosphere. The resulting mixture was allowed to stir at 80° C. for 16 h. The reaction mixture was filtered through celite pad and washed with ethyl acetate (3×20 mL). The filtrate was then washed with water (15 mL) and brine (15 mL), dried over sodium sulphate and concentrated under reduced pressure to afford a brownish crude. Obtained crude was purified by CombiFlash chromatography using 30-40% EtOAc in hexane gradient to afford 3-[1-(3-isopropoxyphenyl)vinyl]-5-(trifluoromethyl)pyridin-2-amine (4) (600 mg, 1.86 mmol, 98.6% purity, 54% yield).

LCMS: (ES⁺) m/z (M+H)⁺=323.2, tR=2.14 min

### Step 3: 3-[[3-[1-(3-isopropoxyphenyl)vinyl]-5-(trifluoromethyl)-2-pyridyl]amino]-5,5-dimethyl-cyclohex-2-en-1-one (6)

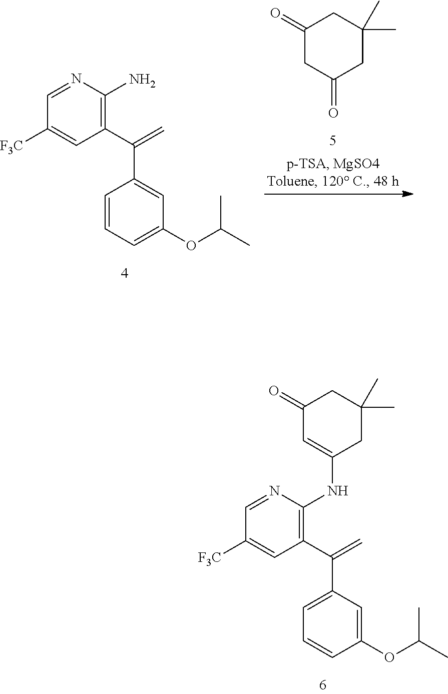

To a stirred solution of 3-[1-(3-isopropoxyphenyl)vinyl]-5-(trifluoromethyl)pyridin-2-amine (4) (450 mg, 1.40 mmol, 1 eq) and 5,5-dimethylcyclohexane-1,3-dione (5) (294 mg, 2.09 mmol, 1.5 eq) in toluene (10 mL) were sequentially added MgSO₄ (670 mg, 5.6 mmol, 4 eq) and pTSA (53 mg, 0.28 mmol, 0.2 eq) under inert atmosphere. The resulting mixture was allowed to stir at 120° C. for 48 h. On completion, reaction mass was filtered through celite pad and washed with EtOAc (3×10 mL). The combined organic phase was then concentrated under a vacuum to afford brownish crude. Crude was purified by CombiFlash chromatography using 5-10% MeOH in DCM gradient to afford 3-[[3-[1-(3-isopropoxyphenyl)vinyl]-5-(trifluoromethyl)-2-pyridyl]amino]-5,5-dimethyl-cyclohex-2-en-1-one (6) (220 mg, 0.495 mmol, 82.8% purity, 35% yield).

LCMS: (ES⁺) m/z (M+H)⁺=445.4, tR=3.88 min

### Step 4: 5-(3-hydroxyphenyl)-5,8,8-trimethyl-3-(trifluoromethyl)-9,10-dihydro-7H-benzo[b][1,8]naphthyridin-6-one (7)

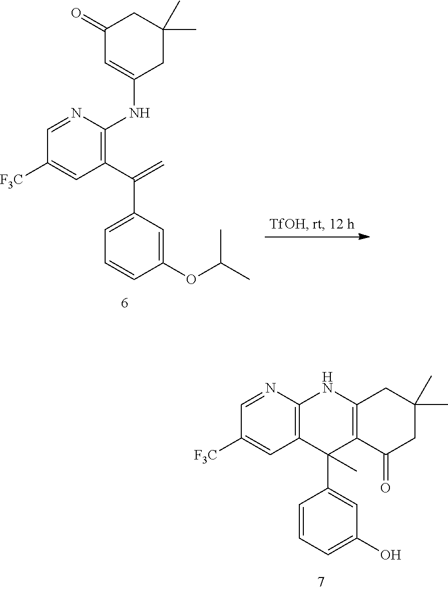 [SMILES: C=C(c1cccc(OC(C)C)c1)c1cc(C(F)(F)F)cnc1NC1=CC(=O)CC(C)(C)C1 ; CC1(C)CC(=O)C2=C(C1)Nc1ncc(C(F)(F)F)cc1C2(C)c1cccc(O)c1]

TfOH (3 mL) was dropwise added to a RB flask containing 3-[[3-[1-(3-isopropoxyphenyl)vinyl]-5-(trifluoromethyl)-2-pyridyl]amino]-5,5-dimethyl-cyclohex-2-en-1-one (6) (290 mg, 0.652 mmol, 1 eq) at 0° C. under inert atmosphere. The resulting mixture was allowed to stir at RT for 12 h. On completion, the reaction mixture was basified by chilled aqueous sodium bicarbonate and was extracted with ethyl acetate (3×15 mL). The combined organic layer was washed with brine, dried over Na₂SO₄, filtered, and concentrated under reduced pressure to afford a brownish crude. Crude was purified by CombiFlash chromatography using 60-70% ethyl acetate in hexane gradient to furnish 5-(3-hydroxyphenyl)-5,8,8-trimethyl-3-(trifluoromethyl)-9,10-dihydro-7H-benzo[b][1,8]naphthyridin-6-one (7) (120 mg, 0.298 mmol, 83.7% purity, 46% yield).

LCMS: (ES⁺) m/z (M+H)⁺=403.2, tR=1.81 min

### Step 5: 5-(3-isopropoxyphenyl)-5,8,8-trimethyl-3-(trifluoromethyl)-9,10-dihydro-7H-benzo[b][1,8]naphthyridin-6-one (8)

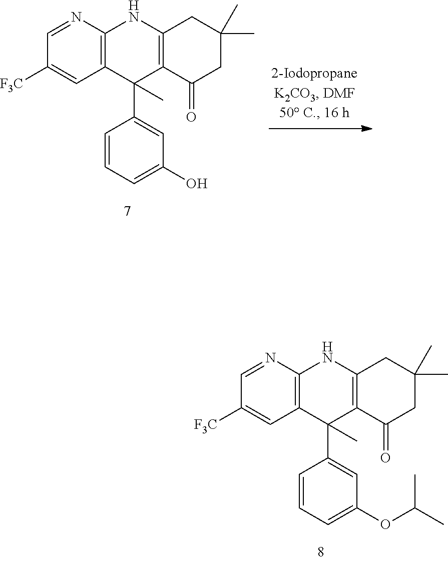 [SMILES: CC1(C)CC(=O)C2=C(C1)Nc1ncc(C(F)(F)F)cc1C2(C)c1cccc(O)c1 ; CC(C)Oc1cccc(C2(C)C3=C(CC(C)(C)CC3=O)Nc3ncc(C(F)(F)F)cc32)c1]

To a stirred solution of 5-(3-hydroxyphenyl)-5,8,8-trimethyl-3-(trifluoromethyl)-9,10-dihydro-7H-benzo[b][1,8]naphthyridin-6-one (7) (120 mg, 0.298 mmol, 1.00 eq) and K₂CO₃ (124 mg, 0.89 mmol, 3 eq) in DMF (3 mL), taken in a sealed tube, was added 2-iodopropane (0.04 mL, 0.447 mmol, 1.5 eq) under inert atmosphere and then the reaction mass was allowed to stir at 50° C. for 16 h. On completion, ice-water (10 mL) was added to the reaction mixture and was extracted with ethyl acetate (3×10 mL). The combined organic part was washed with brine (10 mL), dried over Na₂SO₄, filtered off and concentrated under reduced pressure to afford a yellowish crude. Crude was purified by CombiFlash chromatography using 40-50% ethyl acetate in hexane gradient to afford 5-(3-isopropoxyphenyl)-5,8,8-trimethyl-3-(trifluoromethyl)-9,10-dihydro-7H-benzo[b][1,8]naphthyridin-6-one (8) (70 mg, 0.157 mmol, 96.75 purity, 53% yield).

LCMS: (ES⁺) m/z (M+H)⁺=445.3, tR=2.15 min

### Step 6: Chiral Separation

### (S)-5-(3-isopropoxyphenyl)-5,8,8-trimethyl-3-(trifluoromethyl)-5,8,9,10-tetrahydrobenzo[b][1,8]naphthyridin-6(7H)-one (8A) and (R)-5-(3-isopropoxyphenyl)-5,8,8-trimethyl-3-(trifluoromethyl)-5,8,9,10-tetrahydrobenzo[b][1,8]naphthyridin-6(7H)-one (8B)

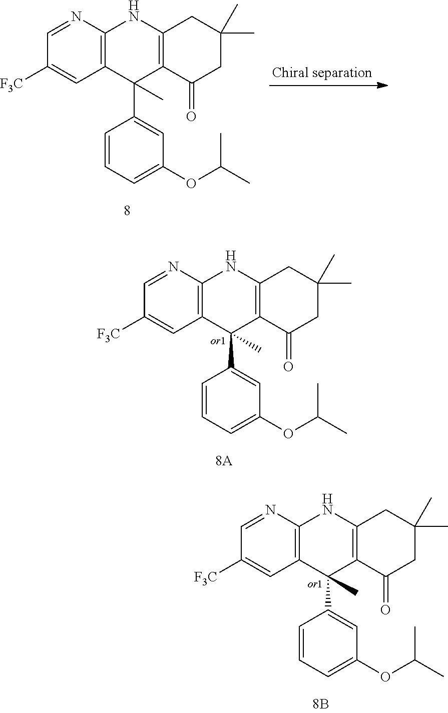 [SMILES: CC(C)Oc1cccc(C2(C)C3=C(CC(C)(C)CC3=O)Nc3ncc(C(F)(F)F)cc32)c1 ; CC(C)Oc1cccc([C@]2(C)C3=C(CC(C)(C)CC3=O)Nc3ncc(C(F)(F)F)cc32)c1 ; CC(C)Oc1cccc([C@@]2(C)C3=C(CC(C)(C)CC3=O)Nc3ncc(C(F)(F)F)cc32)c1]

Chiral separation of 5-(3-isopropoxyphenyl)-5,8,8-trimethyl-3-(trifluoromethyl)-9,10-dihydro-7H-benzo[b][1,8]naphthyridin-6-one (8) (70 mg, 0.157 mmol) was done on Waters SFC 150 instrument equipped with Waters 2489 UV/Visible Detector by using (R,R) Whelk-O1 (21.1 mm×250 mm), 5μ Column operating at 35° C. temperature, maintaining flow rate of 70 ml/min, using 60% CO₂ in super critical state & 40% of [100% Methanol] as Mobile phase. Run this isocratic mixture up to 10.0 minutes and also maintained the isobaric condition of 100 bar at 220 nm wavelength.

SFC fractions were collected and evaporated under reduced pressure to afford:

Peak 1: (S)-5-(3-isopropoxyphenyl)-5,8,8-trimethyl-3-(trifluoromethyl)-5,8,9,10-tetrahydrobenzo[b][1,8]naphthyridin-6(7H)-one (8A) (17 mg, 0.0391 mmol, 99.79% purity, 25% yield).

UPLC: Rt=2.86 min, (ES⁺) m/z (M+H)⁺=445.3.

1H NMR (400 MHz, MeOD) d 8.23 (s, 1H), 7.23 (s, 1H), 7.16 (t, 1H), 6.94 (d, 1H), 6.90 (s, 1H), 6.68 (d, 1H), 4.52 (s, 1H), 2.53 (q, 2H), 2.12 (q, 2H), 1.91 (s, 3H), 1.25 (d, 6H), 1.10 (s, 6H).

[α]²⁵=+47.999, c=0.1292, CH3CN.

Chiral purity (ee %): 100

Peak 2: (R)-5-(3-isopropoxyphenyl)-5,8,8-trimethyl-3-(trifluoromethyl)-5,8,9,10-tetrahydrobenzo[b][1,8]naphthyridin-6(7H)-one (8B) (18 mg, 0.0408 mmol, 99.71% purity, 26% yield).

UPLC: Rt=2.83 min, (ES⁺) m/z (M+H)⁺=445.3.

1H NMR (400 MHz, MeOD) d 8.23 (s, 1H), 7.23 (s, 1H), 7.16 (t, 1H), 6.94 (d, 1H), 6.90 (s, 1H), 6.68 (d, 1H), 4.52 (s, 1H), 2.53 (q, 2H), 2.12 (q, 2H), 1.91 (s, 3H), 1.25 (d, 6H), 1.10 (s, 6H).

[α]²⁵=−51.082, c=0.1292, CH3CN.

Chiral purity (ee %): 100

### Example 3.9. (S)-3-fluoro-5-(3-isopropoxyphenyl)-5,8,8-trimethyl-5,8,9,10-tetrahydrobenzo[b][1,8]naphthyridin-6(7H)-one (7A) and (R)-3-fluoro-5-(3-isopropoxyphenyl)-5,8,8-trimethyl-5,8,9,10-tetrahydrobenzo[b][1,8]naphthyridin-6(7H)-one (7B)

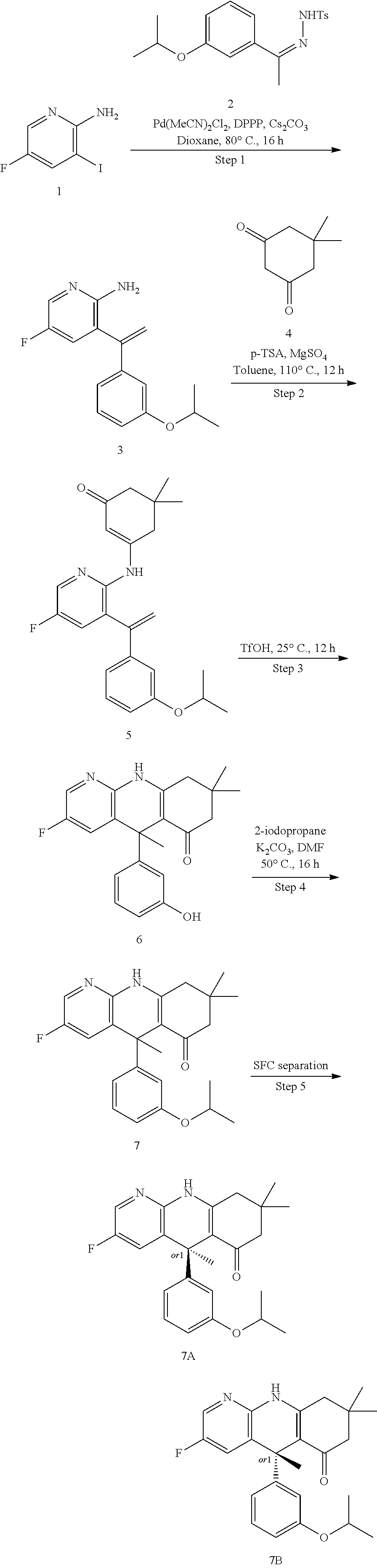

### Step 1: 5-fluoro-3-[1-(3-isopropoxyphenyl)vinyl]pyridin-2-amine. (3)

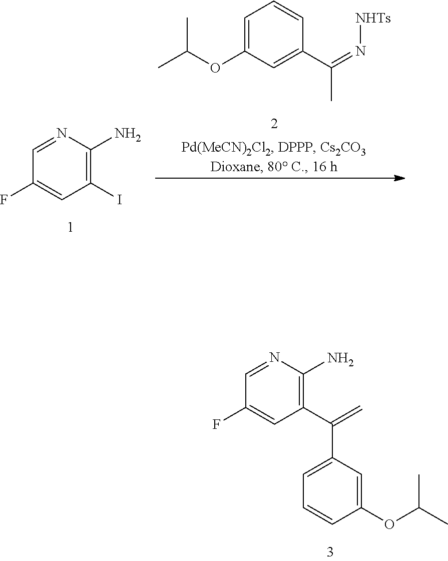

To a degassed solution of 5-fluoro-3-iodo-pyridin-2-amine (1) (1 g, 4.20 mmol, 1 eq) and N—[(Z)-1-(3-isopropoxyphenyl)ethylideneamino]-4-methyl-benzenesulfonamide (2) (2.03 g, 5.88 mmol, 1.40 eq) in Dioxane (70 mL) were sequentially added Cs₂CO₃ (4.10 g, 12.6 mmol, 3 eq), DPPP (347 mg, 0.840 mmol, 0.2 eq) and Pd(MeCN)₂Cl₂ (186 mg, 0.630 mmol, 0.15 eq) under inert atmosphere. The resulting mixture was allowed to stir at 80° C. for 16 h. The reaction mixture was filtered through celite pad and washed with ethyl acetate (3×20 mL). The combined filtrate was then washed with water (15 mL) and brine (15 mL), dried over sodium sulphate and concentrated under reduced pressure to afford a brownish crude. Obtained crude was purified by CombiFlash column chromatography using 10-30% ethyl acetate in hexane gradient to afford 5-fluoro-3-[1-(3-isopropoxyphenyl)vinyl]pyridin-2-amine (3) (500 mg, 1.84 mmol, 98.8% purity, 44% yield).

LCMS: (ES⁺) m/z (M+H)⁺=273.2, tR=2.00 min

### Step 2: 3-[[5-fluoro-3-[1-(3-isopropoxyphenyl)vinyl]-2-pyridyl]amino]-5,5-dimethyl-cyclohex-2-en-1-one (5)

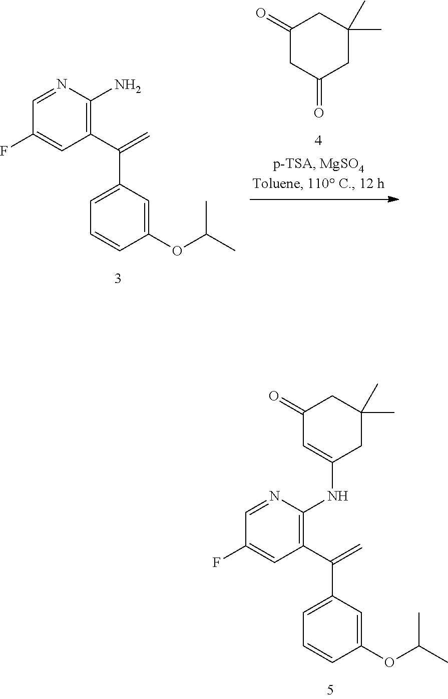

To a stirred solution of 5-fluoro-3-[1-(3-isopropoxyphenyl)vinyl]pyridin-2-amine (3) (500 mg, 1.84 mmol, 1.00 eq) and 5,5-dimethylcyclohexane-1,3-dione (4) (386 mg, 2.75 mmol, 1.50 eq) in toluene (10 mL) were sequentially added MgSO₄ (880 mg, 7.36 mmol, 4 eq) and pTSA (35 mg, 0.184 mmol, 0.1 eq) and under inert atmosphere. The resulting mixture was allowed to stir at 110° C. for 12 h. On completion, reaction mass was filtered through celite pad washed with EtOAc (20 mL). Obtained filtrate was concentrated under reduced pressure to afford a brownish crude, which was purified by CombiFlash chromatography using 5-10% MeOH in DCM gradient to afford 3-[[5-fluoro-3-[1-(3-isopropoxyphenyl)vinyl]-2-pyridyl]amino]-5,5-dimethyl-cyclohex-2-en-1-one (5) (350 mg, 0.887 mmol, 60% purity, 48% yield).

LCMS: (ES⁺) m/z (M+H)⁺=395.3, tR=2.05 min

### Step 3: 3-fluoro-5-(3-hydroxyphenyl)-5,8,8-trimethyl-9,10-dihydro-7H-benzo[b][1,8]naphthyridin-6-one (6)

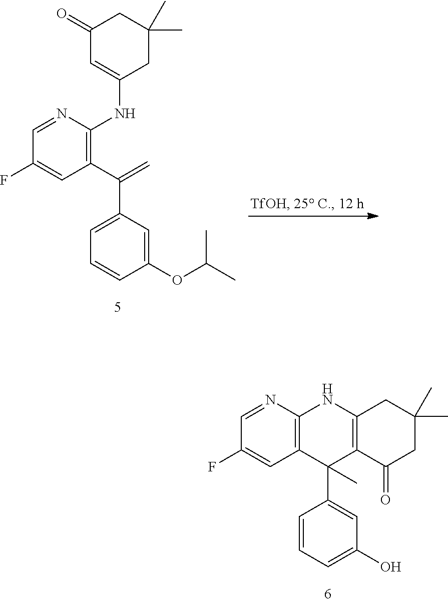 [SMILES: C=C(c1cccc(OC(C)C)c1)c1cc(F)cnc1NC1=CC(=O)CC(C)(C)C1 ; CC1(C)CC(=O)C2=C(C1)Nc1ncc(F)cc1C2(C)c1cccc(O)c1]

TfOH (3 mL) was dropwise added to a RB flask containing 3-[[5-fluoro-3-[1-(3-isopropoxyphenyl)vinyl]-2-pyridyl]amino]-5,5-dimethyl-cyclohex-2-en-1-one (5) (300 mg, 0.76 mmol, 1 eq) at 0° C. under inert atmosphere. The resulting mixture was allowed to stir at 25° C. for 12 h. On completion, the reaction mixture was basified by chilled sodium bicarbonate solution and was extracted with ethyl acetate (3×15 mL). The combined organic part was washed with brine, dried over Na₂SO₄, filtered off and concentrated to afford 3-fluoro-5-(3-hydroxyphenyl)-5,8,8-trimethyl-9,10-dihydro-7H-benzo[b][1,8]naphthyridin-6-one (6) as reddish crude. Crude was forwarded to the next step without further purification.

### Step 4: 3-fluoro-5-(3-isopropoxyphenyl)-5,8,8-trimethyl-9,10-dihydro-7H-benzo[b][1,8]naphthyridin-6-one (7)

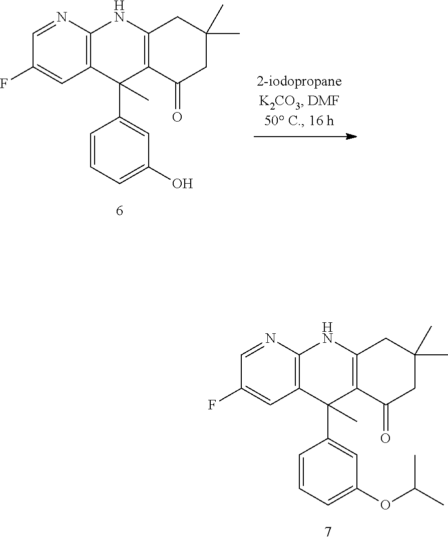 [SMILES: CC1(C)CC(=O)C2=C(C1)Nc1ncc(F)cc1C2(C)c1cccc(O)c1 ; CC(C)Oc1cccc(C2(C)C3=C(CC(C)(C)CC3=O)Nc3ncc(F)cc32)c1]

To a stirred solution of 3-fluoro-5-(3-hydroxyphenyl)-5,8,8-trimethyl-9,10-dihydro-7H-benzo[b][1,8]naphthyridin-6-one (6) (80 mg, 0.227 mmol, 1.00 eq) and K₂CO₃ (95 mg, 0.681 mmol, 3.00 eq) in DMF (2 mL), taken in a sealed tube, was added 2-iodopropane (0.03 mL, 0.341 mmol, 1.5 eq) and allowed to stir at 50° C. for 16 h. On completion, the reaction mixture was diluted with water and was extracted with ethyl acetate (3×10 mL). The combined organic part was washed with brine, dried over Na₂SO₄, filtered off and concentrated under vacuum to afford a pale yellowish crude. Obtained crude was purified by CombiFlash chromatography using 30-40% ethyl acetate in hexane gradient to furnish 3-fluoro-5-(3-isopropoxyphenyl)-5,8,8-trimethyl-9,10-dihydro-7H-benzo[b][1,8]naphthyridin-6-one (7) (55 mg, 0.139 mmol, 87.4% purity, 61% yield).

LCMS: (ES⁺) m/z (M+H)⁺=395.0, tR=3.50 min

### Step 5: Chiral Separation

### (S)-3-fluoro-5-(3-isopropoxyphenyl)-5,8,8-trimethyl-5,8,9,10-tetrahydrobenzo[b][1,8]naphthyridin-6(7H)-one (7A) and (R)-3-fluoro-5-(3-isopropoxyphenyl)-5,8,8-trimethyl-5,8,9,10-tetrahydrobenzo[b][1,8]naphthyridin-6(7H)-one (7B)

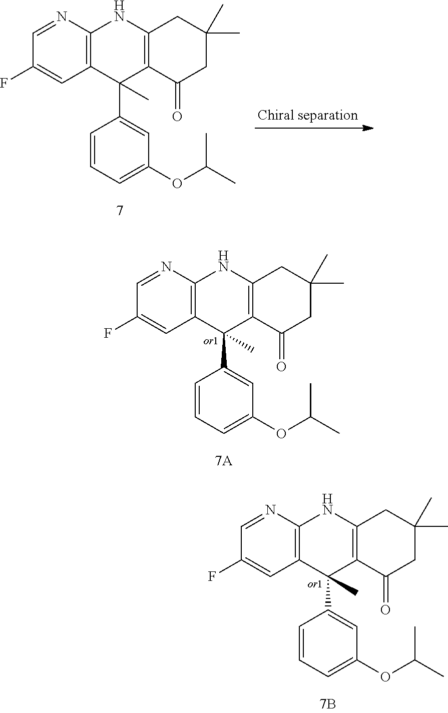 [SMILES: CC(C)Oc1cccc(C2(C)C3=C(CC(C)(C)CC3=O)Nc3ncc(F)cc32)c1 ; CC(C)Oc1cccc([C@]2(C)C3=C(CC(C)(C)CC3=O)Nc3ncc(F)cc32)c1 ; CC(C)Oc1cccc([C@@]2(C)C3=C(CC(C)(C)CC3=O)Nc3ncc(F)cc32)c1]

Chiral separation of 3-fluoro-5-(3-isopropoxyphenyl)-5,8,8-trimethyl-9,10-dihydro-7H-benzo[b][1,8]naphthyridin-6-one (7) (55 mg, 0.139 mmol) was done on SFC-80 instrument equipped with waters 2489 UV/VISIBLE detector by using (R,R) Whelk-O1 column (21.1 mm×250 mm), 5μ Column operating at 35° C. temperature, maintaining flow rate of 60 ml/min, using 60% CO2 in super critical state & 40% [100% Methanol] as Mobile phase, Run this isocratic mixture up to 10.0 minutes and also maintained the isobaric condition of 100 bar at 337 nm wavelength.

SFC fractions were collected and evaporated under reduced pressure to afford:

Peak 1: (S)-3-fluoro-5-(3-isopropoxyphenyl)-5,8,8-trimethyl-5,8,9,10-tetrahydrobenzo[b][1,8] naphthyridin-6(7H)-one (7A) (14 mg, 0.0366 mmol, 99.7% purity, 26% yield).

UPLC: Rt=2.66 min, (ES⁺) m/z (M+H)⁺=395.3.

1H NMR (400 MHz, DMSO-d6) δ 9.86 (s, 1H), 7.96 (d, 1H), 7.10 (t, 1H), 7.06-7.04 (m, 1H), 6.86-6.82 (m, 2H), 6.63 (d, 11H), 4.54-4.51 (m, 1H), 2.48 (s, 2H), 2.06-1.93 (m, 2H), 1.89 (s, 3H), 1.22 (d, 6H), 1.00 (s, 6H).

[α]²⁵=+56.898, c=0.1318, MeOH.

Chiral purity (ee %): 100

Peak 2: (R)-3-fluoro-5-(3-isopropoxyphenyl)-5,8,8-trimethyl-5,8,9,10-tetrahydrobenzo[b][1,8] naphthyridin-6(7H)-one (7B) (15 mg, 0.0374 mmol, 99.66% purity, 27% yield).

UPLC: Rt=2.66 min, (ES⁺) m/z (M+H)⁺=395.3.

1H NMR (400 MHz, DMSO-d6) d 9.86 (s, 1H), 7.96 (d, 1H), 7.10 (t, 1H), 7.06-7.04 (m, 1H), 6.86-6.82 (m, 2H), 6.63 (d, 1H), 4.54-4.51 (m, 1H), 2.48 (s, 2H), 2.06-1.93 (m, 2H), 1.89 (s, 3H), 1.22 (d, 6H), 1.00 (s, 6H).

[α]²⁵=−58.710, c=0.1312, MeOH.

Chiral purity (ee %): 100

### Example 3.10. (S)-3-fluoro-5-(3-isopropoxyphenyl)-5,8,8-trimethyl-6-oxo-5,6,7,8,9,10-hexahydrobenzo[b][1,8]naphthyridine-4-carbonitrile (10A) and (R)-3-fluoro-5-(3-isopropoxyphenyl)-5,8,8-trimethyl-6-oxo-5,6,7,8,9,10-hexahydrobenzo[b][1,8]naphthyridine-4-carbonitrile (10B)

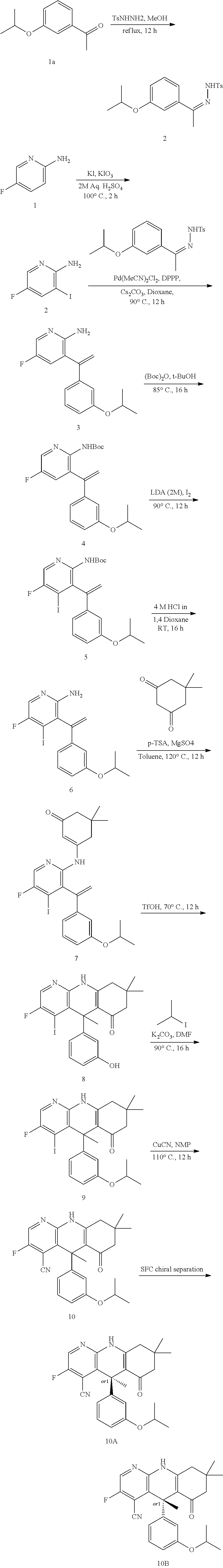

### Step 1: 5-fluoro-3-iodo-pyridin-2-amine (2)

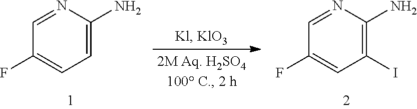 [SMILES: Nc1ccc(F)cn1 ; Nc1ncc(F)cc1I]

To a stirred solution of 5-fluoropyridin-2-amine (10.00 g, 89.2 mmol, 1.00 eq) in aqueous 2 M H₂SO₄ solution (133 mL), was slowly added potassium iodate (9.54 g, 44.6 mmol, 0.50 eq). Reaction mixture was allowed to warm at 100° C. and a solution of potassium iodide (15 g, 89.2 mmol, 1.00 eq) in H₂O (67 mL) was added to the reaction mixture under same condition. The resulting mixture was allowed to stir for another 2 h. On completion, the reaction mixture was quenched with aqueous NaHCO₃ solution to maintain pH 8-9 and extracted with ethyl acetate (twice). The combined organic layer was washed sequentially with aqueous Na₂S₂O₃, water and brine. Obtained organic part was dried over Na₂SO₄, filtered and concentrated under reduced pressure to afford 5-fluoro-3-iodo-pyridin-2-amine (12 g, 50.4 mmol, 57% yield) as off white solid.

¹H NMR (400 MHz, DMSO-d6) δ=7.96-7.93 (m, 2H), 5.98 (s, 2H).

### Step 2: 5-fluoro-3-[1-(3-isopropoxyphenyl)vinyl]pyridin-2-amine (3)

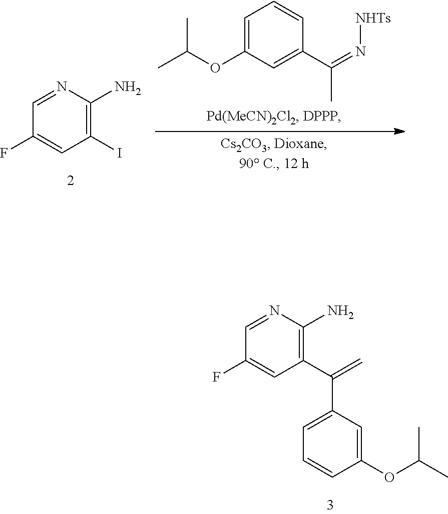

To a degassed solution of 5-fluoro-3-iodo-pyridin-2-amine (2) (6 g, 25.2 mmol, 1.0 eq), N—[(Z)-1-(3-isopropoxyphenyl)ethylideneamino]-4-methyl-benzenesulfonamide (12.2 g, 35.3 mmol, 1.4 eq) and Cs₂CO₃ (25 g, 75.6 mmol, 3.0 eq) in Dioxane (500 mL) were sequentially added DPPP (2 g, 5.04 mmol, 0.2 eq) and Pd(MeCN)₂Cl₂ (1.12 g, 3.78 mmol, 0.15 eq) at RT under inert atmosphere. The resulting mixture was allowed to stir at 90° C. for 12 h. RM was filtered over celite pad and washed with ethyl acetate (twice). The filtrate part was washed with water, brine, dried over Na₂SO₄ and concentrated under reduced pressure to afford a brownish crude. Obtained crude was purified by CombiFlash chromatography using 7-9% ethylacetate-hexane gradient to afford 5-fluoro-3-[1-(3-isopropoxyphenyl)vinyl]pyridin-2-amine (3) (3.50 g, 12.5 mmol, 97% purity, 49% yield) as off white solid.

LCMS: Rt=2.01 min. (ES⁺) m/z (M+H)⁺=273.2.

### Step 3: tert-butyl N-[5-fluoro-3-[1-(3-isopropoxyphenyl)vinyl]-2-pyridyl]carbamate. (4)

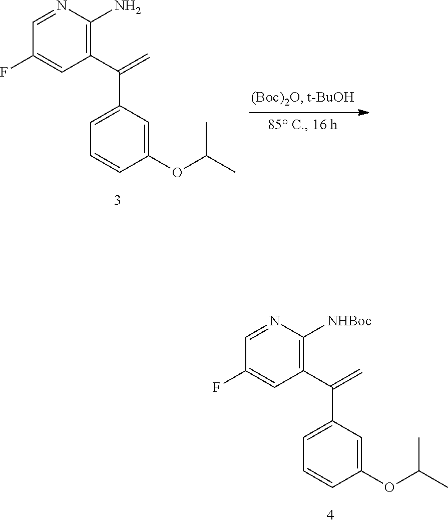 [SMILES: C=C(c1cccc(OC(C)C)c1)c1cc(F)cnc1N ; C=C(c1cccc(OC(C)C)c1)c1cc(F)cnc1NC(=O)OC(C)(C)C]

To a stirred solution of 5-fluoro-3-[I-(3-isopropoxyphenyl)vinyl]pyridin-2-amine (3) (3.50 g, 12.9 mmol, 1 eq) in t-BuOH (20 mL) was dropwise added Boc₂O (7.4 mL, 32.1 mmol, 2.5 eq) and the resulting mixture was allowed to stir at 85° C. for 16 h. On completion, the reaction mixture was diluted with ethyl acetate and was washed sequentially with water and brine, dried over anhydrous Na₂SO₄, filtered and concentrated under reduced pressure to afford a light yellowish crude. Crude was purified by CombiFlash column chromatography using 5%-15% ethylacetate-hexane gradient to afford tert-butyl N-[5-fluoro-3-[1-(3-isopropoxyphenyl)vinyl]-2-pyridyl]carbamate (4) (2.80 g, 7.07 mmol, 94% purity, 55% yield) as off white solid.

LCMS: Rt=2.16 min, (ES⁺) m/z (M+H)⁺=373.2.

### Step 4: tert-butyl N-[5-fluoro-4-iodo-3-[1-(3-isopropoxyphenyl)vinyl]-2-pyridyl]carbamate. (5)

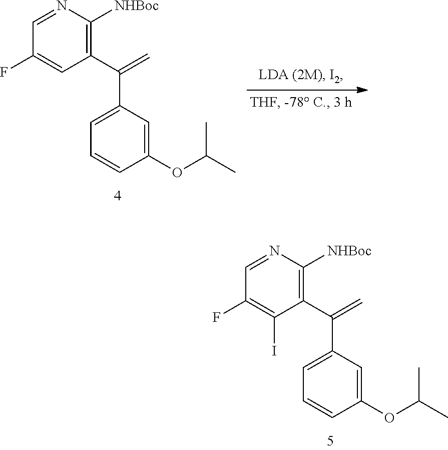 [SMILES: C=C(c1cccc(OC(C)C)c1)c1cc(F)cnc1NC(=O)OC(C)(C)C ; C=C(c1cccc(OC(C)C)c1)c1c(NC(=O)OC(C)(C)C)ncc(F)c1I]

To a stirred solution of tert-butyl N-[5-fluoro-3-[1-(3-isopropoxyphenyl)vinyl]-2-pyridyl]carbamate (4) (2.80 g, 7.52 mmol, 1.00 eq) in THF (50 mL) was dropwise added LDA (2 M in hexane, 13.2 mL, 26.3 mmol, 3.5 eq) dropwise at −78° C., stirred the reaction mixture for 1 h at the same temperature. Then Iodine (2.85 g, 11.3 mmol, 1.50 eq) in THF (25 mL) was dropwise added and the resulting mixture was allowed to stir for another 1 h under same condition. Reaction mixture was allowed to stir at 25° C. for 1 h, quenched with aqueous NH₄Cl solution and extracted with ethyl acetate. The ethyl acetate part was washed with aqueous Na₂S₂O₃ solution, brine, dried over anhydrous Na₂SO₄, filtered and concentrated under reduced pressure to obtain brownish crude. Crude was purified by CombiFlash chromatography using 5-10% ethyl acetate in hexane gradient to furnish tert-butyl-N-[5-fluoro-4-iodo-3-[1-(3-isopropoxyphenyl)vinyl]-2-pyridyl]carbamate (5) (1.50 g, 3.01 mmol, 40% yield) as off white solid.

LCMS: Rt=2.17 min, (ES⁺) m/z (M+H)⁺=499.0.

### Step 5: 5-fluoro-4-iodo-3-[1-(3-isopropoxyphenyl)vinyl]pyridin-2-amine (6)

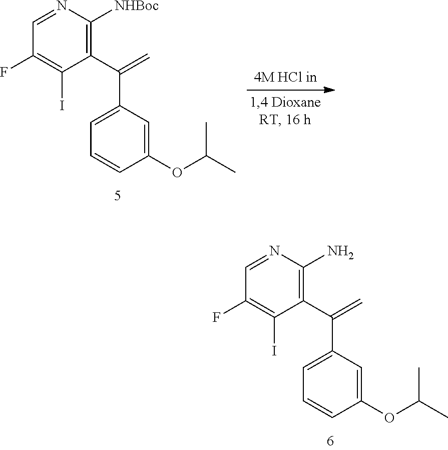 [SMILES: C=C(c1cccc(OC(C)C)c1)c1c(NC(=O)OC(C)(C)C)ncc(F)c1I ; C=C(c1cccc(OC(C)C)c1)c1c(N)ncc(F)c1I]

To a stirred solution of tert-butyl N-[5-fluoro-4-iodo-3-[1-(3-isopropoxyphenyl)vinyl]-2-pyridyl]carbamate (5) (1.90 g, 3.81 mmol, 1.00 eq) in 1,4-Dioxane (5 mL) was drop wise added 4 M HCl in 1,4 Dioxane (20 mL) at 0° C. under inert atmosphere. On completion, reaction mixture was allowed to stir at RT for 16 h. Then reaction mixture was quenched with aqueous NaHCO₃ solution and extracted with Ethyl acetate. The ethyl acetate part was washed with brine, dried over Na₂SO₄, filtered and concentrated under reduced pressure to afford crude 5-fluoro-4-iodo-3-[1-(3-isopropoxyphenyl)vinyl]pyridin-2-amine (6) (1.30 g, 2.51 mmol, 77% purity, 66% yield) as brownish solid. Obtained crude was used as such for next step without further purification.

LCMS: Rt=1.75 min, (ES⁺) m/z (M+H)⁺=398.8.

### Step 6: 3-[[5-fluoro-4-iodo-3-[1-(3-isopropoxyphenyl)vinyl]-2-pyridyl]amino]-5,5-dimethyl-cyclohex-2-en-1-one (7)

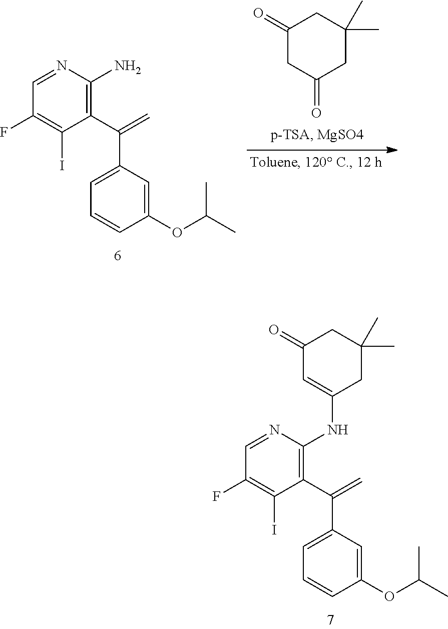

To the stirred solution of 5-fluoro-4-iodo-3-[1-(3-isopropoxyphenyl)vinyl]pyridin-2-amine (6) (1.30 g, 3.26 mmol, 1.00 eq) in Toluene (15 mL) were sequentially added 5,5-dimethylcyclohexane-1,3-dione (686 mg, 4.90 mmol, 1.50 eq), MgSO4 (4 g, 32.6 mmol, 10.0 eq) and pTSA (62 mg, 0.326 mmol, 0.100 eq) under inert atmosphere at RT. Resulting mixture was allowed to reflux for 12 h. On completion, the reaction mixture was filtered over celite pad and was washed with ethyl acetate twice. Obtained filtrate was concentrated under reduced pressure to afford a reddish crude. Crude was purified by CombiFlash column chromatography to furnish 3-[[5-fluoro-4-iodo-3-[1-(3-isopropoxyphenyl)vinyl]-2-pyridyl]amino]-5,5-dimethyl-cyclohex-2-en-1-one (7) (1.20 g, 2.17 mmol, 94% purity, 66% yield) as gummy solid.

LCMS: Rt=2.38 min, (ES⁺) m/z (M+H)⁺=521.2.

### Step 7: 3-fluoro-5-(3-hydroxyphenyl)-4-iodo-5,8,8-trimethyl-9,10-dihydro-7H-benzo[b][1,8] naphthyridin-6-one. (8)

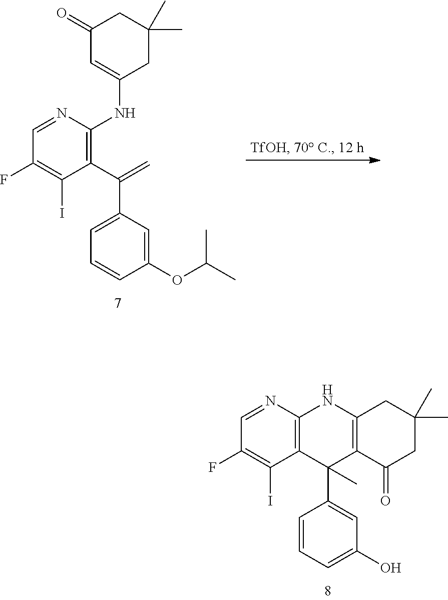 [SMILES: C=C(c1cccc(OC(C)C)c1)c1c(NC2=CC(=O)CC(C)(C)C2)ncc(F)c1I ; CC1(C)CC(=O)C2=C(C1)Nc1ncc(F)c(I)c1C2(C)c1cccc(O)c1]

TfOH (10 mL) was dropwise added to a RB containing 3-[[5-fluoro-4-iodo-3-[1-(3-isopropoxyphenyl)vinyl]-2-pyridyl]amino]-5,5-dimethyl-cyclohex-2-en-1-one (7) (1.20 g, 2.31 mmol, 1.00 eq) under inert atmosphere at 0° C. Resulting mixture was allowed to stir at 70° C. for 12 h. On completion, the reaction mixture was cooled to RT, quenched with aqueous NaHCO₃ solution and extracted with ethyl acetate (twice). The combined organic part was washed with brine, dried over Na₂SO₄, filtered and concentrated to afford a brownish crude. Obtained crude was purified by CombiFlash chromatography using 25% to 50% ethyl acetate-hexane gradient to furnish 3-fluoro-5-(3-hydroxyphenyl)-4-iodo-5,8,8-trimethyl-9,10-dihydro-7H-benzo[b][1,8]naphthyridin-6-one (8) (650 mg, 1.30 mmol, 96% purity, 57% yield) as yellow solid.

LCMS: Rt=1.83 min, (ES⁺) m/z (M+H)⁺=479.2

### Step 8: 3-fluoro-4-iodo-5-(3-isopropoxyphenyl)-5,8,8-trimethyl-9,10-dihydro-7H-benzo[b][1,8]naphthyridin-6-one. (9)

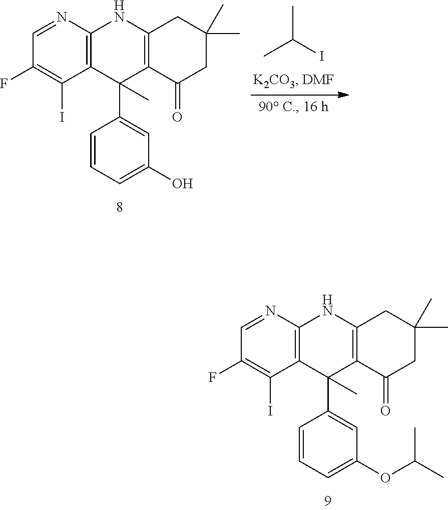

To a stirred solution of 3-fluoro-5-(3-hydroxyphenyl)-4-iodo-5,8,8-trimethyl-9,10-dihydro-7H-benzo[b][1,8]naphthyridin-6-one (8) (650 mg, 1.36 mmol, 1.00 eq) in DMF (5 mL) were sequentially added K₂CO₃ (560 mg, 4.08 mmol, 3 eq) and 2-iodopropane (0.27 mL, 2.72 mmol, 2.00 eq) under inert atmosphere at RT. Resulting mixture was allowed to stir at 90° C. for 16 h. On completion, the reaction mixture was diluted with cold water, extracted with ethyl acetate. The ethyl acetate part was washed with brine, dried over anhydrous Na₂SO₄, filtered and concentrated under reduced pressure to afford a brownish crude. Crude was purified by CombiFlash chromatography using 10-35% EA-hexane gradient to afford 3-fluoro-4-iodo-5-(3-isopropoxyphenyl)-5,8,8-trimethyl-9,10-dihydro-7H-benzo[b][1,8]naphthyridin-6-one (9) (300 mg, 0.553 mmol, 96% purity, 41% yield) as off white solid.

LCMS: Rt=2.12 min, (ES⁺) m/z (M+H)⁺=521.2.

### Step 9: 3-fluoro-5-(3-isopropoxyphenyl)-5,8,8-trimethyl-6-oxo-9,10-dihydro-7H-benzo[b][1,8]naphthyridine-4-carbonitrile (10)

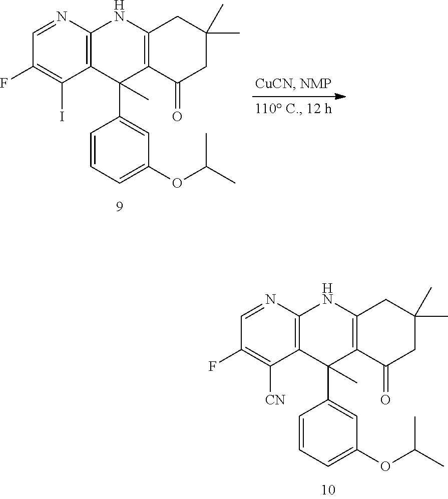 [SMILES: CC(C)Oc1cccc(C2(C)C3=C(CC(C)(C)CC3=O)Nc3ncc(F)c(I)c32)c1 ; CC(C)Oc1cccc(C2(C)C3=C(CC(C)(C)CC3=O)Nc3ncc(F)c(C#N)c32)c1]

NMP (2.5 mL) and CuCN (200 mg, 2.31 mmol, 4 eq) were sequentially added to a sealed tube containing 3-fluoro-4-iodo-5-(3-isopropoxyphenyl)-5,8,8-trimethyl-9,10-dihydro-7H-benzo [b][1,8]naphthyridin-6-one (9) (300 mg, 0.576 mmol, 1.00 eq). The resulting mixture was allowed to stir at 90° C. for 12 h. On completion, the reaction mixture was diluted with water and extracted with ethyl acetate (twice). Combined organic layer was washed with brine, dried over anhydrous Na₂SO₄, filtered and concentrated under reduced pressure to afford a yellowish crude. Crude was purified by CombiFlash chromatography using 10-12% ethyl acetate in hexane gradient to furnish 3-fluoro-5-(3-isopropoxyphenyl)-5,8,8-trimethyl-6-oxo-9,10-dihydro-7H-benzo[b] [1,8]naphthyridine-4-carbonitrile (10) (130 mg, 0.310 mmol, 100% purity, 54% yield) as light yellow solid.

LCMS: Rt=3.56 min, (ES⁺) m/z (M+H)⁺=420.2.

### Step 10: Chiral Separation

### (S)-3-fluoro-5-(3-isopropoxyphenyl)-5,8,8-trimethyl-6-oxo-5,6,7,8,9,10-hexahydrobenzo[b][1,8]naphthyridine-4-carbonitrile (10A) and (R)-3-fluoro-5-(3-isopropoxyphenyl)-5,8,8-trimethyl-6-oxo-5,6,7,8,9,10-hexahydrobenzo[b][1,8]naphthyridine-4-carbonitrile (10B)

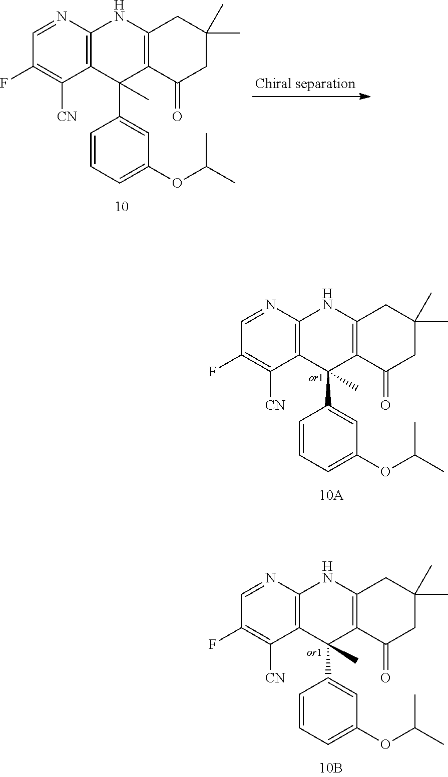 [SMILES: CC(C)Oc1cccc(C2(C)C3=C(CC(C)(C)CC3=O)Nc3ncc(F)c(C#N)c32)c1 ; CC(C)Oc1cccc([C@]2(C)C3=C(CC(C)(C)CC3=O)Nc3ncc(F)c(C#N)c32)c1 ; CC(C)Oc1cccc([C@@]2(C)C3=C(CC(C)(C)CC3=O)Nc3ncc(F)c(C#N)c32)c1]

Chiral separation of 3-fluoro-5-(3-isopropoxyphenyl)-5,8,8-trimethyl-6-oxo-9,10-dihydro-7H-benzo[b] [1,8]naphthyridine-4-carbonitrile (10) (130 mg, 0.31 mmol) was done by SFC 80 instrument equipped with Waters 2489 UV/Visible detector by using Reflect (R,R)Whelk_O1 (21.1 mm×250 mm), 5μ Column operating at 35° C. temperature, maintaining flow rate of 60 ml/min, using 60% CO2 in super critical state & 40% [100% Methanol] as Mobile phase. Run this isocratic mixture up to 7.0 minutes and also maintained the isobaric condition of 100 bar at nm wavelength.

SFC fractions were collected and evaporated under reduced pressure to afford:

Peak 1: (S)-3-fluoro-5-(3-isopropoxyphenyl)-5,8,8-trimethyl-6-oxo-5,6,7,8,9,10-hexahydrobenzo[b][1,8]naphthyridine-4-carbonitrile (10A) (54 mg, 0.128 mmol, 99.83% purity, 41% yield) as yellow solid.

UPLC: Rt=2.61 min, (ES⁺) m/z (M+H)⁺=420.2.

¹H NMR (400 MHz, DMSO-d6) δ 10.22 (s, 1H), 8.39 (s, 1H), 7.12-7.08 (m, 1H), 6.91 (s, 1H), 7.80 (d, J=7.6 Hz, 1H), 6.67 (dd, J=8, 1.6 Hz, 1H), 4.54-4.50 (m, 1H), 2.44 (s, 2H), 2.11 (s, 3H), 2.02-1.88 (m, 2H), 1.24-1.22 (m, 6H), 0.99 (s, 3H), 0.92 (s, 3H).

[α]²⁵=+258.532, c=0.2143, MeOH.

Chiral purity (ee %): 100

Peak 2: (R)-3-fluoro-5-(3-isopropoxyphenyl)-5,8,8-trimethyl-6-oxo-5,6,7,8,9,10-hexahydrobenzo[b][1,8]naphthyridine-4-carbonitrile (10B) (64 mg, 0.153 mmol, 99.86% purity, 49% yield) as yellow solid.

UPLC: Rt=2.61 min, (ES⁺) m/z (M+H)⁺=420.2.

¹H NMR (400 MHz, DMSO-d6) δ 10.22 (s, 1H), 8.39 (s, 1H), 7.12-7.08 (m, 1H), 6.91 (s, 1H), 7.80 (d, J=7.6 Hz, 1H), 6.67 (dd, J=8, 1.6 Hz, 1H), 4.54-4.50 (m, 1H), 2.44 (s, 2H), 2.11 (s, 3H), 2.02-1.88 (m, 2H), 1.24-1.22 (m, 6H), 0.99 (s, 3H), 0.92 (s, 3H).

[α]²⁵=−247.333, c=0.2143, MeOH.

Chiral purity (ee %): 100

### Example 3.11. (S)-3-(difluoromethyl)-5-(3-isopropoxyphenyl)-5,8,8-trimethyl-6-oxo-5,6,7,8,9,10-hexahydrobenzo[b][1,8]naphthyridine-4-carbonitrile (16A) and (R)-3-(difluoromethyl)-5-(3-isopropoxyphenyl)-5,8,8-trimethyl-6-oxo-5,6,7,8,9,10-hexahydrobenzo[b][1,8]naphthyridine-4-carbonitrile (16B)

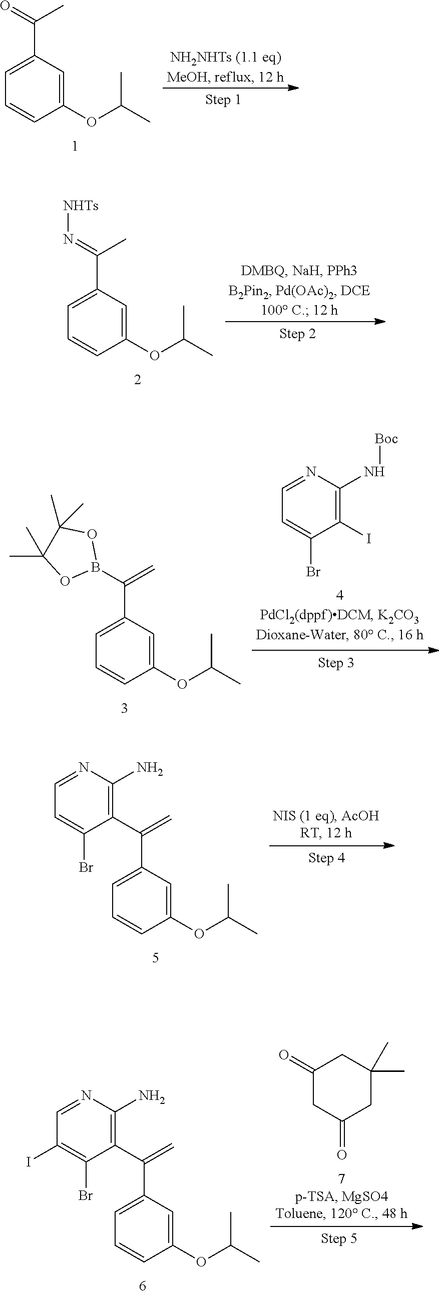

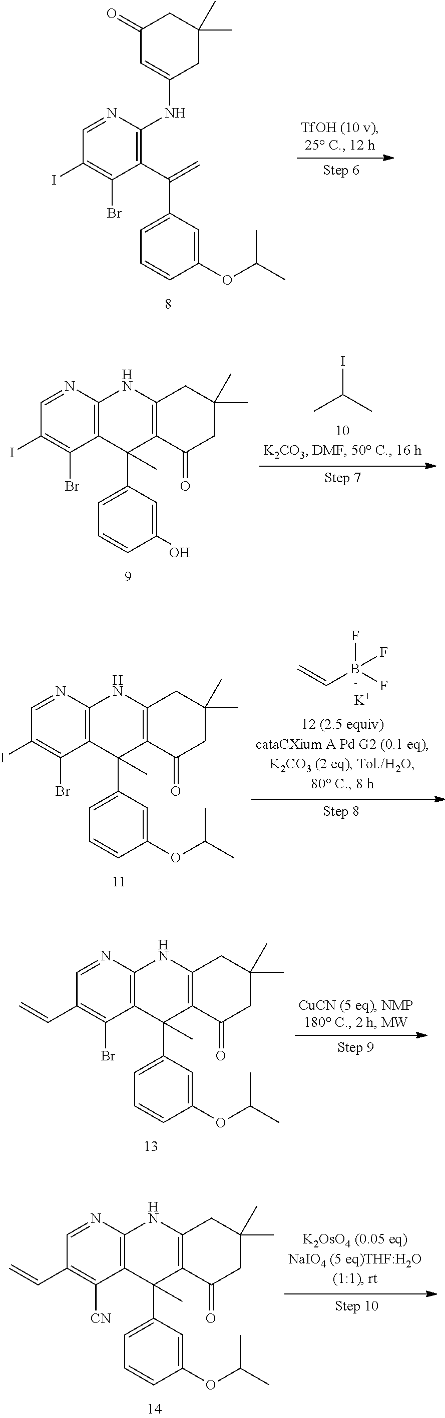

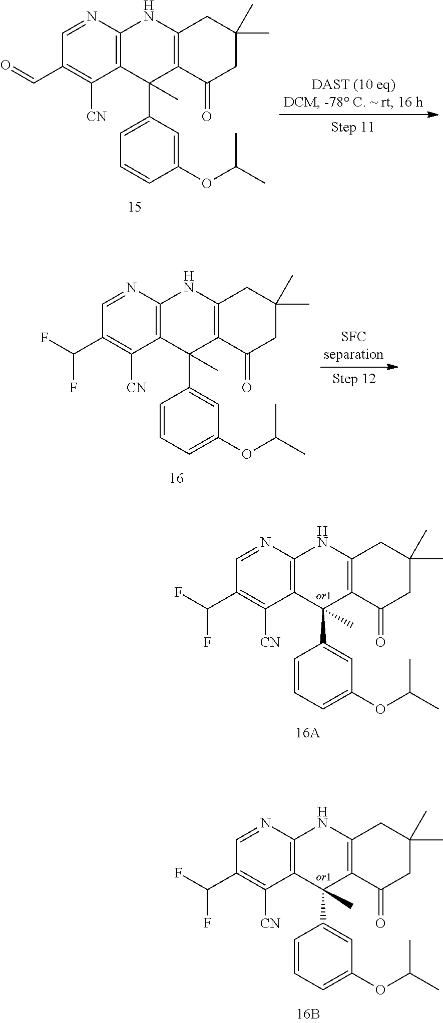 [SMILES: CC(C)Oc1cccc(C2(C)C3=C(CC(C)(C)CC3=O)Nc3ncc(C=O)c(C#N)c32)c1 ; CC(C)Oc1cccc(C2(C)C3=C(CC(C)(C)CC3=O)Nc3ncc(C(F)F)c(C#N)c32)c1 ; CC(C)Oc1cccc([C@]2(C)C3=C(CC(C)(C)CC3=O)Nc3ncc(C(F)F)c(C#N)c32)c1 ; CC(C)Oc1cccc([C@@]2(C)C3=C(CC(C)(C)CC3=O)Nc3ncc(C(F)F)c(C#N)c32)c1]

### Step 1: (E)-N′-(1-(3-isopropoxyphenyl)ethylidene)-4-methylbenzenesulfonohydrazide (2)

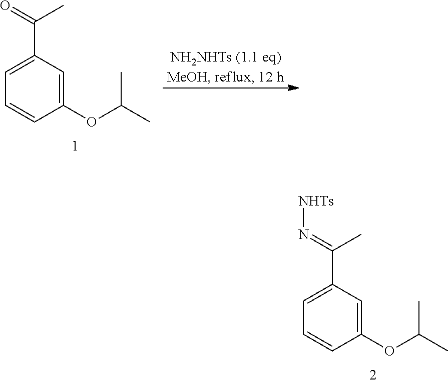

To the stirred solution of 1-(3-isopropoxyphenyl)ethenone (1) (16 g, 89.8 mmol, 1 eq) in MeOH (200 mL) was added p-Toluenesulfonyl hydrazide (18.4 g, 98.7 mmol, 1.1 eq) and the resulting mixture was allowed to reflux for 12 h under inert atmosphere. On completion, reaction mixture was concentrated under reduced pressure to get an off-white crude. Obtained crude was purified by CombiFlash column chromatography using 0-10% Ethyl acetate in hexane gradient to afford N—[(Z)-1-(3-isopropoxyphenyl)ethylideneamino]-4-methyl-benzenesulfonamide (2) (30 g, 86.6 mmol, 97% purity, 96% yield) as white solid.

LCMS: (ES⁺) m/z (M+H)⁺=347.0, Rt=3.62 min.

### Step 2: 2-(1-(3-isopropoxyphenyl)vinyl)-4,4,5,5-tetramethyl-1,3,2-dioxaborolane (3)

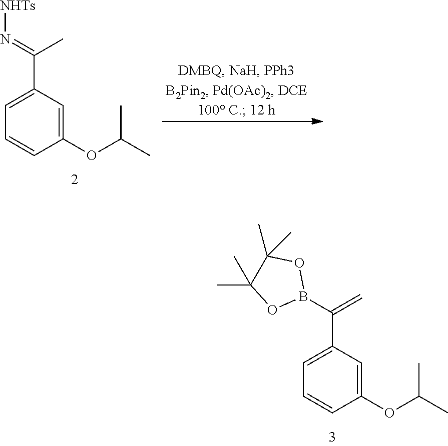

To a degassed stirred solution of N-[(E)-1-(3-isopropoxyphenyl)ethylideneamino]-4-methyl-benzenesulfonamide (2) (4 g, 11.5 mmol, 1 eq) and B₂Pin₂ (4.4 g, 17.3 mmol, 1.5 eq) in DCE (240 mL) were sequentially added PPh₃ (605 mg, 2.31 mmol, 0.2 eq), DMBQ (2.4 g, 17.3 mmol, 1.5 eq), NaH (60% in oil) (830 mg, 34.6 mmol, 3 eq) and Pd(OAc)₂ (259 mg, 1.15 mmol, 0.1 eq) under inert atmosphere. Resulting mixture was allowed to reflux for 12 h. On completion, reaction mixture was filtered through celite bed and washed with EtOAc. The filtrate was concentrated and was purified by CombiFlash chromatography using 0-10% EtOAc in hexane gradient to afford 2-[1-(3-isopropoxyphenyl)vinyl]-4,4,5,5-tetramethyl-1,3,2-dioxaborolane (3) (1.7 g, 5.54 mmol, 94% purity, 48% yield) as light yellow liquid.

LCMS: (ES⁺) m/z (M+H)⁺=289.2, Rt=2.30 min.

### Step 3: 4-bromo-3-(1-(3-isopropoxyphenyl)vinyl)pyridin-2-amine (5)

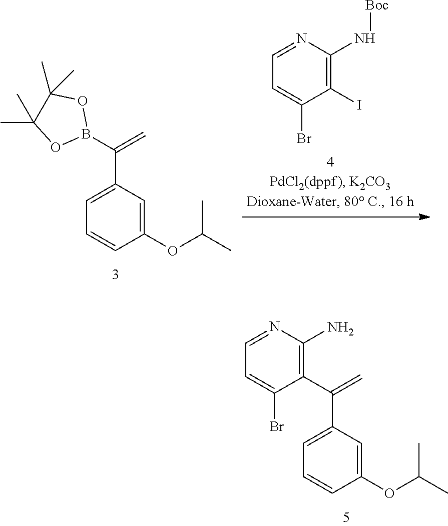 [SMILES: C=C(B1OC(C)(C)C(C)(C)O1)c1cccc(OC(C)C)c1 ; CC(C)(C)OC(=O)Nc1nccc(Br)c1I ; C=C(c1cccc(OC(C)C)c1)c1c(Br)ccnc1N]

To a degassed stirred solution of tert-butyl N-(4-bromo-3-iodo-2-pyridyl)carbamate (4) (6 g, 15 mmol, 1 eq), 2-[1-(3-isopropoxyphenyl)vinyl]-4,4,5,5-tetramethyl-1,3,2-dioxaborolane (3) (4.8 g, 16.5 mmol, 1.1 eq) and K₂CO₃ (5.2 g, 37.6 mmol, 2.5 eq) in 5:1 Dioxane-water mixture (66 mL) was added Pd(dppf)Cl₂ (1.2 g, 1.50 mmol, 0.1 eq) and the resulting mixture was allowed to stir at 80° C. for 16 h. Reaction mixture was poured into ethyl acetate, washed with water and brine. Obtained organic layer was dried over anhydrous Na₂SO₄, filtered and evaporated under vacuum to afford a brownish crude. Obtained crude was purified by CombiFlash column chromatography using 10-40% ethyl acetate in hexane gradient to afford 4-bromo-3-[1-(3-isopropoxyphenyl)vinyl]pyridin-2-amine (5) (4.2 g, 11 mmol, 87% purity, 73% yield).

LCMS: (ES⁺) m/z (M+H)⁺=332.8, Rt=3.66 min.

### Step 4: 4-bromo-5-iodo-3-(1-(3-isopropoxyphenyl)vinyl)pyridin-2-amine (6)

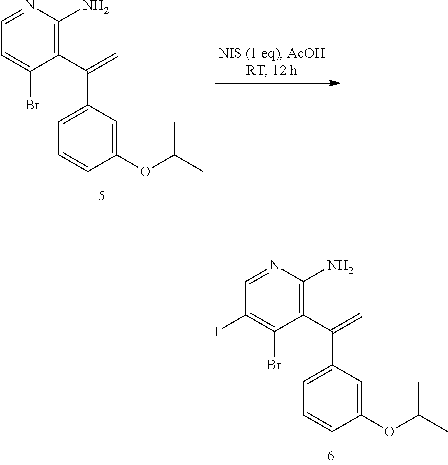 [SMILES: C=C(c1cccc(OC(C)C)c1)c1c(Br)ccnc1N ; C=C(c1cccc(OC(C)C)c1)c1c(N)ncc(I)c1Br]

To a stirred solution of 4-bromo-3-[I-(3-isopropoxyphenyl)vinyl]pyridin-2-amine (5) (3.8 g, 11.4 mmol, 1 eq) in AcOH (38 mL) was added NIS (2.6 g, 11.4 mmol, 1 eq) at 0° C. under inert atmosphere. Resulting mixture was allowed to stir at RT for 12 h. On completion, reaction mass was quenched with aqueous sodium bicarbonate solution and extracted with EtOAc (twice). Combined organic layer was sequentially washed with sodium thiosulphate solution, water and brine. The organic layer was then dried over Na₂SO₄, filtered and concentrated under vacuum to get a crude material which was purified by flash column chromatography using 5-30% EtOAc in hexane gradient to afford 4-bromo-5-iodo-3-[1-(3-isopropoxyphenyl)vinyl]pyridin-2-amine (6) (2.7 g, 4.76 mmol, 81% purity, 42% yield).

LCMS: (ES⁺) m/z (M+H)⁺=459.1, Rt=2.02 min.

### Step 5: 3-((4-bromo-5-iodo-3-(1-(3-isopropoxyphenyl)vinyl)pyridin-2-yl)amino)-5,5-dimethylcyclohex-2-en-1-one (8)

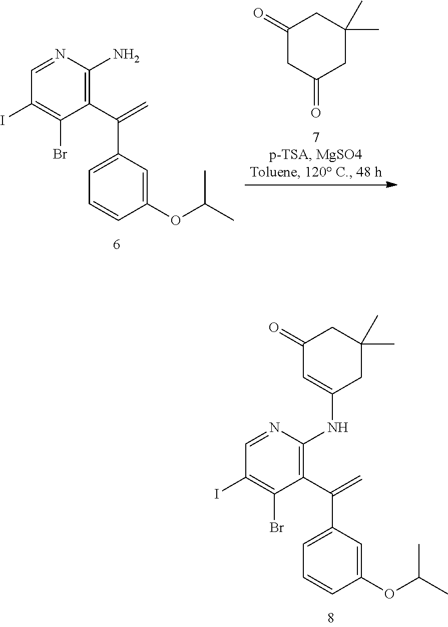

To a stirred solution of 4-bromo-5-iodo-3-[1-(3-isopropoxyphenyl)vinyl]pyridin-2-amine (6) (2 g, 4.36 mmol, 1 eq) and 5,5-dimethylcyclohexane-1,3-dione (7) (611 mg, 4.36 mmol, 1 eq) in toluene (25 mL) were added MgSO₄ (2091 mg, 17.4 mmol, 4.00 eq) and p-toluene sulfonic acid monohydrate (166 mg, 0.871 mmol, 0.2 eq) and the resulting mixture was allowed to stir at 120° C. for 48 h. On completion, reaction mixture was filtered and evaporated under reduced pressure to provide a brownish crude, which was purified by CombiFlash column chromatography using 20-60% EtOAc in hexane gradient to afford 3-[[4-bromo-5-iodo-3-[1-(3-isopropoxyphenyl)vinyl]-2-pyridyl]amino]-5,5-dimethyl-cyclohex-2-en-1-one (8) (1.4 g, 1.93 mmol, 80% purity, 44% yield) as yellow solid.

LCMS: (ES+) m/z (M+H)⁺=580.8, Rt=3.99 min.

### Step 6: 4-bromo-5-(3-hydroxyphenyl)-3-iodo-5,8,8-trimethyl-5,8,9,10-tetrahydrobenzo[b][1,8]naphthyridin-6(7H)-one (9)

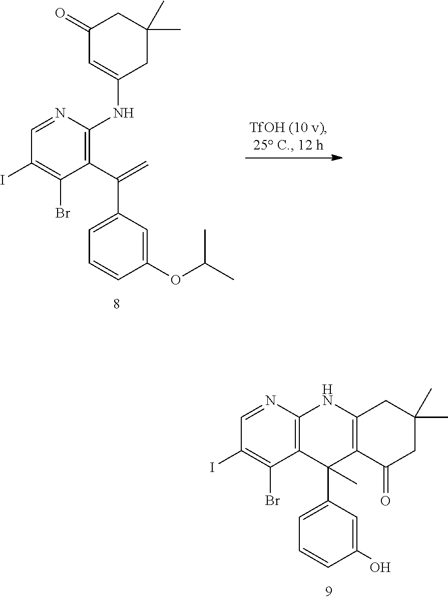 [SMILES: C=C(c1cccc(OC(C)C)c1)c1c(NC2=CC(=O)CC(C)(C)C2)ncc(I)c1Br ; CC1(C)CC(=O)C2=C(C1)Nc1ncc(I)c(Br)c1C2(C)c1cccc(O)c1]

TfOH (10 mL) was dropwise added to a RB flask containing 3-[[4-bromo-5-iodo-3-[1-(3-isopropoxyphenyl)vinyl]-2-pyridyl]amino]-5,5-dimethyl-cyclohex-2-en-1-one (8) (1 g, 1.72 mmol, 1 eq) at 0° C. under inert atmosphere. Resulting mixture in was allowed to stir at 25° C. for 12 h. On completion, reaction mixture was diluted with EtOAc and quenched with aqueous sodium bicarbonate solution. Obtained organic layer was sequentially washed with water and brine, dried over Na₂SO₄, filtered and concentrated to get a brownish crude. The crude was purified by CombiFlash column chromatography using 1-5% MeOH in DCM gradient to afford 4-bromo-5-(3-hydroxyphenyl)-3-iodo-5,8,8-trimethyl-9,10-dihydro-7H-benzo[b][1,8]naphthyridin-6-one (9) (330 mg, 0.379 mmol, 62% purity, 22% yield) as off white solid.

LCMS: (ES+) m/z (M+H)+=539.0, Rt=3.40 min.

### Step 7: 4-bromo-3-iodo-5-(3-isopropoxyphenyl)-5,8,8-trimethyl-5,8,9,10-tetrahydrobenzo[b][1,8]naphthyridin-6(7H)-one (11)

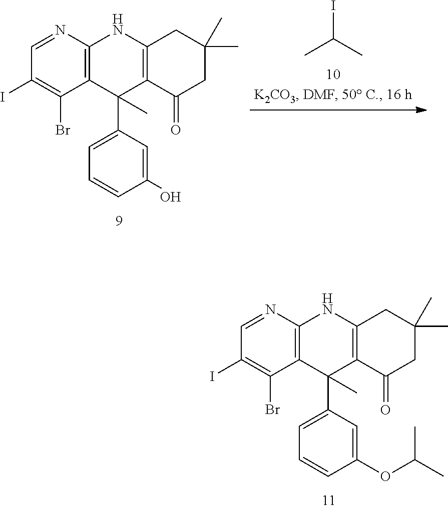

To a stirred solution of 4-bromo-5-(3-hydroxyphenyl)-3-iodo-5,8,8-trimethyl-9,10-dihydro-7H-benzo[b][1,8]naphthyridin-6-one (9) (360 mg, 0.67 mmol, 1 eq) in DMF (6 mL), taken in a sealed tube, were sequentially added potassium carbonate (192 mg, 1.38 mmol, 3 eq) and 2-iodopropane (10) (0.09 mL, 0.92 mmol, 2 eq) and the resulting mixture was allowed to stir at 50° C. for 16 h. On completion, the reaction mixture was diluted with EtOAc washed with water, brine, dried over Na₂SO₄, filtered and concentrated to afford a yellowish crude. Crude was purified by CombiFlash chromatography using 30-40% ethyl acetate in hexane gradient to afford 4-bromo-3-iodo-5-(3-isopropoxyphenyl)-5,8,8-trimethyl-9,10-dihydro-7H-benzo[b][1,8]naphthyridin-6-one (11) (190 mg, 0.3 mmol, 92% purity, 49% yield).

LCMS: (ES+) m/z (M+H)⁺=581.0, Rt=3.85 min.

### Step 8: 4-bromo-5-(3-isopropoxyphenyl)-5,8,8-trimethyl-3-vinyl-5,8,9,10-tetrahydrobenzo[b][1,8]naphthyridin-6(7H)-one (13)

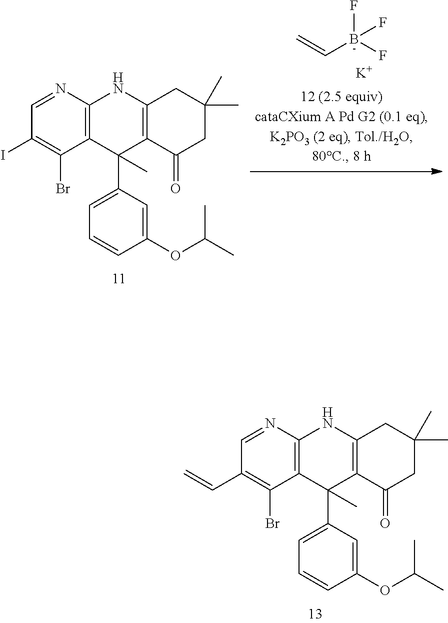

To a stirred degassed solution of 4-bromo-3-iodo-5-(3-isopropoxyphenyl)-5,8,8-trimethyl-9,10-dihydro-7H-benzo[b][1,8]naphthyridin-6-one (11) (210 mg, 0.36 mmol, 1 eq) in 4:1 toluene-water mixture (5 mL) were sequentially added potassium trifluoro(vinyl)boranuide (12) (121 mg, 0.9 mmol, 2.5 eq), K₂CO₃ (100 mg, 0.723 mmol, 2 eq) and cataCXium A Pd G2 (24 mg, 0.036 mmol, 0.1 eq). Resulting mixture was allowed to stir at 80° C. for 8 h. Reaction mixture was diluted with EtOAc, washed with water and brine. Organic layer was collected, dried over Na₂SO₄, filtered and concentrated under reduced pressure to afford a brownish crude, which was purified by CombiFlash column chromatography using 10-30% EtOAc in hexane gradient to afford 4-bromo-5-(3-isopropoxyphenyl)-5,8,8-trimethyl-3-vinyl-9,10-dihydro-7H-benzo[b][1,8]naphthyridin-6-one (13) (110 mg, 0.201 mmol, 88% purity, 56% yield).

LCMS: (ES+) m/z (M+H)+=481.2, Rt=2.23 min.

### Step 9: 5-(3-isopropoxyphenyl)-5,8,8-trimethyl-6-oxo-3-vinyl-5,6,7,8,9,10-hexahydrobenzo[b][1,8]naphthyridine-4-carbonitrile (14)

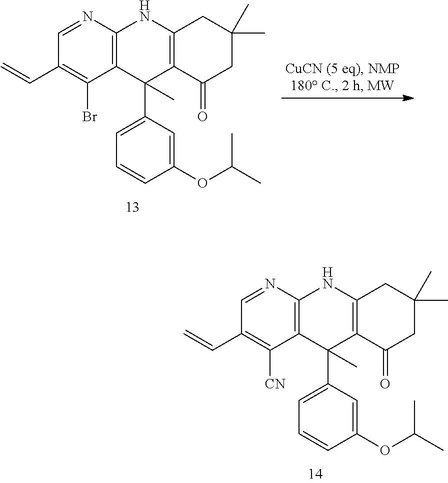 [SMILES: C=Cc1cnc2c(c1Br)C(C)(c1cccc(OC(C)C)c1)C1=C(CC(C)(C)CC1=O)N2 ; C=Cc1cnc2c(c1C#N)C(C)(c1cccc(OC(C)C)c1)C1=C(CC(C)(C)CC1=O)N2]

To a stirred solution of 4-bromo-5-(3-isopropoxyphenyl)-5,8,8-trimethyl-3-vinyl-9,10-dihydro-7H-benzo[b][1,8]naphthyridin-6-one (13) (110 mg, 0.228 mmol, 1.00 eq) in NMP (1.5 mL), taken in a microwave vial, was added CuCN (100 mg, 1.14 mmol, 5 eq) and the resulting mixture was allowed to stir at 180° C. under microwave irradiation for 2 h. On completion, the reaction mixture was diluted with EtOAc and washed with water and brine, dried over sodium sulfate, filtered and concentrated under reduced pressure to afford a yellowish crude. The crude material obtained was purified by CombiFlash column chromatography using 10-50% EtOAc in hexane gradient to afford 5-(3-isopropoxyphenyl)-5,8,8-trimethyl-6-oxo-3-vinyl-9,10-dihydro-7H-benzo[b][1,8]naphthyridine-4-carbonitrile (14) (45 mg, 0.1 mmol, 95% purity, 44% yield).

LCMS: (ES+) m/z (M+H)+=428.2, Rt=3.63 min.

### Step 10: 3-formyl-5-(3-isopropoxyphenyl)-5,8,8-trimethyl-6-oxo-5,6,7,8,9,10-hexahydrobenzo[b][1,8]naphthyridine-4-carbonitrile (15)

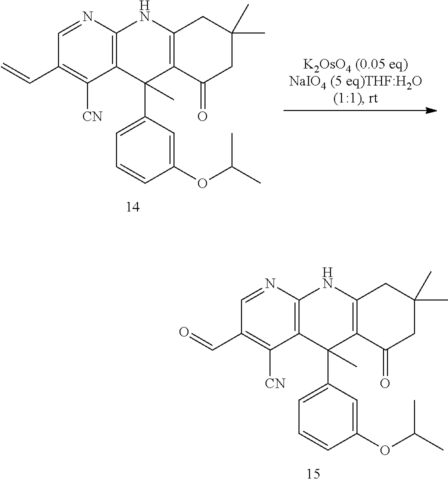 [SMILES: C=Cc1cnc2c(c1C#N)C(C)(c1cccc(OC(C)C)c1)C1=C(CC(C)(C)CC1=O)N2 ; CC(C)Oc1cccc(C2(C)C3=C(CC(C)(C)CC3=O)Nc3ncc(C=O)c(C#N)c32)c1]

To a stirred solution of 5-(3-isopropoxyphenyl)-5,8,8-trimethyl-6-oxo-3-vinyl-9,10-dihydro-7H-benzo[b][1,8]naphthyridine-4-carbonitrile (14) (45 mg, 0.105 mmol, 1 eq) in 1:1 THF—H₂O mixture (4 mL) was added potassium osmate (1.7 mg, 0.005 mmol, 0.05 eq) and Sodium periodate (113 mg, 0.53 mmol, 5 eq) and the resulting mixture was allowed to stir for 3 h at 25° C. Reaction mixture was diluted with water and extracted with EtOAc (twice). Combined organic layer was washed with brine, dried over Na₂SO₄, filtered and concentrated under reduced pressure to afford crude 3-formyl-5-(3-isopropoxyphenyl)-5,8,8-trimethyl-6-oxo-9,10-dihydro-7H-benzo[b][1,8]naphthyridine-4-carbonitrile (15) (45 mg, 0.072 mmol, 69% purity, 69% yield), which was used for next step without purification.

LCMS: (ES+) m/z (M+H)⁺=430.0, Rt=1.45 min.

### Step 11: 3-(difluoromethyl)-5-(3-isopropoxyphenyl)-5,8,8-trimethyl-6-oxo-5,6,7,8,9,10-hexahydrobenzo[b][1,8]naphthyridine-4-carbonitrile (16)

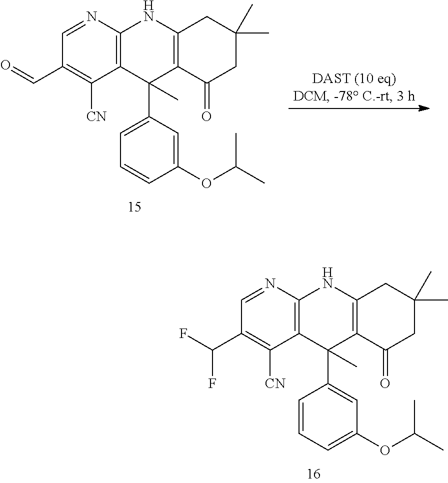 [SMILES: CC(C)Oc1cccc(C2(C)C3=C(CC(C)(C)CC3=O)Nc3ncc(C=O)c(C#N)c32)c1 ; CC(C)Oc1cccc(C2(C)C3=C(CC(C)(C)CC3=O)Nc3ncc(C(F)F)c(C#N)c32)c1]

DAST (0.18 mL, 1.40 mmol, 10 eq) was dropwise added to a stirred solution of 3-formyl-5-(3-isopropoxyphenyl)-5,8,8-trimethyl-6-oxo-9,10-dihydro-7H-benzo[b][1,8]naphthyridine-4-carbonitrile (15) (60 mg, 0.140 mmol, 1 eq) in DCM (3 mL), at −78° C. under inert atmosphere and the reaction was continued for 0.5 h under same temperature. Reaction mixture was then allowed to stir at 25° C. for additional 3 h. On completion, the reaction mixture was diluted with DCM, quenched with aqueous sodium bicarbonate solution, washed with water, brine, dried over Na₂SO₄, filtered and concentrated to afford a yellowish crude. Obtained crude was purified by CombiFlash column chromatography using 10-40% EtOAc in hexane gradient to afford 3-(difluoromethyl)-5-(3-isopropoxyphenyl)-5,8,8-trimethyl-6-oxo-9,10-dihydro-7H-benzo[b][1,8]naphthyridine-4-carbonitrile (16) (30 mg, 0.064 mmol, 97% purity, 46% yield) as yellow solid.

LCMS: (ES+) m/z (M+H)⁺=452.3, Rt=2.83 min.

### Step 12: (S)-3-(difluoromethyl)-5-(3-isopropoxyphenyl)-5,8,8-trimethyl-6-oxo-5,6,7,8,9,10-hexahydrobenzo[b][1,8]naphthyridine-4-carbonitrile (16A) and (R)-3-(difluoromethyl)-5-(3-isopropoxyphenyl)-5,8,8-trimethyl-6-oxo-5,6,7,8,9,10-hexahydrobenzo[b][1,8]naphthyridine-4-carbonitrile (16B)

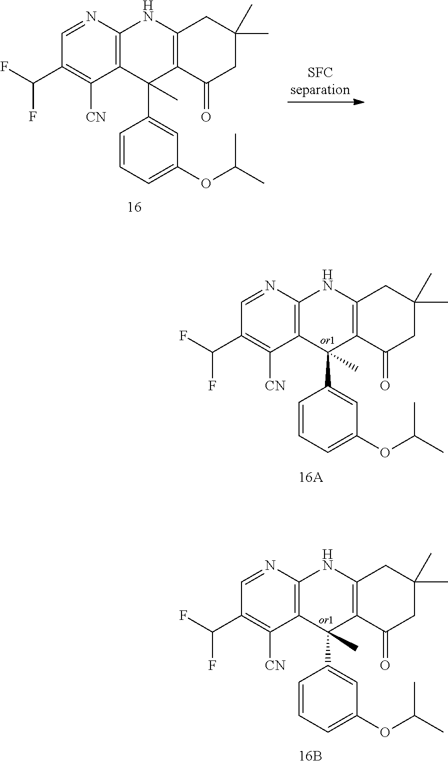 [SMILES: CC(C)Oc1cccc(C2(C)C3=C(CC(C)(C)CC3=O)Nc3ncc(C(F)F)c(C#N)c32)c1 ; CC(C)Oc1cccc([C@]2(C)C3=C(CC(C)(C)CC3=O)Nc3ncc(C(F)F)c(C#N)c32)c1 ; CC(C)Oc1cccc([C@@]2(C)C3=C(CC(C)(C)CC3=O)Nc3ncc(C(F)F)c(C#N)c32)c1]

Chiral separation of 3-(difluoromethyl)-5-(3-isopropoxyphenyl)-5,8,8-trimethyl-6-oxo-9,10-dihydro-7H-benzo[b][1,8]naphthyridine-4-carbonitrile (16) (30 mg, 0.064 mmol) was done by Waters PREP SFC 80 instrument equipped with Waters 2489 UV/Visible Detector by using (R,R)-Whelk-O-1 (21.1 mm×250 mm), 5μ Column operating at 35° C. temperature, maintaining flow rate of 70 ml/min, using 60% CO2 in super critical state & 40% of 100% methanol as Mobile phase. Run this isocratic mixture up to 10.0 minutes and also maintained the isobaric condition of 100 bar at 220 nm wavelength.

SFC fractions were collected and evaporated under reduced pressure to afford:

Peak 1: (S)-3-(difluoromethyl)-5-(3-isopropoxyphenyl)-5,8,8-trimethyl-6-oxo-5,6,7,8,9,10-hexahydrobenzo[b][1,8]naphthyridine-4-carbonitrile (16A) (8.7 mg, 0.0188 mmol, 97.7% purity, 28% yield).

UPLC (m/z)=452.2 [M+H] (RT=2.74)

¹H NMR (400 MHz, DMSO-d6) δ 10.46 (s, 1H), 8.43 (s, 1H), 7.09 (t, J=7.6 Hz, 1H), 7.02 (t, J=53.6 Hz, 1H), 6.92 (s, 1H), 6.78 (d, J=7.6 Hz, 1H), 6.66 (dd, J=8, 1.6 Hz, 1H), 4.57-4.47 (m, 1H), 2.45 (s, 2H), 2.14 (s, 3H), 2.03-1.88 (m, 2H), 1.22 (t, J=5.2 Hz, 6H), 0.99 (s, 3H), 0.91 (s, 3H).

[α]²⁵=269.009°, c=0.2524, ACN

Chiral purity (ee %): 100%

Peak 2: (R)-3-(difluoromethyl)-5-(3-isopropoxyphenyl)-5,8,8-trimethyl-6-oxo-5,6,7,8,9,10-hexahydrobenzo[b][1,8]naphthyridine-4-carbonitrile (16B) (7.3 mg, 0.0160 mmol, 98.22% purity, 24% yield).

UPLC (m/z)=452.2 [M+H] (RT=2.73)

¹H NMR (400 MHz, DMSO-d6) δ 10.46 (s, 1H), 8.43 (s, 1H), 7.09 (t, J=7.6 Hz, 1H), 7.02 (t, J=53.6 Hz, 1H), 6.92 (s, 1H), 6.78 (d, J=7.6 Hz, 1H), 6.66 (dd, J=8, 1.6 Hz, 1H), 4.57-4.47 (m, 1H), 2.45 (s, 2H), 2.14 (s, 3H), 2.03-1.88 (m, 2H), 1.22 (t, J=5.2 Hz, 6H), 0.99 (s, 3H), 0.91 (s, 3H).

[α]²⁵=−244.536°, c=0.2552, ACN.

Chiral purity (ee %): 100%.

### Example 3.12. (S)-11-(3-isopropoxyphenyl)-3,8,8,11-tetramethyl-3,6,7,8,9,11-hexahydro-10H-benzo[b]pyrazolo[4,3-f][1,8]naphthyridin-10-one (13A) & (R)-11-(3-isopropoxyphenyl)-3,8,8,11-tetramethyl-3,6,7,8,9,11-hexahydro-10H-benzo[b]pyrazolo[4,3-f][1,8]naphthyridin-10-one (13B)

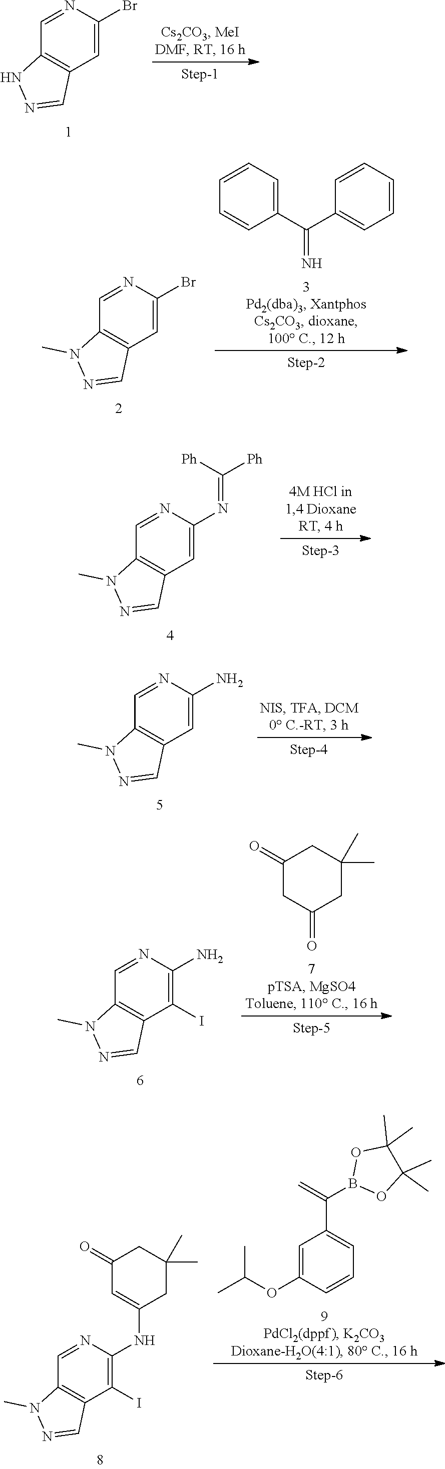

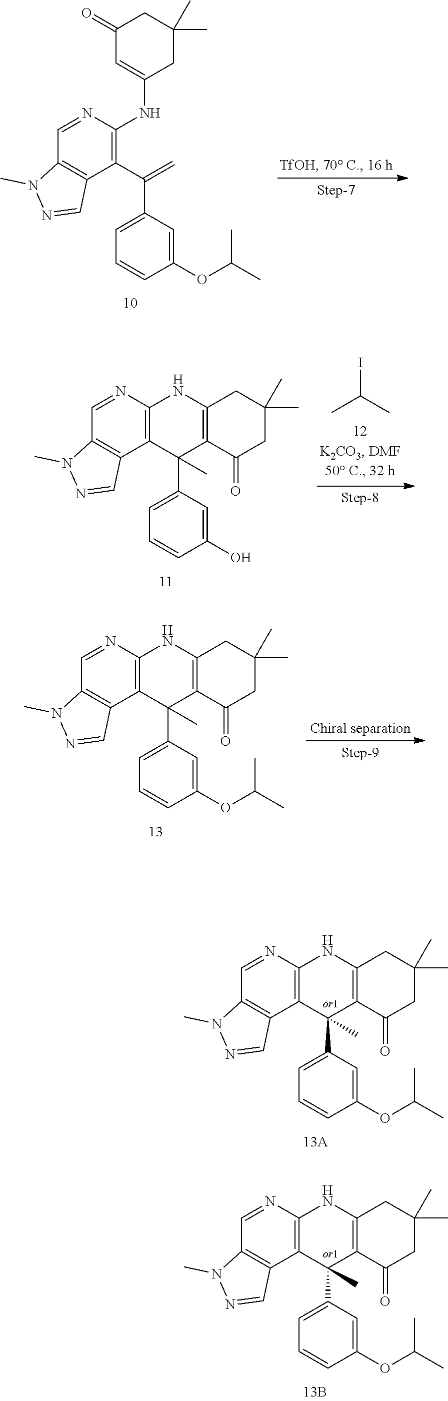

### Step 1: 5-bromo-1-methyl-pyrazolo[5,4-c]pyridine (2)

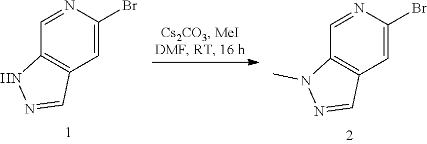

To a stirred solution of 5-bromo-1H-pyrazolo[5,4-c]pyridine (1) (15 g, 75.7 mmol, 1 eq) in DMF (20 mL) were added Cs₂CO₃ (37 g, 114 mmol, 1.5 eq) and MeI (5.7 mL, 90.9 mmol, 1.2 eq) under inert atmosphere. The resulting mixture was allowed to stir at RT for 16 h. On completion, the mixture was poured into ice water and extracted with ethyl acetate (twice). The combined organic layer was washed with brine, dried over anhydrous Na₂SO₄, filtered and evaporated under vacuum to afford a yellowish crude. Obtained crude was then purified by CombiFlash column chromatography using 5-20% ethyl acetate in hexane gradient to afford the desired isomers 5-bromo-1-methyl-pyrazolo[5,4-c]pyridine (2) (7.3 g, 34.4 mmol, 45% yield) as off white solid.

LCMS: Rt=2.70 min. (ES¹) m/z (M+H)⁺=211.9.

### Step 2: N-(1-methylpyrazolo[5,4-c]pyridin-5-yl)-1,1-diphenyl-methanimine (4)

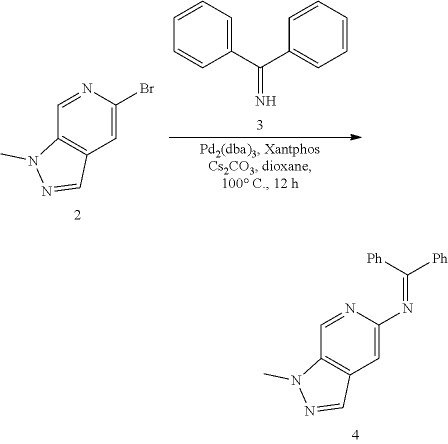 [SMILES: Cn1ncc2cc(Br)ncc21 ; N=C(c1ccccc1)c1ccccc1 ; Cn1ncc2cc(N=C(c3ccccc3)c3ccccc3)ncc21]

To a solution of 5-bromo-1-methyl-pyrazolo[5,4-c]pyridine (2) (10 g, 47.2 mmol, 1 eq), diphenylmethanimine (3) (17.1 g, 94.3 mmol, 2 eq) and Cs₂CO₃ (46.1 g, 141 mmol, 3 eq) in Dioxane (400 mL) argon was purged for 15 min. After that Xanthphos (10.9 g, 18.9 mmol, 0.4 eq) and Pd₂(dba)₃ (8.64 g, 9.43 mmol, 0.2 eq) were added and the reaction mixture and was allowed to stir at 100° C. for 12 h. On completion, the reaction mixture was cooled to RT, filtered through celite bed and washed with EtOAc. The filtrate was then washed with water, brine, dried over Na₂SO₄, filtered and evaporated under vacuum to afford a brownish crude. The crude residue was purified by CombiFlash column chromatography in EA/Hexane (0-20%) to afford the N-(1-methylpyrazolo[5,4-c]pyridin-5-yl)-1,1-diphenyl-methanimine (4) (8 g, 25.6 mmol, 54% yield).

LCMS: Rt=3.34 min. (ES⁺) m/z (M+H)⁺=312.8.

### Step 3: 1-methylpyrazolo[5,4-c]pyridin-5-amine (5)

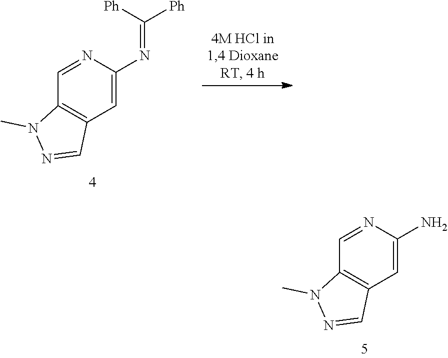 [SMILES: Cn1ncc2cc(N=C(c3ccccc3)c3ccccc3)ncc21 ; Cn1ncc2cc(N)ncc21]

To a stirred solution of N-(1-methylpyrazolo[5,4-c]pyridin-5-yl)-1,1-diphenyl-methanimine (4) (8 g, 25.6 mmol, 1 eq) in Dioxane (10 mL) was added 4N HCl in Dioxane (32 mL, 128 mmol, 5 eq) dropwise under inert atmosphere at 0° C. Resulting mixture was then allowed to stir for 4 h at RT. On completion, volatiles were evaporated under reduced pressure, neutralized by aq K2CO3 solution, extracted multiple times with EtOAc. Combined organic part was then dried over sodium sulfate, filtered and evaporated under reduced pressure to afford 1-methylpyrazolo[5,4-c]pyridin-5-amine (5) (3.0 g, 20.2 mmol, 79% yield) (contaminated with benzophenone residue) as brown solid which used as such for the next step without further purification.

LCMS: Rt=1.05 min, (ES⁺) m/z (M+H)⁺=149.1.

### Step 4: 4-iodo-1-methyl-pyrazolo[5,4-c]pyridin-5-amine. (6)

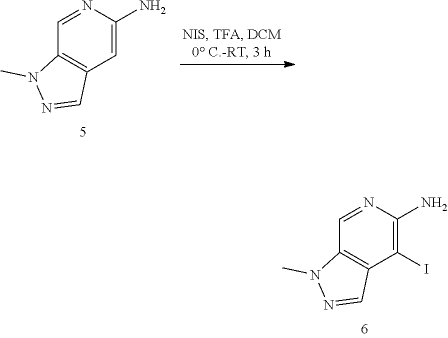

To a stirred solution of 1-methylpyrazolo[5,4-c]pyridin-5-amine (5) (3 g, 20.2 mmol, 1 eq) and TFA (1.6 mL, 20.2 mmol, 1 eq) in DCM (120 mL) was added NIS (4.6 g, 20.2 mmol, 1 eq) portion-wise at 0° C. under inert atmosphere. Then the reaction mixture was allowed to stir at RT for 3 h. On completion, the reaction mixture was quenched with aq. NaHCO₃ solution and extracted with ethyl acetate (twice). The organic part was then washed with water and brine, dried over sodium sulfate, filtered and concentrated under vacuo to afford the crude of 4-iodo-1-methyl-pyrazolo[5,4-c]pyridin-5-amine (6) (3.5 g, 12.8 mmol, 63% yield), which used as such for the next step without further purification.

LCMS: Rt=2.55 min, (ES⁺) m/z (M+H)⁺=274.9.

### Step 5: 3-[(4-iodo-1-methyl-pyrazolo[5,4-c]pyridin-5-yl)amino]-5,5-dimethyl-cyclohex-2-en-1-one (8)

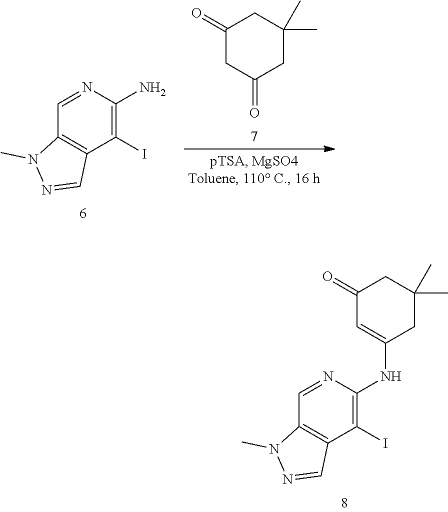

To a stirred solution of 4-iodo-1-methyl-pyrazolo[5,4-c]pyridin-5-amine (6) (3.5 g, 12.8 mmol, 1 eq) and 5,5-dimethylcyclohexane-1,3-dione (7) (2.15 g, 15.3 mmol, 1.2 eq) in toluene (30 mL) were added pTSA (485 mg, 2.55 mmol, 0.2 eq) and MgSO₄ (12.3 g, 102 mmol, 8 eq) under inert atmosphere. Then the reaction mixture was allowed to stir at 110° C. for 16 h. On completion, the reaction mass was cooled to RT, diluted with EtOAc, filtered and the obtained filtrate was washed with water and brine. The organic layer was then dried over sodium sulfate, filtered and concentrated under reduced pressure to afford the crude. Crude residue was purified by CombiFlash column chromatography using 5-40% EtOAc-DCM gradient to afford 3-[(4-iodo-1-methyl-pyrazolo[5,4-c]pyridin-5-yl)amino]-5,5-dimethyl-cyclohex-2-en-1-one (8) (1.6 g, 4.04 mmol, 32% yield) as yellow solid.

LCMS: Rt=2.80 min, (ES⁺) m/z (M+H)⁺=397.0.

### Step 6: 3-[[4-[1-(3-isopropoxyphenyl)vinyl]-1-methyl-pyrazolo[5,4-c]pyridin-5-yl]amino]-5,5-dimethyl-cyclohex-2-en-1-one. (10)

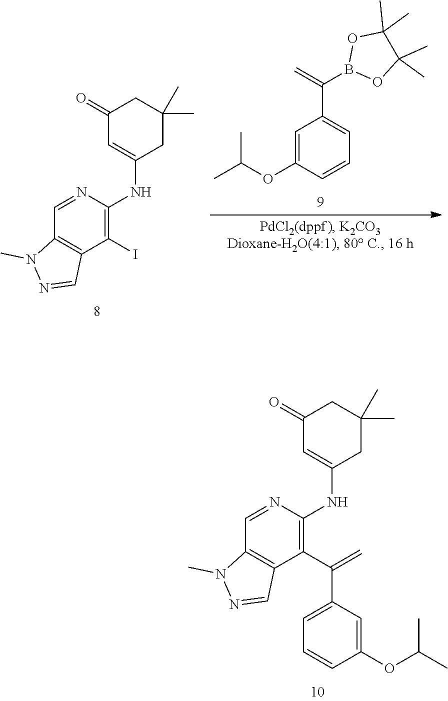

A solution of 3-[(4-iodo-1-methyl-pyrazolo[5,4-c]pyridin-5-yl)amino]-5,5-dimethyl-cyclohex-2-en-1-one (8) (1.6 g, 4.04 mmol, 1 eq), 2-[1-(3-isopropoxyphenyl)vinyl]-4,4,5,5-tetramethyl-1,3,2-dioxaborolane (9) (1.3 g, 4.44 mmol, 1.1 eq) and K₂CO₃ (1.4 g, 10.1 mmol, 2.5 eq) in 4:1 dioxane:H₂O mixture (15 mL) was purged argon for 15 min. After that Pd(dppf)Cl₂ (330 mg, 0.404 mmol, 0.1 eq) was added and the reaction mixture was allowed to stir at 80° C. for 16 h under inert atmosphere. On completion, the reaction mixture was cooled to RT and was filtered through celite bed and washed with ethyl acetate (twice). Combined filtrate was then washed with water, brine, dried over anhydrous Na₂SO₄ and evaporated under vacuo to afford a brownish crude. The crude was purified by CombiFlash column chromatography using 10-40% ethyl acetate-hexane gradient to afford 3-[[4-[1-(3-isopropoxyphenyl)vinyl]-1-methyl-pyrazolo[5,4-c]pyridin-5-yl]amino]-5,5-dimethyl-cyclohex-2-en-1-one (10) (500 mg, 1.08 mmol, 93% purity, 27% yield) as light yellow solid.

LCMS: Rt=3.22 min, (ES⁺) m/z (M+H)⁺=431.0.

### Step 7: 11-(3-hydroxyphenyl)-3,8,8,11-tetramethyl-3,6,7,8,9,11-hexahydro-10H-benzo[b]pyrazolo[4,3-f][1,8]naphthyridin-10-one (11)

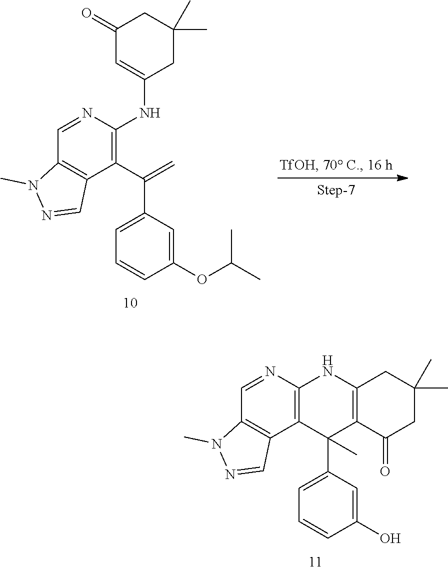

TfOH (10 mL) was added dropwise to a RB flask containing 3-[[4-[1-(3-isopropoxyphenyl) vinyl]-1-methyl-pyrazolo[5,4-c]pyridin-5-yl]amino]-5,5-dimethyl-cyclohex-2-en-1-one (10) (500 mg, 1.16 mmol, 1 eq) under inert atmosphere at 0° C. The resulting mixture was stirred at 60° C. for 16 h. On completion, the reaction mixture was neutralized by ice-cooled NaHCO₃ solution and extracted with EtOAc. The organic layer was then dried over sodium sulfate, filtered and concentrated under reduced pressure to furnish crude 11-(3-hydroxyphenyl)-3,8,8,11-tetramethyl-3,6,7,8,9,11-hexahydro-10H-benzo[b]pyrazolo[4,3-f][1,8]naphthyridin-10-one (11) (215 mg, 0.42 mmol, 76% purity, 36% yield) as yellowish gummy solid, which used as such for the next step without further purification.

LCMS: Rt=1.59 min, (ES⁺) m/z (M+H)⁺=389.3.

### Step 8: 11-(3-isopropoxyphenyl)-3,8,8,11-tetramethyl-3,6,7,8,9,11-hexahydro-10H-benzo[b]pyrazolo[4,3-f][1,8]naphthyridin-10-one (13)

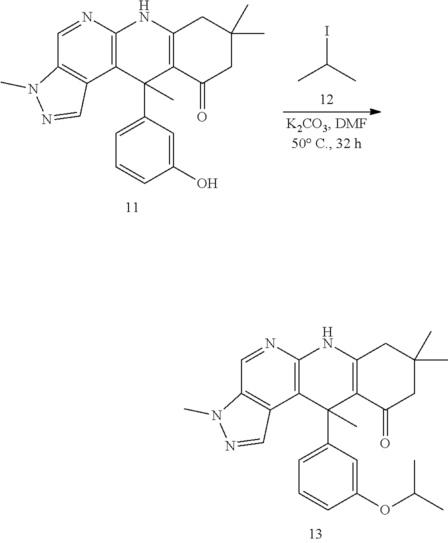

To a stirred solution of 11-(3-hydroxyphenyl)-3,8,8,11-tetramethyl-3,6,7,8,9,11-hexahydro-10H-benzo[b]pyrazolo[4,3-f][1,8]naphthyridin-10-one (11) (215 mg, 0.553 mmol, 1 eq) and K₂CO₃ (231 mg, 1.66 mmol, 3 eq) in DMF (5 mL), taken in a sealed tube, was added 2-iodopropane (12) (0.11 mL, 1.11 mmol, 2 eq) and then the reaction mixture was allowed to stir for 32 h at 50° C. On completion, ice-water was added to the reaction mixture, extracted with ethyl acetate thrice. The combined organic part was collected, washed with brine, dried over Na₂SO₄, filtered off and concentrated to afford a yellowish crude. The crude was purified by CombiFlash column chromatography using 30-40% ethyl acetate in hexane gradient to afford 11-(3-isopropoxyphenyl)-3,8,8,11-tetramethyl-3,6,7,8,9,11-hexahydro-10H-benzo[b]pyrazolo[4,3-f][1,8]naphthyridin-10-one (13) (105 mg, 0.234 mmol, 96% purity, 42% yield) as off white solid.

LCMS: Rt=2.65 min, (ES⁺) m/z (M+H)⁺=431.3.

### Step 9: Chiral Separation

### (S)-11-(3-isopropoxyphenyl)-3,8,8,11-tetramethyl-3,6,7,8,9,11-hexahydro-10H-benzo[b]pyrazolo[4,3-f][1,8]naphthyridin-10-one (13A) & (R)-11-(3-isopropoxyphenyl)-3,8,8,11-tetramethyl-3,6,7,8,9,11-hexahydro-10H-benzo[b]pyrazolo[4,3-f][1,8]naphthyridin-10-one (13B)

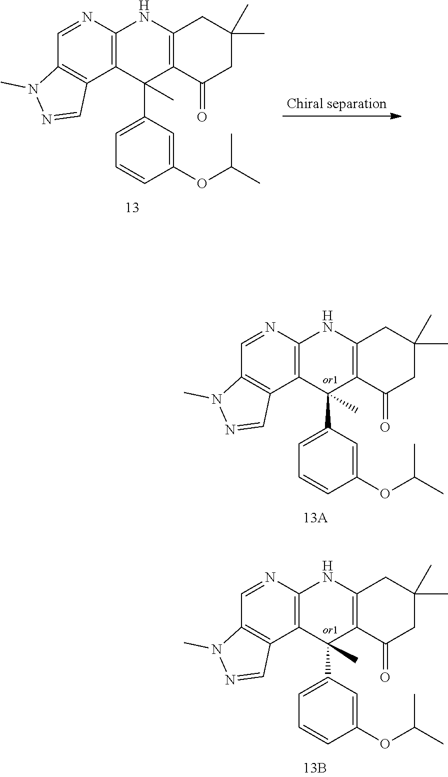

Chiral Prep SFC separation of 11-(3-isopropoxyphenyl)-3,8,8,11-tetramethyl-3,6,7,8,9,11-hexahydro-10H-benzo[b]pyrazolo[4,3-f][1,8]naphthyridin-10-one (13) (105 mg, 0.244 mmol) was done on PIC SOLUTIONS-175 instrument equipped with Knauer 40D Detector by using Chiralpak IG (30.0 mm×250 mm), 5μ Column operating at 35° C. temperature, maintaining flow rate of 100 ml/min using 85% CO₂ in super critical state & 15% [100% MeOH] as Mobile phase, Run this isocratic mixture up to 16.0 minutes and also maintained the isobaric condition of 100 bar at 220 nm wavelength.

SFC fractions were collected and evaporated under reduced pressure to afford:

Peak 1: (S)-11-(3-isopropoxyphenyl)-3,8,8,11-tetramethyl-3,6,7,8,9,11-hexahydro-OH-benzo[b]pyrazolo[4,3-f][1,8]naphthyridin-10-one (13A) (26 mg, 0.0577 mmol, 96.78% purity, 24% yield) as off white solid.

UPLC: Rt=2.36 min, (ES⁺) m/z (M+H)⁺=431.3.

1H NMR (400 MHz, DMSO-d6) δ 9.68 (s, 1H), 8.66 (s, 1H), 7.35 (s, 1H), 7.09-6.99 (m, 3H), 6.58 (d, J=8.0 Hz, 1H), 4.53-4.47 (m, 1H), 3.97 (s, 3H), 2.45 (s, 2H), 2.09 (s, 3H), 2.03-1.87 (m, 2H), 1.21-1.14 (m, 6H), 1.00 (s, 3H), 0.97 (s, 3H).

[α]²⁵=+89.656, c=0.2621, MeOH.

Chiral purity (ee %): 100

Peak 2: (R)-11-(3-isopropoxyphenyl)-3,8,8,11-tetramethyl-3,6,7,8,9,11-hexahydro-10H-benzo[b]pyrazolo[4,3-f][1,8]naphthyridin-10-one (13B) (22 mg, 0.0506 mmol, 97.36% purity, 21% yield) as off white solid.

UPLC: Rt=2.37 min, (ES⁺) m/z (M+H)⁺=431.3.

1H NMR (400 MHz, DMSO-d6) δ 9.68 (s, 1H), 8.66 (s, 1H), 7.35 (s, 1H), 7.09-6.99 (m, 3H), 6.58 (d, J=8.0 Hz, 1H), 4.53-4.47 (m, 1H), 3.97 (s, 3H), 2.45 (s, 2H), 2.09 (s, 3H), 2.03-1.87 (m, 2H), 1.21-1.14 (m, 6H), 1.00 (s, 3H), 0.97 (s, 3H).

[α]²⁵=−94.405, c=0.2532, MeOH.

Chiral purity (ee %): 100

### Example 3.13. (S)-5-(3-(difluoromethyl)phenyl)-3-fluoro-5,8,8-trimethyl-5,8,9,10-tetrahydrobenzo[b][1,8]naphthyridin-6(7H)-one (7A) & (R)-5-(3-(difluoromethyl)phenyl)-3-fluoro-5,8,8-trimethyl-5,8,9,10-tetrahydrobenzo[b][1,8]naphthyridin-6(7H)-one (7B)

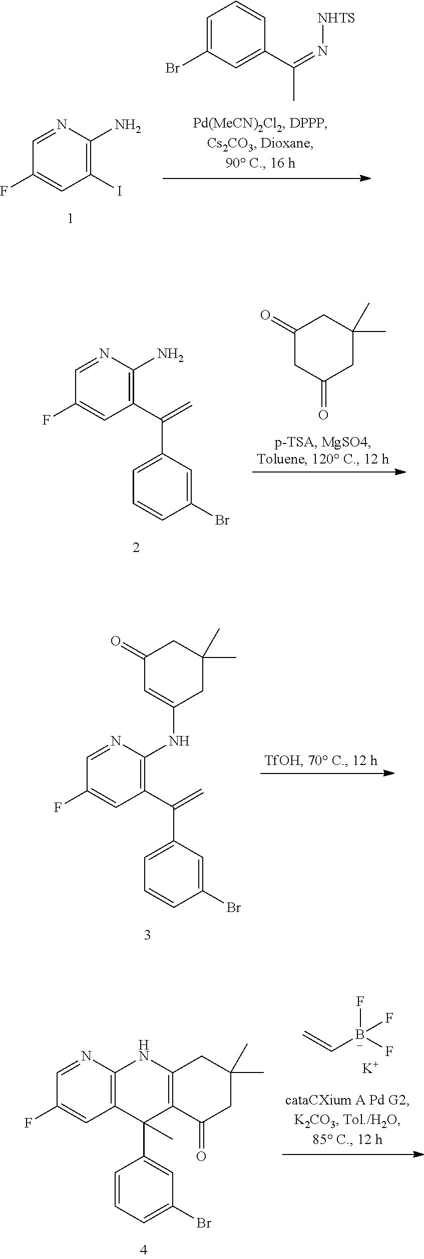

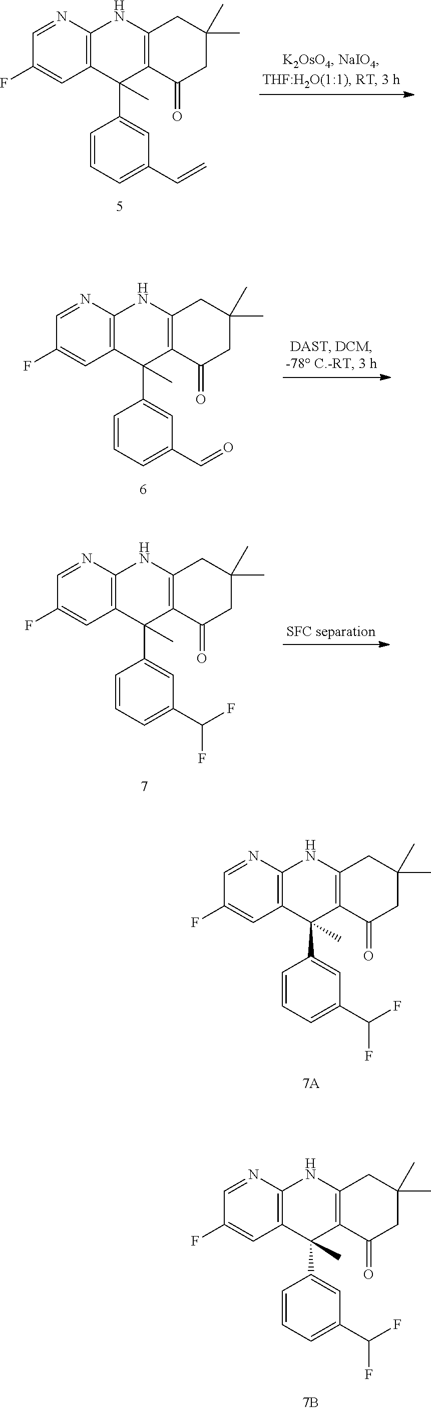 [SMILES: C=Cc1cccc(C2(C)C3=C(CC(C)(C)CC3=O)Nc3ncc(F)cc32)c1 ; CC1(C)CC(=O)C2=C(C1)Nc1ncc(F)cc1C2(C)c1cccc(C=O)c1 ; CC1(C)CC(=O)C2=C(C1)Nc1ncc(F)cc1C2(C)c1cccc(C(F)F)c1 ; CC1(C)CC(=O)C2=C(C1)Nc1ncc(F)cc1[C@]2(C)c1cccc(C(F)F)c1 ; CC1(C)CC(=O)C2=C(C1)Nc1ncc(F)cc1[C@@]2(C)c1cccc(C(F)F)c1]

### Step 1: 341-(3-bromophenyl)vinyl)-5-fluoropyridin-2-amine. (2)

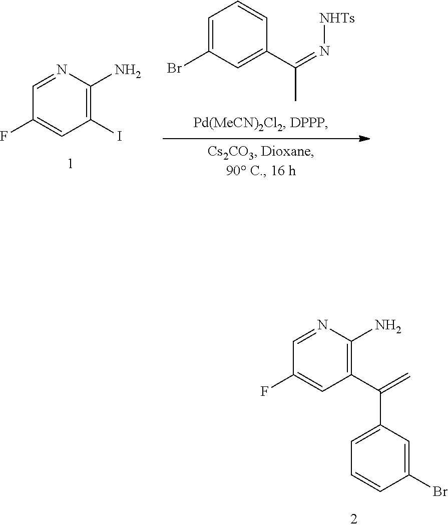

The solution of (Z)—N′-(1-(3-bromophenyl)ethylidene)-4-methylbenzenesulfonohydrazide (1) (10.80 g, 29.4 mmol, 1.4 eq), DPPP (2.60 g, 6.3 mmol, 0.3 eq) and cesium carbonate (17.11 g, 52.5 mmol, 2.5 eq) in Dioxane (300 mL) was bubbled with argon for 10 min, then Bis(acetonitrile)dichloropalladium(II) (0.82 g, 3.15 mmol, 0.15 eq) and 5-fluoro-3-iodopyridin-2-amine (5.0 g, 21.0 mmol, 1.0 eq) were added. The reaction mixture was allowed to stir at 90° C. for 16 h. Reaction mixture was filtered through celite pad and washed with ethyl acetate. The filtrate was then washed with water and brine, dried over sodium sulphate and concentrated under reduced pressure to afford a brownish crude. Obtained crude was purified by CombiFlash column chromatography using 5-9% Ethyl-hexane gradient to afford 3-(1-(3-bromophenyl)viny)-5-fluoropyridin-2-amine (2) (1.0 g, 3.41 mmol, 16% yield) yellow solid.

LCMS: Rt=3.54 min, (ES⁺) m/z (M+H)⁺=293.1.

### Step 2: 3-((3-(1-(3-bromophenyl)vinyl)-5-fluoropyridin-2-yl)amino)-5,5-dimethylcyclohex-2-en-1-one. (3)

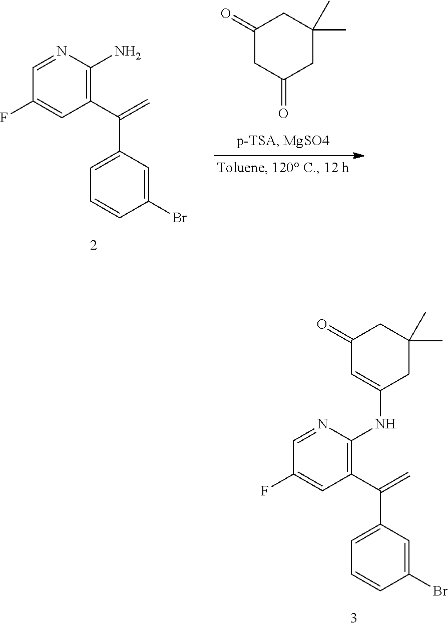

To the stirred solution of 3-(1-(3-bromophenyl)vinyl)-5-fluoropyridin-2-amine (2) (1.0 g, 3.41 mmol, 1.0 eq) in Toluene (12 mL) were added 5,5-dimethylcyclohexane-1,3-dione (717 mg, 5.12 mmol, 1.5 eq), MgSO4 (4.1 mg, 34.1 mmol, 10.0 eq) and pTSA (65 mg, 0.341 mmol, 0.100 eq) sequentially under inert atmosphere at RT. Resulting mixture was allowed to stir at 120° C. for 12 h. Reaction mixture was filtered through a celite pad and washed with ethyl acetate. Obtained filtrate was evaporated under vacuo to afford a brownish crude, which was purified by combiflash chromatography using 10-20% ethyl acetate in hexane gradient to furnish 3-((3-(1-(3-bromophenyl)vinyl)-5-fluoropyridin-2-yl)amino)-5,5-dimethylcyclohex-2-en-1-one (3) (650 mg, 1.49 mmol, 95% purity, 44% yield) as yellow solid.

LCMS: Rt=3.48 min, (ES⁺) m/z (M+H)⁺=414.7.

### Step 3: 5-(3-bromophenyl)-3-fluoro-5,8,8-trimethyl-5,8,9,10-tetrahydrobenzo[b][1,8] naphthyridin-6(7H)-one. (4)

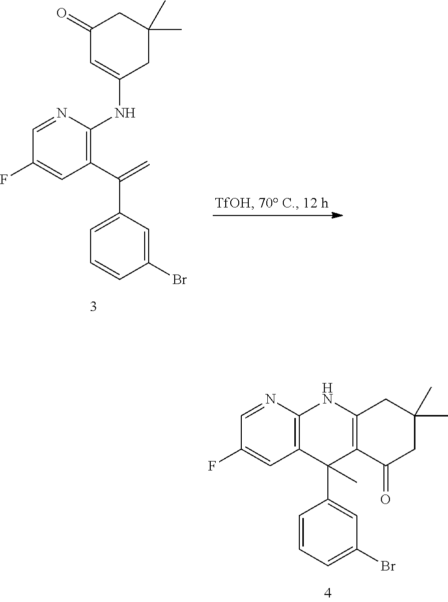 [SMILES: C=C(c1cccc(Br)c1)c1cc(F)cnc1NC1=CC(=O)CC(C)(C)C1 ; CC1(C)CC(=O)C2=C(C1)Nc1ncc(F)cc1C2(C)c1cccc(Br)c1]

TfOH (6 mL) was added dropwise to a RB flask containing 3-((3-(1-(3-bromophenyl)vinyl)-5-fluoropyridin-2-yl)amino)-5,5-dimethylcyclohex-2-en-1-one (3) (650 mg, 1.57 mmol, 1.0 eq) at ice cold condition under N₂ atmosphere and heated the reaction mixture at 70° C. for 12 h. Reaction mixture was quenched with aq. NaHCO₃ solution and extracted by ethyl acetate (twice). Combined organic layer was washed with brine, dried over anhydrous Na₂SO₄, filtered and concentrated under vacuo to afford a brownish crude, which purified by silica gel combiflash chromatography using 13-15% ethyl acetate in hexane gradient to afford 5-(3-bromophenyl)-3-fluoro-5,8,8-trimethyl-5,8,9,10-tetrahydrobenzo[b][1,8]naphthyridin-6(7H)-one (4) (410 mg, 0.987 mmol, 100% purity, 63% yield) as light brownish solid.

LCMS: Rt=3.53 min, (ES⁺) m/z (M+H)⁺=414.9.

### Step 4: 3-fluoro-5,8,8-trimethyl-5-(3-vinylphenyl)-5,8,9,10-tetrahydrobenzo[b][1,8] naphthyridine-6(7H)-one. (5)

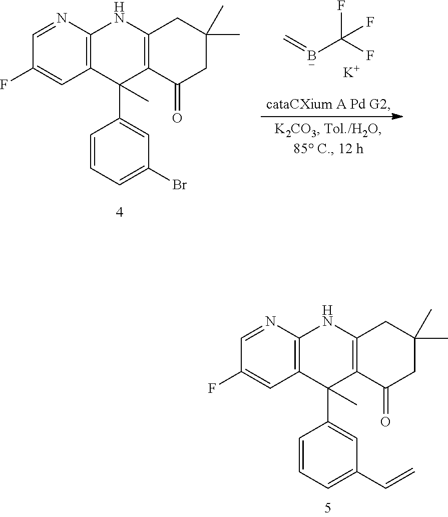

To the degassed solution of 5-(3-bromophenyl)-3-fluoro-5,8,8-trimethyl-5,8,9,10-tetrahydrobenzo[b][1,8]naphthyridin-6(7H)-one (4) (410 mg, 0.987 mmol, 1.0 eq) in Toluene; Water (4:1) (15 mL), potassium trifluoro(vinyl)boranuide (198 mg, 1.48 mmol, 1.5 eq), K₂CO₃ (273 mg, 1.97 mmol, 2.0 eq) and cataCXium A Pd G2 (66 mg, 0.1 mmol, 0.1 eq) were added sequentially. The resultant reaction mixture was stirred at 85° C. for 12 h. After cooled to RT, reaction mixture was diluted with EtOAC and washed sequentially with water and brine. Organic part was collected, dried over Na₂SO₄, filtered and concentrated. Residue was purified by silica gel combiflash column chromatography using 15-25% EtOAc in hexane gradient to furnish 3-fluoro-5,8,8-trimethyl-5-(3-vinylphenyl)-5,8,9,10-tetrahydrobenzo[b][1,8]naphthyridin-6(7H)-one (5) (265 mg, 0.695 mmol, 95% purity, 70% yield) as light yellow solid.

LCMS: Rt=3.53 min, (ES⁺) m/z (M+H)⁺=363.2.

### Step 5: 3-(3-fluoro-5,8,8-trimethyl-6-oxo-5,6,7,8,9,10-hexahydrobenzo[b][1,8]naphthyridin-5-yl)benzaldehyde (6)

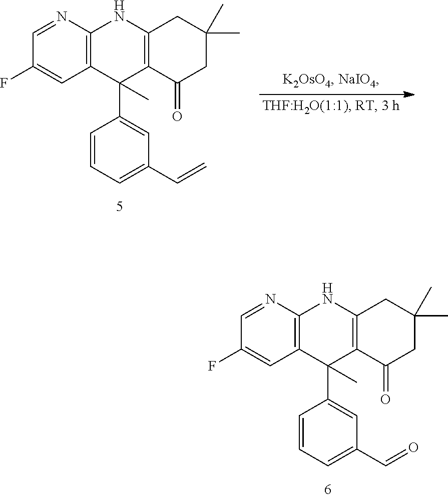 [SMILES: C=Cc1cccc(C2(C)C3=C(CC(C)(C)CC3=O)Nc3ncc(F)cc32)c1 ; CC1(C)CC(=O)C2=C(C1)Nc1ncc(F)cc1C2(C)c1cccc(C=O)c1]

To a stirred solution of 3-fluoro-5,8,8-trimethyl-5-(3-vinylphenyl)-5,8,9,10-tetrahydrobenzo[b][1,8]naphthyridin-6(7H)-one (5) (265 mg, 0.731 mmol, 1.0 eq) in 1:1 THF-H₂O mixture (8 mL) was added potassium osmate (12 mg, 0.037 mmol, 0.05 eq) and sodium periodate (782 mg, 3.66 mmol, 5 eq) and allowed to stir for 3 h at RT. Then reaction mixture was diluted with EtOAc and washed sequentially with water and brine, dried over Na₂SO₄, filtered off and concentrated under reduced pressure to get a reddish crude, which was triturated using ether and pentane to afford crude 3-(3-fluoro-5,8,8-trimethyl-6-oxo-5,6,7,8,9,10-hexahydrobenzo[b][1,8]naphthyridin-5-yl)benzaldehyde (6) (255 mg, 0.658 mmol, 94% purity, 90% yield) as brownish solid. This compound was used as such for next step without further purification.

LCMS: Rt=3.18 min, (ES⁺) m/z (M+H)⁺=365.2.

### Step 6: 5-(3-(difluoromethyl)phenyl)-3-fluoro-5,8,8-trimethyl-5,8,9,10-tetrahydrobenzo[b][1,8]naphthyridin-6(7H)-one (7)

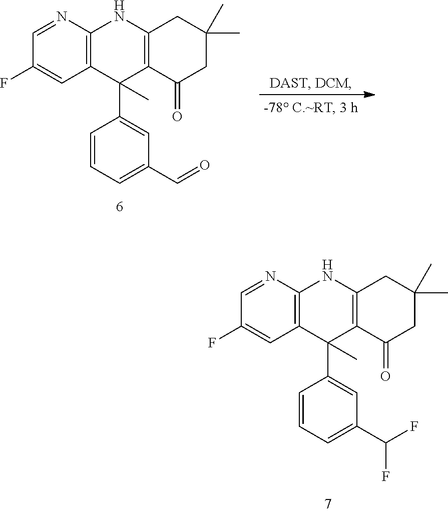 [SMILES: CC1(C)CC(=O)C2=C(C1)Nc1ncc(F)cc1C2(C)c1cccc(C=O)c1 ; CC1(C)CC(=O)C2=C(C1)Nc1ncc(F)cc1C2(C)c1cccc(C(F)F)c1]

To the solution of 3-(3-fluoro-5,8,8-trimethyl-6-oxo-5,6,7,8,9,10-hexahydrobenzo[b][1,8]naphthyridin-5-yl)benzaldehyde (6) (255 mg, 0.7 mmol, 1.0 eq) in DCM (5 mL) was added DAST (0.92 mL, 7.0 mmol, 10.0 eq) at −78° C. allowed to sir for 0.50 h at −78° C., another 3 h at RT. On completion, reaction mixture was quenched with saturated NaHCO₃ solution and extracted with DCM. Organic part was washed with brine, dried over Na₂SO₄, filtered and concentrated under reduced pressure to afford a yellowish crude. Obtained crude was purified by CombiFlash chromatography to afford 5-(3-(difluoromethyl)phenyl)-3-fluoro-5,8,8-trimethyl-5,8,9,10-tetrahydrobenzo[b][1,8]naphthyridin-6(7H)-one (7) (145 mg, 0.375 mmol, 100% purity, 54% yield) as light yellow solid.

LCMS: Rt=2.24 min, (ES⁺) m/z (M+H)⁺=387.2.

### Step 7: (S)-5-(3-(difluoromethyl)phenyl)-3-fluoro-5,8,8-trimethyl-5,8,9,10-tetrahydrobenzo[b][1,8]naphthyridin-6(7H)-one (7A) & (R)-5-(3-(difluoromethyl)phenyl)-3-fluoro-5,8,8-trimethyl-5,8,9,10-tetrahydrobenzo[b][1,8]naphthyridin-6(7H)-one (7B)

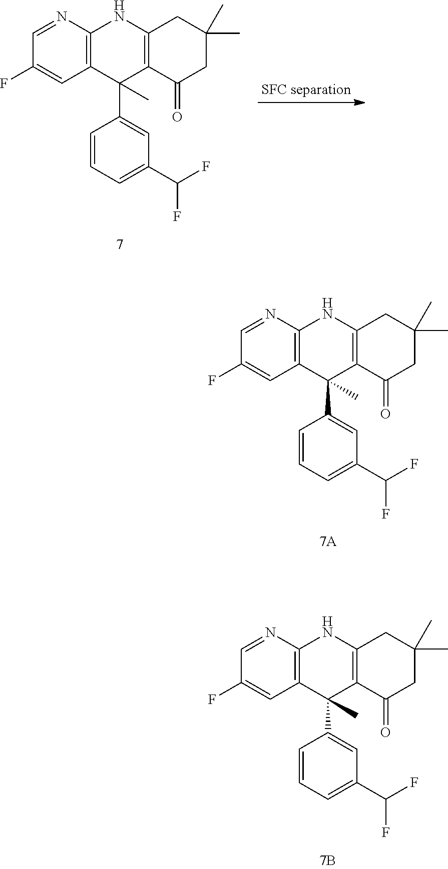 [SMILES: CC1(C)CC(=O)C2=C(C1)Nc1ncc(F)cc1C2(C)c1cccc(C(F)F)c1 ; CC1(C)CC(=O)C2=C(C1)Nc1ncc(F)cc1[C@]2(C)c1cccc(C(F)F)c1 ; CC1(C)CC(=O)C2=C(C1)Nc1ncc(F)cc1[C@@]2(C)c1cccc(C(F)F)c1]

Chiral separation of 5-(3-(difluoromethyl)phenyl)-3-fluoro-5,8,8-trimethyl-5,8,9,10-tetrahydrobenzo[b][1,8]naphthyridin-6(7H)-one (7) (145 mg, 0.375 mmol, 1.0 eq) was done by prep SFC purification on PIC SOLUTIONS-175 instrument equipped with Knauer 40D Detector by using Reflect (R,R)Whelk_O1 (21.1 mm×250 mm), 5μ Column operating at 35° C. temperature, maintaining flow rate of 60 ml/min, using 60% CO₂ in super critical state & 40%[100% MeOH] as Mobile phase. Run this isocratic mixture up to 10.0 minutes and also maintained the isobaric condition of 100 bar at 220 nm wavelength.

SFC fractions were collected and evaporated under reduced pressure to afford:

Peak 1: (S)-5-(3-(difluoromethyl)phenyl)-3-fluoro-5,8,8-trimethyl-5,8,9,10-tetrahydrobenzo[b][1,8]naphthyridin-6(7H)-one (7A), (47 mg, 0.117 mmol, 97.35% purity, 31% yield) as light orange solid.

UPLC: Rt=2.50 min, (ES⁺) m/z (M+H)⁺=387.2.

1H NMR (400 MHz, DMSO-d6) δ 9.96 (s, 1H), 8.00 (d, J=2.6 Hz, 1H), 7.53-7.52 (m, 2H), 7.39 (t, J=8.0 Hz, 1H), 7.29 (d, J=7.6 Hz, 1H), 7.02 (dd, J=9.4, 2.5 Hz, 1H), 6.98 (t, J=56 Hz, 1H), 2.49 (s, 2H, overlapped with DMSO signal), 2.07-1.92 (m, 2H), 1.95 (s, 3H), 1.01 (s, 3H), 0.99 (s, 3H).

19F NMR (376 MHz, DMSO-d6) d −109.05 (q, J=27.6 Hz), −134.99 (d, J=9.1 Hz).

[α]²⁵=+45.298, c=0.2517, MeOH.

Chiral purity (% ee): 100%

Peak 2: (R)-5-(3-(difluoromethyl)phenyl)-3-fluoro-5,8,8-trimethyl-5,8,9,10-tetrahydrobenzo[b][1,8]naphthyridin-6(7H)-one (7B) (37 mg, 0.0955 mmol, 98.58% purity, 25% yield) as light orange solid.

UPLC: Rt=2.50 min, (ES⁺) m/z (M+H)⁺=387.2.

1H NMR (400 MHz, DMSO-d6) δ 9.96 (s, 1H), 8.00 (d, J=2.6 Hz, 1H), 7.53-7.52 (m, 2H), 7.39 (t, J=8.0 Hz, 1H), 7.29 (d, J=7.6 Hz, 1H), 7.02 (dd, J=9.4, 2.5 Hz, 1H), 6.98 (t, J=56 Hz, 1H), 2.49 (s, 2H, overlapped with DMSO signal), 2.07-1.92 (m, 2H), 1.95 (s, 3H), 1.01 (s, 3H), 0.99 (s, 3H).

19F NMR (376 MHz, DMSO-d6) d −109.05 (q, J=27.7 Hz), −134.99 (d, J=9.2 Hz).

[α]²⁵=−51.023, c=0.2607, MeOH.

Chiral purity (% ee): 100%

### Example 3.14. (S)-5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11A) & (R)-5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11B)

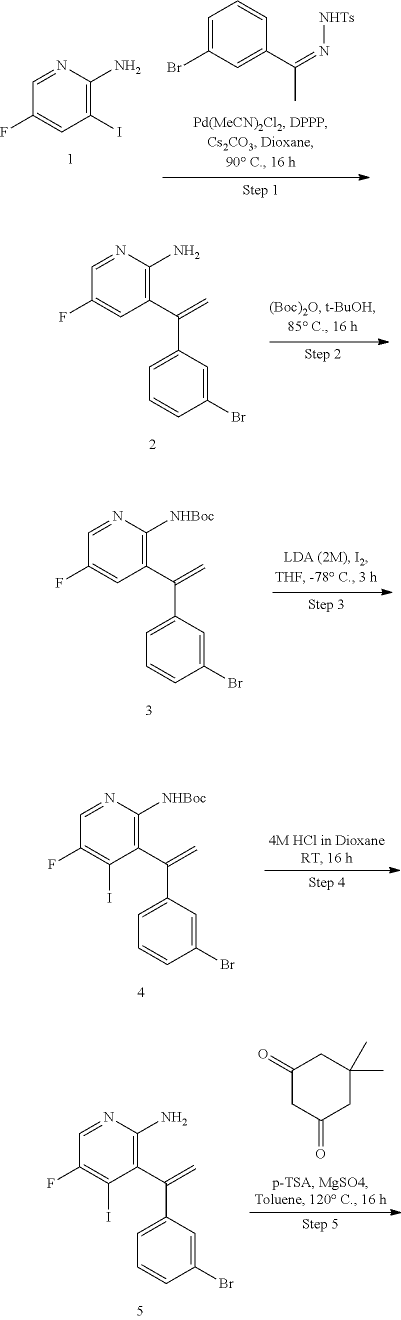

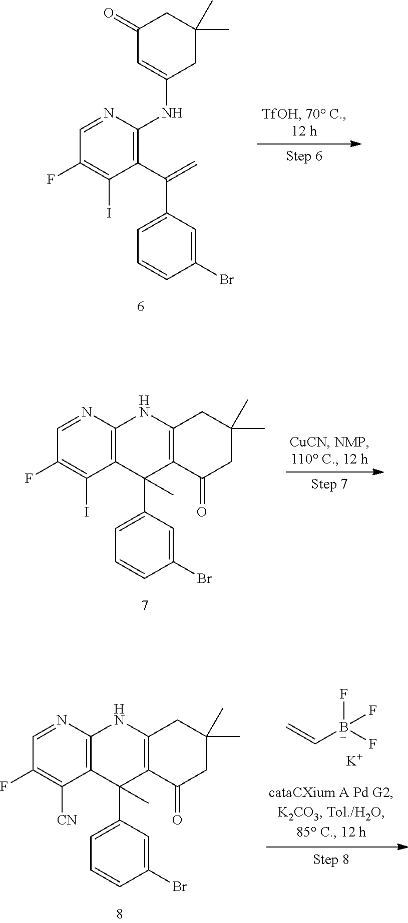

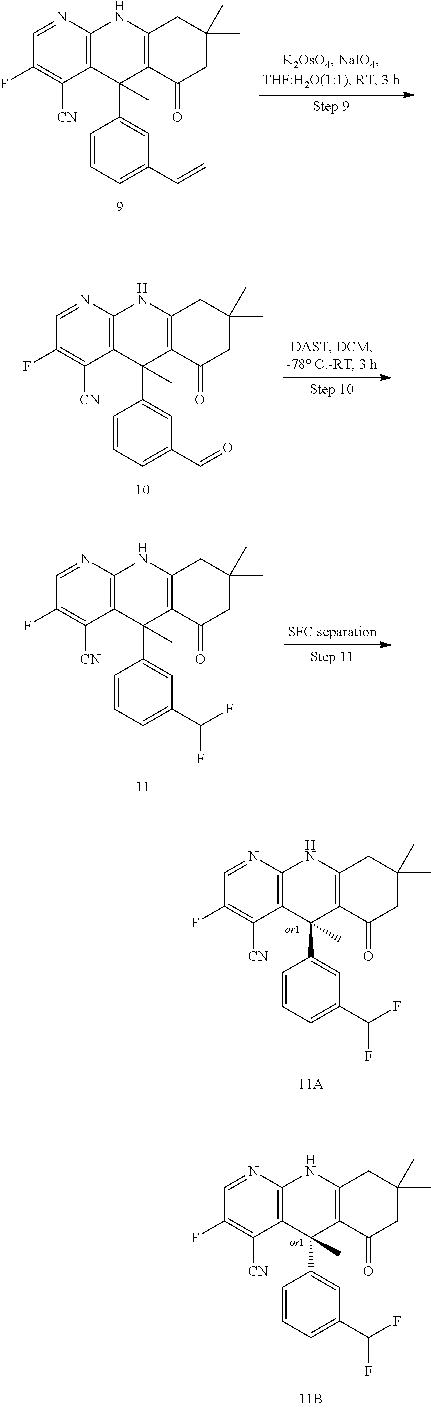 [SMILES: C=Cc1cccc(C2(C)C3=C(CC(C)(C)CC3=O)Nc3ncc(F)c(C#N)c32)c1 ; CC1(C)CC(=O)C2=C(C1)Nc1ncc(F)c(C#N)c1C2(C)c1cccc(C=O)c1 ; CC1(C)CC(=O)C2=C(C1)Nc1ncc(F)c(C#N)c1C2(C)c1cccc(C(F)F)c1 ; CC1(C)CC(=O)C2=C(C1)Nc1ncc(F)c(C#N)c1[C@]2(C)c1cccc(C(F)F)c1 ; CC1(C)CC(=O)C2=C(C1)Nc1ncc(F)c(C#N)c1[C@@]2(C)c1cccc(C(F)F)c1]

### Step 1: 3-[1-(3-bromophenyl)vinyl]-5-fluoro-pyridin-2-amine (2)

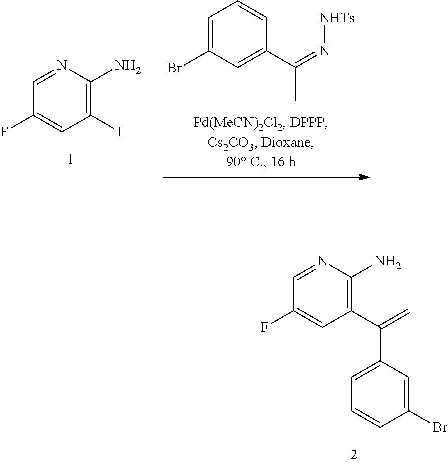

The solution of (Z)—N′-(1-(3-bromophenyl)ethylidene)-4-methylbenzenesulfonohydrazide (23.15 g, 63.0 mmol, 1.5 eq), DPPP (4.33 g, 10.5 mmol, 0.25 eq) and cesium carbonate (41.1 g, 126 mmol, 3 eq) in dioxane (600 mL) was bubbled with argon for 15 min, then Bis(acetonitrile)dichloro palladium (II) (1.64 g, 6.30 mmol, 0.15 eq) and 5-fluoro-3-iodo-pyridin-2-amine (1) (10 g, 42 mmol, 1.0 eq) were sequentially added. The reaction mixture was allowed to stir at 90° C. for 16 h. Reaction mixture was filtered through celite pad and washed with ethyl acetate. Obtained filtrate was then washed with water and brine, dried over sodium sulphate, filtered and concentrated under reduced pressure to afford a brownish crude. Obtained crude was purified by combiflash column chromatography using 5-9% EtOAc-hexane gradient to afford 3-[1-(3-bromophenyl)vinyl]-5-fluoro-pyridin-2-amine (2) (2.0 g, 6.82 mmol, 16% yield).

LCMS: Rt=1.99 min, (ES⁺) m/z (M+H)⁺=295.02.

### Step 2: tert-butyl (3-(1-(3-bromophenyl)vinyl)-5-fluoropyridin-2-yl)carbamate (3)

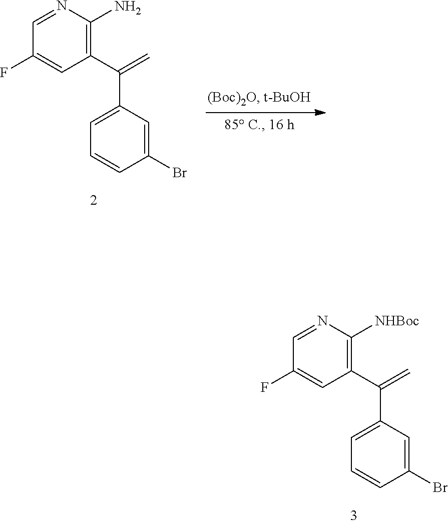 [SMILES: C=C(c1cccc(Br)c1)c1cc(F)cnc1N ; C=C(c1cccc(Br)c1)c1cc(F)cnc1NC(=O)OC(C)(C)C]

To the stirred solution of 3-[1-(3-bromophenyl)vinyl]-5-fluoro-pyridin-2-amine (2) (2.0 g, 6.82 mmol, 1.0 eq) in t-BuOH (15 mL) was added Boc₂O (3.92 mL, 17.1 mmol, 2.5 eq) dropwise, then reaction mixture was allowed to stir for 16 h at 85° C. Reaction mixture was diluted with ethyl acetate, which washed with water, brine, dried over anhydrous Na₂SO₄, filtered off and concentrated under reduced pressure to get a colorless gummy crude. Obtained crude was purified by CombiFlash column chromatography using 5-15% EtOAc in hexane gradient to afford tert-butyl (3-(1-(3-bromophenyl)vinyl)-5-fluoropyridin-2-yl)carbamate (3) (1.2 g, 2.56 mmol, 84% purity, 38% yield)

LCMS: Rt=2.12 min, (ES⁺) m/z (M+H)⁺=393.4.

### Step 3: tert-butyl (3-(1-(3-bromophenyl)vinyl)-5-fluoro-4-iodopyridin-2-yl)carbamate (4)

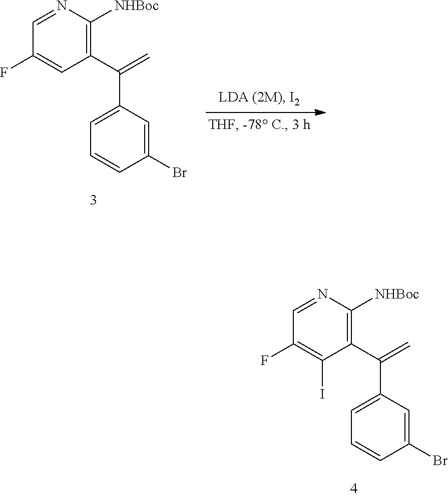 [SMILES: C=C(c1cccc(Br)c1)c1cc(F)cnc1NC(=O)OC(C)(C)C ; C=C(c1cccc(Br)c1)c1c(NC(=O)OC(C)(C)C)ncc(F)c1I]

To a stirred solution of tert-butyl (3-(1-(3-bromophenyl)vinyl)-5-fluoropyridin-2-yl)carbamate (3) (1.2 g, 3.05 mmol, 1.0 eq) in THF (15 mL) was dropwise added LDA (2 M in, 5.34 mL, 10.7 mmol, 3.5 eq) at −78° C. under inert atmosphere and stirred for 1 h under same condition. After that Iodine (1.16 g, 4.58 mmol, 1.5 eq) dissolved in THF (7 mL) was added dropwise to RM at −78° C., stirred for additional 1 h at −78° C., at 25° C. for another 1 h. Then reaction mixture was quenched with aqueous NH₄Cl solution and extracted with ethyl acetate Obtained ethyl acetate part was sequentially washed with aq. Na₂S₂O₃ solution, water and brine, dried over anhydrous Na₂SO₄, filtered off and concentrated under vacuo to afford a brownish crude. Obtained crude was purified by CombiFlash column chromatography to afford tert-butyl (3-(1-(3-bromophenyl)vinyl)-5-fluoro-4-iodopyridin-2-yl)carbamate (4) (620 mg, 1.05 mmol, 88% purity, 34% yield)

LCMS: Rt=3.87 min, (ES⁺) m/z (M+H)⁺=518.9.

### Step 4: 3-[1-(3-bromophenyl)vinyl]-5-fluoro-4-iodo-pyridin-2-amine. (5)

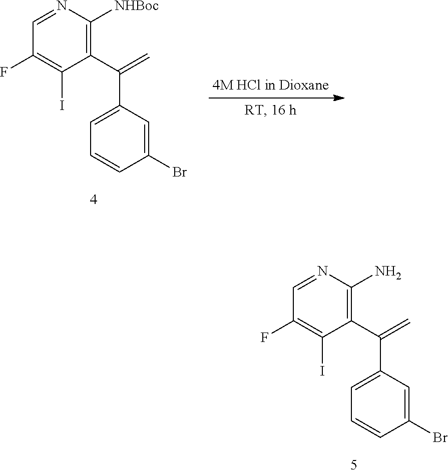 [SMILES: C=C(c1cccc(Br)c1)c1c(NC(=O)OC(C)(C)C)ncc(F)c1I ; C=C(c1cccc(Br)c1)c1c(N)ncc(F)c1I]

To a stirred solution of tert-butyl (3-(1-(3-bromophenyl)vinyl)-5-fluoro-4-iodopyridin-2-yl)carbamate (4) (620 mg, 1.19 mmol, 1.0 eq) in Dioxane (3 mL) was dropwise added 4 M HCl in 1,4 Dioxane (10 mL) at 0° C., then the resultant reaction mixture was allowed to stir at RT for 16 h. The reaction mixture was quenched with aqueous NaHCO₃ solution and extracted with ethyl acetate. Ethyl acetate part was washed with brine, dried over sodium sulphate, filtered off and concentrated under reduced pressure to afford crude 3-[1-(3-bromophenyl)vinyl]-5-fluoro-4-iodo-pyridin-2-amine (5) (450 mg, 1.07 mmol, 100% purity, 90% yield) as brownish solid. It was used as such for the next step without purification.

LCMS: Rt=3.72 min, (ES⁺) m/z (M+H)⁺=419.1.

### Step 5: 3-((3-(1-(3-bromophenyl)vinyl)-5-fluoro-4-iodopyridin-2-yl)amino)-5,5-dimethylcyclohex-2-en-1-one 6)

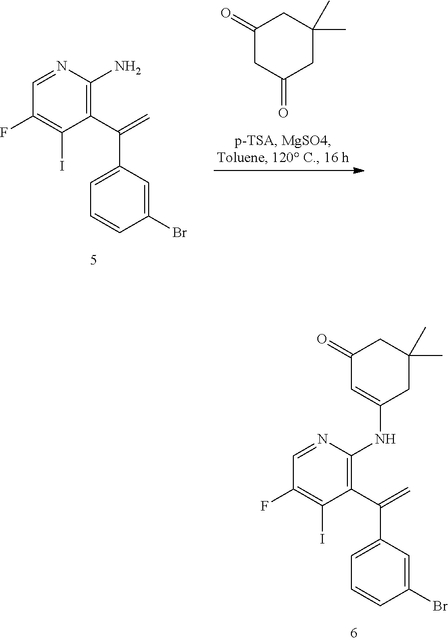

To the stirred solution of 3-[1-(3-bromophenyl)vinyl]-5-fluoro-4-iodo-pyridin-2-amine (5) (450 mg, 1.07 mmol, 1.0 eq) in toluene (12 mL) were added 5,5-dimethylcyclohexane-1,3-dione (226 mg, 1.61 mmol, 1.5 eq), MgSO₄ (1.3 g, 10.7 mmol, 10.0 eq) and pTSA (21 mg, 0.107 mmol, 0.1 eq) sequentially under inert atmosphere at RT. Resulting mixture was allowed to stir at 120° C. for 16 h. Reaction mixture was filtered through celite bed, washed with ethyl acetate, filtrate was collected and concentrated under reduced pressure to afford a brownish crude. Obtained crude was purified by CombiFlash column chromatography using 20-25% EA in hexane gradient to furnish 3-((3-(1-(3-bromophenyl)vinyl)-5-fluoro-4-iodopyridin-2-yl)amino)-5,5-dimethylcyclohex-2-en-1-one (6) (430 mg, 0.771 mmol, 97% purity, 72% yield) as off white solid.

LCMS: Rt=2.35 min, (ES⁺) m/z (M+H)⁺=543.2.

### Step 6: 5-(3-bromophenyl)-3-fluoro-4-iodo-5,8,8-trimethyl-5,8,9,10-tetrahydrobenzo[b][1,8]naphthyridin-6(7H)-one (7)

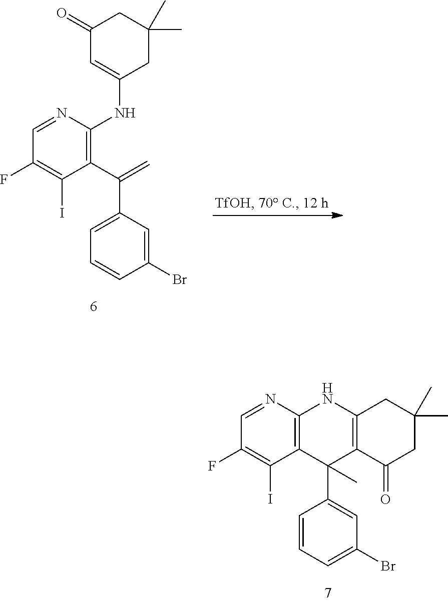 [SMILES: C=C(c1cccc(Br)c1)c1c(NC2=CC(=O)CC(C)(C)C2)ncc(F)c1I ; CC1(C)CC(=O)C2=C(C1)Nc1ncc(F)c(I)c1C2(C)c1cccc(Br)c1]

TfOH (10 mL) was added dropwise to a RB flask containing 3-((3-(1-(3-bromophenyl)vinyl)-5-fluoro-4-iodopyridin-2-yl)amino)-5,5-dimethylcyclohex-2-en-1-one (6) (430 mg, 0.795 mmol, 1.0 eq) at ice cold condition under inert atmosphere. Resulting mixture was allowed to stir at 70° C. for 12 h. Reaction mixture was quenched with aq. NaHCO₃ solution and extracted by ethyl acetate (twice). Combined organic layer was washed with brine, dried over anhydrous Na₂SO₄, filtered off and concentrated under vacuo to afford a brownish crude, which purified by CombifFlash chromatography using 10-30% ethyl acetate in hexane gradient to furnish 5-(3-bromophenyl)-3-fluoro-4-iodo-5,8,8-trimethyl-5,8,9,10-tetrahydrobenzo[b][1,8]naphthyridine-6(7H)-one (7) (350 mg, 0.634 mmol, 98% purity, 80% yield) as light-yellow solid.

LCMS: Rt=3.74 min, (ES⁺) m/z (M+H)⁺=540.9.

### Step 7: 5-(3-bromophenyl)-3-fluoro-5,8,8-trimethyl-6-oxo-5,6,7,8,9,10-hexahydrobenzo [b][1,8]naphthyridine-4-carbonitrile (8)

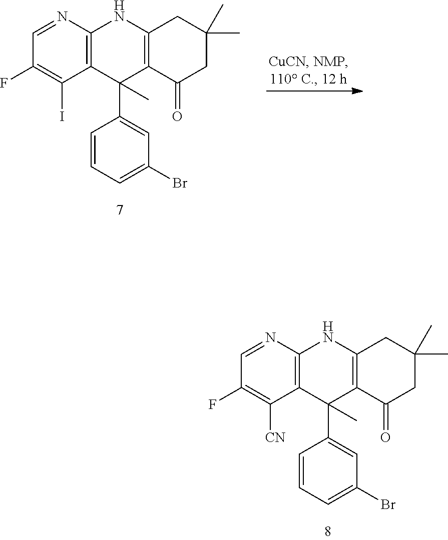 [SMILES: CC1(C)CC(=O)C2=C(C1)Nc1ncc(F)c(I)c1C2(C)c1cccc(Br)c1 ; CC1(C)CC(=O)C2=C(C1)Nc1ncc(F)c(C#N)c1C2(C)c1cccc(Br)c1]

To a stirred solution of 5-(3-bromophenyl)-3-fluoro-4-iodo-5,8,8-trimethyl-5,8,9,10-tetrahydrobenzo[b][1,8]naphthyridin-6(7H)-one (7) (350 mg, 0.647 mmol, 1.0 eq) in NMP (2.5 mL) was added CuCN (116 mg, 1.29 mmol, 2 eq) and stirred at 110° C. for 12 h in a sealed tube. On completion, reaction mixture was poured in ice cooled water and extracted with ethyl acetate (twice). Combined organic layer was washed with brine, dried over sodium sulfate, filtered and concentrated under reduced pressure to obtain a yellowish crude. Purification of resultant crude by CombiFlash column chromatography using 30-40% ethyl acetate in hexane gradient afforded 5-(3-bromophenyl)-3-fluoro-5,8,8-trimethyl-6-oxo-5,6,7,8,9,10-hexahydrobenzo [b][1,8]naphthyridine-4-carbonitrile (8) (160 mg, 0.363 mmol, 100% purity, 56% yield) as yellowish solid.

LCMS: Rt=3.53 min, (ES⁺) m/z (M+H)⁺=440.2.

### Step 8: 3-fluoro-5,8,8-trimethyl-6-oxo-5-(3-vinylphenyl)-5,6,7,8,9,10-hexahydrobenzo[b][1,8]naphthyridine-4-carbonitrile (9)

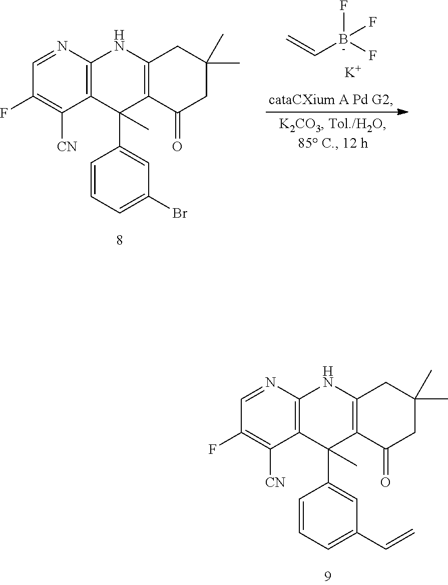

The solution of 5-(3-bromophenyl)-3-fluoro-5,8,8-trimethyl-6-oxo-5,6,7,8,9,10-hexahydrobenzo [b][1,8]naphthyridine-4-carbonitrile (8) (160 mg, 0.363 mmol, 1.0 eq), potassium trifluoro(vinyl)boranuide (73 mg, 0.545 mmol, 1.50 eq) and K₂CO₃ (100 mg, 0.727 mmol, 2.00 eq) in 4:1 toluene-water mixture (15 mL) was purged with argon gas for 10 min, then cataCXium A Pd G2 (24 mg, 0.0363 mmol, 0.1 eq) was added. The resulting mixture was allowed to stir at 85° C. for 12 h. Reaction mixture was diluted with EtOAc and washed sequentially with water and brine. Organic part was collected, dried over Na₂SO₄, filtered and concentrated under reduced pressure to afford a brownish crude, which was purified by CombiFlash column chromatography using 25-35% EtOAc in hexane gradient to afford 3-fluoro-5,8,8-trimethyl-6-oxo-5-(3-vinylphenyl)-5,6,7,8,9,10-hexahydrobenzo[b][1,8]naphthyridine-4-carbonitrile (9) (110 mg, 0.284 mmol, 78% yield).

LCMS: Rt=1.47 min, (ES⁺) m/z (M+H)⁺=388.3.

### Step 9: 3-fluoro-5-(3-formylphenyl)-5,8,8-trimethyl-6-oxo-5,6,7,8,9,10-hexahydrobenzo[b][1,8]naphthyridine-4-carbonitrile (10)

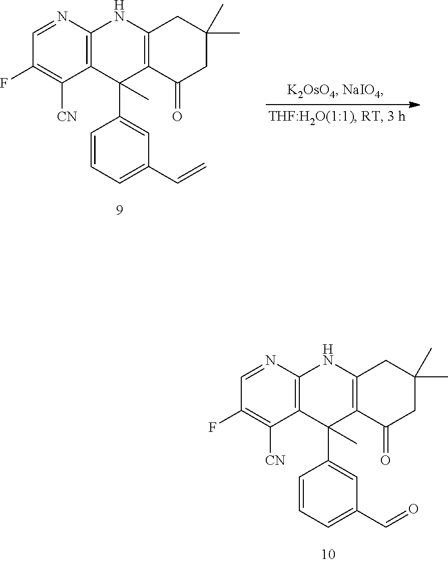 [SMILES: C=Cc1cccc(C2(C)C3=C(CC(C)(C)CC3=O)Nc3ncc(F)c(C#N)c32)c1 ; CC1(C)CC(=O)C2=C(C1)Nc1ncc(F)c(C#N)c1C2(C)c1cccc(C=O)c1]

To a stirred solution of 3-fluoro-5,8,8-trimethyl-6-oxo-5-(3-vinylphenyl)-5,6,7,8,9,10-hexahydrobenzo[b][1,8]naphthyridine-4-carbonitrile (9) (110 mg, 0.284 mmol, 1.0 eq) in 1:1 THF-H₂O mixture (6 mL) were added potassium osmate (4.7 mg, 0.0142 mmol, 0.05 eq) and sodium periodate (304 mg, 1.42 mmol, 5.0 eq) and the reaction mixture was allowed to stir for 3 h at RT. Reaction mixture was diluted with EtOAc and washed sequentially with water and brine, dried over Na₂SO₄, filtered off and concentrated under reduced pressure to afford a yellowish crude. Obtained crude was triturated with pentane to afford 3-fluoro-5-(3-formylphenyl)-5,8,8-trimethyl-6-oxo-5,6,7,8,9,10-hexahydrobenzo[b][1,8]naphthyridine-4-carbonitrile (10) (90 mg, 0.208 mmol, 90% purity, 73% yield) as light yellow solid. Obtained compound was used for next step without further purification.

LCMS: Rt=1.75 min, (ES⁺) m/z (M+H)⁺=390.2.

### Step 10: 5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11)

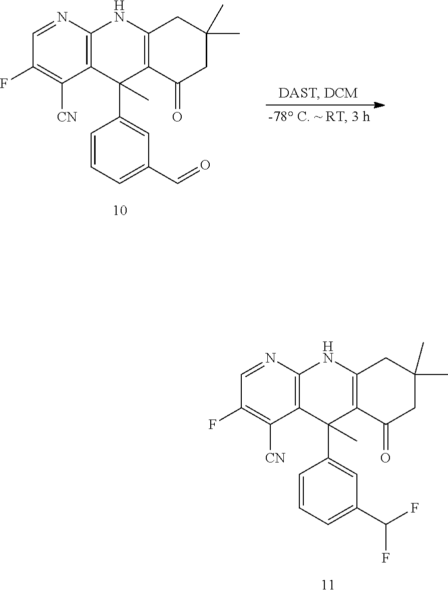 [SMILES: CC1(C)CC(=O)C2=C(C1)Nc1ncc(F)c(C#N)c1C2(C)c1cccc(C=O)c1 ; CC1(C)CC(=O)C2=C(C1)Nc1ncc(F)c(C#N)c1C2(C)c1cccc(C(F)F)c1]

To a stirred solution of 3-fluoro-5-(3-formylphenyl)-5,8,8-trimethyl-6-oxo-5,6,7,8,9,10-hexahydrobenzo[b][1,8]naphthyridine-4-carbonitrile (10) (90 mg, 0.231 mmol, 1.0 eq) in DCM (3 mL) was dropwise added DAST (0.31 mL, 2.31 mmol, 10 eq) at −78° C. under inert atmosphere. Resulting mixture allowed to stir for 0.5 h under same temperature, then at RT for additional 3 h. On completion, reaction mixture was quenched with saturated NaHCO₃ solution and extract with DCM. Obtained organic layer was dried over Na₂SO₄, filtered and concentrated under reduced pressure to afford a yellow crude, which was purified by CombiFlash chromatography using 20-40% EtOAc in hexane gradient to afford 5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11) (60 mg, 0.140 mmol, 96% purity, 61% yield) as light yellow solid.

LCMS: Rt=2.65 min, (ES⁺) m/z (M+H)⁺=412.2.

### Step 11: (S)-5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11A) & (R)-5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11B)

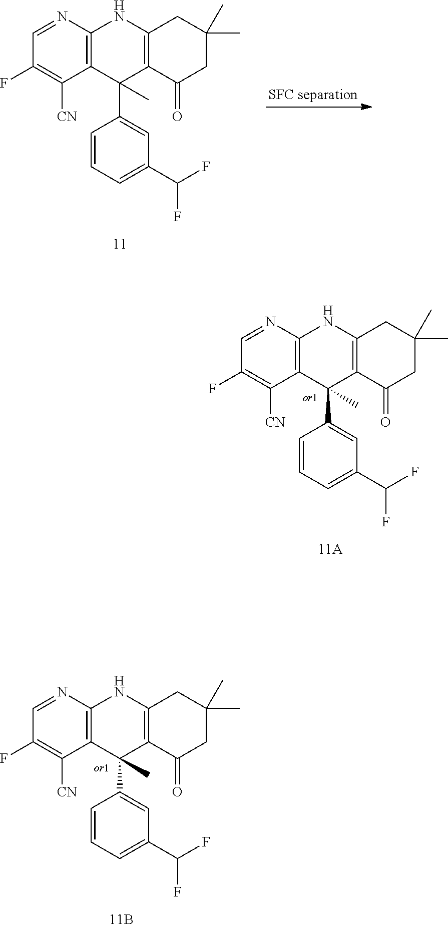 [SMILES: CC1(C)CC(=O)C2=C(C1)Nc1ncc(F)c(C#N)c1C2(C)c1cccc(C(F)F)c1 ; CC1(C)CC(=O)C2=C(C1)Nc1ncc(F)c(C#N)c1[C@]2(C)c1cccc(C(F)F)c1 ; CC1(C)CC(=O)C2=C(C1)Nc1ncc(F)c(C#N)c1[C@@]2(C)c1cccc(C(F)F)c1]

Chiral separation of 5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11) (60 mg, 0.146 mmol, 1.0 eq) was done on SFC 80 instrument equipped with Waters 2489 UV/Visible Detector by using Reflect(R,R)Whelk_O1 (21.1 mm×250 mm), 5μ Column operating at 35° C. temperature, maintaining flow rate of 70 ml/min, using 70% CO₂ in super critical state & 30%[10(0% Isopropyl Alcohol] as Mobile phase, Run this isocratic mixture up to 7.0 minutes and also maintained the isobaric condition of 100 bar at 220 nm wavelength.

SFC fractions were collected and evaporated under vacuo to afford:

Peak 1: (S)-5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11A) (23 mg, 0.0514 mmol, 92% purity, 35% yield) as yellow solid

UPLC: Rt=2.45 min, (ES⁺) m/z (M+H)⁺=412.2.

1H NMR (400 MHz, DMSO-d6) δ 10.31 (s, 1H), 8.43 (s, 1H), 7.57-7.53 (m, 2H), 7.40-7.32 (m, 2H), 6.98 (t, J=55.84 Hz, 1H), 2.46-2.45 (m, 2H), 2.15 (s, 3H), 2.03-1.86 (m, 2H), 0.99 (s, 3H), 0.91 (s, 3H).

[α]²⁵=+269.352, c=0.1786, MeOH.

Chiral purity (% ee): 99.68

Peak 2: (R)-5-(3-(difluoromethyl)phenyl)-3-fluoro-5,8,8-trimethyl-6-oxo-5,6,7,8,9,10-hexahydrobenzo[b][1,8]naphthyridine-4-carbonitrile (11B). (20 mg, 0.0481 mmol, 98.72% purity, 33% yield) as yellow solid.

UPLC: Rt=2.45 min, (ES⁺) m/z (M+H)⁺=412.2.

1H NMR (400 MHz, DMSO-d6) δ 10.31 (s, 1H), 8.43 (s, 1H), 7.57-7.53 (m, 2H), 7.40-7.32 (m, 2H), 6.98 (t, J=55.84 Hz, 1H), 2.46-2.45 (m, 2H), 2.15 (s, 3H), 2.03-1.86 (m, 2H), 0.99 (s, 3H), 0.91 (s, 3H).

[α]²⁵=−278.351, c=0.1829, MeOH.

Chiral purity (% ee): 99.84

### Example 3.15. (S)-3-fluoro-5,8,8-trimethyl-5-(3-(3-(2,2,2-trifluoroethyl)pyridin-4-yl)phenyl)-5,8,9,10-tetrahydrobenzo[b][1,8]naphthyridin-6(7H)-one (A) & (R)-3-fluoro-5,8,8-trimethyl-5-(3-(3-(2,22-trifluoroethyl)pyridin-4-yl)phenyl-5,8,9,10-tetrahydrobenzo[b][1,8]naphthyridin-6(7H)-one (B)

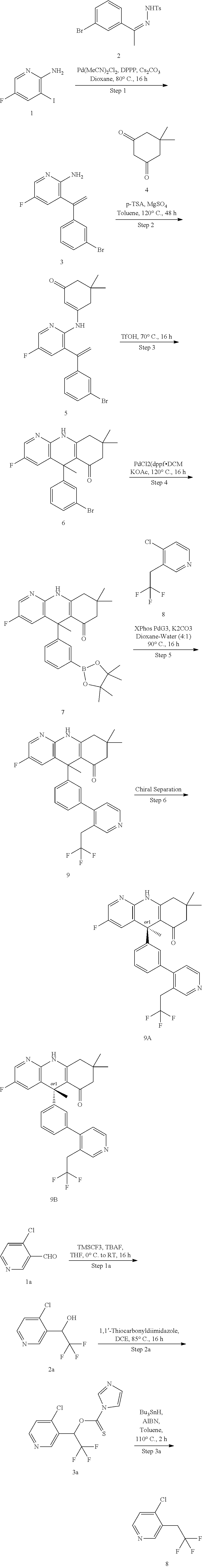

### Step 1: 3-(1-(3-bromophenyl) vinyl)-5-fluoropyridin-2-amine (3)

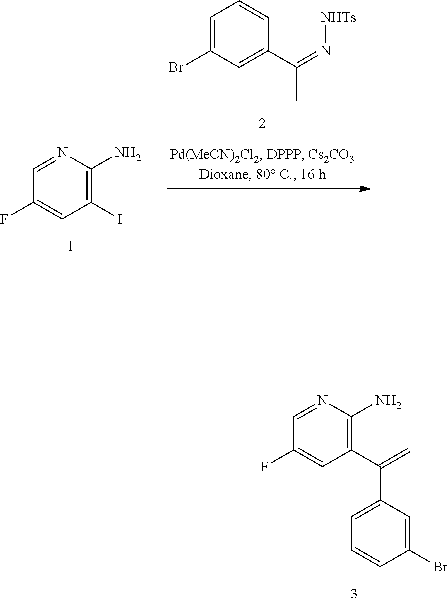

To a stirred solution of N-(1-(3-bromophenyl)ethylidene)-4-methylbenzenesulfonohydrazide (10.80 g, 29.4 mmol, 1.40 eq) and 5-fluoro-3-iodo-pyridin-2-amine (5.00 g, 21.0 mmol, 1.00 eq) in Dioxane (300 mL) was added Cs2CO3 (17.11 g, 52.5 mmol, 2.50 eq) under inert atmosphere. The resulting mixture was degassed with argon for 10 mins followed by addition of DPPP (2.60 g, 6.30 mmol, 0.300 eq) and Bis(acetonitrile)dichloro palladium (II) (0.82 g, 3.15 mmol, 0.150 eq). Reaction mixture was then allowed to stir at 80° C. for 16 h. The reaction mixture was then filtered through celite pad and washed with EtOAc (500 mL). The filtrate was diluted with water (100 mL) and extracted with EtOAc (2×300 mL). Combined organic layer was washed with water and brine. Organic layer was dried over Na2SO4, filtered and concentrated under reduced pressure to get crude product which was purified by combi-flash chromatography (Column: SiO2, 40 g) using 5%-7% EtOAc in Hexane to provide 3-(1-(3-bromophenyl) vinyl)-5-fluoropyridin-2-amine (1.00 g, 2.59 mmol, 76% purity, 12% yield) yellow solid.

LCMS: (ES⁺) m/z (M+H)⁺=295.2, Rt=3.54 min

### Step 2: 3-((3-(1-(3-bromophenyl) vinyl)-5-fluoropyridin-2-yl) amino)-5,5-dimethylcyclohex-2-en-1-one (S)

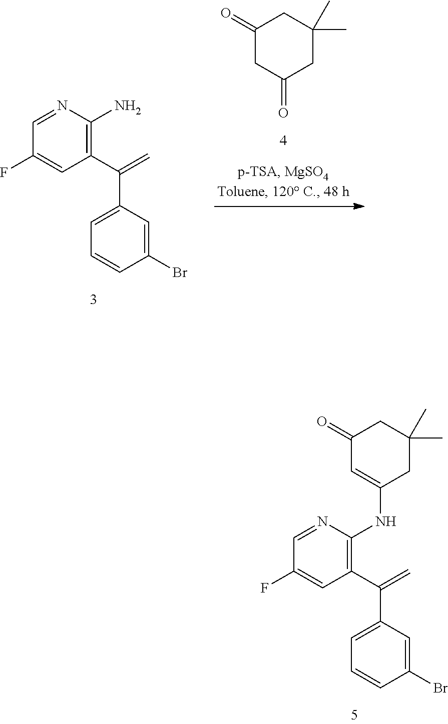

To the stirred solution of 3-[1-(3-bromophenyl)vinyl]-5-fluoro-pyridin-2-amine (1 g, 3.41 mmol, 1.00 eq) in Toluene (12 mL) were sequentially added 5,5-dimethylcyclohexane-1,3-dione (717 mg, 5.12 mmol, 1.50 eq), MgSO4 (4.1 mg, 34.1 mmol, 10.0 eq) and p-TSA (65 mg, 0.341 mmol, 0.100 eq) under inert atmosphere at RT. Resulting mixture was allowed to stir at 120° C. for 48 h. Reaction mixture was filtered over a celite pad washed with EtOAc (100 mL). The filtrate was evaporated under reduced pressure to provide crude product which was purified by silica gel Combi flash chromatography (Column: SiO2, 12 g) using 10-20% EtOAc in Hexane to provide 3-[[3-[1-(3-bromophenyl) vinyl]-5-fluoro-2-pyridyl]amino]-5,5-dimethyl-cyclohex-2-en-1-one (650 mg, 1.49 mmol, 95% purity, 44% yield) as yellow solid.

LCMS: (ES⁺) m/z (M+H)⁺=414.7 and 416.7 (bromo pattern), Rt=3.48 min

### Step 3: 5-(3-bromophenyl)-3-fluoro-5,8,8-trimethyl-5,8,9,10-tetrahydrobenzo[b][1,8]naphthyridin-6(7H)-one (6)

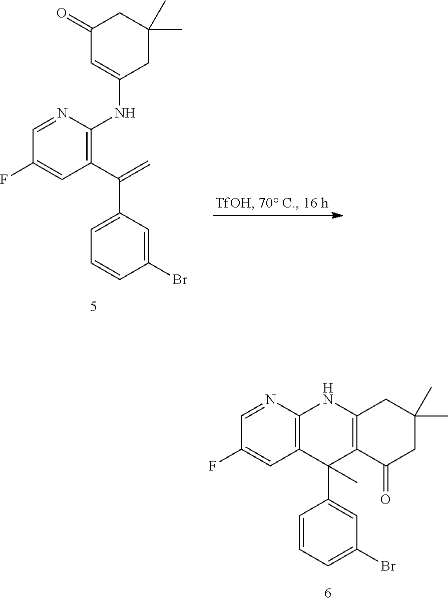 [SMILES: C=C(c1cccc(Br)c1)c1cc(F)cnc1NC1=CC(=O)CC(C)(C)C1 ; CC1(C)CC(=O)C2=C(C1)Nc1ncc(F)cc1C2(C)c1cccc(Br)c1]

TfOH (6 mL) was added drop wise to 3-[[3-[1-(3-bromophenylvinyl]-5-fluoro-2-pyridylamino]-5,5-dimethyl-cyclohex-2-en-1-one (650 mg, 1.57 mmol, 1.00 eq) at 0° C. under inert atmosphere. The resulting solution was allowed to stir for 16 h at 70° C. Reaction was monitored by TLC (nonpolar spot with respect to starting material). The reaction mixture was dropwise poured to ice cooled aqueous solution of NaHCO3 and extract with DCM (2×100 mL). Combined organic layer was washed with brine. The organic layer was dried over Na2SO4, filtered and concentrated under reduced pressure to get crude product which was purified by Combi flash chromatography (Column: SiO2, 12 g) using 40% EtOAc in Hexane to provide 5-(3-bromophenyl)-3-fluoro-5,8,8-trimethyl-9,10-dihydro-7H-benzo[b][1,8]naphthyridin-6-one (410 mg, 0.987 mmol, 100% purity, 63% yield) as light brownish solid.

LCMS: (ES⁺) m/z (M+H)⁺=414.9 and 416.9 (bromo pattern), Rt=3.53 min

### Step 4: 3-fluoro-5,8,8-trimethyl-5-(3-(4,4,5,5-tetramethyl-1,3,2-dioxaborolan-2-yl) phenyl)-5,8,9,10-tetrahydrobenzo[b][1,8]naphthyridin-6(7H)-one (7)

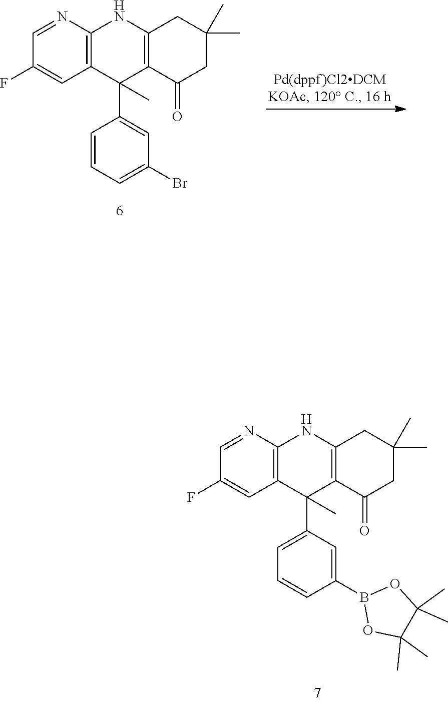 [SMILES: CC1(C)CC(=O)C2=C(C1)Nc1ncc(F)cc1C2(C)c1cccc(Br)c1 ; CC1(C)CC(=O)C2=C(C1)Nc1ncc(F)cc1C2(C)c1cccc(B2OC(C)(C)C(C)(C)O2)c1]

To a stirred solution of 5-(3-bromophenyl)-3-fluoro-5,8,8-trimethyl-9,10-dihydro-7H-benzo[b][1,8]naphthyridin-6-one (150 mg, 0.361 mmol, 1.00 eq) in dry Dioxane (5 mL) were added B2Pin2 (457 mg, 1.81 mmol, 5.00 eq) and dry KOAc (177 mg, 1.81 mmol, 5.00 eq). Reaction mixture was purged with argon for 15 mins. Then Pd(dppf)Cl₂·DCM (59 mg, 0.0722 mmol, 0.200 eq) was added to the reaction mixture and stirred for 16 h at 120° C. Reaction mixture was diluted with water (3 mL) and extracted with EtOAc (2×50 ml). Combined organic layer was dried over Na2SO4, filtered and concentrated under reduced pressure to get crude product which was purified by Combi flash column chromatography (column: SiO2, 4 g) using 30% EtOAc in Hexane to provide 3-fluoro-5,8,8-trimethyl-5-[3-(4,4,5,5-tetramethyl-1,3,2-dioxaborolan-2-yl)phenyl]-9,10-dihydro-7H-benzo[b][1,8]naphthyridin-6-one (75 mg, 0.162 mmol, 96% purity, 43% yield) as brownish solid.

LCMS: (ES+) m/z (M+H)⁺=463.2, Rt=2.08 min

### Step 5: 3-fluoro-5,8,8-trimethyl-5-(3-(3-(2,2,2-trifluoroethyl)pyridin-4-yl)phenyl)-5,8,9,10-tetrahydrobenzo[b][1,8]naphthyridin-6(7H)-one (9)

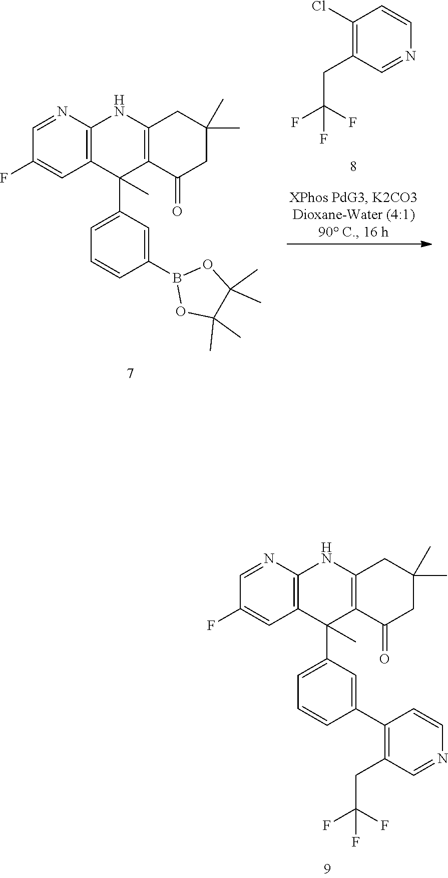 [SMILES: CC1(C)CC(=O)C2=C(C1)Nc1ncc(F)cc1C2(C)c1cccc(B2OC(C)(C)C(C)(C)O2)c1 ; FC(F)(F)Cc1cnccc1Cl ; CC1(C)CC(=O)C2=C(C1)Nc1ncc(F)cc1C2(C)c1cccc(-c2ccncc2CC(F)(F)F)c1]

To a stirred solution of 3-fluoro-5,8,8-trimethyl-5-[3-(4,4,5,5-tetramethyl-1,3,2-dioxaborolan-2-yl)phenyl]-9,10-dihydro-7H-benzo[b][1,8]naphthyridin-6-one (130 mg, 0.281 mmol, 1.00 eq) and 4-chloro-3-(2,2,2-trifluoroethyl)pyridine (110 mg, 0.562 mmol, 2.00 eq) in Dioxane:water (4:1) (4 mL) was added K₂CO₃ (117 mg, 0.843 mmol, 3.00 eq). Reaction mixture was purged with argon for 10 mins. Then XPhos Pd G3 (24 mg, 0.0281 mmol, 0.100 eq) was added and stirred at 90° C. for 16 h. Reaction mixture was diluted with water (2 mL) and extracted with EtOAc (2×25 ml). Combined organic layer was dried over Na2SO4, filtered and concentrated under reduced pressure to get crude product which was purified by combi flash column chromatography (Column: SiO2, 4 g) using 30-40% EtOAc in Hexane to provide 3-fluoro-5,8,8-trimethyl-5-[3-[3-(2,2,2-trifluoroethyl)-4-pyridyl]phenyl]-9,10-dihydro-7H-benzo[b][1,8]naphthyridin-6-one, (65 mg, 0.131 mmol, 99% purity, 47% yield) as off white solid.

LCMS: (ES+) m/z (M+H)⁺=496.2, Rt=2.19 min

### Step 6: (S)-3-fluoro-5,8,8-trimethyl-5-(3-(3-(2,2,2-trifluoroethyl) pyridin-4-yl) phenyl)-5,8,9,10-tetrahydrobenzo[b][1,8] naphthyridin-6(7H)-one (9A) & (R)-3-fluoro-5,8,8-trimethyl-5-(3-(3-(2,2,2-trifluoroethyl) pyridin-4-yl) phenyl)-5,8,9,10-tetrahydrobenzo[b][1,8]naphthyridin-6(7H)-one (9B)

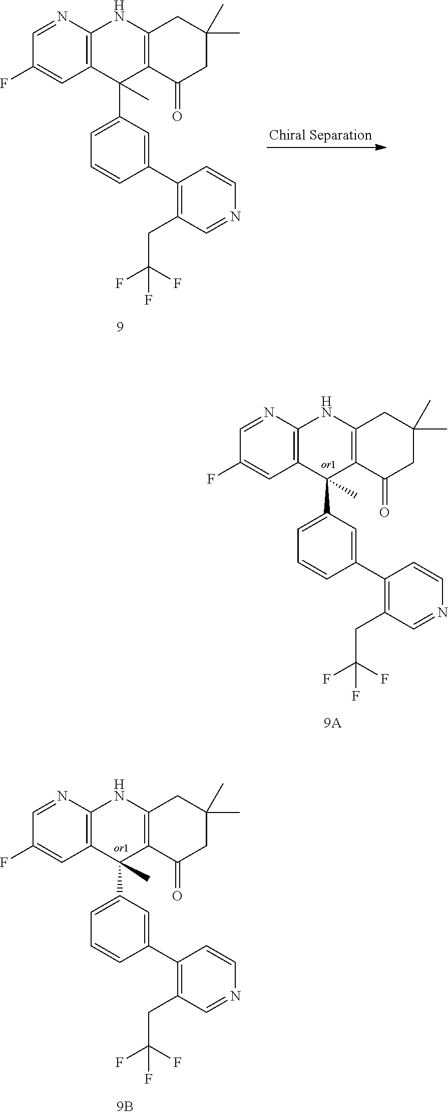 [SMILES: CC1(C)CC(=O)C2=C(C1)Nc1ncc(F)cc1C2(C)c1cccc(-c2ccncc2CC(F)(F)F)c1 ; CC1(C)CC(=O)C2=C(C1)Nc1ncc(F)cc1[C@]2(C)c1cccc(-c2ccncc2CC(F)(F)F)c1 ; CC1(C)CC(=O)C2=C(C1)Nc1ncc(F)cc1[C@@]2(C)c1cccc(-c2ccncc2CC(F)(F)F)c1]

The product was separated by SFC (Column: (R,R) WHELK-O 1 (21.1 mm×250 mm), 5μ; Mobile phase: 65% CO₂+35% of 100% IPA. Flow rate: 70 mL/min.

Peak 1: (S)-3-fluoro-5,8,8-trimethyl-5-(3-(3-(2,2,2-trifluoroethyl) pyridin-4-yl) phenyl)-5,8,9,10-tetrahydrobenzo[b][1,8]naphthyridin-6(7H)-one (25.48 mg, 99.66% purity, 27% yield) as off white solid.

LCMS: Rt=2.15 (ES+) m/z (M+H)=496.2

¹H NMR (400 MHz, DMSO-d6) δ 9.91 (s, 1H), 8.63 (s, 1H), 8.57 (d, J=5.2 Hz, 1H), 7.99 (d, J=2.8 Hz, 1H), 7.45-7.38 (m, 2H), 7.31-7.29 (m, 2H), 7.08 (d, J=7.2 Hz, 1H), 7.01 (dd, J=9.6 Hz, 9.6 Hz, 1H), 3.67-3.53 (m, 2H), 2.45 (s, 2H), 2.00 (d, J=9.6 Hz, 1H), 1.93 (s, 3H), 1.00 (s, 3H), 0.97 (s, 3H).

[α]²⁵=+103.735, c=0.1070, MeOH.

Chiral purity (ee %): 100

Peak 2: (R)-3-fluoro-5,8,8-trimethyl-5-(3-(3-(2,2,2-trifluoroethyl) pyridin-4-yl) phenyl)-5,8,9,10-tetrahydrobenzo[b][1,8] naphthyridin-6(7H)-one (32.86 mg, 99.87% purity, 35% yield) as off white solid.

LCMS: Rt=2.14 (ES+) m/z (M+H)=496.2

¹H NMR (400 MHz, DMSO-d6) δ 9.91 (s, 1H), 8.63 (s, 1H), 8.57 (d, J=5.2 Hz, 1H), 7.99 (d, J=2.8 Hz, 1H), 7.45-7.38 (m, 2H), 7.31-7.29 (m, 2H), 7.08 (d, J=7.2 Hz, 1H), 7.01 (dd, J=9.6 Hz, 9.6 Hz, 1H), 3.67-3.53 (m, 2H), 2.45 (s, 2H), 2.00 (d, J=9.6 Hz, 1H), 1.93 (s, 3H), 1.00 (s, 3H), 0.97 (s, 3H).

[α]²⁵=−166.708, c=0.1086, MeOH.

Chiral purity (ee %): 100

### Example 3.16.4-chloro-3-(2,2,2-trifluoroethyl) pyridine (8)

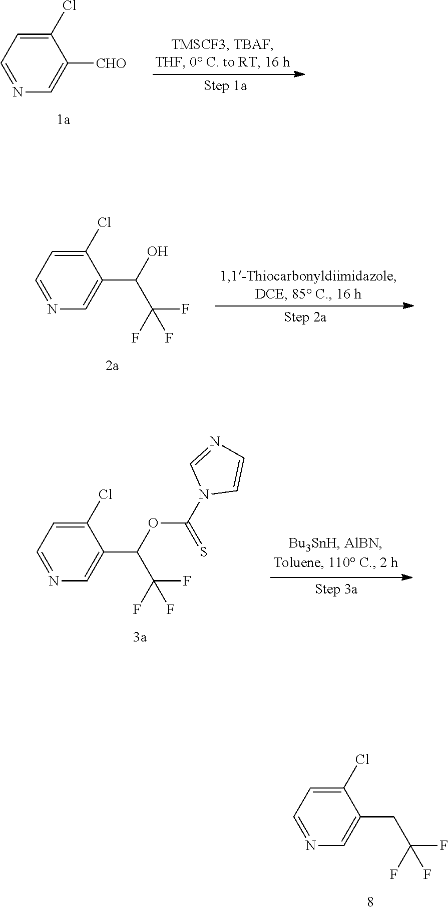 [SMILES: O=Cc1cnccc1Cl ; OC(c1cnccc1Cl)C(F)(F)F ; FC(F)(F)C(OC(=S)n1ccnc1)c1cnccc1Cl ; FC(F)(F)Cc1cnccc1Cl]

Step 1a: 1-(4-chloropyridin-3-yl)-2,2,2-trifluoroethan-1-ol (2a)

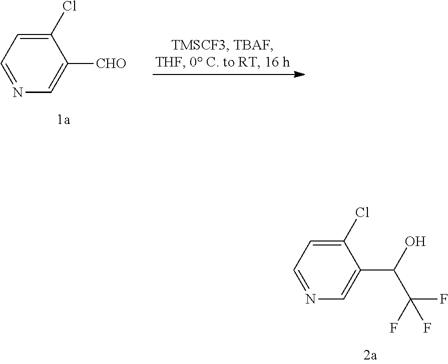 [SMILES: O=Cc1cnccc1Cl ; OC(c1cnccc1Cl)C(F)(F)F]

To a stirred solution of 4-chloropyridine-3-carbaldehyde (2.00 g, 14.1 mmol, 1.00 eq) in THF (45 mL) were added TMSCF3 (4.2 mL, 28.4 mmol, 2.01 eq) and TBAF (1 M in THF, 21.19 mL, 21.2 mmol, 1.50 eq) dropwise at 00 C. Reaction mixture was stirred at RT for 16 h. Reaction mixture was quenched with aqueous NaHCO₃ solution and extracted with EtOAc (3×100 mL). Combined organic layer was washed with brine. Organic layer was dried over Na2SO4, filtered and concentrated under reduced pressure to get crude product which was purified by combi flash column chromatography using in 25% EtOAc in hexane to provide 1-(4-chloro-3-pyridyl)-2,2,2-trifluoro-ethanol, (2.00 g, 9.42 mmol, 99.68% purity, 67% yield) as off white solid.

LCMS: (ES+) m/z (M+H)⁺=212.1, Rt=1.53 min

### Step 2a: 0-(1-(4-chloropyridin-3-yl)-2,2,2-trifluoroethyl) 1H-imidazole-1-carbothioate (3a)

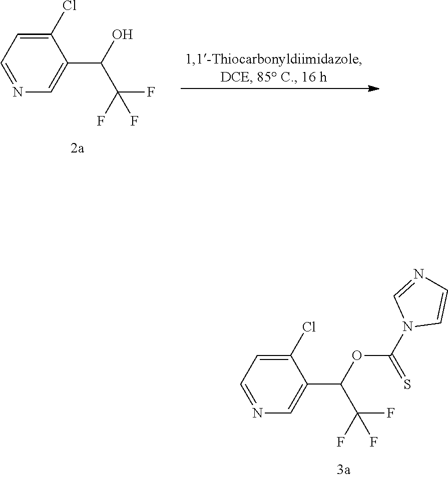 [SMILES: OC(c1cnccc1Cl)C(F)(F)F ; FC(F)(F)C(OC(=S)n1ccnc1)c1cnccc1Cl]

To stirred solution of 1-(4-chloro-3-pyridyl)-2,2,2-trifluoro-ethanol (2.00 g, 9.45 mmol, 1.00 eq) in DCE (40 mL) was added 1,1′-Thiocarbonyldiimidazole (2.52 g, 14.2 mmol, 1.50 eq). Reaction mixture was stirred at 85° C. for 16 h. Reaction mixture was concentrated under reduced pressure to provide O-(1-(4-chloropyridin-3-yl)-2,2,2-trifluoroethyl) 1H-imidazole-1-carbothioate (2.5 g, 82% yield) and crude compound was forwarded for next step without further purification.

LCMS: (ES+) m/z (M+H)⁺=322.1, Rt=2.63 min

### Step 3a: 4-chloro-3-(2,2,2-trifluoroethyl) pyridine (8)

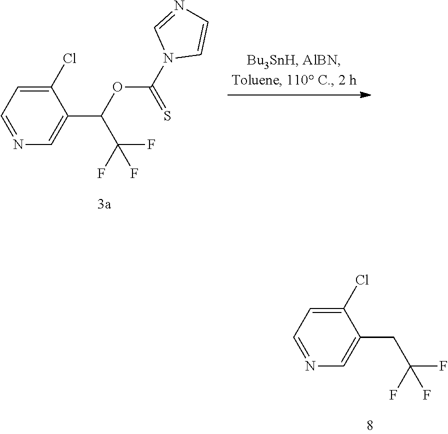 [SMILES: FC(F)(F)C(OC(=S)n1ccnc1)c1cnccc1Cl ; FC(F)(F)Cc1cnccc1Cl]

To a stirred solution of 0-[1-(4-chloro-3-pyridyl)-2,2,2-trifluoro-ethyl]imidazole-1-carbothioate (4.00 g, 12.4 mmol, 1.00 eq) in Toluene (100 mL) were added AIBN (408 mg, 2.49 mmol, 0.200 eq) and Bu3SnH (5.02 mL, 18.7 mmol, 1.50 eq) at 00 C. Reaction mixture was at 110° C. for 2 h. The reaction mixture was concentrated under reduced pressure to get crude product which was purified by combi flash column chromatography using 20% EtOAc in hexane to provide 4-chloro-3-(2,2,2-trifluoroethyl) pyridine, (2.10 g, 10.6 mmol, 99.03% purity, 86% yield) as colorless oil.

LCMS: (ES+) m/z (M+H)+=196.1, Rt=1.68 min

### Example 3.17. (S)-3-fluoro-5,8,8-trimethyl-6-oxo-5-(3-(3-(2,2,2-trifluoroethyl) pyridin-4-yl) phenyl)-5,6,7,8,9,10-hexahydrobenzo[b][1,8] naphthyridine-4-carbonitrile (8A) & (R)-3-fluoro-5,8,8-trimethyl-6-oxo-5-(3-(3-(2,2,2-trifluoroethyl) pyridin-4-yl) phenyl)-5,6,7,8,9,10-hexahydrobenzo[b][1,8] naphthyridine-4-carbonitrile (8B)

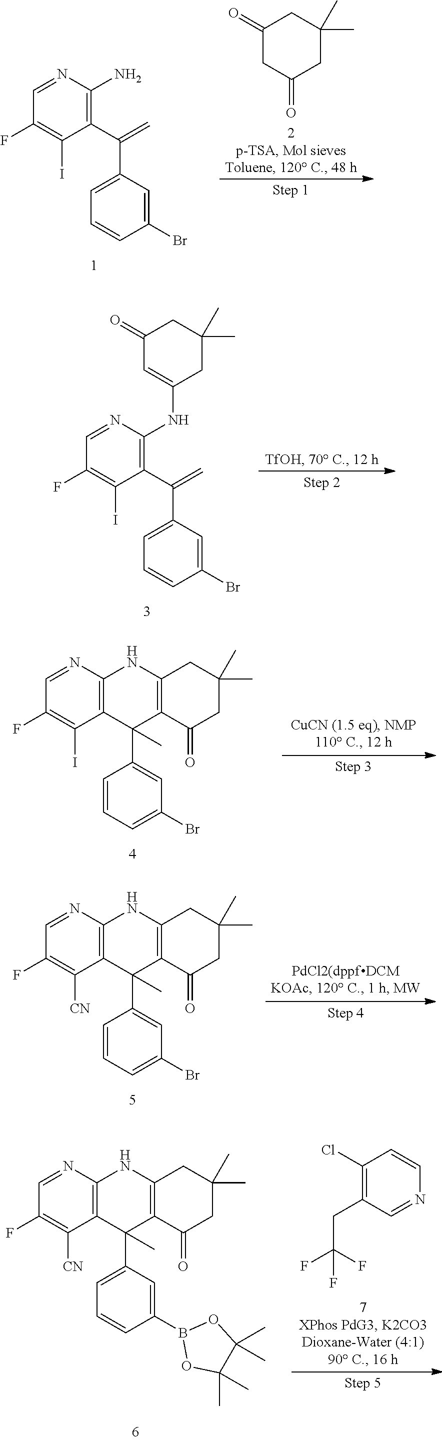

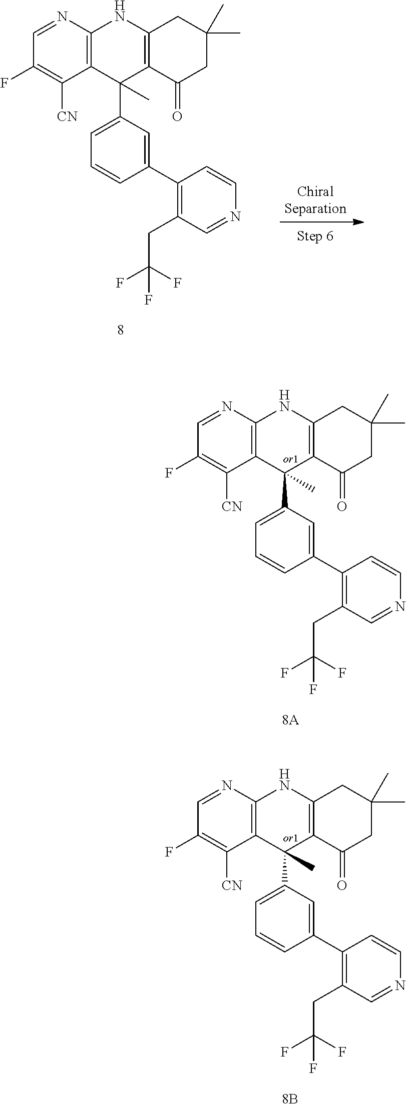 [SMILES: CC1(C)CC(=O)C2=C(C1)Nc1ncc(F)c(C#N)c1C2(C)c1cccc(-c2ccncc2CC(F)(F)F)c1 ; CC1(C)CC(=O)C2=C(C1)Nc1ncc(F)c(C#N)c1[C@]2(C)c1cccc(-c2ccncc2CC(F)(F)F)c1 ; CC1(C)CC(=O)C2=C(C1)Nc1ncc(F)c(C#N)c1[C@@]2(C)c1cccc(-c2ccncc2CC(F)(F)F)c1]

### Step 1: 3-((3-(1-(3-bromophenyl)vinyl)-5-fluoro-4-iodopyridin-2-yl)amino)-5,5-dimethylcyclohex-2-en-1-one (3)

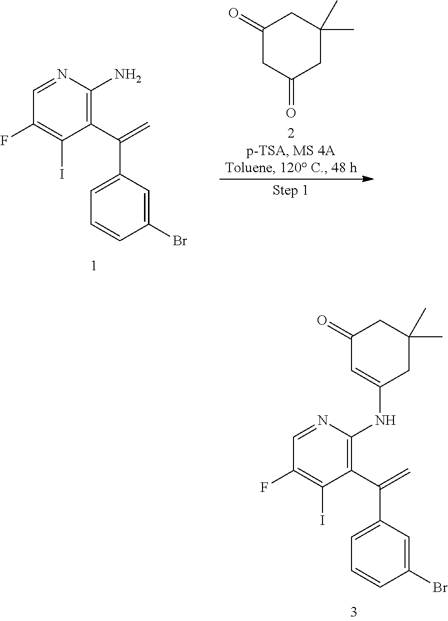

To a stirred solution of 3-[1-(3-bromophenyl)vinyl]-5-fluoro-4-iodo-pyridin-2-amine (1) (460 mg, 1.10 mmol, 1.00 eq) in Toluene (10 mL) were added 5,5-dimethylcyclohexane-1,3-dione (2) (231 mg, 1.65 mmol, 1.50 eq), MS 4A (400 mg) and p-TSA (21 mg, 0.110 mmol, 0.100 eq). Reaction mixture was stirred at 120° C. for 48 h. Reaction mixture was filtered through celite bed and celite bed was washed with EtOAc (50 mL). Filtrate was concentrated under reduced pressure to get crude which was purified by combi flash column chromatography (Column: SiO2, 12 g) using 20-25% EA in Hexane to provide 3-[[3-[1-(3-bromophenyl)vinyl]-5-fluoro-4-iodo-2-pyridyl]amino]-5,5-dimethyl-cyclohex-2-en-1-one (3) (425 mg, 0.785 mmol, 94% purity, 72% yield) as brownish solid.

LCMS: (ES+) m/z (M+H)⁺=541.2, Rt=3.61 min

### Step 2: 5-(3-bromophenyl)-3-fluoro-4-iodo-5,8,8-trimethyl-5,8,9,10-tetrahydrobenzo[b][1,8]naphthyridin-6(7H)-one (4)

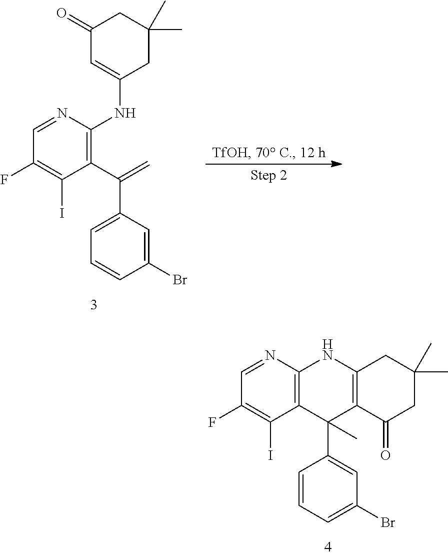 [SMILES: C=C(c1cccc(Br)c1)c1c(NC2=CC(=O)CC(C)(C)C2)ncc(F)c1I ; CC1(C)CC(=O)C2=C(C1)Nc1ncc(F)c(I)c1C2(C)c1cccc(Br)c1]

TfOH (6 mL) was added drop wise to 3-[[3-[1-(3-bromophenyl)vinyl]-5-fluoro-4-iodo-2-pyridyl]amino]-5,5-dimethyl-cyclohex-2-en-1-one (3) (425 mg, 0.785 mmol, 1.00 eq) dropwise at 0° C. Reaction mixture was stirred at 70° C. for 12 h. The reaction mixture was quenched by aqueous NaHCO₃ solution and extracted by EtOAc (2×50 mL). Combined organic layer was washed with brine. Organic layer was dried over Na2SO4, filtered and concentrated under reduced pressure to get crude product purified by combiflash chromatography (column: SiO2, 12 g) using 13-15% EtOAc in Hexane to provide 5-(3-bromophenyl)-3-fluoro-4-iodo-5,8,8-trimethyl-5,8,9,10-tetrahydrobenzo[b][1,8]naphthyridin-6(7H)-one (4) (350 mg, 0.634 mmol, 98% purity, 80% yield) as off white solid.

LCMS: (ES+) m/z (M+H)+=541.2, Rt=3.77 min

### Step 3: 5-(3-bromophenyl)-3-fluoro-5,8,8-trimethyl-6-oxo-5,6,7,8,9,10-hexahydrobenzo[b][1,8]naphthyridine-4-carbonitrile (5)

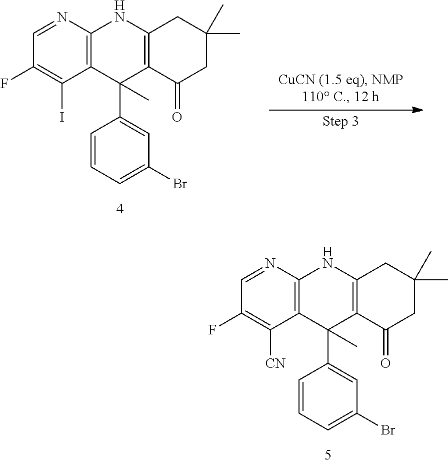 [SMILES: CC1(C)CC(=O)C2=C(C1)Nc1ncc(F)c(I)c1C2(C)c1cccc(Br)c1 ; CC1(C)CC(=O)C2=C(C1)Nc1ncc(F)c(C#N)c1C2(C)c1cccc(Br)c1]

To a stirred solution of 5-(3-bromophenyl)-3-fluoro-4-iodo-5,8,8-trimethyl-5,8,9,10-tetrahydrobenzo[b][1,8]naphthyridin-6(7H)-one (4) (325 mg, 0.601 mmol, 1.00 eq) in NMP (2.5 mL) taken in a sealed tube was added CuCN (107 mg, 1.20 mmol, 2.00 eq). Reaction mixture was stirred at 110° C. for 12 h. Reaction mixture diluted with water (3 mL) and extracted with EtOAc (2×25 mL). Combined organic layer was washed brine. Organic layer was dried over Na2SO4, filtered and concentrated under reduced pressure to get crude product which was purified by combiflash column chromatography (Column: SiO2, 4 g) using 20-30% EtOAc in Hexane to provide 5-(3-bromophenyl)-3-fluoro-5,8,8-trimethyl-6-oxo-5,6,7,8,9,10-hexahydrobenzo[b][1,8]naphthyridine-4-carbonitrile (5) (160 mg, 0.363 mmol, 95% purity, 61% yield) as light yellowish solid.

LCMS: (ES+) m/z (M+H)⁺=440.2, Rt=1.98 min

### Step 4: 3-fluoro-5,8,8-trimethyl-6-oxo-5-(3-(4,4,5,5-tetramethyl-1,3,2-dioxaborolan-2-yl)phenyl)-5,6,7,8,9,10-hexahydrobenzo[b][1,8]naphthyridine-4-carbonitrile (6)

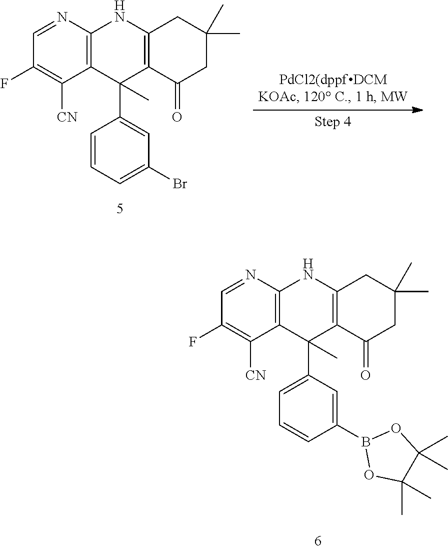 [SMILES: CC1(C)CC(=O)C2=C(C1)Nc1ncc(F)c(C#N)c1C2(C)c1cccc(Br)c1 ; CC1(C)CC(=O)C2=C(C1)Nc1ncc(F)c(C#N)c1C2(C)c1cccc(B2OC(C)(C)C(C)(C)O2)c1]

To a stirred solution of 5-(3-bromophenyl)-3-fluoro-5,8,8-trimethyl-6-oxo-5,6,7,8,9,10-hexahydrobenzo[b][1,8]naphthyridine-4-carbonitrile (5) (50 mg, 0.114 mmol, 1.00 eq) in dry Dioxane (2 mL) taken in a microwave vial were added B2Pin2 (144 mg, 0.568 mmol, 5.00 eq) and dry KOAc (56 mg, 0.568 mmol, 5.00 eq). The reaction mixture was purged with argon for 15 mins. Then Pd(dppf)Cl2·DCM (19 mg, 0.0227 mmol, 0.200 eq) was added to the reaction mixture and irradiated in microwave at 120° C. for 1 h. The reaction mixture was diluted with water (2 mL) and extracted with EtOAc (2×25 ml). Organic layer was dried over Na2SO4, filtered and concentrated under reduced pressure to get crude product which was purified by Prep TLC using 30% EtOAc in hexane to provide 3-fluoro-5,8,8-trimethyl-6-oxo-5-(3-(4,4,5,5-tetramethyl-1,3,2-dioxaborolan-2-yl)phenyl)-5,6,7,8,9,10-hexahydrobenzo[b][1,8]naphthyridine-4-carbonitrile (6) (30 mg, 0.0616 mmol, 60% purity, 54% yield) as brown sticky solid.

LCMS: (ES+) m/z (M+H)⁺=488.2, Rt=2.04 min

### Step 5: 3-fluoro-5,8,8-trimethyl-6-oxo-5-(3-(3-(2,2,2-trifluoroethyl)pyridin-4-yl)phenyl)-5,6,7,8,9,10-hexahydrobenzo[b][1,8]naphthyridine-4-carbonitrile (8)

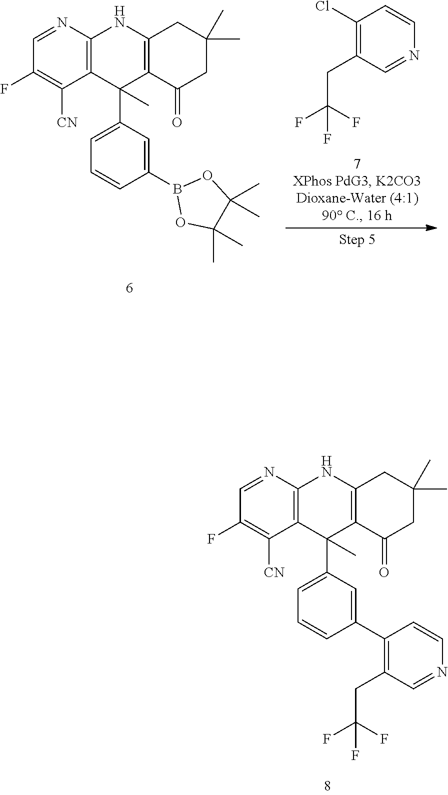 [SMILES: CC1(C)CC(=O)C2=C(C1)Nc1ncc(F)c(C#N)c1C2(C)c1cccc(B2OC(C)(C)C(C)(C)O2)c1 ; FC(F)(F)Cc1cnccc1Cl ; CC1(C)CC(=O)C2=C(C1)Nc1ncc(F)c(C#N)c1C2(C)c1cccc(-c2ccncc2CC(F)(F)F)c1]

To a stirred solution of 3-fluoro-5,8,8-trimethyl-6-oxo-5-(3-(4,4,5,5-tetramethyl-1,3,2-dioxaborolan-2-yl)phenyl)-5,6,7,8,9,10-hexahydrobenzo[b][1,8]naphthyridine-4-carbonitrile (6) (100 mg, 0.205 mmol, 1.00 eq) and 4-chloro-3-(2,2,2-trifluoroethyl)pyridine (7) (80 mg, 0.410 mmol, 2.00 eq) in Dioxane:water (4:1) (2.5 mL) was added K₂CO₃ (85 mg, 0.616 mmol, 3.00 eq). Reaction mixture was purged with argon for 10 mins. Then XPhos Pd G3 (17 mg, 0.0205 mmol, 0.100 eq) was added and reaction mixture was stirred at 90° C. for 16 h. Reaction mixture was diluted with water (2 mL) and extracted with EtOAc (2×25 mL). Combined organic layer was washed with brine. Organic layer was dried over Na2SO4, filtered and concentrated under reduced pressure to get crude product which was purified by combi flash column chromatography (Column: SiO2, 4 g) using 30% EtOAC in Hexane to provide 3-fluoro-5,8,8-trimethyl-6-oxo-5-(3-(3-(2,2,2-trifluoroethyl)pyridin-4-yl)phenyl)-5,6,7,8,9,10-hexahydrobenzo[b][1,8]naphthyridine-4-carbonitrile (8) (45 mg, 0.0865 mmol, 96% purity, 42% yield) as off white solid.

LCMS: (ES+) m/z (M+H)⁺=521.2, Rt=2.21 min

### Step 6: (S)-3-fluoro-5,8,8-trimethyl-6-oxo-5-(3-(3-(2,2,2-trifluoroethyl)pyridin-4-yl)phenyl)-5,6,7,8,9,10-hexahydrobenzo[b][1,8]naphthyridine-4-carbonitrile (8A) & (R)-3-fluoro-5,8,8-trimethyl-6-oxo-5-(3-(3-(2,2,2-trifluoroethyl)pyridin-4-yl)phenyl)-5,6,7,8,9,10-hexahydrobenzo[b][1,8]naphthyridine-4-carbonitrile (8B)

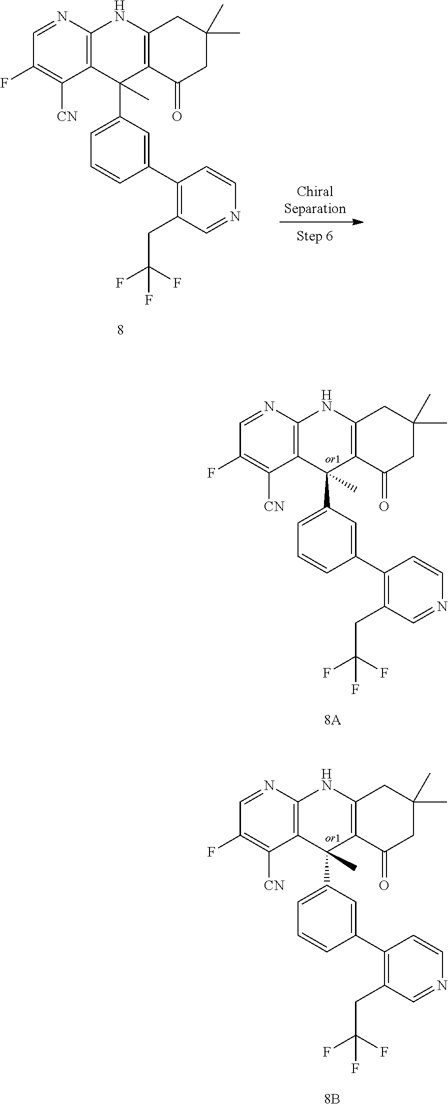 [SMILES: CC1(C)CC(=O)C2=C(C1)Nc1ncc(F)c(C#N)c1C2(C)c1cccc(-c2ccncc2CC(F)(F)F)c1 ; CC1(C)CC(=O)C2=C(C1)Nc1ncc(F)c(C#N)c1[C@]2(C)c1cccc(-c2ccncc2CC(F)(F)F)c1 ; CC1(C)CC(=O)C2=C(C1)Nc1ncc(F)c(C#N)c1[C@@]2(C)c1cccc(-c2ccncc2CC(F)(F)F)c1]

The two enantiomers were separated by chiral preparatory SFC (Column: Chiralpak-IG (30.0 mm×250 mm), 5μ Flow: 100 mL/min Mobile Phase: 75% CO₂+25% (MeOH), ABPR: 100 bars; Temp: 35° C.; UV: 230 nm; diluent: Methanol & DCM to give:

Peak 1: (S)-3-fluoro-5,8,8-trimethyl-6-oxo-5-(3-(3-(2,2,2-trifluoroethyl)pyridin-4-yl)phenyl)-5,6,7,8,9,10-hexahydrobenzo[b][1,8]naphthyridine-4-carbonitrile (8A) (15.94 mg, 99.82% purity, 23% yield) as light yellow solid.

LCMS: Rt=2.14 min., (ES+) m/z (M+H)⁺=521.2

HPLC: (purity: 99.82%)

1H NMR (400 MHz, DMSO-d6) δ 10.26 (s, 1H), 8.63 (s, 1H), 8.57 (d, J=5.2 Hz, 1H), 8.43 (s, 1H), 7.45-7.36 (m, 3H), 7.27 (d, J=4.92 Hz, 1H), 7.11 (d, J=7.28 Hz, 1H), 3.74-3.56 (m, 2H), 2.43 (s, 2H), 2.12 (s, 3H), 2.02-1.89 (m, 2H), 0.99 (s, 3H), 0.91 (s, 3H).

[α]²⁵=+254.702, c=0.1221, CH3CN.

Chiral purity (ee %): 100

Peak 2: (R)-3-fluoro-5,8,8-trimethyl-6-oxo-5-(3-(3-(2,2,2-trifluoroethyl)pyridin-4-yl)phenyl)-5,6,7,8,9,10-hexahydrobenzo[b][1,8]naphthyridine-4-carbonitrile (8B) (17.25 mg, 98% purity, 24% yield) as light yellow solid.

LCMS: Rt=2.14 min., (ES+) m/z (M+H)+=521.2

HPLC: (purity: 98.00%)

1H NMR (400 MHz, DMSO-d6) δ 10.26 (s, 1H), 8.63 (s, 1H), 8.57 (d, J=5.2 Hz, 1H), 8.43 (s, 1H), 7.45-7.36 (m, 3H), 7.27 (d, J=4.92 Hz, 1H), 7.11 (d, J=7.28 Hz, 1H), 3.74-3.56 (m, 2H), 2.43 (s, 2H), 2.12 (s, 3H), 2.02-1.89 (m, 2H), 0.99 (s, 3H), 0.91 (s, 3H).

[α]²⁵=−224.113, c=0.1218, CH3CN.

Chiral purity (ee %): 99.69

### Example 3.18. (5S,9S)-9-fluoro-5,8,8-trimethyl-5-[3-[3-(2,2,2-trifluoroethyl)-4-pyridyl]phenyl]-9,10-dihydro-71H-benzo[b][1,8]naphthyridin-6-one (9A), (5R,9R)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9B), (5S,9R)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9C) & (5R,9S)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9D)

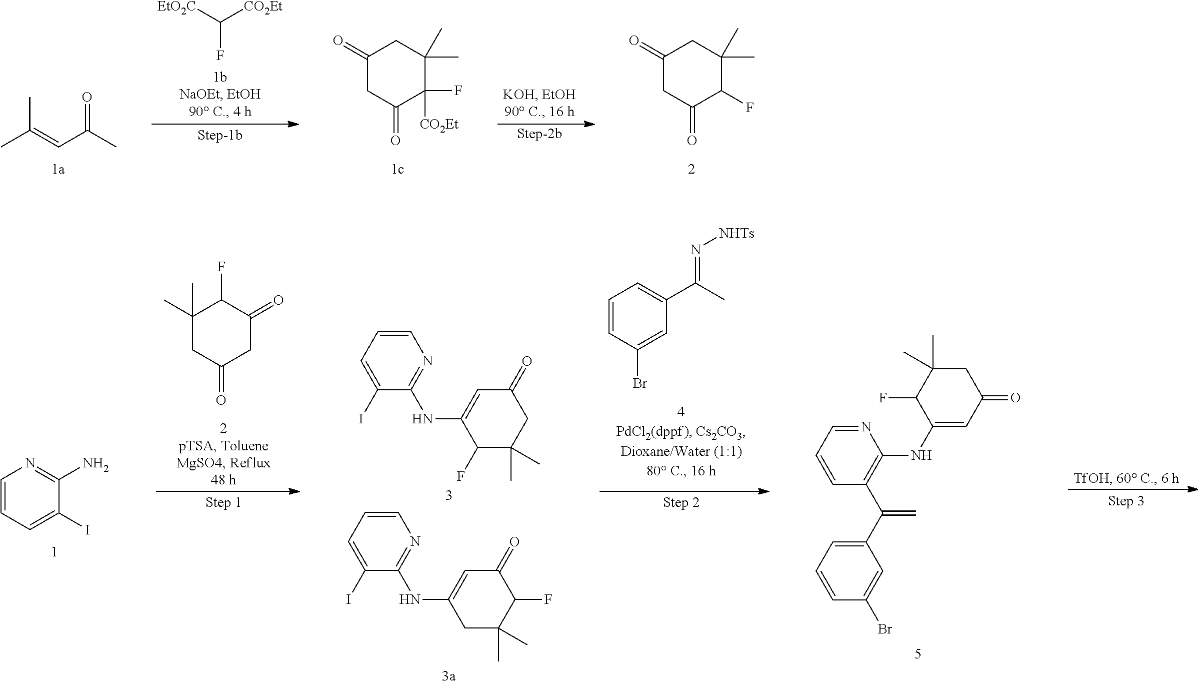

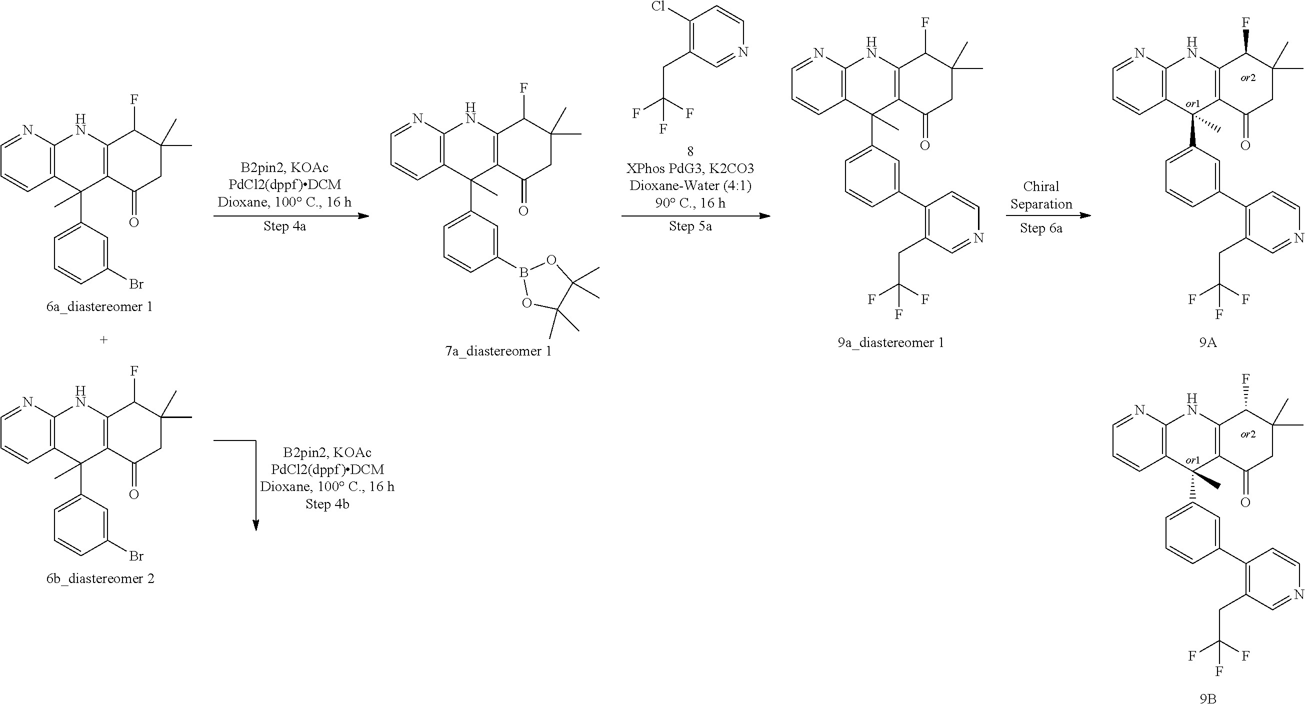

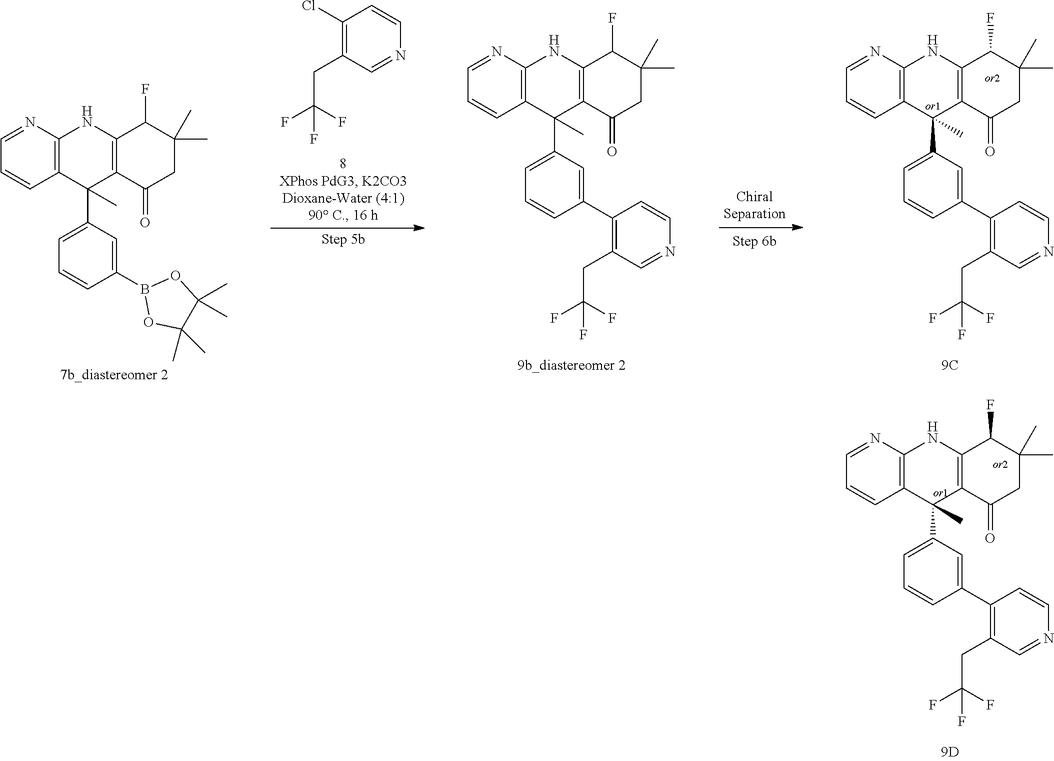 [SMILES: CC1(c2cccc(B3OC(C)(C)C(C)(C)O3)c2)C2=C(Nc3ncccc31)C(F)C(C)(C)CC2=O ; FC(F)(F)Cc1cnccc1Cl ; CC1(c2cccc(-c3ccncc3CC(F)(F)F)c2)C2=C(Nc3ncccc31)C(F)C(C)(C)CC2=O ; CC1(C)CC(=O)C2=C(Nc3ncccc3[C@]2(C)c2cccc(-c3ccncc3CC(F)(F)F)c2)[C@@H]1F ; CC1(C)CC(=O)C2=C(Nc3ncccc3[C@@]2(C)c2cccc(-c3ccncc3CC(F)(F)F)c2)[C@H]1F]

### Step 1: 4-fluoro-3-((3-iodopyridin-2-yl)amino)-5,5-dimethylcyclohex-2-en-1-one (3) & 6-fluoro-3-((3-iodopyridin-2-yl)amino)-5,5-dimethylcyclohex-2-en-1-one (3a)

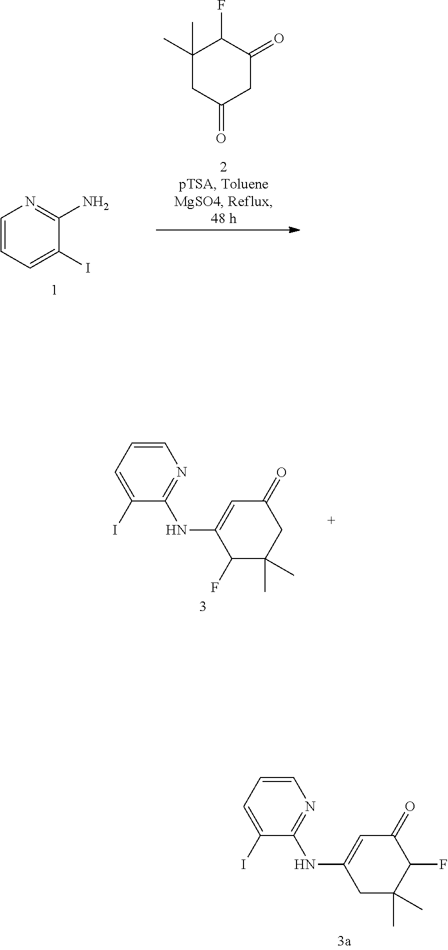 [SMILES: CC1(C)CC(=O)CC(=O)C1F ; Nc1ncccc1I ; CC1(C)CC(=O)C=C(Nc2ncccc2I)C1F ; CC1(C)CC(Nc2ncccc2I)=CC(=O)C1F]

To a stirred solution of 3-iodopyridin-2-amine (1) (3.00 g, 13.6 mmol, 1.00 eq) in Toluene (75 ml) were added 4-fluoro-5,5-dimethyl-cyclohexane-1,3-dione (2) (3.23 g, 20.5 mmol, 1.50 eq), PTSA (518 mg, 2.73 mmol, 0.200 eq) and MgSO4 (6545 mg, 54.5 mmol, 4.00 eq). Reaction mixture was stirred at 120° C. for 48 h. Reaction mixture was concentrated under reduced pressure to get crude product which was purified by combi flash column chromatography (Column: SiO2, 40 g) using 20% EtOAc in hexane to provide 4-fluoro-3-((3-iodopyridin-2-yl)amino)-5,5-dimethylcyclohex-2-en-1-one (3) (500 mg, 1.24 mmol, 90% purity, 9% yield) as brown solid and 6-fluoro-3-((3-iodopyridin-2-yl)amino)-5,5-dimethylcyclohex-2-en-1-one (3a) (1 g, 2.49 mmol, 90% purity, 18% yield) eluted in 70% EtOAc in hexane as brown solid.

LCMS: (ES⁺) m/z (M+H)⁺=361.0, Rt=2.14 min

LCMS: (ES⁺) m/z (M+H)⁺=361.0, Rt=1.65 min

Note: 4-fluoro-3-((3-iodopyridin-2-yl)amino)-5,5-dimethylcyclohex-2-en-1-one (500 mg, 1.24 mmol, 90% purity, 9% yield) (3) was forwarded for synthesis of GF-228-A, GF-228-B, GF-228-C & GF-228-D & 6-fluoro-3-((3-iodopyridin-2-yl)amino)-5,5-dimethylcyclohex-2-en-1-one (3a) was forwarded for synthesis of GF-228-A_regio, GF-228-B_regio, GF-228-C_regio & GF-228-D_regio.

### Step 2: 3-((3-(1-(3-bromophenyl) vinyl) pyridin-2-yl)amino)-4-fluoro-5,5-dimethylcyclohex-2-en-1-one. (5)

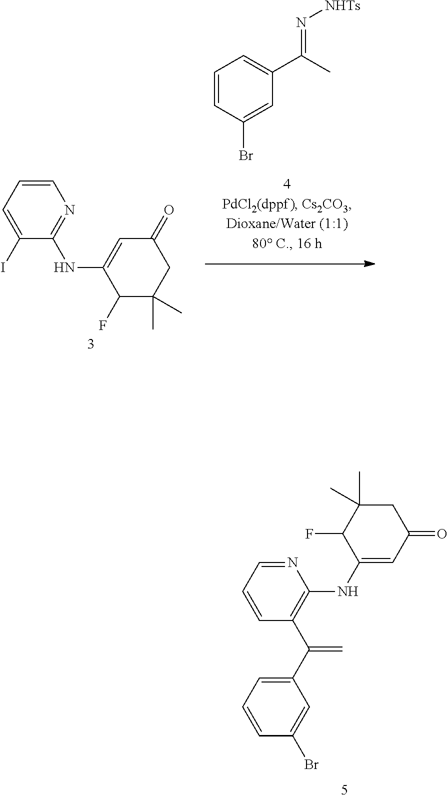

To a stirred solution of 4-fluoro-3-[(3-iodo-2-pyridyl)amino]-5,5-dimethyl-cyclohex-2-en-1-one (3) (1.00 g, 2.78 mmol, 1.00 eq) in Dioxane:water (1:1) (15 mL) were added N-(l-(3-bromophenyl) ethylidene)-4-methylbenzenesulfonohydrazide (4) (2.03 g, 5.55 mmol, 2.00 eq) and Cs2CO3 (2.7 mg, 8.33 mmol, 3.00 eq). The reaction mixture was purged with argon for 10 mins, Then Pd(dppf)Cl2·DCM (227 mg, 0.278 mmol, 0.1000 eq) was added. Reaction mixture was stirred at 80° C. for 16 h. Reaction mixture was diluted with water (5 mL) and extracted with EtOAc (3×100 mL). Combined organic layer was washed with brine. Organic layer was dried over Na2SO4, filtered and concentrated under reduced pressure to get crude product which was purified by combi flash column chromatography (Column: SiO2, 12 g) using 30-40% EtOAc in hexane to provide 3-[[3-[1-(3-bromophenyl)vinyl]-2-pyridyl]amino]-4-fluoro-5,5-dimethyl-cyclohex-2-en-1-one (5) (250 mg, 0.542 mmol, 90.08% purity, 20% yield)

LCMS: (ES⁺) m/z (M+H)⁺=415.1, Rt=2.03 min

### Step 3: 5-(3-bromophenyl)-9-fluoro-5,8,8-trimethyl-5,8,9,10-tetrahydrobenzo[b][1,8] naphthyridin-6(7H)-one (6a_diasteromer 1) & 5-(3-bromophenyl)-9-fluoro-5,8,8-trimethyl-5,8,9,10-tetrahydrobenzo[b][1,8]naphthyridin-6(7H)-one (6b_diasteromer 2)

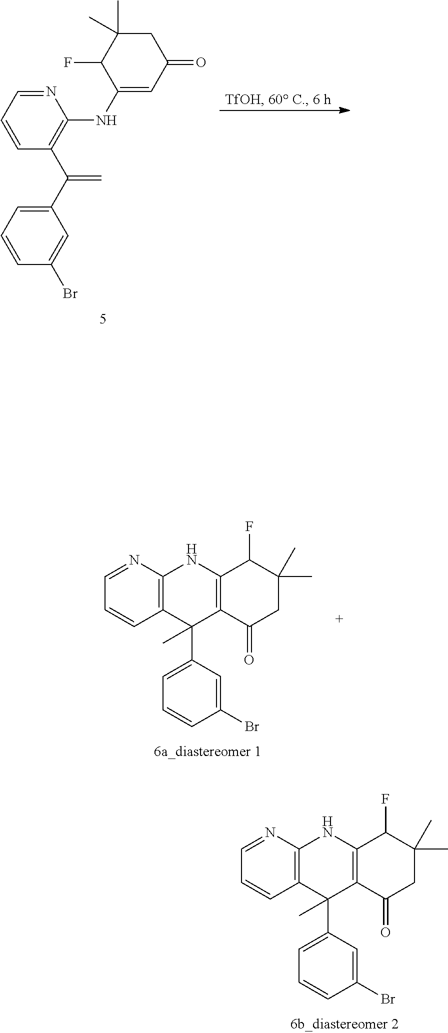 [SMILES: C=C(c1cccc(Br)c1)c1cccnc1NC1=CC(=O)CC(C)(C)C1F ; CC1(c2cccc(Br)c2)C2=C(Nc3ncccc31)C(F)C(C)(C)CC2=O ; CC1(c2cccc(Br)c2)C2=C(Nc3ncccc31)C(F)C(C)(C)CC2=O]

To a stirred solution of 3-[[3-[1-(3-bromophenyl) vinyl]-2-pyridyl]amino]-4-fluoro-5,5-dimethyl-cyclohex-2-en-1-one (5) (400 mg, 0.963 mmol, 1.00 eq) was added TfOH (5 ML) at 00 C. Reaction mixture was stirred at 60° C. for 6 h. Reaction mixture was quenched with aqueous NaHCO₃ solution and extracted with EtOAc (3×50 mL). Combined organic layer was dried over Na2SO4, filtered and concentrated under reduced pressure to get crude product which was purified by combi flash column chromatography (Column: SiO2, 12 g) using 15% EtOAc in hexane to provide (6a_diastereomer 1) 5-(3-bromophenyl)-9-fluoro-5,8,8-trimethyl-5,8,9,10-tetrahydrobenzo[b][1,8]naphthyridin-6(7H)-one (100 mg, 0.24 mmol, 90% purity, 25% yield) as off white solid and (6b_diastereomer 2) 5-(3-bromophenyl)-9-fluoro-5,8,8-trimethyl-5,8,9,10-tetrahydrobenzo[b][1,8]naphthyridin-6(7H)-one (150 mg, 0.36 mmol, 98.17% purity, 38% yield) as off white solid eluted in 25% EtOAc in hexane.

LCMS: (ES+) m/z (M+H)⁺=415.1, Rt=2.06 min (6a_diastereomer 1).

LCMS: (ES+) m/z (M+H)⁺=415.2, Rt=2.28 min (6b_diastereomer 2).

### Step 4a: 9-fluoro-5,8,8-trimethyl-5-(3-(4,4,5,5-tetramethyl-1,3,2-dioxaborolan-2-yl) phenyl)-5,8,9,10-tetrahydrobenzo[b][1,8]naphthyridin-6(7H)-one. (7a_diastereomer 1)

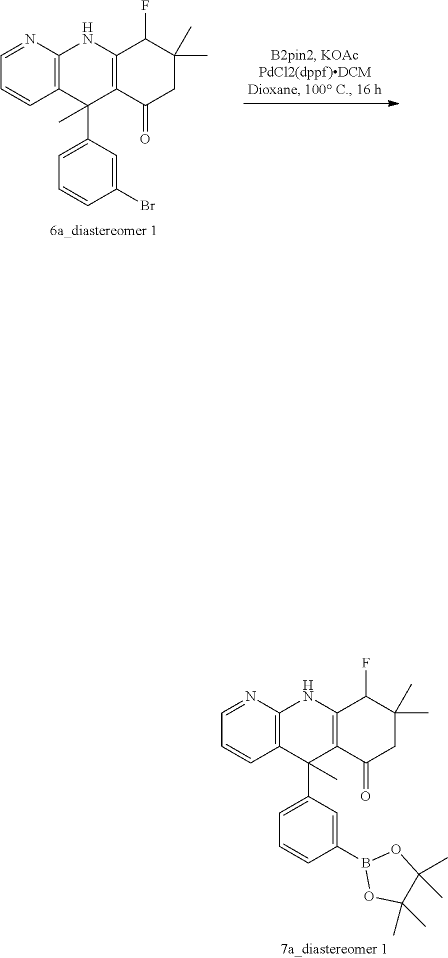 [SMILES: CC1(c2cccc(Br)c2)C2=C(Nc3ncccc31)C(F)C(C)(C)CC2=O ; CC1(c2cccc(B3OC(C)(C)C(C)(C)O3)c2)C2=C(Nc3ncccc31)C(F)C(C)(C)CC2=O]

To a stirred solution of (6a_Diastereomer 1) 5-(3-bromophenyl)-9-fluoro-5,8,8-trimethyl-9,10-dihydro-7H-benzo[b][1,8] naphthyridin-6-one (200 mg, 0.482 mmol, 1.00 eq) in dry Dioxane (8 mL) were added B2Pin2 (609 mg, 2.41 mmol, 5.00 eq) and dry KOAc (236 mg, 2.41 mmol, 5.00 eq). Reaction mixture was degassed for 15 mins, Then Pd(dppf)Cl₂·DCM (79 mg, 0.0963 mmol, 0.200 eq) was added for 16 h at 100° C. The reaction mixture was diluted with water (4 mL) and extracted with EtOAc (3×25 mL). Combined organic layer was dried over Na2SO4, filtered and concentrated under reduced pressure to provide 9-fluoro-5,8,8-trimethyl-5-(3-(4,4,5,5-tetramethyl-1,3,2-dioxaborolan-2-yl) phenyl)-5,8,9,10-tetrahydrobenzo[b][1,8]naphthyridin-6(7H)-one (7a_diastereomer 1) (200 mg, 0.43 mmol, 90% yield) and compound was used for the next step without further purification.

LCMS: (ES⁺) m/z (M+H)⁺=463.3, Rt=2.06 min (7a_Diastereomer 1).

### Step 4b: 9-fluoro-5,8,8-trimethyl-5-(3-(4,4,5,5-tetramethyl-1,3,2-dioxaborolan-2-yl) phenyl)-5,8,9,10-tetrahydrobenzo[b] [1,8]naphthyridin-6(7H)-one (7b_diastereomer 2)

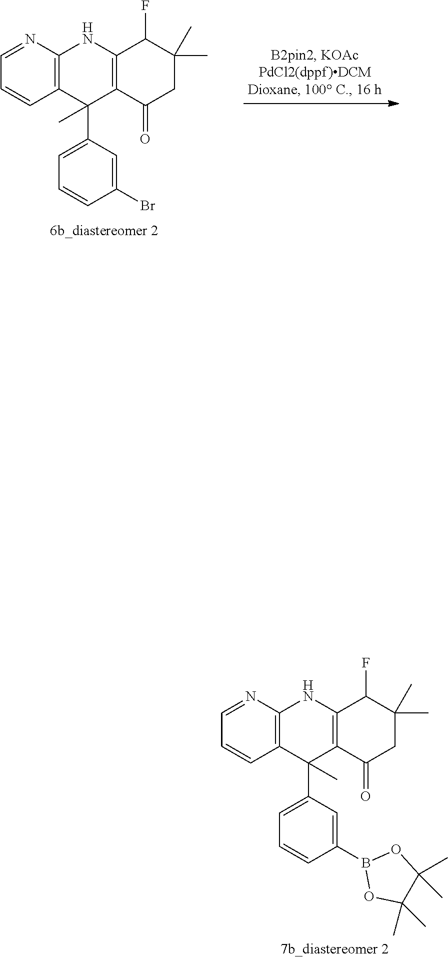 [SMILES: CC1(c2cccc(Br)c2)C2=C(Nc3ncccc31)C(F)C(C)(C)CC2=O ; CC1(c2cccc(B3OC(C)(C)C(C)(C)O3)c2)C2=C(Nc3ncccc31)C(F)C(C)(C)CC2=O]

To a stirred solution of (6b_diastereomer 2) 5-(3-bromophenyl)-9-fluoro-5,8,8-trimethyl-9,10-dihydro-7H-benzo[b][1,8]naphthyridin-6-one (300 mg, 0.722 mmol, 1.00 eq) in dry Dioxane (11 mL) were added B2Pin2 (914 mg, 3.61 mmol, 5.00 eq) and dry KOAC (354 mg, 3.61 mmol, 5.00 eq). Reaction mixture was degassed for 15 mins, was added for 16 h at 100° C. Reaction mixture was diluted with water (4 mL) and extracted with EtOAc (3×25 mL). Combined organic layer was dried over Na2SO4, filtered and concentrated under reduced pressure to provide 9-fluoro-5,8,8-trimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one (7b_diastereomer 2) (300 mg, 0.65 mmol, 90% yield) and compound was used for the next step without further purification.

LCMS: (ES+) m/z (M+H)⁺=463.3, Rt=2.06 min (7b_diastereomer 2).

### Step 5a: 9-fluoro-5,8,8-trimethyl-5-(3-(2,2,2-trifluoroethyl) pyridin-4-yl) phenyl)-5,8,9,10-tetrahydrobenzo[b][1,8] naphthyridin-6(7H)-one (9a_diastereomer 1)

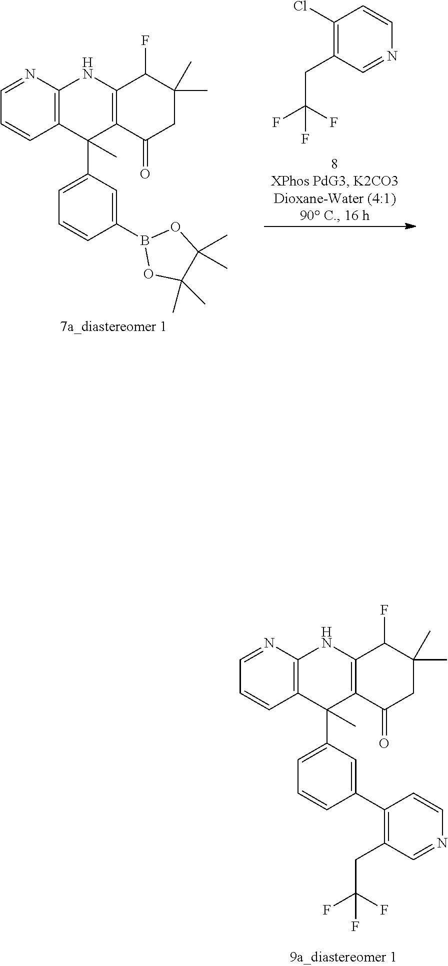 [SMILES: CC1(c2cccc(B3OC(C)(C)C(C)(C)O3)c2)C2=C(Nc3ncccc31)C(F)C(C)(C)CC2=O ; FC(F)(F)Cc1cnccc1Cl ; CC1(c2cccc(-c3ccncc3CC(F)(F)F)c2)C2=C(Nc3ncccc31)C(F)C(C)(C)CC2=O]

To a stirred solution of (7a_diastereomer 1) 9-fluoro-5,8,8-trimethyl-5-[3-(4,4,5,5-tetramethyl-1,3,2-dioxaborolan-2-yl)phenyl]-9,10-dihydro-7H-benzo[b][1,8]naphthyridin-6-one (200 mg, 0.433 mmol, 1.00 eq) in Dioxane:water (4:1) (10 mL) were added 4-chloro-3-(2,2,2-trifluoroethyl)pyridine (169 mg, 0.865 mmol, 2.00 eq) and K₂CO₃ (179 mg, 1.30 mmol, 3.00 eq). Reaction mixture was purged with argon for 10 mins. Then XPhos Pd G3 (37 mg, 0.0433 mmol, 0.100 eq) was added and reaction mixture was stirred at 900 C for 16 h. Reaction mixture was diluted with water (5 mL) and extracted with EtOAC (2×50 mL). Combined organic layer was dried over Na2SO4, filtered and concentrated under reduced pressure to get crude product which was purified by Prep TLC using 30% EtOAc in hexane to provide 9-fluoro-5,8,8-trimethyl-5-(3-(3-(2,2,2-trifluoroethyl) pyridin-4-yl) phenyl)-5,8,9,10-tetrahydrobenzo[b][1,8] naphthyridin-6(7H)-one (70 mg, 0.14 mmol, 97.42% purity, 32% purity) (9a_diastereomer 1) as off white solid.

LCMS: (ES+) m/z (M+H)⁺=496.2, Rt=2.86 min (9a_diastereomer 1).

Step 5b: 9-fluoro-5,8,8-trimethyl-5-(3-(2,2,2-trifluoroethyl) pyridin-4-yl) phenyl)-5,8,9,10-tetrahydrobenzo[b][1,8] naphthyridin-6(7H)-one (9b_diastereomer 2).

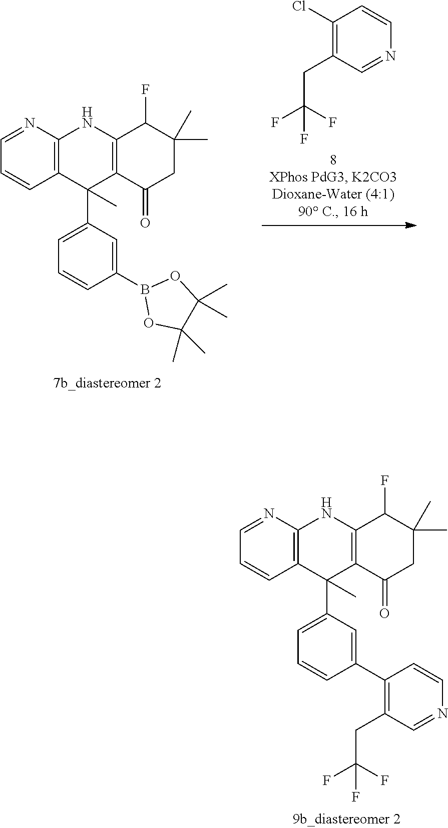 [SMILES: CC1(c2cccc(B3OC(C)(C)C(C)(C)O3)c2)C2=C(Nc3ncccc31)C(F)C(C)(C)CC2=O ; FC(F)(F)Cc1cnccc1Cl ; CC1(c2cccc(-c3ccncc3CC(F)(F)F)c2)C2=C(Nc3ncccc31)C(F)C(C)(C)CC2=O]

To a stirred solution of (7b_diastereomer 2) 9-fluoro-5,8,8-trimethyl-5-[3-(4,4,5,5-tetramethyl-1,3,2-dioxaborolan-2-yl)phenyl]-9,10-dihydro-7H-benzo[b][1,8]naphthyridin-6-one (260 mg, 0.562 mmol, 1.00 eq) in Dioxane:water(4:1) (12 mL) were added 4-chloro-3-(2,2,2-trifluoroethyl)pyridine (220 mg, 1.12 mmol, 2.00 eq) and K₂CO₃ (233 mg, 1.69 mmol, 3.00 eq). Reaction mixture was purged with argon for 10 mins. Then XPhos Pd G3 (48 mg, 0.0562 mmol, 0.100 eq) was added and continued the reaction at 90° C. for 16 h. Reaction mixture was diluted with water (5 mL) and extracted with EtOAc (2×50 mL). Combined organic layer was dried over Na2SO4, filtered and concentrated under reduced pressure to get crude product which was purified by Prep TLC using 40% EtOAc in hexane to provide 9-fluoro-5,8,8-trimethyl-5-(3-(3-(2,2,2-trifluoroethyl) pyridin-4-yl) phenyl)-5,8,9,10-tetrahydrobenzo[b][1,8]naphthyridin-6(7H)-one (80 mg, 0.14 mmol, 94.08% purity, 27% purity) (9b_diastereomer 2).

LCMS: (ES+) m/z (M+H)⁺=496.2, Rt=1.87 min (9b_diastereomer 2).

### Step 6a: (5S,9S)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9A) & (5R,9R)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9B)

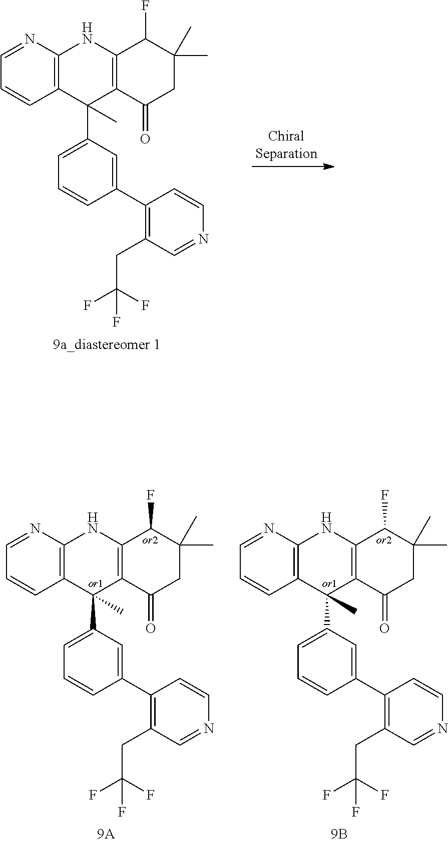

The two enantiomers were separated by chiral preparatory SFC (Column: Reflect (R, R) Whelk_O1 (30.0 mm×250 mm), 5μ Flow: 90 mL/min Mobile Phase: 60% CO₂+40% of CH3CN:THF (1:1), ABPR: 120 bars; Temp: 35° C.; UV: 337 nm; diluent: CH3CN to give:

Peak 1: (5S,9S)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9A), (12 mg, 0.02 mmol, 98.92% purity, 7% yield) as white solid.

LCMS: Rt=2.05 min., (ES+) m/z (M+H)⁺=496.2

HPLC: (purity: 98.92%)

¹H NMR (400 MHz, DMSO-d₆) δ 10.10 (s, 1H), 8.63 (s, 1H), 8.57 (d, J=4.96 Hz, 1H), 8.02 (d, J=3.48 Hz, 1H), 7.41-7.40 (m, 2H), 7.29-7.27 (m, 2H), 7.12-7.08 (m, 2H), 6.86-6.83 (m, 1H), 5.08 (d, J=49.4 Hz, 1H), 3.65-3.57 (m, 2H), 2.27 (d, J=16.4 Hz, 1H), 1.98-1.92 (m, 4H), 1.05 (s, 3H), 0.99 (s, 3H).

[α]²⁵=+107.164, c=0.1316, DCM.

Chiral purity (ee %): 100

Peak 2: (5R,9R)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9B), (12.11 mg, 0.02 mmol, 98.66% purity, 8% yield) as white solid.

LCMS: Rt=2.05 min., (ES⁺) m/z (M+H)⁺=496.2

HPLC: (purity: 98.66%)

¹H NMR (400 MHz, DMSO-d₆) δ 10.10 (s, 1H), 8.63 (s, 1H), 8.57 (d, J=4.96 Hz, 1H), 8.02 (d, J=3.48 Hz, 1H), 7.41-7.40 (m, 2H), 7.29-7.27 (m, 2H), 7.12-7.08 (m, 2H), 6.86-6.83 (m, 1H), 5.08 (d, J=49.4 Hz, 1H), 3.65-3.57 (m, 2H), 2.27 (d, J=16.4 Hz, 1H), 1.98-1.92 (m, 4H), 1.05 (s, 3H), 0.99 (s, 3H).

[α]²⁵=−104.124, c=0.1316, DCM.

Chiral purity (ee %): 99.32

### Step 6b: (5S,9R)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9C) & (5R,9S)-9-fluoro-5,8,8-trimethyl-5-[3-[3-(2,2,2-trifluoroethyl)-4-pyridyl]phenyl]-9,10-dihydro-71H-benzo[b][1,8]naphthyridin-6-one (9D)

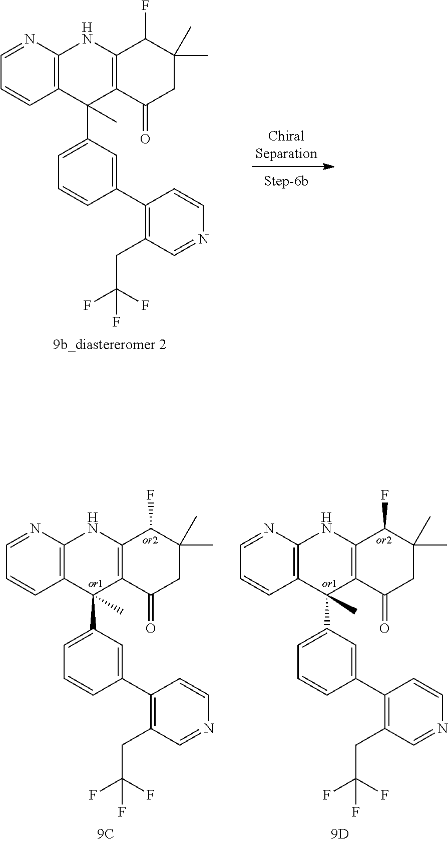

The two enantiomers were separated by chiral preparatory SFC Column: (R, R) Whelk_O1 (30.0 mm×250 mm), 5μ Flow: 110 mL/min Mobile Phase: 70% CO₂+100% of CH3CN, ABPR: 120 bars; Temp: 35° C.; UV: 220 nm; diluent: CH3CN to give:

Peak 1: (5S,9R)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9C), (2.01 mg, 0.01 mmol, 93.92% purity, 1.2% yield) as white solid.

LCMS: Rt=1.99 min., (ES⁺) m/z (M+H)⁺=496.2

HPLC: (purity: 93.92%)

¹H NMR (400 MHz, DMSO-d₆) δ 10.12 (s, 1H), 8.64 (s, 1H), 8.57 (d, J=4.96 Hz, 2H), 8.02 (d, J=3.48 Hz, 1H), 7.42-7.41 (m, 2H), 7.30-7.28 (m, 2H), 7.09 (d, J=6.4 Hz, 2H), 6.85-6.82 (m, 1H), 5.05 (d, J=47.2 Hz, 1H), 3.66-3.55 (m, 2H), 2.22 (d, J=16.4 Hz, 1H), 2.00 (d, J=16.4 Hz, 1H), 1.92 (s, 3H), 1.05 (s, 3H), 0.99 (s, 3H).

[α]²⁵=+102.638, c=0.1325, DCM.

Chiral purity (ee %): 100

Peak 2: (5R,9S)-9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9D) (5.96 mg, 0.01 mmol, 98.52% purity, 4% yield) as white solid.

LCMS: Rt=1.99 min., (ES⁺) m/z (M+H)⁺=496.2

HPLC: (purity: 98.52%)

¹H NMR (400 MHz, DMSO-d₆) δ 10.12 (s, 1H), 8.64 (s, 1H), 8.57 (d, J=4.96 Hz, 2H), 8.02 (d, J=3.48 Hz, 1H), 7.42-7.41 (m, 2H), 7.30-7.28 (m, 2H), 7.09 (d, J=6.4 Hz, 2H), 6.85-6.82 (m, 1H), 5.05 (d, J=47.2 Hz, 1H), 3.66-3.55 (m, 2H), 2.22 (d, J=16.4 Hz, 1H), 2.00 (d, J=16.4 Hz, 1H), 1.92 (s, 3H), 1.05 (s, 3H), 0.99 (s, 3H).

[α]²⁵=−109.175, c=0.1374, DCM.

Chiral purity (ee %): 98.78

### Example 3.19.4-fluoro-5,5-dimethylcyclohexane-1,3-dione (2)

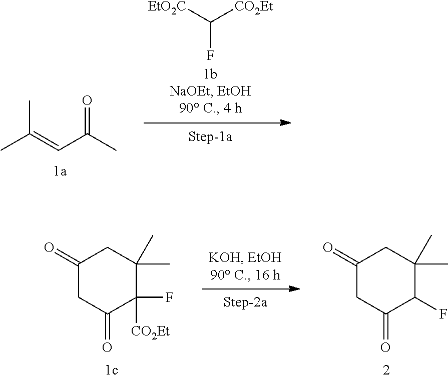

### Step 1a: ethyl 1-fluoro-2,2-dimethyl-4,6-dioxocyclohexane-1-carboxylate (1c)

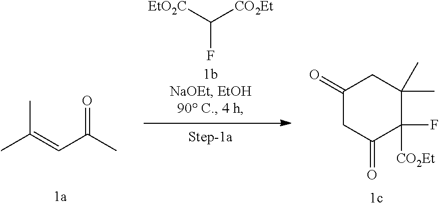

Na Metal (2.73 g, 119 mmol, 1.10 eq) was added portion wise to a stirred solution of EtOH (300 mL). 4-methylpent-3-en-2-one (1a) (11.66 mL, 102 mmol, 0.945 eq) and diethyl 2-fluoropropanedioate (1b) (17 mL, 108 mmol, 1.00 eq) was added to the reaction mixture after Na metal was dissolved. Then reaction mixture was stirred at 90° C. for 4 h. EtOH was removed under reduced pressure to obtain a brownish crude residue which was diluted with water (300 mL) and washed with diethyl ether (2×200 mL), aqueous layer was separated and acidified with aqueous HCl (2N) up to pH=7. The Aqueous layer was extracted with EtOAc (3×250 mL). Combined organic layer was dried over Na2SO4, filtered and concentrated under reduced pressure to get crude product which triturated with Hexane to provide ethyl 1-fluoro-2,2-dimethyl-4,6-dioxo-cyclohexanecarboxylate (1c) (21.00 g, 90.9 mmol, 99.65% purity, 84% yield) as off white solid.

LCMS: (ES⁺) m/z (M+H)⁺=231.2, Rt=1.81 min

### Step 2a: 4-fluoro-5,5-dimethylcyclohexane-1,3-dione (2)

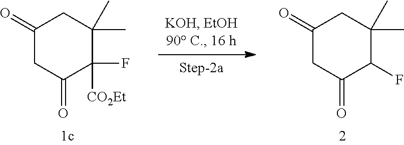 [SMILES: CCOC(=O)C1(F)C(=O)CC(=O)CC1(C)C ; CC1(C)CC(=O)CC(=O)C1F]

To a stirred solution of ethyl 1-fluoro-2,2-dimethyl-4,6-dioxo-cyclohexanecarboxylate (1c) (21.00 g, 91.2 mmol, 1.00 eq) in EtOH (300 mL) was added KOH (20.43 g, 365 mmol, 4.00 eq) nitrogen atmosphere. The resulting mixture was allowed to stir at 90° C. for 16 h. Volatiles were evaporated under reduced pressure to obtain a reddish solid crude which was dissolved in water (100 mL) followed by washed with diethyl ether:hexane (1:1) (2×200 mL). The aqueous layer was collected and acidified with aqueous HCl solution (2N) up to pH=1 at 0° C. The resulting mixture was allowed to stir at RT for 10 mins followed by extraction with EtOAc (3×300 mL). The combined organic layer was dried Na2SO4, filtered and concentrated under reduced pressure to get crude product which was triturated with diethyl ether:hexane (1:1) to provide 4-fluoro-5,5-dimethyl-cyclohexane-1,3-dione (2) (10.00 g, 63.2 mmol, 100% purity, 69% yield) as off white solid.

¹H NMR (400 MHz, DMSO-d₆) δ 11.43 (bs, 1H), 5.20 (s, 1H), 4.74 (d, J=48.72 Hz, 1H), 2.22 (dd, J=17.32 Hz, 17.32 Hz, 1H), 1.07 (s, 3H), 0.94 (s, 3H).

### Example 3.20. (5S,7R)-7-fluoro-5,8,8-trimethyl-5-[3-[3-(2,2,2-trifluoroethyl)-4-pyridyl] phenyl]-9,10-dihydro-7H-benzo[b][1,8]naphthyridin-6-one (9A_regio), (5R,7S)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (93 regio), (5S,7S)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9C_regio) & (5R,7R)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9D_regio)

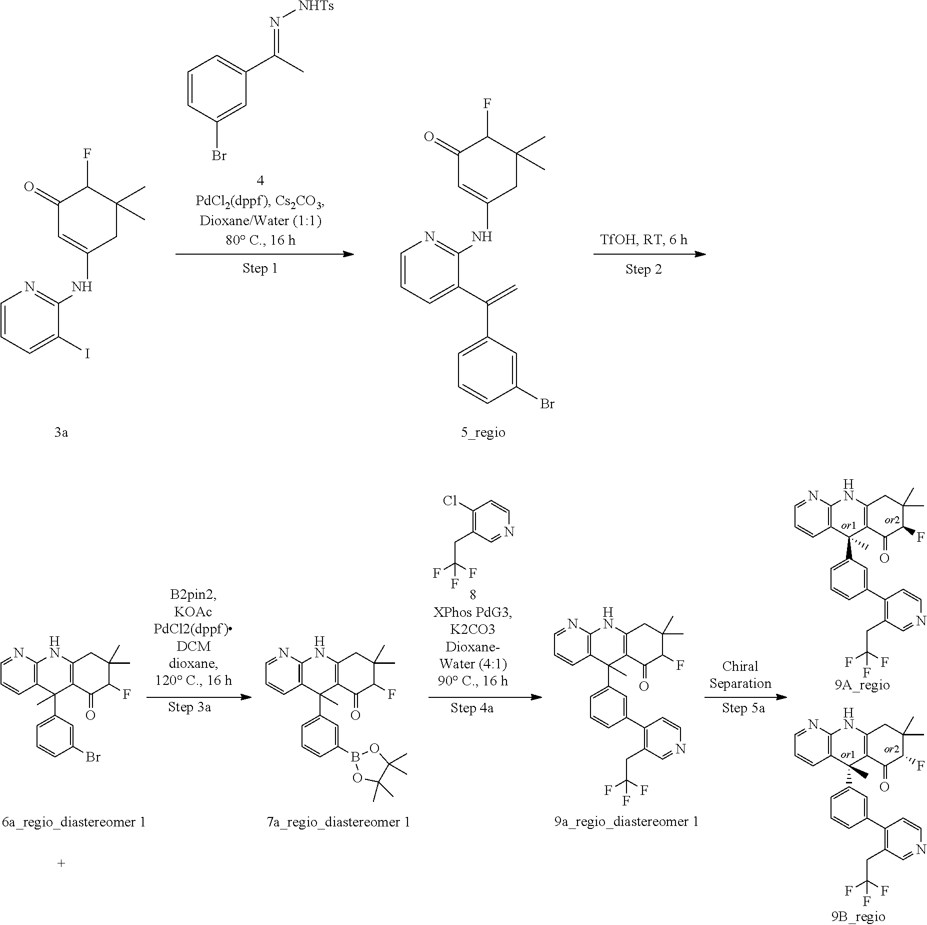

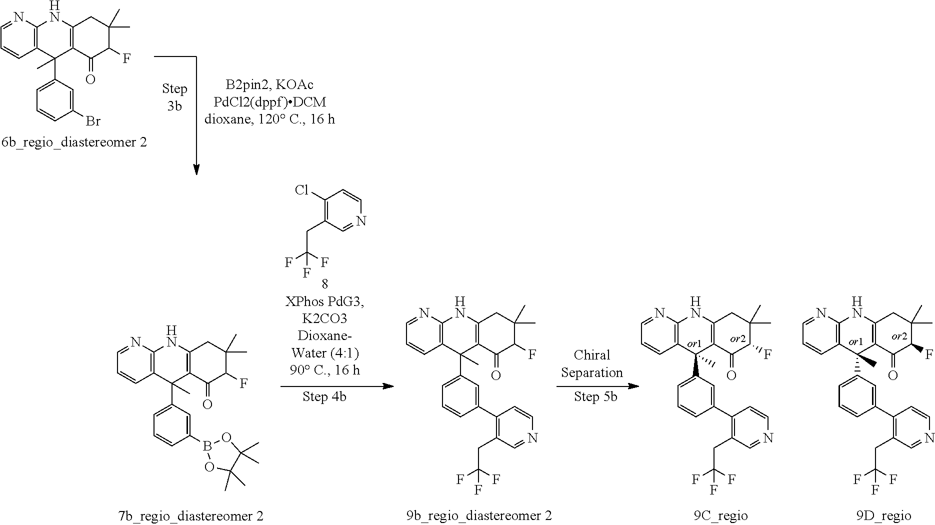 [SMILES: CC1(c2cccc(Br)c2)C2=C(CC(C)(C)C(F)C2=O)Nc2ncccc21 ; FC(F)(F)Cc1cnccc1Cl ; CC1(c2cccc(B3OC(C)(C)C(C)(C)O3)c2)C2=C(CC(C)(C)C(F)C2=O)Nc2ncccc21 ; CC1(c2cccc(-c3ccncc3CC(F)(F)F)c2)C2=C(CC(C)(C)C(F)C2=O)Nc2ncccc21 ; CC1(C)CC2=C(C(=O)[C@H]1F)[C@@](C)(c1cccc(-c3ccncc3CC(F)(F)F)c1)c1cccnc1N2 ; CC1(C)CC2=C(C(=O)[C@@H]1F)[C@](C)(c1cccc(-c3ccncc3CC(F)(F)F)c1)c1cccnc1N2]

### Step 1: 6-fluoro-5,5-dimethyl-3-((3-(1-phenylvinyl) pyridin-2-yl) amino)cyclohex-2-en-1-one (5_regio)

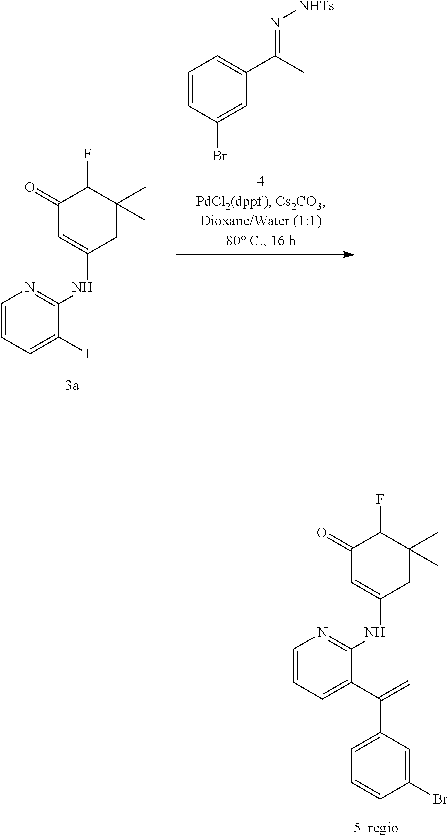

To a stirred solution of 6-fluoro-3-[(3-iodo-2-pyridyl)amino]-5,5-dimethyl-cyclohex-2-en-1-one (3a) (2.20 g, 6.11 mmol, 1.00 eq) in Dioxane:water(1:1) (50 mL) were added N-[(E)-1-(3-bromophenyl)ethylideneamino]-4-methyl-benzenesulfonamide (4) (4.48 g, 12.2 mmol, 2.00 eq) and Cs2CO3 (5.95 g, 18.3 mmol, 3.00 eq). Reaction mixture was purged with argon for 10 mins. Then Pd(dppf)₂Cl2·DCM (498 mg, 0.611 mmol, 0.1000 eq) was added and the reaction was stirred at 80° C. for 16 h. The reaction mixture was diluted water (10 mL) and extracted with EtOAc (3×100 mL). Combined organic layer was washed with brine. Organic layer was dried over Na2SO4, filtered and concentrated under reduced pressure to get crude product which was purified by combi flash column chromatography (Column: SiO2, 40 g) using 50-60% EtOAc in hexane to afford 3-[[3-[1-(3-bromophenyl)vinyl]-2-pyridyl]amino]-6-fluoro-5,5-dimethyl-cyclohex-2-en-1-one (5_regio) (490 mg, 1.06 mmol, 90% purity, 17% yield) as brown solid.

LCMS: (ES+) m/z (M+H)⁺=415.1, Rt=1.84 min

### Step 2: 5-(3-bromophenyl)-7-fluoro-5,8,8-trimethyl-5,8,9,10-tetrahydrobenzo[b][1,8] naphthyridin-6(7H)-one (6a_regio_diastereomer 1) & 5-(3-bromophenyl)-7-fluoro-5,8,8-trimethyl-5,8,9,10-tetrahydrobenzo[b][1,8]naphthyridin-6(7H)-one (6b_regio_diastereomer 2)

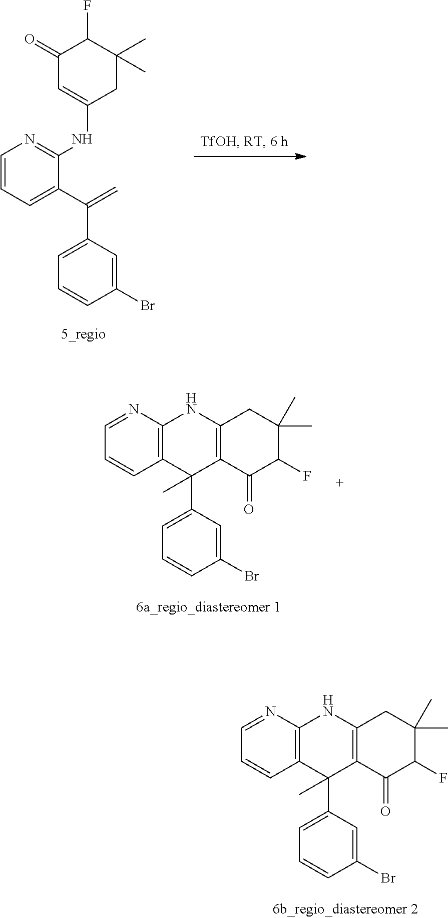 [SMILES: C=C(c1cccc(Br)c1)c1cccnc1NC1=CC(=O)C(F)C(C)(C)C1 ; CC1(c2cccc(Br)c2)C2=C(CC(C)(C)C(F)C2=O)Nc2ncccc21 ; CC1(c2cccc(Br)c2)C2=C(CC(C)(C)C(F)C2=O)Nc2ncccc21]

To a stirred solution of 3-[[3-[l-(3-bromophenyl) vinyl]-2-pyridyl]amino]-6-fluoro-5,5-dimethyl-cyclohex-2-en-1-one (5_regio) (700 mg, 1.69 mmol, 1.00 eq) was added TfOH at 00 C. Reaction mixture was stirred at 60° C. for 4 h. Reaction mixture was quenched with aqueous NaHCO₃ solution and extracted with EtOAc (2×50 mL). Combined organic layer was washed with brine. Organic layer was dried over Na2SO4, filtered and concentrated under reduce pressure to get crude product which was purified by combi flash column chromatography (Column: SiO2, 12 g) using 15% EtOAc in hexane to provide (6a_regio_diastereomer 1) 5-(3-bromophenyl)-7-fluoro-5,8,8-trimethyl-5,8,9,10-tetrahydrobenzo[b][1,8]naphthyridin-6(7H)-one (170 mg, 0.41 mmol, 95% purity, 23% yield) as brown solid and (6b_regio_diastereomer 2) 5-(3-bromophenyl)-7-fluoro-5,8,8-trimethyl-5,8,9,10-tetrahydrobenzo[b][1,8]naphthyridin-6(7H)-one (170 mg, 0.41 mmol, 95% purity, 23% yield) as brown solid eluted in 20% EtOAC in hexane.

(6a_Regio_Diastereomer 1)

¹H NMR (400 MHz, DMSO-d₆) δ 10.09 (s, 1H), 8.01 (d, J=4.6 Hz, 1H), 7.50 (s, 1H), 7.36 (d, J=7.72 Hz, 1H), 7.30 (d, J=8.04 Hz, 1H), 7.21 (t, J=7.88 Hz, 1H), 7.04 (d, J=6.76 Hz, 1H), 6.86-6.83 (m, 1H), 4.58 (d, J=49.28 Hz, 1H), 2.80 (d, J=17.2 Hz, 1H), 2.50-2.48 (d, 1H, merged with DMSO peak), 1.85 (s, 3H), 1.11 (s, 3H), 0.96 (s, 3H).

(6b_Regio_Diastereomer 2)

¹H NMR (400 MHz, DMSO-d6) δ 10.07 (s, 1H), 8.01 (d, J=4.52 Hz, 1H), 7.47 (s, 1H), 7.33-7.28 (m, 2H), 7.24-7.21 (m, 2H), 6.87-6.84 (m, 1H), 4.76 (d, J=49.04 Hz, 1H), 2.73 (d, J=17.2 Hz, 1H), 2.56-2.54 (d, 1H, merged with DMSO peak), 1.89 (s, 3H), 1.15 (s, 3H), 0.96 (s, 3H).

### Step 3a: 7-fluoro-5,8,8-trimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one (7a_regio_diastereomer 1)

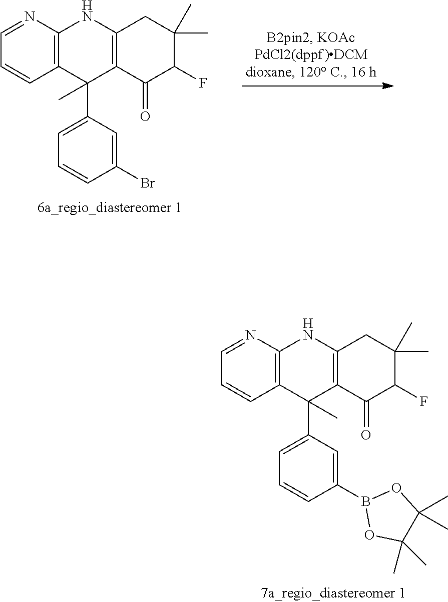 [SMILES: CC1(c2cccc(Br)c2)C2=C(CC(C)(C)C(F)C2=O)Nc2ncccc21 ; CC1(c2cccc(B3OC(C)(C)C(C)(C)O3)c2)C2=C(CC(C)(C)C(F)C2=O)Nc2ncccc21]

To a stirred solution of (6a_regio_diastereomer 1) 5-(3-bromophenyl)-7-fluoro-5,8,8-trimethyl-9,10-dihydro-7H-benzo[b][1,8]naphthyridin-6-one (170 mg, 0.409 mmol, 1.00 eq) in dry Dioxane (4 mL) were added B2Pin2 (520 mg, 2.05 mmol, 5.00 eq) and dry KOAc (120 mg, 1.23 mmol, 3.00 eq). Reaction mixture was purged with argon for 10 mins. Then Pd(dppf)Cl2·DCM (35 mg, 0.0409 mmol, 0.100 eq) was added and reaction was stirred at 100° C. for 16 h. Reaction was mixture was diluted water (3 mL) and extracted with EtOAc (2×50 mL). Organic layer was dried over Na2SO4, filtered and concentrated under reduced pressure to get 7-fluoro-5,8,8-trimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one (7a_regio_diastereomer 1) (170 mg, 0.37 mmol, 90% yield) and compound was used for the next step without further purification.

LCMS: (ES+) m/z (M+H)⁺=463.4, Rt=2.30 min (7a_regio_diastereomer 1).

### Step 3b: 7-fluoro-5,8,8-trimethyl-5-(3-(4,4,5,5-tetramethyl-1,3,2-dioxaborolan-2-yl)phenyl)-5,8,9,10-tetrahydrobenzo[b][1,8]naphthyridin-6(7H)-one (7b_regio_diastereomer 2)

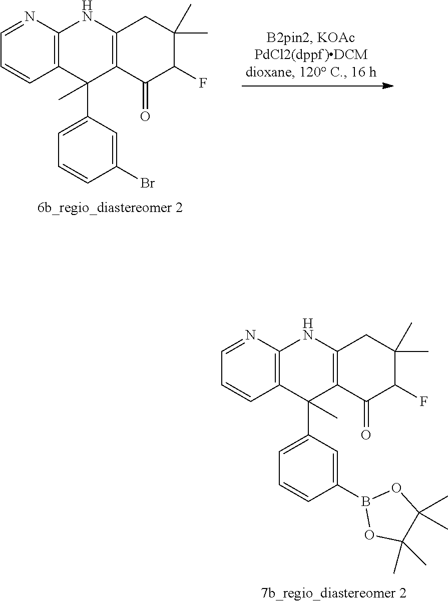 [SMILES: CC1(c2cccc(Br)c2)C2=C(CC(C)(C)C(F)C2=O)Nc2ncccc21 ; CC1(c2cccc(B3OC(C)(C)C(C)(C)O3)c2)C2=C(CC(C)(C)C(F)C2=O)Nc2ncccc21]

To a stirred solution of (6b_diastereomer 2) 5-(3-bromophenyl)-7-fluoro-5,8,8-trimethyl-9,10-dihydro-7H-benzo[b][1,8]naphthyridin-6-one (170 mg, 0.409 mmol, 1.00 eq) in dry Dioxane (4 mL) were added B2Pin2 (520 mg, 2.05 mmol, 5.00 eq) and dry KOAc (120 mg, 1.23 mmol, 3.00 eq). Reaction mixture was purged with argon for 10 mins. Then Pd(dppf)₂Cl2·DCM (35 mg, 0.0409 mmol, 0.100 eq) was added and reaction was stirred at 100° C. for 16 h. Reaction was mixture was diluted water (3 mL) and extracted with EtOAc (2×50 mL). Organic layer was dried over Na2SO4, filtered and concentrated under reduced pressure to get 7-fluoro-5,8,8-trimethyl-5-(3-(4,4,5,5-tetramethyl-1,3,2-dioxaborolan-2-yl)phenyl)-5,8,9,10-tetrahydrobenzo[b][1,8] naphthyridin-6(7H)-one (7b_regio_diastereomer 2) (170 mg, 0.37 mmol, 90% yield) and compound was used for the next step without further purification.

LCMS: (ES+) m/z (M+H)+=463.4, Rt=2.30 min (7b_regio_diastereomer 2).

### Step 4: 7-fluoro-5,8,8-trimethyl-5-(3-(2,2,2-trifluoroethyl) pyridin-4-yl) phenyl)-5,8,9,10-tetrahydrobenzo[b][1,8]naphthyridin-6(7H)-one (9a_regio_diastereomer 1)

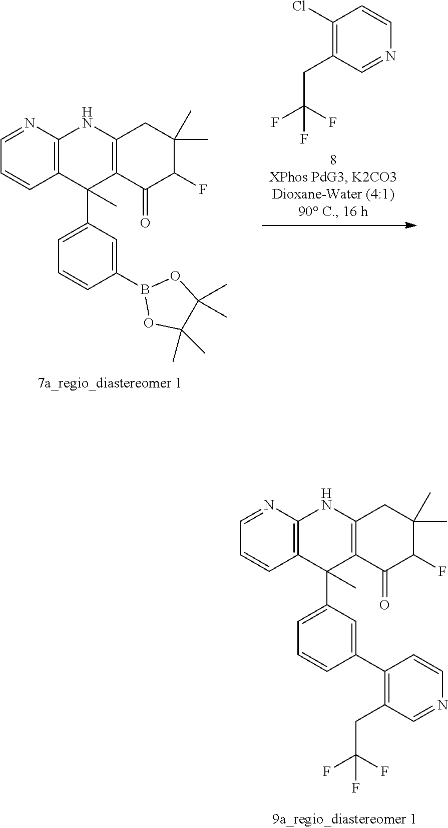 [SMILES: CC1(c2cccc(B3OC(C)(C)C(C)(C)O3)c2)C2=C(CC(C)(C)C(F)C2=O)Nc2ncccc21 ; FC(F)(F)Cc1cnccc1Cl ; CC1(c2cccc(-c3ccncc3CC(F)(F)F)c2)C2=C(CC(C)(C)C(F)C2=O)Nc2ncccc21]

To a stirred solution of (7a_regio_diastereomer 1) 9-fluoro-5,8,8-trimethyl-5-[3-(4,4,5,5-tetramethyl-1,3,2-dioxaborolan-2-yl)phenyl]-9,10-dihydro-7H-benzo[b][1,8]naphthyridin-6-one (170 mg, 0.368 mmol, 1.00 eq) in Dioxane:water (4:1) (10 mL) were added 4-chloro-3-(2,2,2-trifluoroethyl)pyridine (144 mg, 0.735 mmol, 2.00 eq) and K2CO3 (152 mg, 1.10 mmol, 3.00 eq). Reaction mixture was purged with argon for 10 mins. Then XPhos Pd G3 (31 mg, 0.0368 mmol, 0.100 eq) was added and the reaction was stirred at 90° C. for 16 h. Reaction mixture was diluted with water (4 mL) and extracted with EtOAc (2×50 mL). Combined organic layer was washed with brine. Organic layer was dried over Na2SO4, filtered and concentrated under reduced pressure to get crude product which was purified by combi flash column chromatography (Column: SiO2, 4 g) using 50% EtOAc in hexane to provide 9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one, (65 mg, 0.126 mmol, 96.06% purity, 34% yield) (9a_regio_diastereomer 1) as off white solid.

LCMS: (ES+) m/z (M+H)+=496.2, Rt=5.02 min (9a_diastereomer 1).

### Step 4b: 7-fluoro-5,8,8-trimethyl-5-(3-(2,2,2-trifluoroethyl) pyridin-4-yl) phenyl)-5,8,9,10-tetrahydrobenzo[b][1,8]naphthyridin-6(7H)-one (9b_regio_diastereomer 2)

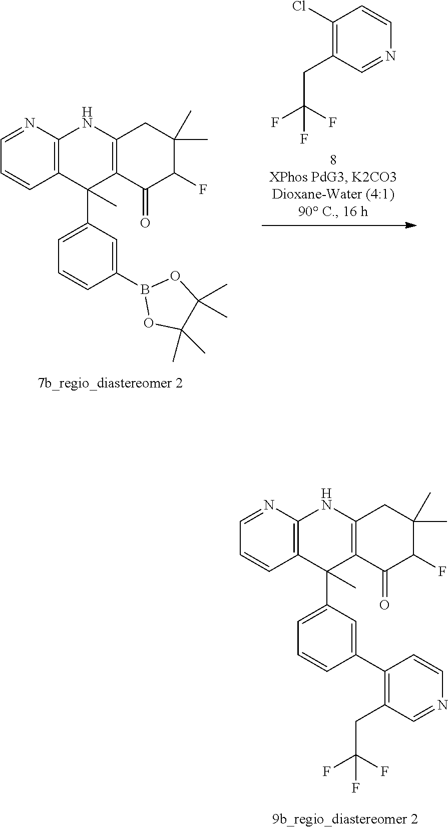 [SMILES: CC1(c2cccc(B3OC(C)(C)C(C)(C)O3)c2)C2=C(CC(C)(C)C(F)C2=O)Nc2ncccc21 ; FC(F)(F)Cc1cnccc1Cl ; CC1(c2cccc(-c3ccncc3CC(F)(F)F)c2)C2=C(CC(C)(C)C(F)C2=O)Nc2ncccc21]

To a stirred solution of (7b_diastereomer 2) 9-fluoro-5,8,8-trimethyl-5-[3-(4,4,5,5-tetramethyl-1,3,2-dioxaborolan-2-yl)phenyl]-9,10-dihydro-7H-benzo[b][1,8]naphthyridin-6-one (170 mg, 0.368 mmol, 1.00 eq) in Dioxane:water(4:1) (10 mL) were added 4-chloro-3-(2,2,2-trifluoroethyl)pyridine (144 mg, 0.735 mmol, 2.00 eq) and K2CO3 (152 mg, 1.10 mmol, 3.00 eq). Reaction mixture was purged with argon for 10 mins. Then XPhos Pd G3 (31 mg, 0.0368 mmol, 0.100 eq) was added and the reaction was stirred at 90° C. for 16 h. Reaction mixture was diluted with water (4 mL) and extracted with EtOAc (2×50 mL). Combined organic layer was washed with brine. Organic layer was dried over Na2SO4, filtered and concentrated under reduced pressure to get crude product which was purified by combi flash column chromatography (Column: SiO2, 4 g) using 50% EtOAc in hexane to provide 9-fluoro-5,8,8-trimethyl-5-[3-[3-(2,2,2-trifluoroethyl)-4-pyridyl]phenyl]-9,10-dihydro-7H-benzo[b][1,8]naphthyridin-6-one, (55 mg, 0.0999 mmol, 90% purity, 27% yield) (9b_regio_diastereomer 2) as off white solid.

LCMS: (ES+) m/z (M+H)+=496.2, Rt=1.40 min (9b_regio_diastereomer 2).

### Step 5a: (5S,7R)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9A_regio) & (5R,7S)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (93_regio)

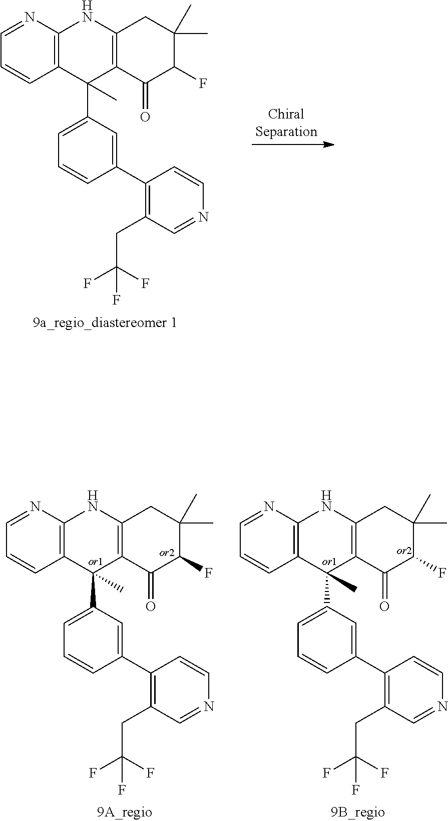

The two enantiomers were separated by chiral preparatory SFC Column: Reflect (R, R) Whelk_O1 (30.0 mm×250 mm), 5μ Flow: 100 mL/min Mobile Phase: 60% CO₂+40% of CH3CN, ABPR: 120 bars; Temp: 35° C.; UV: 230 nm; diluent: CH3CN to give:

Peak 1:(5S,7R)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9A_regio) (13.85 mg, 0.03 mmol, 99.05% purity, 21% yield) as white solid.

LCMS: Rt=1.94 min., (ES⁺) m/z (M+H)⁺=496.2

HPLC: (purity: 99.05%)

¹H NMR (400 MHz, DMSO-d₆) δ 10.04 (s, 1H), 8.62 (s, 1H), 8.57 (s, 1H), 7.99 (s, 1H), 7.47 (d, J=7.2 Hz, 1H), 7.40 (t, J=7.36 Hz, 1H), 7.31 (s, 2H), 7.10 (d, J=7.2 Hz, 1H), 7.03 (d, J=7.16 Hz, 1H), 6.82 (s, 1H), 4.54 (d, J=52 Hz, 1H), 3.61-3.53 (m, 2H), 2.77 (d, J=17.3 Hz, 1H), 2.49 (m, 1H, merged with DMSO peak), 1.88 (s, 3H), 1.09 (s, 3H), 0.96 (s, 3H).

[α]²⁵=+123.885, c=0.1316, DCM.

Chiral purity (ee %): 100

Peak 2: (5R,7S)-7-fluoro-5,8,8-trimethyl-5-[3-[3-(2,2,2-trifluoroethyl)-4-pyridyl]phenyl]-9,10-dihydro-7H-benzo[b][1,8] naphthyridin-6-one (9B_regio) (15.81 mg, 0.03 mmol, 98.60% purity, 23% yield) as white solid.

LCMS: Rt=1.99 min., (ES⁺) m/z (M+H)⁺=496.2

HPLC: (purity: 98.60%)

¹H NMR (400 MHz, DMSO-d₆) δ 10.04 (s, 1H), 8.62 (s, 1H), 8.57 (s, 1H), 7.99 (s, 1H), 7.47 (d, J=7.2 Hz, 1H), 7.40 (t, J=7.36 Hz, 1H), 7.31 (s, 2H), 7.10 (d, J=7.2 Hz, 1H), 7.03 (d, J=7.16 Hz, 1H), 6.82 (s, 1H), 4.54 (d, J=52 Hz, 1H), 3.61-3.53 (m, 2H), 2.77 (d, J=17.3 Hz, 1H), 2.49 (m, 1H, merged with DMSO peak), 1.88 (s, 3H), 1.09 (s, 3H), 0.96 (s, 3H).

[α]²⁵=−106.404, c=0.1316, DCM.

Chiral purity (ee %): 100

### Step 5b: (5S,7S)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9C_regio) & (5R,7R)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9D_regio)

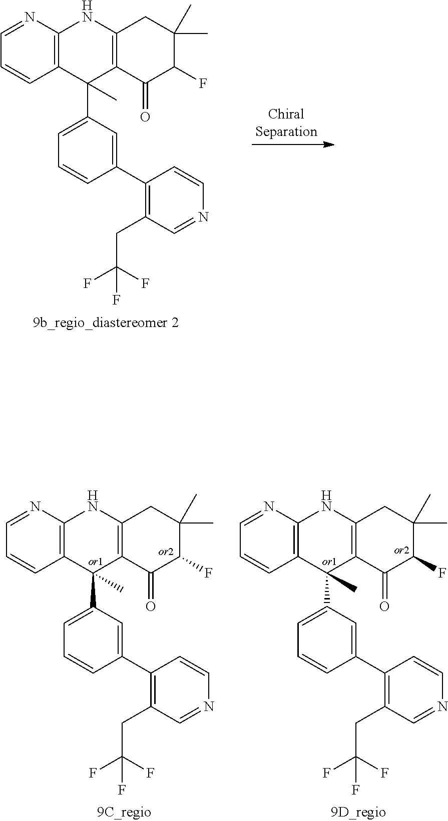

The two enantiomers were separated by normal phase chiral preparatory HPLC Column: CHIRALPAK IG (250×30 mm) 5μ Flow: 27 mL/min Mobile Phase: 50% Hexane+50% of EtOAc, Temp: 35° C.; UV: 335 nm; diluent: DCM to give:

Peak 1:(5S,7S)-7-fluoro-5,8,8-trimethyl-5-[3-[3-(2,2,2-trifluoroethyl)-4-pyridyl]phenyl]-9,10-dihydro-7H-benzo[b][1,8] naphthyridin-6-one (9C_regio) (9.01 mg, 0.02 mmol, 97.93% purity, 16% yield) as white solid.

LCMS: Rt=1.95 min., (ES⁺) m/z (M+H)⁺=496.2

HPLC: (purity: 97.93%)

¹H NMR (400 MHz, DMSO-d₆) δ 10.10 (s, 1H), 8.63 (s, 1H), 8.57 (d, J=4.8 Hz, 1H), 8.00 (dd, J=1.2 Hz, 1H), 7.42-7.38 (m, 2H), 7.30-7.26 (m, 2H), 7.18 (d, J=7.2 Hz, 1H), 7.09 (d, J=6.4 Hz, 1H), 6.85-6.82 (m, 1H), 4.60 (d, J=48.8 Hz, 1H), 3.63-3.56 (m, 2H), 2.70-2.68 (m, 1H), 2.50 (m, 1H, merged with DMSO peak), 1.93 (s, 3H), 1.10 (s, 3H), 0.90 (s, 3H).

[α]²⁵=+57.002, c=0.1316, DCM.

Chiral purity (ee %): 100

Peak 2: (5R,7R)-7-fluoro-5,8,8-trimethyl-5-[3-[3-(2,2,2-trifluoroethyl)-4-pyridyl]phenyl]-9,10-dihydro-7H-benzo[b][1,8]naphthyridin-6-one (9D_regio) (8.61 mg, 0.02 mmol, 96.82% purity, 15% yield) as white solid.

LCMS: Rt=1.95 min., (ES⁺) m/z (M+H)⁺=496.2

HPLC: (purity: 96.82%)

¹H NMR (400 MHz, DMSO-d₆) δ 10.10 (s, 1H), 8.63 (s, 1H), 8.57 (d, J=4.8 Hz, 1H), 8.00 (dd, J=1.2 Hz, 1H), 7.42-7.38 (m, 2H), 7.30-7.26 (m, 2H), 7.18 (d, J=7.2 Hz, 1H), 7.09 (d, J=6.4 Hz, 1H), 6.85-6.82 (m, 1H), 4.60 (d, J=48.8 Hz, 1H), 3.63-3.56 (m, 2H), 2.70-2.68 (m, 1H), 2.50 (m, 1H, merged with DMSO peak), 1.93 (s, 3H), 1.10 (s, 3H), 0.90 (s, 3H). [α]²⁵=−55.482, c=0.1316, DCM.

Chiral purity (ee %): 96.84

### Example 3.21. (S)-5,8,8-trimethyl-5-(3-(methyl(pyrimidin-2-yl)amino)phenyl)-5,8,9,10-tetrahydrobenzo[b][1,8]naphthyridin-6(7H)-one (8A) & (R)-5,8,8-trimethyl-5-(3-(methyl(pyrimidin-2-yl)amino)phenyl)-5,8,9,10-tetrahydrobenzo[b][1,8]naphthyridin-6(7H)-one (8B)

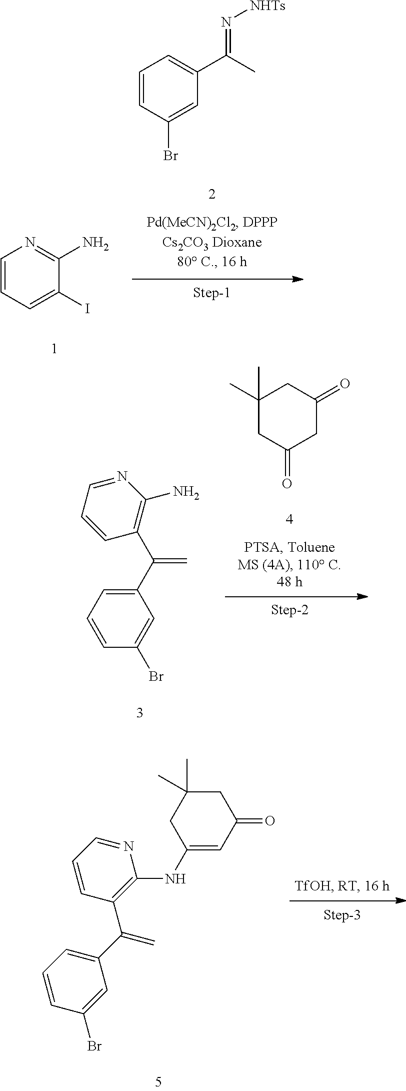

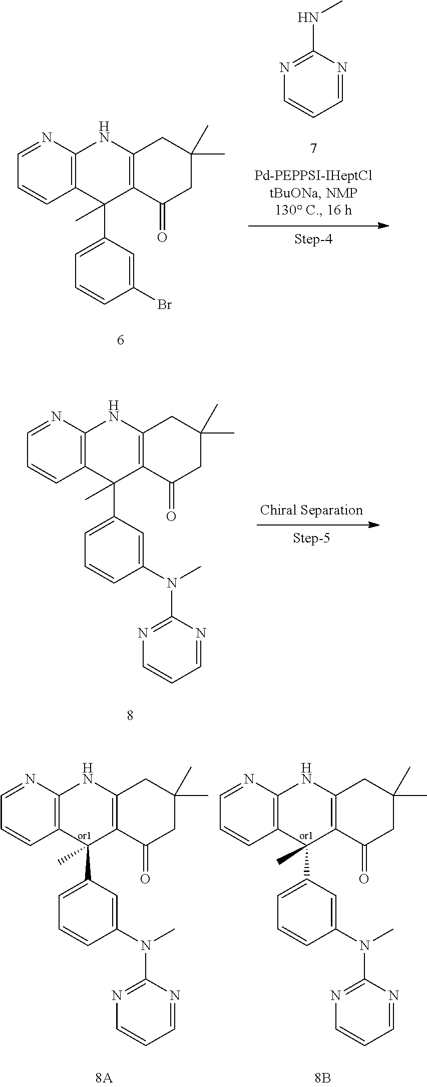

### Step 1: 3-(1-(3-bromophenyl) vinyl) pyridin-2-amine (3)

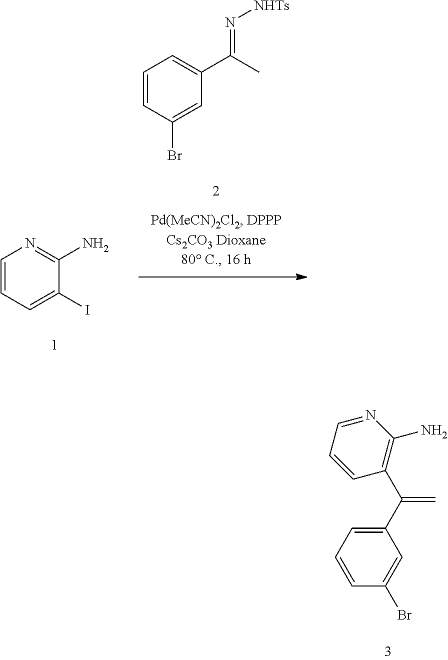

To a degassed stirred solution of 3-iodopyridin-2-amine (1) (1.5 g, 6.82 mmol, 1 eq) and N-[(E)-1-(3-bromophenyl)ethylideneamino]-4-methyl-benzenesulfonamide (2) (5 g, 13.6 mmol, 2 eq) in dioxane (40 mL) were sequentially added Cs₂CO₃ (5.5 g, 17.0 mmol, 2.5 eq), DPPP (562 mg, 1.36 mmol, 0.2 eq) and Pd(MeCN)₂Cl₂ (177 mg, 0.68 mmol, 0.1 eq) at RT under inert atmosphere. The resulting mixture was stirred at 80° C. for 16 h. On completion, the reaction mixture was then diluted with EtOAc and washed sequentially with water and brine. The organic layer was dried over sodium sulfate, filtered and concentrated under reduced pressure to afford a brownish crude. The crude was thus obtained purified by Combi-Flash column chromatography using 30-40% EtOAc in hexane gradient to afford 3-[1-(3-bromo phenyl)vinyl]pyridin-2-amine (3) (600 mg, 3.27 mmol, 88% purity, 28% yield).

LCMS: Rt=3.36 min., (ES⁺) m/z (M+H)=275.2

### Step 2: 3-((3-(1-(3-bromophenyl) vinyl) pyridin-2-yl) amino)-5,5-dimethylcyclohex-2-en-1-one (5)

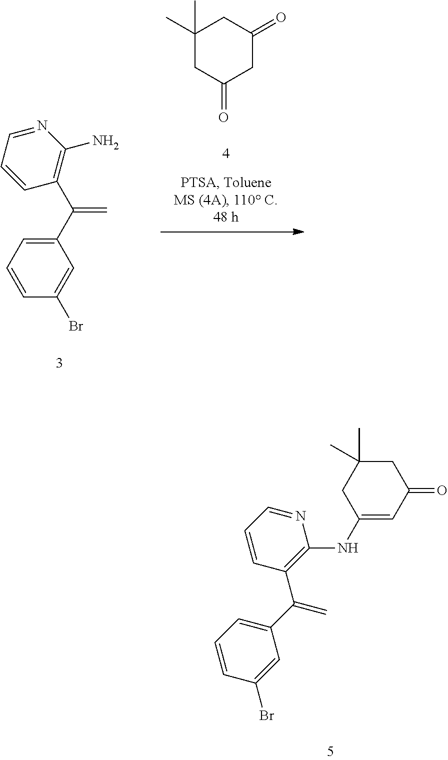

To a stirred solution of 3-[1-(3-bromo phenyl) vinyl]pyridin-2-amine (3) (1.8 g, 6.54 mmol, 1 eq) in toluene (70 ml) were added 5,5-dimethylcyclohexane-1,3-dione (1.4 g, 9.81 mmol, 1.5 eq) and MS 4A (3.5 g) successively. Then the reaction mixture was heated at 110° C. for 48 h. After completion of the reaction, the reaction mixture was evaporated under reduced pressure to afford crude, which was purified by Combi-Flash column chromatography using 40-50% EtOAc in hexane gradient to afford desired product 3-[[3-[1-(3-bromophenyl)vinyl]-2-pyridyl]amino]-5,5-dimethyl-cyclohex-2-en-1-one (5) (900 mg, 2.27 mmol, 72% purity, 25% yield).

LCMS: Rt=3.37 min., (ES⁺) m/z (M+H)⁺=399

### Step 3: 5-(3-bromophenyl)-5,8,8-trimethyl-5,8,9,10-tetrahydrobenzo[b][1,8]naphthyridin-6(7H)-one (6)

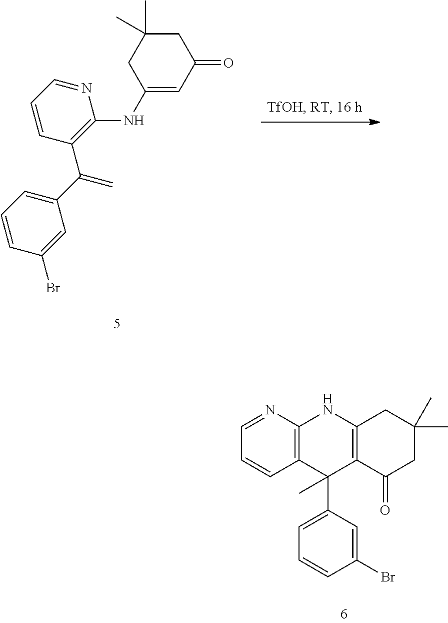 [SMILES: C=C(c1cccc(Br)c1)c1cccnc1NC1=CC(=O)CC(C)(C)C1 ; CC1(C)CC(=O)C2=C(C1)Nc1ncccc1C2(C)c1cccc(Br)c1]

To 3-[[3-[1-(3-bromo phenyl) vinyl]-2-pyridyl]amino]-5,5-dimethyl-cyclohex-2-en-1-one (5)(200 mg, 0.503 mmol, 1 eq) was added TfOH (4 mL) at 0° C. under inert atmosphere. The reaction mixture was then stirred for 16 h at 25° C. After completion of the reaction, the reaction mass was neutralized by sat. NaHCO₃ solution and extracted with Ethyl Acetate. The organic layer was then washed with water, brine and dried with Na2SO4, concentrated under vacuum to afford the crude. The crude thus obtained was purified by Combi-Flash column chromatography using 40-50% EtOAc in hexane gradient to afford desired product (5-(3-bromophenyl)-5,8,8-trimethyl-9,10-dihydro-7H-benzo[b][1,8]naphthyridin-6-one (6) (140 mg, 0.352 mmol, 90% purity, 63% yield).

LCMS: Rt=1.84 min., (ES⁺) m/z (M+H)=399.2

### Step 4: 5,8,8-trimethyl-5-(3-(methyl(pyrimidin-2-yl) amino) phenyl)-5,8,9,10-tetrahydrobenzo[b][1,8]naphthyridin-6(7H)-one (8)

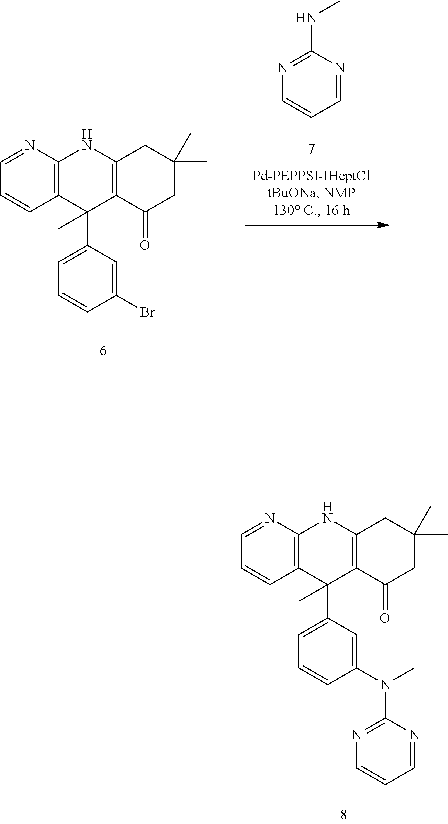

To a stirred solution of 5-(3-bromophenyl)-5,8,8-trimethyl-9,10-dihydro-7H-benzo[b][1,8] naphthyridin-6-one (6) (250 mg, 0.629 mmol, 1 eq) in NMP (5 mL) was added N-methylpyrimidin-2-amine (7) (275 mg, 2.52 mmol, 4 eq) and sodium tert-butoxide (612 mg, 1.89 mmol, 3 eq) and degassed for 15 mins. After that, Pd-PEPPSI-IHeptCl (60 mg, 0.126 mmol, 0.2 eq) was added to the reaction mixture and stirred for 16 h at 130° C. in seal tube. On completion, chilled water was added to the reaction mixture and extracted with EtOAc. The organic layer was then washed with water, brine and dried with Na2SO4, concentrated under vacuum to afford the crude. The crude thus obtained was purified by reverse phase column chromatography to afford 5,8,8-trimethyl-5-(3-(methyl(pyrimidin-2-yl) amino) phenyl)-5,8,9,10-tetrahydrobenzo[b][1,8]naphthyridin-6(7H)-one (8) (60 mg, 0.137 mmol, 97% purity, 22% yield)

LCMS: Rt=2.03 min., (ES⁺) m/z (M+H)=426.2.

### Step 5: (S)-5,8,8-trimethyl-5-(3-(methyl(pyrimidin-2-yl)amino)phenyl)-5,8,9,10-tetrahydrobenzo[b][1,8]naphthyridin-6(7H)-one (8A) (R)-5,8,8-trimethyl-5-(3-(methyl(pyrimidin-2-yl) amino) phenyl)-5,8,9,10-tetrahydrobenzo[b] [1,8]naphthyridin-6(7H)-one (8B)

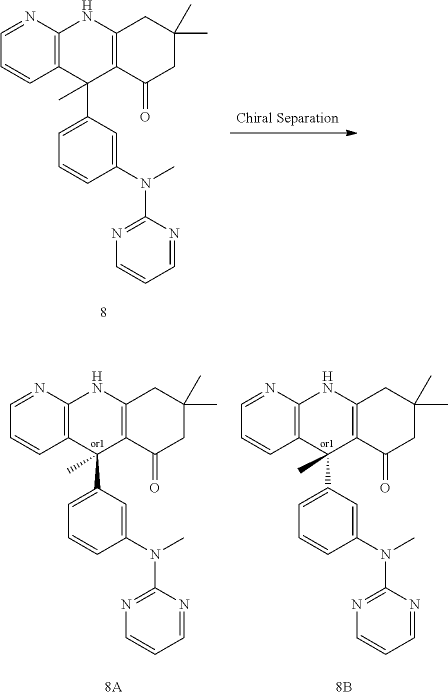

Chiral separation of rac-5,8,8-trimethyl-5-(3-(methyl(pyrimidin-2-yl) amino) phenyl)-5,8,9,10-tetrahydrobenzo[b][1,8]naphthyridin-6(7H)-one (8) was done in waters SFC prep 80 instrument equipped with waters 2489 uv/visible detector by using chiralcel ox-h (21.1 mm×250 mm), 5μ column operating at 35° C. temperature, maintaining flow rate of 60 ml/min, using 70% CO2 in super critical state and 30% of (100% methanol) as mobile phase, run this isocratic mixture up to 8.0 minutes and also maintained the isobaric condition of 100 bar at 240 nm.

SFC fractions were collected and evaporated under reduced pressure to afford:

Peak 1: (S)-5,8,8-trimethyl-5-(3-(methyl(pyrimidin-2-yl)amino)phenyl)-5,8,9,10-tetrahydrobenzo[b][1,8]naphthyridin-6(7H)-one (8A) (4.92 mg, 98.93% purity, 8.11% yield)

UPLC: Rt=1.83 min, (ES+) m/z (M+H)+=426.2.

1H NMR (400 MHz, DMSO-d6) δ 9.76 (s, 1H), 8.32 (d, 2H), 7.96-7.94 (m, 1H), 7.26 (s, 1H), 7.21-7.13 (m, 3H), 7.04 (d, 1H), 6.84-6.80 (m, 1H), 6.70 (t, 1H), 3.40 (s, 3H), 2.47 (s, 2H), 2.07-1.95 (m, 2H), 1.89 (s, 3H), 1.01 (d, 6H)

[α]²⁵=−28.748, c=0.5079, CH3CN.

Chiral purity (ee %): 100

Peak 2: (R)-5,8,8-trimethyl-5-(3-(methyl(pyrimidin-2-yl) amino) phenyl)-5,8,9,10-tetrahydrobenzo[b][1,8] naphthyridin-6(7H)-one (8B) (5.94 mg, 96.72% purity, 9.57% yield).

UPLC: Rt=1.84 min, (ES+) m/z (M+H)+=426.2.

1H NMR (400 MHz, DMSO-d6) δ 9.76 (s, 1H), 8.32 (d, 2H), 7.96-7.94 (m, 1H), 7.26 (s, 1H), 7.21-7.13 (m, 3H), 7.04 (d, 1H), 6.84-6.80 (m, 1H), 6.70 (t, 1H), 3.40 (s, 3H), 2.47 (s, 2H), 2.07-1.95 (m, 2H), 1.89 (s, 3H), 1.01 (d, 6H)

[α]²⁵=20.085, c=0.5128, CH3CN.

Chiral purity (ee %): 100

### Example 3.22. (S)-5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10A) and (R)-5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10B)

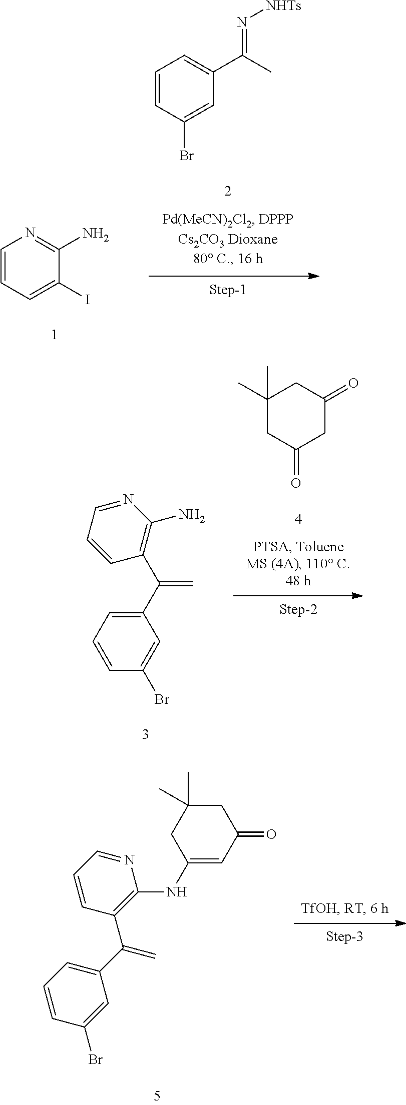

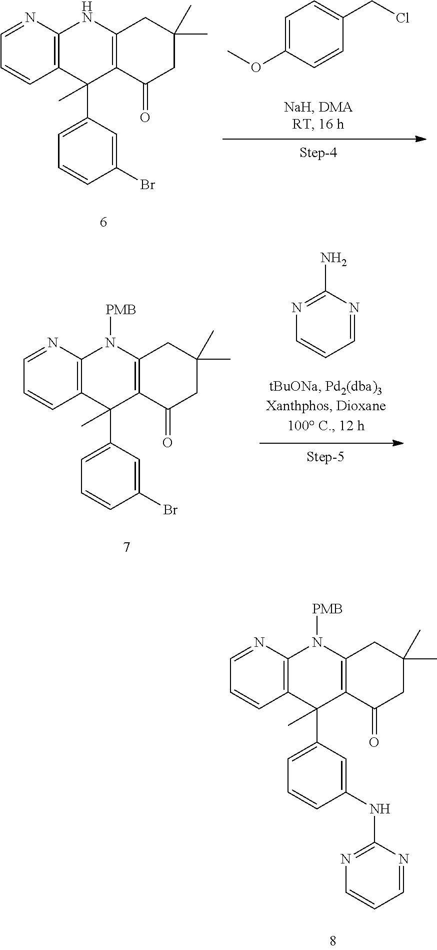

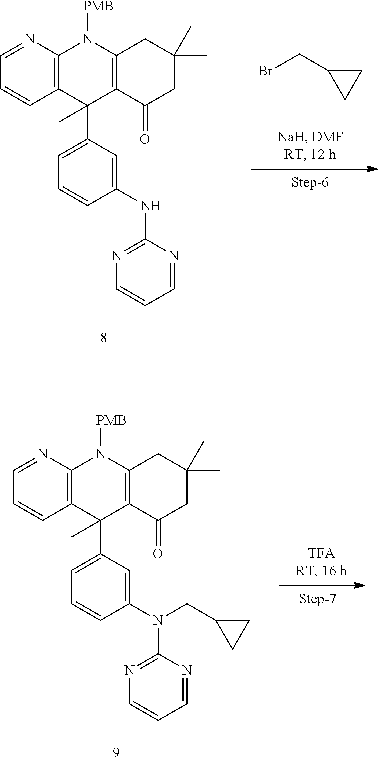

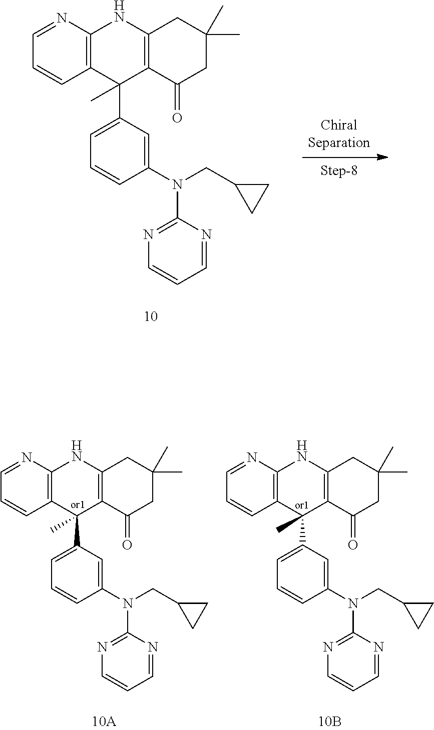 [SMILES: CC1(C)CC(=O)C2=C(C1)Nc1ncccc1C2(C)c1cccc(N(CC2CC2)c2ncccn2)c1 ; CC1(C)CC(=O)C2=C(C1)Nc1ncccc1[C@]2(C)c1cccc(N(CC2CC2)c2ncccn2)c1 ; CC1(C)CC(=O)C2=C(C1)Nc1ncccc1[C@@]2(C)c1cccc(N(CC2CC2)c2ncccn2)c1]

### Step 1: 3-(l-(3-bromophenyl) vinyl) pyridin-2-amine (3)

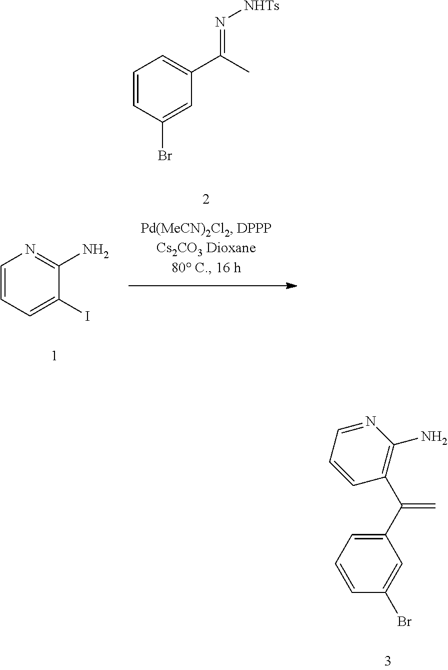

To a degassed stirred solution of 3-iodopyridin-2-amine (1) (1.5 g, 6.82 mmol, 1 eq) and N-[(E)-1-(3-bromophenyl)ethylideneamino]-4-methyl-benzenesulfonamide (2) (5 g, 13.6 mmol, 2 eq) in dioxane (40 mL) were sequentially added Cs₂CO₃ (5.5 g, 17.0 mmol, 2.5 eq), DPPP (562 mg, 1.36 mmol, 0.2 eq) and Pd(MeCN)₂Cl₂ (177 mg, 0.68 mmol, 0.1 eq) at RT under inert atmosphere. The resulting mixture was then stirred at 80° C. for 16 h. On completion, the reaction mixture was then diluted with EtOAc and washed sequentially with water and brine. The organic layer was dried over Na2SO4, filtered and concentrated under reduced pressure to afford a brownish crude. The crude was thus obtained purified by Combi-Flash column chromatography using 30-40% EtOAc in hexane gradient to afford 3-[1-(3-bromo phenyl)vinyl]pyridin-2-amine (3) (600 mg, 3.27 mmol, 88% purity, 28% yield).

LCMS: Rt=3.36 min., (ES⁺) m/z (M+H)=275.2

### Step 2: 3-((3-(1-(3-bromophenyl) vinyl) pyridin-2-yl) amino)-5,5-dimethylcyclohex-2-en-1-one (5)

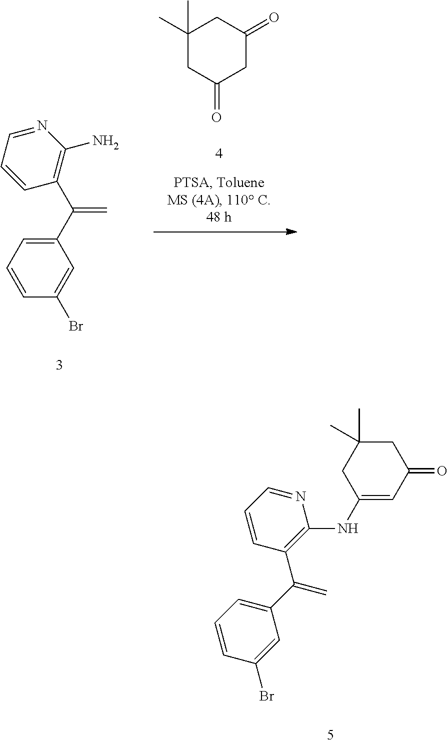

To a stirred solution of 3-[1-(3-bromo phenyl) vinyl]pyridin-2-amine (3) (1.8 g, 6.54 mmol, 1 eq) in toluene (70 ml) were added 5,5-dimethylcyclohexane-1,3-dione (4) (1.4 g, 9.81 mmol, 1.5 eq) and MS 4A (3.5 g) successively. Then the reaction mixture was heated at 110° C. for 48 h. After completion of the reaction, the reaction mixture was evaporated under reduced pressure to afford crude, which was purified by Combi-Flash column chromatography using 40-50% EtOAc in hexane gradient to afford desired product 3-[[3-[1-(3-bromophenyl)vinyl]-2-pyridyl]amino]-5,5-dimethyl-cyclohex-2-en-1-one (5) (900 mg, 2.27 mmol, 72% purity, 25% yield).

LCMS: Rt=3.37 min., (ES⁺) m/z (M+H)⁺=399

### Step 3: 5-(3-bromophenyl)-5,8,8-trimethyl-5,8,9,10-tetrahydrobenzo[b][1,8]naphthyridin-6(7H)-one (6)

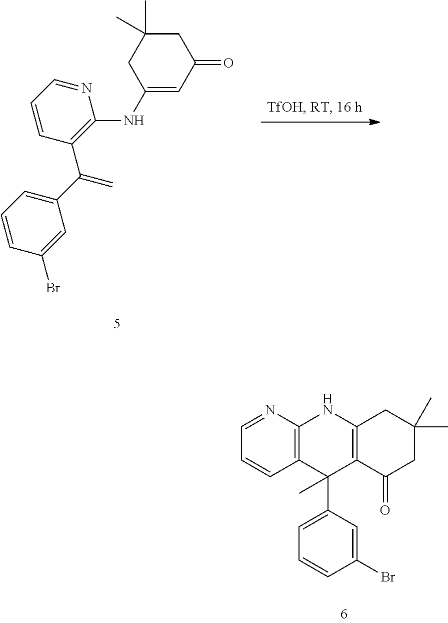 [SMILES: C=C(c1cccc(Br)c1)c1cccnc1NC1=CC(=O)CC(C)(C)C1 ; CC1(C)CC(=O)C2=C(C1)Nc1ncccc1C2(C)c1cccc(Br)c1]

To 3-[[3-[l-(3-bromo phenyl) vinyl]-2-pyridyl]amino]-5,5-dimethyl-cyclohex-2-en-1-one (5) (200 mg, 0.503 mmol, 1 eq) was added TfOH (4 mL) at 0° C. under inert atmosphere. The reaction mixture was then stirred for 16 h at 25° C. After completion of the reaction, the reaction mass was neutralized by sat. NaHCO3 solution and extracted with Ethyl Acetate. The organic layer was then washed with water, brine and dried with Na2SO4, concentrated under vacuum to afford the crude. The crude thus obtained was purified by Combi-Flash column chromatography using 40-50% EtOAc in hexane gradient to afford desired product (5-(3-bromophenyl)-5,8,8-trimethyl-9,10-dihydro-7H-benzo[b][1,8]naphthyridin-6-one (6) (140 mg, 0.352 mmol, 90% purity, 63% yield).

LCMS: Rt=1.84 min., (ES⁺) m/z (M+H)=399.2

### Step 4: 5-(3-bromophenyl)-10-(4-methoxybenzyl)-5,8,8-trimethyl-5,8,9,10-tetrahydrobenzo[b][1,8]naphthyridin-6(7H)-one (7)

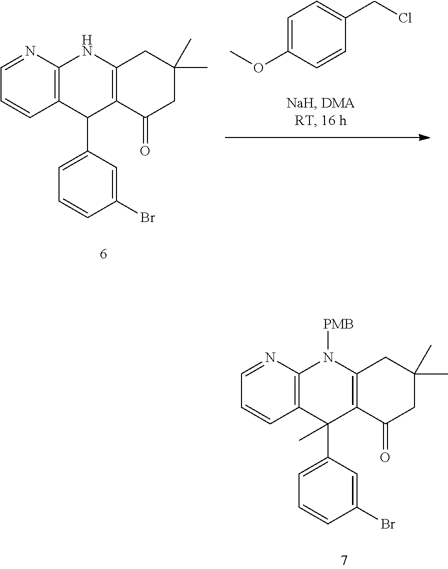

To a stirred solution of 5-(3-bromophenyl)-5,8,8-trimethyl-9,10-dihydro-7H-benzo[b][1,8]naphthyridin-6-one (6) (300 mg, 0.755 mmol, 1 eq) in DMA (5 mL) was added NaH (60% in oil) (24 mg, 0.982 mmol, 1.3 eq) at 0° C. After 15 mins PMB-Cl (0.15 mL, 1.13 mmol, 1.5 eq) was added to the reaction mixture and stirred for 16 h at 25° C. On completion, chilled water was added to reaction mixture and extracted with EtOAc. The organic layer was then washed with water, brine and dried with Na2SO4, concentrated under vacuum to afford the crude. The crude thus obtained was purified by Combi-Flash column chromatography using 20-25% EtOAc in hexane gradient to afford desired product 5-(3-bromophenyl)-10-[(4-methoxyphenyl)methyl]-5,8,8-trimethyl-7,9-dihydrobenzo[b][1,8]naphthyridin-6-one (7) (320 mg, 0.618 mmol, 95% purity, 77% yield).

LCMS: Rt=2.25 min., (ES⁺) m/z (M+H)=519

### Step 5: 10-(4-methoxybenzyl)-5,8,8-trimethyl-5-(3-(pyrimidin-2-ylamino) phenyl)-5,8,9,10-tetrahydrobenzo[b][1,8]naphthyridin-6(7H)-one (8)

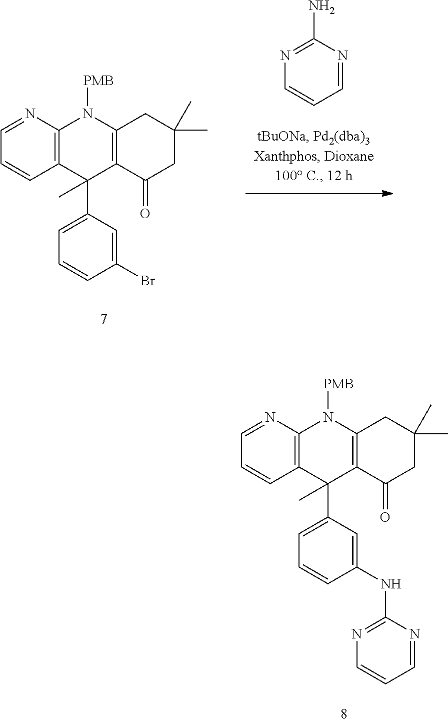

To a stirred solution of 5-(3-bromophenyl)-10-[(4-methoxyphenyl) methyl]-5,8,8-trimethyl-7,9-dihydrobenzo[b][1,8]naphthyridin-6-one (7) (850 mg, 1.64 mmol, 1.00 eq), pyrimidin-2-amine (391 mg, 4.11 mmol, 2.50 eq) in 1,4 dioxane (10 ml) was added tBuONa (1597 mg, 4.93 mmol, 3.00 eq) and argon was purged for 10 min. After that Pd2(dba)3 (226 mg, 0.246 mmol, 0.15 eq) and Xantphos (285 mg, 0.493 mmol, 0.3 eq) were added and the reaction was stirred at 100° C. for 12 h. After completion of the reaction, which was monitored by TLC and LCMS, the reaction mixture was quenched with ice-water and extracted with EtOAc. The organic layer was then washed with water, brine and dried with Na2SO4, concentrated under vacuum to afford the crude. The crude thus obtained was purified by Combi-Flash column chromatography using 30-40% EtOAc in hexane gradient to afford desired product 10-[(4-methoxyphenyl)methyl]-5,8,8-trimethyl-5-[3-(pyrimidin-2-ylamino)phenyl]-7,9-dihydrobenzo[b][1,8]naphthyridin-6-one (8) (520 mg, 0.978 mmol, 95% purity, 56% yield).

LCMS: Rt=3.66 min., (ES⁺) m/z (M+H)=532.2

### Step 6: 5-(3-((cyclopropylmethyl)(pyrimidin-2-yl)amino)phenyl)-10-(4-methoxybenzyl)-5,8,8-trimethyl-5,8,9,10-tetrahydrobenzo[b][1,8]naphthyridin-6(7H)-one (9)

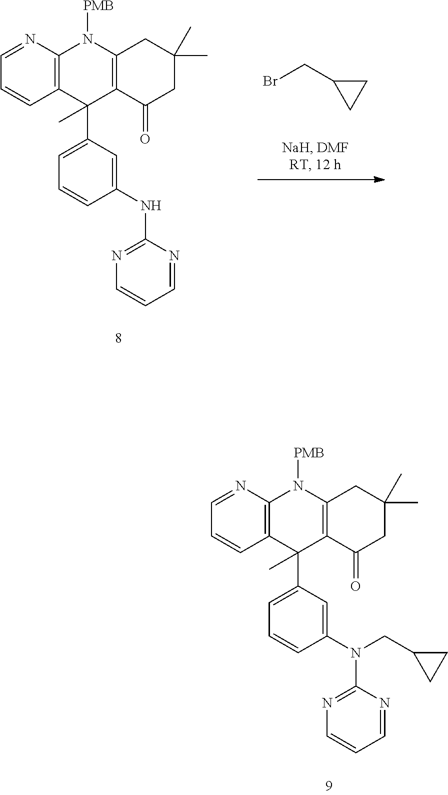

To a stirred solution of 10-[(4-methoxyphenyl)methyl]-5,8,8-trimethyl-5-[3-(pyrimidin-2-ylamino)phenyl]-7,9-dihydrobenzo[b][1,8]naphthyridin-6-one (8) (250 mg, 0.470 mmol, 1 eq) in DMF (5 mL) was added sodium Hydride (60% in oil) (14 mg, 0.564 mmol, 1.2 eq) at 0° C. and stirred for 20 min. After that bromomethyl cyclopropane (63 mg, 0.47 mmol, 1 eq) was added and the reaction was stirred at RT for 14 h. After completion of the reaction, which was monitored by TLC and LCMS, the reaction mixture was quenched with ice-water and extracted with EtOAc. The organic layer was then washed with water, brine and dried with Na2SO4, concentrated under vacuum to afford the crude. The crude thus obtained was purified by Combi-Flash column chromatography using 1-2% MeOH in DCM gradient to afford 5-[3-[cyclopropylmethyl(pyrimidin-2-yl)amino]phenyl]-10-[(4-methoxyphenyl)methyl]-5,8,8-trimethyl-7,9-dihydrobenzo[b][1,8]naphthyridin-6-one (9) (190 mg, 0.324 mmol, 86% purity, 60% yield).

LCMS: Rt=2.19 min., (ES⁺) m/z (M+H)=586.1

### Step 7: 5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10)

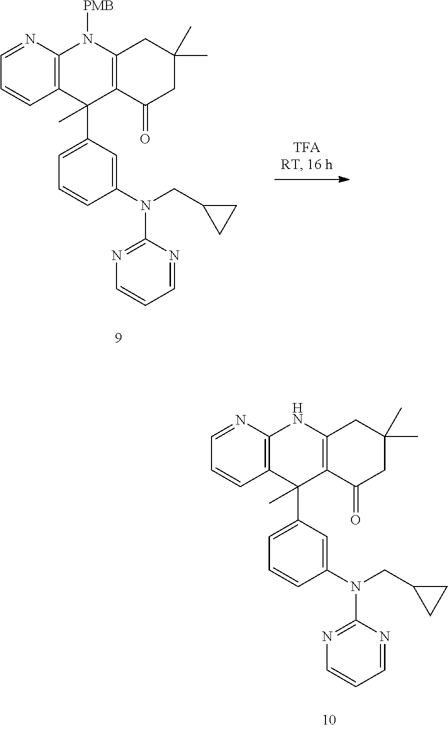

To 5-[3-[cyclopropylmethyl(pyrimidin-2-yl)amino]phenyl]-10-[(4-methoxyphenyl)methyl]-5,8,8-trimethyl-7,9-dihydrobenzo[b][1,8]naphthyridin-6-one (9) (190 mg, 0.324 mmol, 1 eq) was added TFA (4 mL) at 0° C. under inert atmosphere. The reaction mixture was then stirred for 16 h at 25° C. After completion of the reaction, the reaction mass was neutralized by sat. NaHCO3 solution and extracted with 10% MeOH in DCM. The organic layer was then washed with water, brine and dried with Na2SO4, concentrated under vacuum to afford the crude. The crude thus obtained was purified by Combi-Flash column chromatography using 2-3% MeOH in DCM to afford 5-[cyclopropylmethyl(pyrimidin-2-yl)amino]phenyl]-5,8,8-trimethyl-9,10-dihydro-7H-benzo[b][1,8]naphthyridin-6-one (10) (90 mg, 0.193 mmol, 100% purity, 60% yield).

LCMS: Rt=2.17 min., (ES⁺) m/z (M+H)=466

### Step 8: (S)-5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10A) and (R)-5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10B)

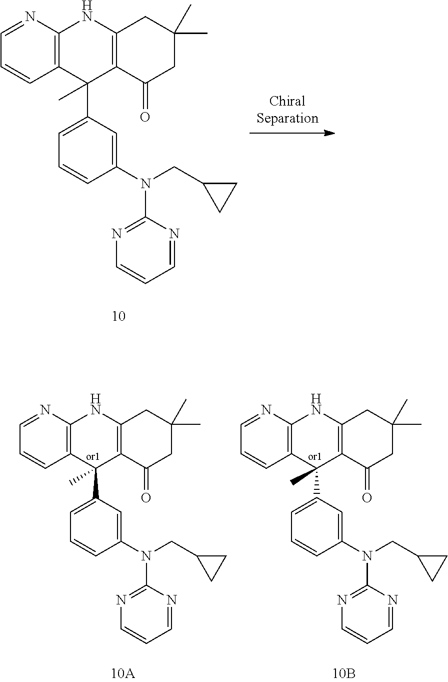

Chiral separation of rac-(S)-5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10) was done on Agilent 1200 series instrument. Column name: CHIRALPAK IG (250×21 mm) 5u. Operating at ambient temperature and flow rate is 21.0 mL/min. Mobile phase was mixture of 60% Hexane, 20% Ethyl acetate and 20% Ethyl alcohol, held this isocratic mixture run up-to 20 min with wavelength of 336 nm.

SFC fractions were collected and evaporated under reduced pressure to afford:

Peak 1: (S)-5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10A) (21.58 mg, 98.61% purity, 25% yield)

UPLC: Rt=2.12 min, (ES+) m/z (M+H)+=466.3.

¹H NMR (400 MHz, MeOD) δ 8.23 (d, 2H), 7.98-7.94 (m, 1H), 7.38-7.27 (m, 3H), 7.27-7.21 (m, 11H), 7.02 (d, 1H), 6.87-6.78 (m, 1H), 6.61 (t, 1H), 3.97-3.88 (m, 1H), 3.71-3.62 (m, 1H), 2.53 (s, 2H), 2.18-2.05 (m, 2H), 1.96 (s, 3H), 1.11 (s, 4H), 1.07 (s, 3H), 0.39-0.29 (m, 2H), 0.12-0.01 (m, 2H).

[α]²⁵=27.814, c=0.2518, CH3CN.

Chiral purity (ee %): 100

Peak 2: (R)-5-(3-((cyclopropylmethyl)(pyrimidin-2-yl)amino)phenyl)-5,8,8-trimethyl-5,8,9,10-tetrahydrobenzo[b][1,8]naphthyridin-6(7H)-one (10B) (20 mg, 99.83% purity, 23% yield)

UPLC: Rt=2.14 min, (ES+) m/z (M+H)+=466.3.

¹H NMR (400 MHz, MeOD) δ 8.23 (d, 2H), 7.98-7.94 (m, 1H), 7.38-7.27 (m, 3H), 7.27-7.21 (m, 1H), 7.02 (d, 1H), 6.87-6.78 (m, 1H), 6.61 (t, 1H), 3.97-3.88 (m, 1H), 3.71-3.62 (m, 1H), 2.53 (s, 2H), 2.18-2.05 (m, 2H), 1.96 (s, 3H), 1.11 (s, 4H), 1.07 (s, 3H), 0.39-0.29 (m, 2H), 0.12-0.01 (m, 2H).

[α]²⁵=−29.654, c=0.2529, CH3CN.

Chiral purity (ee %): 100

### Example 4. GSK3 Inhibition of Select Compounds

TR-FRET assay was performed in a white, 384 well, low volume, flat bottom plate (Grenier). After a 30 minute preincubation in assay buffer with the indicated concentration of compound, the kinase activity was initiated by the addition of the substrates. 1 nM GSK3a (GST-full length GSK3a [SignalChem G08-10G]) or 1 nM GSK3b (GST-full length GSK3b: [SignalChem G08-9G]) were then incubated at ambient temperature for 100 minutes in 10 μl of 50 mM Tris, pH 7.5, 20 mM MgCl₂, 0.05 mM DTT, 100 μg/ml BSA and 1% DMSO, with 200 nM biotinylated peptide substrate (biotinylated and S645 phosphorylated glycogen synthase 631-650) and ATP (e.g., at 2 μM, 4 μM, 10 μM, 100 μM, or 1 mM). The kinase activity was quenched with 2 μl of 250 mM EDTA. A 10 μl volume of 40 nM Strepavidin-d2, 2 nM Tb²⁺-pSer641 antibody in TR-FRET Detection Buffer (Invitrogen) was then added. After 60 min at room temperature the plate was read on Envision plate reader using Ex: 340 nm, Em: 615 nM and nM. The normalized 665/615 Signal ratio versus log compound concentration was analyzed by XLFIT to yield IC₅₀ values. Exemplary results are shown in Tables 3 and 4.

### Example 5. Human Microsomal of Stability of Select Compounds

Stability of compound (5 μL of a 10 mM DMSO solution) in human liver microsomes was tested in duplicate by incubating compound at 1 μM for 60 minutes at 37° C. Compound level at 60 minutes was compared to level at 0 minutes, and percent remaining was calculated. Order by selecting Microsomal Stability in Prometheus, checking boxes for desired species. Assay Controls: microsomes with NADPH without compound-matrix interference. Microsomes plus compound without NADPH—non-enzymatic instability. Exemplary results are shown in Tables 3 and 4.

### Example 6. Permeability of Select Compounds

The MDR1/MDCK assay is run at WuXi and Absorption Systems. This assay is used to determine the blood-brain barrier (BBB) penetration potential of a test compound using MDR1-MDCK cell monolayers. Catalog number EA203. Exemplary results are shown in Tables 3 and 4.

### Deliverables

The percent recovery of the test compound from the Transwell® wells containing MDR1-MDCK cell monolayers
The apparent permeability (Pₐₚₚ) in both directions
The efflux ratio (Pₐₚₚ B to A)/(Pₐₚₚ A to B)
The blood-brain barrier penetration potential classification:
   High when
      Pₐₚₚ A to B≥3.0×10⁻⁶ cm/s, and efflux<3.0
   Moderate when
      Pₐₚₚ A to B≥3.0×10⁻⁶ cm/s, and 10>efflux≥3.0
   Low when either
      Pₐₚₚ A to B≥3.0×10⁻⁶ cm/s, and efflux 210, or when
      Pₐₚₚ A to B<3.0×10⁴ cm/s.

### Substrate

Test compound at 5 μM in HBSSg with maximum DMSO concentration not greater than 1%.

### Assay System

Confluent monolayers of MDR1-MDCK cells, 7 to 11 days old.

### Assay Conditions

Receiver well with 1% BSA in modified Hanks buffer (HBSSg)
Apical and basolateral side at pH 7.4
Dose two monolayers in each direction (N=2)
Dose apical side for (A to B) assessment
Dose basolateral side for (B to A) assessment
Sample both apical and basolateral sides at 120 minutes
Determine the concentrations of test compound using a generic LC-MS/MS method with a minimum 4 point calibration curve.

### Assay QC

The quality of the cell monolayer batch is verified using control compounds before monolayers are released for use
The quality of each monolayer used in the assay is verified by a TEER measurement and by calculating the Pₐₚₚ for a control compound.

In Tables 3 and 4, A: ≤0.10 μM; B: >0.10 to ≤1.0 μM; C: >1.0 to ≤5.0 μM; D: >5.0 to ≤20 μM; E: >20 to ≤60 μM; and F: >5.0 μM.

TABLE 3
Exemplary data of select compounds

| | GSK3a TR- | GSK3b TR- | GSK3b 10 | GSK3b 100 | GSK3a 10 |
|---|---|---|---|---|---|
| | FRET 1 mM | FRET 1 mM | μM ATP | μM ATP | μM ATP |
| Compound | ATP Average | ATP Average | Average | Average | Average |
| No. | IC₅₀ (μM) | IC₅₀ (μM) | IC₅₀ (μM) | IC₅₀ (μM) | IC₅₀ (μM) |
| 101A | C | C | A | B | A |
| 101B | | | C | E | D |
| 102A | C | C | A | B | A |
| 103A | D | C | | B | |
| 104A | C | C | | B | |
| 105A | | D | | | |
| 105B | C | C | | | |
| 107A | | D | | | |
| 107B | C | C | | | |
| 108A | B | B | | | |
| 108B | D | | | | |
| 109A | C | C | | | |
| 109B | D | D | | | |
| 110A | C | D | | | |
| 111A | B | B | | A | |
| 112A | C | C | | A | |
| 113A | | C | | B | |
| 114A | B | C | A | A | A |
| 115A | D | D | | | |
| 116A | D | | | | |
| 116B | B | B | | | |
| 117A | D | C | | B | |
| 118A | | D | | | |
| 118B | C | C | | | |
| 119A | D | C | | B | |
| 120A | C | B | | | |
| 121A | A | A | | | |
| 121B | C | C | | | |
| 122A | | D | | C | |
| 123A | C | C | | | |
| 123B | C | C | | | |
| 124A | D | D | | D | |
| 124B | D | D | | D | |
| 125A | B | B | | | |
| 125B | C | C | | | |
| 126A | | | | | |
| 126B | | | | | |
| 127A | B | B | | | |
| 127B | D | D | | | |
| 128A | D | C | | | |
| 128B | C | C | | | |
| 129A | | D | D | E | E |
| 129B | D | D | D | E | E |
| 130A | D | D | A | B | A |
| 130B | D | D | B | B | B |
| 131A | | | | | |
| 131B | C | C | | | |
| 132A | C | C | | | |
| 132B | D | C | | | |
| 133A | C | C | A | B | A |
| 133B | | | | | |
| 134A | | | | | |
| 135A | B | B | | | |
| 135B | D | C | | | |
| 136A | D | D | | | |
| 136B | D | D | | | |
| 137A | C | B | | | |
| 137B | C | C | | | |
| 138A | | | | | |
| 138B | | | | | |
| 139A | A | A | | | |
| 139B | D | D | | | |
| 140A | C | B | | | |
| 140B | D | D | | | |
| 141A | D | C | | | |
| 141B | C | C | | | |
| 142A | | | | | |
| 142B | | | | | |
| 143A | | | | | |
| 143B | | | | | |
| 144A | B | B | | | |
| 144B | D | D | | | |
| 145A | C | C | | | |
| 145B | D | D | | | |
| 146A | | | | | |
| 146B | | | | | |
| 147A | B | C | | | |
| 148A | A | A | | | |
| 148B | D | D | | | |
| 149A | B | B | A | A | A |
| 150A | B | B | A | A | A |
| 151A | C | C | | | |
| 152A | C | D | | | |
| 153A | A | A | | | |
| 153B | D | D | | | |
| 154A | | | | | |
| 154B | D | D | | | |
| 155A | B | C | | | |
| 155B | D | D | | | |
| 156A | A | B | | | |
| 156B | D | D | | | |
| 157A | C | B | | | |
| 157B | D | D | | | |
| 158A | B | B | | | |
| 159A | | | | | |
| 159B | | | | | |
| 160A | B | B | | | |
| 161A | | | | | |
| 161B | B | C | | | |
| 162A | D | C | | | |
| 162B | B | B | | | |
| 163A | B | B | | | |
| 164A | C | C | | | |
| 165A | B | B | A | A | A |
| 166A | B | B | | | |
| 166B | D | D | | | |
| 167A | B | B | | | |
| 167B | C | C | | | |
| 168A | B | B | | | |
| 169A | B | B | | | |
| 170A | C | C | | | |
| 170B | D | C | | | |
| 171A | C | C | | | |
| 171B | C | B | | | |
| 172A | B | B | | | |
| 173A | C | C | | | |
| 174A | C | C | | | |
| 174B | C | B | | | |
| 175A | | | | | |
| 176A | D | D | | | |
| 176B | D | D | | | |

TABLE 3
Biochemical data of select compounds

| | | | | | MIDCK |
|---|---|---|---|---|---|
| | | | MDCK | MDCK | Permeability |
| | | Microsome | Permeability | Permeability | (MDR1- |
| | | Stability | (MDR1- | (MDR1- | MDCK); |
| | GSK3a 100 | (Human) | MDCK); | MDCK); | Mean; P. |
| | μM ATP | (DMPK_BIIB); | Mean; | Mean; | Ratio(B-A/ |
| Compound | Average | Mean; in vitro | Papp(A-B) | Papp(B-A) | A-B) Efflux |
| No. | IC₅₀ (μM) | t_{1/2} (min) | (10⁻⁶ cm/s) | (10⁻⁶ cm/s) | Ratio |
| 101A | B | 37 | 30 | 22 | 0.72 |
| 101B | E | | | | |
| 102A | B | 14 | 22 | 19 | 0.87 |
| 103A | B | 111 | 30 | 16 | 0.56 |
| 104A | A | | | | |
| 105A | | | | | |
| 105B | | | | | |
| 107A | | | | | |
| 107B | | 94 | 30 | 18 | 0.61 |
| 108A | | | 18 | 56 | 3.2 |
| 108B | | | | | |
| 109A | | | | | |
| 109B | | | | | |
| 110A | | | | | |
| 111A | A | | | | |
| 112A | A | | | | |
| 113A | C | | | | |
| 114A | A | 49 | 23 | 16 | 0.68 |
| 115A | | 7.0 | 29 | 22 | 0.76 |
| 116A | | | | | |
| 116B | | | | | |
| 117A | B | | | | |
| 118A | | | | | |
| 118B | | | | | |
| 119A | B | | | | |
| 120A | | | | | |
| 121A | | | 22 | 63 | 2.9 |
| 121B | | | | | |
| 122A | C | | | | |
| 123A | | | | | |
| 123B | | | | | |
| 124A | D | | | | |
| 124B | D | | | | |
| 125A | | | | | |
| 125B | | | | | |
| 126A | | | | | |
| 126B | | | | | |
| 127A | | 8.3 | 17 | 8.7 | 0.50 |
| 127B | | | | | |
| 128A | | | | | |
| 128B | | >145 | 20 | 10 | 0.53 |
| 129A | | 11 | 2.1 | 1.2 | 0.60 |
| 129B | | 1.1 | 1.8 | 1.0 | 0.59 |
| 130A | B | 0.55 | 1.4 | 0.65 | 0.48 |
| 130B | C | 0.53 | 1.3 | 0.75 | 0.60 |
| 131A | | | | | |
| 131B | | | | | |
| 132A | | | | | |
| 132B | | | | | |
| 133A | B | 78 | 14 | 8.2 | 0.56 |
| 133B | | | | | |
| 134A | | | | | |
| 135A | | | | | |
| 135B | | | | | |
| 136A | | | | | |
| 136B | | | | | |
| 137A | | | | | |
| 137B | | | | | |
| 138A | | | | | |
| 138B | | | | | |
| 139A | | | 0.52 | 75 | 144 |
| 139B | | | | | |
| 140A | | | | | |
| 140B | | | | | |
| 141A | | | | | |
| 141B | | | | | |
| 142A | | | | | |
| 142B | | | | | |
| 143A | | | | | |
| 143B | | | | | |
| 144A | | | | | |
| 144B | | | | | |
| 145A | | | | | |
| 145B | | | | | |
| 146A | | | | | |
| 146B | | | | | |
| 147A | | | | | |
| 148A | | | | | |
| 148B | | | | | |
| 149A | A | 16 | 10 | 15 | 3.5 |
| 150A | A | 1.9 | 20 | 17 | 0.93 |
| 151A | | | | | |
| 152A | | | | | |
| 153A | | | | | |
| 153B | | | | | |
| 154A | | 6.4 | 15 | 9.5 | 0.62 |
| 154B | | | | | |
| 155A | | | | | |
| 155B | | | | | |
| 156A | | | | | |
| 156B | | | | | |
| 157A | | | | | |
| 157B | | | | | |
| 158A | | | | | |
| 159A | | | | | |
| 159B | | | | | |
| 160A | | | | | |
| 161A | | 10 | 12 | 6.9 | 0.59 |
| 161B | | | | | |
| 162A | | | | | |
| 162B | | | | | |
| 163A | | | | | |
| 164A | | | | | |
| 165A | A | | | | |
| 166A | | | | | |
| 166B | | | | | |
| 167A | | | | | |
| 167B | | | | | |
| 168A | | 33 | 2.5 | 2.2 | 0.90 |
| 169A | | | | | |
| 170A | | | | | |
| 170B | | | | | |
| 171A | | | | | |
| 171B | | | | | |
| 172A | | | | | |
| 173A | | | | | |
| 174A | | | | | |
| 174B | | | | | |
| 175A | | | | | |
| 176A | | | | | |
| 176B | | | | | |

TABLE 4
Additional exemplary data of select compounds

| | GSK3α TR- | GSK3β TR- | GSK3α TR- | GSK3β TR- |
|---|---|---|---|---|
| | FRET 4 μM | FRET 2 μM | FRET 1 mM | FRET 1 mM |
| Compound | ATP GMean | ATP GMean | ATP GMean | ATP GMean |
| No. | IC₅₀ (μM) | IC₅₀ (μM) | IC₅₀ (μM) | IC₅₀ (μM) |
| 1002A | F | F | | |
| 1002B | C | C | | |
| 1004A | A | A | | |
| 1004B | C | F | | |
| 1006A | F | F | | |
| 1006B | F | F | | |
| 1006C | C | C | | |
| 1006D | B | B | | |
| 1007A | F | F | | |
| 1007B | A | A | C | D |
| 1008A | A | A | B | C |
| 1008B | C | C | | |
| 1009A | A | A | | |
| 1009B | B | B | | |
| 1009C | F | F | | |
| 1009D | F | F | | |
| 1010A | F | F | | |
| 1010B | A | A | | |
| 1011A | F | F | | |
| 1011B | A | A | | |
| 1012 | A | A | | |
| 1014A | C | F | | |
| 1014B | A | B | | |
| 1015A | A | A | | |
| 1015B | C | F | | |
| 1016A | A | A | | |
| 1016B | C | F | | |
| 1017A | A | B | | |
| 1017B | F | F | | |
| 1018A | F | F | | |
| 1018B | A | A | | |
| 1019A | A | A | B | C |
| 1019B | F | F | | |
| 1020A | C | F | | |
| 1020B | A | A | B | C |
| 102 | B | B | | |
| 102 A | A | A | C | D |
| 102 B | F | F | | |
| 1021A | F | F | | |
| 1021B | B | B | | |
| 1022A | B | B | | |
| 1022B | F | F | | |
| 1023A | A | A | B | C |
| 1023B | B | C | | |
| 1024A | C | F | | |
| 1024B | A | B | | |
| 1025A | A | A | | |
| 1025B | B | F | | |
| 1027A | F | F | | |
| 1027B | A | B | | |
| 1028A | C | F | | |
| 1028B | A | A | | |
| 149A | A | A | A | B |
| 149B | B | B | | |
| 150A | C | F | | |
| 150B | A | A | B | C |
| 1031A | B | B | | |
| 1031B | A | A | | |
| 1032A | A | B | | |
| 1032B | A | A | | |
| 1033A | C | F | | |
| 1033B | A | A | | |
| 1034A | F | F | | |
| 1034B | A | A | | |
| 1035A | C | C | | |
| 1035B | C | C | | |
| 154A | F | F | | |
| 154B | A | A | A | B |
| 1037A | C | C | | |
| 1037B | A | A | | |
| 1038A | A | A | | |
| 1038B | A | B | | |
| 1039A | B | B | | |
| 1039B | A | A | B | C |
| 161A | B | C | | |
| 161B | A | A | A | A |
| 165A | C | F | | |
| 165B | A | A | B | B |
| 110 A | A | A | | |
| 110 B | F | F | | |
| 114 | A | A | | |
| 114 A | A | A | B | C |
| 114 B | B | C | | |
| 133 A | F | F | | |
| 133 B | A | A | | |
| 147 A | C | F | | |
| 147 B | A | A | B | C |
| C1 | F | F | | |

TABLE 4
Additional exemplary data of select compounds

| | | | | MDCK |
|---|---|---|---|---|
| | Microsome | MDCK | MDCK | Permeability |
| | Stability (Human) | Permeability | Permeability | (MDR1-MDCK); |
| | (DMPK_BIIB); | (MDR1-MDCK); | (MDR1-MDCK); | Mean; P. |
| Compound | Mean; in vitro | Mean; Papp(A-B) | Mean; Papp(B-A) | Ratio(B-A/A-B) |
| No. | t_{1/2} (min) | (10⁻⁶ cm/s) | (10⁻⁶ cm/s) | Efflux Ratio |
| C1 | | | | |
| 102 | | | | |
| 102 A | 9.1 | 8.7 | 26 | 2.9 |
| 102 B | 1.2 | 6.7 | 29 | 4.3 |
| 1002A | | | | |
| 1002B | | | | |
| 110A | 5.4 | 22 | 31 | 1.4 |
| 110B | 1.7 | 13 | 27 | 2.1 |
| 1004A | 7.7 | 1.2 | 42 | 37 |
| 1004B | 5.3 | 0.7 | 47 | 65 |
| 114 | | | | |
| 114A | 37 | 10 | 20 | 2.0 |
| 114B | 1.7 | 12 | 24 | 2.1 |
| 1006A | | | | |
| 1006B | | | | |
| 1006C | 37 | 18 | 31 | 1.8 |
| 1006D | 14 | 19 | 28 | 1.5 |
| 1007A | | | | |
| 1007B | 211 | 7.8 | 45 | 5.7 |
| 1008A | 19 | 22 | 26 | 1.2 |
| 1008B | 1.6 | 21 | 31 | 1.5 |
| 1009A | 6.3 | 34 | 35 | 1.0 |
| 1009B | 11 | 32 | 47 | 1.5 |
| 1009C | 2.1 | 23 | 21 | 0.9 |
| 1009D | 1.6 | 23 | 28 | 1.3 |
| 1010A | 6.1 | 0.60 | 28 | 51 |
| 1010B | 11 | 0.80 | 39 | 48 |
| 1011A | | | | |
| 1011B | 3.8 | 15 | 28 | 1.9 |
| 1012 | | | | |
| 133A | 1.4 | 13 | 19 | 1.5 |
| 133B | 73 | 11 | 16 | 1.5 |
| 1014A | | | | |
| 1014B | | | | |
| 1015A | 2.8 | 4.7 | 44 | 9.5 |
| 1015B | | | | |
| 1016A | 1.5 | 4.5 | 32 | 6.9 |
| 1016B | | | | |
| 1017A | 6.2 | 6.9 | 32 | 4.6 |
| 1017B | | | | |
| 1018A | | | | |
| 1018B | | | | |
| 1019A | 4.8 | 4.2 | 39 | 9.5 |
| 1019B | | | | |
| 1020A | | | | |
| 1020B | 6.4 | 14 | 24 | 1.8 |
| 1021A | | | | |
| 1021B | | | | |
| 1022A | | | | |
| 1022B | | | | |
| 1023A | 3.2 | 3.0 | 27 | 9.0 |
| 1023B | | | | |
| 1024A | | | | |
| 1024B | 4.3 | 2.8 | 31 | 11 |
| 1025A | 5.5 | 4.6 | 42 | 9.1 |
| 1025B | | | | |
| 147 A | | | | |
| 147 B | 14 | 8.1 | 31 | 3.8 |
| 1027A | | | | |
| 1027B | 3.1 | 5.1 | 30 | 5.8 |
| 1028A | | | | |
| 1028B | | | | |
| 149A | 9.9 | 2.3 | 41 | 18 |
| 149B | | | | |
| 150A | | | | |
| 150B | 2.7 | 2.9 | 29 | 9.9 |
| 1031A | | | | |
| 1031B | 19 | 6.2 | 22 | 3.6 |
| 1032A | | | | |
| 1032B | 6.4 | 2.3 | 26 | 11 |
| 1033A | | | | |
| 1033B | | | | |
| 1034A | | | | |
| 1034B | 4.1 | 7.6 | 25 | 3.3 |
| 1035A | | | | |
| 1035B | | | | |
| 154A | | | | |
| 154B | 6.6 | 3.1 | 25 | 8.3 |
| 1037A | | | | |
| 1037B | 5.6 | 2.7 | 26 | 9.6 |
| 1038A | 16 | 0.90 | 41 | 43 |
| 1038B | 13 | 0.70 | 34 | 49 |
| 1039A | | | | |
| 1039B | 4.6 | 6.6 | 22 | 3.3 |
| 161A | | | | |
| 161B | 5.6 | 5.2 | 33 | 6.4 |
| 165A | | | | |
| 165B | 4.7 | 4.4 | 24 | 5.5 |

In Table 5, A: ≤0.10 μM; B: >0.10 to ≤1.0 μM; C: >1.0 to ≤5.0 μM; D: >5.0 to ≤20 μM; E: >20 to ≤60 μM; F: >5.0 μM; and G: >20 μM.

TABLE 5
Additional exemplary data of select compounds

| | GSK3α TR-FRET 1 mM | GSK3β TR-FRET 1 mM |
|---|---|---|
| Compound No. | ATP Average IC₅₀ (μM) | ATP Average IC₅₀ (μM) |
| 601A | C | C |
| 601B | D | D |
| 602A | | |
| 602B | | D |
| 603A | D | D |
| 603B | B | B |
| 604A | C | C |
| 604B | A | A |
| 605A | B | B |
| 605B | D | D |
| 606A | D | D |
| 606B | C | C |
| 607A | C | C |
| 607B | D | D |
| 608A | C | C |
| 608B | D | D |
| 609A | D | D |
| 609B | D | C |
| 610A | | |
| 610B | D | D |
| 611A | | |
| 611B | | |
| 612A | | D |
| 612B | D | D |
| 613A | | |
| 613B | D | C |
| 614A | D | D |
| 614B | B | B |
| 615A | C | C |
| 615B | A | A |
| 616A | B | B |
| 616B | B | B |
| 616C | D | D |
| 616D | D | D |
| 617A | D | D |
| 617B | B | B |
| 617C | D | D |
| 617D | B | B |
| 618A | D | D |
| 618B | B | B |
| 619A | D | D |
| 619B | D | D |
| 620A | D | D |
| 620B | D | D |
| 621A | D | D |
| 621B | | D |
| 622A | | D |
| 622B | D | D |
| 623A | D | |
| 623B | B | B |
| 624A | D | D |
| 624B | G | D |
| 625A | C | C |
| 625B | D | |
| 626A | D | D |
| 626B | D | C |
| 627A | B | B |
| 627B | D | C |
| 628A | C | C |
| 628B | C | B |
| 629A | | D |
| 629B | B | B |
| 630A | A | B |
| 630B | D | |
| 631A | D | D |
| 631B | D | D |
| 632A | C | B |
| 632B | B | B |
| 633A | D | C |
| 633B | C | B |
| 633C | D | D |
| 633D | C | B |
| 634A | D | D |
| 634B | A | A |
| 635A | D | D |
| 635B | | |
| 636A | C | B |
| 636B | D | C |
| 637A | | |
| 637B | | |
| 638A | A | B |
| 638B | D | D |
| 639A | D | D |
| 639B | A | A |
| 640A | | D |
| 640B | D | D |
| 641A | | |
| 641B | C | C |
| 642A | D | C |
| 642B | D | D |
| 643A | B | B |
| 643B | D | |
| 644A | B | B |
| 644B | | |
| 645A | D | D |
| 645B | D | C |
| 646A | D | D |
| 646B | C | B |
| 647A | B | B |
| 647B | D | D |
| 648A | D | C |
| 648B | C | B |
| 649A | D | C |
| 649B | D | D |
| 650A | D | C |
| 650B | B | B |
| 651A | D | D |
| 651B | B | B |
| 652A | D | D |
| 652B | A | D |
| 653A | C | C |
| 653B | A | A |
| 654A | | |
| 654B | | |
| 654C | | |
| 654D | | |
| 655A | | |
| 655B | | |
| 656A | B | B |
| 656B | A | A |
| 657A | D | D |
| 657B | D | C |

TABLE 6
Chemical structures of compounds having multiple chiral centers

| No. | Formula |
|---|---|
| 616A | 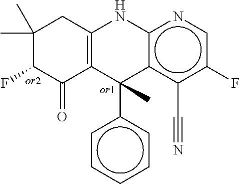 |
| 616B | 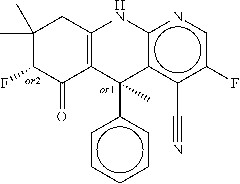 |
| 616C | 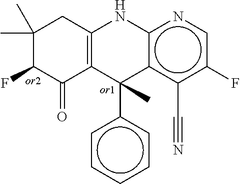 |
| 616D | 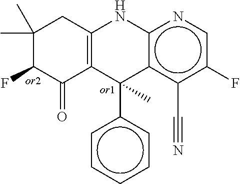 |
| 617A | 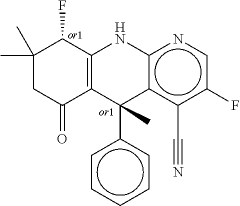 |
| 617B | 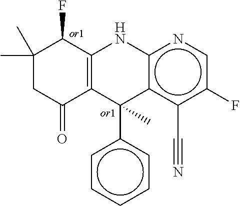 |
| 617C | 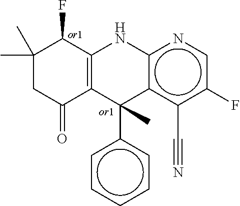 |
| 617D | 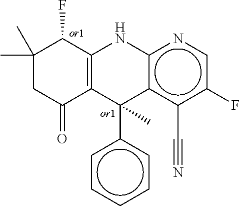 |
| 633A | 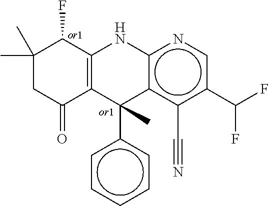 |
| 633B | 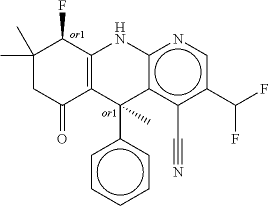 |
| 633C | 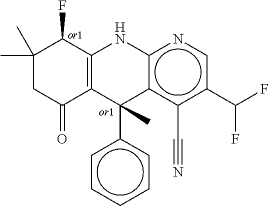 |
| 633D | 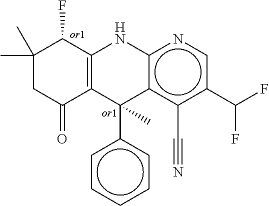 |
| 654A | 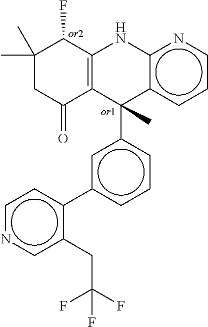 |
| 654B | 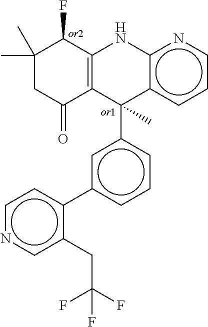 |
| 654C | 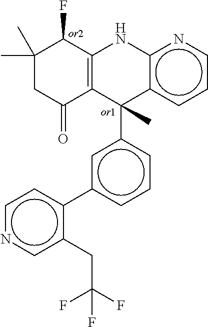 |
| 654D | 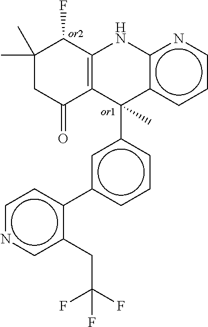 |
| 655A | 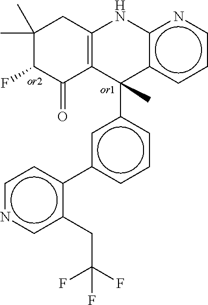 |
| 655B | 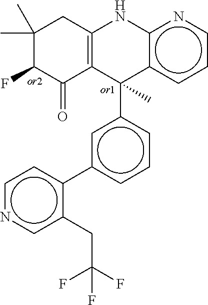 |

Compound C1 is of the formula:

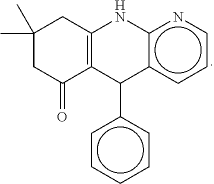

### Example 7. Co-Crystal Structure of GSK3, and Compound 133A

Compound 133A (100 mM stock in 100% DMSO) was added to 1 mg/mL human GSK3b (Protein buffer 50 mM Hepes pH 7.5, 150 mM NaCl, 2 mM DTT) with gentle mixing to achieve a final concentration of 0.11 mM 133A, resulting in a compound:protein ratio of 5:1; The GSK3b/compound complex was incubated at ice for 30 minutes and then concentrated up to 6.6 mg/mL using the 10 kDa cut-off Centricon concentrator. This complex was crystallized using the sitting drop vapor diffusion method at room temperature. Each drop contained 0.2 μL of protein plus 0.2 μL of reservoir. Reservoir solution: 0.1M Hepes pH 7.5, 20% w/v PEG 4,000, 10% isopropanol. Sitting drops were equilibrated against combining 40 μL of reservoir solution, and incubated at 20° C. Protein crystals grew to >100 μm after incubation at 20° C. for ten days. X-ray diffraction data were measured at beamline 17.ID.1 at the National Synchrotron Light Source II (NSLS-II), Brookhaven National Laboratory in Upton, NY, USA. All data were collected at a single wavelength of 0.920105 Å using a Dectris Eiger 9M detector.

TABLE 5
Summary of X-ray diffraction data

| X-ray beam wavelength | 0.920105 Å | |
|---|---|---|
| Space group | C 2 | |
| Unit cell | a = 106.590 Å, b = 85.311 Å, c = 113.300 Å | |
| | α = 90.00, γ = 95.68, β = 90.00 | |
| Resolution | 46.26-2.92 Å | (3.09-2.92 Å) |
| Measured reflections | 86,457 | (13,984) |
| Unique reflections | 22,190 | (3,588) |
| Rsym (%) | 0.11 | (0.98) |
| Completeness | 98.8 | (99.6) % |
| I/σ | 7.1 | (1.2) |
| Redundancy | 3.9 | (3.9) |
| Mn (I) half-set | 99.6 | (45.5) |
| correlation CC(1/2) | | |

| | |
|---|---|
| Molecules per | 2 |
| asymmetric unit | |

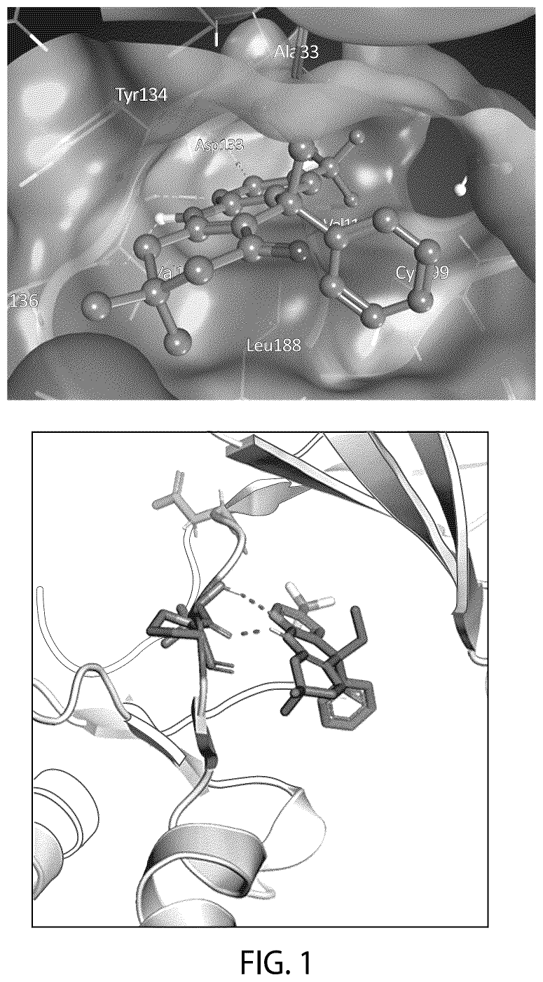

Additional exemplary results are shown in FIG. 1. FIG. 1 shows that Compound 133A is the same as Compound 133S.

### EQUIVALENTS AND SCOPE

In the claims and throughout, articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following claims.

What is claimed is:

1. A compound, or a pharmaceutically acceptable salt, solvate, hydrate, polymorph, co-crystal, tautomer, stereoisomer, isotopically labeled compound, or prodrug thereof, wherein the compound is of Formula I: 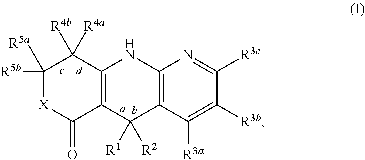
wherein:
R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R² is optionally substituted alkyl, optionally substituted alkenyl, or optionally substituted alkynyl;
or R¹ and R² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
each of R^{3a} and R^{3b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NRAS(═O)R^{A}, —NRAS(═O)OR^{A}, —NRAS(═O)SR^{A}, —NRAS(═O)N(R^{A})₂, —NRAS(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃;
or R^{3a} and R^{3b} are taken together with their intervening atoms to form an optionally substituted, monocyclic, carbocyclic, heterocyclic, or heteroaryl ring, or an optionally substituted phenyl ring;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{3a} is hydrogen;
X is 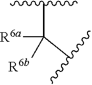
or —N(R)— each of R^{4a}, R^{4b}, R^{5a}, R^{5b}, R^{6a}, and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
or R^{4a} and R^{4b} are taken together with their intervening atom to form an optionally substituted, monocyclic, carbocyclic or heterocyclic ring;
or R^{4b} and R^{5a} are taken together with their intervening atoms to form an optionally substituted, monocyclic, carbocyclic or heterocyclic ring;
or R^{5a} and R^{5b} are taken together with their intervening atom to form an optionally substituted, monocyclic, carbocyclic or heterocyclic ring;
or R^{5b} and R^{6a} are taken together with their intervening atoms to form an optionally substituted, monocyclic, carbocyclic or heterocyclic ring;
or R^{6a} and R^{6b} are taken together with their intervening atom to form an optionally substituted, monocyclic, carbocyclic or heterocyclic ring; and
R⁶ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

2. The compound of claim 1, or a pharmaceutically acceptable salt, solvate, hydrate, polymorph, co-crystal, tautomer, stereoisomer, isotopically labeled compound, or prodrug thereof, wherein R¹ is optionally substituted aryl.

3. The compound of claim 1, or a pharmaceutically acceptable salt, solvate, hydrate, polymorph, co-crystal, tautomer, stereoisomer, isotopically labeled compound, or prodrug thereof, wherein the compound is of Formula I-1:

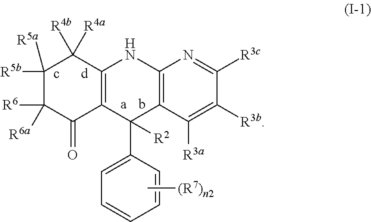

4. The compound of claim 1, or a pharmaceutically acceptable salt, solvate, hydrate, polymorph, co-crystal, tautomer, stereoisomer, isotopically labeled compound, or prodrug thereof, wherein the compound is of Formula I-2:

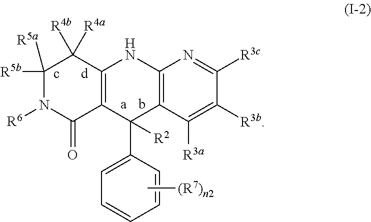

5. The compound of claim 1, or a pharmaceutically acceptable salt, solvate, hydrate, polymorph, co-crystal, tautomer, stereoisomer, isotopically labeled compound, or prodrug thereof, wherein the compound is of Formula I-3:

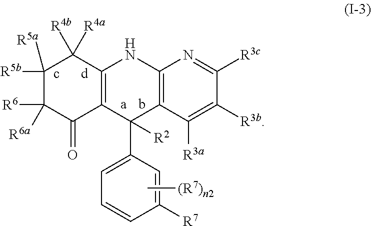

6. The compound of claim 1, or a pharmaceutically acceptable salt, solvate, hydrate, polymorph, co-crystal, tautomer, stereoisomer, isotopically labeled compound, or prodrug thereof, wherein the compound is of Formula I-4:

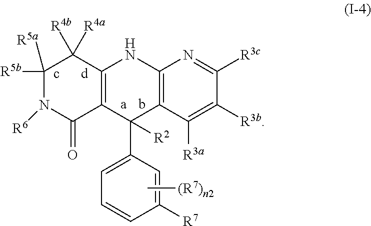

7. The compound of any one of claims 3-6, or a pharmaceutically acceptable salt, solvate, hydrate, polymorph, co-crystal, tautomer, stereoisomer, isotopically labeled compound, or prodrug thereof, wherein at least one instance of R⁷ is optionally substituted heteroaryl, optionally at least one instance of R⁷ is optionally substituted pyridinyl.

8. The compound of any one of claims 1-7, or a pharmaceutically acceptable salt, solvate, hydrate, polymorph, co-crystal, tautomer, stereoisomer, isotopically labeled compound, or prodrug thereof, wherein R² is optionally substituted alkyl.

9. The compound of any one of claims 1-8, or a pharmaceutically acceptable salt, solvate, hydrate, polymorph, co-crystal, tautomer, stereoisomer, isotopically labeled compound, or prodrug thereof, wherein R^{3a} is halogen, optionally substituted alkyl, optionally substituted alkenyl, optionally substituted carbocyclyl, or —CN.

10. The compound of any one of claims 1-9, or a pharmaceutically acceptable salt, solvate, hydrate, polymorph, co-crystal, tautomer, stereoisomer, isotopically labeled compound, or prodrug thereof, wherein R^{3b} is halogen, optionally substituted alkyl, optionally substituted carbocyclyl, or —CN.

11. The compound of any one of claims 1-10, or a pharmaceutically acceptable salt, solvate, hydrate, polymorph, co-crystal, tautomer, stereoisomer, isotopically labeled compound, or prodrug thereof, wherein each of R^{4a} and R^{4b} is independently hydrogen, halogen, or optionally substituted alkyl.

12. The compound of any one of claims 1-11, or a pharmaceutically acceptable salt, solvate, hydrate, polymorph, co-crystal, tautomer, stereoisomer, isotopically labeled compound, or prodrug thereof, wherein R^{5a} is —CH₃.

13. The compound of any one of claims 1-12, or a pharmaceutically acceptable salt, solvate, hydrate, polymorph, co-crystal, tautomer, stereoisomer, isotopically labeled compound, or prodrug thereof, wherein R^{5b} is —CH₃.

14. The compound of any one of claims 1-3, 5, and 7-13, or a pharmaceutically acceptable salt, solvate, hydrate, polymorph, co-crystal, tautomer, stereoisomer, isotopically labeled compound, or prodrug thereof, wherein each of R^{6a} and R^{6b} is independently hydrogen, halogen, or optionally substituted alkyl.

15. The compound of claim 1, or a pharmaceutically acceptable salt, solvate, hydrate, polymorph, co-crystal, tautomer, stereoisomer, isotopically labeled compound, or prodrug thereof, wherein the compound is of any one of the formulae:

| No. | Formula |
|---|---|
| 101R | 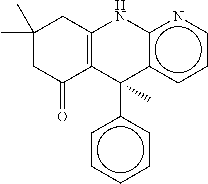 |
| 101S | 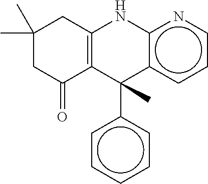 |
| 102R | 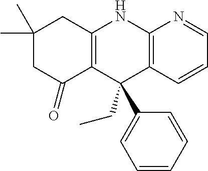 [SMILES: CC[C@]1(c2ccccc2)C2=C(CC(C)(C)CC2=O)Nc2ncccc21] |
| 102S | 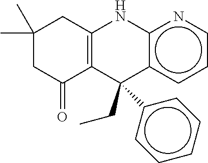 |
| 103R | 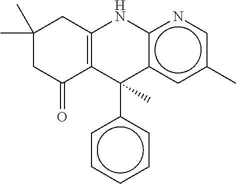 |
| 103S | 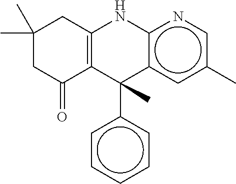 |
| 104R | 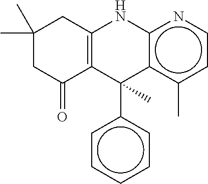 |
| 104S | 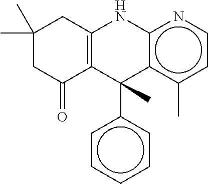 |
| 105R | 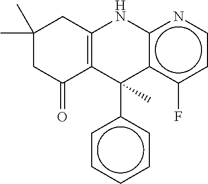 |
| 105S | 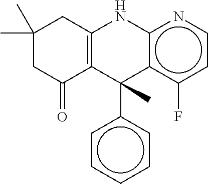 |
| 107R | 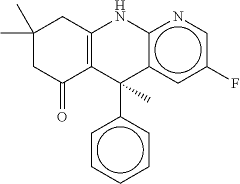 |
| 107S | 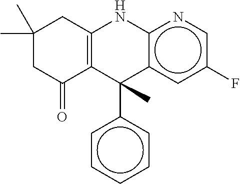 |
| 108R | 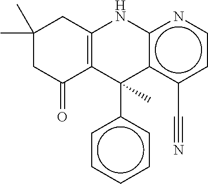 |
| 108S | 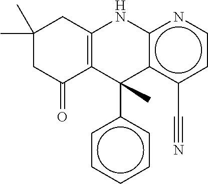 |
| 109R | 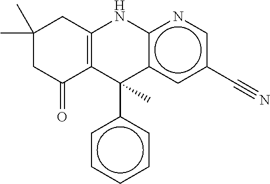 |
| 109S | 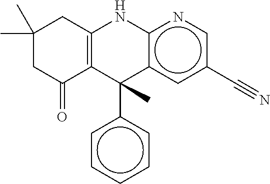 |
| 110R | 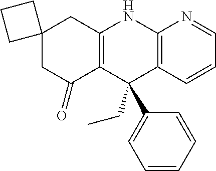 [SMILES: CC[C@]1(c2ccccc2)C2=C(CC3(CCC3)CC2=O)Nc2ncccc21] |
| 110S | 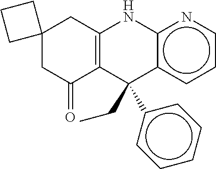 |
| 111R | 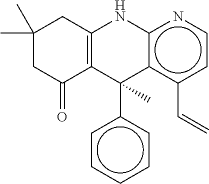 |
| 111S | 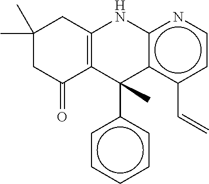 |
| 112R | 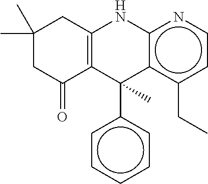 |
| 112S | 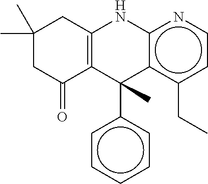 |
| 113R | 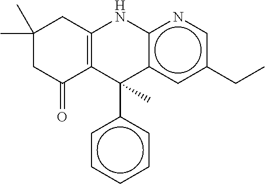 |
| 113S | 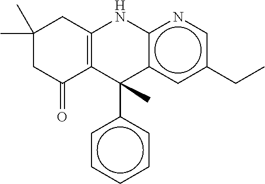 |
| 114R | 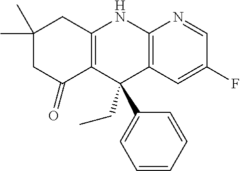 [SMILES: CC[C@]1(c2ccccc2)C2=C(CC(C)(C)CC2=O)Nc2ncc(F)cc21] |
| 114S | 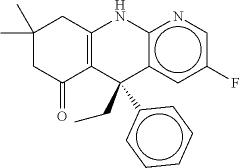 |
| 115R | 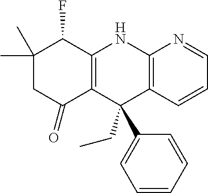 [SMILES: CC[C@]1(c2ccccc2)C2=C(Nc3ncccc31)[C@@H](F)C(C)(C)CC2=O] |
| 115S | 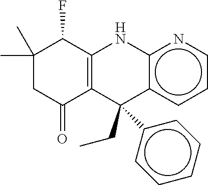 |
| 116R | 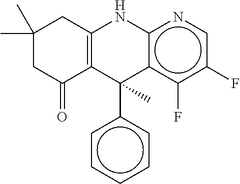 |
| 116S | 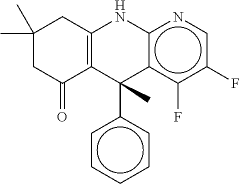 |
| 117R | 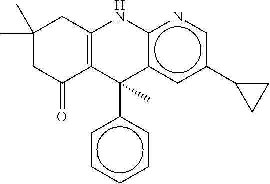 |
| 117S | 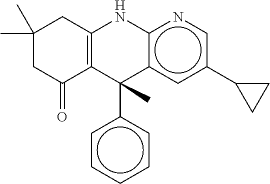 |
| 118R | 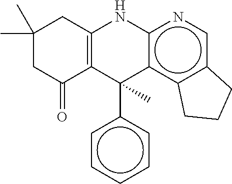 |
| 118S | 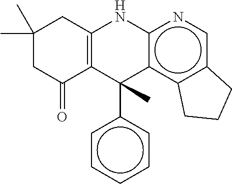 |
| 119R | 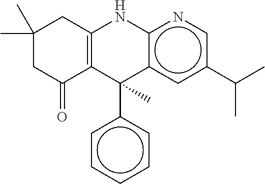 |
| 119S | 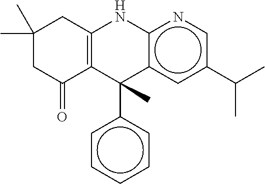 |
| 120R | 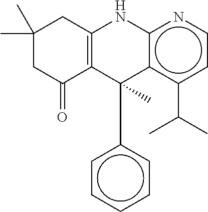 |
| 120S | 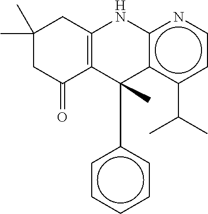 |
| 121R | 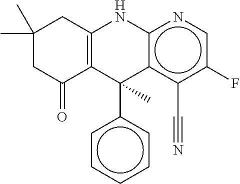 |
| 121S | 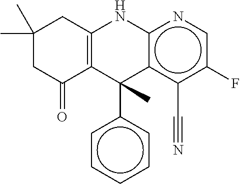 |
| 122R | 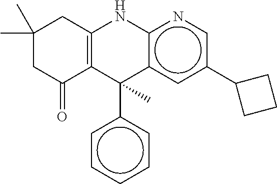 |
| 122S | 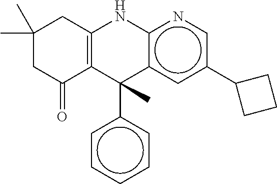 |
| 123R | 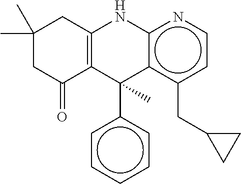 |
| 123S | 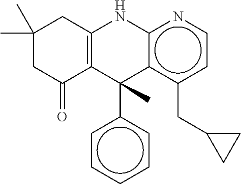 |
| 124R | 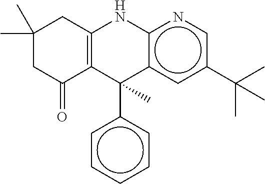 |
| 124S | 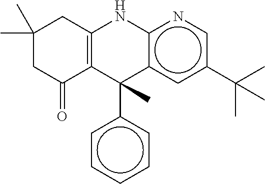 |
| 125R | 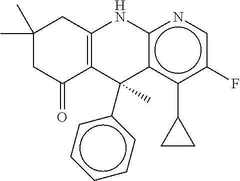 |
| 125S | 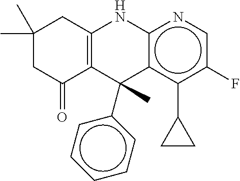 |
| 126R | 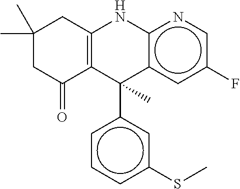 |
| 126S | 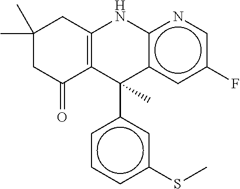 |
| 127R | 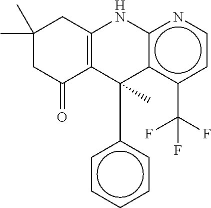 |
| 127S | 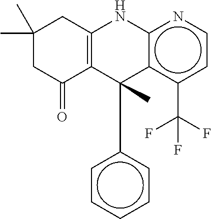 |
| 128R | 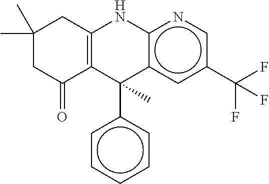 |
| 128S | 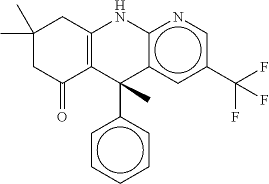 |
| 129R | 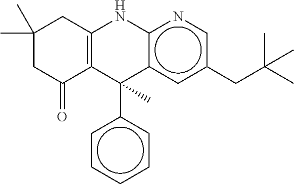 |
| 129S | 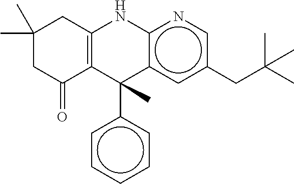 |
| 130R | 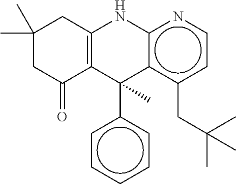 |
| 130S | 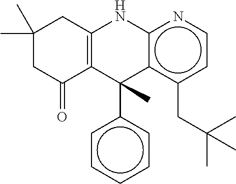 |
| 131R | 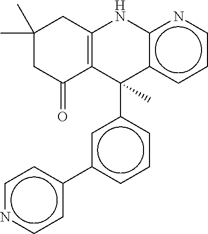 |
| 131S | 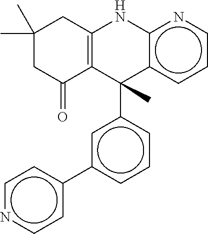 |
| 132R | 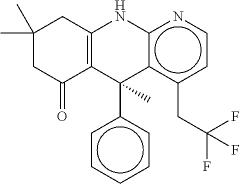 |
| 132S | 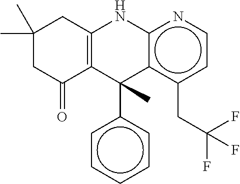 |
| 133R | 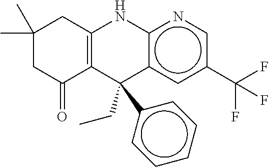 |
| 133S | 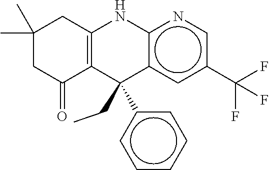 |
| 134R | 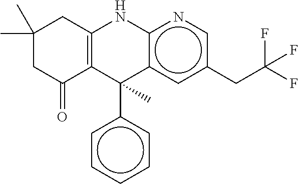 |
| 134S | 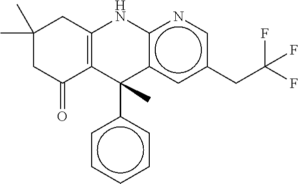 |
| 135R | 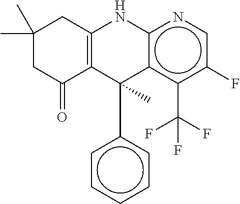 |
| 135S | 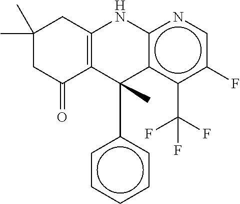 |
| 136R | 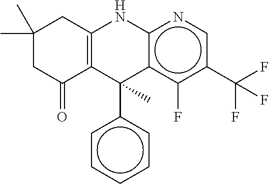 |
| 136S | 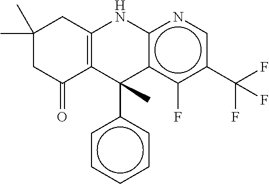 |
| 137R | 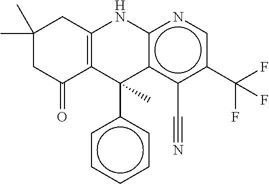 |
| 137S | 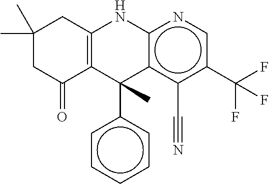 |
| 138R | 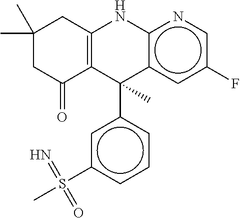 |
| 138S | 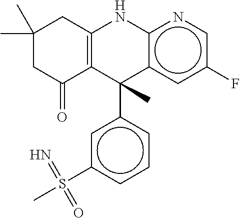 |
| 139R | 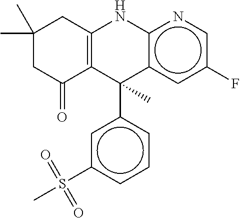 |
| 139S | 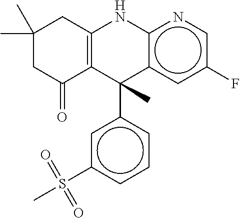 |
| 140R | 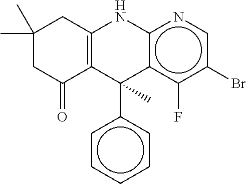 |
| 140S | 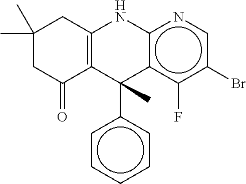 |
| 141R | 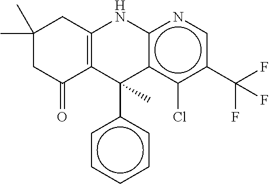 |
| 141S | 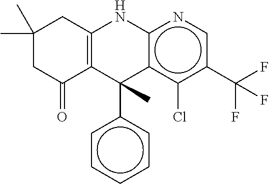 |
| 142R | 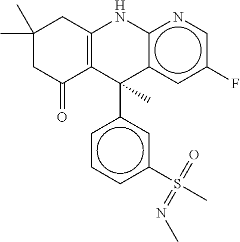 |
| 142S | 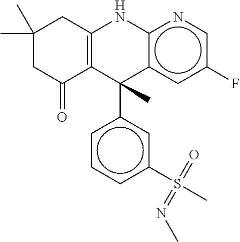 |
| 143R | 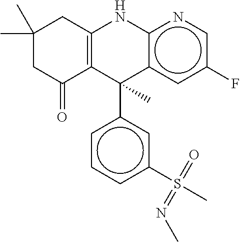 |
| 143S | 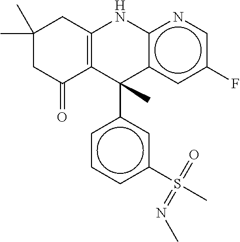 |
| 144R | 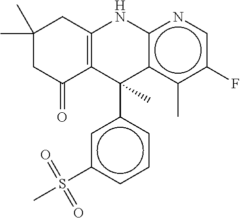 |
| 144S | 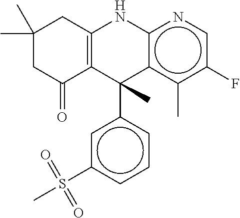 |
| 145R | 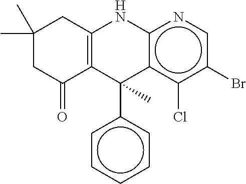 |
| 145S | 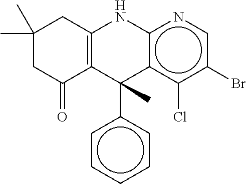 |
| 146R | 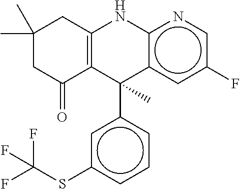 |
| 146S | 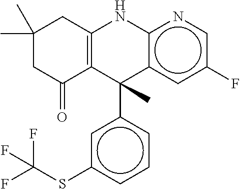 |
| 147R | 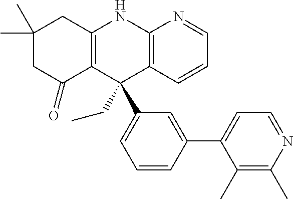 |
| 147S | 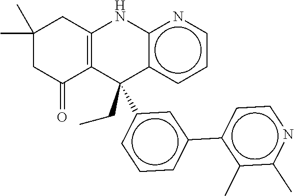 |
| 148R | 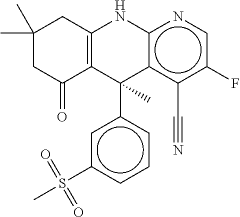 |
| 148S | 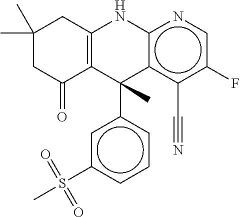 |
| 149R | 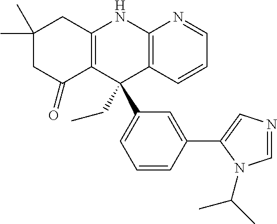 [SMILES: CC[C@]1(c2cccc(-c3cncn3C(C)C)c2)C2=C(CC(C)(C)CC2=O)Nc2ncccc21] |
| 149S | 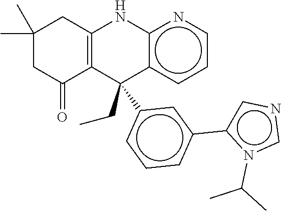 |
| 150R | 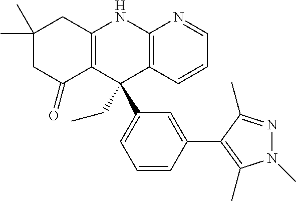 |
| 150S | 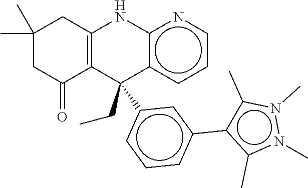 |
| 151R | 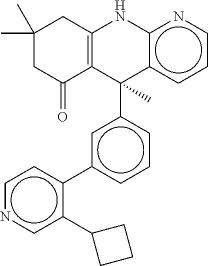 |
| 151S | 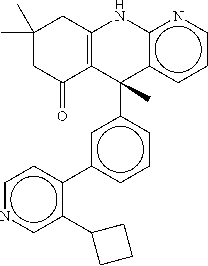 |
| 152R | 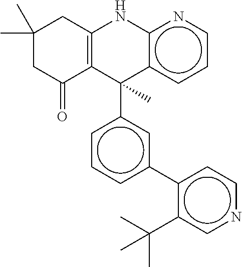 |
| 152S | 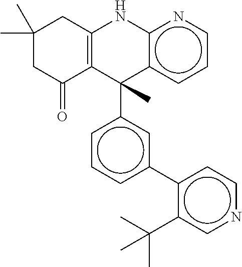 |
| 153R | 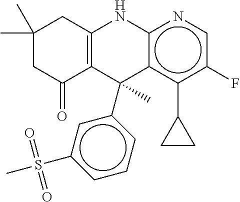 |
| 153S | 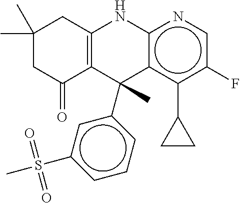 |
| 154R | 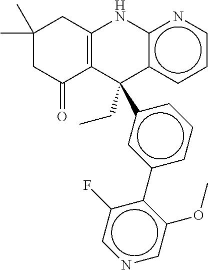 |
| 154S | 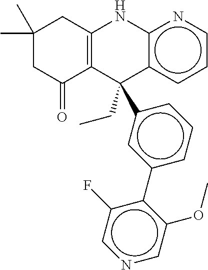 |
| 155R | 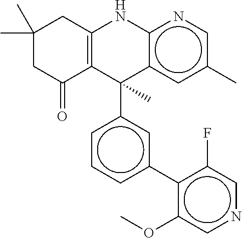 |
| 155S | 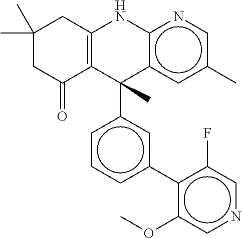 |
| 156R | 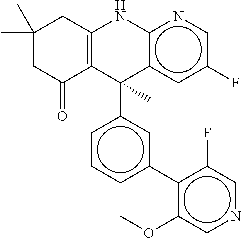 |
| 156S | 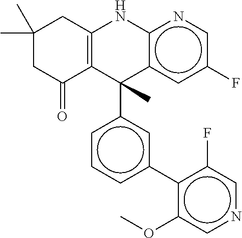 |
| 157R | 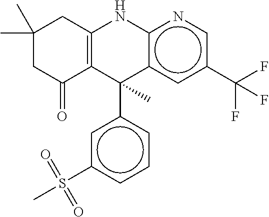 |
| 157S | 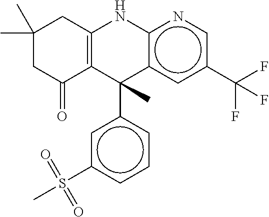 |
| 158R | 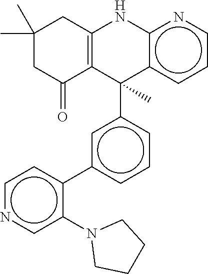 |
| 158S | 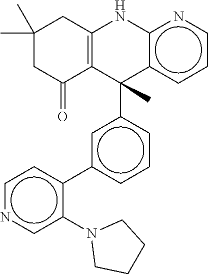 |
| 159R | 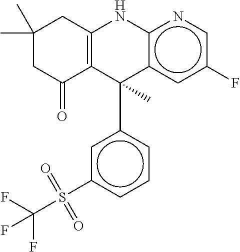 |
| 159S | 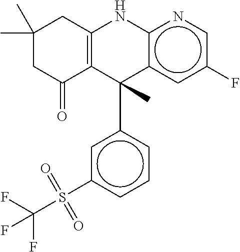 |
| 160R | 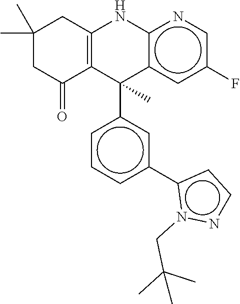 |
| 160S | 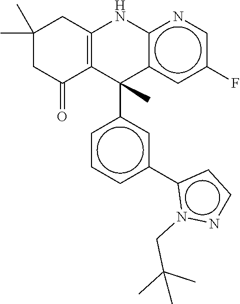 |
| 161R | 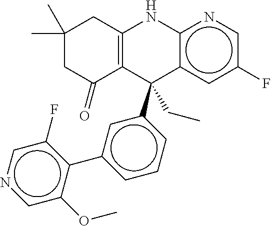 |
| 161S | 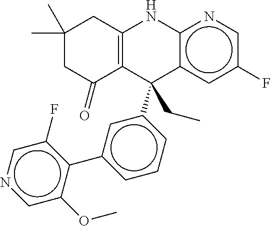 |
| 162R | 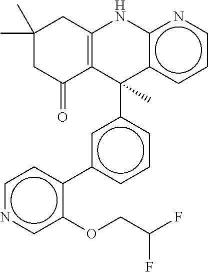 |
| 162S | 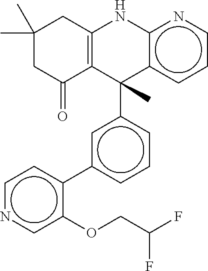 |
| 163R | 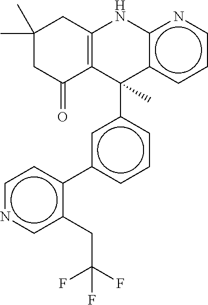 |
| 163S | 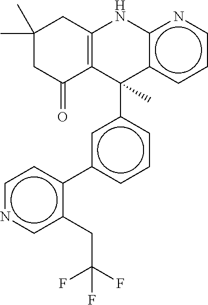 |
| 164R | 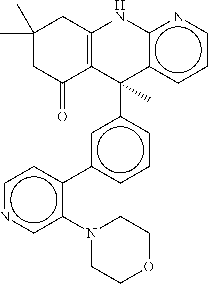 |
| 164S | 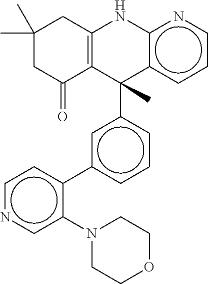 |
| 165R | 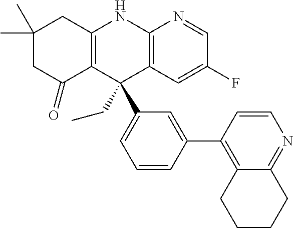 [SMILES: CC[C@]1(c2cccc(-c3ccnc4c3CCCC4)c2)C2=C(CC(C)(C)CC2=O)Nc2ncc(F)cc21] |
| 165S | 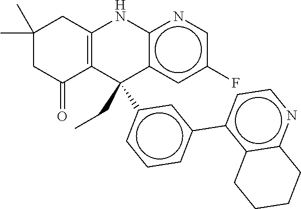 |
| 166R | 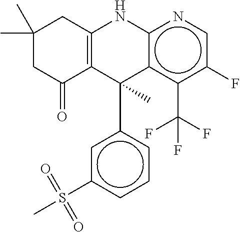 |
| 166S | 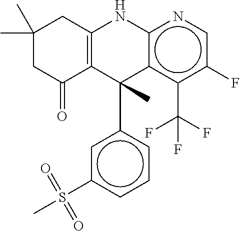 |
| 167R | 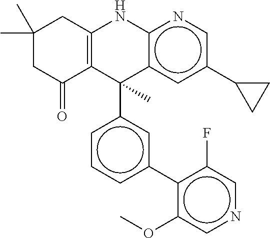 |
| 167S | 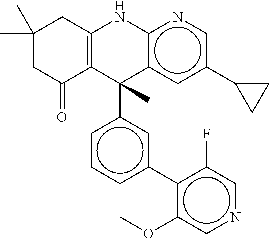 |
| 168R | 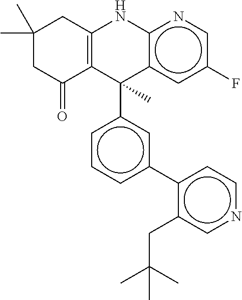 |
| 168S | 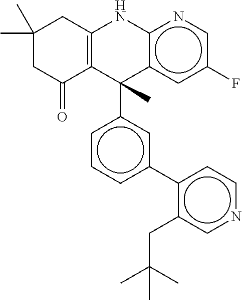 |
| 169R | 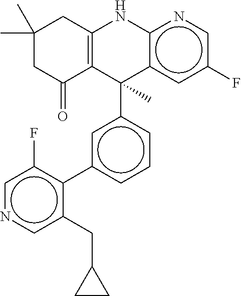 |
| 169S | 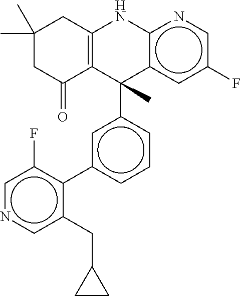 |
| 170R | 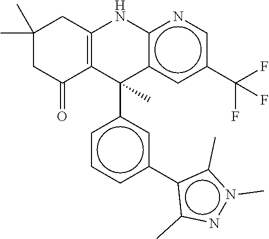 |
| 170S | 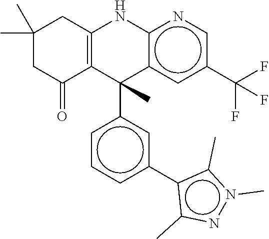 |
| 171R | 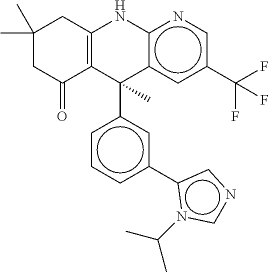 |
| 171S | 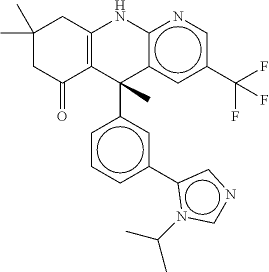 |
| 172R | 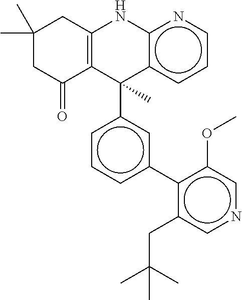 |
| 172S | 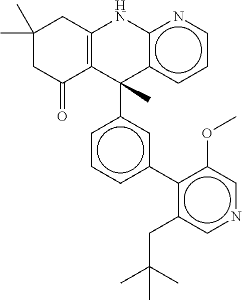 |
| 173R | 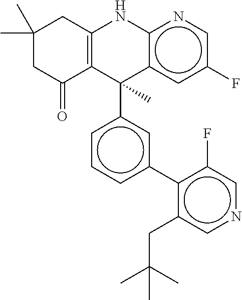 |
| 173S | 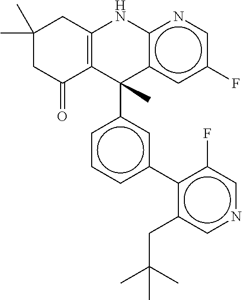 |
| 174R | 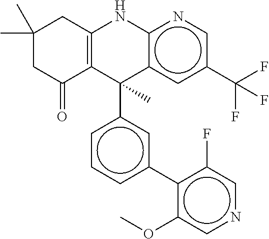 |
| 174S | 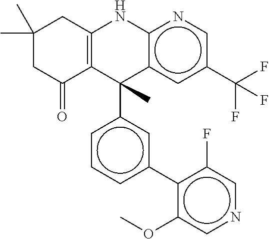 |
| 175R | 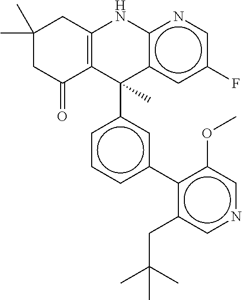 |
| 175S | 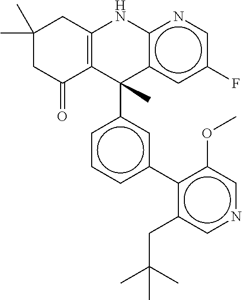 |
| 176R | 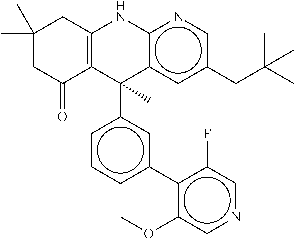 |
| 176S | 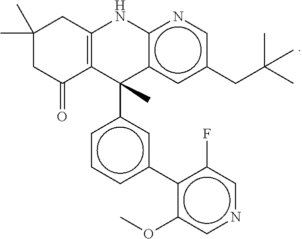 |

16. The compound of claim 1, or a pharmaceutically acceptable salt, solvate, hydrate, polymorph, co-crystal, tautomer, stereoisomer, isotopically labeled compound, or prodrug thereof, wherein the compound is of any one of the formulae:

| No. | Formula |
|---|---|
| 501R | 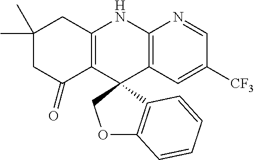 [SMILES: CC1(C)CC(=O)C2=C(C1)Nc1ncc(C(F)(F)F)cc1[C@@]21COc2ccccc21] |
| 501S | 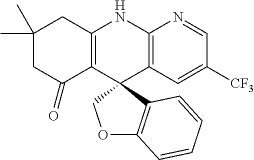 [SMILES: CC1(C)CC(=O)C2=C(C1)Nc1ncc(C(F)(F)F)cc1[C@]21COc2ccccc21] |
| 502R | 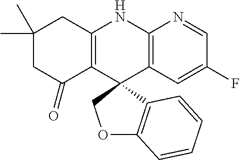 [SMILES: CC1(C)CC(=O)C2=C(C1)Nc1ncc(F)cc1[C@@]21COc2ccccc21] |
| 502S | 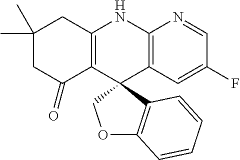 [SMILES: CC1(C)CC(=O)C2=C(C1)Nc1ncc(F)cc1[C@]21COc2ccccc21] |
| 503R | 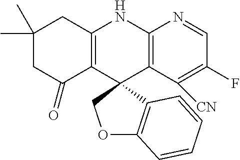 [SMILES: CC1(C)CC(=O)C2=C(C1)Nc1ncc(F)c(C#N)c1[C@@]21COc2ccccc21] |
| 503S | 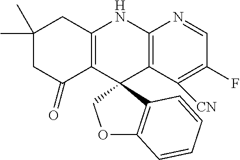 [SMILES: CC1(C)CC(=O)C2=C(C1)Nc1ncc(F)c(C#N)c1[C@]21COc2ccccc21] |
| 504R | 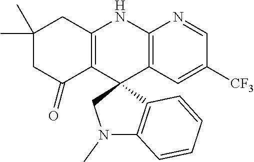 [SMILES: CN1C[C@]2(C3=C(CC(C)(C)CC3=O)Nc3ncc(C(F)(F)F)cc32)c2ccccc21] |
| 504S | 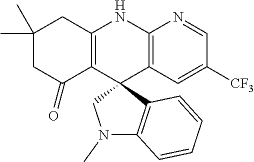 [SMILES: CN1C[C@@]2(C3=C(CC(C)(C)CC3=O)Nc3ncc(C(F)(F)F)cc32)c2ccccc21] |
| 505R | 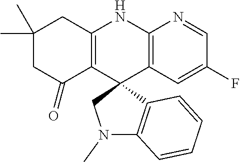 [SMILES: CN1C[C@]2(C3=C(CC(C)(C)CC3=O)Nc3ncc(F)cc32)c2ccccc21] |
| 505S | 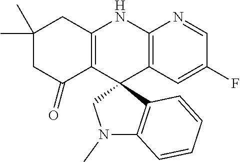 [SMILES: CN1C[C@@]2(C3=C(CC(C)(C)CC3=O)Nc3ncc(F)cc32)c2ccccc21] |
| 506R | 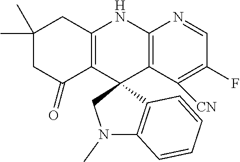 [SMILES: CN1C[C@]2(C3=C(CC(C)(C)CC3=O)Nc3ncc(F)c(C#N)c32)c2ccccc21] |
| 506S | 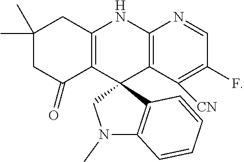 [SMILES: CN1C[C@@]2(C3=C(CC(C)(C)CC3=O)Nc3ncc(F)c(C#N)c32)c2ccccc21] |

17. The compound of claim 1, or a pharmaceutically acceptable salt, solvate, hydrate, polymorph, co-crystal, tautomer, stereoisomer, isotopically labeled compound, or prodrug thereof, wherein the compound is of any one of the formulae:

| No. | Formula |
|---|---|
| 601R | 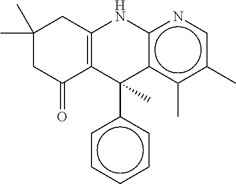 |
| 601S | 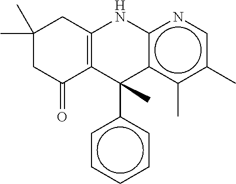 |
| 602R | 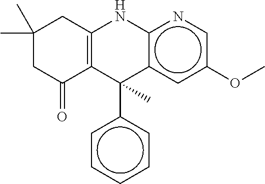 |
| 602S | 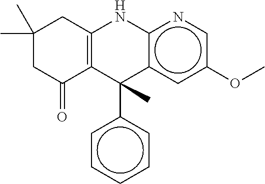 |
| 603R | 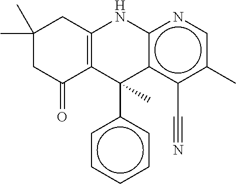 |
| 603S | 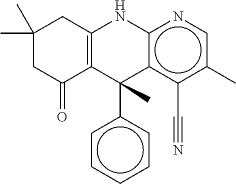 |
| 604R | 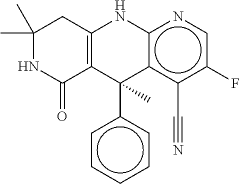 |
| 604S | 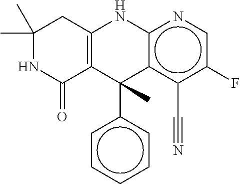 |
| 605R | 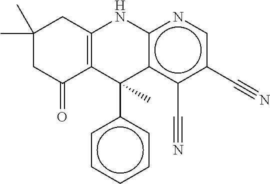 |
| 605S | 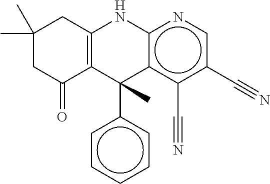 |
| 606R | 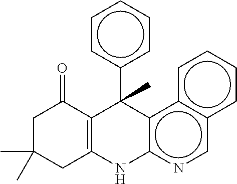 |
| 606S | 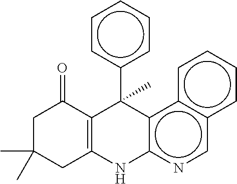 |
| 607R | 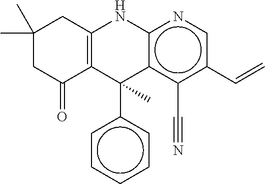 |
| 607S | 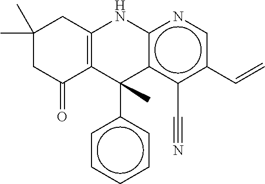 |
| 608R | 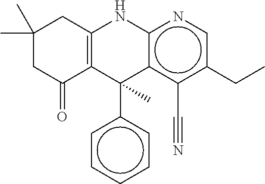 |
| 608S | 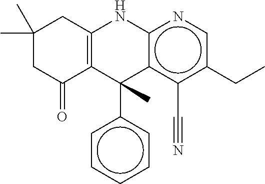 |
| 609R | 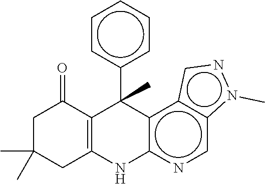 |
| 609S | 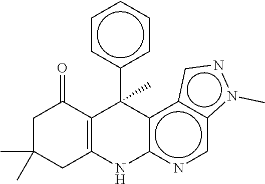 |
| 610R | 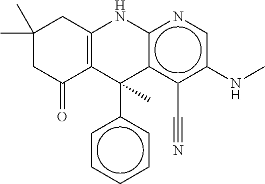 |
| 610S | 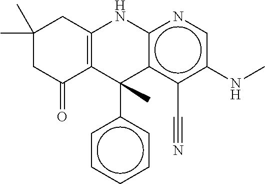 |
| 611R | 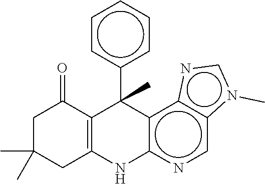 |
| 611S | 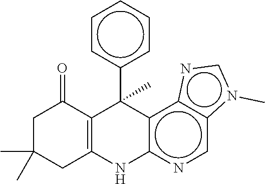 |
| 612R | 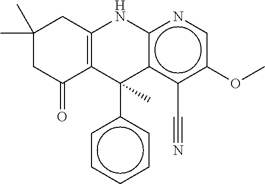 |
| 612S | 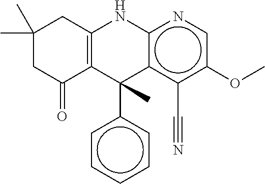 |
| 613R | 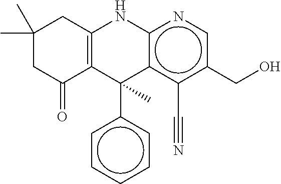 |
| 613S | 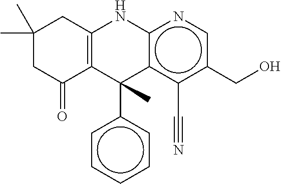 |
| 614R | 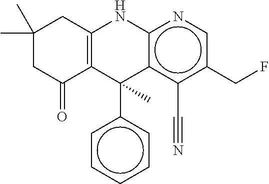 |
| 614S | 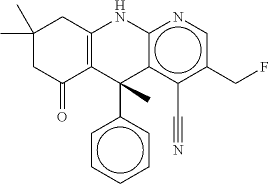 |
| 615R | 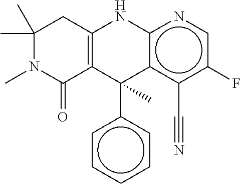 |
| 615S | 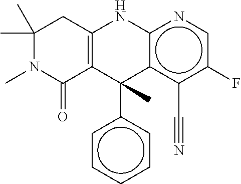 |
| 616RR | 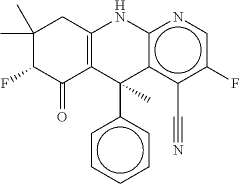 |
| 616SR | 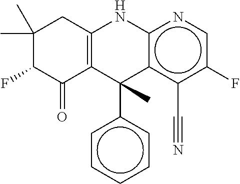 |
| 616RS | 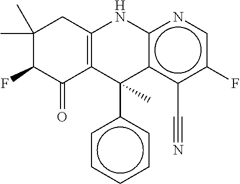 |
| 616SS | 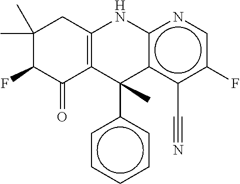 |
| 617RR | 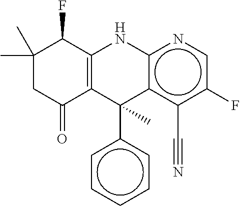 |
| 617SR | 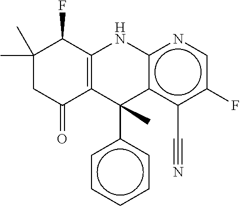 |
| 617RS | 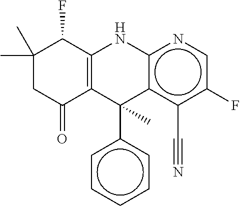 |
| 617SS | 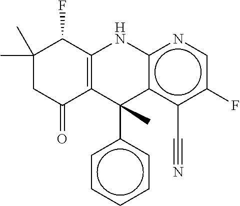 |
| 618R | 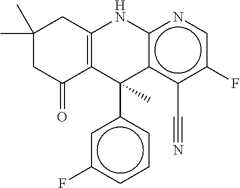 |
| 618S | 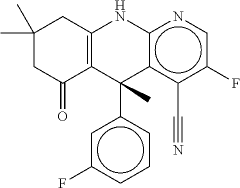 |
| 619R | 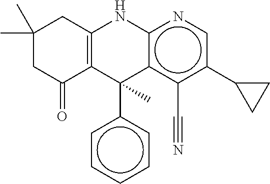 |
| 619S | 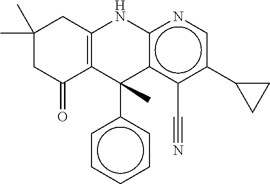 |
| 620R | 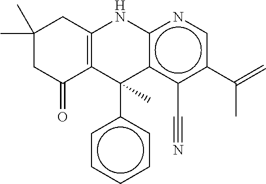 |
| 620S | 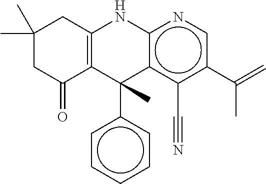 |
| 621R | 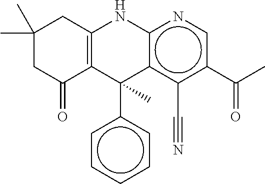 |
| 621S | 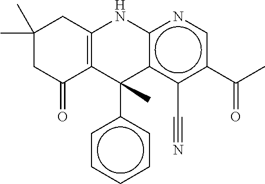 |
| 622R | 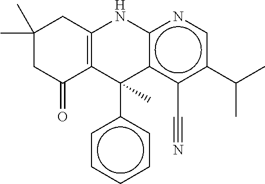 |
| 622S | 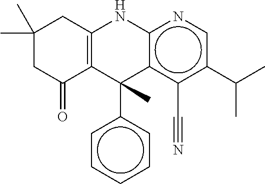 |
| 623R | 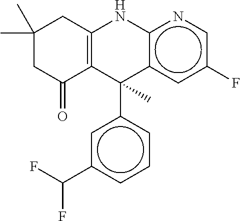 |
| 623S | 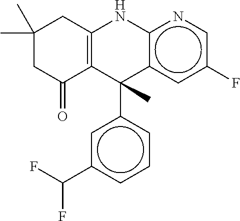 |
| 624R | 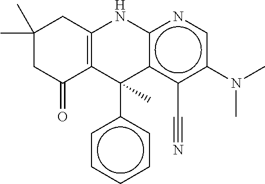 |
| 624S | 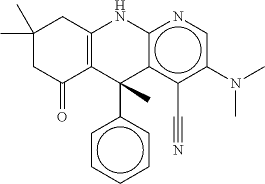 |
| 625R | 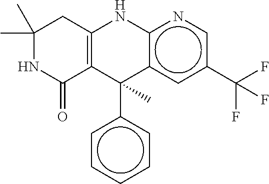 |
| 625S | 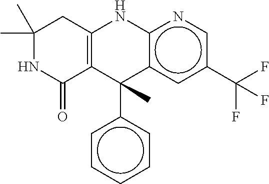 |
| 626R | 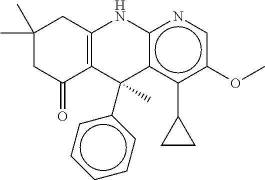 |
| 626S | 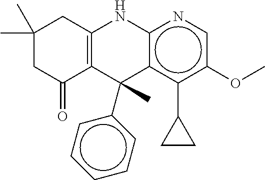 |
| 627R | 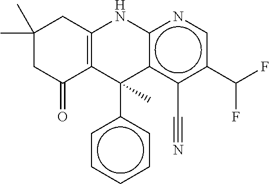 |
| 627S | 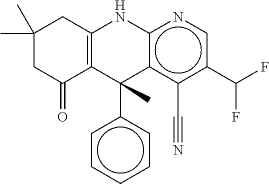 |
| 628R | 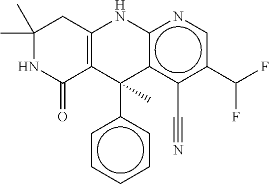 |
| 628S | 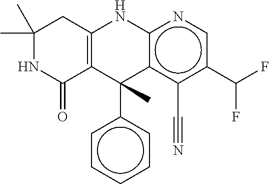 |
| 629R | 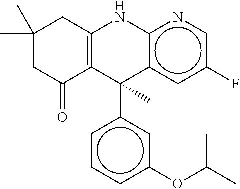 |
| 629S | 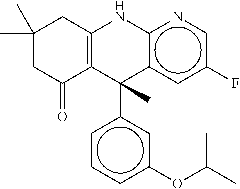 |
| 630R | 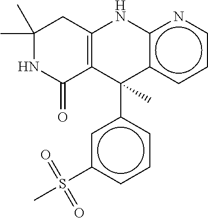 |
| 630S | 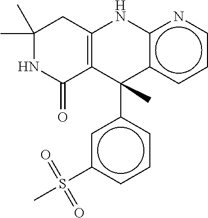 |
| 631R | 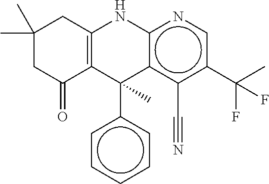 |
| 631S | 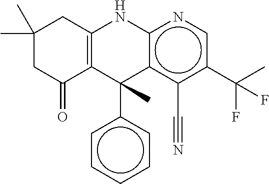 |
| 632R | 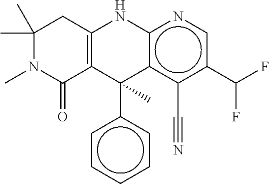 |
| 632S | 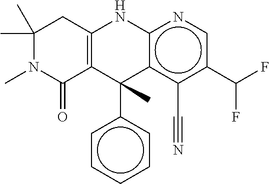 |
| 633RR | 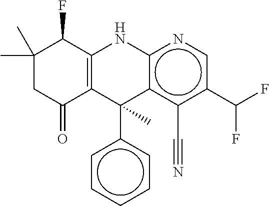 |
| 633SR | 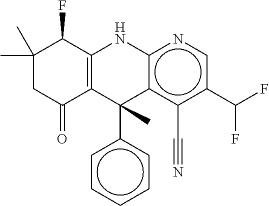 |
| 633RS | 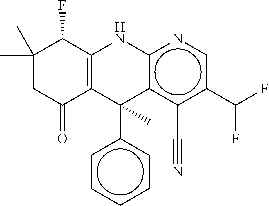 |
| 633SS | 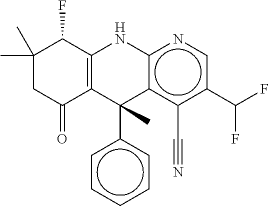 |
| 634R | 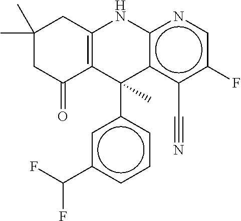 |
| 634S | 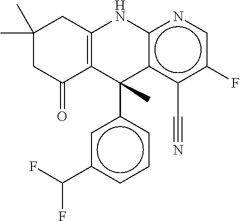 |
| 635R | 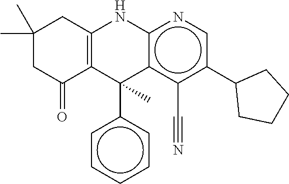 |
| 635S | 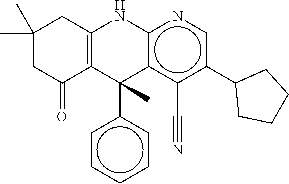 |
| 636R | 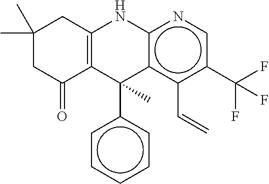 |
| 636S | 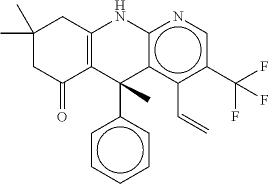 |
| 637R | 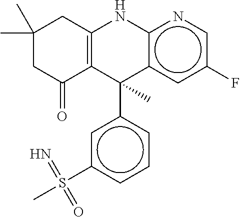 |
| 637S | 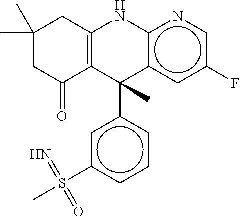 |
| 638R | 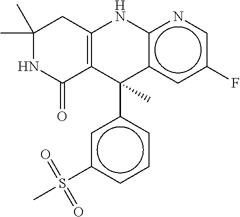 |
| 638S | 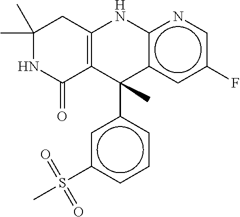 |
| 639R | 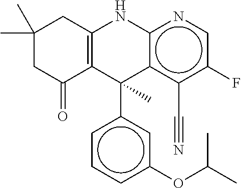 |
| 639S | 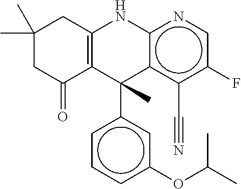 |
| 640R | 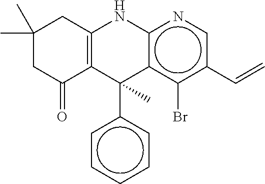 |
| 640S | 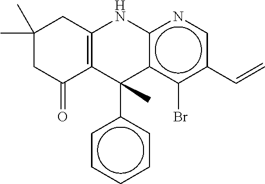 |
| 641R | 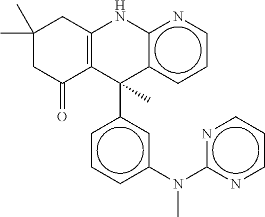 |
| 641S | 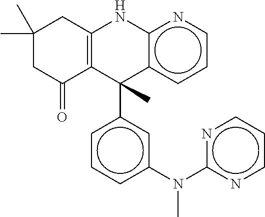 |
| 642R | 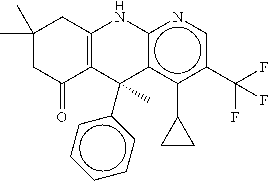 |
| 642S | 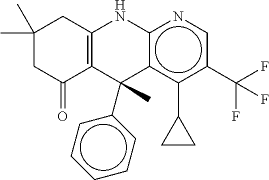 |
| 643R | 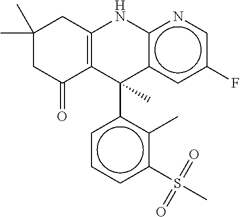 |
| 643S | 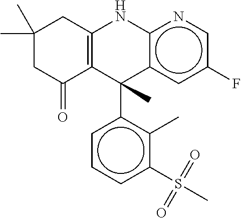 |
| 644R | 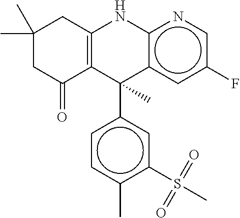 |
| 644S | 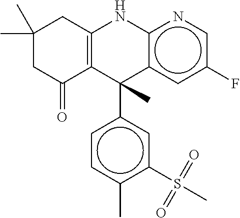 |
| 645R | 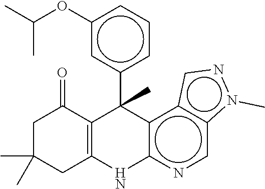 |
| 645S | 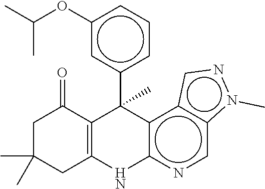 |
| 646R | 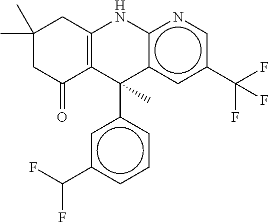 |
| 646S | 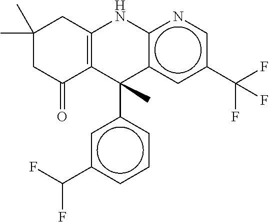 |
| 647R | 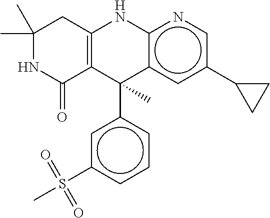 |
| 647S | 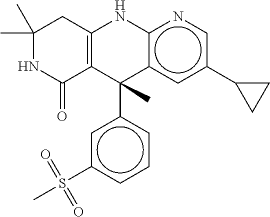 |
| 648R | 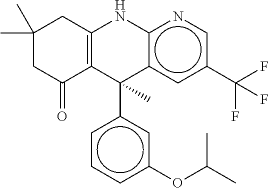 |
| 648S | 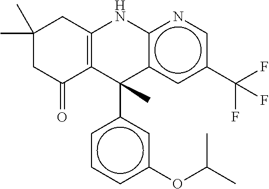 |
| 649R | 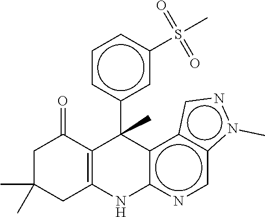 |
| 649S | 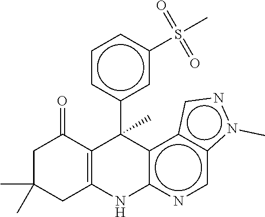 |
| 650R | 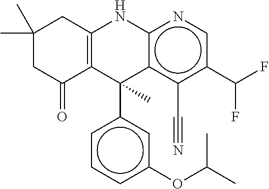 |
| 650S | 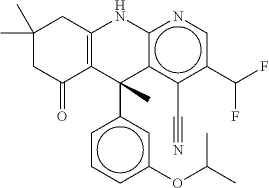 |
| 651R | 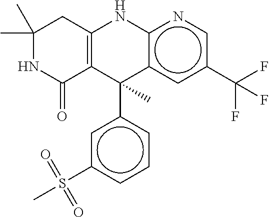 |
| 651S | 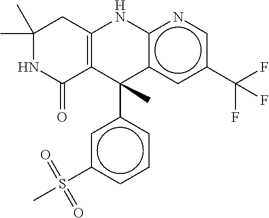 |
| 652R | 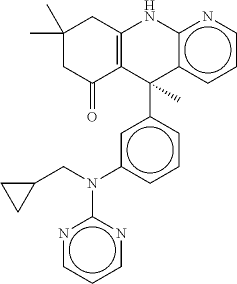 |
| 652S | 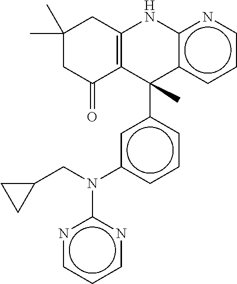 |
| 653R | 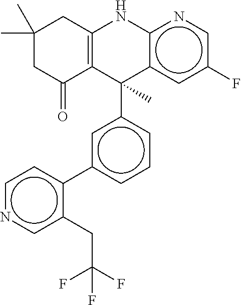 |
| 653S | 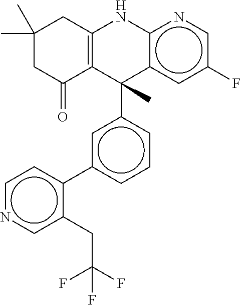 |
| 654RR | 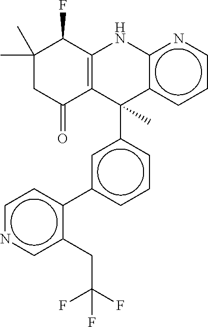 |
| 654SR | 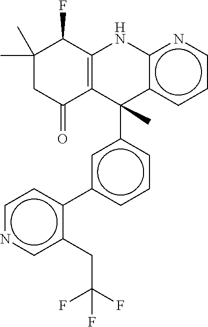 |
| 654RS | 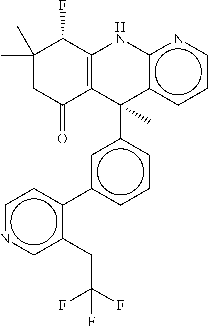 |
| 654SS | 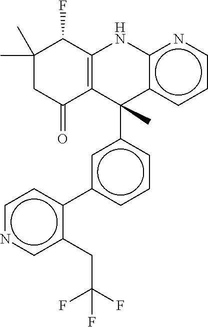 |
| 655RS | 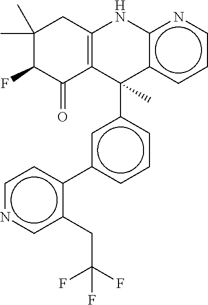 |
| 655SS | 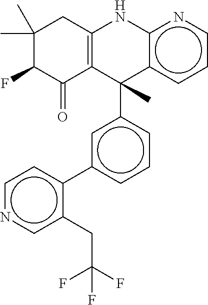 |
| 655RR | 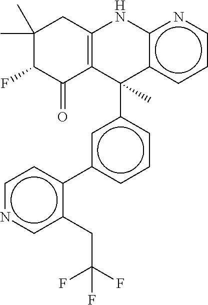 |
| 655SR | 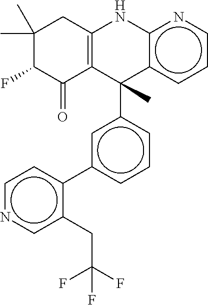 |
| 656R | 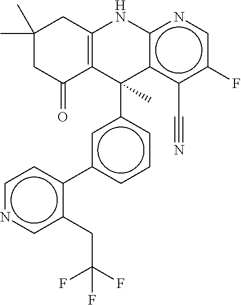 |
| 656S | 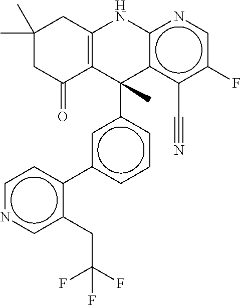 |
| 657R | 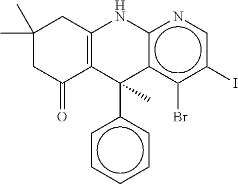 |
| 657S | 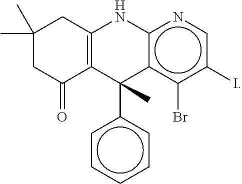 |

18. The compound of claim 1, or a pharmaceutically acceptable salt, solvate, hydrate, polymorph, co-crystal, tautomer, stereoisomer, isotopically labeled compound, or prodrug thereof, wherein the compound is of any one of the formulae:

| No. | Formula |
|---|---|
| 1002R | 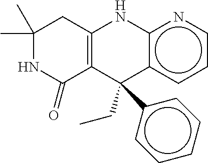 |
| 1002S | 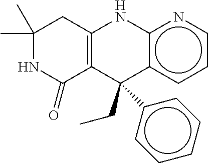 |
| 1004R | 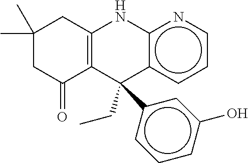 |
| 1004S | 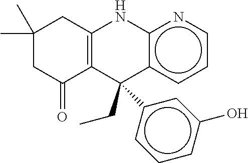 |
| 1006RR | 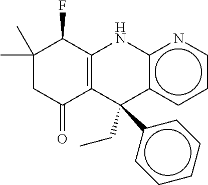 |
| 1006SR | 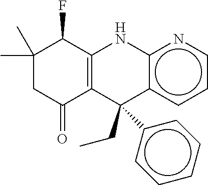 |
| 1006RS | 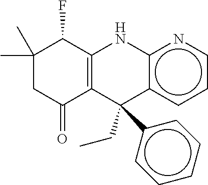 |
| 1006SS | 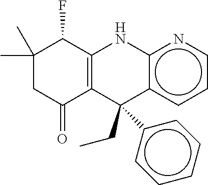 |
| 1007R | 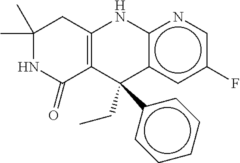 |
| 1007S | 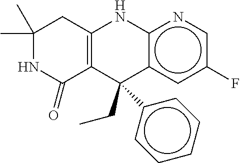 |
| 1008R | 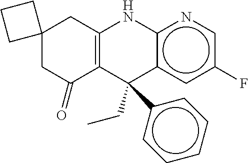 |
| 1008S | 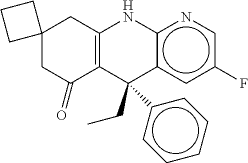 |
| 1009RR | 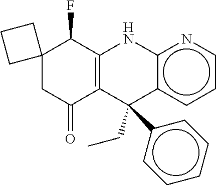 |
| 1009SR | 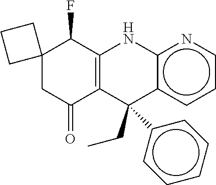 |
| 1009RS | 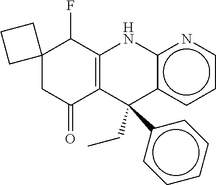 |
| 1009SS | 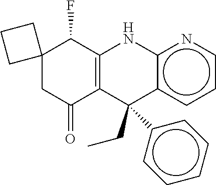 |
| 1010R | 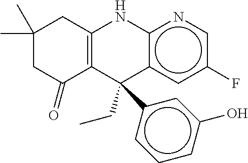 |
| 1010S | 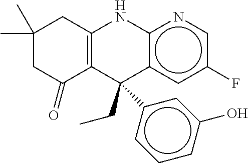 |
| 1011R | 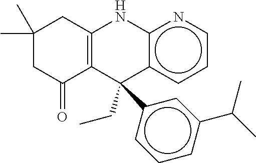 |
| 1011S | 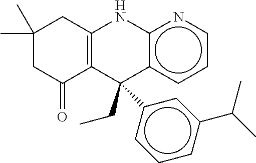 |
| 1012R | 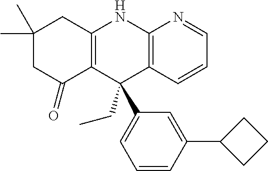 [SMILES: CC[C@]1(c2cccc(C3CCC3)c2)C2=C(CC(C)(C)CC2=O)Nc2ncccc21] |
| 1012S | 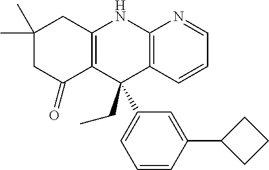 [SMILES: CC[C@@]1(c2cccc(C3CCC3)c2)C2=C(CC(C)(C)CC2=O)Nc2ncccc21] |
| 1014R | 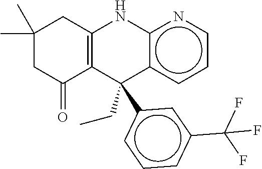 |
| 1014S | 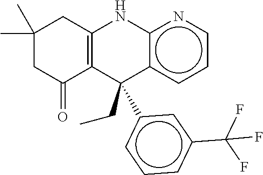 |
| 1015R | 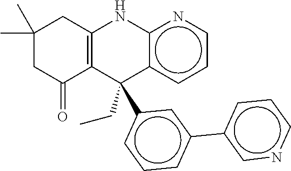 |
| 1015S | 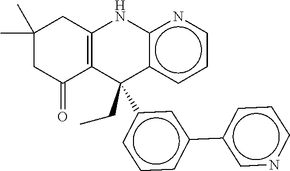 |
| 1016R | 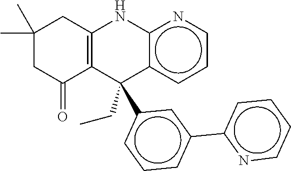 |
| 1016S | 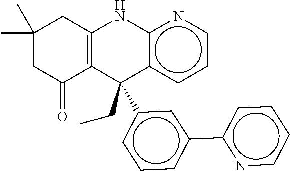 |
| 1017R | 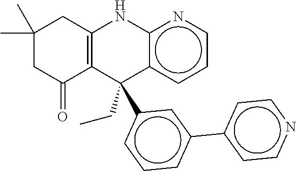 |
| 1017S | 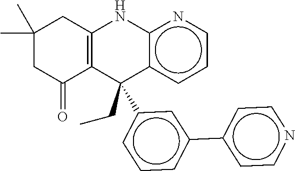 |
| 1018R | 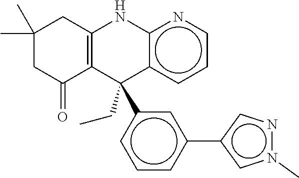 |
| 1018S | 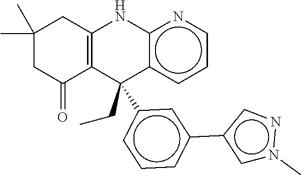 |
| 1019R | 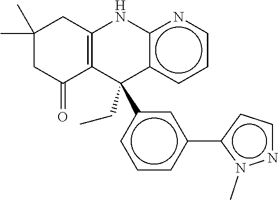 |
| 1019S | 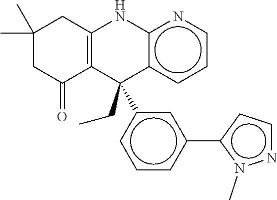 |
| 1020R | 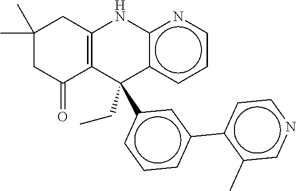 |
| 1020S | 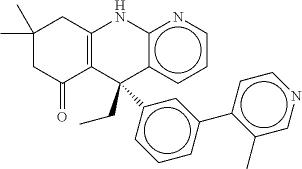 |
| 1021R | 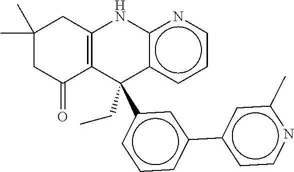 |
| 1021S | 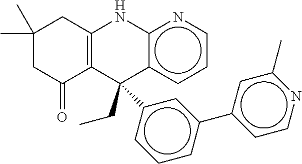 |
| 1022R | 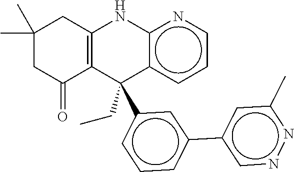 |
| 1022S | 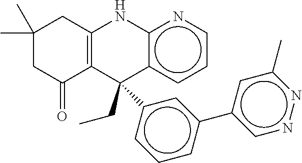 |
| 1023R | 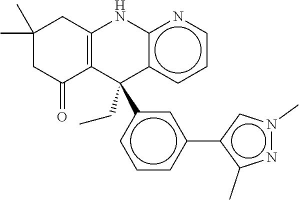 |
| 1023S | 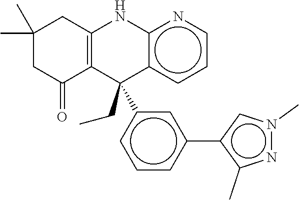 |
| 1024R | 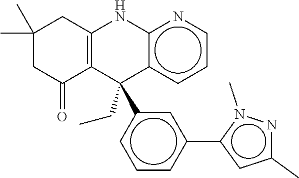 |
| 1024S | 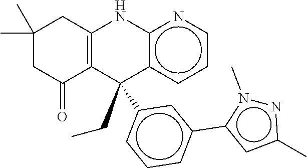 |
| 1025R | 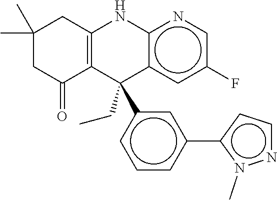 |
| 1025S | 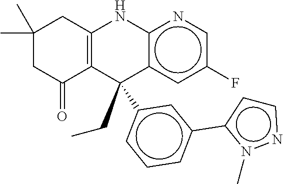 |
| 1027R | 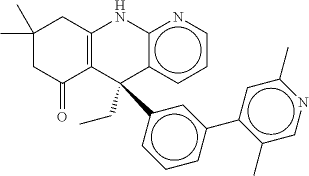 |
| 1027S | 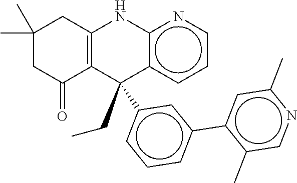 |
| 1028R | 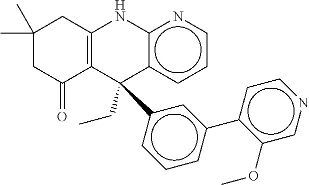 |
| 1028S | 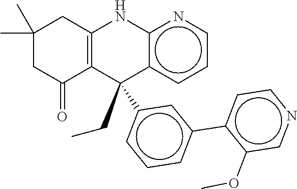 |
| 1031R | 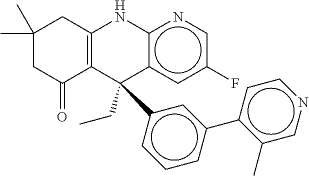 |
| 1031S | 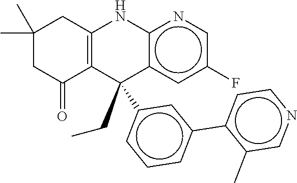 |
| 1032R | 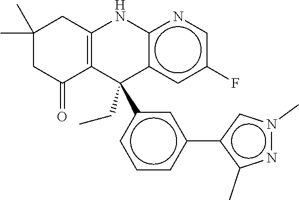 |
| 1032S | 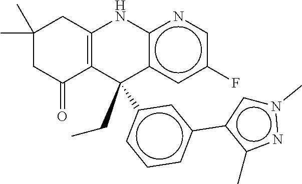 |
| 1033R | 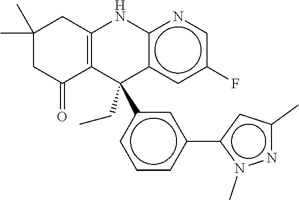 |
| 1033S | 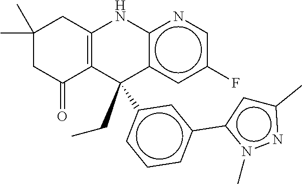 |
| 1034R | 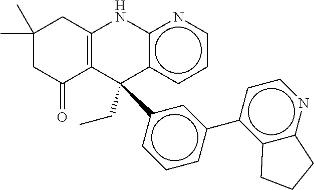 |
| 1034S | 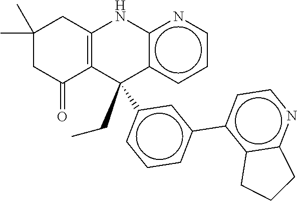 |
| 1035R | 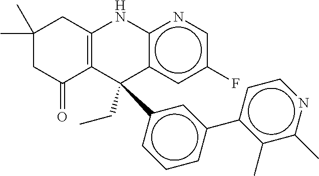 |
| 1035S | 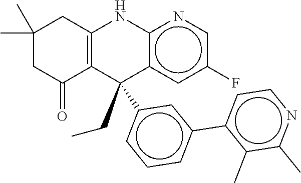 |
| 1037R | 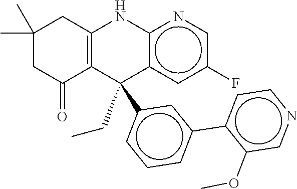 |
| 1037S | 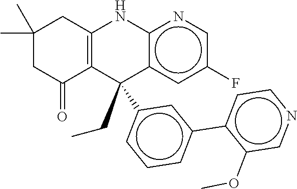 |
| 1038R | 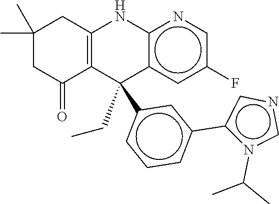 |
| 1038S | 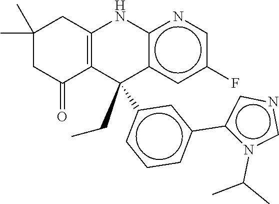 |
| 1039R | 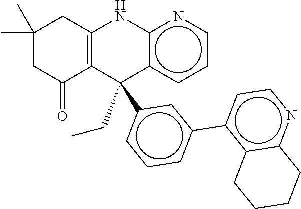 |
| 1039S | 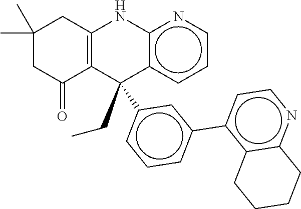 |

19. A pharmaceutical composition comprising:
the compound of any one of claims 1-18, or a pharmaceutically acceptable salt, solvate, hydrate, polymorph, co-crystal, tautomer, stereoisomer, isotopically labeled compound, or prodrug thereof; and
optionally a pharmaceutically acceptable excipient.

20. A method of treating or preventing a disease in a subject in need thereof, the method comprising administering to the subject an effective amount of the compound of any one of claims 1-18, or a pharmaceutically acceptable salt, solvate, hydrate, polymorph, co-crystal, tautomer, stereoisomer, isotopically labeled compound, or prodrug thereof, or the pharmaceutical composition of claim 19.

21. A method of inhibiting the aberrantly high activity and/or production of a glycogen synthase kinase 3 (GSK3) in a subject in need thereof, cell, tissue, or biological sample, the method comprising administering to the subject or contacting the cell, tissue, or biological sample with an effective amount of the compound of any one of claims 1-18, or a pharmaceutically acceptable salt, solvate, hydrate, polymorph, co-crystal, tautomer, stereoisomer, isotopically labeled compound, or prodrug thereof, or the pharmaceutical composition of claim 19.
